(19) United States
(12) Patent Application Publication
(10) Pub. No.: US 2026/0365628 A1
(43) Pub. Date: Nov. 19, 2026

(54) SUBSTITUTED 3,4-DIHYDROQUINOLINONE INHIBITORS OF TSHR

(71) Applicant: Septerna, Inc., South San Francisco, CA (US)

(72) Inventors: Steven J. McKerrall, San Francisco, CA (US); Daniel D. Long, San Francisco, CA (US); Xiaohui Du, Belmont, CA (US); Deniz Aydin, Redwood City, CA (US); Matthew Volgraf, Oakland, CA (US)

(21) Appl. No.: 19/099,412

(22) PCT Filed: Jul. 28, 2023
(86) PCT No.: PCT/US2023/028950

§ 371 Date: Jan. 28, 2025

Related U.S. Application Data
(60) Provisional application No. 63/393,411, filed on Jul. 29, 2022.

(51) Int. Cl. C07D 241/44; A61K 31/421; A61K 31/4704; A61K 31/4985; A61K 31/517; A61K 31/538; C07D 209/34; C07D 215/227; C07D 239/80; C07D 263/58; C07D 265/36; C07D 471/04

(52) U.S. Cl. CPC C07D 241/44; A61K 31/421; A61K 31/4704; A61K 31/4985; A61K 31/517; A61K 31/538; C07D 209/34; C07D 215/227; C07D 239/80; C07D 263/58; C07D 265/36; C07D 471/04

(57) ABSTRACT

Disclosed are compounds that are thyroid stimulating hormone receptor antagonists, and methods useful for preventing or treating a thyroid disease.

### CROSS-REFERENCE TO RELATED APPLICATIONS

This application claims the benefit of priority to U.S. Provisional Application No. 63/393,411, filed Jul. 29, 2022; the contents of which are hereby incorporated by reference in their entirety.

### BACKGROUND

About 40% of hyperthyroidism patients suffer from Graves' disease (Morbus Basedow), an autoimmune disease in which autoantibodies activate the thyrotropin receptor, mimicking its natural hormone ligand, the thyroid-stimulating hormone (TSH). This pathological activation of TSH-Receptor (TSHR) leads to uncontrolled production of thyroid hormones (e.g., T3 and T4) causing hyperthyroidism. TSH and the TSHR are important proteins for controlling thyroid function. TSHR is primarily expressed in follicular epithelial cells of the thyroid gland, but also is expressed in a variety of additional cell types, such as retro-orbital fibroblasts, kidney cells, adipocytes and bone cells. TSH binds to its receptor and leads to the stimulation of second messenger pathways involving predominantly cAMP. Inositol 1,4,5-triphosphate (IP3) and diacylglycerol (DAG) pathways are also activated at higher TSH concentrations.

The treatment of choice applied in the clinics for decades involves thyrostatic drugs that block the production of thyroid hormones. These medications play a role further downstream in the signal cascade of the thyroid upon activation of the TSHR. Since thyroid hormones T3 and T4 are secreted in the thyroid gland, thyrostatic drugs induce an inhibition of their synthesis. Thus, the current primary anti-thyroid treatment does not target the causative molecular activation of the TSHR by autoantibodies and patients are therefore burdened by a rate of at least 5% adverse effects. This demands frequent controls of the thyroid hormone levels and adjustments of thyrostatics dosage. In contrast to these drugs, which regulate the thyroid hormone level, another promising target is the TSHR itself. However, small allosteric antagonists acting directly at the TSHR are not available on the market yet.

Additionally, about 25% of Graves' disease patients also develop an orbitopathy referred to as “Graves' ophthalmopathy”, a related organ-specific autoimmune disease affecting the appearance and functioning of the eyes. There is considerable evidence that expression of the TSHR in the orbital fibroblasts and orbital adipocytes behind the eye may contribute to this difficult-to-treat orbitopathy, and thyroid stimulating antibodies titer tend to correlate with severity of Grave's ophthalmopathy. Orbital fibroblast has been recognized as primary target cells of autoimmune attack and TSHR acts as a primary autoantigen in Grave's ophthalmopathy. The pathological activation of TSHR leads to the production of the extracellular matrix by involvement of hyaluronanic acid, fibrosis and swelling of extraocular muscle as well as adipogenesis of orbital fibroblasts (orbital fat expansion). The increase in tissue volume in the orbit often causes diplopia and compression of the optic nerve and exophthalmos. Thus, the TSHR is also potential target for pharmacological intervention of Grave's ophthalmology and thyroid eye disease.

Accordingly, there is a need in the art to provide additional means for the treatment of hyperthyroidism, in particular compounds that act as TSHR antagonists.

### SUMMARY

One aspect of the invention provides compounds, compositions, and methods useful for preventing or treating a thyroid disease, such as hyperthyroidism, Grave's disease, Grave's opthalmopathy, or thyroid eye disease.

Accordingly, provided herein in some embodiments is a compound having the structure of Formula (I):

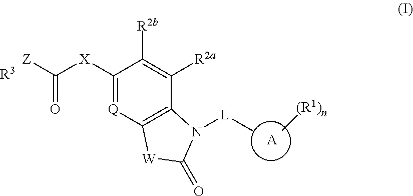

or a pharmaceutically acceptable salt thereof,
wherein:
   A is (C₆-C₁₀)aryl, 5- to 10-membered heteroaryl, (C₃-C₈)cycloalkyl, or 4- to 7-membered heterocycloalkyl;
   n is 0, 1, 2, 3, 4, or 5;
   R¹ is independently for each occurrence (C₁-C₆)alkyl, (C₂-C₆)alkynyl, (C₂-C₆)alkenyl, (C₃-C₈)cycloalkyl, (C₃-C₈)cycloalkoxy, (C₆-C₁₀)aryl, (C₆-C₁₀)aryloxy, (C₆-C₁₀)aryl(C₁-C₆)alkyl, 4- to 7-membered heterocycloalkyl, (C₁-C₆)alkoxy, (C₁-C₆)alkoxy(C₁-C₆)alkyl, halo, cyano, amino, formyl, carboxy, or alkoxycarbonyl,
   wherein (C₁-C₆)alkyl, (C₂-C₆)alkynyl, (C₂-C₆)alkenyl, (C₃-C₈)cycloalkyl, C₃-C₈)cycloalkoxy, (C₆-C₁₀)aryl, (C₆-C₁₀)aryloxy, (C₆-C₁₀)aryl(C₁-C₆)alkyl, (C₁-C₆)alkoxy, (C₁-C₆)alkoxy(C₁-C₆)alkyl, and 4- to 7-membered heterocycloalkyl are each optionally substituted with one or more substituents independently selected from halo, hydroxy, cyano, amido, sulfonamido, (C₁-C₆)alkyl, (C₃-C₈)cycloalkyl, 4- to 7-membered heterocycloalkyl, 5- to 6-membered heteroaryl, (C₁-C₆)alkoxy, (C₁-C₆)haloalkoxy, (C₁-C₆)fluoroalkoxy, 5- to 6-membered heteroaryloxy, or
   two vicinal occurrences of R¹ taken together with the atoms to which they are attached form a fused 5- to 7-membered cycloalkyl, 5- to 7-membered heterocycloalkyl ring, 5- to 6-membered heteroaryl ring or phenyl ring, any of which is optionally substituted with one or more substituents independently selected from halo, hydroxy, cyano, amido, sulfonamido, (C₁-C₆)alkyl, (C₁-C₆)haloalkyl (C₃-C₈)cycloalkyl, and (C₁-C₆)alkoxy;
   Q is N or CR^{2c};
   R^{2a}, R^{2b}, and R^{2c}, are each independently hydrogen, halo, cyano, (C₁-C₆)alkyl, or (C₃-C₈)cycloalkyl, wherein (C₁-C₆)alkyl is optionally substituted with one or more substituents independently selected from halo, hydroxy, and (C₁-C₆)alkoxy;
   R³ is (C₁-C₆)alkyl, (C₃-C₈)cycloalkyl, (C₃-C₈)cycloalkyl(C₁-C₆)alkyl), (C₆-C₁₀)aryl, (C₆-C₁₀)aryl(C₁-C₆)alkyl), or 4- to 7-membered heterocycloalkyl; each of which is optionally substituted with one or more substituents independently selected from halo, cyano, (C₁-C₆)haloalkyl, hydroxy, (C₁-C₆)alkoxy(C₁-C₆)alkyl, and (C₁-C₆)alkoxy;
   X and Z are independently NR⁴, C(R⁵)₂, or O;
   R⁴ and R⁵ independently for each occurrence hydrogen or (C₁-C₆)alkyl, wherein (C₁-C₆)alkyl is optionally substituted with one or more substituents independently selected from halo, hydroxy, and (C₁-C₆)alkoxy, or when Z is NR⁴, R³ and R⁴ taken together with the nitrogen to which they are attached form a 4- to 7-member heterocycloalkyl optionally substituted with one or more (C₁-C₆)alkyl or halo;
   L is (C₁-C₆)alkylene optionally substituted with one or more substituents independently selected from halo, hydroxy, C₁-C₆)alkoxy, and cyano;
   W represents:
      —C(R⁶)₂—C(R⁷)₂—,
      —C(R⁷)₂—,
      —O—C(R⁸)₂—,
      C(R⁸)₂—O—,
      C(R⁸)₂—NR⁹—,
      —N═C(R¹⁰)—, or
      —O—; and
   R⁶, R⁷, R⁸, R⁹ and R¹⁰ are independently for each occurrence hydrogen, (C₁-C₆)alkyl, or NH₂, wherein (C₁-C₆)alkyl is optionally substituted with one or more substituents independently selected from halo, cyano, hydroxy, (C₁-C₆)alkoxy(C₁-C₆)alkyl, and (C₁-C₆)alkoxy.

In some embodiments a compound has the structure of Formula (II):

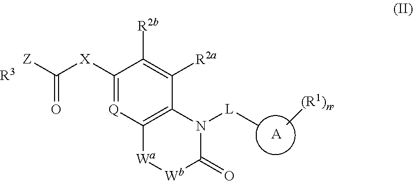

or a pharmaceutically acceptable salt thereof,
wherein
   A is (C₆-C₁₀)aryl, 5- to 10-membered heteroaryl, (C₃-C₈)cycloalkyl, or 4- to 7-membered heterocycloalkyl;
   n is 0, 1, 2, 3, 4, or 5;
   R¹ is independently for each occurrence (C₁-C₆)alkyl, (C₂-C₆)alkynyl, (C₂-C₆)alkenyl, (C₃-C₈)cycloalkyl, (C₃-C₈)cycloalkoxy, (C₆-C₁₀)aryl, (C₆-C₁₀)aryloxy, (C₆-C₁₀)aryl(C₁-C₆)alkyl, 4- to 7-membered heterocycloalkyl, (C₁-C₆)alkoxy, (C₁-C₆)alkoxy(C₁-C₆)alkyl, halo, cyano, amino, formyl, carboxy, or alkoxycarbonyl,
   wherein (C₁-C₆)alkyl, (C₂-C₆)alkynyl, (C₂-C₆)alkenyl, (C₃-C₈)cycloalkyl, C₃-C₈)cycloalkoxy, (C₆-C₁₀)aryl, (C₆-C₁₀)aryloxy, (C₆-C₁₀)aryl(C₁-C₆)alkyl, (C₁-C₆)alkoxy, (C₁-C₆)alkoxy(C₁-C₆)alkyl, and 4- to 7-membered heterocycloalkyl are each optionally substituted with one or more substituents independently selected from halo, hydroxy, cyano, amido, sulfonamido, (C₁-C₆)alkyl, (C₃-C₈)cycloalkyl, 4- to 7-membered heterocycloalkyl, 5- to 6-membered heteroaryl, (C₁-C₆)alkoxy, (C₁-C₆)haloalkoxy, (C₁-C₆)fluoroalkoxy, 5- to 6-membered heteroaryloxy, or
   two vicinal occurrences of R¹ taken together with the atoms to which they are attached form a fused 5- to 7-membered cycloalkyl, 5- to 7-membered heterocycloalkyl ring, 5- to 6-membered heteroaryl ring or phenyl ring, any of which is optionally substituted with one or more substituents independently selected from halo, hydroxy, cyano, amido, sulfonamido, (C₁-C₆)alkyl, (C₁-C₆)haloalkyl (C₃-C₈)cycloalkyl, and (C₁-C₆)alkoxy;
   Q is N or CR^{2c};
   R^{2a}, R^{2b}, and R^{2c}, are each independently hydrogen, halo, cyano, (C₁-C₆)alkyl, or (C₃-C₈)cycloalkyl, wherein (C₁-C₆)alkyl is optionally substituted with one or more substituents independently selected from halo, hydroxy, and (C₁-C₆)alkoxy;
   R³ is (C₁-C₆)alkyl, (C₃-C₈)cycloalkyl, (C₃-C₈)cycloalkyl(C₁-C₆)alkyl), (C₆-C₁₀)aryl, (C₆-C₁₀)aryl(C₁-C₆)alkyl), or 4- to 7-membered heterocycloalkyl; each of which is optionally substituted with one or more substituents independently selected from halo, cyano, (C₁-C₆)haloalkyl, hydroxy, (C₁-C₆)alkoxy(C₁-C₆)alkyl, and (C₁-C₆)alkoxy;
   X and Z are independently NR⁴, C(R⁵)₂, or O;
   R⁴ and R⁵ independently for each occurrence hydrogen or (C₁-C₆)alkyl, wherein (C₁-C₆)alkyl is optionally substituted with one or more substituents independently selected from halo, hydroxy, and (C₁-C₆)alkoxy, or when Z is NR⁴, R³ and R⁴ taken together with the nitrogen to which they are attached form a 4- to 7-member heterocycloalkyl optionally substituted with one or more (C₁-C₆)alkyl or halo;
   L is (C₁-C₆)alkylene optionally substituted with one or more substituents independently selected from halo, hydroxy, C₁-C₆)alkoxy, and cyano;
   W represents:
      —C(R⁶)₂—C(R⁷)₂—,
      —C(R⁷)₂—,
      —O—C(R⁸)₂—,
      C(R⁸)₂—O—,
      C(R⁸)₂—NR⁹—,
      —N═C(R¹⁰)—, or
      —O—; and
   R⁶, R⁷, R⁸, R⁹ and R¹⁰ are independently for each occurrence hydrogen, (C₁-C₆)alkyl, or NH₂, wherein (C₁-C₆)alkyl is optionally substituted with one or more substituents independently selected from halo, cyano, hydroxy, (C₁-C₆)alkoxy(C₁-C₆)alkyl, and (C₁-C₆)alkoxy.

In some embodiments a compound has the structure of Formula (III):

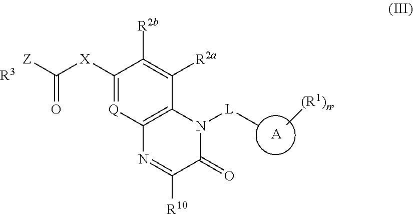

or a pharmaceutically acceptable salt thereof,
wherein:
   A is (C₆-C₁₀)aryl, 5- to 10-membered heteroaryl, (C₃-C₈)cycloalkyl, or 4- to 7-membered heterocycloalkyl;
   n is 0, 1, 2, 3, 4, or 5;
   R¹ is independently for each occurrence (C₁-C₆)alkyl, (C₂-C₆)alkynyl, (C₂-C₆)alkenyl, (C₃-C₈)cycloalkyl, (C₃-C₈)cycloalkoxy, (C₆-C₁₀)aryl, (C₆-C₁₀)aryloxy, (C₆-C₁₀)aryl(C₁-C₆)alkyl, 4- to 7-membered heterocycloalkyl, (C₁-C₆)alkoxy, (C₁-C₆)alkoxy(C₁-C₆)alkyl, halo, cyano, amino, formyl, carboxy, or alkoxycarbonyl,
   wherein (C₁-C₆)alkyl, (C₂-C₆)alkynyl, (C₂-C₆)alkenyl, (C₃-C₈)cycloalkyl, C₃-C₈)cycloalkoxy, (C₆-C₁₀)aryl, (C₆-C₁₀)aryloxy, (C₆-C₁₀)aryl(C₁-C₆)alkyl, (C₁-C₆)alkoxy, (C₁-C₆)alkoxy(C₁-C₆)alkyl, and 4- to 7-membered heterocycloalkyl are each optionally substituted with one or more substituents independently selected from halo, hydroxy, cyano, amido, sulfonamido, (C₁-C₆)alkyl, (C₃-C₈)cycloalkyl, 4- to 7-membered heterocycloalkyl, 5- to 6-membered heteroaryl, (C₁-C₆)alkoxy, (C₁-C₆)haloalkoxy, (C₁-C₆)fluoroalkoxy, 5- to 6-membered heteroaryloxy, or
   two vicinal occurrences of R¹ taken together with the atoms to which they are attached form a fused 5- to 7-membered cycloalkyl, 5- to 7-membered heterocycloalkyl ring, 5- to 6-membered heteroaryl ring or phenyl ring, any of which is optionally substituted with one or more substituents independently selected from halo, hydroxy, cyano, amido, sulfonamido, (C₁-C₆)alkyl, (C₁-C₆)haloalkyl (C₃-C₈)cycloalkyl, and (C₁-C₆)alkoxy;
   Q is N or CR^{2c};
   R^{2a}, R^{2b}, and R^{2c}, are each independently hydrogen, halo, cyano, (C₁-C₆)alkyl, or (C₃-C₈)cycloalkyl, wherein (C₁-C₆)alkyl is optionally substituted with one or more substituents independently selected from halo, hydroxy, and (C₁-C₆)alkoxy;
   R³ is (C₁-C₆)alkyl, (C₃-C₈)cycloalkyl, (C₃-C₈)cycloalkyl(C₁-C₆)alkyl), (C₆-C₁₀)aryl, (C₆-C₁₀)aryl(C₁-C₆)alkyl), or 4- to 7-membered heterocycloalkyl; each of which is optionally substituted with one or more substituents independently selected from halo, cyano, (C₁-C₆)alkyl, (C₁-C₆)haloalkyl, hydroxy, (C₁-C₆)alkoxy(C₁-C₆)alkyl, and (C₁-C₆)alkoxy;
   X and Z are independently NR⁴, C(R⁵)₂, or O;
R⁴ and R⁵ independently for each occurrence hydrogen or (C₁-C₆)alkyl, wherein (C₁-C₆)alkyl is optionally substituted with one or more substituents independently selected from halo, hydroxy, and (C₁-C₆)alkoxy, or when Z is NR⁴, R³ and R⁴ taken together with the nitrogen to which they are attached form a 4- to 7-member heterocycloalkyl optionally substituted with one or more (C₁-C₆)alkyl or halo, or when Z is C(R⁵)₂, R³ and one R⁵ taken together with the carbon to which they are attached form a 4, (C₃-C₈)cycloalkyl optionally substituted with one or more (C₁-C₆)alkyl or halo;
   L is (C₁-C₆)alkylene optionally substituted with one or more substituents independently selected from halo, hydroxy, C₁-C₆)alkoxy, and cyano;
R¹⁰ is hydrogen, (C₁-C₆)alkyl, or NH₂, wherein (C₁-C₆)alkyl is optionally substituted with one or more substituents independently selected from halo, cyano, hydroxy, (C₁-C₆)alkoxy(C₁-C₆)alkyl, and (C₁-C₆)alkoxy.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to an cycloalkyl group as hereinbefore defined substituted with at least one halogen.

“Cycloheteroalkyl” refers to an cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” or “heterocycloalkyl” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, chromane, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

### Compounds of the Invention

One aspect of the invention relates to a compound of Formula (I):

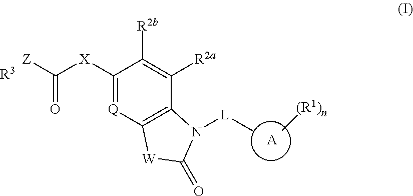

or a pharmaceutically acceptable salt thereof,
wherein:
   A is (C₆-C₁₀)aryl, 5- to 10-membered heteroaryl, (C₃-C₈)cycloalkyl, or 4- to 7-membered heterocycloalkyl;
   n is 0, 1, 2, 3, 4, or 5;
   R¹ is independently for each occurrence (C₁-C₆)alkyl, (C₂-C₆)alkynyl, (C₂-C₆)alkenyl, (C₃-C₈)cycloalkyl, (C₃-C₈)cycloalkoxy, (C₆-C₁₀)aryl, (C₆-C₁₀)aryloxy, (C₆-C₁₀)aryl(C₁-C₆)alkyl, 4- to 7-membered heterocycloalkyl, (C₁-C₆)alkoxy, (C₁-C₆)alkoxy(C₁-C₆)alkyl, halo, cyano, amino, formyl, carboxy, or alkoxycarbonyl,
   wherein (C₁-C₆)alkyl, (C₂-C₆)alkynyl, (C₂-C₆)alkenyl, (C₃-C₈)cycloalkyl, C₃-C₈)cycloalkoxy, (C₆-C₁₀)aryl, (C₆-C₁₀)aryloxy, (C₆-C₁₀)aryl(C₁-C₆)alkyl, (C₁-C₆)alkoxy, (C₁-C₆)alkoxy(C₁-C₆)alkyl, and 4- to 7-membered heterocycloalkyl are each optionally substituted with one or more substituents independently selected from halo, hydroxy, cyano, amido, sulfonamido, (C₁-C₆)alkyl, (C₃-C₈)cycloalkyl, 4- to 7-membered heterocycloalkyl, 5- to 6-membered heteroaryl, (C₁-C₆)alkoxy, (C₁-C₆)haloalkoxy, (C₁-C₆)fluoroalkoxy, 5- to 6-membered heteroaryloxy, or
   two vicinal occurrences of R¹ taken together with the atoms to which they are attached form a fused 5- to 7-membered cycloalkyl, 5- to 7-membered heterocycloalkyl ring, 5- to 6-membered heteroaryl ring or phenyl ring, any of which is optionally substituted with one or more substituents independently selected from halo, hydroxy, cyano, amido, sulfonamido, (C₁-C₆)alkyl, (C₁-C₆)haloalkyl (C₃-C₈)cycloalkyl, and (C₁-C₆)alkoxy;
Q is N or CR^{2c};
R^{2a}, R^{2b}, and R^{2c}, are each independently hydrogen, halo, cyano, (C₁-C₆)alkyl, or (C₃-C₈)cycloalkyl, wherein (C₁-C₆)alkyl is optionally substituted with one or more substituents independently selected from halo, hydroxy, and (C₁-C₆)alkoxy;
   R³ is (C₁-C₆)alkyl, (C₃-C₈)cycloalkyl, (C₃-C₈)cycloalkyl(C₁-C₆)alkyl), (C₆-C₁₀)aryl, (C₆-C₁₀)aryl(C₁-C₆)alkyl), or 4- to 7-membered heterocycloalkyl; each of which is optionally substituted with one or more substituents independently selected from halo, cyano, (C₁-C₆)haloalkyl, hydroxy, (C₁-C₆)alkoxy(C₁-C₆)alkyl, and (C₁-C₆)alkoxy;
   X and Z are independently NR⁴, C(R⁵)₂, or O;
   R⁴ and R⁵ independently for each occurrence hydrogen or (C₁-C₆)alkyl, wherein (C₁-C₆)alkyl is optionally substituted with one or more substituents independently selected from halo, hydroxy, and (C₁-C₆)alkoxy, or when Z is NR⁴, R³ and R⁴ taken together with the nitrogen to which they are attached form a 4- to 7-member heterocycloalkyl optionally substituted with one or more (C₁-C₆)alkyl or halo;
   L is (C₁-C₆)alkylene optionally substituted with one or more substituents independently selected from halo, hydroxy, C₁-C₆)alkoxy, and cyano;
   W represents:
      —C(R⁶)₂—C(R⁷)₂—,
      —C(R⁷)₂—,
      —O—C(R⁸)₂—,
      —C(R⁸)₂—O—,
      —C(R⁸)₂—NR⁹—,
      —N═C(R¹⁰)—, or
      —O—; and
   R⁶, R⁷, R⁸, R⁹ and R¹⁰ are independently for each occurrence hydrogen, (C₁-C₆)alkyl, or NH₂, wherein (C₁-C₆)alkyl is optionally substituted with one or more substituents independently selected from halo, cyano, hydroxy, (C₁-C₆)alkoxy(C₁-C₆)alkyl, and (C₁-C₆)alkoxy.

In some embodiments of the compound of Formula (I),
A is (C₆-C₁₀)aryl or 5- to 10-membered heteroaryl;
n is 0, 1, 2, 3, 4, or 5;
R¹ is independently for each occurrence (C₁-C₆)alkyl, (C₃-C₈)cycloalkyl, 4- to 7-membered heterocycloalkyl, (C₁-C₆)alkoxy, halo, cyano, formyl, carboxy, or alkoxycarbonyl, wherein (C₁-C₆)alkyl, (C₃-C₈)cycloalkyl, (C₁-C₆)alkoxy, and 4- to 7-membered heterocycloalkyl are each optionally substituted with one or more substituents independently selected from halo, hydroxy, cyano, amido, sulfonamido, (C₁-C₆)alkyl, (C₃-C₈)cycloalkyl, and (C₁-C₆)alkoxy, or
two vicinal occurrences of R¹ taken together with the atoms to which they are attached form a fused 5- to 7-membered cycloalkyl or 5- to 7-membered heterocycloalkyl ring, either of which is optionally substituted with one or more substituents independently selected from halo, hydroxy, cyano, amido, sulfonamido, (C₁-C₆)alkyl, (C₃-C₈)cycloalkyl, and (C₁-C₆)alkoxy;
Q is N or CR^{2c};
R^{2a}, R^{2b}, and R^{2c}, are each independently hydrogen, halo, cyano, or (C₁-C₆)alkyl, wherein (C₁-C₆)alkyl is optionally substituted with one or more substituents independently selected from halo, hydroxy, and (C₁-C₆)alkoxy;
R³ is (C₁-C₆)alkyl optionally substituted with one or more substituents independently selected from halo, hydroxy, and (C₁-C₆)alkoxy;
X and Z are independently NR⁴, C(R⁵)₂, or O;
R⁴ and R⁵ independently for each occurrence hydrogen or (C₁-C₆)alkyl, wherein (C₁-C₆)alkyl is optionally substituted with one or more substituents independently selected from halo, hydroxy, and (C₁-C₆)alkoxy;
L is (C₁-C₆)alkylene optionally substituted with one or more substituents independently selected from halo, hydroxy, C₁-C₆)alkoxy, and cyano;
W represents:
   —C(R⁶)₂—C(R⁷)₂—,
   —C(R⁷)₂—,
   —O—C(R⁸)₂—,
   —C(R⁸)₂—O—,
   —C(R⁸)₂—NR⁹—,
   —N═C(R¹⁰)—, or
   —O—; and
R⁶, R⁷, R⁸, R⁹ and R¹⁰ are independently for each occurrence hydrogen or (C₁-C₆)alkyl, wherein (C₁-C₆)alkyl is optionally substituted with one or more substituents independently selected from halo, hydroxy, and (C₁-C₆)alkoxy.:

Another aspect of the invention relates to a compound Formula (II):

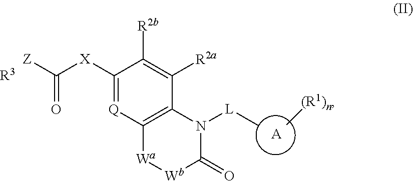

or a pharmaceutically acceptable salt thereof,
wherein
   A is (C₆-C₁₀)aryl, 5- to 10-membered heteroaryl, (C₃-C₈)cycloalkyl, or 4- to 7-membered heterocycloalkyl;
   n is 0, 1, 2, 3, 4, or 5;
   R¹ is independently for each occurrence (C₁-C₆)alkyl, (C₂-C₆)alkynyl, (C₂-C₆)alkenyl, (C₃-C₈)cycloalkyl, (C₃-C₈)cycloalkoxy, (C₆-C₁₀)aryl, (C₆-C₁₀)aryloxy, (C₆-C₁₀)aryl(C₁-C₆)alkyl, 4- to 7-membered heterocycloalkyl, (C₁-C₆)alkoxy, (C₁-C₆)alkoxy(C₁-C₆)alkyl, halo, cyano, amino, formyl, carboxy, or alkoxycarbonyl,
   wherein (C₁-C₆)alkyl, (C₂-C₆)alkynyl, (C₂-C₆)alkenyl, (C₃-C₈)cycloalkyl, C₃-C₈)cycloalkoxy, (C₆-C₁₀)aryl, (C₆-C₁₀)aryloxy, (C₆-C₁₀)aryl(C₁-C₆)alkyl, (C₁-C₆)alkoxy, (C₁-C₆)alkoxy(C₁-C₆)alkyl, and 4- to 7-membered heterocycloalkyl are each optionally substituted with one or more substituents independently selected from halo, hydroxy, cyano, amido, sulfonamido, (C₁-C₆)alkyl, (C₃-C₈)cycloalkyl, 4- to 7-membered heterocycloalkyl, 5- to 6-membered heteroaryl, (C₁-C₆)alkoxy, (C₁-C₆)haloalkoxy, (C₁-C₆)fluoroalkoxy, 5- to 6-membered heteroaryloxy, or
   two vicinal occurrences of R¹ taken together with the atoms to which they are attached form a fused 5- to 7-membered cycloalkyl, 5- to 7-membered heterocycloalkyl ring, 5- to 6-membered heteroaryl ring or phenyl ring, any of which is optionally substituted with one or more substituents independently selected from halo, hydroxy, cyano, amido, sulfonamido, (C₁-C₆)alkyl, (C₁-C₆)haloalkyl (C₃-C₈)cycloalkyl, and (C₁-C₆)alkoxy;
   Q is N or CR^{2c};
   R^{2a}, R^{2b}, and R^{2c}, are each independently hydrogen, halo, cyano, (C₁-C₆)alkyl, or (C₃-C₈)cycloalkyl, wherein (C₁-C₆)alkyl is optionally substituted with one or more substituents independently selected from halo, hydroxy, and (C₁-C₆)alkoxy;
   R³ is (C₁-C₆)alkyl, (C₃-C₈)cycloalkyl, (C₃-C₈)cycloalkyl(C₁-C₆)alkyl), (C₆-C₁₀)aryl, (C₆-C₁₀)aryl(C₁-C₆)alkyl), or 4- to 7-membered heterocycloalkyl; each of which is optionally substituted with one or more substituents independently selected from halo, cyano, (C₁-C₆)haloalkyl, hydroxy, (C₁-C₆)alkoxy(C₁-C₆)alkyl, and (C₁-C₆)alkoxy;
   X and Z are independently NR⁴, C(R⁵)₂, or O;
   R⁴ and R⁵ independently for each occurrence hydrogen or (C₁-C₆)alkyl, wherein (C₁-C₆)alkyl is optionally substituted with one or more substituents independently selected from halo, hydroxy, and (C₁-C₆)alkoxy, or when Z is NR⁴, R³ and R⁴ taken together with the nitrogen to which they are attached form a 4- to 7-member heterocycloalkyl optionally substituted with one or more (C₁-C₆)alkyl or halo;
   L is (C₁-C₆)alkylene optionally substituted with one or more substituents independently selected from halo, hydroxy, C₁-C₆)alkoxy, and cyano;
   W represents:
      —C(R⁶)₂—C(R⁷)₂—,
      —C(R⁷)₂—,
      —O—C(R⁸)₂—,
      C(R⁸)₂—O—,
      C(R⁸)₂—NR⁹—,
      —N═C(R¹⁰)—, or
      —O—; and
   R⁶, R⁷, R⁸, R⁹ and R¹⁰ are independently for each occurrence hydrogen, (C₁-C₆)alkyl, or NH₂, wherein (C₁-C₆)alkyl is optionally substituted with one or more substituents independently selected from halo, cyano, hydroxy, (C₁-C₆)alkoxy(C₁-C₆)alkyl, and (C₁-C₆)alkoxy.

In other embodiments or of the compound of Formula (II),
A is (C₆-C₁₀)aryl or 5- to 10-membered heteroaryl;
n is 0, 1, 2, 3, 4 or 5;
R¹ is independently for each occurrence (C₁-C₆)alkyl, (C₃-C₈)cycloalkyl, 4- to 7-membered heterocycloalkyl, (C₁-C₆)alkoxy, halo, cyano, formyl, carboxy, or alkoxycarbonyl, wherein (C₁-C₆)alkyl, (C₃-C₈)cycloalkyl, 4- to 7-membered heterocycloalkyl, and (C₁-C₆)alkoxy are each optionally substituted with one or more substituents independently selected from halo, hydroxy, cyano, amido, sulfonamido, (C₁-C₆)alkyl, (C₃-C₈)cycloalkyl, and (C₁-C₆)alkoxy; or
two vicinal occurrences of R¹ taken together with the atoms to which they are attached form a fused 5- to 7-membered cycloalkyl or 5- to 7-membered heterocycloalkyl ring, either of which is optionally substituted with one or more substituents independently selected from halo, hydroxy, cyano, amido, sulfonamido, (C₁-C₆)alkyl, (C₃-C₈)cycloalkyl, and (C₁-C₆)alkoxy;
Q is N or CR^{2c};
R^{2a}, R^{2b}, and R^{2c}, are each independently hydrogen, halo, cyano, or (C₁-C₆)alkyl, wherein (C₁-C₆)alkyl is optionally substituted with one or more substituents independently selected from halo, hydroxy, and (C₁-C₆)alkoxy;
R³ is (C₁-C₆)alkyl optionally substituted with one or more substituents independently selected from halo, hydroxy, and (C₁-C₆)alkoxy;
X and Z are independently NR⁴, C(R⁵)₂, or O;
R⁴ and R⁵ are independently for each occurrence hydrogen or (C₁-C₆)alkyl, wherein (C₁-C₆)alkyl is optionally substituted with one or more substituents independently selected from halo, hydroxy, and (C₁-C₆)alkoxy;
L is (C₁-C₆)alkylene optionally substituted with one or more substituents independently selected from halo, hydroxy, C₁-C₆)alkoxy, and cyano;
W^{a}—W^{b} represents:
   —C(R⁶)₂—C(R⁷)₂—,
   —O—C(R⁸)₂—,
   C(R⁸)₂—O—,
   C(R⁸)₂—NR⁹—, or
   —N═C(R¹⁰)—; and
R⁶, R⁷, R⁸, R⁹ and R¹⁰ are independently for each occurrence hydrogen or (C₁-C₆)alkyl, wherein (C₁-C₆)alkyl is optionally substituted with one or more substituents independently selected from halo, hydroxy, and (C₁-C₆)alkoxy.

Yet another aspect of the invention relates to a compound of Formula (III):

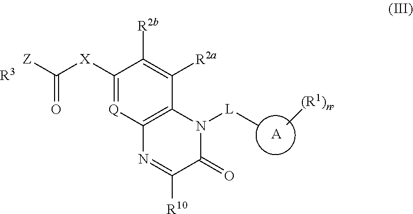

or a pharmaceutically acceptable salt thereof,
   A is (C₆-C₁₀)aryl, 5- to 10-membered heteroaryl, (C₃-C₈)cycloalkyl, or 4- to 7-membered heterocycloalkyl;
   n is 0, 1, 2, 3, 4, or 5;
   R¹ is independently for each occurrence (C₁-C₆)alkyl, (C₂-C₆)alkynyl, (C₂-C₆)alkenyl, (C₃-C₈)cycloalkyl, (C₃-C₈)cycloalkoxy, (C₆-C₁₀)aryl, (C₆-C₁₀)aryloxy, (C₆-C₁₀)aryl(C₁-C₆)alkyl, 4- to 7-membered heterocycloalkyl, (C₁-C₆)alkoxy, (C₁-C₆)alkoxy(C₁-C₆)alkyl, halo, cyano, amino, formyl, carboxy, or alkoxycarbonyl,
   wherein (C₁-C₆)alkyl, (C₂-C₆)alkynyl, (C₂-C₆)alkenyl, (C₃-C₈)cycloalkyl, C₃-C₈)cycloalkoxy, (C₆-C₁₀)aryl, (C₆-C₁₀)aryloxy, (C₆-C₁₀)aryl(C₁-C₆)alkyl, (C₁-C₆)alkoxy, (C₁-C₆)alkoxy(C₁-C₆)alkyl, and 4- to 7-membered heterocycloalkyl are each optionally substituted with one or more substituents independently selected from halo, hydroxy, cyano, amido, sulfonamido, (C₁-C₆)alkyl, (C₃-C₈)cycloalkyl, 4- to 7-membered heterocycloalkyl, 5- to 6-membered heteroaryl, (C₁-C₆)alkoxy, (C₁-C₆)haloalkoxy, (C₁-C₆)fluoroalkoxy, 5- to 6-membered heteroaryloxy, or
   two vicinal occurrences of R¹ taken together with the atoms to which they are attached form a fused 5- to 7-membered cycloalkyl, 5- to 7-membered heterocycloalkyl ring, 5- to 6-membered heteroaryl ring or phenyl ring, any of which is optionally substituted with one or more substituents independently selected from halo, hydroxy, cyano, amido, sulfonamido, (C₁-C₆)alkyl, (C₁-C₆)haloalkyl (C₃-C₈)cycloalkyl, and (C₁-C₆)alkoxy;
   Q is N or CR^{2c};
   R^{2a}, R^{2b}, and R^{2c}, are each independently hydrogen, halo, cyano, (C₁-C₆)alkyl, or (C₃-C₈)cycloalkyl, wherein (C₁-C₆)alkyl is optionally substituted with one or more substituents independently selected from halo, hydroxy, and (C₁-C₆)alkoxy;
   R³ is (C₁-C₆)alkyl, (C₃-C₈)cycloalkyl, (C₃-C₈)cycloalkyl(C₁-C₆)alkyl), (C₆-C₁₀)aryl, (C₆-C₁₀)aryl(C₁-C₆)alkyl), or 4- to 7-membered heterocycloalkyl; each of which is optionally substituted with one or more substituents independently selected from halo, cyano, (C₁-C₆)alkyl, (C₁-C₆)haloalkyl, hydroxy, (C₁-C₆)alkoxy(C₁-C₆)alkyl, and (C₁-C₆)alkoxy;
   X and Z are independently NR⁴, C(R⁵)₂, or O;
R⁴ and R⁵ independently for each occurrence hydrogen or (C₁-C₆)alkyl, wherein (C₁-C₆)alkyl is optionally substituted with one or more substituents independently selected from halo, hydroxy, and (C₁-C₆)alkoxy, or when Z is NR⁴, R³ and R⁴ taken together with the nitrogen to which they are attached form a 4- to 7-member heterocycloalkyl optionally substituted with one or more (C₁-C₆)alkyl or halo, or when Z is C(R⁵)₂, R³ and one R⁵ taken together with the carbon to which they are attached form a 4, (C₃-C₈)cycloalkyl optionally substituted with one or more (C₁-C₆)alkyl or halo;
   L is (C₁-C₆)alkylene optionally substituted with one or more substituents independently selected from halo, hydroxy, C₁-C₆)alkoxy, and cyano;
   R¹⁰ is hydrogen, (C₁-C₆)alkyl, or NH₂, wherein (C₁-C₆)alkyl is optionally substituted with one or more substituents independently selected from halo, cyano, hydroxy, (C₁-C₆)alkoxy(C₁-C₆)alkyl, and (C₁-C₆)alkoxy.

In some embodiments of the compound of Formula (III),
A is (C₆-C₁₀)aryl or 5- to 10-membered heteroaryl;
n is 0, 1, 2, 3, 4 or 5;
R¹ is independently for each occurrence (C₁-C₆)alkyl, (C₃-C₈)cycloalkyl, 4- to 7-membered heterocycloalkyl, (C₁-C₆)alkoxy, halo, cyano, formyl, carboxy, or alkoxycarbonyl, wherein (C₁-C₆)alkyl, (C₃-C₈)cycloalkyl, 4- to 7-membered heterocycloalkyl, and (C₁-C₆)alkoxy are each optionally substituted with one or more substituents independently selected from halo, hydroxy, cyano, amido, sulfonamido, (C₁-C₆)alkyl, (C₃-C₈)cycloalkyl, and (C₁-C₆)alkoxy; or
two vicinal occurrences of R¹ taken together with the atoms to which they are attached form a fused 5- to 7-membered cycloalkyl or 5- to 7-membered heterocycloalkyl ring, either of which is optionally substituted with one or more substituents independently selected from halo, hydroxy, cyano, amido, sulfonamido, (C₁-C₆)alkyl, (C₃-C₈)cycloalkyl, and (C₁-C₆)alkoxy;
Q is N or CR^{2c};
R^{2a}, R^{2b}, and R^{2c}, are each independently hydrogen, halo, cyano, (C₁-C₆)alkyl, wherein (C₁-C₆)alkyl is optionally substituted with one or more substituents independently selected from halo, hydroxy, and (C₁-C₆)alkoxy;
R³ is (C₁-C₆)alkyl optionally substituted with one or more substituents independently selected from halo, hydroxy, and (C₁-C₆)alkoxy;
X and Z are independently NR⁴, C(R⁵)₂, or O;
R⁴ and R⁵ are independently for each occurrence hydrogen or (C₁-C₆)alkyl, wherein (C₁-C₆)alkyl is optionally substituted with one or more substituents independently selected from halo, hydroxy, and (C₁-C₆)alkoxy;
L is (C₁-C₆)alkylene optionally substituted with one or more substituents independently selected from halo, hydroxy, C₁-C₆)alkoxy, and cyano; and
R¹⁰ is hydrogen or (C₁-C₆)alkyl, wherein (C₁-C₆)alkyl is optionally substituted with one or more substituents independently selected from halo, hydroxy, and (C₁-C₆)alkoxy.

In certain embodiments, A is phenyl.

In certain embodiments, A is a 5- to 7-membered heteroaryl comprising 1 or 2 nitrogen atoms.

In certain embodiments, A is pyridyl or diazolyl. thiazolyl, pyrazinyl, pyrimidinyl, while in other embodiments, A is pyridyl or diazolyl.

In some embodiments, A is wherein A is isoquinolinyl or indazolyl.

In other embodiments, A is cyclopentyl, cyclohexyl, or tetrahydropyranyl.

In certain embodiments, R¹ is independently for each occurrence (C₁-C₆)alkyl, (C₁-C₆)alkoxy, fluoro, chloro, or cyano, wherein (C₁-C₆)alkyl and (C₁-C₆)alkoxy are optionally substituted with one or more substituents independently selected from fluoro and hydroxyl.

In certain embodiments, at least one R¹ is fluoro, chloro, or cyano.

In certain embodiments, at least one R¹ is (C₁-C₆)alkyl optionally substituted with one or more substituents independently selected from hydroxyl and fluoro. In certain preferred embodiments, R¹ is 2,2,2-trifluoroethyl-1-hydroxyethyl. In other preferred embodiments, at least one R¹ is difluoromethyl, trifluoromethyl, 2,2,2-trifluoroethyl, 3,3,3-trifluropropyl, or 4,4,4-trifluorobutyl.

In certain embodiments, at least one R¹ is (C₁-C₆)alkoxy optionally substituted with at least one fluoro, preferably (C₁-C₃)alkoxy optionally substituted with at least one fluoro, more preferably difluoromethoxy or trifluoromethoxy. In other preferred embodiments, R¹ is methoxy or isopropoxy.

In certain embodiments, R¹ is methyl optionally substituted with hydroxy or one or more fluoro.

In certain embodiments, R¹ is (C₁-C₃)alkoxy optionally substituted with at least one fluoro.

In certain embodiments, at least one R¹ is (C₃-C₈)cycloalkyl, preferably cyclopropyl or cyclobutyl.

In certain embodiments, at least R¹ is (C₃-C₈)cycloalkoxy, preferably cyclopentyloxy, cyclohexyloxy.

In certain embodiments, R¹ is (C₂-C₆)alkynyl optionally substituted with hydroxy.

In certain embodiments, two vicinal occurrences of R¹ taken together with the atoms to which they are attached form a fused 5- to 7-membered cycloalkyl or 5- to 7-membered heterocycloalkyl ring, either of which is optionally substituted with one or more substituents independently selected from halo, hydroxy, cyano, amido, sulfonamido, (C₁-C₆)alkyl, (C₃-C₈)cycloalkyl, and (C₁-C₆)alkoxy.

In certain embodiments, A is phenyl comprising two vicinal occurrences of R¹, wherein the two vicinal occurrences of R¹, taken together with the atoms to which they are attached, form a fused 5- to 7-membered cycloalkyl or 5- to 7-membered heterocycloalkyl ring.

In certain embodiments, n is 2.

In certain embodiments, n is 1.

In certain embodiments, n is 0.

In certain embodiments, Q is CR^{2c}.

In certain embodiments, Q is N.

In certain embodiments, R^{2a}, R^{2b}, and R^{2c} are each hydrogen.

In certain embodiments, at least one of R^{2a}, R^{2b}, and R^{2c} is fluoro, chloro, or cyano.

In certain embodiments, R³ is (C₁-C₆)alkyl optionally substituted with hydroxy.

In certain preferred embodiments, R³ is t-butyl.

In certain embodiments, R³ is (C₃-C₈)cycloalkyl optionally substituted with one or more (C₁-C₆)alkyl, (C₁-C₆)fluoroalkyl, fluoro, or (C₁-C₆)alkoxy(C₁-C₆)alkyl. In more particular embodiments, the (C₃-C₈)cycloalkyl is cyclobutyl, cyclopentyl, cyclohexyl, bicyclo[1.1.1]pentanyl, bicyclo[2.2.1]heptanyl,bicycle[2.1.1]hexanyl, or bicyclo[3.1.0]hexanyl. In further particular embodiments, the (C₃-C₈)cycloalkyl is optionally substituted with one or more substituents selected from fluoro, trifluoromethyl, methoxymethyl, methyl, and hydroxyl.

In certain embodiments, R³ is 4- to 7-membered heterocycloalkyl optionally substituted with one or more substituents selected from (C₁-C₆)alkyl, (C₁-C₆)fluoroalkyl, or fluoro. In more particular embodiments, the 4- to 7-membered heterocycloalkyl is etrahydropyranyl or oxetanyl.

In some embodiments, Z is NR⁴, R³ and R⁴ taken together with the nitrogen to which they are attached form a 4- to 7-member heterocycloalkyl optionally substituted with one or more (C₁-C₆)alkyl or halo. In some embodiments, the 4- to 7 membered heterocycloalkyl is azetidine or pyrrolidine,

In certain embodiments, X is NR⁴ or O.

In certain embodiments, X is NH.

In certain embodiments, Z is NR⁴, or O.

In certain embodiments, Z is NH.

In certain embodiments, one and only one of X and Z is CH₂.

In certain embodiments, X and Z are each NH.

In certain embodiments, L is unsubstituted (C₁-C₆)alkylene.

In certain embodiments, L is (C₁-C₆)alkylene substituted with hydroxy.

In certain embodiments, L is (C₁-C₆)alkylene substituted with 1 to 3 halo atoms.

In certain embodiments, L is —CH₂—, —CH(CH₃)—, —CH(CH₂CH₃)—, or —CH(CH₂OH)—.

In certain embodiments, W^{a}—W^{b} represents:
—C(R⁶)₂—C(R⁷)₂—.

In certain embodiments, R⁶ is (C₁-C₆)alkyl, wherein (C₁-C₆)alkyl is optionally substituted with one or more substituents independently selected from halo, hydroxy, and (C₁-C₆)alkoxy.

In certain embodiments, at least one R⁷ is (C₁-C₆)alkyl, wherein (C₁-C₆)alkyl is optionally substituted with one or more substituents independently selected from halo, hydroxy, (C₁-C₆)alkoxy, and cyano.

In certain embodiments, each R⁶ and each R⁷ is hydrogen.

In certain embodiments, W^{a}—W^{b} represents:
—O—C(R⁸)₂—.

In certain embodiments, R⁸ is hydrogen.

In certain embodiments, one R⁸(C₁-C₆)alkyl, preferably methyl; and one R⁸ is hydrogen.

In certain embodiments, W^{a}—W^{b} represents:
—C(R⁸)₂—O—, where each R⁸ is hydrogen.

In certain embodiments, W^{a}—W^{b} represents:
—C(R⁸)₂—NR⁹—.

In certain embodiments, each R⁸ is hydrogen.

In certain embodiments, R⁹ is (C₁-C₆)alkyl, preferably methyl.

In certain embodiments, W^{a}—W^{b} represents:
—N═C(R¹⁰)—.

In certain embodiments, R¹⁰ is hydrogen.

In certain embodiments, R¹⁰ is (C₁-C₆)alkyl.

In certain embodiments, R¹⁰ is methyl.

In certain embodiments, a compound is selected from Table 1:

TABLE 1

| | | |
|---|---|---|
| 1 | 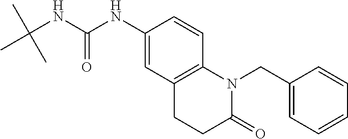 [SMILES: CC(C)(C)NC(=O)Nc1ccc2c(c1)CCC(=O)N2Cc1ccccc1] | 1-(1-benzyl-2-oxo-1,2,3,4-tetrahydroquinolin-6-yl)-3- (tert-butyl)urea |
| 2 | 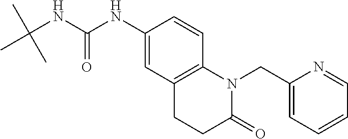 [SMILES: CC(C)(C)NC(=O)Nc1ccc2c(c1)CCC(=O)N2Cc1ccccn1] | 1-(tert-butyl)-3-(2-oxo-1-(pyridin-2-ylmethyl)-1,2,3,4- tetrahydroquinolin-6-yl)urea |
| 3 | 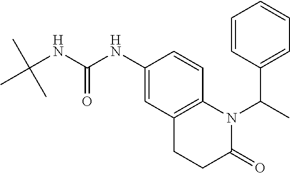 [SMILES: CC(c1ccccc1)N1C(=O)CCc2cc(NC(=O)NC(C)(C)C)ccc21] | 1-(tert-butyl)-3-(2-oxo-1-(1-phenylethyl)-1,2,3,4- tetrahydroquinolin-6-yl)urea |
| 4 | 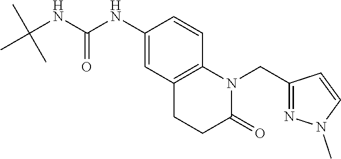 [SMILES: Cn1ccc(CN2C(=O)CCc3cc(NC(=O)NC(C)(C)C)ccc32)n1] | 1-(tert-butyl)-3-(1-((1-methyl-1H-pyrazol-3-yl)methyl)-2- oxo-1,2,3,4-tetrahydroquinolin-6-yl)urea |
| 5 | 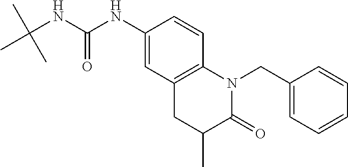 [SMILES: CC1Cc2cc(NC(=O)NC(C)(C)C)ccc2N(Cc2ccccc2)C1=O] | 1-(1-benzyl-3-methyl-2-oxo-1,2,3,4-tetrahydroquinolin-6- yl)-3-(tert-butyl)urea |
| 6 | 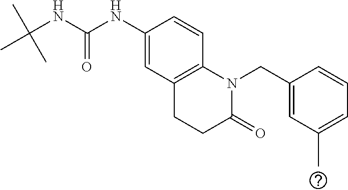 [SMILES: Cc1cccc(CN2C(=O)CCc3cc(NC(=O)NC(C)(C)C)ccc32)c1] | 1-(tert-butyl)-3-(1-(3-chlorobenzyl)-2-oxo-1,2,3,4- tetrahydroquinolin-6-yl)urea |
| 7 | 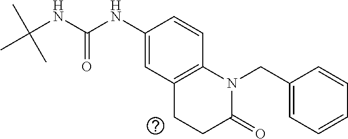 [SMILES: CC(C)(C)NC(=O)Nc1ccc2c(c1)CCC(=O)N2Cc1ccccc1] | 1-(4-benzyl-3-oxo-3,4-dihydro-2H-benzo[b][1,4]oxazin- 7-yl)-3-(tert-butyl)urea |
| 8 | 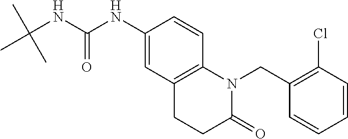 [SMILES: CC(C)(C)NC(=O)Nc1ccc2c(c1)CCC(=O)N2Cc1ccccc1Cl] | 1-(tert-butyl)-3-(1-(2-chlorobenzyl)-2-oxo-1,2,3,4- tetrahydroquinolin-6-yl)urea |
| 9 | 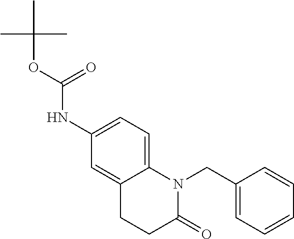 [SMILES: CC(C)(C)OC(=O)Nc1ccc2c(c1)CCC(=O)N2Cc1ccccc1] | tert-butyl (1-benzyl-2-oxo-1,2,3,4-tetrahydroquinolin-6- yl)carbamate |
| 10 | 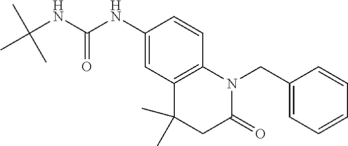 [SMILES: CC(C)(C)NC(=O)Nc1ccc2c(c1)C(C)(C)CC(=O)N2Cc1ccccc1] | 1-(1-benzyl-4,4-dimethyl-2-oxo-1,2,3,4- tetrahydroquinolin-6-yl)-3-(tert-butyl)urea |
| 11 | 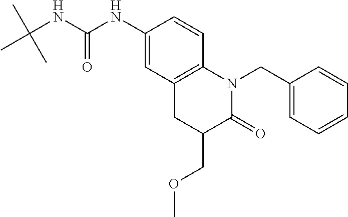 [SMILES: COCC1Cc2cc(NC(=O)NC(C)(C)C)ccc2N(Cc2ccccc2)C1=O] | 1-(1-benzyl-3-(methoxymethyl)-2-oxo-1,2,3,4- tetrahydroquinolin-6-yl)-3-(tert-butyl)urea |
| 12 | 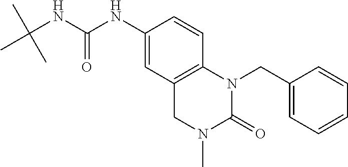 [SMILES: CN1Cc2cc(NC(=O)NC(C)(C)C)ccc2N(Cc2ccccc2)C1=O] | 1-(1-benzyl-3-methyl-2-oxo-1,2,3,4-tetrahydroquinazolin- 6-yl)-3-(tert-butyl)urea |
| 13 | 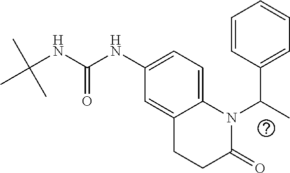 [SMILES: CC(c1ccccc1)N1C(=O)CCc2cc(NC(=O)NC(C)(C)C)ccc21] | (S)-1-(tert-butyl)-3-(2-oxo-1-(1-phenylethyl)-1,2,3,4- tetrahydroquinolin-6-yl)urea |
| 14 | 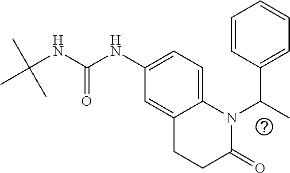 [SMILES: CC(c1ccccc1)N1C(=O)CCc2cc(NC(=O)NC(C)(C)C)ccc21] | (R)-1-(tert-butyl)-3-(2-oxo-1-(1-phenylethyl)-1,2,3,4- tetrahydroquinolin-6-yl)urea |
| 15 | 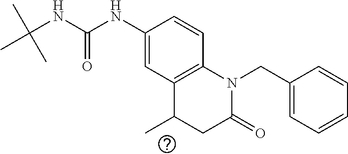 [SMILES: CC1CC(=O)N(Cc2ccccc2)c2ccc(NC(=O)NC(C)(C)C)cc21] | 1-(1-benzyl-4-methyl-2-oxo-1,2,3,4-tetrahydroquinolin-6- yl)-3-(tert-butyl)urea |
| 16 | 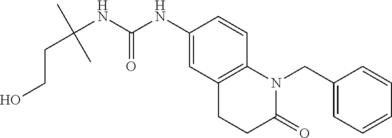 [SMILES: CC(C)(CCO)NC(=O)Nc1ccc2c(c1)CCC(=O)N2Cc1ccccc1] | 1-(1-benzyl-2-oxo-1,2,3,4-tetrahydroquinolin-6-yl)-3-(4- hydroxy-2-methylbutan-2-yl)urea |
| 17 | 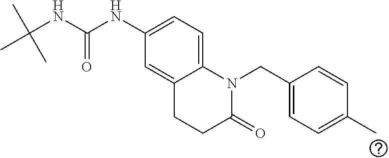 [SMILES: Cc1ccc(CN2C(=O)CCc3cc(NC(=O)NC(C)(C)C)ccc32)cc1] | 1-(tert-butyl)-3-(1-(4-cyanobenzyl)-2-oxo-1,2,3,4- tetrahydroquinolin-6-yl)urea |
| 18 | 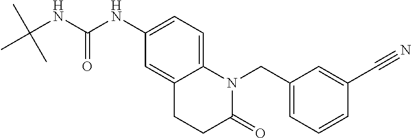 [SMILES: CC(C)(C)NC(=O)Nc1ccc2c(c1)CCC(=O)N2Cc1cccc(C#N)c1] | 1-(tert-butyl)-3-(1-(3-cyanobenzyl)-2-oxo-1,2,3,4- tetrahydroquinolin-6-yl)urea |
| 19 | 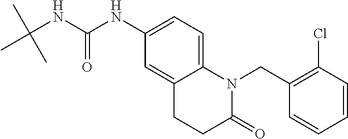 [SMILES: CC(C)(C)NC(=O)Nc1ccc2c(c1)CCC(=O)N2Cc1ccccc1Cl] | 1-(tert-butyl)-3-(1-(2-fluorobenzyl)-2-oxo-1,2,3,4- tetrahydroquinolin-6-yl)urea |
| 20 | 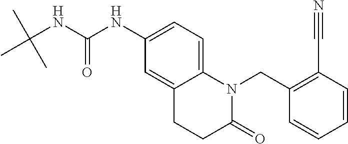 [SMILES: CC(C)(C)NC(=O)Nc1ccc2c(c1)CCC(=O)N2Cc1ccccc1C#N] | 1-(tert-butyl)-3-(1-(2-cyanobenzyl)-2-oxo-1,2,3,4- tetrahydroquinolin-6-yl)urea |
| 21 | 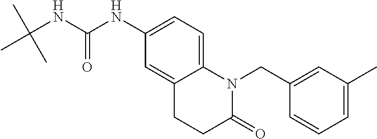 [SMILES: Cc1cccc(CN2C(=O)CCc3cc(NC(=O)NC(C)(C)C)ccc32)c1] | 1-(tert-butyl)-3-(1-(3-methylbenzyl)-2-oxo-1,2,3,4- tetrahydroquinolin-6-yl)urea |
| 22 | 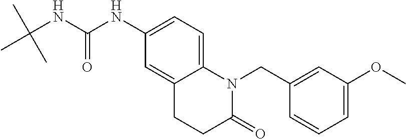 [SMILES: COc1cccc(CN2C(=O)CCc3cc(NC(=O)NC(C)(C)C)ccc32)c1] | 1-(tert-butyl)-3-(1-(3-methoxybenzyl)-2-oxo-1,2,3,4- tetrahydroquinolin-6-yl)urea |
| 23 | 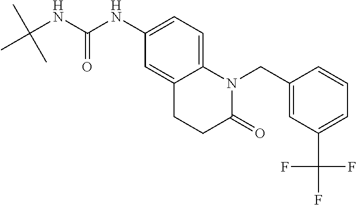 [SMILES: CC(C)(C)NC(=O)Nc1ccc2c(c1)CCC(=O)N2Cc1cccc(C(F)(F)F)c1] | 1-(tert-butyl)-3-(2-oxo-1-(3-(trifluoromethyl)benzyl)- 1,2,3,4-tetrahydroquinolin-6-yl)urea |
| 24 | 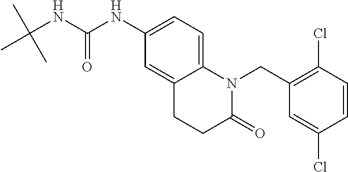 [SMILES: CC(C)(C)NC(=O)Nc1ccc2c(c1)CCC(=O)N2Cc1cc(Cl)ccc1Cl] | 1-(tert-butyl)-3-(1-(2,5-dichlorobenzyl)-2-oxo-1,2,3,4- tetrahydroquinolin-6-yl)urea |
| 25 | 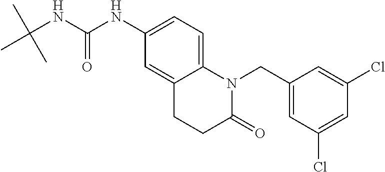 [SMILES: CC(C)(C)NC(=O)Nc1ccc2c(c1)CCC(=O)N2Cc1cc(Cl)cc(Cl)c1] | 1-(tert-butyl)-3-(1-(3,5-dichlorobenzyl)-2-oxo-1,2,3,4- tetrahydroquinolin-6-yl)urea |
| 26 | 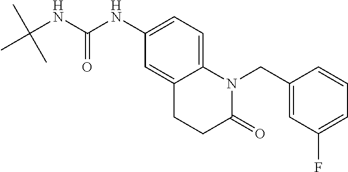 [SMILES: CC(C)(C)NC(=O)Nc1ccc2c(c1)CCC(=O)N2Cc1cccc(F)c1] | 1-(tert-butyl)-3-(1-(3-fluorobenzyl)-2-oxo-1,2,3,4- tetrahydroquinolin-6-yl)urea |
| 27 | 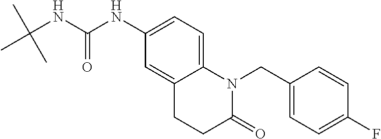 [SMILES: CC(C)(C)NC(=O)Nc1ccc2c(c1)CCC(=O)N2Cc1ccc(F)cc1] | 1-(tert-butyl)-3-(1-(4-fluorobenzyl)-2-oxo-1,2,3,4- tetrahydroquinolin-6-yl)urea |
| 28 | 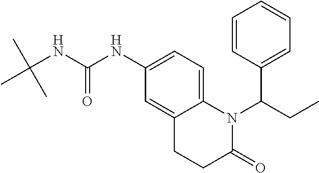 [SMILES: CCC(c1ccccc1)N1C(=O)CCc2cc(NC(=O)NC(C)(C)C)ccc21] | 1-(tert-butyl)-3-(2-oxo-1-(1-phenylpropyl)-1,2,3,4- tetrahydroquinolin-6-yl)urea |
| 29 | 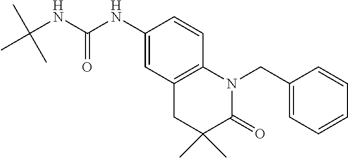 [SMILES: CC(C)(C)NC(=O)Nc1ccc2c(c1)CC(C)(C)C(=O)N2Cc1ccccc1] | 1-(1-benzyl-3,3-dimethyl-2-oxo-1,2,3,4- tetrahydroquinolin-6-yl)-3-(tert-butyl)urea |
| 30 | 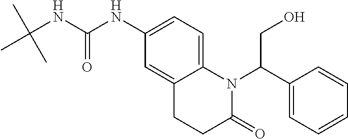 [SMILES: CC(C)(C)NC(=O)Nc1ccc2c(c1)CCC(=O)N2C(CO)c1ccccc1] | 1-(tert-butyl)-3-(1-(2-hydroxy-1-phenylethyl)-2-oxo- 1,2,3,4-tetrahydroquinolin-6-yl)urea |
| 31 | 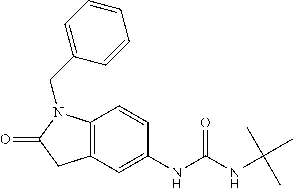 [SMILES: CC(C)(C)NC(=O)Nc1ccc2c(c1)CC(=O)N2Cc1ccccc1] | 1-(1-benzyl-2-oxoindolin-5-yl)-3-(tert-butyl)urea |
| 32 | 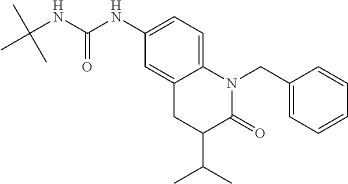 [SMILES: CC(C)C1Cc2cc(NC(=O)NC(C)(C)C)ccc2N(Cc2ccccc2)C1=O] | 1-(1-benzyl-3-isopropyl-2-oxo-1,2,3,4- tetrahydroquinolin-6-yl)-3-(tert-butyl)urea |
| 33 | 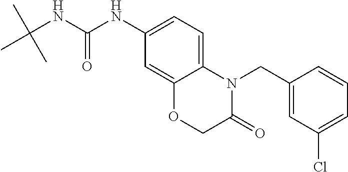 [SMILES: CC(C)(C)NC(=O)Nc1ccc2c(c1)OCC(=O)N2Cc1cccc(Cl)c1] | 1-(tert-butyl)-3-(4-(3-chlorobenzyl)-3-oxo-3,4-dihydro- 2H-benzo[b][1,4]oxazin-7-yl)urea |
| 34 | 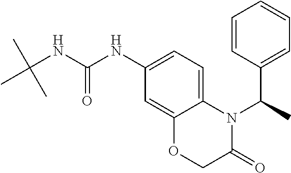 [SMILES: C[C@H](c1ccccc1)N1C(=O)COc2cc(NC(=O)NC(C)(C)C)ccc21] | (R)-1-(tert-butyl)-3-(3-oxo-4-(1-phenylethyl)-3,4- dihydro-2H-benzo[b][1,4]oxazin-7-yl)urea |
| 35 | 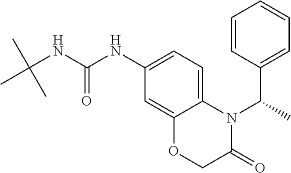 [SMILES: C[C@@H](c1ccccc1)N1C(=O)COc2cc(NC(=O)NC(C)(C)C)ccc21] | (S)-1-(tert-butyl)-3-(3-oxo-4-(1-phenylethyl)-3,4-dihydro- 2H-benzo[b][1,4]oxazin-7-yl)urea |
| | C₂₁H₂₅N₃O₃ | |
| 36 | 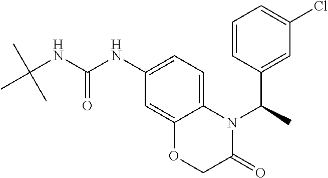 [SMILES: C[C@H](c1cccc(Cl)c1)N1C(=O)COc2cc(NC(=O)NC(C)(C)C)ccc21] | (R)-1-(tert-butyl)-3-(4-(1-(3-chlorophenyl)ethyl)-3-oxo- 3,4-dihydro-2H-benzo[b][1,4]oxazin-7-yl)urea |
| 37 | 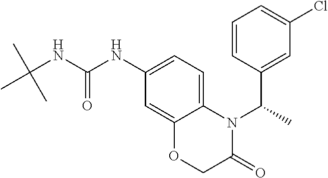 [SMILES: C[C@@H](c1cccc(Cl)c1)N1C(=O)COc2cc(NC(=O)NC(C)(C)C)ccc21] | (S)-1-(tert-butyl)-3-(4-(1-(3-chlorophenyl)ethyl)-3-oxo- 3,4-dihydro-2H-benzo[b][1,4]oxazin-7-yl)urea |
| 38 | 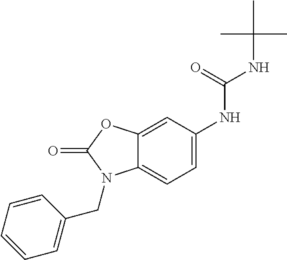 [SMILES: CC(C)(C)NC(=O)Nc1ccc2c(c1)oc(=O)n2Cc1ccccc1] | 1-(3-benzyl-2-oxo-2,3-dihydrobenzo[d]oxazol-6-yl)-3- (tert-butyl)urea |
| 39 | 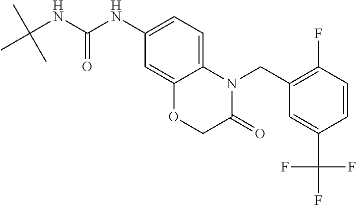 [SMILES: CC(C)(C)NC(=O)Nc1ccc2c(c1)OCC(=O)N2Cc1cc(C(F)(F)F)ccc1F] | 1-(tert-butyl)-3-(1-(2-fluoro-5-(trifluoromethyl)benzyl)-2- oxo-1,2,3,4-tetrahydroquinolin-6-yl)urea |
| 40 | 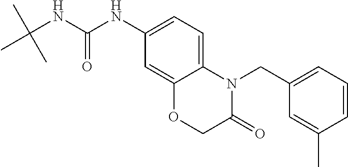 [SMILES: Cc1cccc(CN2C(=O)COc3cc(NC(=O)NC(C)(C)C)ccc32)c1] | 1-(tert-butyl)-3-(4-(3-methylbenzyl)-3-oxo-3,4-dihydro- 2H-benzo[b][1,4]oxazin-7-yl)urea |
| 41 | 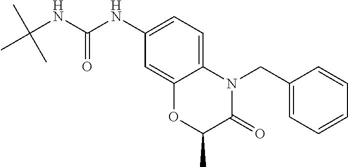 [SMILES: C[C@H]1Oc2cc(NC(=O)NC(C)(C)C)ccc2N(Cc2ccccc2)C1=O] | (R)-1-(4-benzyl-2-methyl-3-oxo-3,4-dihydro-2H- benzo[b][1,4]oxazin-7-yl)-3-(tert-butyl)urea |
| 42 | 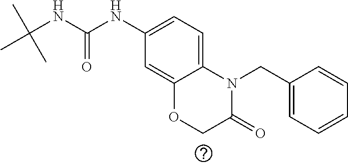 [SMILES: CC(C)(C)NC(=O)Nc1ccc2c(c1)OCC(=O)N2Cc1ccccc1] | (S)-1-(4-benzyl-2-methyl-3-oxo-3,4-dihydro-2H- benzo[b][1,4]oxazin-7-yl)-3-(tert-butyl)urea |
| 43 | 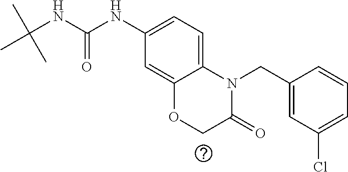 [SMILES: CC(C)(C)NC(=O)Nc1ccc2c(c1)OCC(=O)N2Cc1cccc(Cl)c1] | (S)-1-(tert-butyl)-3-(4-(3-chlorobenzyl)-2-methyl-3-oxo- 3,4-dihydro-2H-benzo[b][1,4]oxazin-7-yl)urea |
| 44 | 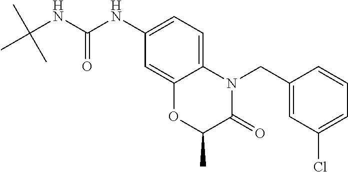 [SMILES: C[C@H]1Oc2cc(NC(=O)NC(C)(C)C)ccc2N(Cc2cccc(Cl)c2)C1=O] | (R)-1-(tert-butyl)-3-(4-(3-chlorobenzyl)-2-methyl-3-oxo- 3,4-dihydro-2H-benzo[b][1,4]oxazin-7-yl)urea |
| 45 | 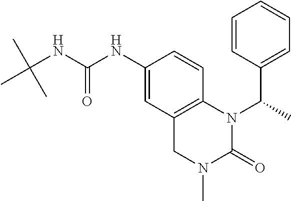 [SMILES: C[C@@H](c1ccccc1)N1C(=O)N(C)Cc2cc(NC(=O)NC(C)(C)C)ccc21] | (S)-1-(tert-butyl)-3-(3-methyl-2-oxo-1-(1-phenylethyl)- 1,2,3,4-tetrahydroquinazolin-6-yl)urea |
| 46 | 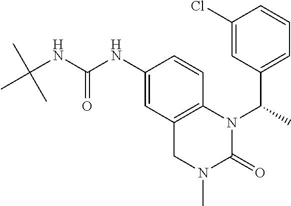 [SMILES: C[C@@H](c1cccc(Cl)c1)N1C(=O)N(C)Cc2cc(NC(=O)NC(C)(C)C)ccc21] | (S)-1-(tert-butyl)-3-(1-(1-(3-chlorophenyl)ethyl)-3- methyl-2-oxo-1,2,3,4-tetrahydroquinazolin-6-yl)urea |
| 47 | 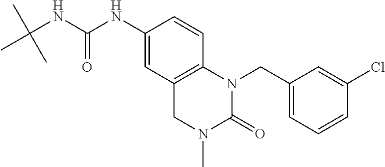 [SMILES: CN1Cc2cc(NC(=O)NC(C)(C)C)ccc2N(Cc2cccc(Cl)c2)C1=O] | 1-(tert-butyl)-3-(1-(3-chlorobenzyl)-3-methyl-2-oxo- 1,2,3,4-tetrahydroquinazolin-6-yl)urea |
| 48 | 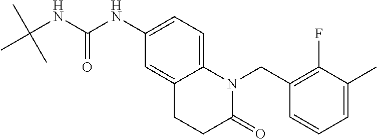 [SMILES: Cc1cccc(CN2C(=O)CCc3cc(NC(=O)NC(C)(C)C)ccc32)c1F] | 1-(tert-butyl)-3-(1-(2-fluoro-3-methylbenzyl)-2-oxo- 1,2,3,4-tetrahydroquinolin-6-yl)urea |
| 49 | 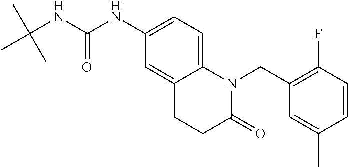 [SMILES: Cc1ccc(F)c(CN2C(=O)CCc3cc(NC(=O)NC(C)(C)C)ccc32)c1] | 1-(tert-butyl)-3-(1-(2-fluoro-5-methylbenzyl)-2-oxo- 1,2,3,4-tetrahydroquinolin-6-yl)urea |
| 50 | 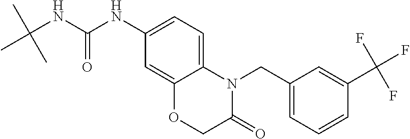 [SMILES: CC(C)(C)NC(=O)Nc1ccc2c(c1)OCC(=O)N2Cc1cccc(C(F)(F)F)c1] | 1-(tert-butyl)-3-(3-oxo-4-(3-(trifluoromethyl)benzyl)-3,4- dihydro-2H-benzo[b][1,4]oxazin-7-yl)urea |
| 51 | 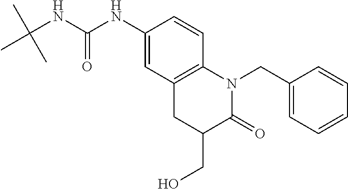 [SMILES: CC(C)(C)NC(=O)Nc1ccc2c(c1)CC(CO)C(=O)N2Cc1ccccc1] | 1-(1-benzyl-3-(hydroxymethyl)-2-oxo-1,2,3,4- tetrahydroquinolin-6-yl)-3-(tert-butyl)urea |
| 52 | 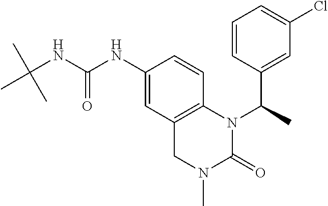 [SMILES: C[C@H](c1cccc(Cl)c1)N1C(=O)N(C)Cc2cc(NC(=O)NC(C)(C)C)ccc21] | (R)-1-(tert-butyl)-3-(1-(1-(3-chlorophenyl)ethyl)-3- methyl-2-oxo-1,2,3,4-tetrahydroquinazolin-6-yl)urea |
| 53 | 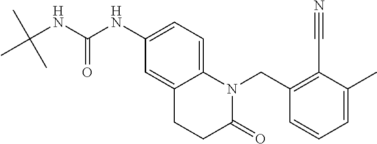 [SMILES: Cc1cccc(CN2C(=O)CCc3cc(NC(=O)NC(C)(C)C)ccc32)c1C#N] | 1-(tert-butyl)-3-(1-(2-cyano-3-methylbenzyl)-2-oxo- 1,2,3,4-tetrahydroquinolin-6-yl)urea |
| 54 | 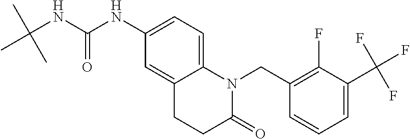 [SMILES: CC(C)(C)NC(=O)Nc1ccc2c(c1)CCC(=O)N2Cc1cccc(C(F)(F)F)c1F] | 1-(tert-butyl)-3-(1-(2-fluoro-3-(trifluoromethyl)benzyl)-2- oxo-1,2,3,4-tetrahydroquinolin-6-yl)urea |
| 55 | 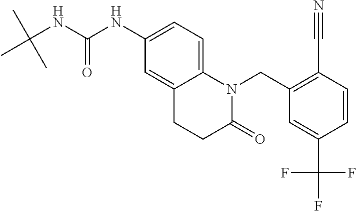 [SMILES: CC(C)(C)NC(=O)Nc1ccc2c(c1)CCC(=O)N2Cc1cc(C(F)(F)F)ccc1C#N] | 1-(tert-butyl)-3-(1-(2-cyano-5-(trifluoromethyl)benzyl)-2- oxo-1,2,3,4-tetrahydroquinolin-6-yl)urea |
| 56 | 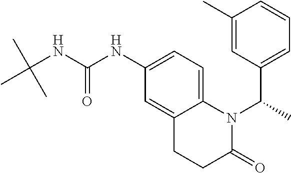 [SMILES: Cc1cccc([C@H](C)N2C(=O)CCc3cc(NC(=O)NC(C)(C)C)ccc32)c1] | (S)-1-(tert-butyl)-3-(2-oxo-1-(1-(m-tolyl)ethyl)-1,2,3,4- tetrahydroquinolin-6-yl)urea |
| 57 | 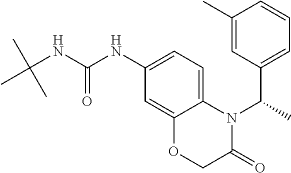 [SMILES: Cc1cccc([C@H](C)N2C(=O)COc3cc(NC(=O)NC(C)(C)C)ccc32)c1] | (S)-1-(tert-butyl)-3-(3-oxo-4-(1-(m-tolyl)ethyl)-3,4- dihydro-2H-benzo[b][1,4]oxazin-7-yl)urea |
| 58 | 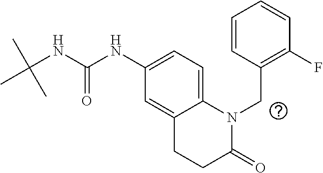 [SMILES: CC(C)(C)NC(=O)Nc1ccc2c(c1)CCC(=O)N2Cc1ccccc1F] | (S)-1-(tert-butyl)-3-(1-(1-(2-fluorophenyl)ethyl)-2-oxo- 1,2,3,4-tetrahydroquinolin-6-yl)urea |
| 59 | 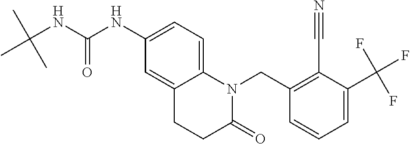 [SMILES: CC(C)(C)NC(=O)Nc1ccc2c(c1)CCC(=O)N2Cc1cccc(C(F)(F)F)c1C#N] | 1-(tert-butyl)-3-(1-(2-cyano-3-(trifluoromethyl)benzyl)-2- oxo-1,2,3,4-tetrahydroquinolin-6-yl)urea |
| 60 | 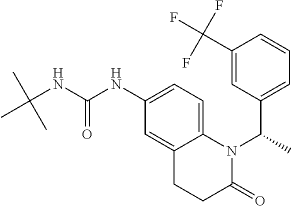 [SMILES: C[C@@H](c1cccc(C(F)(F)F)c1)N1C(=O)CCc2cc(NC(=O)NC(C)(C)C)ccc21] | (S)-1-(tert-butyl)-3-(2-oxo-1-(1-(3- (trifluoromethyl)phenyl)ethyl)-1,2,3,4-tetrahydroquinolin- 6-yl)urea |
| 61 | 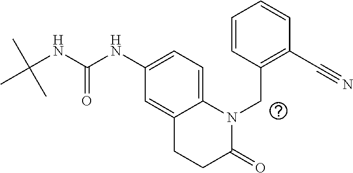 [SMILES: CC(C)(C)NC(=O)Nc1ccc2c(c1)CCC(=O)N2Cc1ccccc1C#N] | (S)-1-(tert-butyl)-3-(1-(1-(2-cyanophenyl)ethyl)-2-oxo- 1,2,3,4-tetrahydroquinolin-6-yl)urea |
| 62 | 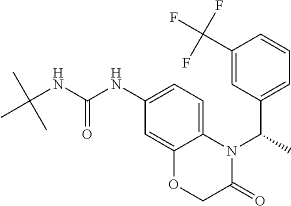 [SMILES: C[C@@H](c1cccc(C(F)(F)F)c1)N1C(=O)COc2cc(NC(=O)NC(C)(C)C)ccc21] | (S)-1-(tert-butyl)-3-(3-oxo-4-(1-(3- (trifluoromethyl)phenyl)ethyl)-3,4-dihydro-2H- benzo[b][1,4]oxazin-7-yl)urea |
| 63 | 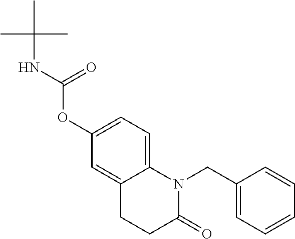 [SMILES: CC(C)(C)NC(=O)Oc1ccc2c(c1)CCC(=O)N2Cc1ccccc1] | 1-benzyl-2-oxo-1,2,3,4-tetrahydroquinolin-6-yl tert- butylcarbamate |
| 64 | 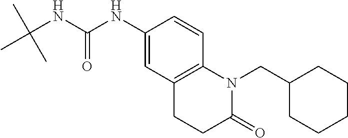 [SMILES: CC(C)(C)NC(=O)Nc1ccc2c(c1)CCC(=O)N2CC1CCCCC1] | 1-(tert-butyl)-3-(1-(cyclohexylmethyl)-2-oxo-1,2,3,4- tetrahydroquinolin-6-yl)urea |
| 65 | 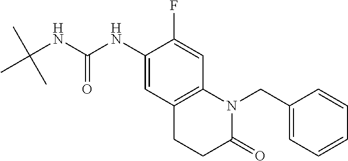 [SMILES: CC(C)(C)NC(=O)Nc1cc2c(cc1F)N(Cc1ccccc1)C(=O)CC2] | 1-(1-benzyl-7-fluoro-2-oxo-1,2,3,4-tetrahydroquinolin-6- yl)-3-(tert-butyl)urea |
| 66 | 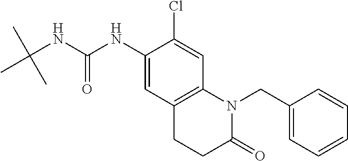 [SMILES: CC(C)(C)NC(=O)Nc1cc2c(cc1Cl)N(Cc1ccccc1)C(=O)CC2] | 1-(1-benzyl-7-chloro-2-oxo-1,2,3,4-tetrahydroquinolin-6- yl)-3-(tert-butyl)urea |
| 67 | 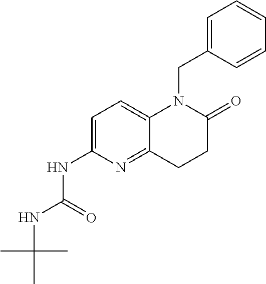 [SMILES: CC(C)(C)NC(=O)Nc1ccc2c(n1)CCC(=O)N2Cc1ccccc1] | 1-(5-benzyl-6-oxo-5,6,7,8-tetrahydro-1,5-naphthyridin-2- yl)-3-(tert-butyl)urea |
| 68 | 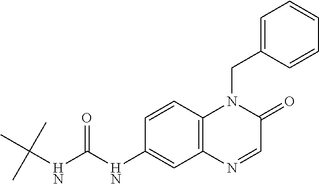 [SMILES: CC(C)(C)NC(=O)Nc1ccc2c(c1)ncc(=O)n2Cc1ccccc1] | 1-(1-benzyl-2-oxo-1,2-dihydroquinoxalin-6-yl)-3-(tert- butyl)urea |
| 69 | 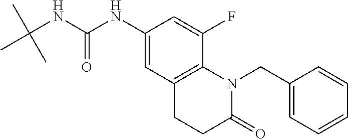 [SMILES: CC(C)(C)NC(=O)Nc1cc(F)c2c(c1)CCC(=O)N2Cc1ccccc1] | 1-(1-benzyl-8-fluoro-2-oxo-1,2,3,4-tetrahydroquinolin-6- yl)-3-(tert-butyl)urea; and |
| 70 | 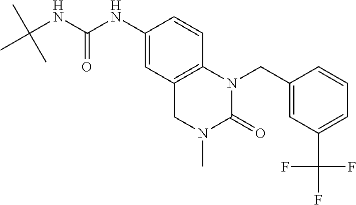 [SMILES: CN1Cc2cc(NC(=O)NC(C)(C)C)ccc2N(Cc2cccc(C(F)(F)F)c2)C1=O] | 1-(tert-butyl)-3-(3-methyl-2-oxo-1-(3- (trifluoromethyl)benzyl)-1,2,3,4-tetrahydroquinazolin-6- yl)urea |
| 71 | 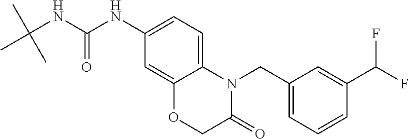 [SMILES: CC(C)(C)NC(=O)Nc1ccc2c(c1)OCC(=O)N2Cc1cccc(C(F)F)c1] | 1-(tert-butyl)-3-(4-(3-(difluoromethyl)benzyl)-3-oxo-3,4- dihydro-2H-benzo[b][1,4]oxazin-7-yl)urea |
| | C₂₁H₂₃F₂N₃O₃ | |
| 72 | 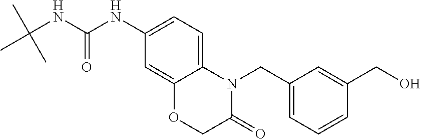 [SMILES: CC(C)(C)NC(=O)Nc1ccc2c(c1)OCC(=O)N2Cc1cccc(CO)c1] | 1-(tert-butyl)-3-(4-(3-(hydroxymethyl)benzyl)-3-oxo-3,4- dihydro-2H-benzo[b][1,4]oxazin-7-yl)urea |
| | C₂₁H₂₅N₃O₄ | |
| 73 | 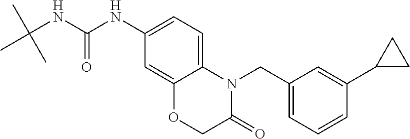 [SMILES: CC(C)(C)NC(=O)Nc1ccc2c(c1)OCC(=O)N2Cc1cccc(C2CC2)c1] | 1-(tert-butyl)-3-(4-(3-cyclopropylbenzyl)-3-oxo-3,4- dihydro-2H-benzo[b][1,4]oxazin-7-yl)urea |
| | C₂₃H₂₇N₃O₃ | |
| 74 | 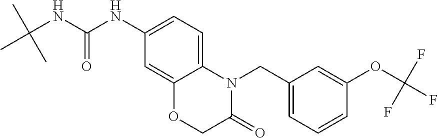 [SMILES: CC(C)(C)NC(=O)Nc1ccc2c(c1)OCC(=O)N2Cc1cccc(OC(F)(F)F)c1] | 1-(tert-butyl)-3-(3-oxo-4-(3-(trifluoromethoxy)benzyl)- 3,4-dihydro-2H-benzo[b][1,4]oxazin-7-yl)urea |
| | C₂₁H₂₂F₃N₃O₄ | |
| 75 | 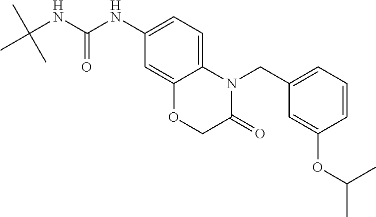 [SMILES: CC(C)Oc1cccc(CN2C(=O)COc3cc(NC(=O)NC(C)(C)C)ccc32)c1] | 1-(tert-butyl)-3-(4-(3-isopropoxybenzyl)-3-oxo-3,4- dihydro-2H-benzo[b][1,4]oxazin-7-yl)urea |
| | C₂₃H₂₉N₃O₄ | |
| 76 | 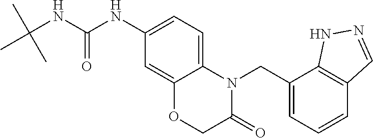 [SMILES: CC(C)(C)NC(=O)Nc1ccc2c(c1)OCC(=O)N2Cc1cccc2cn[nH]c12] | 1-(4-((1H-indazol-7-yl)methyl)-3-oxo-3,4-dihydro-2H- benzo[b][1,4]oxazin-7-yl)-3-(tert-butyl)urea |
| | C₂₁H₂₃N₅O₃ | |
| 77 | 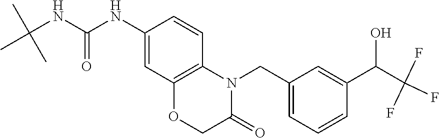 [SMILES: CC(C)(C)NC(=O)Nc1ccc2c(c1)OCC(=O)N2Cc1cccc(C(O)C(F)(F)F)c1] | 1-(tert-butyl)-3-(3-oxo-4-(3-(2,2,2-trifluoro-1- hydroxyethyl)benzyl)-3,4-dihydro-2H- benzo[b][1,4]oxazin-7-yl)urea |
| | C₂₂H₂₄F₃N₃O₄ | |
| 78 | 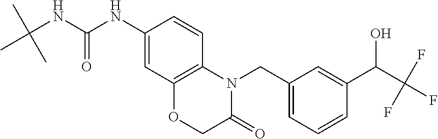 [SMILES: CC(C)(C)NC(=O)Nc1ccc2c(c1)OCC(=O)N2Cc1cccc(C(O)C(F)(F)F)c1] | (R)-1-(tert-butyl)-3-(3-oxo-4-(3-(2,2,2-trifluoro-1- hydroxyethyl)benzyl)-3,4-dihydro-2H- benzo[b][1,4]oxazin-7-yl)urea |
| | C₂₂H₂₄F₃N₃O₄ | |
| 79 | 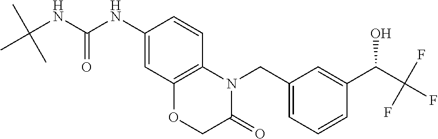 [SMILES: CC(C)(C)NC(=O)Nc1ccc2c(c1)OCC(=O)N2Cc1cccc([C@H](O)C(F)(F)F)c1] | (S)-1-(tert-butyl)-3-(3-oxo-4-(3-(2,2,2-trifluoro-1- hydroxyethyl)benzyl)-3,4-dihydro-2H- benzo[b][1,4]oxazin-7-yl)urea |
| | C₂₂H₂₄F₃N₃O₄ | |
| 80 | 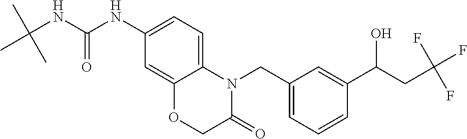 [SMILES: CC(C)(C)NC(=O)Nc1ccc2c(c1)OCC(=O)N2Cc1cccc(C(O)CC(F)(F)F)c1] | 1-(tert-butyl)-3-(3-oxo-4-(3-(3,3,3-trifluoro-1- hydroxypropyl)benzyl)-3,4-dihydro-2H- benzo[b][1,4]oxazin-7-yl)urea |
| | C₂₃H₂₆F₃N₃O₄ | |
| 81 | 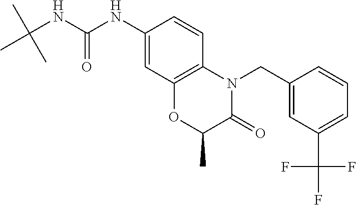 [SMILES: C[C@H]1Oc2cc(NC(=O)NC(C)(C)C)ccc2N(Cc2cccc(C(F)(F)F)c2)C1=O] | (R)-1-(tert-butyl)-3-(2-methyl-3-oxo-4-(3- (trifluoromethyl)benzyl)-3,4-dihydro-2H- benzo[b][1,4]oxazin-7-yl)urea |
| | C₂₂H₂₄F₃N₃O₃ | |
| 82 | 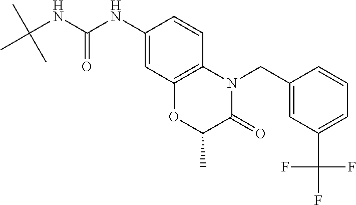 [SMILES: C[C@@H]1Oc2cc(NC(=O)NC(C)(C)C)ccc2N(Cc2cccc(C(F)(F)F)c2)C1=O] | (S)-1-(tert-butyl)-3-(2-methyl-3-oxo-4-(3- (trifluoromethyl)benzyl)-3,4-dihydro-2H- benzo[b][1,4]oxazin-7-yl)urea |
| | C₂₂H₂₄F₃N₃O₃ | |
| 83 | 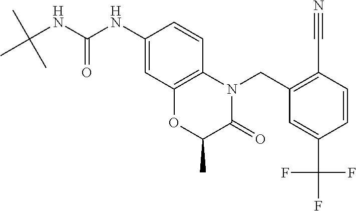 [SMILES: C[C@H]1Oc2cc(NC(=O)NC(C)(C)C)ccc2N(Cc2cc(C(F)(F)F)ccc2C#N)C1=O] | (R)-1-(tert-butyl)-3-(4-(2-cyano-5- (trifluoromethyl)benzyl)-2-methyl-3-oxo-3,4-dihydro-2H- benzo[b][1,4]oxazin-7-yl)urea |
| | C₂₃H₂₃F₃N₄O₃ | |
| 84 | 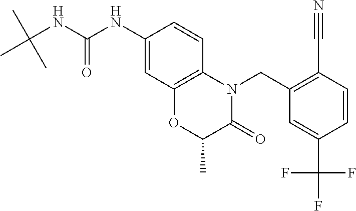 [SMILES: C[C@@H]1Oc2cc(NC(=O)NC(C)(C)C)ccc2N(Cc2cc(C(F)(F)F)ccc2C#N)C1=O] | (S)-1-(tert-butyl)-3-(4-(2-cyano-5- (trifluoromethyl)benzyl)-2-methyl-3-oxo-3,4-dihydro-2H- benzo[b][1,4]oxazin-7-yl)urea |
| | C₂₃H₂₃F₃N₄O₃ | |
| 85 | 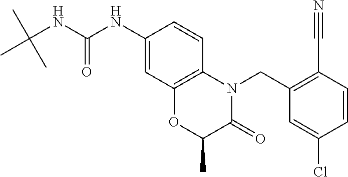 [SMILES: C[C@H]1Oc2cc(NC(=O)NC(C)(C)C)ccc2N(Cc2cc(Cl)ccc2C#N)C1=O] | (R)-1-(tert-butyl)-3-(4-(5-chloro-2-cyanobenzyl)-2- methyl-3-oxo-3,4-dihydro-2H-benzo[b][1,4]oxazin-7- yl)urea |
| | C₂₂H₂₃ClN₄O₃ | |
| 86 | 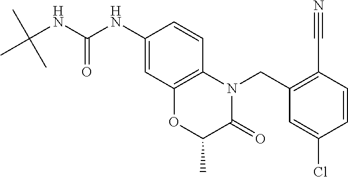 [SMILES: C[C@@H]1Oc2cc(NC(=O)NC(C)(C)C)ccc2N(Cc2cc(Cl)ccc2C#N)C1=O] | (S)-1-(tert-butyl)-3-(4-(5-chloro-2-cyanobenzyl)-2- methyl-3-oxo-3,4-dihydro-2H-benzo[b][1,4]oxazin-7- yl)urea |
| | C₂₂H₂₃ClN₄O₃ | |
| 87 | 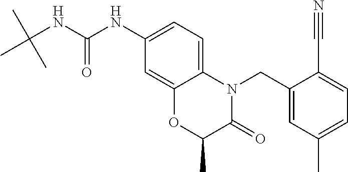 [SMILES: Cc1ccc(C#N)c(CN2C(=O)[C@@H](C)Oc3cc(NC(=O)NC(C)(C)C)ccc32)c1] | (R)-1-(tert-butyl)-3-(4-(2-cyano-5-methylbenzyl)-2- methyl-3-oxo-3,4-dihydro-2H-benzo[b][1,4]oxazin-7- yl)urea |
| | C₂₃H₂₆N₄O₃ | |
| 88 | 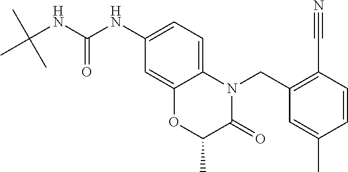 [SMILES: Cc1ccc(C#N)c(CN2C(=O)[C@H](C)Oc3cc(NC(=O)NC(C)(C)C)ccc32)c1] | (S)-1-(tert-butyl)-3-(4-(2-cyano-5-methylbenzyl)-2- methyl-3-oxo-3,4-dihydro-2H-benzo[b][1,4]oxazin-7- yl)urea |
| | C₂₃H₂₆N₄O₃ | |
| 89 | 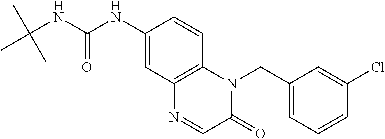 [SMILES: CC(C)(C)NC(=O)Nc1ccc2c(c1)ncc(=O)n2Cc1cccc(Cl)c1] | 1-(tert-butyl)-3-(1-(3-chlorobenzyl)-2-oxo-1,2- dihydroquinoxalin-6-yl)urea |
| | C₂₀H₂₁ClN₄O₂ | |
| 90 | 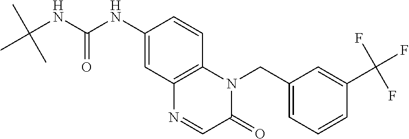 [SMILES: CC(C)(C)NC(=O)Nc1ccc2c(c1)ncc(=O)n2Cc1cccc(C(F)(F)F)c1] | 1-(tert-butyl)-3-(2-oxo-1-(3-(trifluoromethyl)benzyl)-1,2- dihydroquinoxalin-6-yl)urea |
| | C₂₁H₂₁F₃N₄O₂ | |
| 91 | 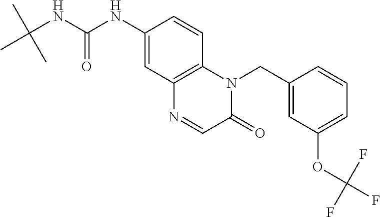 [SMILES: CC(C)(C)NC(=O)Nc1ccc2c(c1)ncc(=O)n2Cc1cccc(OC(F)(F)F)c1] | 1-(tert-butyl)-3-(2-oxo-1-(3-(trifluoromethoxy)benzyl)- 1,2-dihydroquinoxalin-6-yl)urea |
| | C₂₁H₂₁F₃N₄O₃ | |
| 92 | 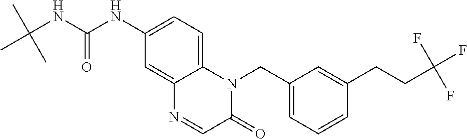 [SMILES: CC(C)(C)NC(=O)Nc1ccc2c(c1)ncc(=O)n2Cc1cccc(CCC(F)(F)F)c1] | 1-(tert-butyl)-3-(2-oxo-1-(3-(3,3,3- trifluoropropyl)benzyl)-1,2-dihydroquinoxalin-6-yl)urea |
| | C₂₃H₂₅F₃N₄O₂ | |
| 93 | 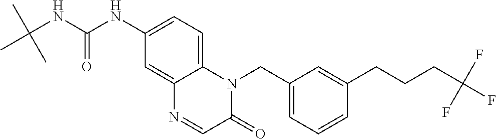 [SMILES: CC(C)(C)NC(=O)Nc1ccc2c(c1)ncc(=O)n2Cc1cccc(CCCC(F)(F)F)c1] | 1-(tert-butyl)-3-(2-oxo-1-(3-(4,4,4-trifluorobutyl)benzyl)- 1,2-dihydroquinoxalin-6-yl)urea |
| | C₂₄H₂₇F₃N₄O₂ | |
| 94 | 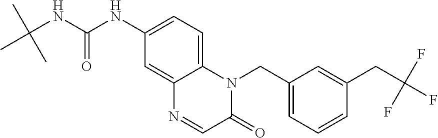 [SMILES: CC(C)(C)NC(=O)Nc1ccc2c(c1)ncc(=O)n2Cc1cccc(CC(F)(F)F)c1] | 1-(tert-butyl)-3-(2-oxo-1-(3-(2,2,2-trifluoroethyl)benzyl)- 1,2-dihydroquinoxalin-6-yl)urea |
| | C₂₂H₂₃F₃N₄O₂ | |
| 95 | 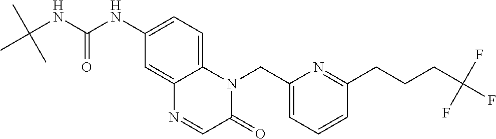 [SMILES: CC(C)(C)NC(=O)Nc1ccc2c(c1)ncc(=O)n2Cc1cccc(CCCC(F)(F)F)n1] | 1-(tert-butyl)-3-(2-oxo-1-((6-(4,4,4-trifluorobutyl)pyridin- 2-yl)methyl)-1,2-dihydroquinoxalin-6-yl)urea |
| | C₂₃H₂₆F₃N₅O₂ | |
| 96 | 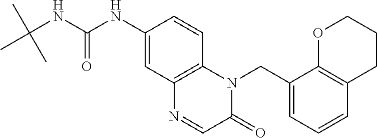 [SMILES: CC(C)(C)NC(=O)Nc1ccc2c(c1)ncc(=O)n2Cc1cccc2c1OCCC2] | 1-(tert-butyl)-3-(1-(chroman-8-ylmethyl)-2-oxo-1,2- dihydroquinoxalin-6-yl)urea |
| | C₂₃H₂₆N₄O₃ | |
| 97 | 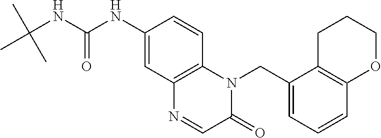 [SMILES: CC(C)(C)NC(=O)Nc1ccc2c(c1)ncc(=O)n2Cc1cccc2c1CCCO2] | 1-(tert-butyl)-3-(1-(chroman-5-ylmethyl)-2-oxo-1,2- dihydroquinoxalin-6-yl)urea |
| | C₂₃H₂₆N₄O₃ | |
| 98 | 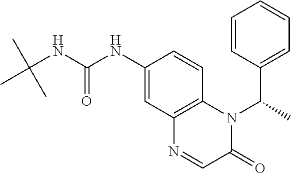 [SMILES: C[C@@H](c1ccccc1)n1c(=O)cnc2cc(NC(=O)NC(C)(C)C)ccc21] | (S)-1-(tert-butyl)-3-(2-oxo-1-(1-phenylethyl)-1,2- dihydroquinoxalin-6-yl)urea |
| | C₂₁H₂₄N₄O₂ | |
| 99 | 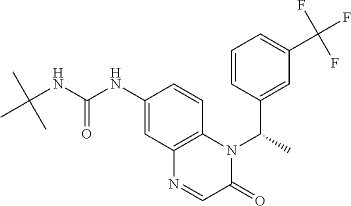 [SMILES: C[C@@H](c1cccc(C(F)(F)F)c1)n1c(=O)cnc2cc(NC(=O)NC(C)(C)C)ccc21] | (S)-1-(tert-butyl)-3-(2-oxo-1-(1-(3- (trifluoromethyl)phenyl)ethyl)-1,2-dihydroquinoxalin-6- yl)urea |
| | C₂₂H₂₃F₃N₄O₂ | |
| 100 | 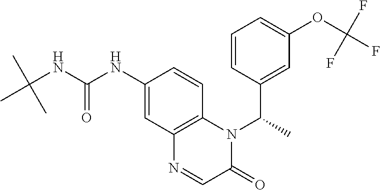 [SMILES: C[C@@H](c1cccc(OC(F)(F)F)c1)n1c(=O)cnc2cc(NC(=O)NC(C)(C)C)ccc21] | (S)-1-(tert-butyl)-3-(2-oxo-1-(1-(3- (trifluoromethoxy)phenyl)ethyl)-1,2-dihydroquinoxalin-6- yl)urea |
| | C₂₂H₂₃F₃N₄O₃ | |
| 101 | 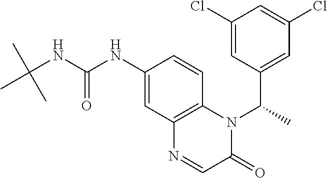 [SMILES: C[C@@H](c1cc(Cl)cc(Cl)c1)n1c(=O)cnc2cc(NC(=O)NC(C)(C)C)ccc21] | (S)-1-(tert-butyl)-3-(1-(1-(3,5-dichlorophenyl)ethyl)-2- oxo-1,2-dihydroquinoxalin-6-yl)urea |
| | C₂₁H₂₂Cl₂N₄O₂ | |
| 102 | 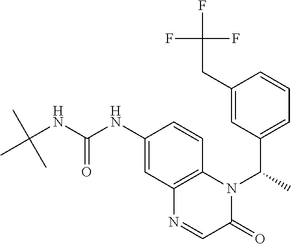 [SMILES: C[C@@H](c1cccc(CC(F)(F)F)c1)n1c(=O)cnc2cc(NC(=O)NC(C)(C)C)ccc21] | (S)-1-(tert-butyl)-3-(2-oxo-1-(1-(3-(2,2,2- trifluoroethyl)phenyl)ethyl)-1,2-dihydroquinoxalin-6- yl)urea |
| | C₂₃H₂₅F₃N₄O₂ | |
| 103 | 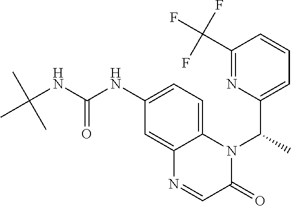 [SMILES: C[C@@H](c1cccc(C(F)(F)F)n1)n1c(=O)cnc2cc(NC(=O)NC(C)(C)C)ccc21] | (S)-1-(tert-butyl)-3-(2-oxo-1-(1-(6- (trifluoromethyl)pyridin-2-yl)ethyl)-1,2- dihydroquinoxalin-6-yl)urea |
| | C₂₁H₂₂F₃N₅O₂ | |
| 104 | 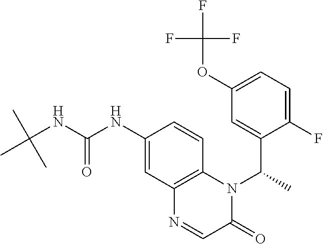 [SMILES: C[C@@H](c1cc(OC(F)(F)F)ccc1F)n1c(=O)cnc2cc(NC(=O)NC(C)(C)C)ccc21] | (S)-1-(tert-butyl)-3-(1-(1-(2-fluoro-5- (trifluoromethoxy)phenyl)ethyl)-2-oxo-1,2- dihydroquinoxalin-6-yl)urea |
| | C₂₂H₂₂F₄N₄O₃ | |
| 105 | 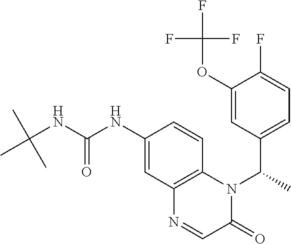 [SMILES: C[C@@H](c1ccc(F)c(OC(F)(F)F)c1)n1c(=O)cnc2cc(NC(=O)NC(C)(C)C)ccc21] | (S)-1-(tert-butyl)-3-(1-(1-(4-fluoro-3- (trifluoromethoxy)phenyl)ethyl)-2-oxo-1,2- dihydroquinoxalin-6-yl)urea |
| | C₂₂H₂₂F₄N₄O₃ | |
| 106 | 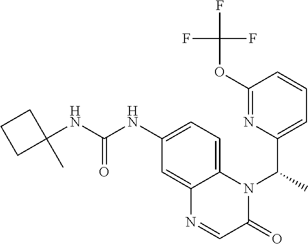 [SMILES: C[C@@H](c1cccc(OC(F)(F)F)n1)n1c(=O)cnc2cc(NC(=O)NC3(C)CCC3)ccc21] | (S)-1-(1-methylcyclobutyl)-3-(2-oxo-1-(1-(6- (trifluoromethoxy)pyridin-2-yl)ethyl)-1,2- dihydroquinoxalin-6-yl)urea |
| | C₂₂H₂₂F₃N₅O₃ | |
| 107 | 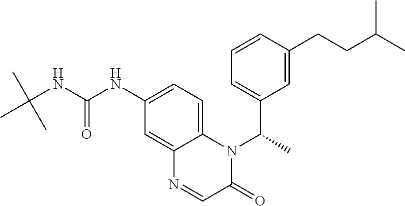 [SMILES: CC(C)CCc1cccc([C@H](C)n2c(=O)cnc3cc(NC(=O)NC(C)(C)C)ccc32)c1] | (S)-1-(tert-butyl)-3-(1-(1-(3-isopentylphenyl)ethyl)-2- oxo-1,2-dihydroquinoxalin-6-yl)urea |
| | C₂₆H₃₄N₄O₂ | |
| 108 | 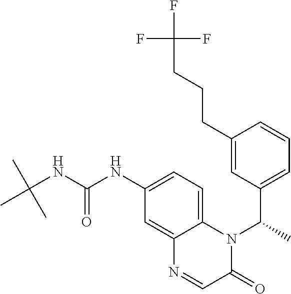 [SMILES: C[C@@H](c1cccc(CCCC(F)(F)F)c1)n1c(=O)cnc2cc(NC(=O)NC(C)(C)C)ccc21] | (S)-1-(tert-butyl)-3-(2-oxo-1-(1-(3-(4,4,4- trifluorobutyl)phenyl)ethyl)-1,2-dihydroquinoxalin-6- yl)urea |
| | C₂₅H₂₉F₃N₄O₂ | |
| 109 | 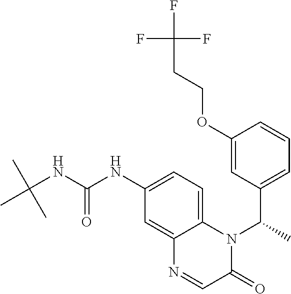 [SMILES: C[C@@H](c1cccc(OCCC(F)(F)F)c1)n1c(=O)cnc2cc(NC(=O)NC(C)(C)C)ccc21] | (S)-1-(tert-butyl)-3-(2-oxo-1-(1-(3-(3,3,3- trifluoropropoxy)phenyl)ethyl)-1,2-dihydroquinoxalin-6- yl)urea |
| | C₂₄H₂₇F₃N₄O₃ | |
| 110 | 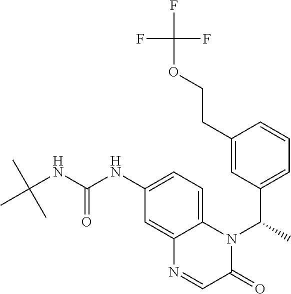 [SMILES: C[C@@H](c1cccc(CCOC(F)(F)F)c1)n1c(=O)cnc2cc(NC(=O)NC(C)(C)C)ccc21] | (S)-1-(tert-butyl)-3-(2-oxo-1-(1-(3-(2- (trifluoromethoxy)ethyl)phenyl)ethyl)-1,2- dihydroquinoxalin-6-yl)urea |
| | C₂₄H₂₇F₃N₄O₃ | |
| 111 | 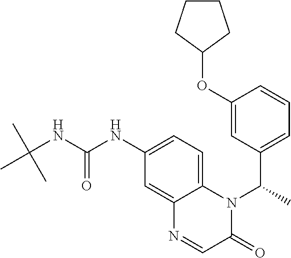 [SMILES: C[C@@H](c1cccc(OC2CCCC2)c1)n1c(=O)cnc2cc(NC(=O)NC(C)(C)C)ccc21] | (S)-1-(tert-butyl)-3-(1-(1-(3- (cyclopentyloxy)phenyl)ethyl)-2-oxo-1,2- dihydroquinoxalin-6-yl)urea |
| | C₂₆H₃₂N₄O₃ | |
| 112 | 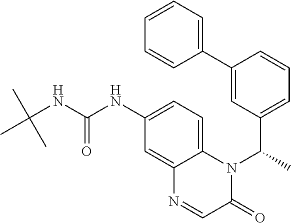 [SMILES: C[C@@H](c1cccc(-c2ccccc2)c1)n1c(=O)cnc2cc(NC(=O)NC(C)(C)C)ccc21] | (S)-1-(1-(1-([1,1′-biphenyl]-3-yl)ethyl)-2-oxo-1,2- dihydroquinoxalin-6-yl)-3-(tert-butyl)urea |
| | C₂₇H₂₈N₄O₂ | |
| 113 | 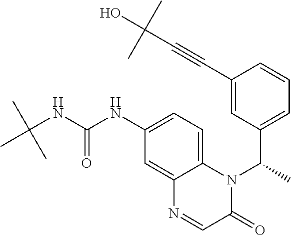 [SMILES: C[C@@H](c1cccc(C#CC(C)(C)O)c1)n1c(=O)cnc2cc(NC(=O)NC(C)(C)C)ccc21] | (S)-1-(tert-butyl)-3-(1-(1-(3-(3-hydroxy-3-methylbut-1- yn-1-yl)phenyl)ethyl)-2-oxo-1,2-dihydroquinoxalin-6- yl)urea |
| | C₂₆H₃₀N₄O₃ | |
| 114 | 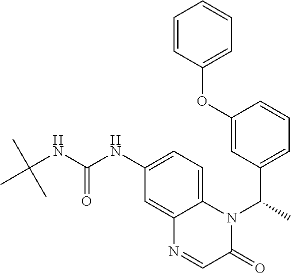 [SMILES: C[C@@H](c1cccc(Oc2ccccc2)c1)n1c(=O)cnc2cc(NC(=O)NC(C)(C)C)ccc21] | (S)-1-(tert-butyl)-3-(2-oxo-1-(1-(3-phenoxyphenyl)ethyl)- 1,2-dihydroquinoxalin-6-yl)urea |
| | C₂₇H₂₈N₄O₃ | |
| 115 | 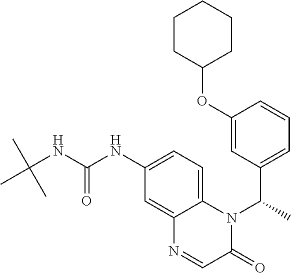 [SMILES: C[C@@H](c1cccc(OC2CCCCC2)c1)n1c(=O)cnc2cc(NC(=O)NC(C)(C)C)ccc21] | (S)-1-(tert-butyl)-3-(1-(1-(3- (cyclohexyloxy)phenyl)ethyl)-2-oxo-1,2- dihydroquinoxalin-6-yl)urea |
| | C₂₇H₃₄N₄O₃ | |
| 116 | 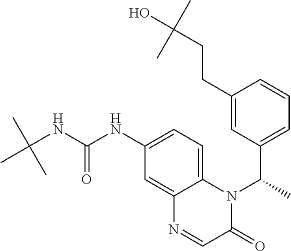 [SMILES: C[C@@H](c1cccc(CCC(C)(C)O)c1)n1c(=O)cnc2cc(NC(=O)NC(C)(C)C)ccc21] | (S)-1-(tert-butyl)-3-(1-(1-(3-(3-hydroxy-3- methylbutyl)phenyl)ethyl)-2-oxo-1,2-dihydroquinoxalin- 6-yl)urea |
| | C₂₆H₃₄N₄O₃ | |
| 117 | 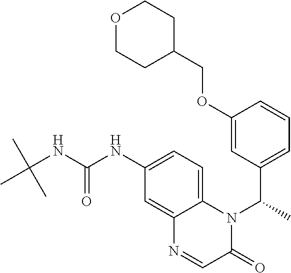 [SMILES: C[C@@H](c1cccc(OCC2CCOCC2)c1)n1c(=O)cnc2cc(NC(=O)NC(C)(C)C)ccc21] | (S)-1-(tert-butyl)-3-(2-oxo-1-(1-(3-((tetrahydro-2H-pyran- 4-yl)methoxy)phenyl)ethyl)-1,2-dihydroquinoxalin-6- yl)urea |
| | C₂₇H₃₄N₄O₄ | |
| 118 | 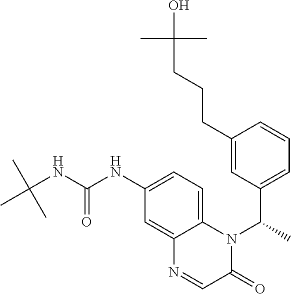 [SMILES: C[C@@H](c1cccc(CCCC(C)(C)O)c1)n1c(=O)cnc2cc(NC(=O)NC(C)(C)C)ccc21] | (S)-1-(tert-butyl)-3-(1-(1-(3-(4-hydroxy-4- methylpentyl)phenyl)ethyl)-2-oxo-1,2-dihydroquinoxalin- 6-yl)urea |
| | C₂₇H₃₆N₄O₃ | |
| 119 | 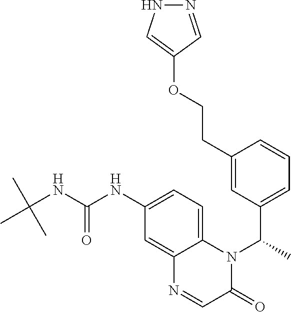 [SMILES: C[C@@H](c1cccc(CCOc2cn[nH]c2)c1)n1c(=O)cnc2cc(NC(=O)NC(C)(C)C)ccc21] | (S)-1-(1-(1-(3-(2-((1H-pyrazol-4- yl)oxy)ethyl)phenyl)ethyl)-2-oxo-1,2-dihydroquinoxalin- 6-yl)-3-(tert-butyl)urea |
| | C₂₆H₃₀N₆O₃ | |
| 120 | 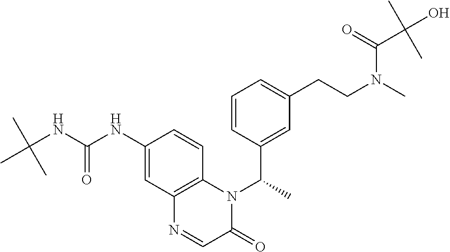 [SMILES: C[C@@H](c1cccc(CCN(C)C(=O)C(C)(C)O)c1)n1c(=O)cnc2cc(NC(=O)NC(C)(C)C)ccc21] | (S)-N-(3-(1-(6-(3-(tert-butyl)ureido)-2-oxoquinoxalin- 1(2H)-yl)ethyl)phenethyl)-2-hydroxy-N,2- dimethylpropanamide |
| | C₂₈H₃₇N₅O₄ | |
| 121 | 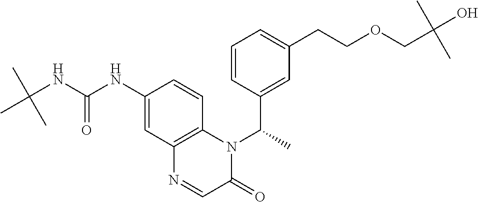 [SMILES: C[C@@H](c1cccc(CCOCC(C)(C)O)c1)n1c(=O)cnc2cc(NC(=O)NC(C)(C)C)ccc21] | (S)-1-(tert-butyl)-3-(1-(1-(3-(2-(2-hydroxy-2- methylpropoxy)ethyl)phenyl)ethyl)-2-oxo-1,2- dihydroquinoxalin-6-yl)urea |
| | C₂₇H₃₆N₄O₄ | |
| 122 | 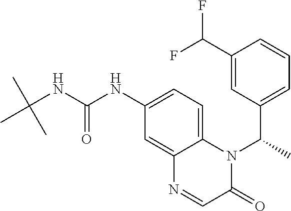 [SMILES: C[C@@H](c1cccc(C(F)F)c1)n1c(=O)cnc2cc(NC(=O)NC(C)(C)C)ccc21] | (S)-1-(tert-butyl)-3-(1-(1-(3- (difluoromethyl)phenyl)ethyl)-2-oxo-1,2- dihydroquinoxalin-6-yl)urea |
| | C₂₂H₂₄F₂N₄O₂ | |
| 123 | 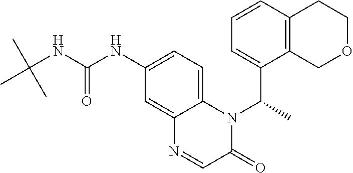 [SMILES: C[C@@H](c1cccc2c1COCC2)n1c(=O)cnc2cc(NC(=O)NC(C)(C)C)ccc21] | (S)-1-(tert-butyl)-3-(1-(1-(isochroman-8-yl)ethyl)-2-oxo- 1,2-dihydroquinoxalin-6-yl)urea |
| | C₂₄H₂₈N₄O₃ | |
| 124 | 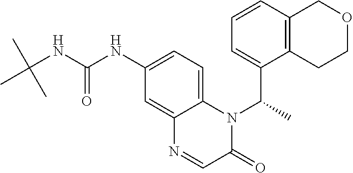 [SMILES: C[C@@H](c1cccc2c1CCOC2)n1c(=O)cnc2cc(NC(=O)NC(C)(C)C)ccc21] | (S)-1-(tert-butyl)-3-(1-(1-(isochroman-5-yl)ethyl)-2-oxo- 1,2-dihydroquinoxalin-6-yl)urea |
| | C₂₄H₂₈N₄O₃ | |
| 125 | 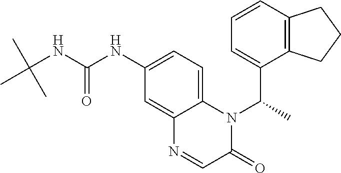 [SMILES: C[C@@H](c1cccc2c1CCC2)n1c(=O)cnc2cc(NC(=O)NC(C)(C)C)ccc21] | (S)-1-(tert-butyl)-3-(1-(1-(2,3-dihydro-1H-inden-4- yl)ethyl)-2-oxo-1,2-dihydroquinoxalin-6-yl)urea |
| | C₂₄H₂₈N₄O₂ | |
| 126 | 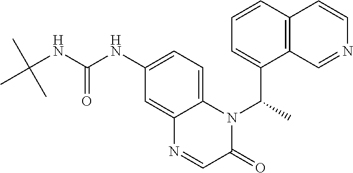 [SMILES: C[C@@H](c1cccc2ccncc12)n1c(=O)cnc2cc(NC(=O)NC(C)(C)C)ccc21] | (S)-1-(tert-butyl)-3-(1-(1-(isoquinolin-8-yl)ethyl)-2-oxo- 1,2-dihydroquinoxalin-6-yl)urea |
| | C₂₄H₂₅N₅O₂ | |
| 127 | 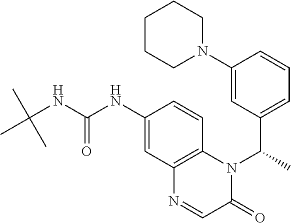 [SMILES: C[C@@H](c1cccc(N2CCCCC2)c1)n1c(=O)cnc2cc(NC(=O)NC(C)(C)C)ccc21] | (S)-1-(tert-butyl)-3-(2-oxo-1-(1-(3-(piperidin-1- yl)phenyl)ethyl)-1,2-dihydroquinoxalin-6-yl)urea |
| | C₂₆H₃₃N₅O₂ | |
| 128 | 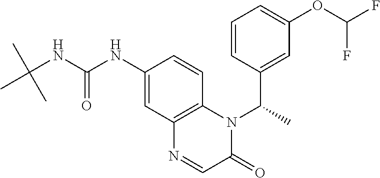 [SMILES: C[C@@H](c1cccc(OC(F)F)c1)n1c(=O)cnc2cc(NC(=O)NC(C)(C)C)ccc21] | (S)-1-(tert-butyl)-3-(1-(1-(3- (difluoromethoxy)phenyl)ethyl)-2-oxo-1,2- dihydroquinoxalin-6-yl)urea |
| | C₂₂H₂₄F₂N₄O₃ | |
| 129 | 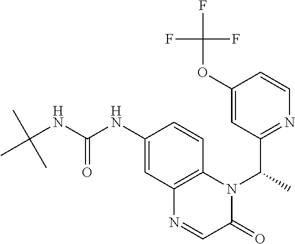 [SMILES: C[C@@H](c1cc(OC(F)(F)F)ccn1)n1c(=O)cnc2cc(NC(=O)NC(C)(C)C)ccc21] | (S)-1-(tert-butyl)-3-(2-oxo-1-(1-(4- (trifluoromethoxy)pyridin-2-yl)ethyl)-1,2- dihydroquinoxalin-6-yl)urea |
| | C₂₁H₂₂F₃N₅O₃ | |
| 130 | 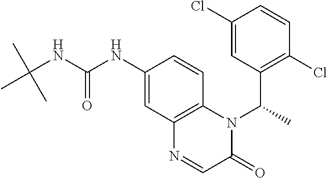 [SMILES: C[C@@H](c1cc(Cl)ccc1Cl)n1c(=O)cnc2cc(NC(=O)NC(C)(C)C)ccc21] | (S)-1-(tert-butyl)-3-(1-(1-(2,5-dichlorophenyl)ethyl)-2- oxo-1,2-dihydroquinoxalin-6-yl)urea |
| | C₂₁H₂₂Cl₂N₄O₂ | |
| 131 | 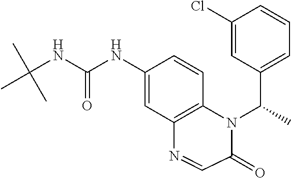 [SMILES: C[C@@H](c1cccc(Cl)c1)n1c(=O)cnc2cc(NC(=O)NC(C)(C)C)ccc21] | (S)-1-(tert-butyl)-3-(1-(1-(3-chlorophenyl)ethyl)-2-oxo- 1,2-dihydroquinoxalin-6-yl)urea |
| | C₂₁H₂₃ClN₄O₂ | |
| 132 | 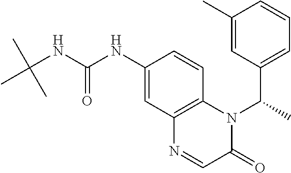 [SMILES: Cc1cccc([C@H](C)n2c(=O)cnc3cc(NC(=O)NC(C)(C)C)ccc32)c1] | (S)-1-(tert-butyl)-3-(2-oxo-1-(1-(m-tolyl)ethyl)-1,2- dihydroquinoxalin-6-yl)urea |
| | C₂₂H₂₆N₄O₂ | |
| 133 | 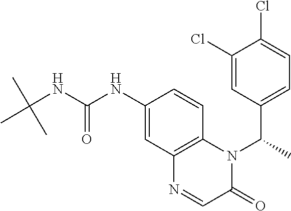 [SMILES: C[C@@H](c1ccc(Cl)c(Cl)c1)n1c(=O)cnc2cc(NC(=O)NC(C)(C)C)ccc21] | (S)-1-(tert-butyl)-3-(1-(1-(3,4-dichlorophenyl)ethyl)-2- oxo-1,2-dihydroquinoxalin-6-yl)urea |
| | C₂₁H₂₂Cl₂N₄O₂ | |
| 134 | 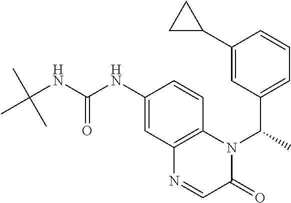 [SMILES: C[C@@H](c1cccc(C2CC2)c1)n1c(=O)cnc2cc(NC(=O)NC(C)(C)C)ccc21] | (S)-1-(tert-butyl)-3-(1-(1-(3-cyclopropylphenyl)ethyl)-2- oxo-1,2-dihydroquinoxalin-6-yl)urea |
| | C₂₄H₂₈N₄O₂ | |
| 135 | 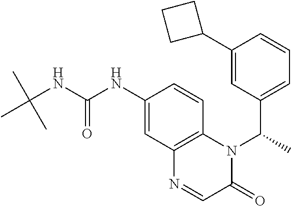 [SMILES: C[C@@H](c1cccc(C2CCC2)c1)n1c(=O)cnc2cc(NC(=O)NC(C)(C)C)ccc21] | (S)-1-(tert-butyl)-3-(1-(1-(3-cyclobutylphenyl)ethyl)-2- oxo-1,2-dihydroquinoxalin-6-yl)urea |
| | C₂₅H₃₀N₄O₂ | |
| 136 | 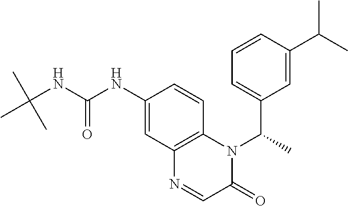 [SMILES: CC(C)c1cccc([C@H](C)n2c(=O)cnc3cc(NC(=O)NC(C)(C)C)ccc32)c1] | (S)-1-(tert-butyl)-3-(1-(1-(3-isopropylphenyl)ethyl)-2- oxo-1,2-dihydroquinoxalin-6-yl)urea |
| | C₂₄H₃₀N₄O₂ | |
| 137 | 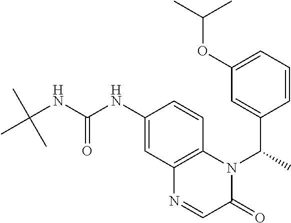 [SMILES: CC(C)Oc1cccc([C@H](C)n2c(=O)cnc3cc(NC(=O)NC(C)(C)C)ccc32)c1] | (S)-1-(tert-butyl)-3-(1-(1-(3-isopropoxyphenyl)ethyl)-2- oxo-1,2-dihydroquinoxalin-6-yl)urea |
| | C₂₄H₃₀N₄O₃ | |
| 138 | 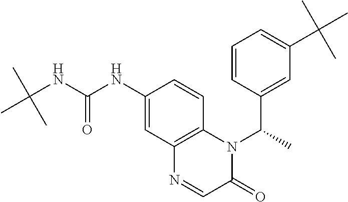 [SMILES: C[C@@H](c1cccc(C(C)(C)C)c1)n1c(=O)cnc2cc(NC(=O)NC(C)(C)C)ccc21] | (S)-1-(tert-butyl)-3-(1-(1-(3-(tert-butyl)phenyl)ethyl)-2- oxo-1,2-dihydroquinoxalin-6-yl)urea |
| | C₂₅H₃₂N₄O₂ | |
| 139 | 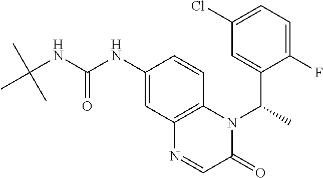 [SMILES: C[C@@H](c1cc(Cl)ccc1F)n1c(=O)cnc2cc(NC(=O)NC(C)(C)C)ccc21] | (S)-1-(tert-butyl)-3-(1-(1-(5-chloro-2-fluorophenyl)ethyl)- 2-oxo-1,2-dihydroquinoxalin-6-yl)urea |
| | C₂₁H₂₂ClFN₄O₂ | |
| 140 | 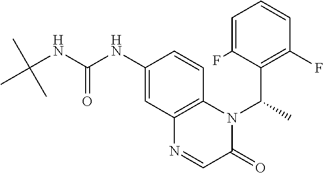 [SMILES: C[C@@H](c1c(F)cccc1F)n1c(=O)cnc2cc(NC(=O)NC(C)(C)C)ccc21] | (S)-1-(tert-butyl)-3-(1-(1-(2,6-difluorophenyl)ethyl)-2- oxo-1,2-dihydroquinoxalin-6-yl)urea |
| | C₂₁H₂₂F₂N₄O₂ | |
| 141 | 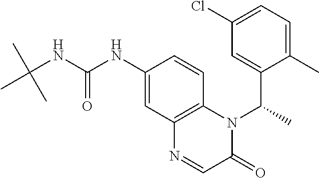 [SMILES: Cc1ccc(Cl)cc1[C@H](C)n1c(=O)cnc2cc(NC(=O)NC(C)(C)C)ccc21] | (S)-1-(tert-butyl)-3-(1-(1-(5-chloro-2- methylphenyl)ethyl)-2-oxo-1,2-dihydroquinoxalin-6- yl)urea |
| | C₂₂H₂₅ClN₄O₂ | |
| 142 | 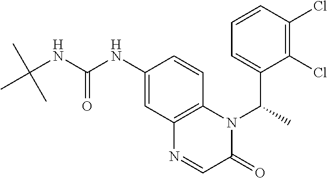 [SMILES: C[C@@H](c1cccc(Cl)c1Cl)n1c(=O)cnc2cc(NC(=O)NC(C)(C)C)ccc21] | (S)-1-(tert-butyl)-3-(1-(1-(2,3-dichlorophenyl)ethyl)-2- oxo-1,2-dihydroquinoxalin-6-yl)urea |
| | C₂₁H₂₂Cl₂N₄O₂ | |
| 143 | 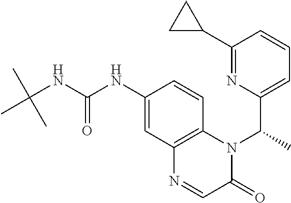 [SMILES: C[C@@H](c1cccc(C2CC2)n1)n1c(=O)cnc2cc(NC(=O)NC(C)(C)C)ccc21] | (S)-1-(tert-butyl)-3-(1-(1-(6-cyclopropylpyridin-2- yl)ethyl)-2-oxo-1,2-dihydroquinoxalin-6-yl)urea |
| | C₂₃H₂₇N₅O₂ | |
| 144 | 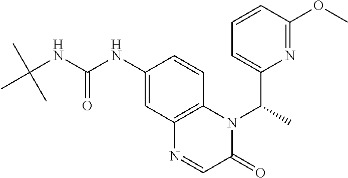 [SMILES: COc1cccc([C@H](C)n2c(=O)cnc3cc(NC(=O)NC(C)(C)C)ccc32)n1] | (S)-1-(tert-butyl)-3-(1-(1-(6-methoxypyridin-2-yl)ethyl)- 2-oxo-1,2-dihydroquinoxalin-6-yl)urea |
| | C₂₁H₂₅N₅O₃ | |
| 145 | 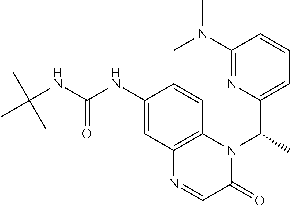 [SMILES: C[C@@H](c1cccc(N(C)C)n1)n1c(=O)cnc2cc(NC(=O)NC(C)(C)C)ccc21] | (S)-1-(tert-butyl)-3-(1-(1-(6-(dimethylamino)pyridin-2- yl)ethyl)-2-oxo-1,2-dihydroquinoxalin-6-yl)urea |
| | C₂₂H₂₈N₆O₂ | |
| 146 | 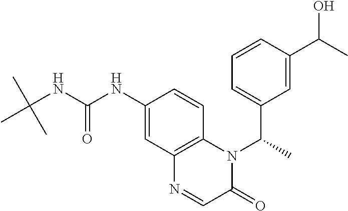 [SMILES: CC(O)c1cccc([C@H](C)n2c(=O)cnc3cc(NC(=O)NC(C)(C)C)ccc32)c1] | 1-(tert-butyl)-3-(1-((1S)-1-(3-(1- hydroxyethyl)phenyl)ethyl)-2-oxo-1,2-dihydroquinoxalin- 6-yl)urea |
| | C₂₃H₂₈N₄O₃ | |
| 147 | 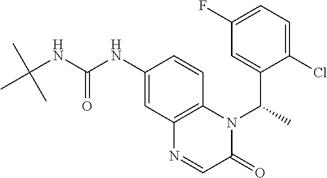 [SMILES: C[C@@H](c1cc(F)ccc1Cl)n1c(=O)cnc2cc(NC(=O)NC(C)(C)C)ccc21] | (S)-1-(tert-butyl)-3-(1-(1-(2-chloro-5-fluorophenyl)ethyl)- 2-oxo-1,2-dihydroquinoxalin-6-yl)urea |
| | C₂₁H₂₂ClFN₄O₂ | |
| 148 | 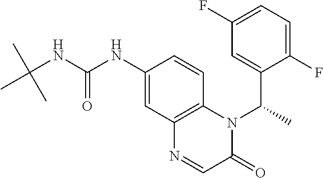 [SMILES: C[C@@H](c1cc(F)ccc1F)n1c(=O)cnc2cc(NC(=O)NC(C)(C)C)ccc21] | (S)-1-(tert-butyl)-3-(1-(1-(2,5-difluorophenyl)ethyl)-2- oxo-1,2-dihydroquinoxalin-6-yl)urea |
| | C₂₁H₂₂F₂N₄O₂ | |
| 149 | 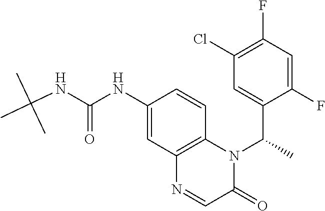 [SMILES: C[C@@H](c1cc(Cl)c(F)cc1F)n1c(=O)cnc2cc(NC(=O)NC(C)(C)C)ccc21] | (S)-1-(tert-butyl)-3-(1-(1-(5-chloro-2,4- difluorophenyl)ethyl)-2-oxo-1,2-dihydroquinoxalin-6- yl)urea |
| | C₂₁H₂₁ClF₂N₄O₂ | |
| 150 | 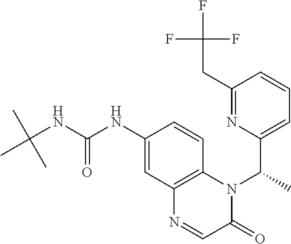 [SMILES: C[C@@H](c1cccc(CC(F)(F)F)n1)n1c(=O)cnc2cc(NC(=O)NC(C)(C)C)ccc21] | (S)-1-(tert-butyl)-3-(2-oxo-1-(1-(6-(2,2,2- trifluoroethyl)pyridin-2-yl)ethyl)-1,2-dihydroquinoxalin- 6-yl)urea |
| | C₂₂H₂₄F₃N₅O₂ | |
| 151 | 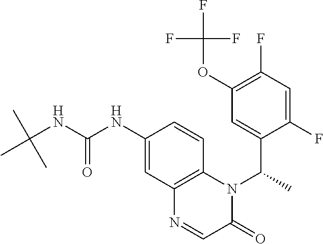 [SMILES: C[C@@H](c1cc(OC(F)(F)F)c(F)cc1F)n1c(=O)cnc2cc(NC(=O)NC(C)(C)C)ccc21] | (S)-1-(tert-butyl)-3-(1-(1-(2,4-difluoro-5- (trifluoromethoxy)phenyl)ethyl)-2-oxo-1,2- dihydroquinoxalin-6-yl)urea |
| | C₂₂H₂₁F₅N₄O₃ | |
| 152 | 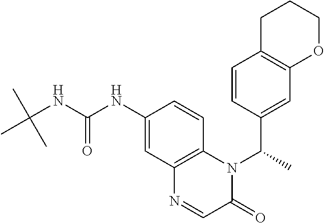 [SMILES: C[C@@H](c1ccc2c(c1)OCCC2)n1c(=O)cnc2cc(NC(=O)NC(C)(C)C)ccc21] | (S)-1-(tert-butyl)-3-(1-(1-(chroman-7-yl)ethyl)-2-oxo-1,2- dihydroquinoxalin-6-yl)urea |
| | C₂₄H₂₈N₄O₃ | |
| 153 | 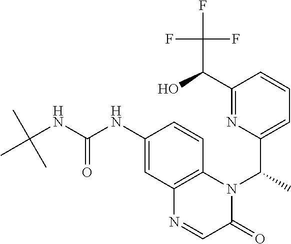 [SMILES: C[C@@H](c1cccc([C@@H](O)C(F)(F)F)n1)n1c(=O)cnc2cc(NC(=O)NC(C)(C)C)ccc21] | 1-(tert-butyl)-3-(2-oxo-1-((S)-1-(6-((R)-2,2,2-trifluoro-1- hydroxyethyl)pyridin-2-yl)ethyl)-1,2-dihydroquinoxalin- 6-yl)urea |
| | C₂₂H₂₄F₃N₅O₃ | |
| 154 | 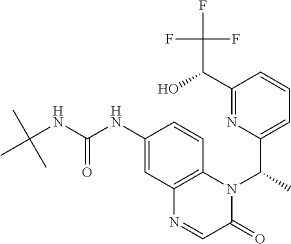 [SMILES: C[C@@H](c1cccc([C@H](O)C(F)(F)F)n1)n1c(=O)cnc2cc(NC(=O)NC(C)(C)C)ccc21] | 1-(tert-butyl)-3-(2-oxo-1-((S)-1-(6-((S)-2,2,2-trifluoro-1- hydroxyethyl)pyridin-2-yl)ethyl)-1,2-dihydroquinoxalin- 6-yl)urea |
| | C₂₂H₂₄F₃N₅O₃ | |
| 155 | 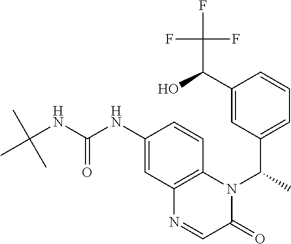 [SMILES: C[C@@H](c1cccc([C@@H](O)C(F)(F)F)c1)n1c(=O)cnc2cc(NC(=O)NC(C)(C)C)ccc21] | 1-(tert-butyl)-3-(2-oxo-1-((S)-1-(3-((R)-2,2,2-trifluoro-1- hydroxyethyl)phenyl)ethyl)-1,2-dihydroquinoxalin-6- yl)urea |
| | C₂₃H₂₅F₃N₄O₃ | |
| 156 | 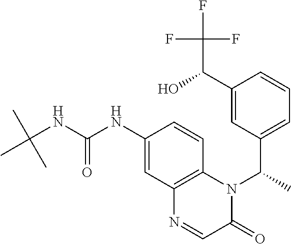 [SMILES: C[C@@H](c1cccc([C@H](O)C(F)(F)F)c1)n1c(=O)cnc2cc(NC(=O)NC(C)(C)C)ccc21] | 1-(tert-butyl)-3-(2-oxo-1-((S)-1-(3-((S)-2,2,2-trifluoro-1- hydroxyethyl)phenyl)ethyl)-1,2-dihydroquinoxalin-6- yl)urea |
| | C₂₃H₂₅F₃N₄O₃ | |
| 157 | 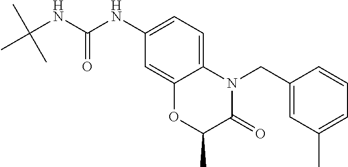 [SMILES: Cc1cccc(CN2C(=O)[C@@H](C)Oc3cc(NC(=O)NC(C)(C)C)ccc32)c1] | (R)-1-(tert-butyl)-3-(2-methyl-4-(3-methylbenzyl)-3-oxo- 3,4-dihydro-2H-benzo[b][1,4]oxazin-7-yl)urea |
| | C₂₂H₂₇N₃O₃ | |
| 158 | 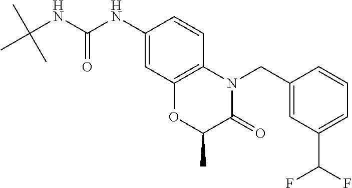 [SMILES: C[C@H]1Oc2cc(NC(=O)NC(C)(C)C)ccc2N(Cc2cccc(C(F)F)c2)C1=O] | (R)-1-(tert-butyl)-3-(4-(3-(difluoromethyl)benzyl)-2- methyl-3-oxo-3,4-dihydro-2H-benzo[b][1,4]oxazin-7- yl)urea |
| | C₂₂H₂₅F₂N₃O₃ | |
| 159 | 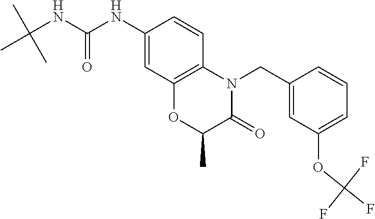 [SMILES: C[C@H]1Oc2cc(NC(=O)NC(C)(C)C)ccc2N(Cc2cccc(OC(F)(F)F)c2)C1=O] | (R)-1-(tert-butyl)-3-(2-methyl-3-oxo-4-(3- (trifluoromethoxy)benzyl)-3,4-dihydro-2H- benzo[b][1,4]oxazin-7-yl)urea |
| | C₂₂H₂₄F₃N₃O₄ | |
| 160 | 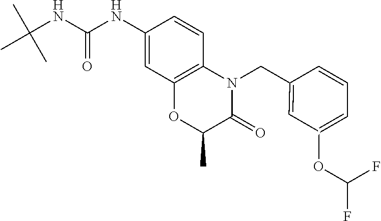 [SMILES: C\C=C(/C=C\C=C\OC(F)F)CN1C(=O)[C@@H](C)Oc2cc(NC(=O)NC(C)(C)C)ccc21] | (R)-1-(tert-butyl)-3-(4-(3-(difluoromethoxy)benzyl)-2- methyl-3-oxo-3,4-dihydro-2H-benzo[b][1,4]oxazin-7- yl)urea |
| | C₂₂H₂₅F₂N₃O₄ | |
| 161 | 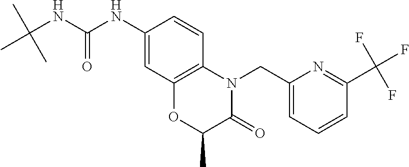 [SMILES: C[C@H]1Oc2cc(NC(=O)NC(C)(C)C)ccc2N(Cc2cccc(C(F)(F)F)n2)C1=O] | (R)-1-(tert-butyl)-3-(2-methyl-3-oxo-4-((6- (trifluoromethyl)pyridin-2-yl)methyl)-3,4-dihydro-2H- benzo[b][1,4]oxazin-7-yl)urea |
| | C₂₁H₂₃F₃N₄O₃ | |
| 162 | 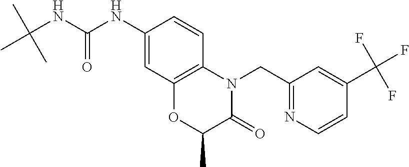 [SMILES: C[C@H]1Oc2cc(NC(=O)NC(C)(C)C)ccc2N(Cc2cc(C(F)(F)F)ccn2)C1=O] | (R)-1-(tert-butyl)-3-(2-methyl-3-oxo-4-((4- (trifluoromethyl)pyridin-2-yl)methyl)-3,4-dihydro-2H- benzo[b][1,4]oxazin-7-yl)urea |
| | C₂₁H₂₃F₃N₄O₃ | |
| 163 | 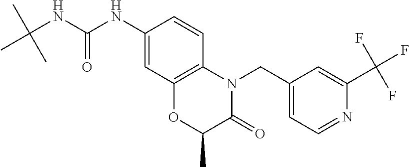 [SMILES: C[C@H]1Oc2cc(NC(=O)NC(C)(C)C)ccc2N(Cc2ccnc(C(F)(F)F)c2)C1=O] | (R)-1-(tert-butyl)-3-(2-methyl-3-oxo-4-((2- (trifluoromethyl)pyridin-4-yl)methyl)-3,4-dihydro-2H- benzo[b][1,4]oxazin-7-yl)urea |
| | C₂₁H₂₃F₃N₄O₃ | |
| 164 | 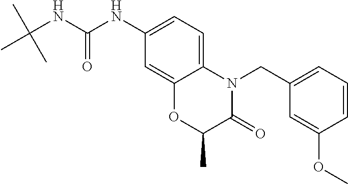 [SMILES: COc1cccc(CN2C(=O)[C@@H](C)Oc3cc(NC(=O)NC(C)(C)C)ccc32)c1] | (R)-1-(tert-butyl)-3-(4-(3-methoxybenzyl)-2-methyl-3- oxo-3,4-dihydro-2H-benzo[b][1,4]oxazin-7-yl)urea |
| | C₂₂H₂₇N₃O₄ | |
| 165 | 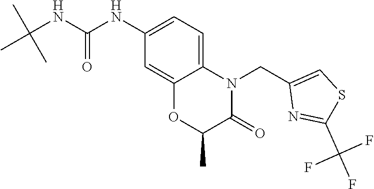 [SMILES: C[C@H]1Oc2cc(NC(=O)NC(C)(C)C)ccc2N(Cc2csc(C(F)(F)F)n2)C1=O] | (R)-1-(tert-butyl)-3-(2-methyl-3-oxo-4-((2- (trifluoromethyl)thiazol-4-yl)methyl)-3,4-dihydro-2H- benzo[b][1,4]oxazin-7-yl)urea |
| | C₁₉H₂₁F₃N₄O₃S | |
| 166 | 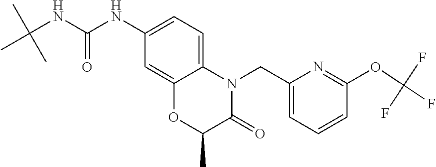 [SMILES: C[C@H]1Oc2cc(NC(=O)NC(C)(C)C)ccc2N(Cc2cccc(OC(F)(F)F)n2)C1=O] | (R)-1-(tert-butyl)-3-(2-methyl-3-oxo-4-((6- (trifluoromethoxy)pyridin-2-yl)methyl)-3,4-dihydro-2H- benzo[b][1,4]oxazin-7-yl)urea |
| | C₂₁H₂₃F₃N₄O₄ | |
| 167 | 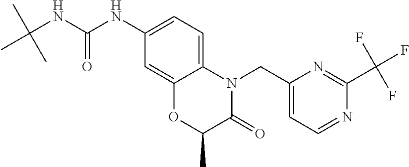 [SMILES: C[C@H]1Oc2cc(NC(=O)NC(C)(C)C)ccc2N(Cc2ccnc(C(F)(F)F)n2)C1=O] | (R)-1-(tert-butyl)-3-(2-methyl-3-oxo-4-((2- (trifluoromethyl)pyrimidin-4-yl)methyl)-3,4-dihydro-2H- benzo[b][1,4]oxazin-7-yl)urea |
| | C₂₀H₂₂F₃N₅O₃ | |
| 168 | 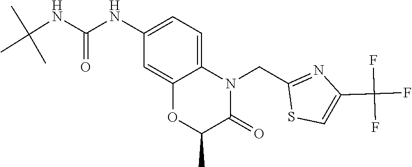 [SMILES: C[C@H]1Oc2cc(NC(=O)NC(C)(C)C)ccc2N(Cc2nc(C(F)(F)F)cs2)C1=O] | (R)-1-(tert-butyl)-3-(2-methyl-3-oxo-4-((4- (trifluoromethyl)thiazol-2-yl)methyl)-3,4-dihydro-2H- benzo[b][1,4]oxazin-7-yl)urea |
| | C₁₉H₂₁F₃N₄O₃S | |
| 169 | 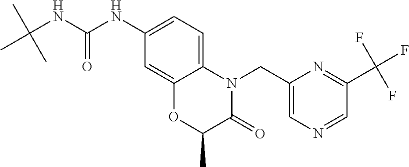 [SMILES: C[C@H]1Oc2cc(NC(=O)NC(C)(C)C)ccc2N(Cc2cncc(C(F)(F)F)n2)C1=O] | (R)-1-(tert-butyl)-3-(2-methyl-3-oxo-4-((6- (trifluoromethyl)pyrazin-2-yl)methyl)-3,4-dihydro-2H- benzo[b][1,4]oxazin-7-yl)urea |
| | C₂₀H₂₂F₃N₅O₃ | |
| 170 | 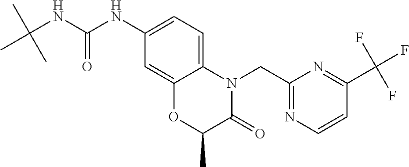 [SMILES: C[C@H]1Oc2cc(NC(=O)NC(C)(C)C)ccc2N(Cc2nccc(C(F)(F)F)n2)C1=O] | (R)-1-(tert-butyl)-3-(2-methyl-3-oxo-4-((4- (trifluoromethyl)pyrimidin-2-yl)methyl)-3,4-dihydro-2H- benzo[b][1,4]oxazin-7-yl)urea |
| | C₂₀H₂₂F₃N₅O₃ | |
| 171 | 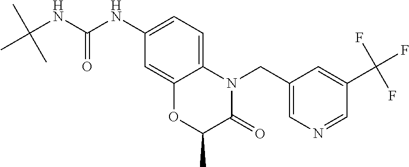 [SMILES: C[C@H]1Oc2cc(NC(=O)NC(C)(C)C)ccc2N(Cc2cncc(C(F)(F)F)c2)C1=O] | (R)-1-(tert-butyl)-3-(2-methyl-3-oxo-4-((5- (trifluoromethyl)pyridin-3-yl)methyl)-3,4-dihydro-2H- benzo[b][1,4]oxazin-7-yl)urea |
| | C₂₁H₂₃F₃N₄O₃ | |
| 172 | 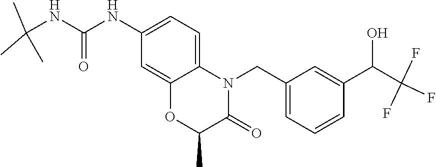 [SMILES: C[C@H]1Oc2cc(NC(=O)NC(C)(C)C)ccc2N(Cc2cccc(C(O)C(F)(F)F)c2)C1=O] | 1-(tert-butyl)-3-((2R)-2-methyl-3-oxo-4-(3-(2,2,2- trifluoro-1-hydroxyethyl)benzyl)-3,4-dihydro-2H- benzo[b][1,4]oxazin-7-yl)urea |
| | C₂₃H₂₆F₃N₃O₄ | |
| 173 | 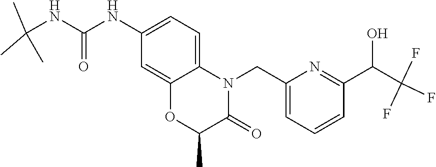 [SMILES: C[C@H]1Oc2cc(NC(=O)NC(C)(C)C)ccc2N(Cc2cccc(C(O)C(F)(F)F)n2)C1=O] | 1-(tert-butyl)-3-((2R)-2-methyl-3-oxo-4-((6-(2,2,2- trifluoro-1-hydroxyethyl)pyridin-2-yl)methyl)-3,4- dihydro-2H-benzo[b][1,4]oxazin-7-yl)urea |
| | C₂₂H₂₅F₃N₄O₄ | |
| 174 | 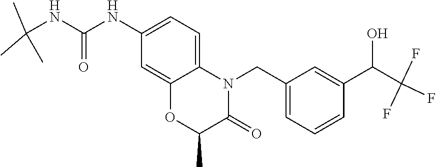 [SMILES: C[C@H]1Oc2cc(NC(=O)NC(C)(C)C)ccc2N(Cc2cccc(C(O)C(F)(F)F)c2)C1=O] | 1-(tert-butyl)-3-((R)-2-methyl-3-oxo-4-(3-((R)-2,2,2- trifluoro-1-hydroxyethyl)benzyl)-3,4-dihydro-2H- benzo[b][1,4]oxazin-7-yl)urea |
| | C₂₃H₂₆F₃N₃O₄ | |
| 175 | 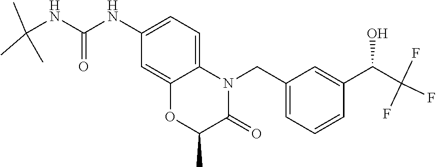 [SMILES: C[C@H]1Oc2cc(NC(=O)NC(C)(C)C)ccc2N(Cc2cccc([C@H](O)C(F)(F)F)c2)C1=O] | 1-(tert-butyl)-3-((R)-2-methyl-3-oxo-4-(3-((S)-2,2,2- trifluoro-1-hydroxyethyl)benzyl)-3,4-dihydro-2H- benzo[b][1,4]oxazin-7-yl)urea |
| | C₂₃H₂₆F₃N₃O₄ | |
| 176 | 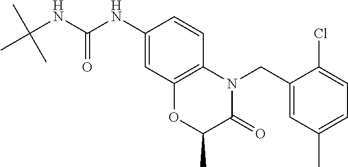 [SMILES: Cc1ccc(Cl)c(CN2C(=O)[C@@H](C)Oc3cc(NC(=O)NC(C)(C)C)ccc32)c1] | (R)-1-(tert-butyl)-3-(4-(2,5-dichlorobenzyl)-2-methyl-3- oxo-3,4-dihydro-2H-benzo[b][1,4]oxazin-7-yl)urea |
| | C₂₁H₂₃Cl₂N₃O₃ | |
| 177 | 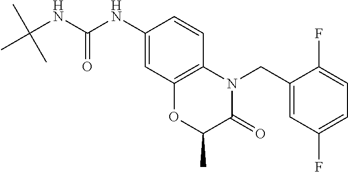 [SMILES: C[C@H]1Oc2cc(NC(=O)NC(C)(C)C)ccc2N(Cc2cc(F)ccc2F)C1=O] | (R)-1-(tert-butyl)-3-(4-(2,5-difluorobenzyl)-2-methyl-3- oxo-3,4-dihydro-2H-benzo[b][1,4]oxazin-7-yl)urea |
| | C₂₁H₂₃F₂N₃O₃ | |
| 178 | 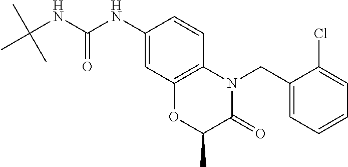 [SMILES: C[C@H]1Oc2cc(NC(=O)NC(C)(C)C)ccc2N(Cc2ccccc2Cl)C1=O] | (R)-1-(tert-butyl)-3-(4-(2-chlorobenzyl)-2-methyl-3-oxo- 3,4-dihydro-2H-benzo[b][1,4]oxazin-7-yl)urea |
| | C₂₁H₂₄ClN₃O₃ | |
| 179 | 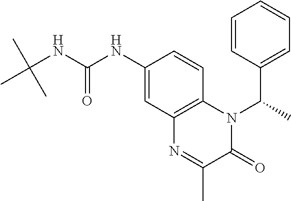 [SMILES: Cc1nc2cc(NC(=O)NC(C)(C)C)ccc2n([C@@H](C)c2ccccc2)c1=O] | (S)-1-(tert-butyl)-3-(3-methyl-2-oxo-1-(1-phenylethyl)- 1,2-dihydroquinoxalin-6-yl)urea |
| | C₂₂H₂₆N₄O₂ | |
| 180 | 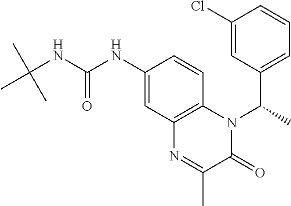 [SMILES: Cc1nc2cc(NC(=O)NC(C)(C)C)ccc2n([C@@H](C)c2cccc(Cl)c2)c1=O] | (S)-1-(tert-butyl)-3-(1-(1-(3-chlorophenyl)ethyl)-3- methyl-2-oxo-1,2-dihydroquinoxalin-6-yl)urea |
| | C₂₂H₂₅ClN₄O₂ | |
| 181 | 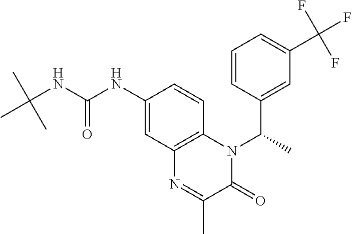 [SMILES: Cc1nc2cc(NC(=O)NC(C)(C)C)ccc2n([C@@H](C)c2cccc(C(F)(F)F)c2)c1=O] | (S)-1-(tert-butyl)-3-(3-methyl-2-oxo-1-(1-(3- (trifluoromethyl)phenyl)ethyl)-1,2-dihydroquinoxalin-6- yl)urea |
| | C₂₃H₂₅F₃N₄O₂ | |
| 182 | 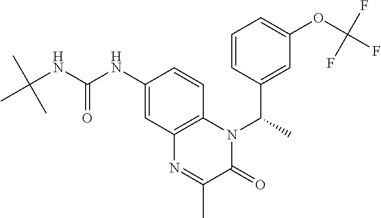 [SMILES: Cc1nc2cc(NC(=O)NC(C)(C)C)ccc2n([C@@H](C)c2cccc(OC(F)(F)F)c2)c1=O] | (S)-1-(tert-butyl)-3-(3-methyl-2-oxo-1-(1-(3- (trifluoromethoxy)phenyl)ethyl)-1,2-dihydroquinoxalin-6- yl)urea |
| | C₂₃H₂₅F₃N₄O₃ | |
| 183 | 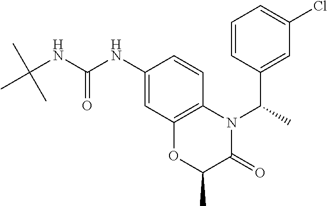 [SMILES: C[C@H]1Oc2cc(NC(=O)NC(C)(C)C)ccc2N([C@@H](C)c2cccc(Cl)c2)C1=O] | 1-(tert-butyl)-3-((R)-4-((S)-1-(3-chlorophenyl)ethyl)-2- methyl-3-oxo-3,4-dihydro-2H-benzo[b][1,4]oxazin-7- yl)urea |
| | C₂₂H₂₆ClN₃O₃ | |
| 184 | 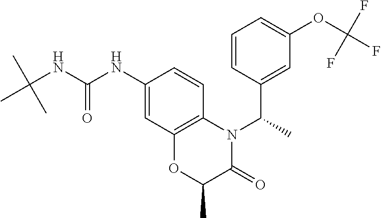 [SMILES: C[C@H]1Oc2cc(NC(=O)NC(C)(C)C)ccc2N([C@@H](C)c2cccc(OC(F)(F)F)c2)C1=O] | 1-(tert-butyl)-3-((R)-2-methyl-3-oxo-4-((S)-1-(3- (trifluoromethoxy)phenyl)ethyl)-3,4-dihydro-2H- benzo[b][1,4]oxazin-7-yl)urea |
| | C₂₃H₂₆F₃N₃O₄ | |
| 185 | 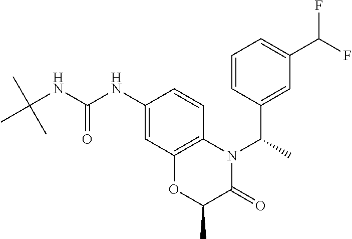 [SMILES: C[C@H]1Oc2cc(NC(=O)NC(C)(C)C)ccc2N([C@@H](C)c2cccc(C(F)F)c2)C1=O] | 1-(tert-butyl)-3-((R)-4-((S)-1-(3- (difluoromethyl)phenyl)ethyl)-2-methyl-3-oxo-3,4- dihydro-2H-benzo[b][1,4]oxazin-7-yl)urea |
| | C₂₃H₂₇F₂N₃O₃ | |
| 186 | 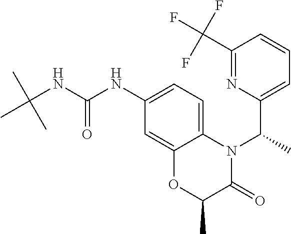 [SMILES: C[C@H]1Oc2cc(NC(=O)NC(C)(C)C)ccc2N([C@@H](C)c2cccc(C(F)(F)F)n2)C1=O] | 1-(tert-butyl)-3-((R)-2-methyl-3-oxo-4-((S)-1-(6- (trifluoromethyl)pyridin-2-yl)ethyl)-3,4-dihydro-2H- benzo[b][1,4]oxazin-7-yl)urea |
| | C₂₂H₂₅F₃N₄O₃ | |
| 187 | 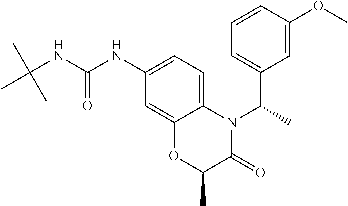 [SMILES: COc1cccc([C@H](C)N2C(=O)[C@@H](C)Oc3cc(NC(=O)NC(C)(C)C)ccc32)c1] | 1-(tert-butyl)-3-((R)-4-((S)-1-(3-methoxyphenyl)ethyl)-2- methyl-3-oxo-3,4-dihydro-2H-benzo[b][1,4]oxazin-7- yl)urea |
| | C₂₃H₂₉N₃O₄ | |
| 188 | 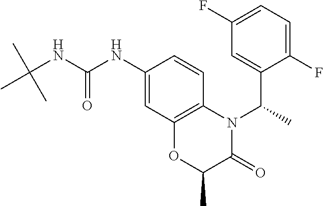 [SMILES: C[C@H]1Oc2cc(NC(=O)NC(C)(C)C)ccc2N([C@@H](C)c2cc(F)ccc2F)C1=O] | 1-(tert-butyl)-3-((R)-4-((S)-1-(2,5-difluorophenyl)ethyl)- 2-methyl-3-oxo-3,4-dihydro-2H-benzo[b][1,4]oxazin-7- yl)urea |
| | C₂₂H₂₅F₂N₃O₃ | |
| 189 | 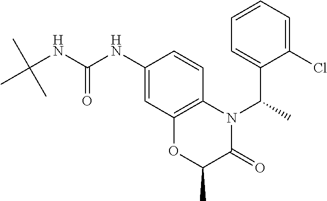 [SMILES: C[C@H]1Oc2cc(NC(=O)NC(C)(C)C)ccc2N([C@@H](C)c2ccccc2Cl)C1=O] | 1-(tert-butyl)-3-((R)-4-((S)-1-(2-chlorophenyl)ethyl)-2- methyl-3-oxo-3,4-dihydro-2H-benzo[b][1,4]oxazin-7- yl)urea |
| | C₂₂H₂₆ClN₃O₃ | |
| 190 | 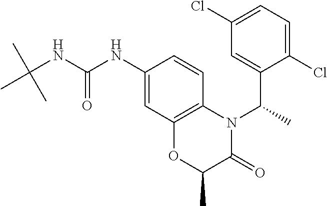 [SMILES: C[C@H]1Oc2cc(NC(=O)NC(C)(C)C)ccc2N([C@@H](C)c2cc(Cl)ccc2Cl)C1=O] | 1-(tert-butyl)-3-((R)-4-((S)-1-(2,5-dichlorophenyl)ethyl)- 2-methyl-3-oxo-3,4-dihydro-2H-benzo[b][1,4]oxazin-7- yl)urea |
| | C₂₂H₂₅Cl₂N₃O₃ | |
| 191 | 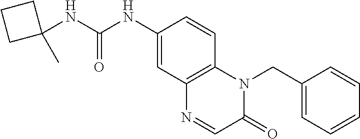 [SMILES: CC1(NC(=O)Nc2ccc3c(c2)ncc(=O)n3Cc2ccccc2)CCC1] | 1-(1-benzyl-2-oxo-1,2-dihydroquinoxalin-6-yl)-3-(1- methylcyclobutyl)urea |
| | C₂₁H₂₂N₄O₂ | |
| 192 | 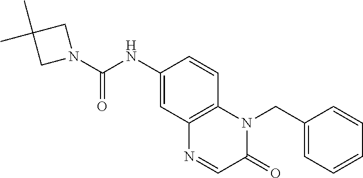 [SMILES: CC1(C)CN(C(=O)Nc2ccc3c(c2)ncc(=O)n3Cc2ccccc2)C1] | N-(1-benzyl-2-oxo-1,2-dihydroquinoxalin-6-yl)-3,3- dimethylazetidine-1-carboxamide |
| | C₂₁H₂₂N₄O₂ | |
| 193 | 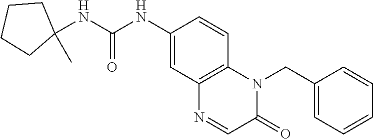 [SMILES: CC1(NC(=O)Nc2ccc3c(c2)ncc(=O)n3Cc2ccccc2)CCCC1] | 1-(1-benzyl-2-oxo-1,2-dihydroquinoxalin-6-yl)-3-(1- methylcyclopentyl)urea |
| | C₂₂H₂₄N₄O₂ | |
| 194 | 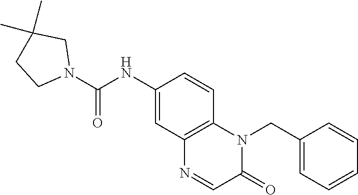 [SMILES: CC1(C)CCN(C(=O)Nc2ccc3c(c2)ncc(=O)n3Cc2ccccc2)C1] | N-(1-benzyl-2-oxo-1,2-dihydroquinoxalin-6-yl)-3,3- dimethylpyrrolidine-1-carboxamide |
| | C₂₂H₂₄N₄O₂ | |
| 195 | 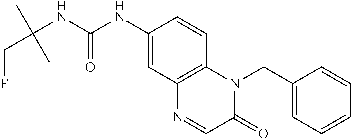 [SMILES: CC(C)(CF)NC(=O)Nc1ccc2c(c1)ncc(=O)n2Cc1ccccc1] | 1-(1-benzyl-2-oxo-1,2-dihydroquinoxalin-6-yl)-3-(1- fluoro-2-methylpropan-2-yl)urea |
| | C₂₀H₂₁FN₄O₂ | |
| 196 | 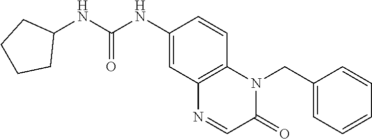 [SMILES: O=C(Nc1ccc2c(c1)ncc(=O)n2Cc1ccccc1)NC1CCCC1] | 1-(1-benzyl-2-oxo-1,2-dihydroquinoxalin-6-yl)-3- cyclopentylurea |
| | C₂₁H₂₂N₄O₂ | |
| 197 | 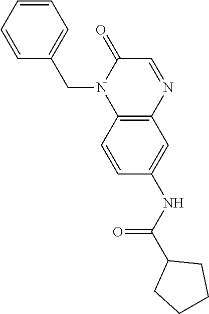 [SMILES: O=C(Nc1ccc2c(c1)ncc(=O)n2Cc1ccccc1)C1CCCC1] | N-(1-benzyl-2-oxo-1,2-dihydroquinoxalin-6- yl)cyclopentanecarboxamide |
| | C₂₁H₂₁N₃O₂ | |
| 198 | 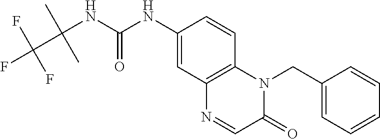 [SMILES: CC(C)(NC(=O)Nc1ccc2c(c1)ncc(=O)n2Cc1ccccc1)C(F)(F)F] | 1-(1-benzyl-2-oxo-1,2-dihydroquinoxalin-6-yl)-3-(1,1,1- trifluoro-2-methylpropan-2-yl)urea |
| | C₂₀H₁₉F₃N₄O₂ | |
| 199 | 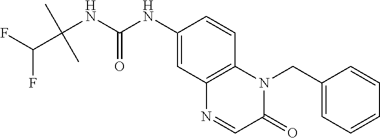 [SMILES: CC(C)(NC(=O)Nc1ccc2c(c1)ncc(=O)n2Cc1ccccc1)C(F)F] | 1-(1-benzyl-2-oxo-1,2-dihydroquinoxalin-6-yl)-3-(1,1- difluoro-2-methylpropan-2-yl)urea |
| | C₂₀H₂₀F₂N₄O₂ | |
| 200 | 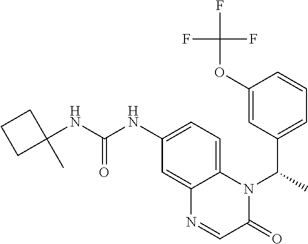 [SMILES: C[C@@H](c1cccc(OC(F)(F)F)c1)n1c(=O)cnc2cc(NC(=O)NC3(C)CCC3)ccc21] | (S)-1-(1-methylcyclobutyl)-3-(2-oxo-1-(1-(3- (trifluoromethoxy)phenyl)ethyl)-1,2-dihydroquinoxalin-6- yl)urea |
| | C₂₃H₂₃F₃N₄O₃ | |
| 201 | 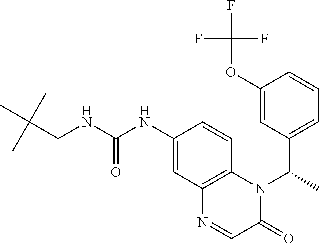 [SMILES: C[C@@H](c1cccc(OC(F)(F)F)c1)n1c(=O)cnc2cc(NC(=O)NCC(C)(C)C)ccc21] | (S)-1-neopentyl-3-(2-oxo-1-(1-(3- (trifluoromethoxy)phenyl)ethyl)-1,2-dihydroquinoxalin-6- yl)urea |
| | C₂₃H₂₅F₃N₄O₃ | |
| 202 | 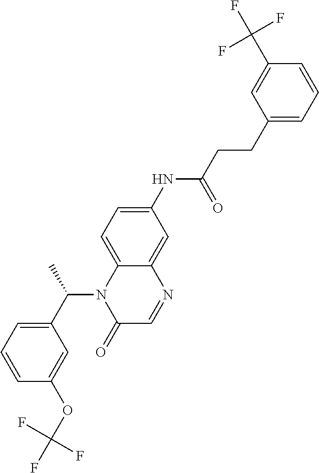 [SMILES: C[C@@H](c1cccc(OC(F)(F)F)c1)n1c(=O)cnc2cc(NC(=O)CCc3cccc(C(F)(F)F)c3)ccc21] | (S)-N-(2-oxo-1-(1-(3-(trifluoromethoxy)phenyl)ethyl)- 1,2-dihydroquinoxalin-6-yl)-3-(3- (trifluoromethyl)phenyl)propanamide |
| | C₂₇H₂₁F₆N₃O₃ | |
| 203 | 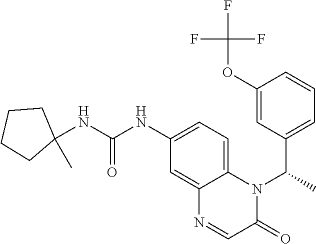 [SMILES: C[C@@H](c1cccc(OC(F)(F)F)c1)n1c(=O)cnc2cc(NC(=O)NC3(C)CCCC3)ccc21] | (S)-1-(1-methylcyclopentyl)-3-(2-oxo-1-(1-(3- (trifluoromethoxy)phenyl)ethyl)-1,2-dihydroquinoxalin-6- yl)urea |
| | C₂₄H₂₅F₃N₄O₃ | |
| 204 | 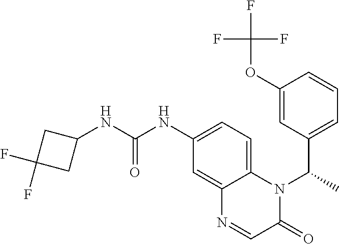 [SMILES: C[C@@H](c1cccc(OC(F)(F)F)c1)n1c(=O)cnc2cc(NC(=O)NC3CC(F)(F)C3)ccc21] | (S)-1-(3,3-difluorocyclobutyl)-3-(2-oxo-1-(1-(3- (trifluoromethoxy)phenyl)ethyl)-1,2-dihydroquinoxalin-6- yl)urea |
| | C₂₂H₁₉F₅N₄O₃ | |
| 205 | 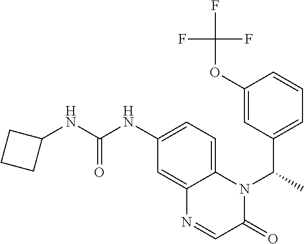 [SMILES: C[C@@H](c1cccc(OC(F)(F)F)c1)n1c(=O)cnc2cc(NC(=O)NC3CCC3)ccc21] | (S)-1-cyclobutyl-3-(2-oxo-1-(1-(3- (trifluoromethoxy)phenyl)ethyl)-1,2-dihydroquinoxalin-6- yl)urea |
| | C₂₂H₂₁F₃N₄O₃ | |
| 206 | 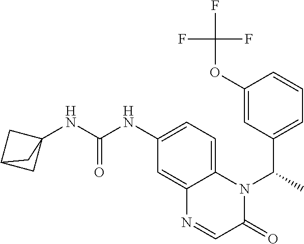 [SMILES: C[C@@H](c1cccc(OC(F)(F)F)c1)n1c(=O)cnc2cc(NC(=O)NC34CC(C3)C4)ccc21] | (S)-1-(bicyclo[1.1.1]pentan-1-yl)-3-(2-oxo-1-(1-(3- (trifluoromethoxy)phenyl)ethyl)-1,2-dihydroquinoxalin-6- yl)urea |
| | C₂₃H₂₁F₃N₄O₃ | |
| 207 | 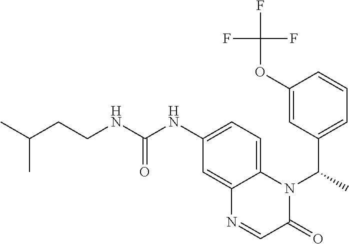 [SMILES: CC(C)CCNC(=O)Nc1ccc2c(c1)ncc(=O)n2[C@@H](C)c1cccc(OC(F)(F)F)c1] | (S)-1-isopentyl-3-(2-oxo-1-(1-(3- (trifluoromethoxy)phenyl)ethyl)-1,2-dihydroquinoxalin-6- yl)urea |
| | C₂₃H₂₅F₃N₄O₃ | |
| 208 | 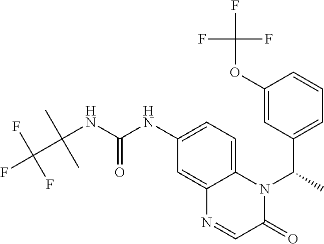 [SMILES: C[C@@H](c1cccc(OC(F)(F)F)c1)n1c(=O)cnc2cc(NC(=O)NC(C)(C)C(F)(F)F)ccc21] | (S)-1-(2-oxo-1-(1-(3-(trifluoromethoxy)phenyl)ethyl)-1,2- dihydroquinoxalin-6-yl)-3-(1,1,1-trifluoro-2- methylpropan-2-yl)urea |
| | C₂₂H₂₀F₆N₄O₃ | |
| 209 | 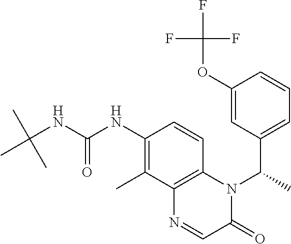 [SMILES: Cc1c(NC(=O)NC(C)(C)C)ccc2c1ncc(=O)n2[C@@H](C)c1cccc(OC(F)(F)F)c1] | (S)-1-(tert-butyl)-3-(5-methyl-2-oxo-1-(1-(3- (trifluoromethoxy)phenyl)ethyl)-1,2-dihydroquinoxalin-6- yl)urea |
| | C₂₃H₂₅F₃N₄O₃ | |
| 210 | 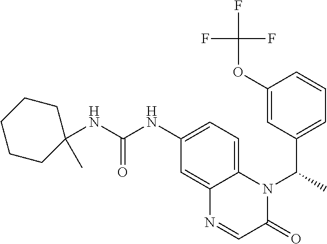 [SMILES: C[C@@H](c1cccc(OC(F)(F)F)c1)n1c(=O)cnc2cc(NC(=O)NC3(C)CCCCC3)ccc21] | (S)-1-(1-methylcyclohexyl)-3-(2-oxo-1-(1-(3- (trifluoromethoxy)phenyl)ethyl)-1,2-dihydroquinoxalin-6- yl)urea |
| | C₂₅H₂₇F₃N₄O₃ | |
| 211 | 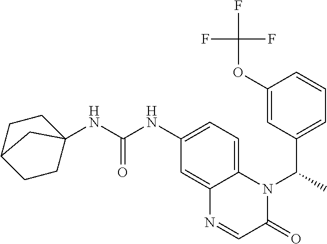 [SMILES: C[C@@H](c1cccc(OC(F)(F)F)c1)n1c(=O)cnc2cc(NC(=O)NC34CCC(CC3)C4)ccc21] | (S)-1-(bicyclo[2.2.1]heptan-1-yl)-3-(2-oxo-1-(1-(3- (trifluoromethoxy)phenyl)ethyl)-1,2-dihydroquinoxalin-6- yl)urea |
| | C₂₅H₂₅F₃N₄O₃ | |
| 212 | 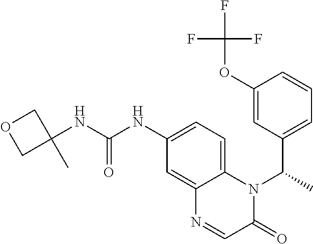 [SMILES: C[C@@H](c1cccc(OC(F)(F)F)c1)n1c(=O)cnc2cc(NC(=O)NC3(C)COC3)ccc21] | (S)-1-(3-methyloxetan-3-yl)-3-(2-oxo-1-(1-(3- (trifluoromethoxy)phenyl)ethyl)-1,2-dihydroquinoxalin-6- yl)urea |
| | C₂₂H₂₁F₃N₄O₄ | |
| 213 | 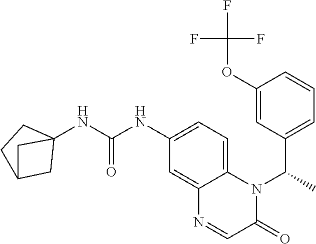 [SMILES: C[C@@H](c1cccc(OC(F)(F)F)c1)n1c(=O)cnc2cc(NC(=O)NC34CCC(C3)C4)ccc21] | (S)-1-(bicyclo[2.1.1]hexan-1-yl)-3-(2-oxo-1-(1-(3- (trifluoromethoxy)phenyl)ethyl)-1,2-dihydroquinoxalin-6- yl)urea |
| | C₂₄H₂₃F₃N₄O₃ | |
| 214 | 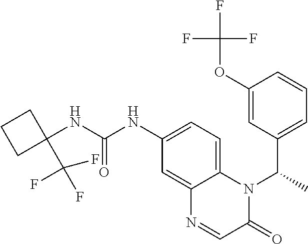 [SMILES: C[C@@H](c1cccc(OC(F)(F)F)c1)n1c(=O)cnc2cc(NC(=O)NC3(C(F)(F)F)CCC3)ccc21] | (S)-1-(2-oxo-1-(1-(3-(trifluoromethoxy)phenyl)ethyl)-1,2- dihydroquinoxalin-6-yl)-3-(1- (trifluoromethyl)cyclobutyl)urea |
| | C₂₃H₂₀F₆N₄O₃ | |
| 215 | 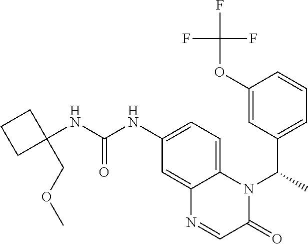 [SMILES: COCC1(NC(=O)Nc2ccc3c(c2)ncc(=O)n3[C@@H](C)c2cccc(OC(F)(F)F)c2)CCC1] | (S)-1-(1-(methoxymethyl)cyclobutyl)-3-(2-oxo-1-(1-(3- (trifluoromethoxy)phenyl)ethyl)-1,2-dihydroquinoxalin-6- yl)urea |
| | C₂₄H₂₅F₃N₄O₄ | |
| 216 | 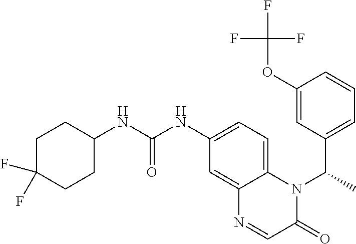 [SMILES: C[C@@H](c1cccc(OC(F)(F)F)c1)n1c(=O)cnc2cc(NC(=O)NC3CCC(F)(F)CC3)ccc21] | (S)-1-(4,4-difluorocyclohexyl)-3-(2-oxo-1-(1-(3- (trifluoromethoxy)phenyl)ethyl)-1,2-dihydroquinoxalin-6- yl)urea |
| | C₂₄H₂₃F₅N₄O₃ | |
| 217 | 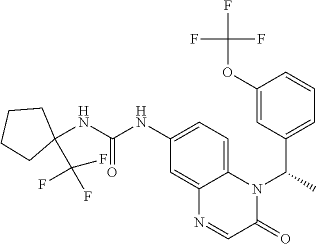 [SMILES: C[C@@H](c1cccc(OC(F)(F)F)c1)n1c(=O)cnc2cc(NC(=O)NC3(C(F)(F)F)CCCC3)ccc21] | (S)-1-(2-oxo-1-(1-(3-(trifluoromethoxy)phenyl)ethyl)-1,2- dihydroquinoxalin-6-yl)-3-(1- (trifluoromethyl)cyclopentyl)urea |
| | C₂₄H₂₂F₆N₄O₃ | |
| 218 | 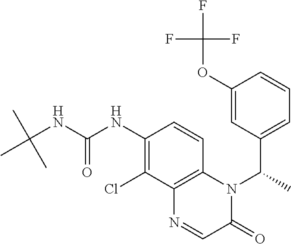 [SMILES: C[C@@H](c1cccc(OC(F)(F)F)c1)n1c(=O)cnc2c(Cl)c(NC(=O)NC(C)(C)C)ccc21] | (S)-1-(tert-butyl)-3-(5-chloro-2-oxo-1-(1-(3- (trifluoromethoxy)phenyl)ethyl)-1,2-dihydroquinoxalin-6- yl)urea |
| | C₂₂H₂₂ClF₃N₄O₃ | |
| 219 | 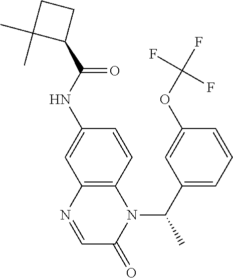 [SMILES: C[C@@H](c1cccc(OC(F)(F)F)c1)n1c(=O)cnc2cc(NC(=O)[C@@H]3CCC3(C)C)ccc21] | (R)-2,2-dimethyl-N-(2-oxo-1-((S)-1-(3- (trifluoromethoxy)phenyl)ethyl)-1,2-dihydroquinoxalin-6- yl)cyclobutane-1-carboxamide |
| | C₂₄H₂₄F₃N₃O₃ | |
| 220 | 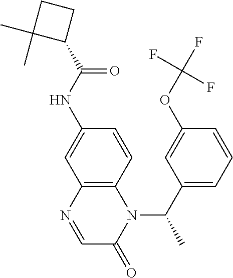 [SMILES: C[C@@H](c1cccc(OC(F)(F)F)c1)n1c(=O)cnc2cc(NC(=O)[C@H]3CCC3(C)C)ccc21] | (S)-2,2-dimethyl-N-(2-oxo-1-((S)-1-(3- (trifluoromethoxy)phenyl)ethyl)-1,2-dihydroquinoxalin-6- yl)cyclobutane-1-carboxamide |
| | C₂₄H₂₄F₃N₃O₃ | |
| 221 | 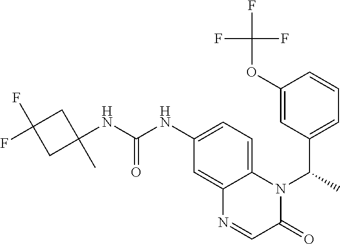 [SMILES: C[C@@H](c1cccc(OC(F)(F)F)c1)n1c(=O)cnc2cc(NC(=O)NC3(C)CC(F)(F)C3)ccc21] | (S)-1-(3,3-difluoro-1-methylcyclobutyl)-3-(2-oxo-1-(1-(3- (trifluoromethoxy)phenyl)ethyl)-1,2-dihydroquinoxalin-6- yl)urea |
| | C₂₃H₂₁F₅N₄O₃ | |
| 222 | 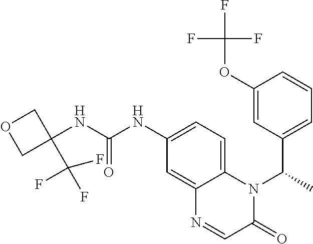 [SMILES: C[C@@H](c1cccc(OC(F)(F)F)c1)n1c(=O)cnc2cc(NC(=O)NC3(C(F)(F)F)COC3)ccc21] | (S)-1-(2-oxo-1-(1-(3-(trifluoromethoxy)phenyl)ethyl)-1,2- dihydroquinoxalin-6-yl)-3-(3-(trifluoromethyl)oxetan-3- yl)urea |
| | C2₂H₁₈F₆N₄O₄ | |
| 223 | 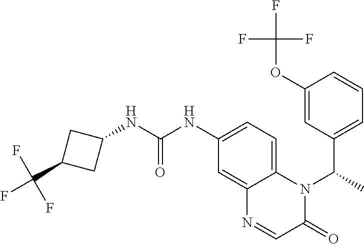 [SMILES: C[C@@H](c1cccc(OC(F)(F)F)c1)n1c(=O)cnc2cc(NC(=O)N[C@H]3C[C@H](C(F)(F)F)C3)ccc21] | 1-(2-oxo-1-((S)-1-(3-(trifluoromethoxy)phenyl)ethyl)-1,2- dihydroquinoxalin-6-yl)-3-((1r,3S)-3- (trifluoromethyl)cyclobutyl)urea |
| | C₂₃H₂₀F₆N₄O₃ | |
| 224 | 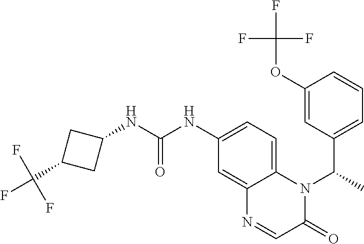 [SMILES: C[C@@H](c1cccc(OC(F)(F)F)c1)n1c(=O)cnc2cc(NC(=O)N[C@H]3C[C@@H](C(F)(F)F)C3)ccc21] | 1-(2-oxo-1-((S)-1-(3-(trifluoromethoxy)phenyl)ethyl)-1,2- dihydroquinoxalin-6-yl)-3-((1s,3R)-3- (trifluoromethyl)cyclobutyl)urea |
| | C₂₃H₂₀F₆N₄O₃ | |
| 225 | 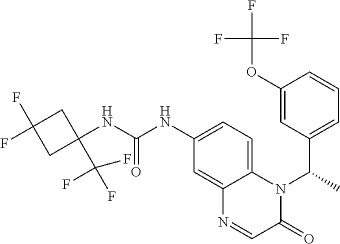 [SMILES: C[C@@H](c1cccc(OC(F)(F)F)c1)n1c(=O)cnc2cc(NC(=O)NC3(C(F)(F)F)CC(F)(F)C3)ccc21] | (S)-1-(3,3-difluoro-1-(trifluoromethyl)cyclobutyl)-3-(2- oxo-1-(1-(3-(trifluoromethoxy)phenyl)ethyl)-1,2- dihydroquinoxalin-6-yl)urea |
| | C₂₃H₁₈F₈N₄O₃ | |
| 226 | 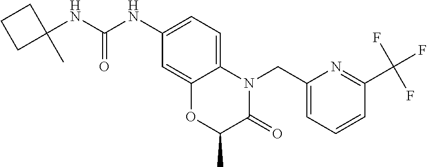 [SMILES: C[C@H]1Oc2cc(NC(=O)NC3(C)CCC3)ccc2N(Cc2cccc(C(F)(F)F)n2)C1=O] | (R)-1-(2-methyl-3-oxo-4-((6-(trifluoromethyl)pyridin-2- yl)methyl)-3,4-dihydro-2H-benzo[b][1,4]oxazin-7-yl)-3- (1-methylcyclobutyl)urea |
| | C₂₂H₂₃F₃N₄O₃ | |
| 227 | 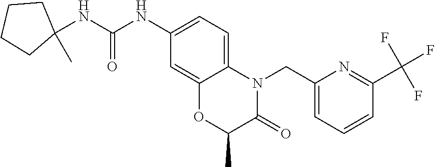 [SMILES: C[C@H]1Oc2cc(NC(=O)NC3(C)CCCC3)ccc2N(Cc2cccc(C(F)(F)F)n2)C1=O] | (R)-1-(2-methyl-3-oxo-4-((6-(trifluoromethyl)pyridin-2- yl)methyl)-3,4-dihydro-2H-benzo[b][1,4]oxazin-7-yl)-3- (1-methylcyclopentyl)urea |
| | C₂₃H₂₅F₃N₄O₃ | |
| 228 | 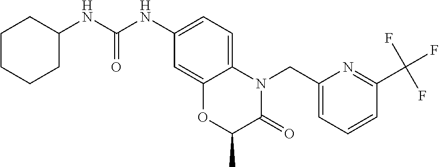 [SMILES: C[C@H]1Oc2cc(NC(=O)NC3CCCCC3)ccc2N(Cc2cccc(C(F)(F)F)n2)C1=O] | (R)-1-cyclohexyl-3-(2-methyl-3-oxo-4-((6- (trifluoromethyl)pyridin-2-yl)methyl)-3,4-dihydro-2H- benzo[b][1,4]oxazin-7-yl)urea |
| | C₂₃H₂₅F₃N₄O₃ | |
| 229 | 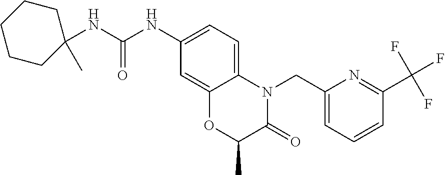 [SMILES: C[C@H]1Oc2cc(NC(=O)NC3(C)CCCCC3)ccc2N(Cc2cccc(C(F)(F)F)n2)C1=O] | (R)-1-(2-methyl-3-oxo-4-((6-(trifluoromethyl)pyridin-2- yl)methyl)-3,4-dihydro-2H-benzo[b][1,4]oxazin-7-yl)-3- (1-methylcyclohexyl)urea |
| | C₂₄H₂₇F₃N₄O₃ | |
| 230 | 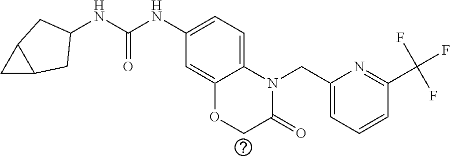 [SMILES: O=C(Nc1ccc2c(c1)OCC(=O)N2Cc1cccc(C(F)(F)F)n1)NC1CC2CC2C1] | 1-(bicyclo[3.1.0]hexan-3-yl)-3-((R)-2-methyl-3-oxo-4- ((6-(trifluoromethyl)pyridin-2-yl)methyl)-3,4-dihydro- 2H-benzo[b][1,4]oxazin-7-yl)urea |
| | C₂₃H₂₃F₃N₄O₃ | |
| 231 | 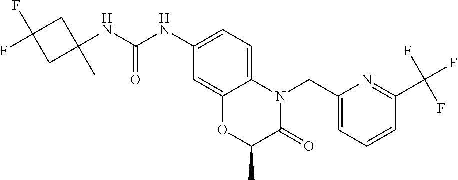 [SMILES: C[C@H]1Oc2cc(NC(=O)NC3(C)CC(F)(F)C3)ccc2N(Cc2cccc(C(F)(F)F)n2)C1=O] | (R)-1-(3,3-difluoro-1-methylcyclobutyl)-3-(2-methyl-3- oxo-4-((6-(trifluoromethyl)pyridin-2-yl)methyl)-3,4- dihydro-2H-benzo[b][1,4]oxazin-7-yl)urea |
| | C₂₂H₂₁F₅N₄O₃ | |
| 232 | 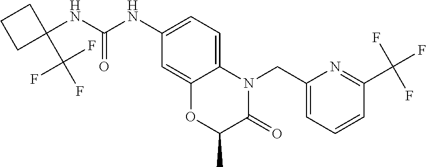 [SMILES: C[C@H]1Oc2cc(NC(=O)NC3(C(F)(F)F)CCC3)ccc2N(Cc2cccc(C(F)(F)F)n2)C1=O] | (R)-1-(2-methyl-3-oxo-4-((6-(trifluoromethyl)pyridin-2- yl)methyl)-3,4-dihydro-2H-benzo[b][1,4]oxazin-7-yl)-3- (1-(trifluoromethyl)cyclobutyl)urea |
| | C₂₂H₂₀F₆N₄O₃ | |
| 233 | 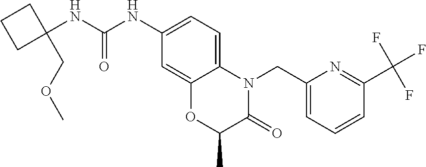 [SMILES: COCC1(NC(=O)Nc2ccc3c(c2)O[C@H](C)C(=O)N3Cc2cccc(C(F)(F)F)n2)CCC1] | (R)-1-(1-(methoxymethyl)cyclobutyl)-3-(2-methyl-3-oxo- 4-((6-(trifluoromethyl)pyridin-2-yl)methyl)-3,4-dihydro- 2H-benzo[b][1,4]oxazin-7-yl)urea |
| | C₂₃H₂₅F₃N₄O₄ | |
| 234 | 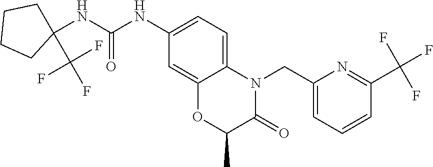 [SMILES: C[C@H]1Oc2cc(NC(=O)NC3(C(F)(F)F)CCCC3)ccc2N(Cc2cccc(C(F)(F)F)n2)C1=O] | (R)-1-(2-methyl-3-oxo-4-((6-(trifluoromethyl)pyridin-2- yl)methyl)-3,4-dihydro-2H-benzo[b][1,4]oxazin-7-yl)-3- (1-(trifluoromethyl)cyclopentyl)urea |
| | C₂₃H₂₂F₆N₄O₃ | |
| 235 | 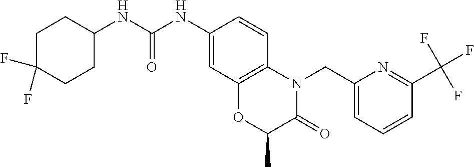 [SMILES: C[C@H]1Oc2cc(NC(=O)NC3CCC(F)(F)CC3)ccc2N(Cc2cccc(C(F)(F)F)n2)C1=O] | (R)-1-(4,4-difluorocyclohexyl)-3-(2-methyl-3-oxo-4-((6- (trifluoromethyl)pyridin-2-yl)methyl)-3,4-dihydro-2H- benzo[b][1,4]oxazin-7-yl)urea |
| | C₂₃H₂₃F₅N₄O₃ | |
| 236 | 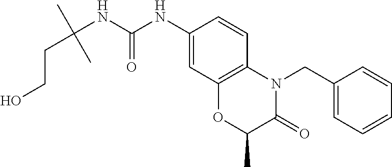 [SMILES: C[C@H]1Oc2cc(NC(=O)NC(C)(C)CCO)ccc2N(Cc2ccccc2)C1=O] | (R)-1-(4-benzyl-2-methyl-3-oxo-3,4-dihydro-2H- benzo[b][1,4]oxazin-7-yl)-3-(4-hydroxy-2-methylbutan- 2-yl)urea |
| | C₂₂H₂₇N₃O₄ | |
| 237 | 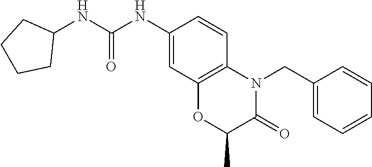 [SMILES: C[C@H]1Oc2cc(NC(=O)NC3CCCC3)ccc2N(Cc2ccccc2)C1=O] | (R)-1-(4-benzyl-2-methyl-3-oxo-3,4-dihydro-2H- benzo[b][1,4]oxazin-7-yl)-3-cyclopentylurea |
| | C₂₂H₂₅N₃O₃ | |
| 238 | 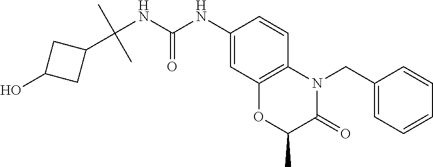 [SMILES: C[C@H]1Oc2cc(NC(=O)NC(C)(C)C3CC(O)C3)ccc2N(Cc2ccccc2)C1=O] | 1-((R)-4-benzyl-2-methyl-3-oxo-3,4-dihydro-2H- benzo[b][1,4]oxazin-7-yl)-3-(2-((1s,3S)-3- hydroxycyclobutyl)propan-2-yl)urea |
| | C₂₄H₂₉N₃O₄ | |
| 239 | 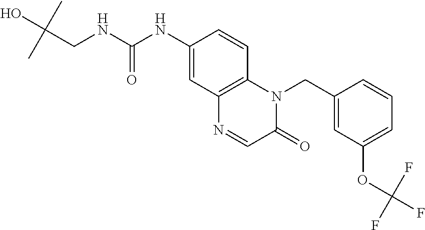 [SMILES: CC(C)(O)CNC(=O)Nc1ccc2c(c1)ncc(=O)n2Cc1cccc(OC(F)(F)F)c1] | 1-(2-hydroxy-2-methylpropyl)-3-(2-oxo-1-(3- (trifluoromethoxy)benzyl)-1,2-dihydroquinoxalin-6- yl)urea |
| | C₂₁H₂₁F₃N₄O₄ | |
| 240 | 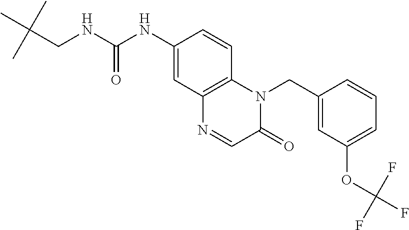 [SMILES: CC(C)(C)CNC(=O)Nc1ccc2c(c1)ncc(=O)n2Cc1cccc(OC(F)(F)F)c1] | 1-neopentyl-3-(2-oxo-1-(3-(trifluoromethoxy)benzyl)-1,2- dihydroquinoxalin-6-yl)urea |
| | C₂₂H₂₃F₃N₄O₃ | |
| 241 | 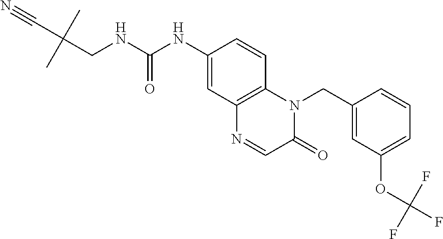 [SMILES: CC(C)(C#N)CNC(=O)Nc1ccc2c(c1)ncc(=O)n2Cc1cccc(OC(F)(F)F)c1] | 1-(2-cyano-2-methylpropyl)-3-(2-oxo-1-(3- (trifluoromethoxy)benzyl)-1,2-dihydroquinoxalin-6- yl)urea |
| | C₂₂H₂₀F₃N₅O₃ | |
| 242 | 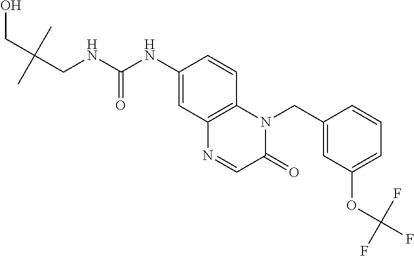 [SMILES: CC(C)(CO)CNC(=O)Nc1ccc2c(c1)ncc(=O)n2Cc1cccc(OC(F)(F)F)c1] | 1-(3-hydroxy-2,2-dimethylpropyl)-3-(2-oxo-1-(3- (trifluoromethoxy)benzyl)-1,2-dihydroquinoxalin-6- yl)urea |
| | C₂₂H₂₃F₃N₄O₄ | |
| 243 | 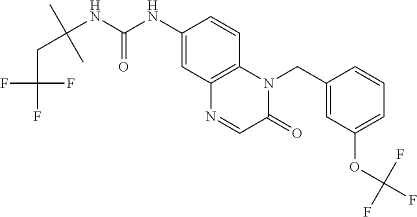 [SMILES: CC(C)(CC(F)(F)F)NC(=O)Nc1ccc2c(c1)ncc(=O)n2Cc1cccc(OC(F)(F)F)c1] | 1-(2-oxo-1-(3-(trifluoromethoxy)benzyl)-1,2- dihydroquinoxalin-6-yl)-3-(4,4,4-trifluoro-2-methylbutan- 2-yl)urea |
| | C₂₂H₂₀F₆N₄O₃ | |
| 244 | 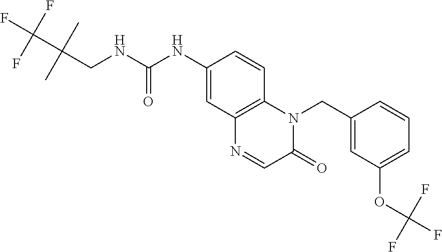 [SMILES: CC(C)(CNC(=O)Nc1ccc2c(c1)ncc(=O)n2Cc1cccc(OC(F)(F)F)c1)C(F)(F)F] | 1-(2-oxo-1-(3-(trifluoromethoxy)benzyl)-1,2- dihydroquinoxalin-6-yl)-3-(3,3,3-trifluoro-2,2- dimethylpropyl)urea |
| | C₂₂H₂₀F₆N₄O₃ | |
| 245 | 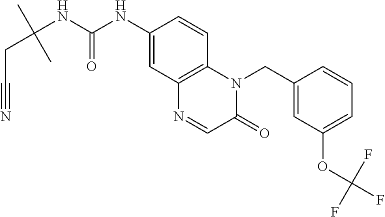 [SMILES: CC(C)(CC#N)NC(=O)Nc1ccc2c(c1)ncc(=O)n2Cc1cccc(OC(F)(F)F)c1] | 1-(1-cyano-2-methylpropan-2-yl)-3-(2-oxo-1-(3- (trifluoromethoxy)benzyl)-1,2-dihydroquinoxalin-6- yl)urea |
| | C₂₂H₂₀F₃N₅O₃ | |
| 246 | 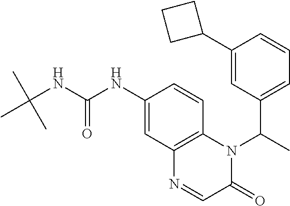 [SMILES: CC(c1cccc(C2CCC2)c1)n1c(=O)cnc2cc(NC(=O)NC(C)(C)C)ccc21] | 1-(tert-butyl)-3-(1-(1-(3-cyclobutylphenyl)ethyl)-2-oxo- 1,2-dihydroquinoxalin-6-yl)urea |
| | C₂₅H₃₀N₄O₂ | |
| 247 | 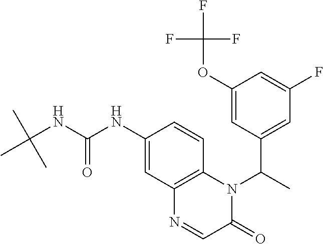 [SMILES: CC(c1cc(F)cc(OC(F)(F)F)c1)n1c(=O)cnc2cc(NC(=O)NC(C)(C)C)ccc21] | 1-(tert-butyl)-3-(1-(1-(3-fluoro-5- (trifluoromethoxy)phenyl)ethyl)-2-oxo-1,2- dihydroquinoxalin-6-yl)urea |
| | C₂₂H₂₂F₄N₄O₃ | |
| 248 | 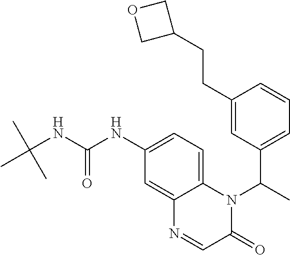 [SMILES: CC(c1cccc(CCC2COC2)c1)n1c(=O)cnc2cc(NC(=O)NC(C)(C)C)ccc21] | 1-(tert-butyl)-3-(1-(1-(3-(2-(oxetan-3- yl)ethyl)phenyl)ethyl)-2-oxo-1,2-dihydroquinoxalin-6- yl)urea |
| | C₂₆H₃₂N₄O₃ | |
| 249 | 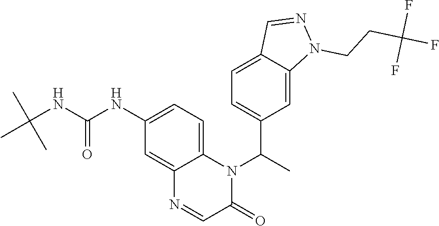 [SMILES: CC(c1ccc2cnn(CCC(F)(F)F)c2c1)n1c(=O)cnc2cc(NC(=O)NC(C)(C)C)ccc21] | 1-(tert-butyl)-3-(2-oxo-1-(1-(1-(3,3,3-trifluoropropyl)-1H- indazol-6-yl)ethyl)-1,2-dihydroquinoxalin-6-yl)urea |
| | C₂₅H₂₇F₃N₆O₂ | |
| 250 | 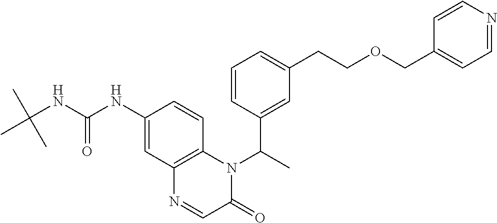 [SMILES: CC(c1cccc(CCOCc2ccncc2)c1)n1c(=O)cnc2cc(NC(=O)NC(C)(C)C)ccc21] | 1-(tert-butyl)-3-(2-oxo-1-(1-(3-(2-(pyridin-4- ylmethoxy)ethyl)phenyl)ethyl)-1,2-dihydroquinoxalin-6- yl)urea |
| | C₂₉H₃₃N₅O₃ | |
| 251 | 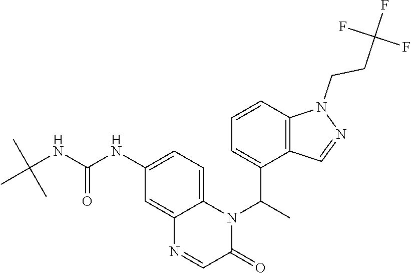 [SMILES: CC(c1cccc2c1cnn2CCC(F)(F)F)n1c(=O)cnc2cc(NC(=O)NC(C)(C)C)ccc21] | 1-(tert-butyl)-3-(2-oxo-1-(1-(1-(3,3,3-trifluoropropyl)-1H- indazol-4-yl)ethyl)-1,2-dihydroquinoxalin-6-yl)urea |
| | C₂₅H₂₇F₃N₆O₂ | |
| 252 | 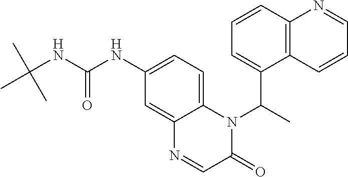 [SMILES: CC(c1cccc2ncccc12)n1c(=O)cnc2cc(NC(=O)NC(C)(C)C)ccc21] | 1-(tert-butyl)-3-(2-oxo-1-(1-(quinolin-5-yl)ethyl)-1,2- dihydroquinoxalin-6-yl)urea |
| | C₂₄H₂₅N₅O₂ | |
| 253 | 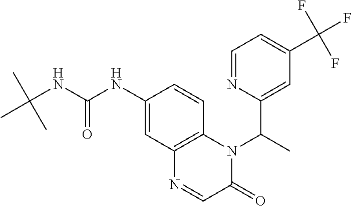 [SMILES: CC(c1cc(C(F)(F)F)ccn1)n1c(=O)cnc2cc(NC(=O)NC(C)(C)C)ccc21] | 1-(tert-butyl)-3-(2-oxo-1-(1-(4-(trifluoromethyl)pyridin-2- yl)ethyl)-1,2-dihydroquinoxalin-6-yl)urea |
| | C₂₁H₂₂F₃N₅O₂ | |
| 254 | 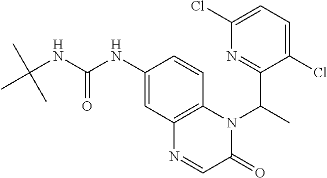 [SMILES: CC(c1nc(Cl)ccc1Cl)n1c(=O)cnc2cc(NC(=O)NC(C)(C)C)ccc21] | 1-(tert-butyl)-3-(1-(1-(3,6-dichloropyridin-2-yl)ethyl)-2- oxo-1,2-dihydroquinoxalin-6-yl)urea |
| | C₂₀H₂₁C₁₂N₅O₂ | |
| 255 | 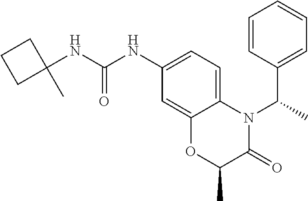 [SMILES: C[C@H]1Oc2cc(NC(=O)NC3(C)CCC3)ccc2N([C@@H](C)c2ccccc2)C1=O] | 1-((R)-2-methyl-3-oxo-4-((S)-1-phenylethyl)-3,4- dihydro-2H-benzo[b][1,4]oxazin-7-yl)-3-(1- methylcyclobutyl)urea |
| | C₂₃H₂₇N₃O₃ | |
| 256 | 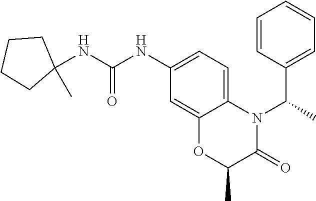 [SMILES: C[C@H]1Oc2cc(NC(=O)NC3(C)CCCC3)ccc2N([C@@H](C)c2ccccc2)C1=O] | 1-((R)-2-methyl-3-oxo-4-((S)-1-phenylethyl)-3,4- dihydro-2H-benzo[b][1,4]oxazin-7-yl)-3-(1- methylcyclopentyl)urea |
| | C₂₄H₂₉N₃O₃ | |
| 257 | 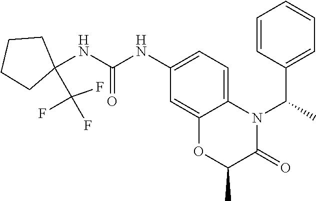 [SMILES: C[C@H]1Oc2cc(NC(=O)NC3(C(F)(F)F)CCCC3)ccc2N([C@@H](C)c2ccccc2)C1=O] | 1-((R)-2-methyl-3-oxo-4-((S)-1-phenylethyl)-3,4- dihydro-2H-benzo[b][1,4]oxazin-7-yl)-3-(1- (trifluoromethyl)cyclopentyl)urea |
| | C₂₄H₂₆F₃N₃O₃ | |
| 258 | 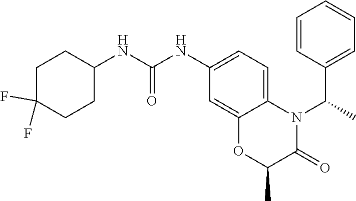 [SMILES: C[C@H]1Oc2cc(NC(=O)NC3CCC(F)(F)CC3)ccc2N([C@@H](C)c2ccccc2)C1=O] | 1-(4,4-difluorocyclohexyl)-3-((R)-2-methyl-3-oxo-4-((S)- 1-phenylethyl)-3,4-dihydro-2H-benzo[b][1,4]oxazin-7- yl)urea |
| | C₂₄H₂₇F₂N₃O₃ | |
| 259 | 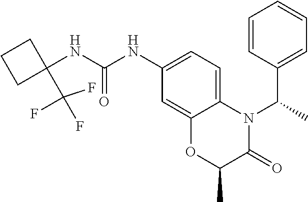 [SMILES: C[C@H]1Oc2cc(NC(=O)NC3(C(F)(F)F)CCC3)ccc2N([C@@H](C)c2ccccc2)C1=O] | 1-((R)-2-methyl-3-oxo-4-((S)-1-phenylethyl)-3,4- dihydro-2H-benzo[b][1,4]oxazin-7-yl)-3-(1- (trifluoromethyl)cyclobutyl)urea |
| | C₂₃H₂₄F₃N₃O₃ | |
| 260 | 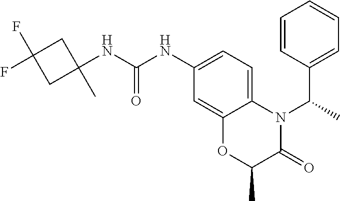 [SMILES: C[C@H]1Oc2cc(NC(=O)NC3(C)CC(F)(F)C3)ccc2N([C@@H](C)c2ccccc2)C1=O] | 1-(3,3-difluoro-1-methylcyclobutyl)-3-((R)-2-methyl-3- oxo-4-((S)-1-phenylethyl)-3,4-dihydro-2H- benzo[b][1,4]oxazin-7-yl)urea |
| | C₂₃H₂₅F₂N₃O₃ | |
| 261 | 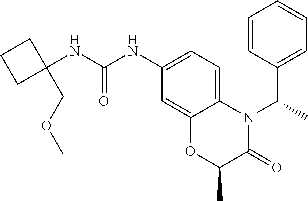 [SMILES: COCC1(NC(=O)Nc2ccc3c(c2)O[C@H](C)C(=O)N3[C@@H](C)c2ccccc2)CCC1] | 1-(1-(methoxymethyl)cyclobutyl)-3-((R)-2-methyl-3-oxo- 4-((S)-1-phenylethyl)-3,4-dihydro-2H- benzo[b][1,4]oxazin-7-yl)urea |
| | C₂₄H₂₉N₃O₄ | |
| 262 | 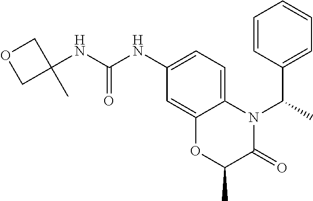 [SMILES: C[C@H]1Oc2cc(NC(=O)NC3(C)COC3)ccc2N([C@@H](C)c2ccccc2)C1=O] | 1-((R)-2-methyl-3-oxo-4-((S)-1-phenylethyl)-3,4- dihydro-2H-benzo[b][1,4]oxazin-7-yl)-3-(3- methyloxetan-3-yl)urea |
| | C₂₂H₂₅N₃O₄ | |
| 263 | 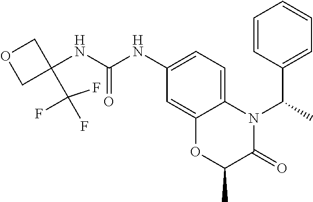 [SMILES: C[C@H]1Oc2cc(NC(=O)NC3(C(F)(F)F)COC3)ccc2N([C@@H](C)c2ccccc2)C1=O] | 1-((R)-2-methyl-3-oxo-4-((S)-1-phenylethyl)-3,4- dihydro-2H-benzo[b][1,4]oxazin-7-yl)-3-(3- (trifluoromethyl)oxetan-3-yl)urea |
| | C₂₂H₂₂F₃N₃O₄ | |
| 264 | 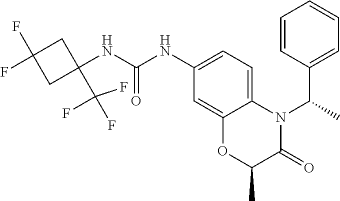 [SMILES: C[C@H]1Oc2cc(NC(=O)NC3(C(F)(F)F)CC(F)(F)C3)ccc2N([C@@H](C)c2ccccc2)C1=O] | 1-(3,3-difluoro-1-(trifluoromethyl)cyclobutyl)-3-((R)-2- methyl-3-oxo-4-((S)-1-phenylethyl)-3,4-dihydro-2H- benzo[b][1,4]oxazin-7-yl)urea |
| | C₂₃H₂₂F₅N₃O₃ | |
| 265 | 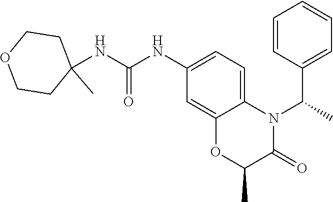 [SMILES: C[C@H]1Oc2cc(NC(=O)NC3(C)CCOCC3)ccc2N([C@@H](C)c2ccccc2)C1=O] | 1-((R)-2-methyl-3-oxo-4-((S)-1-phenylethyl)-3,4- dihydro-2H-benzo[b][1,4]oxazin-7-yl)-3-(4- methyltetrahydro-2H-pyran-4-yl)urea |
| | C₂₄H₂₉N₃O₄ | |
| 266 | 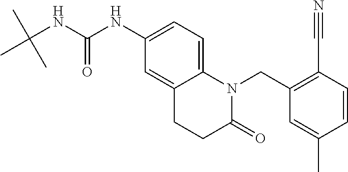 [SMILES: Cc1ccc(C#N)c(CN2C(=O)CCc3cc(NC(=O)NC(C)(C)C)ccc32)c1] | 1-(tert-butyl)-3-(1-(2-cyano-5-methylbenzyl)-2-oxo- 1,2,3,4-tetrahydroquinolin-6-yl)urea |
| | C₂₃H₂₆N₄O₂ | |
| 267 | 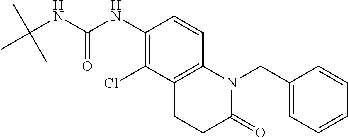 [SMILES: CC(C)(C)NC(=O)Nc1ccc2c(c1Cl)CCC(=O)N2Cc1ccccc1] | 1-(1-benzyl-5-chloro-2-oxo-1,2,3,4-tetrahydroquinolin-6- yl)-3-(tert-butyl)urea |
| | C₂₁H₂₄ClN₃O₂ | |
| 268 | 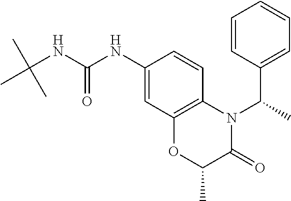 [SMILES: C[C@@H]1Oc2cc(NC(=O)NC(C)(C)C)ccc2N([C@@H](C)c2ccccc2)C1=O] | 1-(tert-butyl)-3-((S)-2-methyl-3-oxo-4-((S)-1- phenylethyl)-3,4-dihydro-2H-benzo[b][1,4]oxazin-7- yl)urea |
| | C₂₂H₂₇N₃O₃ | |
| 269 | 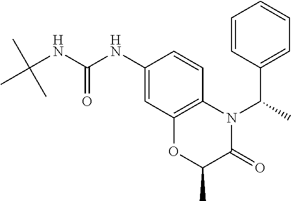 [SMILES: C[C@H]1Oc2cc(NC(=O)NC(C)(C)C)ccc2N([C@@H](C)c2ccccc2)C1=O] | 1-(tert-butyl)-3-((R)-2-methyl-3-oxo-4-((S)-1- phenylethyl)-3,4-dihydro-2H-benzo[b][1,4]oxazin-7- yl)urea |
| | C₂₂H₂₇N₃O₃ | |
| 270 | 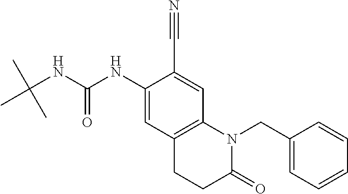 [SMILES: CC(C)(C)NC(=O)Nc1cc2c(cc1C#N)N(Cc1ccccc1)C(=O)CC2] | 1-(1-benzyl-7-cyano-2-oxo-1,2,3,4-tetrahydroquinolin-6- yl)-3-(tert-butyl)urea |
| | C₂₂H₂₄N₄O₂ | |
| 271 | 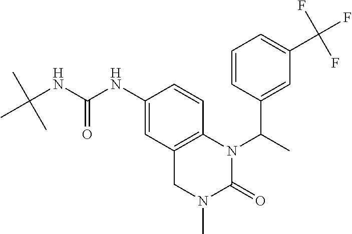 [SMILES: CC(c1cccc(C(F)(F)F)c1)N1C(=O)N(C)Cc2cc(NC(=O)NC(C)(C)C)ccc21] | 1-(tert-butyl)-3-(3-methyl-2-oxo-1-(1-(3- (trifluoromethyl)phenyl)ethyl)-1,2,3,4- tetrahydroquinazolin-6-yl)urea |
| | C₂₃H₂₇F₃N₄O₂ | |
| 272 | 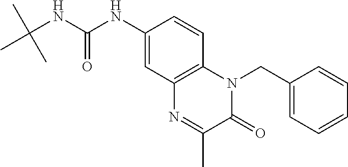 [SMILES: Cc1nc2cc(NC(=O)NC(C)(C)C)ccc2n(Cc2ccccc2)c1=O] | 1-(1-benzyl-3-methyl-2-oxo-1,2-dihydroquinoxalin-6-yl)- 3-(tert-butyl)urea |
| | C₂₁H₂₄N₄O₂ | |
| 273 | 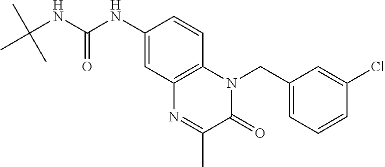 [SMILES: Cc1nc2cc(NC(=O)NC(C)(C)C)ccc2n(Cc2cccc(Cl)c2)c1=O] | 1-(tert-butyl)-3-(1-(3-chlorobenzyl)-3-methyl-2-oxo-1,2- dihydroquinoxalin-6-yl)urea |
| | C₂₁H₂₃ClN₄O₂ | |
| 274 | 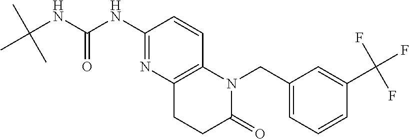 [SMILES: CC(C)(C)NC(=O)Nc1ccc2c(n1)CCC(=O)N2Cc1cccc(C(F)(F)F)c1] | 1-(tert-butyl)-3-(6-oxo-5-(3-(trifluoromethyl)benzyl)- 5,6,7,8-tetrahydro-1,5-naphthyridin-2-yl)urea |
| | C₂₁H₂₃F₃N₄O₂ | |
| 275 | 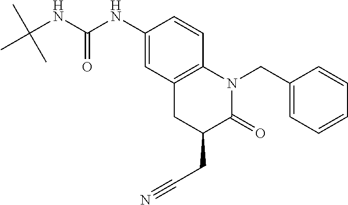 [SMILES: CC(C)(C)NC(=O)Nc1ccc2c(c1)C[C@H](CC#N)C(=O)N2Cc1ccccc1] | (R)-1-(1-benzyl-3-(cyanomethyl)-2-oxo-1,2,3,4- tetrahydroquinolin-6-yl)-3-(tert-butyl)urea |
| | C₂₃H₂₆N₄O₂ | |
| 276 | 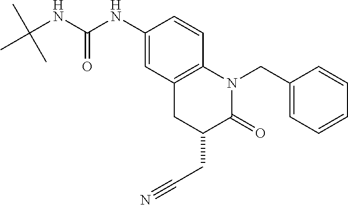 [SMILES: CC(C)(C)NC(=O)Nc1ccc2c(c1)C[C@@H](CC#N)C(=O)N2Cc1ccccc1] | (S)-1-(1-benzyl-3-(cyanomethyl)-2-oxo-1,2,3,4- tetrahydroquinolin-6-yl)-3-(tert-butyl)urea |
| | C₂₃H₂₆N₄O₂ | |
| 277 | 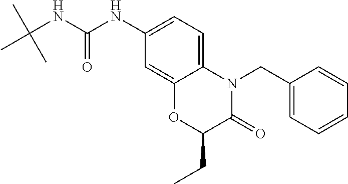 [SMILES: CC[C@H]1Oc2cc(NC(=O)NC(C)(C)C)ccc2N(Cc2ccccc2)C1=O] | (R)-1-(4-benzyl-2-ethyl-3-oxo-3,4-dihydro-2H- benzo[b][1,4]oxazin-7-yl)-3-(tert-butyl)urea |
| | C₂₂H₂₇N₃O₃ | |
| 278 | 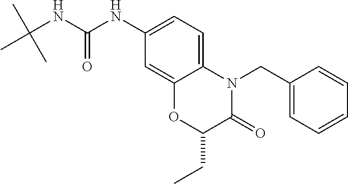 [SMILES: CC[C@@H]1Oc2cc(NC(=O)NC(C)(C)C)ccc2N(Cc2ccccc2)C1=O] | (S)-1-(4-benzyl-2-ethyl-3-oxo-3,4-dihydro-2H- benzo[b][1,4]oxazin-7-yl)-3-(tert-butyl)urea |
| | C₂₂H₂₇N₃O₃ | |
| 279 | 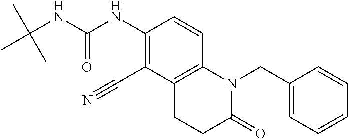 [SMILES: CC(C)(C)NC(=O)Nc1ccc2c(c1C#N)CCC(=O)N2Cc1ccccc1] | 1-(1-benzyl-5-cyano-2-oxo-1,2,3,4-tetrahydroquinolin-6- yl)-3-(tert-butyl)urea |
| | C₂₂H₂₄N₄O₂ | |
| 280 | 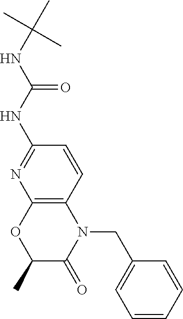 [SMILES: C[C@H]1Oc2nc(NC(=O)NC(C)(C)C)ccc2N(Cc2ccccc2)C1=O] | (R)-1-(1-benzyl-3-methyl-2-oxo-2,3-dihydro-1H- pyrido[2,3-b][1,4]oxazin-6-yl)-3-(tert-butyl)urea |
| | C₂₀H₂₄N₄O₃ | |
| 281 | 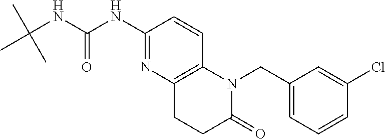 [SMILES: CC(C)(C)NC(=O)Nc1ccc2c(n1)CCC(=O)N2Cc1cccc(Cl)c1] | 1-(tert-butyl)-3-(5-(3-chlorobenzyl)-6-oxo-5,6,7,8- tetrahydro-1,5-naphthyridin-2-yl)urea |
| | C₂₀H₂₃ClN₄O₂ | |
| 282 | 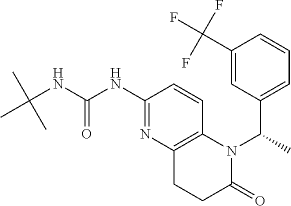 [SMILES: C[C@@H](c1cccc(C(F)(F)F)c1)N1C(=O)CCc2nc(NC(=O)NC(C)(C)C)ccc21] | (S)-1-(tert-butyl)-3-(6-oxo-5-(1-(3- (trifluoromethyl)phenyl)ethyl)-5,6,7,8-tetrahydro-1,5- naphthyridin-2-yl)urea |
| | C₂₂H₂₅F₃N₄O₂ | |
| 283 | 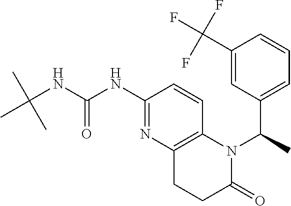 [SMILES: C[C@H](c1cccc(C(F)(F)F)c1)N1C(=O)CCc2nc(NC(=O)NC(C)(C)C)ccc21] | (R)-1-(tert-butyl)-3-(6-oxo-5-(1-(3- (trifluoromethyl)phenyl)ethyl)-5,6,7,8-tetrahydro-1,5- naphthyridin-2-yl)urea |
| | C₂₂H₂₅F₃N₄O₂ | |
| 284 | 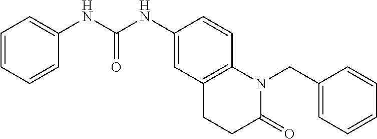 [SMILES: O=C(Nc1ccccc1)Nc1ccc2c(c1)CCC(=O)N2Cc1ccccc1] | 1-(1-benzyl-2-oxo-1,2,3,4-tetrahydroquinolin-6-yl)-3- phenylurea |
| | C₂₃H₂₁N₃O₂ | |
| 285 | 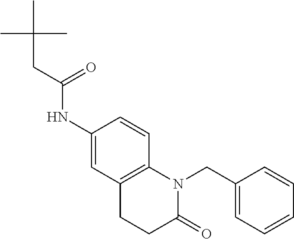 [SMILES: CC(C)(C)CC(=O)Nc1ccc2c(c1)CCC(=O)N2Cc1ccccc1] | N-(1-benzyl-2-oxo-1,2,3,4-tetrahydroquinolin-6-yl)-3,3- dimethylbutanamide |
| | C₂₂H₂₆N₂O₂ | |
| 286 | 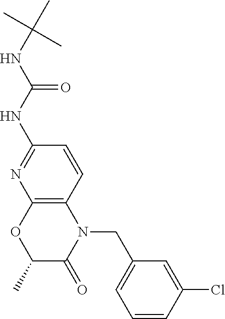 [SMILES: C[C@@H]1Oc2nc(NC(=O)NC(C)(C)C)ccc2N(Cc2cccc(Cl)c2)C1=O] | (S)-1-(tert-butyl)-3-(1-(3-chlorobenzyl)-3-methyl-2-oxo- 2,3-dihydro-1H-pyrido[2,3-b][1,4]oxazin-6-yl)urea |
| | C₂₀H₂₃ClN₄O₃ | |
| 287 | 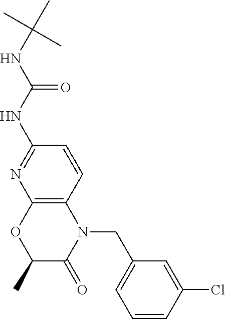 [SMILES: C[C@H]1Oc2nc(NC(=O)NC(C)(C)C)ccc2N(Cc2cccc(Cl)c2)C1=O] | (R)-1-(tert-butyl)-3-(1-(3-chlorobenzyl)-3-methyl-2-oxo- 2,3-dihydro-1H-pyrido[2,3-b][1,4]oxazin-6-yl)urea |
| | C₂₀H₂₃ClN₄O₃ | |
| 288 | 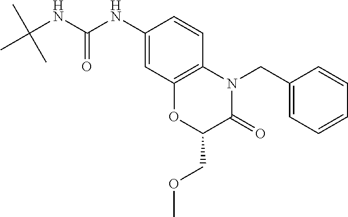 [SMILES: COC[C@@H]1Oc2cc(NC(=O)NC(C)(C)C)ccc2N(Cc2ccccc2)C1=O] | (S)-1-(4-benzyl-2-(methoxymethyl)-3-oxo-3,4-dihydro- 2H-benzo[b][1,4]oxazin-7-yl)-3-(tert-butyl)urea |
| | C₂₂H₂₇N₃O₄ | |
| 289 | 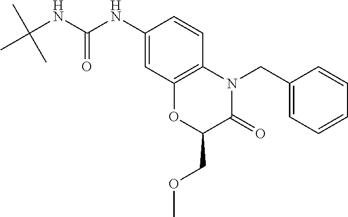 [SMILES: COC[C@H]1Oc2cc(NC(=O)NC(C)(C)C)ccc2N(Cc2ccccc2)C1=O] | (R)-1-(4-benzyl-2-(methoxymethyl)-3-oxo-3,4-dihydro- 2H-benzo[b][1,4]oxazin-7-yl)-3-(tert-butyl)urea |
| | C₂₂H₂₇N₃O₄ | |
| 290 | 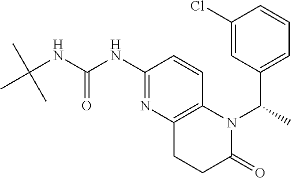 [SMILES: C[C@@H](c1cccc(Cl)c1)N1C(=O)CCc2nc(NC(=O)NC(C)(C)C)ccc21] | (S)-1-(tert-butyl)-3-(5-(1-(3-chlorophenyl)ethyl)-6-oxo- 5,6,7,8-tetrahydro-1,5-naphthyridin-2-yl)urea |
| | C₂₁H₂₅ClN₄O₂ | |
| 291 | 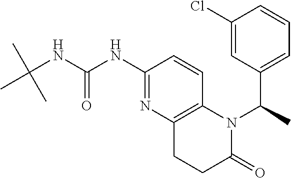 [SMILES: C[C@H](c1cccc(Cl)c1)N1C(=O)CCc2nc(NC(=O)NC(C)(C)C)ccc21] | (R)-1-(tert-butyl)-3-(5-(1-(3-chlorophenyl)ethyl)-6-oxo- 5,6,7,8-tetrahydro-1,5-naphthyridin-2-yl)urea |
| | C₂₁H₂₅ClN₄O₂ | |
| 292 | 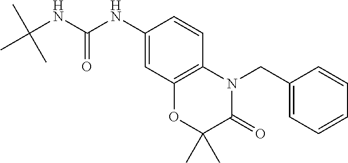 [SMILES: CC(C)(C)NC(=O)Nc1ccc2c(c1)OC(C)(C)C(=O)N2Cc1ccccc1] | 1-(4-benzyl-2,2-dimethyl-3-oxo-3,4-dihydro-2H- benzo[b][1,4]oxazin-7-yl)-3-(tert-butyl)urea |
| | C₂₂H₂₇N₃O₃ | |
| 293 | 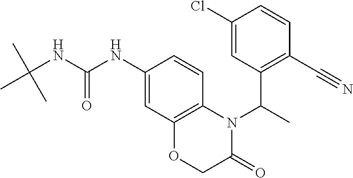 [SMILES: CC(c1cc(Cl)ccc1C#N)N1C(=O)COc2cc(NC(=O)NC(C)(C)C)ccc21] | 1-(tert-butyl)-3-(4-(1-(5-chloro-2-cyanophenyl)ethyl)-3- oxo-3,4-dihydro-2H-benzo[b][1,4]oxazin-7-yl)urea |
| | C₂₂H₂₃ClN₄O₃ | |
| 294 | 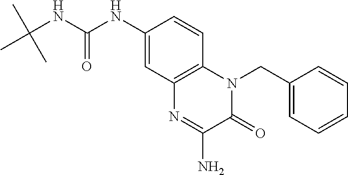 [SMILES: CC(C)(C)NC(=O)Nc1ccc2c(c1)nc(N)c(=O)n2Cc1ccccc1] | 1-(3-amino-1-benzyl-2-oxo-1,2-dihydroquinoxalin-6-yl)- 3-(tert-butyl)urea |
| | C₂₀H₂₃N₅O₂ | |
| 295 | 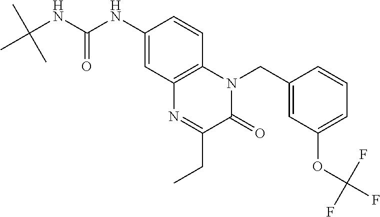 [SMILES: CCc1nc2cc(NC(=O)NC(C)(C)C)ccc2n(Cc2cccc(OC(F)(F)F)c2)c1=O] | 1-(tert-butyl)-3-(3-ethyl-2-oxo-1-(3- (trifluoromethoxy)benzyl)-1,2-dihydroquinoxalin-6- yl)urea |
| | C₂₃H₂₅F₃N₄O₃ | |
| 296 | 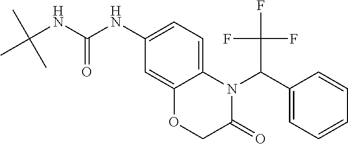 [SMILES: CC(C)(C)NC(=O)Nc1ccc2c(c1)OCC(=O)N2C(c1ccccc1)C(F)(F)F] | 1-(tert-butyl)-3-(3-oxo-4-(2,2,2-trifluoro-1-phenylethyl)- 3,4-dihydro-2H-benzo[b][1,4]oxazin-7-yl)urea |
| | C₂₁H₂₂F₃N₃O₃ | |
| 297 | 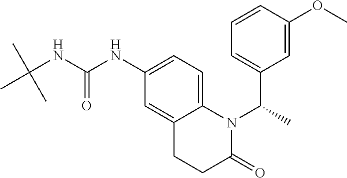 [SMILES: COc1cccc([C@H](C)N2C(=O)CCc3cc(NC(=O)NC(C)(C)C)ccc32)c1] | (S)-1-(tert-butyl)-3-(1-(1-(3-methoxyphenyl)ethyl)-2-oxo- 1,2,3,4-tetrahydroquinolin-6-yl)urea |
| | C₂₃H₂₉N₃O₃ | |
| 298 | 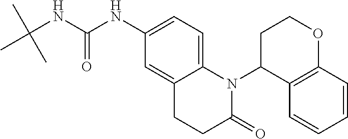 [SMILES: CC(C)(C)NC(=O)Nc1ccc2c(c1)CCC(=O)N2C1CCOc2ccccc21] | 1-(tert-butyl)-3-(1-(chroman-4-yl)-2-oxo-1,2,3,4- tetrahydroquinolin-6-yl)urea |
| | C₂₃H₂₇N₃O₃ | |
| 299 | 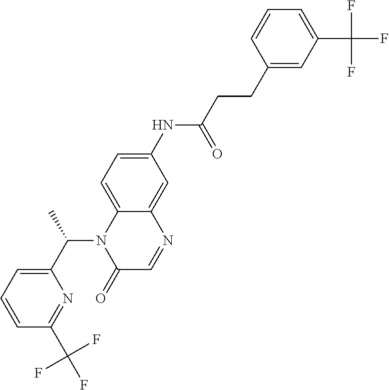 [SMILES: C[C@@H](c1cccc(C(F)(F)F)n1)n1c(=O)cnc2cc(NC(=O)CCc3cccc(C(F)(F)F)c3)ccc21] | (S)-N-(2-oxo-1-(1-(6-(trifluoromethyl)pyridin-2-yl)ethyl)- 1,2-dihydroquinoxalin-6-yl)-3-(3- (trifluoromethyl)phenyl)propanamide |
| | C₂₆H₂₀F₆N₄O₂ | |
| 300 | 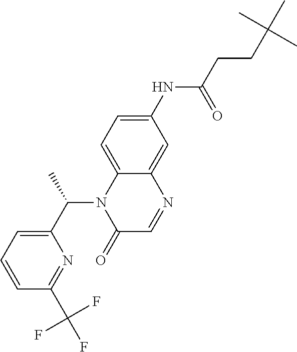 [SMILES: C[C@@H](c1cccc(C(F)(F)F)n1)n1c(=O)cnc2cc(NC(=O)CCC(C)(C)C)ccc21] | (S)-4,4-dimethyl-N-(2-oxo-1-(1-(6- (trifluoromethyl)pyridin-2-yl)ethyl)-1,2- dihydroquinoxalin-6-yl)pentanamide |
| | C₂₃H₂₅F₃N₄O₂ | |
| 301 | 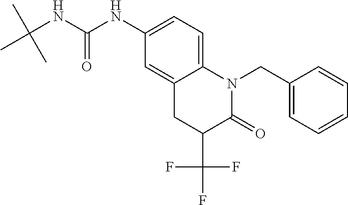 [SMILES: CC(C)(C)NC(=O)Nc1ccc2c(c1)CC(C(F)(F)F)C(=O)N2Cc1ccccc1] | 1-(1-benzyl-2-oxo-3-(trifluoromethyl)-1,2,3,4- tetrahydroquinolin-6-yl)-3-(tert-butyl)urea |
| | C₂₂H₂₄F₃N₃O₂ | |
| 302 | 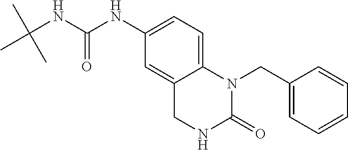 [SMILES: CC(C)(C)NC(=O)Nc1ccc2c(c1)CNC(=O)N2Cc1ccccc1] | 1-(1-benzyl-2-oxo-1,2,3,4-tetrahydroquinazolin-6-yl)-3- (tert-butyl)urea |
| | C₂₀H₂₄N₄O₂ | |
| 303 | 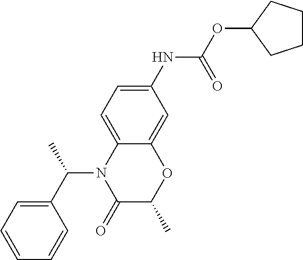 [SMILES: C[C@H]1Oc2cc(NC(=O)OC3CCCC3)ccc2N([C@@H](C)c2ccccc2)C1=O] | cyclopentyl ((R)-2-methyl-3-oxo-4-((S)-1-phenylethyl)- 3,4-dihydro-2H-benzo[b][1,4]oxazin-7-yl)carbamate |
| | C₂₃H₂₆N₂O₄ | |
| 304 | 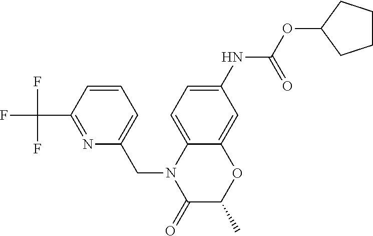 [SMILES: C[C@H]1Oc2cc(NC(=O)OC3CCCC3)ccc2N(Cc2cccc(C(F)(F)F)n2)C1=O] | cyclopentyl (R)-(2-methyl-3-oxo-4-((6- (trifluoromethyl)pyridin-2-yl)methyl)-3,4-dihydro-2H- benzo[b][1,4]oxazin-7-yl)carbamate |
| | C₂₂H₂₂F₃N₃O₄ | |
| 305 | 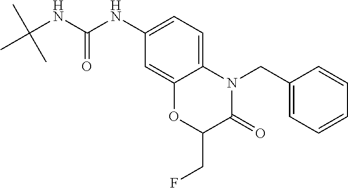 [SMILES: CC(C)(C)NC(=O)Nc1ccc2c(c1)OC(CF)C(=O)N2Cc1ccccc1] | 1-(4-benzyl-2-(fluoromethyl)-3-oxo-3,4-dihydro-2H- benzo[b][1,4]oxazin-7-yl)-3-(tert-butyl)urea |
| | C₂₁H₂₄FN₃O₃ | |
| 306 | 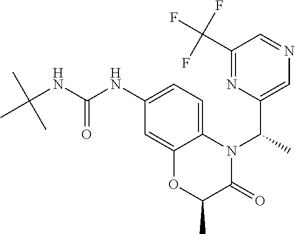 [SMILES: C[C@H]1Oc2cc(NC(=O)NC(C)(C)C)ccc2N([C@@H](C)c2cncc(C(F)(F)F)n2)C1=O] | 1-(tert-butyl)-3-((R)-2-methyl-3-oxo-4-((S)-1-(6- (trifluoromethyl)pyrazin-2-yl)ethyl)-3,4-dihydro-2H- benzo[b][1,4]oxazin-7-yl)urea |
| | C₂₁H₂₄F₃N₅O₃ | |
| 307 | 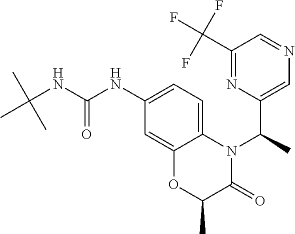 [SMILES: C[C@H](c1cncc(C(F)(F)F)n1)N1C(=O)[C@@H](C)Oc2cc(NC(=O)NC(C)(C)C)ccc21] | 1-(tert-butyl)-3-((R)-2-methyl-3-oxo-4-((R)-1-(6- (trifluoromethyl)pyrazin-2-yl)ethyl)-3,4-dihydro-2H- benzo[b][1,4]oxazin-7-yl)urea |
| | C₂₁H₂₄F₃N₅O₃ | |
| 308 | 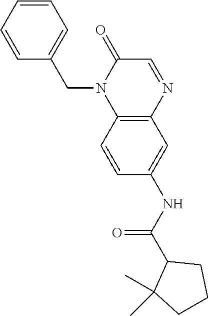 [SMILES: CC1(C)CCCC1C(=O)Nc1ccc2c(c1)ncc(=O)n2Cc1ccccc1] | N-(1-benzyl-2-oxo-1,2-dihydroquinoxalin-6-yl)-2,2- dimethylcyclopentane-1-carboxamide |
| | C₂₃H₂₅N₃O₂ | |
| 309 | 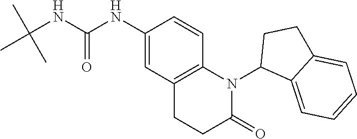 [SMILES: CC(C)(C)NC(=O)Nc1ccc2c(c1)CCC(=O)N2C1CCc2ccccc21] | 1-(tert-butyl)-3-(1-(2,3-dihydro-1H-inden-1-yl)-2-oxo- 1,2,3,4-tetrahydroquinolin-6-yl)urea |
| | C₂₃H₂₇N₃O₂ | |
| 310 | 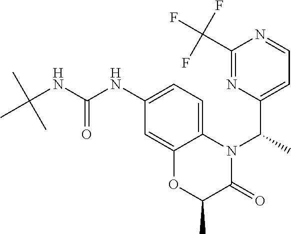 [SMILES: C[C@H]1Oc2cc(NC(=O)NC(C)(C)C)ccc2N([C@@H](C)c2ccnc(C(F)(F)F)n2)C1=O] | 1-(tert-butyl)-3-((R)-2-methyl-3-oxo-4-((S)-1-(2- (trifluoromethyl)pyrimidin-4-yl)ethyl)-3,4-dihydro-2H- benzo[b][1,4]oxazin-7-yl)urea |
| | C₂₁H₂₄F₃N₅O₃ | |
| 311 | 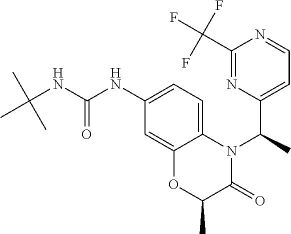 [SMILES: C[C@H](c1ccnc(C(F)(F)F)n1)N1C(=O)[C@@H](C)Oc2cc(NC(=O)NC(C)(C)C)ccc21] | 1-(tert-butyl)-3-((R)-2-methyl-3-oxo-4-((R)-1-(2- (trifluoromethyl)pyrimidin-4-yl)ethyl)-3,4-dihydro-2H- benzo[b][1,4]oxazin-7-yl)urea |
| | C₂₁H₂₄F₃N₅O₃ | |
| 312 | 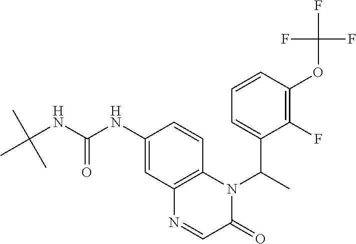 [SMILES: CC(c1cccc(OC(F)(F)F)c1F)n1c(=O)cnc2cc(NC(=O)NC(C)(C)C)ccc21] | 1-(tert-butyl)-3-(1-(1-(2-fluoro-3- (trifluoromethoxy)phenyl)ethyl)-2-oxo-1,2- dihydroquinoxalin-6-yl)urea |
| | C₂₂H₂₂F₄N₄O₃ | |
| 313 | 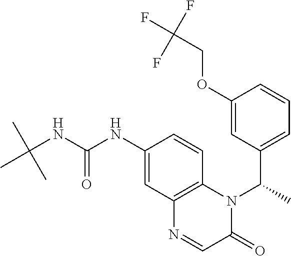 [SMILES: C[C@@H](c1cccc(OCC(F)(F)F)c1)n1c(=O)cnc2cc(NC(=O)NC(C)(C)C)ccc21] | (S)-1-(tert-butyl)-3-(2-oxo-1-(1-(3-(2,2,2- trifluoroethoxy)phenyl)ethyl)-1,2-dihydroquinoxalin-6- yl)urea |
| | C₂₃H₂₅F₃N₄O₃ | |
| 314 | 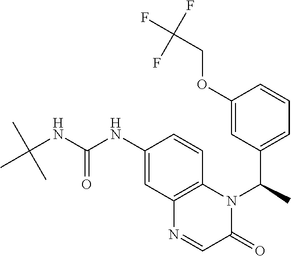 [SMILES: C[C@H](c1cccc(OCC(F)(F)F)c1)n1c(=O)cnc2cc(NC(=O)NC(C)(C)C)ccc21] | (R)-1-(tert-butyl)-3-(2-oxo-1-(1-(3-(2,2,2- trifluoroethoxy)phenyl)ethyl)-1,2-dihydroquinoxalin-6- yl)urea |
| | C₂₃H₂₅F₃N₄O₃ | |
| 315 | 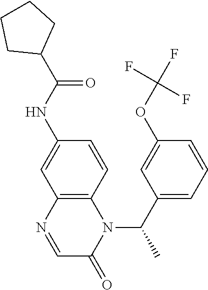 [SMILES: C[C@@H](c1cccc(OC(F)(F)F)c1)n1c(=O)cnc2cc(NC(=O)C3CCCC3)ccc21] | (S)-N-(2-oxo-1-(1-(3-(trifluoromethoxy)phenyl)ethyl)- 1,2-dihydroquinoxalin-6-yl)cyclopentanecarboxamide |
| | C₂₃H₂₂F₃N₃O₃ | |
| 316 | 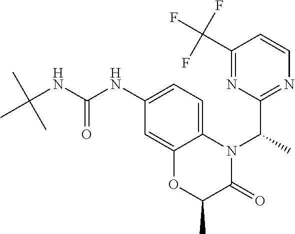 [SMILES: C[C@H]1Oc2cc(NC(=O)NC(C)(C)C)ccc2N([C@@H](C)c2nccc(C(F)(F)F)n2)C1=O] | 1-(tert-butyl)-3-((R)-2-methyl-3-oxo-4-((S)-1-(4- (trifluoromethyl)pyrimidin-2-yl)ethyl)-3,4-dihydro-2H- benzo[b][1,4]oxazin-7-yl)urea |
| | C₂₁H₂₄F₃N₅O₃ | |
| 317 | 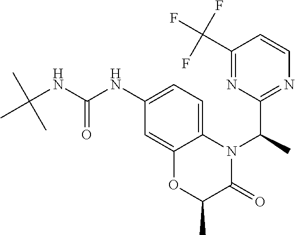 [SMILES: C[C@H](c1nccc(C(F)(F)F)n1)N1C(=O)[C@@H](C)Oc2cc(NC(=O)NC(C)(C)C)ccc21] | 1-(tert-butyl)-3-((R)-2-methyl-3-oxo-4-((R)-1-(4- (trifluoromethyl)pyrimidin-2-yl)ethyl)-3,4-dihydro-2H- benzo[b][1,4]oxazin-7-yl)urea |
| | C₂₁H₂₄F₃N₅O₃ | |
| 318 | 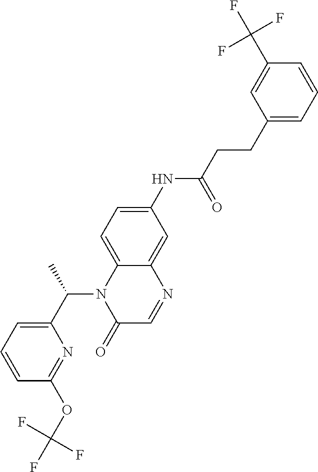 [SMILES: C[C@@H](c1cccc(OC(F)(F)F)n1)n1c(=O)cnc2cc(NC(=O)CCc3cccc(C(F)(F)F)c3)ccc21] | (S)-N-(2-oxo-1-(1-(6-(trifluoromethoxy)pyridin-2- yl)ethyl)-1,2-dihydroquinoxalin-6-yl)-3-(3- (trifluoromethyl)phenyl)propanamide |
| | C₂₆H₂₀F₆N₄O₃ | |
| 319 | 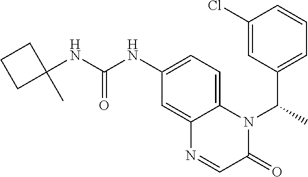 [SMILES: C[C@@H](c1cccc(Cl)c1)n1c(=O)cnc2cc(NC(=O)NC3(C)CCC3)ccc21] | (S)-1-(1-(1-(3-chlorophenyl)ethyl)-2-oxo-1,2- dihydroquinoxalin-6-yl)-3-(1-methylcyclobutyl)urea |
| | C₂₂H₂₃ClN₄O₂ | |
| 320 | 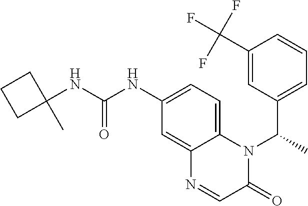 [SMILES: C[C@@H](c1cccc(C(F)(F)F)c1)n1c(=O)cnc2cc(NC(=O)NC3(C)CCC3)ccc21] | (S)-1-(1-methylcyclobutyl)-3-(2-oxo-1-(1-(3- (trifluoromethyl)phenyl)ethyl)-1,2-dihydroquinoxalin-6- yl)urea |
| | C₂₃H₂₃F₃N₄O₂ | |
| 321 | 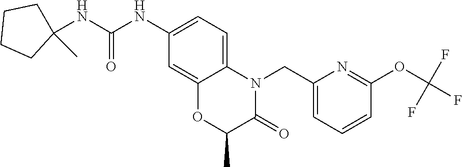 [SMILES: C[C@H]1Oc2cc(NC(=O)NC3(C)CCCC3)ccc2N(Cc2cccc(OC(F)(F)F)n2)C1=O] | (R)-1-(2-methyl-3-oxo-4-((6-(trifluoromethoxy)pyridin-2- yl)methyl)-3,4-dihydro-2H-benzo[b][1,4]oxazin-7-yl)-3- (1-methylcyclopentyl)urea |
| | C₂₃H₂₅F₃N₄O₄ | |
| 322 | 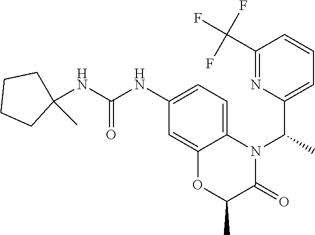 [SMILES: C[C@H]1Oc2cc(NC(=O)NC3(C)CCCC3)ccc2N([C@@H](C)c2cccc(C(F)(F)F)n2)C1=O] | 1-((R)-2-methyl-3-oxo-4-((S)-1-(6- (trifluoromethyl)pyridin-2-yl)ethyl)-3,4-dihydro-2H- benzo[b][1,4]oxazin-7-yl)-3-(1-methylcyclopentyl)urea |
| | C₂₄H₂₇F₃N₄O₃ | |
| 323 | 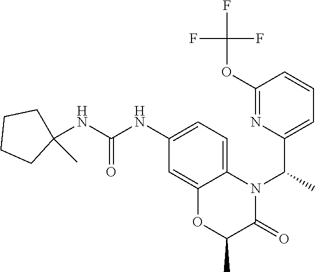 [SMILES: C[C@H]1Oc2cc(NC(=O)NC3(C)CCCC3)ccc2N([C@@H](C)c2cccc(OC(F)(F)F)n2)C1=O] | 1-((R)-2-methyl-3-oxo-4-((S)-1-(6- (trifluoromethoxy)pyridin-2-yl)ethyl)-3,4-dihydro-2H- benzo[b][1,4]oxazin-7-yl)-3-(1-methylcyclopentyl)urea |
| | C₂₄H₂₇F₃N₄O₄ | |
| 324 | 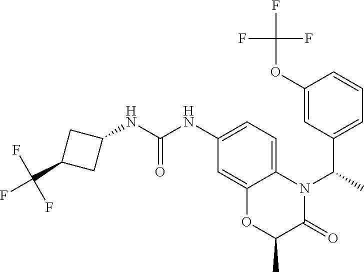 [SMILES: C[C@H]1Oc2cc(NC(=O)N[C@H]3C[C@H](C(F)(F)F)C3)ccc2N([C@@H](C)c2cccc(OC(F)(F)F)c2)C1=O] | 1-((R)-2-methyl-3-oxo-4-((S)-1-(3- (trifluoromethoxy)phenyl)ethyl)-3,4-dihydro-2H- benzo[b][1,4]oxazin-7-yl)-3-((1r,3R)-3- (trifluoromethyl)cyclobutyl)urea |
| | C₂₄H₂₃F₆N₃O₄ | |
| 325 | 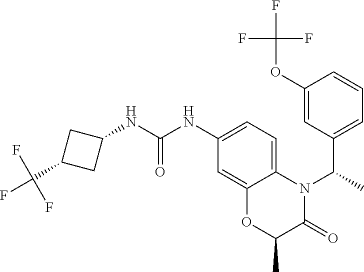 [SMILES: C[C@H]1Oc2cc(NC(=O)N[C@H]3C[C@@H](C(F)(F)F)C3)ccc2N([C@@H](C)c2cccc(OC(F)(F)F)c2)C1=O] | 1-((R)-2-methyl-3-oxo-4-((S)-1-(3- (trifluoromethoxy)phenyl)ethyl)-3,4-dihydro-2H- benzo[b][1,4]oxazin-7-yl)-3-((1s,3S)-3- (trifluoromethyl)cyclobutyl)urea |
| | C₂₄H₂₃F₆N₃O₄ | |
| 326 | 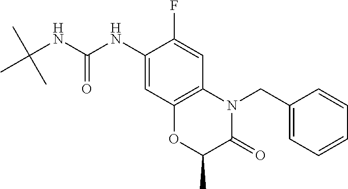 [SMILES: C[C@H]1Oc2cc(NC(=O)NC(C)(C)C)c(F)cc2N(Cc2ccccc2)C1=O] | (R)-1-(4-benzyl-6-fluoro-2-methyl-3-oxo-3,4-dihydro- 2H-benzo[b][1,4]oxazin-7-yl)-3-(tert-butyl)urea |
| | C₂₁H₂₄FN₃O₃ | |
| 327 | 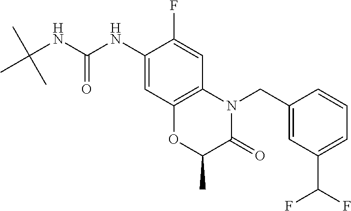 [SMILES: C[C@H]1Oc2cc(NC(=O)NC(C)(C)C)c(F)cc2N(Cc2cccc(C(F)F)c2)C1=O] | (R)-1-(tert-butyl)-3-(4-(3-(difluoromethyl)benzyl)-6- fluoro-2-methyl-3-oxo-3,4-dihydro-2H- benzo[b][1,4]oxazin-7-yl)urea |
| | C₂₂H₂₄F₃N₃O₃ | |
| 328 | 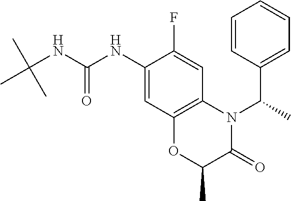 [SMILES: C[C@H]1Oc2cc(NC(=O)NC(C)(C)C)c(F)cc2N([C@@H](C)c2ccccc2)C1=O] | 1-(tert-butyl)-3-((R)-6-fluoro-2-methyl-3-oxo-4-((S)-1- phenylethyl)-3,4-dihydro-2H-benzo[b][1,4]oxazin-7- yl)urea |
| | C₂₂H₂₆FN₃O₃ | |
| 329 | 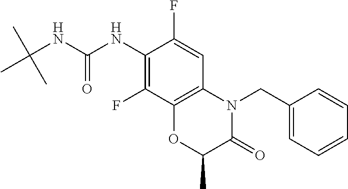 [SMILES: C[C@H]1Oc2c(cc(F)c(NC(=O)NC(C)(C)C)c2F)N(Cc2ccccc2)C1=O] | (R)-1-(4-benzyl-6,8-difluoro-2-methyl-3-oxo-3,4- dihydro-2H-benzo[b][1,4]oxazin-7-yl)-3-(tert-butyl)urea |
| | C₂₁H₂₃F₂N₃O₃ | |
| 330 | 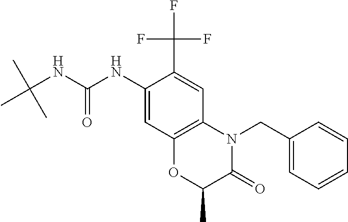 [SMILES: C[C@H]1Oc2cc(NC(=O)NC(C)(C)C)c(C(F)(F)F)cc2N(Cc2ccccc2)C1=O] | (R)-1-(4-benzyl-2-methyl-3-oxo-6-(trifluoromethyl)-3,4- dihydro-2H-benzo[b][1,4]oxazin-7-yl)-3-(tert-butyl)urea |
| | C₂₂H₂₄F₃N₃O₃ | |
| 331 | 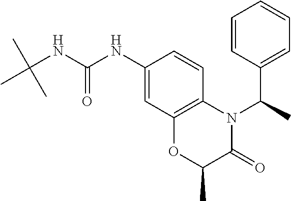 [SMILES: C[C@H](c1ccccc1)N1C(=O)[C@@H](C)Oc2cc(NC(=O)NC(C)(C)C)ccc21] | 1-(tert-butyl)-3-((R)-2-methyl-3-oxo-4-((R)-1- phenylethyl)-3,4-dihydro-2H-benzo[b][1,4]oxazin-7- yl)urea |
| | C₂₂H₂₇N₃O₃ | |
| 332 | 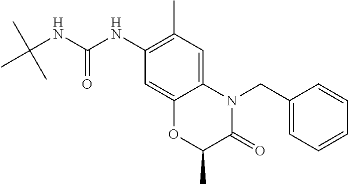 [SMILES: Cc1cc2c(cc1NC(=O)NC(C)(C)C)O[C@H](C)C(=O)N2Cc1ccccc1] | (R)-1-(4-benzyl-2,6-dimethyl-3-oxo-3,4-dihydro-2H- benzo[b][1,4]oxazin-7-yl)-3-(tert-butyl)urea |
| | C₂₂H₂₇N₃O₃ | |
| 333 | 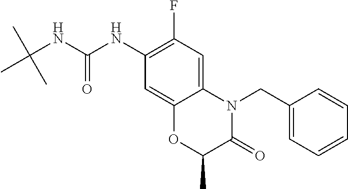 [SMILES: C[C@H]1Oc2cc(NC(=O)NC(C)(C)C)c(F)cc2N(Cc2ccccc2)C1=O] | (S)-1-(1-benzyl-7-fluoro-3-methyl-2-oxo-1,2,3,4- tetrahydroquinolin-6-yl)-3-(tert-butyl)urea |
| | C₂₂H₂₆FN₃O₂ | |
| 334 | 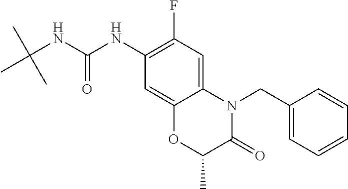 [SMILES: C[C@@H]1Oc2cc(NC(=O)NC(C)(C)C)c(F)cc2N(Cc2ccccc2)C1=O] | (R)-1-(1-benzyl-7-fluoro-3-methyl-2-oxo-1,2,3,4- tetrahydroquinolin-6-yl)-3-(tert-butyl)urea |
| | C₂₂H₂₆FN₃O₂ | |
| 335 | 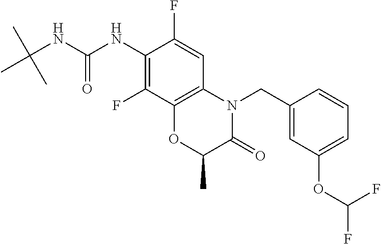 [SMILES: C[C@H]1Oc2c(cc(F)c(NC(=O)NC(C)(C)C)c2F)N(Cc2cccc(OC(F)F)c2)C1=O] | (R)-1-(tert-butyl)-3-(4-(3-(difluoromethoxy)benzyl)-6,8- difluoro-2-methyl-3-oxo-3,4-dihydro-2H- benzo[b][1,4]oxazin-7-yl)urea |
| | C₂₂H₂₃F₄N₃O₄ | |
| 336 | 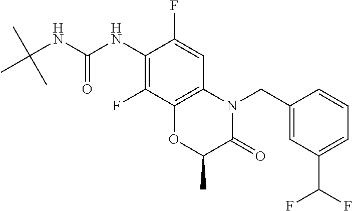 [SMILES: C[C@H]1Oc2c(cc(F)c(NC(=O)NC(C)(C)C)c2F)N(Cc2cccc(C(F)F)c2)C1=O] | (R)-1-(tert-butyl)-3-(4-(3-(difluoromethyl)benzyl)-6,8- difluoro-2-methyl-3-oxo-3,4-dihydro-2H- benzo[b][1,4]oxazin-7-yl)urea |
| | C₂₂H₂₃F₄N₃O₃ | |
| 337 | 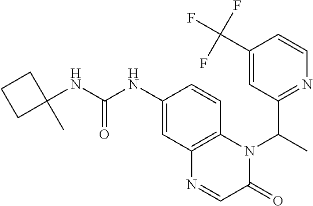 [SMILES: CC(c1cc(C(F)(F)F)ccn1)n1c(=O)cnc2cc(NC(=O)NC3(C)CCC3)ccc21] | 1-(1-methylcyclobutyl)-3-(2-oxo-1-(1-(4- (trifluoromethyl)pyridin-2-yl)ethyl)-1,2- dihydroquinoxalin-6-yl)urea |
| | C₂₂H₂₂F₃N₅O₂ | |
| 338 | 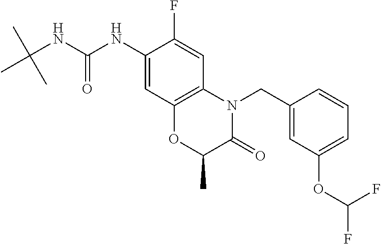 [SMILES: C[C@H]1Oc2cc(NC(=O)NC(C)(C)C)c(F)cc2N(Cc2cccc(OC(F)F)c2)C1=O] | (R)-1-(tert-butyl)-3-(4-(3-(difluoromethoxy)benzyl)-6- fluoro-2-methyl-3-oxo-3,4-dihydro-2H- benzo[b][1,4]oxazin-7-yl)urea |
| | C₂₂H₂₄F₃N₃O₄ | |
| 339 | 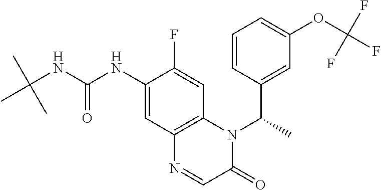 [SMILES: C[C@@H](c1cccc(OC(F)(F)F)c1)n1c(=O)cnc2cc(NC(=O)NC(C)(C)C)c(F)cc21] | (S)-1-(tert-butyl)-3-(7-fluoro-2-oxo-1-(1-(3- (trifluoromethoxy)phenyl)ethyl)-1,2-dihydroquinoxalin-6- yl)urea |
| | C₂₂H₂₂F₄N₄O₃ | |
| 340 | 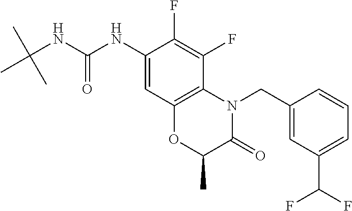 [SMILES: C[C@H]1Oc2cc(NC(=O)NC(C)(C)C)c(F)c(F)c2N(Cc2cccc(C(F)F)c2)C1=O] | (R)-1-(tert-butyl)-3-(4-(3-(difluoromethyl)benzyl)-5,6- difluoro-2-methyl-3-oxo-3,4-dihydro-2H- benzo[b][1,4]oxazin-7-yl)urea |
| | C₂₂H₂₃F₄N₃O₃ | |
| 341 | 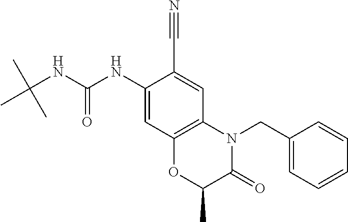 [SMILES: C[C@H]1Oc2cc(NC(=O)NC(C)(C)C)c(C#N)cc2N(Cc2ccccc2)C1=O] | (R)-1-(4-benzyl-6-cyano-2-methyl-3-oxo-3,4-dihydro- 2H-benzo[b][1,4]oxazin-7-yl)-3-(tert-butyl)urea |
| | C₂₂H₂₄N₄O₃ | |
| 342 | 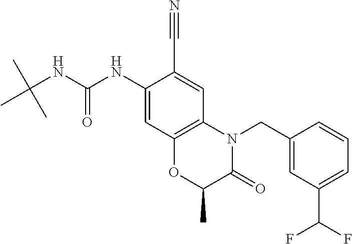 [SMILES: C[C@H]1Oc2cc(NC(=O)NC(C)(C)C)c(C#N)cc2N(Cc2cccc(C(F)F)c2)C1=O] | (R)-1-(tert-butyl)-3-(6-cyano-4-(3- (difluoromethyl)benzyl)-2-methyl-3-oxo-3,4-dihydro-2H- benzo[b][1,4]oxazin-7-yl)urea |
| | C₂₃H₂₄F₂N₄O₃ | |
| 343 | 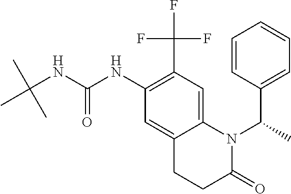 [SMILES: C[C@@H](c1ccccc1)N1C(=O)CCc2cc(NC(=O)NC(C)(C)C)c(C(F)(F)F)cc21] | (S)-1-(tert-butyl)-3-(2-oxo-1-(1-phenylethyl)-7- (trifluoromethyl)-1,2,3,4-tetrahydroquinolin-6-yl)urea |
| | C₂₃H₂₆F₃N₃O₂ | |
| 344 | 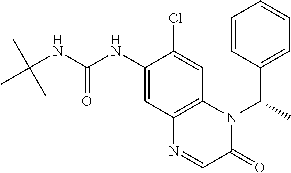 [SMILES: C[C@@H](c1ccccc1)n1c(=O)cnc2cc(NC(=O)NC(C)(C)C)c(Cl)cc21] | (S)-1-(tert-butyl)-3-(7-chloro-2-oxo-1-(1-phenylethyl)- 1,2-dihydroquinoxalin-6-yl)urea |
| | C₂₁H₂₃ClN₄O₂ | |
| 345 | 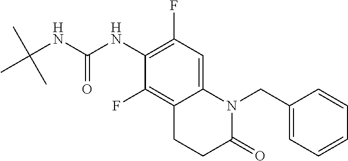 [SMILES: CC(C)(C)NC(=O)Nc1c(F)cc2c(c1F)CCC(=O)N2Cc1ccccc1] | 1-(1-benzyl-5,7-difluoro-2-oxo-1,2,3,4- tetrahydroquinolin-6-yl)-3-(tert-butyl)urea |
| | C₂₁H₂₃F₂N₃O₂ | |
| 346 | 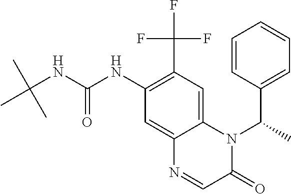 [SMILES: C[C@@H](c1ccccc1)n1c(=O)cnc2cc(NC(=O)NC(C)(C)C)c(C(F)(F)F)cc21] | (S)-1-(tert-butyl)-3-(2-oxo-1-(1-phenylethyl)-7- (trifluoromethyl)-1,2-dihydroquinoxalin-6-yl)urea |
| | C₂₂H₂₃F₃N₄O₂ | |
| 347 | 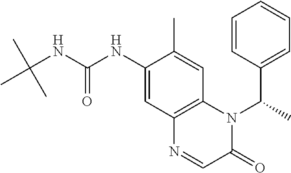 [SMILES: Cc1cc2c(cc1NC(=O)NC(C)(C)C)ncc(=O)n2[C@@H](C)c1ccccc1] | (S)-1-(tert-butyl)-3-(7-methyl-2-oxo-1-(1-phenylethyl)- 1,2-dihydroquinoxalin-6-yl)urea |
| | C₂₂H₂₆N₄O₂ | |
| 348 | 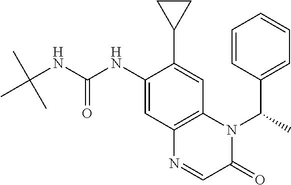 [SMILES: C[C@@H](c1ccccc1)n1c(=O)cnc2cc(NC(=O)NC(C)(C)C)c(C3CC3)cc21] | (S)-1-(tert-butyl)-3-(7-cyclopropyl-2-oxo-1-(1- phenylethyl)-1,2-dihydroquinoxalin-6-yl)urea |
| | C₂₄H₂₈N₄O₂ | |
| 349 | 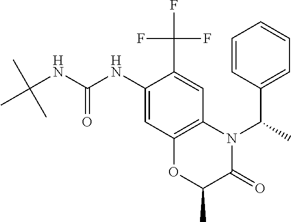 [SMILES: C[C@H]1Oc2cc(NC(=O)NC(C)(C)C)c(C(F)(F)F)cc2N([C@@H](C)c2ccccc2)C1=O] | 1-(tert-butyl)-3-((R)-2-methyl-3-oxo-4-((S)-1- phenylethyl)-6-(trifluoromethyl)-3,4-dihydro-2H- benzo[b][1,4]oxazin-7-yl)urea |
| | C₂₃H₂₆F₃N₃O₃ | |
| 350 | 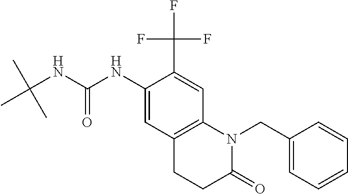 [SMILES: CC(C)(C)NC(=O)Nc1cc2c(cc1C(F)(F)F)N(Cc1ccccc1)C(=O)CC2] | 1-(1-benzyl-2-oxo-7-(trifluoromethyl)-1,2,3,4- tetrahydroquinolin-6-yl)-3-(tert-butyl)urea |
| | C₂₂H₂₄F₃N₃O₂ | |
| 351 | 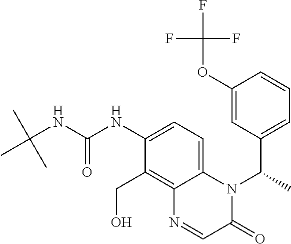 [SMILES: C[C@@H](c1cccc(OC(F)(F)F)c1)n1c(=O)cnc2c(CO)c(NC(=O)NC(C)(C)C)ccc21] | (S)-1-(tert-butyl)-3-(5-(hydroxymethyl)-2-oxo-1-(1-(3- (trifluoromethoxy)phenyl)ethyl)-1,2-dihydroquinoxalin-6- yl)urea |
| | C₂₃H₂₅F₃N₄O₄ | |
| 352 | 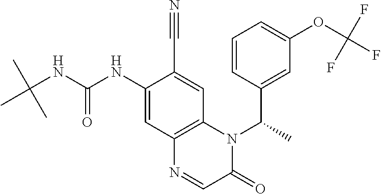 [SMILES: C[C@@H](c1cccc(OC(F)(F)F)c1)n1c(=O)cnc2cc(NC(=O)NC(C)(C)C)c(C#N)cc21] | (S)-1-(tert-butyl)-3-(7-cyano-2-oxo-1-(1-(3- (trifluoromethoxy)phenyl)ethyl)-1,2-dihydroquinoxalin-6- yl)urea |
| | C₂₃H₂₂F₃N₅O₃ | |
| 353 | 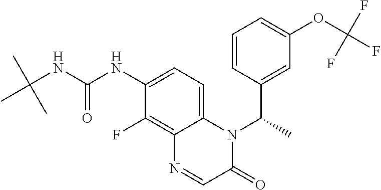 [SMILES: C[C@@H](c1cccc(OC(F)(F)F)c1)n1c(=O)cnc2c(F)c(NC(=O)NC(C)(C)C)ccc21] | (S)-1-(tert-butyl)-3-(5-fluoro-2-oxo-1-(1-(3- (trifluoromethoxy)phenyl)ethyl)-1,2-dihydroquinoxalin-6- yl)urea |
| | C₂₂H₂₂F₄N₄O₃ | |
| 354 | 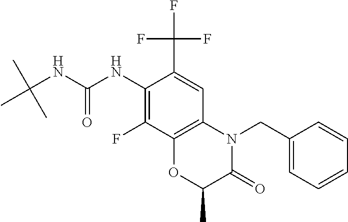 [SMILES: C[C@H]1Oc2c(cc(C(F)(F)F)c(NC(=O)NC(C)(C)C)c2F)N(Cc2ccccc2)C1=O] | (R)-1-(4-benzyl-8-fluoro-2-methyl-3-oxo-6- (trifluoromethyl)-3,4-dihydro-2H-benzo[b][1,4]oxazin-7- yl)-3-(tert-butyl)urea |
| | C₂₂H₂₃F₄N₃O₃ | |
| 355 | 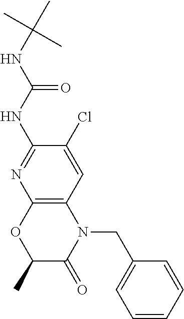 [SMILES: C[C@H]1Oc2nc(NC(=O)NC(C)(C)C)c(Cl)cc2N(Cc2ccccc2)C1=O] | (R)-1-(1-benzyl-7-chloro-3-methyl-2-oxo-2,3-dihydro- 1H-pyrido[2,3-b][1,4]oxazin-6-yl)-3-(tert-butyl)urea |
| | C₂₀H₂₃ClN₄O₃ | |
| 356 | 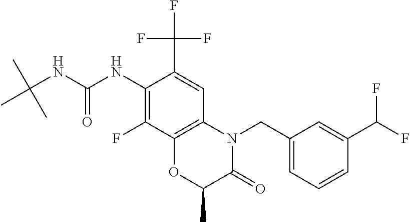 [SMILES: C[C@H]1Oc2c(cc(C(F)(F)F)c(NC(=O)NC(C)(C)C)c2F)N(Cc2cccc(C(F)F)c2)C1=O] | (R)-1-(tert-butyl)-3-(4-(3-(difluoromethyl)benzyl)-8- fluoro-2-methyl-3-oxo-6-(trifluoromethyl)-3,4-dihydro- 2H-benzo[b][1,4]oxazin-7-yl)urea |
| | C₂₃H₂₃F₆N₃O₃ | |
| 357 | 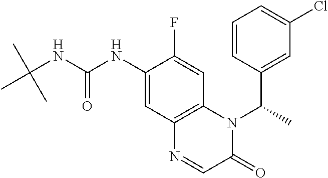 [SMILES: C[C@@H](c1cccc(Cl)c1)n1c(=O)cnc2cc(NC(=O)NC(C)(C)C)c(F)cc21] | (S)-1-(tert-butyl)-3-(1-(1-(3-chlorophenyl)ethyl)-7-fluoro- 2-oxo-1,2-dihydroquinoxalin-6-yl)urea |
| | C₂₁H₂₂ClFN₄O₂ | |
| 358 | 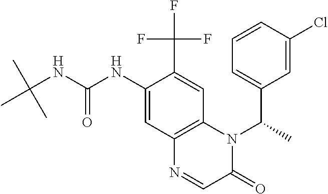 [SMILES: C[C@@H](c1cccc(Cl)c1)n1c(=O)cnc2cc(NC(=O)NC(C)(C)C)c(C(F)(F)F)cc21] | (S)-1-(tert-butyl)-3-(1-(1-(3-chlorophenyl)ethyl)-2-oxo-7- (trifluoromethyl)-1,2-dihydroquinoxalin-6-yl)urea |
| | C₂₂H₂₂ClF₃N₄O₂ | |
| 359 | 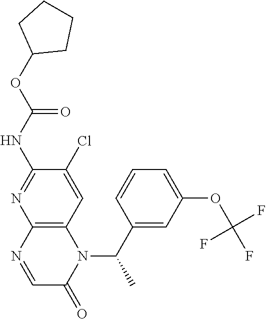 [SMILES: C[C@@H](c1cccc(OC(F)(F)F)c1)n1c(=O)cnc2nc(NC(=O)OC3CCCC3)c(Cl)cc21] | cyclopentyl (S)-(2-oxo-1-(1-(3- (trifluoromethoxy)phenyl)ethyl)-1,2-dihydroquinoxalin-6- yl)carbamate |
| | C₂₃H₂₂F₃N₃O₄ | |
| 360 | 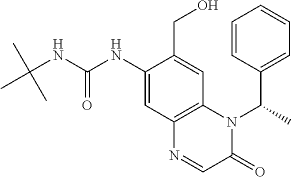 [SMILES: C[C@@H](c1ccccc1)n1c(=O)cnc2cc(NC(=O)NC(C)(C)C)c(CO)cc21] | (S)-1-(tert-butyl)-3-(7-(hydroxymethyl)-2-oxo-1-(1- phenylethyl)-1,2-dihydroquinoxalin-6-yl)urea |
| | C₂₂H₂₆N₄O₃ | |
| 361 | 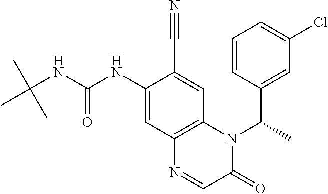 [SMILES: C[C@@H](c1cccc(Cl)c1)n1c(=O)cnc2cc(NC(=O)NC(C)(C)C)c(C#N)cc21] | (S)-1-(tert-butyl)-3-(1-(1-(3-chlorophenyl)ethyl)-7-cyano- 2-oxo-1,2-dihydroquinoxalin-6-yl)urea |
| | C₂₂H₂₂ClN₅O₂ | |
| 362 | 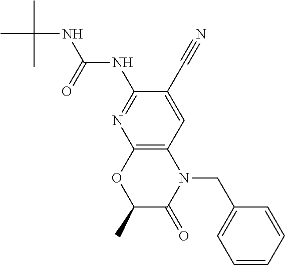 [SMILES: C[C@H]1Oc2nc(NC(=O)NC(C)(C)C)c(C#N)cc2N(Cc2ccccc2)C1=O] | (R)-1-(1-benzyl-7-cyano-3-methyl-2-oxo-2,3-dihydro- 1H-pyrido[2,3-b][1,4]oxazin-6-yl)-3-(tert-butyl)urea |
| | C₂₁H₂₃N₅O₃ | |
| 363 | 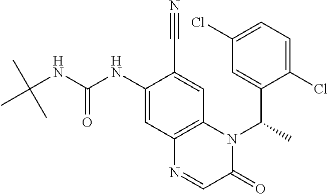 [SMILES: C[C@@H](c1cc(Cl)ccc1Cl)n1c(=O)cnc2cc(NC(=O)NC(C)(C)C)c(C#N)cc21] | (S)-1-(tert-butyl)-3-(7-cyano-1-(1-(2,5- dichlorophenyl)ethyl)-2-oxo-1,2-dihydroquinoxalin-6- yl)urea |
| | C₂₂H₂₁Cl₂N₅O₂ | |
| 364 | 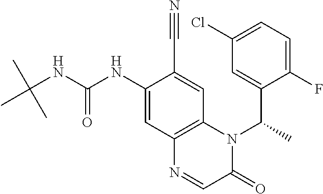 [SMILES: C[C@@H](c1cc(Cl)ccc1F)n1c(=O)cnc2cc(NC(=O)NC(C)(C)C)c(C#N)cc21] | (S)-1-(tert-butyl)-3-(1-(1-(5-chloro-2-fluorophenyl)ethyl)- 7-cyano-2-oxo-1,2-dihydroquinoxalin-6-yl)urea |
| | C₂₂H₂₁ClFN₅O₂ | |
| 365 | 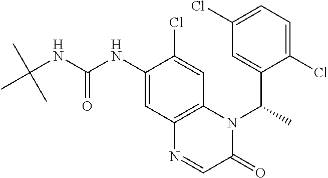 [SMILES: C[C@@H](c1cc(Cl)ccc1Cl)n1c(=O)cnc2cc(NC(=O)NC(C)(C)C)c(Cl)cc21] | (S)-1-(tert-butyl)-3-(7-chloro-1-(1-(2,5- dichlorophenyl)ethyl)-2-oxo-1,2-dihydroquinoxalin-6- yl)urea |
| | C₂₁H₂₁Cl₃N₄O₂ | |
| 366 | 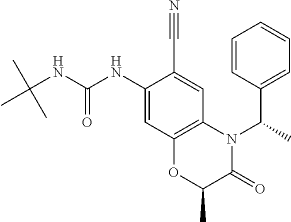 [SMILES: C[C@H]1Oc2cc(NC(=O)NC(C)(C)C)c(C#N)cc2N([C@@H](C)c2ccccc2)C1=O] | 1-(tert-butyl)-3-((R)-6-cyano-2-methyl-3-oxo-4-((S)-1- phenylethyl)-3,4-dihydro-2H-benzo[b][1,4]oxazin-7- yl)urea |
| | C₂₃H₂₆N₄O₃ | |
| 367 | 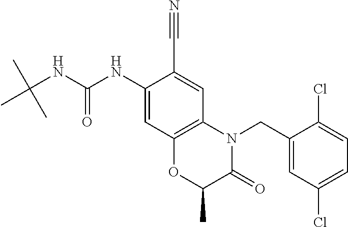 [SMILES: C[C@H]1Oc2cc(NC(=O)NC(C)(C)C)c(C#N)cc2N(Cc2cc(Cl)ccc2Cl)C1=O] | (R)-1-(tert-butyl)-3-(6-cyano-4-(2,5-dichlorobenzyl)-2- methyl-3-oxo-3,4-dihydro-2H-benzo[b][1,4]oxazin-7- yl)urea |
| | C₂₂H₂₂Cl₂N₄O₃ | |
| 368 | 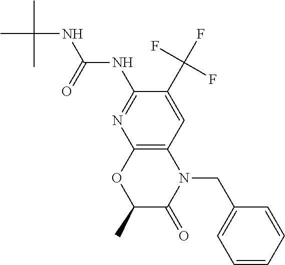 [SMILES: C[C@H]1Oc2nc(NC(=O)NC(C)(C)C)c(C(F)(F)F)cc2N(Cc2ccccc2)C1=O] | (R)-1-(1-benzyl-3-methyl-2-oxo-7-(trifluoromethyl)-2,3- dihydro-1H-pyrido[2,3-b][1,4]oxazin-6-yl)-3-(tert- butyl)urea |
| | C₂₁H₂₃F₃N₄O₃ | |
| 369 | 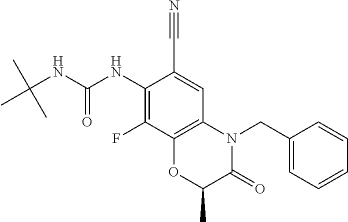 [SMILES: C[C@H]1Oc2c(cc(C#N)c(NC(=O)NC(C)(C)C)c2F)N(Cc2ccccc2)C1=O] | (R)-1-(4-benzyl-6-cyano-8-fluoro-2-methyl-3-oxo-3,4- dihydro-2H-benzo[b][1,4]oxazin-7-yl)-3-(tert-butyl)urea |
| | C₂₂H₂₃FN₄O₃ | |
| 370 | 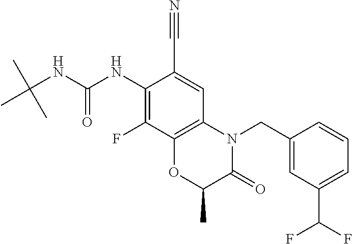 [SMILES: C[C@H]1Oc2c(cc(C#N)c(NC(=O)NC(C)(C)C)c2F)N(Cc2cccc(C(F)F)c2)C1=O] | (R)-1-(tert-butyl)-3-(6-cyano-4-(3- (difluoromethyl)benzyl)-8-fluoro-2-methyl-3-oxo-3,4- dihydro-2H-benzo[b][1,4]oxazin-7-yl)urea |
| | C₂₃H₂₃F₃N₄O₃ | |
| 371 | 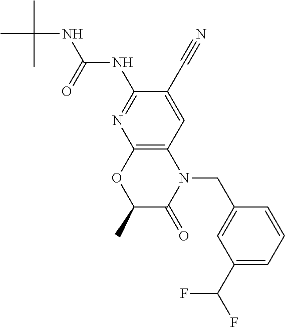 [SMILES: C[C@H]1Oc2nc(NC(=O)NC(C)(C)C)c(C#N)cc2N(Cc2cccc(C(F)F)c2)C1=O] | (R)-1-(tert-butyl)-3-(7-cyano-1-(3- (difluoromethyl)benzyl)-3-methyl-2-oxo-2,3-dihydro-1H- pyrido[2,3-b][1,4]oxazin-6-yl)urea |
| | C₂₂H₂₃F₂N₅O₃ | |
| 372 | 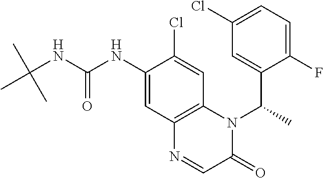 [SMILES: C[C@@H](c1cc(Cl)ccc1F)n1c(=O)cnc2cc(NC(=O)NC(C)(C)C)c(Cl)cc21] | (S)-1-(tert-butyl)-3-(7-chloro-1-(1-(5-chloro-2- fluorophenyl)ethyl)-2-oxo-1,2-dihydroquinoxalin-6- yl)urea |
| | C₂₁H₂₁Cl₂FN₄O₂ | |
| 373 | 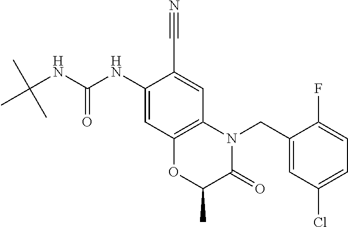 [SMILES: C[C@H]1Oc2cc(NC(=O)NC(C)(C)C)c(C#N)cc2N(Cc2cc(Cl)ccc2F)C1=O] | (R)-1-(tert-butyl)-3-(4-(5-chloro-2-fluorobenzyl)-6- cyano-2-methyl-3-oxo-3,4-dihydro-2H- benzo[b][1,4]oxazin-7-yl)urea |
| | C₂₂H₂₂ClFN₄O₃ | |
| 374 | 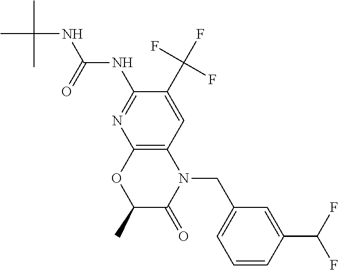 [SMILES: C[C@H]1Oc2nc(NC(=O)NC(C)(C)C)c(C(F)(F)F)cc2N(Cc2cccc(C(F)F)c2)C1=O] | (R)-1-(tert-butyl)-3-(1-(3-(difluoromethyl)benzyl)-3- methyl-2-oxo-7-(trifluoromethyl)-2,3-dihydro-1H- pyrido[2,3-b][1,4]oxazin-6-yl)urea |
| | C₂₂H₂₃F₅N₄O₃ | |
| 375 | 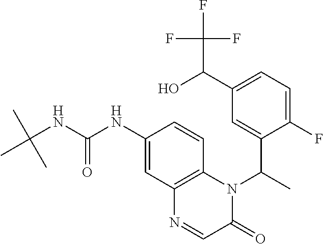 [SMILES: CC(c1cc(C(O)C(F)(F)F)ccc1F)n1c(=O)cnc2cc(NC(=O)NC(C)(C)C)ccc21] | 1-(tert-butyl)-3-(1-(1-(2-fluoro-5-(2,2,2-trifluoro-1- hydroxyethyl)phenyl)ethyl)-2-oxo-1,2-dihydroquinoxalin- 6-yl)urea |
| | C₂₃H₂₄F₄N₄O₃ | |
| 376 | 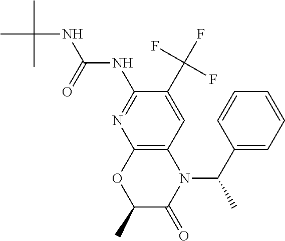 [SMILES: C[C@H]1Oc2nc(NC(=O)NC(C)(C)C)c(C(F)(F)F)cc2N([C@@H](C)c2ccccc2)C1=O] | 1-(tert-butyl)-3-((R)-3-methyl-2-oxo-1-((S)-1- phenylethyl)-7-(trifluoromethyl)-2,3-dihydro-1H- pyrido[2,3-b][1,4]oxazin-6-yl)urea |
| | C₂₂H₂₅F₃N₄O₃ | |
| 377 | 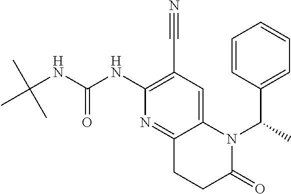 [SMILES: C[C@@H](c1ccccc1)N1C(=O)CCc2nc(NC(=O)NC(C)(C)C)c(C#N)cc21] | (S)-1-(tert-butyl)-3-(6-oxo-5-(1-phenylethyl)-3- (trifluoromethyl)-5,6,7,8-tetrahydro-1,5-naphthyridin-2- yl)urea |
| | C₂₂H₂₅F₃N₄O₂ | |
| 378 | 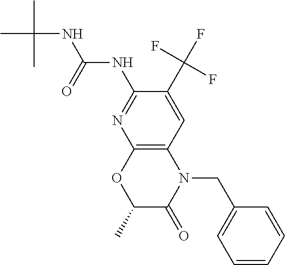 [SMILES: C[C@@H]1Oc2nc(NC(=O)NC(C)(C)C)c(C(F)(F)F)cc2N(Cc2ccccc2)C1=O] | (R)-1-(1-benzyl-3-methyl-2-oxo-7-(trifluoromethyl)-2,3- dihydro-1H-pyrido[2,3-b][1,4]oxazin-6-yl)-3-(tert- butyl)urea |
| | C₂₁H₂₃F₃N₄O₃ | |

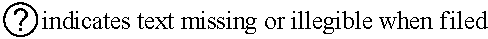

In certain embodiments, the compound is selected from Table 2.

TABLE 2

| | | |
|---|---|---|
| 1 | 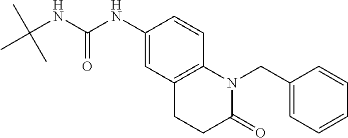 [SMILES: CC(C)(C)NC(=O)Nc1ccc2c(c1)CCC(=O)N2Cc1ccccc1] | 1-(1-benzyl-2-oxo-1,2,3,4- tetrahydroquinolin- 6-yl)-3-(tert-butyl)urea |
| 2 | 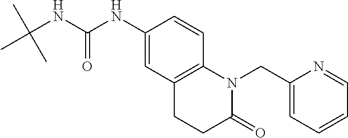 [SMILES: CC(C)(C)NC(=O)Nc1ccc2c(c1)CCC(=O)N2Cc1ccccn1] | 1-(tert-butyl)-3- (2-oxo-1-(pyridin-2-ylmethyl)- 1,2,3,4-tetrahydroquinolin-6-yl)urea |
| 3 | 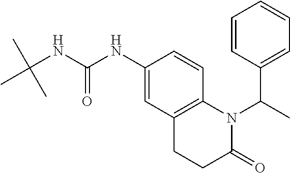 [SMILES: CC(c1ccccc1)N1C(=O)CCc2cc(NC(=O)NC(C)(C)C)ccc21] | 1-(tert-butyl)-3-(2-oxo- 1-(1-phenylethyl)-1,2,3,4- tetrahydroquinolin-6-yl)urea |
| 4 | 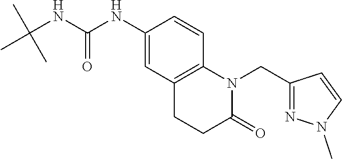 [SMILES: Cn1ccc(CN2C(=O)CCc3cc(NC(=O)NC(C)(C)C)ccc32)n1] | 1-(tert-butyl)-3- (1-((1-methyl-1H-pyrazol-3-yl)methyl)- 2-oxo-1,2,3,4-tetrahydroquinolin-6-yl)urea |
| 5 | 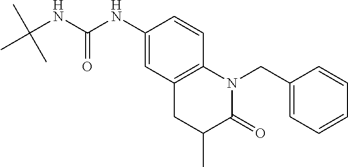 [SMILES: CC1Cc2cc(NC(=O)NC(C)(C)C)ccc2N(Cc2ccccc2)C1=O] | 1-(1-benzyl-3-methyl-2- oxo-1,2,3,4-tetrahydroquinolin-6-yl)-3- (tert-butyl)urea |
| 6 | 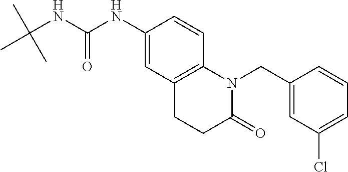 [SMILES: CC(C)(C)NC(=O)Nc1ccc2c(c1)CCC(=O)N2Cc1cccc(Cl)c1] | 1-(tert-butyl)-3-(1-(3-chlorobenzyl)- 2-oxo-1,2,3,4-tetrahydroquinolin-6-yl)urea |
| 7 | 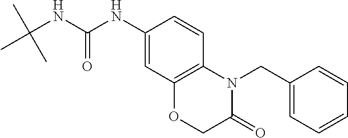 [SMILES: CC(C)(C)NC(=O)Nc1ccc2c(c1)OCC(=O)N2Cc1ccccc1] | 1-(4-benzyl-3-oxo-3,4-dihydro- 2H-benzo[b][1,4]oxazin-7-yl)-3- (tert-butyl)urea |
| 8 | 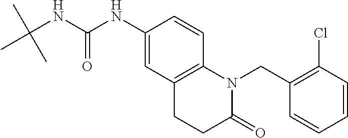 [SMILES: CC(C)(C)NC(=O)Nc1ccc2c(c1)CCC(=O)N2Cc1ccccc1Cl] | 1-(tert-butyl)-3-(1-(2-chlorobenzyl)- 2-oxo-1,2,3,4-tetrahydroquinolin-6-yl)urea |
| 9 | 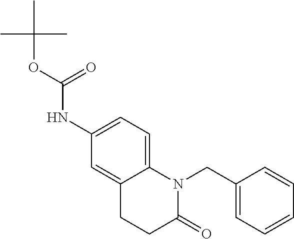 [SMILES: CC(C)(C)OC(=O)Nc1ccc2c(c1)CCC(=O)N2Cc1ccccc1] | tert-butyl (1-benzyl-2-oxo-1,2,3,4- tetrahydroquinolin-6-yl)carbamate |
| 10 | 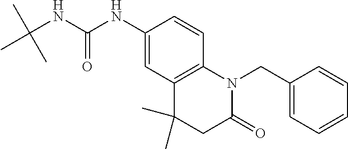 [SMILES: CC(C)(C)NC(=O)Nc1ccc2c(c1)C(C)(C)CC(=O)N2Cc1ccccc1] | 1-(1-benzyl-4,4-dimethyl-2-oxo- 1,2,3,4-tetrahydroquinolin-6-yl)-3- (tert-butyl)urea |
| 11 | 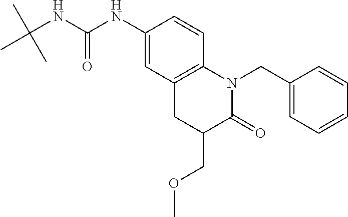 [SMILES: COCC1Cc2cc(NC(=O)NC(C)(C)C)ccc2N(Cc2ccccc2)C1=O] | 1-(1-benzyl-3-(methoxymethyl)- 2-oxo-1,2,3,4-tetrahydroquinolin-6-yl)- 3-(tert-butyl)urea |
| 12 | 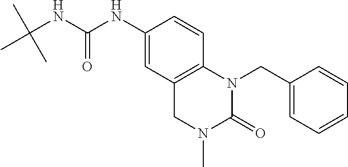 [SMILES: CN1Cc2cc(NC(=O)NC(C)(C)C)ccc2N(Cc2ccccc2)C1=O] | 1-(1-benzyl-3-methyl-2- oxo-1,2,3,4-tetrahydroquinazolin-6-yl)- 3-(tert-butyl)urea |
| 13 | 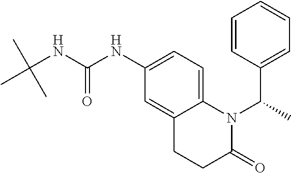 [SMILES: C[C@@H](c1ccccc1)N1C(=O)CCc2cc(NC(=O)NC(C)(C)C)ccc21] | (S)-1-(tert-butyl)-3- (2-oxo-1-(1-phenylethyl)- 1,2,3,4-tetrahydroquinolin-6-yl)urea |
| 14 | 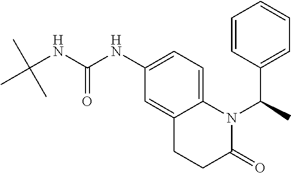 [SMILES: C[C@H](c1ccccc1)N1C(=O)CCc2cc(NC(=O)NC(C)(C)C)ccc21] | (R)-1-(tert-butyl)-3- (2-oxo-1-(1-phenylethyl)-1,2,3,4- tetrahydroquinolin-6-yl)urea |
| 15 | 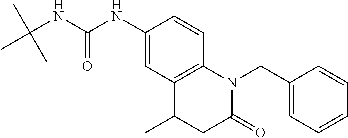 [SMILES: CC1CC(=O)N(Cc2ccccc2)c2ccc(NC(=O)NC(C)(C)C)cc21] | 1-(1-benzyl-4-methyl-2-oxo- 1,2,3,4-tetrahydroquinolin-6-yl)- 3-(tert-butyl)urea |
| 16 | 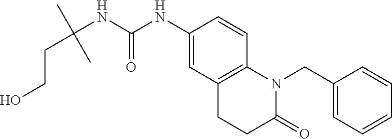 [SMILES: CC(C)(CCO)NC(=O)Nc1ccc2c(c1)CCC(=O)N2Cc1ccccc1] | 1-(1-benzyl-2-oxo-1,2,3,4- tetrahydroquinolin-6-yl)-3- (4-hydroxy-2-methylbutan-2-yl)urea |
| 17 | 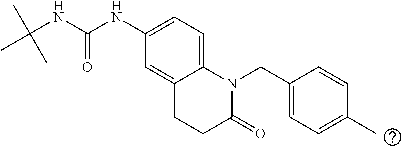 [SMILES: Cc1ccc(CN2C(=O)CCc3cc(NC(=O)NC(C)(C)C)ccc32)cc1] | 1-(tert-butyl)-3- (1-(4-cyanobenzyl)-2-oxo- 1,2,3,4-tetrahydroquinolin-6-yl)urea |
| 18 | 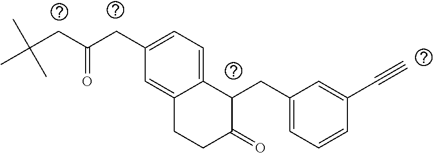 [SMILES: C#Cc1cccc(CC2C(=O)CCc3cc(CC(=O)CC(C)(C)C)ccc32)c1] | 1-(tert-butyl)-3- (1-(3-cyanobenzyl)-2-oxo- 1,2,3,4-tetrahydroquinolin-6-yl)urea |
| 19 | 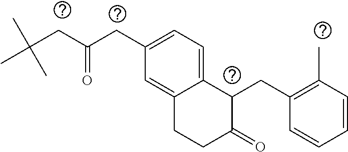 [SMILES: Cc1ccccc1CC1C(=O)CCc2cc(CC(=O)CC(C)(C)C)ccc21] | 1-(tert-butyl)-3- (1-(2-fluorobenzyl)-2-oxo- 1,2,3,4-tetrahydroquinolin-6-yl)urea |
| 20 | 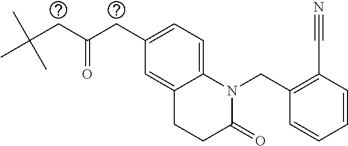 [SMILES: CC(C)(C)CC(=O)Cc1ccc2c(c1)CCC(=O)N2Cc1ccccc1C#N] | 1-(tert-butyl)-3- (1-(2-cyanobenzyl)-2-oxo- 1,2,3,4-tetrahydroquinolin-6-yl)urea |
| 21 | 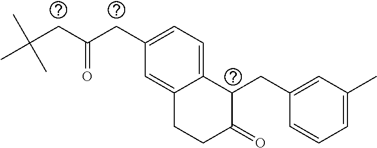 [SMILES: Cc1cccc(CC2C(=O)CCc3cc(CC(=O)CC(C)(C)C)ccc32)c1] | 1-(tert-butyl)-3- (1-(3-methylbenzyl)-2-oxo- 1,2,3,4-tetrahydroquinolin-6-yl)urea |
| 22 | 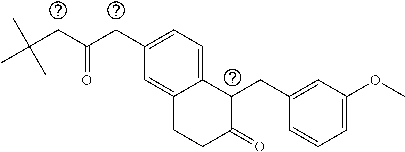 [SMILES: COc1cccc(CC2C(=O)CCc3cc(CC(=O)CC(C)(C)C)ccc32)c1] | 1-(tert-butyl)-3- (1-(3-methoxybenzyl)-2-oxo- 1,2,3,4-tetrahydroquinolin-6-yl)urea |
| 23 | 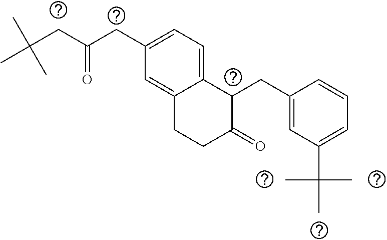 [SMILES: CC(C)(C)CC(=O)Cc1ccc2c(c1)CCC(=O)C2Cc1cccc(C(C)(C)C)c1] | 1-(tert-butyl)-3- (2-oxo-1-(3-(trifluoromethyl)benzyl)- 1,2,3,4-tetrahydroquinolin-6-yl)urea |
| 24 | 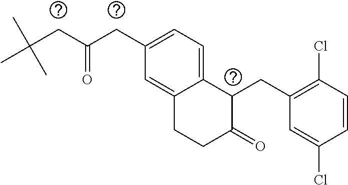 [SMILES: CC(C)(C)CC(=O)Cc1ccc2c(c1)CCC(=O)C2Cc1cc(Cl)ccc1Cl] | 1-(tert-butyl)-3- (1-(2,5-dichlorobenzyl)-2-oxo- 1,2,3,4-tetrahydroquinolin-6-yl)urea |
| 25 | 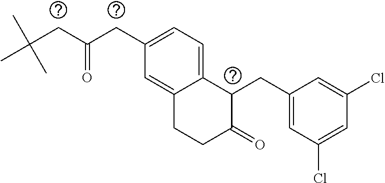 [SMILES: CC(C)(C)CC(=O)Cc1ccc2c(c1)CCC(=O)C2Cc1cc(Cl)cc(Cl)c1] | 1-(tert-butyl)-3- (1-(3,5-dichlorobenzyl)-2-oxo-1,2,3,4- tetrahydroquinolin-6-yl)urea |
| 26 | 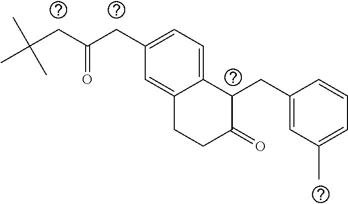 [SMILES: Cc1cccc(CC2C(=O)CCc3cc(CC(=O)CC(C)(C)C)ccc32)c1] | 1-(tert-butyl)-3-(1-(3-fluorobenzyl)-2-oxo- 1,2,3,4-tetrahydroquinolin-6-yl)urea |
| 27 | 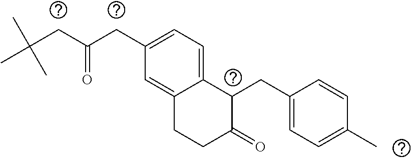 [SMILES: Cc1ccc(CC2C(=O)CCc3cc(CC(=O)CC(C)(C)C)ccc32)cc1] | 1-(tert-butyl)-3-(1-(4-fluorobenzyl)-2-oxo- 1,2,3,4-tetrahydroquinolin-6-yl)urea |
| 28 | 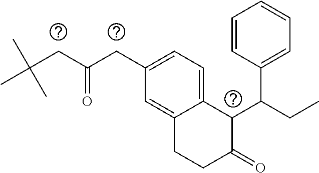 [SMILES: CCC(c1ccccc1)C1C(=O)CCc2cc(CC(=O)CC(C)(C)C)ccc21] | 1-(tert-butyl)-3-(2-oxo-1-(1-phenylpropyl)- 1,2,3,4-tetrahydroquinolin-6-yl)urea |
| 29 | 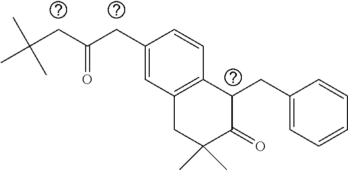 [SMILES: CC(C)(C)CC(=O)Cc1ccc2c(c1)CC(C)(C)C(=O)C2Cc1ccccc1] | 1-(1-benzyl-3,3-dimethyl-2-oxo-1,2,3,4- tetrahydroquinolin-6-yl)-3-(tert-butyl)urea |
| 30 | 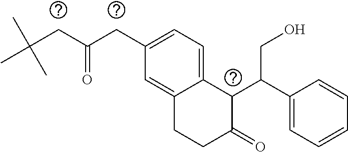 [SMILES: CC(C)(C)CC(=O)Cc1ccc2c(c1)CCC(=O)C2C(CO)c1ccccc1] | 1-(tert-butyl)-3-(1-(2-hydroxy-1-phenylethyl)- 2-oxo-1,2,3,4-tetrahydroquinolin-6-yl)urea |
| 31 | 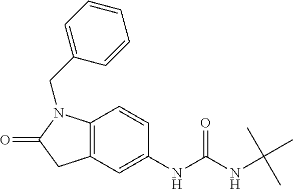 [SMILES: CC(C)(C)NC(=O)Nc1ccc2c(c1)CC(=O)N2Cc1ccccc1] | 1-(1-benzyl-2-oxoindolin-5-yl)-3-(tert-butyl)urea |
| 32 | 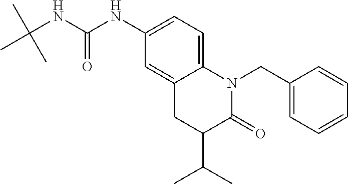 [SMILES: CC(C)C1Cc2cc(NC(=O)NC(C)(C)C)ccc2N(Cc2ccccc2)C1=O] | 1-(1-benzyl-3-isopropyl-2-oxo- 1,2,3,4-tetrahydroquinolin-6-yl)-3- (tert-butyl)urea |
| 33 | 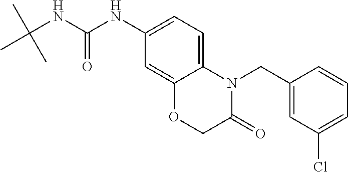 [SMILES: CC(C)(C)NC(=O)Nc1ccc2c(c1)OCC(=O)N2Cc1cccc(Cl)c1] | 1-(tert-butyl)-3-(4-(3-chlorobenzyl)- 3-oxo-3,4-dihydro-2H-benzo[b][1,4] oxazin-7-yl)urea |
| 34 | 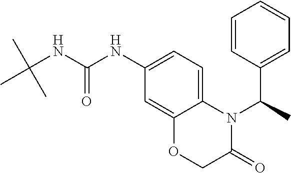 [SMILES: C[C@H](c1ccccc1)N1C(=O)COc2cc(NC(=O)NC(C)(C)C)ccc21] | (R)-1-(tert-butyl)-3-(3-oxo-4- (1-phenylethyl)-3,4-dihydro-2H- benzo[b][1,4]oxazin-7-yl)urea |
| 35 | 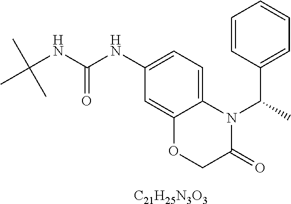 | (S)-1-(tert-butyl)-3- (3-oxo-4-(1-phenylethyl)-3,4- dihydro-2H-benzo[b][1,4] oxazin-7-yl)urea |
| 36 | 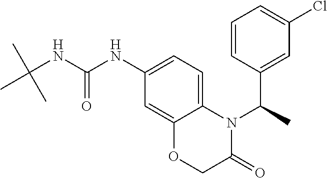 [SMILES: C[C@H](c1cccc(Cl)c1)N1C(=O)COc2cc(NC(=O)NC(C)(C)C)ccc21] | (R)-1-(tert-butyl)-3- (4-(1-(3-chlorophenyl)ethyl)- 3-oxo-3,4-dihydro- 2H-benzo[b][1,4]oxazin-7-yl)urea |
| 37 | 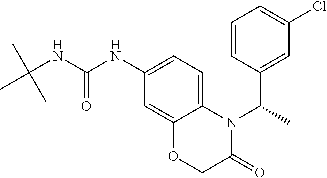 [SMILES: C[C@@H](c1cccc(Cl)c1)N1C(=O)COc2cc(NC(=O)NC(C)(C)C)ccc21] | (S)-1-(tert-butyl)-3- (4-(1-(3-chlorophenyl)ethyl)-3-oxo- 3,4-dihydro-2H-benzo[b][1,4] oxazin-7-yl)urea |
| 38 | 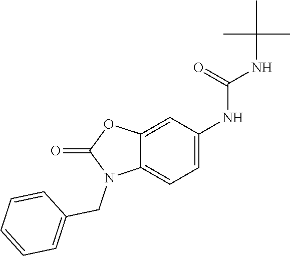 [SMILES: CC(C)(C)NC(=O)Nc1ccc2c(c1)oc(=O)n2Cc1ccccc1] | 1-(3-benzyl-2-oxo-2,3- dihydrobenzo[d]oxazol-6-yl)-3- (tert-butyl)urea |
| 39 | 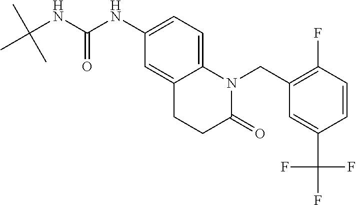 [SMILES: CC(C)(C)NC(=O)Nc1ccc2c(c1)CCC(=O)N2Cc1cc(C(F)(F)F)ccc1F] | 1-(tert-butyl)-3- (1-(2-fluoro-5-(trifluoromethyl)benzyl)- 2-oxo-1,2,3,4-tetrahydroquinolin-6-yl)urea |
| 40 | 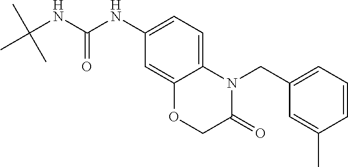 [SMILES: Cc1cccc(CN2C(=O)COc3cc(NC(=O)NC(C)(C)C)ccc32)c1] | 1-(tert-butyl)-3-(4-(3-methylbenzyl)- 3-oxo-3,4-dihydro-2H-benzo[b][1,4] oxazin-7-yl)urea |
| 41 | 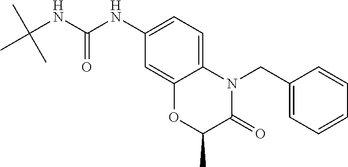 [SMILES: C[C@H]1Oc2cc(NC(=O)NC(C)(C)C)ccc2N(Cc2ccccc2)C1=O] | (R)-1-(4-benzyl-2-methyl-3-oxo-3,4- dihydro-2H-benzo[b][1,4]oxazin-7-yl)-3- (tert-butyl)urea |
| 42 | 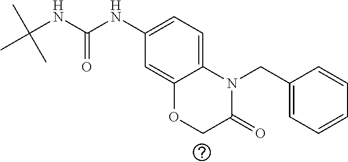 [SMILES: CC(C)(C)NC(=O)Nc1ccc2c(c1)OCC(=O)N2Cc1ccccc1] | (S)-1-(4-benzyl-2-methyl-3-oxo-3,4- dihydro-2H-benzo[b][1,4]oxazin-7-yl)-3- (tert-butyl)urea |
| 43 | 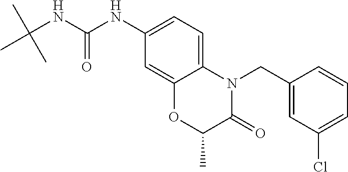 [SMILES: C[C@@H]1Oc2cc(NC(=O)NC(C)(C)C)ccc2N(Cc2cccc(Cl)c2)C1=O] | (S)-1-(tert-butyl)-3-(4-(3-chlorobenzyl)- 2-methyl-3-oxo-3,4-dihydro-2H- benzo[b][1,4]oxazin-7-yl)urea |
| 44 | 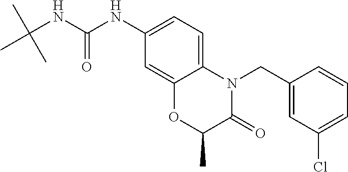 [SMILES: C[C@H]1Oc2cc(NC(=O)NC(C)(C)C)ccc2N(Cc2cccc(Cl)c2)C1=O] | (R)-1-(tert-butyl)-3- (4-(3-chlorobenzyl)-2-methyl-3-oxo- 3,4-dihydro-2H-benzo[b][1,4]oxazin-7-yl)urea |
| 45 | 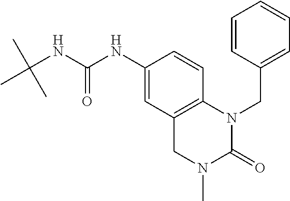 [SMILES: CN1Cc2cc(NC(=O)NC(C)(C)C)ccc2N(Cc2ccccc2)C1=O] | (S)-1-(tert-butyl)-3- (3-methyl-2-oxo-1-(1-phenylethyl)- 1,2,3,4-tetrahydroquinazolin-6-yl)urea |
| 46 | 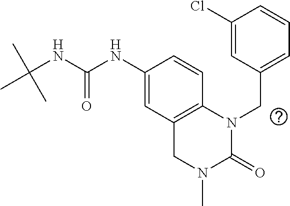 [SMILES: CN1Cc2cc(NC(=O)NC(C)(C)C)ccc2N(Cc2cccc(Cl)c2)C1=O] | (S)-1-(tert-butyl)-3-(1-(1- (3-chlorophenyl)ethyl)-3-methyl-2-oxo- 1,2,3,4-tetrahydroquinazolin-6-yl)urea |
| 47 | 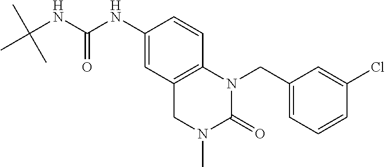 [SMILES: CN1Cc2cc(NC(=O)NC(C)(C)C)ccc2N(Cc2cccc(Cl)c2)C1=O] | 1-(tert-butyl)-3-(1-(3-chlorobenzyl)- 3-methyl-2-oxo- 1,2,3,4-tetrahydroquinazolin-6-yl)urea |
| 48 | 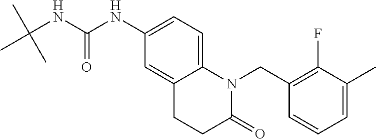 [SMILES: Cc1cccc(CN2C(=O)CCc3cc(NC(=O)NC(C)(C)C)ccc32)c1F] | 1-(tert-butyl)-3-(1-(2-fluoro-3-methylbenzyl)- 2-oxo-1,2,3,4-tetrahydroquinolin-6-yl)urea |
| 49 | 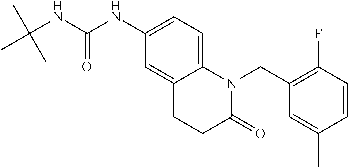 [SMILES: Cc1ccc(F)c(CN2C(=O)CCc3cc(NC(=O)NC(C)(C)C)ccc32)c1] | 1-(tert-butyl)-3-(1-(2-fluoro-5-methylbenzyl)- 2-oxo-1,2,3,4-tetrahydroquinolin-6-yl)urea |
| 50 | 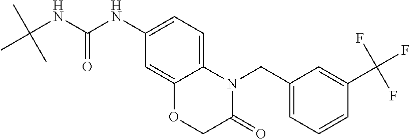 [SMILES: CC(C)(C)NC(=O)Nc1ccc2c(c1)OCC(=O)N2Cc1cccc(C(F)(F)F)c1] | 1-(tert-butyl)-3-(3-oxo-4-(3- (trifluoromethyl)benzyl)- 3,4-dihydro-2H-benzo[b] [1,4]oxazin-7-yl)urea |
| 51 | 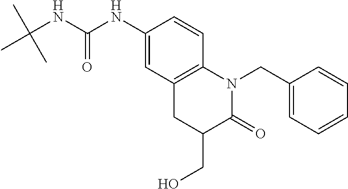 [SMILES: CC(C)(C)NC(=O)Nc1ccc2c(c1)CC(CO)C(=O)N2Cc1ccccc1] | 1-(1-benzyl-3-(hydroxymethyl)-2-oxo-1,2,3,4- tetrahydroquinolin-6-yl)-3-(tert-butyl)urea |
| 52 | 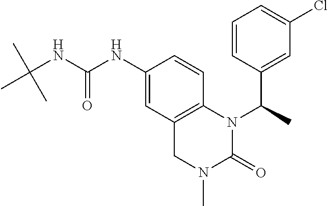 [SMILES: C[C@H](c1cccc(Cl)c1)N1C(=O)N(C)Cc2cc(NC(=O)NC(C)(C)C)ccc21] | (R)-1-(tert-butyl)-3-(1-(1-(3-chlorophenyl)ethyl)- 3-methyl-2-oxo- 1,2,3,4-tetrahydroquinazolin-6-yl)urea |
| 53 | 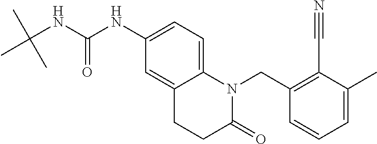 [SMILES: Cc1cccc(CN2C(=O)CCc3cc(NC(=O)NC(C)(C)C)ccc32)c1C#N] | 1-(tert-butyl)-3- (1-(2-cyano-3-methylbenzyl)-2-oxo- 1,2,3,4-tetrahydroquinolin-6-yl)urea |
| 54 | 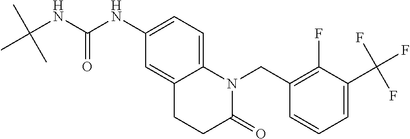 [SMILES: CC(C)(C)NC(=O)Nc1ccc2c(c1)CCC(=O)N2Cc1cccc(C(F)(F)F)c1F] | 1-(tert-butyl)-3-(1-(2-fluoro-3- (trifluoromethyl)benzyl)- 2-oxo-1,2,3,4- tetrahydroquinolin-6-yl)urea |
| 55 | 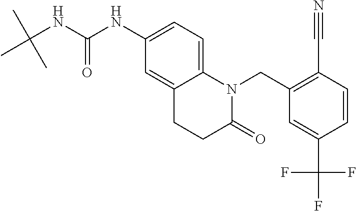 [SMILES: CC(C)(C)NC(=O)Nc1ccc2c(c1)CCC(=O)N2Cc1cc(C(F)(F)F)ccc1C#N] | 1-(tert-butyl)-3-(1-(2-cyano- 5-(trifluoromethyl)benzyl)- 2-oxo-1,2,3,4-tetrahydroquinolin-6-yl)urea |
| 56 | 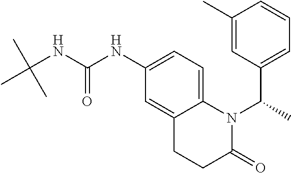 [SMILES: Cc1cccc([C@H](C)N2C(=O)CCc3cc(NC(=O)NC(C)(C)C)ccc32)c1] | (S)-1-(tert-butyl)-3-(2-oxo-1-(1-(m-tolyl)ethyl)- 1,2,3,4-tetrahydroquinolin-6-yl)urea |
| 57 | 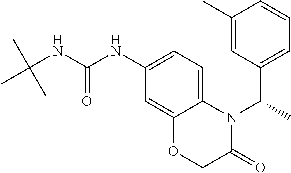 [SMILES: Cc1cccc([C@H](C)N2C(=O)COc3cc(NC(=O)NC(C)(C)C)ccc32)c1] | (S)-1-(tert-butyl)-3-(3-oxo-4-(1-(m-tolyl)ethyl)- 3,4-dihydro-2H-benzo[b][1,4]oxazin-7-yl)urea |
| 58 | 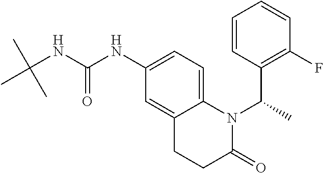 [SMILES: C[C@@H](c1ccccc1F)N1C(=O)CCc2cc(NC(=O)NC(C)(C)C)ccc21] | (S)-1-(tert-butyl)-3-(1-(1- (2-fluorophenyl)ethyl)- 2-oxo-1,2,3,4-tetrahydroquinolin-6-yl)urea |
| 59 | 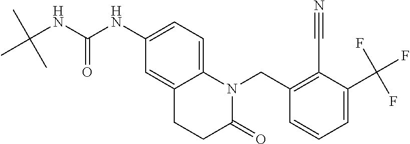 [SMILES: CC(C)(C)NC(=O)Nc1ccc2c(c1)CCC(=O)N2Cc1cccc(C(F)(F)F)c1C#N] | 1-(tert-butyl)-3-(1-(2-cyano-3- (trifluoromethyl)benzyl)- 2-oxo-1,2,3,4- tetrahydroquinolin-6-yl)urea |
| 60 | 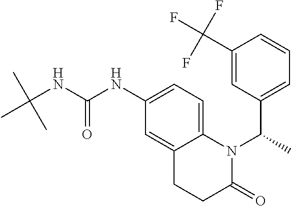 [SMILES: C[C@@H](c1cccc(C(F)(F)F)c1)N1C(=O)CCc2cc(NC(=O)NC(C)(C)C)ccc21] | (S)-1-(tert-butyl)-3-(2-oxo-1-(1-(3- (trifluoromethyl)phenyl)ethyl)- 1,2,3,4-tetrahydroquinolin-6-yl)urea |
| 61 | 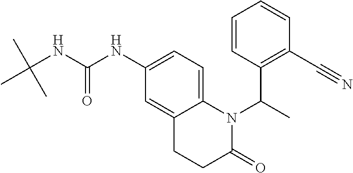 [SMILES: CC(c1ccccc1C#N)N1C(=O)CCc2cc(NC(=O)NC(C)(C)C)ccc21] | (S)-1-(tert-butyl)-3-(1-(1- (2-cyanophenyl)ethyl)-2-oxo- 1,2,3,4-tetrahydroquinolin-6-yl)urea |
| 62 | 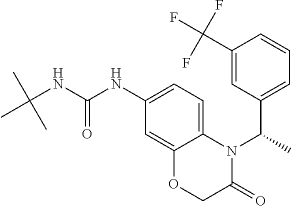 [SMILES: C[C@@H](c1cccc(C(F)(F)F)c1)N1C(=O)COc2cc(NC(=O)NC(C)(C)C)ccc21] | (S)-1-(tert-butyl)-3-(3-oxo-4- (1-(3-(trifluoromethyl)phenyl)ethyl)- 3,4-dihydro-2H-benzo[b][1,4]oxazin-7-yl)urea |
| 63 | 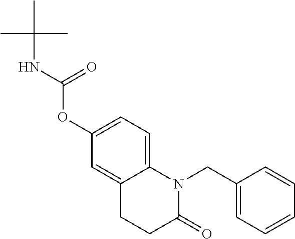 [SMILES: CC(C)(C)NC(=O)Oc1ccc2c(c1)CCC(=O)N2Cc1ccccc1] | 1-benzyl-2-oxo-1,2,3,4- tetrahydroquinolin-6-yl tert-butylcarbamate |
| 64 | 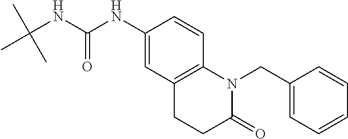 [SMILES: CC(C)(C)NC(=O)Nc1ccc2c(c1)CCC(=O)N2Cc1ccccc1] | 1-(tert-butyl)-3-(1-(cyclohexylmethyl)-2- oxo-1,2,3,4-tetrahydroquinolin-6-yl)urea |
| 65 | 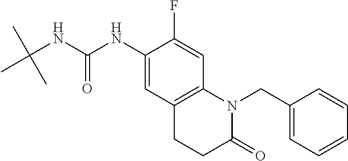 [SMILES: CC(C)(C)NC(=O)Nc1cc2c(cc1F)N(Cc1ccccc1)C(=O)CC2] | 1-(1-benzyl-7-fluoro-2-oxo- 1,2,3,4-tetrahydroquinolin-6-yl)- 3-(tert-butyl)urea |
| 66 | 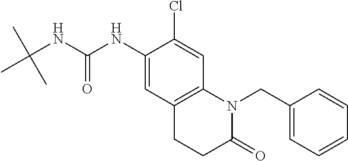 [SMILES: CC(C)(C)NC(=O)Nc1cc2c(cc1Cl)N(Cc1ccccc1)C(=O)CC2] | 1-(1-benzyl-7-chloro-2-oxo- 1,2,3,4-tetrahydroquinolin-6-yl)- 3-(tert-butyl)urea |
| 67 | 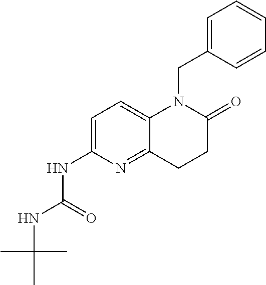 [SMILES: CC(C)(C)NC(=O)Nc1ccc2c(n1)CCC(=O)N2Cc1ccccc1] | 1-(5-benzyl-6-oxo-5,6,7,8-tetrahydro- 1,5-naphthyridin-2-yl)-3-(tert-butyl)urea |
| 68 | 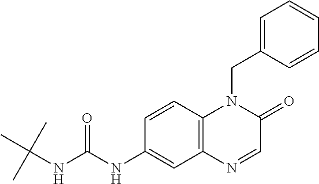 [SMILES: CC(C)(C)NC(=O)Nc1ccc2c(c1)ncc(=O)n2Cc1ccccc1] | 1-(1-benzyl-2-oxo-1,2-dihydroquinoxalin- 6-yl)-3-(tert-butyl)urea |
| 69 | 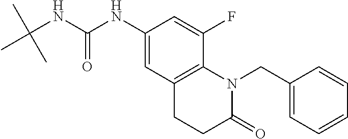 [SMILES: CC(C)(C)NC(=O)Nc1cc(F)c2c(c1)CCC(=O)N2Cc1ccccc1] | 1-(1-benzyl-8-fluoro-2-oxo-1,2,3,4- tetrahydroquinolin-6-yl)-3-(tert-butyl)urea; and |
| 70 | 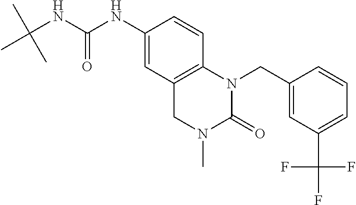 [SMILES: CN1Cc2cc(NC(=O)NC(C)(C)C)ccc2N(Cc2cccc(C(F)(F)F)c2)C1=O] | 1-(tert-butyl)-3-(3-methyl-2-oxo-1-(3- (trifluoromethyl)benzyl)-1,2,3,4- tetrahydroquinazolin-6-yl)urea |

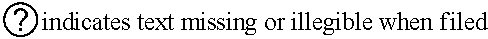

or a pharmaceutically acceptable salt thereof.

In certain embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R¹, the (C₁-C₄)alkyl or the —O—(C₁-C₄)alkyl can be suitably deuterated (e.g., -CD₃, —OCD₃).

Any compound of the invention can also be radiolabed for the preparation of a radiopharmaceutical agent.

### Methods of Treatment

One aspect of the invention provides methods of inhibiting a thyroid stimulating hormone receptor, comprising administering to a subject in need thereof a therapeutically effective amount of a compound described herein.

Another aspect of the invention comprises treating or preventing a thyroid disease, comprising administering to a subject in need thereof a therapeutically effective amount of a compound described herein.

In certain embodiments, the subject has Graves' disease.

In certain embodiments, the subject has Graves' ophthalmopathy.

In certain embodiments, the subject has Graves' dermopathy.

In certain embodiments, the subject has thyroid cancer.

While not being bound by theory, TSHR in thyroid cells, and likely in fibroblasts and adipocytes in the supporting tissue behind the eye (in the retro-orbital space), also are stimulated by TSHR-stimulating antibodies (TSAbs), resulting in Graves' disease. Graves' disease, which is an autoimmune disease that occurs in 1% of the US population, has two important clinical components: 1) hyperthyroidism from stimulation of TSHR on thyroid cells and 2) Graves' orbitopathy (or Graves' ophthalmopathy or thyroid eye disease), which appears to result from stimulation of TSHR on retro-orbital fibroblasts and/or adipocytes.

Hyperthyroidism, in particular Graves' hyperthyroidism, is a hypermetabolic state that affects virtually every tissue/cell in the body and can lead to, in particular, cardiovascular dysfunction and death.

Graves' ophthalmopathy, also known as Graves' orbitopathy, occurs in 80% of Graves' hyperthyroid patients as diagnosed by computerized tomographic scan. Symptoms range from mild to moderate to severe to sight-threatening. Protrusion of the eyeball (proptosis) and varying degrees of extra-ocular muscle weakness or paralysis leading to double vision (diplopia) can be disfiguring and incapacitating.

Graves dermopathy, also known as Pretibial myxedema, thyroid dermopathy, Jadassohn-Dossekker disease or Myxoedema tuberosum, is an infiltrative dermopathy, resulting as a complication of Graves' disease, with an incidence rate of about 1-5% in patients. The disease usually presents itself as a waxy, discolored induration of the skin on the anterior aspect of the lower legs.

In certain embodiments, a disease that can be treated o prevented by TSHR antagonists is thyroid cancer. While not being bound by theory, TSHR is expressed in thyroid cancer cells and regulates the growth, proliferation and metastatic potential of thyroid cancer cells.

The thyroid gland is, as is well known, one site of metabolic control within the body. Cancer of the thyroid gland is not particularly common, but the high rate of disease re-occurrence necessitates long-term surveillance. Usually, during treatment for cancer of the thyroid, the majority of the thyroid tumor is removed, but a small amount often remains that must be treated by radioactive iodide therapy. Indeed, thyroid cancer is characterized by a high likelihood of relapses in up to 30% of patients, even after successful therapy.

In rare cases, the TSHR contains a hereditary mutation that makes it more active than the normal TSHR, resulting in hereditary non-immune hyperthyroidism. TSHR antagonists could be effective treatment for these patients also.

A “TSHR antagonist” as described herein blocks or inhibits the action of the agonists (TSH or thyroid-stimulating antibodies for TSHR. Small-molecule ligands for the TSHR (antagonists) typically bind to an intra-membrane domain of the receptor, and act by inducing a conformational change rather than simply competing for TSH binding to its extracellular site on the receptor.

In certain embodiments, the antagonists may be selective antagonists for TSHR (i.e, the compounds do not activate or modulate other hormone receptors, particularly luteinizing hormone/chorionic gonadotropin receptor (LHCGR) and follicle-stimulating hormone receptor (FSHR)).

In certain embodiments, the antagonists disclosed herein may be used for treating hyperthyroidism in a subject. For example, the antagonists may inhibit mutant TSHRs with higher than normal basal signaling activities (CAMs) that cause an unusual form of hyperthyroidism. In another example, the antagonists may inhibit stimulation by antibodies found in Graves' disease, which is the most common form of hyperthyroidism.

In certain embodiments, the TSHR antagonists are useful for treating TSHR-mediated thyroid cancer or hyperthyroidism by blocking TSHR-stimulating antibodies (TSAbs) in Graves' hyperthyroidism.

In certain embodiments, the compound is administered orally to the subject.

In certain embodiments, the compound is administered parenterally to the subject.

In certain embodiments, the disease is prevented. In other embodiments, the disease is treated.

### Pharmaceutical Compositions, Routes of Administration, and Dosing

In certain embodiments, the invention is directed to a pharmaceutical composition, comprising a compound of the invention, e.g. a compound of Formula (1), and a pharmaceutically acceptable carrier.

In certain embodiments, the invention is directed to a pharmaceutical composition, comprising a compound of any of the disclosed embodiments, and a pharmaceutically acceptable carrier.

In certain embodiments, the invention is directed to a pharmaceutical composition, comprising a compound of Table 1 or 2, and a pharmaceutically acceptable carrier.

In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of ischemia-reperfusion injury.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing a compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

### Example 1: 1-(1-benzyl-2-oxo-1,2,3,4-tetrahydroquinolin-6-yl)-3-(tert-butyl)urea

### Synthetic Scheme

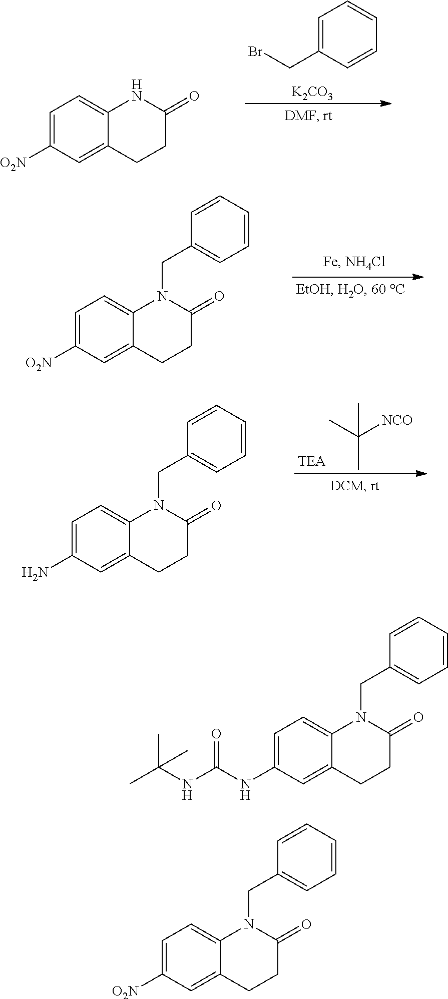

### 1-benzyl-6-nitro-3,4-dihydroquinolin-2(1H)-one

To a solution of 6-nitro-3,4-dihydroquinolin-2(1H)-one (1 g, 5.204 mmol, 1 equiv) in DMF (20 mL) was added K₂CO₃ (2.154 g, 15.611 mmol, 3 equiv) and (bromomethyl)benzene (979 mg, 5.724 mmol, 1.1 equiv). The resulting mixture was stirred for 16 h at rt. The mixture was diluted with EtOAc (100 mL), washed with water (3×50 mL), brine (2×50 mL) and concentrated under vacuum to yield a crude product which was directly purified by silica gel column, eluted with PE/EA (3:1) to afford 1-benzyl-6-nitro-3,4-dihydroquinolin-2(1H)-one (500 mg, 34.04%) as a white solid.

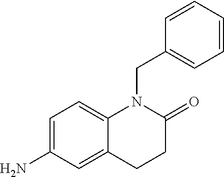 [SMILES: Nc1ccc2c(c1)CCC(=O)N2Cc1ccccc1]

### 6-amino-1-benzyl-3,4-dihydroquinolin-2(1H)-one

To a solution of 1-benzyl-6-nitro-3,4-dihydroquinolin-2(1H)-one (0.45 g, 1.590 mmol, 1 equiv) in EtOH (10 mL) and H₂O (2.5 mL) was added Fe (888.0 mg, 15.90 mmol, 10 equiv) and NH₄Cl (850.5 mg, 15.90 mmol, 10 equiv). The resulting mixture was stirred for 2 h at 60° C. and stirred until the starting material was totally consumed by TLC. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 6-amino-1-benzyl-3,4-dihydroquinolin-2(1H)-one (350 mg, 87.28%) as a yellow solid. MS (ESI): mass calcd. for C₁₆H₁₆N₂O: 252.13 m/z, found 253.10 [M+H]⁺.

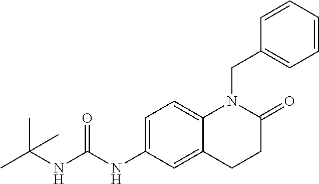 [SMILES: CC(C)(C)NC(=O)Nc1ccc2c(c1)CCC(=O)N2Cc1ccccc1]

### 1-(1-benzyl-2-oxo-1,2,3,4-tetrahydroquinolin-6-yl)-3-(tert-butyl)urea

To a solution of 6-amino-1-benzyl-3,4-dihydroquinolin-2(1H)-one (150 mg, 0.594 mmol, 1 equiv) in DCM (8 mL) was added TEA (179.9 mg, 1.782 mmol, 3 equiv) and 2-isocyanato-2-methylpropane (176.65 mg, 1.782 mmol, 3 equiv). The resulting mixture was stirred for 16 h at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XBridge Prep OBD C18 Column, 30*150 mm, 5 μm; Mobile Phase A: Water(10 mmol/L NH4HCO3), Mobile Phase B: ACN; Flow rate: 60 mL/min; Gradient: 28% B to 53% B in 9 min, 53% B; Wave Length: 254/220 nm; RT1(min): 8.52; Number Of Runs: 0) to afford 1-(1-benzyl-2-oxo-1,2,3,4-tetrahydroquinolin-6-yl)-3-(tert-butyl)urea (60.2 mg, 28.94%) as an off-white solid. MS (ESI): mass calcd. For C₂₁H₂₅N₃O₂, 351.19 m/z, found 352.20 [M+H]⁺. ¹H NMR (400 MHz, DMSO-d₆) δ 8.08 (s, 1H), 7.26-7.34 (m, 3H), 7.16-7.25 (m, 3H), 6.95 (dd, J=8.8, 2.5 Hz, 1H), 6.76 (d, J=8.8 Hz, 1H), 5.90 (s, 1H), 5.09 (s, 2H), 2.87 (dd, J=8.7, 5.9 Hz, 2H), 2.64 (dd, J=8.7, 5.9 Hz, 2H), 1.26 (s, 9H).

### Example 2: 1-(tert-butyl)-3-(2-oxo-1-(pyridin-2-ylmethyl)-1,2,3,4-tetrahydroquinolin-6-yl)urea

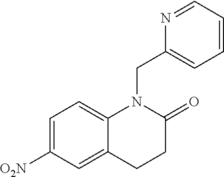 [SMILES: O=C1CCc2cc([N+](=O)[O-])ccc2N1Cc1ccccn1]

### 6-nitro-1-(pyridin-2-ylmethyl)-3,4-dihydroquinolin-2(1H)-one

To a solution of 6-nitro-3,4-dihydro-1H-quinolin-2-one (500 mg, 2.602 mmol, 1 equiv) in DMF (10 mL) was added K₂CO₃ (1078.7 mg, 7.805 mmol, 3 equiv) and 2-(bromomethyl)pyridine (492.3 mg, 2.862 mmol, 1 equiv). The resulting mixture was stirred for 16 h at rt. The mixture was diluted with EtOAc (100 mL), washed with water (3×30 mL), brine (2×30 mL) and concentrated under vacuum to yield a crude product which was directly purified by silica gel column, eluted with PE/EA (3:1) to afford 6-nitro-1-(pyridin-2-ylmethyl)-3,4-dihydroquinolin-2(1H)-one (500 mg, 63.20%) as a white solid. MS (ESI): mass calcd. for C₁₅H₁₃N₃O₃ 283.10 m/z, found 284.05 [M+H]⁺.

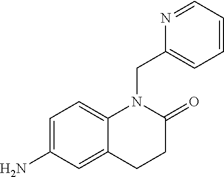 [SMILES: Nc1ccc2c(c1)CCC(=O)N2Cc1ccccn1]

### 6-amino-1-(pyridin-2-ylmethyl)-3,4-dihydroquinolin-2-one

To a solution of 6-nitro-1-(pyridin-2-ylmethyl)-3,4-dihydroquinolin-2-one (440 mg, 1.553 mmol, 1 equiv) in EtOH (10 mL) and H₂O (2.5 mL) was added Fe (867.3 mg, 15.532 mmol, 10 equiv) and NH₄Cl (830.8 mg, 15.530 mmol, 10 equiv). The resulting mixture was stirred for 2 h at 60° C. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 6-amino-1-(pyridin-2-ylmethyl)-3,4-dihydroquinolin-2-one (200 mg, 46.52%) as a yellow solid. MS (ESI): mass calcd. for C₁₅H₁₅N₃O, 253.12 m/z, found 254.15 [M+H]⁺.

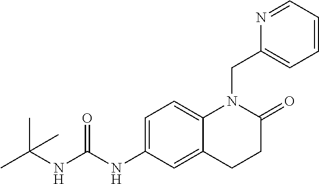 [SMILES: CC(C)(C)NC(=O)Nc1ccc2c(c1)CCC(=O)N2Cc1ccccn1]

### 1-(tert-butyl)-3-(2-oxo-1-(pyridin-2-ylmethyl)-1,2,3,4-tetrahydroquinolin-6-yl)urea

To a solution of 6-amino-1-(pyridin-2-ylmethyl)-3,4-dihydroquinolin-2(1H)-one (200 mg, 0.793 mmol, 1 equiv) in DCM (5 mL) was added 2-isocyanato-2-methylpropane (234.8 mg, 2.369 mmol, 3 equiv) and TEA (79.9 mg, 0.790 mmol, 1 equiv). The resulting mixture was stirred for overnight at rt. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford & Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 22% B to 34% B in 10.5 min; Wave Length: 254/220 nm; RT1(min): 11.3; Number Of Runs: 4) to afford 1-(tert-butyl)-3-(2-oxo-1-(pyridin-2-ylmethyl)-1,2,3,4-tetrahydroquinolin-6-yl)urea (83.1 mg, 29.82%) as a white solid. MS (ESI): mass calcd. for C₂₀H₂₄N₄O₂, 352.19 m/z, found 353.10 [M+H]⁺. ¹H NMR (300 MHz, DMSO-d₆) δ 8.52 (d, J=4.8 Hz, 1H), 8.11 (d, J=3.5 Hz, 1H), 7.71 (d, J=8.4 Hz, 1H), 7.34 (s, 1H), 7.25 (q, J=5.1 Hz, 1H), 7.21-7.21 (m, 1H), 6.97 (d, J=8.8 Hz, 1H), 6.69-6.78 (m, 1H), 5.92 (s, 1H), 5.13 (s, 2H), 2.89 (d, J=8.0 Hz, 2H), 2.64 (d, J=8.4 Hz, 2H), 1.26 (s, 9H).

### Example 3: 1-(tert-butyl)-3-(2-oxo-1-(1-phenylethyl)-1,2,3,4-tetrahydroquinolin-6-yl)urea

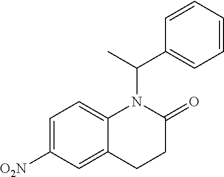 [SMILES: CC(c1ccccc1)N1C(=O)CCc2cc([N+](=O)[O-])ccc21]

### 6-nitro-1-(1-phenylethyl)-3,4-dihydroquinolin-2-one

To a solution of 6-nitro-3,4-dihydro-1H-quinolin-2-one (500 mg, 2.602 mmol, 1 equiv) in DMF (10 mL) was added K₂CO₃ (1078.7 mg, 7.805 mmol, 3 equiv) and (1-bromoethyl)benzene (529.6 mg, 2.862 mmol, 1 equiv). The resulting mixture was stirred for 16 h at rt. The mixture was diluted with EtOAc (100 mL), washed with water (3×50 mL), brine (2×50 mL) and concentrated under vacuum to yield a crude product which was directly purified by silica gel column, eluted with PE/EA (3:1) to afford 6-nitro-1-(1-phenylethyl)-3,4-dihydroquinolin-2-one (400 mg, 45.17%) as a white solid. MS (ESI): mass calcd. for C₁₇H₁₆N₂O₃ 296.12 m/z, found 297.05 [M+H]⁺.

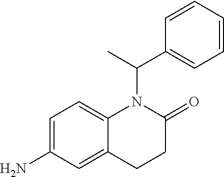 [SMILES: CC(c1ccccc1)N1C(=O)CCc2cc(N)ccc21]

### 6-amino-1-(1-phenylethyl)-3,4-dihydroquinolin-2-one

To a solution of 6-nitro-1-(1-phenylethyl)-3,4-dihydroquinolin-2-one (400 mg, 1.350 mmol, 1 equiv) in EtOH (10 mL) and H₂O (2.5 mL) was added Fe (753.8 mg, 13.499 mmol, 10 equiv) and NH₄Cl (722.0 mg, 13.499 mmol, 10 equiv). The resulting mixture was stirred for 2 h at 60° C. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (3:1) to afford 6-amino-1-(1-phenylethyl)-3,4-dihydroquinolin-2-one (200 mg, 55.63%) as a white solid.

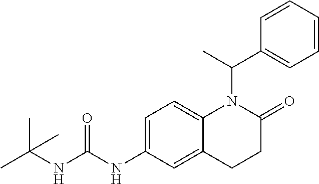 [SMILES: CC(c1ccccc1)N1C(=O)CCc2cc(NC(=O)NC(C)(C)C)ccc21]

### 1-(tert-butyl)-3-(2-oxo-1-(1-phenylethyl)-1,2,3,4-tetrahydroquinolin-6-yl)urea

To a solution of 6-amino-1-(1-phenylethyl)-3,4-dihydroquinolin-2-one (200 mg, 0.751 mmol, 1 equiv) in DCM (5 mL) was added 2-isocyanato-2-methylpropane (223.3 mg, 2.253 mmol, 3 equiv) and TEA (75.9 mg, 0.751 mmol, 1 equiv). The resulting mixture was stirred for overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column, eluted with PE/EA (2:1) & Prep-HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 38% B to 50% B in 10.5 min; Wave Length: 254/220 nm; RT1(min): 10.7; Number Of Runs: 3) to afford 1-(tert-butyl)-3-(2-oxo-1-(1-phenylethyl)-1,2,3,4-tetrahydroquinolin-6-yl)urea (57.7 mg, 21.02%) as a white solid. MS (ESI): mass calcd. for C₂₂H₂₇N₃O₂, 365.21 m/z, found 366.15 [M+H]⁺. ¹H NMR (300 MHz, DMSO-d₆) δ 8.07 (s, 1H), 7.18-7.39 (m, 6H), 6.81 (d, J=8.8 Hz, 1H), 6.42-6.52 (m, 1H), 6.15 (d, J=8.0 Hz, 1H), 5.91 (s, 1H), 2.80 (d, J=5.6 Hz, 2H), 2.54-2.70 (m, 2H), 1.67 (t, J=5.7 Hz, 3H), 1.24 (dd, J=4.1, 2.2 Hz, 9H).

### Example 4: 1-(tert-butyl)-3-(1-((1-methyl-1H-pyrazol-3-yl)methyl)-2-oxo-1,2,3,4-tetrahydroquinolin-6-yl)urea

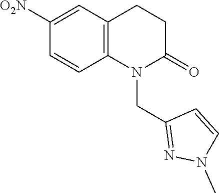 [SMILES: Cn1ccc(CN2C(=O)CCc3cc([N+](=O)[O-])ccc32)n1]

### 1-[(1-methylpyrazol-3-yl)methyl]-6-nitro-3,4-dihydroquinolin-2-one

To a solution of 6-nitro-3,4-dihydro-1H-quinolin-2-one (500 mg, 2.602 mmol, 1 equiv) in DMF (10 mL) was added K₂CO₃ (1086.6 mg, 7.805 mmol, 3 equiv) and 3-(bromomethyl)-1-methylpyrazole (500.9 mg, 2.862 mmol, 1.1 equiv). The resulting mixture was stirred for 16 h at rt. The mixture was diluted with EtOAc (100 mL), washed with water (3×30 mL), brine (2×30 mL) and concentrated under vacuum to yield a crude product which was directly purified by silica gel column, eluted with PE/EA (3:1) to afford 1-[(1-methylpyrazol-3-yl)methyl]-6-nitro-3,4-dihydroquinolin-2-one (500 mg, 46.59%) as a white solid. MS (ESI): mass calcd. for C₁₄H₁₄N₄O₃: 286.11 m/z, found 287.00 [M+H]⁺.

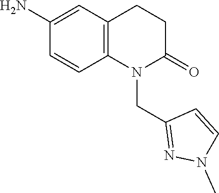 [SMILES: Cn1ccc(CN2C(=O)CCc3cc(N)ccc32)n1]

### 6-amino-1-[(1-methylpyrazol-3-yl)methyl]-3,4-dihydroquinolin-2-one

To a solution of 6-nitro-1-(1H-pyrazol-3-ylmethyl)-3,4-dihydroquinolin-2-one (300 mg, 1.102 mmol, 1 equiv) in EtOH (10 mL) and H₂O (2.5 mL) was added Fe (615.3 mg, 11.019 mmol, 10 equiv) and NH₄Cl (589.3 mg, 11.019 mmol, 10 equiv). The resulting mixture was stirred for 2 h at 60° C. and stirred until the starting material was totally consumed by TLC. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 6-amino-1-[(1-methylpyrazol-3-yl)methyl]-3,4-dihydroquinolin-2-one (101 mg, 36.03%) as a yellow solid.

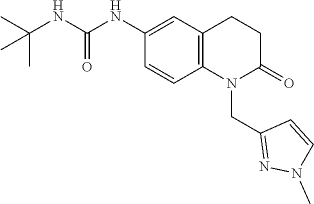 [SMILES: Cn1ccc(CN2C(=O)CCc3cc(NC(=O)NC(C)(C)C)ccc32)n1]

### 1-(tert-butyl)-3-(1-((1-methyl-1H-pyrazol-3-yl)methyl)-2-oxo-1,2,3,4-tetrahydroquinolin-6-yl)urea

To a solution of 6-amino-1-[(1-methylpyrazol-3-yl)methyl]-3,4-dihydroquinolin-2-one (50 mg, 0.195 mmol, 1 equiv) in DCM (5 mL) was added 2-isocyanato-2-methylpropane (58.0 mg, 0.585 mmol, 3 equiv) and TEA (19.7 mg, 0.195 mmol, 1 equiv). The resulting mixture was stirred for overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column, eluted with PE/EA (2:1) & Prep-HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 1-(tert-butyl)-3-(1-((1-methyl-1H-pyrazol-3-yl)methyl)-2-oxo-1,2,3,4-tetrahydroquinolin-6-yl)urea (9.0 mg, 12.23%) as a white solid. MS (ESI): mass calcd. for C₁₉H₂₅N₅O₂, 355.20 m/z, found 356.10 [M+H]⁺. ¹H NMR (300 MHz, DMSO-d₆) δ 8.10 (d, J=5.2 Hz, 1H), 7.48-7.62 (m, 1H), 7.29 (s, 1H), 7.01 (s, 2H), 5.85-6.04 (m, 2H), 4.95 (s, 2H), 3.78-3.82 (m, 3H), 2.81 (s, 2H), 2.51 (s, 2H), 1.17-1.37 (m, 9H).

### Example 5: 1-(1-benzyl-3-methyl-2-oxo-1,2,3,4-tetrahydroquinolin-6-yl)-3-(tert-butyl)urea

### Synthetic Scheme

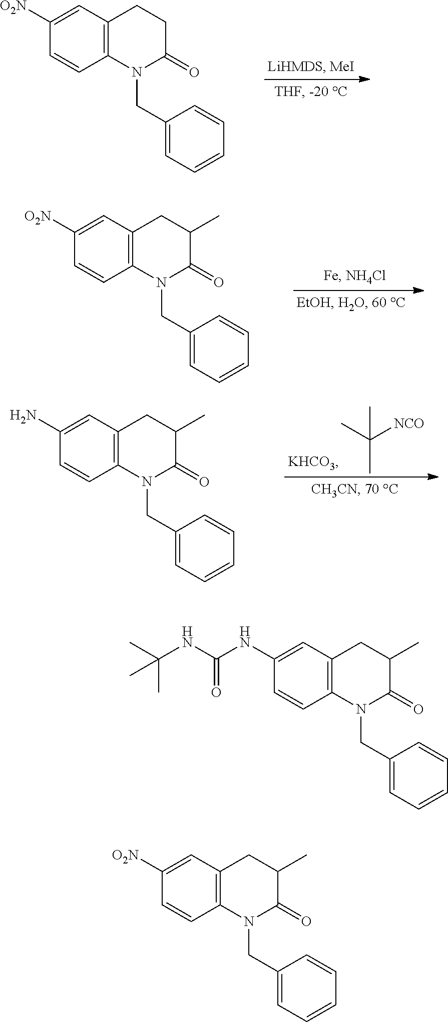

### 1-benzyl-3-methyl-6-nitro-3,4-dihydroquinolin-2(1H)-one

To a solution of 1-benzyl-6-nitro-3,4-dihydroquinolin-2(1H)-one (1 g, 3.54 mmol, 1 equiv) in THF (70 mL) was added 2M LiHMDS (in THF) (2.3 mL, 4.60 mmol, 1.3 equiv) at −20° C. under N₂. The resulting mixture was stirred 30 min at −20° C. The reaction mixture was added MeI (653.6 mg, 4.60 mmol, 1.3 equiv) at −20° C. The resulting mixture was stirred 30 min at −20° C. and then stirred 16 h at rt. The reaction mixture was quenched by water (50 mL) and extracted with EA (3×70 mL). The combined organic extracts were washed with brine (50 mL), dried over anhydrous Na₂SO₄, and concentrated under vacuum to yield a crude product which was directly purified by silica gel column, eluted with PE/EA (3:1) to afford 1-benzyl-3-methyl-6-nitro-3,4-dihydroquinolin-2(1H)-one (0.3 g, 28.59%) as a white solid.

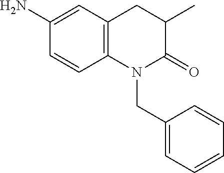 [SMILES: CC1Cc2cc(N)ccc2N(Cc2ccccc2)C1=O]

### 6-amino-1-benzyl-3-methyl-3,4-dihydroquinolin-2(1H)-one

To a solution of 1-benzyl-3-methyl-6-nitro-3,4-dihydroquinolin-2(1H)-one (0.28 g, 0.944 mmol, 1 equiv) in EtOH (10 mL) and H₂O (2.5 mL) was added Fe (527.7 mg, 9.44 mmol, 10 equiv) and NH₄Cl (504.9 mg, 9.76 mmol, 10 equiv). The resulting mixture was stirred for 2 h at 60° C. and stirred until the starting material was totally consumed by TLC. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 3-((6-amino-2-oxo-3,4-dihydroquinolin-1(2H)-yl)methyl)benzonitrile (160 mg, 46.37%) as a yellow solid. MS (ESI): mass calcd. for C₁₇H₁₈N₂O: 266.14 m/z, found 267.20 [M+H]⁺.

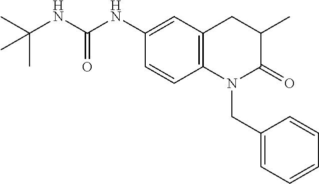 [SMILES: CC1Cc2cc(NC(=O)NC(C)(C)C)ccc2N(Cc2ccccc2)C1=O]

### 1-(tert-butyl)-3-(1-(3-cyanobenzyl)-2-oxo-1,2,3,4-tetrahydroquinolin-6-yl)urea

To a solution of 6-amino-1-benzyl-3-methyl-3,4-dihydroquinolin-2(1H)-one (150 mg, 0.563 mmol, 1 equiv) in CH₃CN (5 mL) was added 2-isocyanato-2-methylpropane (167.48 mg, 1.689 mmol, 3 equiv) and KHCO₃ (112.7 mg, 1.126 mmol, 2 equiv). The resulting mixture was stirred for overnight at 70° C. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column, eluted with PE/EA (2:1) & Prep-HPLC (Column: XBridge Prep OBD C18 Column, 30*150 mm, 5 μm; Mobile Phase A: Water(10 mmol/L NH4HCO3), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 1-(tert-butyl)-3-(1-(3-cyanobenzyl)-2-oxo-1,2,3,4-tetrahydroquinolin-6-yl)urea (18.1 mg, 8.78%) as a white solid. MS (ESI): mass calcd. for C₂₂H₂₇N₃O₂: 365.21 m/z, found 366.10 [M+H]⁺. ¹H NMR (400 MHz, Chloroform-d) δ 7.26-7.31 (m, 2H), 7.12-7.23 (m, 3H), 6.82 (d, J=8.7 Hz, 1H), 6.71 (d, J=8.6 Hz, 1H), 6.25-6.54 (m, 1H), 5.19 (d, J=16.3 Hz, 1H), 5.05 (d, J=16.0 Hz, 1H), 2.94 (d, J=11.6 Hz, 1H), 2.58-2.81 (m, 2H), 1.33 (s, 9H), 1.30 (d, J=6.2 Hz, 3H).

### Example 6: 1-(tert-butyl)-3-(1-(3-chlorobenzyl)-2-oxo-1,2,3,4-tetrahydroquinolin-6-yl)urea

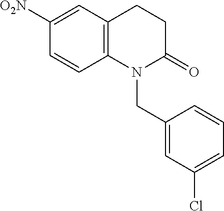 [SMILES: O=C1CCc2cc([N+](=O)[O-])ccc2N1Cc1cccc(Cl)c1]

### 1-(3-chlorobenzyl)-6-nitro-3,4-dihydroquinolin-2(1H)-one

To a solution of 6-nitro-3,4-dihydro-1H-quinolin-2-one (500 mg, 2.602 mmol, 1 equiv) in dimethylformamide (10 mL) was added 1-(bromomethyl)-3-chlorobenzene (801.93 mg, 3.903 mmol, 1.5 equiv) and potassium methaneperoxoate potassium (724.41 mg, 5.204 mmol, 2.0 equiv) at rt. The mixture was stirred for overnight at rt. The reaction mixture was quenched by water (30 mL) and extracted with EA (3×30 mL). The combined organic extracts were washed with brine, dried over anhydrous Na₂SO₄, and concentrated under vacuum. The residue was purified by silica gel column chromatography, eluted with PE/EA (3:1) to afford 1-(3-chlorobenzyl)-6-nitro-3,4-dihydroquinolin-2(1H)-one (700 mg, 88.29%) as a white solid. MS (ESI): mass calcd. C₁₆H₁₃ClN₂O₃: for 316.06 m/z, found 317.10[M+H]⁺.

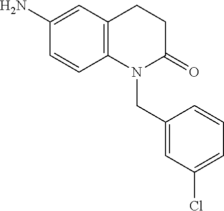 [SMILES: Nc1ccc2c(c1)CCC(=O)N2Cc1cccc(Cl)c1]

### 6-amino-1-(3-chlorobenzyl)-3,4-dihydroquinolin-2(1H)-one

To a solution of 1-(3-chlorobenzyl)-6-nitro-3,4-dihydroquinolin-2(1H)-one (700 mg, 2.210 mmol, 1 equiv) in EtOH (5 mL)/H₂O (0.5 mL) was added Fe (1.2 g, 22.1 mmol, 10 equiv) and NH₄Cl (1.2 g, 22.100 mmol, 10 equiv) at rt. The mixture was stirred for 2 h at 60° C. The reaction mixture was quenched by water (30 mL) and extracted with EA (3×30 mL). The combined organic extracts were washed with brine, dried over anhydrous Na₂SO₄, and concentrated under vacuum. The residue was purified by silica gel column chromatography, eluted with PE/EA (2:1) to afford 6-amino-1-(3-chlorobenzyl)-3,4-dihydroquinolin-2(1H)-one (600 mg, 94.68%) as a brown solid. MS (ESI): mass calcd. C₁₆H₁₅ClN₂O: for 286.09 m/z, found 287.10[M+H]⁺.

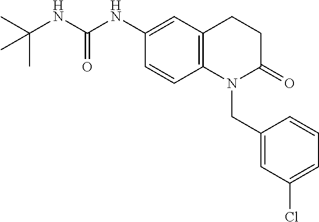 [SMILES: CC(C)(C)NC(=O)Nc1ccc2c(c1)CCC(=O)N2Cc1cccc(Cl)c1]

### 1-(tert-butyl)-3-(1-(3-chlorobenzyl)-2-oxo-1,2,3,4-tetrahydroquinolin-6-yl)urea

A solution of 6-amino-1-(3-chlorobenzyl)-3,4-dihydroquinolin-2(1H)-one (50 mg, 0.174 mmol, 1 equiv) in DCM (3 mL) was added TEA (35.29 mg, 0.348 mmol, 2.0 equiv) and 2-isocyanato-2-methylpropane (20.74 mg, 0.209 mmol, 1.2 equiv) at 0° C. The resulting mixture was stirred for 16 h at rt. The reaction mixture was quenched by water (20 mL). The resulting mixture was extracted with DCM (3×30 mL). The combined organic layers were washed with brine, dried over anhydrous Na₂SO₄. After filtration, the filtrate was concentrated under reduced pressure. The residue was purified by silica gel column chromatography, eluted with PE/EA (2:1) to afford 1-(tert-butyl)-3-(1-(3-chlorobenzyl)-2-oxo-1,2,3,4-tetrahydroquinolin-6-yl)urea (30.4 mg, 45.11%) as white solid. MS (ESI): mass calcd. C₂₁H₂₄ClN₃O₂ for: 385.16 m/z, found 386.1 [M+H]⁺. ¹H NMR (300 MHz, DMSO-d₆) δ 8.12 (s, 1H), 7.24-7.40 (m, 4H), 7.15-7.18 (m, 1H), 6.96-7.01 (m, 1H), 6.75-6.80 (m, 1H), 5.92 (s, 1H), 5.10 (s, 2H), 2.85-2.92 (m, 2H), 2.61-2.70 (m, 2H), 1.27 (s, 9H).

### Example 7: 1-(4-benzyl-3-oxo-3,4-dihydro-2H-benzo[b][1,4]oxazin-7-yl)-3-(tert-butyl)urea

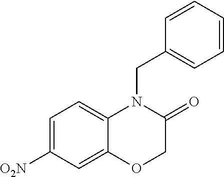 [SMILES: O=C1COc2cc([N+](=O)[O-])ccc2N1Cc1ccccc1]

### 4-benzyl-7-nitro-2H-1,4-benzoxazin-3-one

To a solution of 7-nitro-2,4-dihydro-1,4-benzoxazin-3-one (400 mg, 2.060 mmol, 1 equiv) in DMF (10 mL) was added K₂CO₃ (860.4 mg, 6.181 mmol, 3 equiv) and benzyl bromide (387.6 mg, 2.266 mmol, 1.1 equiv). The resulting mixture was stirred for 16 h at rt. The mixture was diluted with EtOAc (100 mL), washed with water (3×30 mL), brine (2×30 mL) and concentrated under vacuum to yield a crude product which was directly purified by silica gel column, eluted with PE/EA (3:1) to afford 4-benzyl-7-nitro-2H-1,4-benzoxazin-3-one (500 mg, 72.97%) as a yellow solid. MS (ESI): mass calcd. for C₁₅H₁₂N₂O₄ 284.08 m/z, found 285.05 [M+H]⁺.

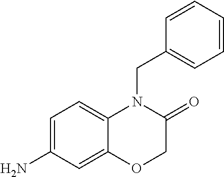 [SMILES: Nc1ccc2c(c1)OCC(=O)N2Cc1ccccc1]

### 7-amino-4-benzyl-2H-1,4-benzoxazin-3-one

To a solution of 4-benzyl-7-nitro-2H-1,4-benzoxazin-3-one (500 mg, 1.759 mmol, 1 equiv) in EtOH (10 mL) and H₂O (2.5 mL) was added Fe (982.2 mg, 17.589 mmol, 10 equiv) and NH₄Cl (940.8 mg, 17.589 mmol, 10 equiv). The resulting mixture was stirred for 2 h at 60° C. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 7-amino-4-benzyl-2H-1,4-benzoxazin-3-one (200 mg, 44.72%) as a yellow solid. MS (ESI): mass calcd. for C₁₅H₁₄N₂O₂, 254.11 m/z, found 255.05 [M+H]⁺.

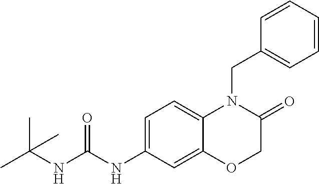 [SMILES: CC(C)(C)NC(=O)Nc1ccc2c(c1)OCC(=O)N2Cc1ccccc1]

### 1-(4-benzyl-3-oxo-3,4-dihydro-2H-benzo[b][1,4]oxazin-7-yl)-3-(tert-butyl)urea

To a solution of 7-amino-4-benzyl-2H-1,4-benzoxazin-3-one (150 mg, 0.590 mmol, 1 equiv) in DCM (5 mL) was added 2-isocyanato-2-methylpropane (175.4 mg, 1.770 mmol, 3 equiv) and TEA (59.6 mg, 0.590 mmol, 1 equiv). The resulting mixture was stirred for overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column, eluted with PE/EA (2:1) & Prep-HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 1-(4-benzyl-3-oxo-3,4-dihydro-2H-benzo[b][1,4]oxazin-7-yl)-3-(tert-butyl)urea (56.8 mg, 27.14%) as a white solid. MS (ESI): mass calcd. for C₂₀H₂₃N₃O₃, 353.17 m/z, found 354.15 [M+H]⁺. ¹H NMR (300 MHz, DMSO-d6) δ 8.21 (s, 1H), 7.17-7.41 (m, 7H), 6.86 (dd, J=8.6, 2.1 Hz, 1H), 6.71-6.82 (m, 1H), 5.93 (s, 1H), 5.11 (s, 2H), 4.74 (d, J=2.1 Hz, 2H), 1.26 (d, J=2.1 Hz, 9H).

### Example 8: 1-(tert-butyl)-3-(1-(2-chlorobenzyl)-2-oxo-1,2,3,4-tetrahydroquinolin-6-yl)urea

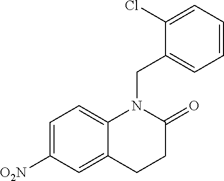 [SMILES: O=C1CCc2cc([N+](=O)[O-])ccc2N1Cc1ccccc1Cl]

### 1-(2-chlorobenzyl)-6-nitro-3,4-dihydroquinolin-2(1H)-one

To a solution of 6-nitro-3,4-dihydroquinolin-2(1H)-one (500 mg, 2.602 mmol, 1 equiv) in DMF (10 mL) was added K₂CO₃ (1086.6 mg, 7.805 mmol, 3 equiv) and 1-(bromomethyl)-2-chlorobenzene (583.64 mg, 2.862 mmol, 1.1 equiv). The resulting mixture was stirred for 16 h at rt. The mixture was diluted with EtOAc (50 mL), washed with water (3×30 mL), brine (2×30 mL) and concentrated under vacuum to yield a crude product which was directly purified by silica gel column, eluted with PE/EA (3:1) to afford 1-(2-chlorobenzyl)-6-nitro-3,4-dihydroquinolin-2(1H)-one (400 mg, 48.54%) as a white solid. MS (ESI): mass calcd. for C₁₆H₁₃ClN₂O₃: 316.06 m/z, found 317.00 [M+H]⁺.

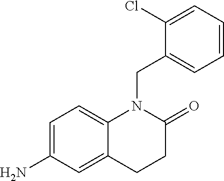 [SMILES: Nc1ccc2c(c1)CCC(=O)N2Cc1ccccc1Cl]

### 6-amino-1-(2-chlorobenzyl)-3,4-dihydroquinolin-2(1H)-one

To a solution of 1-(2-chlorobenzyl)-6-nitro-3,4-dihydroquinolin-2(1H)-one (400 mg, 1.263 mmol, 1 equiv) in EtOH (10 mL) and H₂O (2.5 mL) was added Fe (705.3 mg, 12.63 mmol, 10 equiv) and NH₄Cl (675.6 mg, 12.63 mmol, 10 equiv). The resulting mixture was stirred for 2 h at 60° C. and stirred until the starting material was totally consumed by TLC. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 6-amino-1-(2-chlorobenzyl)-3,4-dihydroquinolin-2(1H)-one (0.3 g, 82.87%) as a yellow solid. MS (ESI): mass calcd. for C₁₆H₁₅ClN₂O: 286.09 m/z, found 287.00 [M+H]⁺.

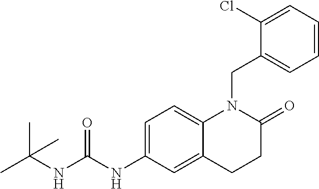 [SMILES: CC(C)(C)NC(=O)Nc1ccc2c(c1)CCC(=O)N2Cc1ccccc1Cl]

### 1-(tert-butyl)-3-(1-(2-chlorobenzyl)-2-oxo-1,2,3,4-tetrahydroquinolin-6-yl)urea

To a solution of 6-amino-1-(2-chlorobenzyl)-3,4-dihydroquinolin-2(1H)-one (100 mg, 0.349 mmol, 1 equiv) in DCM (5 mL) was added 2-isocyanato-2-methylpropane (103.9 mg, 1.048 mmol, 3 equiv) and TEA (105.8 mg, 1.048 mmol, 3 equiv). The resulting mixture was stirred for overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column, eluted with PE/EA (2:1) & Prep-HPLC (Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 1-(tert-butyl)-3-(1-(2-chlorobenzyl)-2-oxo-1,2,3,4-tetrahydroquinolin-6-yl)urea (39.7 mg, 29.40%) as a white solid. MS (ESI): mass calcd. for C₂₁H₂₄ClN₃O₂: 385.16 m/z, found 386.05 [M+H]⁺. ¹H NMR (300 MHz, DMSO-d6) δ 8.12 (s, 1H), 7.47-7.56 (m, 1H), 7.35-7.42 (m, 1H), 7.26 (d, J=8.7 Hz, 2H), 6.92-7.01 (m, 2H), 6.47-6.62 (m, 1H), 5.91 (s, 1H), 5.07 (s, 2H), 2.88-2.96 (m, 2H), 2.63-2.72 (m, 2H), 1.25 (s, 9H).

### Example 9: tert-butyl (1-benzyl-2-oxo-1,2,3,4-tetrahydroquinolin-6-yl)carbamate

### Synthetic Scheme

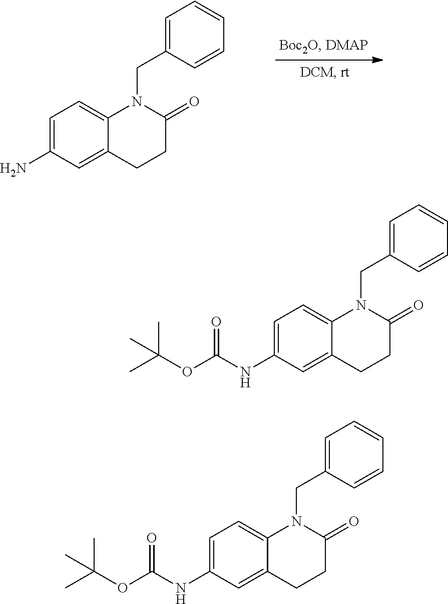 [SMILES: Nc1ccc2c(c1)CCC(=O)N2Cc1ccccc1 ; CC(C)(C)OC(=O)Nc1ccc2c(c1)CCC(=O)N2Cc1ccccc1 ; CC(C)(C)OC(=O)Nc1ccc2c(c1)CCC(=O)N2Cc1ccccc1]

### tert-butyl (1-benzyl-2-oxo-1,2,3,4-tetrahydroquinolin-6-yl)carbamate

To a solution of 6-amino-1-benzyl-3,4-dihydroquinolin-2(1H)-one (100 mg, 0.396 mmol, 1 equiv) in DCM (6 mL) was added Boc2O (129.7 mg, 0.594 mmol, 1.5 equiv) and DMAP (72.6 mg, 0.594 mmol, 1.5 equiv). The resulting mixture was stirred for 5 h at rt. The mixture was diluted with DCM (30 mL), washed with water (3×30 mL), brine (2×30 mL) and concentrated under vacuum to yield a crude product which was directly purified by Prep-HPLC (Column: XBridge Prep OBD C18 Column, 30*150 mm, 5 μm; Mobile Phase A: Water(10 mmol/L NH4HCO3), Mobile Phase B: ACN; Flow rate: 60 mL/min; Gradient: 17% B to 45% B in 9 min, 45% B; Wave Length: 254/220 nm; RT1(min): 8.6; Number Of Runs: 0) to afford tert-butyl (1-benzyl-2-oxo-1,2,3,4-tetrahydroquinolin-6-yl)carbamate (19.4 mg, 10.21%) as a yellow solid. MS (ESI): mass calcd. for C₂₁H₂₄N₂O₃: 352.18 m/z, found 353.10 [M+H]⁺. ¹H NMR (300 MHz, DMSO-d₆) δ 9.21 (s, 1H), 7.39-7.16 (m, 6H), 7.10 (dd, J=9.0, 2.4 Hz, 1H), 6.82 (d, J=8.8 Hz, 1H), 5.11 (s, 2H), 2.88 (t, J=7.2 Hz, 2H), 2.66 (dd, J=8.7, 6.0 Hz, 2H), 1.45 (s, 9H).

### Example 10: 1-(1-benzyl-4,4-dimethyl-2-oxo-1,2,3,4-tetrahydroquinolin-6-yl)-3-(tert-butyl)urea

### Synthetic Scheme

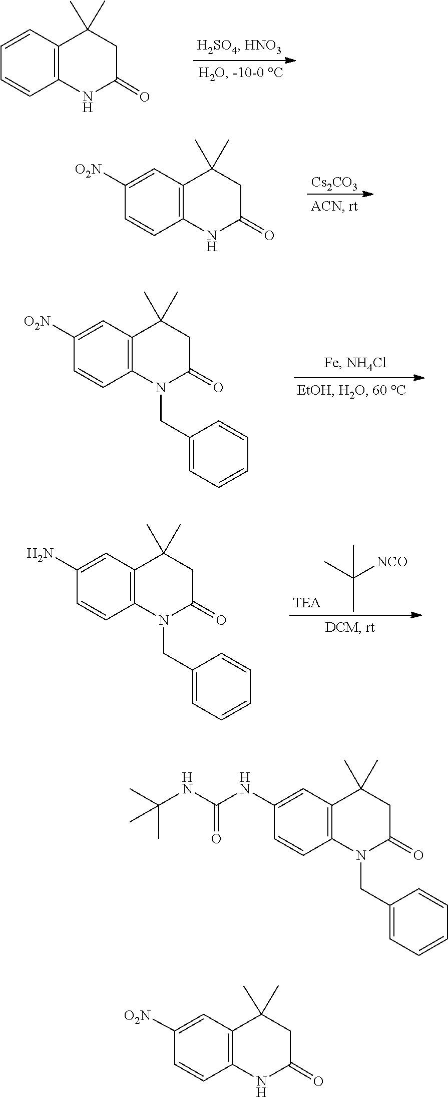

### 4,4-dimethyl-6-nitro-3,4-dihydroquinolin-2(1H)-one

To a solution of 4,4-dimethyl-3,4-dihydroquinolin-2(1H)-one (0.4 g, 2.28 mmol, 1 equiv) in H₂O (2 mL) and H₂SO₄ (9 mL) was added HNO₃ (0.3 mL) at −10° C. The reaction was stirred for 2 h at −10-0° C. The reaction mixture was quenched by water. The mixture was acidified pH=7 with NaHCO₃ (aq.) and then extracted with EA (3×50 mL). The combined organic extracts were washed with brine (50 mL), dried over anhydrous Na₂SO₄, and concentrated under vacuum to yield a crude product which was directly purified by silica gel column, eluted with PE/EA (3:1) to afford 4,4-dimethyl-6-nitro-3,4-dihydroquinolin-2(1H)-one (0.3 g, 59.76%) as a white solid. MS (ESI): mass calcd. for C₁₁H₁₂N₂O₃, 220.08 m/z, found 221.10 [M+H]+.

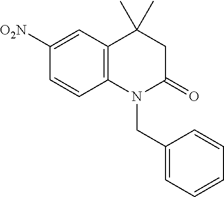 [SMILES: CC1(C)CC(=O)N(Cc2ccccc2)c2ccc([N+](=O)[O-])cc21]

### 1-benzyl-4,4-dimethyl-6-nitro-3,4-dihydroquinolin-2(1H)-one

To a solution of 4,4-dimethyl-6-nitro-3,4-dihydroquinolin-2(1H)-one (280 mg, 1.270 mmol, 1 equiv) in CH₃CN (10 mL) was added Cs₂CO₃ (828.3 mg, 2.542 mmol, 2.0 equiv) and (bromomethyl)benzene (238.9 mg, 1.397 mmol, 1.1 equiv). The resulting mixture was stirred for 16 h at rt. The mixture was diluted with EtOAc (50 mL), washed with water (3×30 mL), brine (2×30 mL) and concentrated under vacuum to yield a crude product which was directly purified by silica gel column, eluted with PE/EA (3:1) to afford 1-benzyl-4,4-dimethyl-6-nitro-3,4-dihydroquinolin-2(1H)-one (300 mg, 76.14%) as a white solid. MS (ESI): mass calcd. for C₁₈H₁₈N₂O₃: 310.13 m/z, found 311.25 [M+H]+.

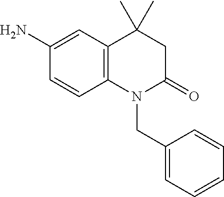 [SMILES: CC1(C)CC(=O)N(Cc2ccccc2)c2ccc(N)cc21]

### 6-amino-1-benzyl-4,4-dimethyl-3,4-dihydroquinolin-2(1H)-one

To a solution of 1-benzyl-4,4-dimethyl-6-nitro-3,4-dihydroquinolin-2(1H)-one (280 mg, 0.902 mmol, 1 equiv) in EtOH (10 mL) and H₂O (2.5 mL) was added Fe (503.9 mg, 9.02 mmol, 10 equiv) and NH₄Cl (482.5 mg, 9.02 mmol, 10 equiv). The resulting mixture was stirred for 2 h at 60° C. and stirred until the starting material was totally consumed by TLC. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 6-amino-1-benzyl-4,4-dimethyl-3,4-dihydroquinolin-2(1H)-one (100 mg, 39.68%) as a yellow solid. MS (ESI): mass calcd. for C₁₈H₂₀N₂O: 280.16 m/z, found 281.20 [M+H]⁺.

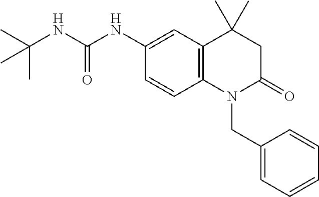 [SMILES: CC(C)(C)NC(=O)Nc1ccc2c(c1)C(C)(C)CC(=O)N2Cc1ccccc1]

### 1-(1-benzyl-4,4-dimethyl-2-oxo-1,2,3,4-tetrahydroquinolin-6-yl)-3-(tert-butyl)urea

To a solution of 6-amino-1-benzyl-4,4-dimethyl-3,4-dihydroquinolin-2(1H)-one (100 mg, 0.357 mmol, 1 equiv) in DCM (5 mL) was added 2-isocyanato-2-methylpropane (106.1 mg, 1.07 mmol, 3 equiv) and TEA (108.1 mg, 1.07 mmol, 3 equiv). The resulting mixture was stirred for overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column, eluted with PE/EA (2:1) & Prep-HPLC (Column: XBridge Prep C18 OBD Column, 30*50 mm, 5 μm 13 nm; Mobile Phase A: Water(10 mmol/L NH₄HCO₃), Mobile Phase B: ACN; Flow rate: 60 mL/min; Gradient: 26% B to 45% B in 9 min, 45% B; Wave Length: 254/220 nm; RT1(min): 8.9; Number Of Runs: 0) to afford 1-(1-benzyl-4,4-dimethyl-2-oxo-1,2,3,4-tetrahydroquinolin-6-yl)-3-(tert-butyl)urea (51.0 mg, 37.50%) as a white solid. MS (ESI): mass calcd. for C₂₃H₂₉N₃O₂: 379.23 m/z, found 380.15 [M+H]⁺. ¹H NMR (300 MHz, DMSO-d₆) δ 8.16 (s, 1H), 7.38 (s, 1H), 7.20-7.31 (m, 5H), 7.02 (s, 1H), 6.86 (s, 1H), 5.88 (s, 1H), 5.13 (s, 2H), 2.50-2.55 (s, 2H), 1.26 (s, 9H), 1.22 (s, 6H).

### Example 11: 1-(1-benzyl-3-(methoxymethyl)-2-oxo-1,2,3,4-tetrahydroquinolin-6-yl)-3-(tert-butyl)urea

### Synthetic Scheme

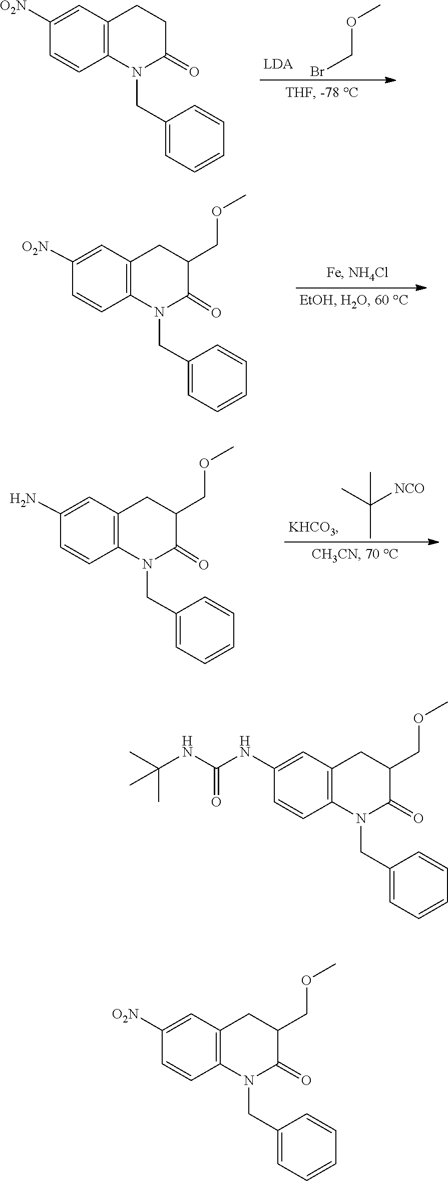

### 1-benzyl-3-(methoxymethyl)-6-nitro-3,4-dihydroquinolin-2-one

To a solution of 1-benzyl-6-nitro-3,4-dihydroquinolin-2-one (1.3 g, 4.605 mmol, 1 equiv) in THF (40 mL) was added 2M LDA (in THF) (3.5 mL, 0.74 g, 6.907 mmol, 1.5 equiv) at −78° C. under N₂. The resulting mixture was stirred 30 min at −78° C. The reaction mixture was added bromo(methoxy)methane (1.15 g, 9.203 mmol, 2.00 equiv) at −78° C. The resulting mixture was stirred for 1 h at −78° C. and 16 h at rt. The reaction mixture was quenched by NH₄Cl (aq.) and extracted with EA (3×50 mL). The combined organic extracts were washed with brine (30 mL), dried over anhydrous Na₂SO₄, and concentrated under vacuum to yield a crude product which was directly purified by silica gel column, eluted with PE/EA (3:1) to afford 1-benzyl-3-(methoxymethyl)-6-nitro-3,4-dihydroquinolin-2-one (400 mg, 12.30%) as a yellow solid. MS (ESI): mass calcd. for C₁₈H₁₈N₂O₄, 326.13 m/z, found 327.05 [M+H]⁺.

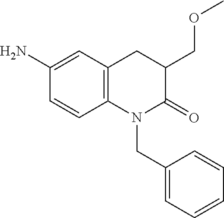 [SMILES: COCC1Cc2cc(N)ccc2N(Cc2ccccc2)C1=O]

### 6-amino-1-benzyl-3-(methoxymethyl)-3,4-dihydroquinolin-2-one

To a solution of 1-benzyl-3-(methoxymethyl)-6-nitro-3,4-dihydroquinolin-2-one (300 mg, 0.919 mmol, 1 equiv) in EtOH (10 mL) and H₂O (2.5 mL) was added Fe (513.3 mg, 9.193 mmol, 10 equiv) and NH₄Cl (491.7 mg, 9.193 mmol, 10 equiv). The resulting mixture was stirred for 2 h at 60° C. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column, eluted with PE/EA (2:1) to afford 6-amino-1-benzyl-3-(methoxymethyl)-3,4-dihydroquinolin-2-one (200 mg, 26.38%) as a yellow solid. MS (ESI): mass calcd. for C₁₈H₂₀N₂O₂, 296.15 m/z, found 297.10 [M+H]⁺.

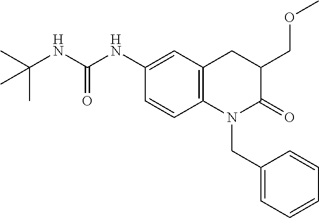 [SMILES: COCC1Cc2cc(NC(=O)NC(C)(C)C)ccc2N(Cc2ccccc2)C1=O]

### 1-(1-benzyl-3-(methoxymethyl)-2-oxo-1,2,3,4-tetrahydroquinolin-6-yl)-3-(tert-butyl)urea

To a solution of 6-amino-1-benzyl-3-(methoxymethyl)-3,4-dihydroquinolin-2-one (100 mg, 0.337 mmol, 1 equiv) in DCM (5 mL) was added 2-isocyanato-2-methylpropane (100.3 mg, 1.012 mmol, 3 equiv) and TEA (102.4 mg, 1.012 mmol, 3 equiv). The resulting mixture was stirred for overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column, eluted with PE/EA (2:1) & Prep-HPLC (Column: XBridge Prep OBD C18 Column, 30*150 mm, 5 m; Mobile Phase A: Water(10 mmol/L NH4HCO3), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 1-(1-benzyl-3-(methoxymethyl)-2-oxo-1,2,3,4-tetrahydroquinolin-6-yl)-3-(tert-butyl)urea (12.1 mg, 8.95%) as a white solid. MS (ESI): mass calcd. for C₂₃H₂₉N₃O₃, 395.22 m/z, found 396.20 [M+H]⁺. ¹H NMR (300 MHz, DMSO-d6) δ 8.01 (s, 1H), 7.19 (d, J=5.5 Hz, 3H), 7.04-7.17 (m, 3H), 6.90 (d, J=8.7 Hz, 1H), 6.66 (d, J=9.1 Hz, 1H), 5.83 (s, 1H), 4.99 (s, 2H), 3.53-3.64 (m, 1H), 3.46 (t, J=8.2 Hz, 1H), 3.20 (d, J=1.7 Hz, 3H), 2.60-2.95 (m, 3H), 1.16 (d, J=1.7 Hz, 9H).

### Example 12: 1-(1-benzyl-3-methyl-2-oxo-1,2,3,4-tetrahydroquinazolin-6-yl)-3-(tert-butyl)urea

### Synthetic Scheme

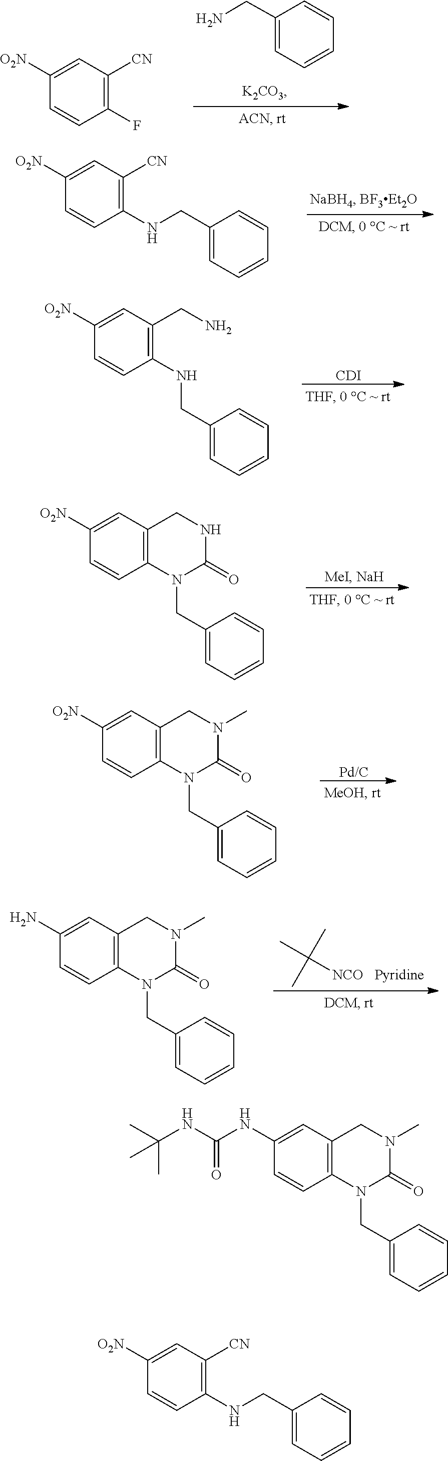

### 2-(benzylamino)-5-nitrobenzonitrile

To a solution of 2-fluoro-5-nitrobenzonitrile (5 g, 30.100 mmol, 1 equiv) in ACN (100 mL) was added benzylamine (4.84 g, 45.150 mmol, 1.5 equiv) and K₂CO₃ (8.32 g, 60.200 mmol, 2 equiv). The reaction was stirred at rt for 1 hour. Quenched with water (20 mL) and extracted with EA (3×20 mL). The combined extracts were washed with water, brine, dried over Na₂SO₄, filtered, and concentrated. The residue was purified by column chromatography on silica gel with EA/PE (0-100%) to give 2-(benzylamino)-5-nitrobenzonitrile (6.875 g, 90.18%) as a yellow solid. MS(ESI): mass calcd. for C₁₄H₁₁N₃O₂: 253.26 m/z, found 254.00[M+H]⁺.

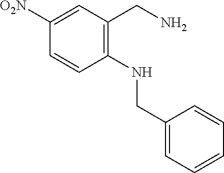 [SMILES: NCc1cc([N+](=O)[O-])ccc1NCc1ccccc1]

### 2-(aminomethyl)-N-benzyl-4-nitroaniline

To a solution of 2-(benzylamino)-5-nitrobenzonitrile (400 mg, 1.579 mmol, 1 equiv) in THF (20 mL) was added NaBH₄ (238.99 mg, 6.316 mmol, 4 equiv) and Boron trifluoride diethyl etherate (896.66 mg, 6.316 mmol, 4 equiv). The reaction was stirred at 0° C. for 0.5 h. The reaction was quenched with water (10 mL) and extracted with EA (3×10 mL). The combined extracts were washed with water, brine, dried over Na₂SO₄, filtered, and concentrated. The residue was purified by column chromatography on silica gel with DCM/MeOH (0-30%) to give 2-(aminomethyl)-N-benzyl-4-nitroaniline (41 mg, 10.09%) as a yellow solid. MS (ESI): mass calcd for C₁₄H₁₅N₃O₂: 257.29 m/z, found 258.10 [M+H]⁺.

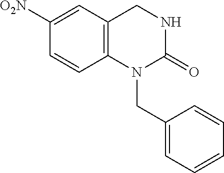 [SMILES: O=C1NCc2cc([N+](=O)[O-])ccc2N1Cc1ccccc1]

### 1-benzyl-6-nitro-3,4-dihydroquinazolin-2(1H)-one

To a solution of 2-(aminomethyl)-N-benzyl-4-nitroaniline (260 mg, 1.011 mmol, 1 equiv) in THF (20 mL) was added CDI (163.86 mg, 1.011 mmol, 1 equiv) at 0° C. The reaction was stirred at rt for 15 min. To the above mixture was added CDI (327.71 mg, 2.022 mmol, 2 equiv) dropwise at 0° C. The resulting mixture was stirred for additional 15 min at rt. After the final reaction mixture was stirred for 18 h at 70° C. Quenched with water (10 mL) and extracted with EA (3×10 mL). The combined extracts were washed with water, brine, dried over Na₂SO₄, filtered, and concentrated. The residue was purified by column chromatography on silica gel with EA/PE (0-100%) to give 1-benzyl-6-nitro-3,4-dihydroquinazolin-2-one (198 mg, 69.17%) as a white solid. MS (ESI): mass calcd. for C₁₅H₁₃N₃O₃: 283.29 m/z, found 284.05 [M+H]⁺.

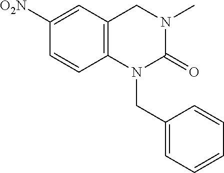 [SMILES: CN1Cc2cc([N+](=O)[O-])ccc2N(Cc2ccccc2)C1=O]

### 1-benzyl-3-methyl-6-nitro-3,4-dihydroquinazolin-2(1H)-one

To a solution of 1-benzyl-6-nitro-3,4-dihydroquinazolin-2-one (184 mg, 0.650 mmol, 1 equiv) in THF (20 mL) was added NaH (23.38 mg, 0.975 mmol, 1.5 equiv) at 0° C. The reaction was stirred at 0° C. for 30 min. To the above mixture was added MeI (184.38 mg, 1.300 mmol, 2 equiv) dropwise at 0° C. The final reaction mixture was stirred for 1 h at rt. Quenched with water (50 mL) and extracted with EA (3×50 mL). The combined extracts were washed with water, brine, dried over Na₂SO₄, filtered, and concentrated. The residue was purified by column chromatography on silica gel with EA/PE (0-100%) to give 1-benzyl-3-methyl-6-nitro-4H-quinazolin-2-one (122 mg, 63.18%) as a white solid. MS (ESI): mass calcd. for C₁₆H₁₅N₃O₃: 297.31 m/z, found 298.00 [M+H]⁺.

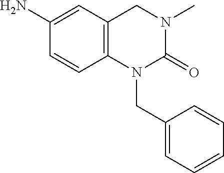 [SMILES: CN1Cc2cc(N)ccc2N(Cc2ccccc2)C1=O]

### 6-amino-1-benzyl-3-methyl-3,4-dihydroquinazolin-2(1H)-one

To a solution of 1-benzyl-3-methyl-6-nitro-4H-quinazolin-2-one (122 mg, 0.410 mmol, 1 equiv) in MeOH (10 mL) was added Pd/C (10%, 122 mg) at rt. The mixture was hydrogenated at room temperature under 30 psi of hydrogen pressure for 1 h. The resulting mixture was filtered, the filter cake was washed with MeOH (3×10 mL). The filtrate was concentrated under reduced pressure. This resulted in 6-amino-1-benzyl-3-methyl-4H-quinazolin-2-one (110 mg, 100.28%) as a white solid. MS (ESI): mass calcd. for C₁₆H₁₇N₃O: 267.33 m/z, found 268.20 [M+H]⁺.

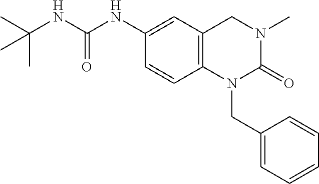 [SMILES: CN1Cc2cc(NC(=O)NC(C)(C)C)ccc2N(Cc2ccccc2)C1=O]

### 1-(1-benzyl-3-methyl-2-oxo-1,2,3,4-tetrahydroquinazolin-6-yl)-3-(tert-butyl)urea

To a solution of 6-amino-1-benzyl-3-methyl-4H-quinazolin-2-one (100 mg, 0.374 mmol, 1 equiv) in DCM (10 mL, 157.306 mmol, 420.53 equiv) was added 2-isocyanato-2-methylpropane (370.82 mg, 3.740 mmol, 10 equiv) and Pyridine (295.89 mg, 3.740 mmol, 10 equiv). The reaction was stirred at rt for 3 days. Quenched with water (10 mL) and extracted with EA (3×10 mL). The combined extracts were washed with water, brine, dried over Na₂S04, filtered, and concentrated. The residue was purified by preparative HPLC using a YMC-Actus Triart C18, 30*150 mm, 5 μm column (eluent: 36% to 56% (v/v) CH₃CN and H₂O with 10 mmol/L NH₄HCO₃) to afford the title compound 1-(1-benzyl-3-methyl-2-oxo-4H-quinazolin-6-yl)-3-tert-butylurea (23.2 mg, 16.92%) as a white solid. MS (ESI): mass calcd. for C₂₁H₂₆N₄O₂:366.47 m/z, found:367.20 [M+H]⁺. ¹H NMR (400 MHz, DMSO-d6) δ 7.90-8.21 (m, 1H), 7.18-7.39 (m, 6H), 6.80-7.00 (m, 1H), 6.44-6.68 (m, 1H), 5.72-5.99 (m, 1H), 4.90-5.20 (m, 2H), 4.28-4.51 (m, 2H), 2.80-3.01 (m, 3H), 1.20-1.31 (m, 9H).

### Example 13: (S)-1-(tert-butyl)-3-(2-oxo-1-(1-phenylethyl)-1,2,3,4-tetrahydroquinolin-6-yl)urea

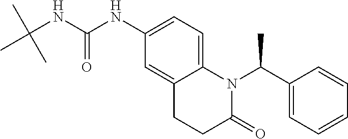 [SMILES: C[C@@H](c1ccccc1)N1C(=O)CCc2cc(NC(=O)NC(C)(C)C)ccc21]

### Synthetic Scheme

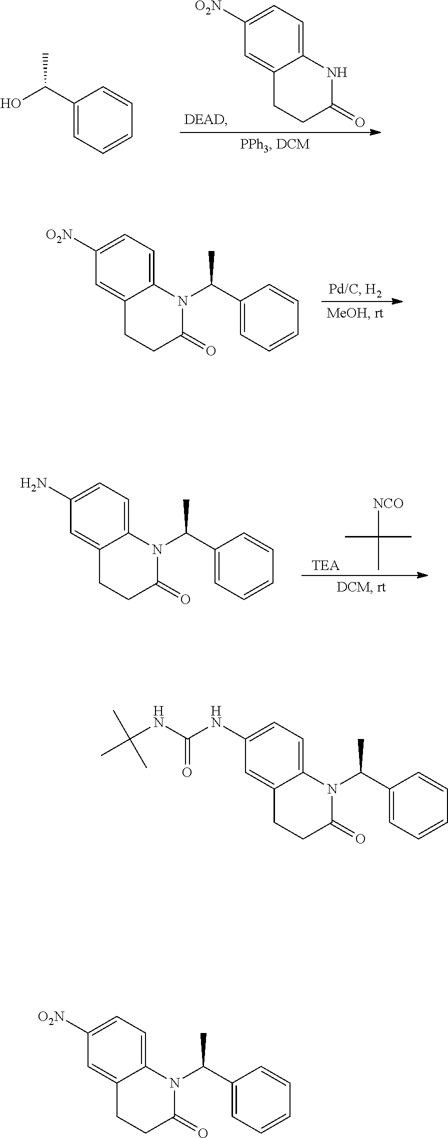

### (S)-6-nitro-1-(1-phenylethyl)-3,4-dihydroquinolin-2(1H)-one

To a solution of (R)-1-phenylethanol (1 g, 8.186 mmol, 1 equiv) in DCM (30 mL) was added 6-nitro-3,4-dihydro-1H-quinolin-2-one (1.57 g, 8.186 mmol, 1 equiv) and PPh₃ (2.15 g, 8.186 mmol, 1 equiv). DEAD (1.43 g, 8.211 mmol, 1 equiv) was added the mixture at 0° C. under a nitrogen atmosphere. The resulting mixture was stirred for 16 h at rt. The reaction mixture was quenched by water and extracted with EA (3×50 mL). The combined organic extracts were washed with brine (50 mL), dried over anhydrous Na₂SO₄, and concentrated under vacuum to yield a crude product which was directly purified by silica gel column, eluted with PE/EA (3:1) to afford (S)-6-nitro-1-(1-phenylethyl)-3,4-dihydroquinolin-2(1H)-one (300 mg, 11.61%) as a yellow solid. MS (ESI): mass calcd. for C₁₇H₁₆N₂O₃, 296.12 m/z, found 297.15 [M+H]⁺.

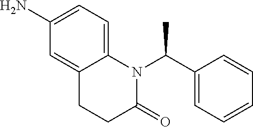 [SMILES: C[C@@H](c1ccccc1)N1C(=O)CCc2cc(N)ccc21]

### (S)-6-amino-1-(1-phenylethyl)-3,4-dihydroquinolin-2(1H)-one

To a solution of (S)-6-nitro-1-(1-phenylethyl)-3,4-dihydroquinolin-2(1H)-one (280 mg, 0.945 mmol, 1 equiv) in MeOH (10 mL) was added Pd/C (30 mg). The reaction mixture was then stirred for 1 h under hydrogen atmosphere (balloon). The reaction mixture was filtered through a pad of Celite and concentrated under reduced pressure to afford (S)-6-amino-1-(1-phenylethyl)-3,4-dihydroquinolin-2(1H)-one (220 mg, 86.81%) as a yellow solid. MS (ESI): mass calcd. for C₁₇H₁₈N₂O, 266.14 m/z, found 267.15 [M+H]⁺.

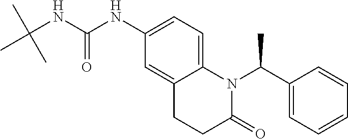 [SMILES: C[C@@H](c1ccccc1)N1C(=O)CCc2cc(NC(=O)NC(C)(C)C)ccc21]

### (S)-1-(tert-butyl)-3-(2-oxo-1-(1-phenylethyl)-1,2,3,4-tetrahydroquinolin-6-yl)urea

To a stirred solution of (S)-6-amino-1-(1-phenylethyl)-3,4-dihydroquinolin-2(1H)-one (200 mg, 0.751 mmol, 1 equiv) in DCM (10.00 mL) was added 2-isocyanato-2-methylpropane (372.2 mg, 3.755 mmol, 5 equiv) and TEA (227.9 mg, 2.253 mmol, 3 equiv) stirred overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8; Number Of Runs: 0) to afford (S)-1-(tert-butyl)-3-(2-oxo-1-(1-phenylethyl)-1,2,3,4-tetrahydroquinolin-6-yl)urea (73.9 mg, 26.88%) as a white solid. MS (ESI): mass calcd. for C₂₂H₂₇N₃O₂, 365.21 m/z, found 366.20 [M+H]⁺. ¹H NMR (300 MHz, DMSO-d₆) δ 8.07 (s, 1H), 7.18-7.39 (m, 6H), 6.81 (d, J=8.8 Hz, 1H), 6.42-6.52 (m, 1H), 6.15 (d, J=8.0 Hz, 1H), 5.91 (s, 1H), 2.80 (d, J=5.6 Hz, 2H), 2.54-2.70 (m, 2H), 1.67 (t, J=5.7 Hz, 3H), 1.24 (dd, J=4.1, 2.2 Hz, 9H).

### Example 14: (R)-1-(tert-butyl)-3-(2-oxo-1-(1-phenylethyl)-1,2,3,4-tetrahydroquinolin-6-yl)urea

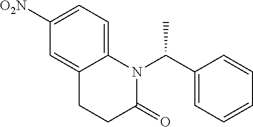 [SMILES: C[C@H](c1ccccc1)N1C(=O)CCc2cc([N+](=O)[O-])ccc21]

### (R)-6-nitro-1-(1-phenylethyl)-3,4-dihydroquinolin-2(1H)-one

To a solution of (S)-1-phenylethanol (1 g, 8.186 mmol, 1 equiv) in DCM (30 mL) was added 6-nitro-3,4-dihydro-1H-quinolin-2-one (1.57 g, 8.186 mmol, 1 equiv) and PPh₃ (2.15 g, 8.186 mmol, 1 equiv). DEAD (1.43 g, 8.211 mmol, 1 equiv) was added the mixture at 0° C. under a nitrogen atmosphere. The resulting mixture was stirred for 16 h at rt. The reaction mixture was quenched by water and extracted with EA (3×50 mL). The combined organic extracts were washed with brine (50 mL), dried over anhydrous Na₂SO₄, and concentrated under vacuum to yield a crude product which was directly purified by silica gel column, eluted with PE/EA (3:1) to afford (R)-6-nitro-1-(1-phenylethyl)-3,4-dihydroquinolin-2(1H)-one (300 mg, 11.61%) as a yellow solid. MS (ESI): mass calcd. for C₁₇H₁₆N₂O₃, 296.12 m/z, found 297.15 [M+H]⁺.

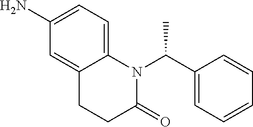 [SMILES: C[C@H](c1ccccc1)N1C(=O)CCc2cc(N)ccc21]

### (R)-6-amino-1-(1-phenylethyl)-3,4-dihydroquinolin-2(1H)-one

To a solution of (R)-6-nitro-1-(1-phenylethyl)-3,4-dihydroquinolin-2(1H)-one (280 mg, 0.945 mmol, 1 equiv) in MeOH (10 mL) was added Pd/C (30 mg). The reaction mixture was then stirred for 1 h under hydrogen atmosphere (balloon). The reaction mixture was filtered through a pad of Celite and concentrated under reduced pressure to afford (R)-6-amino-1-(1-phenylethyl)-3,4-dihydroquinolin-2(1H)-one (220 mg, 86.81%) as a yellow solid. MS (ESI): mass calcd. for C₁₇H₁₈N₂O, 266.14 m/z, found 267.15 [M+H]⁺.

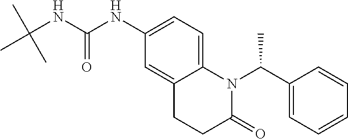 [SMILES: C[C@H](c1ccccc1)N1C(=O)CCc2cc(NC(=O)NC(C)(C)C)ccc21]

### (R)-1-(tert-butyl)-3-(2-oxo-1-(1-phenylethyl)-1,2,3,4-tetrahydroquinolin-6-yl)urea

To a stirred solution of (R)-6-amino-1-(1-phenylethyl)-3,4-dihydroquinolin-2(1H)-one (200 mg, 0.751 mmol, 1 equiv) in DCM (10.00 mL) was added 2-isocyanato-2-methylpropane (372.2 mg, 3.755 mmol, 5 equiv) and TEA (227.9 mg, 2.253 mmol, 3 equiv) stirred overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 3-tert-butyl-1-{2-oxo-1-[(1R)-1-phenylethyl]-3,4-dihydroquinolin-6-yl}urea (70 mg, 25.46%) as a white solid. MS (ESI): mass calcd. for C₂₂H₂₇N₃O₂, 365.21 m/z, found 366.20 [M+H]⁺. ¹H NMR (300 MHz, DMSO-d6) δ 8.07 (s, 1H), 7.18-7.39 (m, 6H), 6.81 (d, J=8.8 Hz, 1H), 6.42-6.52 (m, 1H), 6.15 (d, J=8.0 Hz, 1H), 5.91 (s, 1H), 2.80 (d, J=5.6 Hz, 2H), 2.54-2.70 (m, 2H), 1.67 (t, J=5.7 Hz, 3H), 1.24 (dd, J=4.1, 2.2 Hz, 9H).

### Example 15: 1-(1-benzyl-4-methyl-2-oxo-1,2,3,4-tetrahydroquinolin-6-yl)-3-(tert-butyl)urea

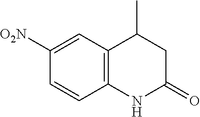

### 4-methyl-6-nitro-3,4-dihydroquinolin-2(1H)-one

To a solution of 4-methyl-3,4-dihydroquinolin-2(1H)-one (0.4 g, 2.48 mmol, 1 equiv) in H₂O (2 mL) and H₂SO₄ (9 mL) was added HNO₃ (0.3 mL) at −10° C. The reaction was stirred for 2 h at −10-0° C. The reaction mixture was quenched by water. The mixture was acidified pH=7 with NaHCO₃ (aq.) and then extracted with EA (3×50 mL). The combined organic extracts were washed with brine (50 mL), dried over anhydrous Na₂SO₄, and concentrated under vacuum to yield a crude product which was directly purified by silica gel column, eluted with PE/EA (3:1) to afford 4-methyl-6-nitro-3,4-dihydroquinolin-2(1H)-one (0.28 g, 54.79%) as a white solid. MS (ESI): mass calcd. for C₁₀H₁₀N₂O₃, 206.07 m/z, found 207.05 [M+H]⁺.

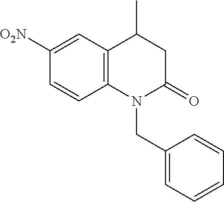 [SMILES: CC1CC(=O)N(Cc2ccccc2)c2ccc([N+](=O)[O-])cc21]

### 1-benzyl-4-methyl-6-nitro-3,4-dihydroquinolin-2(1H)-one

To a solution of 4-methyl-6-nitro-3,4-dihydroquinolin-2(1H)-one (260 mg, 1.260 mmol, 1 equiv) in DMF (10 mL) was added K₂CO₃ (522.0 mg, 3.782 mmol, 3 equiv) and (bromomethyl)benzene (237.1 mg, 1.386 mmol, 1.1 equiv). The resulting mixture was stirred for 16 h at rt. The mixture was diluted with EtOAc (50 mL), washed with water (3×30 mL), brine (2×30 mL) and concentrated under vacuum to yield a crude product which was directly purified by silica gel column, eluted with PE/EA (3:1) to afford 1-benzyl-4-methyl-6-nitro-3,4-dihydroquinolin-2(1H)-one (200 mg, 53.62%) as a white solid. MS (ESI): mass calcd. for C₁₇H₁₆N₂O₃: 296.12 m/z, found 297.05 [M+H]⁺.

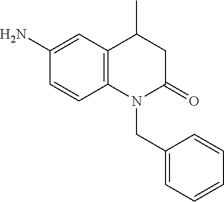 [SMILES: CC1CC(=O)N(Cc2ccccc2)c2ccc(N)cc21]

### 6-amino-1-benzyl-4-methyl-3,4-dihydroquinolin-2(1H)-one

To a solution of 1-benzyl-4-methyl-6-nitro-3,4-dihydroquinolin-2(1H)-one (180 mg, 0.607 mmol, 1 equiv) in EtOH (10 mL) and H₂O (2.5 mL) was added Fe (339.3 mg, 6.07 mmol, 10 equiv) and NH₄Cl (324.7 mg, 6.07 mmol, 10 equiv). The resulting mixture was stirred for 2 h at 60° C. and stirred until the starting material was totally consumed by TLC. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 6-amino-1-benzyl-4-methyl-3,4-dihydroquinolin-2(1H)-one (120 mg, 74.07%) as a yellow solid. MS (ESI): mass calcd. for C₁₇H₁₈N₂O: 266.14 m/z, found 267.15 [M+H]⁺.

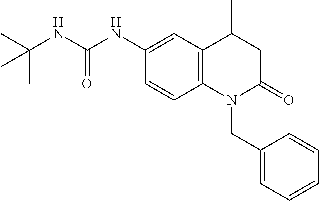 [SMILES: CC1CC(=O)N(Cc2ccccc2)c2ccc(NC(=O)NC(C)(C)C)cc21]

### 1-(1-benzyl-4-methyl-2-oxo-1,2,3,4-tetrahydroquinolin-6-yl)-3-(tert-butyl)urea

To a solution of 6-amino-1-benzyl-4-methyl-3,4-dihydroquinolin-2(1H)-one (120 mg, 0.451 mmol, 1 equiv) in DCM (5 mL) was added 2-isocyanato-2-methylpropane (133.9 mg, 1.351 mmol, 3 equiv) and TEA (136.4 mg, 1.351 mmol, 3 equiv). The resulting mixture was stirred for overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column, eluted with PE/EA (2:1) & Prep-HPLC (Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 1-(1-benzyl-4-methyl-2-oxo-1,2,3,4-tetrahydroquinolin-6-yl)-3-(tert-butyl)urea (47.9 mg, 44.35%) as a white solid. MS (ESI): mass calcd. for C₂₂H₂₇N₃O₂: 365.21 m/z, found 366.15 [M+H]⁺. ¹H NMR (300 MHz, DMSO-d6) δ 8.12 (s, 1H), 7.21-7.32 (m, 6H), 6.99 (dd, J=8.8, 2.4 Hz, 1H), 6.82 (d, J=8.8 Hz, 1H), 5.90 (s, 1H), 5.05-5.17 (m, 2H), 2.90-3.13 (m, 1H), 2.68-2.86 (m, 1H), 2.33-2.50 (m, 1H), 1.26 (s, 9H), 1.18 (d, J=6.9 Hz, 3H).

### Example 16: 1-(1-benzyl-2-oxo-1,2,3,4-tetrahydroquinolin-6-yl)-3-(4-hydroxy-2-methylbutan-2-yl)urea

### Synthetic Scheme

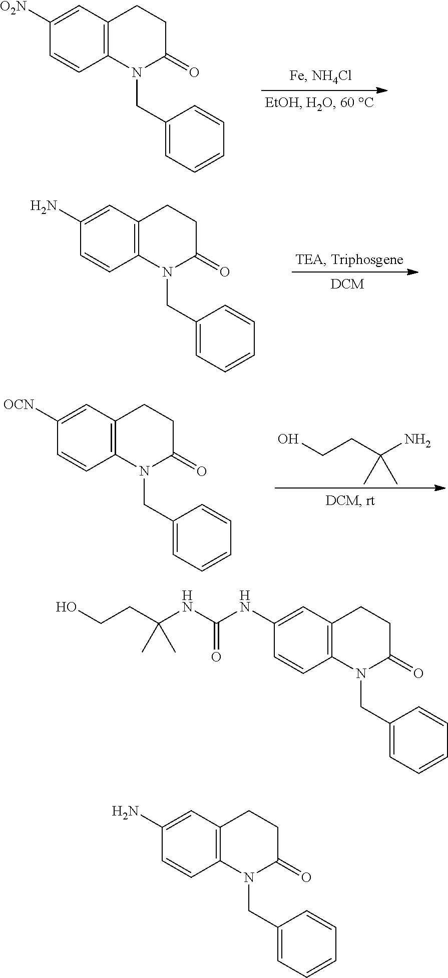

### 6-amino-1-benzyl-3,4-dihydroquinolin-2(1H)-one

A solution of 1-benzyl-6-nitro-3,4-dihydroquinolin-2-one (400 mg, 1.417 mmol, 1 equiv) in EtOH (10 mL) and H₂O (2 mL) was treated with Fe (791.29 mg, 14.170 mmol, 10 equiv) and NH₄Cl (757.92 mg, 14.170 mmol, 10 equiv) for 2 h at 60° C. The resulting mixture was concentrated under reduced pressure. The residue was purified by silica gel column chromatography, eluted with DCM/MeOH (5%-11%) to afford 6-amino-1-benzyl-3,4-dihydroquinolin-2-one (320 mg, 89.51%) as a white solid. MS (ESI): mass calcd. for C₁₆H₁₆N₂O, 252.13m/z, found 253.25 [M+H]⁺.

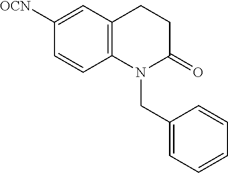

### 1-benzyl-6-isocyanato-3,4-dihydroquinolin-2(1H)-one

To a stirred solution of 6-amino-1-benzyl-3,4-dihydroquinolin-2-one (180 mg, 0.713 mmol, 1 equiv) in anhydrous DCM (5 mL) was added TEA (216.57 mg, 2.139 mmol, 3 equiv) and Triphosgene (105.84 mg, 0.356 mmol, 0.5 equiv) at 0° C., and stirred for 2 h at rt. After completion of reaction to afford 1-benzyl-6-isocyanato-3,4-dihydroquinolin-2-one as a crude. MS (ESI): mass calcd. for C₁₇H₁₄N₂O₂, 278.11m/z, found 279.05 [M+H]⁺

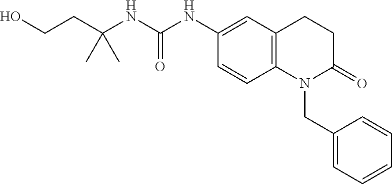 [SMILES: CC(C)(CCO)NC(=O)Nc1ccc2c(c1)CCC(=O)N2Cc1ccccc1]

### 1-(1-benzyl-2-oxo-1,2,3,4-tetrahydroquinolin-6-yl)-3-(4-hydroxy-2-methylbutan-2-yl)urea

To a stirred solution of 1-benzyl-6-isocyanato-3,4-dihydroquinolin-2-one (crude) in anhydrous DCM (5 mL) was added 3-amino-3-methylbutan-1-ol (220.3 mg, 2.139 mmol, 3 equiv) was added at 0° C. The resulting mixture was stirred for 1 h at rt. Desired product could be detected by LCMS. The resulting mixture was concentrated under reduced pressure. The crude product was purified by HPLC with the following conditions Column: XBridge Shield RP18 OBD Column, 30*150 mm, 5 μm; Mobile Phase A: Water(10 mmol/L NH₄HCO₃), Mobile Phase B: ACN; Flow rate: 60 mL/min; Gradient: 21% B to 41% B in 10 min, 41% B; Wave Length: 254/220 nm; RT1(min): 10; Number Of Runs: 0; to afford 1-(1-benzyl-2-oxo-3,4-dihydroquinolin-6-yl)-3-(4-hydroxy-2-methylbutan-2-yl)urea (23.3 mg, 16.47%) as a white solid. MS (ESI): mass calcd. for C₂₂H₂₇N₃O₃: 381.21 m/z, found 382.15 [M+H]⁺. ¹H NMR (300 MHz, DMSO-d₆) δ 8.18 (s, 1H), 7.39-7.12 (m, 6H), 6.96 (dd, J=8.8, 2.5 Hz, 1H), 6.77 (d, J=8.8 Hz, 1H), 5.93 (s, 1H), 5.10 (s, 2H), 4.39 (t, J=4.9 Hz, 1H), 3.48 (td, J=7.1, 4.7 Hz, 2H), 2.88 (dd, J=8.8, 5.8 Hz, 2H), 2.65 (dd, J=8.8, 5.8 Hz, 2H), 1.79 (t, J=7.2 Hz, 2H), 1.25 (s, 6H).

### Example 17: 1-(tert-butyl)-3-(1-(4-cyanobenzyl)-2-oxo-1,2,3,4-tetrahydroquinolin-6-yl)urea

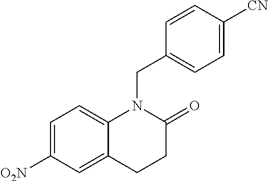 [SMILES: N#Cc1ccc(CN2C(=O)CCc3cc([N+](=O)[O-])ccc32)cc1]

### 4-((6-nitro-2-oxo-3,4-dihydroquinolin-1(2H)-yl)methyl)benzonitrile

To a solution of 6-nitro-3,4-dihydroquinolin-2(1H)-one (500 mg, 2.602 mmol, 1 equiv) in DMF (10 mL) was added K₂CO₃ (1086.6 mg, 7.805 mmol, 3 equiv) and 4-(bromomethyl)benzonitrile (558.09 mg, 2.862 mmol, 1.1 equiv). The resulting mixture was stirred for 16 h at rt. The mixture was diluted with EtOAc (50 mL), washed with water (3×30 mL), brine (2×30 mL) and concentrated under vacuum to yield a crude product which was directly purified by silica gel column, eluted with PE/EA (3:1) to afford 4-((6-nitro-2-oxo-3,4-dihydroquinolin-1(2H)-yl)methyl)benzonitrile (400 mg, 50.00%) as a white solid. MS (ESI): mass calcd. for C₁₇H₁₃N₃O₃: 307.10 m/z, found 308.15 [M+H]⁺.

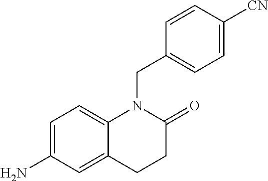 [SMILES: N#Cc1ccc(CN2C(=O)CCc3cc(N)ccc32)cc1]

### 4-((6-amino-2-oxo-3,4-dihydroquinolin-1(2H)-yl)methyl)benzonitrile

To a solution of 4-((6-nitro-2-oxo-3,4-dihydroquinolin-1(2H)-yl)methyl)benzonitrile (300 mg, 0.976 mmol, 1 equiv) in EtOH (10 mL) and H₂O (2.5 mL) was added Fe (545.1 mg, 9.76 mmol, 10 equiv) and NH₄Cl (522.1 mg, 9.76 mmol, 10 equiv). The resulting mixture was stirred for 2 h at 60° C. and stirred until the starting material was totally consumed by TLC. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 4-((6-amino-2-oxo-3,4-dihydroquinolin-1(2H)-yl)methyl)benzonitrile (108 mg, 40.05%) as a yellow solid. MS (ESI): mass calcd. for C₁₇H₁₅N₃O: 277.12 m/z, found 278.25 [M+H]⁺.

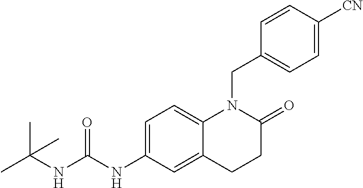 [SMILES: CC(C)(C)NC(=O)Nc1ccc2c(c1)CCC(=O)N2Cc1ccc(C#N)cc1]

### 1-(tert-butyl)-3-(1-(4-cyanobenzyl)-2-oxo-1,2,3,4-tetrahydroquinolin-6-yl)urea

To a solution of 4-((6-amino-2-oxo-3,4-dihydroquinolin-1(2H)-yl)methyl)benzonitrile (80 mg, 0.288 mmol, 1 equiv) in DCM (5 mL) was added 2-isocyanato-2-methylpropane (85.8 mg, 0.865 mmol, 3 equiv) and TEA (87.4 mg, 0.865 mmol, 3 equiv). The resulting mixture was stirred for overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column, eluted with PE/EA (2:1) & Prep-HPLC (Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 25% B to 45% B in 10.5 min; Wave Length: 220 nm; RT1(min): 12; Number Of Runs: 1) to afford 1-(tert-butyl)-3-(1-(4-cyanobenzyl)-2-oxo-1,2,3,4-tetrahydroquinolin-6-yl)urea (38.9 mg, 36.02%) as a white solid. MS (ESI): mass calcd. for C₂₂H₂₄N₄O: 376.19 m/z, found 377.10 [M+H]⁺. ¹H NMR (400 MHz, DMSO-d6) δ 8.12 (s, 1H), 7.75-7.81 (m, 2H), 7.40 (d, J=8.2 Hz, 2H), 7.34 (d, J=2.4 Hz, 1H), 6.97 (dd, J=8.8, 2.5 Hz, 1H), 6.71 (d, J=8.8 Hz, 1H), 5.92 (s, 1H), 5.18 (s, 2H), 2.86-2.94 (m, 2H), 2.63-2.71 (m, 2H), 1.26 (s, 9H).

### Example 18: 1-(tert-butyl)-3-(1-(3-cyanobenzyl)-2-oxo-1,2,3,4-tetrahydroquinolin-6-yl)urea

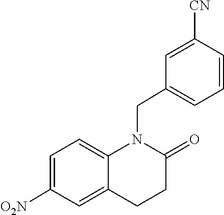 [SMILES: N#Cc1cccc(CN2C(=O)CCc3cc([N+](=O)[O-])ccc32)c1]

### 3-((6-nitro-2-oxo-3,4-dihydroquinolin-1(2H)-yl)methyl)benzonitrile

To a solution of 6-nitro-3,4-dihydroquinolin-2(1H)-one (500 mg, 2.602 mmol, 1 equiv) in DMF (10 mL) was added K₂CO₃ (1086.6 mg, 7.805 mmol, 3 equiv) and 3-(bromomethyl)benzonitrile (558.09 mg, 2.862 mmol, 1.1 equiv). The resulting mixture was stirred for 16 h at rt. The mixture was diluted with EtOAc (50 mL), washed with water (3×30 mL), brine (2×30 mL) and concentrated under vacuum to yield a crude product which was directly purified by silica gel column, eluted with PE/EA (3:1) to afford 3-((6-nitro-2-oxo-3,4-dihydroquinolin-1(2H)-yl)methyl)benzonitrile (350 mg, 43.75%) as a white solid. MS (ESI): mass calcd. for C₁₇H₁₃N₃O₃: 307.10 m/z, found 308.05 [M+H]⁺.

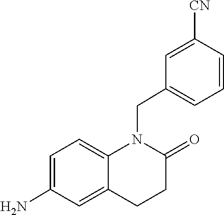 [SMILES: N#Cc1cccc(CN2C(=O)CCc3cc(N)ccc32)c1]

### 3-((6-amino-2-oxo-3,4-dihydroquinolin-1(2H)-yl)methyl)benzonitrile

To a solution of 3-((6-nitro-2-oxo-3,4-dihydroquinolin-1(2H)-yl)methyl)benzonitrile (300 mg, 0.976 mmol, 1 equiv) in EtOH (10 mL) and H₂O (2.5 mL) was added Fe (545.1 mg, 9.76 mmol, 10 equiv) and NH₄Cl (522.1 mg, 9.76 mmol, 10 equiv). The resulting mixture was stirred for 2 h at 60° C. and stirred until the starting material was totally consumed by TLC. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 3-((6-amino-2-oxo-3,4-dihydroquinolin-1(2H)-yl)methyl)benzonitrile (120 mg, 44.44%) as a yellow solid. MS (ESI): mass calcd. for C₁₇H₁₅N₃O: 277.12 m/z, found 278.20 [M+H]⁺.

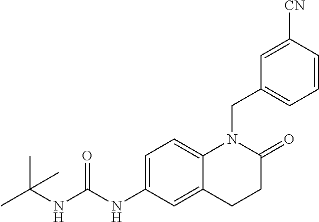 [SMILES: CC(C)(C)NC(=O)Nc1ccc2c(c1)CCC(=O)N2Cc1cccc(C#N)c1]

### 1-(tert-butyl)-3-(1-(3-cyanobenzyl)-2-oxo-1,2,3,4-tetrahydroquinolin-6-yl)urea

To a solution of 3-((6-amino-2-oxo-3,4-dihydroquinolin-1(2H)-yl)methyl)benzonitrile (80 mg, 0.288 mmol, 1 equiv) in DCM (5 mL) was added 2-isocyanato-2-methylpropane (85.8 mg, 0.865 mmol, 3 equiv) and TEA (87.4 mg, 0.865 mmol, 3 equiv). The resulting mixture was stirred for overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column, eluted with PE/EA (2:1) & Prep-HPLC (Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 25% B to 45% B in 10.5 min; Wave Length: 220 nm; RT1(min): 12; Number Of Runs: 1) to afford 1-(tert-butyl)-3-(1-(3-cyanobenzyl)-2-oxo-1,2,3,4-tetrahydroquinolin-6-yl)urea (47.9 mg, 44.35%) as a white solid. MS (ESI): mass calcd. for C₂₂H₂₄N₄O: 376.19 m/z, found 377.10 [M+H]⁺. ¹H NMR (400 MHz, DMSO-d6) δ 8.12 (s, 1H), 7.67-7.75 (m, 2H), 7.48-7.58 (m, 2H), 7.33 (d, J=2.4 Hz, 1H), 6.98 (dd, J=8.8, 2.5 Hz, 1H), 6.74 (d, J=8.8 Hz, 1H), 5.92 (s, 1H), 5.14 (s, 2H), 2.86-2.94 (m, 2H), 2.64-2.72 (m, 2H), 1.26 (s, 9H).

### Example 1: 3-tert-butyl-1-{1-[(2-fluorophenyl)methyl]-2-oxo-3,4-dihydroquinolin-6-yl}urea

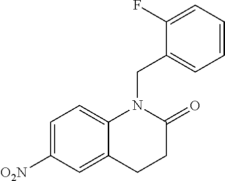 [SMILES: O=C1CCc2cc([N+](=O)[O-])ccc2N1Cc1ccccc1F]

### 1-[(2-fluorophenyl)methyl]-6-nitro-3,4-dihydroquinolin-2-one

To a solution of 6-nitro-3,4-dihydro-1H-quinolin-2-one (2 g, 10.407 mmol, 1 equiv) and 1-(bromomethyl)-2-fluorobenzene (3147.60 mg, 16.651 mmol, 1.6 equiv) in dimethylformamide (20 mL) was added K₂CO₃ (4346.47 mg, 31.221 mmol, 3 equiv). The reaction was stirred at rt for 3h. Quenched with water (100 mL) and extracted with EA (3×100 mL). The combined extracts were washed with water, brine, dried over Na₂SO₄, filtered, and concentrated. The residue was purified by column chromatography on silica gel with EA/PE (0-100%) to give 1-[(2-fluorophenyl)methyl]-6-nitro-3,4-dihydroquinolin-2-one (2.06 g, 65.92%) as a white solid. MS (ESI): mass calcd. for C₁₆H₁₃FN₂O₃: 300.09 m/z, found 301.05[M+H]⁺.

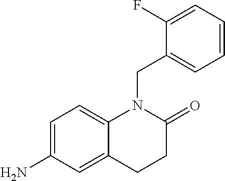 [SMILES: Nc1ccc2c(c1)CCC(=O)N2Cc1ccccc1F]

### 6-amino-1-[(2-fluorophenyl)methyl]-3,4-dihydroquinolin-2-one

To a solution of 1-[(2-fluorophenyl)methyl]-6-nitro-3,4-dihydroquinolin-2-one (1 g, 3.330 mmol, 1 equiv) in EA (35 mL) was added Pd/C (1 g). The resulting mixture was stirred for 1 h at room temperature under hydrogen atmosphere. Desired product could be detected by LCMS. The resulting mixture was filtered, the filter cake was washed with EA (3×10 mL). The filtrate was concentrated under reduced pressure. The residue was purified by column chromatography on silica gel with EA/PE (0-100%) to give 6-amino-1-[(2-fluorophenyl)methyl]-3,4-dihydroquinolin-2-one (990 mg, 109.98%) as a white solid. MS (ESI): mass calcd for C₁₆H₁₅FN₂O: 270.12 m/z, found 271.10[M+H]⁺.

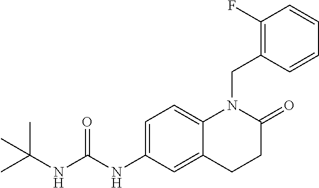 [SMILES: CC(C)(C)NC(=O)Nc1ccc2c(c1)CCC(=O)N2Cc1ccccc1F]

### 3-tert-butyl-1-{1-[(2-fluorophenyl)methyl]-2-oxo-3,4-dihydroquinolin-6-yl}urea

To a solution of 6-amino-1-[(2-fluorophenyl)methyl]-3,4-dihydroquinolin-2-one (980 mg, 3.626 mmol, 1 equiv) in DCM/toluene (10 mL, 2/1) was added 2-isocyanato-2-methylpropane (1077.9 mg, 10.873 mmol, 3.00 equiv) The reaction was stirred at 90° C. for 1 h. Quenched with water (50 mL) and extracted with EA (3×50 mL). The combined extracts were washed with water, brine, dried over Na₂SO₄, filtered, and concentrated. The residue was purified by preparative HPLC using aXBridge Prep OBD C18 Column 150 mm×30 mm×5 μm column (eluent: 17% to 42% (v/v) CH₃CN and H₂O with 10 mmol/L NH4HCO3) to afford the title compound 3-tert-butyl-1-{1-[(2-fluorophenyl)methyl]-2-oxo-3,4-dihydroquinolin-6-yl}urea (412.3 mg, 30.33%) as a white solid. LC/MS (ESI): mass calcd. for C₂₁H₂₄FN₃O₂:369.19 m/z, found:370.05 [M+H]⁺. ¹H NMR (400 MHz, DMSO-d6) δ 8.13 (s, 1H), 7.35 (m, 1H), 7.23-7.32 (m, 2H), 7.00-7.09 (m, 3H), 6.73 (m, 1H), 5.93 (s, 1H), 5.12 (s, 2H), 2.89 (m, 2H), 2.66 (m, 2H), 1.27 (s, 9H). ¹⁹F NMR (376 MHz, DMSO) δ−118.31.

### Example 20: 1-(tert-butyl)-3-(1-(2-cyanobenzyl)-2-oxo-1,2,3,4-tetrahydroquinolin-6-yl)urea

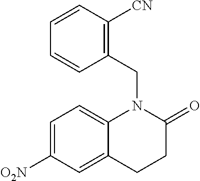 [SMILES: N#Cc1ccccc1CN1C(=O)CCc2cc([N+](=O)[O-])ccc21]

### 2-((6-nitro-2-oxo-3,4-dihydroquinolin-1(2H)-yl)methyl)benzonitrile

To a stirred solution of 6-nitro-3,4-dihydro-1H-quinolin-2-one (400 mg, 2.081 mmol, 1 equiv) in DMF (10 mL) was added 2-(bromomethyl)benzonitrile (612.09 mg, 3.122 mmol, 1.5 equiv) and potassium methaneperoxoate potassium (869.29 mg, 6.243 mmol, 3 equiv) at rt and stirred for overnight. The reaction progress was monitored by LCMS. The resulting mixture was extracted with brine (1×30 mL). The combined organic layers were washed with EA (3×30 mL), dried over anhydrous Na₂SO₄. After filtration, the filtrate was concentrated under reduced pressure. The residue was purified by silica gel column chromatography, eluted with PE/EA (3:1) to afford 2-[(6-nitro-2-oxo-3,4-dihydroquinolin-1-yl)methyl]benzonitrile (447 mg, 70.0%) as a yellow solid. MS (ESI): mass calcd. for C₁₇H₁₃N₃O₃: 307.10 m/z, found 308.10 [M+H]⁺.

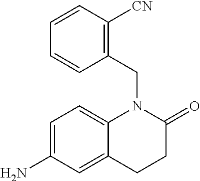 [SMILES: N#Cc1ccccc1CN1C(=O)CCc2cc(N)ccc21]

### 2-((6-amino-2-oxo-3,4-dihydroquinolin-1(2H)-yl)methyl)benzonitrile

To a stirred solution of 2-[(6-nitro-2-oxo-3,4-dihydroquinolin-1-yl)methyl]benzonitrile (400 mg, 1.302 mmol, 1 equiv) in ethyl alcohol (10 mL) and water (2 mL) was added iron (726.89 mg, 13.020 mmol, 10 equiv) and NH₄Cl (696.24 mg, 13.020 mmol, 10 equiv) and stirred for 1 h at 60° C. The resulting mixture was concentrated under reduced pressure. The residue was purified by silica gel column chromatography eluted with PE/EA (2:1) to afford 2-((6-amino-2-oxo-3,4-dihydroquinolin-1(2H)-yl)methyl)benzonitrile (280 mg, 77.57%) as a white solid. MS (ESI): mass calcd. for C₁₇H₁₅N₃O: 277.12 m/z, found 278.05 [M+H]⁺.

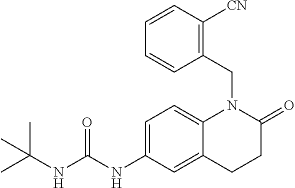 [SMILES: CC(C)(C)NC(=O)Nc1ccc2c(c1)CCC(=O)N2Cc1ccccc1C#N]

### 1-(tert-butyl)-3-(1-(2-cyanobenzyl)-2-oxo-1,2,3,4-tetrahydroquinolin-6-yl)urea

A solution of 2-((6-amino-2-oxo-3,4-dihydroquinolin-1(2H)-yl)methyl)benzonitrile (150 mg, 0.541 mmol, 1 equiv) in DCM (5 mL) was add TEA (164.20 mg, 1.623 mmol, 3 equiv) and 2-isocyanato-2-methylpropane (214.48 mg, 2.164 mmol, 4 equiv) at 0° C. The resulting mixture was stirred for 1 h at rt. The resulting mixture was concentrated under reduced pressure. The crude product was purified by HPLC with the following conditions (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0; to afford 3-tert-butyl-1-{1-[(2-cyanophenyl)methyl]-2-oxo-3,4-dihydroquinolin-6-yl}urea (69.2 mg, 33.91%) as a white solid. MS (ESI):mass calcd. for C₂₂H₂₄N₄O₂: 376.19 m/z, found 377.15 [M+H]⁺. ¹H NMR (300 MHz, DMSO-d₆) δ 8.15 (s, 1H), 7.86 (dd, J=7.7, 1.3 Hz, 1H), 7.61 (td, J=7.7, 1.4 Hz, 1H), 7.44 (td, J=7.6, 1.2 Hz, 1H), 7.37 (d, J=2.4 Hz, 1H), 7.25-7.18 (m, 1H), 7.00 (dd, J=8.7, 2.5 Hz, 1H), 6.69 (d, J=8.8 Hz, 1H), 5.94 (s, 1H), 5.25 (s, 2H), 2.93 (dd, J=8.7, 5.8 Hz, 2H), 2.67 (dd, J=8.7, 5.8 Hz, 2H), 1.27 (s, 9H).

### Example 21: 1-(tert-butyl)-3-(1-(3-methylbenzyl)-2-oxo-1,2,3,4-tetrahydroquinolin-6-yl)urea

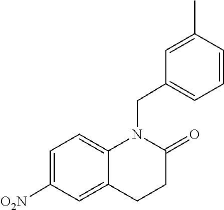 [SMILES: Cc1cccc(CN2C(=O)CCc3cc([N+](=O)[O-])ccc32)c1]

### 6-amino-1-[(3-methylphenyl)methyl]-3,4-dihydroquinolin-2-one

To a solution of 6-nitro-3,4-dihydro-1H-quinolin-2-one (500 mg, 2.602 mmol, 1 equiv) in DMF (8 mL) was added K₂CO₃ (1086.6 mg, 7.862 mmol, 3 equiv) and 1-(bromomethyl)-3-methylbenzene (529.6 mg, 2.862 mmol, 1.1 equiv). The resulting mixture was stirred for 16 h at rt. The mixture was diluted with EtOAc (100 mL), washed with water (3×50 mL), brine (2×50 mL) and concentrated under vacuum to yield a crude product which was directly purified by silica gel column, eluted with PE/EA (3:1) to afford 1-[(3-methylphenyl)methyl]-6-nitro-3,4-dihydroquinolin-2-one (500 mg, 64.44%) as a yellow solid. MS (ESI): mass calcd. for C₁₇H₁₆N₂O₃ 296.12 m/z, found 297.00 [M+H]⁺.

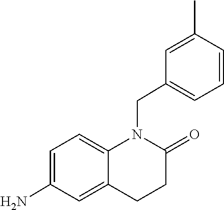 [SMILES: Cc1cccc(CN2C(=O)CCc3cc(N)ccc32)c1]

### 6-amino-1-(1-phenylpropyl)-3,4-dihydroquinolin-2-one

To a solution of 1-[(3-methylphenyl)methyl]-6-nitro-3,4-dihydroquinolin-2-one (500 mg, 1.687 mmol, 1 equiv) in EtOH (10 mL) and H₂O (2.5 mL) was added Fe (942.2 mg, 16.873 mmol, 10 equiv) and NH₄Cl (902.5 mg, 16.873 mmol, 10 equiv). The resulting mixture was stirred for 2 h at 60° C. The mixture was diluted with EtOAc (100 mL), washed with water (3×30 mL), brine (2×30 mL) and concentrated under vacuum to yield a crude product which was directly purified by silica gel column, eluted with PE/EA (2:1) to afford 6-amino-1-[(3-methylphenyl)methyl]-3,4-dihydroquinolin-2-one (400 mg, 88.46%) as a yellow solid. MS (ESI): mass calcd. for C₁₇H₁₈N₂O 266.14 m/z, found 267.10 [M+H]⁺.

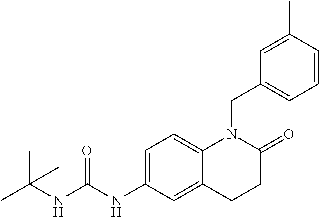 [SMILES: Cc1cccc(CN2C(=O)CCc3cc(NC(=O)NC(C)(C)C)ccc32)c1]

### 1-(tert-butyl)-3-(1-(3-methylbenzyl)-2-oxo-1,2,3,4-tetrahydroquinolin-6-yl)urea

To a solution of 6-amino-1-[(3-methylphenyl)methyl]-3,4-dihydroquinolin-2-one (400 mg, 1.502 mmol, 1 equiv) in DCM (10 mL) was added 2-isocyanato-2-methylpropane (446.6 mg, 4.506 mmol, 3 equiv) and TEA (455.9 mg, 4.505 mmol, 3 equiv). The resulting mixture was stirred for overnight at rt. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XBridge Shield RP18 OBD Column, 30*150 mm, 5 m; Mobile Phase A: Water(10 mmol/L NH₄HCO₃), Mobile Phase B: ACN; Flow rate: 60 mL/min; Gradient: 38% B to 60% B in 10 min, 60% B; Wave Length: 254/220 nm; RT1(min): 10; Number Of Runs: 0) to afford 1-(tert-butyl)-3-(1-(3-methylbenzyl)-2-oxo-1,2,3,4-tetrahydroquinolin-6-yl)urea (282.3 mg, 51.15%) as a white solid. MS (ESI): mass calcd. for C₂₂H₂₇N₃O₂, 365.21 m/z, found 366.05 [M+H]⁺. ¹H NMR (300 MHz, DMSO-d₆) δ 8.11 (s, 1H), 7.33 (d, J=2.4 Hz, 1H), 7.17 (t, J=7.7 Hz, 1H), 6.92-7.09 (m, 4H), 6.76 (d, J=8.8 Hz, 1H), 5.92 (s, 1H), 5.05 (s, 2H), 2.87 (dd, J=8.8, 5.8 Hz, 2H), 2.64 (dd, J=8.7, 5.8 Hz, 2H), 2.25 (s, 3H), 1.27 (s, 9H).

### Example 22: 1-(tert-butyl)-3-(1-(3-methoxybenzyl)-2-oxo-1,2,3,4-tetrahydroquinolin-6-yl)urea

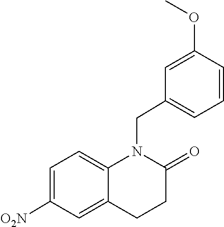 [SMILES: COc1cccc(CN2C(=O)CCc3cc([N+](=O)[O-])ccc32)c1]

### 1-[(3-methoxyphenyl)methyl]-6-nitro-3,4-dihydroquinolin-2-one

To a solution of 6-nitro-3,4-dihydro-1H-quinolin-2-one (500 mg, 2.602 mmol, 1 equiv) in DMF (8 mL) was added K₂CO₃ (1078.7 mg, 7.862 mmol, 3 equiv) and 1-(bromomethyl)-3-methoxybenzene (575.4 mg, 2.862 mmol, 1.1 equiv). The resulting mixture was stirred for 16 h at rt. The mixture was diluted with EtOAc (100 mL), washed with water (3×30 mL), brine (2×30 mL) and concentrated under vacuum to yield a crude product which was directly purified by silica gel column, eluted with PE/EA (3:1) to afford 1-[(3-methoxyphenyl)methyl]-6-nitro-3,4-dihydroquinolin-2-one (500 mg, 60.71%) as a yellow solid. MS (ESI): mass calcd. for C₁₇H₁₆N₂O₄ 312.11 m/z, found 313.05 [M+H]⁺.

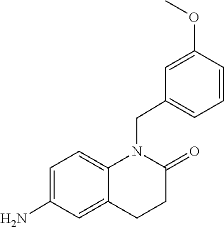 [SMILES: COc1cccc(CN2C(=O)CCc3cc(N)ccc32)c1]

### 6-amino-1-(1-phenylpropyl)-3,4-dihydroquinolin-2-one

To a solution of 1-[(3-methoxyphenyl)methyl]-6-nitro-3,4-dihydroquinolin-2-one (500 mg, 1.601 mmol, 1 equiv) in EtOH (10 mL) and H₂O (2.5 mL) was added Fe (894.0 mg, 16.009 mmol, 10 equiv) and NH₄Cl (856.3 mg, 16.009 mmol, 10 equiv). The resulting mixture was stirred for 2 h at 60° C. The mixture was diluted with EtOAc (100 mL), washed with water (3×30 mL), brine (2×30 mL) and concentrated under vacuum to yield a crude product which was directly purified by silica gel column, eluted with PE/EA (2:1) to afford 6-amino-1-[(3-methoxyphenyl)methyl]-3,4-dihydroquinolin-2-one (400 mg, 88.33%) as a yellow solid. MS (ESI): mass calcd. for C₁₇H₁₈N₂O₂ 282.14 m/z, found 283.10 [M+H]⁺.

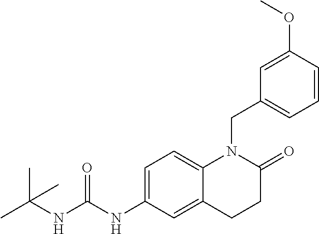 [SMILES: COc1cccc(CN2C(=O)CCc3cc(NC(=O)NC(C)(C)C)ccc32)c1]

### 1-(tert-butyl)-3-(1-(3-methoxybenzyl)-2-oxo-1,2,3,4-tetrahydroquinolin-6-yl)urea

To a solution of 6-amino-1-[(3-methoxyphenyl)methyl]-3,4-dihydroquinolin-2-one (400 mg, 1.417 mmol, 1 equiv) in DCM (10 mL) was added 2-isocyanato-2-methylpropane (421.3 mg, 4.250 mmol, 3 equiv) and TEA (430.0 mg, 4.250 mmol, 3 equiv). The resulting mixture was stirred for overnight at rt. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 26% B to 50% B in 9 min, 50% B; Wave Length: 254/220 nm; RT1(min): 8.6; Number Of Runs: 0) to afford 1-(tert-butyl)-3-(1-(3-methoxybenzyl)-2-oxo-1,2,3,4-tetrahydroquinolin-6-yl)urea (85.3 mg, 15.75%) as a white solid. MS (ESI): mass calcd. for C₂₂H₂₇N₃O₃, 381.21 m/z, found 382.15 [M+H]⁺. ¹H NMR (300 MHz, DMSO-d6) δ 8.11 (s, 1H), 7.33 (d, J=2.4 Hz, 1H), 7.16-7.25 (m, 1H), 6.97 (dd, J=8.7, 2.5 Hz, 1H), 6.71-6.82 (m, 4H), 5.92 (s, 1H), 5.06 (s, 2H), 3.70 (s, 3H), 2.87 (dd, J=8.8, 5.8 Hz, 2H), 2.64 (dd, J=8.7, 5.8 Hz, 2H), 1.26 (s, 9H).

### Example 23: 1-(tert-butyl)-3-(2-oxo-1-(3-(trifluoromethyl)benzyl)-1,2,3,4-tetrahydroquinolin-6-yl)urea

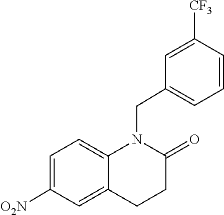 [SMILES: O=C1CCc2cc([N+](=O)[O-])ccc2N1Cc1cccc(C(F)(F)F)c1]

### 6-nitro-1-(3-(trifluoromethyl)benzyl)-3,4-dihydroquinolin-2(1H)-one

To a solution of 6-nitro-3,4-dihydro-1H-quinolin-2-one (500 mg, 2.602 mmol, 1 equiv) in DMF (8 mL) was added K₂CO₃ (1086.6 mg, 7.862 mmol, 3 equiv) and 1-(bromomethyl)-3-(trifluoromethyl)benzene (684.1 mg, 2.862 mmol, 1.1 equiv). The resulting mixture was placed at rt and stirred overnight until the starting material was totally consumed by LCMS, The mixture was diluted with EtOAc (100 mL), washed with water (3×30 mL), brine (2×30 mL) and concentrated under vacuum to yield a crude product which was directly purified by flash chromatography (PE/EA=3:1r) to afford 6-nitro-1-{[3-(trifluoromethyl)phenyl]methyl}-3,4-dihydroquinolin-2-one (500 mg, 54.86%) as a yellow solid. MS (ESI): mass calcd. for C₁₇H₁₃F₃N₂O₃ 350.09 m/z, found 350.95 [M+H]⁺.

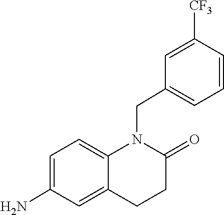 [SMILES: Nc1ccc2c(c1)CCC(=O)N2Cc1cccc(C(F)(F)F)c1]

### 6-amino-1-(3-(trifluoromethyl)benzyl)-3,4-dihydroquinolin-2(1H)-one

To a solution of 6-nitro-1-{[3-(trifluoromethyl)phenyl]methyl}-3,4-dihydroquinolin-2-one (500 mg, 1.427 mmol, 1 equiv) in EtOH (10 mL) and H₂O (2.5 mL) was added Fe (797.1 mg, 14.274 mmol, 10 equiv) and NH₄Cl (763.4 mg, 14.274 mmol, 10 equiv). The resulting mixture was stirred 30 min at 60° C. until the starting material was totally consumed by LCMS, The mixture was diluted with EtOAc (100 mL), washed with water (3×30 mL), brine (2×30 mL) and concentrated under vacuum to yield a crude product which was directly purified by flash chromatography (PE/EA=2:1) to afford 6-amino-1-{[3-(trifluoromethyl)phenyl]methyl}-3,4-dihydroquinolin-2-one (300 mg, 64.88%) as a yellow solid. MS (ESI): mass calcd. for C₁₇H₁₅F₃N₂O 320.11 m/z, found 321.05 [M+H]⁺.

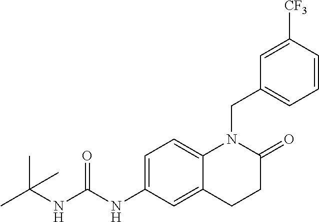 [SMILES: CC(C)(C)NC(=O)Nc1ccc2c(c1)CCC(=O)N2Cc1cccc(C(F)(F)F)c1]

### 1-(tert-butyl)-3-(2-oxo-1-(3-(trifluoromethyl)benzyl)-1,2,3,4-tetrahydroquinolin-6-yl)urea

To a solution of 6-amino-1-{[3-(trifluoromethyl)phenyl]methyl}-3,4-dihydroquinolin-2-one (300 mg, 0.937 mmol, 1 equiv) in DCM (10 mL) was added 2-isocyanato-2-methylpropane (278.5 mg, 2.810 mmol, 3 equiv) and TEA (284.3 mg, 2.810 mmol, 3 equiv). The resulting mixture was stirred for overnight at rt. The reaction mixture was quenched by water and extracted with EA (3*20 mL). The combined organic extracts were washed with brine (20 mL), dried over anhydrous Na₂SO₄, and concentrated under vacuum to yield a crude product which was directly purified by flash chromatography (0-100% ethyl acetate/petroleum ether) to afford 100 mg crude product as a white solid. The compound was separated by preparative HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 26% B to 45% B in 10 min, 45% B; Wave Length: 254/220 nm; RT1(min): 8.9; Number Of Runs: 0) to afford 3-tert-butyl-1-(2-oxo-1-{[3-(trifluoromethyl)phenyl]methyl}-3,4-dihydroquinolin-6-yl)urea (125.4 mg, 31.80%) as a white solid. MS (ESI): mass calcd. for C₂₂H₂₄F₃N₃O₂, 419.18 m/z, found 419.95 [M+H]⁺. ¹H NMR (300 MHz, DMSO-d6) δ 8.13 (s, 1H), 7.45-7.63 (m, 4H), 7.34 (d, J=2.4 Hz, 1H), 7.00 (dd, J=8.7, 2.5 Hz, 1H), 6.80 (d, J=8.8 Hz, 1H), 5.93 (s, 1H), 5.20 (s, 2H), 2.89 (dd, J=8.8, 5.7 Hz, 2H), 2.67 (dd, J=8.6, 5.8 Hz, 2H), 1.27 (s, 9H).

### Example 24: 3-tert-butyl-1-{1-[(2,5-dichlorophenyl)methyl]-2-oxo-3,4-dihydroquinolin-6-yl}urea

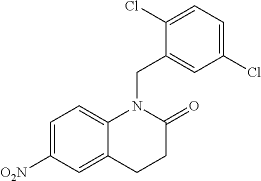 [SMILES: O=C1CCc2cc([N+](=O)[O-])ccc2N1Cc1cc(Cl)ccc1Cl]

### 1-[(2,5-dichlorophenyl)methyl]-6-nitro-3,4-dihydroquinolin-2-one

To a stirred solution of 6-nitro-3,4-dihydro-1H-quinolin-2-one (500 mg, 2.602 mmol, 1 equiv) in DMF (10 mL) was added 2-(bromomethyl)-1,4-dichlorobenzene (936.34 mg, 3.903 mmol, 1.5 equiv) and potassium carbonate (724.41 mg, 5.204 mmol, 2 equiv) at room temperature under nitrogen atmosphere. The final reaction mixture was stirred for 3 h at room temperature. The reaction was monitored by LCMS. The resulting mixture was quenched with water (20 mL) and extracted with EA (3×30 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EA (1:1) to afford 1-[(2,5-dichlorophenyl)methyl]-6-nitro-3,4-dihydroquinolin-2-one (500 mg, 54.72%) as a white solid. LC/MS: mass calcd. for C₁₆H₁₂C₁₂N₂O₃: 350.02 m/z, found:350.90 [M+H]⁺

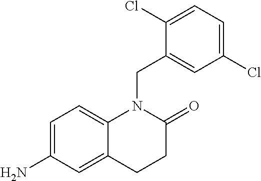 [SMILES: Nc1ccc2c(c1)CCC(=O)N2Cc1cc(Cl)ccc1Cl]

### 6-amino-1-[(2,5-dichlorophenyl)methyl]-3,4-dihydroquinolin-2-one

To a stirred solution of 1-[(2,5-dichlorophenyl)methyl]-6-nitro-3,4-dihydroquinolin-2-one (500 mg, 1.424 mmol, 1 equiv) in EtOH/H₂O (10 mL, 10/1) was added Fe (795.11 mg, 14.240 mmol, 10 equiv) and NH₄Cl (761.58 mg, 14.240 mmol, 10 equiv) at room temperature under nitrogen atmosphere. The final reaction mixture was stirred for 2 h at 60° C. The reaction was quenched with water at rt. After filtration, the filter cake was washed with EA (3×20 mL). The filtrate was added water (20 mL) and extracted with EA (3×20 mL). The combined organic layers were washed with brine (30 mL), dried over anhydrous Na₂SO₄. After filtration, the filtrate was concentrated under reduced pressure. The crude product was purified by silica gel column chromatography, eluted with PE/EA (1:1) to afford 6-amino-1-[(2,5-dichlorophenyl)methyl]-3,4-dihydroquinolin-2-one (280 mg, 61.23%) as a white solid. LC/MS: mass calcd. for C₁₆H₁₄C₁₂N₂O: 320.05 m/z, found:320.95 [M+H]⁺.

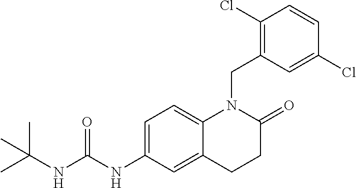 [SMILES: CC(C)(C)NC(=O)Nc1ccc2c(c1)CCC(=O)N2Cc1cc(Cl)ccc1Cl]

### 3-tert-butyl-1-{1-[(2,5-dichlorophenyl)methyl]-2-oxo-3,4-dihydroquinolin-6-yl}urea

To a stirred solution of 6-amino-1-[(2,5-dichlorophenyl)methyl]-3,4-dihydroquinolin-2-one (140 mg, 0.436 mmol, 1 equiv) and 2-isocyanato-2-methylpropane (129.63 mg, 1.308 mmol, 3 equiv) in DCM (10 mL) was added TEA (132.32 mg, 1.308 mmol, 3 equiv) dropwise at 0° C. under nitrogen atmosphere. The resulting mixture was stirred for 1 day at room temperature under nitrogen atmosphere. The reaction was monitored by LCMS. Quenched with water (10 mL) and extracted with EA (3×10 mL). The combined extracts were washed with water, brine, dried over Na₂SO₄, filtered, and concentrated. The crude product was purified by Prep-HPLC to afford 3-tert-butyl-1-{1-[(2,5-dichlorophenyl)methyl]-2-oxo-3,4-dihydroquinolin-6-yl}urea (20.8 mg, 11.34%) as a white solid. LC/MS: mass calcd for C₂₁H₂₃C₁₂N₃O₂: 419.12 m/z, found:420.05 [M+H]⁺. ¹H NMR (400 MHz, DMSO-d6) δ 8.16 (s, 1H), 7.56 (d, J=8.4 Hz, 1H), 7.40-7.37 (m, 2H), 7.01-6.95 (m, 2H), 6.60 (d, J=8.4 Hz, 1H), 5.94 (s, 1H), 5.05 (s, 2H), 2.94-2.90 (m, 2H), 2.72-2.69 (m, 2H), 1.27 (s, 9H).

### Example 25: 1-(tert-butyl)-3-(1-(3,5-dichlorobenzyl)-2-oxo-1,2,3,4-tetrahydroquinolin-6-yl) urea

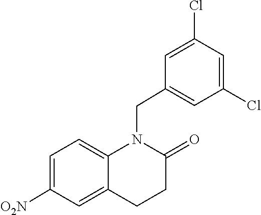 [SMILES: O=C1CCc2cc([N+](=O)[O-])ccc2N1Cc1cc(Cl)cc(Cl)c1]

### 1-(3,5-dichlorobenzyl)-6-nitro-3,4-dihydroquinolin-2(1H)-one

To a mixture of 6-nitro-3,4-dihydro-1H-quinolin-2-one (400 mg, 2.081 mmol, 1 equiv) and K₂CO₃ (869.29 mg, 6.243 mmol, 3 equiv) in DMF (20 mL) was added 1-(bromomethyl)-3,5-dichlorobenzene (749.07 mg, 3.122 mmol, 1.5 equiv). The reaction was stirred at rt for 2 hours. Quenched with water (30 mL) and extracted with EA (3×30 mL). The combined extracts were washed with water, brine, dried over Na₂SO₄, filtered, and concentrated. The residue was purified by column chromatography on silica gel with EA/PE (0-100%) to give 1-[(3,5-dichlorophenyl)methyl]-6-nitro-3,4-dihydroquinolin-2-one (647 mg, 88.51%) as a yellow solid. MS (ESI): mass calcd. for C₁₆H₁₂C₁₂N₂O₃: 351.18m/z, found 350.95 [M+H]⁺.

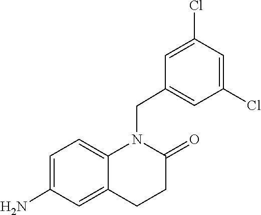 [SMILES: Nc1ccc2c(c1)CCC(=O)N2Cc1cc(Cl)cc(Cl)c1]

### 6-amino-1-(3,5-dichlorobenzyl)-3,4-dihydroquinolin-2(1H)-one

To a solution of 1-[(3,5-dichlorophenyl)methyl]-6-nitro-3,4-dihydroquinolin-2-one (500 mg, 1.424 mmol, 1 equiv) in MeOH/H₂O (11 mL, 10/1) was added Fe (397.55 mg, 7.120 mmol, 5 equiv) and NH₄Cl (380.79 mg, 7.120 mmol, 5 equiv). The reaction was stirred at 80° C. for 1 hours. After filtration, the filter cake was washed with EA (3×10 mL). The filtrate was added water (20 mL) and extracted with EA (3×20 mL). The combined extracts were washed with water, brine, dried over Na₂SO₄, filtered, and concentrated. The resulting mixture was concentrated under vacuum. This resulted in 6-amino-1-[(3,5-dichlorophenyl)methyl]-3,4-dihydroquinolin-2-one (418 mg, 91.40%) as a yellow solid. MS (ESI): mass calcd. for C₁₆H₁₄C₁₂N₂O: 321.20 m/z, found 320.95 [M+H]⁺.

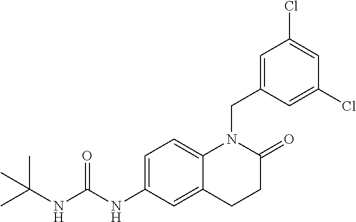 [SMILES: CC(C)(C)NC(=O)Nc1ccc2c(c1)CCC(=O)N2Cc1cc(Cl)cc(Cl)c1]

### 1-(tert-butyl)-3-(1-(3,5-dichlorobenzyl)-2-oxo-1,2,3,4-tetrahydroquinolin-6-yl)urea

To a solution of 6-amino-1-[(3,5-dichlorophenyl)methyl]-3,4-dihydroquinolin-2-one (100 mg, 0.311 mmol, 1 equiv) in DCM (10 mL) was added 2-isocyanato-2-methylpropane (92.59 mg, 0.933 mmol, 3 equiv) and TEA (94.51 mg, 0.933 mmol, 3 equiv) at 0° C. The reaction was stirred at rt for 2 days. Quenched with water (20 mL) and extracted with EA (3×20 mL). The combined extracts were washed with water, brine, dried over Na₂SO₄, filtered, and concentrated. The residue was purified by preparative HPLC using a XBridge Shield RP18 OBD Column, 30*150 mm, 5 μm column (eluent: 45% to 67% (v/v) CH₃CN and H₂O with 10 mmol/L NH₄HCO₃) to afford the title compound 3-tert-butyl-1-{1-[(3,5-dichlorophenyl)methyl]-2-oxo-3,4-dihydroquinolin-6-yl}urea (20 mg, 15.28%) as a white solid. MS (ESI): mass calcd. for C₂₁H₂₃C₁₂N₃O₂:420.33 m/z, found:420.15 [M+H]⁺. ¹H NMR (400 MHz, DMSO-d6) δ 8.04-8.23 (m, 1H), 7.39-7.49 (m, 1H), 7.22-7.39 (m, 3H), 6.94-7.11 (m, 1H), 6.65-6.88 (m, 1H), 5.80-6.01 (m, 1H), 5.00-5.21 (m, 2H), 2.81-2.98 (m, 2H), 2.60-2.78 (m, 2H), 1.25-1.33 (m, 9H).

### Example 26: 1-(tert-butyl)-3-(1-(3-fluorobenzyl)-2-oxo-1,2,3,4-tetrahydroquinolin-6-yl)urea

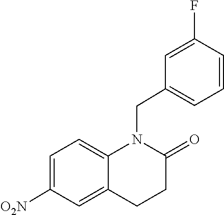 [SMILES: O=C1CCc2cc([N+](=O)[O-])ccc2N1Cc1cccc(F)c1]

### 1-(3-fluorobenzyl)-6-nitro-3,4-dihydroquinolin-2(1H)-one

To a solution of 6-nitro-3,4-dihydro-1H-quinolin-2-one (500 mg, 2.602 mmol, 1 equiv) in DMF (20 mL) was added K₂CO₃ (1086.62 mg, 7.806 mmol, 3 equiv) and (bromomethyl)-3-fluorobenzene (737.72 mg, 3.903 mmol, 1.5 equiv) at rt. The final reaction mixture was stirred for 1 h at rt. Quenched with water (20 mL) and extracted with EA (3×20 mL). The combined extracts were washed with water, brine, dried over Na₂SO₄, filtered, and concentrated. The residue was purified by column chromatography on silica gel with EA/PE (0-100%) to give 1-[(3-fluorophenyl)methyl]-6-nitro-3,4-dihydroquinolin-2-one (163 mg, 20.86%) as a yellow solid. MS (ESI): mass calcd. for C₁₆H₁₃FN₂O₃: 300.29 m/z, found 301.00 [M+H]⁺.

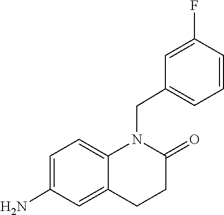 [SMILES: Nc1ccc2c(c1)CCC(=O)N2Cc1cccc(F)c1]

### 6-amino-1-(3-fluorobenzyl)-3,4-dihydroquinolin-2(1H)-one

To a solution of 1-[(3-fluorophenyl)methyl]-6-nitro-3,4-dihydroquinolin-2-one (155 mg, 0.516 mmol, 1 equiv) in MeOH/H₂O (22 mL, 10/1) was added Fe (144.13 mg, 2.580 mmol, 5 equiv) and NH₄Cl (138.05 mg, 2.580 mmol, 5 equiv). The reaction was stirred at 80° C. for 1 hour. The resulting mixture was filtered, the filter cake was washed with EA (3×20 mL). The filtrate was concentrated under reduced pressure. The residue was added water (20 mL) and extracted with EA (3×20 mL). The combined extracts were washed with water, brine, dried over Na₂SO₄, filtered, and concentrated. The residue was purified by column chromatography on silica gel with EA/PE (0-100%) to give 6-amino-1-[(3-fluorophenyl)methyl]-3,4-dihydroquinolin-2-one hydrochloride (100 mg, 63.15%) as a yellow oil. MS (ESI): mass calcd. for C₁₆H₁₅FN₂O: 270.31m/z, found 271.15 [M+H]⁺.

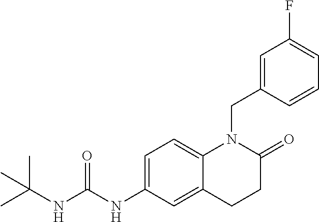 [SMILES: CC(C)(C)NC(=O)Nc1ccc2c(c1)CCC(=O)N2Cc1cccc(F)c1]

### 1-(tert-butyl)-3-(1-(3-fluorobenzyl)-2-oxo-1,2,3,4-tetrahydroquinolin-6-yl)urea

To a solution of 6-amino-1-[(3-fluorophenyl)methyl]-3,4-dihydroquinolin-2-one (90 mg, 0.333 mmol, 1 equiv) in DCM (10 mL) was added 2-isocyanato-2-methylpropane (99.02 mg, 0.999 mmol, 3 equiv) and Pyridine (79.01 mg, 0.999 mmol, 3 equiv). The reaction was stirred at rt for 3 days. Quenched with water (20 mL) and extracted with DCM (3×20 mL). The combined extracts were washed with water, brine, dried over Na₂SO₄, filtered, and concentrated. The residue was purified by preparative HPLC using a XBridge Prep OBD C18 Column, 30*150 mm, 5 μm column (eluent: 17% to 42% (v/v) CH₃CN and H₂O with 10 mmol/L NH₄HCO₃) to afford the title compound 3-tert-butyl-1-{1-[(3-fluorophenyl)methyl]-2-oxo-3,4-dihydroquinolin-6-yl}urea (20.7 mg, 16.83%) as a white solid. MS (ESI): mass calcd. for C₂₁H₂₄FN₃O₂:369.44 m/z, found:370.15 [M+H]⁺. ¹H NMR (400 MHz, DMSO-d6) δ 7.96-8.19 (m, 1H), 7.30-7.40 (m, 2H), 6.93-7.19 (m, 4H), 6.69-6.81 (m, 1H), 5.80-6.00 (m, 1H), 4.98-5.29 (m, 2H), 2.81-2.94 (m, 2H), 2.60-2.71 (m, 2H), 1.19-1.29 (m, 9H). ¹⁹F NMR (376 MHz, DMSO) δ (ppm): −113.2050.

### Example 27: 1-(tert-butyl)-3-(1-(4-fluorobenzyl)-2-oxo-1,2,3,4-tetrahydroquinolin-6-yl)urea

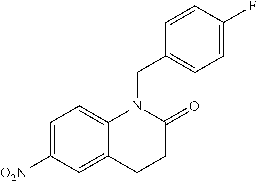 [SMILES: O=C1CCc2cc([N+](=O)[O-])ccc2N1Cc1ccc(F)cc1]

### 1-(4-fluorobenzyl)-6-nitro-3,4-dihydroquinolin-2(1H)-one

To a solution of 6-nitro-3,4-dihydro-1H-quinolin-2-one (500 mg, 2.602 mmol, 1 equiv) and K₂CO₃ (1086.62 mg, 7.806 mmol, 3 equiv) in DMF (10 mL) was added 1-(bromomethyl)-4-fluorobenzene (737.78 mg, 3.903 mmol, 1.5 equiv). The reaction was stirred at rt for 2 hours. Quenched with water (20 mL) and extracted with EA (3×20 mL). The combined extracts were washed with water, brine, dried over Na₂SO₄, filtered, and concentrated. The residue was purified by column chromatography on silica gel with EA/PE (0-100%) to give 1-[(4-fluorophenyl)methyl]-6-nitro-3,4-dihydroquinolin-2-one (630 mg, 80.64%) as a yellow solid. MS(ESI): mass calcd. for C₁₆H₁₃FN₂O₃: 300.29 m/z, found 301.00 [M+H]⁺.

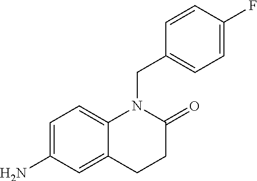 [SMILES: Nc1ccc2c(c1)CCC(=O)N2Cc1ccc(F)cc1]

### 6-amino-1-(4-fluorobenzyl)-3,4-dihydroquinolin-2(1H)-one

To a solution of 1-[(4-fluorophenyl)methyl]-6-nitro-3,4-dihydroquinolin-2-one (400 mg, 1.332 mmol, 1 equiv) in MeOH/H₂O (22 mL, 10/1) was added Fe (371.94 mg, 6.660 mmol, 5 equiv) and NH₄Cl (356.26 mg, 6.660 mmol, 5 equiv). The reaction was stirred for 1 hour at 80° C. The reaction was quenched with water at rt. After filtration, the filter cake was washed with EA (3×20 mL). The filtrate was added water (20 mL) and extracted with EA (3×20 mL). The combined extracts were washed with water, brine, dried over Na₂SO₄, filtered, and concentrated. The resulting mixture was concentrated under vacuum. This resulted in 6-amino-1-[(4-fluorophenyl)methyl]-3,4-dihydroquinolin-2-one (325 mg, 90.26%) as a yellow solid. MS (ESI): mass calcd. for C₁₆H₁₅FN₂O: 270.31m/z, found 271.05 [M+H]⁺.

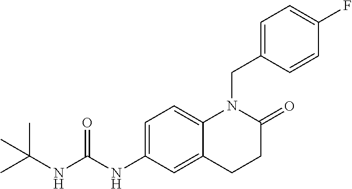 [SMILES: CC(C)(C)NC(=O)Nc1ccc2c(c1)CCC(=O)N2Cc1ccc(F)cc1]

### 1-(tert-butyl)-3-(1-(4-fluorobenzyl)-2-oxo-1,2,3,4-tetrahydroquinolin-6-yl)urea

To a solution of 6-amino-1-[(4-fluorophenyl)methyl]-3,4-dihydroquinolin-2-one (150 mg, 0.555 mmol, 1 equiv) in DCM (20 mL) was added 2-isocyanato-2-methylpropane (165.03 mg, 1.665 mmol, 3 equiv) and Pyridine (131.68 mg, 1.665 mmol, 3 equiv). The reaction was stirred at rt for 3 days. Quenched with water (10 mL) and extracted with EA (3×10 mL). The combined extracts were washed with water, brine, dried over Na₂SO₄, filtered, and concentrated. The residue was purified by preparative HPLC using a XBridge Prep OBD C18 Column, 30*150 mm, 5 μm column (eluent: 17% to 42% (v/v) CH₃CN and H₂O with 10 mmol/L NH₄HCO₃) to afford the title compound 3-tert-butyl-1-{1-[(4-fluorophenyl)methyl]-2-oxo-3,4-dihydroquinolin-6-yl}urea (5.3 mg, 2.59%) as a white solid. MS (ESI): mass calcd. for C₂₁H₂₄FN₃O₂:369.44 m/z, found:370.15 [M+H]⁺. ¹H NMR (400 MHz, DMSO-d₆) δ 7.11-7.40 (m, 3H), 6.96-7.11 (m, 3H), 6.81-6.91 (m, 1H), 5.10-5.22 (m, 2H), 2.82-3.03 (m, 2H), 2.59-2.82 (m, 2H), 1.29-1.41 (m, 9H). ¹⁹F NMR (376 MHz, DMSO) δ (ppm): −117.9957.

### Example 28: 1-(tert-butyl)-3-(2-oxo-1-(1-phenylpropyl)-1,2,3,4-tetrahydroquinolin-6-yl)urea

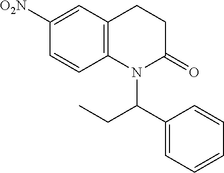 [SMILES: CCC(c1ccccc1)N1C(=O)CCc2cc([N+](=O)[O-])ccc21]

### 6-nitro-1-(1-phenylpropyl)-3,4-dihydroquinolin-2-one

To a solution of 6-nitro-3,4-dihydro-1H-quinolin-2-one (400 mg, 2.081 mmol, 1 equiv) in DMF (8 mL) was added K₂CO₃ (863.0 mg, 6.244 mmol, 3 equiv) and (1-bromopropyl)benzene (455.8 mg, 2.289 mmol, 1.1 equiv). The resulting mixture was stirred for 16 h at rt. The mixture was diluted with EtOAc (100 mL), washed with water (3×30 mL), brine (2×30 mL) and concentrated under vacuum to yield a crude product which was directly purified by silica gel column, eluted with PE/EA (3:1) to afford 6-nitro-1-(1-phenylpropyl)-3,4-dihydroquinolin-2-one (150 mg, 22.24%) as a yellow solid. MS (ESI): mass calcd. for C₁₈H₁₈N₂O₃ 310.13 m/z, found 311.25 [M+H]⁺.

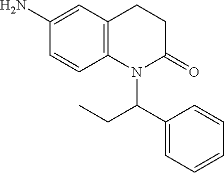 [SMILES: CCC(c1ccccc1)N1C(=O)CCc2cc(N)ccc21]

### 6-amino-1-(1-phenylpropyl)-3,4-dihydroquinolin-2-one

To a solution of 6-nitro-1-(1-phenylpropyl)-3,4-dihydroquinolin-2-one (130 mg, 0.419 mmol, 1 equiv) in EtOH (8 mL) and H₂O (2 mL) was added Fe (233.9 mg, 4.189 mmol, 10 equiv) and NH₄Cl (224.0 mg, 4.189 mmol, 10 equiv). The resulting mixture was stirred for 2 h at 60° C. The mixture was diluted with EtOAc (100 mL), washed with water (3×30 mL), brine (2×30 mL) and concentrated under vacuum to yield a crude product which was directly purified by silica gel column, eluted with PE/EA (2:1) to afford 6-amino-1-(1-phenylpropyl)-3,4-dihydroquinolin-2-one (100 mg, 85.15%) as a yellow solid. MS (ESI): mass calcd. for C₁₈H₂₀N₂O 280.16 m/z, found 281.10 [M+H]⁺.

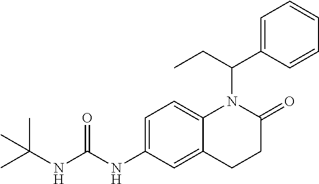 [SMILES: CCC(c1ccccc1)N1C(=O)CCc2cc(NC(=O)NC(C)(C)C)ccc21]

### 1-(tert-butyl)-3-(2-oxo-1-(1-phenylpropyl)-1,2,3,4-tetrahydroquinolin-6-yl)urea

To a solution of 6-amino-1-(1-phenylpropyl)-3,4-dihydroquinolin-2-one (100 mg, 1.502 mmol, 1 equiv) in DCM (8 mL) was added 2-isocyanato-2-methylpropane (106.0 mg, 1.070 mmol, 3 equiv) and TEA (108.2 mg, 1.070 mmol, 3 equiv). The resulting mixture was stirred for overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: Aeris PEPTIDE 5 um XB-C18 Axia, 21.2 mm×250 mm, 5 μm; Mobile Phase A: Water(10 mmol/L NH₄HCO₃), Mobile Phase B: ACN; Flow rate: 60 mL/min; Gradient: 39% B to 61% B in 10.5 min; Wave Length: 220 nm; RT1(min): 7.9; Number Of Runs: 2) to afford 1-(tert-butyl)-3-(2-oxo-1-(1-phenylpropyl)-1,2,3,4-tetrahydroquinolin-6-yl)urea (29.8 mg, 21.98%) as a white solid. MS (ESI): mass calcd. for C₂₃H₂₉N₃O₂, 379.23 m/z, found 380.20 [M+H]⁺. ¹H NMR (300 MHz, DMSO-d6) δ 8.09 (s, 1H), 7.20-7.41 (m, 6H), 6.81 (dd, J=8.8, 2.5 Hz, 1H), 6.49 (d, J=8.8 Hz, 1H), 5.95 (d, J=13.1 Hz, 2H), 2.74-2.92 (m, 2H), 2.56-2.74 (m, 2H), 2.31-2.40 (m, 1H), 2.04-2.22 (m, 1H), 1.25 (s, 9H), 0.77 (t, J=7.2 Hz, 3H).

### Example 29: 1-(1-benzyl-3,3-dimethyl-2-oxo-1,2,3,4-tetrahydroquinolin-6-yl)-3-(tert-butyl)urea

### Synthetic Scheme

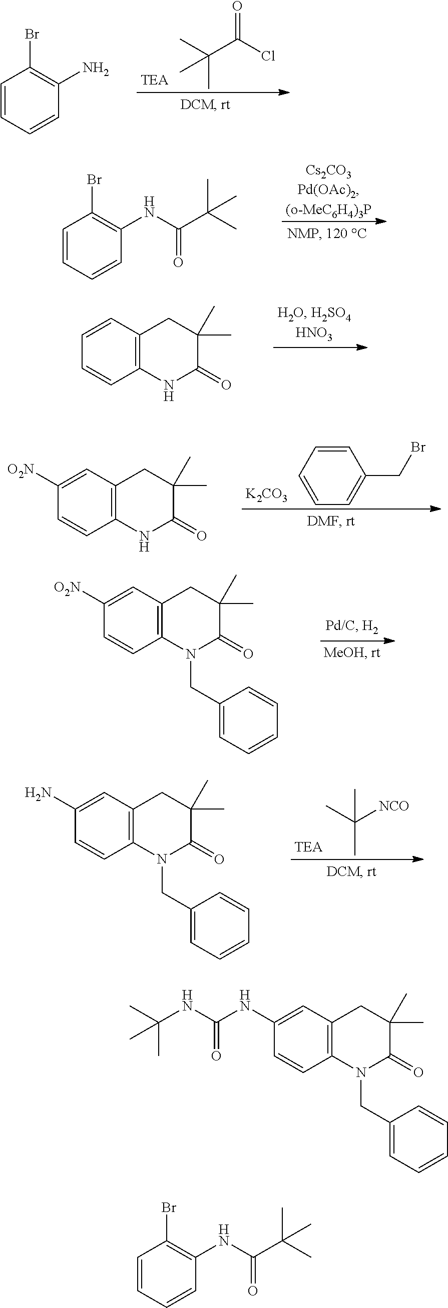

### N-(2-bromophenyl)pivalamide

To a stirred solution of 0-bromoaniline (3 g, 17.439 mmol, 1 equiv) in anhydrous DCM (50 mL) was added triethylamine (2.29 g, 22.671 mmol, 1.3 equiv) followed by 2,2-dimethylpropanoyl chloride (2.10 g, 17.439 mmol, 1 equiv) at 0° C. The reaction mixture was stirred for 16 h at rt. After completion of reaction, the reaction mixture was quenched by addition of water (50 mL). The aqueous layer was extracted with DCM (50 mL). The combined organic phase was washed with brine (30 mL), dried over anhydrous sodium sulfate and concentrated under reduced pressure to give crude product which was further purified by silica gel column, eluted with PE/EA (3:1) to afford desired compound N-(2-bromophenyl)-2,2-dimethylpropanamide (2.7 g, 60.44%) as a yellow solid. MS (ESI): mass calcd. for C₁₁H₁₄BrNO: 255.03 m/z, found 256.05 [M+H]⁺.

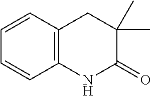

### 3,3-dimethyl-3,4-dihydroquinolin-2(1H)-one

To a stirred solution of N-(2-bromophenyl)-2,2-dimethylpropanamide (400 mg, 1.562 mmol, 1 equiv) in NMP (20 mL, 207.398 mmol, 132.81 equiv) was added Cs₂CO₃ (1.02 g, 3.124 mmol, 2 equiv), tris(4-methylphenyl)phosphane (95.06 mg, 0.312 mmol, 0.2 equiv), Pd(OAc)₂ (35.06 mg, 0.156 mmol, 0.1 equiv) and TBHP (42.22 mg, 0.469 mmol, 0.3 equiv) at rt and stirred for overnight at 140° C. under N₂. The reaction mixture was concentrated under reduced pressure to give crude product which was further purified by silica, gel column PE/EA (3:1) to afford desired compound 3,3-dimethyl-1,4-dihydroquinolin-2-one (0.2 g, 73.26%). MS (ESI): mass calcd. for C₁₁H₁₃NO: 175.10 m/z, found 176.25 [M+H]⁺.

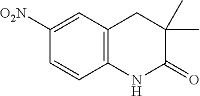 [SMILES: CC1(C)Cc2cc([N+](=O)[O-])ccc2NC1=O]

### 3,3-dimethyl-6-nitro-3,4-dihydroquinolin-2(1H)-one

To a solution of 3,3-dimethyl-3,4-dihydroquinolin-2(1H)-one (0.2 g, 1.14 mmol, 1 equiv) in H₂O (2 mL) and H₂SO₄ (9 mL) was added HNO₃ (0.2 mL) at −10° C. The reaction was stirred for 2 h at −10-0° C. The reaction mixture was quenched by water. The mixture was acidified pH=7 with NaHCO₃ (aq.) and then extracted with EA (3×50 mL). The combined organic extracts were washed with brine (50 mL), dried over anhydrous Na₂SO₄, and concentrated under vacuum to yield a crude product which was directly purified by silica gel column, eluted with PE/EA (3:1) to afford 3,3-dimethyl-6-nitro-3,4-dihydroquinolin-2(1H)-one (0.15 g, 60.0%) as a white solid. MS (ESI): mass calcd. for C₁₁H₁₂N₂O₃, 220.08 m/z, found 221.05 [M+H]⁺.

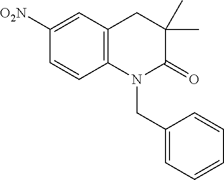 [SMILES: CC1(C)Cc2cc([N+](=O)[O-])ccc2N(Cc2ccccc2)C1=O]

### 1-benzyl-3,3-dimethyl-6-nitro-3,4-dihydroquinolin-2(1H)-one

To a stirred solution of 3,3-dimethyl-6-nitro-1,4-dihydroquinolin-2-one (150 mg, 0.681 mmol, 1 equiv) in DMF (3 mL) was added K₂CO₃ (322.39 mg, 2.316 mmol, 3 equiv) and benzyl bromide (158.43 mg, 0.926 mmol, 1.2 equiv) at rt and stirred for overnight. The resulting mixture was extracted with EA (3×30 mL), dried over anhydrous Na₂SO₄. After filtration, the filtrate was concentrated under reduced pressure to yield a crude product which was directly purified by silica gel column, eluted with PE/EA (3:1) to afford 1-benzyl-3,3-dimethyl-6-nitro-3,4-dihydroquinolin-2(1H)-one (150 mg, 71.09%) as a white solid. MS (ESI): mass calcd. for C₁₈H₁₈N₂O₃: 310.13 m/z, found 311.10 [M+H]⁺.

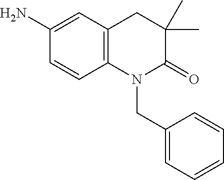 [SMILES: CC1(C)Cc2cc(N)ccc2N(Cc2ccccc2)C1=O]

### 6-amino-1-benzyl-3,3-dimethyl-3,4-dihydroquinolin-2(1H)-one

To a solution of 1-benzyl-3,3-dimethyl-6-nitro-3,4-dihydroquinolin-2(1H)-one (130 mg, 0.419 mmol, 1 equiv) in 10 mL MeOH was added Pd/C (30 mg) under nitrogen atmosphere. The mixture was hydrogenated at room temperature for 50 min under hydrogen atmosphere using a hydrogen balloon, then filtered through a Celite pad and concentrated under reduced pressure to afford 6-amino-1-benzyl-3,3-dimethyl-4H-quinolin-2-one (100 mg, 85.15%) as a white solid. MS (ESI): mass calcd. for C₁₈H₂₀N₂O: 280.16 m/z, found 281.25 [M+H]⁺.

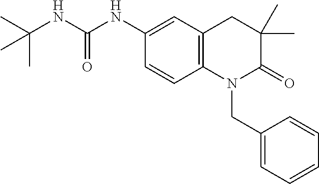 [SMILES: CC(C)(C)NC(=O)Nc1ccc2c(c1)CC(C)(C)C(=O)N2Cc1ccccc1]

### 1-(1-benzyl-3,3-dimethyl-2-oxo-1,2,3,4-tetrahydroquinolin-6-yl)-3-(tert-butyl)urea

To a stirred solution of 6-amino-1-benzyl-3,3-dimethyl-4H-quinolin-2-one (50 mg, 0.178 mmol, 1 equiv) in anhydrous DCM (5 mL) was added TEA (54.14 mg, 0.534 mmol, 3 equiv) 2-isocyanato-2-methylpropane (70.72 mg, 0.712 mmol, 4 equiv) at 0° C., and stirred for overnight at rt. After completion of reaction, the reaction mixture was concentrated under reduced pressure to give crude product which was further purified by HPLC with the following conditions Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 44% B to 60% B in 8.5 min; Wave Length: 220 nm; RT1(min): 6.9; Number Of Runs: 3; to afford 1-(1-benzyl-3,3-dimethyl-2-oxo-1,2,3,4-tetrahydroquinolin-6-yl)-3-(tert-butyl)urea (5.6 mg, 8.26%) as a white solid. MS (ESI):mass calcd. for C₂₃H₂₉N₃O₂: 379.23 m/z, found 380.20 [M+H]⁺. ¹H NMR (300 MHz, Methanol-d4) δ 7.18-7.34 (m, 6H), 7.01-7.04 (m, 1H), 6.82 (d, J=8.7 Hz, 1H), 5.16 (s, 2H), 2.84 (s, 2H), 1.36 (s, 9H), 1.23 (s, 6H).

### Example 30: 1-(tert-butyl)-3-(1-(2-hydroxy-1-phenylethyl)-2-oxo-1,2,3,4-tetrahydroquinolin-6-yl)urea

### Synthetic Scheme

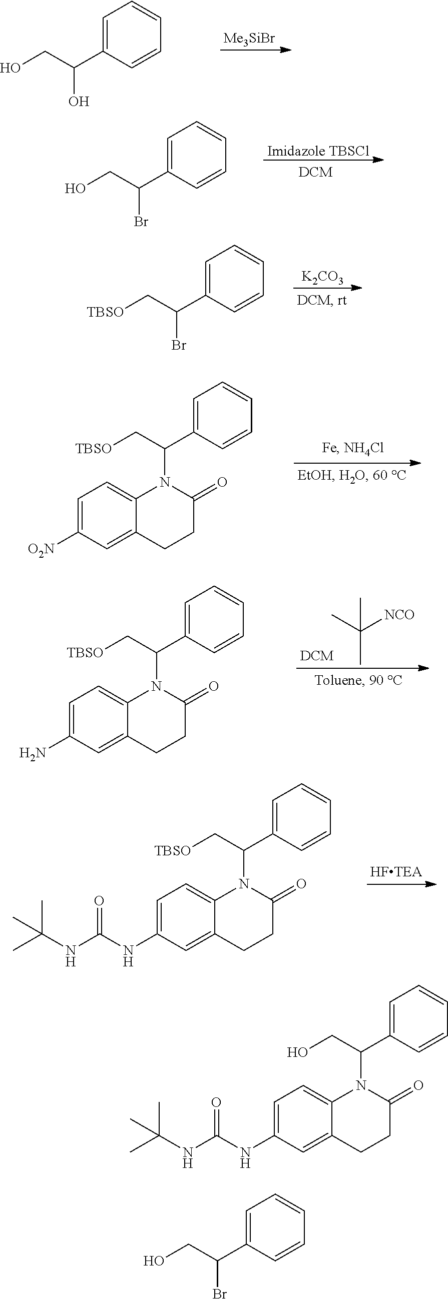

### 2-bromo-2-phenylethan-1-ol

Into a 40 mL round-bottom flask was added 2-bromo-2-phenylethan-1-ol (3 g, 21.713 mmol, 1 equiv) and bromotrimethylsilane (3.66 g, 23.884 mmol, 1.1 equiv) at rt. The resulting mixture was stirred for additional overnight at rt. The resulting mixture was concentrated under reduced pressure. The residue was purified by silica gel column chromatography, eluted with PE/EA (5:1) afford 2-bromo-2-phenylethanol (2.7 g, 61.85%) as a yellow semi-solid.

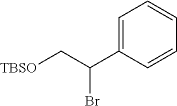 [SMILES: CC(C)(C)[Si](C)(C)OCC(Br)c1ccccc1]

### (2-bromo-2-phenylethoxy)(tert-butyl)dimethylsilane

To a stirred solution of 2-bromo-2-phenylethanol (1.4 g, 6.963 mmol, 1 equiv) in anhydrous DCM (30 mL) was added t-butyldimethylchlorosilane (2.10 g, 13.926 mmol, 2 equiv) and Imidazole (1.42 g, 20.889 mmol, 3 equiv) at rt and stirred for 1 h. The reaction progress was monitored by TLC. The residue was purified by silica gel column chromatography, eluted with EA (35%-58%) to afford (2-bromo-2-phenylethoxy)(tert-butyl)dimethylsilane (1.2 g, 54.65%) as a yellow semi-solid.

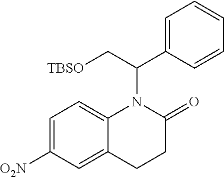 [SMILES: CC(C)(C)[Si](C)(C)OCC(c1ccccc1)N1C(=O)CCc2cc([N+](=O)[O-])ccc21]

### 1-(2-((tert-butyldimethylsilyl)oxy)-1-phenylethyl)-6-nitro-3,4-dihydroquinolin-2(1H)-one

To a stirred solution of (2-bromo-2-phenylethoxy)(tert-butyl)dimethylsilane (500 mg, 1.586 mmol, 1 equiv) in dimethylformamide (10 mL) was added 6-nitro-3,4-dihydro-1H-quinolin-2-one (274.25 mg, 1.427 mmol, 0.9 equiv) and Cs₂CO₃ (1549.92 mg, 4.758 mmol, 3 equiv) at rt and stirred for overnight. The reaction progress was monitored by LCMS. The resulting mixture was extracted with EA (3×30 mL), dried over anhydrous Na₂SO₄. After filtration, the filtrate was concentrated under reduced pressure. The residue was purified by silica gel column chromatography, eluted with PE/EA (3:1) to afford 1-(2-((tert-butyldimethylsilyl)oxy)-1-phenylethyl)-6-nitro-3,4-dihydroquinolin-2(1H)-one (350 mg, 51.74%) as a yellow solid. MS (ESI): mass calcd. for C₂₃H₃₀N₂O₄Si: 426.20 m/z, found 427.15 [M+H]⁺.

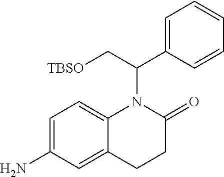 [SMILES: CC(C)(C)[Si](C)(C)OCC(c1ccccc1)N1C(=O)CCc2cc(N)ccc21]

### 6-amino-1-(2-((tert-butyldimethylsilyl)oxy)-1-phenylethyl)-3,4-dihydroquinolin-2(1H)-one

To a solution of 1-(2-((tert-butyldimethylsilyl)oxy)-1-phenylethyl)-6-nitro-3,4-dihydroquinolin-2(1H)-one (80 mg, 0.188 mmol, 1 equiv) in 10 mL MeOH was added Pd/C (10%, 50 mg) under nitrogen atmosphere in a 50 mL. The mixture was hydrogenated at room temperature for 1 h under hydrogen atmosphere using a hydrogen balloon, filtered through a Celite pad and concentrated under reduced pressure to afford 6-amino-1-(2-((tert-butyldimethylsilyl)oxy)-1-phenylethyl)-3,4-dihydroquinolin-2(1H)-one (75 mg, 100.0%) as a white solid. MS (ESI): mass calcd. for C₂₃H₃₂N₂O₂S: 396.22 m/z, found 397.30 [M+H]⁺.

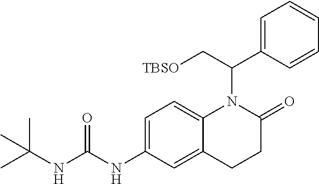 [SMILES: CC(C)(C)NC(=O)Nc1ccc2c(c1)CCC(=O)N2C(CO[Si](C)(C)C(C)(C)C)c1ccccc1]

### 1-(tert-butyl)-3-(1-(2-((tert-butyldimethylsilyl)oxy)-1-phenylethyl)-2-oxo-1,2,3,4-tetrahydroquinolin-6-yl)urea

To a stirred solution of 6-amino-1-(2-((tert-butyldimethylsilyl)oxy)-1-phenylethyl)-3,4-dihydroquinolin-2(1H)-one (100 mg, 0.252 mmol, 1 equiv) in DCM (10 mL) was added 2-isocyanato-2-methylpropane (74.99 mg, 0.756 mmol, 3 equiv) and toluene (5 mL, 46.993 mmol, 186.38 equiv) at rt and stirred for overnight at 90° C. The reaction progress was monitored by LCMS. After completion of reaction, the reaction mixture was concentrated under reduced pressure to give crude product. MS (ESI): mass calcd. for C₂₈H₄₁N₃O₃Si: 495.29 m/z, found 496.30 [M+H]⁺.

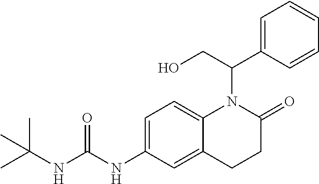 [SMILES: CC(C)(C)NC(=O)Nc1ccc2c(c1)CCC(=O)N2C(CO)c1ccccc1]

### 1-(tert-butyl)-3-(1-(2-hydroxy-1-phenylethyl)-2-oxo-1,2,3,4-tetrahydroquinolin-6-yl)urea

To a stirred solution of 1-(tert-butyl)-3-(1-(2-((tert-butyldimethylsilyl)oxy)-1-phenylethyl)-2-oxo-1,2,3,4-tetrahydroquinolin-6-yl)urea (crude) in tetrahydrofuran (5 mL) was added Triethylamine trihydrofluoride (300 mg) and stirred for 2 h at 60° C. The reaction progress was monitored by LCMS. After completion of reaction, the reaction mixture was concentrated under reduced pressure to give crude product which was further purified by HPLC with the following conditions Column: Xselect CSH F-Phenyl OBD column, 19*250 mm, 5 μm; Mobile Phase A: Water(10 mmol/L NH₄HCO₃), Mobile Phase B: ACN; Flow rate: 25 mL/min; Gradient: 45% B to 75% B in 7 min, 75% B; Wave Length: 254/220 nm; RT1(min): 6.4; Number Of Runs: 0 to afford 3-tert-butyl-1-[1-(2-hydroxy-1-phenylethyl)-2-oxo-3,4-dihydroquinolin-6-yl]urea (4 mg, 10.30%) as a white solid. MS (ESI):mass calcd. for C₂₂H₂₇N₃O₃: 381.21 m/z, found 382.20 [M+H]⁺. ¹H NMR (300 MHz, DMSO-d₆) δ 8.09 (s, 1H), 7.33 (d, J=4.0 Hz, 5H), 7.25 (q, J=4.2 Hz, 1H), 6.87 (dd, J=8.8, 2.5 Hz, 1H), 6.60 (d, J=8.8 Hz, 1H), 5.89 (d, J=13.5 Hz, 1H), 5.71 (d, J=7.1 Hz, 1H), 5.01 (t, J=5.5 Hz, 1H), 4.26 (dt, J=11.4, 6.0 Hz, 1H), 4.07 (dt, J 11.1, 6.1 Hz, 1H), 2.93-2.77 (m, 2H), 2.58 (q, J=6.5 Hz, 2H), 1.26 (s, 9H).

### Example 31: 1-(1-benzyl-2-oxo-3H-indol-5-yl)-3-tert-butylurea

### Synthetic Scheme

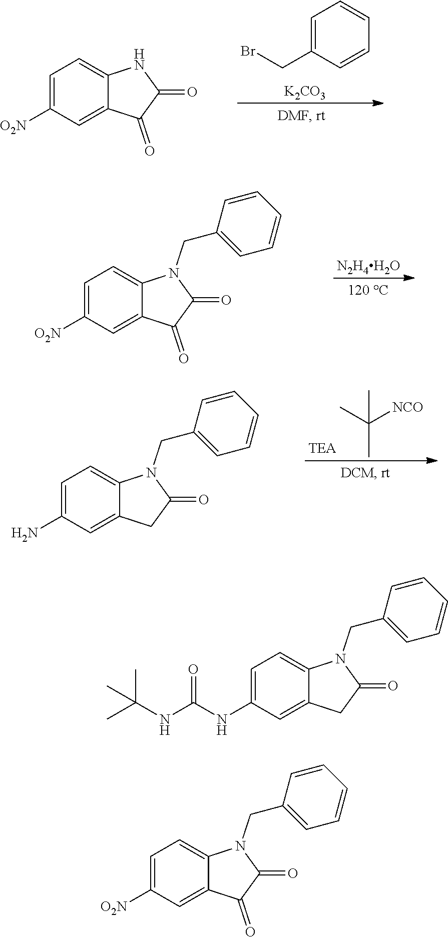

### 1-benzyl-5-nitroindole-2,3-dione

To a solution of 1H-indole-2,3-dione, 5-nitro (1 g, 5.205 mmol, 1 equiv) and benzyl bromide (979.24 mg, 5.726 mmol, 1.1 equiv) in dimethylformamide (10 mL) was added K₂CO₃ (1438.66 mg, 10.41 mmol, 2 equiv). The reaction was stirred at rt for 3h. Quenched with water (40 mL) and extracted with EA (3×40 mL). The combined extracts were washed with water, brine, dried over Na₂SO₄, filtered, and concentrated. The residue was purified by column chromatography on silica gel with EA/PE (0-100%) to give 1-benzyl-5-nitroindole-2,3-dione (665 mg, 45.27%) as a white solid. MS (ESI): mass calcd for C₁₅H₁₀N₂O₄: 282.06 m/z, found 283.15[M+H]⁺.

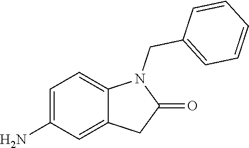 [SMILES: Nc1ccc2c(c1)CC(=O)N2Cc1ccccc1]

### benzyl-5-nitro-3H-indol-2-one

To a solution of 1-benzyl-5-nitroindole-2,3-dione (500 mg, 1.771 mmol, 1 equiv) in Hydrazinium hydroxide solution (5 mL) was stirred at 120° C. for 3h. Quenched with water (20 mL) and extracted with EA (3×20 mL). The combined extracts were washed with water, brine, dried over Na₂SO₄, filtered, and concentrated. The residue was purified by column chromatography on silica gel with EA/PE (0-100%) to 1-benzyl-5-nitro-3H-indol-2-one (437 mg, 91.96%) as a black solid. MS (ESI): mass calcd for C₁₅H₁₄N₂O: 238.11 m/z, found 239.15[M+H]⁺.

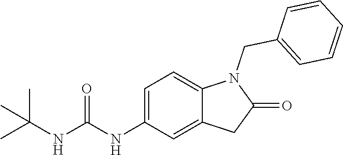 [SMILES: CC(C)(C)NC(=O)Nc1ccc2c(c1)CC(=O)N2Cc1ccccc1]

### 1-(1-benzyl-2-oxo-3H-indol-5-yl)-3-tert-butylurea

To a solution of 5-amino-1-benzyl-3H-indol-2-one (200 mg, 0.839 mmol, 1 equiv) and 2-isocyanato-2-methylpropane (124.2 mg, 1.253 mmol, 1.49 equiv) in DCM (2 mL) was added TEA (253.2 mg, 2.502 mmol, 2.98 equiv). The reaction was stirred at rt for 16h. Quenched with water (10 mL) and extracted with EA (3×10 mL). The combined extracts were washed with water, brine, dried over Na₂SO₄, filtered, and concentrated. The residue was purified by preparative HPLC using a XBridge Prep OBD C18 Column 150 mm×30 mm×5 μm column (eluent: 17% to 42% (v/v) CH₃CN and H₂O with 10 mmol/L NH₄HCO₃) to afford the title compound 1-(1-benzyl-2-oxo-3H-indol-5-yl)-3-tert-butylurea (26.3 mg, 9.27%) as a white solid. LC/MS (ESI): mass calcd. for C₂₀H₂₃N₃O₂:337.18 m/z, found:338.00 [M+H]⁺. ¹H NMR (400 MHz, DMSO-d6) δ 8.08 (s, 1H), 7.42 (m, 1H), 7.23-7.34 (m, 5H), 7.02 (m, 1H), 6.70 (m, 1H), 5.88 (s, 1H), 4.84 (s, 2H), 3.63 (s, 2H), 1.27 (s, 9H).

### Example 32: 1-(1-benzyl-3-isopropyl-2-oxo-1,2,3,4-tetrahydroquinolin-6-yl)-3-(tert-butyl)urea

### Synthetic Scheme

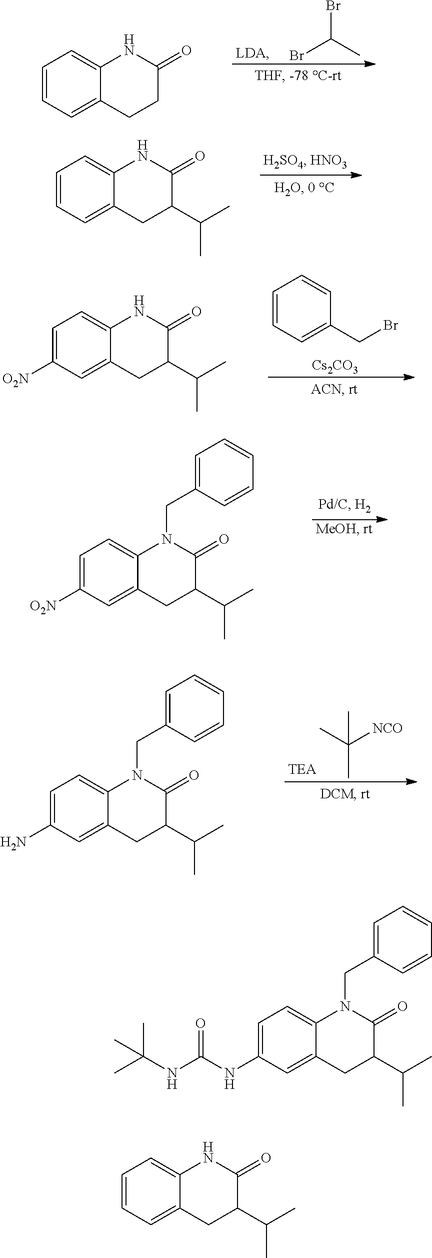

### 3-isopropyl-3,4-dihydroquinolin-2(1H)-one

To a solution of 3,4-dihydro-1H-quinolin-2-one (2 g, 13.589 mmol, 1 equiv) in THF (70 mL) was added 2M LDA (in THF) (17 mL) at −78° C. under N₂. The resulting mixture was stirred 30 min at −78° C. The reaction mixture was added 2-bromopropane (2.01 g, 16.343 mmol, 1.2 equiv) at −78° C. The resulting mixture was stirred 30 min at −78° C. and then stirred 16 h at rt. The reaction mixture was quenched by water (50 mL) and extracted with EA (3×70 mL). The combined organic extracts were washed with brine (50 mL), dried over anhydrous Na₂SO₄, and concentrated under vacuum to yield a crude product which was directly purified by silica gel column, eluted with PE/EA (3:1) to afford 3-isopropyl-3,4-dihydro-1H-quinolin-2-one (1 g, 21.98%) as a white solid. MS (ESI): mass calcd. for C₁₂H₁₅NO, 189.12 m/z, found 190.30 [M+H]⁺.

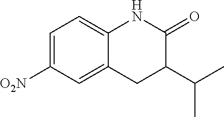 [SMILES: CC(C)C1Cc2cc([N+](=O)[O-])ccc2NC1=O]

### 3-isopropyl-7-nitro-3,4-dihydroquinolin-2(1H)-one

To a solution of 3-isopropyl-3,4-dihydro-1H-quinolin-2-one (510 mg, 2.695 mmol, 1 equiv) in H₂O (2 mL) and H₂SO₄ (9 mL) was added HNO₃ (0.2 mL) at −10° C. The resulting mixture was stirred at −10° C. until the starting material was totally consumed by LCMS. The reaction mixture was quenched by water. The mixture was acidified pH=7 with NaHCO₃ (aq.) and then extracted with EA (3×50 mL). The combined organic extracts were washed with brine (50 mL), dried over anhydrous Na₂SO₄, and concentrated under vacuum to yield a crude product which was directly purified by silica gel column, eluted with PE/EA (3:1) to afford 3-isopropyl-7-nitro-3,4-dihydro-1H-quinolin-2-one (200 mg, 24.28%) as a white solid. MS (ESI): mass calcd. for C₁₂H₁₄N₂O₃, 234.10 m/z, found 235.05 [M+H]⁺.

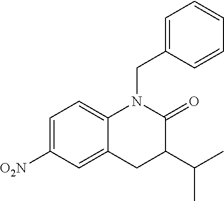 [SMILES: CC(C)C1Cc2cc([N+](=O)[O-])ccc2N(Cc2ccccc2)C1=O]

### 1-benzyl-3-isopropyl-7-nitro-3,4-dihydroquinolin-2(1H)-one

To a solution of 3-isopropyl-7-nitro-3,4-dihydro-1H-quinolin-2-one (140 mg, 0.598 mmol, 1 equiv) in DMF (5 mL) was added K₂CO₃ (247.7 mg, 1.793 mmol, 3 equiv) and benzyl bromide (112.4 mg, 0.657 mmol, 1.1 equiv). The resulting mixture was stirred 16 h at rt. The mixture was diluted with EtOAc (100 mL), washed with water (3×30 mL), brine (2×30 mL) and concentrated under vacuum to yield a crude product which was directly purified by silica gel column, eluted with PE/EA (3:1) to afford 1-benzyl-3-isopropyl-7-nitro-3,4-dihydroquinolin-2(1H)-one (100 mg, 49.05%) as a yellow solid. MS (ESI): mass calcd. for C₁₉H₂₀N₂O₃ 324.15 m/z, found 325.05 [M+H]⁺.

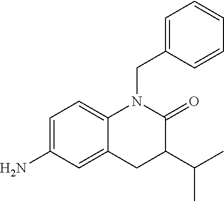 [SMILES: CC(C)C1Cc2cc(N)ccc2N(Cc2ccccc2)C1=O]

### 7-amino-1-benzyl-3-isopropyl-3,4-dihydroquinolin-2(1H)-one

To a solution of 1-benzyl-3-isopropyl-7-nitro-3,4-dihydroquinolin-2(1H)-one (140 mg, 0.432 mmol, 1 equiv) in MeOH (10 mL) was added Pd/C (30 mg). The reaction mixture was then stirred for 1 h under hydrogen atmosphere (balloon). The reaction mixture was filtered through a pad of Celite and concentrated under reduced pressure to afford 7-amino-1-benzyl-3-isopropyl-3,4-dihydroquinolin-2(1H)-one (100 mg, 77.79%) as a yellow solid. MS (ESI): mass calcd. for C₁₉H₂₂N₂O, 294.17 m/z, found 295.10 [M+H].

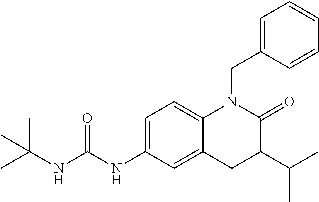 [SMILES: CC(C)C1Cc2cc(NC(=O)NC(C)(C)C)ccc2N(Cc2ccccc2)C1=O]

### 1-(1-benzyl-3-isopropyl-2-oxo-1,2,3,4-tetrahydroquinolin-6-yl)-3-(tert-butyl)urea

To a solution of 7-amino-1-benzyl-3-isopropyl-3,4-dihydroquinolin-2(1H)-one (100 mg, 0.340 mmol, 1 equiv) in DCM (5 mL) was added TEA (103.1 mg, 1.019 mmol, 3 equiv) and 2-isocyanato-2-methylpropane (101.0 mg, 1.019 mmol, 3 equiv). The resulting mixture was stirred for 16 h at rt. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XSelect CSH Fluoro Phenyl, 30*150 mm, 5 μm; Mobile Phase A: Water(10 mmol/L NH₄HCO₃), Mobile Phase B: ACN; Flow rate: 60 mL/min; Gradient: 31% B to 46% B in 10.5 min; Wave Length: 220 nm; RT1(min): 11.2; Number Of Runs: 7) to afford 1-(1-benzyl-3-isopropyl-2-oxo-3,4-dihydroquinolin-6-yl)-3-tert-butylurea (3.3 mg, 2.46%) as a white solid. MS (ESI): mass calcd. for C₂₄H₃₁N₃O₂, 393.24 m/z, found 394.20 [M+H]⁺. ¹H NMR (300 MHz, DMSO-d6) δ 8.11 (s, 1H), 7.13-7.37 (m, 6H), 6.96 (dd, J=8.6, 2.4 Hz, 1H), 6.76 (d, J=8.7 Hz, 1H), 5.93 (s, 1H), 5.09 (d, J=3.5 Hz, 2H), 2.92 (dd, J=15.7, 5.3 Hz, 1H), 2.77 (dd, J=15.7, 8.1 Hz, 1H), 2.36 (q, J=6.9 Hz, 1H), 1.98 (p, J=6.4 Hz, 1H), 1.26 (s, 9H), 0.94 (dd, J=17.0, 6.7 Hz, 6H).

### Example 33: 3-tert-butyl-1-{4-[(3-chlorophenyl)methyl]-3-oxo-2H-1,4-benzoxazin-7-yl}urea

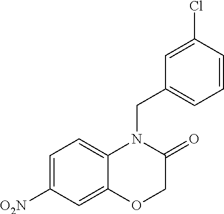 [SMILES: O=C1COc2cc([N+](=O)[O-])ccc2N1Cc1cccc(Cl)c1]

### 4-[(3-chlorophenyl)methyl]-7-nitro-2H-1,4-benzoxazin-3-one

To a solution of 7-nitro-2,4-dihydro-1,4-benzoxazin-3-one (500 mg, 2.575 mmol, 1 equiv) and 1-(bromomethyl)-3-chlorobenzene (794 mg, 3.863 mmol, 1.5 equiv) in dimethylformamide (10 mL) was added Cs₂CO₃ (2.538 g, 7.766 mmol, 3.02 equiv). The reaction was stirred at rt for 3h. Quenched with water (50 mL) and extracted with EA (3×50 mL). The combined extracts were washed with water, brine, dried over Na₂SO₄, filtered, and concentrated. The residue was purified by column chromatography on silica gel with EA/PE (0-100%) to give 4-[(3-chlorophenyl)methyl]-7-nitro-2H-1,4-benzoxazin-3-one (203 mg, 24.73%) as a white solid. MS (ESI): mass calcd for C₁₅H₁₁ClN₂O₄: 318.04 m/z, found 319.00[M+H]⁺.

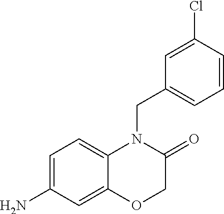 [SMILES: Nc1ccc2c(c1)OCC(=O)N2Cc1cccc(Cl)c1]

### 7-amino-4-[(3E)-4-chloro-2-methylidenebut-3-en-1-yl]-2H-1,4-benzoxazin-3-one

To a mixture of 4-[(3-chlorophenyl)methyl]-7-nitro-2H-1,4-benzoxazin-3-one (700 mg, 2.196 mmol, 1 equiv) and NH₄Cl (1.17 g, 21.960 mmol, 10 equiv) in ethyl alcohol/water (10 mL, 10/1) was added Fe (1.23 g, 21.960 mmol, 10 equiv). The reaction was stirred at 60° C. for 6h. Quenched with water (20 mL) and extracted with EA (3×20 mL). The combined extracts were washed with water, brine, dried over Na₂SO₄, filtered, and concentrated. The residue was purified by column chromatography on silica gel with EA/PE (0-100%) to give 7-amino-4-[(3E)-4-chloro-2-methylidenebut-3-en-1-yl]-2H-1,4-benzoxazin-3-one (183 mg, 31.48%) as a white solid. MS (ESI): mass calcd for C₁₅H₁₃ClN₂O₂: 288.07m/z, found 289.00[M+H]⁺.

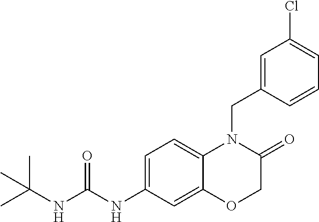 [SMILES: CC(C)(C)NC(=O)Nc1ccc2c(c1)OCC(=O)N2Cc1cccc(Cl)c1]

### 3-tert-butyl-1-{4-[(3-chlorophenyl)methyl]-3-oxo-2H-1,4-benzoxazin-7-yl}urea

To a solution of 7-amino-4-[(3-chlorophenyl)methyl]-2H-1,4-benzoxazin-3-one (200 mg, 0.693 mmol, 1 equiv) and 2-isocyanato-2-methylpropane (103.00 mg, 1.039 mmol, 1.5 equiv) in DCM (2 mL) was added TEA (210.29 mg, 2.079 mmol, 3 equiv). The reaction was stirred at rt for 3h. Quenched with water (10 mL) and extracted with EA (3×10 mL). The combined extracts were washed with water, brine, dried over Na₂SO₄, filtered, and concentrated. The residue was purified by preparative HPLC using a XBridge Prep OBD C18 Column 150 mm×30 mm×5 μm (eluent: 17% to 42% (v/v) CH₃CN and H₂O with 10 mmol/L NH₄HCO₃) to afford the title compound 3-tert-butyl-1-{4-[(3-chlorophenyl)methyl]-3-oxo-2H-1,4-benzoxazin-7-yl}urea (45.7 mg, 16.97%). LC/MS (ESI): mass calcd. for C₂₀H₂₂ClN₃O₃:387.13 m/z, found:387.95 [M+H]⁺. ¹H NMR (400 MHz, DMSO-d6) δ 8.23 (s, 1H), 7.29-7.41 (m, 3H), 7.22 (m, 2H), 6.86-6.85 (m, 2H), 5.94 (s, 1H), 5.11 (s, 2H), 4.76 (s, 2H), 1.26 (s, 9H).

### Example 34 and Example 35: (R)-1-(tert-butyl)-3-(3-oxo-4-(1-phenylethyl)-3,4-dihydro-2H-benzo[b][1,4]oxazin-7-yl)urea and (S)-1-(tert-butyl)-3-(3-oxo-4-(1-phenylethyl)-3,4-dihydro-2H-benzo[b][1,4]oxazin-7-yl)urea

### Synthetic Scheme

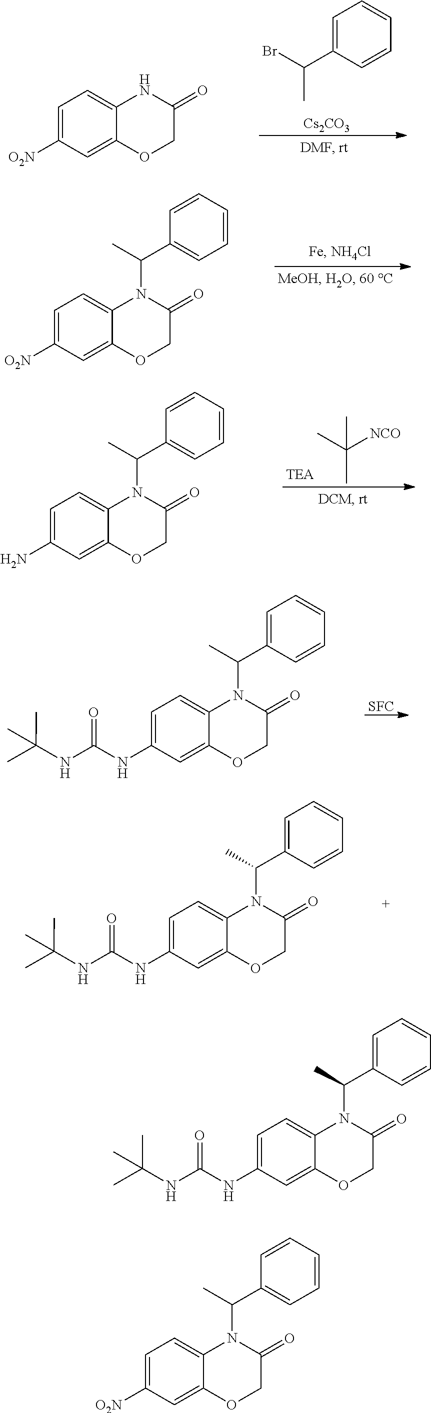

### 7-nitro-4-(1-phenylethyl)-2H-benzo[b][1,4]oxazin-3(4H)-one

To a solution of 7-nitro-2,4-dihydro-1,4-benzoxazin-3-one (500 mg, 2.575 mmol, 1 equiv) in DMF (20 mL) was added Cs₂CO₃ (1683.41 mg, 5.150 mmol, 2 equiv) and (1-bromoethyl)benzene (714.92 mg, 3.863 mmol, 1.5 equiv). The reaction was stirred at rt for 3 hours. Quenched with water (25 mL) and extracted with EA (3×25 mL). The combined extracts were washed with water, brine, dried over Na₂SO₄, filtered, and concentrated. The residue was purified by column chromatography on silica gel with EA/PE (0-100%) to give 7-nitro-4-(1-phenylethyl)-2H-1,4-benzoxazin-3-one (564 mg, 73.42%) as a yellow liquid. MS (ESI): mass calcd. for C₁₆H₁₄N₂O₄: 298.30m/z, found 299.00 [M+H]f.

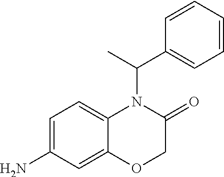 [SMILES: CC(c1ccccc1)N1C(=O)COc2cc(N)ccc21]

### 7-amino-4-(1-phenylethyl)-2H-benzo[b][1,4]oxazin-3(4H)-one

To a solution of 6-nitro-1-(1-phenylethyl)-3,4-dihydroquinolin-2-one (560 mg, 1.890 mmol, 1 equiv) in MeOH/H₂O (22 mL, 10:1) was added Fe (527.68 mg, 9.450 mmol, 5 equiv) and NH₄Cl (505.43 mg, 9.450 mmol, 5 equiv). The reaction was stirred at 80° C. for 1 hour. The reaction was quenched with water at rt. The resulting mixture was filtered, the filter cake was washed with EA (3×10 mL). The filtrate was added water (20 mL) and extracted with EA (3×20 mL). The combined extracts were washed with water, brine, dried over Na₂SO₄, filtered, and concentrated. The residue was purified by column chromatography on silica gel with EA/PE (0-100%) to give 6-amino-1-(1-phenylethyl)-3,4-dihydroquinolin-2-one (382 mg, 75.89%) as a yellow oil. MS (ESI): mass calcd. for C₁₆H₁₆N₂O₂: 268.32m/z, found 269.15 [M+H]⁺.

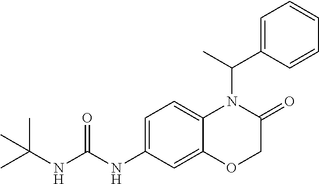 [SMILES: CC(c1ccccc1)N1C(=O)COc2cc(NC(=O)NC(C)(C)C)ccc21]

### 1-(tert-butyl)-3-(3-oxo-4-(1-phenylethyl)-3,4-dihydro-2H-benzo[b][1,4]oxazin-7-yl)urea

To a solution of 6-amino-1-[(3-fluorophenyl)methyl]-3,4-dihydroquinolin-2-one (380 mg, 1.406 mmol, 1 equiv) in DCM (15 mL) was added 2-isocyanato-2-methylpropane (557.45 mg, 5.624 mmol, 4 equiv) and TEA (426.77 mg, 4.218 mmol, 3 equiv) at 0° C. The reaction was stirred at rt for 1 days. Quenched with water (10 mL) and extracted with EA (3×10 mL). The combined extracts were washed with water, brine, dried over Na₂SO₄, filtered, and concentrated. The residue was purified by preparative HPLC using a XBridge Prep OBD C18 Column, 30*150 mm, 5 μm column (eluent: 17% to 42% (v/v) CH₃CN and H₂O with 10 mmol/L NH₄HCO₃) to afford the title compound 3-tert-butyl-1-{1-[(3-fluorophenyl)methyl]-2-oxo-3,4-dihydroquinolin-6-yl}urea (120 mg, 23.11%). LC/MS (ESI): mass calcd. for C₂₁H₂₅N₃O₃:367.45 m/z, found:368.00 [M+H]⁺.

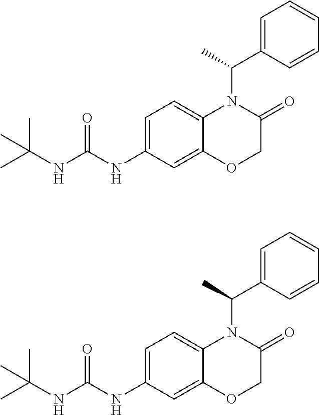 [SMILES: C[C@H](c1ccccc1)N1C(=O)COc2cc(NC(=O)NC(C)(C)C)ccc21 ; C[C@@H](c1ccccc1)N1C(=O)COc2cc(NC(=O)NC(C)(C)C)ccc21]

### (R)-1-(tert-butyl)-3-(3-oxo-4-(1-phenylethyl)-3,4-dihydro-2H-benzo[b][1,4]oxazin-7-yl)urea and (S)-1-(tert-butyl)-3-(3-oxo-4-(1-phenylethyl)-3,4-dihydro-2H-benzo[b][1,4]oxazin-7-yl)urea

A sample of 3-tert-butyl-1-[3-oxo-4-(1-phenylethyl)-2H-1,4-benzoxazin-7-yl]urea (120 mg) was separated by chiral-HPLC using a CHIRAL ART Amylose-SA, 2*25 cm, 5 μm column (eluent: 30% to 30% (v/v) Hex(0.5% 2M NH₃-MeOH) and EtOH) to yield first enantiomer 3-tert-butyl-1-{3-oxo-4-[(1R)-1-phenylethyl]-2H-1,4-benzoxazin-7-yl}urea (37.1 mg) as a white solid. MS(ESI): mass calcd. for C₂₁H₂₅N₃O₃: 367.45, found: 368.20 [M+H]⁺. ¹H NMR (400 MHz, DMSO-d₆) δ 7.99-8.31 (m, 1H), 7.11-7.44 (m, 6H), 6.43-6.67 (m, 2H), 6.04-6.24 (m, 1H), 5.80-5.98 (m, 1H), 4.55-4.80 (m, 2H), 1.67-1.80 (m, 3H), 1.22-1.31 (m, 9H). And to yield second enantiomer 3-tert-butyl-1-{3-oxo-4-[(1S)-1-phenylethyl]-2H-1,4-benzoxazin-7-yl}urea (22.7 mg) as a white solid. MS(ESI): mass calcd. for C₂₁H₂₅N₃O₃: 367.45, found: 368.20 [M+H]⁺. ¹H NMR (400 MHz, DMSO-d6) δ 8.10-8.26 (m, 1H), 7.17-7.40 (m, 6H), 6.45-6.70 (m, 2H), 6.02-6.21 (m, 1H), 5.85-6.00 (m, 1H), 4.60-4.70 (m, 2H), 1.70-1.80 (m, 3H), 1.20-1.30 (m, 9H).

### Example 36 and Example 37: (R)-1-(tert-butyl)-3-(4-(1-(3-chlorophenyl)ethyl)-3-oxo-3,4-di hydro-2H-benzo[b][1,4]oxazin-7-yl)urea and (S)-1-(tert-butyl)-3-(4-(1-(3-chlorophenyl)ethyl)-3-oxo-3,4-dihydro-2H-benzo[b][1,4]oxazin-7-yl)urea

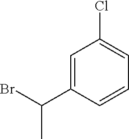

### 1-(1-bromoethyl)-3-chlorobenzene

To a solution of 1-(3-chlorophenyl)ethanol (1.5 g, 9.578 mmol, 1 equiv) in Et2O (10 mL) was added PBr₃ (5.19 g, 19.156 mmol, 2 equiv) at 0° C. The reaction was stirred at rt for 2 hours. Quenched with water (10 mL) and extracted with EA (3×10 mL). The combined extracts were washed with water, brine, dried over Na₂SO₄, filtered, and concentrated. This resulted in 1-(1-bromoethyl)-3-chlorobenzene (762 mg, 36.24%) as a white oil. MS (ESI): mass calcd. for C₈H₈BrCl: 219.51 m/z, found 220.95 [M+H]⁺.

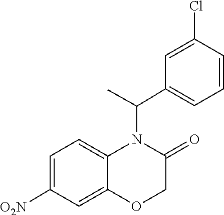 [SMILES: CC(c1cccc(Cl)c1)N1C(=O)COc2cc([N+](=O)[O-])ccc21]

### 4-(1-(3-chlorophenyl)ethyl)-7-nitro-2H-benzo[b][1,4]oxazin-3(4H)-one

To a solution of 7-nitro-2,4-dihydro-1,4-benzoxazin-3-one (500 mg, 2.575 mmol, 1 equiv) in DMF (15 mL) was added Cs₂CO₃ (1683.4 mg, 5.151 mmol, 2 equiv) and 1-(1-bromoethyl)-3-chlorobenzene (565.2 mg, 2.575 mmol, 1 equiv). The final reaction mixture was stirred for 2 hours at rt. Quenched with water (25 mL) and extracted with EA (3×25 mL). The combined extracts were washed with water, brine, dried over Na₂SO₄, filtered, and concentrated. The residue was purified by column chromatography on silica gel with EA/PE (0-100%) to give 4-[1-(3-chlorophenyl)ethyl]-7-nitro-2H-1,4-benzoxazin-3-one (560 mg, 73.40%) as a yellow solid. MS (ESI): mass calcd. for C₁₆H₁₃ClN₂O₄: 332.74 m/z, found 333.00 [M+H]⁺.

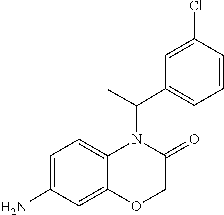 [SMILES: CC(c1cccc(Cl)c1)N1C(=O)COc2cc(N)ccc21]

### 7-amino-4-(1-(3-chlorophenyl)ethyl)-2H-benzo[b][1,4]oxazin-3(4H)-one

To a solution of 1-[1-(3-chlorophenyl)ethyl]-6-nitro-3,4-dihydroquinolin-2-one (560 mg, 1.693 mmol, 1 equiv) in MeOH/H₂O (22 mL, 10:1) was added Fe (472.73 mg, 8.465 mmol, 5 equiv) and NH₄Cl (452.80 mg, 8.465 mmol, 5 equiv). The reaction was stirred at 80° C. for 1 hour. The reaction was stirred for 1 hour at 80° C. The reaction was quenched with water at rt. After filtration, the filter cake was washed with EA (3×20 mL). The filtrate was added water (20 mL) and extracted with EA (3×20 mL). The combined extracts were washed with water, brine, dried over Na₂SO₄, filtered, and concentrated. The residue was purified by column chromatography on silica gel with EA/PE (0-100%) to give 6-amino-1-[1-(3-chlorophenyl)ethyl]-3,4-dihydroquinolin-2-one (470 mg, 92.29%) as a yellow oil. MS (ESI): mass calcd. for C₁₆H₁₅ClN₂O₂: 302.76 m/z, found:303.00 [M+H]⁺.

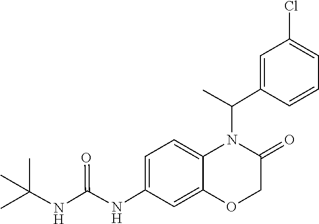 [SMILES: CC(c1cccc(Cl)c1)N1C(=O)COc2cc(NC(=O)NC(C)(C)C)ccc21]

### 1-(tert-butyl)-3-(4-(1-(3-chlorophenyl)ethyl)-3-oxo-3,4-dihydro-2H-benzo[b][1,4]oxazin-7-yl)urea

To a solution of 6-amino-1-[1-(3-chlorophenyl)ethyl]-3,4-dihydroquinolin-2-one (470 mg, 1.563 mmol, 1 equiv) in DCM (15 mL) was added 2-isocyanato-2-methylpropane (619.60 mg, 6.252 mmol, 4 equiv) and TEA (474.36 mg, 4.689 mmol, 3 equiv) at 0° C. The reaction was stirred at rt for 1 day. Quenched with water (10 mL) and extracted with EA (3×10 mL). The combined extracts were washed with water, brine, dried over Na₂SO₄, filtered, and concentrated. The residue was purified by preparative HPLC using a YMC-Actus Triart C18 ExRS, 30*150 mm, 5 μm column (eluent: 49% to 69% (v/v) CH₃CN and H₂O with 10 mmol/L NH₄HCO₃) to afford the title compound 3-tert-butyl-1-{1-[1-(3-chlorophenyl)ethyl]-2-oxo-3,4-dihydroquinolin-6-yl}urea (470 mg, 74.8%) as a white solid. MS (ESI): mass calcd for C₂₁H₂₄ClN₃O₃:401.89 m/z, found:402.00 [M+H]⁺.

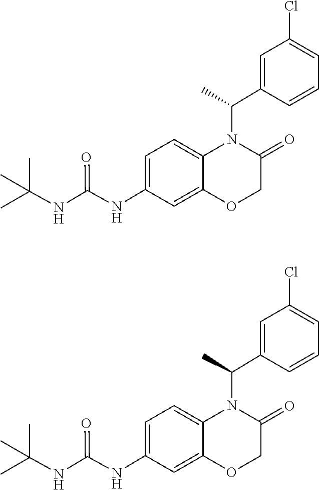 [SMILES: C[C@H](c1cccc(Cl)c1)N1C(=O)COc2cc(NC(=O)NC(C)(C)C)ccc21 ; C[C@@H](c1cccc(Cl)c1)N1C(=O)COc2cc(NC(=O)NC(C)(C)C)ccc21]

### (R)-1-(tert-butyl)-3-(4-(1-(3-chlorophenyl)ethyl)-3-oxo-3,4-dihydro-2H-benzo[b][1,4]oxazin-7-yl)urea and (S)-1-(tert-butyl)-3-(4-(1-(3-chlorophenyl)ethyl)-3-oxo-3,4-dihydro-2H-benzo[b][1,4]oxazin-7-yl)urea

A sample of 3-tert-butyl-1-{4-[1-(3-chlorophenyl)ethyl]-3-oxo-2H-1,4-benzoxazin-7-yl}urea (200 mg) was separated by chiral-HPLC using a CHIRALPAK IG, 2*25 cm, 5 μm column (eluent: 30% to 30% (v/v) Hex(0.5% 2M NH₃-MeOH) and EtOH) to yield first enantiomer example 3-tert-butyl-1-{4-[(1R)-1-(3-chlorophenyl)ethyl]-3-oxo-2H-1,4-benzoxazin-7-yl}urea (48.0 mg) as a white solid. LC/MS: mass calcd. for C₂₁H₂₄ClN₃O₃: 401.89, found:402.15 [M+H]⁺. ¹H NMR (400 MHz, DMSO-d6) δ 8.10-8.30 (m, 1H), 7.30-7.44 (m, 3H), 7.20-7.30 (m, 2H), 6.51-6.72 (m, 2H), 5.99-6.16 (m, 1H), 5.81-5.99 (m, 1H), 4.59-4.78 (m, 2H), 1.68-1.78 (m, 3H), 1.24-1.29 (m, 9H). And to yield second enantiomer example 3-tert-butyl-1-{4-[(1S)-1-(3-chlorophenyl)ethyl]-3-oxo-2H-1,4-benzoxazin-7-yl}urea (26.2 mg) as a white solid. MS(ESI): mass calcd. for C₂₁H₂₄ClN₃O₃: 401.89, found:402.15 [M+H]⁺. ¹H NMR (400 MHz, DMSO-d6) δ 7.96-8.35 (m, 1H), 7.30-7.49 (m, 4H), 6.65-6.75 (m, 1H), 6.48-6.51 (m, 1H), 6.00-6.17 (m, 1H), 5.81-6.00 (m, 1H), 4.60-4.80 (m, 2H), 1.68-1.81 (m, 3H), 1.18-1.44 (m, 9H).

### Example 38: 1-(3-benzyl-2-oxo-2,3-dihydrobenzo[d]oxazol-6-yl)-3-(tert-butyl)urea

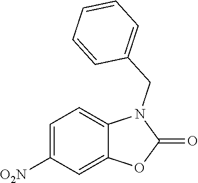 [SMILES: O=c1oc2cc([N+](=O)[O-])ccc2n1Cc1ccccc1]

### 3-benzyl-6-nitrobenzo[d]oxazol-2(3H)-one

To a solution of 6-nitrobenzo[d]oxazol-2(3H)-one (300 mg, 1.666 mmol, 1 equiv) in CH₃CN (6 mL) was added KI (276.49 mg, 1.666 mmol, 1 equiv), K₂CO₃ (788.35 mg, 5.664 mmol, 3.4 equiv) and benzyl bromide (112.4 mg, 0.657 mmol, 1.1 equiv). The resulting mixture was stirred for 5 h at 60° C. The mixture was diluted with EtOAc (100 mL), washed with water (3×30 mL), brine (2×30 mL) and concentrated under vacuum to yield a crude product which was directly purified by silica gel column, eluted with PE/EA (3:1) to afford 3-benzyl-6-nitrobenzo[d]oxazol-2(3H)-one (300 mg, 66.65%) as a yellow solid. MS (ESI): mass calcd. for C₁₄H₁₀N₂O₄270.06 m/z, found 271.05 [M+H]⁺.

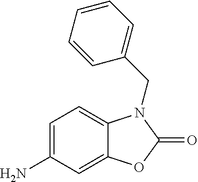 [SMILES: Nc1ccc2c(c1)oc(=O)n2Cc1ccccc1]

### 6-amino-3-benzylbenzo[d]oxazol-2(3H)-one

To a solution of 3-benzyl-6-nitrobenzo[d]oxazol-2(3H)-one (200 mg, 0.740 mmol, 1 equiv) in MeOH (10 mL) was added Pd/C (50 mg). The reaction mixture was then stirred for 1 h under hydrogen atmosphere (balloon). The reaction mixture was filtered through a pad of Celite and concentrated under reduced pressure to afford 6-amino-3-benzylbenzo[d]oxazol-2(3H)-one (150 mg, 84.36%) as a white solid. MS (ESI): mass calcd. for C₁₄H₁₂N₂O₂, 240.09 m/z, found 241.20 [M+H]⁺.

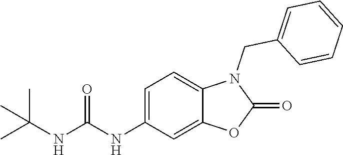 [SMILES: CC(C)(C)NC(=O)Nc1ccc2c(c1)oc(=O)n2Cc1ccccc1]

### 1-(3-benzyl-2-oxo-2,3-dihydrobenzo[d]oxazol-6-yl)-3-(tert-butyl)urea

To a solution of 6-amino-3-benzylbenzo[d]oxazol-2(3H)-one (100 mg, 0.416 mmol, 1 equiv) in DCM (8 mL) was added TEA (126.3 mg, 1.249 mmol, 3 equiv) and 2-isocyanato-2-methylpropane (123.7 mg, 1.249 mmol, 3 equiv). The resulting mixture was stirred for 16 h at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 1-(3-benzyl-2-oxo-2,3-dihydrobenzo[d]oxazol-6-yl)-3-(tert-butyl)urea (12.8 mg, 9.03%) as a off-white solid. MS (ESI): mass calcd. for C₁₉H₂₁N₃O₃, 339.16 m/z, found 340.10 [M+H]⁺. ¹H NMR (300 MHz, DMSO-d6) δ 8.30 (s, 1H), 7.61 (d, J=1.9 Hz, 1H), 7.27-7.39 (m, 5H), 7.01 (d, J 8.5 Hz, 1H), 6.89 (dd, J=8.5, 2.0 Hz, 1H), 5.95 (s, 1H), 4.99 (s, 2H), 1.27 (s, 9H).

### Example 39: 1-(tert-butyl)-3-(1-(2-fluoro-5-(trifluoromethyl)benzyl)-2-oxo-1,2,3,4-tetrahydroquinolin-6-yl)urea

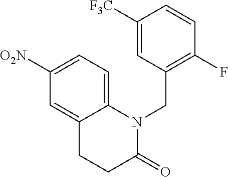 [SMILES: O=C1CCc2cc([N+](=O)[O-])ccc2N1Cc1cc(C(F)(F)F)ccc1F]

### 1-(2-fluoro-5-(trifluoromethyl)benzyl)-6-nitro-3,4-dihydroquinolin-2(1H)-one

To a solution of 6-nitro-3,4-dihydroquinolin-2(1H)-one (500 mg, 2.602 mmol, 1 equiv) in DMF (10 mL) was added Cs₂CO₃ (1.695 g, 5.20 mmol, 2 equiv) and 2-(bromomethyl)-1-fluoro-4-(trifluoromethyl)benzene (735.1 mg, 2.860 mmol, 1.1 equiv). The resulting mixture was stirred for 16 h at rt. The mixture was diluted with EtOAc (100 mL), washed with water (3×30 mL), brine (2×30 mL) and concentrated under vacuum to yield a crude product which was directly purified by silica gel column, eluted with PE/EA (3:1) to afford 7-nitro-4-(3-(trifluoromethyl)benzyl)-2H-benzo[b][1,4]oxazin-3(4H)-one (533.4 mg, 55.66%) as a white solid. MS (ESI): mass calcd. for C₁₇H₁₂F₄N₂O₃: 368.08 m/z, found 369.10 [M+H]⁺.

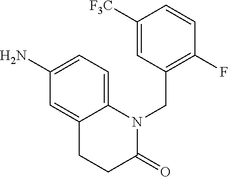 [SMILES: Nc1ccc2c(c1)CCC(=O)N2Cc1cc(C(F)(F)F)ccc1F]

### 6-amino-1-(2-fluoro-5-(trifluoromethyl)benzyl)-3,4-dihydroquinolin-2(1H)-one

To a solution of 7-nitro-4-(3-(trifluoromethyl)benzyl)-2H-benzo[b][1,4]oxazin-3(4H)-one (200 mg, 0.543 mmol, 1 equiv) in MeOH (10 mL) was added Pd/C (50 mg). The reaction mixture was then stirred for 1 h under hydrogen atmosphere (balloon). The reaction mixture was filtered through a pad of Celite and concentrated under reduced pressure to afford 6-amino-1-(2-fluoro-5-(trifluoromethyl)benzyl)-3,4-dihydroquinolin-2(1H)-one (130 mg, 71.0%) as a white solid. MS (ESI): mass calcd. for C₁₇H₁₄F₄N₂O, 338.10 m/z, found 339.05 [M+H]⁺.

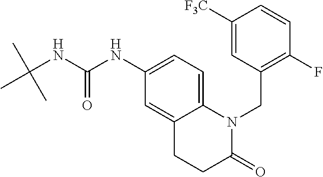 [SMILES: CC(C)(C)NC(=O)Nc1ccc2c(c1)CCC(=O)N2Cc1cc(C(F)(F)F)ccc1F]

### 1-(tert-butyl)-3-(1-(2-fluoro-5-(trifluoromethyl)benzyl)-2-oxo-1,2,3,4-tetrahydroquinolin-6-yl)urea

To a stirred solution of 6-amino-1-(2-fluoro-5-(trifluoromethyl)benzyl)-3,4-dihydroquinolin-2(1H)-one (130 mg, 0.384 mmol, 1 equiv) in DCM (6 mL) & toluene (2 mL) was added 2-isocyanato-2-methylpropane (114.34 mg, 1.153 mmol, 3 equiv) stirred 16 h at 90° C. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 45% B in 9 min, 45% B; Wave Length: 254/220 nm; RT1(min): 8.9; Number Of Runs: 0) to afford 1-(tert-butyl)-3-(1-(2-fluoro-5-(trifluoromethyl)benzyl)-2-oxo-1,2,3,4-tetrahydroquinolin-6-yl)urea (22.5 mg, 13.39%) as a white solid. MS (ESI): mass calcd. for C₂₂H₂₃F₄N₃O₂, 437.17 m/z, found 438.15 [M+H]⁺. ¹H NMR (300 MHz, DMSO-d6) δ 8.15 (s, 1H), 7.66-7.78 (m, 1H), 7.48 (d, J=9.3 Hz, 1H), 7.29-7.41 (m, 2H), 7.03 (dd, J=8.7, 2.5 Hz, 1H), 6.81 (d, J=8.8 Hz, 1H), 5.94 (s, 1H), 5.18 (s, 2H), 2.81-2.96 (m, 2H), 2.60-2.71 (m, 2H), 1.26 (s, 9H).

### Example 40: 1-(tert-butyl)-3-(4-(3-methylbenzyl)-3-oxo-3,4-dihydro-2H-benzo[b][1,4]oxazin-7-yl)urea

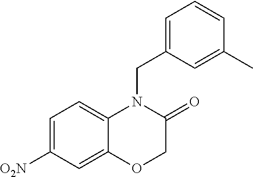 [SMILES: Cc1cccc(CN2C(=O)COc3cc([N+](=O)[O-])ccc32)c1]

### 4-(3-methylbenzyl)-7-nitro-2H-benzo[b][1,4]oxazin-3(4H)-one

To a stirred solution of 7-nitro-2,4-dihydro-1,4-benzoxazin-3-one (500 mg, 2.575 mmol, 1 equiv) in anhydrous dimethylformamide (10 mL) was added 1-(bromomethyl)-3-methylbenzene (619.59 mg, 3.348 mmol, 1.3 equiv) and Cs₂CO₃ (2.52 g, 7.725 mmol, 3 equiv) at rt and stirred for overnight. The reaction progress was monitored by LCMS. After completion of reaction, the resulting mixture was extracted with EA (3×30 mL). The combined organic layers were washed with brine (1×30 mL), dried over anhydrous Na₂SO₄. After filtration, the filtrate was concentrated under reduced pressure. The residue was purified by silica gel column chromatography (PE/EA=3:1) to afford 4-[(3-methylphenyl)methyl]-7-nitro-2H-1,4-benzoxazin-3-one (750 mg, 97.63%) as a yellow solid. MS (ESI): mass calcd. for C₁₆H₁₄N₂O₄: 298.10 m/z, found 299.05 [M+H]⁺.

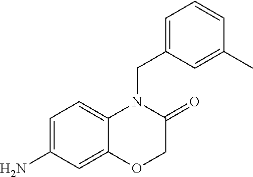 [SMILES: Cc1cccc(CN2C(=O)COc3cc(N)ccc32)c1]

### 7-amino-4-(3-methylbenzyl)-2H-benzo[b][1,4]oxazin-3(4H)-one

To a solution of 4-[(3-methylphenyl)methyl]-7-nitro-2H-1,4-benzoxazin-3-one (300 mg, 1.006 mmol, 1 equiv) in 20 mL MeOH was added Pd/C (10%, 150 mg) under nitrogen atmosphere in a 50 mL bar. The mixture was hydrogenated at room temperature for 30 min under hydrogen atmosphere using a hydrogen balloon, filtered through a Celite pad and concentrated under reduced pressure. This resulted in 7-amino-4-[(3-methylphenyl)methyl]-2H-1,4-benzoxazin-3-one (250 mg, 92.65%) as a white solid. MS (ESI): mass calcd. for C₁₆H₁₆N₂O₂: 268.12 m/z, found 269.10 [M+H]⁺.

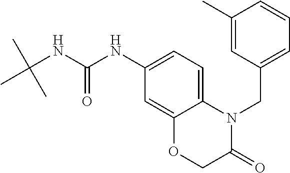 [SMILES: Cc1cccc(CN2C(=O)COc3cc(NC(=O)NC(C)(C)C)ccc32)c1]

### 1-(tert-butyl)-3-(4-(3-methylbenzyl)-3-oxo-3,4-dihydro-2H-benzo[b][1,4]oxazin-7-yl)urea

To a stirred solution of 7-amino-4-[(3-methylphenyl)methyl]-2H-1,4-benzoxazin-3-one (50 mg, 0.186 mmol, 1 equiv) in DCM (5 mL) and Toluene (2.5 mL) was added 2-isocyanato-2-methylpropane (55.42 mg, 0.558 mmol, 3 equiv) at 90° C. and stirred for overnight. The reaction progress was monitored by TLC. After completion of reaction, the reaction mixture was concentrated under reduced pressure to give crude product which was further purified by HPLC with the following conditions Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0 to afford 3-tert-butyl-1-{4-[(3-methylphenyl)methyl]-3-oxo-2H-1,4-benzoxazin-7-yl}urea (2.6 mg, 3.74%) as a white solid. MS (ESI):mass calcd. for C21H25N3O3: 367.19 m/z, found 368.20 [M+H]⁺. ¹H NMR (300 MHz, DMSO-d6) δ 8.20 (s, 1H), 7.19 (d, J=6.2 Hz, 2H), 7.06 (d, J=7.3 Hz, 3H), 6.85 (d, J=8.6 Hz, 1H), 6.75 (d, J=8.8 Hz, 1H), 5.93 (s, 1H), 5.06 (s, 2H), 4.74 (s, 2H), 2.27 (s, 3H), 1.26 (s, 9H).

### Example 41 and Example 42: (R)-1-(4-benzyl-2-methyl-3-oxo-3,4-dihydro-2H-benzo[b][1,4]oxazin-7-yl)-3-(tert-butyl)urea and (S)-1-(4-benzyl-2-methyl-3-oxo-3,4-dihydro-2H-benzo[b][1,4]oxazin-7-yl)-3-(tert-butyl)urea

### Synthetic Scheme

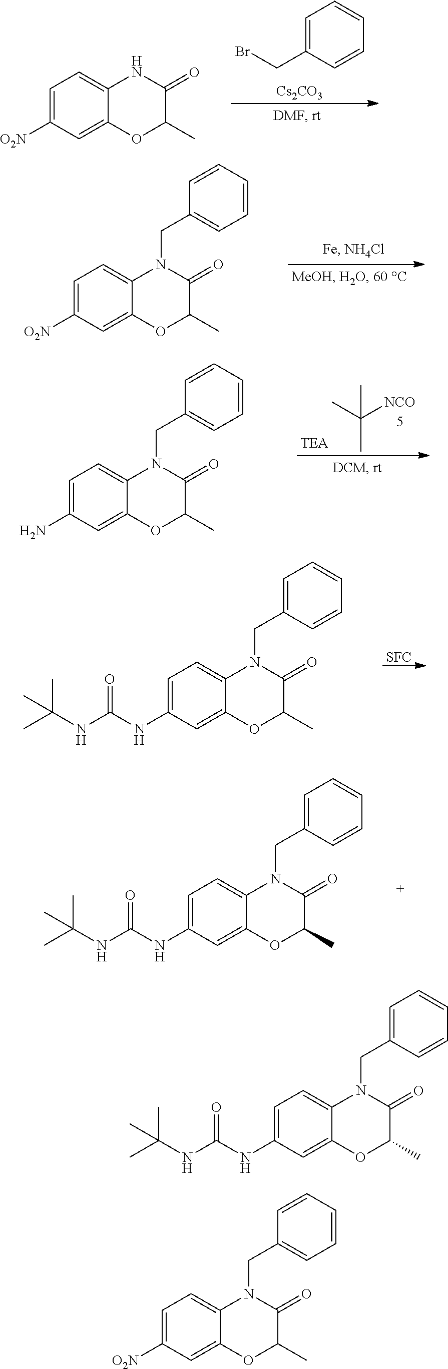

### 4-benzyl-2-methyl-7-nitro-2H-benzo[b][1,4]oxazin-3(4H)-one

To a solution of 2-methyl-7-nitro-2,4-dihydro-1,4-benzoxazin-3-one (300 mg, 1.441 mmol, 1 equiv) in DMF (10 mL) was added Cs₂CO₃ (941.99 mg, 2.882 mmol, 2 equiv) and benzyl bromide (369.72 mg, 2.162 mmol, 1.5 equiv). The reaction was stirred at rt for 1 hour. Quenched with water (10 mL) and extracted with EA (3×10 mL). The combined extracts were washed with water, brine, dried over Na₂SO₄, filtered, and concentrated. The residue was purified by column chromatography on silica gel with EA/PE (0-100%) to give 4-benzyl-2-methyl-7-nitro-2H-1,4-benzoxazin-3-one (377 mg, 87.70%) as a yellow solid. MS (ESI): mass calcd. for C₁₆H₁₄N₂O₄: 298.30m/z, found 299.00 [M+H]⁺.

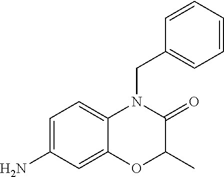 [SMILES: CC1Oc2cc(N)ccc2N(Cc2ccccc2)C1=O]

### 7-amino-4-benzyl-2-methyl-2H-benzo[b][1,4]oxazin-3(4H)-one

To a solution of 6-nitro-1-(1-phenylethyl)-3,4-dihydroquinolin-2-one (560 mg, 1.890 mmol, 1 equiv) in MeOH/H₂O (22 mL, 10:1) was added Fe (527.68 mg, 9.450 mmol, 5 equiv) and NH₄Cl (505.43 mg, 9.450 mmol, 5 equiv). The reaction was stirred at 80° C. for 1 hour. The reaction was quenched with water at rt. After filtration, the filter cake was washed with EA (3×10 mL). The filtrate was added water (20 mL) and extracted with EA (3×20 mL). The combined extracts were washed with water, brine, dried over Na₂SO₄, filtered, and concentrated. The residue was purified by column chromatography on silica gel with EA/PE (0-100%) to give 7-amino-4-benzyl-2-methyl-2H-1,4-benzoxazin-3-one (530 mg, 99.87%) as a yellow oil. MS (ESI): mass calcd. for C₁₆H₁₆N₂O₂: 268.32m/z, found 269.05 [M+H]+.

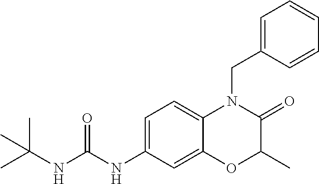 [SMILES: CC1Oc2cc(NC(=O)NC(C)(C)C)ccc2N(Cc2ccccc2)C1=O]

### 1-(4-benzyl-2-methyl-3-oxo-3,4-dihydro-2H-benzo[b][1,4]oxazin-7-yl)-3-(tert-butyl)urea

To a solution of 7-amino-4-benzyl-2-methyl-2H-1,4-benzoxazin-3-one (540 mg, 2.013 mmol, 1 equiv) in DCM/Toluene (15 mL, 2:1) was added 2-isocyanato-2-methylpropane (798.04 mg, 8.052 mmol, 4 equiv). The reaction was stirred at 90° C. for 2 hours. Quenched with water (20 mL) and extracted with EA (3×20 mL). The combined extracts were washed with water, brine, dried over Na₂SO₄, filtered, and concentrated. The residue was purified by column chromatography on silica gel with EA/PE (0-100%) to give 1-(4-benzyl-2-methyl-3-oxo-2H-1,4-benzoxazin-7-yl)-3-tert-butylurea (498 mg, 67.34%) as a white solid. MS (ESI): mass calcd. for C₂₁H₂₅N₃O₃:367.45 m/z, found:368.10 [M+H]⁺.

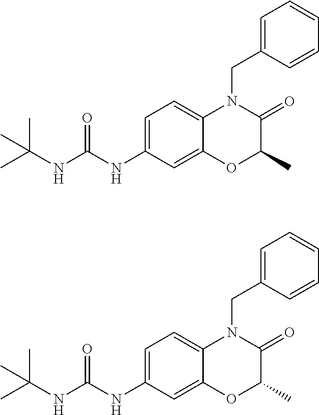 [SMILES: C[C@H]1Oc2cc(NC(=O)NC(C)(C)C)ccc2N(Cc2ccccc2)C1=O ; C[C@@H]1Oc2cc(NC(=O)NC(C)(C)C)ccc2N(Cc2ccccc2)C1=O]

### (R)-1-(4-benzyl-2-methyl-3-oxo-3,4-dihydro-2H-benzo[b][1,4]oxazin-7-yl)-3-(tert-butyl)urea and (S)-1-(4-benzyl-2-methyl-3-oxo-3,4-dihydro-2H-benzo[b][1,4]oxazin-7-yl)-3-(tert-butyl)urea

A sample of 1-(4-benzyl-2-methyl-3-oxo-2H-1,4-benzoxazin-7-yl)-3-tert-butylurea (200 mg) was separated by chiral-HPLC using a CHIRALPAK IE, 2*25 cm, 5 μm column (eluent: 15% to 15% (v/v) Hex(0.5% 2M NH₃-MeOH) and EtOH) to yield first enantiomer example 1-[(2R)-4-benzyl-2-methyl-3-oxo-2H-1,4-benzoxazin-7-yl]-3-tert-butylurea (29.8 mg) as a white solid. MS(ESI): mass calcd. for C₂₁H₂₅N₃O₃: 367.45, found: 368.15 [M+H]⁺. ¹H NMR (400 MHz, DMSO-d6) δ 8.14-8.32 (m, 1H), 7.15-7.45 (m, 6H), 6.68-6.98 (m, 2H), 5.79-6.09 (m, 1H), 5.00-5.17 (m, 2H), 4.70-4.90 (m, 1H), 1.43-1.65 (m, 3H), 1.18-1.31 (m, 9H). And to yield second enantiomer example 1-[(2S)-4-benzyl-2-methyl-3-oxo-2H-1,4-benzoxazin-7-yl]-3-tert-butylurea (25.5 mg) as a white solid. MS(ESI): mass calcd. for C₂₁H₂₅N₃O₃: 367.45, found: 368.15 [M+H]⁺. ¹H NMR (400 MHz, DMSO-d6) δ 8.08-8.31 (m, 1H), 7.30-7.39 (m, 2H), 7.18-7.30 (m, 4H), 6.71-6.92 (m, 2H), 5.75-6.07 (m, 1H), 5.01-5.19 (m, 2H), 4.72-4.87 (m, 1H), 1.42-1.53 (m, 3H), 1.21-1.33 (m, 9H).

### Example 43 and Example 44: (R)-1-(tert-butyl)-3-(4-(3-chlorobenzyl)-2-methyl-3-oxo-3,4-di hydro-2H-benzo[b][1,4]oxazin-7-yl)urea and (S)-1-(tert-butyl)-3-(4-(3-chlorobenzyl)-2-methyl-3-oxo-3,4-dihydro-2H-benzo[b][1,4]oxazin-7-yl)urea

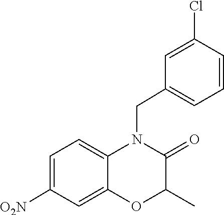 [SMILES: CC1Oc2cc([N+](=O)[O-])ccc2N(Cc2cccc(Cl)c2)C1=O]

### 4-(3-chlorobenzyl)-2-methyl-7-nitro-2H-benzo[b][1,4]oxazin-3(4H)-one

To a solution of 2-methyl-7-nitro-2,4-dihydro-1,4-benzoxazin-3-one (500 mg, 2.402 mmol, 1 equiv) in DMF (10 mL) was added Cs₂CO₃ (1569.98 mg, 4.804 mmol, 2 equiv) and 1-(bromomethyl)-3-chlorobenzene (740.30 mg, 3.603 mmol, 1.5 equiv). The reaction was stirred at rt for 1 hour. Quenched with water (10 mL) and extracted with EA (3×10 mL). The combined extracts were washed with water, brine, dried over Na₂SO₄, filtered, and concentrated. The residue was purified by column chromatography on silica gel with EA/PE (0-100%) to give 4-[(3-chlorophenyl)methyl]-2-methyl-7-nitro-2H-1,4-benzoxazin-3-one (790 mg, 98.85%) as a yellow solid. MS (ESI): mass calcd. for C₁₆H₁₃ClN₂O₄: 332.74 m/z, found 332.95 [M+H]⁺.

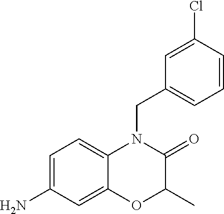 [SMILES: CC1Oc2cc(N)ccc2N(Cc2cccc(Cl)c2)C1=O]

### 7-amino-4-(3-chlorobenzyl)-2-methyl-2H-benzo[b][1,4]oxazin-3(4H)-one

To a solution of 4-[(3-chlorophenyl)methyl]-2-methyl-7-nitro-2H-1,4-benzoxazin-3-one (570 mg, 1.713 mmol, 1 equiv) in EtOH (20 mL) was added SnCl₂ (1641.36 mg, 8.565 mmol, 5 equiv). The reaction was stirred at 60° C. for 1 hour. Quenched with water (10 mL) and extracted with EA (3×10 mL). The combined extracts were washed with water, brine, dried over Na₂SO₄, filtered, and concentrated. The residue was purified by column chromatography on silica gel with EA/PE (0-100%) to give 7-amino-4-[(3-chlorophenyl)methyl]-2-methyl-2H-1,4-benzoxazin-3-one (500 mg, 96.41%) as a yellow solid. MS (ESI): mass calcd. for C₁₆H₁₅ClN₂O₂: 302.76m/z, found 302.95 [M+H]⁺.

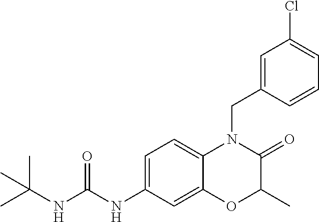 [SMILES: CC1Oc2cc(NC(=O)NC(C)(C)C)ccc2N(Cc2cccc(Cl)c2)C1=O]

### 1-(tert-butyl)-3-(4-(3-chlorobenzyl)-2-methyl-3-oxo-3,4-dihydro-2H-benzo[b][1,4]oxazin-7-yl)urea

To a solution of 7-amino-4-[(3-chlorophenyl)methyl]-2-methyl-2H-1,4-benzoxazin-3-one (500 mg, 1.651 mmol, 1 equiv) in DCM/Toluene (25 mL, 2:1) was added 2-isocyanato-2-methylpropane (654.86 mg, 6.604 mmol, 4 equiv). The reaction was stirred at 90° C. for 2 hours. Quenched with water (20 mL) and extracted with EA (3×20 mL). The combined extracts were washed with water, brine, dried over Na₂SO₄, filtered, and concentrated. The residue was purified by column chromatography on silica gel with EA/PE (0-100%) to give 3-tert-butyl-1-{4-[(3-chlorophenyl)methyl]-2-methyl-3-oxo-2H-1,4-benzoxazin-7-yl}urea (170 mg, 25.61%) as a white solid. LC/MS (ESI): mass calcd. For C₂₁H₂₄ClN₃O₃: 401.89 m/z, found:402.05 [M+H]⁺.

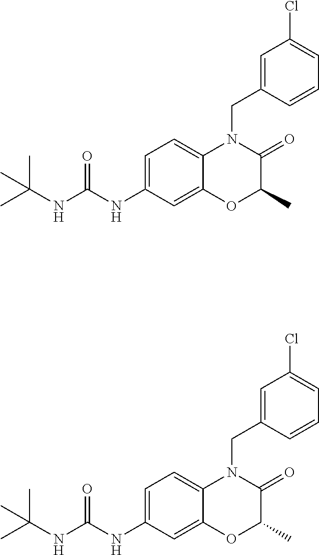 [SMILES: C[C@H]1Oc2cc(NC(=O)NC(C)(C)C)ccc2N(Cc2cccc(Cl)c2)C1=O ; C[C@@H]1Oc2cc(NC(=O)NC(C)(C)C)ccc2N(Cc2cccc(Cl)c2)C1=O]

### (R)-1-(tert-butyl)-3-(4-(3-chlorobenzyl)-2-methyl-3-oxo-3,4-dihydro-2H-benzo[b][1,4]oxazin-7-yl)urea and (S)-1-(tert-butyl)-3-(4-(3-chlorobenzyl)-2-methyl-3-oxo-3,4-dihydro-2H-benzo[b][1,4]oxazin-7-yl)urea

A sample of 3-tert-butyl-1-{2-methyl-4-[(3-methylphenyl)methyl]-3-oxo-2H-1,4-benzoxazin-7-yl}urea (170 mg) was separated by chiral-HPLC using a CHIRALPAK IE, 2*25 cm, 5 μm column (eluent: 20% to 20% (v/v) Hex(0.5% 2M NH₃-MeOH) and EtOH) to yield first enantiomer example 3-tert-butyl-1-[(2R)-2-methyl-4-[(3-methylphenyl)methyl]-3-oxo-2H-1,4-benzoxazin-7-yl]urea (23 mg) as a white solid. MS(ESI): mass calcd. for C₂₁H₂₄ClN₃O₃: 401.89, found: 402.15 [M+H]⁺. ¹H NMR (400 MHz, DMSO-d6) δ 7.93-8.31 (m, 1H), 7.29-7.44 (m, 3H), 7.13-7.29 (m, 2H), 6.68-6.99 (m, 2H), 5.84-6.08 (m, 1H), 4.97-5.21 (m, 2H), 4.71-4.89 (m, 1H), 1.40-1.58 (m, 3H), 1.21-1.30 (m, 9H). And to yield second enantiomer example 3-tert-butyl-1-[(2S)-2-methyl-4-[(3-methylphenyl)methyl]-3-oxo-2H-1,4-benzoxazin-7-yl]urea (24.5 mg) as a white solid. MS(ESI): mass calcd. for C₂₁H₂₄ClN₃O₃: 401.89, found: 402.15 [M+H]⁺. ¹H NMR (400 MHz, DMSO-d6) δ 8.00-8.32 (m, 1H), 7.30-7.49 (m, 3H), 7.19-7.30 (m, 2H), 6.70-6.92 (m, 2H), 5.81-6.06 (m, 1H), 5.00-5.21 (m, 2H), 4.66-4.91 (m, 1H), 1.43-1.54 (m, 3H), 1.25-1.30 (m, 9H).

### Example 45 and Example 45: 3-tert-butyl-1-{3-methyl-2-oxo-1-[(1S)-1-phenylethyl]-4H-quinazolin-6-yl}urea and 3-tert-butyl-1-{3-methyl-2-oxo-1-[(1R)-1-phenylethyl]-4H-quinazolin-6-yl}urea

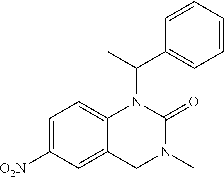 [SMILES: CC(c1ccccc1)N1C(=O)N(C)Cc2cc([N+](=O)[O-])ccc21]

### 3-methyl-6-nitro-1-(1-phenylethyl)-4H-quinazolin-2-one

To a solution of 3-methyl-6-nitro-1,4-dihydroquinazolin-2-one (600 mg, 2.896 mmol, 1 equiv) and (1-bromoethyl)benzene (800 mg, 4.323 mmol, 1.49 equiv) in dimethylformamide (30 mL) was added Cs₂CO₃ (2802.47 mg, 8.575 mmol, 2.96 equiv). The reaction was stirred at rt for 3h. Quenched with water (50 mL) and extracted with EA (3×50 mL). The combined extracts were washed with water, brine, dried over Na₂SO₄, filtered, and concentrated. The residue was purified by column chromatography on silica gel with EA/PE (0-100%) to give 3-methyl-6-nitro-1-(1-phenylethyl)-4H-quinazolin-2-one (765 mg, 84.85%) as a white solid. MS (ESI): mass calcd for C₁₇H₁₉N₃O₂: 311.13 m/z, found 312.20[M+H].

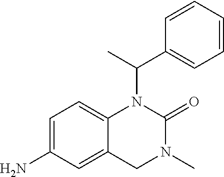 [SMILES: CC(c1ccccc1)N1C(=O)N(C)Cc2cc(N)ccc21]

### 6-amino-3-methyl-1-(1-phenylethyl)-4H-quinazolin-2-one

To a solution of 3-methyl-6-nitro-1-(1-phenylethyl)-4H-quinazolin-2-one (755 mg, 2.425 mmol, 1 equiv) in ethyl alcohol/water (10 mL, 10/1) was added iron (1354.24 mg, 24.250 mmol, 10 equiv) and NH₄Cl (1297.13 mg, 24.250 mmol, 10 equiv). The reaction was stirred at 80° C. for 3h. Quenched with water (30 mL) and extracted with EA (3×30 mL). The combined extracts were washed with water, brine, dried over Na₂SO₄, filtered, and concentrated. The residue was purified by column chromatography on silica gel with EA/PE (0-100%) to 6-amino-3-methyl-1-(1-phenylethyl)-4H-quinazolin-2-one (200 mg, 29.31%) as a white solid. MS (ESI): mass calcd for C₁₇H₁₉N₃O: 281.15m/z, found 282.20 [M+H]⁺.

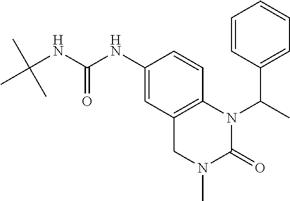 [SMILES: CC(c1ccccc1)N1C(=O)N(C)Cc2cc(NC(=O)NC(C)(C)C)ccc21]

### 1-(tert-butyl)-3-(3-methyl-2-oxo-1-(1-phenylethyl)-1,2,3,4-tetrahydroquinazolin-6-yl)urea

To a solution of 6-amino-3-methyl-1-(1-phenylethyl)-4H-quinazolin-2-one (190 mg, 0.675 mmol, 1 equiv) in DCM/toluene (5 mL2/1) was added 2-isocyanato-2-methylpropane (220.64 mg, 2.226 mmol, 3.30 equiv) The reaction was stirred at 90° C. for 3h. Quenched with water (10 mL) and extracted with DCM (3×10 mL). The combined extracts were washed with water, brine, dried over Na₂SO₄, filtered, and concentrated. The residue was purified by column chromatography on silica gel with EA/PE (0-100%) to give 3-tert-butyl-1-{3-methyl-2-oxo-1-[(1R)-1-phenylethyl]-4H-quinazolin-6-yl}urea (178 mg, 69.3%) as a white solid LC/MS (ESI): mass calcd. for C₂₂H₂₈N₄O₂:380.22 m/z, found:381.10 [M+H]⁺.

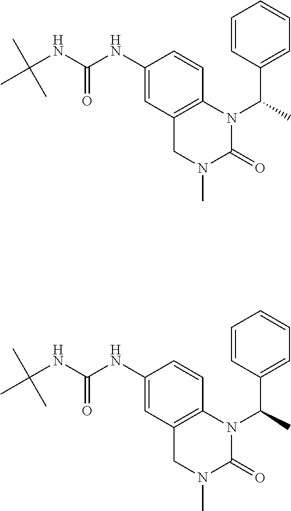

### (S)-1-(tert-butyl)-3-(3-methyl-2-oxo-1-(1-phenylethyl)-1,2,3,4-tetrahydroquinazolin-6-yl)urea and (R)-1-(tert-butyl)-3-(3-methyl-2-oxo-1-(1-phenylethyl)-1,2,3,4-tetrahydroquinazolin-6-yl)urea

A sample of 1-(tert-butyl)-3-(3-methyl-2-oxo-1-(1-phenylethyl)-1,2,3,4-tetrahydroquinazolin-6-yl)urea (170 mg) was separated by chiral-HPLC using a (R, R)-WHELK-O1-Kromasil 2.11*25 cm, 5 μm column (eluent: MtBE(0.5% 2M NH₃-MeOH)—HPLC and Hex(EtOH—HPLC) to yield first example cyclopentyl 3-tert-butyl-1-{3-methyl-2-oxo-1-[(1R)-1-phenylethyl]-4H-quinazolin-6-yl}urea (26.6 mg). LC/MS (ESI): mass calcd. for C₂₂H₂₈N₄O₂:380.22 m/z, found:381.05 [M+H]⁺. ¹H NMR (400 MHz, DMSO-d6) δ 8.05 (s, 1H), 7.26-7.37 (m, 5H), 6.80 (m, 1H), 6.37 (m, 1H), 5.92 (m, 2H), 4.32 (m, 2H), 2.92 (s, 3H), 1.74 (m, 3H), 1.19-1.25 (s, 10H). And to yield second enantiomer example cyclopentyl 3-tert-butyl-1-{3-methyl-2-oxo-1-[(1S)-1-phenylethyl]-4H-quinazolin-6-yl}urea (41.6 mg). LC/MS (ESI): mass calcd. for C₂₂H₂₈N₄O₂:380.22 m/z, found:381.10 [M+H]⁺. ¹H NMR (400 MHz, DMSO-d₆) δ 8.05 (s, 1H), 7.26-7.37 (m, 5H), 6.80 (m, 1H), 6.37 (m, 1H), 5.92 (m, 2H), 4.32 (m, 2H), 2.92 (s, 3H), 1.74 (m, 3H), 1.19-1.25 (m, 10H).

### Example 46 and Example 52: (S)-1-(tert-butyl)-3-(1-(1-(4-chlorophenyl)ethyl)-3-methyl-2-oxo-1,2,3,4-tetrahydroquinazolin-6-yl)urea and (R)-1-(tert-butyl)-3-(1-(1-(4-chlorophenyl)ethyl)-3-methyl-2-oxo-1,2,3,4-tetrahydroquinazolin-6-yl)urea

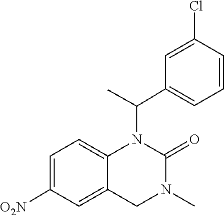 [SMILES: CC(c1cccc(Cl)c1)N1C(=O)N(C)Cc2cc([N+](=O)[O-])ccc21]

### 1-(1-(3-chlorophenyl)ethyl)-3-methyl-6-nitro-3,4-dihydroquinazolin-2(1H)-one

To a stirred mixture of 3-methyl-6-nitro-1,4-dihydroquinazolin-2-one (500 mg, 2.413 mmol, 1 equiv) and Cs₂CO₃ (1.57 g, 4.826 mmol, 2 equiv) in DMF (10 mL) was added 1-(1-bromoethyl)-3-chlorobenzene (635.68 mg, 2.896 mmol, 1.2 equiv) in portions at 25° C. under air atmosphere. The resulting mixture was stirred for 2 h at room temperature. Desired product could be detected by LCMS. The mixture was diluted with water (50 mL). The resulting mixture was extracted with EA (3×50 mL). The combined organic layers were dried over anhydrous Na₂SO₄. After filtration, the filtrate was concentrated under reduced pressure. The residue was purified by silica gel column chromatography, eluted with PE/EA (1:1) to afford 1-(1-(3-chlorophenyl)ethyl)-3-methyl-6-nitro-3,4-dihydroquinazolin-2(1H)-one (640 mg, 76.70%) as a yellow solid. MS (ESI): mass calcd. for C₁₇H₁₆ClN₃O₃: 345.09 m/z, found 345.78 [M+H]⁺.

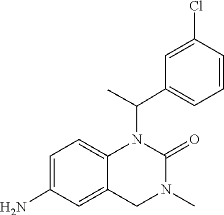 [SMILES: CC(c1cccc(Cl)c1)N1C(=O)N(C)Cc2cc(N)ccc21]

### 6-amino-1-(1-(3-chlorophenyl)ethyl)-3-methyl-3,4-dihydroquinazolin-2(1H)-one

To a stirred mixture of 1-(1-(3-chlorophenyl)ethyl)-3-methyl-6-nitro-3,4-dihydroquinazolin-2(1H)-one (630 mg, 1.822 mmol, 1 equiv) and Fe (1017.48 mg, 18.220 mmol, 10 equiv) in EtOH (8 mL) and H₂O (0.8 mL) was added NH₄Cl (974.57 mg, 18.220 mmol, 10 equiv) in portions at 70 degrees C. under air atmosphere. The resulting mixture was stirred for 2 h at room temperature. Desired product could be detected by LCMS. The mixture was diluted with water (100 mL). The resulting mixture was extracted with EA (3×100 mL). The combined organic layers were dried over anhydrous Na₂SO₄. After filtration, the filtrate was concentrated under reduced pressure. The residue was purified by silica gel column chromatography, eluted with PE/EA (1:1) to afford 6-amino-1-(1-(3-chlorophenyl)ethyl)-3-methyl-3,4-dihydroquinazolin-2(1H)-one (240 mg, 41.71%) as a yellow solid. MS (ESI): mass calcd. for C₁₆H₁₆ClN₃O: 315.11 m/z, found 315.80 [M+H]⁺.

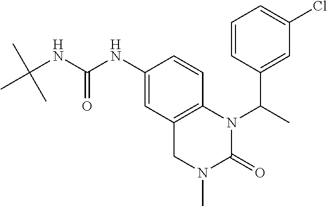 [SMILES: CC(c1cccc(Cl)c1)N1C(=O)N(C)Cc2cc(NC(=O)NC(C)(C)C)ccc21]

### 1-(tert-butyl)-3-(1-(1-(3-chlorophenyl)ethyl)-3-methyl-2-oxo-1,2,3,4-tetrahydroquinazolin-6-yl)urea

To a stirred mixture of 6-amino-1-(1-(3-chlorophenyl)ethyl)-3-methyl-3,4-dihydroquinazolin-2(1H)-one(242 mg, 0.766 mmol, 1 equiv) and 2-isocyanato-2-methylpropane (151.93 mg, 1.532 mmol, 2 equiv) in DCM (5 mL) was added TEA (232.63 mg, 2.298 mmol, 3 equiv) at 25 degrees C. under air atmosphere. The resulting mixture was stirred for 2 h at room temperature. Desired product could be detected by LCMS. The mixture was diluted with water (30 mL). The resulting mixture was extracted with EA (3×30 mL). The organic layers were combined, dried over anhydrous sodium sulfate, filtered, and concentrated. The residue was purified by silica gel column chromatography, eluted with PE/EA (1:1) to afford 1-(tert-butyl)-3-(1-(1-(3-chlorophenyl)ethyl)-3-methyl-2-oxo-1,2,3,4-tetrahydroquinazolin-6-yl)urea (110 mg, 34.60%) as a white solid. MS (ESI): mass calcd. for C₂₂H₂₇ClN₄O₂: 414.18 m/z, found 414.93[M+H]⁺

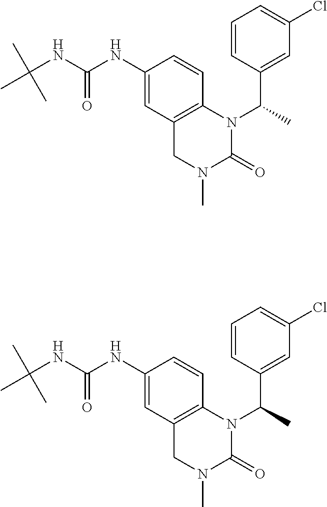 [SMILES: C[C@@H](c1cccc(Cl)c1)N1C(=O)N(C)Cc2cc(NC(=O)NC(C)(C)C)ccc21 ; C[C@H](c1cccc(Cl)c1)N1C(=O)N(C)Cc2cc(NC(=O)NC(C)(C)C)ccc21]

### (S)-1-(tert-butyl)-3-(1-(1-(4-chlorophenyl)ethyl)-3-methyl-2-oxo-1,2,3,4-tetrahydroquinazolin-6-yl)urea and (R)-1-(tert-butyl)-3-(1-(1-(4-chlorophenyl)ethyl)-3-methyl-2-oxo-1,2,3,4-tetrahydroquinazolin-6-yl)urea

The 1-(tert-butyl)-3-(1-(1-(4-chlorophenyl)ethyl)-3-methyl-2-oxo-1,2,3,4-tetrahydroquinazolin-6-yl)urea (110 mg) was separated by chiral-HPLC using a CHIRALPAK ID, 2*25 cm, 5 μm column (eluent: 10% to 10% (v/v): EtOH—HPLC and Hex(0.5% 2M NH₃-MeOH) to yield first enantiomer example (R)-1-(tert-butyl)-3-(1-(1-(4-chlorophenyl)ethyl)-3-methyl-2-oxo-1,2,3,4-tetrahydroquinazolin-6-yl)urea (24.4 mg, 7.57%) as a white solid. LC/MS: mass calcd for C₂₂H₂₇ClN₄O₂: 414.18, found: 415.00 [M+H]⁺. ¹H-NMR (300 MHz, DMSO-d6) δ 8.07 (s, 1H), 7.32-7.39 (m, 1H), 7.20-7.30 (m, 4H), 6.80-6.92 (m, 1H), 6.39 (d, J=8.8 Hz, 1H), 5.75-5.96 (m, 2H), 4.32 (s, 2H), 2.92 (s, 3H), 1.74 (d, J=7.1 Hz, 3H), 1.26 (s, 9H). And to yield second enantiomer example (S)-1-(tert-butyl)-3-(1-(1-(4-chlorophenyl)ethyl)-3-methyl-2-oxo-1,2,3,4-tetrahydroquinazolin-6-yl)urea (16.4 mg, 5.19%) as a white solid. LC/MS: mass calcd. for C₂₂H₂₇ClN₄O₂: 414.18, found: 415.00 [M+H]⁺. ¹H-NMR (300 MHz, DMSO-d6) δ 8.07 (s, 1H), 7.20-7.42 (m, 5H), 6.81-6.91 (m, 1H), 6.38 (d, J=8.8 Hz, 1H), 5.78-5.96 (m, 2H), 4.32 (s, 2H), 2.91 (s, 3H), 1.73 (d, J=7.1 Hz, 3H), 1.25 (s, 9H).

### Example 47: 1-(tert-butyl)-3-(1-(3-chlorobenzyl)-3-methyl-2-oxo-1,2,3,4-tetrahydroquinazolin-6-yl)urea

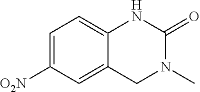

### 3-methyl-6-nitro-3,4-dihydroquinazolin-2(1H)-one

To a stirred mixture of 3-methyl-1,4-dihydroquinazolin-2-one (1 g, 6.166 mmol, 1 equiv) in H₂SO₄ (30 mL) was added KNO₃ (0.50 g, 4.933 mmol, 0.8 equiv) in portions at 0° C. under air atmosphere. The resulting mixture was stirred for 2 h at room temperature. Desired product could be detected by LCMS. The mixture was diluted with water (100 mL). The resulting mixture was extracted with EA (3×100 mL). The combined organic layers were dried over anhydrous Na₂SO₄. After filtration, the filtrate was concentrated under reduced pressure. The residue was purified by silica gel column chromatography, eluted with PE/EA (1:1) to afford 3-methyl-6-nitro-3,4-dihydroquinazolin-2(1H)-one (400 mg, 31.31%) as a white solid. MS (ESI): mass calcd. for C₉H₉N₃O₃: 207.06 m/z, found 208.19 [M+H]⁺.

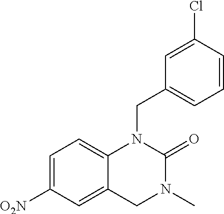 [SMILES: CN1Cc2cc([N+](=O)[O-])ccc2N(Cc2cccc(Cl)c2)C1=O]

### 1-(3-chlorobenzyl)-3-methyl-6-nitro-3,4-dihydroquinazolin-2(1H)-one

To a stirred mixture of 3-methyl-6-nitro-3,4-dihydroquinazolin-2(1H)-one (300 mg, 1.448 mmol, 1 equiv) and 1-(bromomethyl)-3-chlorobenzene (357.03 mg, 1.738 mmol, 1.2 equiv) in DMF (5 mL) was added Cs₂CO₃ (1419.69 mg, 4.344 mmol, 3 equiv) in portions at 25 degrees C. under air atmosphere. The resulting mixture was stirred for 2 h at room temperature. Desired product could be detected by LCMS. The mixture was diluted with water (50 mL). The resulting mixture was extracted with EA (3×50 mL). The combined organic layers were dried over anhydrous Na₂SO₄. After filtration, the filtrate was concentrated under reduced pressure. The residue was purified by silica gel column chromatography, eluted with PE/EA (1:1) to afford 1-(3-chlorobenzyl)-3-methyl-6-nitro-3,4-dihydroquinazolin-2(1H)-one (400 mg, 83.27%) as a yellow solid. MS (ESI): mass calcd. for C₁₆H₁₄ClN₃O₃: 331.07 m/z, found 331.76 [M+H]⁺.

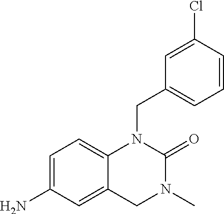 [SMILES: CN1Cc2cc(N)ccc2N(Cc2cccc(Cl)c2)C1=O]

### 6-amino-1-(3-chlorobenzyl)-3-methyl-3,4-dihydroquinazolin-2(1H)-one

To a stirred mixture of 1-(3-chlorobenzyl)-3-methyl-6-nitro-3,4-dihydroquinazolin-2(1H)-one (395 mg, 1.191 mmol, 1 equiv) and Fe (664.90 mg, 11.910 mmol, 10 equiv) in EtOH (5 mL) and H₂O (0.5 mL) was added NH₄Cl (636.86 mg, 11.910 mmol, 10 equiv) in portions at 25 degrees C. under air atmosphere. The resulting mixture was stirred for 2 h at 60 degrees C. Desired product could be detected by LCMS. The mixture was diluted with water (50 mL). The resulting mixture was extracted with EA (3×50 mL). The organic layers were combined, dried over anhydrous sodium sulfate, filtered, and concentrated. The residue was purified by silica gel column chromatography, eluted with PE/EA (1:1) to afford 6-amino-1-(3-chlorobenzyl)-3-methyl-3,4-dihydroquinazolin-2(1H)-one (160 mg, 44.53%) as a yellow solid. MS (ESI): mass calcd. for C₁₆H₁₆ClN₃O: 301.10 m/z, found 301.77[M+H]⁺.

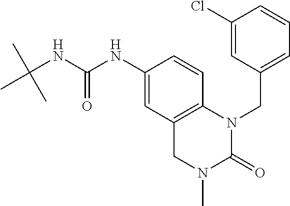 [SMILES: CN1Cc2cc(NC(=O)NC(C)(C)C)ccc2N(Cc2cccc(Cl)c2)C1=O]

### 1-(tert-butyl)-3-(1-(3-chlorobenzyl)-3-methyl-2-oxo-1,2,3,4-tetrahydroquinazolin-6-yl)urea

To a stirred mixture of 6-amino-1-(3-chlorobenzyl)-3-methyl-3,4-dihydroquinazolin-2(1H)-one (150 mg, 0.497 mmol, 1 equiv) and 2-isocyanato-2-methylpropane (98.55 mg, 0.994 mmol, 2 equiv) in dichloromethane (1 mL) was added TEA (150.90 mg, 1.491 mmol, 3 equiv) dropwise at 0 degrees C. under air atmosphere. The resulting mixture was stirred for 2 h at room temperature. Desired product could be detected by LCMS. The mixture was diluted with water (30 mL). The resulting mixture was extracted with EA (3×30 mL). The organic layers were combined, dried over anhydrous sodium sulfate, filtered, and concentrated. The residue was purified by silica gel column chromatography, eluted with PE/EA (1:1) to afford the 1-(tert-butyl)-3-(1-(3-chlorobenzyl)-3-methyl-2-oxo-1,2,3,4-tetrahydroquinazolin-6-yl)urea(31.9 mg, 15.65%) as a white solid. MS (ESI): mass calcd. for C₂₁H₂₅ClN₄O₂: 400.17 m/z, found 401.00[M+H]⁺. ¹H-NMR (300 MHz, DMSO-d6) δ 8.11 (s, 1H), 7.22-7.40 (m, 4H), 7.18 (d, J=7.4 Hz, 1H), 6.90-6.99 (m, 1H), 6.56 (d, J=8.7 Hz, 1H), 5.91 (s, 1H), 5.02 (s, 2H), 4.42 (s, 2H), 2.95 (s, 3H), 1.26 (s, 9H).

### Example 48: 1-(tert-butyl)-3-(1-(2-fluoro-3-methylbenzyl)-2-oxo-1,2,3,4-tetrahydroquinolin-6-yl)urea

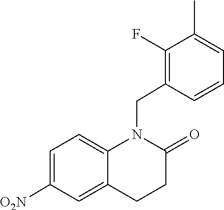 [SMILES: Cc1cccc(CN2C(=O)CCc3cc([N+](=O)[O-])ccc32)c1F]

### 1-(2-fluoro-3-methylbenzyl)-6-nitro-3,4-dihydroquinolin-2(1H)-one

To a stirred solution of 6-nitro-3,4-dihydro-1H-quinolin-2-one (300 mg, 1.561 mmol, 1 equiv) in DMF (5 mL) was added 1-(bromomethyl)-2-fluoro-3-methylbenzene (412.08 mg, 2.029 mmol, 1.3 equiv) and Cs₂CO₃ (1.53 g, 4.683 mmol, 3 equiv) at rt and stirred for overnight. After completion of reaction, the resulting mixture was extracted with EA (3×30 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (3:1) to afford 1-(2-fluoro-3-methylbenzyl)-6-nitro-3,4-dihydroquinolin-2(1H)-one (420 mg, 85.60%) as a yellow solid. MS (ESI): mass calcd. for C₁₇H₁₅FN₂O₃: 314.11 m/z, found 315.05 [M+H]⁺.

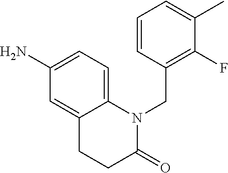 [SMILES: Cc1cccc(CN2C(=O)CCc3cc(N)ccc32)c1F]

### 6-amino-1-(2-fluoro-3-methylbenzyl)-3,4-dihydroquinolin-2(1H)-one

To a solution of 1-(2-fluoro-3-methylbenzyl)-6-nitro-3,4-dihydroquinolin-2(1H)-one (200 mg, 0.636 mmol, 1 equiv) in 20 mL MeOH was added Pd/C (30 mg) under nitrogen atmosphere. The mixture was hydrogenated at room temperature for 50 min under hydrogen atmosphere using a hydrogen balloon, filtered through a Celite pad and concentrated under reduced pressure to afford 6-amino-1-(2-fluoro-3-methylbenzyl)-3,4-dihydroquinolin-2(1H)-one (160 mg, 88.44%) as a white solid. MS (ESI): mass calcd. for C₁₇H₁₇FN₂O: 284.13 m/z, found 285.10 [M+H]⁺.

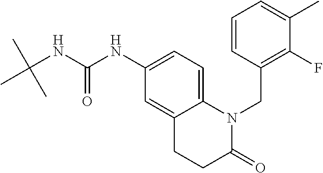 [SMILES: Cc1cccc(CN2C(=O)CCc3cc(NC(=O)NC(C)(C)C)ccc32)c1F]

### 1-(tert-butyl)-3-(1-(2-fluoro-3-methylbenzyl)-2-oxo-1,2,3,4-tetrahydroquinolin-6-yl)urea

To a stirred solution of 6-amino-1-[(2-fluoro-3-methylphenyl)methyl]-3,4-dihydroquinolin-2-one (80 mg, 0.281 mmol, 1 equiv) in DCM (5 mL) was added 2-isocyanato-2-methylpropane (139.46 mg, 1.405 mmol, 5 equiv) and Et₃N (85.41 mg, 0.843 mmol, 3 equiv) at rt and stirred for overnight. The reaction progress was monitored by LCMS. After completion of reaction, the reaction mixture was concentrated under reduced pressure to give crude product which was further purified by Prep-HPLC with the following conditions Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0 to afford 3-tert-butyl-1-{1-[(2-fluoro-3-methylphenyl)methyl]-2-oxo-3,4-dihydroquinolin-6-yl}urea (29.0 mg, 26.78%) as a white solid. MS (ESI): mass calcd. for C₂₂H₂₆FN₃O₂: 383.20 m/z, found 384.20 [M+H]⁺. ¹H NMR (300 MHz, DMSO-d₆) δ 8.13 (s, 1H), 7.34 (d, J=2.4 Hz, 1H), 7.15 (t, J=7.3 Hz, 1H), 6.92-7.04 (m, 2H), 6.76-6.88 (m, 1H), 6.71 (d, J=8.7 Hz, 1H), 5.93 (s, 1H), 5.09 (s, 2H), 2.89 (dd, J=8.7, 5.8 Hz, 2H), 2.65 (dd, J=8.7, 5.8 Hz, 2H), 2.25 (d, J=2.0 Hz, 3H), 1.27 (s, 9H).

### Example 49: 1-(tert-butyl)-3-(1-(2-fluoro-5-methylbenzyl)-2-oxo-1,2,3,4-tetrahydroquinolin-6-yl)urea

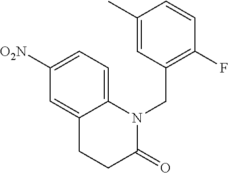 [SMILES: Cc1ccc(F)c(CN2C(=O)CCc3cc([N+](=O)[O-])ccc32)c1]

### 1-(2-fluoro-5-methylbenzyl)-6-nitro-3,4-dihydroquinolin-2(1H)-one

To a stirred solution of 6-nitro-3,4-dihydro-1H-quinolin-2-one (300 mg, 1.561 mmol, 1 equiv) in DMF (5 mL) was added 2-(bromomethyl)-1-fluoro-4-methylbenzene (412.08 mg, 2.029 mmol, 1.3 equiv) and Cs₂CO₃ (1.53 g, 4.683 mmol, 3 equiv) at rt and stirred for overnight. After completion of reaction, the resulting mixture was extracted with EA (3×20 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (3:1) to afford 1-(2-fluoro-5-methylbenzyl)-6-nitro-3,4-dihydroquinolin-2(1H)-one (400 mg, 81.52%) as a yellow solid. MS (ESI): mass calcd. for C₁₇H₁₅FN₂O: 314.11 m/z, found 315.05 [M+H]+.

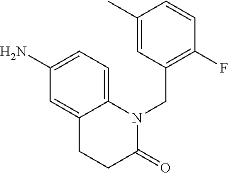 [SMILES: Cc1ccc(F)c(CN2C(=O)CCc3cc(N)ccc32)c1]

### 6-amino-1-(2-fluoro-5-methylbenzyl)-3,4-dihydroquinolin-2(1H)-one

To a solution of 1-(2-fluoro-5-methylbenzyl)-6-nitro-3,4-dihydroquinolin-2(1H)-one (200 mg, 0.636 mmol, 1 equiv) in 20 mL MeOH was added Pd/C (30 mg) under nitrogen atmosphere. The mixture was hydrogenated at room temperature for 30 min under hydrogen atmosphere using a hydrogen balloon, filtered through a Celite pad and concentrated under reduced pressure to afford 6-amino-1-(2-fluoro-5-methylbenzyl)-3,4-dihydroquinolin-2(1H)-one (160 mg, 88.40%) as a white solid. MS (ESI): mass calcd. for C₁₇H₁₇FN₂O: 284.13 m/z, found 285.10 [M+H]+.

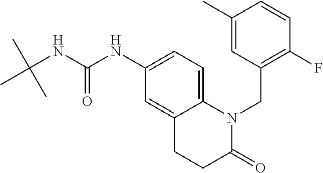 [SMILES: Cc1ccc(F)c(CN2C(=O)CCc3cc(NC(=O)NC(C)(C)C)ccc32)c1]

### 1-(tert-butyl)-3-(1-(2-fluoro-5-methylbenzyl)-2-oxo-1,2,3,4-tetrahydroquinolin-6-yl)urea

To a stirred solution of 6-amino-1-[(2-fluoro-5-methylphenyl)methyl]-3,4-dihydroquinolin-2-one (80 mg, 0.281 mmol, 1 equiv) in DCM (5 mL) was added 2-isocyanato-2-methylpropane (139.46 mg, 1.405 mmol, 5 equiv) and Et₃N (85.41 mg, 0.843 mmol, 3 equiv) at rt and stirred for overnight. The reaction progress was monitored by LCMS. After completion of reaction, the reaction mixture was concentrated under reduced pressure to give crude product which was further purified by HPLC with the following conditions Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0 to afford 1-(tert-butyl)-3-(1-(2-fluoro-5-methylbenzyl)-2-oxo-1,2,3,4-tetrahydroquinolin-6-yl)urea (8.1 mg, 7.49%) as a white solid. MS (ESI): mass calcd. for C₂₂H₂₆FN₃O₂: 383.20 m/z, found 384.20 [M+H]⁺. ¹H NMR (300 MHz, DMSO-d₆) δ 8.13 (s, 1H), 7.34 (d, J=2.4 Hz, 1H), 7.16-7.04 (m, 2H), 7.00 (dd, J=8.7, 2.5 Hz, 1H), 6.81 (d, J=7.5 Hz, 1H), 6.71 (d, J=8.8 Hz, 1H), 5.93 (s, 1H), 5.06 (s, 2H), 2.89 (t, J=7.2 Hz, 2H), 2.66 (dd, J=8.8, 5.8 Hz, 2H), 2.19 (s, 3H), 1.27 (s, 9H).

### Example 50: 1-(tert-butyl)-3-(3-oxo-4-(3-(trifluoromethyl)benzyl)-3,4-dihydro-2H-benzo[b][1,4]oxazin-7-yl)urea

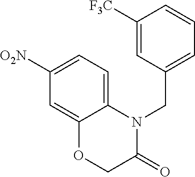 [SMILES: O=C1COc2cc([N+](=O)[O-])ccc2N1Cc1cccc(C(F)(F)F)c1]

### 7-nitro-4-(3-(trifluoromethyl)benzyl)-2H-benzo[b][1,4]oxazin-3(4H)-one

To a solution of 7-nitro-2H-benzo[b][1,4]oxazin-3(4H)-one (500 mg, 2.575 mmol, 1 equiv) in DMF (10 mL) was added Cs₂CO₃ (1.678 g, 5.150 mmol, 2 equiv) and 1-(bromomethyl)-3-(trifluoromethyl)benzene (676.8 mg, 2.832 mmol, 1.1 equiv). The resulting mixture was stirred for 16 h at rt. The mixture was diluted with EtOAc (100 mL), washed with water (3×30 mL), brine (2×30 mL) and concentrated under vacuum to yield a crude product which was directly purified by silica gel column, eluted with PE/EA (3:1) to afford 7-nitro-4-(3-(trifluoromethyl)benzyl)-2H-benzo[b][1,4]oxazin-3(4H)-one (473.8 mg, 52.23%) as a white solid. MS (ESI): mass calcd. for C₁₆H₁₁F₃N₂O₄: 352.07 m/z, found 353.05 [M+H]⁺.

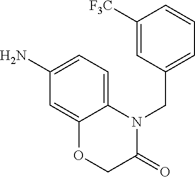 [SMILES: Nc1ccc2c(c1)OCC(=O)N2Cc1cccc(C(F)(F)F)c1]

### 7-amino-4-(3-(trifluoromethyl)benzyl)-2H-benzo[b][1,4]oxazin-3(4H)-one

To a solution of 7-nitro-4-(3-(trifluoromethyl)benzyl)-2H-benzo[b][1,4]oxazin-3(4H)-one (300 mg, 0.852 mmol, 1 equiv) in MeOH (10 mL) was added Pd/C (100 mg). The reaction mixture was then stirred for 1 h under hydrogen atmosphere (balloon). The reaction mixture was filtered through a pad of Celite and concentrated under reduced pressure to afford 7-amino-4-(3-(trifluoromethyl)benzyl)-2H-benzo[b][1,4]oxazin-3(4H)-one (263.6 mg, 96.0%) as a white solid. MS (ESI): mass calcd. for C₁₆H₁₃F₃N₂O₂, 322.09 m/z, found 323.05 [M+H]⁺.

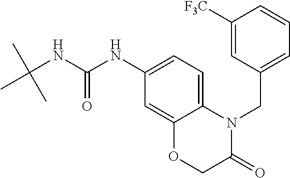 [SMILES: CC(C)(C)NC(=O)Nc1ccc2c(c1)OCC(=O)N2Cc1cccc(C(F)(F)F)c1]

### 1-(tert-butyl)-3-(3-oxo-4-(3-(trifluoromethyl)benzyl)-3,4-dihydro-2H-benzo[b][1,4]oxazin-7-yl)urea

To a stirred solution of 7-amino-4-(3-(trifluoromethyl)benzyl)-2H-benzo[b][1,4]oxazin-3(4H)-one (90 mg, 0.279 mmol, 1 equiv) in DCM (6 mL) & toluene (2 mL) was added 2-isocyanato-2-methylpropane (82.97 mg, 0.837 mmol, 3 equiv) stirred 16 h at 90° C. The reaction mixture was concentrated under vacuum to yield a crude product which was directly purified Prep-HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 9; Number Of Runs: 0) to afford 1-(tert-butyl)-3-(3-oxo-4-(3-(trifluoromethyl)benzyl)-3,4-dihydro-2H-benzo[b][1,4]oxazin-7-yl)urea (2.1 mg, 1.7%) as a white solid. MS (ESI): mass calcd. for C₂₁H₂₂F₃N₃O₃, 421.16 m/z, found 422.15 [M+H]⁺. ¹H NMR (300 MHz, DMSO-d₆) δ 8.22 (s, 1H), 7.49-7.70 (m, 4H), 7.21 (d, J=2.3 Hz, 1H), 6.87-6.90 (m, 1H), 6.75-6.79 (m, 1H), 5.94 (s, 1H), 5.19-5.23 (m, 2H), 4.75-4.78 (m, 2H), 1.23-1.29 (m, 9H).

### Example 51: 1-(1-benzyl-3-(hydroxymethyl)-2-oxo-1,2,3,4-tetrahydroquinolin-6-yl)-3-(tert-butyl)urea

### Synthetic Scheme

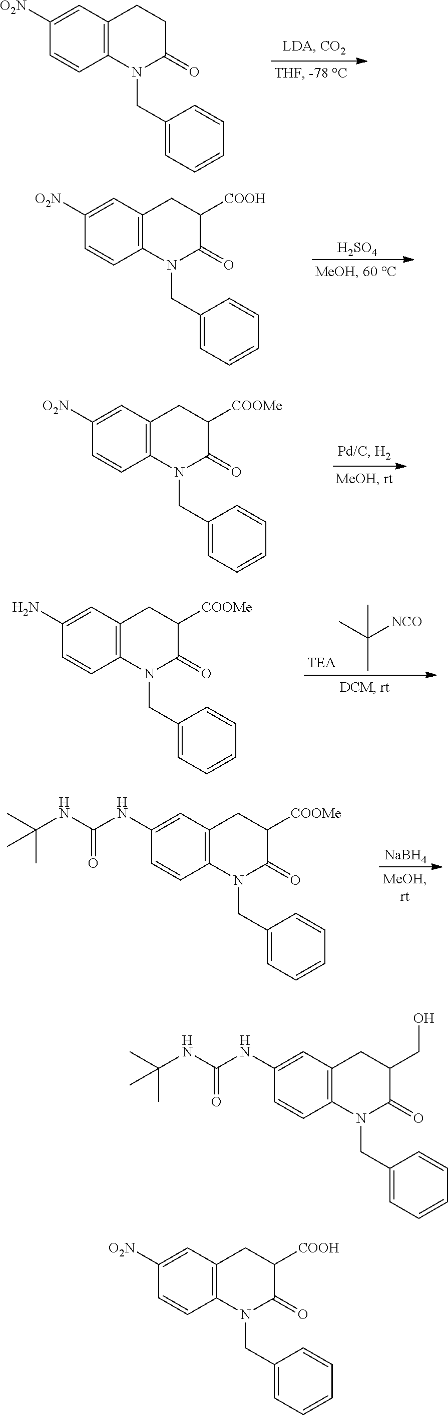

### 1-benzyl-6-nitro-2-oxo-1,2,3,4-tetrahydroquinoline-3-carboxylic acid

In a 50-mL round bottom flask, to a solution of 1-benzyl-6-nitro-3,4-dihydroquinolin-2-one (500 mg, 1.771 mmol, 1 equiv) in tetrahydrofuran (5 mL, 0.069 mmol, 0.04 equiv) was added dropwise 2M LDA (in THF) (1 mL, 1.948 mmol, 1.1 equiv) at −78° C. under N₂. The reaction mixture was stirred at −78° C. for 30 min. The reaction was quenched with dry ice. The mixture was stirred for 10 min at rt. NH₄Cl (aq.) was added into the mixture. The mixture was acidified pH=5 with HCl (1N). The solution was extracted with EA (3×50 mL). The combined organic extracts were washed with brine (10 mL), dried over anhydrous Na₂SO₄, and concentrated under vacuum. The residue was purified by silica gel column, eluted with DCM/MeOH (8:1) to afford 1-benzyl-6-nitro-2-oxo-1,2,3,4-tetrahydroquinoline-3-carboxylic acid (150 mg, 25.95%) as an off-white solid. MS (ESI): mass calcd. for C₁₇H₁₄N₂O₅, 326.09 m/z, found 327.05 [M+H]⁺.

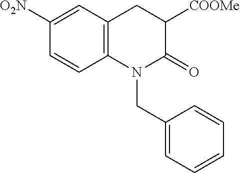 [SMILES: COC(=O)C1Cc2cc([N+](=O)[O-])ccc2N(Cc2ccccc2)C1=O]

### methyl 1-benzyl-6-nitro-2-oxo-1,2,3,4-tetrahydroquinoline-3-carboxylate

A solution of 1-benzyl-6-nitro-2-oxo-1,2,3,4-tetrahydroquinoline-3-carboxylic acid (500 mg, 1.532 mmol, 1 equiv) in methanol (10 mL) was added sulfuric acid (0.1 mL) at rt. The resulting mixture was stirred for 2 h at 70° C. Quench with water (10 mL) and extract with ethyl acetate (3×30 mL). The combined organic layers were washed with brine, dried over anhydrous Na₂SO₄. After filtration, the filtrate was concentrated under reduced pressure. The residue was purified by silica gel column, eluted with PE/EA (3:1) to afford methyl 1-benzyl-6-nitro-2-oxo-1,2,3,4-tetrahydroquinoline-3-carboxylate (150 mg, 28.76%) as white solid. MS (ESI): mass calcd. for C₁₈H₁₆N₂O₅, 340.11 m/z, found 341.05 [M+H]⁺.

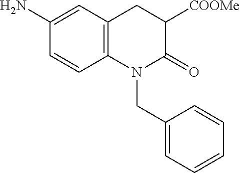 [SMILES: COC(=O)C1Cc2cc(N)ccc2N(Cc2ccccc2)C1=O]

### methyl 6-amino-1-benzyl-2-oxo-1,2,3,4-tetrahydroquinoline-3-carboxylate

To a solution of methyl 1-benzyl-6-nitro-2-oxo-1,2,3,4-tetrahydroquinoline-3-carboxylate (200 mg, 0.588 mmol, 1 equiv) in MeOH (10 mL) was added Pd/C (30 mg). The reaction mixture was then stirred for 1 h under hydrogen atmosphere (balloon). The reaction mixture was filtered through a pad of Celite and concentrated under reduced pressure to afford methyl 6-amino-1-benzyl-2-oxo-1,2,3,4-tetrahydroquinoline-3-carboxylate (150 mg, 79.36%) as a yellow solid. MS (ESI): mass calcd. for C₁₈H₁₈N₂O₃, 310.13 m/z, found 311.15 [M+H]⁺.

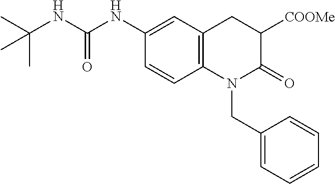 [SMILES: COC(=O)C1Cc2cc(NC(=O)NC(C)(C)C)ccc2N(Cc2ccccc2)C1=O]

### methyl 1-benzyl-6-(3-(tert-butyl)ureido)-2-oxo-1,2,3,4-tetrahydroquinoline-3-carboxylate

To a stirred solution of methyl 6-amino-1-benzyl-2-oxo-1,2,3,4-tetrahydroquinoline-3-carboxylate (150 mg, 0.483 mmol, 1 equiv) in DCM (8 mL) was added 2-isocyanato-2-methylpropane (239.5 mg, 2.417 mmol, 5 equiv) and TEA (146.7 mg, 1.450 mmol, 3 equiv) stirred overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) to afford methyl 1-benzyl-6-[(tert-butylcarbamoyl)amino]-2-oxo-3,4-dihydroquinoline-3-carboxylate (70 mg, 31.95%) as a yellow solid. MS (ESI): mass calcd. for C₂₃H₂₇N₃O₄, 409.20 m/z, found 410.25 [M+H]⁺.

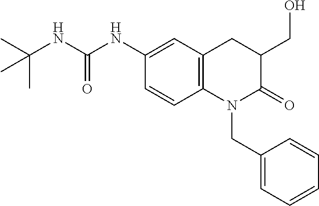 [SMILES: CC(C)(C)NC(=O)Nc1ccc2c(c1)CC(CO)C(=O)N2Cc1ccccc1]

### 1-(1-benzyl-3-(hydroxymethyl)-2-oxo-1,2,3,4-tetrahydroquinolin-6-yl)-3-(tert-butyl)urea

To a stirred solution of methyl 1-benzyl-6-[(tert-butylcarbamoyl)amino]-2-oxo-3,4-dihydroquinoline-3-carboxylate (70 mg, 0.171 mmol, 1 equiv) in MeOH (5 mL) was added NaBH₄ (64.6 mg, 1.709 mmol, 10 equiv) stirred 3 h at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 1-[1-benzyl-3-(hydroxymethyl)-2-oxo-3,4-dihydroquinolin-6-yl]-3-tert-butylurea (9.9 mg, 15.13%) as a white solid. MS (ESI): mass calcd. for C₂₂H₂₇N₃O₃, 381.21 m/z, found 382.20 [M+H]⁺. ¹H NMR (300 MHz, DMSO-d₆) δ 8.11 (s, 1H), 7.25-7.36 (m, 3H), 7.12-7.24 (m, 3H), 6.96 (dd, J=8.6, 2.4 Hz, 1H), 6.74 (d, J=8.8 Hz, 1H), 5.93 (s, 1H), 5.08 (s, 2H), 4.78 (s, 1H), 3.68-3.83 (m, 1H), 3.59 (d, J=10.8 Hz, 1H), 2.98 (dd, J=15.4, 5.6 Hz, 1H), 2.75-2.90 (m, 1H), 2.65-2.75 (m, 1H), 1.26 (s, 9H).

### Example 53: 3-tert-butyl-1-{1-[(2-cyano-3-methylphenyl)methyl]-2-oxo-3,4-dihydroquinolin-6-yl}urea

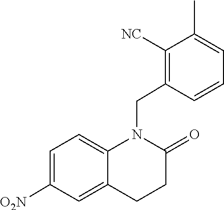 [SMILES: Cc1cccc(CN2C(=O)CCc3cc([N+](=O)[O-])ccc32)c1C#N]

### 2-methyl-6-[(6-nitro-2-oxo-3,4-dihydroquinolin-1-yl)methyl]benzonitrile

To a stirred solution of 2-(bromomethyl)-6-methylbenzonitrile (400 mg, 1.904 mmol, 1 equiv) and 6-nitro-3,4-dihydro-1H-quinolin-2-one (548.88 mg, 2.856 mmol, 1.5 equiv) in DMF (15 mL) was added K₂CO₃ (662.69 mg, 4.760 mmol, 2.5 equiv) in portions at room temperature under nitrogen atmosphere. The final reaction mixture was stirred for 2 h at room temperature. Quenched with water (20 mL) and extracted with EA (3×20 mL). The combined extracts were washed with water, brine, dried over Na₂SO₄, filtered, and concentrated. The crude product was purified by silica gel column chromatography, eluted with PE/EA (1:1) to afford 2-methyl-6-[(6-nitro-2-oxo-3,4-dihydroquinolin-1-yl)methyl]benzonitrile (400 mg, 65.38%) as a white solid. MS: mass calcd. for C₁₈H₁₅N₃O₃: 321.11 m/z, found 322.05 [M+H]⁺.

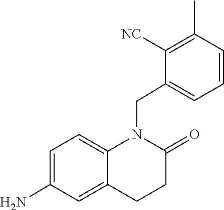 [SMILES: Cc1cccc(CN2C(=O)CCc3cc(N)ccc32)c1C#N]

### 2-[(6-amino-2-oxo-3,4-dihydroquinolin-1-yl)methyl]-6-methylbenzonitrile

To a stirred solution of 2-methyl-6-[(6-nitro-2-oxo-3,4-dihydroquinolin-1-yl)methyl]benzonitrile (400 mg, 1.245 mmol, 1 equiv) in EtOH/H₂O (11 mL, 10/1) was added Fe (695.16 mg, 12.450 mmol, 10 equiv) and NH₄Cl (665.85 mg, 12.450 mmol, 10 equiv) in portions at room temperature under nitrogen atmosphere. The final reaction mixture was stirred for 2 h at 60° C. The reaction was quenched with water at rt. After filtration, the filter cake was washed with EA (3×20 mL). The filtrate was added water (20 mL) and extracted with EA (3×20 mL). The combined extracts were washed with water, brine, dried over Na₂SO₄, filtered, and concentrated. The resulting mixture was concentrated under vacuum. The crude product was purified by silica gel column chromatography, eluted with PE/EA (1:1) to afford 2-[(6-amino-2-oxo-3,4-dihydroquinolin-1-yl)methyl]-6-methylbenzonitrile (300 mg, 82.72%) as a light yellow solid. MS: mass calcd. for C₁₈H₁₇N₃O: 291.14 m/z, found 292.10 [M+H]⁺.

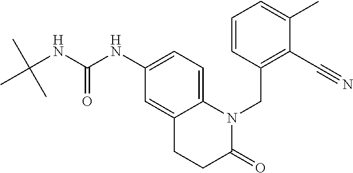 [SMILES: Cc1cccc(CN2C(=O)CCc3cc(NC(=O)NC(C)(C)C)ccc32)c1C#N]

### 3-tert-butyl-1-{1-[(2-cyano-3-methylphenyl)methyl]-2-oxo-3,4-dihydroquinolin-6-yl}urea

To a stirred solution of 2-[(6-amino-2-oxo-3,4-dihydroquinolin-1-yl)methyl]-6-methylbenzonitrile (150 mg, 0.515 mmol, 1 equiv) in toluene/DCM (10 mL, ½) was added 2-isocyanato-2-methylpropane (153.11 mg, 1.545 mmol, 3 equiv) in portions at room temperature under nitrogen atmosphere. The final reaction mixture was stirred for 3 h at 90° C. The reaction was monitored by LCMS. Quenched with water (10 mL) and extracted with EA (3×10 mL). The combined extracts were washed with water, brine, dried over Na₂SO₄, filtered, and concentrated. The crude product was purified by silica gel column chromatography, eluted with PE/EA (1:1) to afford 3-tert-butyl-1-{1-[(2-cyano-3-methylphenyl)methyl]-2-oxo-3,4-dihydroquinolin-6-yl}urea (10.3 mg, 5.10%) as a white solid. MS: mass calcd. For C₂₃H₂₆N₄O₂: 390.21 m/z, found 391.15 [M+H]⁺. ¹H NMR (300 MHz, DMSO-d₆) δ 8.13 (s, 1H), 7.47 (t, J=7.8 Hz, 1H), 7.35-7.33 (m, 2H), 6.99-6.97 (m, 2H), 6.66 (d, J=8.7 Hz, 1H), 5.92 (s, 1H), 5.21 (s, 2H), 2.91 (t, J=7.8 Hz, 2H), 2.67 (m, 2H), 2.51 (s, 3H), 1.26 (s, 9H).

### Example 54: 1-(tert-butyl)-3-(1-(2-fluoro-3-(trifluoromethyl)benzyl)-2-oxo-1,2,3,4-tetrahydroquinolin-6-yl)urea

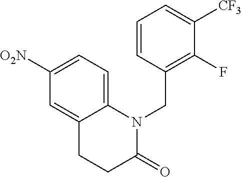 [SMILES: O=C1CCc2cc([N+](=O)[O-])ccc2N1Cc1cccc(C(F)(F)F)c1F]

### 1-(2-fluoro-3-(trifluoromethyl)benzyl)-6-nitro-3,4-dihydroquinolin-2(1H)-one

To a solution of To a solution of 6-nitro-3,4-dihydro-1H-quinolin-2-one (500 mg, 2.602 mmol, 1 equiv) in DMF (10 mL) was added K₂CO₃ (431.50 mg, 3.122 mmol, 2 equiv) and 1-(bromomethyl)-2-fluoro-3-(trifluoromethyl)benzene (601.86 mg, 2.341 mmol, 1.5 equiv). The resulting mixture was stirred for 16 h at rt. The mixture was diluted with EtOAc (50 mL), washed with water (3×20 mL), brine (2×20 mL) and concentrated under vacuum to yield a crude product which was directly purified by silica gel column, eluted with PE/EA (3:1) to afford 1-(2-fluoro-3-(trifluoromethyl)benzyl)-6-nitro-3,4-dihydroquinolin-2(1H)-one (450 mg, 78.27%) as a yellow solid. MS (ESI): mass calcd. for C₁₇H₁₂F₄N₂O₃: 368.08 m/z, found 369.05 [M+H]⁺.

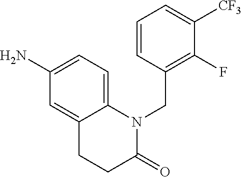 [SMILES: Nc1ccc2c(c1)CCC(=O)N2Cc1cccc(C(F)(F)F)c1F]

### 6-amino-1-(2-fluoro-3-(trifluoromethyl)benzyl)-3,4-dihydroquinolin-2(1H)-one

To a solution of 1-(2-fluoro-3-(trifluoromethyl)benzyl)-6-nitro-3,4-dihydroquinolin-2(1H)-one (430 mg, 1.168 mmol, 1 equiv) in MeOH (10 mL) was added Pd/C (100 mg). The reaction mixture was then stirred for 1 h under hydrogen atmosphere (balloon). The reaction mixture was filtered through a pad of Celite and concentrated under reduced pressure to afford 6-amino-1-(2-fluoro-3-(trifluoromethyl)benzyl)-3,4-dihydroquinolin-2(1H)-one (370 mg, 93.67%) as a yellow solid. MS (ESI): mass calcd. for C₁₇H₁₄F₄N₂O: 338.10 m/z, found 339.15 [M+H]⁺.

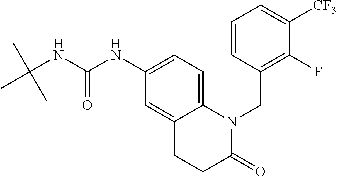 [SMILES: CC(C)(C)NC(=O)Nc1ccc2c(c1)CCC(=O)N2Cc1cccc(C(F)(F)F)c1F]

### 1-(tert-butyl)-3-(1-(2-fluoro-3-(trifluoromethyl)benzyl)-2-oxo-1,2,3,4-tetrahydroquinolin-6-yl)urea

To a solution of 6-amino-1-(2-fluoro-3-(trifluoromethyl)benzyl)-3,4-dihydroquinolin-2(1H)-one (150 mg, 0.443 mmol, 1 equiv) in DCM (10 mL) was added TEA (179.47 mg, 1.772 mmol, 4 equiv) and 2-isocyanato-2-methylpropane (219.77 mg, 2.215 mmol, 5 equiv). The resulting mixture was stirred for 16 h at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 1-(tert-butyl)-3-(1-(2-fluoro-3-(trifluoromethyl)benzyl)-2-oxo-1,2,3,4-tetrahydroquinolin-6-yl)urea (45.3 mg, 23.26%) as a white solid. MS (ESI): mass calcd. for C₂₂H₂₃F₄N₃O₂: 437.17, found: 437.95 [M+H]⁺. ¹H NMR (300 MHz, DMSO-d₆) δ 8.14 (s, 1H), 7.61-7.72 (m, 1H), 7.25-7.40 (m, 3H), 6.96-7.05 (m, 1H), 6.77 (d, J=8.8 Hz, 1H), 5.93 (s, 1H), 5.18 (s, 2H), 2.85-2.95 (m, 2H), 2.61-2.71 (m, 2H), 1.26 (s, 9H).

### Example 55: 3-tert-butyl-1-(1-{[2-cyano-5-(trifluoromethyl) phenyl]methyl}-2-oxo-3,4-dihydroquinolin-6-yl) urea

### Synthetic Scheme

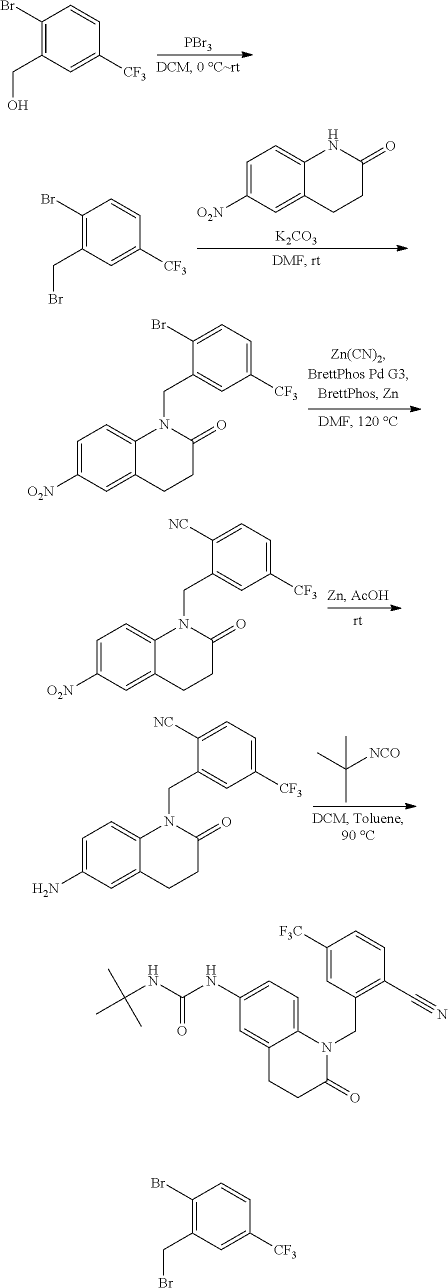

### 1-bromo-2-(bromomethyl)-4-(trifluoromethyl)benzene

To a stirred solution of [2-bromo-5-(trifluoromethyl) phenyl]methanol (2 g, 7.842 mmol, 1 equiv) in DCM (20 mL) was added phosphorus tribromide (2.12 g, 7.842 mmol, 1 equiv) in portions at 0° C. under air atmosphere. The resulting mixture was stirred for 3 h at room temperature under air atmosphere. The reaction was quenched with water (30 mL). The resulting mixture was extracted with ethyl acetate (3×30 mL). The organic layers were combined, dried over anhydrous sodium sulfate, filtered, and concentrated. This resulted in 1-bromo-2-(bromomethyl)-4-(trifluoromethyl) benzene (1.3 g, 52.14%) as a yellow oil. The crude product was used in the next step directly without further purification.

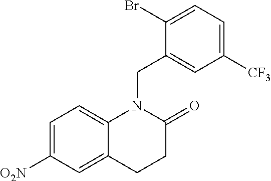 [SMILES: O=C1CCc2cc([N+](=O)[O-])ccc2N1Cc1cc(C(F)(F)F)ccc1Br]

### 1-{[2-bromo-5-(trifluoromethyl) phenyl]methyl}-6-nitro-3,4-dihydroquinolin-2-one

To a stirred solution of 1-bromo-2-(bromomethyl)-4-(trifluoromethyl) benzene (1.39 g, 4.372 mmol, 1.2 equiv) and 6-nitro-3,4-dihydro-1H-quinolin-2-one (700.16 mg, 3.643 mmol, 1.0 equiv) in DMF (15 mL) was added K₂CO₃ (1014.40 mg, 7.287 mmol, 2 equiv) in portions at room temperature under air atmosphere. The resulting mixture was stirred for 3 h at room temperature under air atmosphere. Desired product could be detected by LCMS. The reaction was quenched with water (30 mL). The resulting mixture was extracted with ethyl acetate (3×30 mL). The organic layers were combined, dried over anhydrous sodium sulfate, filtered, and concentrated. The residue was purified by silica gel column chromatography, eluted with PE/EA (1:1) to afford 1-{[2-bromo-5-(trifluoromethyl) phenyl]methyl}-6-nitro-3,4-dihydroquinolin-2-one (1.4 g, 89.76%) as a yellow oil. LC/MS: mass calcd. for C₁₇H₁₂BrF₃N₂O₃: 428.00, found: 428.95 [M+H]⁺.

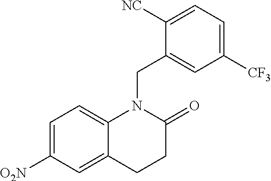 [SMILES: N#Cc1ccc(C(F)(F)F)cc1CN1C(=O)CCc2cc([N+](=O)[O-])ccc21]

### 2-[(6-nitro-2-oxo-3,4-dihydroquinolin-1-yl) methyl]-4-(trifluoromethyl)benzonitrile

To a stirred solution of 1-{[2-bromo-5-(trifluoromethyl) phenyl]methyl}-6-nitro-3,4-dihydroquinolin-2-one (600 mg, 1.398 mmol, 1 equiv) and Zn(CN)₂ (196.98 mg, 1.678 mmol, 1.2 equiv) in DMF (10 mL) was added BrettPhos Pd G3 (253.45 mg, 0.280 mmol, 0.2 equiv) and BrettPhos (150.08 mg, 0.280 mmol, 0.2 equiv) in portions at room temperature under air atmosphere. The resulting mixture was stirred for 3 h at 120° C. under nitrogen atmosphere. After cooling down to rt, the reaction was quenched with water (30 mL). The resulting mixture was extracted with ethyl acetate (3×30 mL). The organic layers were combined, dried over anhydrous sodium sulfate, filtered, and concentrated. The residue was purified by silica gel column chromatography, eluted with PE/EA (1:1) to afford 2-[(6-nitro-2-oxo-3,4-dihydroquinolin-1-yl) methyl]-4-(trifluoromethyl) benzonitrile (100 mg, 19.06%) as a yellow oil. Desired product could be detected by TLC.

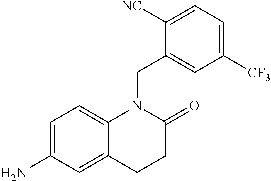 [SMILES: N#Cc1ccc(C(F)(F)F)cc1CN1C(=O)CCc2cc(N)ccc21]

### 2-[(6-amino-2-oxo-3,4-dihydroquinolin-1-yl) methyl]-4-(trifluoromethyl)benzonitrile

To a stirred solution of 2-[(6-nitro-2-oxo-3,4-dihydroquinolin-1-yl) methyl]-4-(trifluoromethyl) benzonitrile (100 mg, 0.266 mmol, 1 equiv) in AcOH was added Zn (209.04 mg, 3.192 mmol, 12 equiv) in portions at room temperature under air atmosphere. The resulting mixture was stirred for 3 h at room temperature under air atmosphere. Desired product could be detected by LCMS. The reaction was quenched with water (30 mL). The resulting mixture was extracted with ethyl acetate (3×30 mL). The organic layers were combined, dried over anhydrous sodium sulfate, filtered, and concentrated. The residue was purified by silica gel column chromatography, eluted with PE/EA (1:1) to afford 2-[(6-amino-2-oxo-3,4-dihydroquinolin-1-yl) methyl]-4-(trifluoromethyl) benzonitrile (85 mg, 92.54%) as an off-white solid. LC/MS: mass calcd. for C₁₈H₁₄F₃N₃O: 345.11, found: 346.05 [M+H]⁺.

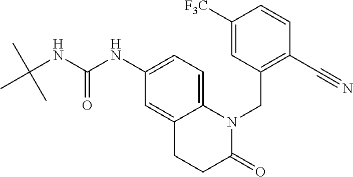 [SMILES: CC(C)(C)NC(=O)Nc1ccc2c(c1)CCC(=O)N2Cc1cc(C(F)(F)F)ccc1C#N]

### 3-tert-butyl-1-(1-{[2-cyano-5-(trifluoromethyl) phenyl]methyl}-2-oxo-3,4-dihydroquinolin-6-yl) urea

To a stirred solution of 2-[(6-amino-2-oxo-3,4-dihydroquinolin-1-yl) methyl]-4-(trifluoromethyl) benzonitrile (100 mg, 0.290 mmol, 1 equiv) in DCM/Toluene (10 mL, 2/1) was added 2-isocyanato-2-methylpropane (114.83 mg, 1.160 mmol, 4 equiv) in portions at room temperature under air atmosphere. The resulting mixture was stirred for 3 h at 90° C. under air atmosphere. Desired product could be detected by LCMS. The reaction was quenched with water (30 mL). The resulting mixture was extracted with ethyl acetate (3×30 mL). The organic layers were combined, dried over anhydrous sodium sulfate, filtered, and concentrated. The crude product was purified by Prep-HPLC with the following conditions to afford 3-tert-butyl-1-(1-{[2-cyano-5-(trifluoromethyl) phenyl]methyl}-2-oxo-3,4-dihydroquinolin-6-yl) urea (20.3 mg, 15.49%) as a white solid. MS (ESI): mass calcd. for C₂₃H₂₃F₃N₄O₂: 444.2m/z, found 445.10 [M+H]⁺. ¹H NMR (300 MHz, DMSO-d₆) δ 8.09-8.19 (m, 2H), 7.86 (d, J=8.1 Hz, 1H), 7.55 (s, 1H), 7.33 (d, J=2.4 Hz, 1H), 7.03 (dd, J=8.8, 2.4 Hz, 1H), 6.77 (d, J=8.7 Hz, 1H), 5.94 (s, 1H), 5.33 (s, 2H), 2.92 (t, J=7.3 Hz, 2H), 2.67 (t, J=7.2 Hz, 2H), 1.27 (s, 9H). ¹⁹F NMR (282 MHz, DMSO) δ −62.00.

### Example 56: (S)-1-(tert-butyl)-3-(2-oxo-1-(1-(m-tolyl)ethyl)-1,2,3,4-tetrahydroquinolin-6-yl)urea

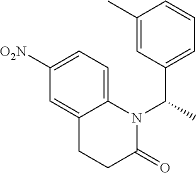 [SMILES: Cc1cccc([C@H](C)N2C(=O)CCc3cc([N+](=O)[O-])ccc32)c1]

### (S)-6-nitro-1-(1-(m-tolyl)ethyl)-3,4-dihydroquinolin-2(1H)-one

To a solution of (1R)-1-(3-methylphenyl)ethanol (300 mg, 2.203 mmol, 1 equiv) in DCM (5 mL) was added 6-nitro-3,4-dihydro-1H-quinolin-2-one (465.64 mg, 2.423 mmol, 1.1 equiv) and PPh₃ (577.76 mg, 2.203 mmol, 1 equiv). DEAD (383.62 mg, 2.203 mmol, 1 equiv) was added the mixture at 0° C. under a nitrogen atmosphere. The resulting mixture was stirred for 16 h at rt. The reaction mixture was quenched by water and extracted with EA (3×50 mL). The combined organic extracts were washed with brine (50 mL), dried over anhydrous Na₂SO₄, and concentrated under vacuum to yield a crude product which was directly purified by silica gel column, eluted with PE/EA (3:1) to afford (S)-6-nitro-1-(1-(m-tolyl)ethyl)-3,4-dihydroquinolin-2(1H)-one (310 mg, 45.35%) as a yellow solid. MS (ESI): mass calcd. for C₁₈H₁₈N₂O₃: 310.353, found: 311.10 [M+H]⁺.

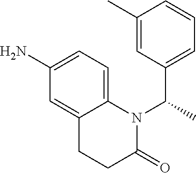 [SMILES: Cc1cccc([C@H](C)N2C(=O)CCc3cc(N)ccc32)c1]

### (S)-6-amino-1-(1-(m-tolyl)ethyl)-3,4-dihydroquinolin-2(1H)-one

To a solution of (S)-6-nitro-1-(1-(m-tolyl)ethyl)-3,4-dihydroquinolin-2(1H)-one (200 mg, 0.644 mmol, 1 equiv) in MeOH (10 mL) was added Pd/C (30 mg). The reaction mixture was then stirred for 1 h under hydrogen atmosphere (balloon). The reaction mixture was filtered through a pad of Celite and concentrated under reduced pressure to afford (S)-6-amino-1-(1-(m-tolyl)ethyl)-3,4-dihydroquinolin-2(1H)-one (170 mg, 94.44%) as a yellow solid. MS (ESI): mass calcd. For C₁₈H₂₀N₂O, 280.16 m/z, found 281.15 [M+H]⁺.

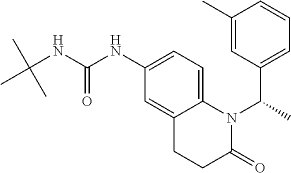 [SMILES: Cc1cccc([C@H](C)N2C(=O)CCc3cc(NC(=O)NC(C)(C)C)ccc32)c1]

### (S)-1-(tert-butyl)-3-(2-oxo-1-(1-(m-tolyl)ethyl)-1,2,3,4-tetrahydroquinolin-6-yl)urea

To a stirred solution of (S)-6-amino-1-(1-(m-tolyl)ethyl)-3,4-dihydroquinolin-2(1H)-one (100 mg, 0.357 mmol, 1 equiv) in DCM (5 mL) was added 2-isocyanato-2-methylpropane (176.79 mg, 1.785 mmol, 5 equiv) and TEA (144.37 mg, 1.428 mmol, 4 equiv) stirred overnight at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 26% B to 42% B in 10 min, 42% B; Wave Length: 254/220 nm; RT1(min): 8.6; Number Of Runs: 0) to afford (S)-1-(tert-butyl)-3-(2-oxo-1-(1-(m-tolyl)ethyl)-1,2,3,4-tetrahydroquinolin-6-yl)urea (20.4 mg, 15.03%) as a white solid. MS (ESI): mass calcd. for C₂₃H₂₉N₃O₂: 379.23 m/z, found 380.10 [M+H]⁺. ¹H NMR (300 MHz, DMSO-d₆) δ 8.07 (s, 1H), 7.32 (d, J=2.5 Hz, 1H), 7.22 (t, J=7.5 Hz, 1H), 7.01-7.11 (m, 3H), 6.78-6.87 (m, 1H), 6.44-6.53 (m, 1H), 6.06-6.20 (m, 1H), 5.93 (s, 1H), 2.73-2.88 (m, 2H), 2.52-2.73 (m, 2H), 2.29 (s, 3H), 1.66 (d, J=7.2 Hz, 3H), 1.26 (s, 9H).

### Example 57: (S)-1-(tert-butyl)-3-(3-oxo-4-(1-(m-tolyl)ethyl)-3,4-dihydro-2H-benzo[b][1,4]oxazin-7-yl)urea

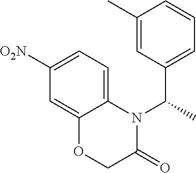 [SMILES: Cc1cccc([C@H](C)N2C(=O)COc3cc([N+](=O)[O-])ccc32)c1]

### (S)-7-nitro-4-(1-(m-tolyl)ethyl)-2H-benzo[b][1,4]oxazin-3(4H)-one

To a solution of (R)-1-(m-tolyl)ethan-1-ol (500 mg, 3.671 mmol, 1 equiv), 7-nitro-2H-benzo[b][1,4]oxazin-3(4H)-one (784.03 mg, 4.038 mmol, 1.1 equiv) and PPh₃ (1.44 g, 5.506 mmol, 1.5 equiv) in DCM (20 mL) was added DEAD (958.87 mg, 5.506 mmol, 1.5 equiv) under N₂ at 0° C. The reaction was stirred for 16 h at rt. The mixture was diluted with EtOAc (50 mL), washed with water (30 mL) and brine (30 mL), dried over anhydrous Na₂SO₄, and concentrated under vacuum to yield a crude product which was directly purified by silica gel column, eluted with PE/EA (3:1) to afford (S)-7-nitro-4-(1-(m-tolyl)ethyl)-2H-benzo[b][1,4]oxazin-3(4H)-one (620 mg, 54.07%) as a yellow oil. LC/MS: C₁₇H₁₆N₂O₄: 312.325, found: 313.10 [M+H]⁺.

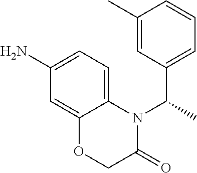 [SMILES: Cc1cccc([C@H](C)N2C(=O)COc3cc(N)ccc32)c1]

### (S)-7-amino-4-(1-(m-tolyl)ethyl)-2H-benzo[b][1,4]oxazin-3(4H)-one

To a solution of(S)-7-nitro-4-(1-(m-tolyl)ethyl)-2H-benzo[b][1,4]oxazin-3(4H)-one (300 mg, 0.961 mmol, 1 equiv) in MeOH (10 mL) was added Pd/C (50 mg). The reaction mixture was then stirred for 1 h under hydrogen atmosphere (balloon). The reaction mixture was filtered through a pad of Celite and concentrated under reduced pressure to afford (S)-7-amino-4-(1-(m-tolyl)ethyl)-2H-benzo[b][1,4]oxazin-3(4H)-one (200 mg, 73.75%) as a white solid. MS (ESI): mass calcd. for C₁₇H₁₈N₂O₂, 282.14 m/z, found 283.20 [M+H]⁺.

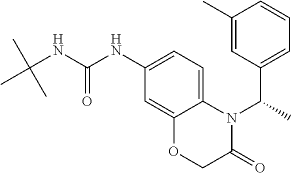 [SMILES: Cc1cccc([C@H](C)N2C(=O)COc3cc(NC(=O)NC(C)(C)C)ccc32)c1]

### (S)-1-(tert-butyl)-3-(3-oxo-4-(1-(m-tolyl)ethyl)-3,4-dihydro-2H-benzo[b][1,4]oxazin-7-yl)urea

To a stirred solution of (S)-7-amino-4-(1-(m-tolyl)ethyl)-2H-benzo[b][1,4]oxazin-3(4H)-one (200 mg, 0.708 mmol, 1 equiv) in DCM (10 mL) was added 2-isocyanato-2-methylpropane (351.1 mg, 3.542 mmol, 5 equiv) and TEA (215.0 mg, 2.125 mmol, 3 equiv) stirred 16 h at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 37% B to 57% B in 10 min; Wave Length: 220 nm; RT1(min): 9.5; Number Of Runs: 2) to afford (S)-1-(tert-butyl)-3-(3-oxo-4-(1-(m-tolyl)ethyl)-3,4-dihydro-2H-benzo[b][1,4]oxazin-7-yl)urea (44.8 mg, 16.56%) as a white solid. MS (ESI): mass calcd. for C₂₂H₂₇N₃O₃, 381.21 m/z, found 382.15 [M+H]⁺. ¹H NMR (300 MHz, DMSO-d₆) δ 8.18 (s, 1H), 7.20-7.28 (m, 2H), 7.10 (dd, J=14.1, 6.8 Hz, 3H), 6.62 (dd, J=8.9, 2.4 Hz, 1H), 6.55 (d, J=8.8 Hz, 1H), 6.09 (q, J=7.2 Hz, 1H), 5.94 (s, 1H), 4.57-4.75 (m, 2H), 2.30 (s, 3H), 1.70 (d, J=7.2 Hz, 3H), 1.25 (s, 9H).

### Example 58: 3-tert-butyl-1-{1-[(1S)-1-(2-fluorophenyl)ethyl]-2-oxo-3,4-dihydroquinolin-6-yl}ure

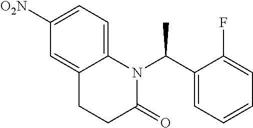 [SMILES: C[C@@H](c1ccccc1F)N1C(=O)CCc2cc([N+](=O)[O-])ccc21]

### 1-[(1S)-1-(2-fluorophenyl)ethyl]-6-nitro-3,4-dihydroquinolin-2-one

To a solution of (1R)-1-(2-fluorophenyl)ethanol (500 mg, 3.567 mmol, 1 equiv) and 6-nitro-3,4-dihydro-1H-quinolin-2-one (685.57 mg, 3.567 mmol, 1 equiv) in DCM (20 mL) was added triphenylphosphine (935.71 mg, 3.567 mmol, 1 equiv). The reaction was stirred at rt under N₂ for 0.5 hour. To the above mixture was added DEAD (621.29 mg, 3.567 mmol, 1 equiv) dropwise at 0° C. The reaction was stirred at rt under N₂ for 3h. Quenched with water (20 mL) and extracted with EA (3×30 mL). The combined extracts were washed with water, brine, dried over Na₂SO₄, filtered, and concentrated. The residue was purified by column chromatography on silica gel with EA/PE (0-100%) to give 1-[(1S)-1-(2-fluorophenyl)ethyl]-6-nitro-3,4-dihydroquinolin-2-one (723 mg, 64.48%) as a white solid. MS (ESI): mass calcd for C₁₇H₁₅FN₂O₃: 314.11 m/z, found 315.00[M+H]⁺.

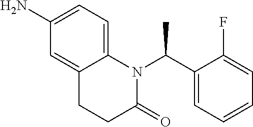 [SMILES: C[C@@H](c1ccccc1F)N1C(=O)CCc2cc(N)ccc21]

### 6-amino-1-[(1S)-1-(2-fluorophenyl)ethyl]-3,4-dihydroquinolin-2-one

To a solution of 1-[(1S)-1-(2-fluorophenyl)ethyl]-6-nitro-3,4-dihydroquinolin-2-one (710 mg, 2.259 mmol, 1 equiv) in methanol (20 mL) was added Pd/C (710 mg). The resulting mixture was stirred for 1 h at room temperature under hydrogen atmosphere. Desired product could be detected by LCMS. The resulting mixture was filtered, the filter cake was washed with MeOH (3×10 mL). The filtrate was concentrated under reduced pressure. The residue was purified by column chromatography on silica gel with EA/PE (0-100%) to give 6-amino-1-[(1S)-1-(2-fluorophenyl)ethyl]-3,4-dihydroquinolin-2-one (420 mg, 65.39%) as a black liquid. MS (ESI): mass calcd for C₁₇H₁₇FN₂O: 284.13m/z, found 285.10 [M+H]⁺.

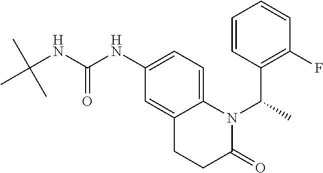 [SMILES: C[C@@H](c1ccccc1F)N1C(=O)CCc2cc(NC(=O)NC(C)(C)C)ccc21]

### 3-tert-butyl-1-{1-[(1S)-1-(2-fluorophenyl)ethyl]-2-oxo-3,4-dihydroquinolin-6-yl}urea

To a solution of 6-amino-1-[(1S)-1-(2-fluorophenyl)ethyl]-3,4-dihydroquinolin-2-one (200 mg, 0.703 mmol, 1 equiv) and 2-isocyanato-2-methylpropane (139.46 mg, 1.406 mmol, 2 equiv) in DCM (20 mL,) was added TEA (213.54 mg, 2.109 mmol, 3.00 equiv). The reaction was stirred at rt for 16h. Quenched with water (20 mL) and extracted with EA (3×30 mL). The combined extracts were washed with water, brine, dried over Na₂SO₄, filtered, and concentrated. The residue was purified by preparative HPLC using a XBridge Prep OBD C18 Column150 mm×30 mm×5 μm (eluent: 17% to 42% (v/v) CH₃CN and H₂O with 10 mmol/L NH₄HCO₃) to afford the title compound 3-tert-butyl-1-{1-[(1S)-1-(2-fluorophenyl)ethyl]-2-oxo-3,4-dihydroquinolin-6-yl}urea (21.2 mg, 7.85%) as a white solid. LC/MS (ESI): mass calcd. for C₂₂H₂₆FN₃O₂:383.20 m/z, found:384.00 [M+H]⁺. ¹H NMR (400 MHz, DMSO-d₆) δ 8.07 (s, 1H), 7.46-7.55 (m, 1H), 7.21-7.33 (m, 2H), 7.17 (m, 1H), 7.06 (m, 1H), 6.93 (m, 1H), 6.81 (m, 1H), 6.00 (m, 1H), 5.89 (s, 1H), 2.69-2.79 (m, 2H), 2.40-2.50 (m, 2H), 1.76 (s, 3H), 1.26 (s, 9H). ¹⁹F NMR (376 MHz, DMSO) δ −116.16.

### Example 59: 1-(tert-butyl)-3-(1-(2-cyano-3-(trifluoromethyl)benzyl)-2-oxo-1,2,3,4-tetrahydroquinolin-6-yl)urea

### Synthetic Scheme

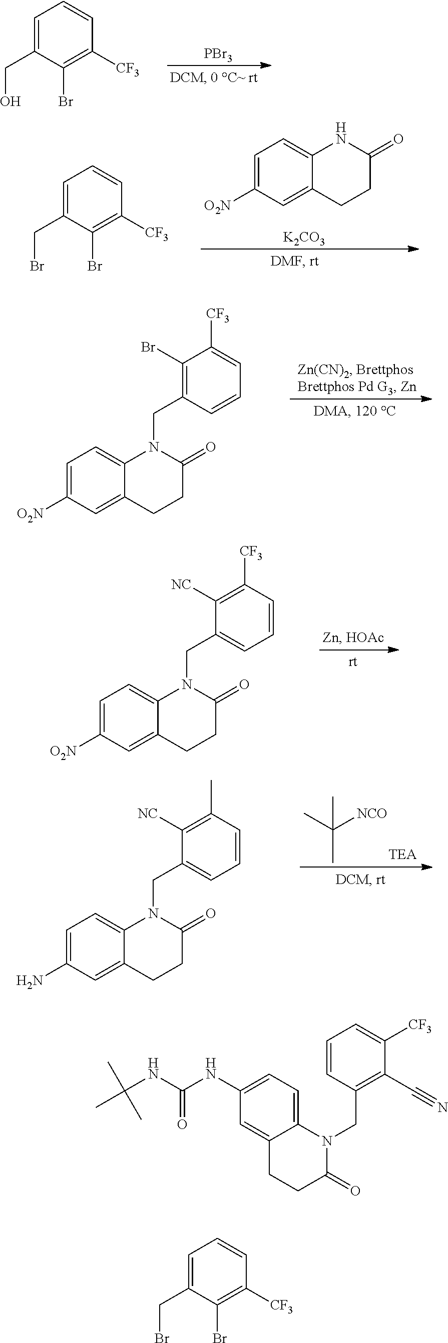

### 2-bromo-1-(bromomethyl)-3-(trifluoromethyl)benzene

To a solution of [2-bromo-3-(trifluoromethyl)phenyl]methanol (1.5 g, 5.882 mmol, 1 equiv) in DCM (10 mL) was added phosphorus tribromide (3.18 g, 11.764 mmol, 2 equiv) at 0° C. The reaction was stirred at rt for 2 hours. Quenched with water (10 mL) and extracted with EA (3×10 mL). The combined extracts were washed with water, brine, dried over Na₂SO₄, filtered, and concentrated. This resulted in 2-bromo-1-(bromomethyl)-3-(trifluoromethyl)benzene (1.83 g, 97.86%) as a yellow oil. MS (ESI): mass calcd. for C₈H₅Br₂F₃: 317.93m/z, found 318.90 [M+H]⁺.

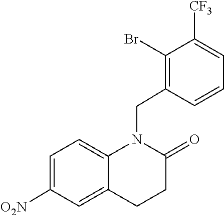 [SMILES: O=C1CCc2cc([N+](=O)[O-])ccc2N1Cc1cccc(C(F)(F)F)c1Br]

### 1-(2-bromo-3-(trifluoromethyl)benzyl)-6-nitro-3,4-dihydroquinolin-2(1H)-one

To a solution of 6-nitro-3,4-dihydro-1H-quinolin-2-one (426.14 mg, 2.217 mmol, 1 equiv) in DMF (10 mL) was added K₂CO₃ (617.40 mg, 4.434 mmol, 2 equiv) and 2-bromo-1-(bromomethyl)-3-(trifluoromethyl)benzene (705 mg, 2.217 mmol, 1 equiv). The reaction was stirred at rt for 1 hour. Quenched with water (20 mL) and extracted with EA (3×20 mL). The combined extracts were washed with water, brine, dried over Na₂SO₄, filtered, and concentrated. The residue was purified by column chromatography on silica gel with EA/PE (0-100%) to give 1-{[2-bromo-3-(trifluoromethyl)phenyl]methyl}-6-nitro-3,4-dihydroquinolin-2-one (500 mg, 52.54%) as a white solid. MS(ESI): mass calcd. for C₁₇H₁₂BrF₃N₂O₃: 429.19 m/z, found 428.95 [M+H]⁺.

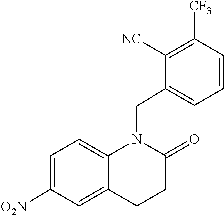 [SMILES: N#Cc1c(CN2C(=O)CCc3cc([N+](=O)[O-])ccc32)cccc1C(F)(F)F]

### 2-((6-nitro-2-oxo-3,4-dihydroquinolin-1(2H)-yl)methyl)-6-(trifluoromethyl)benzonitrile

To a solution of 1-{[2-bromo-3-(trifluoromethyl)phenyl]methyl}-6-nitro-3,4-dihydroquinolin-2-one (250 mg, 0.582 mmol, 1 equiv) in DMF (10 mL) was added Zn(CN)₂ (82.07 mg, 0.698 mmol, 1.2 equiv), Zn (19.04 mg, 0.291 mmol, 0.5 equiv), BrettPhos (62.53 mg, 0.116 mmol, 0.2 equiv) and BrettPhos Pd G3 (158.41 mg, 0.175 mmol, 0.3 equiv). The reaction was stirred at 120° C. under N₂ for 1 h. Quenched with water (10 mL) and extracted with EA (3×10 mL). The combined extracts were washed with water, brine, dried over Na₂SO₄, filtered, and concentrated. The residue was purified by column chromatography on silica gel with EA/PE (0-100%) to give 2-[(6-nitro-2-oxo-3,4-dihydroquinolin-1-yl)methyl]-6-(trifluoromethyl)benzonitrile (90 mg, 41.17%) as a white solid. MS (ESI): mass calcd. for C₁₈H₁₂F₃N₃O₃: 375.31 m/z, found:370.15 [M+H]⁺.

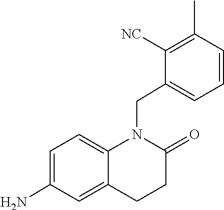 [SMILES: Cc1cccc(CN2C(=O)CCc3cc(N)ccc32)c1C#N]

### 2-((6-amino-2-oxo-3,4-dihydroquinolin-1(2H)-yl)methyl)-6-(trifluoromethyl)benzonitrile

To a solution of 2-[(6-nitro-2-oxo-3,4-dihydroquinolin-1-yl)methyl]-6-(trifluoromethyl)benzonitrile (140 mg, 0.373 mmol, 1 equiv) in HOAc (10 mL) was added zinc (243.89 mg, 3.730 mmol, 10 equiv). The reaction was stirred at rt for 1 hour. Quenched with water (10 mL) and extracted with EA (3×10 mL). The combined extracts were washed with water, brine, dried over Na₂SO₄, filtered, and concentrated. The residue was purified by column chromatography on silica gel with EA/PE (0-100%) to give 2-[(6-amino-2-oxo-3,4-dihydroquinolin-1-yl)methyl]-6-(trifluoromethyl)benzonitrile (90 mg, 69.87%) as a white solid. MS (ESI): mass calcd. for C₁₈H₁₄F₃N₃O: 345.33m/z, found:368.10 [M+Na]⁺.

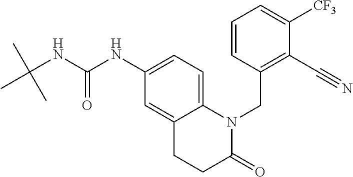 [SMILES: CC(C)(C)NC(=O)Nc1ccc2c(c1)CCC(=O)N2Cc1cccc(C(F)(F)F)c1C#N]

### 1-(tert-butyl)-3-(1-(2-cyano-3-(trifluoromethyl)benzyl)-2-oxo-1,2,3,4-tetrahydroquinolin-6-yl)urea

To a solution of 2-[(6-amino-2-oxo-3,4-dihydroquinolin-1-yl)methyl]-6-(trifluoromethyl)benzonitrile (90 mg, 0.261 mmol, 1 equiv) in DCM (10 mL) was added 2-isocyanato-2-methylpropane (103.35 mg, 1.044 mmol, 4 equiv) and TEA (79.12 mg, 0.783 mmol, 3 equiv). The reaction was stirred at rt for 1 day. Quenched with water (20 mL) and extracted with DCM (3×20 mL). The combined extracts were washed with water, brine, dried over Na₂SO₄, filtered, and concentrated. The residue was purified by preparative HPLC using a XBridge Prep OBD C18 Column, 30*150 mm, 5 μm column (eluent: 30% to 50% (v/v) CH₃CN and H₂O with 10 mmol/L NH₄HCO₃) to afford the title compound 3-tert-butyl-1-(1-{[2-cyano-3-(trifluoromethyl)phenyl]methyl}-2-oxo-3,4-dihydroquinolin-6-yl)urea (51 mg, 44.03%) as a white solid. MS (ESI): mass calcd. for C₂₃H₂₃F₃N₄O₂: 444.46m/z, found:445.15 [M+H]⁺. ¹H NMR (400 MHz, DMSO-d₆) δ 8.04-8.29 (m, 1H), 7.89-8.00 (m, 1H), 7.73-8.00 (m, 1H), 7.45-7.55 (m, 1H), 7.35-7.45 (m, 1H), 6.88-7.09 (m, 1H), 6.67-6.80 (m, 1H), 5.82-6.07 (m, 1H), 5.20-5.50 (m, 2H), 2.86-3.07 (m, 2H), 2.60-2.78 (m, 2H), 1.15-1.38 (m, 9H). ¹⁹F NMR (376 MHz, DMSO) δ (ppm): −60.5758.

### Example 60: (S)-3-tert-butyl-1-(2-oxo-1-{1-[3-(trifluoromethyl)phenyl]ethyl}-3,4-dihydroquinolin-6-yl)urea

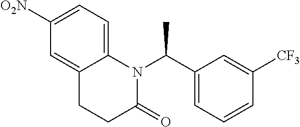 [SMILES: C[C@@H](c1cccc(C(F)(F)F)c1)N1C(=O)CCc2cc([N+](=O)[O-])ccc21]

### 6-nitro-1-[(1S)-1-[3-(trifluoromethyl)phenyl]ethyl]-3,4-dihydroquinolin-2-one

To a solution of (1R)-1-[3-(trifluoromethyl)phenyl]ethanol (500 mg, 2.629 mmol, 1 equiv) and 6-nitro-3,4-dihydro-1H-quinolin-2-one (505.28 mg, 2.629 mmol, 1 equiv) in DCM (20 mL) was added triphenylphosphine (689.64 mg, 2.629 mmol, 1 equiv). The reaction was stirred at rt under N₂ for 0.5 hour. To the above mixture was added DEAD (457.91 mg, 2.629 mmol, 1 equiv) dropwise at 0° C. The reaction was stirred at rt under N₂ for 3h. Quenched with water (30 mL) and extracted with EA (3×30 mL). The combined extracts were washed with water, brine, dried over Na₂SO₄, filtered and concentrated. The residue was purified by column chromatography on silica gel with EA/PE (0-100%) to give 6-nitro-1-[(1S)-1-[3-(trifluoromethyl)phenyl]ethyl]-3,4-dihydroquinolin-2-one (285 mg, 29.75%) as a white solid. MS (ESI): mass calcd. for C₁₈H₁₅F₃N₂O₃: 364.10 m/z, found 365.11[M+H]⁺.

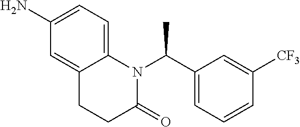 [SMILES: C[C@@H](c1cccc(C(F)(F)F)c1)N1C(=O)CCc2cc(N)ccc21]

### 6-amino-1-[(1S)-1-[3-(trifluoromethyl)phenyl]ethyl]-3,4-dihydroquinolin-2-one

To a solution of 6-nitro-1-[(S)-1-[3-(trifluoromethyl)phenyl]ethyl]-3,4-dihydroquinolin-2-one (285 mg, 0.782 mmol, 1 equiv) in methanol (3 mL) was added Pd/C (150 mg). The reaction was stirred at rt under H₂ for 1 h. The resulting mixture was stirred for 1 h at room temperature under hydrogen atmosphere. Desired product could be detected by LCMS. The resulting mixture was filtered, the filter cake was washed with MeOH (3×10 mL). The filtrate was concentrated under reduced pressure. The residue was purified by column chromatography on silica gel with EA/PE (0-100%) to give 6-amino-1-[(1S)-1-[3-(trifluoromethyl)phenyl]ethyl]-3,4-dihydroquinolin-2-one (225 mg, 86.03%) as a white solid. MS (ESI): mass calcd for C₁₈H₁₇F₃N₂O: 334.13 m/z, found 335.15 [M+H]⁺.

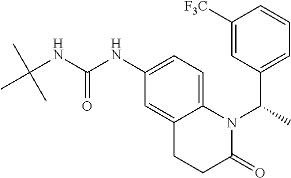 [SMILES: C[C@@H](c1cccc(C(F)(F)F)c1)N1C(=O)CCc2cc(NC(=O)NC(C)(C)C)ccc21]

### (S)-3-tert-butyl-1-(2-oxo-1-{1-[3-(trifluoromethyl)phenyl]ethyl}-3,4-dihydroquinolin-6-yl)urea

To a solution of 6-amino-1-[(1S)-1-[3-(trifluoromethyl)phenyl]ethyl]-3,4-dihydroquinolin-2-one (200 mg, 0.598 mmol, 1 equiv) and 2-isocyanato-2-methylpropane (177.90 mg, 1.794 mmol, 3 equiv) in DCM (3 mL) was added TEA (181.60 mg, 1.794 mmol, 3 equiv). The reaction was stirred at rt for 16h. Quenched with water (10 mL) and extracted with EA (3×10 mL). The combined extracts were washed with water, brine, dried over Na₂SO₄, filtered and concentrated. The residue was purified by preparative HPLC using a XBridge Prep OBD C18 150 mm×30 mm×5 μm column (eluent: 26% to 42% (v/v) CH₃CN and H₂O with 10 mmol/L NH₄HCO₃) to afford the title compound (S)-3-tert-butyl-1-(2-oxo-1-{1-[3-(trifluoromethyl)phenyl]ethyl}-3,4-dihydroquinolin-6-yl)urea (20.1 mg, 7.73%) as a white solid. LC/MS (ESI): mass calcd. for C₂₃H₂₆F₃N₃O₂:433.20 m/z, found:432.05 [M+H]⁺. ¹H NMR (300 MHz, DMSO-d₆) δ 8.10 (s, 1H), 7.52-7.65 (m, 4H), 7.33 (s, 1H), 6.90 (m, 1H), 6.52 (m, 1H), 6.08 (m, 1H), 5.92 (s, 1H), 2.79-2.89 (m, 2H), 2.52-2.66 (m, 2H), 1.74 (m, 3H), 1.26 (s, 9H). ¹⁹F NMR (282 MHz, DMSO) δ-60.93.

### Example 61: (S)-1-(tert-butyl)-3-(1-(1-(2-cyanophenyl)ethyl)-2-oxo-1,2,3,4-tetrahydroquinolin-6-yl)urea

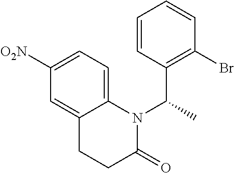 [SMILES: C[C@@H](c1ccccc1Br)N1C(=O)CCc2cc([N+](=O)[O-])ccc21]

### (S)-1-(1-(2-bromophenyl)ethyl)-6-nitro-3,4-dihydroquinolin-2(1H)-one

To a solution of (1R)-1-(2-bromophenyl)ethanol (1 g, 4.974 mmol, 1 equiv) in THF (10 mL) was added 6-nitro-3,4-dihydro-1H-quinolin-2-one (1.05 g, 5.471 mmol, 1.1 equiv), PPh₃ (1.30 g, 4.974 mmol, 1 equiv) at rt. The resulting mixture was stirred at 0° C. under a nitrogen atmosphere, which was added DEAD (0.87 g, 4.974 mmol, 1 equiv). The resulting mixture was extracted with EA (3×100 mL). The combined organic layers were washed with brine (50 mL), dried over anhydrous Na₂SO₄, and concentrated under vacuum to yield a crude product which was directly purified by flash chromatography (PE/EA=3:1) to afford (S)-1-(1-(2-bromophenyl)ethyl)-6-nitro-3,4-dihydroquinolin-2(1H)-one (1.2 g, 64.30%) as a yellow oil. MS (ESI): mass calcd. for C₁₇H₁₅BrN₂O₃: 374.03, found: 375.05[M+H]⁺.

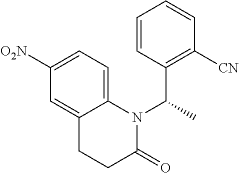 [SMILES: C[C@@H](c1ccccc1C#N)N1C(=O)CCc2cc([N+](=O)[O-])ccc21]

### (S)-2-(1-(6-nitro-2-oxo-3,4-dihydroquinolin-1(2H)-yl)ethyl)benzonitrile

A solution of 1-[(1S)-1-(2-bromophenyl)ethyl]-6-nitro-3,4-dihydroquinolin-2-one (400 mg, 1.066 mmol, 1 equiv) in dimethylformamide (10 mL) was added Zn(CN)₂ (250.35 mg, 2.132 mmol, 2.0 equiv) and Pd(PPh₃)₄ (123.19 mg, 0.107 mmol, 0.1 equiv) at rt. The resulting mixture was stirred for overnight at 110° C. The resulting mixture was extracted with EA (3×10 mL). The combined organic layers were washed with NaCl (3×5 mL), dried over anhydrous Na₂SO₄. After filtration, the filtrate was concentrated under reduced pressure. The residue was purified by silica gel column chromatography, eluted with PE/EA (3/1) to afford (S)-2-(1-(6-nitro-2-oxo-3,4-dihydroquinolin-1(2H)-yl)ethyl)benzonitrile (78 mg, 22.77%) as an off-white solid. MS (ESI): mass calcd. for C₁₈H₁₅N₃O₃, 321.11 m/z, found 322.20 [M+H]⁺.

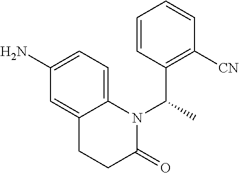 [SMILES: C[C@@H](c1ccccc1C#N)N1C(=O)CCc2cc(N)ccc21]

### (S)-2-(1-(6-amino-2-oxo-3,4-dihydroquinolin-1(2H)-yl)ethyl)benzonitrile

solution of (S)-2-(1-(6-nitro-2-oxo-3,4-dihydroquinolin-1(2H)-yl)ethyl)benzonitrile (70 mg, 0.218 mmol, 1 equiv) in MeOH (5 mL)/H₂O (0.5 mL) was added Fe (121.65 mg, 2.180 mmol, 10 equiv) and NH₄Cl (116.52 mg, 2.180 mmol, 10 equiv) at rt. The resulting mixture was stirred for 1 h at 60° C. The precipitated solids were collected by filtration and washed with MeOH (3×5 mL). The aqueous layer was extracted with DCM (3×5 mL). The organic layer was concentrated under reduced pressure. The residue was purified by silica gel column chromatography, eluted with PE/EA (2:1) to afford (S)-2-(1-(6-amino-2-oxo-3,4-dihydroquinolin-1(2H)-yl)ethyl)benzonitrile (60 mg, 94.53%) as a yellow solid. MS (ESI): mass calcd. for C₁₈H₁₇N₃O, 291.14 m/z, found 292.20 [M+H]⁺.

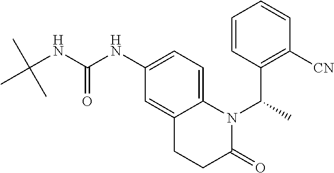 [SMILES: C[C@@H](c1ccccc1C#N)N1C(=O)CCc2cc(NC(=O)NC(C)(C)C)ccc21]

### (S)-1-(tert-butyl)-3-(1-(1-(2-cyanophenyl)ethyl)-2-oxo-1,2,3,4-tetrahydroquinolin-6-yl)urea

A solution of (S)-2-(1-(6-amino-2-oxo-3,4-dihydroquinolin-1(2H)-yl)ethyl)benzonitrile (70 mg, 0.240 mmol, 1 equiv) in DCM (5 mL) was added 2-isocyanato-2-methylpropane (119.09 mg, 1.200 mmol, 5.0 equiv) and TEA (97.25 mg, 0.960 mmol, 4.0 equiv) at rt. The resulting mixture was stirred for overnight at TR. The resulting mixture was extracted with EA (5 mL×3). The combined organic layers were washed with brine, dried over anhydrous Na₂SO₄. After filtration, the filtrate was concentrated under reduced pressure. The crude product 50 mg was purified by Prep-HPLC with the following conditions (Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 30% B to 54% B in 7 min; Wave Length: 220 nm; RT1(min): 6.5; Number Of Runs: 0) to afford (S)-1-(tert-butyl)-3-(1-(1-(2-cyanophenyl)ethyl)-2-oxo-1,2,3,4-tetrahydroquinolin-6-yl)urea (22.5 mg, 23.93%) as white solid. MS (ESI):mass calcd. C₂₃H₂₆N₄O₂ for:390.21 m/z, found 391.20 [M+H]⁺. ¹H NMR (400 MHz, Chloroform-d) δ 7.72 (d, J=7.9 Hz, 1H), 7.56-7.66 (m, 2H), 7.21-7.32 (m, 1H), 7.18-7.20 (m, 1H), 7.09-7.12 (m, 1H), 6.79-6.92 (m, 1H), 6.61-6.74 (m, 1H), 5.87-6.02 (m, 2H), 2.92-3.01 (m, 1H), 2.71-2.83 (m, 1H), 2.53-2.69 (m, 1H), 2.42-2.53 (m, 1H), 1.96 (d, J=7.1 Hz, 3H), 1.35-1.40 (m, 9H).

### Example 62: (S)-1-(tert-butyl)-3-(3-oxo-4-(1-(3-(trifluoromethyl)phenyl)ethyl)-3,4-dihydro-2H-benzo[b][1,4]oxazin-7-yl)urea

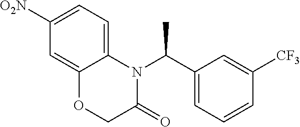 [SMILES: C[C@@H](c1cccc(C(F)(F)F)c1)N1C(=O)COc2cc([N+](=O)[O-])ccc21]

### (S)-7-nitro-4-(1-(3-(trifluoromethyl)phenyl)ethyl)-2H-benzo[b][1,4]oxazin-3(4H)-one

To a solution of (1R)-1-[3-(trifluoromethyl)phenyl]ethanol (500 mg, 2.629 mmol, 1 equiv), 7-nitro-2,4-dihydro-1,4-benzoxazin-3-one (510.47 mg, 2.629 mmol, 1 equiv) and PPh₃ (1034.46 mg, 3.944 mmol, 1.5 equiv) in DCM (10 mL). The reaction was stirred at rt under N₂ for 0.5 hour. To the above mixture was added DEAD (686.86 mg, 3.944 mmol, 1.5 equiv) dropwise at 0° C. The resulting mixture was stirred for additional 1 h at rt. Quenched with water (20 mL) and extracted with DCM (3×20 mL). The combined extracts were washed with water, brine, dried over Na₂SO₄, filtered, and concentrated. The residue was purified by column chromatography on silica gel with EA/PE (0-100%) to give 7-nitro-4-[(1S)-1-[3-(trifluoromethyl)phenyl]ethyl]-2H-1,4-benzoxazin-3-one (550 mg, 57.11%) as a yellow solid. MS (ESI): mass calcd. for C₁₇H₁₃F₃N₂O₄: 366.30m/z, found 367.00 [M+H]⁺.

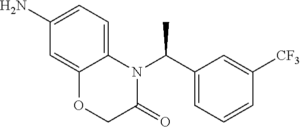 [SMILES: C[C@@H](c1cccc(C(F)(F)F)c1)N1C(=O)COc2cc(N)ccc21]

### (S)-7-amino-4-(1-(3-(trifluoromethyl)phenyl)ethyl)-2H-benzo[b][1,4]oxazin-3(4H)-one

To a solution of 7-nitro-4-[(1S)-1-[3-(trifluoromethyl)phenyl]ethyl]-2H-1,4-benzoxazin-3-one (500 mg, 1.365 mmol, 1 equiv) in MeOH/H₂O (16.5 mL, 10:1) was added Fe (762.29 mg, 13.650 mmol, 10 equiv) and NH₄Cl (730.15 mg, 13.650 mmol, 10 equiv). The reaction was stirred at 80° C. for 1 hour. The reaction was quenched with water at rt. After filtration, the filter cake was washed with EA (3×20 mL). The filtrate was added water (20 mL) and extracted with EA (3×20 mL). The combined extracts were washed with water, brine, dried over Na₂SO₄, filtered, and concentrated. This resulted in 7-amino-4-[(1S)-1-[3-(trifluoromethyl)phenyl]ethyl]-2H-1,4-benzoxazin-3-one (450 mg, 98.02%) as a yellow solid. MS (ESI): mass calcd. for C₁₇H₁₅F₃N₂O₂: 336.31m/z, found 337.15 [M+H]⁺.

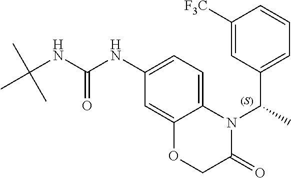

### (S)-1-(tert-butyl)-3-(3-oxo-4-(1-(3-(trifluoromethyl)phenyl)ethyl)-3,4-dihydro-2H-benzo[b][1,4]oxazin-7-yl)urea

To a solution of 7-amino-4-[(1S)-1-[3-(trifluoromethyl)phenyl]ethyl]-2H-1,4-benzoxazin-3-one (150 mg, 0.446 mmol, 1 equiv) in DCM (10 mL) was added 2-isocyanato-2-methylpropane (176.86 mg, 1.784 mmol, 4 equiv) and TEA (135.40 mg, 1.338 mmol, 3 equiv). The reaction was stirred at rt for 1 day. Quenched with water (20 mL) and extracted with EA (3×20 mL). The combined extracts were washed with water, brine, dried over Na₂SO₄, filtered, and concentrated. The residue was purified by preparative HPLC using a XBridge Prep OBD C18 Column, 30*150 mm, 5 m column (eluent: 35% to 60% (v/v) CH₃CN and H₂O with 10 mmol/L NH₄HCO₃) to afford the title compound 3-tert-butyl-1-{3-oxo-4-[(1S)-1-[3-(trifluoromethyl)phenyl]ethyl]-2H-1,4-benzoxazin-7-yl}urea (50.1 mg, 25.80%) as a white solid. MS (ESI): mass calcd. for C₂₂H₂₄F₃N₃O₃: 435.45 m/z, found:436.20 [M+H]⁺. ¹H NMR (400 MHz, DMSO-d6) δ 8.10-8.31 (m, 1H), 7.45-7.80 (m, 4H), 7.07-7.38 (m, 1H), 6.44-6.85 (m, 2H), 6.01-6.19 (m, 1H), 5.79-6.01 (m, 1H), 4.50-4.90 (m, 2H), 1.70-1.90 (m, 3H), 1.21-1.32 (m, 9H). ¹⁹F NMR (376 MHz, DMSO) δ (ppm): −60.8904.

### Example 63: 5-benzyl-6-oxo-7,8-dihydro-5H-naphthalen-2-yl N-tert-butylcarbamate

### Synthetic Scheme

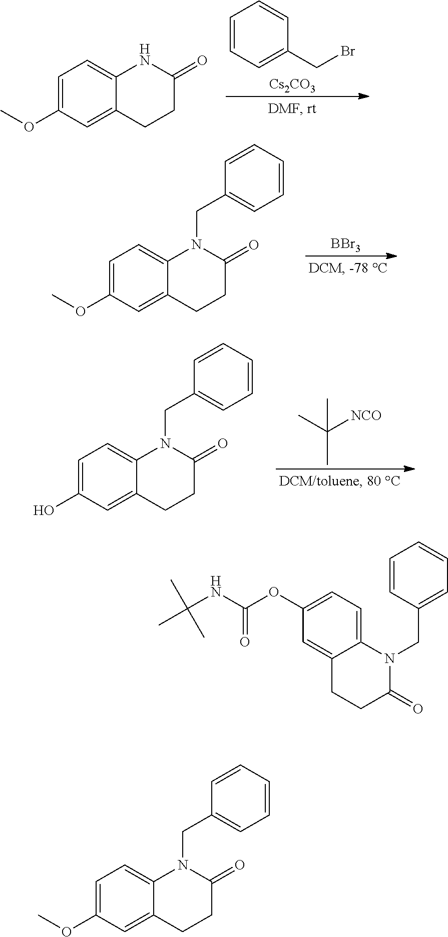

### 1-benzyl-6-methoxy-3,4-dihydroquinolin-2-one

To a solution of 6-methoxy-3,4-dihydro-1H-quinolin-2-one (473.5 mg, 2.672 mmol, 1 equiv) and benzyl bromide (1142.56 mg, 6.680 mmol, 2.5 equiv) in dimethylformamide (25 mL) was added Cs₂CO₃ (2619.92 mg, 8.016 mmol, 3 equiv). The reaction was stirred at rt for 3h. Quenched with water (50 mL) and extracted with EA (3×50 mL). The combined extracts were washed with water, brine, dried over Na₂SO₄, filtered, and concentrated. The residue was purified by column chromatography on silica gel with EA/PE (0-100%) to give tert-butyl 1-benzyl-6-methoxy-3,4-dihydroquinolin-2-one (640 mg, 89.60%) as a white solid. MS (ESI): mass calcd. for C₁₇H₁₇NO₂: 267.13 m/z, found 268.10[M+H]⁺.

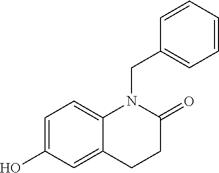 [SMILES: O=C1CCc2cc(O)ccc2N1Cc1ccccc1]

### 1-benzyl-6-hydroxy-3,4-dihydroquinolin-2-one

To a solution of 1-benzyl-6-methoxy-3,4-dihydroquinolin-2-one (620 mg, 2.319 mmol, 1 equiv) in DCM (8 mL) was added boron tribromide (7 mL) at −78° C. under nitrogen atmosphere. The reaction was stirred at −78° C. for 3h. Quenched with water (50 mL) and extracted with EA (3×50 mL). The combined extracts were washed with water, brine, dried over Na₂SO₄, filtered, and concentrated. The residue was purified by column chromatography on silica gel with EA/PE (0-100%) to give 1-benzyl-6-hydroxy-3,4-dihydroquinolin-2-one (312 mg, 53.11%) as a black solid. MS (ESI): mass calcd for C₁₆H₁₅NO₂: 253.11 m/z, found 253.95[M+H]⁺.

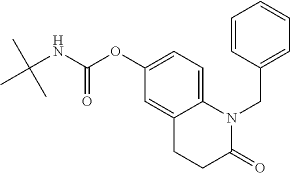 [SMILES: CC(C)(C)NC(=O)Oc1ccc2c(c1)CCC(=O)N2Cc1ccccc1]

### 5-benzyl-6-oxo-7,8-dihydro-5H-naphthalen-2-yl N-tert-butylcarbamate

To a solution of 1-benzyl-6-hydroxy-3,4-dihydro-1H-naphthalen-2-one (132 mg, 0.523 mmol, 1 equiv) and 2-isocyanato-2-methylpropane (103.32 mg, 1.042 mmol, 2 equiv) in DCM (2 mL) was added TEA (158.20 mg, 1.563 mmol, 3 equiv). The reaction was stirred at rt for 16h. Quenched with water (10 mL) and extracted with EA (3×10 mL). The combined extracts were washed with water, brine, dried over Na₂SO₄, filtered, and concentrated. The residue was purified by preparative HPLC using a XBridge Prep OBD C18 150 mm×30 mm×5 μm column (eluent: 17% to 42% (v/v) CH₃CN and H₂O with 10 mmol/L NH₄HCO₃) to afford the title compound 5-benzyl-6-oxo-7,8-dihydro-5H-naphthalen-2-yl N-tert-butylcarbamate (50.2 mg, 27.27%) as a white solid. LC/MS (ESI): mass calcd. for C₂₁H₂₄N₂O₃:352.18 m/z, found:353.05 [M+H]⁺. ¹H NMR (300 MHz, DMSO-d₆) δ 7.50 (s, 1H), 7.22-7.37 (m, 5H), 6.97 (m, 1H), 6.76-6.91 (m, 2H), 5.13 (s, 2H), 2.89-3.01 (m, 2H), 2.69-2.73 (m, 2H), 1.25 (s, 9H).

### Example 64: 1-(tert-butyl)-3-(1-(cyclohexylmethyl)-2-oxo-1,2,3,4-tetrahydroquinolin-6-yl)urea

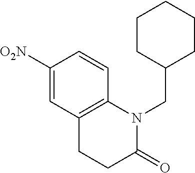 [SMILES: O=C1CCc2cc([N+](=O)[O-])ccc2N1CC1CCCCC1]

### 1-(cyclohexylmethyl)-6-nitro-3,4-dihydroquinolin-2(1H)-one

To a stirred solution of 6-nitro-3,4-dihydro-1H-quinolin-2-one (300 mg, 1.561 mmol, 1.0 equiv) in DMF (15 mL) was added Cs₂CO₃ (1.53 g, 4.683 mmol, 3.0 equiv) and (bromomethyl)cyclohexane (276.44 mg, 1.561 mmol, 1 equiv) at rt and stirred for overnight. The reaction progress was monitored by LCMS. After completion of reaction, the resulting mixture was extracted with EA (3×30 mL). The combined organic layers were washed with water (3×20 mL), dried over anhydrous Na₂SO₄. After filtration, the filtrate was concentrated under reduced pressure. The residue was purified by silica gel column chromatography (PE/EA=3:1) to afford 1-(cyclohexylmethyl)-6-nitro-3,4-dihydroquinolin-2(1H)-one (310 mg, 68.87%) as a yellow solid. MS (ESI): mass calcd. for C₁₆H₂₀N₂O₃: 288.15 m/z, found 289.15 [M+H]⁺.

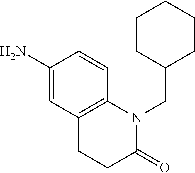 [SMILES: Nc1ccc2c(c1)CCC(=O)N2CC1CCCCC1]

### 6-amino-1-(cyclohexylmethyl)-3,4-dihydroquinolin-2(1H)-one

To a solution of 1-(cyclohexylmethyl)-6-nitro-3,4-dihydroquinolin-2-one (100 mg, 0.347 mmol, 1 equiv) in methanol (5 mL) was added Pd/C (30 mg) under nitrogen atmosphere. The mixture was hydrogenated at room temperature for 50 min under hydrogen atmosphere using a hydrogen balloon, filtered through a Celite pad and concentrated under reduced pressure. This resulted in 6-amino-1-(cyclohexylmethyl)-3,4-dihydroquinolin-2(1H)-one as a white solid. MS (ESI): mass calcd. for C₁₆H₂₂N₂O: 258.17 m/z, found 259.30 [M+H]⁺.

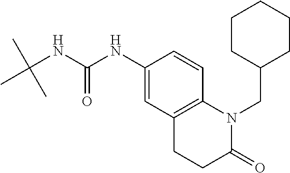 [SMILES: CC(C)(C)NC(=O)Nc1ccc2c(c1)CCC(=O)N2CC1CCCCC1]

### 1-(tert-butyl)-3-(1-(cyclohexylmethyl)-2-oxo-1,2,3,4-tetrahydroquinolin-6-yl)urea

A solution of 6-amino-1-(cyclohexylmethyl)-3,4-dihydroquinolin-2(1H)-one (50 mg, 0.194 mmol, 1 equiv), triethylamine (58.75 mg, 0.582 mmol, 3 equiv) and 2-isocyanato-2-methylpropane (57.55 mg, 0.582 mmol, 3 equiv) in DCM (5 mL) was stirred for overnight at rt. The residue was purified by silica gel column chromatography (PE/EA=2:1) to afford crude products. The residue was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 26% B to 50% B in 9 min, 50% B; Wave Length: 254/220 nm; RT1(min): 8.9;) to afford 1-(tert-butyl)-3-(1-(cyclohexylmethyl)-2-oxo-1,2,3,4-tetrahydroquinolin-6-yl)urea (26.5 mg, 38.18%) as a white solid. MS (ESI):mass calcd. for C₂₁H₃₁N₃O₂: 357.24 m/z, found 358.25 [M+H]⁺. ¹H NMR (300 MHz, DMSO-d₆) δ 8.14 (s, 1H), 7.28 (d, J=2.4 Hz, 1H), 7.12 (dd, J=8.8, 2.5 Hz, 1H), 6.99 (d, J=8.8 Hz, 1H), 5.94 (s, 1H), 3.76 (d, J=6.9 Hz, 2H), 2.77 (dd, J=8.8, 5.8 Hz, 2H), 2.47 (d, J=6.3 Hz, 2H), 1.55-1.64 (m, 6H), 1.28 (s, 9H), 1.09 (t, J=9.5 Hz, 3H), 0.95 (t, J=11.2 Hz, 2H).

### Example 65:1-(1-benzyl-7-fluoro-2-oxo-1,2,3,4-tetrahydroquinolin-6-yl)-3-(tert-butyl)urea

### Synthetic Scheme

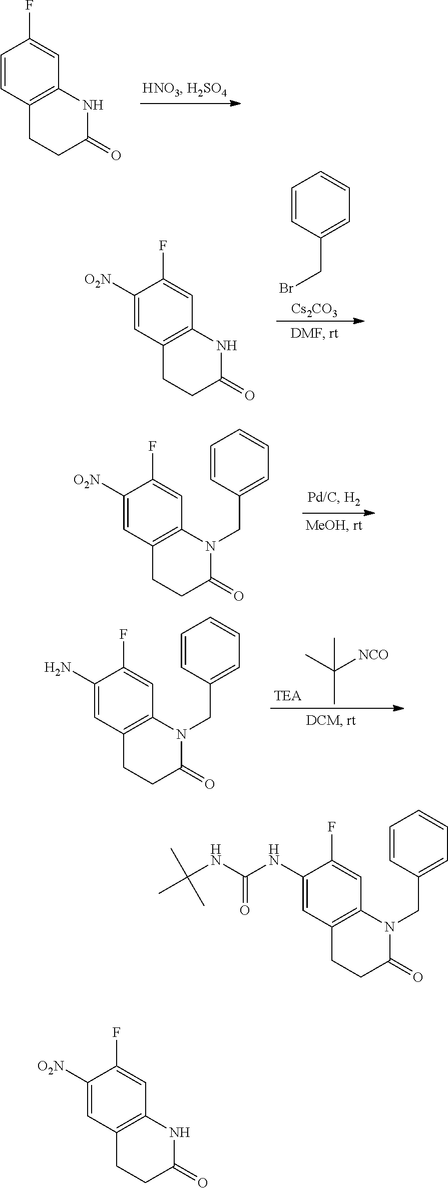

### 7-fluoro-6-nitro-3,4-dihydroquinolin-2(1H)-one

To a solution of 7-fluoro-3,4-dihydro-1H-quinolin-2-one (526 mg, 3.185 mmol, 1 equiv) in H₂SO₄ (3.2 mL) was added HNO₃ (200.6 mg, 3.185 mmol, 1.0 equiv) at 0° C. The resulting mixture was stirred at 0° C. until the starting material was totally consumed by LCMS, the reaction mixture was quenched by water and extracted with EA (3×20 mL). The combined organic extracts were washed with brine (20 mL), dried over anhydrous Na₂SO₄, and concentrated under vacuum to yield a crude product which was directly purified by flash chromatography (PE/EA=3:1) to afford 7-fluoro-6-nitro-3,4-dihydro-1H-quinolin-2-one (300 mg, 35.30%) as a yellow solid. MS (ESI): mass calcd. for C₉H₇FN₂O₃, 210.04 m/z, found 211.10 [M+H]⁺.

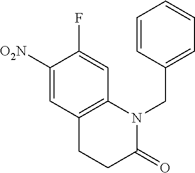 [SMILES: O=C1CCc2cc([N+](=O)[O-])c(F)cc2N1Cc1ccccc1]

### 1-benzyl-7-fluoro-6-nitro-3,4-dihydroquinolin-2(1H)-one

To a solution of 7-fluoro-6-nitro-3,4-dihydro-1H-quinolin-2-one (300 mg, 1.427 mmol, 1 equiv) in DMF (8 mL) was added K₂CO₃ (591.8 mg, 4.282 mmol, 3 equiv) and benzyl bromide (488.3 mg, 2.855 mmol, 2 equiv). The resulting mixture was placed at rt and stirred overnight until the starting material was totally consumed by LCMS. The reaction mixture was quenched by water and extracted with EA (3*20 mL). The combined organic extracts were washed with brine (20 mL), dried over anhydrous Na₂SO₄, and concentrated under vacuum to yield a crude product which was directly purified by flash chromatography (0-100% ethyl acetate/petroleum ether) to afford 1-benzyl-7-fluoro-6-nitro-3,4-dihydroquinolin-2-one (250 mg, 57.56%) as a white solid. MS (ESI): mass calcd. for C₁₆H₁₃FN₂O₃, 300.09 m/z, found 301.10 [M+H]⁺.

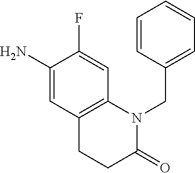 [SMILES: Nc1cc2c(cc1F)N(Cc1ccccc1)C(=O)CC2]

### 6-amino-1-benzyl-7-fluoro-3,4-dihydroquinolin-2(1H)-one

To a solution of 1-benzyl-7-fluoro-6-nitro-3,4-dihydroquinolin-2-one (250 mg, 0.833 mmol, 1 equiv) in MeOH (10 mL) was added Pd/C (30 mg). The mixture was hydrogenated at room temperature for 50 min under hydrogen atmosphere using a hydrogen balloon, then filtered through a Celite pad and concentrated under reduced pressure to afford 6-amino-1-benzyl-7-fluoro-3,4-dihydroquinolin-2-one (220 mg, 86.79%) as a yellow solid. MS (ESI): mass calcd. for C₁₆H₁₅FN₂O, 270.12 m/z, found 271.10 [M+H]⁺

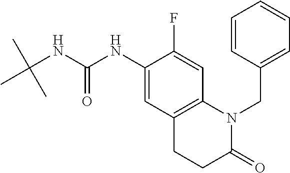 [SMILES: CC(C)(C)NC(=O)Nc1cc2c(cc1F)N(Cc1ccccc1)C(=O)CC2]

### 1-(1-benzyl-7-fluoro-2-oxo-1,2,3,4-tetrahydroquinolin-6-yl)-3-(tert-butyl)urea

To a stirred solution of 6-amino-1-benzyl-7-fluoro-3,4-dihydroquinolin-2-one (200 mg, 0.740 mmol, 1 equiv) in DCM (10.00 mL) was added 2-isocyanato-2-methylpropane (366.7 mg, 3.699 mmol, 5 equiv) and TEA (224.6 mg, 2.220 mmol, 3 equiv) stirred overnight at rt. The reaction progress was monitored by LCMS. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by flash chromatography (0-100% ethyl acetate/petroleum ether) to afford 100 mg crude product as a white solid. The compound was separated by preparative HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 32% B to 52% B in 10 min; Wave Length: 254 nm; RT1(min): 10.2; Number Of Runs: 3) to afford 1-(1-benzyl-7-fluoro-2-oxo-3,4-dihydroquinolin-6-yl)-3-tert-butylurea (34.5 mg, 12.51%) as a white solid. MS (ESI): mass calcd. for C₂₁H₂₄FN₃O₂, 369.19 m/z, found 370.20 [M+H]⁺. ¹H NMR (300 MHz, DMSO-d₆) δ 7.94 (dd, J=5.7, 3.2 Hz, 2H), 7.27-7.37 (m, 2H), 7.18-7.27 (m, 3H), 6.79 (d, J=13.3 Hz, 1H), 6.43 (s, 1H), 5.10 (s, 2H), 2.87 (t, J=7.2 Hz, 2H), 2.66 (dd, J=8.7, 5.8 Hz, 2H), 1.26 (s, 9H).

### Example 66: 1-(1-benzyl-7-chloro-2-oxo-3,4-dihydroquinolin-6-yl)-3-tert-butylurea

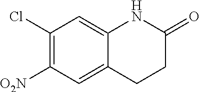 [SMILES: O=C1CCc2cc([N+](=O)[O-])c(Cl)cc2N1]

### Synthesis of 7-chloro-6-nitro-3,4-dihydro-1H-quinolin-2-one

To a stirred solution of 7-chloro-3,4-dihydro-1H-quinolin-2-one (2 g, 11.012 mmol, 1 equiv) in H₂SO₄ was added KNO₃ (1.11 g, 11.012 mmol, 1 equiv) at 0° C. The final reaction mixture was stirred overnight at room temperature. The reaction was monitored by LCMS. The reaction was poured into the Water/Ice (100 mL). The resulting mixture was extracted with EA (3×50 mL). The combined organic layers were washed with brine (2×20 mL), dried over anhydrous Na₂SO₄. After filtration, the filtrate was concentrated under reduced pressure. The crude product was used in the next step directly without further purification. MS: mass calcd. for C₉H₇ClN₂O₃: 226.01 m/z, found 227.00 [M+H]⁺.

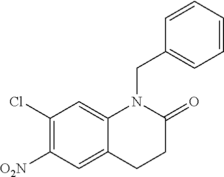 [SMILES: O=C1CCc2cc([N+](=O)[O-])c(Cl)cc2N1Cc1ccccc1]

### 1-benzyl-7-chloro-6-nitro-3,4-dihydroquinolin-2-one

To a stirred solution of 7-chloro-6-nitro-3,4-dihydro-1H-quinolin-2-one (1.8 g, 7.943 mmol, 1 equiv) in DMF (20 mL) was added benzyl bromide (2.72 g, 15.886 mmol, 2 equiv) and Cs₂CO₃ (5.19 g, 15.886 mmol, 2 equiv) at room temperature. The final reaction mixture was stirred for 3 h at room temperature. The reaction was monitored by LCMS. The resulting mixture was quenched with water (30 mL) and extracted with EA (3×30 mL). The combined organic layers were washed with brine (30 mL), dried over anhydrous Na₂SO₄. After filtration, the filtrate was concentrated under reduced pressure. The crude product was purified by silica gel column chromatography, eluted with PE/EA (1:1) to afford 1-benzyl-7-chloro-6-nitro-3,4-dihydroquinolin-2-one (1.3 g, 51.67%) as an orange solid. MS: mass calcd. for C₁₆H₁₃ClN₂O₃: 316.06 m/z, found 317.15 [M+H]⁺.

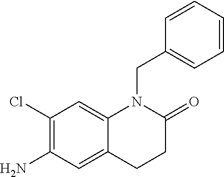 [SMILES: Nc1cc2c(cc1Cl)N(Cc1ccccc1)C(=O)CC2]

### 6-amino-1-benzyl-7-chloro-3,4-dihydroquinolin-2-one

To a stirred solution of 1-benzyl-7-chloro-6-nitro-3,4-dihydroquinolin-2-one (500 mg, 1.579 mmol, 1 equiv) in EtOH/H₂O (10 mL, 10/1) were added Fe (881.56 mg, 15.790 mmol, 10 equiv) and NH₄Cl (844.38 mg, 15.790 mmol, 10 equiv) at room temperature. The final reaction mixture was stirred for 3 h at 60° C. The reaction was monitored by LCMS. The reaction was quenched with water at rt. After filtration, the filter cake was washed with EA (3×20 mL). The filtrate was added water (20 mL) and extracted with EA (3×20 mL). The combined organic layers were washed with brine (30 mL), dried over anhydrous Na₂SO₄. After filtration, the filtrate was concentrated under reduced pressure. This resulted in 6-amino-1-benzyl-7-chloro-3,4-dihydroquinolin-2-one (300 mg, 66.27%) as a yellow solid. The crude product was used in the next step directly without further purification. MS (ESI): mass calcd. for C₁₆H₁₅ClN₂O: 286.09 m/z, found 287.05 [M+H]⁺.

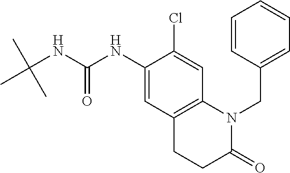 [SMILES: CC(C)(C)NC(=O)Nc1cc2c(cc1Cl)N(Cc1ccccc1)C(=O)CC2]

### 1-(1-benzyl-7-chloro-2-oxo-3,4-dihydroquinolin-6-yl)-3-tert-butylurea

To a stirred solution of 6-amino-1-benzyl-7-chloro-3,4-dihydroquinolin-2-one (140 mg, 0.488 mmol, 1 equiv) in toluene/DCM (10 mL, ½) was added 2-isocyanato-2-methylpropane (145.19 mg, 1.464 mmol, 3 equiv) dropwise at room temperature under nitrogen atmosphere. The final reaction mixture was stirred for 3 h at 90° C. Quenched with water (10 mL) and extracted with EA (3×10 mL). The combined extracts were washed with water, brine, dried over Na₂SO₄, filtered, and concentrated. The crude product was purified by Prep-HPLC to afford 1-(1-benzyl-7-chloro-2-oxo-3,4-dihydroquinolin-6-yl)-3-tert-butylurea (5.0 mg, 2.65%) as a white solid. MS (ESI): mass calcd. for C₂₁H₂₄ClN₃O₂: 385.16 m/z, found 386.15 [M+H]⁺. ¹H NMR (400 MHz, DMSO-d₆) δ 7.98 (s, 1H), 7.74 (s, 1H), 7.32-7.22 (m, 5H), 6.92 (s, 1H), 6.80 (s, 1H), 5.12 (s, 2H), 2.91 (t, J=7.2 Hz, 2H), 2.67 (t, J=7.2 Hz, 2H), 1.28 (s, 9H).

### Example 67: 1-(5-benzyl-6-oxo-5,6,7,8-tetrahydro-1,5-naphthyridin-2-yl)-3-(tert-butyl)urea

### Synthetic Scheme

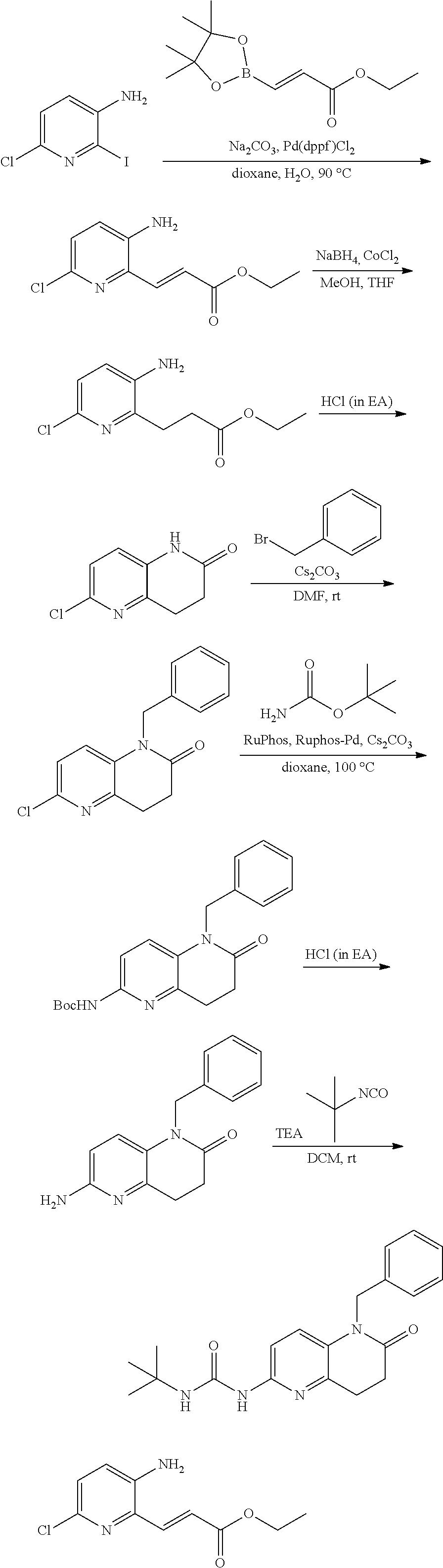

### ethyl (E)-3-(3-amino-6-chloropyridin-2-yl)acrylate

A solution of 6-chloro-2-iodopyridin-3-amine (2 g, 7.860 mmol, 1 equiv), ethyl (2E)-3-(4,4,5,5-tetramethyl-1,3,2-dioxaborolan-2-yl)prop-2-enoate (1.77 g, 7.860 mmol, 1 equiv), sodium methaneperoxoate sodium (2.5 g, 23.580 mmol, 3 equiv) and Pd(dppf)C₁₂ (575.11 mg, 0.786 mmol, 0.1 equiv) in water (3 mL) and 1,4-dioxane (30 mL) was stirred for overnight at 90° C. under N₂ atmosphere. The mixture was allowed to cool down to rt. The reaction progress was monitored by LCMS. The resulting mixture was extracted with EA (3×60 mL). The combined organic layers were washed with brine (1×100 mL). After filtration, the filtrate was concentrated under reduced pressure. The residue was purified by silica gel column chromatography (PE/EA=3:1) to afford ethyl (E)-3-(3-amino-6-chloropyridin-2-yl)acrylate (1.5 g, 84.20%) as a yellow solid. MS (ESI): mass calcd. for C₁₀H₁₁ClN₂O₂: 226.05 m/z, found 227.20 [M+H]⁺.

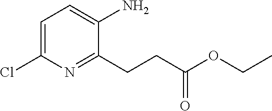

### ethyl 3-(3-amino-6-chloropyridin-2-yl)propanoate

A solution of ethyl (2E)-3-(3-amino-6-chloropyridin-2-yl)prop-2-enoate (1 g, 4.412 mmol, 1 equiv) in methanol (10 mL) and tetrahydrofuran (20 mL) was treated with cobalt (II) chloride (286.40 mg, 2.206 mmol, 0.5 equiv) for 30 min at 0° C. under nitrogen atmosphere followed by the addition of NaBH₄ (834.51 mg, 22.060 mmol, 5 equiv) in portions at 0° C. The resulting mixture was stirred for additional 1 h at rt. The reaction was quenched with H₂O at 0° C. The resulting mixture was filtered. The filter cake was washed with EA (50 mL). The filtrate was concentrated under reduced pressure. This resulted in ethyl 3-(3-amino-6-chloropyridin-2-yl)propanoate (950 mg, 94.16%) as a yellow solid. MS (ESI): mass calcd. for C₁₀H₁₃ClN₂O₂: 228.07 m/z, found 229.15 [M+H]+.

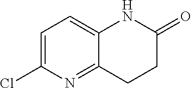 [SMILES: O=C1CCc2nc(Cl)ccc2N1]

### 6-chloro-3,4-dihydro-1,5-naphthyridin-2(1H)-one

To a stirred solution of ethyl 3-(3-amino-6-chloropyridin-2-yl)propanoate (900 mg, 3.936 mmol, 1 equiv) in HCl (2M, in EA) (30 mL) at rt and stirred for 2 h. The precipitated solids were collected by filtration and washed with diethyl ether (3×50 mL). This resulted in 6-chloro-3,4-dihydro-1H-1,5-naphthyridin-2-one (600 mg, 83.49%) as a white solid. MS (ESI): mass calcd. for C₈H₇ClN₂O: 182.02 m/z, found 183.05 [M+H]⁺.

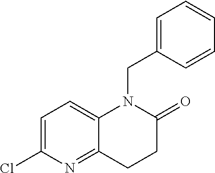 [SMILES: O=C1CCc2nc(Cl)ccc2N1Cc1ccccc1]

### 1-benzyl-6-chloro-3,4-dihydro-1,5-naphthyridin-2(1H)-one

To a stirred solution of 6-chloro-3,4-dihydro-1H-1,5-naphthyridin-2-one (600 mg, 3.286 mmol, 1 equiv) in anhydrous dimethylformamide (15 mL, 0.027 mmol, 0.17 equiv) was added benzyl bromide (730.57 mg, 4.272 mmol, 1.3 equiv) and Cs₂CO₃ (3.22 g, 9.858 mmol, 3 equiv) at rt and stirred for 2 h. After completion of reaction, the resulting mixture was extracted with EA (3×20 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA=(3:1) to afford 1-benzyl-6-chloro-3,4-dihydro-1,5-naphthyridin-2(1H)-one (680 mg, 75.88%) as a yellow solid. MS (ESI): mass calcd. for C₁₅H₁₃ClN₂O: 272.07 m/z, found 273.05 [M+H]⁺.

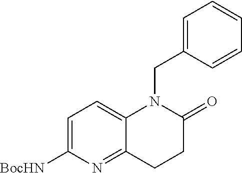 [SMILES: CC(C)(C)OC(=O)Nc1ccc2c(n1)CCC(=O)N2Cc1ccccc1]

### tert-butyl (1-benzyl-2-oxo-1,2,3,4-tetrahydro-1,7-naphthyridin-6-yl)carbamate

A solution of 1-benzyl-6-chloro-3,4-dihydro-1,5-naphthyridin-2-one (200 mg, 0.733 mmol, 1 equiv) in 1,4-dioxane (15 mL, 0.023 mmol, 0.31 equiv) was treated with tert-butyl carbamate (171.82 mg, 1.466 mmol, 2 equiv), Cs₂CO₃ (477.86 mg, 1.466 mmol, 2 equiv), EPHOS (39.22 mg, 0.073 mmol, 0.1 equiv) and EPhos Pd G4 (67.36 mg, 0.073 mmol, 0.1 equiv) for overnight at 90° C. under nitrogen atmosphere. The residue was purified by silica gel column chromatography, eluted with PE/EA (3:1) to afford tert-butyl (1-benzyl-2-oxo-1,2,3,4-tetrahydro-1,7-naphthyridin-6-yl)carbamate (180 mg, 69.45%) as a white solid. MS (ESI): mass calcd. for C₂₀H₂₃N₃O₃: 353.17 m/z, found 354.25 [M+H]⁺.

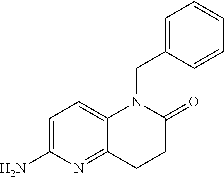 [SMILES: Nc1ccc2c(n1)CCC(=O)N2Cc1ccccc1]

### 6-amino-1-benzyl-3,4-dihydro-1,5-naphthyridin-2(1H)-one

Into a 20 mL bar were added tert-butyl N-(5-benzyl-6-oxo-7,8-dihydro-1,5-naphthyridin-2-yl)carbamate (80 mg, 0.226 mmol, 1 equiv) and HCl (10 mL, 10.000 mmol, 44.18 equiv) at rt and stirred for 3 h. The crude product/resulting mixture was used in the next step directly without further purification. This resulted in 6-amino-1-benzyl-3,4-dihydro-1,5-naphthyridin-2(1H)-one (60 mg, 75%) as a white solid. MS (ESI): mass calcd. for C₁₅H₁₅N₃O: 253.12 m/z, found 254.20 [M+H]⁺.

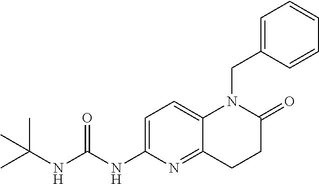 [SMILES: CC(C)(C)NC(=O)Nc1ccc2c(n1)CCC(=O)N2Cc1ccccc1]

### 1-(5-benzyl-6-oxo-5,6,7,8-tetrahydro-1,5-naphthyridin-2-yl)-3-(tert-butyl)urea

A solution of 6-amino-1-benzyl-3,4-dihydro-1,5-naphthyridin-2-one (60 mg, 0.237 mmol, 1 equiv), TEA (71.91 mg, 0.711 mmol, 3 equiv) and 2-isocyanato-2-methylpropane (117.41 mg, 1.185 mmol, 5 equiv) in DCM (10 mL, 157.306 mmol, 664.11 equiv) was stirred for overnight at rt. The residue was purified by silica gel column chromatography, eluted with EA (2:1) to afford 1-(5-benzyl-6-oxo-5,6,7,8-tetrahydro-1,5-naphthyridin-2-yl)-3-(tert-butyl)urea (43.1 mg, 51.44%) as a white solid. MS (ESI): mass calcd. for C₂₀H₂₄N₄O₂: 352.19 m/z, found 353.15 [M+H]+. ¹H NMR (300 MHz, DMSO-d₆) δ 8.87 (s, 1H), 7.95 (s, 1H), 7.37-7.16 (m, 6H), 7.11 (d, J=8.9 Hz, 1H), 5.10 (s, 2H), 2.99 (dd, J=8.7, 6.2 Hz, 2H), 2.80 (dd, J=8.8, 6.2 Hz, 2H), 1.31 (s, 9H).

### Example 68: 1-(1-benzyl-2-oxo-1,2-dihydroquinoxalin-6-yl)-3-(tert-butyl)urea

### Synthetic Scheme

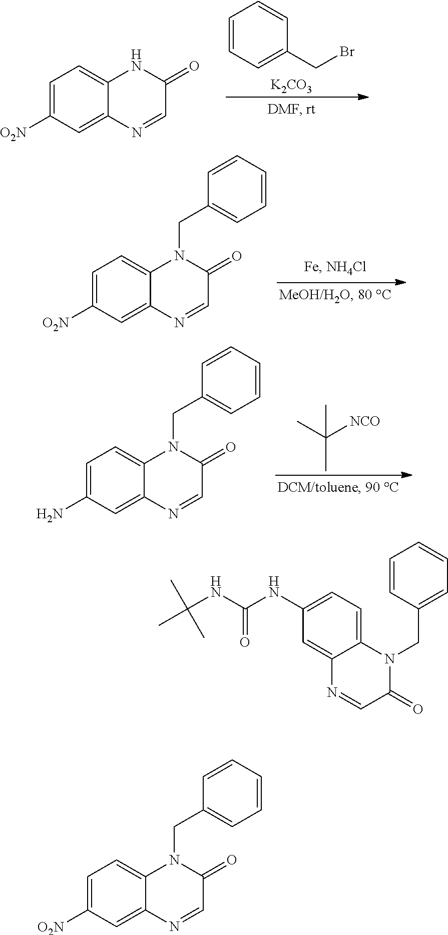

### 1-benzyl-6-nitroquinoxalin-2(1H)-one

To a solution of 6-nitro-1H-quinoxalin-2-one (500 mg, 2.616 mmol, 1 equiv) in DMF (20 mL) was added benzyl bromide (671.10 mg, 3.924 mmol, 1.5 equiv) and K₂CO₃ (728.31 mg, 5.232 mmol, 2 equiv). The reaction was stirred at rt for 1 hour. Quenched with water (10 mL) and extracted with EA (3×10 mL). The combined extracts were washed with water, brine, dried over Na₂SO₄, filtered, and concentrated. The residue was purified by column chromatography on silica gel with EA/PE (0-100%) to give 1-benzyl-6-nitroquinoxalin-2-one (450 mg, 61.16%) as a yellow solid. MS (ESI): mass calcd. for C₁₅H₁₁N₃O₃: 281.27m/z, found 282.00 [M+H]⁺.

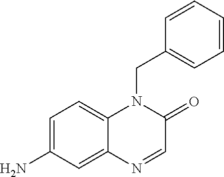 [SMILES: Nc1ccc2c(c1)ncc(=O)n2Cc1ccccc1]

### 6-amino-1-benzylquinoxalin-2(1H)-one

To a solution of 1-benzyl-6-nitroquinoxalin-2-one (250 mg, 0.889 mmol, 1 equiv) in MeOH/H₂O (11 mL, 10:1) was added Fe (496.36 mg, 8.890 mmol, 10 equiv) and NH₄Cl (475.43 mg, 8.890 mmol, 10 equiv). The reaction was stirred at 80° C. for 1 hour. The reaction was quenched with water at rt. After filtration, the filter cake was washed with EA (3×20 mL). The filtrate was added water (20 mL) and extracted with EA (3×20 mL). The combined extracts were washed with water, brine, dried over Na₂SO₄, filtered, and concentrated. This resulted in 6-amino-1-benzylquinoxalin-2-one (220 mg, 98.50%) as a yellow solid. MS (ESI): mass calcd. for C₁₅H₁₃N₃O: 251.29 m/z, found 252.05 [M+H]⁺.

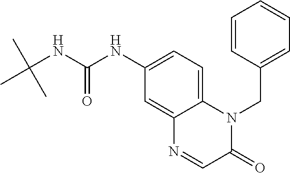 [SMILES: CC(C)(C)NC(=O)Nc1ccc2c(c1)ncc(=O)n2Cc1ccccc1]

### 1-(1-benzyl-2-oxo-1,2-dihydroquinoxalin-6-yl)-3-(tert-butyl)urea

To a solution of 6-amino-1-benzylquinoxalin-2-one (220 mg, 0.875 mmol, 1 equiv) in DCM/Toluene (15 mL, 2:1) was added 2-isocyanato-2-methylpropane (520.74 mg, 5.250 mmol, 6 equiv). The reaction was stirred at 90° C. for 2 hours. Quenched with water (20 mL) and extracted with EA (3×20 mL). The combined extracts were washed with water, brine, dried over Na₂SO₄, filtered, and concentrated. The residue was purified by preparative HPLC using a XBridge Prep OBD C18 Column, 30*150 mm, 5 μm column (eluent: 26% to 45% (v/v) CH₃CN and H₂O with 10 mmol/L NH₄HCO₃) to afford the title compound 1-(1-benzyl-2-oxoquinoxalin-6-yl)-3-tert-butylurea (55 mg, 17.93%) as a yellow solid. MS (ESI): mass calcd. for C₂₀H₂₂N₄O₂: 350.42 m/z, found:351.20 [M+H]⁺. ¹H NMR (400 MHz, DMSO-d₆) δ 8.40-8.52 (m, 1H), 8.25-7.38 (m, 1H), 7.83-8.01 (m, 1H), 7.20-7.50 (m, 7H), 5.90-6.10 (m, 1H), 5.35-5.61 (m, 2H), 1.19-1.39 (m, 9H).

### Example 69: 1-(1-benzyl-8-fluoro-2-oxo-1,2,3,4-tetrahydroquinolin-6-yl)-3-(tert-butyl)urea

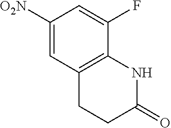 [SMILES: O=C1CCc2cc([N+](=O)[O-])cc(F)c2N1]

### 8-fluoro-6-nitro-3,4-dihydroquinolin-2(1H)-one

To a solution of 8-fluoro-3,4-dihydro-1H-quinolin-2-one (500 mg, 3.027 mmol, 1 equiv) in H₂SO₄ (3 mL) was added HNO₃ (190.7 mg, 3.027 mmol, 1.0 equiv) at −10° C. The resulting mixture was stirred at −10° C. until the starting material was totally consumed by LCMS, The reaction mixture was quenched by water and adjusting PH with saturated sodium bicarbonate aqueous solution and then extracted with EA (3×20 mL). The combined organic extracts were washed with brine (50 mL), dried over anhydrous Na₂SO₄, and concentrated under vacuum to yield a crude product which was directly purified by flash chromatography (PE/EA=3:1) to afford 8-fluoro-6-nitro-3,4-dihydro-1H-quinolin-2-one (300 mg, 46.66%) as a yellow solid. MS (ESI): mass calcd. for C₉H₇FN₂O₃, 210.04 m/z, found 211.10 [M+H]⁺

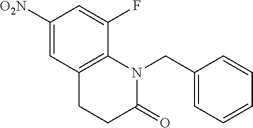 [SMILES: O=C1CCc2cc([N+](=O)[O-])cc(F)c2N1Cc1ccccc1]

### 1-benzyl-8-fluoro-6-nitro-3,4-dihydroquinolin-2(1H)-one

To a solution of 8-fluoro-6-nitro-3,4-dihydro-1H-quinolin-2-one (250 mg, 1.190 mmol, 1 equiv) in DMF (10 mL) was added K₂CO₃ (493.2 mg, 3.569 mmol, 3 equiv) and benzyl bromide (305.1 mg, 1.784 mmol, 1.5 equiv). The resulting mixture was placed at rt and stirred overnight until the starting material was totally consumed by LCMS. The mixture was diluted with EtOAc (100 mL), washed with water (3×30 mL), brine (2×30 mL) and concentrated under vacuum to yield a crude product which was directly purified by flash chromatography (0-100% ethyl acetate/petroleum ether) to afford 4-benzyl-7-nitro-2H-1,4-benzoxazin-3-one (250 mg, 65.39%) as a yellow solid. MS (ESI): mass calcd. for C₁₆H₁₃FN₂O₃: 300.09 m/z, found 301.15 [M+H]+.

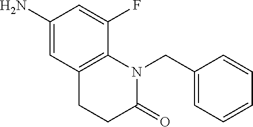 [SMILES: Nc1cc(F)c2c(c1)CCC(=O)N2Cc1ccccc1]

### 6-amino-1-benzyl-8-fluoro-3,4-dihydroquinolin-2(1H)-one

To a solution of 1-benzyl-8-fluoro-6-nitro-3,4-dihydroquinolin-2-one (250 mg, 0.833 mmol, 1 equiv) in MeOH (10 mL) was added Pd/C (30 mg). The reaction mixture was then stirred for 1 h under hydrogen atmosphere (balloon). The reaction mixture was filtered through a pad of Celite and concentrated under reduced pressure to afford 6-amino-1-benzyl-8-fluoro-3,4-dihydroquinolin-2-one (180 mg, 77.83%) as a yellow solid. MS (ESI): mass calcd. for C₁₆H₁₅FN₂O, 270.12 m/z, found 271.10 [M+H]⁺.

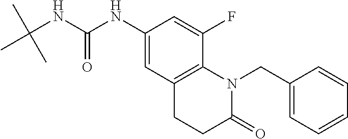 [SMILES: CC(C)(C)NC(=O)Nc1cc(F)c2c(c1)CCC(=O)N2Cc1ccccc1]

### 1-(1-benzyl-8-fluoro-2-oxo-1,2,3,4-tetrahydroquinolin-6-yl)-3-(tert-butyl)urea

To a stirred solution of 6-amino-1-benzyl-8-fluoro-3,4-dihydroquinolin-2-one (140 mg, 0.518 mmol, 1 equiv) in DCM (10.00 mL) was added 2-isocyanato-2-methylpropane (256.7 mg, 2.590 mmol, 5 equiv) and TEA (157.2 mg, 1.554 mmol, 3 equiv) stirred overnight at rt. The reaction progress was monitored by LCMS. The reaction mixture was quenched by water and extracted with EA (3*20 mL). The combined organic extracts were washed with brine (20 mL), dried over anhydrous Na₂SO₄, and concentrated under vacuum to yield a crude product which was directly purified by flash chromatography (0-100% ethyl acetate/petroleum ether) to afford 100 mg crude product as a white solid. The compound was separated by preparative HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 39% B to 58% B in 7 min; Wave Length: 254 nm; RT1(min): 6.4; Number Of Runs: 3) to afford 1-(1-benzyl-8-fluoro-2-oxo-3,4-dihydroquinolin-6-yl)-3-tert-butylurea (23.4 mg, 12.13%) as a white solid. MS (ESI): mass calcd. for C₂₁H₂₄FN₃O₂, 369.19 m/z, found 370.15 [M+H]⁺. ¹H NMR (300 MHz, DMSO-d₆) δ 8.31 (s, 1H), 7.26 (t, J=7.3 Hz, 2H), 7.08-7.21 (m, 4H), 6.87 (s, 1H), 6.01 (s, 1H), 5.11 (s, 2H), 2.86 (t, J=6.7 Hz, 2H), 2.54-2.70 (m, 2H), 1.25 (s, 9H).

### Example 70: 1-(tert-butyl)-3-(3-methyl-2-oxo-1-(3-(trifluoromethyl)benzyl)-1,2,3,4-tetrahydroquinazolin-6-yl)urea

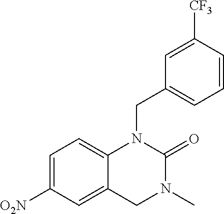 [SMILES: CN1Cc2cc([N+](=O)[O-])ccc2N(Cc2cccc(C(F)(F)F)c2)C1=O]

### 3-methyl-6-nitro-1-(3-(trifluoromethyl)benzyl)-3,4-dihydroquinazolin-2(1H)-one

To a solution of 3-methyl-6-nitro-1,4-dihydroquinazolin-2-one (120 mg, 0.579 mmol, 1 equiv) in DMF (10 mL) was added 1-(bromomethyl)-3-(trifluoromethyl)benzene (152.29 mg, 0.637 mmol, 1.1 equiv) and Cs₂CO₃ (378.58 mg, 1.158 mmol, 2 equiv). The reaction was stirred at rt for 1 hour. Quenched with water (50 mL) and extracted with EA (3×50 mL). The combined extracts were washed with water, brine, dried over Na₂SO₄, filtered, and concentrated. The residue was purified by column chromatography on silica gel with EA/PE (0-100%) to give 3-methyl-6-nitro-1-{[3-(trifluoromethyl)phenyl]methyl}-4H-quinazolin-2-one (170 mg, 80.35%) as a yellow solid. MS (ESI): mass calcd. for C₁₇H₁₄F₃N₃O₃: 365.31m/z, found 366.05 [M+H]⁺.

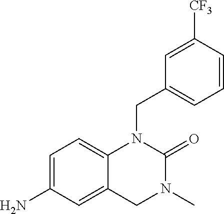 [SMILES: CN1Cc2cc(N)ccc2N(Cc2cccc(C(F)(F)F)c2)C1=O]

### 6-amino-3-methyl-1-(3-(trifluoromethyl)benzyl)-3,4-dihydroquinazolin-2(1H)-one

To a solution of 3-methyl-6-nitro-1-{[3-(trifluoromethyl)phenyl]methyl}-4H-quinazolin-2-one (170 mg, 0.465 mmol, 1 equiv) in MeOH/H₂O (11 mL, 10:1) was added Fe (259.88 mg, 4.650 mmol, 10 equiv) and NH₄Cl (248.92 mg, 4.650 mmol, 10 equiv). The reaction was stirred for 1 hour at 80° C. The reaction was quenched with water at rt. After filtration, the filter cake was washed with EA (3×20 mL). The filtrate was added water (20 mL) and extracted with EA (3×20 mL). The combined extracts were washed with water, brine, dried over Na₂SO₄, filtered, and concentrated. This resulted in 6-amino-3-methyl-1-{[3-(trifluoromethyl)phenyl]methyl}-4H-quinazolin-2-one (150 mg, 96.15%) as a yellow oil. MS (ESI): mass calcd. for C₁₇H₁₆F₃N₃O: 335.33m/z, found 336.05 [M+H]⁺.

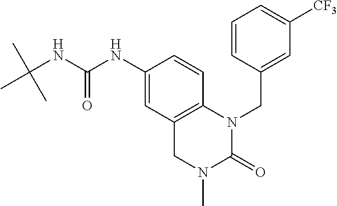 [SMILES: CN1Cc2cc(NC(=O)NC(C)(C)C)ccc2N(Cc2cccc(C(F)(F)F)c2)C1=O]

### 1-(tert-butyl)-3-(3-methyl-2-oxo-1-(3-(trifluoromethyl)benzyl)-1,2,3,4-tetrahydroquinazolin-6-yl)urea

To a solution of 6-amino-3-methyl-1-{[3-(trifluoromethyl)phenyl]methyl}-4H-quinazolin-2-one (160 mg, 0.477 mmol, 1 equiv) in DCM/Toluene (15 mL, 2:1) was added 2-isocyanato-2-methylpropane (283.80 mg, 2.862 mmol, 6 equiv). The reaction was stirred at 90° C. for 2 hours. Quenched with water (50 mL) and extracted with EA (3×50 mL). The combined extracts were washed with water, brine, dried over Na₂SO₄, filtered, and concentrated. The residue was purified by preparative HPLC using a XBridge Prep OBD C18 Column, 30*150 mm, 5 μm column (eluent: 17% to 42% (v/v) CH₃CN and H₂O with 10 mmol/L NH₄HCO₃) to afford the title compound 3-tert-butyl-1-(3-methyl-2-oxo-1-{[3-(trifluoromethyl)phenyl]methyl}-4H-quinazolin-6-yl)urea (21.3 mg, 10.27%) as a white solid. MS (ESI): mass calcd. for C₂₂H₂₅F₃N₄O₂: 434.46 m/z, found:435.20 [M+H]⁺. ¹H NMR (400 MHz, DMSO-d₆) δ 7.91-8.20 (m, 1H), 7.43-7.66 (m, 4H), 7.17-7.36 (m, 1H), 6.84-7.02 (m, 1H), 6.45-6.62 (m, 1H), 5.76-6.00 (m, 1H), 4.94-5.20 (m, 2H), 4.30-4.48 (m, 2H), 2.85-3.01 (m, 3H), 1.17-1.33 (m, 9H). ¹⁹F NMR (376 MHz, DMSO) δ (ppm): −60.9887.

### Experimental Procedures

### Example 71: 1-(tert-butyl)-3-(4-(3-(difluoromethyl)benzyl)-3-oxo-3,4-dihydro-2H-benzo[b][1,4]oxazin-7-yl)urea

### Synthetic Scheme

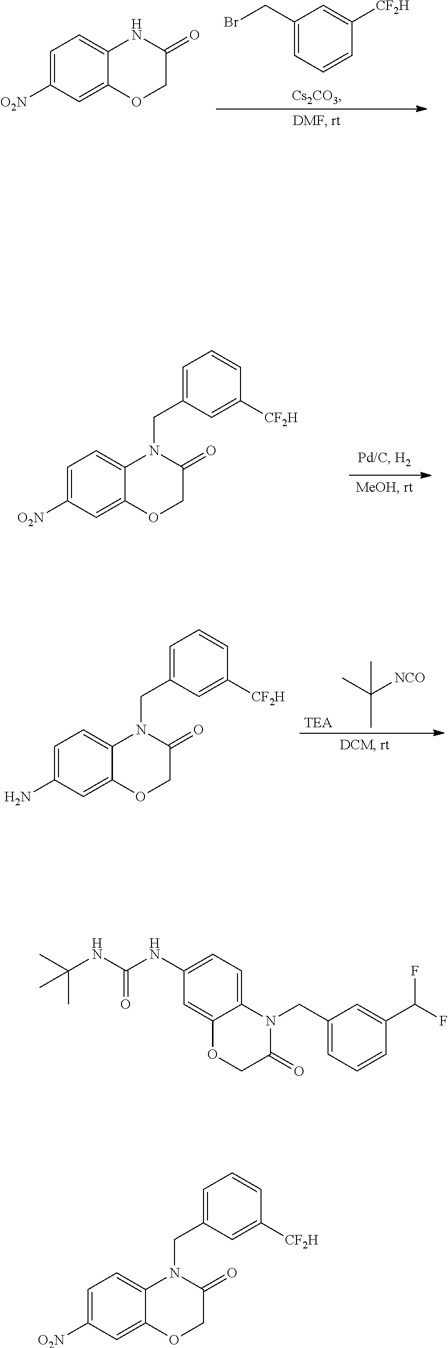

### 4-(3-(difluoromethyl)benzyl)-7-nitro-2H-benzo[b][1,4]oxazin-3(4H)-one

To a stirred solution of 7-nitro-2H-benzo[b][1,4]oxazin-3(4H)-one (300 mg, 1.545 mmol, 1 equiv) in DMF (15 mL) was added 1-(bromomethyl)-3-(difluoromethyl)benzene (376 mg, 1.700 mmol, 1.1 equiv) and Cs₂CO₃ (1.5 g, 4.635 mmol, 3 equiv) at rt and stirred for overnight. The reaction was quenched with water (10 mL). The aqueous layer was extracted with EtOAc (3×10 mL). The residue was purified by silica gel column chromatography, eluted with PE/EA (3:1) to afford 4-(3-(difluoromethyl)benzyl)-7-nitro-2H-benzo[b][1,4]oxazin-3(4H)-one (220 mg, 42.59%).

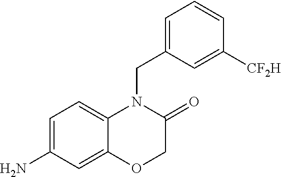 [SMILES: Nc1ccc2c(c1)OCC(=O)N2Cc1cccc(C(F)F)c1]

### 7-amino-4-(3-(difluoromethyl)benzyl)-2H-benzo[b][1,4]oxazin-3(4H)-one

To a solution of 4-(3-(difluoromethyl)benzyl)-7-nitro-2H-benzo[b][1,4]oxazin-3(4H)-one (100 mg, 0.299 mmol, 1 equiv) in MeOH (15 mL) was added Pd/C (50 mg) under nitrogen atmosphere. The resulting mixture was placed at rt under a hydrogen atmosphere until the starting material was totally consumed by LCMS. The mixture was filtered and concentrated under vacuum to afford The catalyst was removed by filtration through a pad of Celite and rinsed with MeOH. The filtrate was concentrated to afford 7-amino-4-(3-(difluoromethyl)benzyl)-2H-benzo[b][1,4]oxazin-3(4H)-on (57 mg, 62.62%) as a white solid.

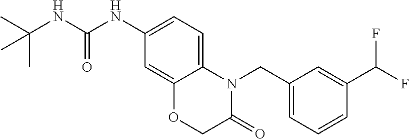 [SMILES: CC(C)(C)NC(=O)Nc1ccc2c(c1)OCC(=O)N2Cc1cccc(C(F)F)c1]

### 1-(tert-butyl)-3-(4-(3-(difluoromethyl)benzyl)-3-oxo-3,4-dihydro-2H-benzo[b][1,4]oxazin-7-yl)urea

A solution of 7-amino-4-(3-(difluoromethyl)benzyl)-2H-benzo[b][1,4]oxazin-3(4H)-one (50 mg, 0.164 mmol, 1 equiv), TEA (49.88 mg) and 2-isocyanato-2-methylpropane (48.87 mg, 0.492 mmol, 3 equiv) in DCM (5 mL) was stirred for overnight at rt. The residue was purified by silica gel column chromatography, eluted with EA (35%-46%) to afford crude product. The residue was purified by Prep-HPLC with the following conditions (Column: YMC-Actus Triart C18, 30*150 mm, 5 μm; Mobile Phase A: Water (10 mmol/L NH₄HCO₃), Mobile Phase B: ACN; Flow rate: 60 mL/min; Gradient: 45% B to 61% B in 9 min, 61% B; Wave Length: 220/254 nm) to afford 1-(tert-butyl)-3-(4-(3-(difluoromethyl)benzyl)-3-oxo-3,4-dihydro-2H-benzo[b][1,4]oxazin-7-yl)urea (4 mg, 6.03%) as a white solid. MS (ESI): mass calcd. for C₂₁H₂₃F₂N₃O₃: 403.17 m/z, found 404.15 [M+H]⁺. ¹H NMR (300 MHz, DMSO-d₆) δ 8.22 (s, 1H), 7.44-7.51 (m, 4H), 7.21 (s, 1H), 7.01 (s, 1H), 6.82-6.88 (m, 1H), 6.76 (d, J=9.0 Hz, 1H), 5.93 (s, 1H), 5.16 (s, 2H), 4.75 (s, 2H), 1.25 (s, 9H).

The following examples in Table 3 were made according to the procedure for Example 71.

TABLE 3

| Structure | Ex. | IUPAC Name | 1H NMR | LCMS |
|---|---|---|---|---|
| 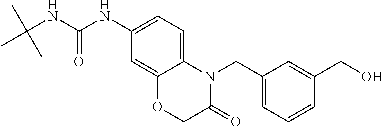 [SMILES: CC(C)(C)NC(=O)Nc1ccc2c(c1)OCC(=O)N2Cc1cccc(CO)c1] | 72 | 1-(tert-butyl)-3-(4- (3-(hydroxy- methyl)ben- zyl)-3-oxo-3,4- dihydro-2H- benzo[b][1,4]oxa- zin-7-yl)urea | ¹H NMR (300 MHz, DMSO-d₆) δ 8.22 (s, 1H), 7.08-7.31 (m, 5H), 6.85 (d, J = 8.8 Hz, 1H), 6.71- 6.78 (m, 1H), 5.94 (s, 1H), 5.18 (t, J = 5.7 Hz, 1H), 5.09 (s, 2H), 4.73 (s, 2H), 4.45 (d, J = 5.6 Hz, | calcd. for C₂₁H₂₅N₃O₄, 383.18 m/z, found 384.05 [M + H]⁺ |
| | | | 2H), 1.25 (s, 9H). | |
| 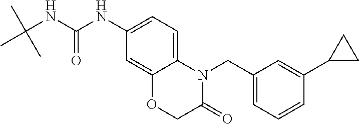 [SMILES: CC(C)(C)NC(=O)Nc1ccc2c(c1)OCC(=O)N2Cc1cccc(C2CC2)c1] | 73 | 1-(tert-butyl)-3-(4- (3- cyclopropylbenzyl)- 3-oxo-3,4- dihydro-2H- benzo[b][1,4]oxa- zin-7-yl)urea | ¹H NMR (400 MHz, DMSO-d₆) δ 8.21 (s, 1H), 7.14-7.24(m, 2H), 6.81- 7.07 (m, 4H), 6.72-6.79 (m, 1H), 5.93 (s, 1H), 5.05 (s, 2H), 4.74 (s, 2H), 1.87 (tt, J = 8.4, 5.1 Hz, 1H), 1.26 (s, 9H), 0.91- | calcd. for C₂₃H₂₇N₃O₃: 393.21 m/z, found 394.20 [M + H]⁺ |
| | | | 0.97 (m, 2H), 0.59-0.68 | |
| | | | (m, 2H). | |
| 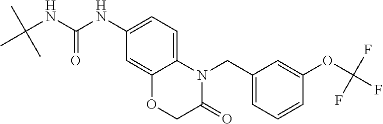 [SMILES: CC(C)(C)NC(=O)Nc1ccc2c(c1)OCC(=O)N2Cc1cccc(OC(F)(F)F)c1] | 74 | 1-(tert-butyl)-3-(3- oxo-4-(3-(tri- fluoromethoxy)ben- zyl)-3,4-dihydro- 2H- benzo[b][1,4]oxa- zin-7-yl)urea | ¹H NMR (300 MHz, DMSO-d₆) δ 8.22 (s, 1H), 7.47 (t, J = 8.1 Hz, 1H), 7.33-7.23 (m, 3H), 7.21 (d, J = 2.3 Hz, 1H), 6.87 (d, J = 8.8 Hz, 1H), 6.78 (dd, J = 8.8, 2.3 Hz, 1H), 5.93 (s, 1H), 5.16 (s, 2H), | calcd. for C₂₁H₂₂F₃N₃O₄, 437.16 m/z, found 438.15 [M + H]⁺ |
| | | | 4.76 (s, 2H), 1.27 (s, 9H). | |
| 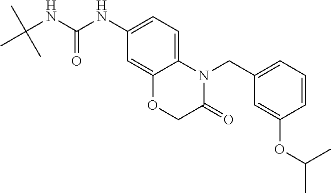 [SMILES: CC(C)Oc1cccc(CN2C(=O)COc3cc(NC(=O)NC(C)(C)C)ccc32)c1] | 75 | 1-(tert-butyl)-3-(4- (3- isopropoxybenzyl)- 3-oxo-3,4-dihydro- 2H- benzo[b][1,4]oxa- zin-7-yl)urea | ¹H NMR (300 MHz, DMSO-d₆) δ 8.21 (s, 1H), 7.21 (d, J = 2.5 Hz, 2H), 6.88 (d, J = 8.8 Hz, 1H), 6.82-6.71 (m, 4H), 5.93 (s, 1H), 5.06 (s, 2H), 4.74 (s, 2H), 4.56 (p, J = 6.0 Hz, 1H), 1.26 (s, 9H), 1.24 (d, J = 6.0 Hz, 6H). | calcd. for C₂₃H₂₉N₃O₄: 411.22 m/z, found 412.25 [M + H]⁺ |
| 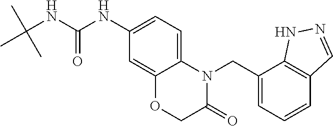 [SMILES: CC(C)(C)NC(=O)Nc1ccc2c(c1)OCC(=O)N2Cc1cccc2cn[nH]c12] | 76 | 1-(4-((1H-indazol- 7-y1)methyl)-3- oxo-3,4-dihydro- 2H- benzo[b][1,4]oxa- zin-7-yl)-3-(tert- butyl)urea | ¹H NMR (400 MHz, DMSO- d₆) δ 13.30 (s, 1H), 8.22 (s, 1H), 8.16 (s, 1H), 7.67 (d, J = 8.0 Hz, 1H), 7.27 (d, J = 2.2 Hz, 1H), 7.03 (t, J = 7.5 Hz, 1H), 6.95 (d, J = 7.1 Hz, 1H), | calcd. for C₂₁H₂₃N₅O₃, 393.18 m/z, found 394.15 [M + H]⁺ |
| | | | 6.68-6.78 (m, 2H), 5.93 | |
| | | | (s, 1H), 5.37 (s, 2H), | |
| | | | 4.81 (s, 2H), 1.26 (s, 9H). | |
| 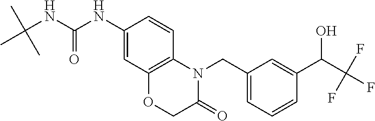 [SMILES: CC(C)(C)NC(=O)Nc1ccc2c(c1)OCC(=O)N2Cc1cccc(C(O)C(F)(F)F)c1] | 77 | 1-(tert-butyl)-3-(3- oxo-4-(3-(2,2,2- trifluoro-1-hy- droxyethyl)benzyl)- 3,4-dihydro- 2H- benzo[b][1,4]oxa- zin-7-yl)urea | ¹H NMR (300 MHz, DMSO-d₆) δ 8.21 (s, 1H), 7.42 (s, 1H), 7.36 (d, J = 4.7 Hz, 2H), 7.23-7.27 (m, 1H), 7.19 (d, J = 2.2 Hz, 1H), 6.80-6.90 (m, 2H), 6.75 (dd, J = 8.7, 2.3 Hz, 1H), 5.92 (s, 1H), | calcd. for C₂₂H₂₄F₃N₃O₄, 451.17 m/z, found 452.10 [M + H]⁺ |
| | | | 5.12 (s, 2H), 5.11 (s, 1H), | |
| | | | 4.73 (s, 2H), 1.25 (s, 9H). | |
| 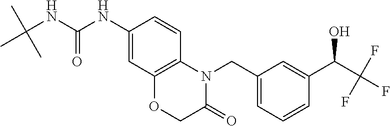 [SMILES: CC(C)(C)NC(=O)Nc1ccc2c(c1)OCC(=O)N2Cc1cccc([C@@H](O)C(F)(F)F)c1] | 78 | (R)-1-(tert-butyl)- 3-(3-oxo-4-(3- (2,2,2-trifluoro-1- hydroxyethyl)ben- zyl)-3,4-dihydro- 2H- benzo[b][1,4]oxa- zin-7-yl)urea | ¹H NMR (400 MHz, DMSO-d₆) δ 8.20 (s, 1H), 7.42 (s, 1H), 7.31-7.39 (m, 2H), 7.22-7.29 (m, 1H), 7.18 (d, J = 2.3 Hz, 1H), 6.80-6.88 (m, 2H), 6.75 (dd, J = 8.8, 2.4 Hz, 1H), 5.92 (s, 1H), 5.10- | calcd. for C₂₂H₂₄F₃N₃O₄, 451.17 m/z, found 452.10 [M + H]⁺ |
| | | | 5.14 (m, 3H), 4.73 (s, | |
| | | | 2H), 1.25 (s, 9H). | |
| 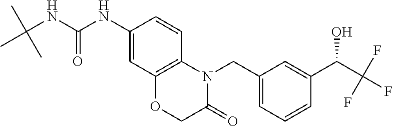 [SMILES: CC(C)(C)NC(=O)Nc1ccc2c(c1)OCC(=O)N2Cc1cccc([C@H](O)C(F)(F)F)c1] | 79 | (S)-1-(tert-butyl)-3- (3-oxo-4-(3-(2,2,2- trifluoro-1-hy- droxyethyl)benzyl)- 3,4-dihydro- 2H- benzo[b][1,4]oxa- zin-7-yl)urea | ¹H NMR (400 MHz, DMSO-d₆) δ 8.20 (s, 1H), 7.42 (s, 1H), 7.31-7.39 (m, 2H), 7.22-7.29 (m, 1H), 7.18 (d, J = 2.3 Hz, 1H), 6.80-6.88 (m, 2H), 6.75 (dd, J = 8.8, 2.4 Hz, 1H), 5.92 (s, 1H), 5.10- | calcd. for C₂₂H₂₄F₃N₃O₄, 451.17 m/z, found 452.10 [M + H]⁺ |
| | | | 5.14 (m, 3H), 4.73 (s, | |
| | | | 2H), 1.25 (s, 9H). | |
| 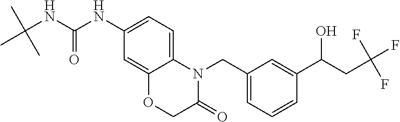 [SMILES: CC(C)(C)NC(=O)Nc1ccc2c(c1)OCC(=O)N2Cc1cccc(C(O)CC(F)(F)F)c1] | 80 | 1-(tert-butyl)-3-(3- oxo-4-(3-(3,3,3- trifluoro-1- hydroxypropyl)ben- zyl)-3,4-dihydro- 2H- benzo[b][1,4]oxa- zin-7-yl)urea | ¹H NMR (300 MHz, DMSO-d₆) δ 8.20 (s, 1H), 7.35 (s, 1H), 7.32-7.28 (m, 2H), 7.18-7.12 (m, 2H), 6.85 (d, J = 8.7 Hz, 1H), 6.77-6.73 (m, 1H), 5.92 (s, 1H), 5.67 (d, J = 5.1 Hz, 1H), 5.09 (s, 2H), | calcd. for C₂₃H₂₆F₃N₃O₄: 465.2 m/z, found 466.10 [M + H]⁺ |
| | | | 4.86 (q, J = 5.1 Hz, 1H), | |
| | | | 4.74 (s, 2H), 2.50 (q, J = | |
| | | | 1.8 Hz, 2H), 1.26 (s, 9H). | |
| | | | ¹⁹F NMR (282 MHz, | |
| | | | DMSO-d₆) δ −61.56. | |

### Examples 81 and 82: (S)-1-(tert-butyl)-3-(2-methyl-3-oxo-4-(3-(trifluoromethyl)benzyl)-3,4-dihydro-2H-benzo[b][1,4]oxazin-7-yl)urea and (R)-1-(tert-butyl)-3-(2-methyl-3-oxo-4-(3-(trifluoromethyl)benzyl)-3,4-dihydro-2H-benzo[b][1,4]oxazin-7-yl)urea

### Synthetic Scheme

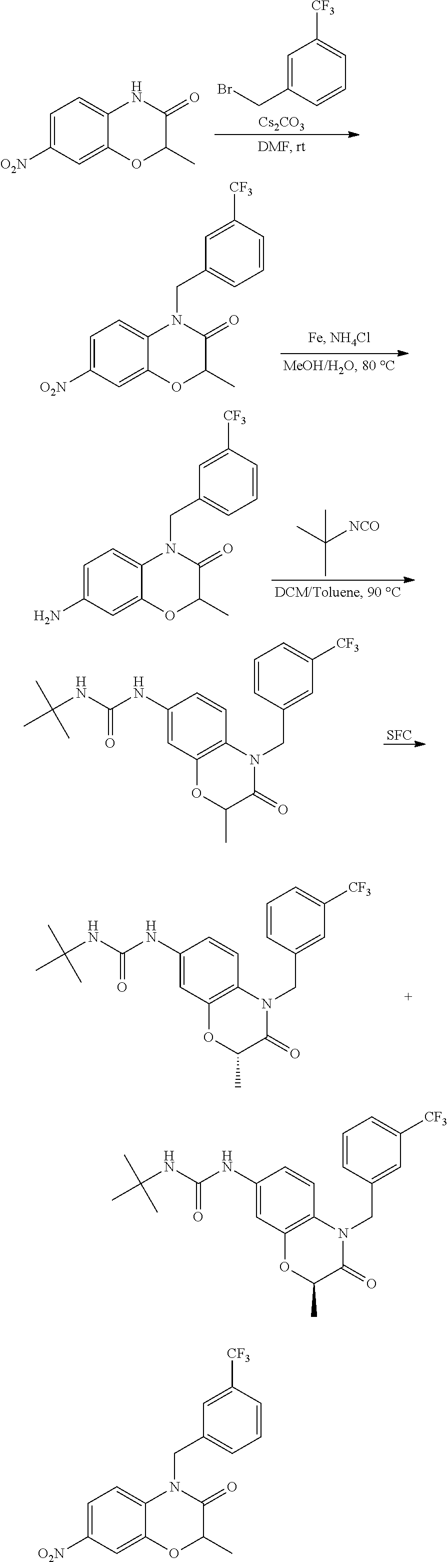

### 2-methyl-7-nitro-4-(3-(trifluoromethyl)benzyl)-2H-benzo[b][1,4]oxazin-3(4H)-one

To a solution of 2-methyl-7-nitro-2,4-dihydro-1,4-benzoxazin-3-one (250 mg, 1.201 mmol, 1 equiv) in DMF (10 mL) was added 1-(bromomethyl)-3-(trifluoromethyl)benzene (315.77 mg, 1.321 mmol, 1.1 equiv) and Cs₂CO₃ (784.99 mg, 2.402 mmol, 2 equiv). The reaction was stirred at rt for 1 hours. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 2-methyl-7-nitro-4-{[3-(trifluoromethyl)phenyl]methyl}-2H-1,4-benzoxazin-3-one (390 mg, 88.660%) as a yellow solid. MS (ESI): mass calcd. for C₁₇H₁₃F₃N₂O₄: 366.08 m/z, found 367.00 [M+H]⁺.

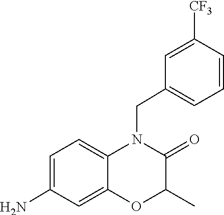 [SMILES: CC1Oc2cc(N)ccc2N(Cc2cccc(C(F)(F)F)c2)C1=O]

### 7-amino-2-methyl-4-(3-(trifluoromethyl)benzyl)-2H-benzo[b][1,4]oxazin-3(4H)-one

To a solution of 2-methyl-7-nitro-4-{[3-(trifluoromethyl)phenyl]methyl}-2H-1,4-benzoxazin-3-one (390 mg, 1.065 mmol, 1 equiv) in MeOH/H₂O (10:1, 11 mL) was added Fe (594.59 mg, 10.650 mmol, 10 equiv) and NH₄Cl (569.51 mg, 10.650 mmol, 10 equiv). The resulting mixture was filtered, and the filter cake was washed with EA (3×30 mL). The filtrate was concentrated under reduced pressure. The mixture was diluted with water (30 mL) and extracted with EA (3×30 mL). The combined extracts were washed with water, brine, dried over Na₂SO₄, filtered and concentrated. This resulted in 7-amino-2-methyl-4-{[3-(trifluoromethyl)phenyl]methyl}-2H-1,4-benzoxazin-3-one (350 mg, 97.74%) as a yellow oil. MS (ESI): mass calcd. for C₁₇H₁₅F₃N₂O₂: 336.11m/z, found 337.05 [M+H]⁺.

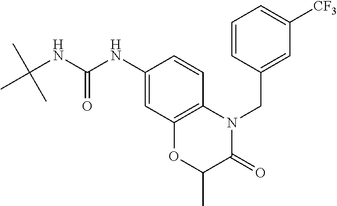 [SMILES: CC1Oc2cc(NC(=O)NC(C)(C)C)ccc2N(Cc2cccc(C(F)(F)F)c2)C1=O]

### 1-(tert-butyl)-3-(2-methyl-3-oxo-4-(3-(trifluoromethyl)benzyl)-3,4-dihydro-2H-benzo[b][1,4]oxazin-7-yl)urea

To a solution of 7-amino-2-methyl-4-{[3-(trifluoromethyl)phenyl]methyl}-2H-1,4-benzoxazin-3-one (190 mg, 0.565 mmol, 1 equiv) in DCM/Toluene (2:1, 15 mL) was added 2-isocyanato-2-methylpropane (336.03 mg, 3.390 mmol, 6 equiv). The reaction was stirred at 90° C. for 2 hours. After cooling down to rt, the reaction was quenched with water (50 mL) and extracted with EA (3×50 mL). The combined extracts were washed with water, brine, dried over Na₂SO₄, filtered and concentrated. The residue was purified by preparative HPLC using a XBridge Prep OBD C18 Column, 30*150 mm, 5 μm column (eluent: 26% to 42% (v/v) CH₃CN and H₂O with 10 mmol/L NH₄HCO₃) to afford the title compound 3-tert-butyl-1-(2-methyl-3-oxo-4-{[3-(trifluoromethyl)phenyl]methyl}-2H-1,4-benzoxazin-7-yl)urea (40 mg, 16.25%) as a white solid. MS (ESI): mass calcd. for C₂₂H₂₄F₃N₃O₃: 435.18 m/z, found:436.20 [M+H]⁺.

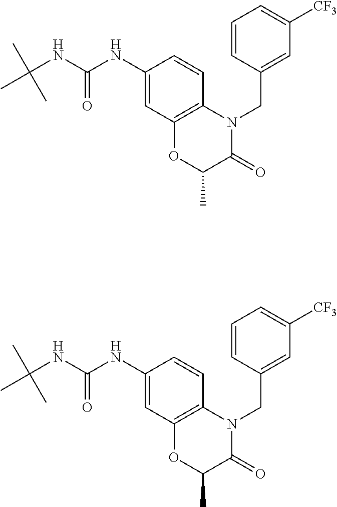 [SMILES: C[C@@H]1Oc2cc(NC(=O)NC(C)(C)C)ccc2N(Cc2cccc(C(F)(F)F)c2)C1=O ; C[C@H]1Oc2cc(NC(=O)NC(C)(C)C)ccc2N(Cc2cccc(C(F)(F)F)c2)C1=O]

### (S)-1-(tert-butyl)-3-(2-methyl-3-oxo-4-(3-(trifluoromethyl)benzyl)-3,4-dihydro-2H-benzo[b][1,4]oxazin-7-yl)urea and (R)-1-(tert-butyl)-3-(2-methyl-3-oxo-4-(3-(trifluoromethyl)benzyl)-3,4-dihydro-2H-benzo[b][1,4]oxazin-7-yl)urea

A sample of 3-tert-butyl-1-(2-methyl-3-oxo-4-{[3-(trifluoromethyl)phenyl]methyl}-2H-1,4-benzoxazin-7-yl)urea (40 mg) was separated by chiral-HPLC using a CHIRALPAK ID, 2*25 cm, 5 μm column (eluent: 10% to 10% (v/v) Hex(0.5% 2M NH₃-MeOH) and EtOH) to yield first enantiomer example 3-tert-butyl-1-[(2R)-2-methyl-3-oxo-4-{[3-(trifluoromethyl)phenyl]methyl}-2H-1,4-benzoxazin-7-yl]urea (16.4 mg, 41.00%) as a white solid. MS (ESI): mass calcd. for C₂₂H₂₄F₃N₃O₃: 435.18 m/z, found:436.20 [M+H]⁺. ¹H NMR (400 MHz, DMSO-d₆) δ 8.10-8.40 (m, 1H), 7.45-7.72 (m, 4H), 7.13-7.30 (m, 1H), 6.81-6.96 (m, 1H), 6.61-6.81 (m, 1H), 5.87-6.10 (m, 1H), 5.10-5.30 (m, 1H), 4.75-4.92 (m, 1H), 1.40-1.53 (m, 3H), 1.24-1.30 (m, 9H). ¹⁹F NMR (376 MHz, DMSO) δ(ppm): −61.0904 and to yield second enantiomer example 3-tert-butyl-1-[(2S)-2-methyl-3-oxo-4-{[3-(trifluoromethyl)phenyl]methyl}-2H-1,4-benzoxazin-7-yl]urea (14.4 mg, 36.00%) as a white solid. MS (ESI): mass calcd. for C₂₂H₂₄F₃N₃O₃: 435.18 m/z, found:436.20 [M+H]⁺. ¹H NMR (400 MHz, DMSO-d₆) δ 8.10-8.35 (m, 1H), 7.50-7.71 (m, 4H), 7.13-7.30 (m, 1H), 6.82-6.99 (m, 1H), 6.70-6.81 (m, 1H), 5.85-6.02 (m, 1H), 5.09-5.27 (m, 2H), 4.72-4.92 (m, 1H), 1.40-1.56 (m, 3H), 1.24-1.30 (m, 9H). ¹⁹F NMR (376 MHz, DMSO) δ(ppm): −61.0913.

The following examples in Table 4 were made according to the procedure for examples 81 and 82.

TABLE 4

| Structure | Ex. | IUPAC Name | 1H NMR | LCMS |
|---|---|---|---|---|
| 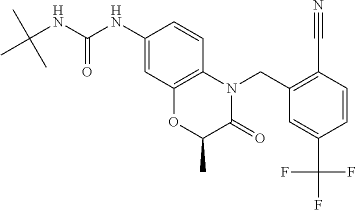 [SMILES: C[C@H]1Oc2cc(NC(=O)NC(C)(C)C)ccc2N(Cc2cc(C(F)(F)F)ccc2C#N)C1=O] | 83 | (R)-1-(tert-butyl)-3-(4- (2-cyano-5- (trifluoromethyl)benzyl)- 2-methyl-3-oxo-3,4- dihydro-2H- benzo[b][1,4]oxazin- 7-yl)urea | ¹H NMR (400 MHz, DMSO-d₆) δ 8.25- 8.46 (m, 1H), 8.09- 8.25 (m, 1H), 7.76- 8.00 (m, 1H), 7.40- 7.62 (m, 1H), 7.11- 7.38 (m, 1H), 6.68- 6.96 (m, 2H), 5.80- 6.14 (m, 1H), 5.20- 5.50 (m, 2H), 4.72- 4.93 (m, 1H), 1.40- 1.56 (m, 3H), 1.24- 1.30 (m, 9H). ¹⁹F | calcd. for C₂₃H₂₃F₃N₄O₃: 460.17 m/z, found 461.20 [M + H]⁺ |
| | | | NMR (376 MHz, | |
| | | | DMSO) δ (ppm): | |
| | | | −62.0673. | |
| 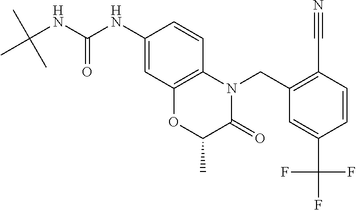 [SMILES: C[C@@H]1Oc2cc(NC(=O)NC(C)(C)C)ccc2N(Cc2cc(C(F)(F)F)ccc2C#N)C1=O] | 84 | (S)-1-(tert-butyl)-3-(4- (2-cyano-5- (trifluoromethyl)benzyl)- 2-methyl-3-oxo-3,4- dihydro-2H- benzo[b][1,4]oxazin- 7-yl)urea | ¹H NMR (400 MHz, DMSO-d₆) δ 8.21- 8.40 (m, 1H), 8.08- 8.21 (m, 1H), 7.73- 8.00 (m, 1H), 7.40- 7.55 (m, 1H), 7.19- 7.34 (m, 1H), 6.66- 7.02 (m, 2H), 5.82- 6.10 (m, 1H), 5.20- 5.50 (m, 2H), 4.67- 4.90 (m, 1H), 1.40- 1.64 (m, 3H), 1.17- 1.36 (m, 9H). ¹⁹F | calcd. for C₂₃H₂₃F₃N₄O₃: 460.17 m/z, found 461.20 [M + H]⁺ |
| | | | NMR (376 MHz, | |
| | | | DMSO) δ (ppm): | |
| | | | −62.0706. | |
| 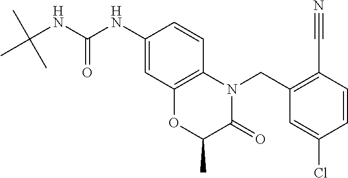 [SMILES: C[C@H]1Oc2cc(NC(=O)NC(C)(C)C)ccc2N(Cc2cc(Cl)ccc2C#N)C1=O] | 85 | (R)-1-(tert-butyl)-3-(4- (5-chloro-2- cyanobenzyl)-2- methyl-3-oxo-3,4- dihydro-2H- benzo[b][1,4]oxazin- 7-yl)urea | ¹H NMR (400 MHz, DMSO-d₆) δ 8.00- 8.41 (m, 1H), 7.82- 8.00 (m, 1H), 7.50- 7.72 (m, 1H), 7.10- 7.40 (m, 2H), 6.56- 6.96 (m, 2H), 5.62- 6.10 (m, 1H), 5.26- 5.41 (m, 1H), 5.00- 5.26 (m, 1H), 4.67- 5.00 (m, 1H), 1.40- | calcd. for C₂₂H₂₃ClN₄O₃ 426.15 m/z, found 427.15 [M + H]⁺ |
| | | | 1.60 (m, 3H), 1.17- | |
| | | | 1.38 (m, 9H). | |
| 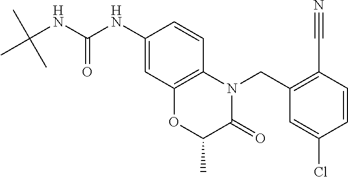 [SMILES: C[C@@H]1Oc2cc(NC(=O)NC(C)(C)C)ccc2N(Cc2cc(Cl)ccc2C#N)C1=O] | 86 | (S)-1-(tert-butyl)-3-(4- (5-chloro-2- cyanobenzyl)-2- methyl-3-oxo-3,4- dihydro-2H- benzo[b][1,4]oxazin- 7-yl)urea | ¹H NMR (400 MHz, DMSO-d₆) δ 8.10- 8.45 (m, 1H), 7.78- 8.10 (m, 1H), 7.50- 7.78 (m, 1H), 7.08- 7.40 (m, 2H), 6.52- 6.90 (m, 2H), 5.66- 6.16 (m, 1H), 5.24- 5.40 (m, 1H), 5.07- 5.24 (m, 1H), 4.69- 5.00 (m, 1H), 1.45- | calcd. for C₂₂H₂₃ClN₄O₃: 426.15 m/z, found 427.20 [M + H]⁺ |
| | | | 1.55 (m, 3H), 1.25- | |
| | | | 1.30 (m, 9H). | |
| 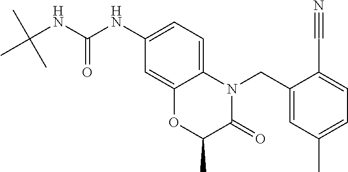 [SMILES: Cc1ccc(C#N)c(CN2C(=O)[C@@H](C)Oc3cc(NC(=O)NC(C)(C)C)ccc32)c1] | 87 | (R)-1-(tert-butyl)-3-(4- (2-cyano-5- methylbenzyl)-2- methyl-3-oxo-3,4- dihydro-2H- benzo[b][1,4]oxazin- 7-yl)urea | ¹H NMR (400 MHz, DMSO-d₆) δ 8.25 (s, 1H), 7.76-7.78 (m, 1H), 7.23-7.33 (m, 2H), 7.02 (s, 1H), 6.72- 6.83 (m, 2H), 5.96 (s, 1H), 5.14-5.27 (m, 2H), 4.83-4.88 (m, 1H), 2.31 (s, 3H), 1.50 (d, J = 6.7 Hz, 3H), 1.27 (s, 9H) | calcd. for C₂₃H₂₆N₄O₃: 406.20 m/z, found: 407.05 [M + H]⁺. |
| 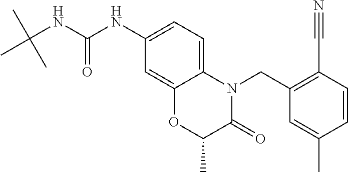 [SMILES: Cc1ccc(C#N)c(CN2C(=O)[C@H](C)Oc3cc(NC(=O)NC(C)(C)C)ccc32)c1] | 88 | (S)-1-(tert-butyl)-3-(4- (2-cyano-5- methylbenzyl)-2- methyl-3-oxo-3,4- dihydro-2H- benzo[b][1,4]oxazin- 7-yl)urea | ¹H NMR (400 MHz, DMSO-d₆) δ 8.24 (s, 1H), 7.76-7.78 (m, 1H), 7.23-7.33 (m, 2H), 7.02 (s, 1H), 6.72- 6.83 (m, 2H), 5.96 (s, 1H), 5.11-5.27 (m, 2H), 4.84-4.88 (m, 1H), 2.31 (s, 3H), 1.50 (d, J = 6.7 Hz, 3H), 1.27 (s, 9H) | calcd. for C₂₃H₂₆N₄O₃: 406.20 m/z, found: 407.05 [M + H]⁺. |

### Example 89: 1-(tert-butyl)-3-(1-(3-chlorobenzyl)-2-oxo-1,2-dihydroquinoxalin-6-yl)urea

### Synthetic Scheme

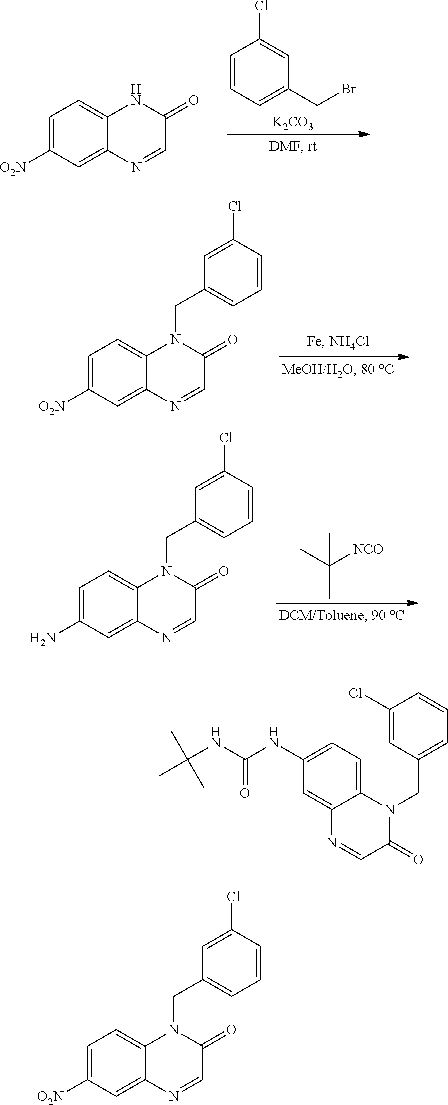

### 1-(3-chlorobenzyl)-6-nitroquinoxalin-2(1H)-one

To a solution of 6-nitro-1H-quinoxalin-2-one (400 mg, 2.093 mmol, 1 equiv) in DMF (10 mL) was added 1-(bromomethyl)-3-chlorobenzene (644.99 mg, 3.139 mmol, 1.5 equiv) and K₂CO₃ (582.65 mg, 4.186 mmol, 2 equiv). The reaction was stirred at rt for 1 hour. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 1-[(3-chlorophenyl)methyl]-6-nitroquinoxalin-2-one (510 mg, 77.19%) as a yellow solid. MS (ESI): mass calcd. for C₁₅H₁₀ClN₃O₃: 315.04m/z, found 316.05 [M+H]⁺.

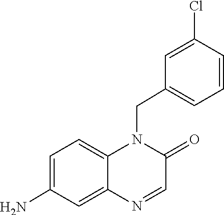 [SMILES: Nc1ccc2c(c1)ncc(=O)n2Cc1cccc(Cl)c1]

### 6-amino-1-(3-chlorobenzyl)quinoxalin-2(1H)-one

To a solution of 1-[(3-chlorophenyl)methyl]-6-nitroquinoxalin-2-one (400 mg, 1.267 mmol, 1 equiv) in MeOH/H₂O (10:1, 11 mL) was added Fe (707.55 mg, 12.670 mmol, 10 equiv) and NH₄Cl (677.71 mg, 12.670 mmol, 10 equiv). The reaction was stirred at 80° C. for 1 hour. The mixture was allowed to cool down to rt. The resulting mixture was filtered, and the filter cake was washed with MeOH (3×20 mL). The filtrate was concentrated under reduced pressure. The mixture was diluted with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 6-amino-1-[(3-chlorophenyl)methyl]quinoxalin-2-one (266 mg, 73.48%) as a yellow solid. MS (ESI): mass calcd. for C₁₅H₁₂ClN₃O: 285.07m/z, found 286.00 [M+H]⁺.

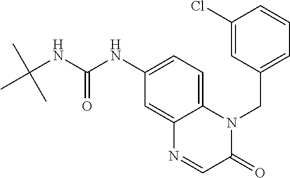 [SMILES: CC(C)(C)NC(=O)Nc1ccc2c(c1)ncc(=O)n2Cc1cccc(Cl)c1]

### 1-(tert-butyl)-3-(1-(3-chlorobenzyl)-2-oxo-1,2-dihydroquinoxalin-6-yl)urea

To a solution of 6-amino-1-[(3-chlorophenyl)methyl]quinoxalin-2-one (130 mg, 0.455 mmol, 1 equiv) in DCM: Toluene (2:1, 15 mL) was added 2-isocyanato-2-methylpropane (270.62 mg, 2.730 mmol, 6 equiv). The reaction was stirred at 90° C. under N₂ for 3 h. After cooling down to rt, the reaction was quenched with water (30 mL) and extracted with DCM (3×30 mL). The combined extracts were washed with water, brine, dried over Na₂SO₄, filtered and concentrated. The residue was purified by preparative HPLC using a XBridge Prep OBD C18 Column, 30*150 mm, 5 μm column (eluent: 17% to 42% (v/v) CH₃CN and H₂O with 10 mmol/L NH₄HCO₃) to afford the title compound 3-tert-butyl-1-{1-[(3-chlorophenyl)methyl]-2-oxoquinoxalin-6-yl}urea (22.2 mg, 12.68%) as a yellow solid. MS (ESI): mass calcd. for C₂₀H₂₁ClN₄O₂: 384.14m/z, found:383.10 [M−H]⁻. ¹H NMR (400 MHz, DMSO-d₆) δ 8.40-8.53 (m, 1H), 8.20-8.40 (m, 1H), 7.91-8.10 (m, 1H), 7.30-7.50 (m, 5H), 7.10-7.27 (m, 1H), 5.80-6.20 (m, 1H), 5.30-5.60 (m, 2H), 1.21-1.40 (m, 9H).

The following examples in Table 5 were made according to the procedure for example 89.

TABLE 5

| Structure | Ex. | IUPAC Name | 1H NMR | LCMS |
|---|---|---|---|---|
| 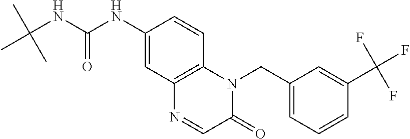 [SMILES: CC(C)(C)NC(=O)Nc1ccc2c(c1)ncc(=O)n2Cc1cccc(C(F)(F)F)c1] | 90 | 1-(tert-butyl)- 3-(2-oxo-1-(3- (trifluoro- methyl) benzyl)-1,2-di- hydro- quinoxalin- 6-yl)urea | ¹H NMR (300 MHz, Methanol-d₄) δ 8.32 (s, 1H), 7.92 (d, J = 2.4 Hz, 1H), 7.64 (s, 1H), 7.56- 7.50 (m, 4H), 7.36 (d, J = 9.3 Hz, 1H), 5.61 (s, 2H), 1.36 (s, 9H). ¹⁹F NMR (282 MHz, Methanol-d4) δ −64.21. | calcd. for C₂₁H₂₁F₃N₄O₂: 418.16 m/z, found 419.15 [M + H]⁺ |
| 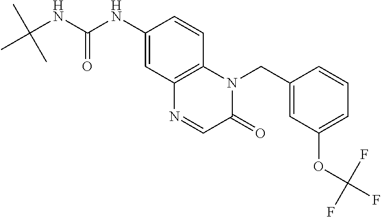 [SMILES: CC(C)(C)NC(=O)Nc1ccc2c(c1)ncc(=O)n2Cc1cccc(OC(F)(F)F)c1] | 91 | 1-(tert-butyl)-3- (2-oxo-1-(3- (trifluoro- methoxy) benzyl)-1,2-di- hydro- quinoxalin- 6-yl)urea | ¹H NMR (300 MHz, DMSO-d6) δ 8.49 (s, 1H), 8.33 (s, 1H), 7.98 (d, J = 2.4 Hz, 1H), 7.51- 7.26 (m, 5H), 7.25 (d, J = 7.5 Hz, 2H), 6.06 (s, 1H), 5.49 (s, 2H), 1.29 (s, 9H). ¹⁹F NMR (376 MHz, DMSO-d₆) δ −56.69. | calcd. for C₂₁H₂₁F₃N₄O₃: 434.16 m/z. found:435.05 [M + H]⁺ |
| 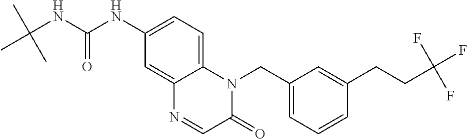 [SMILES: CC(C)(C)NC(=O)Nc1ccc2c(c1)ncc(=O)n2Cc1cccc(CCC(F)(F)F)c1] | 92 | 1-(tert-butyl)-3- (2-oxo-1-(3- (3,3,3-trifluoro- propyl)benzyl)- 1,2-dihydro- quinoxalin- 6-yl)urea | ¹H NMR (300 MHz, DMSO-d₆) δ 8.44 (s, 1H), 8.32 (s, 1H), 7.96 (d, J = 2.4 Hz, 1H), 7.43 (dd, J = 9.1, 2.5 Hz, 1H), 7.07- 7.29 (m, 4H), 7.05 (d, J = 7.3 Hz, 1H), 6.02 (s, 1H), 5.41 (s, 2H), 2.78 (dd, J = 10.1, 6.1 Hz, 2H), 2.58 | calcd. for C₂₃H₂₅F₃N₄O₂, 446.19 m/z, found 447.10 [M + H]⁺ |
| | | | (d, J = 10.8 Hz, 2H), 1.29 | |
| | | | (s, 9H). | |
| 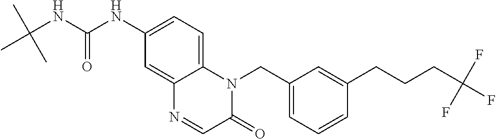 [SMILES: CC(C)(C)NC(=O)Nc1ccc2c(c1)ncc(=O)n2Cc1cccc(CCCC(F)(F)F)c1] | 93 | 1-(tert-butyl)-3- (2-oxo-1-(3- (4,4,4-trifluoro- butyl)benzyl)- 1,2-dihydro- quinoxalin- 6-yl)urea | ¹H NMR (300 MHz, DMSO-d₆) δ 8.44 (s, 1H), 8.32 (s, 1H), 7.98 (d, J = 2.4 Hz, 1H), 7.41 (dd, J = 9.0, 2.5 Hz, 1H), 7.34 (d, J = 9.1 Hz, 1H), 7.25 (t, J = 7.6 Hz, 1H), 7.09- 7.18 (m, 2H), 7.05 (d, J = 7.6 Hz, 1H), 6.02 (s, 1H), | calcd. for C₂₄H₂₇F₃N₄O₂, 460.21 m/z, found 461.15 [M + H]⁺ |
| | | | 5.42 (s, 2H), 2.63 (t, J = | |
| | | | 7.7 Hz, 2H), 2.15-2.24 | |
| | | | (m, 2H), 1.69-1.75 (m, | |
| | | | 2H), 1.29 (s, 9H). | |
| 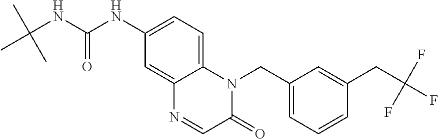 [SMILES: CC(C)(C)NC(=O)Nc1ccc2c(c1)ncc(=O)n2Cc1cccc(CC(F)(F)F)c1] | 94 | 1-(tert-butyl)-3- (2-oxo-1-(3- (2,2,2-trifluoro- ethyl)benzyl)- 1,2-dihydro- quinoxalin- 6-yl)urea | ¹H NMR (400 MHz, DMSO-d₆) δ 8.46 (s, 1H), 8.33 (s, 1H), 7.97 (d, J = 2.5 Hz, 1H), 7.42 (dd, J = 9.1, 2.5 Hz, 1H), 7.29- 7.38 (m, 2H), 7.26 (s, 2H), 7.21-7.25 (m, 1H), 6.03 (s, 1H), 5.45 (s, 2H), 3.60 (q, J = 11.6 Hz, 2H), | calcd. for C₂₂H₂₃F₃N₄O₂, 432.18 m/z, found 431.00 [M − H]⁻ |
| | | | 1.29 (s, 9H). | |
| 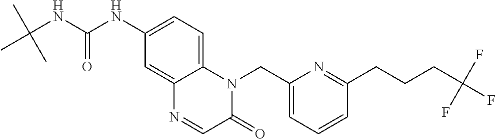 [SMILES: CC(C)(C)NC(=O)Nc1ccc2c(c1)ncc(=O)n2Cc1cccc(CCCC(F)(F)F)n1] | 95 | 1-(tert-butyl)-3- (2-oxo-1-((6- (4,4,4-trifluoro- butyl)pyri- din-2- yl)methyl)-1,2- dihydro- quinoxalin- 6-yl)urea | ¹H NMR (400 MHz, DMSO-d₆) δ 8.44 (s, 1H), 8.30 (s, 1H), 7.97 (d, J = 2.4 Hz, 1H), 7.67 (t, J = 7.7 Hz, 1H), 7.39 (dd, J = 9.1, 2.5 Hz, 1H), 7.32 (d, J = 9.1 Hz, 1H), 7.17 (d, J = 7.6 Hz, 1H), 7.08 (d, J = 7.8 Hz, 1H), 6.03 (s, | calcd. for C₂₃H₂₆F₃N₅O₂, 461.20 m/z, found 462.15 [M + H]⁺ |
| | | | 1H), 5.50 (s, 2H), 2.75 (t, | |
| | | | J = 7.4 Hz, 2H), 2.06- | |
| | | | 2.23 (m, 2H), 1.73-1.80 | |
| | | | (m, 2H), 1.29 (s, 9H). | |
| 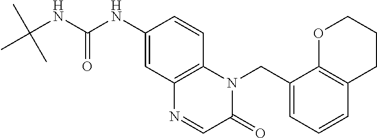 [SMILES: CC(C)(C)NC(=O)Nc1ccc2c(c1)ncc(=O)n2Cc1cccc2c1OCCC2] | 96 | 1-(tert-buty1)-3- (1-(chroman-8- ylmethyl)-2- oxo- 1,2-dihydro- quinoxalin- 6-yl)urea | ¹H NMR (400 MHz, DMSO-d₆) δ 8.46 (s, 1H), 8.31 (s, 1H), 7.89-8.04 (m, 1H),7.38-7.52 (m, 1H), 7.10-7.20 (m, 1H), 6.90-7.02 (m, 1H), 6.60- 6.72 (m, 1H), 6.35-6.48 (m, 1H), 6.00 (s, 1H), 5.11-5.32 (m, 2H), 4.20- | calcd. for C₂₃H₂₆N₄O₃: 406.20, found: 407.05 [M + H]⁺ |
| | | | 4.38 (m, 2H), 2.78-2.85 | |
| | | | (m, 2H), 1.98-2.09 (m, | |
| | | | 2H), 1.25-1.48 (m, 9H) | |
| 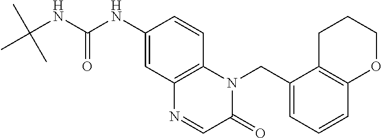 [SMILES: CC(C)(C)NC(=O)Nc1ccc2c(c1)ncc(=O)n2Cc1cccc2c1CCCO2] | 97 | 1-(tert-butyl)-3- (1-(chroman-5- ylmethyl)-2- oxo-1,2-di- hydro- quinoxalin- 6-yl)urea | ¹H NMR (300 MHz, Chloroform-d) δ 8.40 (s, 1H), 7.70 (d, J = 2.5 Hz, 1H), 7.62 (dd, J = 8.9, 2.6 Hz, 1H), 7.28 (s, 2H), 6.87-7.05 (m, 1H), 6.74 (d, J = 8.2 Hz, 1H), 6.26 (s, 1H), 6.19 (d, J = 7.6 Hz, 2H), 5.35 (s, 1H), | calcd. for C₂₃H₂₆N₄O₃: 406.20 m/z found: 407.05 [M + H]⁺ |
| | | | 4.18-4.27 (m, 2H), 2.85 | |
| | | | (t, J = 6.6 Hz, 2H), 2.10- | |
| | | | 2.20 (m, 2H), 1.40 (s, | |
| | | | 9H). | |

### Example 98: (S)-1-(tert-butyl)-3-(2-oxo-1-(1-phenylethyl)-1,2-dihydroquinoxalin-6-yl)urea

### Synthetic Scheme

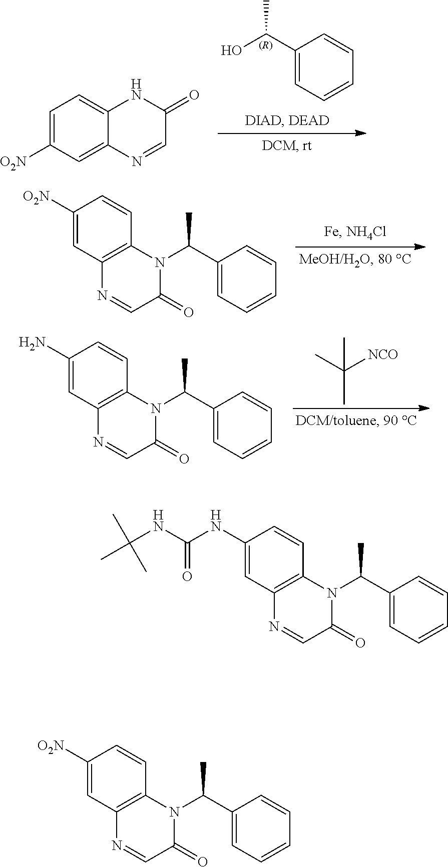

### (S)-6-nitro-1-(1-phenylethyl)quinoxalin-2(1H)-one

To a solution of 6-nitro-1H-quinoxalin-2-one (400 mg, 2.093 mmol, 1 equiv) in DCM (10 mL) was added (R)-1-phenylethan-1-ol (255.65 mg, 2.093 mmol, 1 equiv) and PPh₃ (823.32 mg, 3.139 mmol, 1.5 equiv). The reaction was stirred at rt under N₂ for 0.5h. To the above mixture was added DEAD (546.67 mg, 3.139 mmol, 1.5 equiv) in portions at 0° C. The resulting mixture was stirred for additional 1 hour at rt. Quenched with water (30 mL) and extracted with DCM (3×30 mL). The combined extracts were washed with water, brine, dried over Na₂SO₄, filtered and concentrated. The residue was purified by column chromatography on silica gel with EA/PE (0-100%) to 6-nitro-1-[(1S)-1-phenylethyl]quinoxalin-2-one (108 mg, 17.48%) as a yellow solid. MS (ESI): mass calcd. for C₁₆H₁₃N₃O₃: 295.10m/z, found 296.00 [M+H]⁺.

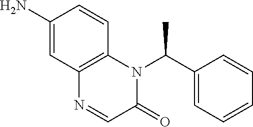 [SMILES: C[C@@H](c1ccccc1)n1c(=O)cnc2cc(N)ccc21]

### (S)-6-amino-1-(1-phenylethyl)quinoxalin-2(1H)-one

To a solution of 6-nitro-1-[(1S)-1-phenylethyl]quinoxalin-2-one (100 mg, 0.339 mmol, 1 equiv) in MeOH/H₂O (10:1, 11 mL) was added Fe (189.11 mg, 3.390 mmol, 10 equiv) and NH₄C₁ (181.14 mg, 3.390 mmol, 10 equiv). The reaction was stirred at 80° C. for 1 hour. The mixture was allowed to cool down to rt. The resulting mixture was filtered, and the filter cake was washed with MeOH (3×20 mL). The filtrate was concentrated under reduced pressure. The mixture was diluted with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give 6-amino-1-[(1S)-1-phenylethyl]quinoxalin-2-one (85 mg, 94.61%) as a yellow solid. MS (ESI): mass calcd. for C₁₆H₁₅N₃O: 265.12m/z, found 266.15 [M+H]⁺.

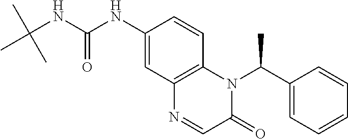 [SMILES: C[C@@H](c1ccccc1)n1c(=O)cnc2cc(NC(=O)NC(C)(C)C)ccc21]

### (S)-1-(tert-butyl)-3-(2-oxo-1-(1-phenylethyl)-1,2-dihydroquinoxalin-6-yl)urea

To a solution of 6-amino-1-[(1S)-1-phenylethyl]quinoxalin-2-one (85 mg, 0.320 mmol, 1 equiv) in DCM: Toluene (2:1, 15 mL) was added 2-isocyanato-2-methylpropane (190.56 mg, 1.920 mmol, 6 equiv). The reaction was stirred at 90° C. under N₂ for 3 hours. After cooling down to rt, the reaction was quenched with water (30 mL) and extracted with DCM (3×30 mL). The combined extracts were washed with water, brine, dried over Na₂SO₄, filtered and concentrated. The residue was purified by preparative HPLC using a XBridge Shield RP18 OBD Column, 30*150 mm, 5 μm column (eluent: 34% to 56% (v/v) CH₃CN and H₂O with 10 mmol/L NH₄HCO₃) to afford the title compound 3-tert-butyl-1-{2-oxo-1-[(1S)-1-phenylethyl]quinoxalin-6-yl}urea (22.1 mg, 18.93%) as a yellow solid. MS (ESI): mass calcd. for C₂₁H₂₄N₄O₂: 364.19m/z, found:365.20 [M+H]⁺. ¹H NMR (400 MHz, DMSO-d₆) δ 8.31-8.50 (m, 1H), 8.17-8.31 (m, 1H), 7.77-8.02 (m, 1H), 7.20-7.40 (m, 6H), 6.86-7.20 (m, 1H), 6.50-6.80 (m, 1H), 5.67-6.20 (m, 1H), 1.76-1.95 (m, 1H), 1.17-1.32 (m, 9H).

The following examples in Table 6 were made according to the procedure for example 98.

TABLE 6

| Structure | Ex. | IUPAC Name | 1H NMR | LCMS |
|---|---|---|---|---|
| 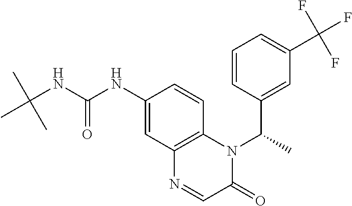 [SMILES: C[C@@H](c1cccc(C(F)(F)F)c1)n1c(=O)cnc2cc(NC(=O)NC(C)(C)C)ccc21] | 99 | (S)-1-(tert-butyl)-3-(2- oxo-1-(1-(3- (trifluoromethyl)phenyl) ethyl)-1,2- dihydroquinoxalin-6- yl)urea | ¹H NMR (300 MHz, DMSO-d₆) δ 8.43 (s, 1H), 8.24 (s, 1H), 7.94 (s, 1H), 7.60-7.70 (m, 4H), 7.31-7.38 (m, 1H), 7.14 (s, 1H), 6.59 (s, 1H), 6.04 (s, 1H), 1.91- 1.93 (d, J = 6 Hz, 3H), 1.29 (s, 9H). ¹⁹F NMR (282 MHz, DMSO) δ −60.88. | calcd. for C₂₂H₂₃F₃N₄O₂: 432.18 m/z, found: 433.00 [M + H]⁺ |
| 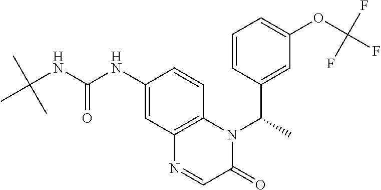 [SMILES: C[C@@H](c1cccc(OC(F)(F)F)c1)n1c(=O)cnc2cc(NC(=O)NC(C)(C)C)ccc21] | 100 | (S)-1-(tert-butyl)-3-(2- oxo-1-(1-(3- (trifluoromethoxy)phe- nyl)ethyl)-1,2- dihydroquinoxalin-6- yl)urea | ¹H NMR (300 MHz, DMSO-d₆) δ 8.42 (s, 1H), 8.24 (s, 1H), 7.94 (d, J = 2.4 Hz, 1H), 7.48 (t, J = 8.1 Hz, 1H), 7.31- 7.27 (m, 4H), 7.09 (s, 1H), 6.03 (s, 1H), 1.88 (d, J = 7.2 Hz, 3H), 1.29 (s, 9H). ¹⁹F NMR (282 MHz, DMSO-d₆) δ −56.68. | calcd. for C₂₂H₂₃F₃N₄O₃: 448.1 m/z, found: 449.15 [M + H]⁺ |
| 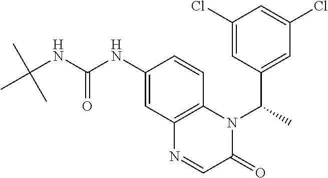 [SMILES: C[C@@H](c1cc(Cl)cc(Cl)c1)n1c(=O)cnc2cc(NC(=O)NC(C)(C)C)ccc21] | 101 | (S)-1-(tert-butyl)-3-(1- (1-(3,5- dichlorophenyl)ethyl)- 2-oxo-1,2- dihydroquinoxalin-6- yl)urea | ¹H NMR (300 MHz, DMSO-d₆) δ 8.45 (s, 1H), 8.23 (s, 1H), 7.95 (d, J = 2.4 Hz, 1H), 7.54 (d, J = 2.4 Hz, 1H), 7.34- 7.26 (m, 3H), 7.06 (s, 1H), 6.49 (s, 1H), 6.05 (s, 1H), 1.86 (d, J = 7.2 Hz, 3H), 1.29 (s, 9H). | calcd. for C₂₁H₂₂Cl₂N₄O₂: 432.1 m/z, found: 433.10 [M + H]⁺. |
| 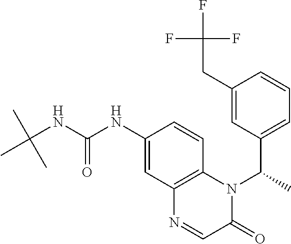 [SMILES: C[C@@H](c1cccc(CC(F)(F)F)c1)n1c(=O)cnc2cc(NC(=O)NC(C)(C)C)ccc21] | 102 | (S)-1-(tert-butyl)-3-(2- oxo-1-(1-(3-(2,2,2- trifluoroethyl)phe- nyl)ethyl)-1,2- dihydroquinoxalin-6- yl)urea | ¹H NMR (400 MHz, DMSO-d₆) δ 8.40 (s, 1H), 8.26 (s, 1H), 7.92 (d, J = 2.6 Hz, 1H), 7.37 (t, J = 7.9 Hz, 1H), 7.23- 7.31 (m, 4H), 7.07 (s, 1H), 6.60 (s, 1H), 3.61 (q, J = 11.6 Hz, 2H), 1.88 (d, J = 7.1 Hz, 3H), 1.28 (s, 9H). | calcd. for C₂₃H₂₅F₃N₄O₂, 446.19 m/z, found 447.10 [M + H]⁺ |
| 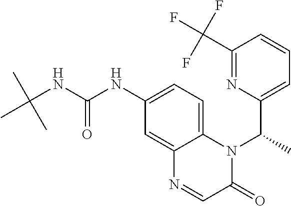 [SMILES: C[C@@H](c1cccc(C(F)(F)F)n1)n1c(=O)cnc2cc(NC(=O)NC(C)(C)C)ccc21] | 103 | (S)-1-(tert-butyl)-3-(2- oxo-1-(1-(6-(tri- fluoromethyl)pyridin- 2-yl)ethyl)-1,2- dihydroquinoxalin-6- yl)urea | ¹H NMR (400 MHz, DMSO-d₆) δ 8.36-8.50 (m, 1H), 8.14-8.27 (m, 1H), 7.98-8.10 (m, 1H), 7.89-7.98 (m, 1H), 7.70-7.89 (m, 1H), 7.52-7.66 (m, 1H), 7.32-7.45 (m, 1H), 6.95-7.32 (m, 1H), 6.34-6.78 (m, 1H), 5.90-6.15 (m, 1H), 1.80-1.99 (m, 3H), 1.24-1.35 (m, | calcd. for C₂₁H₂₂F₃N₅O₂: 433.17 m/z, found 434.20 [M + H]⁺ |
| | | | 9H). ¹⁹F NMR (376 | |
| | | | MHz, DMSO) δ (ppm): | |
| | | | −66.5322. | |
| 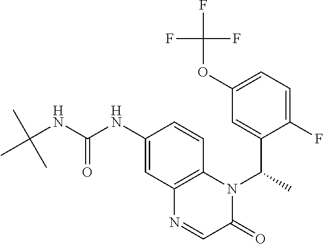 [SMILES: C[C@@H](c1cc(OC(F)(F)F)ccc1F)n1c(=O)cnc2cc(NC(=O)NC(C)(C)C)ccc21] | 104 | (S)-1-(tert-butyl)-3-(1- (1-(2-fluoro-5- (trifluoromethoxy)phe- nyl)ethyl)-2-oxo-1,2- dihydroquinoxalin-6- yl)urea | ¹H NMR (400 MHz, DMSO-d₆) δ 8.48 (s, 1H), 8.13 (s, 1H), 7.90 (d, J = 2.5 Hz, 1H), 7.69 (s, 1H), 7.56 (s, 1H), 7.47 (s, 1H), 7.39 (d, J = 8.9 Hz, 1H), 7.23 (t, J = 9.5 Hz, 1H), 6.48 (s, 1H), 6.04 (s, 1H), 1.87 (d, J = 6.9 Hz, 3H), 1.30 (s, 9H). | calcd. for C₂₂H₂₂F₄N₄O₃, 466.16 m/z, found 467.10 [M + H]⁺ |
| 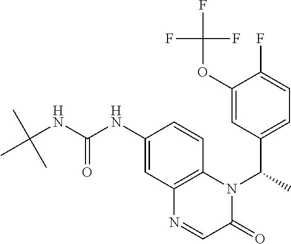 [SMILES: C[C@@H](c1ccc(F)c(OC(F)(F)F)c1)n1c(=O)cnc2cc(NC(=O)NC(C)(C)C)ccc21] | 105 | (S)-1-(tert-buty1)-3-(1- (1-(4-fluoro-3- (trifluoromethoxy)phe- nyl)ethyl)-2-oxo-1,2- dihydroquinoxalin-6- yl)urea | ¹H NMR (400 MHz, DMSO-d₆) δ 8.44 (s, 1H), 8.23 (s, 1H), 7.93 (d, J = 2.5 Hz, 1H), 7.42- 7.53 (m, 2H), 7.31- 7.42 (m, 2H), 7.14 (br, 1H), 6.52 (br, 1H), 6.03 (s, 1H), 1.88 (d, J = 7.0 Hz, 3H), 1.29 (s, 9H). | calcd. For C₂₂H₂₂F₄N₄O₃: 466.16 m/z, found: 467.10 [M + H]⁺. |
| 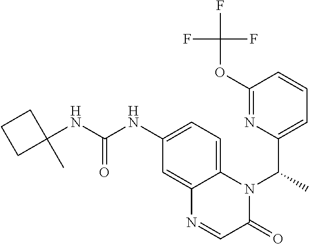 [SMILES: C[C@@H](c1cccc(OC(F)(F)F)n1)n1c(=O)cnc2cc(NC(=O)NC3(C)CCC3)ccc21] | 106 | (S)-1-(1- methylcyclobutyl)-3- (2-oxo-1-(1-(6- (trifluoromethoxy)pyri- din-2-yl)ethyl)-1,2- dihydroquinoxalin-6- yl)urea | ¹H NMR (400 MHz, DMSO-d₆) δ 8.45 (s, 1H), 8.22 (s, 1H), 7.95- 8.05 (m, 1H), 7.93 (d, J = 2.5 Hz, 1H), 7.39 (d, J = 7.7 Hz, 2H), 7.02- 7.29 (m, 2H), 6.50 (s, 1H), 6.33 (s, 1H), 2.21- 2.34 (m, 2H), 1.82- 1.93 (m, 5H), 1.70- 1.82 (m, 2H), 1.40 (s, 3H). ¹⁹F NMR (376 MHz, DMSO) δ (ppm): −55.2989. | calcd. for C₂₂H₂₂F₃N₅O₃: 461.17 m/z, found 462.15 [M + H]⁺ |
| 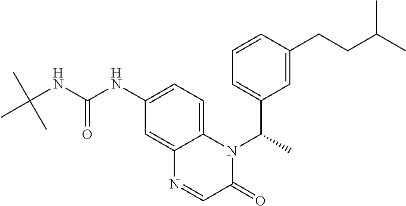 [SMILES: CC(C)CCc1cccc([C@H](C)n2c(=O)cnc3cc(NC(=O)NC(C)(C)C)ccc32)c1] | 107 | (S)-1-(tert-butyl)-3-(1- (1-(3- isopentylphenyl)ethyl)- 2-oxo-1,2- dihydroquinoxalin-6- yl)urea | ¹H NMR (400 MHz, DMSO-d₆) δ 8.38 (s, 1H), 8.27 (s, 1H), 7.92 (d, J = 2.6 Hz, 1H), 7.25 (t, J = 7.7 Hz, 2H), 7.00- 7.12 (m, 4H), 6.59 (s, 1H), 6.01 (s, 1H), 2.55 (s, 2H), 1.87 (d, J = 7.1 Hz, 3H), 1.33-1.44 (m, 3H), 1.28 (s, 9H), 0.76-0.88 (m, 6H). | calcd. for C₂₆H₃₄N₄O₂: 434.27 m/z, found: 435.20 [M + H]⁺ |
| 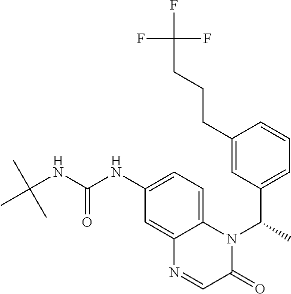 [SMILES: C[C@@H](c1cccc(CCCC(F)(F)F)c1)n1c(=O)cnc2cc(NC(=O)NC(C)(C)C)ccc21] | 108 | (S)-1-(tert-butyl)-3-(2- oxo-1-(1-(3-(4,4,4- trifluorobutyl)phenyl) ethyl)-1,2- dihydroquinoxalin-6- yl)urea | ¹H NMR (400 MHz, DMSO-d₆) δ 8.38 (s, 1H), 8.27 (s, 1H), 7.93 (d, J = 2.6 Hz, 1H), 7.20- 7.32 (m, 2H), 7.08- 7.16 (m, 3H), 7.04 (s, 1H), 6.59 (s, 1H), 6.02 (s, 1H), 2.62 (t, J = 7.6 Hz, 2H), 2.05-2.19 (m, 2H), 1.88 (d, J = 7.2 Hz, 3H), 1.65-1.78 (m, 2H), 1.28 (s, 9H). | calcd. for C₂₅H₂₉F₃N₄O₂: 474.22 m/z, found: 475.20 [M + H]⁺ |
| 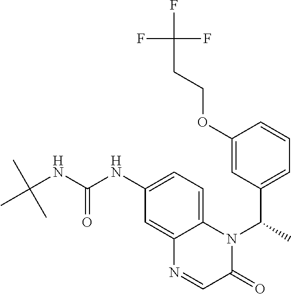 [SMILES: C[C@@H](c1cccc(OCCC(F)(F)F)c1)n1c(=O)cnc2cc(NC(=O)NC(C)(C)C)ccc21] | 109 | (S)-1-(tert-butyl)-3-(2- oxo-1-(1-(3-(3,3,3- trifluoropropoxy) phenyl)ethyl)-1,2- dihydroquinoxalin-6- yl)urea | ¹H NMR (400 MHz, DMSO-d₆) δ 8.39 (s, 1H), 8.26 (s, 1H), 7.93 (d, J = 2.5 Hz, 1H), 7.27 (t, J = 7.8 Hz, 2H), 7.06 (s, 1H), 6.80-6.93 (m, 3H), 6.57 (s, 1H), 6.02 (s, 1H), 4.11-4.24 (m, 2H), 2.66-2.83 (m,2H), 1.87 (d, J = 7.1 Hz, 3H), 1.28 (s, 9H). | calcd. for C₂₄H₂₇F₃N₄O₃: 476.20 m/z, found: 477.20 [M + H]⁺ |
| 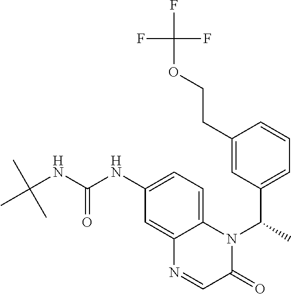 [SMILES: C[C@@H](c1cccc(CCOC(F)(F)F)c1)n1c(=O)cnc2cc(NC(=O)NC(C)(C)C)ccc21] | 110 | (S)-1-(tert-butyl)-3-(2- oxo-1-(1-(3-(2-(tri- fluoromethoxy)ethyl) phenyl)ethyl)-1,2- dihydroquinoxalin-6- yl)urea | ¹H NMR (300 MHz, DMSO-d₆) δ 8.38 (s, 1H), 8.27 (s, 1H), 7.93 (d, J = 2.5 Hz, 1H), 7.17- 7.35 (m, 4H), 7.09- 7.17 (m,1H),7.05 (s, 1H), 6.60 (s, 1H), 6.00 (s, 1H), 4.25 (t, J = 6.5 Hz, 2H), 2.96 (t, J = 6.5 Hz, 2H), 1.88 (d, J = 7.2 Hz, 3H), 1.28 (s, 9H). | calcd. for C₂₄H₂₇F₃N₄O₃: 476.20 M/Z, found: 477.15 [M + H]⁺ |
| 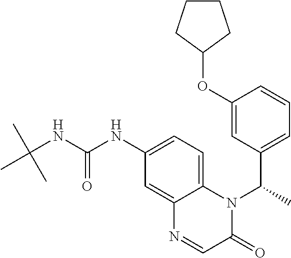 [SMILES: C[C@@H](c1cccc(OC2CCCC2)c1)n1c(=O)cnc2cc(NC(=O)NC(C)(C)C)ccc21] | 111 | (S)-1-(tert-butyl)-3-(1- (1-(3-(cyclo- pentyloxy)phenyl) ethyl)-2-oxo-1,2- dihydroquinoxalin-6- yl)urea | ¹H NMR (400 MHz, DMSO-d₆) δ 8.40 (s, 1H), 8.26 (s, 1H), 7.20- 7.80 (m, 1H), 7.18-7.38 (m, 2H), 6.91-7.18 (m, 1H), 6.78-6.90 (m, 2H), 6.70-6.79 (m, 1H), 6.55 (s, 1H), 6.02 (s, 1H), 4.51-4.91 (m, 1H), 1.78- 1.98 (m, 5H), 1.51-1.71 (m, 6H), 1.22-1.33 (m, 9H) | calcd. for C₂₆H₃₂N₄O₃: 448.25, found: 449.10 [M + H]⁺. |
| 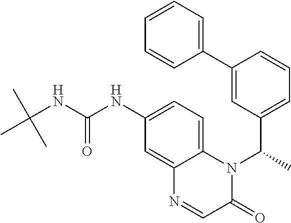 [SMILES: C[C@@H](c1cccc(-c2ccccc2)c1)n1c(=O)cnc2cc(NC(=O)NC(C)(C)C)ccc21] | 112 | (S)-1-(1-(1-([1,1′- biphenyl]-3-yl)ethyl)- 2-oxo-1,2- dihydroquinoxalin-6- yl)-3-(tert-butyl)urea | ¹H NMR (400 MHz, DMSO-d₆) δ 8.33 (d, J = 45.8 Hz, 2H), 7.94 (d, J = 2.5 Hz, 1H), 7.60- 7.67 (m, 2H), 7.54- 7.60 (m, 2H), 7.33- 7.50 (m, 4H), 7.10- 7.32 (m, 3H), 6.66 (s, 1H), 6.02 (s, 1H), 1.95 (d, J = 7.1 Hz, 3H), 1.28 (s, 9H). | calcd. for C₂₇H₂₈N₄O₂: 440.20 m/z, found 441.20 [M + H]⁺ |
| 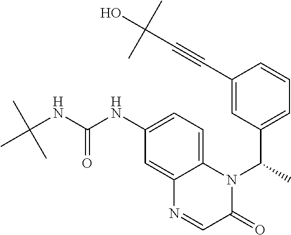 [SMILES: C[C@@H](c1cccc(C#CC(C)(C)O)c1)n1c(=O)cnc2cc(NC(=O)NC(C)(C)C)ccc21] | 113 | (S)-1-(tert-butyl)-3-(1- (1-(3-(3-hydroxy-3- methylbut-1-yn-1- yl)phenyl)ethyl)-2- oxo-1,2- dihydroquinoxalin-6- yl)urea | ¹H NMR (400 MHz, Chloroform-d) δ 8.38 (s, 1H), 7.60 (s, 1H), 7.47- 7.50 (d, J = 12 Hz, 1H), 7.23-7.35(m, 2H), 7.17- 7.19 (m, 1H), 6.97-6.99 (d, J = 8.0 Hz, 1H), 6.80 (s, 1H), 6.15 (s, 1H), 4.51 (s, 1H), 2.07 (s, 1H), 1.92-1.94 (d, J = 8.0 Hz, 3H), 1.57- 1.63(m, 6H), 1.39 (s, 9H). | C₂₆H₃₀N₄O₃: 446.23 m/z, found: 469.15 [M + Na]⁺ |
| 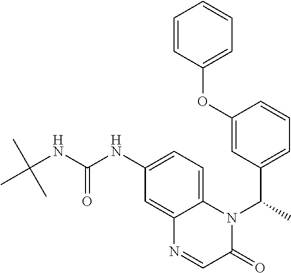 [SMILES: C[C@@H](c1cccc(Oc2ccccc2)c1)n1c(=O)cnc2cc(NC(=O)NC(C)(C)C)ccc21] | 114 | (S)-1-(tert-butyl)-3-(2- oxo-1-(1-(3- phenoxyphenyl)ethyl)- 1,2- dihydroquinoxalin-6- yl)urea | ¹H NMR (400 MHz, DMSO-d₆) δ 8.44 (s, 1H), 8.24 (s, 1H), 7.93 (d, J = 2.5 Hz, 1H), 7.40- 7.29 (m, 4H), 7.15- 7.02 (m, 3H), 6.97- 6.83 (m, 4H), 6.59 (s, 1H), 6.05 (s, 1H), 1.86 (d, J = 7.1 Hz, 3H), 1.29 (s, 9H). | calcd. for C₂₇H₂₈N₄O₃: 456.22 m/z, found 457.20 [M + H]⁺ |
| 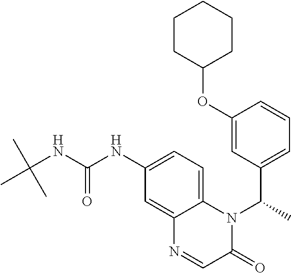 [SMILES: C[C@@H](c1cccc(OC2CCCCC2)c1)n1c(=O)cnc2cc(NC(=O)NC(C)(C)C)ccc21] | 115 | (S)-1-(tert-butyl)-3-(1- (1-(3- (cyclohexyloxy)phenyl) ethyl)-2-oxo-1,2- dihydroquinoxalin-6- yl)urea | ¹H NMR (400 MHz, DMSO-d₆) δ 8.40 (s, 1H), 8.26 (s, 1H), 7.80- 8.04 (m, 1H), 7.19-7.35 (m, 2H), 6.90-7.13 (m, 1H), 6.68-6.90 (m, 3H), 6.41-6.68 (m, 1H), 6.03 (s, 1H), 4.16-4.35 (m, 1H), 1.83-1.92 (m, 4H), 1.47-1.75 (m, 3H), 1.16- 1.42 (m, 15H). | calcd. for C₂₇H₃₄N₄O₃: 462.2 m/z, found 463.10 [M + H]⁺. |
| 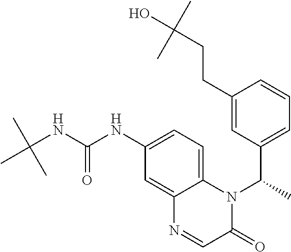 [SMILES: C[C@@H](c1cccc(CCC(C)(C)O)c1)n1c(=O)cnc2cc(NC(=O)NC(C)(C)C)ccc21] | 116 | (S)-1-(tert-butyl)-3-(1- (1-(3-(3-hydroxy-3- methylbutyl)phenyl) ethyl)-2-oxo-1,2- dihydroquinoxalin-6- yl)urea | ¹H NMR (300 MHz, DMSO-d₆) δ 8.36 (s, 1H), 8.26 (s, 1H), 7.92 (d, J = 2.4 Hz, 1H), 7.22 (t, J = 7.8 Hz, 2H), 7.14- 7.01 (m, 3H), 6.57 (s, 1H), 6.02 (s, 1H), 5.44 (s, 1H), 4.22 (s, 1H), 2.51 (d, J = 1.8 Hz, 2H), 1.87 (d, J = 7.2 Hz, 2H), 1.27-1.10 (m, 18H). | calcd. for C₂₆H₃₄N₄O₃: 450.3 m/z, found 451.2 [M + H]⁺ |
| 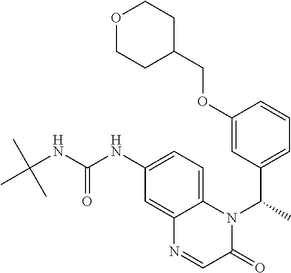 [SMILES: C[C@@H](c1cccc(OCC2CCOCC2)c1)n1c(=O)cnc2cc(NC(=O)NC(C)(C)C)ccc21] | 117 | (S)-1-(tert-butyl)-3-(2- oxo-1-(1-(3- ((tetrahydro-2H- pyran-4- yl)methoxy)phenyl)eth- yl)-1,2- dihydroquinoxalin-6- yl)urea | ¹H NMR (400 MHz, DMSO-d₆) δ 8.48 (s, 1H), 8.27 (s,1H), 7.85- 8.02 (m, 1H), 7.20-7.38 (m, 2H), 6.94-7.12 (m, 1H), 6.72-6.90 (m, 3H),6.49-6.68 (m, 1H), 6.10 (s, 1H), 3.71-3.91 (m, 4H), 3.20-3.30 (m, 2H), 1.82-1.90 (m, 3H), 1.58-1.69 (m, 2H), 1.19- 1.39 (m, 12H). | calcd. for C₂₇H₃₄N₄O₄: 478.26, found: 479.25 [M + H]⁺ |
| 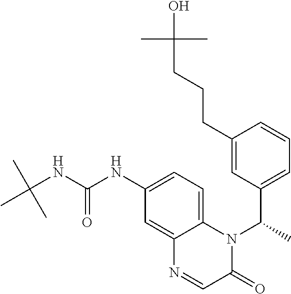 [SMILES: C[C@@H](c1cccc(CCCC(C)(C)O)c1)n1c(=O)cnc2cc(NC(=O)NC(C)(C)C)ccc21] | 118 | (S)-1-(tert-butyl)-3-(1- (1-(3-(4-hydroxy-4- methylpentyl)phenyl) ethyl)-2-oxo-1,2- dihydroquinoxalin-6- yl)urea | ¹H NMR (400 MHz, Chloroform-d) δ 8.37 (s, 1H), 7.54 (d, J = 2.6 Hz, 1H), 7.50 (dd, J = 9.2, 2.6 Hz, 1H), 7.25 (d, J = 7.6 Hz, 1H), 7.10 (d, J = 7.9 Hz, 2H), 7.02 (d, J = 9.2 Hz, 2H), 6.85 (d, J = 7.7 Hz, 1H), 6.28 (s, 1H), 4.59 (s, 1H), 2.57 (t, J = 7.5 Hz, 2H), 1.93 (d, J = 7.2 Hz, 3H), 1.55- 1.68 (m, 4H), 1.38 (s, 9H), 1.17 (d, J = 5.3 Hz, 6H). | calcd. for C₂₇H₃₆N₄O₃: 464.28 m/z found: 465.30 [M + H]⁺ |
| 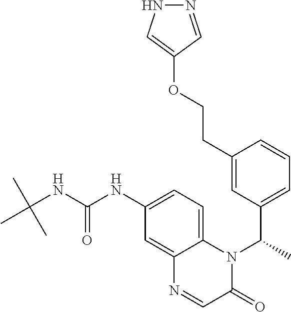 [SMILES: C[C@@H](c1cccc(CCOc2cn[nH]c2)c1)n1c(=O)cnc2cc(NC(=O)NC(C)(C)C)ccc21] | 119 | (S)-1-(1-(1-(3-(2- ((1H-pyrazol-4- yl)oxy)ethyl)phenyl) ethyl)-2-oxo-1,2- dihydroquinoxalin-6- yl)-3-(tert-butyl)urea | ¹H NMR (300 MHz, DMSO-d₆) δ 12.3 (s, 1H), 8.39 (s, 1H), 8.26 (s, 1H), 7.93 (d, J = 2.1 Hz, 1H), 7.29-7.18 (m, 6H), 7.09 (d, J = 7.2 Hz, 2H), 6.59 (s, 1H), 6.02 (s, 1H), 4.00 (t, J = 6.6 Hz, 2H), 2.94 (t, J = 6.8 Hz, 2H), 1.87 (d, J = 7.2 Hz, 3H), 1.26 (s, 9H). | calcd. for C₂₆H₃₀N₆O₃: 474.2 m/z, found 475.2 [M + H]⁺ |
| 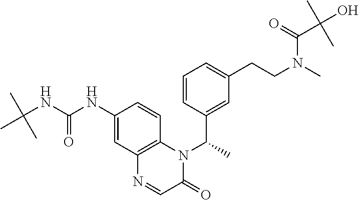 [SMILES: C[C@@H](c1cccc(CCN(C)C(=O)C(C)(C)O)c1)n1c(=O)cnc2cc(NC(=O)NC(C)(C)C)ccc21] | 120 | (S)-N-(3-(1-(6-(3- (tert-butyl)ureido)-2- oxoquinoxalin-1 (2H)- yl)ethyl)phenethyl)-2- hydroxy-N,2- dimethylpropanamide | ¹H NMR (300 MHz, DMSO-d₆) δ 8.39 (s, 1H), 8.27 (s, 1H), 7.90- 8.04 (m, 1H), 7.23- 7.29 (m, 2H), 7.04- 7.19 (m, 4H), 6.50- 6.57 (m, 1H), 6.02 (s, 1H), 5.24-5.34 (m, 1H), 3.78-3.84 (m, 1H), 3.39-3.44 (m, 1H), 3.09-3.15 (m, 2H), 2.79-2.85 (s, 3H), 1.88 (d, J = 7.1 Hz, 3H), | calcd. for C₂₈H₃₇N₅O₄: 507.28 m/z, found 508.35 [M + H]⁺ |
| | | | 1.28 (s, 9H), 1.26 (s, | |
| | | | 6H). | |
| 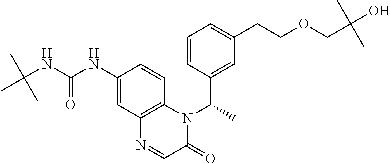 [SMILES: C[C@@H](c1cccc(CCOCC(C)(C)O)c1)n1c(=O)cnc2cc(NC(=O)NC(C)(C)C)ccc21] | 121 | (S)-1-(tert-butyl)-3-(1- (1-(3-(2-(2-hydroxy-2- methylpropoxy)ethyl) phenyl)ethyl)-2-oxo- 1,2- dihydroquinoxalin-6- yl)urea | ¹H NMR (300 MHz, Chloroform-d) δ 8.34 (s, 1H), 7.47-7.58 (m, 2H), 7.27 (d, J = 7.8 Hz, 1H), 7.06-7.17 (m, 3H), 7.01 (d, J = 9.1 Hz, 1H), 6.80 (d, J = 7.2 Hz, 1H), 6.64 (s, 1H), 3.65 (t, J = 6.7 Hz, 2H), 3.23 (s, 2H), 2.85 (t, J = 6.7 Hz, 2H), 1.91 (d, J = 7.2 | calcd. for C₂₇H₃₆N₄O₄: 480.27 m/z found: 479.20 [M − H]⁻ |
| | | | Hz, 3H), 1.45 (d, J = 3.6 | |
| | | | Hz, 9H). 1.17 (d, J = 3.6 | |
| | | | Hz, 6H). | |
| 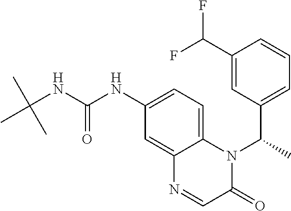 [SMILES: C[C@@H](c1cccc(C(F)F)c1)n1c(=O)cnc2cc(NC(=O)NC(C)(C)C)ccc21] | 122 | (S)-1-(tert-butyl)-3-(1- (1-(3-(difluoro- methyl)phenyl) ethyl)-2-oxo-1,2- dihydroquinoxalin-6- yl)urea | ¹H NMR (400 MHz, DMSO-d₆) δ 8.41 (s, 1H), 8.26 (s, 1H), 7.94 (d, J = 2.5 Hz, 1H), 7.42- 7.55 (m, 4H), 7.30 (s, 1H), 6.80-7.21 (m, 2H), 6.60 (s, 1H), 6.03 (s, 1H), 1.91 (d, J = 7.1 Hz, 3H), 1.28 (s, 9H). ¹⁹F NMR (376 MHz, DMSO) δ (ppm): −109.2872. | calcd. for C₂₂H₂₄F₂N₄O₂: 414.19 m/z, found 415.05 [M + H]⁺ |
| 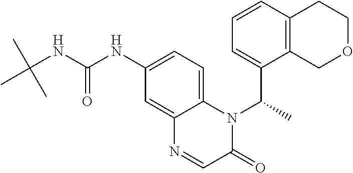 [SMILES: C[C@@H](c1cccc2c1COCC2)n1c(=O)cnc2cc(NC(=O)NC(C)(C)C)ccc21] | 123 | (S)-1-(tert-butyl)-3-(1- (1-(isochroman-8- yl)ethyl)-2-oxo-1,2- dihydroquinoxalin-6- yl)urea | ¹H NMR (300 MHz, DMSO-d₆) δ 8.41 (s, 1H), 8.20 (s, 1H), 7.88 (s, 1H), 7.57-7.60 (m, 1H), 7.41-7.43 (m, 1H), 7.28-7.33 (m, 2H), 7.08- 7.10 (m, 1H), 6.29 (s, 1H), 6.04 (s, 1H), 4.45- 4.50 (d, J = 15.0 Hz, 1H), 3.80-3.92 (m, 1H), 3.62-3.67 (d, J = | calcd. for C₂₄H₂₈N₄O₃: 420.22 m/z, found: 421.10 [M + H]⁺. calcd. |
| | | | 15.0 Hz, 1H), 3.43-3.44 | |
| | | | (m, 1H), 2.65-2.72 (m, | |
| | | | 1H), 2.64-2.67 (m, 1H), | |
| | | | 1.82-1.84 (d, J = 6.0 Hz, | |
| | | | 3H), 1.28 (s, 9H) | |
| 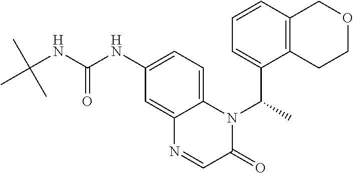 [SMILES: C[C@@H](c1cccc2c1CCOC2)n1c(=O)cnc2cc(NC(=O)NC(C)(C)C)ccc21] | 124 | (S)-1-(tert-butyl)-3-(1- (1-(isochroman-5- yl)ethyl)-2-oxo-1,2- dihydroquinoxalin-6- yl)urea | ¹H NMR (400 MHz, Chloroform-d) δ 8.33 (s, 1H), 7.59 (d, J = 7.9 Hz, 1H), 7.47-7.56 (m, 2H), 7.25-7.37 (m, 2H), 6.97 (d, J = 7.6 Hz, 1H), 6.67 (d, J = 7.3 Hz, 1H), 6.11 (s, 1H), 4.72 (s, 2H), 4.50 (s, 1H), 3.98 (d, J = 11.9 Hz, 1H), 3.57-3.66 (m, | for C₂₄H₂₈N₄O₃: 420.22 m/z found: 443.10 [M + Na]⁺ |
| | | | 1H), 2.50 (d, J = 16.4 | |
| | | | Hz, 1H), 2.04 (s, 1H), | |
| | | | 1.96 (d, J = 7.1 Hz, 3H), | |
| | | | 1.39 (s, 9H) | |
| 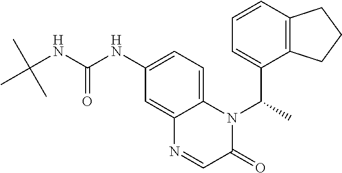 [SMILES: C[C@@H](c1cccc2c1CCC2)n1c(=O)cnc2cc(NC(=O)NC(C)(C)C)ccc21] | 125 | (S)-1-(tert-butyl)-3-(1- (1-(2,3-dihydro-1H- inden-4-yl)ethyl)-2- oxo-1,2- dihydroquinoxalin-6- yl)urea | ¹H NMR (400 MHz, DMSO-d₆) δ 8.38 (s, 1H), 8.23 (s, 1H),7.88- 7.98 (m, 1H), 7.40- 7.52 (m, 1H), 7.21- 7.38 (m, 3H), 7.10-7.20 (m, 1H), 6.48 (s, 1H), 6.00 (s, 1H), 2.59-2.85 (m, 3H), 1.72-1.98 (m, 6H), 1.22-1.38 (m, 9H). | calcd. for C₂₄H₂₈N₄O₂: 404.22, found: 405.10 [M + H]⁺ |
| 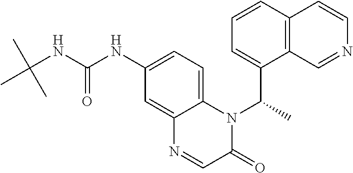 [SMILES: C[C@@H](c1cccc2ccncc12)n1c(=O)cnc2cc(NC(=O)NC(C)(C)C)ccc21] | 126 | (S)-1-(tert-butyl)-3-(1- (1-(isoquinolin-8- yl)ethyl)-2-oxo-1,2- dihydroquinoxalin-6- yl)urea | ¹H NMR (400 MHz, DMSO-d₆) δ 9.01 (s, 1H), 8.38-8.58 (m, 2H), 8.02-8.05 (m, 2H), 7.93- 8.02 (m, 1H), 7.76-7.93 (m, 3H), 7.50-7.76 (m, 1H), 7.21-7.76 (m, 1H), 6.85-7.20 (m, 1H), 5.78- 6.25 (m, 1H), 1.95-2.13 (m, 3H), 1.25-1.35 (m, 9H). | calcd. for C₂₄H₂₅N₅O₂: 415.2 m/z, found: 416.15 [M + H]⁺ |
| 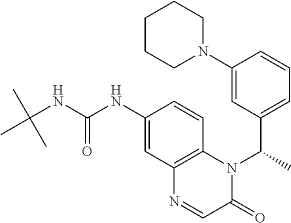 [SMILES: C[C@@H](c1cccc(N2CCCCC2)c1)n1c(=O)cnc2cc(NC(=O)NC(C)(C)C)ccc21] | 127 | (S)-1-(tert-butyl)-3-(2- oxo-1-(1-(3- (piperidin-1- yl)phenyl)ethyl)-1,2- dihydroquinoxalin-6- yl)urea | ¹H NMR (400 MHz, DMSO-d₆) δ 8.39 (s, 1H), 8.27 (s, 1H), 7.92 (d, J = 2.6 Hz, 1H), 7.26 (dd, J = 9.5, 2.5 Hz, 1H), 7.15 (t, J = 7.8 Hz, 1H), 7.10 (s, 1H), 6.81-6.83 (m, 2H), 6.65-6.59 (m, 1H), 6.58-6.53 (m, 1H), 6.03 (s, 1H), 3.05- 3.09 (m, 4H), 1.85 (d, J = 7.2 Hz, 3H), 1.48- 1.59 (m, 6H), 1.28 (s, 9H). | calcd. for C₂₆H₃₃N₅O₂: 447.28 m/z, found 448.25 [M + H]⁺ |
| 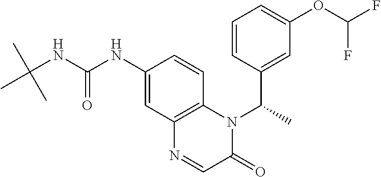 [SMILES: C[C@@H](c1cccc(OC(F)F)c1)n1c(=O)cnc2cc(NC(=O)NC(C)(C)C)ccc21] | 128 | (S)-1-(tert-butyl)-3-(1- (1-(3- (difluoromethoxy)phe- nyl)ethyl)-2-oxo-1,2- dihydroquinoxalin-6- yl)urea | ¹H NMR (400 MHz, DMSO-d₆) δ 8.41 (s, 1H), 8.26 (s, 1H),7.88- 8.00 (m, 1H), 7.21-7.50 (m, 3H), 7.00-7.17 (m, 4H), 6.49-6.67 (m, 1H), 6.03 (s, 1H), 1.88 (d, J = 7.1 Hz, 3H), 1.25-1.34 (s, 9H). ¹⁹F NMR (376 MHz, DMSO) δ −82.14. | LC/MS (ESI): calcd. for C₂₂H₂₄F₂N₄O₃: 430.2 m/z, found: 431.20 [M + H]⁺ |
| 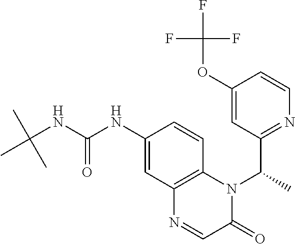 [SMILES: C[C@@H](c1cc(OC(F)(F)F)ccn1)n1c(=O)cnc2cc(NC(=O)NC(C)(C)C)ccc21] | 129 | (S)-1-(tert-butyl)-3-(2- oxo-1-(1-(4- (trifluoromethoxy)pyri- din-2-yl)ethyl)-1,2- dihydroquinoxalin-6- yl)urea | ¹H NMR (400 MHz, DMSO-d₆) δ 8.62 (d, J = 5.6 Hz, 1H), 8.43 (s, 1H), 8.21 (s, 1H), 7.94 (d, J = 2.5 Hz, 1H), 7.30- 7.48 (m, 3H), 7.15 (s, 1H), 6.51 (s, 1H), 6.04 (s, 1H), 1.89 (d, J = 7.0 Hz, 3H), 1.29 (s, 9H). ¹⁹F NMR (376 MHz, DMSO) δ (ppm): −56.5140. | calcd. for C₂₁H₂₂F₃N₅O₃: 449.17 m/z, found 450.05 [M + H]⁺ |
| 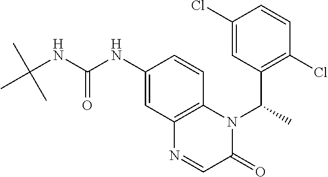 [SMILES: C[C@@H](c1cc(Cl)ccc1Cl)n1c(=O)cnc2cc(NC(=O)NC(C)(C)C)ccc21] | 130 | (S)-1-(tert-butyl)-3-(1- (1-(2,5- dichlorophenyl)ethyl)- 2-oxo-1,2- dihydroquinoxalin-6- yl)urea | ¹H NMR (400 MHz, DMSO-d₆) δ 8.46 (s, 1H), 8.08 (s, 1H), 7.83- 7.91 (m, 2H), 7.61 (s, 1H), 7.47-7.54 (m, 1H), 7.35-7.46 (m, 2H), 6.15 (s, 1H), 6.06 (s, 1H), 1.86 (d, J = 6.9 Hz, 3H), 1.30 (s, 9H). | calcd. for C₂₁H₂₂Cl₂N₄O₂: 43211 m/z, found: 432.95 [M + H]⁺ |
| 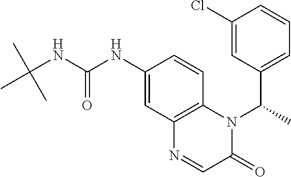 [SMILES: C[C@@H](c1cccc(Cl)c1)n1c(=O)cnc2cc(NC(=O)NC(C)(C)C)ccc21] | 131 | (S)-1-(tert-butyl)-3-(1- (1-(3- chlorophenyl)ethyl)-2- oxo-1,2- dihydroquinoxalin-6- yl)urea | ¹H NMR (400 MHz, DMSO-d₆) δ 8.42 (s, 1H), 8.25 (s, 1H), 7.90- 8.01 (m, 1H), 7.28-7.44 (m, 4H), 7.18-7.28 (m, 1H), 6.90-7.28 (m, 1H), 6.41-6.65 (m, 1H), 6.03 (s, 1H), 1.88 (d, J = 7.1 Hz, 3H), 1.29 (s, 9H). | calcd. for C₂₁H₂₃ClN₄O₂: 398.1 m/z, found: 399.15 [M + H]⁺ |
| 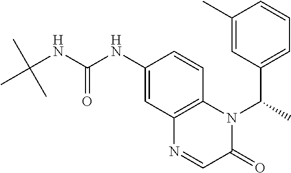 [SMILES: Cc1cccc([C@H](C)n2c(=O)cnc3cc(NC(=O)NC(C)(C)C)ccc32)c1] | 132 | (S)-1-(tert-butyl)-3-(2- oxo-1-(1-(m- tolyl)ethyl)-1,2- dihydroquinoxalin-6- yl)urea | ¹H NMR (400 MHz, DMSO-d₆) δ 8.38 (s, 1H), 8.27 (s, 1H), 7.93 (s, 1H), 7.23-7.25 (m, 2H), 7.07-7.10 (m, 4H), 6.57 (s, 1H), 6.03 (s, 1H), 2.27 (s, 3H), 1.85 (d, J = 7.1 Hz, 3H), 1.282 (s, 9H). | calcd. for C₂₂H₂₆N₄O₂: 378.21 m/z, found 379.25 [M +H]⁺ |
| 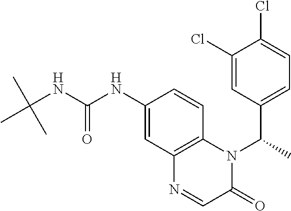 [SMILES: C[C@@H](c1ccc(Cl)c(Cl)c1)n1c(=O)cnc2cc(NC(=O)NC(C)(C)C)ccc21] | 133 | (S)-1-(tert-butyl)-3-(1- (1-(3,4- dichlorophenyl)ethyl)- 2-oxo-1,2- dihydroquinoxalin-6- yl)urea | ¹H NMR (400 MHz, DMSO-d₆) δ 8.43 (s, 1H), 8.09-8.29 (m, 1H), 7.93-7.98 (m, 1H), 7.55-7.62 (m, 2H), 6.82-7.47 (m, 3H), 6.52 (m, 1H), 6.04 (s, 1H), 1.87 (d, J = 7.0 Hz, 3H), 1.29 (s, 9H). | calcd. for C₂₁H₂₂Cl₂N₄O₂: 432.11 m/z, found:43 3.10 [M + H]⁺ |
| 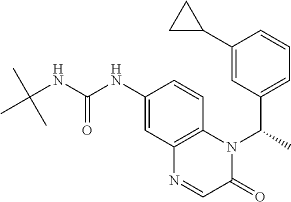 [SMILES: C[C@@H](c1cccc(C2CC2)c1)n1c(=O)cnc2cc(NC(=O)NC(C)(C)C)ccc21] | 134 | (S)-1-(tert-butyl)-3-(1- (1-(3- cyclopropylphenyl)eth- yl)-2-oxo-1,2- dihydroquinoxalin-6- yl)urea | ¹H NMR (400 MHz, Chloroform-d) δ 8.35 (s, 1H), 7.57 (d, J = 2.6 Hz, 1H), 7.50 (dd, J = 9.2, 2.6 Hz, 1H), 7.19 (td, J = 7.3, 1.3 Hz, 1H), 7.04 (d, J = 9.2 Hz, 1H), 7.00 (dt, J = 8.4, 1.1 Hz, 2H), 6.88 (d, J = 7.7 Hz, 1H), 6.79 (d, J = 7.5 Hz, 1H), 3.08 (s, 2H), 1.90 (d, J = 7.2 Hz, 3H), 1.82 (d, J = 8.5, 5.1 Hz, 1H), 1.36 (s, | calcd. for C₂₄H₂₈N₄O₂: 404.22 m/z found: 403.05 [M − H]⁻ |
| | | | 9H), 0.93 (dq, J = 8.4, | |
| | | | 1.8 Hz, 2H), 0.56 - 0.71 | |
| | | | (m, 2H) | |
| 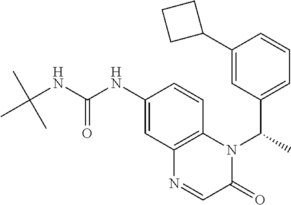 [SMILES: C[C@@H](c1cccc(C2CCC2)c1)n1c(=O)cnc2cc(NC(=O)NC(C)(C)C)ccc21] | 135 | (S)-1-(tert-butyl)-3-(1- (1-(3-cyclo- butylphenyl)ethyl)- 2-oxo-1,2- dihydroquinoxalin-6- yl)urea | ¹H NMR (400 MHz, DMSO-d₆) δ 8.40 (s, 1H), 8.27 (s, 1H), 7.92 (d, J = 2.5 Hz, 1H), 7.27 (t, J = 7.7 Hz, 2H), 7.00- 7.20 (m, 4H), 6.57 (s, 1H), 6.04 (s, 1H), 3.48- 3.75 (m, 1H), 2.18- 2.38 (m, 2H), 1.88- 2.12 (m, 6H), 1.70- 1.88 (m, 1H), 1.28 (s, 9H). | calcd. For C₂₅H₃₀N₄O₂: 418.24 m/z, found: 419.05 [M + H]⁺. |
| 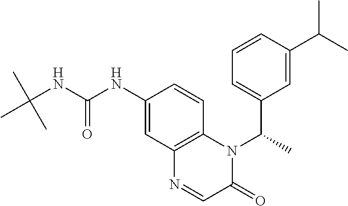 [SMILES: CC(C)c1cccc([C@H](C)n2c(=O)cnc3cc(NC(=O)NC(C)(C)C)ccc32)c1] | 136 | (S)-1-(tert-butyl)-3-(1- (1-(3-iso- propylphenyl)ethyl)- 2-oxo-1,2- dihydroquinoxalin-6- yl)urea | ¹H NMR (300 MHz, DMSO-d₆) δ 8.39 (s, 1H), 8.26 (s, 1H), 7.92 (d, J = 3.0 Hz, 1H), 7.20- 7.33 (m, 2H), 7.00- 7.19 (m, 4H), 6.55-6.62 (m, 1H), 6.02 (s, 1H), 2.77-2.95 (m, 1H), 1.88 (d, J = 6.0 Hz, 3H), 1.28 (s, 9H), 1.10-1.19 (m, 6H). | calcd. For C₂₄H₃₀N₄O₂: 406.2 m/z, found: 407.20 [M + H]⁺. |
| 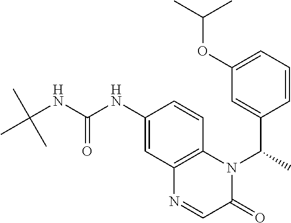 [SMILES: CC(C)Oc1cccc([C@H](C)n2c(=O)cnc3cc(NC(=O)NC(C)(C)C)ccc32)c1] | 137 | (S)-1-(tert-butyl)-3-(1- (1-(3- isopropoxyphenyl)eth- yl)-2-oxo-1,2- dihydroquinoxalin-6- yl)urea | ¹H NMR (400 MHz, DMSO-d₆) δ 8.41 (s, 1H), 8.26 (s, 1H), 7.85- 8.08 (m, 1H), 7.19-7.40 (m, 2H), 7.07 (s, 1H), 6.67-6.90 (m, 3H), 6.55 (s, 1H), 6.04 (s, 1H), 4.47-4.68 (m, 1H), 1.86 (d, J = 7.1 Hz, 3H), 1.11-1.38 (m, 15H). | calcd. for C₂₄H₃₀N₄O₃: 422.2 m/z, found: 423.20 [M + H]⁺ |
| 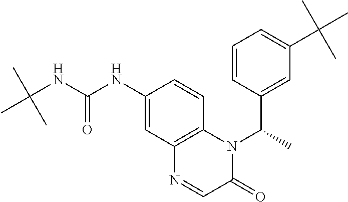 [SMILES: C[C@@H](c1cccc(C(C)(C)C)c1)n1c(=O)cnc2cc(NC(=O)NC(C)(C)C)ccc21] | 138 | (S)-1-(tert-butyl)-3-(1- (1-(3-(tert- butyl)phenyl)ethyl)-2- oxo-1,2- dihydroquinoxalin-6- yl)urea | ¹H NMR (400 MHz, DMSO-d₆) δ 8.41 (s, 1H), 8.27 (s, 1H), 7.81- 8.02 (m, 1H), 7.20-7.40 (m, 4H), 6.95-7.20 (s, 1H), 6.59 (s, 1H), 6.03 (s, 1H), 1.90 (d, J = 7.2 Hz, 3H), 1.21-1.31 (m, 18H). | calcd. for C₂₅H₃₂N₄O₂: 420.2 m/z, found: 421.25 [M + H]⁺. |
| 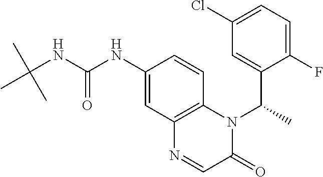 [SMILES: C[C@@H](c1cc(Cl)ccc1F)n1c(=O)cnc2cc(NC(=O)NC(C)(C)C)ccc21] | 139 | (S)-1-(tert-butyl)-3-(1- (1-(5-chloro-2- fluorophenyl)ethyl)-2- oxo-1,2- dihydroquinoxalin-6- yl)urea | ¹H NMR (400 MHz, DMSO-d₆) δ 8.45 (s, 1H), 8.14 (s, 1H), 7.90 (m, 1H), 7.73 (m, 1H), 7.37-7.56 (m, 3H), 7.09- 7.19 (m, 1H), 6.47 (m, 1H), 6.04 (s, 1H), 1.87 (d, J = 7.0 Hz, 3H), 1.30 (s, 9H). ¹⁹F NMR (376 MHz, DMSO) δ −118.23. | calcd. for C₂₁H₂₂ClFN₄O₂: 416.14 m/z, found: 417.10 [M + H]⁺ |
| 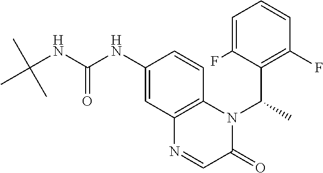 [SMILES: C[C@@H](c1c(F)cccc1F)n1c(=O)cnc2cc(NC(=O)NC(C)(C)C)ccc21] | 140 | (S)-1-(tert-butyl)-3-(1- (1-(2,6- difluorophenyl)ethyl)- 2-oxo-1,2- dihydroquinoxalin-6- yl)urea | ¹H NMR (300 MHz, DMSO-d₆) δ 8.46 (s, 1H), 8.11 (s, 1H), 7.91 (d, J = 2.5 Hz, 1H), 7.61-7.79 (m, 1H), 7.49- 7.61 (m, 1H), 7.29-7.49 (m, 1H), 6.96-7.10 (m, 2H), 6.39-6.59 (m, 1H), 6.05 (s, 1H),1.77-1.99 (m, 3H), 1.31 (s, 9H). ¹⁹F NMR (282 MHz, | calcd. for C₂₁H₂₂F₂N₄O₂: 400.17 m/z, found: 401.20 [M + H]⁺ |
| | | | DMSO) 8 -110.22. | |
| 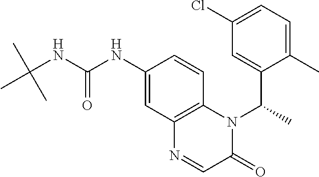 [SMILES: Cc1ccc(Cl)cc1[C@H](C)n1c(=O)cnc2cc(NC(=O)NC(C)(C)C)ccc21] | 141 | (S)-1-(tert-butyl)-3-(1- (1-(5-chloro-2- methylphenyl)ethyl)- 2-oxo-1,2- dihydroquinoxalin-6- yl)urea | ¹H NMR (300 MHz, DMSO-d₆) δ 8.43 (s, 1H), 8.14 (s, 1H), 7.88 (d, J = 2.3 Hz, 1H), 7.66- 7.72 (m, 1H), 7.36- 7.54 (m, 2H), 7.22- 7.30 (m, 1H), 7.11 (d, J = 8.2 Hz, 1H), 6.18- 6.34 (m, 1H), 6.05 (s, 1H), 1.86 (d, J = 6.9 Hz, 3H), 1.80 (s, 3H), 1.29 | calcd. for C₂₂H₂₅ClN₄O₂: 412.17 m/z, found: 413.20 [M + H]⁺ |
| | | | (s, 9H). | |
| 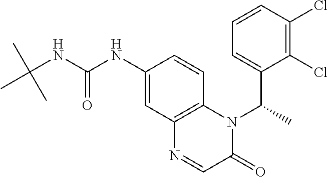 [SMILES: C[C@@H](c1cccc(Cl)c1Cl)n1c(=O)cnc2cc(NC(=O)NC(C)(C)C)ccc21] | 142 | (S)-1-(tert-butyl)-3-(1- (1-(2,3- dichlorophenyl)ethyl)- 2-oxo-1,2- dihydroquinoxalin-6- yl)urea | ¹H NMR (400 MHz, DMSO-d₆) δ 8.45 (s, 1H), 8.10 (s, 1H), 7.89 (d, J = 2.5 Hz, 1H), 7.81- 7.87 (m, 1H), 7.62 (dd, J = 8.0, 1.4 Hz, 1H), 7.53-7.58 (m, 1H), 7.47 (t, J = 8.0 Hz, 2H), 6.22 (s, 1H), 6.05 (s, 1H), 1.88 (d, J = 6.9 Hz, 3H), 1.30 (s, 9H). | calcd. for C₂₁H₂₂Cl₂N₄O₂: 432.11 m/z, found 433.10 [M + H]⁺ |
| 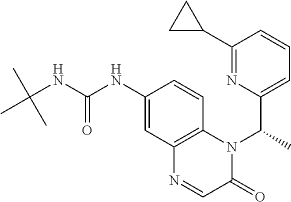 [SMILES: C[C@@H](c1cccc(C2CC2)n1)n1c(=O)cnc2cc(NC(=O)NC(C)(C)C)ccc21] | 143 | (S)-1-(tert-butyl)-3-(1- (1-(6- cyclopropylpyridin-2- yl)ethyl)-2-oxo-1,2- dihydroquinoxalin-6- yl)urea | ¹H NMR (300 MHz, DMSO-d₆) δ 8.40 (s, 1H), 8.24 (s, 1H), 7.90 (d, J = 2.5 Hz, 1H), 7.58 (t, J = 7.7 Hz, 1H), 7.31 (dd, J = 9.3, 2.5 Hz, 1H), 7.16 (d, J = 7.7 Hz, 1H), 7.05 (dd, J = 7.8, 0.9 Hz, 2H), 6.50 (s, 1H), 6.03 (s, 1H), 2.03 (tt, J = 7.6, 5.0 Hz, 1H), 1.78-1.95 (m, 3H), 1.20-1.40 (m, 9H), 0.75-0.95 (m, | calcd. for C₂₃H₂₇N₅O₂: 405.22 m/z. found 406.20 [M + H]⁺ |
| | | | 3H), 0.53-0.70 (m, | |
| | | | 1H). | |
| 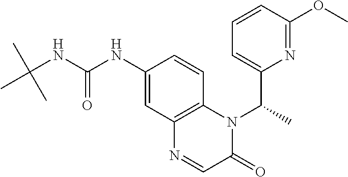 [SMILES: COc1cccc([C@H](C)n2c(=O)cnc3cc(NC(=O)NC(C)(C)C)ccc32)n1] | 144 | (S)-1-(tert-butyl)-3-(1- (1-(6-methoxypyridin- 2-yl)ethyl)-2-oxo-1,2- dihydroquinoxalin-6- yl)urea | ¹H NMR (300 MHz, DMSO-d₆) δ 8.40 (s, 1H), 8.25 (s, 1H), 7.93 (d, J = 3.0 Hz, 1H), 7.60- 7.72 (m, 1H), 7.34- 7.39 (m, 1H), 7.12 (s, 1H), 6.91 (d, J = 9.0 Hz, 1H), 6.70 (d, J = 9.0 Hz, 1H), 6.52 (s, 1H), 6.03 (s, 1H), 3.68 (s, 3H), 1.90 (d, J = 9.0 Hz, 3H), | calcd. For C₂₁H₂₅N₅O₃: 395.2 m/z, found: 396.15 [M + H]⁺, |
| | | | 1.29 (s, 9H). | |
| 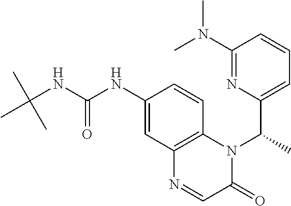 [SMILES: C[C@@H](c1cccc(N(C)C)n1)n1c(=O)cnc2cc(NC(=O)NC(C)(C)C)ccc21] | 145 | (S)-1-(tert-butyl)-3-(1- (1-(6-(dimethyl- amino)pyridin- 2-yl)ethyl)-2-oxo- 1,2- dihydroquinoxalin-6- yl)urea | ¹H NMR (400 MHz, DMSO-d₆) δ 8.38 (s, 1H), 8.25 (s, 1H), 7.90 (d, J = 2.5 Hz, 1H), 7.43- 7.54 (m, 1H), 7.26- 7.33 (m, 1H), 7.13 (s, 1H), 6.43-6.52 (m, 3H), 6.03 (s, 1H), 2.92 (s, 6H), 1.86 (d, J = 7.2 Hz, 3H), 1.29 (s, 9H). | calcd. for C₂₀H₂₁FN₄O₂, 408.23 m/z, found 409.20 [M + H]⁺ |
| 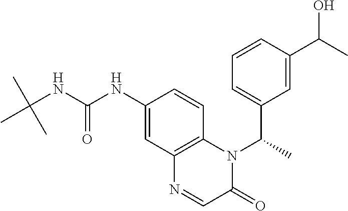 [SMILES: CC(O)c1cccc([C@H](C)n2c(=O)cnc3cc(NC(=O)NC(C)(C)C)ccc32)c1] | 146 | 1-(tert-butyl)-3-(1- ((1S)-1-(3-(1-hydroxy- ethyl)phenyl)eth- yl)-2-oxo-1,2- dihydroquinoxalin-6- yl)urea | ¹H NMR (300 MHz, DMSO-d₆) δ 8.38 (s, 1H), 8.27 (s, 1H), 7.91 (d, J = 2.5 Hz, 1H), 7.18- 7.36 (m, 4H), 6.98- 7.18 (m, 2H), 6.59 (s, 1H), 6.01 (s, 1H), 5.13 (dd, J = 4.3, 3.4 Hz, 1H), 4.67 (q, J = 5.6 Hz, 1H), 1.88 (d, J = 7.1 Hz, 3H), 1.21-1.31 (m, 12H). | calcd. for C₂₃H₂₈N₄O₃: 408.22 m/z, found 409.25 [M + H]⁺ |
| 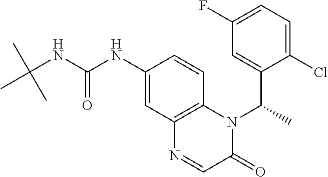 [SMILES: C[C@@H](c1cc(F)ccc1Cl)n1c(=O)cnc2cc(NC(=O)NC(C)(C)C)ccc21] | 147 | (S)-1-(tert-butyl)-3-(1- (1-(2-chloro-5- fluorophenyl)ethyl)-2- oxo-1,2- dihydroquinoxalin-6- yl)urea | ¹H NMR (400 MHz, DMSO-d₆) δ 8.45 (s, 1H), 8.09 (s, 1H), 7.89 (d, J =2.5 Hz, 1H), 7.67-7.80(m, 1H),7.58 (s, 1H),7.47(d, J = 8.0 z, 1H), 7.30-7.45 (m, 1H), 6.17 (s, 1H), 6.05 (s, 1H), 1.86(d, J = 6.90 Hz, 3H), 1.30 (s, 9H); ¹⁹F NMR (376 MHz, | calcd. for C₂₁H₂₂ClFN₄O₂: 416.14, found: 417.20 [M + H]⁺ |
| | | | DMSO) δ −115.01 | |
| 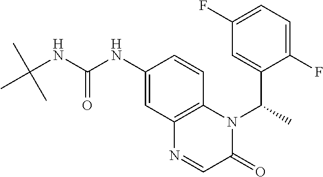 [SMILES: C[C@@H](c1cc(F)ccc1F)n1c(=O)cnc2cc(NC(=O)NC(C)(C)C)ccc21] | 148 | (S)-1-(tert-butyl)-3-(1- (1-(2,5- difluorophenyl)ethyl)- 2-oxo-1,2- dihydroquinoxalin-6- yl)urea | ¹H NMR (400 MHz, DMSO-d₆) δ 8.44 (s, 1H), 8.15 (s, 1H), 7.90 (d, J = 2.4 Hz, 1H), 7.31-7.67(m, 3H), 7.00- 7.28 (m, 2H), 8.15 (s, 1H), 6.04 (s, 1H), 1.86 (d, J = 7.1 Hz, 3H), 1.30 (s, 9H); ¹⁹F NMR (376 MHz, DMSO) δ −118.38, −121.67 | calcd. for C₂₁H₂₂F₂N₄O₂: 400.17, found: 401.20 [M + H]⁺ |
| 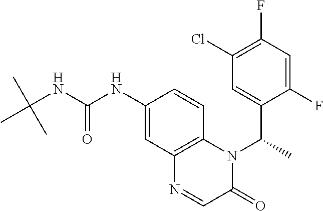 [SMILES: C[C@@H](c1cc(Cl)c(F)cc1F)n1c(=O)cnc2cc(NC(=O)NC(C)(C)C)ccc21] | 149 | (S)-1-(tert-butyl)-3-(1- (1-(5-chloro-2,4- difluorophenyl)ethyl)- 2-oxo-1,2- dihydroquinoxalin-6- yl)urea | ¹H NMR (300 MHz, DMSO-d₆) δ 8.47 (s, 1H), 8.12 (s, 1H), 7.85- 8.05 (m, 2H), 7.30- 7.79 (m, 3H), 6.43 (s, 1H), 6.05 (s, 1H), 1.86 (d, J = 7.0 Hz, 3H), 1.25- 1.40 (m, 9H). ¹⁹F NMR (282 MHz, DMSO) δ −112.655, −112.996. | calcd. for C₂₁H₂₁ClF₂N₄O₂: 434.13 m/z, found 435.13 [M + H]⁺ |
| 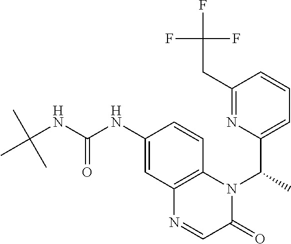 [SMILES: C[C@@H](c1cccc(CC(F)(F)F)n1)n1c(=O)cnc2cc(NC(=O)NC(C)(C)C)ccc21] | 150 | (S)-1-(tert-butyl)-3-(2- oxo-1-(1-(6-(2,2,2- trifluoroethyl)pyridin- 2-yl)ethyl)-1,2- dihydroquinoxalin-6- yl)urea | ¹H NMR (300 MHz, DMSO-d₆) δ 8.41 (s, 1H), 8.24 (s, 1H), 7.84- 7.97 (m, 1H), 7.66-7.84 (m, 1H), 7.33 (dd, J = 20.3, 7.8 Hz, 3H), 7.15 (s, 1H), 6.59 (s, 1H), 6.00 (s, 1H), 3.70-3.90 (m, 2H), 1.89 (d, J = 7.1 Hz, 3H), 1.28 (s, 9H). ¹⁹F NMR (282 MHz, DMSO) δ −63.10. | calcd. for C₂₂H₂₄F₃N₅O₂: 447.1 m/z, found: 448.20 [M + H]⁺ |
| 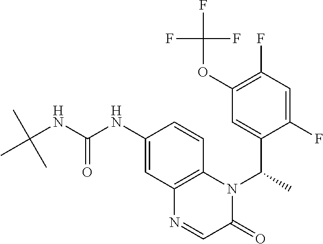 [SMILES: C[C@@H](c1cc(OC(F)(F)F)c(F)cc1F)n1c(=O)cnc2cc(NC(=O)NC(C)(C)C)ccc21] | 151 | (S)-1-(tert-butyl)-3-(1- (1-(2,4-difluoro-5- (trifluoromethoxy)phe- nyl)ethyl)-2-oxo-1,2- dihydroquinoxalin-6- yl)urea | ¹H NMR (400 MHz, DMSO-d₆) δ 8.48 (s, 1H), 8.12 (s, 1H), 7.88- 7.95 (m, 2H), 7.61 (s, 1H), 7.52 (q, J = 10.3 Hz, 2H), 6.44 (s, 1H), 6.04 (s, 1H), 1.86 (d, J = 7.0 Hz, 3H), 1.30 (s, 9H). ¹⁹F NMR (376 MHz, DMSO) δ (ppm): −58.2434, −111.0659, −126.3474. | calcd. for C₂₂H₂₁F₅N₄O₃: 484.15 m/z, found 485.15 [M + H]⁺ |
| 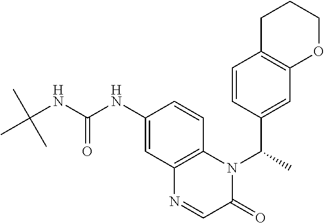 [SMILES: C[C@@H](c1ccc2c(c1)OCCC2)n1c(=O)cnc2cc(NC(=O)NC(C)(C)C)ccc21] | 152 | (S)-1-(tert-butyl)-3-(1- (1-(chroman-7- yl)ethyl)-2-oxo-1,2- dihydroquinoxalin-6- yl)urea | ¹H NMR (400 MHz, DMSO-d₆) δ 8.39 (s, 1H), 8.25 (s, 1H), 7.91 (d, J = 2.6 Hz, 1H), 7.25- 7.32 (m, 1H), 7.08 (s, 1H), 7.00 (d, J = 7.9 Hz, 1H), 6.67 (dd, J = 8.1, 2.1 Hz, 1H), 6.62 (t, J = 1.4 Hz, 1H), 6.51 (s, 1H), 6.03 (s, 1H), 4.10 (td, J = 6.6, 5.9, 3.5 Hz, 2H), 2.70 (d, J = 6.5 Hz, 2H), 1.89 (td, J = 6.5, 6.1, 3.7 Hz, 2H), 1.83 (d, | calcd. for C₂₄H₂₈N₄O₃: 420.22 m/z, found 421.15 [M + H]⁺ |
| | | | J = 7.2 Hz, 3H), 1.28 (s, | |
| | | | 9H). | |
| 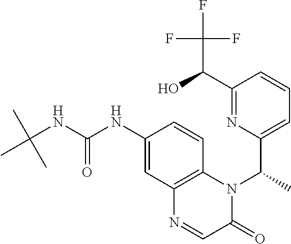 [SMILES: C[C@@H](c1cccc([C@@H](O)C(F)(F)F)n1)n1c(=O)cnc2cc(NC(=O)NC(C)(C)C)ccc21] | 153 | 1-(tert-butyl)-3-(2- oxo-1-((S)-1-(6-((S)- 2,2,2-trifluoro-1- hydroxyethyl)pyridin- 2-yl)ethyl)-1,2- dihydroquinoxalin-6- yl)urea | ¹H NMR (400 MHz, Acetonitrile-d₃) δ 8.22 (s, 1H), 7.80-8.00 (m, 2H), 7.46 (d, J = 7.7 Hz, 1H), 7.38 (d, J = 7.5 Hz, 2H), 7.09 (d, J = 13.0 Hz, 2H), 6.72 (s, 1H), 5.10-5.20 (s, 1H), 5.00 (s, 1H), 1.2-1.40 (m, 12H). ¹⁹F NMR (376 MHz, CD₃CN) δ −78.28. | calcd. For C₂₂H₂₄F₃N₅O₃: 463.18 m/z, found: 464.20 [M + H]⁺. |
| 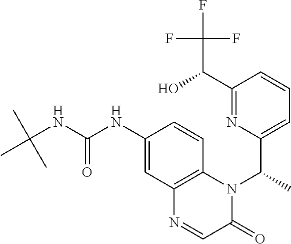 [SMILES: C[C@@H](c1cccc([C@H](O)C(F)(F)F)n1)n1c(=O)cnc2cc(NC(=O)NC(C)(C)C)ccc21] | 154 | 1-(tert-butyl)-3-(2- oxo-1-((S)-1-(6-((R)- 2,2,2-trifluoro-1- hydroxyethyl)pyridin- 2-yl)ethyl)-1,2- dihydroquinoxalin-6- yl)urea | ¹H NMR (400 MHz, Acetonitrile-d₃) δ 8.21 (s, 1H), 7.85-7.76 (m, 2H), 7.47 (d, J = 7.7 Hz, 1H), 7.32-7.40 (m, 2H), 7.17 (s, 1H), 7.06 (d, J = 9.2 Hz, 1H), 6.70 (s, 1H), 5.13 - 5.25 (m, 2H), 5.05 (s, 1H), 1.97- 1.99 (m, 3H), 1.34 (s, 9H). ¹⁹F NMR (376 MHz, CD₃CN) δ −78.24. | calcd. For C₂₂H₂₄F₃N₅O₃: 463.18 m/z, found: 464.15 [M + H]⁺. |
| 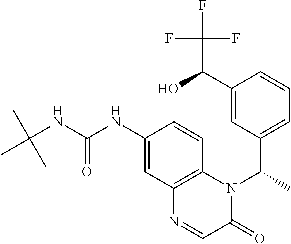 [SMILES: C[C@@H](c1cccc([C@@H](O)C(F)(F)F)c1)n1c(=O)cnc2cc(NC(=O)NC(C)(C)C)ccc21] | 155 | 1-(tert-butyl)-3-(2- oxo-1-((S)-1-(3-((S)- 2,2,2-trifluoro-1- hydroxyethyl)phe- nyl)ethyl)-1,2- dihydroquinoxalin-6- yl)urea | ¹H NMR (400 MHz, Acetonitrile-d₃) δ 8.23 (s, 1H), 7.80 (d, J = 2.6 Hz, 1H), 7.35-7.46 (m, 4H), 7.30-7.38 (m, 1H), 7.12 (s, 1H), 7.01 (d, J = 9.2 Hz, 1H), 6.70 (s, 1H), 5.17 (s, 1H), 4.99-5.27 (m, 1H), 4.61 (d, J = 5.4 Hz, 1H), 1.95-1.98 (m, 3H), 1.34 (s, 9H). ¹⁹F NMR (376 MHz, CD₃CN) δ −78.78. | calcd. For C₂₃H₂₅F₃N₄O₃: 462.19 m/z, found: 463.15 [M + H]⁺. |
| 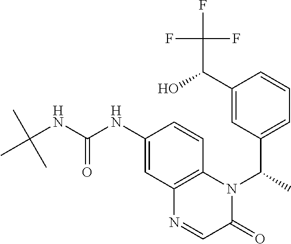 [SMILES: C[C@@H](c1cccc([C@H](O)C(F)(F)F)c1)n1c(=O)cnc2cc(NC(=O)NC(C)(C)C)ccc21] | 156 | 1-(tert-butyl)-3-(2- oxo-1-((S)-1-(3-((R)- 2,2,2-trifluoro-1- hydroxyethyl)phe- nyl)ethyl)-1,2- dihydroquinoxalin-6- yl)urea | ¹H NMR (400 MHz, Acetonitrile-d₃) δ 8.23 (s, 1H), 7.83 (d, J = 2.6 Hz, 1H), 7.30-7.50 (m, 5H), 7.12 (s, 1H), 7.04 (d, J = 9.0 Hz, 1H), 6.70 (s, 1H), 5.15 (s, 1H), 5.02-5.13 (m, 1H), 4.56 (d, J = 5.7 Hz, 1H), 1.95-1.98 (m, 3H), 1.34 (s, 9H). ¹⁹F NMR (376 MHz, CD₃CN) δ −78.78. | calcd. For C₂₃H₂₅F₃N₄O₃: 462.19 m/z, found: 463.15 [M + H]⁺. |

### Example 157: 3-tert-butyl-1-[(2R)-2-methyl-4-[(3-methylphenyl)methyl]-3-oxo-2H-1,4-benzoxazin-7-yl]urea

### Synthetic Scheme

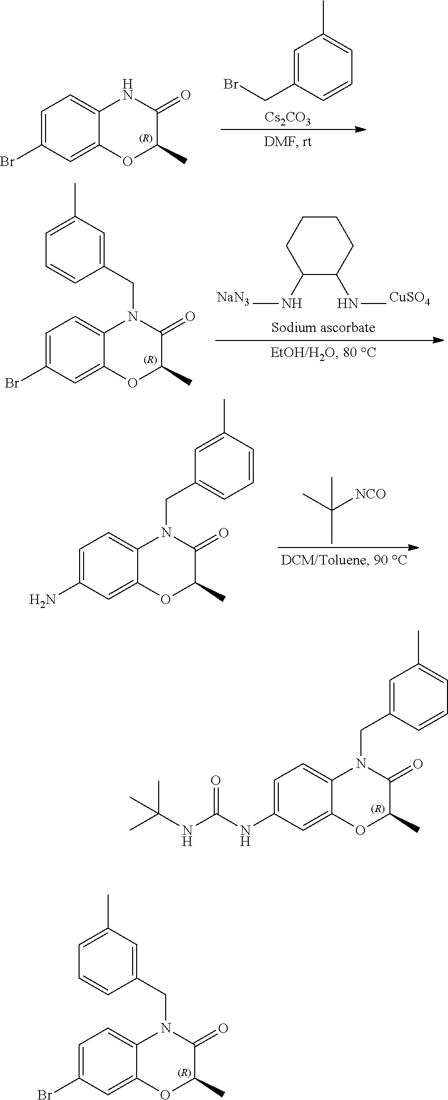

### (2R)-7-bromo-2-methyl-4-[(3-methylphenyl)methyl]-2H-1,4-benzoxazin-3-one

To a stirred solution of (2R)-7-bromo-2-methyl-2,4-dihydro-1,4-benzoxazin-3-one (400 mg, 1.652 mmol, 1 equiv) in DMF (4 mL) was added 1-(bromomethyl)-3-methylbenzene (458.70 mg, 2.478 mmol, 1.5 equiv) and Cs₂CO₃ (1080.10 mg, 3.304 mmol, 2 equiv) in portions at room temperature under nitrogen atmosphere. The resulting mixture was stirred for 3 h at room temperature under nitrogen atmosphere. The reaction was monitored by LCMS. Quenched with water (20 mL) and extracted with EA (3×20 mL). The combined extracts were washed with water, brine, dried over Na₂SO₄, filtered and concentrated. The residue was purified by silica gel column chromatography, eluted with PE/EA (0-100%) to afford (2R)-7-bromo-2-methyl-4-[(3-methylphenyl)methyl]-2H-1,4-benzoxazin-3-one (500 mg, 87.40%) as a white solid. LC/MS(ESI): mass calcd. for C₁₇H₁₆BrNO₂: 345.04 m/z, found 346.00 [M+H, M+H+2]⁺.

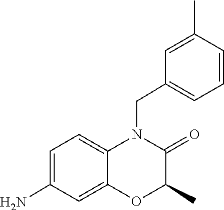 [SMILES: Cc1cccc(CN2C(=O)[C@@H](C)Oc3cc(N)ccc32)c1]

### (2R)-7-amino-2-methyl-4-[(3-methylphenyl)methyl]-2H-1,4-benzoxazin-3-one

To a stirred solution of (2R)-7-bromo-2-methyl-4-[(3-methylphenyl)methyl]-2H-1,4-benzoxazin-3-one (490 mg, 1.192 mmol, 1 equiv) in EtOH/H₂O (4:1, 5 mL) was added N₁,N₂-dimethylcyclohexane-1,2-diamine (101.69 mg, 0.715 mmol, 0.6 equiv), CuSO₄ (190.17 mg, 1.192 mmol, 1 equiv), sodium ascorbate (474.50 mg, 2.384 mmol, 2 equiv) and NaN₃ (232.39 mg, 3.576 mmol, 3 equiv) in portions at room temperature under nitrogen atmosphere. The resulting mixture was stirred for 3 h at 80° C. under nitrogen atmosphere. The reaction was monitored by LCMS. The mixture was allowed to cool down to room temperature. Quenched with water (30 mL) and extracted with EA (3×30 mL). The combined extracts were washed with water, brine, dried over Na₂SO₄, filtered and concentrated. The residue was purified by silica gel column chromatography, eluted with PE/EA (0-100%) to afford (2R)-7-amino-2-methyl-4-[(3-methylphenyl)methyl]-2H-1,4-benzoxazin-3-one (150 mg, 44.59%) as a white solid. LC/MS(ESI): mass calcd. for C₉H₁₈N₂O₃: 282.14 m/z, found 283.10 [M+H]⁺.

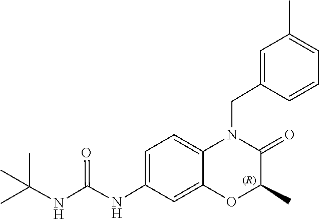

### 3-tert-butyl-1-[(2R)-2-methyl-4-[(3-methylphenyl)methyl]-3-oxo-2H-1,4-benzoxazin-7-yl]urea

To a stirred solution of (2R)-7-amino-2-methyl-4-[(3-methylphenyl)methyl]-2H-1,4-benzoxazin-3-one (140 mg, 0.496 mmol, 1 equiv) in DCM/Toluene (2:1, 2 mL) was added 2-isocyanato-2-methylpropane (147.47 mg, 1.488 mmol, 3 equiv) dropwise at room temperature under nitrogen atmosphere. The resulting mixture was stirred overnight at 90° C. under nitrogen atmosphere. The reaction was monitored by LCMS. The mixture was allowed to cool down to room temperature. Quenched with water (10 mL) and extracted with DCM (3×10 mL). The combined extracts were washed with water, brine, dried over Na₂SO₄, filtered and concentrated. The crude product was purified by Prep-PLC using a X Bridge Prep OBD C18 150 mm×30 mm×5 m column (eluent: 1700 to 42% (v/v) CH₃CN and H₂O with 10 mmol/L NH₄HCO₃) to afford 3-tert-butyl-1-[(2R)-2-methyl-4-[(3-methylphenyl)methyl]-3-oxo-2H-1,4-benzoxazin-7-yl]urea (41.1 mg, 21.37%) as a white solid. LC/MS(ESI): mass calcd. for C₂₂H₂₇N₃O₃: 381.21 m/z, found 382.15 [M+H]⁺. ¹H NMR (300 MHz, DMSO-d₆) δ 8.19 (s, 1H), 7.23-7.18 (m, 2H), 7.06-6.99 (m, 3H), 6.84 (d, J=8.7 Hz, 1H), 6.76-6.72 (m, 1H), 5.93 (s, 1H), 5.05 (m, 2H), 4.84-4.78 (m, 1H), 2.26 (s, 3H), 1.48 (d, J=6.6 Hz, 3H), 1.26 (s, 9H).

The following examples in Table 7 were made according to the procedure for example 157.

TABLE 7

| | | IUPAC | | |
|---|---|---|---|---|
| Structure | Ex. | Name | 1H NMR | LCMS |
| 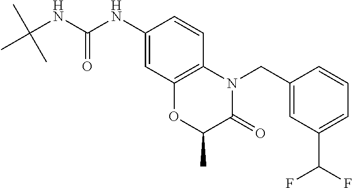 [SMILES: C[C@H]1Oc2cc(NC(=O)NC(C)(C)C)ccc2N(Cc2cccc(C(F)F)c2)C1=O] | 158 | (R)-1-(tert- butyl)- 3-(4-(3- (difluoro- methyl) benzyl)-2- methyl-3- oxo-3,4- dihydro- 2H- benzo [b][1,4] oxazin- 7-yl)urea | ¹H NMR (300 MHz, DMSO-d₆) δ 8.17- 8.30 (m, 1H), 7.34- 7.55 (m, 4H), 6.97- 7.30 (m, 2H), 6.80- 6.91 (m, 1H), 6.68- 6.91 (m, 1H), 5.86- 6.01 (m, 1H), 5.08- 5.27 (m, 2H), 4.73- 4.99 (m, 1H), 1.44- 1.59 (m, 3H), 1.20- 1.37 (m, 9H). ¹⁹F NMR (282 MHz, DMSO) δ (ppm): −109.702, −109.742. | calcd. for C₂₂H₂₅F₂N₃O₃: 417.19 m/z, found 418.15 [M + H]⁺ |
| 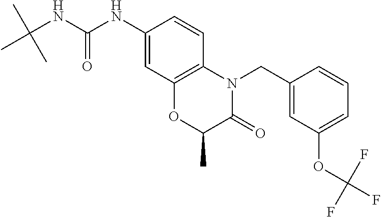 [SMILES: C[C@H]1Oc2cc(NC(=O)NC(C)(C)C)ccc2N(Cc2cccc(OC(F)(F)F)c2)C1=O] | 159 | (R)-1- (tert-butyl)- 3-(2- methyl-3- oxo-4-(3- (trifluoro- methoxy) benzyl)-3,4- dihydro-2H- benzo [b][1,4] oxazin-7- yl)urea | ¹H NMR (400 MHz, DMSO-d₆) δ 8.23 (s, 1H), 7.48 (m, 1H), 7.22-7.29 (m, 4H), 6.77-6.88 (m, 2H), 5.95 (s, 1H), 5.14 (m, 2H), 4.84 (m, 1H), 1.48 (m, 3H), 1.26 (s, 9H). ¹⁹F NMR (376 MHz, DMSO) δ −56.70. | calcd. for C₂₂H₂₄N₃O₄: 451.17 m/z, found 452.05 [M + H]+ |
| 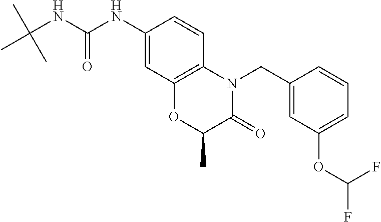 [SMILES: C[C@H]1Oc2cc(NC(=O)NC(C)(C)C)ccc2N(Cc2cccc(OC(F)F)c2)C1=O] | 160 | (R)-1- (tert-butyl)- 3-(4-(3- (difluoro- methoxy) benzyl)-2- methyl- 3-oxo-3,4- dihydro-2H- benzo [b][1,4] oxazin-7- yl)urea | ¹H NMR (400 MHz, DMSO-d₆) δ 8.23 (s, 1H), 7.41 (m, 1H), 7.23-7.06 (m, 5H), 6.86 (d, J = 8.8 Hz, 1H), 6.78 (m, 1H), 5.95 (s, 1H), 5.11- 5.10 (m, 2H), 4.83 (q, J = 6.8 Hz, 1H), 1.48 (d, J = 6.8 Hz, 3H), 1.26 (s, 9H). | calcd. for C₂₂H₂₅F₂N₃O₄: 433.2 m/z, found:434.15 [M + H]⁺ |
| 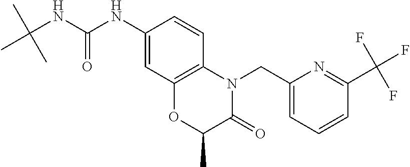 [SMILES: C[C@H]1Oc2cc(NC(=O)NC(C)(C)C)ccc2N(Cc2cccc(C(F)(F)F)n2)C1=O] | 161 | (R)-1- (tert-butyl)- 3-(2- methyl-3- oxo-4-((6- (trifluoro- methyl) pyridin-2- yl)methyl)- 3,4- dihydro-2H- benzo [b][1,4] oxazin-7- yl)urea | ¹H NMR (300 MHz, DMSO-d₆) δ 8.16- 8.30 (m, 1H), 7.96- 8.16 (m, 1H), 7.76- 6.90 (m, 1H), 7.43- 7.60 (m, 1H), 7.15- 6.29 (m, 1H), 6.70- 6.86 (m, 2H), 5.90- 6.04 (m, 1H), 5.12- 5.30 (m, 2H), 4.75- 4.90 (m, 1H), 1.35- 1.50 (m, 3H), 1.15- 1.35 (m, 9H). ¹⁹F NMR (282 MHz, DMSO) δ (ppm): −66.548. | calcd. for C₂₁H₂₃F₃N₄O₃: 436.17 m/z, found 437.15 [M + H]⁺ |
| 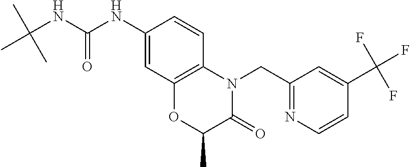 [SMILES: C[C@H]1Oc2cc(NC(=O)NC(C)(C)C)ccc2N(Cc2cc(C(F)(F)F)ccn2)C1=O] | 162 | (R)-1- (tert-butyl)- 3-(2- methyl-3- oxo-4-((4- (trifluoro- methyl) pyridin-2- yl)methyl)- 3,4- dihydro-2H- benzo [b][1,4] oxazin-7- yl)urea | ¹H NMR (400 MHz, DMSO-d₆) δ 8.55- 8.98 (m, 1H), 8.09- 8.35 (m, 1H), 7.53- 7.84 (m, 2H), 7.14- 7.40 (m, 1H), 6.82- 6.98 (m, 1H), 6.62- 6.80 (m, 1H), 5.76- 6.10 (m, 1H), 5.13- 5.45 (m, 2H), 4.60- 4.92 (m, 1H), 1.40- 1.55 (m, 3H), 1.20- 1.37 (m, 9H). ¹⁹F NMR (376 MHz, DMSO) δ (ppm): −63.3996. | calcd. for C₂₁H₂₃F₃N₄O₃: 436.17 m/z, found 437.15 [M + H]⁺ |
| 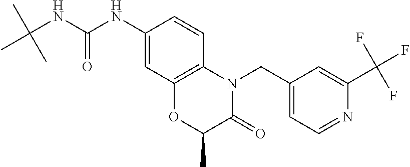 [SMILES: C[C@H]1Oc2cc(NC(=O)NC(C)(C)C)ccc2N(Cc2ccnc(C(F)(F)F)c2)C1=O] | 163 | (R)-1- (tert-butyl)- 3-(2- methyl-3- oxo-4-((2- (trifluoro- methyl) pyridin-4- yl)methyl)- 3,4- dihydro-2H- benzo [b][1,4] oxazin-7- yl)urea | ¹H-NMR (300 MHz, DMSO-d₆) δ 8.70 (d, J = 5.0 Hz, 1H), 8.24 (s, 1H), 7.81 (d, J = 1.6 Hz, 1H), 7.43-7.51 (m, 1H), 7.27 (d, J = 2.0 Hz, 1H), 6.72- 6.86 (m, 2H), 5.95 (s, 1H), 5.13-5.36 (m, 1H), 4.83-4.95 (m, 1H), 1.50 (d, J = 6.8 Hz, 3H), 1.27 (s, 9H). ¹⁹F-NMR (282 MHz, DMSO) δ −66.54. | calcd. for C₂₁H₂₃F₃N₄O₃: 436.17 m/z, found: 437.00 [M + H]⁺. |
| 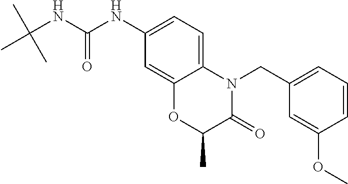 [SMILES: COc1cccc(CN2C(=O)[C@@H](C)Oc3cc(NC(=O)NC(C)(C)C)ccc32)c1] | 164 | (R)-1- (tert-butyl)- 3-(4-(3- methoxy- benzyl)- 2-methyl- 3-oxo- 3,4-dihydro- 2H- benzo [b][1,4] oxazin-7- yl)urea | ¹H NMR (300 MHz, DMSO-d₆) δ 8.20 (s, 1H), 7.18-7.29 (m, 2H), 6.70-6.89 (m, 5H), 5.93 (s, 1H), 5.05 (s, 2H), 4.79-4.82 (m, 1H), 3.71 (s, 3H), 1.47 (s, 3H), 1.26 (s, 9H). | calcd. for C₂₂H₂₇N₃O₄: 397.20 m/z, found: 398.10 [M + H]⁺. |
| 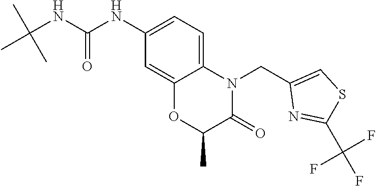 [SMILES: C[C@H]1Oc2cc(NC(=O)NC(C)(C)C)ccc2N(Cc2csc(C(F)(F)F)n2)C1=O] | 165 | (R)-1- (tert-butyl)- 3-(2- methyl-3- oxo-4-((2- (trifluoro- methyl) thiazol-4- yl)methyl)- 3,4- dihydro- 2H- benzo [b][1,4] oxazin-7- yl)urea | ¹H NMR (300 MHz, DMSO-d₆) δ 8.23 (s, 1H), 7.89 (s, 1H), 7.22 (d, J = 2.1 Hz, 1H), 6.99 (d, J = 8.7 Hz, 1H), 6.82 (d, J = 8.7 Hz, 1H), 5.95 (s, 1H), 5.25-5.16 (m, 2H), 4.79-4.77 (m, 1H), 1.45 (d, J = 6.6 Hz, 3H), 1.27 (s, 9H). ¹⁹F NMR (282 MHz, DMSO-d₆) δ −59.59. | calcd. for C₁₉H₂₁F₃N₄O₃ S: 442.1 m/z, found: 443.10 [M + H]⁺. |
| 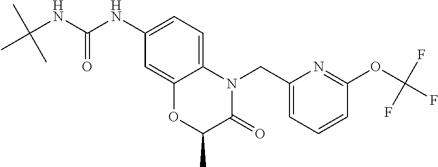 [SMILES: C[C@H]1Oc2cc(NC(=O)NC(C)(C)C)ccc2N(Cc2cccc(OC(F)(F)F)n2)C1=O] | 166 | (R)-1- (tert-butyl)- 3-(2- methyl-3- oxo-4-((6- (trifluoro- methoxy) pyridin-2- yl)methyl)- 3,4- dihydro-2H- benzo [b][1,4] oxazin-7- yl)urea | ¹H NMR (300 MHz, DMSO-d₆) δ 8.22 (s, 1H), 7.96-8.01 (m, 1H), 7.29-7.31 (m, 1H), 7.13-7.26 (m, 2H), 6.82-6.84 (m, 1H), 6.77-6.82 (m, 1H), 5.94 (s, 1H), 5.14 (s, 2H), 4.75-4.77 (m, 1H), 1.43-1.46 (d, J = 6 Hz, 3H), 1.27 (s, 9H) | calcd. for C₂₁H₂₃F₃N₄O₄: 452.17 m/z, found: 451.00 [M − H]⁻ |
| 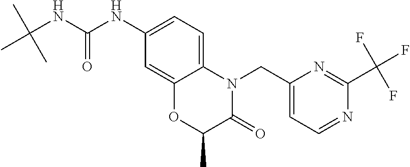 [SMILES: C[C@H]1Oc2cc(NC(=O)NC(C)(C)C)ccc2N(Cc2ccnc(C(F)(F)F)n2)C1=O] | 167 | (R)-1- (tert-butyl)- 3-(2- methyl-3- oxo-4-((2- (trifluoro- methyl) pyrimidin-4- yl)methyl)- 3,4- dihydro- 2H- benzo [b][1,4] oxazin-7- yl)urea | ¹H NMR (400 MHz, DMSO-d₆) δ 9.01 (d, J = 5.2 Hz, 1H), 8.25 (s, 1H), 7.68 (d, J = 5.2 Hz, 1H), 7.27 (d, J = 2.2 Hz, 1H), 6.67- 6.85 (m, 2H), 5.95 (s, 1H), 5.21-5.36 (m, 2H), 4.75-4.90 (m, 1H), 1.47 (d, J = 6.8 Hz, 3H), 1.27 (s, 9H). ¹⁹F NMR (376 MHz, DMSO) δ (ppm): −69.1848. | calcd. for C₂₀H₂₂F₃N₅O₃: 437.17 m/z, found 438.15 [M + H]⁺ |
| 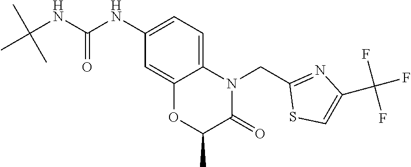 [SMILES: C[C@H]1Oc2cc(NC(=O)NC(C)(C)C)ccc2N(Cc2nc(C(F)(F)F)cs2)C1=O] | 168 | (R)-1- (tert-butyl)- 3-(2- methyl-3- oxo-4-((4- (trifluoro- methyl) thiazol-2- yl)methyl)- 3,4- dihydro-2H- benzo [b][1,4] oxazin-7- yl)urea | ¹H NMR (300 MHz, DMSO-d₆) δ 8.46 (s, 1H), 8.27 (s, 1H), 7.25- 7.26 (d, J = 3 Hz, 1H), 7.06-7.09 (m, 1H), 6.79-6.86 (m, 1H), 5.98 (s, 1H), 5.38- 5.51 (m, 2H), 4.78- 4.80 (m, 1H), 1.43- 1.45 (d, J = 6 Hz, 3H), 1.23-1.26 (m, 9H) | calcd. for C₁₉H₂₁F₃N₄O₃ S: 442.13 m/z, found 443.00 [M + H]⁺ |
| 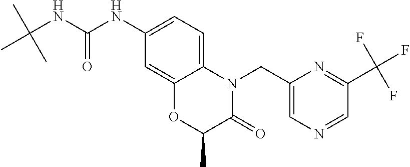 [SMILES: C[C@H]1Oc2cc(NC(=O)NC(C)(C)C)ccc2N(Cc2cncc(C(F)(F)F)n2)C1=O] | 169 | (R)-1- (tert-butyl)- 3-(2- methyl-3- oxo-4-((6- (trifluoro- methyl) pyrazin-2- yl)methyl)- 3,4- dihydro-2H- benzo [b][1,4] oxazin-7- yl)urea | ¹H NMR (300 MHz, DMSO-d₆) δ 9.11 (s, 1H), 8.98 (s, 1H), 8.23 (s, 1H), 7.22-7.23 (m, 1H), 6.88-6.90 (m, 1H), 6.76-6.79 (m, 1H), 5.95 (s, 1H), 5.34 (s, 2H), 4.78-4.83 (m, 1H), 1.43-1.45 (d, J = 6 Hz, 3H), 1.27 (s, 9H), 1.19 (s, 2H) | calcd. for C₂₀H₂₂F₃N₅O₃: 437.17 m/z, found: 438.00 [M + H]⁺ |
| 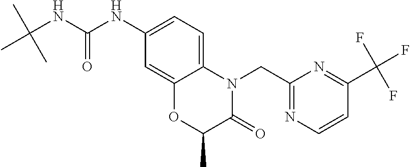 [SMILES: C[C@H]1Oc2cc(NC(=O)NC(C)(C)C)ccc2N(Cc2nccc(C(F)(F)F)n2)C1=O] | 170 | (R)-1- (tert-butyl)- 3-(2- methyl-3- oxo-4-((4- (trifluoro- methyl) pyrimidin-2- yl)methyl)- 3,4- dihydro-2H- benzo [b][1,4] oxazin-7- yl)urea | ¹H NMR (300 MHz, DMSO-d₆) δ 9.13-9.15 (d, J = 6 Hz, 1H), 8.22 (s, 1H), 7.93-7.95 (d, J = 6 Hz, 1H), 7.24 (s, 1H), 6.71-6.86 (m, 2H), 5.94 (s, 1H), 5.28-5.41 (m, J = 4.1 Hz, 2H), 4.72-4.79 (m, 1H), 1.42-1.44 (d, J = 6 Hz, 3H), 1.27 (s, 9H). ¹⁹F NMR (282 MHz, DMSO) δ −68.72 | C₂₀H₂₂F₃N₅O₃: 437.17 m/z, found: 438.00 [M + H]⁺ |
| 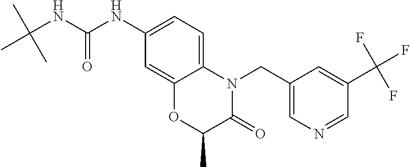 [SMILES: C[C@H]1Oc2cc(NC(=O)NC(C)(C)C)ccc2N(Cc2cncc(C(F)(F)F)c2)C1=O] | 171 | (R)-1- (tert-butyl)- 3-(2- methyl-3- oxo-4-((5- (trifluoro- methyl) pyridin-3- yl)methyl)- 3,4- dihydro-2H- benzo [b][1,4] oxazin-7- yl)urea | ¹H NMR (300 MHz, DMSO-d₆) δ 8.85-8.92 (m, 1H), 8.72-8.79 (m, 1H), 8.22 (s, 1H), 8.05 (s, 1H), 7.22 (s, 1H), 6.93-6.96 (m, 1H), 6.78-6.82 (m, 1H), 5.93 (s, 1H), 5.29-5.32 (m, 1H), 5.15-5.26 (m, 1H), 4.83-4.98 (m, 1H), 1.46-1.49 (d, J = 9 Hz, 3H), 1.26 (s, 9H). ¹⁹F NMR (282 MHz, DMSO) δ −60.93. | C₂₁H₂₃F₃N₄O₃: 436.17 m/z, found: 437.05 [M + H]⁺ |
| 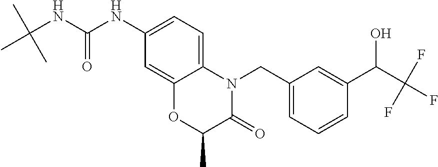 [SMILES: C[C@H]1Oc2cc(NC(=O)NC(C)(C)C)ccc2N(Cc2cccc(C(O)C(F)(F)F)c2)C1=O] | 172 | 1-(tert- butyl)-3- ((2R)-2- methyl-3- oxo-4- (3-(2,2,2- trifluoro-1- hydroxy- ethyl) benzyl)-3,4- dihydro- 2H- benzo [b][1,4] oxazin-7- yl)urea | ¹H NMR (300 MHz, DMSO-d₆) δ 8.20 (s, 1H), 7.39-7.35 (m, 3H), 7.21-7.20 (m, 2H), 6.83-6.81 (m, 2H), 6.79-6.77 (m, 1H), 5.93 (s, 1H), 5.15- 5.11 (m, 3H), 4.81 (q, J = 6.6 Hz, 1H), 1.47 (d, J = 6.6 Hz, 3H), 1.26 (s, 9H). ¹⁹F NMR (282 MHz, DMSO-d₆) δ −76.69. | calcd. for C₂₃H₂₆F₃N₃O₄: 465.2 m/z, found: 466.2 [M + H]⁺ |
| 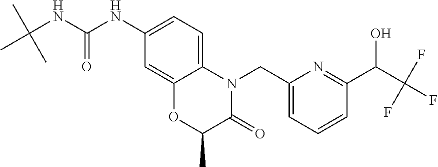 [SMILES: C[C@H]1Oc2cc(NC(=O)NC(C)(C)C)ccc2N(Cc2cccc(C(O)C(F)(F)F)n2)C1=O] | 173 | 1-(tert- butyl)-3- ((2R)-2- methyl-3- oxo-4-((6- (2,2,2- trifluoro-1- hydroxy- ethyl) pyridin-2-yl) methyl)- 3,4- dihydro-2H- benzo [b][1,4] oxazin-7- yl)urea | ¹H NMR (400 MHz, DMSO-d₆) δ 8.21 (s, 1H), 7.75-7.98 (m, 1H), 7.40-7.60 (m, 1H), 7.15-7.30 (m, 2H), 7.01 (d, J = 6.0 Hz, 1H), 6.88-6.78 (m, 1H), 6.65-6.78 (m, 1H), 5.93 (s, 1H), 4.96-5.30 (m, 3H), 4.70-4.90 (m, 1H), 4.86-4.76 (m, 1H), 1.47 (d, J = 6.7, 2.3 Hz, 3H), 1.26 (s, 9H). ¹⁹F NMR (376 MHz, DMSO) δ (ppm): 75.7620, −75.7743. | calcd. for C₂₂H₂₅F₃N₄O₄: 466.18 m/z, found 467.20 [M + H]⁺ |
| 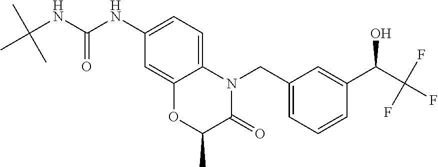 [SMILES: C[C@H]1Oc2cc(NC(=O)NC(C)(C)C)ccc2N(Cc2cccc([C@@H](O)C(F)(F)F)c2)C1=O] | 174 | 1-(tert- butyl)-3- ((R)-2- methyl-3- oxo-4- (3-((R)- 2,2,2- trifluoro-1- hydroxyethyl) benzyl)-3,4- dihydro- 2H- benzo [b][1,4] oxazin-7- yl)urea | ¹H NMR (300 MHz, DMSO-d₆) δ 8.21 (s, 1H), 7.39-7.35 (m, 3H), 7.23-7.21 (m, 2H), 6.86-6.74 (m, 3H), 5.94 (s, 1H), 5.12- 5.11 (m, 3H), 4.81 (q, J = 6.6 Hz, 1H), 1.47 (d, J = 6.6 Hz, 3H), 1.26 (s, 9H). ¹⁹F NMR (282 MHz, DMSO-d₆) δ −76.69. | calcd. for C₂₃H₂₆F₃N₃O₄: 465.3 m/z, found: 488.15 [M + Na]⁺ |
| 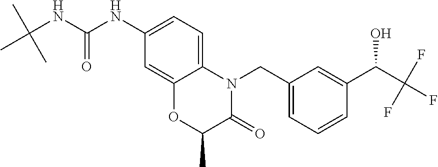 [SMILES: C[C@H]1Oc2cc(NC(=O)NC(C)(C)C)ccc2N(Cc2cccc([C@H](O)C(F)(F)F)c2)C1=O] | 175 | 1-(tert- butyl)-3- ((R)-2- methyl-3- oxo-4- (3-((S)- 2,2,2- trifluoro-1- hydroxyethyl) benzyl)-3,4- dihydro- 2H- benzo [b][1,4] oxazin-7- yl)urea | ¹H NMR (300 MHz, DMSO-d₆) δ 8.20 (s, 1H), 7.39-7.35 (m, 3H), 7.22-7.21 (m, 2H), 6.83-6.77 (m, 3H), 5.93 (s, 1H), 5.12- 5.11 (m, 3H), 4.81 (q, J = 6.6 Hz, 1H), 1.47 (d, J = 6.6 Hz, 3H), 1.26 (s, 9H). ¹⁹F NMR (282 MHz, DMSO-d₆) δ −76.69. | calcd. for C₂₃H₂₆F₃N₃O₄: 465.3 m/z, found: 488.15 [M + Na^{]+} |
| 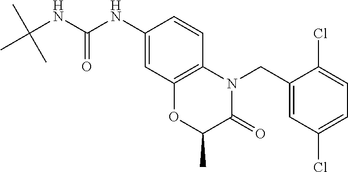 [SMILES: C[C@H]1Oc2cc(NC(=O)NC(C)(C)C)ccc2N(Cc2cc(Cl)ccc2Cl)C1=O] | 176 | (R)-1- (tert-butyl)- 3-(4-(2,5- dichloro- benzyl)-2- methyl-3- oxo-3,4- dihydro-2H- benzo [b][1,4] oxazin-7- yl)urea | ¹H NMR (300 MHz, DMSO-d₆) δ 8.27 (s, 1H), 7.53-7.62 (m, 1H), 7.36-7.46 (m, 1H), 7.29 (d, J = 3.0 Hz, 1H), 6.99 (d, J = 3.0 Hz, 1H), 6.76- 6.84 (m, 1H), 6.65- 6.74 (m, 1H), 5.96 (s, 1H), 5.14-5.23 (m, 2H), 4.86-5.05 (m, 1H), 1.49 (d, J = 9.0 Hz, 3H), 1.27 (s, 9H). | calcd. For C₂₁H₂₃Cl₂N₃O₃: 435.1 m/z, found: 436.10 [M + H]⁺. |
| 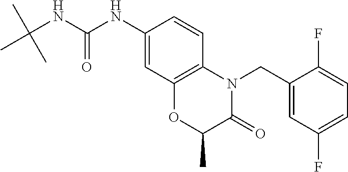 [SMILES: C[C@H]1Oc2cc(NC(=O)NC(C)(C)C)ccc2N(Cc2cc(F)ccc2F)C1=O] | 177 | (R)-1- (tert-butyl)- 3-(4-(2,5- difluoro- benzyl)-2- methyl-3- oxo-3,4- dihydro-2H- benzo [b][1,4] oxazin-7- yl)urea | ¹H NMR (300 MHz, DMSO-d₆) δ 8.24 (s, 1H), 7.11-7.38 (m, 3H), 6.88-6.97 (m, 1H), 6.75-6.88 (m, 2H), 5.95 (s, 1H), 5.11- 5.23 (m, 1H), 4.98- 5.10 (m, 1H), 4.79- 4.92 (m, 1H), 1.48 (d, J = 6.7 Hz, 3H), 1.27 (s, 9H). ¹⁹F NMR (282 MHz, DMSO) δ −118.31- 123.38. | calcd. for C₂₁H₂₃F₂N₃O₃: 403.17 m/z, found: 404.15 [M + H]+. |
| 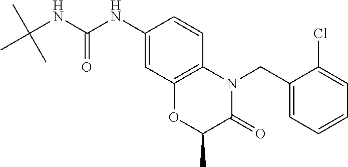 [SMILES: C[C@H]1Oc2cc(NC(=O)NC(C)(C)C)ccc2N(Cc2ccccc2Cl)C1=O] | 178 | (R)-1- (tert-butyl)- 3-(4-(2- chloro- benzyl)-2- methyl-3- oxo-3,4- dihydro-2H- benzo [b][1,4] oxazin-7- yl)urea | ¹H NMR (300 MHz, DMSO-d₆) δ 8.23 (s, 1H), 7.53-7.50 (m, 1H), 7.32-7.27 (m, 3H), 6.99-6.96 (m, 1H), 6.77-6.73 (m, 1H), 6.66 (d, J = 9.0 Hz, 1H), 5.94 (s, 1H), 5.17-5.00 (m, 2H), 4.86 (q, J = 6.9 Hz, 1H), 1.49 (d, J = 6.9 Hz, 3H), 1.26 (s, 9H). | calcd. for C₂₁H₂₄ClN₃O₃: 401.2 m/z, found: 402.10 [M + H]⁺ |

### Example 179: 3-tert-butyl-1-{3-methyl-2-oxo-1-[(1S)-1-phenylethyl]quinoxalin-6-yl}urea

### Synthetic Scheme

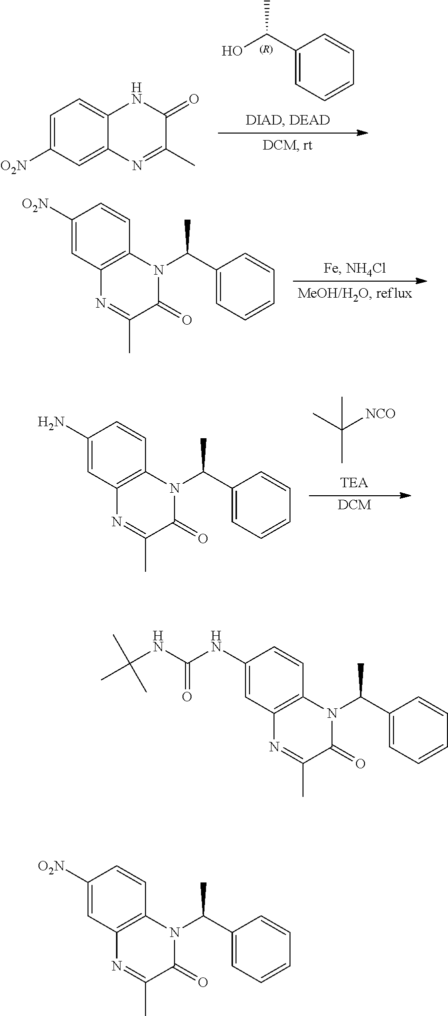

### (S)-3-methyl-6-nitro-1-(1-phenylethyl)quinoxalin-2(1H)-one

To a solution of 3-methyl-6-nitro-1H-quinoxalin-2-one (600 mg, 2.924 mmol, 1 equiv) and 1-phenyl-ethanol (357.26 mg, 2.924 mmol, 1 equiv) in DCM (10 mL) was added PPh₃ (1150.55 mg, 4.386 mmol, 1.5 equiv). After the reaction was stirred at rt for 30 min under N₂, DEAD (509.30 mg, 2.924 mmol, 1 equiv) was added at 0° C. The reaction was stirred at rt under N₂ for 3 h. Quenched with water (20 mL) and extracted with DCM (3×20 mL). The combined extracts were washed with water, brine, dried over Na₂SO₄, filtered and concentrated. The residue was purified by column chromatography on silica gel with EA/PE (0-100%) to give 3-methyl-6-nitro-1-[(1S)-1-phenylethyl]quinoxalin-2-one (217 mg, 23.99%) as a white solid. MS (ESI): mass calcd. for C₁₇H₁₅N₃O₃: 309.11 m/z, found 310.05 [M+H]+

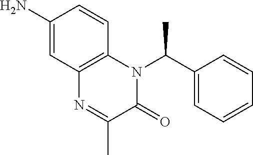 [SMILES: Cc1nc2cc(N)ccc2n([C@@H](C)c2ccccc2)c1=O]

### 6-amino-3-methyl-1-[(1S)-1-phenylethyl]quinoxalin-2-one

To a solution of 3-methyl-6-nitro-1-[(1S)-1-phenylethyl]quinoxalin-2-one (200 mg, 0.647 mmol, 1 equiv) in methanol (8 mL) and water (0.8 mL) was added iron (361.08 mg, 6.470 mmol, 10 equiv) and NH₄Cl (345.85 mg, 6.470 mmol, 10 equiv). The reaction was stirred at 60° C. for 3 h. After cooling down to rt, the reaction was quenched with water (30 mL) and extracted with EA (3×30 mL). The combined extracts were washed with water, brine, dried over Na₂SO₄, filtered and concentrated. The residue was purified by column chromatography on silica gel with EA/PE (0-100%) to give 6-amino-3-methyl-1-[(1S)-1-phenylethyl]quinoxalin-2-one (95 mg, 52.60%) as a white solid. MS (ESI): mass calcd. for C₁₇H₁₇N₃O: 279.14 m/z, found 280.10 [M+H]⁺

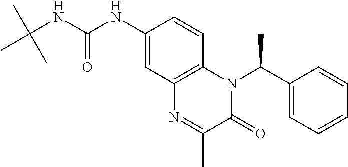 [SMILES: Cc1nc2cc(NC(=O)NC(C)(C)C)ccc2n([C@@H](C)c2ccccc2)c1=O]

### 3-tert-butyl-1-{3-methyl-2-oxo-1-[(1S)-1-phenylethyl]quinoxalin-6-yl}urea

To a solution of 6-amino-3-methyl-1-[(1S)-1-phenylethyl]quinoxalin-2-one (85 mg, 0.304 mmol, 1 equiv) and 2-isocyanato-2-methylpropane (60.33 mg, 0.608 mmol, 2 equiv) in DCM (2 mL) was added TEA (92.37 mg, 0.912 mmol, 3 equiv). The reaction was stirred at rt for 6h. Quenched with water (20 mL) and extracted with DCM (3×20 mL). The combined extracts were washed with water, brine, dried over Na₂SO₄, filtered and concentrated. The residue was purified by preparative HPLC using a XBridge Prep OBD C18 150 mm×30 mm×5 μm column (eluent: 17% to 42% (v/v) CH₃CN and H₂O with 10 mmol/L NH₄HCO₃) to afford the title compound 3-tert-butyl-1-{3-methyl-2-oxo-1-[(1S)-1-phenylethyl]quinoxalin-6-yl}urea (20.1 mg, 17.37%) as a white solid. LC/MS (ESI): mass calcd. for C₂₂H₂₆N₄O₂:378.21 m/z, found:379.05 [M+H]⁺. ¹H NMR (300 MHz, DMSO-d₆) δ 8.33 (s, 1H), 7.79 (s, 1H), 7.16-7.40 (m, 6H), 7.00 (s, 1H), 6.64 (s, 1H), 6.00 (s, 1H), 2.47-2.51 (m, 3H), 1.86-1.88 (d, J=6 Hz, 3H), 1.27 (s, 9H).

The following examples in Table 8 were made according to the procedure for example 179.

TABLE 8

| Structure | Ex. | IUPAC Name | 1H NMR | LCMS |
|---|---|---|---|---|
| 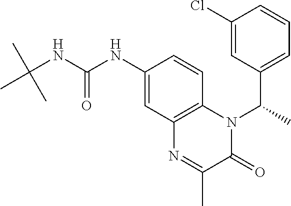 [SMILES: Cc1nc2cc(NC(=O)NC(C)(C)C)ccc2n([C@@H](C)c2cccc(Cl)c2)c1=O] | 180 | (S)-1-(tert- butyl)-3-(1-(1- (3- chlorophenyl) ethyl)-3-methyl-2- oxo-1,2- dihydroquinoxalin- 6-yl)urea | ¹H NMR (400 MHz, DMSO-d₆) δ 8.34 (s, 1H), 7.81 (s, 1H), 7.30-7.41 (m, 3H), 7.20-7.34 (m, 2H), 7.01 (s, 1H), 6.59 (s, 1H), 6.01 (s, 1H), 2.46-2.51 (m, 3H), 1.85-1.87 (d, J = 8 Hz, 3H), 1.29 (s, 9H) | calcd. for C₂₂H₂₅ClN₄O₂: 412.17 m/z, found: 413.00 [M + H]⁺ |
| 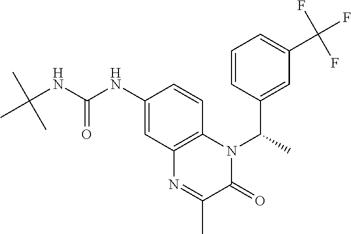 [SMILES: Cc1nc2cc(NC(=O)NC(C)(C)C)ccc2n([C@@H](C)c2cccc(C(F)(F)F)c2)c1=O] | 181 | (S)-1-(tert- butyl)-3-(3- methyl-2-oxo-1- (1-(3- (trifluoromethyl) phenyl)ethyl)- 1,2- dihydroquinoxalin- 6-yl)urea | ¹H NMR (400 MHz, DMSO-d₆) δ 8.36 (s, 1H), 7.81 (s, 1H), 7.49-7.68 (m, 4H), 7.29 (s, 1H), 7.08 (s, 1H), 6.62 (s, 1H), 6.02 (s, 1H), 2.45 (s, 3H), 1.90-1.92 (d, J = 8 Hz, 3H), 1.28 (s, 9H). ¹⁹F NMR (376 MHz, DMSO) δ −60.85. | calcd. for C₂₃H₂₅F₃N₄O₂: 446.19 m/z, found: 447.05 [M + H]⁺. |
| 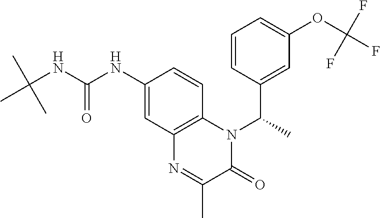 [SMILES: Cc1nc2cc(NC(=O)NC(C)(C)C)ccc2n([C@@H](C)c2cccc(OC(F)(F)F)c2)c1=O] | 182 | (S)-1-(tert- butyl)-3-(3- methyl-2-oxo-1- (1-(3- (trifluoromethoxy) phenyl)ethyl)- 1,2- dihydroquinoxalin- 6-yl)urea | ¹H NMR (300 MHz, DMSO-d₆) δ 8.35 (s, 1H), 7.80 (d, J = 2.4 Hz, 1H), 7.45 (d, J = 7.8 Hz, 1H), 7.28- 7.26 (m, 4H), 6.55 (s, 1H), 6.01 (s, 1H), 2.45 (s, 3H), 1.88 (d, J = 7.2 Hz, 3H), 1.28 (s, 9H). | calcd. for C₂₃H₂₅F₃N₄O₃: 462.2 m/z, found: 463.15 [M + H]⁺ |

### Example 183: 1-(tert-butyl)-3-((R)-4-((S)-1-(3-chlorophenyl)ethyl)-2-methyl-3-oxo-3,4-dihydro-2H-benzo[b][1,4]oxazin-7-yl)urea

### Synthetic Scheme

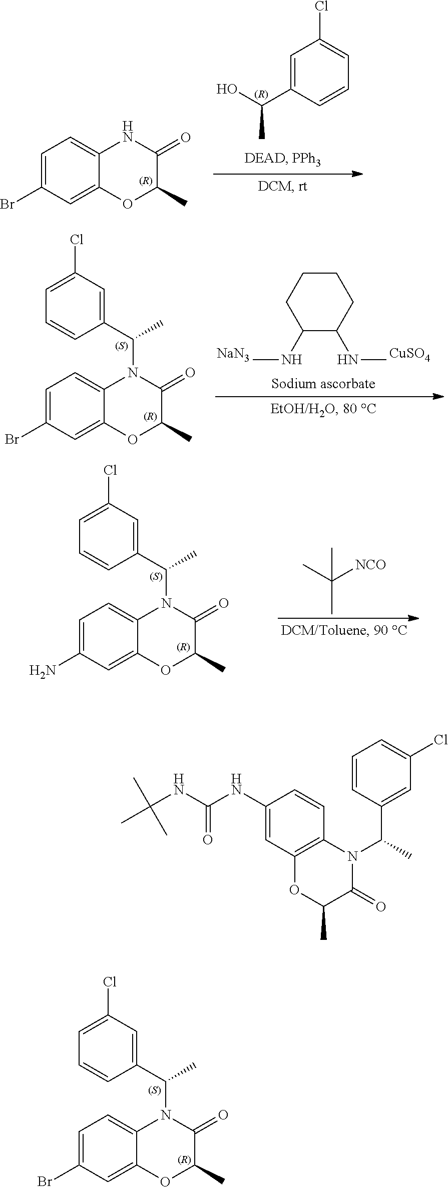

### (R)-7-bromo-4-((S)-1-(3-chlorophenyl)ethyl)-2-methyl-2H-benzo[b][1,4]oxazin-3(4H)-one

To a solution of (2R)-7-bromo-2-methyl-2,4-dihydro-1,4-benzoxazin-3-one (270 mg, 1.115 mmol, 1 equiv) in DCM (10 mL) was added 1-[(1R)-1-bromoethyl]-3-chlorobenzene (9.07 mg, 0.041 mmol, 1 equiv) and PPh₃ (438.83 mg, 1.672 mmol, 1.5 equiv). The reaction was stirred at rt under N₂ for 0.5 hour. To the above mixture was added DEAD (291.37 mg, 1.672 mmol, 1.5 equiv) in portions at 0° C. The resulting mixture was stirred for additional 1 hour at rt. Quenched with water (20 mL) and extracted with DCM (3×20 mL). The combined extracts were washed with water, brine, dried over Na₂SO₄, filtered and concentrated. The residue was purified by column chromatography on silica gel with EA/PE (0-100%) to give (2R)-7-bromo-4-[(1S)-1-(3-chlorophenyl)ethyl]-2-methyl-2H-1,4-benzoxazin-3-one (194 mg, 45.69%) as a white oil. MS (ESI): mass calcd. for C₁₇H₁₅BrClNO₂: 379.00m/z, found 380.95, 382.95 [M+H, M+H+2]⁺.

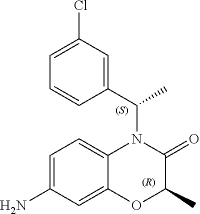

### (R)-7-amino-4-((S)-1-(3-chlorophenyl)ethyl)-2-methyl-2H-benzo[b][1,4]oxazin-3(4H)-one

To a solution of (2R)-7-bromo-4-[(1S)-1-(3-chlorophenyl)ethyl]-2-methyl-2H-1,4-benzoxazin-3-one (180 mg, 0.473 mmol, 1 equiv) in EtOH/H₂O (4:1, 10 mL) was added (1R,2R)—N1,N2-dimethylcyclohexane-1,2-diamine (40.36 mg, 0.284 mmol, 0.6 equiv), CuSO₄ (75.47 mg, 0.473 mmol, 1 equiv), azidosodium (92.22 mg, 1.419 mmol, 3 equiv) and sodium ascorbate (188.30 mg, 0.946 mmol, 2 equiv). The reaction was stirred at 80° C. under N₂ for 1 day. After cooling down to rt, the reaction was quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-7-amino-4-[(1S)-1-(3-chlorophenyl)ethyl]-2-methyl-2H-1,4-benzoxazin-3-one (110 mg, 73.43%) as a yellow oil. MS (ESI): mass calcd. for C₁₇H₁₇ClN₂O₂: 316.10m/z, found 317.15 [M+H]⁺.

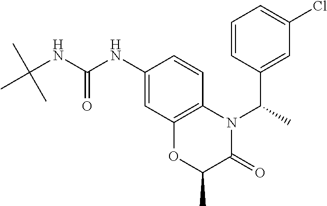 [SMILES: C[C@H]1Oc2cc(NC(=O)NC(C)(C)C)ccc2N([C@@H](C)c2cccc(Cl)c2)C1=O]

### 1-(tert-butyl)-3-((R)-4-((S)-1-(3-chlorophenyl)ethyl)-2-methyl-3-oxo-3,4-dihydro-2H-benzo[b][1,4]oxazin-7-yl)urea

To a solution of (2R)-7-amino-4-[(1S)-1-(3-chlorophenyl)ethyl]-2-methyl-2H-1,4-benzoxazin-3-one (100 mg, 0.316 mmol, 1 equiv) in DCM/Toluene (2:1, 12 mL) was added 2-isocyanato-2-methylpropane (312.93 mg, 3.160 mmol, 10 equiv). The reaction was stirred at 90° C. under N₂ for 3 h. After cooling down to rt, the reaction was quenched with water (10 mL) and extracted with DCM (3×10 mL). The combined extracts were washed with water, brine, dried over Na₂SO₄, filtered and concentrated. The residue was purified by preparative HPLC using a YMC-Actus Triart C18 ExRS, 30*150 mm, 5 μm column (eluent: 53% to 66% (v/v) CH₃CN and H₂O with 10 mmol/L NH₄HCO₃) to afford the title compound 3-tert-butyl-1-[(2R)-4-[(1S)-1-(3-chlorophenyl)ethyl]-2-methyl-3-oxo-2H-1,4-benzoxazin-7-yl]urea (27.5 mg, 20.95%) as a white solid. MS (ESI): mass calcd. for C₂₂H₂₆ClN₃O₃: 415.17 m/z, found:416.15 [M+H]⁺. ¹H NMR (400 MHz, DMSO-d₆) δ 8.07-8.37 (m, 1H), 7.30-7.60 (m, 3H), 7.00-7.30 (m, 2H), 6.52-6.74 (m, 2H), 5.83-6.10 (m, 2H), 4.55-4.88 (m, 1H), 1.66-1.85 (m, 3H), 1.40-1.53 (m, 3H), 1.22-1.30 (m, 9H).

The following examples in Table 9 were made according to the procedure for example 183.

TABLE 9

| Structure | Ex. | IUPAC Name | 1H NMR | LCMS |
|---|---|---|---|---|
| 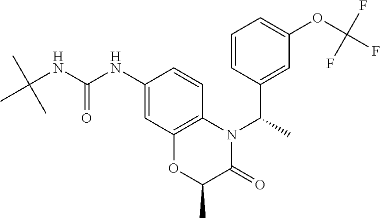 [SMILES: C[C@H]1Oc2cc(NC(=O)NC(C)(C)C)ccc2N([C@@H](C)c2cccc(OC(F)(F)F)c2)C1=O] | 184 | 1-(tert-butyl)-3- ((R)-2-methyl-3- oxo-4-((S)-1-(3- (trifluoromethoxy) phenyl)ethyl)-3,4- dihydro-2H- benzo[b][1,4] oxazin-7-yl)urea | ¹H NMR (300 MHz, DMSO-d₆) δ 8.20 (s, 1H), 7.50 (t, J = 8.0 Hz, 1H), 7.31-7.20 (m, 4H), 6.66-6.64 (m, 2H), 6.04 (q, J = 7.2 Hz, 1H), 5.94 (s, 1H), 4.74 (q, J = 6.9 Hz, 1H), 1.76 (d, J = 7.2 Hz, 3H), 1.44 (d, J = 6.0 Hz, 3H), 1.26 (s, 9H). | calcd. for C₂₃H₂₆F₃N₃O₄: 465.2 m/z, found: 466.15 [M + H]⁺. |
| 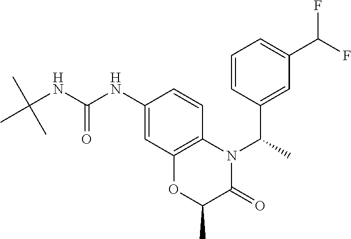 [SMILES: C[C@H]1Oc2cc(NC(=O)NC(C)(C)C)ccc2N([C@@H](C)c2cccc(C(F)F)c2)C1=O] | 185 | 1-(tert-butyl)-3- ((R)-4-((S)-1-(3- (difluoromethyl) phenyl)ethyl)-2- methyl-3-oxo-3,4- dihydro-2H- benzo[b][1,4] oxazin-7-yl)urea | ¹H NMR (400 MHz, DMSO-d₆) δ 8.08-8.30 (m, 1H), 7.33-7.65 (m, 4H), 7.23- 7.33 (m, 1H), 6.80- 7.20 (m, 1H), 6.52-6.76 (m, 2H), 6.00-6.20 (m, 1H), 5.72- 6.00 (m, 1H), 4.66- 4.83 (m, 1H), 1.68-1.88 (m, 3H), 1.40-1.60 (m, 3H), 1.18- 1.34 (m, 9H). ¹⁹F NMR (376 MHz, DMSO) δ (ppm): −109.432, −109.5495. | calcd. for C₂₃H₂₇F₂N₃O₃: 431.20 m/z, found 432.20 [M + H]⁺ |
| 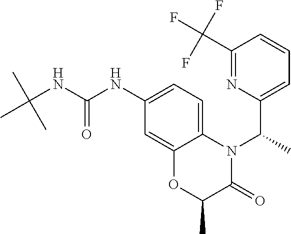 [SMILES: C[C@H]1Oc2cc(NC(=O)NC(C)(C)C)ccc2N([C@@H](C)c2cccc(C(F)(F)F)n2)C1=O] | 186 | 1-(tert-butyl)-3- ((R)-2-methyl-3- oxo-4-((S)-1-(6- (trifluoromethyl) pyridin-2-yl)ethyl)- 3,4-dihydro-2H- benzo[b][1,4] oxazin-7-yl)urea | ¹H NMR (400 MHz, DMSO-d₆) δ 8.14-8.39 (m, 1H), 8.00-8.14 (m, 1H), 7.68- 7.86 (m, 1H), 7.53- 7.67 (m, 1H), 7.03-7.30 (m, 1H), 6.53-6.80 (m, 2H), 6.00- 6.25 (m, 1H), 5.72- 6.00 (m, 1H), 4.38-4.83 (m, 1H), 1.70-1.87 (m, 3H), 1.36- 1.54 (m, 3H), 1.20- 36 (m, 9H). ¹⁹F NMR (376 MHz, DMSO) δ (ppm): −66.5649. | calcd. for C₂₄H₂₉N₃O₄: 450.19 m/z, found 451.20 [M + H]⁺ |
| 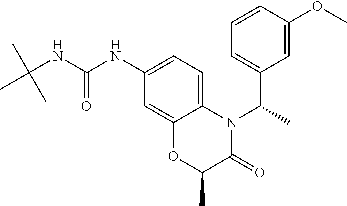 [SMILES: COc1cccc([C@H](C)N2C(=O)[C@@H](C)Oc3cc(NC(=O)NC(C)(C)C)ccc32)c1] | 187 | 1-(tert-butyl)-3- ((R)-4-((S)-1-(3- methoxyphenyl) ethyl)-2-methyl-3- oxo-3,4-dihydro- 2H- benzo[b][1,4] oxazin-7-yl)urea | ¹H NMR (300 MHz, DMSO-d₆) δ 8.18 (s, 1H), 7.21- 7.34 (m, 2H), 6.78- 6.89 (m, 3H), 6.54- 6.67 (m, 2H), 6.05- 6.07 (m, 1H), 5.94 (s, 1H), 4.74-4.76 (m, 1H), 3.72 (s, 3H), 1.71-1.73 (d, J = 6 Hz, 3H), 1.41- 1.43 (d, J = 6 Hz, 3H), 1.26 (s, 9H). | calcd. for C₂₃H₂₉N₃O₄: 411.22 m/z, found: 412.05 [M + H]⁺ |
| 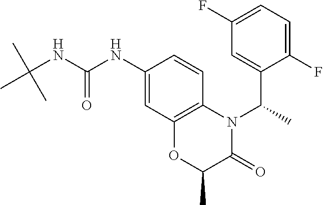 [SMILES: C[C@H]1Oc2cc(NC(=O)NC(C)(C)C)ccc2N([C@@H](C)c2cc(F)ccc2F)C1=O] | 188 | 1-(tert-butyl)-3- ((R)-4-((S)-1-(2,5- difluorophenyl) ethyl)-2-methyl-3- oxo-3,4-dihydro- 2H- benzo[b][1,4] oxazin-7-yl)urea | ¹H NMR (300 MHz, DMSO-d₆) δ 8.18 (s, 1H), 7.32- 7.50 (m, 1H), 7.03- 7.28 (m, 3H), 6.85- 7.02 (m, 1H), 6.66- 6.84 (m, 1H), 5.96- 6.09 (m, 1H), 5.93 (s, 1H), 4.59 (q, J = 6.6 Hz, 1H), 1.77 (d, J = 7.1 Hz, 3H), 1.41 (d, J = 6.7 Hz, 3H),1.26 (s, 9H). ¹⁹F NMR (282 MHz, DMSO) δ −118.57, −121.56. | calcd. for C₂₂H₂₅F₂N₃O₃: 417.19 m/z, found: 418.20 [M + H]⁺ |
| 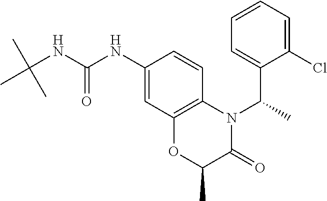 [SMILES: C[C@H]1Oc2cc(NC(=O)NC(C)(C)C)ccc2N([C@@H](C)c2ccccc2Cl)C1=O] | 189 | 1-(tert-butyl)-3- ((R)-4-((S)-1-(2- chlorophenyl)ethyl)- 2-methyl-3-oxo- 3,4-dihydro-2H- benzo[b][1,4] oxazin-7-yl)urea | ¹H NMR (300 MHz, DMSO-d₆) δ 8.14 (s, 1H), 7.77 (dd, J = 7.8, 1.6 Hz, 1H), 7.22-7.44 (m, 3H), 7.10 (d, J = 2.4 Hz, 1H), 6.88 (d, J = 8.8 Hz, 1H), 6.69 (dd, J = 8.8, 2.4 Hz, 1H), 5.80- 5.97 (m, 2H), 4.55 (q, J = 6.6 Hz, 1H), 1.82 (d, J = 7.1 Hz, 3H), 1.44 (d, J = 6.7 Hz, 3H), 1.25 (s, 9H). | calcd. For C₂₂H₂₆ClN₃O₃: 415.17 m/z, found: 416.20 [M + H]⁺. |
| 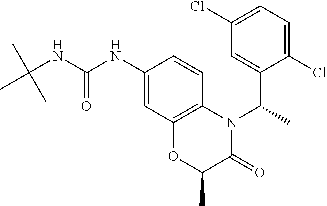 [SMILES: C[C@H]1Oc2cc(NC(=O)NC(C)(C)C)ccc2N([C@@H](C)c2cc(Cl)ccc2Cl)C1=O] | 190 | 1-(tert-butyl)-3- ((R)-4-((S)-1-(2,5- dichlorophenyl) ethyl)-2-methyl-3- oxo-3,4-dihydro- 2H- benzo[b][1,4] oxazin-7-yl)urea | ¹H NMR (300 MHz, DMSO-d₆) δ 8.20 (d, J = 13.3 Hz, 1H), 7.78 (d, J = 1.7 Hz, 1H), 7.37 (d, J = 1.5 Hz, 2H), 7.11 (d, J = 2.4 Hz, 1H), 6.98 (d, J = 8.9 Hz, 1H), 6.76 (dd, J = 8.8, 2.4 Hz, 1H), 5.94 (d, J = 6.9 Hz, 1H), 5.70- 5.85 (m, 1H), 4.55 (q, J = 6.7 Hz, 1H), 1.81 (d, J = 6.9 Hz, 3H), 1.41 (d, J = 6.7 Hz, 3H), 1.20- 1.28 (m, 9H). | calcd. for C₂₂H₂₅Cl₂N₃O₃: 449.13 m/z, found 450.15 [M + H]⁺ |

### Example 191: 1-(1-benzyl-2-oxo-1,2-dihydroquinoxalin-6-yl)-3-(1-methylcyclobutyl)urea

### Synthetic Scheme

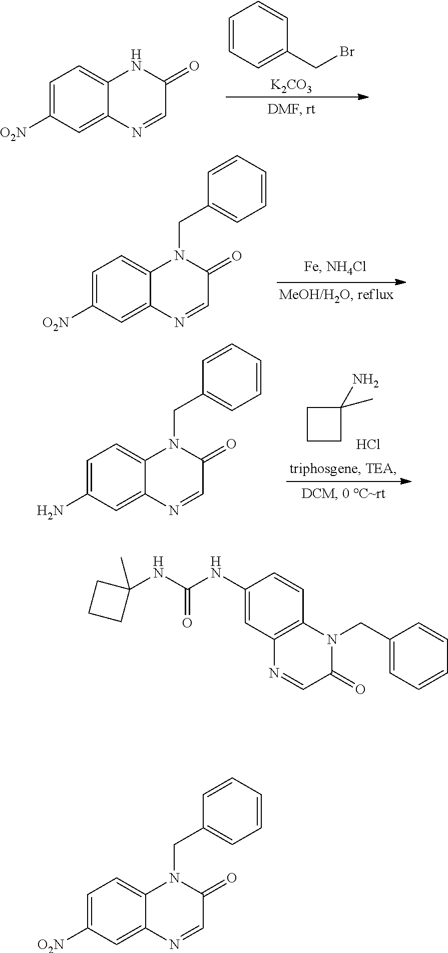

### benzyl-6-nitroquinoxalin-2(1H)-one

To a solution of 6-nitro-1H-quinoxalin-2-one (3 g, 15.695 mmol, 1 equiv) and benzyl bromide (5368.79 mg, 31.390 mmol, 2 equiv) in dimethylformamide (100 mL) was added Cs₂CO₃ (15388.46 mg, 47.085 mmol, 3 equiv). The reaction was stirred at rt for 3 h. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give 1-benzyl-6-nitroquinoxalin-2-one (3.1 g, 70.22%) as a yellow solid. MS (ESI): mass calcd. for C₁₅H₁₁N₃O₃: 281.08 m/z, found 282.10 [M+H]⁺

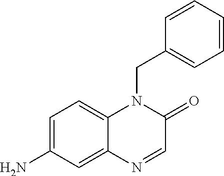 [SMILES: Nc1ccc2c(c1)ncc(=O)n2Cc1ccccc1]

### 6-amino-1-benzylquinoxalin-2(1H)-one

To a solution of 1-benzyl-6-nitroquinoxalin-2-one (3.1 g, 11.021 mmol, 1 equiv) in methanol (80 mL) and water (8 mL) was added iron (6.15 g, 110.210 mmol, 10 equiv) and NH₄Cl (5.90 g, 110.210 mmol, 10 equiv). The reaction was stirred at 60° C. for 6 h. After cooling down to rt, the reaction was quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give 6-amino-1-benzylquinoxalin-2-one (1 g, 36.11%) as a red solid. MS (ESI): mass calcd. for C₁₅H₁₃N₃O: 251.11 m/z, found 252.10 [M+H]⁺

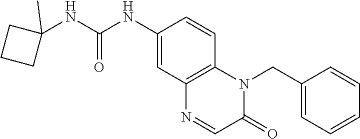 [SMILES: CC1(NC(=O)Nc2ccc3c(c2)ncc(=O)n3Cc2ccccc2)CCC1]

### 1-(1-benzyl-2-oxo-1,2-dihydroquinoxalin-6-yl)-3-(1-methylcyclobutyl)urea

To a solution of 6-amino-1-benzylquinoxalin-2-one (100 mg, 0.398 mmol, 1 equiv) in DCM (3 mL) was added triphosgene (47.23 mg, 0.159 mmol, 0.4 equiv). The reaction was stirred at 0° C. under N₂ for 1 h. The reaction was added TEA (201.35 mg, 1.990 mmol, 5 equiv) and stirred at rt under N₂ for 1 h. The reaction was added 1-methylcyclobutan-1-amine (67.77 mg, 0.796 mmol, 2 equiv) and stirred at rt under N₂ for 1 h. Quenched with water (10 mL) and extracted with DCM (3×10 mL). The combined extracts were washed with water, brine, dried over Na₂SO₄, filtered and concentrated. The residue was purified by preparative HPLC using a XBridge Prep OBD C18 Column 21.2 mm×250 mm, 5 m column (eluent: 17% to 42% (v/v) CH₃CN and H₂O with 10 mmol/L NH₄HCO₃) to afford the title compound 1-(1-benzyl-2-oxoquinoxalin-6-yl)-3-(1-methylcyclobutyl)urea (32.5 mg, 22.48%) as a white solid. LC/MS (ESI): mass calcd. for C₂₁H₂₂N₄O₂:362.17 m/z, found:363.00 [M+H]⁺. ¹H NMR (300 MHz, DMSO-d₆) δ 8.47 (s, 1H), 8.33 (s, 1H), 7.99 (s, 1H), 7.42-7.43 (1, 1H), 7.20-7.39 (m, 6H), 6.32 (s, 1H), 5.45 (s, 2H), 2.24-2.28 (m, 2H), 1.68-1.96 (m, 4H), 1.40 (s, 3H).

TABLE 10

| Structure | Ex. | IUPAC Name | 1H NMR | LCMS |
|---|---|---|---|---|
| 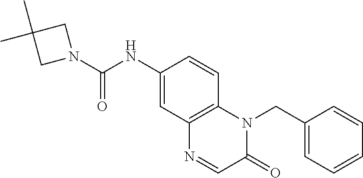 [SMILES: CC1(C)CN(C(=O)Nc2ccc3c(c2)ncc(=O)n3Cc2ccccc2)C1] | 192 | N-(1-benzyl-2-oxo- 1,2- dihydroquinoxalin- 6-yl)-3,3- dimethylazetidine- 1-carboxamide | ¹H NMR (300 MHZ, DMSO-d₆) δ 8.57 (s, 1H), 8.32 (s, 1H), 8.06 (s, 1H), 7.65-7.69 (m, 1H), 7.20-7.38 (m, 6H), 5.45 (s, 2H), 3.65 (s, 4H), 1.24 (s, 6H). | calcd. for C₂₁H₂₂N₄O₂: 362.17 m/z, found: 363.00 [M + H]⁺. |
| 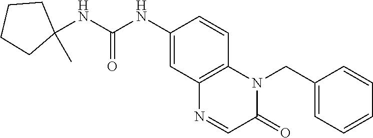 [SMILES: CC1(NC(=O)Nc2ccc3c(c2)ncc(=O)n3Cc2ccccc2)CCCC1] | 193 | 1-(1-benzyl-2-oxo- 1,2- dihydroquinoxalin- 6-yl)-3-(1- methylcyclopentyl) urea | ¹H NMR (300 MHZ, DMSO-d₆) δ 8.44 (s, 1H), 8.34 (s, 1H), 7.99 (s, 1H), 7.39-7.44 (m, 1H), 7.32-7.43 (m, 3H), 7.24-7.37 (m, 3H), 6.11 (s, 1H), 5.46 (s, 2H), 1.88-1.93 (m, 2H), 1.50-1.65 (m, | calcd. for C₂₂H₂₄N₄O₂: 376.19 m/z, found: 377.05 [M + H]⁺. |
| | | | 6H), 1.36 (s, 3H). | |
| 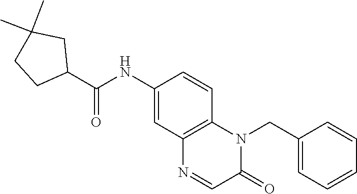 [SMILES: CC1(C)CCC(C(=O)Nc2ccc3c(c2)ncc(=O)n3Cc2ccccc2)C1] | 194 | N-(1-benzyl-2-oxo- 1,2- dihydroquinoxalin- 6-yl)-3,3- dimethylpyrrolidine- 1-carboxamide | ¹H NMR (300 MHZ, DMSO-d₆) δ 8.21-8.39 (m, 2H), 8.04-8.11 (m, 1H), 7.62-7.78 (m, 1H), 7.20-7.41 (m, 6H), 5.46 (s, 2H), 3.47 (t, J = 7.0 Hz, 2H), 3.16 (s, 2H), 1.62-1.73 (m, 2H), 1.07 (s, 6H). | calcd. for C₂₂H₂₄N₄O₂: 376.2 m/z, found: 377.20 [M + H]⁺ |
| 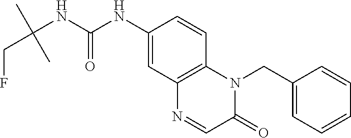 [SMILES: CC(C)(CF)NC(=O)Nc1ccc2c(c1)ncc(=O)n2Cc1ccccc1] | 195 | 1-(1-benzyl-2-oxo- 1,2- dihydroquinoxalin- 6-yl)-3-(1-fluoro-2- methylpropan-2- yl)urea | ¹H NMR (300 MHZ, DMSO-d₆) δ 8.58 (s, 1H), 8.32 (s, 1H), 7.97 (d, J = 2.4 Hz, 1H), 7.24 - 7.41 (m, 7H), 6.16 (s, 1H), 5.45 (s, 2H), 4.54 (s, 1H), 4.38 (s, 1H), 1.27 (d, J = 2.0 Hz, 6H). | calcd. for C₂₀H₂₁FN₄O₂, 368.16 m/z, found 369.10 [M + H]⁺ |
| 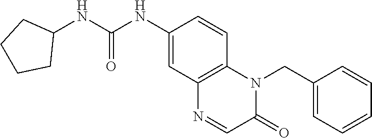 [SMILES: O=C(Nc1ccc2c(c1)ncc(=O)n2Cc1ccccc1)NC1CCCC1] | 196 | 1-(1-benzyl-2-oxo- 1,2- dihydroquinoxalin- 6-yl)-3- cyclopentylurea | ¹H NMR (300 MHZ, DMSO-d₆) δ 8.49 (s, 1H), 8.32 (s, 1H), 8.00 (d, J = 2.5 Hz, 1H), 7.42-7.52 (m, 1H), 7.29 - 7.39 (m, 2H), 7.20-7.33 (m, 4H), 6.24 (d, J = 7.2 Hz, 1H), 5.45 (s, 2H), | calcd. for C₂₁H₂₂N4O₂: 362.2 m/z, found: 363.10 [M + H]⁺ |
| | | | 3.86-4.01 (m, 1H), | |
| | | | 1.76 - 1.92 (m, 2H), | |
| | | | 1.48 - 1.69 (m, 4H), | |
| | | | 1.29-1.45 (m, 2H). | |
| 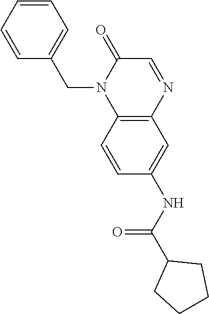 [SMILES: O=C(Nc1ccc2c(c1)ncc(=O)n2Cc1ccccc1)C1CCCC1] | 197 | N-(1-benzyl-2-oxo- 1,2 dihydroquinoxalin- 6- yl)cyclopentanecar- boxamide | ¹H NMR (300 MHZ, DMSO-d₆) δ 10.09 (s, 1H), 8.35 (s, 1H), 8.22 (d, J = 2.4 Hz, 1H), 7.70 (dd, J = 9.1, 2.5 Hz, 1H), 7.43 (d, J = 9.1 Hz, 1H), 7.20- 7.38 (m, 5H), 5.47 (s, 2H), 2.77 (p, J = 7.7 Hz, 1H), 1.80 - 1.94 (m, 2H), 1.55-1.80 (m, 6H). | calcd. for C₂₁H₂₁N₃O₂: 347.2 m/z, found: 348. [M + H]⁺ |
| 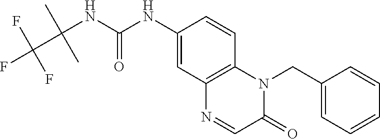 [SMILES: CC(C)(NC(=O)Nc1ccc2c(c1)ncc(=O)n2Cc1ccccc1)C(F)(F)F] | 198 | 1-(1-benzyl-2-oxo- 1,2- dihydroquinoxalin- 6-yl)-3-(1,1,1- trifluoro-2- methylpropan-2- yl)urea | ¹H NMR (400 MHZ, DMSO-d₆) δ 8.61 (s, 1H), 8.34 (s, 1H), 7.98 (d, J = 2.4 Hz, 1H), 7.23 - 7.45 (m, 7H), 6.57 (s, 1H), 5.46 (s, 2H), 1.52 (s, 6H). | calcd. for C₂₀H₁₉F₃N₄O₂, 404.15 m/z, found 405.05 [M + H].⁺ |
| 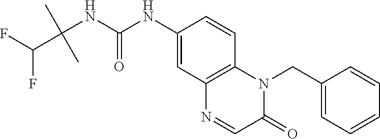 [SMILES: CC(C)(NC(=O)Nc1ccc2c(c1)ncc(=O)n2Cc1ccccc1)C(F)F] | 199 | 1-(1-benzyl-2-oxo- 1,2- dihydroquinoxalin- 6-yl)-3-(1,1- difluoro-2- methylpropan-2- yl)urea | ¹H NMR (300 MHZ, DMSO-d₆) δ 8.63 (s, 1H), 8.33 (s, 1H), 7.98 (s, 1H), 7.24-7.44 (m, 7H), 6.15-6.53 (m, 2H), 5.45 (s, 2H), 1.32 (s, 6H). 19F NMR (282 MHz, DMSO) δ - 131.24. | calcd. for C₂₀H₂₀F₂N₄O₂: O₂:386.16 m/z, found: 387.00 [M + H]⁺. |

The following examples in Table 10 were made according to the procedure for example 191.

### Example 200: (S)-1-(1-methylcyclobutyl)-3-(2-oxo-1-(1-(3-(trifluoromethoxy)phenyl)ethyl)-1,2-dihydroquinoxalin-6-yl)urea

### Synthetic Scheme

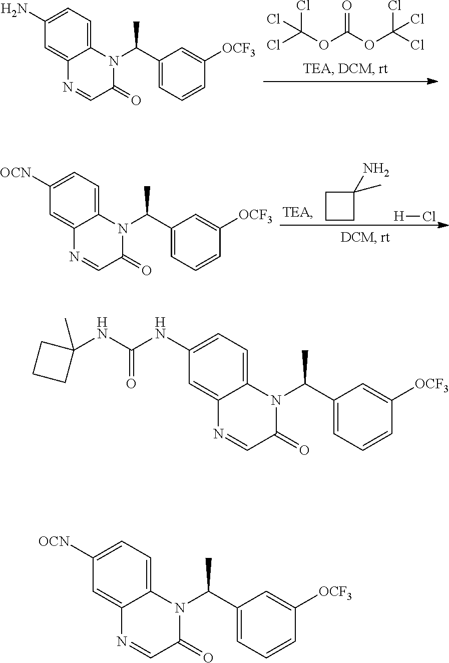

### (S)-6-isocyanato-1-(1-(3-(trifluoromethoxy)phenyl)ethyl)quinoxalin-2(1H)-one

To a stirred solution of (S)-6-amino-1-(1-(3-(trifluoromethoxy)phenyl)ethyl)quinoxalin-2(1H)-one (70 mg, 0.200 mmol, 1 equiv) in DCM (5 mL) was added TEA (81 mg, 0.802 mmol, 4 equiv) and triphosgene (24 mg, 0.080 mmol, 0.4 equiv) at 0° C. under a nitrogen atmosphere, the reaction was stirred 1 h at rt. The reaction progress was monitored by LCMS. The reaction mixture was quenched by water (5 mL) and extracted with DCM (3×10 mL). The combined organic extracts were concentrated under vacuum to yield a 70 mg crude (S)-6-isocyanato-1-(1-(3-(trifluoromethoxy)phenyl)ethyl)quinoxalin-2(1H)-one as a yellow solid.

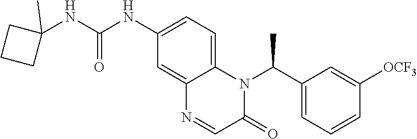 [SMILES: C[C@@H](c1cccc(OC(F)(F)F)c1)n1c(=O)cnc2cc(NC(=O)NC3(C)CCC3)ccc21]

### (S)-1-(1-methylcyclobutyl)-3-(2-oxo-1-(1-(3-(trifluoromethoxy)phenyl)ethyl)-1,2-dihydroquinoxalin-6-yl)urea

To a stirred solution of (S)-6-isocyanato-1-(1-(3-(trifluoromethoxy)phenyl)ethyl)quinoxalin-2(1H)-one (70 mg, 0.187 mmol, 1 equiv) in DCM (5 mL) was added TEA (57 mg, 0.560 mmol, 3 equiv) and 1-methylcyclobutanamine hydrochloride (27 mg, 0.224 mmol, 1.2 equiv) at 0° C. under a nitrogen atmosphere, the reaction was stirred 1 h at rt. The reaction progress was monitored by LCMS. The reaction mixture was quenched by water (10 mL) and extracted with DCM (3×10 mL). The combined organic extracts were washed with brine (20 mL), dried over anhydrous Na₂SO₄, and concentrated under vacuum to yield a crude product which was directly purified by flash chromatography (0-100% ethyl acetate/petroleum ether) to afford (S)-1-(1-methylcyclobutyl)-3-(2-oxo-1-(1-(3-(trifluoromethoxy)phenyl)ethyl)-1,2-dihydroquinoxalin-6-yl)urea (9.2 mg, 10.67%) as a yellow solid. MS (ESI): mass calcd. for C₂₃H₂₃F₃N₄O₃, 460.17 m/z, found 461.10 [M+H]⁺. ¹H NMR (300 MHz, DMSO-d₆) δ 8.44 (s, 1H), 8.24 (s, 1H), 7.95 (d, J=2.5 Hz, 1H), 7.47 (t, J=8.3 Hz, 1H), 7.29 (d, J=8.2 Hz, 4H), 7.11 (s, 1H), 6.57 (s, 1H), 6.31 (s, 1H), 2.28 (q, J=9.5 Hz, 2H), 1.89 (d, J=7.0 Hz, 4H), 1.76 (dd, J=9.2, 6.0 Hz, 3H), 1.39 (s, 3H).

The following examples in Table 11 were made according to the procedure for example 200.

TABLE 11

| Structure | Ex. | IUPAC Name | 1H NMR | LCMS |
|---|---|---|---|---|
| 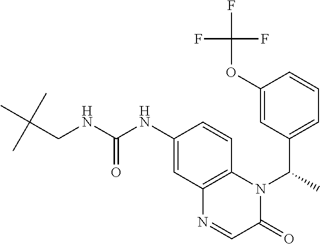 [SMILES: C[C@@H](c1cccc(OC(F)(F)F)c1)n1c(=O)cnc2cc(NC(=O)NCC(C)(C)C)ccc21] | 201 | (S)-1-neopentyl- 3-(2-oxo-1-(1- (3- (trifluoromethoxy) phenyl)ethyl)- 1,2- dihydroquinoxalin- 6-yl)urea | ¹H NMR (300 MHZ, DMSO-d₆) δ 8.61 (s, 1H), 8.25 (s, 1H), 7.99 (d, J = 2.5 Hz, 1H), 7.48 (dd, J = 10.6, 6.0 Hz, 1H), 7.36 (d, J = 8.9 Hz, 1H), 7.29 (d, J = 7.2 Hz, 3H), 7.11 (s, 1H), 6.58 (s, 1H), 6.24 (t, J = 6.1 Hz, 1H), 2.92 (d, J = 6.0 Hz, 2H), 1.90 (d, J = 7.0 Hz, 3H), 0.86 (s, 9H) | calcd. for C₂₃H₂₅F₃N₄O₃, 462.19 m/z, found 463.10 [M + H]⁺ |
| 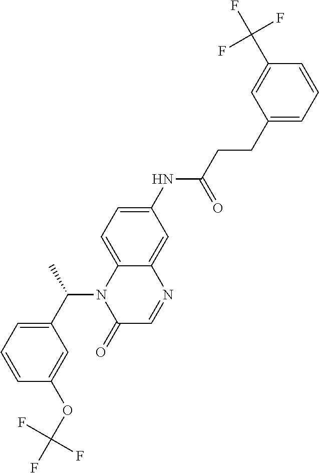 [SMILES: C[C@@H](c1cccc(OC(F)(F)F)c1)n1c(=O)cnc2cc(NC(=O)CCc3cccc(C(F)(F)F)c3)ccc21] | 202 | (S)-N-(2-oxo-1- (1-(3- (trifluoromethoxy) phenyl)ethyl)- 1,2- dihydroquinoxalin- 6-yl)-3-(3- (trifluoromethyl) phenyl) propanamide | ¹H NMR (300 MHZ, DMSO-d₆) δ 10.14 (s, 1H), 8.27 (s, 1H), 8.14 (d, J = 2.4 Hz, 1H), 7.42-7.66 (m, 6H), 7.00-7.40 (m, 4H), 6.57 (s, 1H), 3.02 (t, J = 7.5 Hz, 2H), 2.69 (t, J = 7.6 Hz, 2H), 1.90 (d, J = 7.0 Hz, 3H). ¹⁹F NMR (282 MHz, DMSO) δ −56.70, 61.00. | calcd. for C₂₇H₂₁F₆N₃O₃: 549.15, found: 571.95 [M + H]⁺ |
| 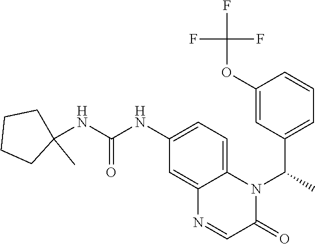 [SMILES: C[C@@H](c1cccc(OC(F)(F)F)c1)n1c(=O)cnc2cc(NC(=O)NC3(C)CCCC3)ccc21] | 203 | (S)-1-(1- methylcyclopent yl)-3-(2-oxo-1- (1-(3- (trifluoromethox y)phenyl)ethyl)- 1,2- dihydroquinoxal in-6-yl)urea | ¹H NMR (300 MHZ, DMSO-d₆) δ 8.40 (s, 1H), 8.25 (s, 1H), 7.95 (d, J = 3 Hz, 1H), 7.42-7.54 (m, 1H), 7.26-7.29 (m, 4H), 7.12 (s, 1H), 6.58 (s, 1H), 6.11 (s, 1H), 1.87 - 1.89 (m, 5H), 1.41-1.69 (m, 6H), 1.35 (s, 2H). ¹⁹F NMR (282 MHZ, DMSO-d₆) δ −56.67. | calcd. for C₂₄H₂₅F₃N₄O₃: 474.2 m/z, found: 475.15 [M + H]⁺ |
| 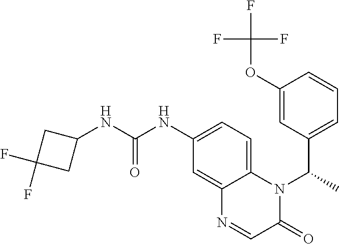 [SMILES: C[C@@H](c1cccc(OC(F)(F)F)c1)n1c(=O)cnc2cc(NC(=O)NC3CC(F)(F)C3)ccc21] | 204 | (S)-1-(3,3- difluorocyclobut yl)-3-(2-oxo-1- (1-(3- (trifluoromethoxy) phenyl)ethyl)- 1,2- dihydroquinoxalin- 6-yl)urea | ¹H NMR (300 MHZ, DMSO-d₆) δ 8.74 (s, 1H), 8.25 (s, 1H), 7.96-7.98 (m, 1H), 7.46-7.48 (m, 1H), 7.37-7.39 (m, 1H), 7.27- 7.29 (m, 3H), 7.12 (s, 1H), 6.75-6.77 (d, J = 6 Hz, 1H), 6.57 (s, 1H), 4.00- 4.03 (m, 1H), 2.82-2.93 (m, 2H), 2.72-2.73 (m, 2H), 1.87-1.89 (d, J = 6 Hz, 3H). ¹⁹F NMR (282 MHz, DMSO) δ −56.68, −82.40, −96.16. | calcd. for C₂₂H₁₉F₅N₄O₃: 482.14 m/z, found: 481 [M − H]⁻ |
| 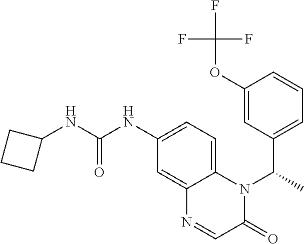 [SMILES: C[C@@H](c1cccc(OC(F)(F)F)c1)n1c(=O)cnc2cc(NC(=O)NC3CCC3)ccc21] | 205 | (S)-1- cyclobutyl-3-(2- oxo-1-(1-(3- (trifluoromethox y)phenyl)ethyl)- 1,2- dihydroquinoxal in-6-yl)urea | ¹H NMR (300 MHZ, DMSO-d₆) δ 8.53 (s, 1H), 8.24 (s, 1H), 7.96 (d, J = 2.5 Hz, 1H), 7.42-7.51 (m, 1H), 7.32-7.40 (m, 1H), 7.22-7.31 (m, 3H), 7.10 (s, 1H), 6.40-6.70 (m, 2H), 4.02-4.22 (m, 1H), 2.19 (q, J = 7.7 Hz, 2H), 1.79-1.98 (m, 5H), 1.50 - 1.70 (m, 2H). ¹⁹F NMR (282 MHz, DMSO- d₆) δ −56.67. | calcd. for C₂₂H₂₁F₃N₄O₃: 446.4 m/z, found: 447.10 [M + H]⁺ |
| 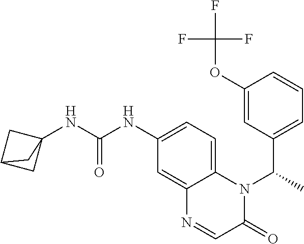 [SMILES: C[C@@H](c1cccc(OC(F)(F)F)c1)n1c(=O)cnc2cc(NC(=O)NC34CC(C3)C4)ccc21] | 206 | (S)-1- (bicycl[1.1.1] pentan-1-yl)-3-(2- oxo-1-(1-(3- (trifluoromethoxy) phenyl)ethyl)- 1,2- dihydroquinoxalin- 6-yl)urea | ¹H NMR (300 MHZ, DMSO-d₆) δ 8.47 (s, 1H), 8.25 (s, 1H), 7.96 (s, 1H), 7.44-7.49 (m, 1H), 7.25- 7.39 (m, 4H), 7.11 (s, 1H), 6.86 (s, 1H), 6.57 (s, 1H), 2.40 (s, 1H), 1.98 (s, 6H), 1.87-1.89 (d, J = 6 Hz, 3H). ¹⁹F NMR (282 MHZ, DMSO) δ −56.68. | calcd. for C₂₃H₂₁F₃N₄O₃: 458.1 6 m/z, found: 457.00 [M − H]⁻ |
| 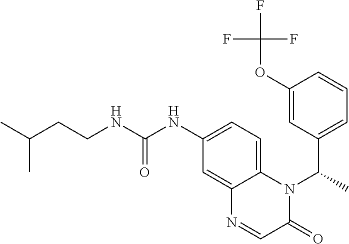 [SMILES: CC(C)CCNC(=O)Nc1ccc2c(c1)ncc(=O)n2[C@@H](C)c1cccc(OC(F)(F)F)c1] | 207 | (S)-1-isopentyl- 3-(2-oxo-1-(1- (3- (trifluoromethoxy) phenyl)ethyl)- 1,2- dihydroquinoxalin- 6-yl)urea | ¹H NMR (300 MHZ, DMSO-d₆) δ 8.59 (s, 1H), 8.24 (s, 1H), 7.97 (d, J = 2.5 Hz, 1H), 7.40-7.53 (m, 1H), 7.33-7.53 (m, 1H), 7.20-7.32 (m, 3H), 6.90-7.20 (m, 1H), 6.46- 6.69 (m, 1H), 6.0 - 6.21 (m, 1H), 3.02-3.18 (m, 2H), 1.89 (d, J = 7.0 Hz, 3H), 1.47-1.73 (m, 1H), 1.25-1.44 (m, 2H), 0.89 (d, J = 6.7 Hz, 6H). ¹⁹F NMR (282 MHZ, DMSO-d₆) δ −56.67. | calcd. for C₂₃H₂₅F₃N₄O₃: 462.2 m/z, found: 463.15 [M + H]⁺. |
| 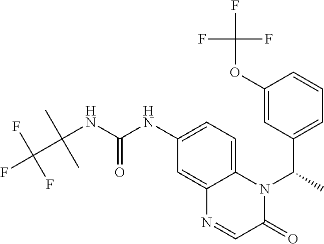 [SMILES: C[C@@H](c1cccc(OC(F)(F)F)c1)n1c(=O)cnc2cc(NC(=O)NC(C)(C)C(F)(F)F)ccc21] | 208 | (S)-1-(2-oxo-1- (1-(3- (trifluoromethox y)phenyl)ethyl)- 1,2- dihydroquinoxal in-6-yl)-3- (1,1,1-trifluoro- 2-methylpropan- 2-yl)urea | 1H NMR (300 MHZ, DMSO-d6) δ 8.59 (s, 1H), 8.26 (s, 1H), 7.95 (s, 1H), 7.42-7.54 (m, 1H), 7.21- 7.30 (m, 4H), 7.01 (s, 1H), 6.58 (s, 2H), 1.87-1.90 (d, J = 9 Hz, 3H), 1.52 (s, 6H). 19F NMR (282 MHZ, DMSO) δ −56.69. | calcd. for C₂₂H₂₀F₆N₄O₃: 403:502.1 4 m/z, found: 501.00 [M − H]⁻ |
| 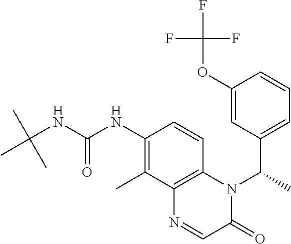 [SMILES: Cc1c(NC(=O)NC(C)(C)C)ccc2c1ncc(=O)n2[C@@H](C)c1cccc(OC(F)(F)F)c1] | 209 | (S)-1 -(tert- butyl)-3-(5- methyl-2-oxo-1- (1-(3- (trifluoromethoxy) phenyl)ethyl)- 1,2- dihydroquinoxal in-6-yl)urea | ¹H NMR (300 MHz, DMSO-d₆) δ 8.26 (s, 1H), 7.88-7.90 (d, J = 6 Hz, 1H), 7.67 (s, 1H), 7.40- 7.52 (m, 1H), 7.25-7.27 (m, 3H), 7.00 (s, 1H), 6.60 (s, 1H), 6.42 (s, 1H), 2.48 (s, 3H), 1.87-1.90 (m, 3H), 1.28 (s, 9H). ¹⁹F NMR (282 MHz, DMSO) δ −56.65. | calcd. for C₂₃H₂₅F₃N₄O₃: 462.1 9 m/z, found: 461.00 [M − H]⁻ |
| 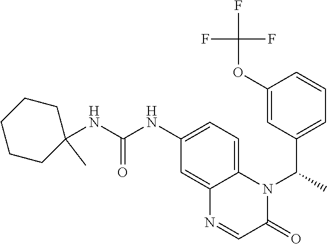 [SMILES: C[C@@H](c1cccc(OC(F)(F)F)c1)n1c(=O)cnc2cc(NC(=O)NC3(C)CCCCC3)ccc21] | 210 | (S)-1-(1- methylcyclohexyl)- 3-(2-oxo-1- (1-(3- (trifluoromethoxy) phenyl)ethyl)- 1,2- dihydroquinoxal in-6-yl)urea | ¹H NMR (300 MHz, DMSO-d6) δ 8.51 (s, 1H), 8.24 (s, 1H), 7.94 (d, J = 2.7 Hz, 1H), 7.54-7.42 (m, 1H), 7.29-7.27 (m, 4H), 7.11 (s, 1H), 6.58 (s, 1H), 5.86 (s, 1H), 1.90 - 1.88 (m, 5H), 1.45 - 1.43 (m, 5H), 1.30 - 1.28 (m, 6H). ¹⁹F NMR (282 MHZ, DMSO-d⁶) δ[56.67. | calcd. for C₂₅H₂₇F₃N₄O₃: 488.2 m/z, found: 489.2 [M + H]⁺. |
| 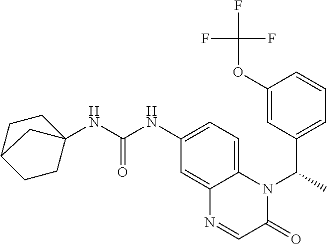 [SMILES: C[C@@H](c1cccc(OC(F)(F)F)c1)n1c(=O)cnc2cc(NC(=O)NC34CCC(CC3)C4)ccc21] | 211 | (S)-1- (bicyclo[2.2.1] heptan-1-yl)-3-(2- oxo-1-(1-(3- (trifluoromethoxy) phenyl)ethyl)- 1,2- di- hydroquinoxalin- 6-yl)urea | ¹H NMR (300 MHZ, DMSO-d6) δ 8.48 (s, 1H), 8.24 (s, 1H), 7.95 (d, J = 2.4 Hz, 1H), 7.47 (t, J = 8.2 Hz, 1H), 7.30 (d, J = 11.1 Hz, 4H), 7.08 (s, 1H), 6.56 (s, 1H), 6.45 (s, 1H), 2.09 (s, 1H), 1.89 (d, J = 7.2 Hz, 3H), 1.76-1.62 (m, 4H), 1.57-1.52 (m, 4H), 1.36-1.30 (m, 2H). 19F NMR (282 MHZ, DMSO-d₆) δ −56.67. | calcd. for C₂₅H₂₅F₃N₄O₃: 486.2 m/z, found: 487.15 [M + H]⁺. |
| 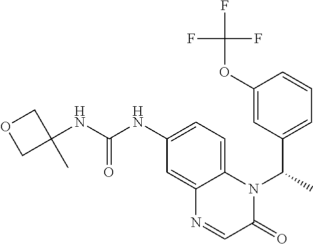 [SMILES: C[C@@H](c1cccc(OC(F)(F)F)c1)n1c(=O)cnc2cc(NC(=O)NC3(C)COC3)ccc21] | 212 | (S)-1-(3- methyloxetan-3- yl)-3-(2-oxo-1- (1-(3- (trifluoromethoxy) phenyl)ethyl)- 1,2- di- hydroquinoxalin)- 6-yl)urea | ¹H NMR (300 MHZ, DMSO-d₆) δ 8.71 (s, 1H), 8.25 (s, 1H), 7.96 (d, J = 2.5 Hz, 1H), 7.4 -7.53 (m, 1H), 7.34-7.43 (m, 1H), 7.20 - 7.32 (m, 3H), 7.11 (s, 1H), 6.71 (s, 1H), 6.57 (s, 1H), 4.52-4.70 (m, 2H), 4.20-4.34(m, 2H), 1.89 (d, J = 7.1 Hz, 3H), 1.55 (s, 3H). ¹⁹F NMR (282 MHz, DMSO) δ(ppm): −56.678. | calcd. for C₂₂H₂₁F₃N₄O₄: 462.15 m/z, found 463.15 [M + H]⁺ |
| 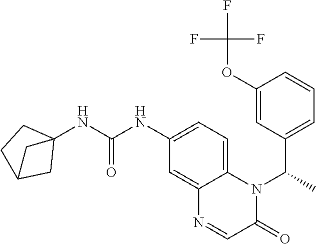 [SMILES: C[C@@H](c1cccc(OC(F)(F)F)c1)n1c(=O)cnc2cc(NC(=O)NC34CCC(C3)C4)ccc21] | 213 | (S)-1- (bicyclo[2.1.1] hexan-1-yl)-3-(2- oxo-1-(1-(3- (trifluoromethoxy) phenyl)ethyl)- 1,2- dihydroquinoxalin- 6-yl)urea | ¹H NMR (300 MHZ, DMSO-d₆) δ 8.48 (s, 1H), 8.25 (s, 1H), 7.96 (s, 1H), 7.44-7.50 (m, 1H), 7.27- 7.34 (m, 4H), 7.07 (s, 1H), 6.73 (s, 1H), 6.57 (s, 1H), 2.27 (s, 1H), 1.87-1.89 (m, 5H), 1.58-1.69 (m, 4H), 1.21 - 1.34 (m, 2H). ¹⁹F NMR (282 MHz, DMSO) δ −56.68. | calcd. for C₂₄H₂₃F₃N₄O₃: 472.1 7 m/z, found:473.05 [M + H]⁺. |
| 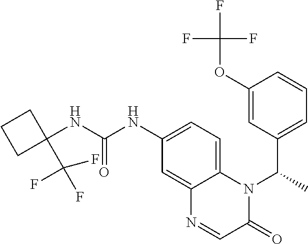 [SMILES: C[C@@H](c1cccc(OC(F)(F)F)c1)n1c(=O)cnc2cc(NC(=O)NC3(C(F)(F)F)CCC3)ccc21] | 214 | (S)-1-(2-oxo-1- (1-(3- (trifluoromethox y)phenyl)ethyl)- 1,2- dihydroquinoxalin- 6-yl)-3-(1- (trifluoromethyl) cyclobutyl)urea | ¹H NMR (400 MHZ, DMSO-d₆) δ 8.60 (s, 1H), 8.26 (s, 1H), 7.86- 8.07(m,1H), 7.43-7.61 (m, 1H), 7.28-7.38 (m, 4H), 6.96-7.21(m, 1H), 6.82- 6.96 (m, 1H), 6.34-6.65 (m, 1H), 2.38-2.49 (m, 4H), 1.96-2.09 (m, 1H), 1.84-1.96 (m, 4H). ¹⁹F NMR (376 MHz, DMSO) δ −56.68, −79.81. | calcd. for C₂₃H₂₀F₆N₄O₃: 514.1 m/z, found: 515.15 [M + H]⁺. |
| 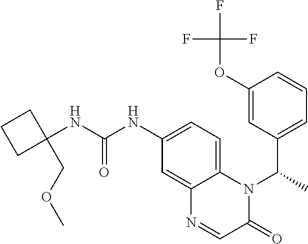 [SMILES: COCC1(NC(=O)Nc2ccc3c(c2)ncc(=O)n3[C@@H](C)c2cccc(OC(F)(F)F)c2)CCC1] | 215 | (S)-1-(1- (methoxymethyl) cyclobutyl)-3- (2-oxo-1-(1-(3- (tri- fluoromethoxy) phenyl)ethyl)- 1,2- dihydroquinoxalin- 6-yl)urea | ¹H NMR (400 MHZ, DMSO-d₆) δ 8.67(s, 1H), 8.22(s, 1H), 7.99(s, 1H), 7.38-7.58(m, 1H), 7.23-7.37(m, 4H),7.02 (s, 1H), 6.60 (s, 1H), 6.22 (s, 1H), 3.50 (s, 2H),3.29- 3.38 (m, 3H), 2.22-2.34 (m, 2H), 1.95-2.05 (m, 2H), 1.88-1.93 (m, 3H), 1.78-1.87 (m, 1H), 1.69-1.77 (m, 1H). ¹⁹F NMR (376 MHz, DMSO) δ −56.68. | calcd. C₂₄H₂₅F₃N₄O₄: 490.1 m/z, found: 491.15 [M + H]⁺. |
| 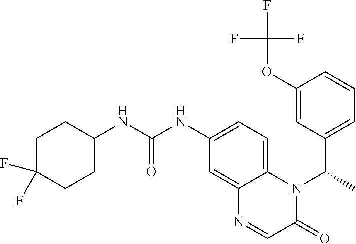 [SMILES: C[C@@H](c1cccc(OC(F)(F)F)c1)n1c(=O)cnc2cc(NC(=O)NC3CCC(F)(F)CC3)ccc21] | 216 | (S)-1-(4,4- di- fluorocyclohexyl)- 3-(2-oxo-1- (1-(3- (trifluoromethoxy) phenyl)ethyl)- 1,2- dihydroquinoxalin-) 6-yl)urea | ¹H NMR (400 MHZ, DMSO-d₆) δ 8.50 (s, 1H), 8.25 (s, 1H), 7.91-8.05 (m, 1H), 7.44-7.58 (m, 1H), 7.26-7.41 (m, 4H), 6.98- 7.22 (m, 1H), 6.43-6.69 (m, 1H), 6.30-6.38 (m, 1H), 3.57-3.79 (m, 1H), 1.84-2.05 (m, 9H), 1.42- 1.61 (m, 2H), 1.21-1.30 (m, 1H). ¹⁹F NMR (376 MHZ, DMSO) δ −56.82 | C₂₄H₂₃F₅N₄O₃: 510.4 m/z, found: 511.20 [M + H]⁺. |
| 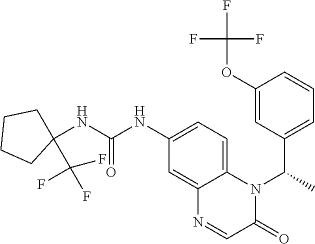 [SMILES: C[C@@H](c1cccc(OC(F)(F)F)c1)n1c(=O)cnc2cc(NC(=O)NC3(C(F)(F)F)CCCC3)ccc21] | 217 | (S)-1-(2-oxo-1- (1-(3- (trifluoromethoxy) phenyl)ethyl)- 1,2- dihydroquinoxalin)- 6-yl)-3-(1- (trifluoromethyl) cyclopentyl)urea | ¹H NMR (400 MHZ, DMSO-d₆) δ 8.51 (s, 1H), 8.23 (s, 1H), 7.98 (s, 1H), 7.41-7.59 (m, 1H),7.28- 7.39 (m, 4H), 7.08 (s, 1H), 6.52 (s, 2H), 2.20-2.31(m, 2H), 1.84-2.04(m, 5H), 1.58-1.78(m, 4H). ¹⁹F NMR (376 MHz, DMSO) δ −56.67, −75.81. | calcd. for C₂₄H₂₂F₆N₄O₃: 528.1 m/z, found: 529.20 [M + H]⁺. |
| 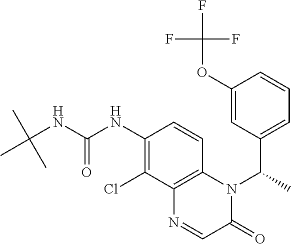 [SMILES: C[C@@H](c1cccc(OC(F)(F)F)c1)n1c(=O)cnc2c(Cl)c(NC(=O)NC(C)(C)C)ccc21] | 218 | (S)-1 -(tert- butyl)-3-(5- chloro-2-oxo-1- (1-(3- (trifluoromethoxy) phenyl)ethyl)- 1,2- dihydroquinoxalin- 6-yl)urea | ¹H NMR (300 MHZ, DMSO-d₆) δ 8.34 (s, 1H), 8.22-8.23 (m, 1H), 8.10 (s, 1H), 7.44-7.49 (m, 1H), 7.27-7.30 (m, 4H), 6.95 (s, 1H), 6.59 (s, 1H), 1.87- 1.90 (d, J = 9 Hz, 3H), 1.29 (s, 9H). ¹⁹F NMR (282 MHz, DMSO) δ −56.66. | calcd. for C₂₂H₂₂ClF₃N₄O₃: 482.13 m/z, found: 483.00 [M + H]⁺. |
| 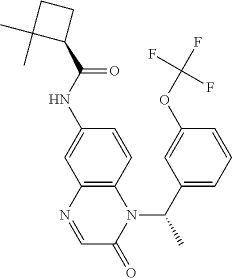 [SMILES: C[C@@H](c1cccc(OC(F)(F)F)c1)n1c(=O)cnc2cc(NC(=O)[C@@H]3CCC3(C)C)ccc21] | 219 | (S)-2,2- dimethyl-N-(2- oxo-1-((S)-1-(3- (trifluoromethoxy) phenyl)ethyl)- 1,2- dihydroquinoxalin- 6-yl) cyclobutane- 1-carboxamide | ¹H NMR (300 MHZ, DMSO-d6) δ 9.78 (s, 1H), 8.27 (s, 1H), 8.20 (s, 1H), 7.58-7.60 (m, 1H), 7.44- 7.49 (m, 1H), 7.27-7.30 (m, 3H), 7.18 (s, 1H), 6.58 (s, 1H), 2.85-2.90 (m, 1H), 2.19-2.28 (m, 1H), 1.78- 1.94 (m, 4H), 1.57-1.78 (m, 2H), 1.24 (s, 3H), 0.97 (s, 3H). ¹⁹F NMR (282 MHz, DMSO) δ −56.68. | calcd. for C₂₄H₂₄F₃N₃O₃: 459.18 m/z, found: 460.05 [M + H]⁺ |
| 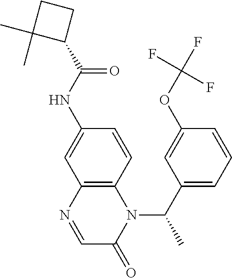 [SMILES: C[C@@H](c1cccc(OC(F)(F)F)c1)n1c(=O)cnc2cc(NC(=O)[C@H]3CCC3(C)C)ccc21] | 220 | (S)-2,2- dimethyl-N-(2- oxo-1-((R)-1-(3- (trifluoromethoxy) phenyl)ethyl)- 1,2- dihydroquinoxalin- 6-yl) cyclobutane- 1-carboxamide | ¹H NMR (300 MHZ, DMSO-d6) δ 9.78 (s, 1H), 8.27 (s, 1H), 8.19 (s, 1H), 7.58-7.61 (m, 1H), 7.44- 7.49 (m, 1H), 7.29 (s, 3H), 7.19 (s, 1H), 6.57 (s, 1H), 2.84-2.90 (m, 1H), 2.22- 2.28 (m, 1H), 1.88-1.90 (d, J = 6.0 Hz, 4H), 1.59-1.79 (m, 2H), 1.25 (s, 3H), 0.97 (s, 3H). ¹⁹F NMR (282 MHz, DMSO) δ −56.68. | calcd. for C₂₄H₂₄F₃N₃O₃: 459.18 m/z, found: 460.05 [M + H]⁺. |
| 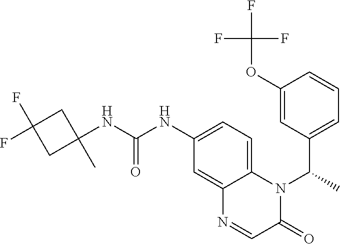 [SMILES: C[C@@H](c1cccc(OC(F)(F)F)c1)n1c(=O)cnc2cc(NC(=O)NC3(C)CC(F)(F)C3)ccc21] | 221 | (S)-1-(3,3- difluoro-1- methylcyclobuty 1)-3-(2-oxo-1-(1- (3- (trifluoromethoxy) phenyl)ethyl)- 1,2- dihydroquinoxalin- 6-yl)urea | ¹H NMR (400 MHZ, DMSO-d6) δ 8.62 (s, 1H), 8.25 (s, 1H), 7.91-8.01 (m, 1H), 7.42-7.53 (m, 1H), 7.31-7.42 (m, 1H), 7.23-7.31 (m, 3H), 6.92- 7.19 (m, 1H), 6.65-6.79(m, 1H), 6.46-6.65 (m, 1H), 2.83-3.01 (m, 2H), 2.55- 2.70 (m, 2H), 1.82-1.94(m, 3H), 1.42-1.51 (m, 3H).19F NMR (376 MHz, DMSO) δ −56.68, −85.00, −89.66. | calcd. For C₂₃H₂₁F₅N₄O₃: 496.1 m/z, found:497.15 [M + H]⁺. |
| 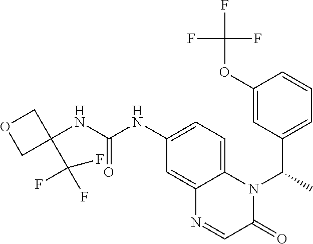 [SMILES: C[C@@H](c1cccc(OC(F)(F)F)c1)n1c(=O)cnc2cc(NC(=O)NC3(C(F)(F)F)COC3)ccc21] | 222 | (S)-1-(2-oxo-1- (1-(3- (trifluoromethoxy) phenyl)ethyl)- 1,2- dihydroquinoxalin)- 6-yl)-3-(3- (trifluoromethyl) oxetan-3-yl)urea | ¹H NMR (400 MHZ, DMSO-d₆) δ 8.93 (s, 1H), 8.26 (s, 1H), 7.90-8.07 (m, 1H), 7.38-7.63 (m, 3H), 7.24-7.38 (m, 3H), 6.95- 7.25 (m, 1H), 6.34-6.72 (m, 1H), 4.74-4.93 (m, 2H), 4.63-4.74 (m, 2H), 1.85-1.95(m, 3H). ¹⁹F NMR (376 MHz, DMSO) δ −56.68, −79.66. | calcd. for C₂₂H₁₈F₆N₄O₄: 516.1 m/z, found: 517.15 [M + H]⁺. |
| 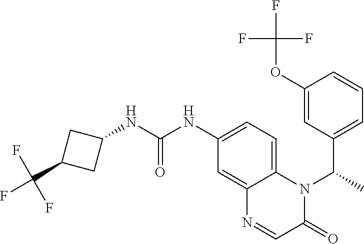 [SMILES: C[C@@H](c1cccc(OC(F)(F)F)c1)n1c(=O)cnc2cc(NC(=O)N[C@H]3C[C@H](C(F)(F)F)C3)ccc21] | 223 | 1-(2-oxo-1-((S)- 1-(3- (trifluoromethoxy) phenyl)ethyl)- 1,2- dihydroquinoxal in-6-yl)-3- ((1r,3R)-3- (trifluoromethyl) cyclobutyl)urea | ¹H NMR (300 MHZ, DMSO-d₆) δ 8.66 (s, 1H), 8.24 (s, 1H), 7.96 (s, 1H), 7.47-7.49 (m, 1H), 7.36- 7.44 (m, 1H), 7.25-7.33 (m, 3H), 7.11 (s, 1H), 6.65-6.69 (m, 1H), 6.57 (s, 1H), 4.28 (m, 1H), 2.98- 3.08 (m, 1H), 2.28-2.47 (m, 4H), 1.87-1.89 (d, J = 6.0 Hz, 3H). 19F NMR (282 MHz, DMSO) δ −56.69, −71.44. | calcd. for C₂₃H₂₀F₆N₄O₃: 514.14 m/z, found: 515.05 [M + H]⁺. |
| 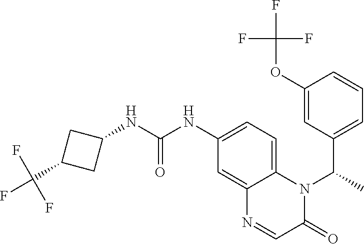 [SMILES: C[C@@H](c1cccc(OC(F)(F)F)c1)n1c(=O)cnc2cc(NC(=O)N[C@H]3C[C@@H](C(F)(F)F)C3)ccc21] | 224 | 1-(2-oxo-1-((S)- 1-(3- (trifluoromethoxy) phenyl)ethyl)- 1,2- dihydroquinoxalin- 6-yl)-3- ((1s,3S)-3- (trifluoromethyl) cyclobutyl)urea | ¹H NMR (300 MHZ, DMSO-d6) δ 8.55 (s, 1H), 8.24 (s, 1H), 7.96 (s, 1H), 7.46-7.49 (m, 1H), 7.35- 7.38 (m, 1H), 7.27-7.29 (m, 3H), 7.12 (s, 1H), 6.69-6.72 (m, 1H), 6.57 (s, 1H), 4.12-4.20 (m, 1H), 2.83-2.92 (m, 1H), 2.36- 2.48 (m, 2H), 1.98-2.02 (m, 2H), 1.87-1.92 (m, 3H). ¹⁹F NMR (282 MHZ, DMSO) δ −56.69, −71.04. | calcd. for C₂₃H₂₀F₆N₄O₃: 514.14 m/z, found:515.05 [M + H]⁺. |
| 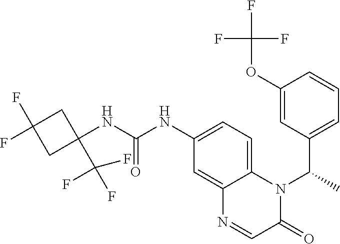 [SMILES: C[C@@H](c1cccc(OC(F)(F)F)c1)n1c(=O)cnc2cc(NC(=O)NC3(C(F)(F)F)CC(F)(F)C3)ccc21] | 225 | (S)-1-(3,3- difluoro-1- (trifluoromethyl) cyclobutyl)-3- (2-oxo-1-(1-(3- (trifluoromethoxy) phenyl)ethyl)- 1,2- dihydroquinoxalin- 6-yl)urea | ¹H NMR (400 MHZ, DMSO-d6) δ 8.77 (s, 1H), 8.26 (s, 1H), 7.96 (s, 1H), 7.43-7.52 (m, 1H), 7.37- 7.41 (m, 2H), 7.28-7.30 (m, 3H), 7.12 (s, 1H), 6.58 (s, 1H), 3.15-3.18 (t, J = 12 Hz, 4H), 1.88-1.90 (d, J = 8 Hz, 3H). ¹⁹F NMR (376 MHz, DMSO) δ −56.69, −78.33, −86.27. | calcd. for C₂₃H₁₈F₈N₄O₃: 550.13 m/z, found: 551.00 [M + H]⁺. |

### Example 226: 1-[(2R)-2-methyl-3-oxo-4-{[6-(trifluoromethyl)pyridin-2-yl]methyl}-2H-1,4-benzoxazin-7-yl]-3-(1-methylcyclobutyl)urea

### Synthetic Scheme

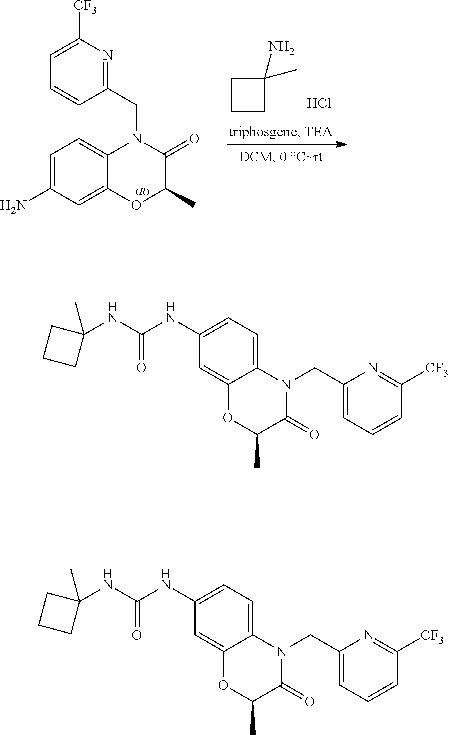

### 1-[(2R)-2-methyl-3-oxo-4-{[6-(trifluoromethyl)pyridin-2-yl]methyl}-2H-1,4-benzoxazin-7-yl]-3-(1-methylcyclobutyl)urea

To a solution of (2R)-7-amino-2-methyl-4-{[6-(trifluoromethyl)pyridin-2-yl]methyl}-2H-1,4-benzoxazin-3-one (100 mg, 0.296 mmol, 1 equiv) in DCM (5 mL) was added triphosgene (35.19 mg, 0.118 mmol, 0.4 equiv) at 0° C. under N₂ atmosphere. The resulting mixture was stirred for 1 h at 25° C. Then TEA (90.00 mg, 0.888 mmol, 3 equiv) was added into above system at 0° C. The resulting mixture was stirred for 1 h at 25° C. To the above mixture was added 1-methylcyclobutan-1-amine (50.49 mg, 0.592 mmol, 2 equiv) dropwise at 0° C. The reaction was stirred at 25° C. for 2 h. Quenched with water (10 mL) and extracted with DCM (3×10 mL). The combined extracts were washed with water, brine, dried over Na₂SO₄, filtered and concentrated. The residue was purified by preparative HPLC using a XBridge Shield RP18 OBD 30*150 mm, 5 μm column (eluent: 42% to 60% (v/v) CH₃CN and H₂O with 10 mmol/L NH₄HCO₃) to afford the title compound 1-[(2R)-2-methyl-3-oxo-4-{[6-(trifluoromethyl)pyridin-2-yl]methyl}-2H-1,4-benzoxazin-7-yl]-3-(1-methylcyclobutyl)urea (32.5 mg, 24.40%) as a white solid. MS (ESI): mass calcd. for C₂₂H₂₃F₃N₄O₃:448.17 m/z, found:449.15 [M+H]⁺. ¹H NMR (400 MHz, DMSO-d₆) δ 8.24 (s, 1H), 8.03-8.12 (m, 1H), 7.81 (d, J=7.7 Hz, 1H), 7.53 (d, J=8.0 Hz, 1H), 7.25 (d, J=2.0 Hz, 1H), 6.76-6.86 (m, 2H), 6.23 (s, 1H), 5.24 (s, 2H), 4.78-4.88 (m, 1H), 2.22-2.30 (m, 2H), 1.82-1.93 (m, 2H), 1.69-1.82 (m, 2H), 1.47 (d, J=6.7 Hz, 3H), 1.38 (s, 3H). ¹⁹F NMR (376 MHz, DMSO) δ −66.54.

The following examples in Table 12 were made according to the procedure for example 226.

TABLE 12

| Structure | Ex. | IUPAC Name | 1H NMR | LCMS |
|---|---|---|---|---|
| 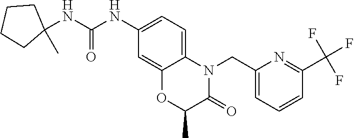 [SMILES: C[C@H]1Oc2cc(NC(=O)NC3(C)CCCC3)ccc2N(Cc2cccc(C(F)(F)F)n2)C1=O] | 227 | (R)-1-(2-methyl-3- oxo-4-((6- (trifluoromethyl) pyridin-2-yl)methyl)- 3,4-dihydro-2H- benzo[b][1,4]oxazin- 7-yl)-3-(1- methylcyclopentyl) urea | ¹H NMR (300 MHz, DMSO-d₆) δ 8.21 (s, 1H), 8.00-8.15 (m, 1H), 7.72-7.88 (m, 1H), 7.39-7.55 (m, 1H), 7.11-7.32 (m, 1H), 6.61-6.91 (m, 2H), 6.00 (s, 1H), 5.24 (s, 2H), 4.81 (q, J = | calcd. for C₂₃H₂₅F₃N₄O₃: 462.19 m/z, found:463.20 [M + H]⁺ |
| | | | 6.7 Hz, 1H), | |
| | | | 1.80-2.00 (m, 2H), | |
| | | | 1.58-1.71 (m, 4H), | |
| | | | 1.49-1.58 (m, 2H), | |
| | | | 1.41-1.49 (m, 3H), | |
| | | | 1.33 (s, 3H). 19F | |
| | | | NMR (282 MHZ, | |
| | | | DMSO) δ −66.55. | |
| 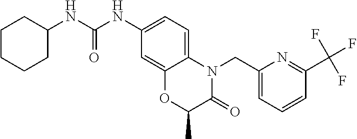 [SMILES: C[C@H]1Oc2cc(NC(=O)NC3CCCCC3)ccc2N(Cc2cccc(C(F)(F)F)n2)C1=O] | 228 | (R)-1-cyclohexyl- 3-(2-methyl-3-oxo- 4-((6- (trifluoromethyl)py ridin-2-yl)methyl)- 3,4-dihydro-2H- benzo[b][1,4]oxazin- 7-yl)urea | ¹H NMR (400 MHz, DMSO-d₆) δ 8.28 (s, 1H),8.07 (t, J = 7.9 Hz, 1H), 7.81 (d, J = 7.6 Hz, 1H), 7.53 (d, J = 8.0 Hz, 1H), 7Hz, 1H), 6.74-.26 (d, J = 1.9 6.92 (m, 2H), 6.03 | calcd. for C₂₃H₂₅F₃N₄O₃: 462.19, 463.05 [M + H]⁺ |
| | | | (d, J = 7.8 Hz, | |
| | | | 1H), 5.24 (d, J = | |
| | | | 3.4 Hz, 2H), 4.80 | |
| | | | (q, J = 6.7 Hz, | |
| | | | 1H),3.45 (m,1H), | |
| | | | 1.80 (d, J = 11.6 | |
| | | | Hz, 2H), 1.61- | |
| | | | 1.69 (m, 2H), 1.53 | |
| | | | (s, 4H), 1.28-1.40 | |
| | | | (m, 2H), 0.83- | |
| | | | 1.07 (m, 3H). 19F | |
| | | | NMR (376 MHZ, | |
| | | | DMSO) δ −66.54 | |
| 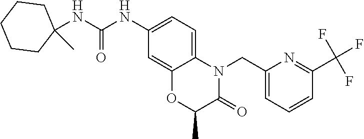 [SMILES: C[C@H]1Oc2cc(NC(=O)NC3(C)CCCCC3)ccc2N(Cc2cccc(C(F)(F)F)n2)C1=O] | 229 | (R)-1-(2-methyl-3- oxo-4-((6- (trifluoromethyl)py ridin-2-yl)methyl)- 3,4-dihydro-2H- benzo[b][1,4]oxazin- 7-yl)-3-(1- methylcyclohexyl) urea | ¹H NMR (400 MHz, DMSO-d₆) δ 8.32 (s, 1H), 8.07 (t, J = 7.9 Hz, 1H), 7.81 (d, J = 7.8 Hz, 1H), 7.53 (d, J = 8.0 Hz, 1H), 7.25 (d, J = 2.3 Hz, 1H), 6.76 | calcd. for C₂₄H₂₇F₃N₄O3: 476.20, found: 477.05 [M + H]⁺ |
| | | | (d, J = 8.7 Hz, | |
| | | | 2H), 5.79 (s, 1H), | |
| | | | 5.24 (s, 2H), 4.80 | |
| | | | (q, J = 6.7 Hz, | |
| | | | 1H), 1.92 (d, J = | |
| | | | 12.7 Hz, 2H), | |
| | | | 1.48-1.68 (m, 8H), | |
| | | | 1.44 -1.90 (m, | |
| | | | 6H). ¹⁹F NMR | |
| | | | (376 MHZ, | |
| | | | DMSO) δ −66.54 | |
| 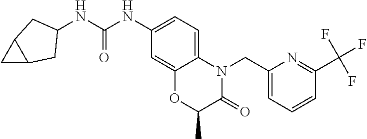 [SMILES: C[C@H]1Oc2cc(NC(=O)NC3CC4CC4C3)ccc2N(Cc2cccc(C(F)(F)F)n2)C1=O] | 230 | 1- (bicyclo[3.1.0]hexan- 3-yl)-3-((R)-2- methyl-3-oxo-4- ((6- (trifluoromethyl) pyridin-2-yl)methyl)- 3,4-dihydro-2H- benzo[b][1,4]oxazin- 7-yl)urea | ¹H NMR (400 MHz, DMSO-d₆) 8 8.18 (s, 1H), 7.99-8.12 (m, 1H), 7.70-7.92 (m, 1H), 7.40- 7.69(m, 1H), 7.14-7.31(m, 1H), 6.59- 7.00(m, 2H), | calcd. for C₂₃H₂₃F₃N₄O₃: 460.1 m/z, found: 461.10 [M + H]⁺. |
| | | | 5.78- 6.15 (m, | |
| | | | 1H), 5.05 - 5.35 | |
| | | | (m, 2H), 4.62 - | |
| | | | 4.96 (m, 1H), 3.92- | |
| | | | 4.25 (m, 1H), | |
| | | | 2.06-2.26 (m, | |
| | | | 2H), 1.38-1.59 | |
| | | | (m, 5H), 1.22- | |
| | | | 1.31 (m, 2H), 0.45- | |
| | | | 0.65 (m, 1H), | |
| | | | 0.17-0.28 (m, | |
| | | | 1H). ¹⁹F NMR (376 | |
| | | | MHz, DMSO) δ | |
| | | | −66.55. | |
| 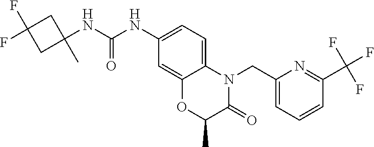 [SMILES: C[C@H]1Oc2cc(NC(=O)NC3(C)CC(F)(F)C3)ccc2N(Cc2cccc(C(F)(F)F)n2)C1=O] | 231 | (R)-1-(3,3-difluoro- 1- methylcyclobutyl)- 3-(2-methyl-3-oxo- 4-((6- (trifluoromethyl) pyridin-2-yl)methyl)- 3,4-dihydro-2H- benzo[b][1,4]oxazin- | ¹H NMR (400 MHz, DMSO-d₆) δ 8.39 (s, 1H), 7.95-8.21 (m, 1H), 7.81 (d, J = 7.7 Hz, 1H), 7.42-7.68 (m, 1H), 7.20 (d, J = 1.3 Hz, 1H), 6.84 (d, J = 1.4 | calcd. for C₂₂H₂₁F₅N₄O₃: 484.15, found: 484.95 [M + H]⁺ |
| | | 7-yl)urea | Hz, 2H), 6.51 (s, | |
| | | | 1H), 5.24 (s, 2H), | |
| | | | 4.59-4.99 (m, 1H), | |
| | | | 2.78-3.02 (m, 2H), | |
| | | | 2.55-2.68 (m, 2H), | |
| | | | 1.39-1.59 (m, 6H). | |
| | | | 19F NMR (376 | |
| | | | MHz, DMSO) δ | |
| | | | −66.55, −85.05, | |
| | | | −89.67 | |
| 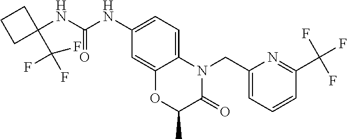 [SMILES: C[C@H]1Oc2cc(NC(=O)NC3(C(F)(F)F)CCC3)ccc2N(Cc2cccc(C(F)(F)F)n2)C1=O] | 232 | (R)-1-(2-methyl-3- oxo-4-((6- (trifluoromethyl) pyridin-2-yl)methyl)- 3,4-dihydro-2H- benzo[b][1,4]oxazin- 7-yl)-3-(1- (trifluoromethyl) cyclobutyl)urea | ¹H NMR (300 MHz, DMSO-d₆) δ 8.38 (s, 1H), 8.07 (t, J = 7.8 Hz, 1H), 7.81 (d, J = 7.8 Hz, 1H), 7.53 (d, J = 7.8 Hz, 1H), 7.24 (s, 1H), 6.81 (d, J = 15.3 | calcd. for C22H20F6N403: 502.1 m/z, found: 503.30 [M + H]⁺ |
| | | | Hz, 3H), 5.24 (s, | |
| | | | 2H), 4.84 (q, J = | |
| | | | 6.6 Hz, 1H), 2.40 | |
| | | | (t, J = 7.4 Hz, 4H), | |
| | | | 1.97 - 1.90 (m, | |
| | | | 2H), 1.47 (d, J = | |
| | | | 6.6 Hz, 3H).19F | |
| | | | NMR (282 MHZ, | |
| | | | DMSO-d₆) δ | |
| | | | −66.55, −79.85. | |
| 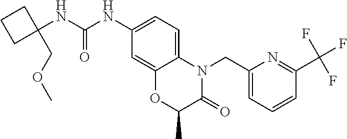 [SMILES: COCC1(NC(=O)Nc2ccc3c(c2)O[C@H](C)C(=O)N3Cc2cccc(C(F)(F)F)n2)CCC1] | 233 | (R)-1-(1- (methoxymethyl) cyclobutyl)-3-(2- methyl-3-oxo-4- ((6- (trifluoromethyl) pyridin-2-yl)methyl)- 3,4-dihydro-2H- benzo[b][1,4]oxazin- | ¹H NMR (300 MHz, DMSO-d₆) δ 8.41 (s, 1H), 8.07 (t, J = 7.8 Hz, 1H), 7.81 (d, J = 7.8 Hz, 1H), 7.52 (d, J = 8.1 Hz, 1H), 7.25 (d, J = 2.1 Hz, 1H), 6.84- | calcd. for C₂₃H₂₅F₃N₄O₄: 478.2 m/z, found:479.15 [M + H]⁺ |
| | | 7-yl)urea | 6.79 (m, 2H), | |
| | | | 6.19 (s, 1H), 5.24 | |
| | | | (s, 2H), 4.83 (q, J = | |
| | | | 6.6 Hz, 1H), | |
| | | | 3.47 (s, 2H), 3.30 | |
| | | | (s, 3H), 2.24 (q, J = | |
| | | | 9.9 Hz, 2H), | |
| | | | 1.99-1.92 (m, | |
| | | | 2H),1.79-1.72 | |
| | | | (m, 2H), 1.47 (d, J = | |
| | | | 6.6 Hz, 3H).19F | |
| | | | NMR (282 MHZ, | |
| | | | DMSO-d₆) δ | |
| | | | −66.18. | |
| 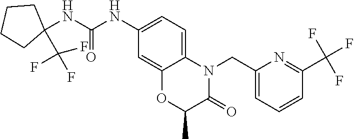 [SMILES: C[C@H]1Oc2cc(NC(=O)NC3(C(F)(F)F)CCCC3)ccc2N(Cc2cccc(C(F)(F)F)n2)C1=O] | 234 | (R)-1-(2-methyl-3- oxo-4-((6- (trifluoromethyl) pyridin-2-yl)methyl)- 3,4-dihydro-2H- benzo[b][1,4]oxazin- 7-yl)-3-(1- (trifluoromethyl) cyclopentyl)urea | ¹H NMR (300 MHz, DMSO-d₆) δ 8.35 (s, 1H), 8.07 (t, J = 7.8 Hz, 1H), 7.81 (d, J = 7.5 Hz, 1H), 7.53 (d, J = 8.1 Hz, 1H), 7.23 (d, J = 1.8 Hz, 1H), 6.85- | calcd. for C₂₃H₂₂F₆N₄O₃: 516.2 m/z, found: 517.15 [M + H]⁺ |
| | | | 6.77 (m, 2H), | |
| | | | 6.49 (s, 1H), 5.24 | |
| | | | (s, 2H), 4.84 (q, J = | |
| | | | 6.6 Hz, 1H), | |
| | | | 2.26-2.22 (m, | |
| | | | 2H), 1.93-1.86 | |
| | | | (m, 2H), 1.80- | |
| | | | 1.60 (m, 4H), 1.47 | |
| | | | (d, J = 6.6 Hz, | |
| | | | 3H).¹⁹F NMR (282 | |
| | | | MHz, DMSO-d₆) | |
| | | | δ −66.56, −75.84. | |
| 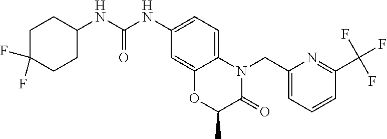 [SMILES: C[C@H]1Oc2cc(NC(=O)NC3CCC(F)(F)CC3)ccc2N(Cc2cccc(C(F)(F)F)n2)C1=O] | 235 | (R)-1-(4,4- difluorocyclohexyl)- 3-(2-methyl-3- oxo-4-((6- (trifluoromethyl)py ridin-2-yl)methyl)- 3,4-dihydro-2H- benzo[b][1,4]oxazin- 7-yl)urea | ¹H NMR (400MHz, DMSO-d₆) δ 8.30 (s, 1H), 8.02-8.22 (m, 1H), 7.75-7.91 (m, 1H), 7.42-7.58 (m, 1H), 7.18-7.32 (m, 1H), 6.85 (s, 2H), 6.12-6.38 (m, | calcd. for C₂₃H₂₃F5N₄O₃: 498.17 m/z, found: 499.05 [M + H]⁺ |
| | | | 1H), 5.18-5.37(m, | |
| | | | 2H), 4.68-4.91(m, | |
| | | | 1H), 3.69(s, 1H), | |
| | | | 1.81-2.10 (m, 6H), | |
| | | | 1.41-1.58 (m, 5H) | |
| | | | ¹⁹F NMR (376 | |
| | | | MHz, DMSO) δ - | |
| | | | −66.59 | |

### Example 236

### (R)-1-(4-benzyl-2-methyl-3-oxo-3,4-dihydro-2H-benzo[b][1,4]oxazin-7-yl)-3-(4-hydroxy-2-methylbutan-2-yl)urea

### Synthetic Scheme

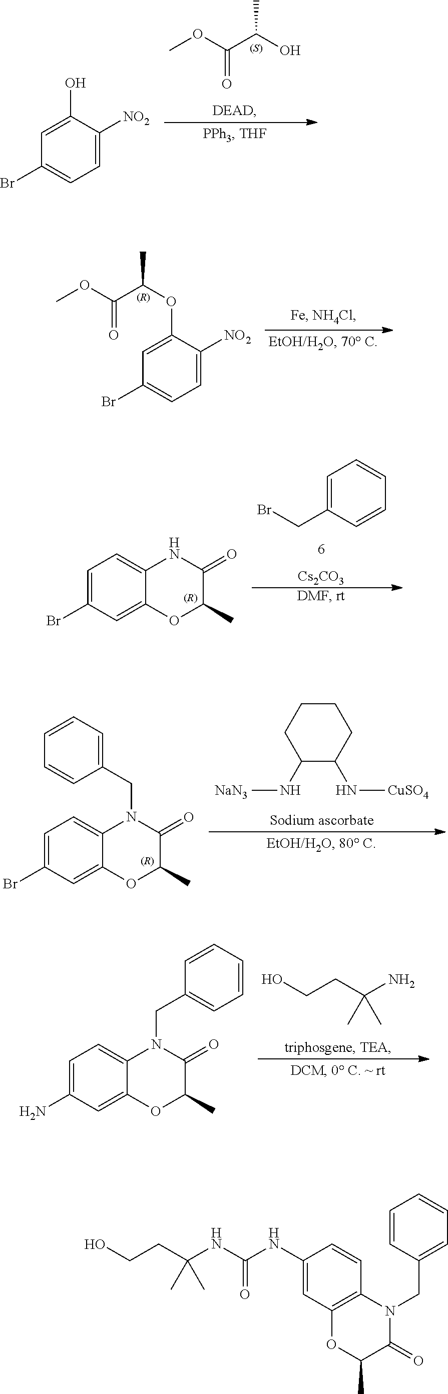

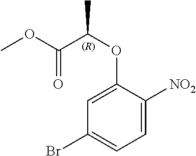

### methyl (R)-2-(5-bromo-2-nitrophenoxy)propanoate

To a solution of 2-amino-5-nitrophenol (900 mg, 0.065 mmol, 1 equiv) in THF (15 mL) was added methyl (2S)-2-hydroxypropanoate (649.68 mg, 6.240 mmol, 1.5 equiv) and PPh₃ (1636.87 mg, 6.240 mmol, 1.5 equiv). The resulting mixture was maintained under nitrogen and stirred at rt for 1 h. Then DEAD (1086.85 mg, 6.240 mmol, 1.5 equiv) was added at 0° C. The resulting mixture was maintained under nitrogen and stirred at 80° C. for 3 h. After cooling down to rt, the reaction was quenched with water (100 mL). The resulting mixture was extracted with ethyl acetate (3×100 mL). The organic layers were combined, dried over anhydrous sodium sulfate, filtered and concentrated. The residue obtained was purified by silica gel chromatography (0-20% ethyl acetate/petroleum ether) to afford the methyl (2R)-2-(5-bromo-2-nitrophenoxy)propanoate (1.12 g, 88.53%) as a yellow oil. LC/MS: mass calcd. for C₁₀H₁₀BrNO₅: 302.97, found: 304.09, 306.09 [M+H, M+H+2]⁺.

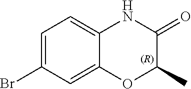

### (R)-7-bromo-2-methyl-2H-benzo[b][1,4]oxazin-3(4H)-one

To a solution of methyl (2R)-2-(5-bromo-2-nitrophenoxy)propanoate (1.1 g, 3.63 mmol, 1 equiv) in MeOH (15 mL) and H₂O (1.5 mL) was added Fe (2.03 g, 36.3 mmol, 10 equiv) and NH₄C₁ (1.96 g, 36.3 mmol, 10 equiv). The resulting mixture was maintained under nitrogen and stirred at 70° C. for 3 h. After cooling down to rt, the reaction was quenched with water (50 mL). The resulting mixture was extracted with EA (3×50 mL). The organic layers were combined, dried over anhydrous sodium sulfate, filtered and concentrated. The residue obtained was purified by silica gel chromatography (0-100% ethyl acetate/petroleum ether) to afford the methyl (2R)-2-(5-bromo-2-nitrophenoxy)propanoate (800 mg, 91.53%) as a yellow oil. LC/MS: mass calcd. for C₉H₈BrNO₂: 240.97, found: 241.07, 243.97 [M+H, M+H+2]⁺.

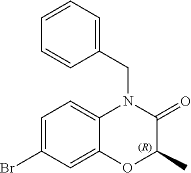

### (R)-4-benzyl-7-bromo-2-methyl-2H-benzo[b][1,4]oxazin-3(4H)-one

To a solution of (2R)-7-bromo-2-methyl-2,4-dihydro-1,4-benzoxazin-3-one (2 g, 8.262 mmol, 1 equiv) and benzyl bromide (2826.22 mg, 16.524 mmol, 2 equiv) in dimethylformamide (50 mL) was added Cs₂CO₃ (8100.74 mg, 24.786 mmol, 3 equiv). The reaction was stirred at 100° C. under N₂ for 3 h. After cooling down to rt, the reaction was quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give (2R)-4-benzyl-7-bromo-2-methyl-2H-1,4-benzoxazin-3-one (2.35 g, 85.62%) as a white solid. LC/MS (ESI): mass calcd. for C₁₆H₁₄BrNO₂: 331.02 m/z, found:332.00, 334.00 [M+H, M+H+2]⁺.

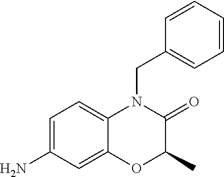 [SMILES: C[C@H]1Oc2cc(N)ccc2N(Cc2ccccc2)C1=O]

### (R)-7-amino-4-benzyl-2-methyl-2H-benzo[b][1,4]oxazin-3(4H)-one

To a solution of (2R)-4-benzyl-7-bromo-2-methyl-2H-1,4-benzoxazin-3-one (2.35 g, 5.916 mmol, 1 equiv) in ethyl alcohol (60 mL) and water (15 mL) was added N₁,N₂-dimethylcyclohexane-1,2-diamine (0.50 g, 3.550 mmol, 0.6 equiv), CuSO₄ (0.94 g, 5.916 mmol, 1 equiv) and NaN₃ (1.15 g, 17.75 mmol, 3 equiv). The reaction was stirred at 80° C. under N₂ for 3 h. After cooling down to rt, the reaction was quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give (2R)-7-amino-4-benzyl-2-methyl-2H-1,4-benzoxazin-3-one (1.3 g, 81.89%) as a white solid. LC/MS (ESI): mass calcd. for C₁₆H₁₆N₂O₂: 268.12 m/z, found:269.15 [M+H]⁺.

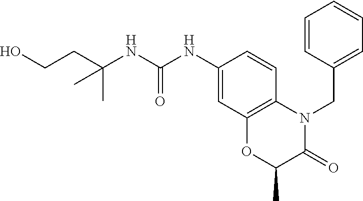 [SMILES: C[C@H]1Oc2cc(NC(=O)NC(C)(C)CCO)ccc2N(Cc2ccccc2)C1=O]

### (R)-1-(4-benzyl-2-methyl-3-oxo-3,4-dihydro-2H-benzo[b][1,4]oxazin-7-yl)-3-(4-hydroxy-2-methylbutan-2-yl)urea

To a solution of (2R)-7-amino-4-benzyl-2-methyl-2H-1,4-benzoxazin-3-one (100 mg, 0.373 mmol, 1 equiv) in DCM (5 mL) was added triphosgene (44.24 mg, 0.149 mmol, 0.4 equiv). The reaction was stirred at 0° C. under N₂ for 1 h. The reaction was added TEA (188.57 mg, 1.865 mmol, 5 equiv) and stirred at rt under N₂ for 1 h. The reaction was added 3-amino-3-methylbutan-1-ol (153.80 mg, 1.492 mmol, 4 equiv) and stirred at rt under N₂ for 1 h. Quenched with water (20 mL) and extracted with DCM (3×20 mL). The combined extracts were washed with water, brine, dried over Na₂SO₄, filtered and concentrated. The residue was purified by preparative HPLC using a XBridge Prep Phenyl OBD Column 19*150 mm, 5 μm (eluent: 27% to 49% (v/v) CH₃CN and H₂O with 10 mmol/L NH₄HCO₃) to afford 1-[(2R)-4-benzyl-2-methyl-3-oxo-2H-1,4-benzoxazin-7-yl]-3-(4-hydroxy-2-methylbutan-2-yl)urea (36.2 mg, 24.36%) as a white solid. LC/MS (ESI): mass calcd. for C₂₂H₂₇N₃O₄:397.20 m/z, found:398.00 [M+H]⁺. ¹H NMR (300 MHz, DMSO-d₆) δ 8.27 (s, 1H), 7.26-7.38 (m, 2H), 7.20-7.25 (m, 4H), 6.75-6.86 (m, 2H), 5.95 (s, 1H), 5.09 (s, 2H), 4.79-4.81 (m, 1H), 4.37-4.41 (m, 1H), 3.44-3.50 (m, 2H), 1.78-1.80 (t, J=6 Hz, 2H), 1.46-1.48 (m, 3H), 1.25 (s, 6H).

The following examples in Table 13 were made according to the procedure for example 236.

TABLE 13

| Structure | Ex. | IUPAC Name | 1H NMR | LCMS |
|---|---|---|---|---|
| 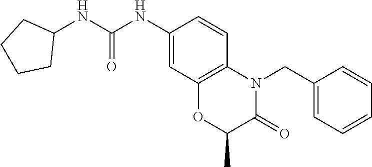 [SMILES: C[C@H]1Oc2cc(NC(=O)NC3CCCC3)ccc2N(Cc2ccccc2)C1=O] | 237 | (R)-1-(4-benzyl-2- methyl-3-oxo-3,4- dihydro-2H- benzo[b][1,4] oxazin-7-yl)-3- cyclopentylurea | ¹H NMR (300 MHZ, DMSO-d₆) δ 8.22 (s, 1H), 7.35-7.30 (m, 2H), 7.25-7.22 (m, 4H), 6.86 (d, J = 8.7 Hz, 1H), 6.80-6.77 (m, 1H), 6.12 (d, J = 7.2 Hz, 1H), 5.09 (d, J = 2.7 Hz, 2H), 4.82 (q, J = 6.6 Hz, 1H), | calcd. for C₂₂H₂₅N₃O₃: 379.2 m/z, found: 380.15 [M + H]⁺. |
| | | | 3.89 (q, J = 6.6 Hz, | |
| | | | 1H), 1.83-1.77 (m, | |
| | | | 2H), 1.61-1.35 (m, | |
| | | | 7H), 1.33-1.29 (m, | |
| | | | 2H). | |
| 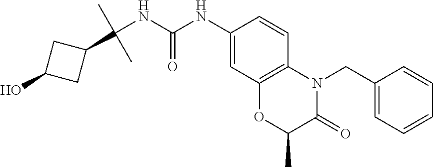 [SMILES: C[C@H]1Oc2cc(NC(=O)NC(C)(C)[C@H]3C[C@@H](O)C3)ccc2N(Cc2ccccc2)C1=O] | 238 | 1-((R)-4-benzyl-2- methyl-3-oxo-3,4- dihydro-2H- benzo[b][1,4] oxazin-7-yl)-3- (2- ((1s,3S)-3- hydr- oxycyclobutyl propan-2-yl)urea | ¹H NMR (400 MHZ, DMSO-d₆) δ 8.10- 8.52 (m, 1H), 7.10- 7.44 (m, 6H), 6.80- 6.97 (m, 1H), 6.60- 6.80 (m, 1H), 5.56- 5.91 (m, 1H), 5.03- 5.22 (m, 2H), 4.90- 5.03 (m, 1H), 4.63- 4.90 (m, 1H), 3.6 - 3.93 (m, 1H), 1.83- | calcd. for C₂₄H₂₉N₃O₄: 423.22 m/z, found 424.05 [M + H]⁺ |
| | | | 2.15 (m, 4H), 1.53- | |
| | | | 1.69 (m, 2H), 1.35- | |
| | | | 1.53 (m, 3H), 1.00- | |
| | | | 1.30 (m, 6H). | |

### Example 239: 1-(2-hydroxy-2-methylpropyl)-3-(2-oxo-1-(3-(trifluoromethoxy)benzyl)-1,2-dihydroquinoxalin-6-yl)urea

### Synthetic Scheme

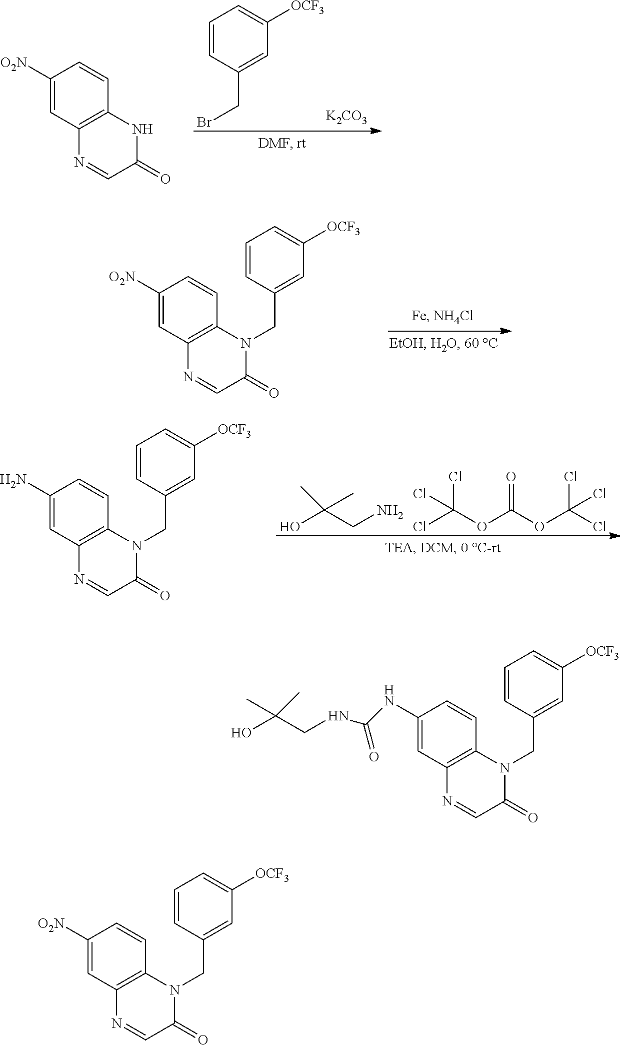

### 6-nitro-1-(3-(trifluoromethoxy)benzyl)quinoxalin-2(1H)-one

To a mixture of 6-nitroquinoxalin-2(1H)-one (1.8 g, 9.417 mmol, 1.2 equiv) and 1-(bromomethyl)-3-(trifluoromethoxy)benzene (2.00 g, 7.848 mmol, 1 equiv) in DMF (30 mL) was stirred and added K₂CO₃ (2.18 g, 15.695 mmol, 2 equiv). The reaction was stirred at rt for 1 h. The reaction mixture was quenched by ice water (30 mL) and extracted with EA (3×30 mL). The organic layers were combined, dried over anhydrous sodium sulfate, filtered and concentrated under reduced pressure. The residue was purified by silica gel column, eluted with PE/EA (3:1) to afford 6-nitro-1-(3-(trifluoromethoxy)benzyl)quinoxalin-2(1H)-one (1.7 g, 59.31%) as a yellow solid. MS (ESI): mass calcd. for C₁₆H₁₀F₃N₃O₄, 365.06 m/z, found 366.00 [M+H]⁺.

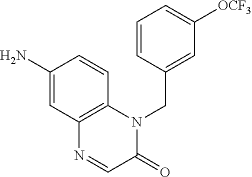 [SMILES: Nc1ccc2c(c1)ncc(=O)n2Cc1cccc(OC(F)(F)F)c1]

### 6-amino-1-(3-(trifluoromethoxy)benzyl)quinoxalin-2(1H)-one

To a mixture of 6-nitro-1-(3-(trifluoromethoxy)benzyl)quinoxalin-2(1H)-one (1.0 g, 2.738 mmol, 1 equiv) in EtOH (25 mL) and water (5 mL). Fe (1.53 g, 27.380 mmol, 10 equiv) and NH₄Cl (1.46 g, 27.380 mmol, 10 equiv) was added into the mixture. The resulting solution was stirred at 60° C. for 1 h. The reaction was quenched with water (30 mL) at rt. After filtration, the filter cake was washed with EA (3×20 mL). The filtrate was added water (20 mL) and extracted with EA (3×50 mL). The combined organic layers were washed with brine (30 mL), dried over anhydrous Na₂SO₄. After filtration, the filtrate was concentrated under reduced pressure. The crude product was purified by silica gel column chromatography, eluted with PE/EA (1:1) to afford 6-amino-1-(3-(trifluoromethoxy)benzyl)quinoxalin-2(1H)-one (700 mg, 75.51%) as a yellow solid. MS (ESI): mass calcd. for C₁₆H₁₂F₃N₃O₂, 335.09 m/z, found 336.05 [M+H]⁺.

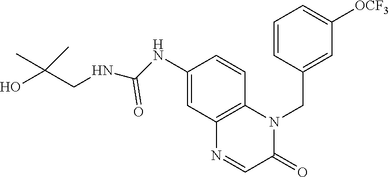 [SMILES: CC(C)(O)CNC(=O)Nc1ccc2c(c1)ncc(=O)n2Cc1cccc(OC(F)(F)F)c1]

### 1-(2-hydroxy-2-methylpropyl)-3-(2-oxo-1-(3-(trifluoromethoxy)benzyl)-1,2-dihydroquinoxalin-6-yl)urea

To a mixture of 6-amino-1-(3-(trifluoromethoxy)benzyl)quinoxalin-2(1H)-one (70 mg, 0.209 mmol, 1 equiv) in DCM (6 mL) was stirred under nitrogen atmosphere and added triphosgene (19 mg, 0.063 mmol, 0.3 equiv) at 0° C. The reaction was stirred at rt for 1 h. TEA (53 mg, 0.522 mmol, 2.5 equiv) was added into the mixture at 0° C. The reaction was stirred at rt for 1 h. 1-amino-2-methylpropan-2-ol (23 mg, 0.251 mmol, 1.2 equiv) was added into the mixture at 0° C. The reaction was stirred at rt for 1 h. The reaction mixture was quenched by 1N hydrochloric acid (5 mL) and extracted with DCM (3×10 mL). The organic layers were combined, dried over anhydrous sodium sulfate, filtered and concentrated under vacuum. The crude product was purified by silica gel column chromatography, eluted with PE/EA (1:1) to afford 1-(2-hydroxy-2-methylpropyl)-3-(2-oxo-1-(3-(trifluoromethoxy)benzyl)-1,2-dihydroquinoxalin-6-yl)urea (26.1 mg, 27.71%) as a yellow solid. MS (ESI): mass calcd. for C₂₁H₂₁F₃N₄O₄, 450.15 m/z, found 451.00 [M+H]+. ¹H NMR (300 MHz, DMSO-d₆) δ 8.86 (s, 1H), 8.33 (s, 1H), 8.01 (d, J=2.4 Hz, 1H), 7.41-7.53 (m, 2H), 7.32-7.41 (m, 2H), 7.21-7.32 (m, 2H), 6.18 (t, J=5.7 Hz, 1H), 5.49 (s, 2H), 4.55 (s, 1H), 3.05 (d, J=5.7 Hz, 2H), 1.09 (s, 6H).

The following examples in Table 14 were made according to the procedure for example 239.

TABLE 14

| Structure | Ex. | IUPAC Name | 1H NMR | LCMS |
|---|---|---|---|---|
| 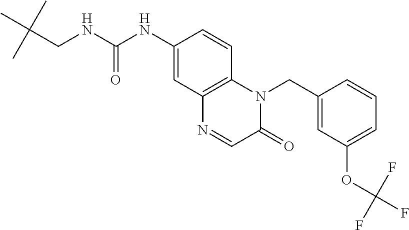 [SMILES: CC(C)(C)CNC(=O)Nc1ccc2c(c1)ncc(=O)n2Cc1cccc(OC(F)(F)F)c1] | 240 | 1-neopentyl-3-(2- oxo-1-(3- (trifluoromethoxy) benzyl)-1,2- dihydroquinoxalin- 6-yl)urea | ¹H NMR (400 MHZ, DMSO-d₆) δ 8.63 (s, 1H), 8.32 (s, 1H), 8.02 (d, J = 2.5 Hz, 1H), 7.43-7.50(m, 2H), 7.32-7.40 (m, 2H), 7.26 (dd, J = 8.8, 7.6 Hz, 2H), 6.24 (t, J = 6.2 Hz, 1H), 5.50 (s, 2H), 2.92 (d, J = 6.1 Hz, 2H), 0.87 (s, 9H). | calcd. for C₂₂H₂₃F₃N₄O₃, 448.17 m/z, found 448.95 [M + H]⁺ |
| 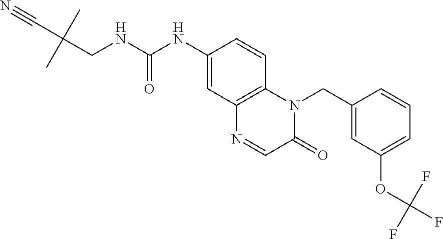 [SMILES: CC(C)(C#N)CNC(=O)Nc1ccc2c(c1)ncc(=O)n2Cc1cccc(OC(F)(F)F)c1] | 241 | 1-(2-cyano-2- methylpropyl)-3-(2- oxo-1-(3- (trifluoromethoxy) benzyl)-1,2- dihydroquinoxalin- 6-yl)urea | ¹H NMR (300 MHZ, DMSO-d₆) δ 8.82 (s, 1H), 8.33 (s, 1H), 8.03 (d, J = 2.4 Hz, 1H), 7.33-7.53 (m, 4H), 7.26 (t, J = 7.0 Hz, 2H), 6.64 (t, J = 6.5 Hz, 1H), 5.50 (s, 2H), 3.31 (d, J = 6.6 Hz, 2H), 1.29 (s, 6H). | calcd. for C₂₂H₂₀F₃N₅O₃, 459.15 m/z, found 460.05 [M + H]⁺ |
| 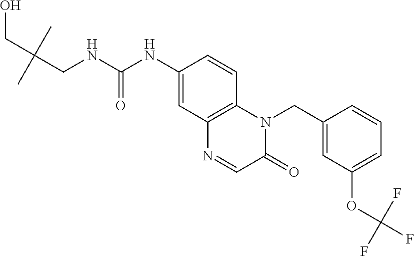 [SMILES: CC(C)(CO)CNC(=O)Nc1ccc2c(c1)ncc(=O)n2Cc1cccc(OC(F)(F)F)c1] | 242 | 1-(3-hydroxy-2,2- dimethylpropyl)-3- (2-oxo-1-(3- (trifluoromethoxy) benzyl)-1,2- dihydroquinoxalin- 6-yl)urea | ¹H NMR (300 MHZ, DMSO-d₆) δ 8.75 (s, 1H), 8.32 (s, 1H), 8.00 (d, J = 2.4 Hz, 1H), 7.41 - 7.51 (m, 2H), 7.36 (d, J = 9.4 Hz, 2H), 7.26 (t, J = 7.2 Hz, 2H), 6.23 (t, J = 6.1 Hz, 1H), 5.49 (s, 2H), 4.63 (t, J = 5.5 Hz, 1H), 3.13 (d, J = 5.5 Hz, 2H), 2.98 (d, J = 6.0 Hz, 2H), 0.79 (s, 6H). | calcd. for C₂₂H₂₃F₃N₄O₄, 464.17 m/z, found 465.05 [M + H]⁺ |
| 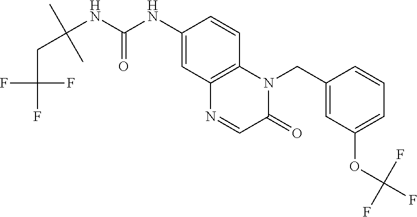 [SMILES: CC(C)(CC(F)(F)F)NC(=O)Nc1ccc2c(c1)ncc(=O)n2Cc1cccc(OC(F)(F)F)c1] | 243 | 1-(2-oxo-1-(3- (trifluoromethoxy) enzyl)-1,2- dihydroquinoxalin- 6-yl)-3-(4,4,4- trifluoro-2- methylbutan-2- yl)urea | ¹H NMR (300 MHZ, DMSO-d₆) δ 8.54 (s, 1H), 8.33 (s, 1H), 7.98 (d, J = 2.4 Hz, 1H), 7.41 - 7.48 (m, 2H), 7.36 (d, J = 9.1 Hz, 2H), 7.26 (t, J = 7.8 Hz, 2H), 6.26 (s, 1H), 5.49 (s, 2H), 2.84 (q, J = 12.1 Hz, 2H), 1.37 (s, 6H). | calcd. for C₂₂H₂₀F₆N₄O₃, 502.14 m/z, found 503.05 [M + H]⁺ |
| 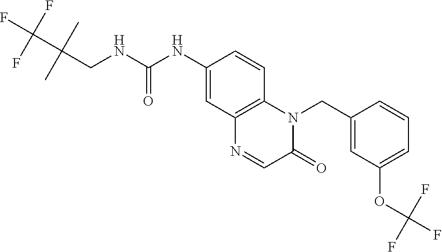 [SMILES: CC(C)(CNC(=O)Nc1ccc2c(c1)ncc(=O)n2Cc1cccc(OC(F)(F)F)c1)C(F)(F)F] | 244 | 1-(2-oxo-1-(3- (trifluoromethoxy) benzyl)-1,2- dihydroquinoxalin- 6-yl)-3-(3,3,3- trifluoro-2,2- dimethylpropyl) urea | ¹H NMR (300 MHZ, DMSO-d₆) δ 8.80 (s, 1H), 8.33 (s, 1H), 8.03 (d, J = 2.4 Hz, 1H), 7.41-7.55 (m, 2H), 7.32-7.41 (m, 2H), 7.21-7.31 (m, 2H), 6.41 (t, J = 6.5 Hz, 1H), 5.50 (s, 2H), 3.30 (d, J = 6.4 Hz, 2H), 1.08 (s, 6H). | calcd. For C₂₂H₂₀F₆N₄O₃: 502.14 m/z, found: 503.10 [M + H]⁺ |
| 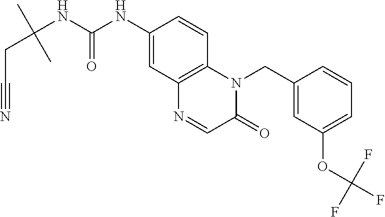 [SMILES: CC(C)(CC#N)NC(=O)Nc1ccc2c(c1)ncc(=O)n2Cc1cccc(OC(F)(F)F)c1] | 245 | 1-(1-cyano-2- methylpropan-2-yl)- 3-(2-oxo-1-(3- (trifluoromethoxy) enzyl)-1,2- dihydroquinoxalin- 6-yl)urea | ¹H NMR (400 MHZ, DMSO-d₆) δ 8.62 (s, 1H), 8.33 (s, 1H), 8.00 (d, J = 2.5 Hz, 1H), 7.42-7.50 (m, 2H), 7.33-7.42 (m, 2H), 7.26 (t, J = 8.1 Hz, 2H), 6.42 (s, 1H), 5.50 (s, 2H), 3.09 (s, 2H), 1.36 (s, 6H). | calcd. For C₂₂H₂₀F₃N₅O₃: 459.15 m/z, found: 460.10 [M + H]⁺ |

### Example 246: 1-(tert-butyl)-3-(1-(1-(3-cyclobutylphenyl)ethyl)-2-oxo-1,2-dihydroquinoxalin-6-yl)urea

### Synthetic Scheme

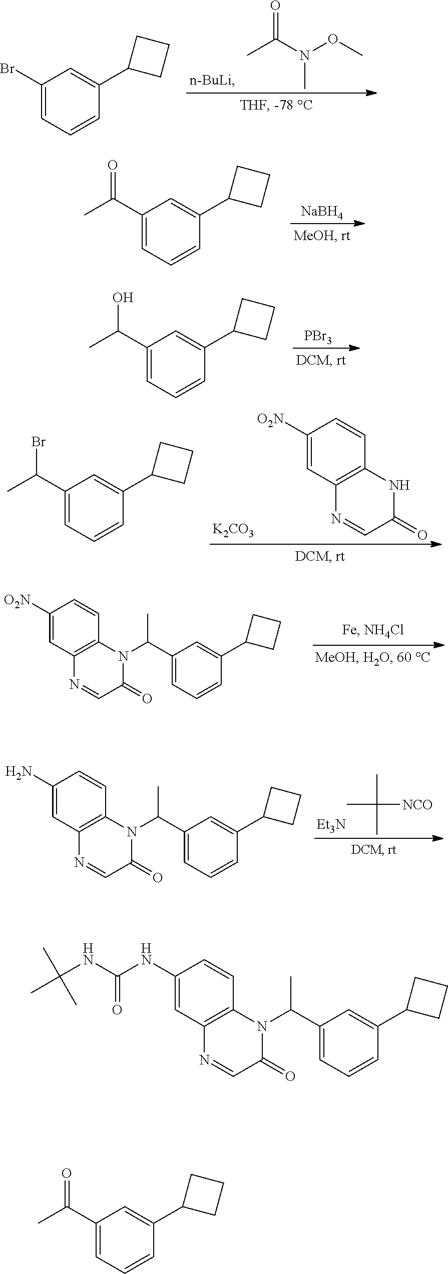

### 1-(3-cyclobutylphenyl)ethan-1-one

In a 100-mL round bottom flask, to a solution of 1-bromo-3-cyclobutylbenzene (500 mg, 2.369 mmol, 1 equiv) in tetrahydrofuran (5 mL) was added dropwise n-BuLi (2.5 M, 1.1 mL, 2.843 mmol, 1.2 equiv) at −78° C. under N₂ atmosphere. The reaction mixture was stirred at −78° C. for 30 mins. N-methoxy-N-methylacetamide (292 mg, 2.843 mmol, 1.2 equiv) was added into the solution and the mixture was stirred for another 1 h. The reaction was quenched with water/sat. NH₄Cl (5 mL), and then the mixture was extracted with ether/EtOAc (2×15 mL). The combined organic extracts were washed with brine (10 mL), dried over anhydrous Na₂SO₄ and concentrated under vacuum. The residue was purified by silica gel column chromatography, eluted with PE/EA (5/1) to afford 1-(3-cyclobutylphenyl)ethan-1-one (280 mg, 67.96%) as a white solid. MS (ESI): mass calcd. for C₁₂H₁₄O, 174.10 m/z, found 175.20 [M+H]⁺.

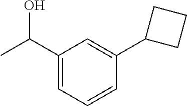

### 1-(3-cyclobutylphenyl)ethan-1-ol

A solution of 1-(3-cyclobutylphenyl)ethanone (280 mg, 1.607 mmol, 1 equiv) in MeOH (5 mL) was added NaBH₄(152 mg, 4.018 mmol, 2.5 equiv) at rt. The resulting mixture was stirred for 1 h at rt. The reaction mixture was quenched by water (10 mL) and extracted with EA (3×10 mL). The combined organic extracts were washed with brine (10 mL), dried over anhydrous Na₂SO₄ and concentrated under vacuum. The crude product was purified by flash chromatography with PE/EA (3:1) to afford 1-(3-cyclobutylphenyl)ethan-1-ol (200 mg, 70.61%) as a white solid. MS: mass calcd. for C₁₂H₁₆O: 176.12, found: 159.15 [M-OH]⁺.

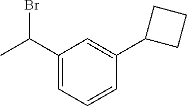

### 1-(1-bromoethyl)-3-cyclobutylbenzene

A solution of 1-(3-cyclobutylphenyl)ethan-1-ol (40 mg, 0.227 mmol, 1 equiv) in DCM (5 mL) was added phosphorus tribromide (154 mg, 0.568 mmol, 2.5 equiv) at rt. The resulting mixture was stirred for overnight at rt. The reaction mixture was quenched by ice-water (10 mL) and extracted with EA (3×10 mL). The combined organic extracts were washed with brine (10 mL), dried over anhydrous Na₂SO₄ and concentrated under vacuum. The crude product 1-(1-bromoethyl)-3-cyclobutylbenzene (40 mg, 73.70%) was used in the next step directly without further purification.

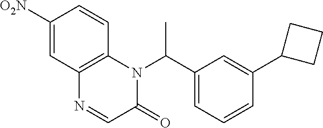 [SMILES: CC(c1cccc(C2CCC2)c1)n1c(=O)cnc2cc([N+](=O)[O-])ccc21]

### 1-(1-(3-cyclobutylphenyl)ethyl)-6-nitroquinoxalin-2(1H)-one

A solution of 1-(1-bromoethyl)-3-cyclobutylbenzene (150 mg, 0.627 mmol, 1 equiv) in dimethylformamide (5 mL) was added 6-nitroquinoxalin-2(1H)-one (239 mg, 1.254 mmol, 2.0 equiv) and K₂CO₃ (260 mg, 1.881 mmol, 3.0 equiv) at rt. The resulting mixture was stirred for overnight at rt. The reaction mixture was quenched by water (10 mL) The resulting mixture was extracted with EA (3×10 mL). The combined organic layers were washed with NaCl (aq., 3×5 mL), dried over anhydrous Na₂SO₄. After filtration, the filtrate was concentrated under reduced pressure. The residue was purified by silica gel column chromatography, eluted with PE/EA (⅓) to afford 1-(1-(3-cyclobutylphenyl)ethyl)-6-nitroquinoxalin-2(1H)-one (50 mg, 23.00%) as a light yellow solid. MS (ESI): mass calcd. C₂₀H₁₉N₃O₃ for: 349.14 m/z, found 350.20 [M+H]⁺.

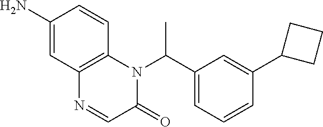 [SMILES: CC(c1cccc(C2CCC2)c1)n1c(=O)cnc2cc(N)ccc21]

### 6-amino-1-(1-(3-cyclobutylphenyl)ethyl)quinoxalin-2(1H)-one

A solution of 1-(1-(3-cyclobutylphenyl)ethyl)-6-nitroquinoxalin-2(1H)-one (60 mg, 0.172 mmol, 1 equiv) in EtOH (5 mL)/H₂O (0.5 mL) was added Fe (96 mg, 1.720 mmol, 10 equiv) and NH₄C₁ (92 mg, 1.720 mmol, 10 equiv) at rt. The resulting mixture was stirred for 1 h at 60° C. The mixture was allowed to cool down to rt. The reaction mixture was quenched by water (10 mL) and extracted with DCM (3×10 mL). The combined organic extracts were washed with brine (10 mL), dried over anhydrous Na₂SO₄ and concentrated under vacuum. The crude product was purified by flash chromatography with PE/EA (3:1) to afford 6-amino-1-(1-(3-cyclobutylphenyl)ethyl)quinoxalin-2(1H)-one (45 mg, 82.04%) as a light yellow solid. MS: mass calcd. for C₂₀H₂₁N₃O: 319.17, found: 320.20 [M+H]⁺.

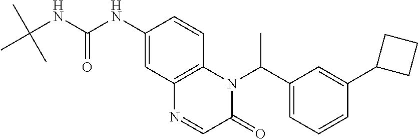 [SMILES: CC(c1cccc(C2CCC2)c1)n1c(=O)cnc2cc(NC(=O)NC(C)(C)C)ccc21]

### 1-(tert-butyl)-3-(1-(1-(3-cyclobutylphenyl)ethyl)-2-oxo-1,2-dihydroquinoxalin-6-yl)urea

A solution of 6-amino-1-(1-(3-cyclobutylphenyl)ethyl)quinoxalin-2(1H)-one (40 mg, 0.125 mmol, 1 equiv) in DCM (5 mL) was added 2-isocyanato-2-methylpropane (37 mg, 0.375 mmol, 3.0 equiv) and triethylamine (51 mg, 0.500 mmol, 4.0 equiv) at rt. The resulting mixture was stirred for overnight at rt. The reaction mixture was quenched by water (10 mL). The resulting mixture was extracted with EA (5 mL×3). The combined organic layers were washed with brine, dried over anhydrous Na₂SO₄. After filtration, the filtrate was concentrated under reduced pressure. The crude product 30 mg was purified by Prep-HPLC with the following conditions (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 3-tert-butyl-1-{1-[1-(3-cyclobutylphenyl)ethyl]-2-oxoquinoxalin-6-yl}urea (7.8 mg, 14.70%) as a yellow solid. MS (ESI): mass calcd. C₂₅H₃₀N₄O₂ for: 418.24 m/z, found 419.15 [M+H]⁺. ¹H NMR (400 MHz, DMSO-d₆) δ 8.39 (s, 1H), 8.27 (s, 1H), 7.92 (d, J=2.6 Hz, 1H), 7.21-7.32 (m, 2H), 6.98-7.21 (m, 4H), 6.55-6.59 (m, 1H), 6.03 (s, 1H), 3.45-3.51 (m, 1H), 2.16-2.30 (m, 2H), 1.98-2.09 (m, 2H), 1.90-1.98 (m, 1H), 1.82-1.89 (m, 3H), 1.72-1.81 (m, 1H), 1.30 (s, 9H).

The following examples in Table 15 were made according to the procedure for example 246.

TABLE 15

| Structure | Ex. | IUPAC Name | 1H NMR | LCMS |
|---|---|---|---|---|
| 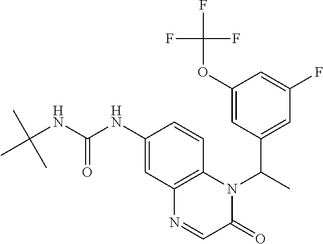 [SMILES: CC(c1cc(F)cc(OC(F)(F)F)c1)n1c(=O)cnc2cc(NC(=O)NC(C)(C)C)ccc21] | 247 | 1-(tert-butyl)-3- (1-(1-(3-fluoro-5- (tri- fluoromethoxy) phenyl)ethyl)-2- oxo-1,2- dihydro- quinoxalin- 6-yl)urea | ¹H NMR (300 MHZ, Methanol-d₄) δ 8.25 (s, 1H), 7.92 (d, J = 2.6 Hz, 1H), 7.41 - 7.50 (m, 1H), 7.10 - 7.19 (m, 2H), 7.07 (s, 2H), 6.66 (br, 1H), 1.98 (d, J = 7.1 Hz, 3H), 1.38 (s, 9H). | calcd. For C₂₂H₂₂F₄N₄O₃: 466.16 m/z, found: 467.05 [M + H]⁺ |
| 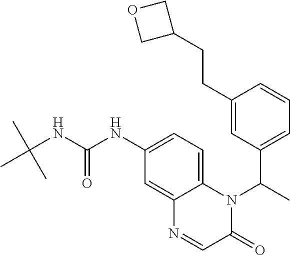 [SMILES: CC(c1cccc(CCC2COC2)c1)n1c(=O)cnc2cc(NC(=O)NC(C)(C)C)ccc21] | 248 | 1-(tert-butyl)-3- (1-(1-(3-(2- (oxetan-3- yl)ethyl)phenyl) ethyl)-2-oxo-1,2- dihydro- quinoxalin- 6-yl)urea | ¹H NMR (400 MHZ, Acetonitrile-d₃) δ 8.24 (s, 1H), 7.84 (d, J = 2.5 Hz, 1H), 7.24- 7.36 (m, 2H), 7.00- 7.18 (m, 5H), 6.69 (s, 1H), 5.10 (s, 1H), 4.49-4.60 (m, 2H), 4.15-4.23 (m, 2H), 2.75-2.91 (m, 1H), 2.45-2.56 (m, 2H), 2.13-2.17 (m, 1H), 1.83-1.98 (m, 4H), 1.27-1.36 (m, 9H). | calcd. for C₂₇H₃₅N₅O₃: 448.2 m/z, found 449.20 [M + H]⁺ |
| 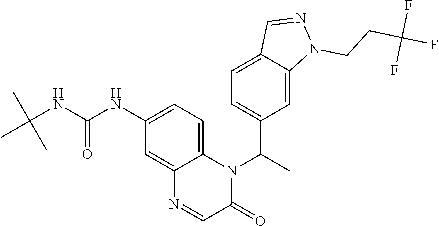 [SMILES: CC(c1ccc2cnn(CCC(F)(F)F)c2c1)n1c(=O)cnc2cc(NC(=O)NC(C)(C)C)ccc21] | 249 | 1-(tert-butyl)-3- (2-oxo-1-(1-(1- (3,3,3- trifluoropropyl)- 1H-indazol-6- yl)ethyl)-1,2- dihydro- quinoxalin- 6-yl)urea | ¹H NMR (300 MHZ, DMSO-d₆) δ 8.29- 8.35 (d, J = 18 Hz, 2H), 8.08 (s, 1H), 7.92 (s, 1H), 7.80 (s, 1H), 7.63-7.66 (d, J = 9 Hz, 1H), 7.15-7.16 (m, 2H), 6.88-6.91 (m, 1H), 6.76 (s, 1H), 5.99 (s, 1H), 4.69- 4.74 (t, J = 15 Hz, 2H), 2.87-2.96 (m, 2H), 1.96-1.99 (m, | calcd. for C₂₅H₂₇F₃N₆O₂: 500.21 m/z, found: 501.05 [M + H]⁺ |
| | | | 3H), 1.27 (s, 9H). | |
| | | | ¹⁹F NMR (282 MHZ, | |
| | | | DMSO) δ −63.77. | |
| 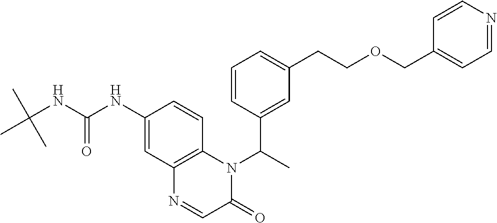 [SMILES: CC(c1cccc(CCOCc2ccncc2)c1)n1c(=O)cnc2cc(NC(=O)NC(C)(C)C)ccc21] | 250 | 1-(tert-butyl)-3- (2-oxo-1-(1-(3- (2-(pyridin-4- ylmethoxy)ethyl) phenyl)ethyl)- 1,2- dihydro- quinoxalin- 6-yl)urea | ¹H NMR (400 MHZ, DMSO-d₆) δ 8.52- 8.46 (m, 2H), 8.39 (s, 1H), 8.26 (s, 1H), 7.03-7.38 (m, 8H), 6.63-6.68 (m, 1H), 6.03 (s, 1H), 4.47 (s, 2H), 3.64 (t, J = 6.6 Hz, 2H), 2.85 (t, J = 6.6 Hz, 2H), 1.87 (d, J = 7.1 Hz, 3H), 1.28 (s, 9H). | calcd. for C₂₉H₃₃N₅O₃, 449.28 m/z, found 500.25 [M + H]⁺ |
| 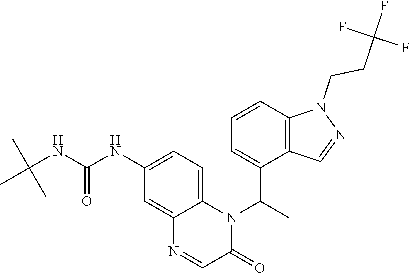 [SMILES: CC(c1cccc2c1cnn2CCC(F)(F)F)n1c(=O)cnc2cc(NC(=O)NC(C)(C)C)ccc21] | 251 | 1-(tert-butyl)-3- (2-oxo-1-(1-(1- (3,3,3- trifluoropropyl)- 1H-indazol-4- yl)ethyl)-1,2- dihydro- quinoxalin- 6-yl)urea | ¹H NMR (400 MHZ, DMSO-d₆) δ 8.38 (s, 1H), 8.32 (s, 1H), 7.82-7.92 (m, 1H), 7.65-7.76 (m, 1H), 7.46-7.51(m, 2H), 7.21-7.41 (m, 3H), 6.90 (s, 1H), 6.00 (s, 1H), 4.52- 4.70 (m, 2H), 2.80- 2.94 (m, 2H), 1.98- 2.08 (m, 3H), 1.25- 1.30 (m, 9H). ¹⁹F NMR (376 MHZ, DMSO) δ −63.94 | calcd. for C₂₅H₂₇F₃N₆O₂: 500.21, found: 501.05 [M + H]⁺ |
| 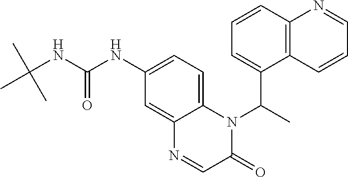 [SMILES: CC(c1cccc2ncccc12)n1c(=O)cnc2cc(NC(=O)NC(C)(C)C)ccc21] | 252 | 1-(tert-butyl)-3- (2-oxo-1-(1- (quinolin-5- yl)ethyl)-1,2- dihydro- quinoxalin- 6-yl)urea | ¹H NMR (400 MHZ, DMSO-d₆) δ 8.83 (dd, J = 4.0, 1.5 Hz, 1H), 8.39 (s, 1H), 8.23 (s, 1H), 8.04 (dd, J = 19.4, 7.9 Hz, 3H), 7.82-7.91 (m, 2H), 7.54 (s, 1H), 7.43 (dd, J = 8.7, 4.2 Hz, 1H), 7.34 (s, 1H), | calcd. for C₂₄H₂₅N₅O₂: 415.20 m/z, found 416.05 [M + H]⁺ |
| | | | 6.97 (s, 1H), 6.02 (s, | |
| | | | 1H), 2.04 (d, J = 7.0 | |
| | | | Hz, 3H), 1.27 (s, 9H). | |
| 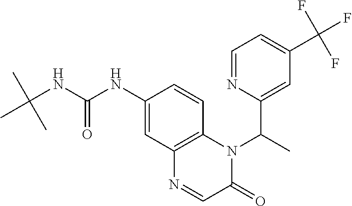 [SMILES: CC(c1cc(C(F)(F)F)ccn1)n1c(=O)cnc2cc(NC(=O)NC(C)(C)C)ccc21] | 253 | 1-(tert-butyl)-3- (2-oxo-1-(1-(4- (trifluoromethyl) pyridin-2- yl)ethyl)-1,2- dihydro- quinoxalin- 6-yl)urea | ¹H NMR (400 MHZ, DMSO-d₆) δ 8.78 (d, J = 5.1 Hz, 1H), 8.44 (s, 1H), 8.20 (s, 1H), 7.94 (d, J = 2.5 Hz, 1H), 7.74 (s, 1H), 7.69 (d, J = 5.2 Hz, 1H), 6.93 - 7.50 (m, 2H), 6.04 (s, 1H), 1.93 (d, J = 7.0 Hz, 3H), 1.30 (s, 9H). 19F NMR (376 MHZ, | calcd. for C₂₁H₂₂F₃N₅O₂: 433.17 m/z, found 434.00 [M + H]⁺ |
| | | | DMSO) δ(ppm): | |
| | | | −63.0934. | |
| 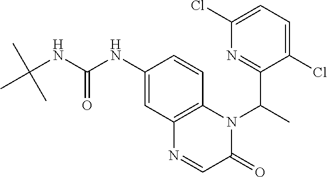 [SMILES: CC(c1nc(Cl)ccc1Cl)n1c(=O)cnc2cc(NC(=O)NC(C)(C)C)ccc21] | 254 | 1-(tert-butyl)-3- (1-(1-(3,6- dichloropyridin- 2-yl)ethyl)-2- oxo-1,2- dihydro- quinoxalin- 6-yl)urea | ¹H NMR (400 MHZ, DMSO-d₆) δ 8.45 (s, 1H), 8.15 (s, 1H), 7.85-8.04(m, 2H), 7.56(d, J = 8.3 Hz, 1H),7.26-7.50(m, 2H), 6.41(d, J =7.3 Hz, 1H),6.07(s, 1H), 1.80(d, J = 6.9 Hz, 3H), 1.30 (s, 9H) | calcd. for C₂₀H₂₁Cl₂N₅O₂: 433.11, found: 434.15 [M + H]⁺ |

### Example 255: 1-((R)-2-methyl-3-oxo-4-((S)-1-phenylethyl)-3,4-dihydro-2H-benzo[b][1,4]oxazin-7-yl)-3-(1-methylcyclobutyl)urea

### Synthetic Scheme

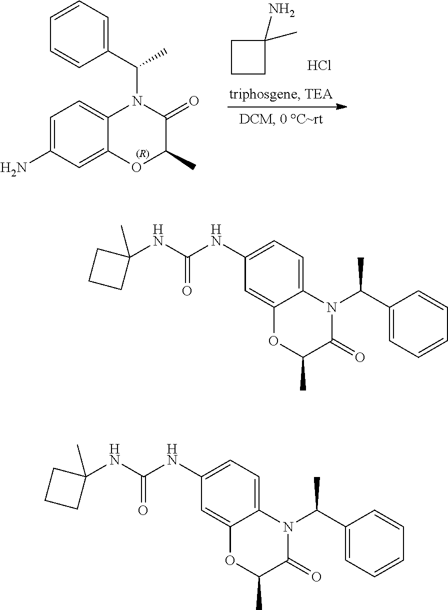

### 1-((R)-2-methyl-3-oxo-4-((S)-1-phenylethyl)-3,4-dihydro-2H-benzo[b][1,4]oxazin-7-yl)-3-(1-methylcyclobutyl)urea

To a stirred mixture of (R)-7-amino-2-methyl-4-((S)-1-phenylethyl)-2H-benzo[b][1,4]oxazin-3(4H)-one (100 mg, 0.354 mmol, 1 equiv) in DCM (5 mL) was added triphosgene (42.04 mg, 0.142 mmol, 0.4 equiv) at 0 degrees C. under nitrogen atmosphere. After 30 min, the TEA (107.52 mg, 1.062 mmol, 3 equiv) was added at 0 degrees C. After stirring for 30 min at 0 degrees C., the 1-methylcyclobutan-1-amine (60.32 mg, 0.708 mmol, 2 equiv) and TEA (107.52 mg, 1.062 mmol, 3 equiv) was added to the mixture at 0 degrees C., The resulting mixture was stirred for 2 h at room temperature. Desired product could be detected by LCMS. The mixture was diluted with water (20 mL). The resulting mixture was extracted with EA (3×20 mL). The organic layers were combined, dried over anhydrous Na₂SO₄, filtered and concentrated. The residue was purified by preparative HPLC using a X Bridge Prep OBD C8 150 mm×30 mm×5m column (eluent: 1700 to 42% (v/v) CH₃CN and H₂O with 10 mmol/L NH₄HCO₃) to afford the 1-((R)-2-methyl-3-oxo-4-((S)-1-phenylethyl)-3,4-dihydro-2H-benzo[b][1,4]oxazin-7-yl)-3-(1-methylcyclobutyl)urea (63.0 mg, 45.21%) as a white solid. MS (ESI): mass calcd. for C₂₃H₂₇N₃O₃: 393.21 m/z, found 394.05[M+H]⁺. ¹H—NVR (300 MHz, DMSO-d₆) δ 8.20 (s, 1H), 7.31-7.42 (m, 2H), 7.20-7.31 (m, 4H), 6.55-6.69 (m, 2H), 6.22 (s, 1H), 6.03-6.16 (i, 1H), 4.69-4.82 (m, 1H), 2.17-2.32 (m, 2H), 1.81-1.93 (i, 2H), 1.65-1.80 (m, 5H), 1.44 (d, J=6.8 Hz, 3H), 1.36 (s, 3H).

The following examples in Table 16 were made according to the procedure for example 255.

TABLE 16

| Structure | Ex. | IUPAC Name | ¹H NMR | LCMS |
|---|---|---|---|---|
| 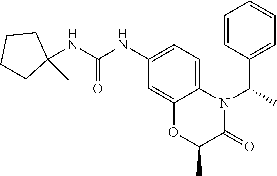 [SMILES: C[C@H]1Oc2cc(NC(=O)NC3(C)CCCC3)ccc2N([C@@H](C)c2ccccc2)C1=O] | 256 | 1-((R)-2-methyl-3- oxo-4-((S)-1-phenyl- ethyl)-3,4-dihydro- 2H-benzo[b][1,4] oxazin-7-yl)-3-(1- methylcyclopentyl)- urea | ¹H-NMR (300 MHz, DMSO-d₆) δ 8.16 (s, 1H), 7.31-7.42 (m, 2H), 7.20-7.31 (m, 4H), 6.60 (d, J = 2.2 Hz, 2H), 6.06-6.16 (m, 1H), 6.01 (s, 1H), 4.68-4.82 (m, 1H), 1.81-1.92 (m, 2H), 1.75 (d, J = 7.1 Hz, 3H), 1.49-1.68 (m, | calcd. for C₂₄H₂₉N₃O₃: 407.22 m/z, found: 408.10 [M + H]⁺ |
| | | | 6H), 1.40-1.47 (m, | |
| | | | 3H), 1.33 (s, 3H). | |
| 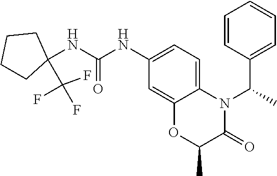 [SMILES: C[C@H]1Oc2cc(NC(=O)NC3(C(F)(F)F)CCCC3)ccc2N([C@@H](C)c2ccccc2)C1=O] | 257 | 1-((R)-2-methyl-3- oxo-4-((S)-1-phen- ylethyl)-3,4-dihydro- 2H-benzo[b][1,4] oxazin-7-yl)-3-(1- (trifluoromethyl)- cyclopentyl)urea | ¹H NMR (300 MHz, DMSO-d₆) δ 8.30 (s, 1H), 7.30-7.50 (m, 2H), 7.15-7.30 (m, 4H), 6.50-6.74 (m 2H), 6.47 (s, 1H), 5.98-6.20 (m, 1H), 4.60-4.87 (m, 1H), 2.13-2.25 (m, 2H), 1.82-1.97 (m, 2H), 1.63-1.80 (m, 7H), | calcd. for C₂₄H₂₆F₃N₃O₃: 461.19 m/z, found 462.15 [M + H]⁺ |
| | | | 1.44 (d, J = 6.8 Hz, | |
| | | | 3H). ¹⁹F NMR (282 | |
| | | | MHz, DMSO) δ | |
| | | | (ppm): −75.852. | |
| 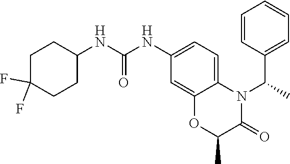 [SMILES: C[C@H]1Oc2cc(NC(=O)NC3CCC(F)(F)CC3)ccc2N([C@@H](C)c2ccccc2)C1=O] | 258 | 1-(4,4-difluoro- cyclohexyl)-3-((R)- 2-methyl-3-oxo-4- ((S)-1-phenyl- ethyl)-3,4-dihydro- 2H-benzo[b][1,4] oxazin-7-yl)urea | ¹H NMR (400 MHz, DMSO-d₆) δ 8.25 (s, 1H), 7.31-7.44 (m, 2H), 7.13-7.42 (m, 4H), 6.52-6.80 (m, 2H), 6.18-6.38 (m, 1H), 5.88-6.17(m, 1H), 4.55-4.94 (m, 1H), 3.65 (s, 1H), 1.90-2.05 (m, 4H), | calcd. for C₂₄H₂₇F₂N₃O₃: 443.20, found: 444.05 [M + H]⁺ |
| | | | 1.80-1.89 (m, 2H), | |
| | | | 1.70-1.79 (m, 3H), | |
| | | | 1.41-1.51(m, 5H). | |
| | | | ¹⁹F NMR (376 | |
| | | | MHz, DMSO) δ | |
| | | | −93.2970, 96.8730. | |
| 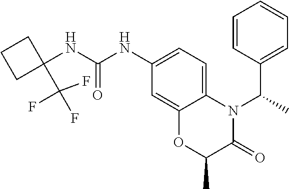 [SMILES: C[C@H]1Oc2cc(NC(=O)NC3(C(F)(F)F)CCC3)ccc2N([C@@H](C)c2ccccc2)C1=O] | 259 | 1-((R)-2-methyl- 3-oxo-4-((S)-1- phenylethyl)-3,4- dihydro-2H-benzo [b][1,4]oxazin-7- yl)-3-(1-(trifluoro- methyl)cyclo- butyl)urea | ¹H NMR (300 MHz, DMSO-d₆) δ 8.35 (s, 1H), 7.42-7.31 (m, 2H), 7.30-7.19 (m, 4H), 6.88 (s, 1H), 6.78-6.58 (m, 2H), 6.10 (q, J = 7.2 Hz, 1H), 4.76 (q, J = 6.6 Hz, 1H), 2.47-2.32 (m, 4H), 2.02-1.92 (m, 2H), 1.76 (d, J = 7.2 Hz, 3H), 1.44 (d, | calcd. for C₂₃H₂₄F₃N₃O₃: 447.2 m/z, found: 448.15 [M + H]⁺. |
| | | | J = 6.6 Hz, 3H). ¹⁹F | |
| | | | NMR (282 MHz, | |
| | | | DMSO-d₆) δ −79.87. | |
| 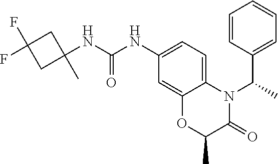 [SMILES: C[C@H]1Oc2cc(NC(=O)NC3(C)CC(F)(F)C3)ccc2N([C@@H](C)c2ccccc2)C1=O] | 260 | 1-(3,3-difluoro-1- methylcyclobutyl)- 3-((R)-2-methyl-3- oxo-4-((S)-1-phen- ylethyl)-3,4- dihydro-2H- benzo[b][1,4] oxazin-7-yl)urea | ¹H NMR (300 MHz, DMSO-d₆) δ 8.35 (s, 1H), 6.98-7.50 (m, 6H), 6.40-6.84 (m, 3H), 6.10 (q, J = 7.1 Hz, 1H), 4.76 (q, J = 6.8 Hz, 1H), 2.88 (q, J = 13.9 Hz, 2H), 2.51-2.65 (m, 2H), 1.76 (d, J = 7.1 Hz, | calcd. for C₂₃H₂₅F₂N₃O₃: 429.19 m/z, found 430.20 [M + H]⁺ |
| | | | 3H), 1.37-1.50 (m, | |
| | | | 6H). ¹⁹F NMR (282 | |
| | | | MHz, DMSO) δ | |
| | | | (ppm): −85.659, | |
| | | | −89.598. | |
| 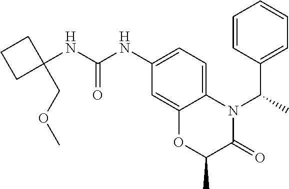 [SMILES: COCC1(NC(=O)Nc2ccc3c(c2)O[C@H](C)C(=O)N3[C@@H](C)c2ccccc2)CCC1] | 261 | 1-(1-(methoxy- methyl)cyclobutyl)- 3-((R)-2-methyl-3- oxo-4-((S)-1-phen- ylethyl)-3,4- dihydro-2H-benzo [b][1,4]oxazin-7- yl)urea | ¹H NMR (300 MHz, DMSO-d₆) δ 8.36 (s, 1H), 7.36-7.33 (m, 2H), 7.28-7.23 (m, 4H), 6.62-6.60 (m, 2H), 6.18 (s, 1H), 6.10 (q, J = 7.2 Hz, 1H), 4.75 (q, J = 6.9 Hz, 1H), 3.45 (s, 2H), 3.30 (s, 3H), 2.26- 2.22 (m, 2H), 2.01- 1.84 (m, 2H), 1.75 | calcd. for C₂₄H₂₉N₃O₄: 423.2 m/z, found: 424.15 [M + H]⁺. |
| | | | (d, J = 7.2 Hz, 3H), | |
| | | | 1.43 (d, J = 6.6 Hz, | |
| | | | 3H). | |
| 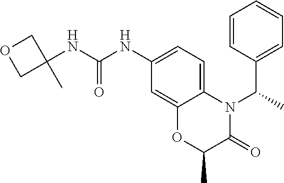 [SMILES: C[C@H]1Oc2cc(NC(=O)NC3(C)COC3)ccc2N([C@@H](C)c2ccccc2)C1=O] | 262 | 1-((R)-2-methyl-3- oxo-4-((S)-1- phenylethyl)-3,4- dihydro-2H-benzo [b][1,4]oxazin-7- yl)-3-(3-methyl- oxetan-3-yl)urea | ¹H NMR (400 MHz, DMSO-d₆) δ 8.46 (s, 1H), 7.19-7.42 (m, 6H), 6.52-6.78 (m, 3H), 6.01-6.18 (m, 1H), 4.70-4.88 (m, 1H), 4.50-4.65(m, 2H), 4.21-4.35 (m, 2H), 1.71-1.80 (m, 3H), 1.50 (s, 3H), 1.38-1.49 (m, 3H) | calcd. for C₂₂H₂₅N₃O₄: 395.18, found: 396.05 [M + H]⁺ |
| 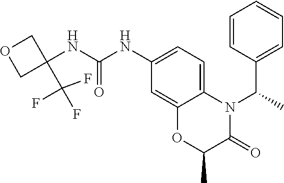 [SMILES: C[C@H]1Oc2cc(NC(=O)NC3(C(F)(F)F)COC3)ccc2N([C@@H](C)c2ccccc2)C1=O] | 263 | 1-((R)-2-methyl-3- oxo-4-((S)-1-phen- ylethyl)-3,4-dihydro- 2H-benzo[b][1,4] oxazin-7-yl)-3-(3- (trifluoromethyl)- oxetan-3-yl)urea | ¹H NMR (300 MHz, DMSO-d₆) δ 8.71 (s, 1H), 7.31-7.47 (m, 3H), 7.05-7.31 (m, 4H), 6.70-6.82 (m, 1H), 6.58-6.70 (m, 1H), 5.99-6.21 (m, 1H), 4.71-4.88 (m, 3H), 4.55-4.71 (m, 2H), 1.68-1.83 (m 3H), 1.39-1.51 (m, | calcd. for C₂₂H₂₂F₃N₃O₄, 449.16 m/z, found 450.10 [M + H]⁺ |
| | | | 3H). | |
| 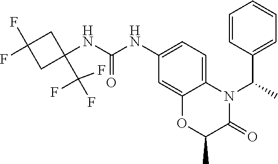 [SMILES: C[C@H]1Oc2cc(NC(=O)NC3(C(F)(F)F)CC(F)(F)C3)ccc2N([C@@H](C)c2ccccc2)C1=O] | 264 | 1-(3,3-difluoro-1- (trifluoromethyl)- cyclobutyl)-3-((R)- 2-methyl-3-oxo-4- ((S)-1-phenylethyl)- 3,4-dihydro-2H- benzo[b][1,4] oxazin-7-yl)urea | ¹H NMR (300 MHz, Methanol-d4) δ 7.24- 7.43 (m, 5H), 7.18 (d, J = 2.0 Hz, 1H), 6.64-6.77 (m, 2H), 6.25 (q, J = 7.2 Hz, 1H), 4.74 (q, J = 6.9 Hz, 1H), 3.13 (t, J = 11.6 Hz, 4H), 1.83 (d, J = 7.1 Hz, 3H), | calcd. for C₂₃H₂₂F₅N₃O₃: 483.16 m/z found: 484.15 [M + H]⁺. |
| | | | 1.54 (d, J = 6.8 Hz, | |
| | | | 3H). ¹⁹F-NMR (282 | |
| | | | MHz, MeOD) δ | |
| | | | −80.80, −92.37, | |
| | | | −93.07. | |
| 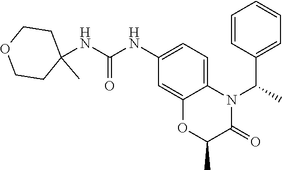 [SMILES: C[C@H]1Oc2cc(NC(=O)NC3(C)CCOCC3)ccc2N([C@@H](C)c2ccccc2)C1=O] | 265 | 1-((R)-2-methyl-3- oxo-4-((S)-1-phen- ylethyl)-3,4-dihydro- 2H-benzo[b][1,4] oxazin-7-yl)-3-(4- methyltetrahydro- 2H-pyran-4-yl)urea | ¹H NMR (400 MHz, DMSO-d₆) δ 8.26 (s, 1H), 7.32-7.49 (m, 2H), 7.18-7.31 (m, 4H), 6.50-6.68 (m, 2H), 6.08-6.18 (m, 1H), 5.90-6.07 (m, 1H), 4.64-4.88 (m, 1H), 3.48-3.71(m, 4H), 1.88-1.98 (m, 2H), 1.71-1.81 (m, | calcd. for C₂₄H₂₉N₃O₄: 423.22, found: 424.10 [M + H]⁺ |
| | | | 3H), 1.42-1.59 (m, | |
| | | | 5H), 1.28-1.38 (m, | |
| | | | 3H). | |

### Example 266: 3-tert-butyl-1-{1-[(2-cyano-5-methylphenyl)methyl]-2-oxo-3,4-dihydroquinolin-6-yl}urea

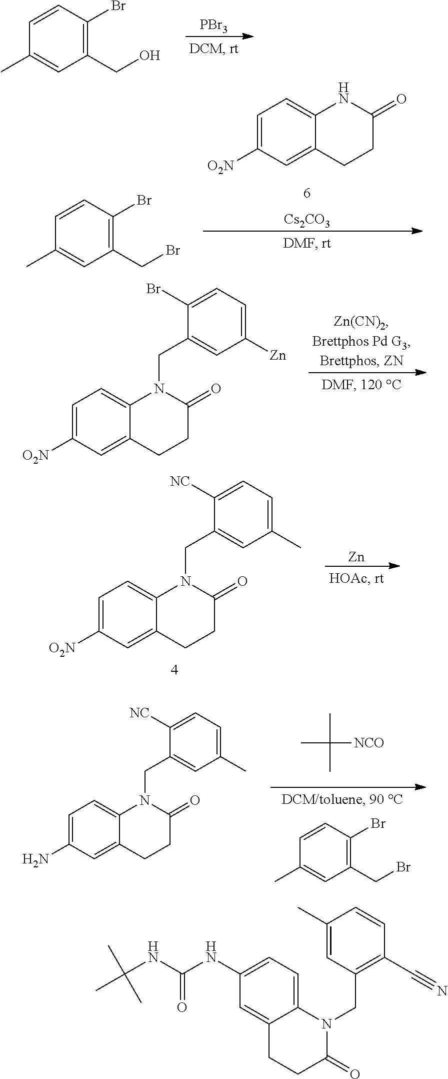

### 1-bromo-2-(bromomethyl)-4-methylbenzene

To a stirred solution of (2-bromo-5-methylphenyl) methanol (600 mg, 2.984 mmol, 1 equiv) in DCM (6 mL) was added mg, 8.952 mmol, 3 equiv) in portions at 0° C. under nitrogen atmosphere. The resulting mixture was stirred for 3 h at room temperature under nitrogen atmosphere. The reaction was monitored by LCMS. Quenched with water (30 mL) and extracted with EA (3×30 mL). The combined extracts were washed with water, brine, dried over Na₂SO₄, filtered and concentrated. The residue was purified by silica gel column chromatography, eluted with PE/EA (0-100%) to afford 1-bromo-2-(bromomethyl)-4-methylbenzene (300 mg, 38.09%) as a yellow solid. LC/MS (ESI): mass calcd. for C₈H₈Br₂: 263.90 m/z, found: 265.05, 267.05 [M+H, M+H+2]⁺.

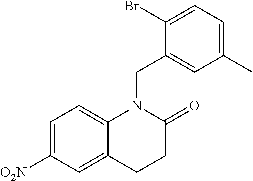 [SMILES: Cc1ccc(Br)c(CN2C(=O)CCc3cc([N+](=O)[O-])ccc32)c1]

### 1-[(2-bromo-5-methylphenyl)methyl]-6-nitro-3,4-dihydroquinolin-2-one

To a stirred solution of 1-bromo-2-(bromomethyl)-4-methylbenzene (280 mg, 1.061 mmol, 1 equiv) in DMF (3 mL) was added 6-nitro-3,4-dihydro-1H-quinolin-2-one (203.85 mg, 1.061 mmol, 1 equiv) and Cs₂CO₃ (691.24 mg, 2.122 mmol, 2 equiv) in portions at room temperature under nitrogen atmosphere. The resulting mixture was stirred for 3 h at room temperature under nitrogen atmosphere. The reaction was monitored by LCMS. Quenched with water (20 mL) and extracted with EA (3×30 mL). The combined extracts were washed with water, brine, dried over Na₂SO₄, filtered and concentrated. The residue was purified by silica gel column chromatography, eluted with PE/EA (0-100%) to afford 1-[(2-bromo-5-methylphenyl)methyl]-6-nitro-3,4-dihydroquinolin-2-one (100 mg, 25.12%) as a yellow solid. LC/MS (ESI): mass calcd. for C₁₇H₁₅BrN₂O₃: 374.03 m/z, found 375.00, 377.00 [M+H, M+H+2]⁺.

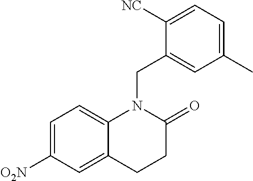 [SMILES: Cc1ccc(C#N)c(CN2C(=O)CCc3cc([N+](=O)[O-])ccc32)c1]

### 4-methyl-2-[(6-nitro-2-oxo-3,4-dihydroquinolin-1-yl)methyl]benzonitrile

To a stirred solution of 1-[(2-bromo-5-methylphenyl)methyl]-6-nitro-3,4-dihydroquinolin-2-one (150 mg, 0.400 mmol, 1 equiv) and Zn(CN)₂ (31.36 mg, 0.480 mmol, 1.2 equiv) in DMF (2 mL) was added BrettPhos Pd G3 (72.48 mg, 0.080 mmol, 0.2 equiv), BrettPhos (42.92 mg, 0.080 mmol, 0.2 equiv) and Zn (3.92 mg, 0.060 mmol, 0.15 equiv) in portions at room temperature under nitrogen atmosphere. The resulting mixture was stirred for 3 h at 120° C. under nitrogen atmosphere. The reaction was monitored by LCMS. The mixture was allowed to cool down to room temperature. Quenched with water (10 mL) and extracted with EA (3×10 mL). The combined extracts were washed with water, brine, dried over Na₂SO₄, filtered and concentrated. The residue was purified by silica gel column chromatography, eluted with PE/EA (0-100%) to afford 4-methyl-2-[(6-nitro-2-oxo-3,4-dihydroquinolin-1-yl)methyl]benzonitrile (50 mg, 38.92%) as a yellow solid. LC/MS (ESI): mass calcd. for C₁₈H₁₅N₃O₃:321.11 m/z, found 322.10 [M+H]⁺.

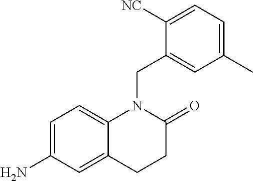 [SMILES: Cc1ccc(C#N)c(CN2C(=O)CCc3cc(N)ccc32)c1]

### 2-[(6-amino-2-oxo-3,4-dihydroquinolin-1-yl)methyl]-4-methylbenzonitrile

To a stirred solution of 4-methyl-2-[(6-nitro-2-oxo-3,4-dihydroquinolin-1-yl) methyl]benzonitrile (70 mg, 0.218 mmol, 1 equiv) in HOAc (1 mL) was added Zn (142.42 mg, 2.180 mmol, 10 equiv) in portions at room temperature under air atmosphere. The resulting mixture was stirred for 3 h at room temperature under air atmosphere. The reaction was monitored by LCMS. Quenched with water (30 mL) and extracted with EA (3×30 mL). The combined extracts were washed with water, brine, dried over Na₂SO₄, filtered and concentrated. This resulted in 2-[(6-amino-2-oxo-3,4-dihydroquinolin-1-yl)methyl]-4-methylbenzonitrile (60 mg, 94.53%) as a yellow solid. LC/MS (ESI): mass calcd. for C₁₈H₁₇N₃O:291.14 m/z, found 292.10 [M+H]⁺

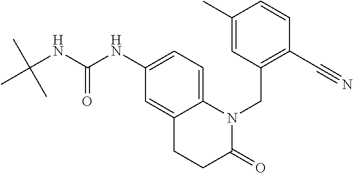 [SMILES: Cc1ccc(C#N)c(CN2C(=O)CCc3cc(NC(=O)NC(C)(C)C)ccc32)c1]

### 3-tert-butyl-1-{1-[(2-cyano-5-methylphenyl)methyl]-2-oxo-3,4-dihydroquinolin-6-yl}urea

To a stirred solution of 2-[(6-amino-2-oxo-3,4-dihydroquinolin-1-yl)methyl]-4-methylbenzonitrile (50 mg, 0.172 mmol, 1 equiv) in DCM/Toluene (2:1.1 mL) was added 2-isocyanato-2-methylpropane (51.04 mg, 0.516 mmol, 3 equiv) in portions at room temperature. The final reaction mixture was stirred overnight at 90° C. The reaction was monitored by LCMS. The mixture was allowed to cool down to room temperature. Quenched with water (10 mL) and extracted with EA (3×10 mL). The combined extracts were washed with water, brine, dried over Na₂SO₄, filtered and concentrated. The crude product was purified by Prep-HPLC using a X Bridge Prep OBD C18 150 mm×30 mm×5 μm column (eluent: 17% to 42% (v/v) CH₃CN and H₂O with 10 mmol/L NH₄HCO₃) to afford 3-tert-butyl-1-{1-[(2-cyano-5-methylphenyl)methyl]-2-oxo-3,4-dihydroquinolin-6-yl}urea (6.7 mg, 9.68%) as a white solid. LC/MS (ESI): mass calcd. for C₂₃H₂₆N₄O₂:390.20 m/z, found 391.15 [M+H]⁺. ¹H NMR (300 MHz, DMSO-d₆) δ 8.13 (s, 1H), 7.74 (d, J=8.1 Hz, 1H), 7.34 (s, 1H), 7.26 (d, J=8.1 Hz, 1H), 7.00 (d, J=11.4 Hz, 2H), 6.66 (d, J=9.0 Hz, 1H), 5.93 (s, 1H), 5.19 (s, 2H), 2.95-2.90 (m, 2H), 2.73-2.65 (m, 2H), 2.31 (s, 3H), 1.26 (s, 9H).

### Example 267: 1-(1-benzyl-5-chloro-2-oxo-1,2,3,4-tetrahydroquinolin-6-yl)-3-(tert-butyl)urea

### Synthetic Scheme

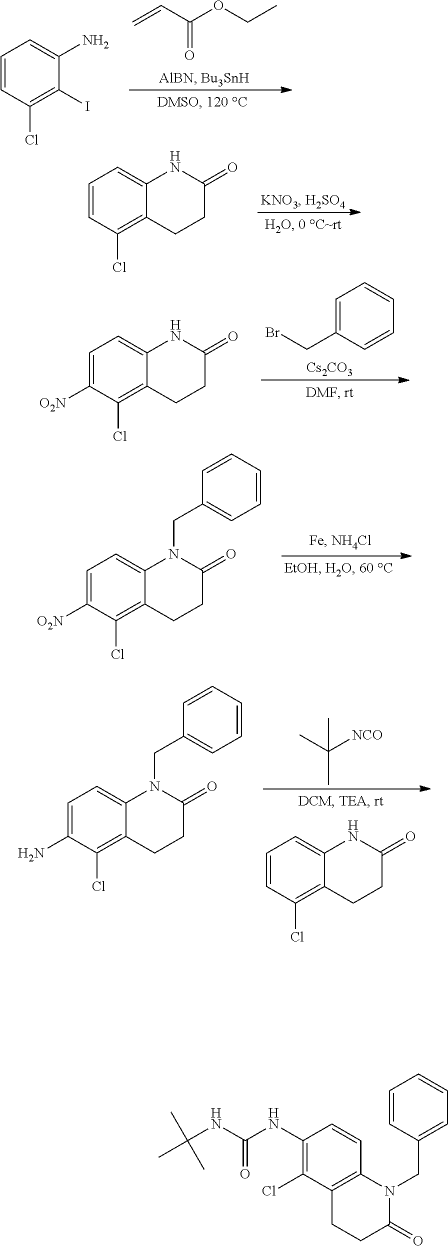

### 5-chloro-3,4-dihydroquinolin-2(1H)-one

To a solution of 3-chloro-2-iodoaniline (1.5 g, 5.918 mmol, 1 equiv), AIBN (388.71 mg, 2.367 mmol, 0.4 equiv) and tributyltin (2583.73 mg, 8.877 mmol, 1.5 equiv) in DMSO (10 mL) was added ethyl acrylate (2369.91 mg, 23.672 mmol, 4 equiv). The reaction was stirred at 120° C. under N₂ for 3 h. After cooling down to rt, the reaction was quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give 5-chloro-3,4-dihydro-1H-quinolin-2-one (348 mg, 32.38%) as a white solid. LC/MS: mass calcd. for C₉H₈ClNO: 181.03, found: 182.05 [M+H]⁺.

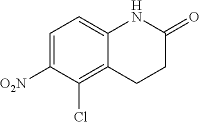 [SMILES: O=C1CCc2c(ccc([N+](=O)[O-])c2Cl)N1]

### 5-chloro-6-nitro-3,4-dihydroquinolin-2(1H)-one

To a solution of 5-chloro-3,4-dihydro-1H-quinolin-2-one (376 mg, 2.070 mmol, 1 equiv) in sulfuric acid (4 mL) was added KNO₃ (167.84 mg, 1.660 mmol, 0.80 equiv). The reaction was stirred at rt for 1 h. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give 5-chloro-6-nitro-3,4-dihydro-1H-quinolin-2-one (96 mg, 20.46%) as a white solid. LC/MS: mass calcd. for C₉H₇ClN₂O₃: 226.01, found: 226.85 [M+H]⁺.

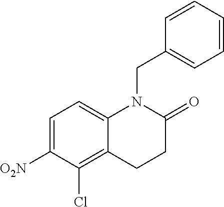 [SMILES: O=C1CCc2c(ccc([N+](=O)[O-])c2Cl)N1Cc1ccccc1]

### benzyl-5-chloro-6-nitro-3,4-dihydroquinolin-2(1H)-one

To a solution of 5-chloro-6-nitro-3,4-dihydro-1H-quinolin-2-one (151 mg, 0.666 mmol, 1 equiv) in dimethylformamide (3 mL) was added Cs₂CO₃ (653.31 mg, 1.998 mmol, 3 equiv) and (bromomethyl)benzene (653.31 mg, 1.998 mmol, 3 equiv). The reaction was stirred at rt for 1 h. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give 1-benzyl-5-chloro-6-nitro-3,4-dihydroquinolin-2-one (199 mg, 94.29%) as a white solid. LC/MS (ESI): mass calcd. for C₁₆H₁₃ClN₂O₃: 316.06 m/z, found:317.05 [M+H]f.

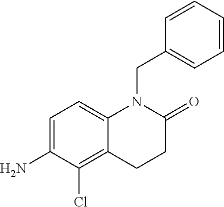 [SMILES: Nc1ccc2c(c1Cl)CCC(=O)N2Cc1ccccc1]

### 6-amino-1-benzyl-5-chloro-3,4-dihydroquinolin-2(1H)-one

To a solution of 1-benzyl-5-chloro-6-nitro-3,4-dihydroquinolin-2-one (100 mg, 0.316 mmol, 1 equiv) in ethyl alcohol (3 mL) was added iron (177 mg, 3.169 mmol, 10.04 equiv), NH₄Cl (167 mg, 3.122 mmol, 9.89 equiv) and water (0.3 mL). The reaction was stirred at 60° C. for 3 h. After cooling down to rt, the reaction was quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give 6-amino-1-benzyl-5-chloro-3,4-dihydroquinolin-2-one (74.5 mg, 82.29%) as a white solid. LC/MS (ESI): mass calcd. for C₁₆H₁₅ClN₂O: 286.09 m/z, found:287.05 [M+H]⁺.

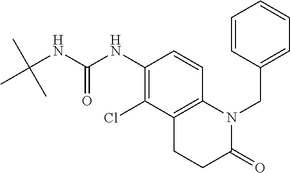 [SMILES: CC(C)(C)NC(=O)Nc1ccc2c(c1Cl)CCC(=O)N2Cc1ccccc1]

### 1-(1-benzyl-5-chloro-2-oxo-1,2,3,4-tetrahydroquinolin-6-yl)-3-(tert-butyl)urea

To a solution of 6-amino-1-benzyl-5-chloro-3,4-dihydroquinolin-2-one (70 mg, 0.244 mmol, 1 equiv) and 2-isocyanato-2-methylpropane (48.29 mg, 0.487 mmol, 2.00 equiv) in DCM (1 mL) was added TEA (73.93 mg, 0.731 mmol, 2.99 equiv). The reaction was stirred at rt for 3 h. Quenched with water (3 mL) and extracted with DCM (3×3 mL). The combined extracts were washed with water, brine, dried over Na₂SO₄, filtered and concentrated. The residue was purified by preparative HPLC using a XBridge Prep OBD C18 Column, 50 mm×30 mm×5 μm (eluent: 36% to 56% (v/v) CH₃CN and H₂O with 10 mmol/L NH₄HCO₃) to afford the title compound 1-(1-benzyl-5-chloro-2-oxo-3,4-dihydroquinolin-6-yl)-3-tert-butylurea (23.4 mg, 24.79%) as a white solid. LC/MS (ESI): mass calcd. for C₂₁H₂₄ClN₃O₂:385.16 m/z, found:386.00 [M+H]⁺. ¹H NMR (400 MHz, DMSO-d₆) δ 7.78-7.85 (m, 2H), 7.26-7.34 (m, 2H), 7.18-7.26 (m, 3H), 6.87-6.91 (m, 2H), 5.13 (s, 2H), 3.04-3.05 (m, 2H), 2.68-2.71 (m, 2H), 1.26 (s, 9H).

### Example 268 and Example 269: 1-(tert-butyl)-3-((S)-2-methyl-3-oxo-4-((S)-1-phenylethyl)-3,4-dihydro-2H-benzo[b][1,4]oxazin-7-yl)urea and 1-(tert-butyl)-3-((R)-2-methyl-3-oxo-4-((S)-1-phenylethyl)-3,4-dihydro-2H-benzo[b][1,4]oxazin-7-yl)urea

### Synthetic Scheme

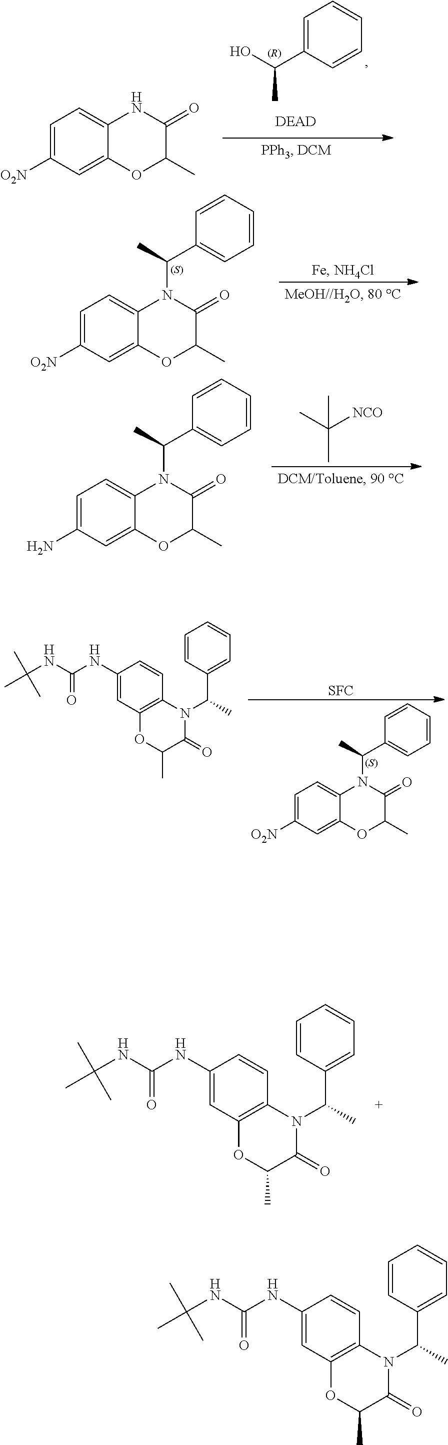

### 2-methyl-7-nitro-4-((S)-1-phenylethyl)-2H-benzo[b][1,4]oxazin-3(4H)-one

To a solution of 2-methyl-7-nitro-2,4-dihydro-1,4-benzoxazin-3-one (350 mg, 1.681 mmol, 1 equiv) in DCM (10 mL) was added (R)-1-phenylethan-1-ol (205.40 mg, 1.681 mmol, 1 equiv) and PPh₃ (661.48 mg, 2.522 mmol, 1.5 equiv). The reaction was stirred at rt under N₂ for 0.5 h. To the above mixture was added DEAD (439.21 mg, 2.522 mmol, 1.5 equiv) dropwise at 0° C. The resulting mixture was stirred for additional 1 hour at rt. Quenched with water (50 mL) and extracted with DCM (3×50 mL). The combined extracts were washed with water, brine, dried over Na₂SO₄, filtered and concentrated. The residue was purified by column chromatography on silica gel with EA/PE (0-100%) to give 2-methyl-7-nitro-4-[(1S)-1-phenylethyl]-2H-1,4-benzoxazin-3-one (270 mg, 51.42%) as a yellow solid. MS (ESI): mass calcd. for C₁₇H₁₆N₂O₄: 312.11 m/z, found 313.15 [M+H]⁺.

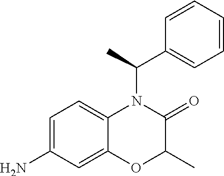 [SMILES: CC1Oc2cc(N)ccc2N([C@@H](C)c2ccccc2)C1=O]

### 7-amino-2-methyl-4-((S)-1-phenylethyl)-2H-benzo[b][1,4]oxazin-3(4H)-one

To a solution of 2-methyl-7-nitro-4-[(1S)-1-phenylethyl]-2H-1,4-benzoxazin-3-one (270 mg, 0.864 mmol, 1 equiv) in MeOH/H₂O (10:1, 11 mL) was added Fe (482.77 mg, 8.640 mmol, 10 equiv) and NH₄Cl (462.41 mg, 8.640 mmol, 10 equiv). The reaction was stirred at 80° C. for 1 hour. The resulting mixture was filtered, and the filter cake was washed with EA (3×30 mL). The filtrate was concentrated under reduced pressure. The mixture was diluted with water (30 mL) and extracted with EA (3×30 mL). The combined extracts were washed with water, brine, dried over Na₂SO₄, filtered and concentrated. This resulted in 7-amino-2-methyl-4-[(1S)-1-phenylethyl]-2H-1,4-benzoxazin-3-one (200 mg, 81.96%) as a yellow oil. MS (ESI): mass calcd. for C₁₇H₁₈N₂O₂: 282.14 m/z, found 283.00 [M+H]⁺.

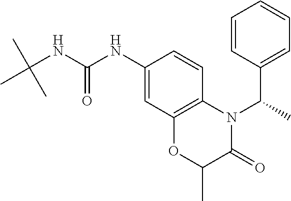 [SMILES: CC1Oc2cc(NC(=O)NC(C)(C)C)ccc2N([C@@H](C)c2ccccc2)C1=O]

### 1-(tert-butyl)-3-(2-methyl-3-oxo-4-((S)-1-phenylethyl)-3,4-dihydro-2H-benzo[b][1,4]oxazin-7-yl)urea

To a solution of 7-amino-2-methyl-4-[(1S)-1-phenylethyl]-2H-1,4-benzoxazin-3-one (130 mg, 0.460 mmol, 1 equiv) in DCM/Toluene (2:1, 15 mL) was added 2-isocyanato-2-methylpropane (273.86 mg, 2.760 mmol, 6 equiv). The reaction was stirred at 90° C. for 2 hours. After cooling to rt, the reaction was quenched with water (50 mL) and extracted with EA (3×50 mL). The combined extracts were washed with water, brine, dried over Na₂SO₄, filtered and concentrated. The residue was purified by preparative HPLC using a XBridge Shield RP18 OBD Column, 30*150 mm, 5 μm column (eluent: 43% to 65% (v/v) CH₃CN and H₂O with 10 mmol/L NH₄HCO₃) to afford the title compound 3-tert-butyl-1-{2-methyl-3-oxo-4-[(1S)-1-phenylethyl]-2H-1,4-benzoxazin-7-yl}urea (40 mg, 22.77%) as a white solid. MS (ESI): mass calcd. for C₂₂H₂₇N₃O₃: 381.21 m/z, found:382.20 [M+H]⁺.

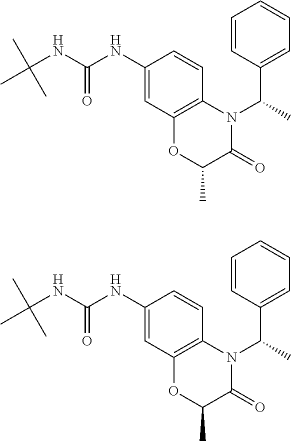

### 1-(tert-butyl)-3-((S)-2-methyl-3-oxo-4-((S)-1-phenylethyl)-3,4-dihydro-2H-benzo[b][1,4]oxazin-7-yl)urea and 1-(tert-butyl)-3-((R)-2-methyl-3-oxo-4-((S)-1-phenylethyl)-3,4-dihydro-2H-benzo[b][1,4]oxazin-7-yl)urea

A sample of 3-tert-butyl-1-{2-methyl-3-oxo-4-[(1S)-1-phenylethyl]-2H-1,4-benzoxazin-7-yl}urea (40 mg) was separated by chiral-HPLC using a CHIRALPAK IE, 2*25 cm, 5 μm column (eluent: 10% to 10% (v/v) Hex(0.5% 2M NH₃-MeOH) and EtOH) to yield first enantiomer example 3-tert-butyl-1-[(2S)-2-methyl-3-oxo-4-[(1S)-1-phenylethyl]-2H-1,4-benzoxazin-7-yl]urea (17.6 mg, 44.00%) as a white solid. MS(ESI): mass calcd. for C₂₂H₂₇N₃O₃: 381.21 m/z, found: 382.20 [M+H]⁺. ¹H NMR (400 MHz, DMSO-d₆) δ 8.11-8.29 (m, 1H), 7.25-7.45 (m, 6H), 6.55-6.68 (m, 1H), 6.35-6.55 (m, 1H), 6.11-6.26 (m, 1H), 5.85-6.03 (m, 1H), 4.63-4.79 (m, 1H), 1.64-1.71 (m, 3H), 1.43-1.59 (m, 3H), 1.25-1.26 (m, 9H) and to yield second enantiomer example 3-tert-butyl-1-[(2R)-2-methyl-3-oxo-4-[(1S)-1-phenylethyl]-2H-1,4-benzoxazin-7-yl]urea (17 mg, 42.50%) as a white solid. MS(ESI): mass calcd. for C₂₂H₂₇N₃O₃: 381.21 m/z, found: 382.20 [M+H]⁺. ¹H NMR (400 MHz, DMSO-d₆) δ 7.84-8.20 (m, 1H), 7.32-7.46 (m, 2H), 7.10-7.32 (m, 4H), 6.49-6.65 (m, 2H), 6.02-6.18 (m, 1H), 5.76-5.97 (m, 1H), 4.63-4.79 (m, 1H), 1.73-1.81 (m, 3H), 1.40-1.55 (m, 3H), 1.26-1.29 (m, 9H).

### Example 270: 1-(1-benzyl-7-cyano-2-oxo-3,4-dihydroquinolin-6-yl)-3-tert-butylurea

### Synthetic Scheme

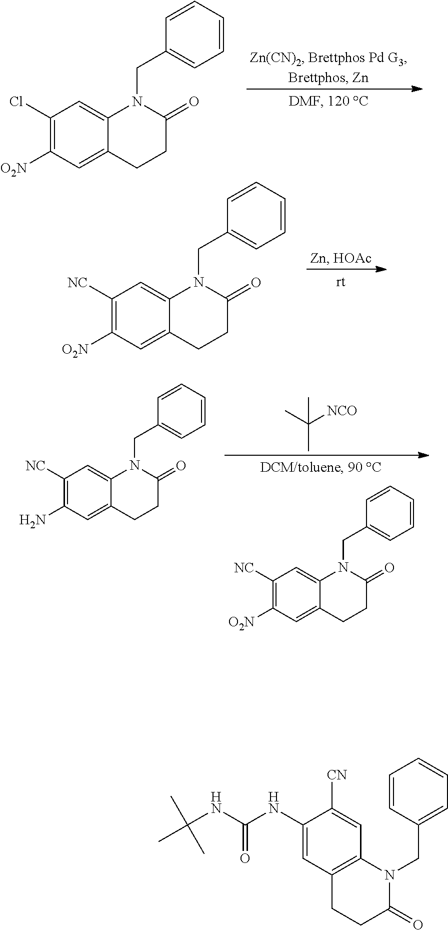

### 1-benzyl-6-nitro-2-oxo-3,4-dihydroquinoline-7-carbonitrile

To a stirred solution of 1-benzyl-7-chloro-6-nitro-3,4-dihydroquinolin-2-one (500 mg, 1.579 mmol, 1 equiv) in DMF (5 mL) was added Zn(CN)₂ (222.43 mg, 1.895 mmol, 1.2 equiv), BrettPhos Pd G3 (286.20 mg, 0.316 mmol, 0.2 equiv), BrettPhos (169.47 mg, 0.316 mmol, 0.2 equiv) and Zn (15.48 mg, 0.237 mmol, 0.15 equiv) in portions at room temperature under nitrogen atmosphere. The resulting mixture was stirred for 3 h at 120° C. under nitrogen atmosphere. The reaction was monitored by LCMS. The mixture was allowed to cool down to room temperature. Quenched with water (30 mL) and extracted with EA (3×30 mL). The combined extracts were washed with water, brine, dried over Na₂SO₄, filtered and concentrated. The residue was purified by silica gel column chromatography, eluted with PE/EA (0-100%) to afford 1-benzyl-6-nitro-2-oxo-3,4-dihydroquinoline-7-carbonitrile (200 mg, 41.23%) as a white solid. LC/MS (ESI): mass calcd. for C₁₇H₁₃N₃O₃: 307.10 m/z, found 308.05 [M+H]⁺.

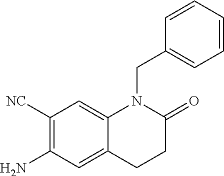 [SMILES: N#Cc1cc2c(cc1N)CCC(=O)N2Cc1ccccc1]

### 6-amino-1-benzyl-2-oxo-3,4-dihydroquinoline-7-carbonitrile

To a stirred solution of 1-benzyl-6-nitro-2-oxo-3,4-dihydroquinoline-7-carbonitrile (200 mg, 0.651 mmol, 1 equiv) in AcOH (2 mL) was added Zn (425.50 mg, 6.510 mmol, 10 equiv) in portions at room temperature under air atmosphere. The resulting mixture was stirred for 3 h at room temperature under air atmosphere. The reaction was monitored by LCMS. Quenched with water (10 mL) and extracted with EA (3×10 mL). The combined extracts were washed with water, brine, dried over Na₂SO₄, filtered and concentrated. The residue was purified by silica gel column chromatography, eluted with PE/EA (0-100%) to afford 6-amino-1-benzyl-2-oxo-3,4-dihydroquinoline-7-carbonitrile (80 mg, 44.44%) as a yellow solid. LC/MS (ESI): mass calcd. for C₁₇H₁₅N₃O: 277.12 m/z, found 278.05 [M+H]⁺.

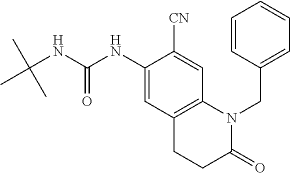 [SMILES: CC(C)(C)NC(=O)Nc1cc2c(cc1C#N)N(Cc1ccccc1)C(=O)CC2]

### 1-(1-benzyl-7-cyano-2-oxo-3,4-dihydroquinolin-6-yl)-3-tert-butylurea

To a stirred solution of 6-amino-1-benzyl-2-oxo-3,4-dihydroquinoline-7-carbonitrile (70 mg, 0.252 mmol, 1 equiv) in DCM/Toluene (2:1.1 mL) was added 2-isocyanato-2-methylpropane (75.07 mg, 0.756 mmol, 3 equiv) in portions at room temperature under nitrogen atmosphere. The resulting mixture was stirred overnight at 90° C. under air atmosphere. The reaction was monitored by LCMS. The mixture was allowed to cool down to room temperature. Quenched with water (10 mL) and extracted with DCM (3×10 mL). The combined extracts were washed with water, brine, dried over Na₂SO₄, filtered and concentrated. The crude product was purified by Prep-HPLC using a X Bridge Prep OBD C18 150 mm×30 mm×5 μm column (eluent: 26% to 45% (v/v) CH₃CN and H₂O with 10 mmol/L NH₄HCO₃) to afford 1-(1-benzyl-7-cyano-2-oxo-3,4-dihydroquinolin-6-yl)-3-tert-butylurea (3.9 mg, 4.01%) as a white solid. LC/MS (ESI): mass calcd. for C₂₂H₂₄N₄O₂: 376.19 m/z, found 377.15 [M+H]⁺. ¹H NMR (300 MHz, Methanol-d4) δ 7.82 (s, 1H), 7.35-7.31 (m, 2H), 7.24 (t, J=7.1 Hz, 3H), 7.11 (s, 1H), 5.20 (s, 2H), 3.08-3.01 (m, 2H), 2.80-2.75 (m, 2H), 1.35 (s, 9H).

### Example 271: 1-(tert-butyl)-3-(3-methyl-2-oxo-1-(1-(3-(trifluoromethyl)phenyl)ethyl)-1,2,3,4-tetrahydroquinazolin-6-yl)urea

### Synthetic Scheme

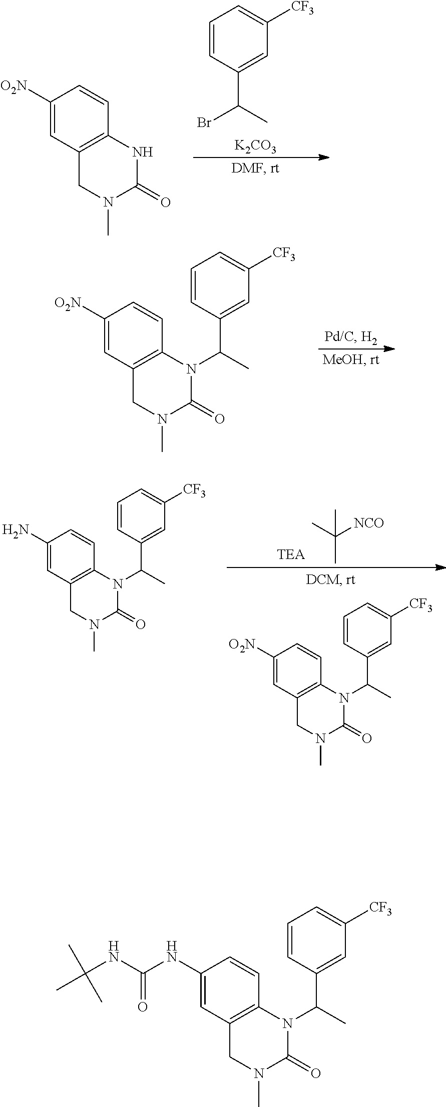

### 3-methyl-6-nitro-1-(1-(3-(trifluoromethyl)phenyl)ethyl)-3,4-dihydroquinazolin-2(1H)-one

To a solution of 3-methyl-6-nitro-1,4-dihydroquinazolin-2-one (200 mg, 0.966 mmol, 1 equiv) in DMF (5 mL) was added K₂CO₃ (0.40 g, 2.890 mmol, 3 equiv) and 1-(1-bromoethyl)-3-(trifluoromethyl)benzene (269 mg, 1.06 mmol, 1.1 equiv). The resulting mixture was placed at rt and stirred overnight. The reaction mixture was quenched by water (10 mL) and extracted with EA (3×10 mL). The combined organic extracts were washed with brine (10 mL), dried over anhydrous Na₂SO₄ and concentrated under vacuum. The crude product was purified by silica gel column to afford 3-methyl-6-nitro-1-(1-(3-(trifluoromethyl)phenyl)ethyl)-3,4-dihydroquinazolin-2(1H)-one (100 mg, 27.31%) as a yellow solid.

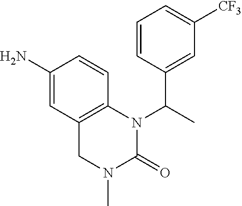 [SMILES: CC(c1cccc(C(F)(F)F)c1)N1C(=O)N(C)Cc2cc(N)ccc21]

### 6-amino-3-methyl-1-(1-(3-(trifluoromethyl)phenyl)ethyl)-3,4-dihydroquinazolin-2(1H)-one

To a solution of 3-methyl-6-nitro-1-(1-(3-(trifluoromethyl)phenyl)ethyl)-3,4-dihydroquinazolin-2(1H)-one (100 mg, 0.264 mmol, 1 equiv) in MeOH (10 mL) was added Pd/C (28.0 mg, 10%, 0.026 mmol, 0.1 equiv). The resulting mixture was placed at rt under a hydrogen atmosphere until the starting material was totally consumed by LCMS. The mixture was filtered and concentrated under vacuum to afford 6-amino-3-methyl-1-(1-(3-(trifluoromethyl)phenyl)ethyl)-3,4-dihydroquinazolin-2(1H)-one (60 mg, 65.15%) as a yellow solid. MS (ESI): mass calcd. for C₁₈H₁₈F₃N₃O, 349.14 m/z, found 350.10 [M+H]⁺.

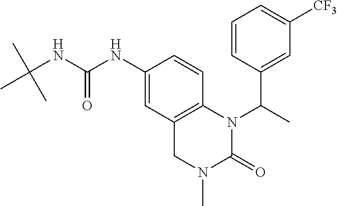 [SMILES: CC(c1cccc(C(F)(F)F)c1)N1C(=O)N(C)Cc2cc(NC(=O)NC(C)(C)C)ccc21]

### 1-(tert-butyl)-3-(3-methyl-2-oxo-1-(1-(3-(trifluoromethyl)phenyl)ethyl)-1,2,3,4-tetrahydroquinazolin-6-yl)urea

To a stirred solution of 6-amino-3-methyl-1-(1-(3-(trifluoromethyl)phenyl)ethyl)-3,4-dihydroquinazolin-2(1H)-one (100 mg, 0.286 mmol, 1 equiv) in DCM (10 mL) was added 2-isocyanato-2-methylpropane (142 mg, 1.431 mmol, 5 equiv) and TEA (87 mg, 0.859 mmol, 3 equiv) stirred overnight at rt. The reaction mixture was quenched by water (10 mL) and extracted with EA (3×20 mL). The combined organic extracts were washed with brine (10 mL), dried over anhydrous Na₂SO₄, and concentrated under vacuum to yield a crude product which was directly purified by flash chromatography (0-100% ethyl acetate/petroleum ether) to afford 100 mg crude product as a white solid. The compound was separated by preparative HPLC (Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 45% B to 73% B in 9 min, 73% B; Wave Length: 254/220 nm; RT1(min): 9.6; Number Of Runs: 0) to afford 1-(tert-butyl)-3-(3-methyl-2-oxo-1-(1-(3-(trifluoromethyl)phenyl)ethyl)-1,2,3,4-tetrahydroquinazolin-6-yl)urea (12.6 mg, 9.78%) as a white solid. MS (ESI): mass calcd. for C₂₃H₂₇F₃N₄O₂, 448.21 m/z, found 449.05 [M+H]⁺. ¹H NMR (300 MHz, DMSO-d₆) δ 8.09 (s, 1H), 7.58 (s, 4H), 7.30 (s, 1H), 6.88 (d, J=8.7 Hz, 1H), 6.40 (d, J=8.8 Hz, 1H), 5.83-5.96 (m, 2H), 4.32 (s, 2H), 2.91 (s, 3H), 1.78 (d, J=7.0 Hz, 3H), 1.26 (s, 9H).

### Example 272: 1-(1-benzyl-3-methyl-2-oxo-1,2-dihydroquinoxalin-6-yl)-3-(tert-butyl)urea

### Synthetic Scheme

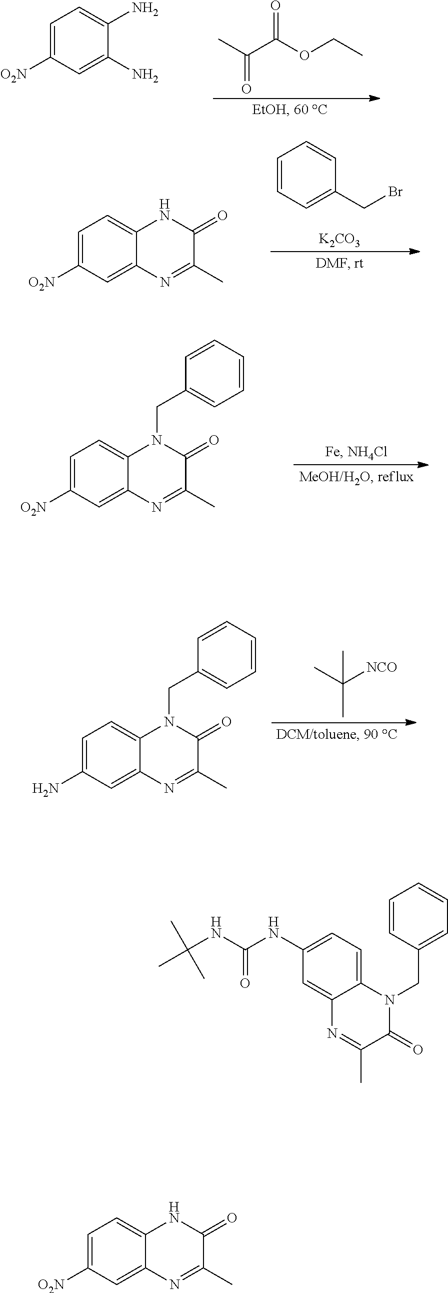

### 3-methyl-6-nitroquinoxalin-2(1H)-one

To a solution of 2-amino-4-nitroaniline (2 g, 13.060 mmol, 1 equiv) in ethyl alcohol (20 mL) was added ethyl pyruvate (1.82 g, 15.674 mmol, 1.20 equiv). The reaction was stirred at rt for 3 h. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give 3-methyl-6-nitro-1H-quinoxalin-2-one (1.78 g, 66.43%) as a yellow solid. MS (ESI): mass calcd. for C₉H₇N₃O₃: 205.05m/z, found 206.05 [M+H]⁺

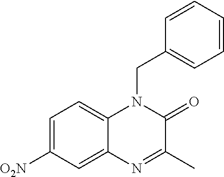 [SMILES: Cc1nc2cc([N+](=O)[O-])ccc2n(Cc2ccccc2)c1=O]

### benzyl-3-methyl-6-nitroquinoxalin-2(1H)-one

To a solution of 3-methyl-6-nitro-1H-quinoxalin-2-one (400 mg, 1.950 mmol, 1 equiv) and benzyl bromide (500.17 mg, 2.925 mmol, 1.5 equiv) in dimethylformamide (5 mL) was added K₂CO₃ (814.22 mg, 5.850 mmol, 3 equiv). The reaction was stirred at rt for 3 h. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give 1-benzyl-3-methyl-6-nitroquinoxalin-2-one (212 mg, 36.82%) as a white solid. MS (ESI): mass calcd. for C₁₆H₁₃N₃O₃: 295.10 m/z, found 296.10 [M+H]⁺

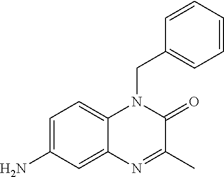 [SMILES: Cc1nc2cc(N)ccc2n(Cc2ccccc2)c1=O]

### 6-amino-1-benzyl-3-methylquinoxalin-2(1H)-one

To a solution of 1-benzyl-3-methyl-6-nitroquinoxalin-2-one (129 mg, 0.437 mmol, 1 equiv) in water (0.2 mL) and methanol (2 mL) was added iron (243.96 mg, 4.370 mmol, 10 equiv) and NH₄Cl (233.67 mg, 4.370 mmol, 10 equiv). The reaction was stirred at 60° C. for 3 h. After cooling down to rt, the reaction was quenched with water (5 mL) and extracted with EA (3×5 mL). The combined extracts were washed with water, brine, dried over Na₂SO₄, filtered and concentrated. The residue was purified by column chromatography on silica gel with EA/PE (0-100%) to give 6-amino-1-benzyl-3-methylquinoxalin-2-one (54.6 mg, 47.11%) as a white solid. LC/MS (ESI): mass calcd. for C₁₆H₁₅N₃O: 265.12 m/z, found:266.15 [M+H]⁺.

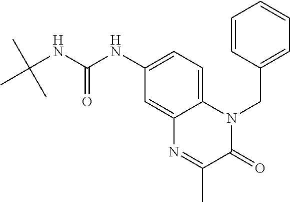 [SMILES: Cc1nc2cc(NC(=O)NC(C)(C)C)ccc2n(Cc2ccccc2)c1=O]

### 1-(1-benzyl-3-methyl-2-oxo-1,2-dihydroquinoxalin-6-yl)-3-(tert-butyl)urea

To a solution of 6-amino-1-benzyl-3-methylquinoxalin-2-one (55 mg, 0.207 mmol, 1 equiv) in DCM (1 mL)/toluene (0.5 mL) was added 2-isocyanato-2-methylpropane (61.65 mg, 0.621 mmol, 3 equiv). The reaction was stirred at 90° C. for 3 h. After cooling down to rt, the reaction was quenched with water (5 mL) and extracted with DCM (3×5 mL). The combined extracts were washed with water, brine, dried over Na₂SO₄, filtered and concentrated. The residue was purified by preparative HPLC using a XBridge Prep OBD C18 Column (eluent: 26% to 42% (v/v) CH₃CN and H₂O with 10 mmol/L NH₄HCO₃) to afford the title compound 1-(1-benzyl-3-methyl-2-oxoquinoxalin-6-yl)-3-tert-butylurea (15.3 mg, 20.22%) as a yellow solid. LC/MS (ESI): mass calcd. for C₂₁H₂₄N₄O₂:364.19 m/z, found:365.05 ¹H NMR (400 MHz, DMSO-d₆) δ 8.38 (s, 1H), 7.84 (s, 1H), 7.23-7.40 (m, 7H), 6.01 (s, 1H), 5.45 (s, 2H), 2.49 (s, 3H), 1.29 (s, 9H).

### Example 273: 3-tert-butyl-1-{1-[(3-chlorophenyl)methyl]-3-methyl-2-oxoquinoxalin-6-yl}urea

### Synthetic Scheme

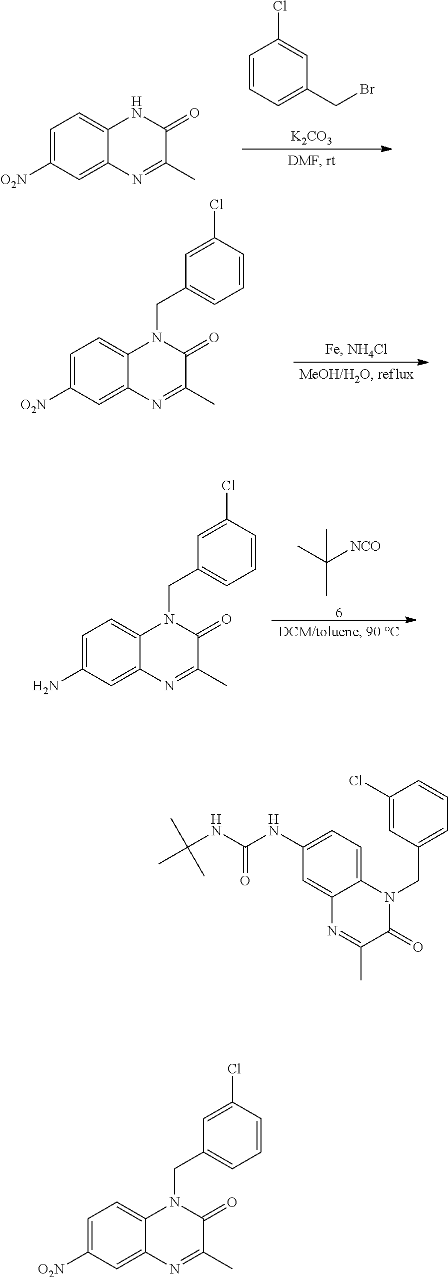

### 1-[(3-chlorophenyl) methyl]-3-methyl-6-nitroquinoxalin-2-one

To a stirred solution of 3-methyl-6-nitro-1H-quinoxalin-2-one (400 mg, 1.950 mmol, 1 equiv) and 1-(bromomethyl)-3-chlorobenzene (600.90 mg, 2.925 mmol, 1.5 equiv) in DMF (5 mL) was added K₂CO₃ (542.81 mg, 3.900 mmol, 2 equiv) in portions at room temperature under nitrogen atmosphere. The resulting mixture was stirred for 3 h at room temperature under nitrogen atmosphere. Quenched with water (30 mL) and extracted with EA (3×30 mL). The combined extracts were washed with water, brine, dried over Na₂SO₄, filtered and concentrated. The residue was purified by silica gel column chromatography, eluted with PE/EA (0-100%) to afford 1-[(3-chlorophenyl) methyl]-3-methyl-6-nitroquinoxalin-2-one (300 mg, 46.67%) as a yellow solid. LC/MS (ESI): mass calcd. for C₁₆H₁₂ClN₃O₃: 329.06 m/z, found 330.00 [M+H]⁺.

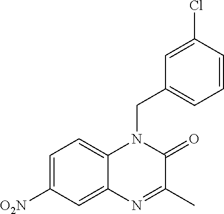 [SMILES: Cc1nc2cc([N+](=O)[O-])ccc2n(Cc2cccc(Cl)c2)c1=O]

### 6-amino-1-[(3-chlorophenyl) methyl]-3-methylquinoxalin-2-one

To a stirred solution of 1-[(3-chlorophenyl) methyl]-3-methyl-6-nitroquinoxalin-2-one (300 mg, 0.910 mmol, 1 equiv) in MeOH/H₂O (10:1, 4 mL) were added NH₄Cl (486.66 mg, 9.100 mmol, 10 equiv) and Fe (508.08 mg, 9.100 mmol, 10 equiv) at room temperature under nitrogen atmosphere. The resulting mixture was stirred for 3 h at 60° C. under nitrogen atmosphere. The reaction was monitored by LCMS. The mixture was allowed to cool down to room temperature. Quenched with water (20 mL) and extracted with EA (3×20 mL). The combined extracts were washed with water, brine, dried over Na₂SO₄, filtered and concentrated. The residue was purified by silica gel column chromatography, eluted with CH₂Cl₂/MeOH (10:1) to afford 6-amino-1-[(3-chlorophenyl) methyl]-3-methylquinoxalin-2-one (240 mg, 88.00%) as a white solid. LC/MS (ESI): mass calcd. for C₁₆H₁₄ClN₃O: 299.08 m/z, found 300.00 [M+H]⁺.

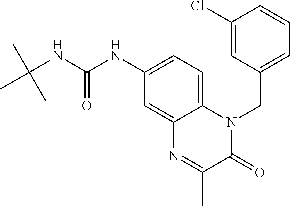 [SMILES: Cc1nc2cc(NC(=O)NC(C)(C)C)ccc2n(Cc2cccc(Cl)c2)c1=O]

### 3-tert-butyl-1-{1-[(3-chlorophenyl) methyl]-3-methyl-2-oxoquinoxalin-6-yl}urea

To a stirred solution of 6-amino-1-[(3-chlorophenyl) methyl]-3-methylquinoxalin-2-one (100 mg, 0.334 mmol, 1 equiv) in DCM/Toluene (2:1.1 mL) was added 2-isocyanato-2-methylpropane (99.21 mg, 1.002 mmol, 3 equiv) in portions at room temperature under nitrogen atmosphere. The resulting mixture was stirred overnight at 90° C. under nitrogen atmosphere. The reaction was monitored by LCMS. Quenched with water (10 mL) and extracted with DCM (3×10 mL). The combined extracts were washed with water, brine, dried over Na₂SO₄, filtered and concentrated. The crude product was purified by Prep-HPLC using a Xselect CSH OBD C18 150 mm×30 mm×5 μm column (eluent: 37% to 62% (v/v) CH₃CN and H₂O with 0.1% FA) to afford 3-tert-butyl-1-{1-[(3-chlorophenyl) methyl]-3-methyl-2-oxoquinoxalin-6-yl}urea (22.4 mg, 16.17%) as a yellow solid. LC/MS (ESI): mass calcd. for C₂₁H₂₃ClN₄O₂: 398.2 m/z, found 399.10 [M+H]⁺. ¹H NMR (300 MHz, DMSO-d₆) δ 8.40 (s, 1H), 7.84 (d, J=2.1 Hz, 1H), 7.38-7.28 (m, 6H), 6.02 (s, 1H), 5.45 (s, 2H), 2.49 (s, 3H) 1.30 (s, 9H).

### Example 274: 1-(tert-butyl)-3-(6-oxo-5-(3-(trifluoromethyl)benzyl)-5,6,7,8-tetrahydro-1,5-naphthyridin-2-yl)urea

### Synthetic Scheme

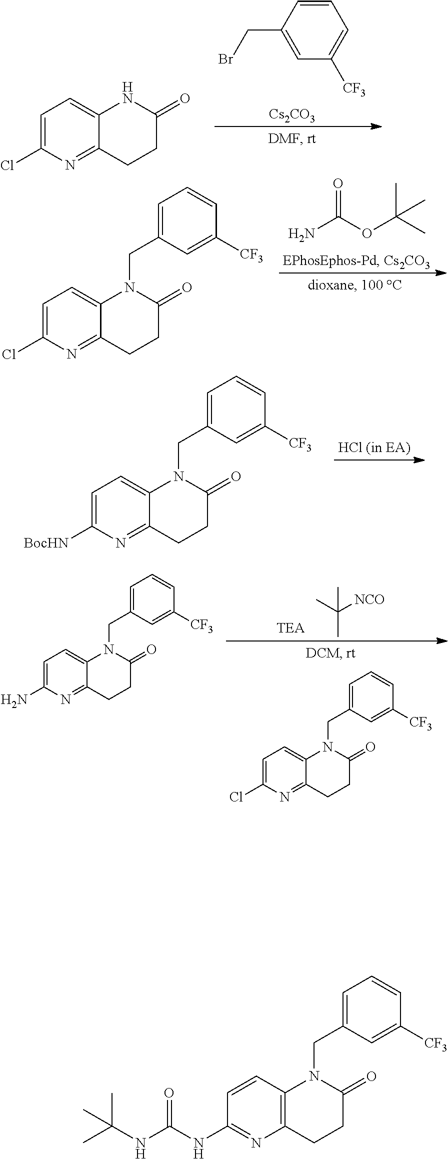

### 6-chloro-1-(3-(trifluoromethyl)benzyl)-3,4-dihydro-1,5-naphthyridin-2(1H)-one

To a stirred solution of 6-chloro-3,4-dihydro-1H-1,5-naphthyridin-2-one (300 mg, 1.643 mmol, 1 equiv) in anhydrous dimethylformamide (5 mL) was added 1-(bromomethyl)-3-(trifluoromethyl)benzene (432 mg, 1.807 mmol, 1.1 equiv) and Cs₂CO₃ (1.6 g, 4.929 mmol, 3 equiv) at rt and stirred for 2 h. After completion of reaction, the reaction mixture was quenched by water (20 mL) and extracted with EA (3×20 mL). The combined organic extracts were washed with brine (20 mL), dried over anhydrous Na₂SO₄ and concentrated under vacuum. The crude product was purified by flash chromatography with PE/EA (3:1) to afford 6-chloro-1-(3-(trifluoromethyl)benzyl)-3,4-dihydro-1,5-naphthyridin-2(1H)-one (390 mg, 69.67%) as a light-yellow solid.

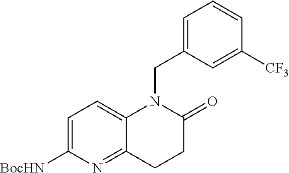 [SMILES: CC(C)(C)OC(=O)Nc1ccc2c(n1)CCC(=O)N2Cc1cccc(C(F)(F)F)c1]

### tert-butyl (6-oxo-5-(3-(trifluoromethyl)benzyl)-5,6,7,8-tetrahydro-1,5-naphthyridin-2-yl)carbamate

A solution of 6-chloro-1-(3-(trifluoromethyl)benzyl)-3,4-dihydro-1,5-naphthyridin-2(1H)-one (390 mg, 1.145 mmol, 1 equiv) in 1,4-dioxane (5 mL) was treated with tert-butyl carbamate (269 mg, 2.290 mmol, 2 equiv), Cs₂CO₃ (1.11 g, 3.435 mmol, 3 equiv), EPHOS (61 mg, 0.115 mmol, 0.1 equiv) and EPhos Pd G4 (105 mg, 0.115 mmol, 0.1 equiv) for overnight at 100° C. under nitrogen atmosphere. The reaction mixture was quenched by water (20 mL) and extracted with EA (3×20 mL). The combined organic extracts were washed with brine (20 mL), dried over anhydrous Na₂SO₄ and concentrated under vacuum. The crude product was purified by flash chromatography with PE/EA (3:1) to afford tert-butyl (6-oxo-5-(3-(trifluoromethyl)benzyl)-5,6,7,8-tetrahydro-1,5-naphthyridin-2-yl)carbamate (0.5 g, 96.01%).

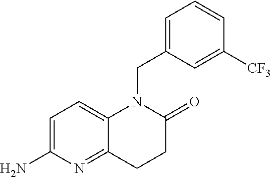 [SMILES: Nc1ccc2c(n1)CCC(=O)N2Cc1cccc(C(F)(F)F)c1]

### 6-amino-1-(3-(trifluoromethyl)benzyl)-3,4-dihydro-1,5-naphthyridin-2(1H)-one

To a stirred solution of tert-butyl (6-oxo-5-(3-(trifluoromethyl)benzyl)-5,6,7,8-tetrahydro-1,5-naphthyridin-2-yl)carbamate (350 mg, 0.831 mmol, 1 equiv) in HCl (4N in EA, 10 mL) at rt. The reaction was stirred for 2 h at rt. The resulting mixture was concentrated under reduced pressure to afford 6-amino-1-(3-(trifluoromethyl)benzyl)-3,4-dihydro-1,5-naphthyridin-2(1H)-one (300 mg, 112.42%) as a white solid.

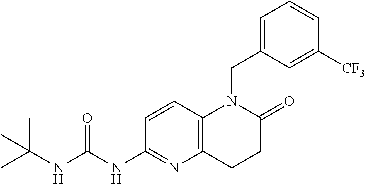 [SMILES: CC(C)(C)NC(=O)Nc1ccc2c(n1)CCC(=O)N2Cc1cccc(C(F)(F)F)c1]

### 1-(tert-butyl)-3-(6-oxo-5-(3-(trifluoromethyl)benzyl)-5,6,7,8-tetrahydro-1,5-naphthyridin-2-yl)urea

A solution of 6-amino-1-(3-(trifluoromethyl)benzyl)-3,4-dihydro-1,5-naphthyridin-2(1H)-one (50 mg, 0.156 mmol, 1 equiv), TEA (79 mg, 0.780 mmol, 5 equiv) and 2-isocyanato-2-methylpropane (77 mg, 0.780 mmol, 5 equiv) in DCM (5 mL) was stirred for overnight at rt. The mixture was concentrated under vacuum. The residue was purified by silica gel column chromatography, eluted with EA (46%-57%) to afford crude products. The residue was purified by reversed-phase flash chromatography with the following conditions Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 40% B to 65% B in 9 min, 65% B; Wave Length: 254/220 nm; RT1(min): 8.0; Number Of Runs: 0 to afford 3-tert-butyl-1-(6-oxo-5-{[3-(trifluoromethyl)phenyl]methyl}-7,8-dihydro-1,5-naphthyridin-2-yl)urea (19.6 mg, 29.91%) as a white solid. MS (ESI):mass calcd. for C₂₁H₂₃F₃N₄O₂: 420.18 m/z, found 421.10 [M+H]⁺. ¹H NMR (400 MHz, DMSO-d₆) δ 8.89 (s, 1H), 7.91 (s, 1H), 7.66-7.58 (m, 2H), 7.54 (t, J=7.6 Hz, 1H), 7.48 (d, J=7.7 Hz, 1H), 7.32 (d, J=8.9 Hz, 1H), 7.13 (d, J=8.9 Hz, 1H), 5.18 (s, 2H), 2.96-3.04 (m, 2H), 2.81 (dd, J=8.8, 6.2 Hz, 2H), 1.30 (s, 9H).

### Example 275: (R)-1-(1-benzyl-3-(cyanomethyl)-2-oxo-1,2,3,4-tetrahydroquinolin-6-yl)-3-(tert-butyl)urea

### Synthetic Scheme

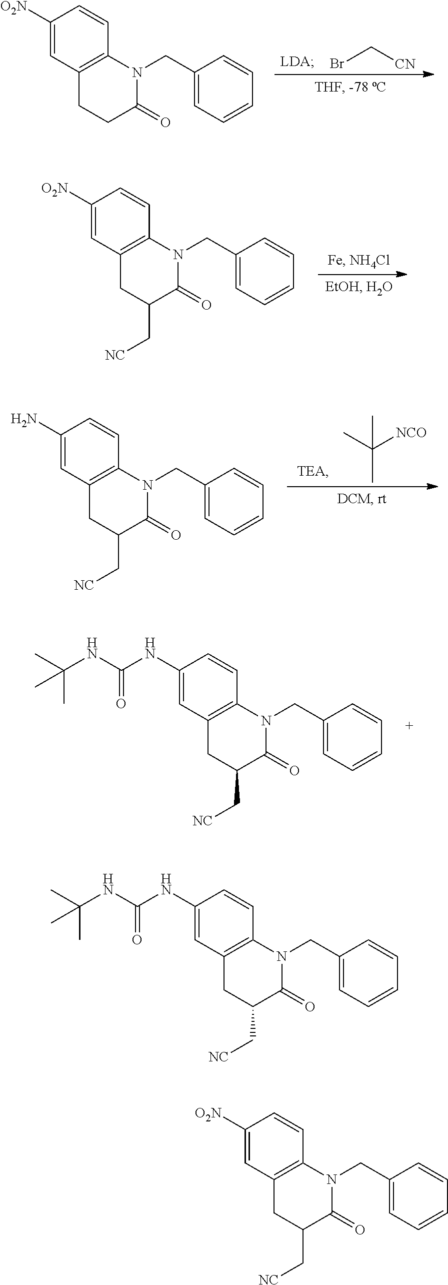

### 2-(1-benzyl-6-nitro-2-oxo-1,2,3,4-tetrahydroquinolin-3-yl)acetonitrile

3-In a 100-mL round bottom flask, to a solution of 1-benzyl-6-nitro-3,4-dihydroquinolin-2-one (300 mg, 1.063 mmol, 1 equiv) in tetrahydrofuran (5 mL) was added dropwise LDA (2M, in THF, 0.63 mL, 1.276 mmol, 1.2 equiv) at −78° C. under N₂ atmosphere. The reaction mixture was stirred at −78° C. for 20 mins. Then a solution of 2-bromoacetonitrile (153 mg, 1.276 mmol, 1.2 equiv) in 3 mL THF was added dropwise and the mixture was stirred for another 30 mins. The reaction was quenched with NH₄Cl (aq., 10 mL), and then the mixture were extracted with EtOAc (2×15 mL). The combined organic extracts were washed with brine (10 mL), dried over anhydrous Na₂SO₄ and concentrated under vacuum. The residue was purified by silica gel column chromatography, eluted with PE/EA (3/1) to afford 2-(1-benzyl 6-nitro-2-oxo-1,2,3,4-tetrahydroquinolin-3-yl)acetonitrile (180 mg, 52.71%) as a white solid.

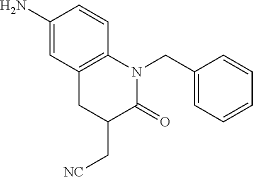 [SMILES: N#CCC1Cc2cc(N)ccc2N(Cc2ccccc2)C1=O]

### 2-(6-amino-1-benzyl-2-oxo-1,2,3,4-tetrahydroquinolin-3-yl)acetonitrile

A solution of 2-(1-benzyl-6-nitro-2-oxo-3,4-dihydroquinolin-3-yl)acetonitrile (180 mg, 0.560 mmol, 1 equiv) in EtOH (5 mL) and H₂O (0.5 mL) was added Fe (313 mg, 5.600 mmol, 10 equiv) and NH₄Cl (300 mg, 5.600 mmol, 10 equiv) at rt. The resulting mixture was stirred for overnight at 80° C. The reaction was quenched with H₂O (10 mL). The resulting mixture was extracted with EA (10 mL×3). The combined organic layers were washed with brine, dried over anhydrous Na₂SO₄. After filtration, the filtrate was concentrated under reduced pressure. The residue was purified by silica gel column chromatography, eluted with PE/EA (3/1) to afford 2-(6-amino-1-benzyl-2-oxo-1,2,3,4-tetrahydroquinolin-3-yl)acetonitrile (100 mg, 61.27%) as a brown solid. MS (ESI): mass calcd. for C₁₈H₁₇N₃O, 291.14 m/z, found 292.15 [M+H]⁺.

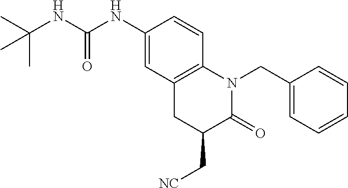 [SMILES: CC(C)(C)NC(=O)Nc1ccc2c(c1)C[C@H](CC#N)C(=O)N2Cc1ccccc1]

### (R)-1-(1-benzyl-3-(cyanomethyl)-2-oxo-1,2,3,4-tetrahydroquinolin-6-yl)-3-(tert-butyl)urea

A solution of 2-(6-amino-1-benzyl-2-oxo-1,2,3,4-tetrahydroquinolin-3-yl)acetonitrile (70 mg, 0.240 mmol, 1 equiv) in DCM (5 mL) was added 2-isocyanato-2-methylpropane (72 mg, 0.720 mmol, 3.0 equiv) and TEA (122 mg, 1.200 mmol, 5.0 equiv) at rt. The resulting mixture was stirred for overnight at rt. The reaction was quenched with H₂O (10 mL). The resulting mixture was extracted with EA (10 mL×3). The combined organic layers were washed with brine, dried over anhydrous Na₂SO₄. After filtration, the filtrate was concentrated under reduced pressure. The crude product was purified by silica gel column (PE/EA=2:1) and separated by Prep-Chiral-HPLC with the following conditions (Column: CHIRALPAK IF, 2*25 cm, 5 μm; Mobile Phase A: Hex(0.5% 2M NH₃-MeOH)—HPLC, Mobile Phase B: EtOH—HPLC; Flow rate: 20 mL/min; Gradient: 50% B to 50% B in 12 min; Wave Length: 220/254 nm; RT1(min): 6.534; RT2(min): 10.398; Sample Solvent: EtOH—HPLC; Injection Volume: 1.8 mL; Number Of Runs: 4) to afforded (R)-1-(1-benzyl-3-(cyanomethyl)-2-oxo-1,2,3,4-tetrahydroquinolin-6-yl)-3-(tert-butyl)urea (18.8 mg, 19.39%) as an off-white solid. MS (ESI): mass calcd. C₂₃H₂₆N₄O₂ for: 390.21 m/z, found 391.15 [M+H]⁺. ¹H NMR (400 MHz, DMSO-d₆) δ 8.19 (s, 1H), 7.29-7.35 (m, 3H), 7.24-7.28 (m, 2H), 7.19-7.21 (m, 1H), 7.01-7.06 (m, 1H), 6.79-6.86 (m, 1H), 5.97 (s, 1H), 5.03-5.20 (m, 2H), 3.05-3.15 (m, 1H), 2.83-3.02 (m, 4H), 1.26 (s, 9H). And to afford (S)-1-(1-benzyl-3-(cyanomethyl)-2-oxo-1,2,3,4-tetrahydroquinolin-6-yl)-3-(tert-butyl)urea (11.9 mg, 12.66%) as an off-white solid. MS (ESI): mass calcd. C₂₃H₂₆N₄O₂ for: 390.21m/z, found 391.15 [M+H]⁺. ¹H NMR (400 MHz, DMSO-d₆) δ 8.19 (s, 1H), 7.29-7.35 (m, 3H), 7.24-7.28 (m, 2H), 7.19-7.21 (m, 1H), 7.01-7.06 (m, 1H), 6.79-6.86 (m, 1H), 5.97 (s, 1H), 5.03-5.20 (m, 2H), 3.05-3.15 (m, 1H), 2.83-3.02 (m, 4H), 1.26 (s, 9H).

### Example 276: (S)-1-(1-benzyl-3-(cyanomethyl)-2-oxo-1,2,3,4-tetrahydroquinolin-6-yl)-3-(tert-butyl)urea

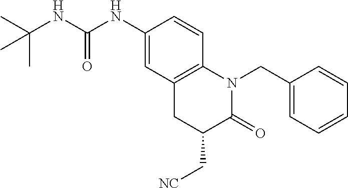 [SMILES: CC(C)(C)NC(=O)Nc1ccc2c(c1)C[C@@H](CC#N)C(=O)N2Cc1ccccc1]

### (S)-1-(1-benzyl-3-(cyanomethyl)-2-oxo-1,2,3,4-tetrahydroquinolin-6-yl)-3-(tert-butyl)urea

A solution of 2-(6-amino-1-benzyl-2-oxo-1,2,3,4-tetrahydroquinolin-3-yl)acetonitrile (70 mg, 0.240 mmol, 1 equiv) in DCM (5 mL) was added 2-isocyanato-2-methylpropane (72 mg, 0.720 mmol, 3.0 equiv) and TEA (122 mg, 1.200 mmol, 5.0 equiv) at rt. The resulting mixture was stirred for overnight at rt. The reaction was quenched with H₂O (10 mL). The resulting mixture was extracted with EA (10 mL×3). The combined organic layers were washed with brine, dried over anhydrous Na₂SO₄. After filtration, the filtrate was concentrated under reduced pressure. The crude product was purified by silica gel column (PE/EA=2:1) and separated by Prep-Chiral-HPLC with the following conditions (Column: CHIRALPAK IF, 2*25 cm, 5 μm; Mobile Phase A: Hex(0.5% 2M NH₃-MeOH)—HPLC, Mobile Phase B: EtOH—HPLC; Flow rate: 20 mL/min; Gradient: 50% B to 50% B in 12 min; Wave Length: 220/254 nm; RT1(min): 6.534; RT2(min): 10.398; Sample Solvent: EtOH—HPLC; Injection Volume: 1.8 mL; Number Of Runs: 4) to afforded (R)-1-(1-benzyl-3-(cyanomethyl)-2-oxo-1,2,3,4-tetrahydroquinolin-6-yl)-3-(tert-butyl)urea (18.8 mg, 19.39%) as an off-white solid. MS (ESI): mass calcd. C₂₃H₂₆N₄O₂ for: 390.21 m/z, found 391.15 [M+H]⁺. ¹H NMR (400 MHz, DMSO-d₆) δ 8.19 (s, 1H), 7.29-7.35 (m, 3H), 7.24-7.28 (m, 2H), 7.19-7.21 (m, 1H), 7.01-7.06 (m, 1H), 6.79-6.86 (m, 1H), 5.97 (s, 1H), 5.03-5.20 (m, 2H), 3.05-3.15 (m, 1H), 2.83-3.02 (m, 4H), 1.26 (s, 9H). And to afford (S)-1-(1-benzyl-3-(cyanomethyl)-2-oxo-1,2,3,4-tetrahydroquinolin-6-yl)-3-(tert-butyl)urea (11.9 mg, 12.66%) as an off-white solid. MS (ESI): mass calcd. C₂₃H₂₆N₄O₂ for: 390.21m/z, found 391.15 [M+H]⁺. ¹H NMR (400 MHz, DMSO-d₆) δ 8.19 (s, 1H), 7.29-7.35 (m, 3H), 7.24-7.28 (m, 2H), 7.19-7.21 (m, 1H), 7.01-7.06 (m, 1H), 6.79-6.86 (m, 1H), 5.97 (s, 1H), 5.03-5.20 (m, 2H), 3.05-3.15 (m, 1H), 2.83-3.02 (m, 4H), 1.26 (s, 9H).

### Example 277: (R)-1-(4-benzyl-2-ethyl-3-oxo-3,4-dihydro-2H-benzo[b][1,4]oxazin-7-yl)-3-(tert-butyl)urea

### Synthetic Scheme

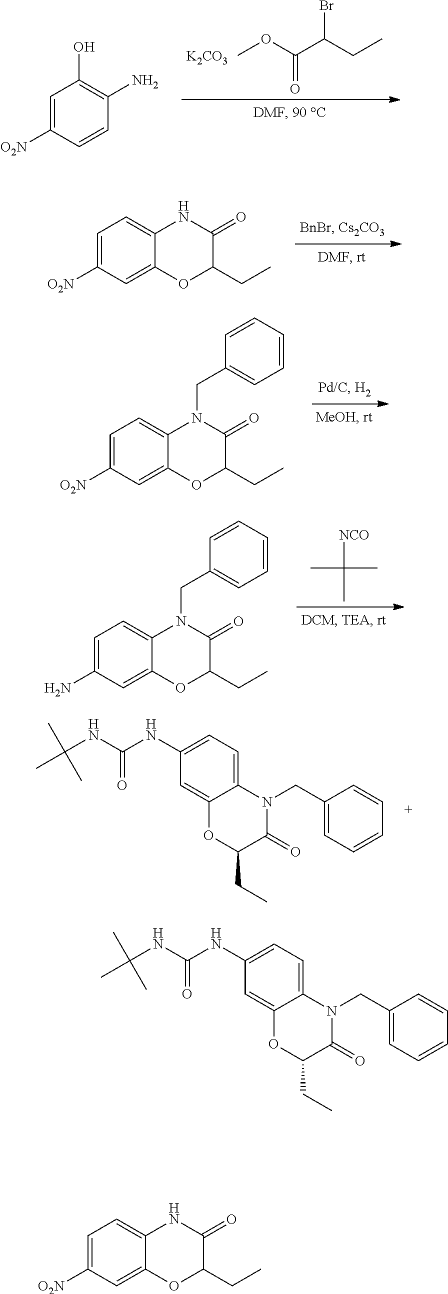

### 2-ethyl-7-nitro-2H-benzo[b][1,4]oxazin-3(4H)-one

To a stirred solution of 2-amino-5-nitrophenol (1 g, 6.488 mmol, 1 equiv) in anhydrous dimethylformamide (30 mL) was added K₂CO₃ (2.71 g, 19.464 mmol, 3 equiv) and methyl 2-bromobutanoate (1.41 g, 7.786 mmol, 1.2 equiv) at 0° C. and stirred for 30 min and the mixture is stirred for overnight at 90° C. The reaction mixture was quenched by addition of water (15 mL). The aqueous layer was extracted with ethyl acetate (3×30 mL). The combined organic phase was washed with brine (20 mL), dried over anhydrous sodium sulfate filtered and concentrated under reduced pressure. The residue was purified by silica gel column, eluted with PE/EA (3:1) to give 2-ethyl-7-nitro-2H-benzo[b][1,4]oxazin-3(4H)-one (1.3 g, 90.17%) as a yellow solid.

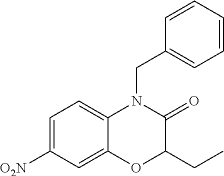 [SMILES: CCC1Oc2cc([N+](=O)[O-])ccc2N(Cc2ccccc2)C1=O]

### 4-benzyl-2-ethyl-7-nitro-2H-benzo[b][1,4]oxazin-3(4H)-one

To a stirred solution of To a stirred solution of 2-amino-5-nitrophenol (0.5 g, 2.25 mmol, 1 equiv) in anhydrous dimethylformamide (6 mL) was added Cs₂CO₃ (1.5 g, 4.50 mmol, 2 equiv) and BnBr (460 mg, 2.70 mmol, 1.2 equiv) at rt and stirred for 16 h. The reaction mixture was quenched by addition of water (15 mL). The aqueous layer was extracted with ethyl acetate (3×30 mL). The combined organic phase was washed with brine (20 mL), dried over anhydrous sodium sulfate filtered and concentrated under reduced pressure. The residue was purified by silica gel column, eluted with PE/EA (3:1) to give 4-benzyl-2-ethyl-7-nitro-2H-benzo[b][1,4]oxazin-3(4H)-one (0.5 g, 71.43%) as a yellow solid.

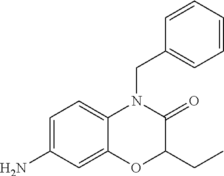 [SMILES: CCC1Oc2cc(N)ccc2N(Cc2ccccc2)C1=O]

### 7-amino-4-benzyl-2-ethyl-2H-benzo[b][1,4]oxazin-3(4H)-one

To a solution of 4-benzyl-2-ethyl-7-nitro-2H-benzo[b][1,4]oxazin-3(4H)-one (200 mg, 0.640 mmol, 1 equiv) in 15 mL MeOH was added Pd/C (10%, 0.1 g) under nitrogen atmosphere. The mixture was hydrogenated at room temperature for 1 h under hydrogen atmosphere using a hydrogen balloon, filtered through a Celite pad and concentrated under reduced pressure. This resulted in 7-7-amino-4-benzyl-2-ethyl-2H-benzo[b][1,4]oxazin-3(4H)-one (180 mg, 99.56%) as a white solid.

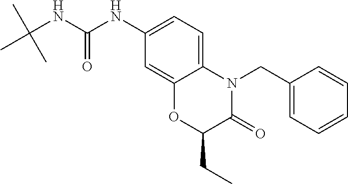 [SMILES: CC[C@H]1Oc2cc(NC(=O)NC(C)(C)C)ccc2N(Cc2ccccc2)C1=O]

### (R)-1-(4-benzyl-2-ethyl-3-oxo-3,4-dihydro-2H-benzo[b][1,4]oxazin-7-yl)-3-(tert-butyl)urea

A solution of 7-amino-4-benzyl-2-ethyl-2H-benzo[b][1,4]oxazin-3(4H)-one (150 mg, 0.531 mmol, 1 equiv), TEA (269 mg, 2.655 mmol, 5 equiv) and 2-isocyanato-2-methylpropane (263 mg, 2.655 mmol, 5 equiv) in DCM (10 mL) was stirred for overnight at rt. The reaction mixture was quenched by addition of water (15 mL). The aqueous layer was extracted with DCM (3×20 mL). The combined organic phase was washed with brine (20 mL), dried over anhydrous sodium sulfate filtered and concentrated under reduced pressure. The residue was purified by silica gel column, eluted with PE/EA (2:1) to give crude product. The residue was purified by Chiral HPLC with the following conditions Column: CHIRALPAK ID, 2*25 cm, 5 μm; Mobile Phase A: Hex(0.5% 2M NH₃-MeOH)—HPLC, Mobile Phase B: EtOH—HPLC; Flow rate: 20 mL/min; Gradient: 15% B to 15% B in 16 min; Wave Length: 220/254 nm; RT1(min): 9.531; RT2(min): 12.768; Sample Solvent: EtOH—HPLC; Injection Volume: 0.8 mL; Number Of Runs: 6 to afford (R)-1-(4-benzyl-2-ethyl-3-oxo-3,4-dihydro-2H-benzo[b][1,4]oxazin-7-yl)-3-(tert-butyl)urea (15.5 mg, 7.64%) as a white solid. MS (ESI): mass calcd. for C₂₂H₂₇N₃O₃: 381.21 m/z, found 382.20 [M+H]⁺. ¹H NMR (300 MHz, DMSO-d₆) δ 8.20 (d, J=2.8 Hz, 1H), 7.22-7.33 (m, 6H), 6.85 (dd, J=8.7, 2.8 Hz, 1H), 6.76-6.66 (m, 1H), 5.94 (d, J=2.8 Hz, 1H), 5.10 (t, J=3.5 Hz, 2H), 4.63-4.65 (m, 1H), 1.78-1.91 (m, 2H), 1.26 (s, 9H), 1.04 (t, J=7.4 Hz, 3H). And (S)-1-(4-benzyl-2-ethyl-3-oxo-3,4-dihydro-2H-benzo[b][1,4]oxazin-7-yl)-3-(tert-butyl)urea (13.4 mg, 6.60%) as a white solid. MS (ESI): mass calcd. for C₂₂H₂₇N₃O₃: 381.21 m/z, found 382.20 [M+H]⁺. ¹H NMR (300 MHz, DMSO-d₆) δ 8.20 (d, J=2.8 Hz, 1H), 7.22-7.33 (m, 6H), 6.85 (dd, J=8.7, 2.8 Hz, 1H), 6.76-6.66 (m, 1H), 5.94 (d, J=2.8 Hz, 1H), 5.10 (t, J=3.5 Hz, 2H), 4.63-4.65 (m, 1H), 1.78-1.91 (m, 2H), 1.26 (s, 9H), 1.04 (t, J=7.4 Hz, 3H).

### Example 278: (S)-1-(4-benzyl-2-ethyl-3-oxo-3,4-dihydro-2H-benzo[b][1,4]oxazin-7-yl)-3-(tert-butyl)urea

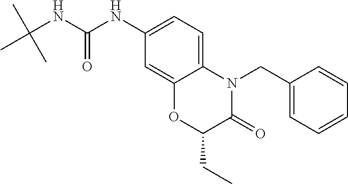 [SMILES: CC[C@@H]1Oc2cc(NC(=O)NC(C)(C)C)ccc2N(Cc2ccccc2)C1=O]

### (S)-1-(4-benzyl-2-ethyl-3-oxo-3,4-dihydro-2H-benzo[b][1,4]oxazin-7-yl)-3-(tert-butyl)urea

A solution of 7-amino-4-benzyl-2-ethyl-2H-benzo[b][1,4]oxazin-3(4H)-one (150 mg, 0.531 mmol, 1 equiv), TEA (269 mg, 2.655 mmol, 5 equiv) and 2-isocyanato-2-methylpropane (263 mg, 2.655 mmol, 5 equiv) in DCM (10 mL) was stirred for overnight at rt. The reaction mixture was quenched by addition of water (15 mL). The aqueous layer was extracted with DCM (3×20 mL). The combined organic phase was washed with brine (20 mL), dried over anhydrous sodium sulfate filtered and concentrated under reduced pressure. The residue was purified by silica gel column, eluted with PE/EA (2:1) to give crude product. The residue was purified by Chiral HPLC with the following conditions Column: CHIRALPAK ID, 2*25 cm, 5 μm; Mobile Phase A: Hex(0.5% 2M NH₃-MeOH)—HPLC, Mobile Phase B: EtOH—HPLC; Flow rate: 20 mL/min; Gradient: 15% B to 15% B in 16 min; Wave Length: 220/254 nm; RT1(min): 9.531; RT2(min): 12.768; Sample Solvent: EtOH—HPLC; Injection Volume: 0.8 mL; Number Of Runs: 6 to afford (R)-1-(4-benzyl-2-ethyl-3-oxo-3,4-dihydro-2H-benzo[b][1,4]oxazin-7-yl)-3-(tert-butyl)urea (15.5 mg, 7.64%) as a white solid. MS (ESI): mass calcd. for C₂₂H₂₇N₃O₃: 381.21 m/z, found 382.20 [M+H]⁺. ¹H NMR (300 MHz, DMSO-d₆) δ 8.20 (d, J=2.8 Hz, 1H), 7.22-7.33 (m, 6H), 6.85 (dd, J=8.7, 2.8 Hz, 1H), 6.76-6.66 (m, 1H), 5.94 (d, J=2.8 Hz, 1H), 5.10 (t, J=3.5 Hz, 2H), 4.63-4.65 (m, 1H), 1.78-1.91 (m, 2H), 1.26 (s, 9H), 1.04 (t, J=7.4 Hz, 3H). And (S)-1-(4-benzyl-2-ethyl-3-oxo-3,4-dihydro-2H-benzo[b][1,4]oxazin-7-yl)-3-(tert-butyl)urea (13.4 mg, 6.60%) as a white solid. MS (ESI): mass calcd. for C₂₂H₂₇N₃O₃: 381.21 m/z, found 382.20 [M+H]⁺. ¹H NMR (300 MHz, DMSO-d₆) δ 8.20 (d, J=2.8 Hz, 1H), 7.22-7.33 (m, 6H), 6.85 (dd, J=8.7, 2.8 Hz, 1H), 6.76-6.66 (m, 1H), 5.94 (d, J=2.8 Hz, 1H), 5.10 (t, J=3.5 Hz, 2H), 4.63-4.65 (m, 1H), 1.78-1.91 (m, 2H), 1.26 (s, 9H), 1.04 (t, J=7.4 Hz, 3H).

### Example 279: 1-(1-benzyl-5-cyano-2-oxo-1,2,3,4-tetrahydroquinolin-6-yl)-3-(tert-butyl)urea

### Synthetic Scheme

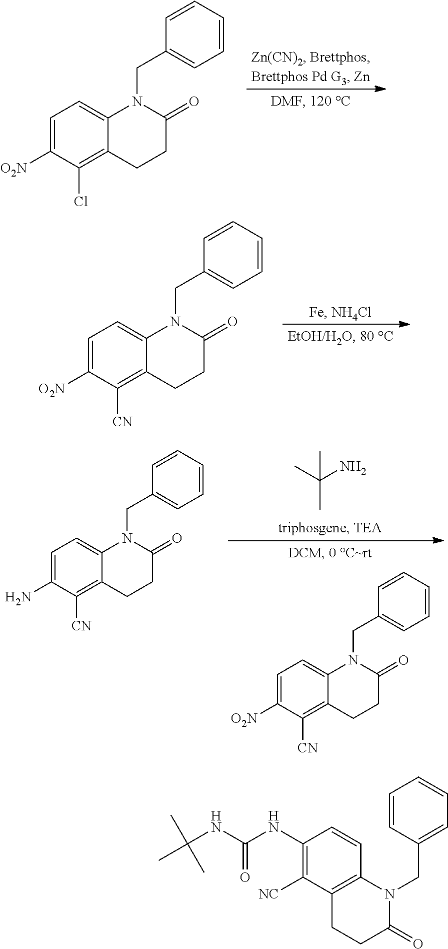

### 1-benzyl-6-nitro-2-oxo-1,2,3,4-tetrahydroquinoline-5-carbonitrile

To a solution of 1-benzyl-5-chloro-6-nitro-3,4-dihydroquinolin-2-one (180 mg, 0.568 mmol, 1 equiv) in dimethylformamide (2 mL) was added zinc (5.57 mg, 0.085 mmol, 0.15 equiv), zinc cyanide (80.07 mg, 0.682 mmol, 1.2 equiv), Brettphos Pd G₃ (103.03 mg, 0.114 mmol, 0.2 equiv) and Brettphos (61.01 mg, 0.114 mmol, 0.2 equiv). The reaction was stirred at 120° C. under N₂ for 3 h. After cooling down to rt, the reaction was quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give 1-benzyl-6-nitro-2-oxo-3,4-dihydroquinoline-5-carbonitrile (77 mg, 44.09%) as a white solid. LC/MS (ESI): mass calcd. for C₁₇H₁₃N₃O₃: 307.10 m/z, found: 308.10 [M+H]⁺.

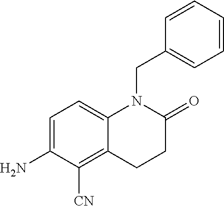 [SMILES: N#Cc1c(N)ccc2c1CCC(=O)N2Cc1ccccc1]

### 6-amino-1-benzyl-2-oxo-1,2,3,4-tetrahydroquinoline-5-carbonitrile

To a solution of 1-benzyl-6-nitro-2-oxo-3,4-dihydroquinoline-5-carbonitrile (77 mg, 0.251 mmol, 1 equiv) in ethyl alcohol (20 mL) and water (2 mL) was added iron (140 mg, 2.507 mmol, 10.01 equiv) and NH₄Cl (133 mg, 2.486 mmol, 9.92 equiv). The reaction was stirred at 60° C. for 3 h. After cooling down to rt, the reaction was quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 6-amino-1-benzyl-2-oxo-3,4-dihydroquinoline-5-carbonitrile (22 mg, 31.66%) as a yellow solid. LC/MS (ESI): mass calcd. for C₁₇H₁₅N₃O: 277.12 m/z, found: 278.10 [M+H]⁺.

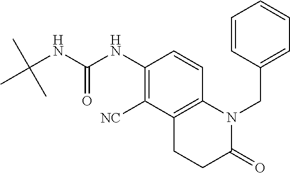 [SMILES: CC(C)(C)NC(=O)Nc1ccc2c(c1C#N)CCC(=O)N2Cc1ccccc1]

### 1-(1-benzyl-5-cyano-2-oxo-1,2,3,4-tetrahydroquinolin-6-yl)-3-(tert-butyl)urea

To a solution of 6-amino-1-benzyl-2-oxo-3,4-dihydroquinoline-5-carbonitrile (80 mg, 0.288 mmol, 1 equiv) in DCM (4 mL) was added erbumine (42.20 mg, 0.576 mmol, 2 equiv). The reaction was stirred at 0° C. under N₂ for 1 h. The reaction was added TEA (87.57 mg, 0.864 mmol, 3 equiv) and stirred at rt under N₂ for 1 h. The reaction was added 2-methylpropan-2-amine (42.20 mg, 0.576 mmol, 2 equiv) and stirred at rt under N₂ for 1 h. Quenched with water (10 mL) and extracted with DCM (3×10 mL). The combined extracts were washed with water, brine, dried over Na₂SO₄, filtered and concentrated. The residue was purified by preparative HPLC using a Aeris PEPTIDE 5 um XB-C18 Axia 21.2 mm×250 mm, 5 μm column (eluent: 43% to 57% (v/v) CH₃CN and H₂O with 10 mmol/L NH₄HCO₃) to afford the title compound 1-(1-benzyl-5-cyano-2-oxo-3,4-dihydroquinolin-6-yl)-3-tert-butylurea (12.5 mg, 11.48%) as a white solid. LC/MS (ESI): mass calcd. for C₂₂H₂₄N₄O₂:376.19 m/z, found: 377.05 [M+H]⁺. ¹H NMR (300 MHz, DMSO-d6) δ 8.19 (s, 1H), 7.71-7.74 (m, 1H), 7.11-7.36 (m, 6H), 6.80 (s, 1H), 5.14 (s, 2H), 3.06-3.11 (m, 2H), 2.72-2.77 (m, 2H), 1.27 (s, 9H).

### Example 280: (R)-1-(1-benzyl-3-methyl-2-oxo-2,3-dihydro-1H-pyrido[2,3-b][1,4]oxazin-6-yl)-3-(tert-butyl)urea

### Synthetic Scheme

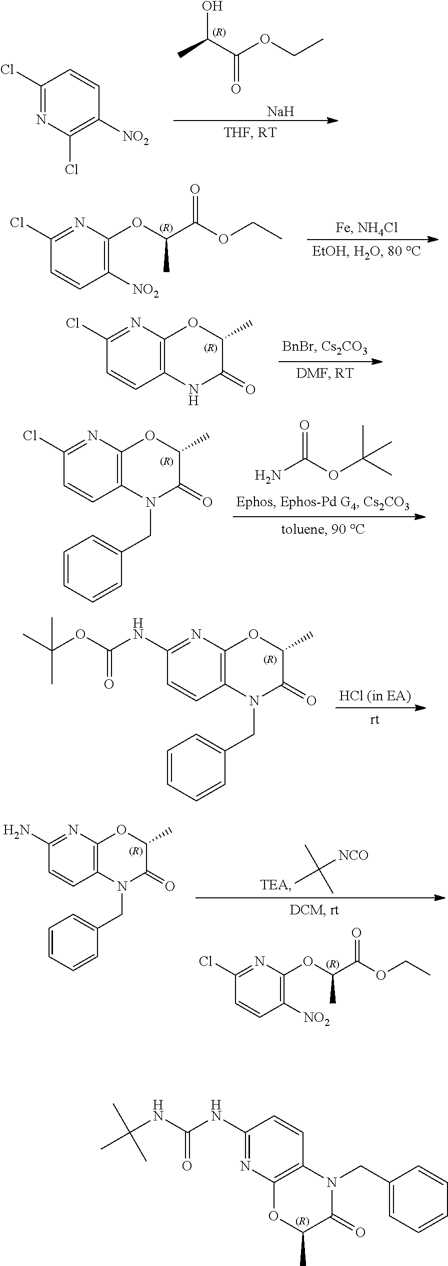

### ethyl (R)-2-((6-chloro-3-nitropyridin-2-yl)oxy)propanoate

To a solution of 2,6-dichloro-3-nitropyridine (2.0 g, 10.364 mmol, 1 equiv) in tetrahydrofuran (20 mL) was added 60% NaH (0.5 g, 12.437 mmol, 1.2 equiv) at 0° C. The mixture was stirred for 15 min. ethyl lactate (1.47 g, 12.437 mmol, 1.2 equiv) was added and the mixture was allowed to warm to RT and stirred for 2 h. The reaction mixture was quenched by water (10 mL) and extracted with EA (3×20 mL). The combined organic layers were washed with NaCl (20 mL), dried over anhydrous Na₂SO₄. After filtration, the filtrate was concentrated under reduced pressure. The residue was purified by silica gel column chromatography, eluted with PE/EA (5/1) to afford ethyl (R)-2-((6-chloro-3-nitropyridin-2-yl)oxy)propanoate (2.0 g, 70.26%) as a white solid. MS (ESI): mass calcd. for C₁₀H₁₁ClN₂O₅, 274.04 m/z, found 275.05 [M+H]⁺.

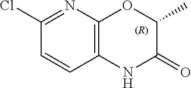

### (R)-6-chloro-3-methyl-1H-pyrido[2,3-b][1,4]oxazin-2(3H)-one

A solution of ethyl (R)-2-((6-chloro-3-nitropyridin-2-yl)oxy)propanoate (500 mg, 1.820 mmol, 1 equiv) in EtOH (10 mL) and H₂O (1 mL) was added Fe (2.0 g, 36.400 mmol, 20 equiv) and NH₄Cl (1.9 g, 36.400 mmol, 20 equiv) at rt. The resulting mixture was stirred for overnight at 80° C. The reaction was quenched with water (10 mL). The aqueous layer was extracted with EtOAc (3×10 mL). The residue was purified by silica gel column chromatography, eluted with PE/EA (3:1) to afford (R)-6-chloro-3-methyl-1H-pyrido[2,3-b][1,4]oxazin-2(3H)-one (300 mg, 82.97%) as a brown solid. MS (ESI): mass calcd. for C₈H₇ClN₂O₂, 198.02 m/z, found 199.10 [M+H]⁺.

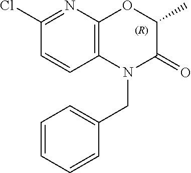

### (R)-1-benzyl-6-chloro-3-methyl-1H-pyrido[2,3-b][1,4]oxazin-2(3H)-one

A solution of (R)-6-chloro-3-methyl-1H-pyrido[2,3-b][1,4]oxazin-2(3H)-one (300 mg, 1.510 mmol, 1 equiv) in DMF (5 mL) was added BnBr (775 mg, 4.530 mmol, 3.0 equiv) and Cs₂CO₃ (2.0 g, 6.040 mmol, 4.0 equiv) at rt. The resulting mixture was stirred for 2 h at rt. The reaction was quenched with water (20 mL). The resulting mixture was extracted with EA (3×20 mL). The combined organic layers were washed with NaCl (aq., 3×10 mL), dried over anhydrous Na₂SO₄. After filtration, the filtrate was concentrated under reduced pressure. The residue was purified by silica gel column chromatography, eluted with PE/EA (3/1) to afford (R)-1-benzyl-6-chloro-3-methyl-1H-pyrido[2,3-b][1,4]oxazin-2(3H)-one (300 mg, 68.79%) as a white solid. MS (ESI): mass calcd. for C₁₅H₁₃ClN₂O₂, 288.07 m/z, found 289.15 [M+H]⁺.

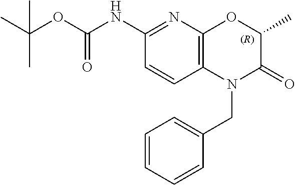

### tert-butyl (R)-(1-benzyl-3-methyl-2-oxo-2,3-dihydro-1H-pyrido[2,3-b][1,4]oxazin-6-yl)carbamate

A solution of (R)-1-benzyl-6-chloro-3-methyl-1H-pyrido[2,3-b][1,4]oxazin-2(3H)-one (150 mg, 0.520 mmol, 1 equiv) in dioxane (5 mL) was added tert-butyl carbamate (73 mg, 0.624 mmol, 1.2 equiv), Cs₂CO₃ (338 mg, 1.040 mmol, 2.0 equiv), Ephos (55.5 mg, 0.104 mmol, 0.2 equiv) and Ephos Pd G4 (48.0 mg, 0.052 mmol, 0.1 equiv) at rt. The resulting mixture was stirred for overnight at 90° C. The resulting was concentrated under reduced pressure. The residue was purified by silica gel column chromatography, eluted with PE/EA (3/1) to afford tert-butyl (R)-(1-benzyl-3-methyl-2-oxo-2,3-dihydro-1H-pyrido[2,3-b][1,4]oxazin-6-yl)carbamate (110 mg, 57.32%) as an off-white solid. MS (ESI): mass calcd. for C₂₀H₂₃N₃O₄, 369.17 m/z, found 370.10 [M+H]⁺.

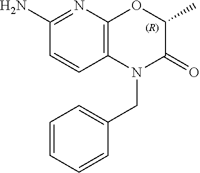

### (R)-6-amino-1-benzyl-3-methyl-1H-pyrido[2,3-b][1,4]oxazin-2(3H)-one

A solution of tert-butyl (R)-(1-benzyl-3-methyl-2-oxo-2,3-dihydro-1H-pyrido[2,3-b][1,4]oxazin-6-yl)carbamate (100 mg, 0.271 mmol, 1 equiv) in 4N HCl (in EA, 10 mL) at rt. The resulting mixture was stirred for 2 h at rt. The resulting mixture was concentrated under reduced pressure to afford (R)-6-amino-1-benzyl-3-methyl-1H-pyrido[2,3-b][1,4]oxazin-2(3H)-one (70 mg, 96.02%) was used in the next step directly without further purification. MS (ESI): mass calcd. for C₁₅H₁₅N₃O₂, 269.12 m/z, found 270.20 [M+H]⁺.

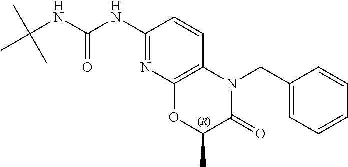

### (R)-1-(1-benzyl-3-methyl-2-oxo-2,3-dihydro-1H-pyrido[2,3-b][1,4]oxazin-6-yl)-3-(tert-butyl)urea

A solution of (R)-6-amino-1-benzyl-3-methyl-1H-pyrido[2,3-b][1,4]oxazin-2(3H)-one (80 mg, 0.297 mmol, 1 equiv) in DCM (5 mL) was added 2-isocyanato-2-methylpropane (294 mg, 2.970 mmol, 10 equiv) and TEA (300 mg, 2.970 mmol, 10 equiv) at rt. The resulting mixture was stirred for overnight at rt. The reaction was quenched with water (10 mL). The aqueous layer was extracted with EtOAc (3×10 mL). The residue was purified by silica gel column chromatography, eluted with PE/EA (1:1) and C18 column to afford (R)-1-(1-benzyl-3-methyl-2-oxo-2,3-dihydro-1H-pyrido[2,3-b][1,4]oxazin-6-yl)-3-(tert-butyl)urea (28.0 mg, 25.51%) as a white solid. MS (ESI): mass calcd. C20H24N4O3 for: 368.18 m/z, found 369.20 [M+H]⁺. ¹H NMR (400 MHz, DMSO-d₆) δ 8.72 (s, 1H), 7.36-7.43 (m, 1H), 7.29-7.35 (m, 2H), 7.22-7.29 (m, 3H), 7.16-7.22 (m, 1H), 7.08-7.12 (m, 1H), 4.79-5.19 (m, 3H), 1.53 (d, J=6.8 Hz, 3H), 1.29 (s, 9H).

### Example 281: 1-(tert-butyl)-3-(5-(3-chlorobenzyl)-6-oxo-5,6,7,8-tetrahydro-1,5-naphthyridin-2-yl)urea

### Synthetic Scheme

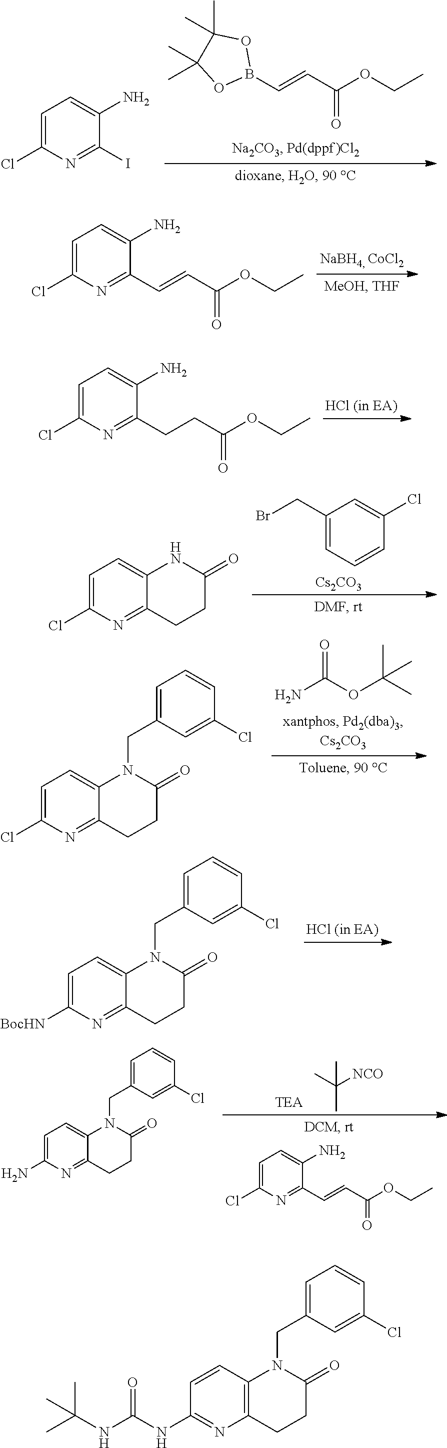

### ethyl (E)-3-(3-amino-6-chloropyridin-2-yl)acrylate

A solution of 6-chloro-2-iodopyridin-3-amine (2.3 g, 9.039 mmol, 1 equiv) ethyl (2E)-3-(4,4,5,5-tetramethyl-1,3,2-dioxaborolan-2-yl)prop-2-enoate (2.0 g, 9.039 mmol, 1 equiv), Na₂CO₃ (2.9 g, 27.117 mmol, 3 equiv) and Pd(dppf)Cl₂ (0.66 g, 0.904 mmol, 0.1 equiv) in water (3 mL) and 1,4-dioxane (30 mL) was stirred for overnight at 90° C. under N₂ atmosphere. The mixture was allowed to cool down to rt. The reaction mixture was quenched by water (50 mL) and extracted with EA (3×100 mL). The combined organic extracts were washed with brine (50 mL), dried over anhydrous Na₂SO₄ and concentrated under vacuum. The crude product was purified by flash chromatography with PE/EA (3:1) to afford ethyl (E)-3-(3-amino-6-chloropyridin-2-yl)acrylate (1.5 g, 73.22%) as a yellow solid.

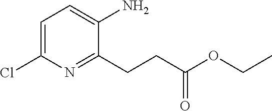

### ethyl 3-(3-amino-6-chloropyridin-2-yl)propanoate

A solution of ethyl (E)-3-(3-amino-6-chloropyridin-2-yl)acrylate (1.4 g, 6.177 mmol, 1 equiv) in methanol (10 mL) and tetrahydrofuran (20 mL) was treated with cobalt(II) chloride (0.40 g, 3.088 mmol, 0.5 equiv) for 30 min at 0° C. under nitrogen atmosphere followed by the addition of NaBH₄ (1.17 g, 30.885 mmol, 5 equiv) in portions at 0° C. The resulting mixture was stirred for additional 1 h at rt. The reaction mixture was quenched by water (20 mL) and extracted with EA (3×30 mL). The combined organic extracts were washed with brine (20 mL), dried over anhydrous Na₂SO₄ and concentrated under vacuum. The crude product was purified by flash chromatography with PE/EA (2:1) to afford ethyl 3-(3-amino-6-chloropyridin-2-yl)propanoate (1.2 g, 84.96%) as a white solid. MS (ESI): mass calcd. for C₁₀H₁₃ClN₂O₂, 228.07 m/z, found 229.10 [M+H]⁺.

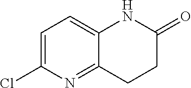 [SMILES: O=C1CCc2nc(Cl)ccc2N1]

### 6-chloro-3,4-dihydro-1,5-naphthyridin-2(1H)-one

To a stirred solution of ethyl 3-(3-amino-6-chloropyridin-2-yl)propanoate (1.2 g, 5.248 mmol, 1 equiv) in EA (10 mL), HCl (4N, in EA, 20 mL) was added at rt. The reaction stirred for 2 h. The precipitated solids were collected by filtration and washed with Et₂₀ (3×50 mL). This resulted in 6-chloro-3,4-dihydro-1,5-naphthyridin-2(1H)-one (1.08 g, 112.71%) as a white solid. MS (ESI): mass calcd. for C₈H₇ClN₂O, 182.02 m/z, found 183.10 [M+H]⁺.

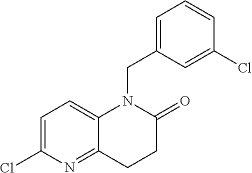 [SMILES: O=C1CCc2nc(Cl)ccc2N1Cc1cccc(Cl)c1]

### 6-chloro-1-(3-chlorobenzyl)-3,4-dihydro-1,5-naphthyridin-2(1H)-one

To a stirred solution of 6-chloro-3,4-dihydro-1,5-naphthyridin-2(1H)-one (300 mg, 1.643 mmol, 1 equiv) in anhydrous DMF (20 mL) was added 1-(bromomethyl)-3-chlorobenzene (371 mg, 1.807 mmol, 1.1 equiv) and Cs₂CO₃ (1.6 g, 4.929 mmol, 3 equiv) at rt and stirred for 2 h. The reaction mixture was quenched by water (10 mL) and extracted with EA (3×10 mL). The combined organic extracts were washed with brine (10 mL), dried over anhydrous Na₂SO₄ and concentrated under vacuum. The crude product was purified by flash chromatography with PE/EA (3:1) to afford 6-chloro-1-(3-chlorobenzyl)-3,4-dihydro-1,5-naphthyridin-2(1H)-one (293 mg, 58.06%) as a light-yellow solid. MS (ESI): mass calcd. for C₁₅H₁₂C₁₂N₂O, 306.03 m/z, found 307.00 [M+H]⁺.

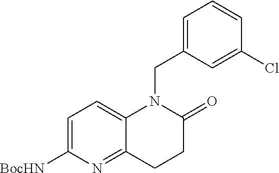 [SMILES: CC(C)(C)OC(=O)Nc1ccc2c(n1)CCC(=O)N2Cc1cccc(Cl)c1]

### tert-butyl (5-(3-chlorobenzyl)-6-oxo-5,6,7,8-tetrahydro-1,5-naphthyridin-2-yl)carbamate

To a stirred solution of 6-chloro-1-(3-chlorobenzyl)-3,4-dihydro-1,5-naphthyridin-2(1H)-one (210 mg, 0.684 mmol, 1 equiv) in anhydrous toluene (15 mL) was added Cs₂CO₃ (670 mg, 2.052 mmol, 3 equiv) tert-butyl carbamate (160 mg, 1.368 mmol, 2 equiv), XantPhos (40 mg, 0.068 mmol, 0.1 equiv) Pd₂(dba)₃ (63 mg, 0.068 mmol, 0.1 equiv) at 90° C. and stirred for overnight. After completion of reaction, the reaction mixture was concentrated under reduced pressure to give crude product which was further purified by column chromatography (PE/EA=2:1) afford desired compound tert-butyl (5-(3-chlorobenzyl)-6-oxo-5,6,7,8-tetrahydro-1,5-naphthyridin-2-yl)carbamate (204 mg, 76.93%) as a yellow semi-solid. MS (ESI): mass calcd. for C₂₀H₂₂ClN₃O₃, 387.13 m/z, found 388.20 [M+H]⁺.

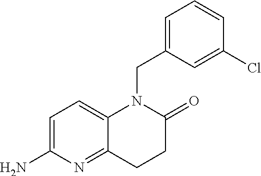 [SMILES: Nc1ccc2c(n1)CCC(=O)N2Cc1cccc(Cl)c1]

### 6-amino-1-(3-chlorobenzyl)-3,4-dihydro-1,5-naphthyridin-2(1H)-one

To a stirred solution of tert-butyl (5-(3-chlorobenzyl)-6-oxo-5,6,7,8-tetrahydro-1,5-naphthyridin-2-yl)carbamate (220 mg, 0.567 mmol, 1 equiv) in EA (5 mL), HCl (4N, in EA, 10 mL) was added at rt. The reaction stirred for 2 h. The resulting mixture was concentrated under reduced pressure to afford 6-amino-1-(3-chlorobenzyl)-3,4-dihydro-1,5-naphthyridin-2(1H)-one (250 mg, 153.17%) as a white solid. MS (ESI): mass calcd. for C₁₅H₁₄ClN₃O, 287.08 m/z, found 288.20 [M+H]⁺.

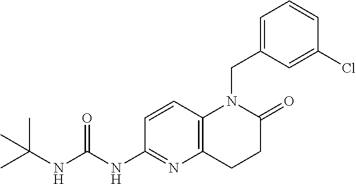 [SMILES: CC(C)(C)NC(=O)Nc1ccc2c(n1)CCC(=O)N2Cc1cccc(Cl)c1]

### 1-(tert-butyl)-3-(5-(3-chlorobenzyl)-6-oxo-5,6,7,8-tetrahydro-1,5-naphthyridin-2-yl)urea

A solution of 6-amino-1-(3-chlorobenzyl)-3,4-dihydro-1,5-naphthyridin-2(1H)-one (100 mg, 0.348 mmol, 1 equiv), TEA (176 mg, 1.740 mmol, 5 equiv) and 2-isocyanato-2-methylpropane (172 mg, 1.740 mmol, 5 equiv) in DCM (10 mL) was stirred for overnight at rt. The reaction was concentrated under vacuum. The residue was purified by silica gel column chromatography, eluted with EA (55%-62%) to afford crude products. The residue was purified by reversed-phase flash chromatography with the following conditions Column: Xselect CSH C18 OBD Column 30*150 mm 5 μm, n; Mobile Phase A: Water (0.1% FA), Mobile Phase B: ACN; Flow rate: 60 mL/min; Gradient: 35% B to 55% B in 10 min; Wave Length: 254/220 nm; RT1(min): 9.8; Number Of Runs: 3 to) afford 1-(tert-butyl)-3-(5-(3-chlorobenzyl)-6-oxo-5,6,7,8-tetrahydro-1,5-naphthyridin-2-yl)urea (26.2 mg, 19.44%) as a white solid. MS (ESI): mass calcd. for C₂₀H₂₃ClN₄O₂: 386.15 m/z, found 387.10 [M+H]⁺. ¹H NMR (300 MHz, DMSO-d₆) δ 8.81 (s, 1H), 7.94 (s, 1H), 7.11-7.36 (m, 6H), 5.08 (s, 2H), 2.97 (dd, J=8.8, 6.2 Hz, 2H), 2.78 (dd, J=8.7, 6.2 Hz, 2H), 1.30 (s, 9H).

### Example 282: (S)-1-(tert-butyl)-3-(6-oxo-5-(1-(3-(trifluoromethyl)phenyl)ethyl)-5,6,7,8-tetrahydro-1,5-naphthyridin-2-yl)urea

### Synthetic Scheme

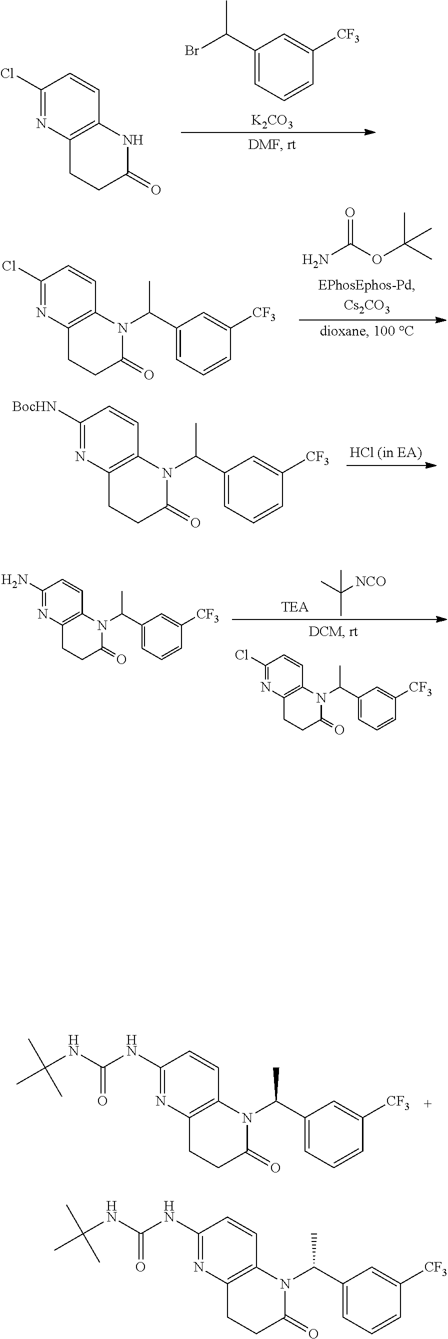

### 6-chloro-1-(1-(3-(trifluoromethyl)phenyl)ethyl)-3,4-dihydro-1,5-naphthyridin-2(1H)-one

To a solution of 6-chloro-3,4-dihydro-1H-1,5-naphthyridin-2-one (400 mg, 2.190 mmol, 1 equiv) in DMF (10 mL) was added K₂CO₃ (908 mg, 6.571 mmol, 3 equiv) and 1-(1-bromoethyl)-3-(trifluoromethyl)benzene (831 mg, 3.286 mmol, 1 equiv). The resulting mixture was placed at rt and stirred overnight until the starting material was totally consumed by LCMS. The mixture was diluted with EtOAc (50 mL), washed with water (3×30 mL), brine (2×30 ML) and concentrated under vacuum to yield a crude product which was directly purified by flash chromatography (0-100% ethyl acetate/petroleum ether) to afford 6-chloro-1-(1-(3-(trifluoromethyl)phenyl)ethyl)-3,4-dihydro-1,5-naphthyridin-2(1H)-one (300 mg, 38.29%) as a yellow solid. MS (ESI): mass calcd. for C₁₇H₁₄ClF₃N₂O 354.07 m/z, found 355.05 [M+H]f.

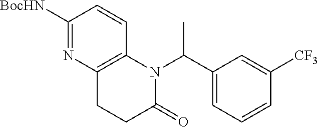 [SMILES: CC(c1cccc(C(F)(F)F)c1)N1C(=O)CCc2nc(NC(=O)OC(C)(C)C)ccc21]

### tert-butyl (6-oxo-5-(1-(3-(trifluoromethyl)phenyl)ethyl)-5,6,7,8-tetrahydro-1,5-naphthyridin-2-yl)carbamate

To a solution of 6-chloro-1-(1-(3-(trifluoromethyl)phenyl)ethyl)-3,4-dihydro-1,5-naphthyridin-2(1H)-one (300 mg, 0.846 mmol, 1 equiv) in dioxane (10 mL) was added tert-butyl carbamate (198.1 mg, 1.691 mmol, 2 equiv), Ephos (45.2 mg, 0.085 mmol, 0.1 equiv), EPhos Pd G4 (77.6 mg, 0.085 mmol, 0.1 equiv) and Cs₂CO₃ (826.5 mg, 2.537 mmol, 3 equiv). The resulting mixture was placed at 100° C. under a nitrogen atmosphere and stirred overnight until the starting material was totally consumed by LCMS, The reaction mixture was quenched by water and extracted with EA (3×20 mL). The combined organic extracts were washed with brine (20 mL), dried over anhydrous Na₂SO₄, and concentrated under vacuum to yield a crude product which was directly purified by flash chromatography (0-100% ethyl acetate/petroleum ether) to afford tert-butyl (6-oxo-5-(1-(3-(trifluoromethyl)phenyl)ethyl)-5,6,7,8-tetrahydro-1,5-naphthyridin-2-yl)carbamate (200 mg, 54.31%) as a yellow solid. MS (ESI): mass calcd. for C₂₂H₂₄F₃N₃O₃, 435.18 m/z, found 436.15 [M+H]⁺.

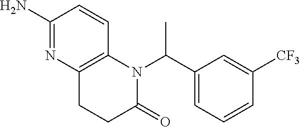 [SMILES: CC(c1cccc(C(F)(F)F)c1)N1C(=O)CCc2nc(N)ccc21]

### 6-amino-1-(1-(3-(trifluoromethyl)phenyl)ethyl)-3,4-dihydro-1,5-naphthyridin-2(1H)-one

To a solution of tert-butyl (6-oxo-5-(1-(3-(trifluoromethyl)phenyl)ethyl)-5,6,7,8-tetrahydro-1,5-naphthyridin-2-yl)carbamate (20 mg, 0.046 mmol, 1 equiv) in HCl (4N, in EA, 2 mL). The resulting mixture was placed at rt and stirred overnight until the starting material was totally consumed by LCMS. The reaction mixture was concentrated under vacuum to yield 6-amino-1-{1-[3-(trifluoromethyl)phenyl]ethyl}-3,4-dihydro-1,5-naphthyridin-2-one (100 mg, 61.75%) as a yellow solid. MS (ESI): mass calcd. for C₁₇H₁₆F₃N₃O, 335.12 m/z, found 336.05 [M+H]⁺.

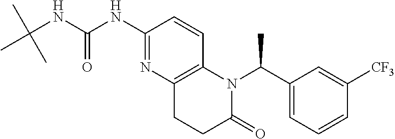 [SMILES: C[C@@H](c1cccc(C(F)(F)F)c1)N1C(=O)CCc2nc(NC(=O)NC(C)(C)C)ccc21]

### (S)-1-(tert-butyl)-3-(6-oxo-5-(1-(3-(trifluoromethyl)phenyl)ethyl)-5,6,7,8-tetrahydro-1,5-naphthyridin-2-yl)urea

To a stirred solution of 6-amino-1-(1-(3-(trifluoromethyl)phenyl)ethyl)-3,4-dihydro-1,5-naphthyridin-2(1H)-one (100 mg, 0.298 mmol, 1 equiv) in DCM (10.00 mL) was added 2-isocyanato-2-methylpropane (147.8 mg, 1.491 mmol, 5 equiv) and TEA (90.5 mg, 0.895 mmol, 3 equiv) stirred overnight at rt. The reaction progress was monitored by LCMS. The reaction mixture was quenched by water (10 mL) and extracted with EA (3×20 mL). The combined organic extracts were washed with brine (20 mL), dried over anhydrous Na₂SO₄, and concentrated under vacuum to yield a crude product which was directly purified by flash chromatography (0-100% ethyl acetate/petroleum ether) to afford 100 mg crude product as a white solid. The compound was separated by Chiral-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 45% B to 73% B in 9 min, 73% B; Wave Length: 254/220 nm; RT1(min): 9.6; Number Of Runs: 0) to afford (S)-1-(tert-butyl)-3-(6-oxo-5-(1-(3-(trifluoromethyl)phenyl)ethyl)-5,6,7,8-tetrahydro-1,5-naphthyridin-2-yl)urea (11.7 mg, 8.86%) as a white solid. MS (ESI): mass calcd. for C₂₂H₂₅F₃N₄O₂, 434.19 m/z, found 435.10 [M+H]⁺. ¹H NMR (300 MHz, DMSO-d₆) δ 8.89 (s, 1H), 7.96 (s, 1H), 7.52-7.67 (m, 4H), 6.96-7.07 (m, 2H), 6.08 (d, J=7.3 Hz, 1H), 2.61-3.06 (m, 4H), 1.74 (d, J=7.1 Hz, 3H), 1.30 (s, 9H). And another isomer (R)-1-(tert-butyl)-3-(6-oxo-5-(1-(3-(trifluoromethyl)phenyl)ethyl)-5,6,7,8-tetrahydro-1,5-naphthyridin-2-yl)urea (9.1 mg, 6.96%) as a white solid. MS (ESI): mass calcd. for C₂₂H₂₅F₃N₄O₂, 434.19 m/z, found 435.10 [M+H]⁺. ¹H NMR (300 MHz, DMSO-d₆) δ 8.89 (s, 1H), 7.96 (s, 1H), 7.52-7.67 (m, 4H), 6.96-7.07 (m, 2H), 6.08 (d, J=7.3 Hz, 1H), 2.61-3.06 (m, 4H), 1.74 (d, J=7.1 Hz, 3H), 1.30 (s, 9H).

### Example 283: (R)-1-(tert-butyl)-3-(6-oxo-5-(1-(3-(trifluoromethyl)phenyl)ethyl)-5,6,7,8-tetrahydro-1,5-naphthyridin-2-yl)urea

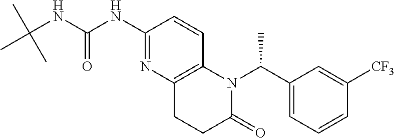 [SMILES: C[C@H](c1cccc(C(F)(F)F)c1)N1C(=O)CCc2nc(NC(=O)NC(C)(C)C)ccc21]

### (R)-1-(tert-butyl)-3-(6-oxo-5-(1-(3-(trifluoromethyl)phenyl)ethyl)-5,6,7,8-tetrahydro-1,5-naphthyridin-2-yl)urea

To a stirred solution of 6-amino-1-(1-(3-(trifluoromethyl)phenyl)ethyl)-3,4-dihydro-1,5-naphthyridin-2(1H)-one (100 mg, 0.298 mmol, 1 equiv) in DCM (10.00 mL) was added 2-isocyanato-2-methylpropane (147.8 mg, 1.491 mmol, 5 equiv) and TEA (90.5 mg, 0.895 mmol, 3 equiv) stirred overnight at rt. The reaction progress was monitored by LCMS. The reaction mixture was quenched by water (10 mL) and extracted with EA (3×20 mL). The combined organic extracts were washed with brine (20 mL), dried over anhydrous Na₂SO₄, and concentrated under vacuum to yield a crude product which was directly purified by flash chromatography (0-100% ethyl acetate/petroleum ether) to afford 100 mg crude product as a white solid. The compound was separated by Chiral-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 45% B to 73% B in 9 min, 73% B; Wave Length: 254/220 nm; RT1(min): 9.6; Number Of Runs: 0) to afford (S)-1-(tert-butyl)-3-(6-oxo-5-(1-(3-(trifluoromethyl)phenyl)ethyl)-5,6,7,8-tetrahydro-1,5-naphthyridin-2-yl)urea (11.7 mg, 8.86%) as a white solid. MS (ESI): mass calcd. for C₂₂H₂₅F₃N₄O₂, 434.19 m/z, found 435.10 [M+H]⁺. ¹H NMR (300 MHz, DMSO-d₆) δ 8.89 (s, 1H), 7.96 (s, 1H), 7.52-7.67 (m, 4H), 6.96-7.07 (m, 2H), 6.08 (d, J=7.3 Hz, 1H), 2.61-3.06 (m, 4H), 1.74 (d, J=7.1 Hz, 3H), 1.30 (s, 9H). And another isomer (R)-1-(tert-butyl)-3-(6-oxo-5-(1-(3-(trifluoromethyl)phenyl)ethyl)-5,6,7,8-tetrahydro-1,5-naphthyridin-2-yl)urea (9.1 mg, 6.96%) as a white solid. MS (ESI): mass calcd. for C₂₂H₂₅F₃N₄O₂, 434.19 m/z, found 435.10 [M+H]⁺. ¹H NMR (300 MHz, DMSO-d₆) δ 8.89 (s, 1H), 7.96 (s, 1H), 7.52-7.67 (m, 4H), 6.96-7.07 (m, 2H), 6.08 (d, J=7.3 Hz, 1H), 2.61-3.06 (m, 4H), 1.74 (d, J=7.1 Hz, 3H), 1.30 (s, 9H).

### Example 284: 3-(1-benzyl-2-oxo-3,4-dihydroquinolin-6-yl)-1-phenylurea

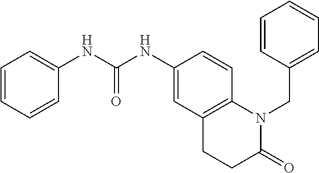 [SMILES: O=C(Nc1ccccc1)Nc1ccc2c(c1)CCC(=O)N2Cc1ccccc1]

### 3-(1-benzyl-2-oxo-3,4-dihydroquinolin-6-yl)-1-phenylurea

To a stirred solution of 6-amino-1-benzyl-3,4-dihydroquinolin-2-one (150 mg, 0.594 mmol, 1 equiv) in DCM (2 mL) was added triphosgene (59.98 mg, 0.202 mmol, 0.34 equiv) in portions at 0° C. under nitrogen atmosphere. The resulting mixture was stirred for 30 min at 0° C. under nitrogen atmosphere. To the above mixture was added triethylamine (180.47 mg, 1.782 mmol, 3 equiv) in portions at room temperature. The resulting mixture was stirred for 30 min at room temperature under nitrogen atmosphere. To the above mixture was added aniline (55.36 mg, 0.594 mmol, 1 equiv) in portions at 0° C. The resulting mixture was stirred for 30 min at room temperature under nitrogen atmosphere. The reaction was monitored by LCMS. Quenched with water (10 mL) and extracted with DCM (3×10 mL). The combined extracts were washed with water, brine, dried over Na₂SO₄, filtered and concentrated. The crude product was purified by Prep-HPLC using a X Bridge Prep OBD C18 150 mm×30 mm×5 μm column (eluent: 26% to 45% (v/v) CH₃CN and H₂O with 10 mmol/L NH₄HCO₃) to afford 3-(1-benzyl-2-oxo-3,4-dihydroquinolin-6-yl)-1-phenylurea (37.5 mg, 16.92%) as a white solid. LC/MS (ESI): mass calcd. for C₂₃H₂₁N₃O₂:371.2 m/z, found: 372.10 [M+H]⁺. ¹H NMR (300 MHz, DMSO-d₆) δ 8.63 (s, 1H), 8.55 (s, 1H), 7.43-7.41 (m, 3H), 7.31-7.21 (m, 8H), 7.01-6.89 (m, 1H), 6.84 (d, J=8.7 Hz, 1H), 5.12 (s, 2H), 2.93 (m, 2H), 2.69 (m, 2H).

### Example 285: N-(1-benzyl-2-oxo-3,4-dihydroquinolin-6-yl)-3,3-dimethylbutanamide

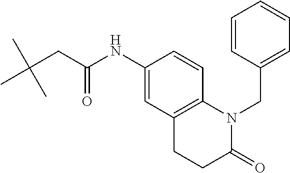 [SMILES: CC(C)(C)CC(=O)Nc1ccc2c(c1)CCC(=O)N2Cc1ccccc1]

### N-(1-benzyl-2-oxo-3,4-dihydroquinolin-6-yl)-3,3-dimethylbutanamide

To a stirred solution of 3,3-dimethylbutanamide (35.15 mg, 0.305 mmol, 1.1 equiv) and HATU (137.13 mg, 0.360 mmol, 1.3 equiv) in DMF (1 mL) were added 6-amino-1-benzyl-3,4-dihydroquinolin-2-one (70 mg, 0.277 mmol, 1 equiv) and DIEA (107.57 mg, 0.831 mmol, 3 equiv) in portions at room temperature under nitrogen atmosphere. The resulting mixture was stirred for 2 h at room temperature under nitrogen atmosphere. The reaction was monitored by LCMS. Quenched with water (10 mL) and extracted with EA (3×10 mL). The combined extracts were washed with water, brine, dried over Na₂SO₄, filtered and concentrated. The crude product was purified by Prep-HPLC using a X Bridge Prep OBD C18 150 mm×30 mm×5 μm column (eluent: 26% to 42% (v/v) CH₃CN and H₂O with 10 mmol/L NH₄HCO₃) to afford N-(1-benzyl-2-oxo-3,4-dihydroquinolin-6-yl)-3,3-dimethylbutanamide (47.6 mg, 48.80%) as a white solid. LC/MS (ESI): mass calcd. for C₂₂H₂₆N₂O₂:350.2 m/z, found: 351.15 [M+H]⁺. ¹H NMR (300 MHz, DMSO-d₆) δ 9.69 (s, 1H), 7.51 (d, J=2.1 Hz, 1H), 7.33-7.19 (m, 6H), 6.84 (d, J=8.7 Hz, 1H), 5.11 (s, 2H), 2.92-2.88 (m, 2H), 2.69-2.64 (m, 2H), 2.13 (s, 2H), 0.99 (s, 9H).

### Example 286: (S)-1-(tert-butyl)-3-(1-(3-chlorobenzyl)-3-methyl-2-oxo-2,3-dihydro-1H-pyrido[2,3-b][1,4]oxazin-6-yl)urea

### Synthetic Scheme

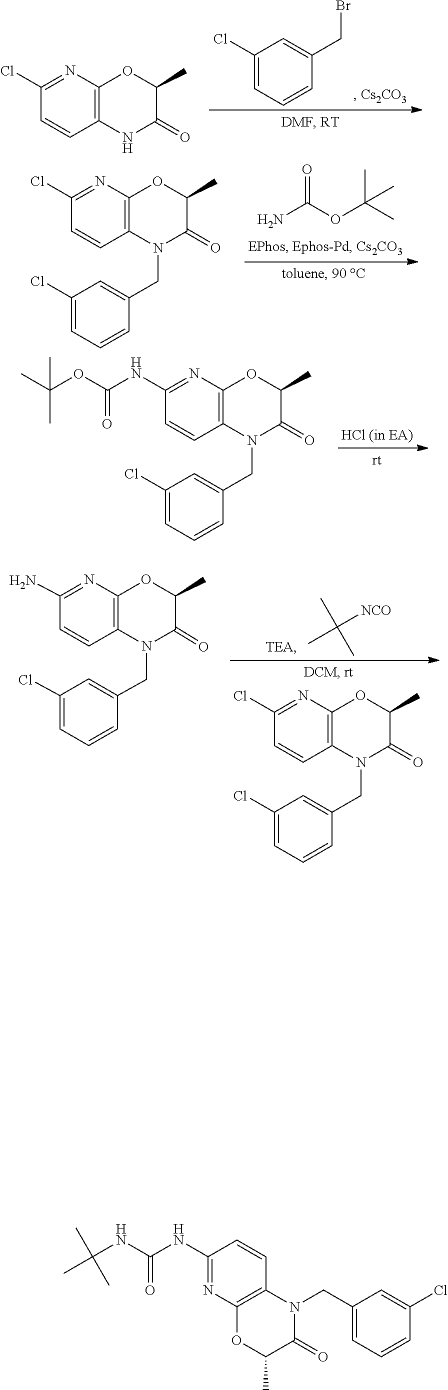

### (S)-6-chloro-1-(3-chlorobenzyl)-3-methyl-1H-pyrido[2,3-b][1,4]oxazin-2(3H)-one

A solution of (S)-6-chloro-3-methyl-1H-pyrido[2,3-b][1,4]oxazin-2(3H)-one (100 mg, 0.503 mmol, 1 equiv) in DMF (5 mL) was added 1-(bromomethyl)-3-chlorobenzene (155 mg, 0.754 mmol, 1.5 equiv) and Cs₂CO₃ (492 mg, 1.509 mmol, 3.0 equiv) at rt. The resulting mixture was stirred for 2 h at rt. The reaction mixture was quenched by ice water (5 mL) and extracted with EA (3×5 mL). The organic layers were combined, dried over anhydrous sodium sulfate, filtered and concentrated under reduced pressure. The residue was purified by silica gel column, eluted with PE/EA (3:1) to afford (S)-6-chloro-1-(3-chlorobenzyl)-3-methyl-1H-pyrido[2,3-b][1,4]oxazin-2(3H)-one (150 mg, 92.19%) as a white solid. MS (ESI): mass calcd. for C₁₅H₁₂Cl₂N₂O₂, 322.03 m/z, found 323.00 [M+H]⁺.

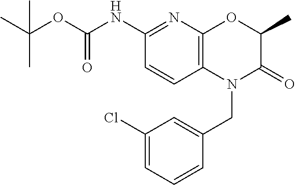 [SMILES: C[C@@H]1Oc2nc(NC(=O)OC(C)(C)C)ccc2N(Cc2cccc(Cl)c2)C1=O]

### tert-butyl (S)-(1-(3-chlorobenzyl)-3-methyl-2-oxo-2,3-dihydro-1H-pyrido[2,3-b][1,4]oxazin-6-yl)carbamate

A solution of (S)-6-chloro-1-(3-chlorobenzyl)-3-methyl-1H-pyrido[2,3-b][1,4]oxazin-2(3H)-one (150 mg, 0.464 mmol, 1 equiv) in toluene (5 mL) was added tert-butyl carbamate (70 mg, 0.603 mmol, 1.3 equiv), Cs₂CO₃ (453 mg, 1.392 mmol, 3.0 equiv), Ephos (49 mg, 0.093 mmol, 0.2 equiv) and EPhos Pd G4 (42 mg, 0.046 mmol, 0.1 equiv) at rt. The resulting mixture was stirred for overnight at 90° C. under N₂. After cooled to room temperature the reaction mixture was quenched by water (5 mL) and extracted with EA (3×5 mL). The organic layers were combined, dried over anhydrous sodium sulfate, filtered and concentrated under reduced pressure. The residue was purified by silica gel column, eluted with PE/EA (3:1) to afford tert-butyl (S)-(1-(3-chlorobenzyl)-3-methyl-2-oxo-2,3-dihydro-1H-pyrido[2,3-b][1,4]oxazin-6-yl)carbamate (160 mg, 85.35%) as yellow oil. MS (ESI): mass calcd. for C₂₀H₂₂ClN₃O₄, 403.13 m/z, found 404.20 [M+H]⁺.

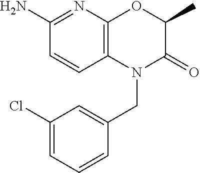 [SMILES: C[C@@H]1Oc2nc(N)ccc2N(Cc2cccc(Cl)c2)C1=O]

### (S)-6-amino-1-(3-chlorobenzyl)-3-methyl-1H-pyrido[2,3-b][1,4]oxazin-2(3H)-one

A solution of tert-butyl (S)-(1-(3-chlorobenzyl)-3-methyl-2-oxo-2,3-dihydro-1H-pyrido[2,3-b][1,4]oxazin-6-yl)carbamate (150 mg, 0.371 mmol, 1 equiv) in EA (5 mL), 2N HCl (in EA, 5 mL) was added into the reaction at rt. The resulting mixture was stirred for 2 h at rt. The resulting mixture was concentrated under reduced pressure. The crude product (S)-6-amino-1-(3-chlorobenzyl)-3-methyl-1H-pyrido[2,3-b][1,4]oxazin-2(3H)-one (80 mg, 70.91%) was used in the next step directly without further purification. MS (ESI): mass calcd. for C₁₅H₁₄ClN₃O₂, 303.08 m/z, found 304.15 [M+H]⁺.

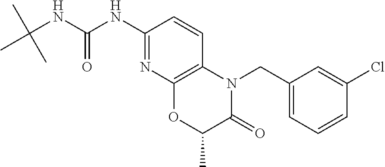 [SMILES: C[C@@H]1Oc2nc(NC(=O)NC(C)(C)C)ccc2N(Cc2cccc(Cl)c2)C1=O]

### (S)-1-(tert-butyl)-3-(1-(3-chlorobenzyl)-3-methyl-2-oxo-2,3-dihydro-1H-pyrido[2,3-b][1,4]oxazin-6-yl)urea

A solution of (S)-6-amino-1-(3-chlorobenzyl)-3-methyl-1H-pyrido[2,3-b][1,4]oxazin-2(3H)-one (80 mg, 0.263 mmol, 1 equiv) in DCM (5 mL) was added 2-isocyanato-2-methylpropane (261 mg, 2.630 mmol, 10 equiv) and TEA (266 mg, 2.630 mmol, 10 equiv) at rt. The resulting mixture was stirred for overnight at rt. The reaction mixture was quenched by water (10 mL) and extracted with DCM (3×10 mL). The organic layers were combined, dried over anhydrous sodium sulfate, filtered and concentrated under vacuum. The crude was purified by flash chromatography with PE/EA (1:1) and Prep-HPLC (Column: XBridge Prep OBD C18 Column, 30*150 mm, 5 μm; Flow rate: 60 mL/min; Gradient: 30% B to 60% B in 7 min, 60% B; Wave Length: 254/220 nm; RT1(min): 6.7; Number Of Runs: 0) to afford (S)-1-(tert-butyl)-3-(1-(3-chlorobenzyl)-3-methyl-2-oxo-2,3-dihydro-1H-pyrido[2,3-b][1,4]oxazin-6-yl)urea (19.6 mg, 17.86%) as a white solid. MS (ESI): mass calcd. C₂₀H₂₃ClN₄O₃ for: 402.15 m/z, found 403.05 [M+H]⁺. ¹H NMR (400 MHz, DMSO-d₆) δ 8.73 (s, 1H), 7.29-7.41 (m, 4H), 7.17-7.26 (m, 2H), 7.09 (s, 1H), 5.02-5.16 (m, 3H), 1.53 (d, J=6.8 Hz, 3H), 1.28 (s, 9H).

### Example 287: (R)-1-(tert-butyl)-3-(1-(3-chlorobenzyl)-3-methyl-2-oxo-2,3-dihydro-1H-pyrido[2,3-b][1,4]oxazin-6-yl)urea

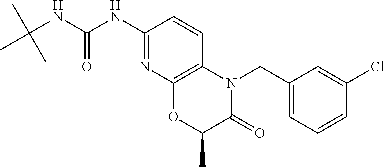 [SMILES: C[C@H]1Oc2nc(NC(=O)NC(C)(C)C)ccc2N(Cc2cccc(Cl)c2)C1=O]

### (R)-1-(tert-butyl)-3-(1-(3-chlorobenzyl)-3-methyl-2-oxo-2,3-dihydro-1H-pyrido[2,3-b][1,4]oxazin-6-yl)urea

A solution of (R)-6-amino-1-(3-chlorobenzyl)-3-methyl-1H-pyrido[2,3-b][1,4]oxazin-2(3H)-one (60 mg, 0.198 mmol, 1 equiv) in DCM (5 mL) was added 2-isocyanato-2-methylpropane (195 mg, 1.980 mmol, 10 equiv) and TEA (199 mg, 1.980 mmol, 10 equiv) at rt. The resulting mixture was stirred for overnight at rt. The reaction mixture was quenched by water (10 mL) and extracted with DCM (3×10 mL). The organic layers were combined, dried over anhydrous sodium sulfate, filtered and concentrated under vacuum. The crude was purified by flash chromatography with PE/EA (1:1) and Prep-HPLC (Column: XBridge Prep OBD C18 Column, 30*150 mm, 5 μm; Flow rate: 60 mL/min; Gradient: 30% B to 60% B in 7 min, 60% B; Wave Length: 254/220 nm; RT1(min): 6.7; Number Of Runs: 0) to afford (R)-1-(tert-butyl)-3-(1-(3-chlorobenzyl)-3-methyl-2-oxo-2,3-dihydro-1H-pyrido[2,3-b][1,4]oxazin-6-yl)urea (12.1 mg, 14.76%) as a white solid. MS (ESI): mass calcd. C₂₀H₂₃ClN₄O₃ for: 402.15 m/z, found 403.05[M+H]⁺. ¹H NMR (400 MHz, DMSO-d₆) δ 8.73 (s, 1H), 7.29-7.41 (m, 4H), 7.17-7.26 (m, 2H), 7.09 (s, 1H), 5.02-5.16 (m, 3H), 1.53 (d, J=6.8 Hz, 3H), 1.28 (s, 9H).

### Example 288: (S)-1-(4-benzyl-2-(methoxymethyl)-3-oxo-3,4-dihydro-2H-benzo[b][1,4]oxazin-7-yl)-3-(tert-butyl)urea

### Synthetic Scheme

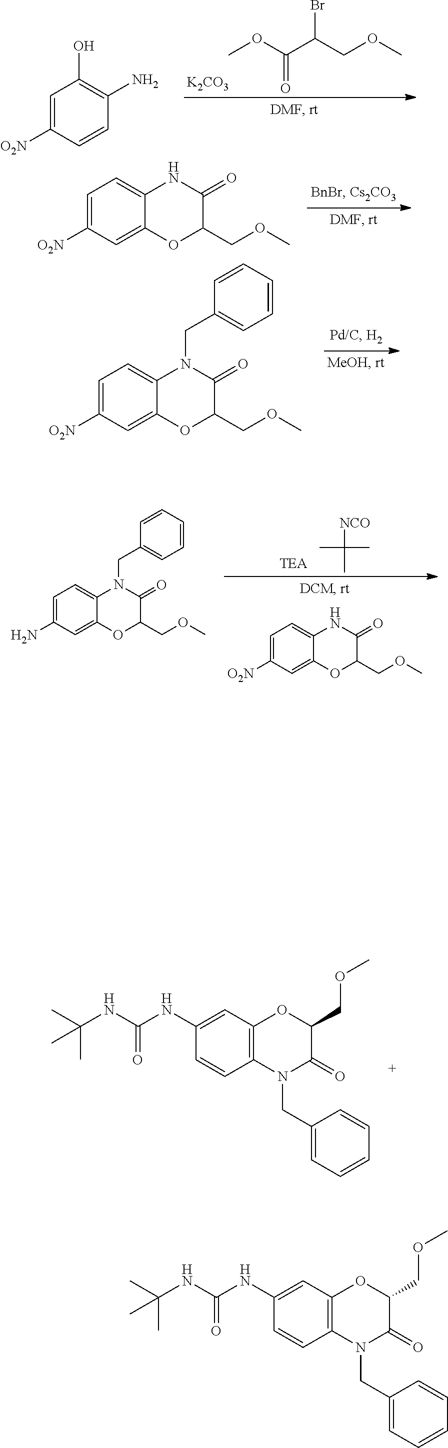

### 2-(methoxymethyl)-7-nitro-2H-benzo[b][1,4]oxazin-3(4H)-one

A solution of 2-amino-5-nitrophenol (300 mg, 1.946 mmol, 1 equiv) in dimethylformamide (5 mL) was added Cs₂CO₃ (1.9 g, 5.838 mmol, 3.0 equiv) and methyl 2-bromo-3-methoxypropanoate (460.2 mg, 2.335 mmol, 1.2 equiv) at rt. The resulting mixture was stirred for overnight at 90° C. The reaction mixture was quenched by water (5 mL) and extracted with EA (3×10 mL). The combined organic layers were washed with NaCl (20 mL), dried over anhydrous Na₂SO₄. After filtration, the filtrate was concentrated under reduced pressure. The residue was purified by silica gel column chromatography, eluted with PE/EA (3/1) to afford 2-(methoxymethyl)-7-nitro-2H-benzo[b][1,4]oxazin-3(4H)-one (200 mg, 43.14%) as a white solid. MS (ESI): mass calcd. for C₁₀H₁₀N₂O₅ 238.06 m/z, found 239.10 [M+H]⁺.

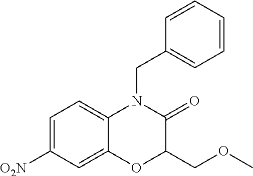 [SMILES: COCC1Oc2cc([N+](=O)[O-])ccc2N(Cc2ccccc2)C1=O]

### 4-benzyl-2-(methoxymethyl)-7-nitro-2H-benzo[b][1,4]oxazin-3(4H)-one

A solution of 2-(methoxymethyl)-7-nitro-2H-benzo[b][1,4]oxazin-3(4H)-one (150 mg, 0.630 mmol, 1 equiv) in dimethylformamide (5 mL) was added BnBr (323.1 mg, 1.890 mmol, 3.0 equiv) and Cs₂CO₃ (615.5 mg, 1.890 mmol, 3.0 equiv) at rt. The resulting mixture was stirred for 2 h at rt. The reaction mixture was quenched by water (10 mL) and extracted with EA (3×10 mL). The combined organic layers were washed with NaCl (10 mL), dried over anhydrous Na₂SO₄. After filtration, the filtrate was concentrated under reduced pressure. The residue was purified by silica gel column chromatography, eluted with PE/EA (3/1) to afford 4-benzyl-2-(methoxymethyl)-7-nitro-2H-benzo[b][1,4]oxazin-3(4H)-one (130 mg, 62.88%) as a white solid. MS (ESI): mass calcd. for C₁₇H₁₆N₂O₅, 328.11 m/z, found 329.05 [M+H]⁺.

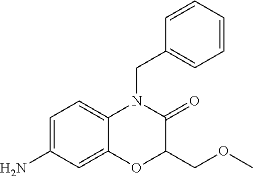 [SMILES: COCC1Oc2cc(N)ccc2N(Cc2ccccc2)C1=O]

### 7-amino-4-benzyl-2-(methoxymethyl)-2H-benzo[b][1,4]oxazin-3(4H)-one

To a solution of 4-benzyl-2-(methoxymethyl)-7-nitro-2H-benzo[b][1,4]oxazin-3(4H)-one (130 mg, 0.396 mmol, 1 equiv) in methanol (5 mL) was added Pd/C (10%, 421.3 mg, 3.960 mmol, 10 equiv) under nitrogen atmosphere. The mixture was hydrogenated at room temperature for 30 min under hydrogen atmosphere using a hydrogen balloon, filtered through a Celite pad and concentrated under reduced pressure. The crude product 7-amino-4-benzyl-2-(methoxymethyl)-2H-benzo[b][1,4]oxazin-3(4H)-one (100 mg, 84.65%) was used in the next step directly without further purification. MS (ESI): mass calcd. for C₁₇H₁₈N₂O₃, 298.13 m/z, found 353.10 [M+H]⁺.

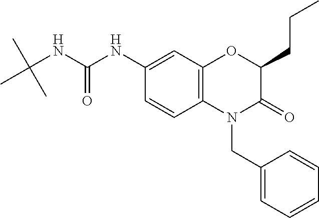 [SMILES: CCC[C@@H]1Oc2cc(NC(=O)NC(C)(C)C)ccc2N(Cc2ccccc2)C1=O]

### (S)-1-(4-benzyl-2-(methoxymethyl)-3-oxo-3,4-dihydro-2H-benzo[b][1,4]oxazin-7-yl)-3-(tert-butyl)urea

A solution of 7-amino-4-benzyl-2-(methoxymethyl)-2H-benzo[b][1,4]oxazin-3(4H)-one (70 mg, 0.235 mmol, 1 equiv) in DCM (5 mL) was added 2-isocyanato-2-methylpropane (232.6 mg, 2.350 mmol, 10 equiv) and Et₃N (237.4 mg, 2.350 mmol, 10 equiv) at rt. The resulting mixture was stirred for overnight at rt. The reaction mixture was quenched by water (10 mL) and extracted with EA (3×20 mL). The combined organic layers were washed with NaCl (20 mL), dried over anhydrous Na₂SO₄. After filtration, the filtrate was concentrated under reduced pressure. The crude product 40 mg was purified by Prep-Chiral-HPLC with the following conditions (Column: CHIRALPAK IF, 2*25 cm, 5 μm; Mobile Phase A: Hex(0.5% 2M NH₃-MeOH)-HPLC, Mobile Phase B: EtOH-HPLC; Flow rate: 20 mL/min; Gradient: 25% B to 25% B in 23 min; Wave Length: 220/254 nm; RT1(min): 13.384; RT2(min): 18.251; Sample Solvent: EtOH-HPLC; Injection Volume: 0.6 mL; Number Of Runs: 7) to afford (S)-1-(4-benzyl-2-(methoxymethyl)-3-oxo-3,4-dihydro-2H-benzo[b][1,4]oxazin-7-yl)-3-(tert-butyl)urea (20.0 mg, 21.32%) as a white solid. MS (ESI): mass calcd. C₂₂H₂₇N₃O₄ for: 397.20 m/z, found 398.15 [M+H]⁺. ¹H NMR (400 MHz, DMSO-d₆) δ 8.19 (s, 1H), 7.29-7.42 (m, 2H), 7.19-7.29 (m, 4H), 6.71-6.88 (m, 1H), 6.57-6.71 (m, 1H), 5.94 (s, 1H), 5.10-5.31 (m, 1H), 4.89-5.10 (m, 2H), 3.71-3.81 (m, 2H), 3.30 (s, 3H), 1.25 (s, 9H). And to afford (R)-1-(4-benzyl-2-(methoxymethyl)-3-oxo-3,4-dihydro-2H-benzo[b][1,4]oxazin-7-yl)-3-(tert-butyl)urea (20.5 mg, 21.91%) as a white solid. MS (ESI): mass calcd. C₂₂H₂₇N₃O₄ for: 397.20 m/z, found 398.15[M+H]⁺. ¹H NMR (400 MHz, DMSO-d₆) δ 8.19 (s, 1H), 7.29-7.42 (m, 2H), 7.19-7.29 (m, 4H), 6.71-6.88 (m, 1H), 6.57-6.71 (m, 1H), 5.94 (s, 1H), 5.10-5.31 (m, 1H), 4.89-5.10 (m, 2H), 3.71-3.81 (m, 2H), 3.30 (s, 3H), 1.25 (s, 9H).

### Example 289: (R)-1-(4-benzyl-2-(methoxymethyl)-3-oxo-3,4-dihydro-2H-benzo[b][1,4]oxazin-7-yl)-3-(tert-butyl)urea

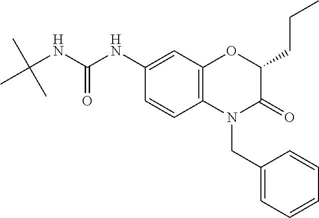 [SMILES: CCC[C@H]1Oc2cc(NC(=O)NC(C)(C)C)ccc2N(Cc2ccccc2)C1=O]

### (R)-1-(4-benzyl-2-(methoxymethyl)-3-oxo-3,4-dihydro-2H-benzo[b][1,4]oxazin-7-yl)-3-(tert-butyl)urea

A solution of 7-amino-4-benzyl-2-(methoxymethyl)-2H-benzo[b][1,4]oxazin-3(4H)-one (70 mg, 0.235 mmol, 1 equiv) in DCM (5 mL) was added 2-isocyanato-2-methylpropane (232.6 mg, 2.350 mmol, 10 equiv) and Et₃N (237.4 mg, 2.350 mmol, 10 equiv) at rt. The resulting mixture was stirred for overnight at rt. The reaction mixture was quenched by water (10 mL) and extracted with EA (3×20 mL). The combined organic layers were washed with NaCl (20 mL), dried over anhydrous Na₂SO₄. After filtration, the filtrate was concentrated under reduced pressure. The crude product 40 mg was purified by Prep-Chiral-HPLC with the following conditions (Column: CHIRALPAK IF, 2*25 cm, 5 μm; Mobile Phase A: Hex(0.5% 2M NH₃-MeOH)—HPLC, Mobile Phase B: EtOH—HPLC; Flow rate: 20 mL/min; Gradient: 25% B to 25% B in 23 min; Wave Length: 220/254 nm; RT1(min): 13.384; RT2(min): 18.251; Sample Solvent: EtOH—HPLC; Injection Volume: 0.6 mL; Number Of Runs: 7) to afford (S)-1-(4-benzyl-2-(methoxymethyl)-3-oxo-3,4-dihydro-2H-benzo[b][1,4]oxazin-7-yl)-3-(tert-butyl)urea (20.0 mg, 21.32%) as a white solid. MS (ESI): mass calcd. C₂₂H₂₇N₃O₄ for: 397.20 m/z, found 398.15 [M+H]⁺. 1H NMR (400 MHz, DMSO-d₆) δ 8.19 (s, 1H), 7.29-7.42 (m, 2H), 7.19-7.29 (m, 4H), 6.71-6.88 (m, 1H), 6.57-6.71 (m, 1H), 5.94 (s, 1H), 5.10-5.31 (m, 1H), 4.89-5.10 (m, 2H), 3.71-3.81 (m, 2H), 3.30 (s, 3H), 1.25 (s, 9H). And to afford (R)-1-(4-benzyl-2-(methoxymethyl)-3-oxo-3,4-dihydro-2H-benzo[b][1,4]oxazin-7-yl)-3-(tert-butyl)urea (20.5 mg, 21.91%) as a white solid. MS (ESI): mass calcd. C22H27N3O4 for: 397.20 m/z, found 398.15[M+H]⁺. 1H NMR (400 MHz, DMSO-d6) δ 8.19 (s, 1H), 7.29-7.42 (m, 2H), 7.19-7.29 (m, 4H), 6.71-6.88 (m, 1H), 6.57-6.71 (m, 1H), 5.94 (s, 1H), 5.10-5.31 (m, 1H), 4.89-5.10 (m, 2H), 3.71-3.81 (m, 2H), 3.30 (s, 3H), 1.25 (s, 9H).

### Example 290: (S)-1-(tert-butyl)-3-(5-(1-(3-chlorophenyl)ethyl)-6-oxo-5,6,7,8-tetrahydro-1,5-naphthyridin-2-yl)urea

### Synthetic Scheme

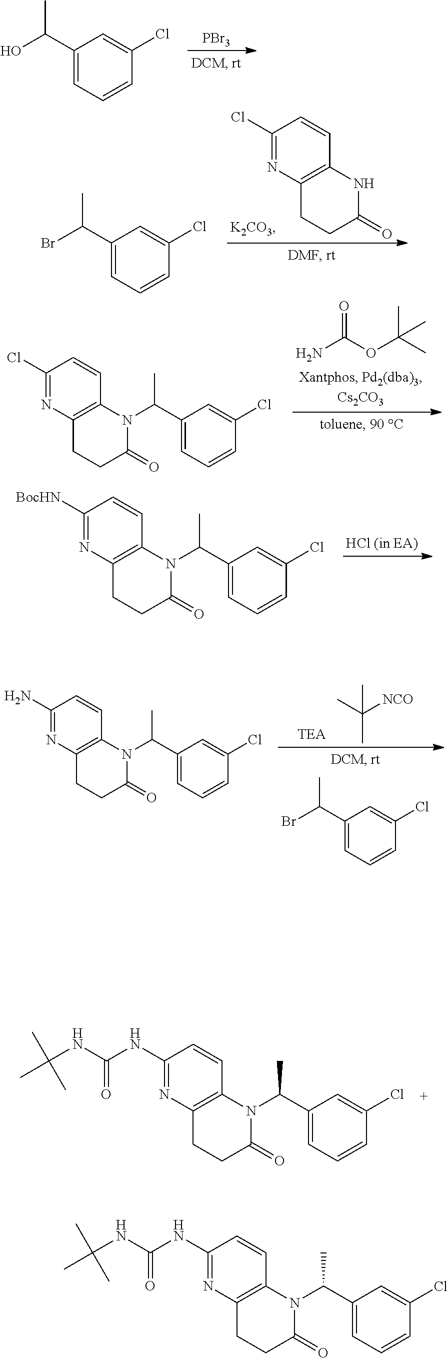

### 1-(1-bromoethyl)-3-chlorobenzene

To a solution of 1-(3-chlorophenyl)ethan-1-ol (500 mg, 3.193 mmol, 1 equiv) in DCM (15 mL) was added PBr₃ (950.6 mg, 3.512 mmol, 1.1 equiv) at 0° C. The resulting mixture was placed at 0° C. and stirred overnight until the starting material was totally consumed by TLC. The reaction mixture was quenched by ice water (10 mL) and extracted with EA (3×30 mL). The organic layers were combined, dried over anhydrous sodium sulfate, filtered and concentrated under reduced pressure. The residue was purified by silica gel column, eluted with PE/EA (3:1) to afford 1-(1-bromoethyl)-3-chlorobenzene (400 mg, 57.08%) as a yellow oil.

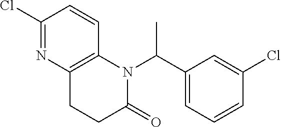 [SMILES: CC(c1cccc(Cl)c1)N1C(=O)CCc2nc(Cl)ccc21]

### 6-chloro-1-(1-(3-chlorophenyl)ethyl)-3,4-dihydro-1,5-naphthyridin-2(1H)-one

To a solution of 1-(1-bromoethyl)-3-chlorobenzene (249.5 mg, 1.367 mmol, 1 equiv) in DMF (10 mL) was added K₂CO₃ (566.6 mg, 4.100 mmol, 3 equiv) and 1-(1-bromoethyl)-3-chlorobenzene (300 mg, 1.367 mmol, 1 equiv). The resulting mixture was placed at rt and stirred overnight until the starting material was totally consumed by LCMS. The reaction mixture was quenched by ice water (10 mL) and extracted with EA (3×30 mL). The organic layers were combined, dried over anhydrous sodium sulfate, filtered and concentrated under reduced pressure. The residue was purified by silica gel column, eluted with PE/EA (3:1) to afford 6-chloro-1-(1-(3-chlorophenyl)ethyl)-3,4-dihydro-1,5-naphthyridin-2(1H)-one (200 mg, 45.26%) as a yellow solid. MS (ESI): mass calcd. for C₁₆H₁₄C₁₂N₂O 320.05 m/z, found 321.05 [M+H]⁺.

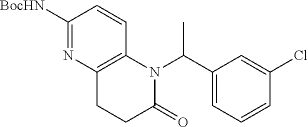 [SMILES: CC(c1cccc(Cl)c1)N1C(=O)CCc2nc(NC(=O)OC(C)(C)C)ccc21]

### tert-butyl (5-(1-(3-chlorophenyl)ethyl)-6-oxo-5,6,7,8-tetrahydro-1,5-naphthyridin-2-yl)carbamate

To a solution of 6-chloro-1-(1-(3-chlorophenyl)ethyl)-3,4-dihydro-1,5-naphthyridin-2(1H)-one (200 mg, 0.623 mmol, 1 equiv) in toluene (15 mL) was added tert-butyl carbamate (87.5 mg, 0.747 mmol, 1.2 equiv), Xantphos (36.0 mg, 0.062 mmol, 0.1 equiv), Pd₂(dba)₃ (57.0 mg, 0.062 mmol, 0.1 equiv) and Cs₂CO₃ (608.6 mg, 1.868 mmol, 3 equiv). The resulting mixture was placed at 90° C. under a nitrogen atmosphere and stirred overnight until the starting material was totally consumed by LCMS. After cooled to room temperature the reaction mixture was quenched by ice water (10 mL) and extracted with EA (3×20 mL). The organic layers were combined, dried over anhydrous sodium sulfate, filtered and concentrated under reduced pressure. The residue was purified by silica gel column, eluted with PE/EA to afford tert-butyl (5-(1-(3-chlorophenyl)ethyl)-6-oxo-5,6,7,8-tetrahydro-1,5-naphthyridin-2-yl)carbamate (140 mg, 53.72%) as a yellow solid. MS (ESI): mass calcd. for C₂₁H₂₄ClN₃O₃, 401.15 m/z, found 402.10 [M+H]⁺.

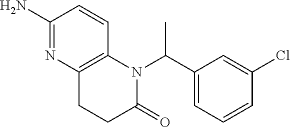 [SMILES: CC(c1cccc(Cl)c1)N1C(=O)CCc2nc(N)ccc21]

### 6-amino-1-(1-(3-chlorophenyl)ethyl)-3,4-dihydro-1,5-naphthyridin-2(1H)-one

To a solution of tert-butyl (5-(1-(3-chlorophenyl)ethyl)-6-oxo-5,6,7,8-tetrahydro-1,5-naphthyridin-2-yl)carbamate (20 mg, 0.050 mmol, 1 equiv) in HCl (in EA) (2 mL). The resulting mixture was placed at and stirred overnight. The reaction mixture was concentrated under vacuum to yield 6-amino-1-(1-(3-chlorophenyl)ethyl)-3,4-dihydro-1,5-naphthyridin-2(1H)-one (80 mg, 60.87%) as a yellow solid. MS (ESI): mass calcd. for C₁₆H₁₆ClN₃₀, 301.10 m/z, found 302.05 [M+H]⁺.

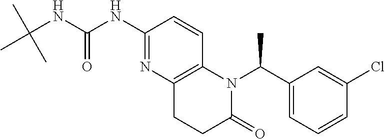 [SMILES: C[C@@H](c1cccc(Cl)c1)N1C(=O)CCc2nc(NC(=O)NC(C)(C)C)ccc21]

### (S)-1-(tert-butyl)-3-(5-(1-(3-chlorophenyl)ethyl)-6-oxo-5,6,7,8-tetrahydro-1,5-naphthyridin-2-yl)urea

To a stirred solution of 6-amino-1-(1-(3-chlorophenyl)ethyl)-3,4-dihydro-1,5-naphthyridin-2(1H)-one (80 mg, 0.265 mmol, 1 equiv) in DCM (10 mL) was added 2-isocyanato-2-methylpropane (131.4 mg, 1.325 mmol, 5 equiv) and TEA (80.4 mg, 0.795 mmol, 3 equiv) stirred overnight at rt. The reaction progress was monitored by LCMS. The reaction mixture was quenched by water (10 mL) and extracted with DCM (3×10 mL). The organic layers were combined, dried over anhydrous sodium sulfate, filtered and concentrated under vacuum. The crude was purified by flash chromatography with PE/EA and Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 45% B to 73% B in 9 min, 73% B; Wave Length: 254/220 nm; RT1(min): 9.6; Number Of Runs: 0) and Chiral Prep-HPLC (Column: CHIRALPAK IF, 2*25 cm, 5 μm; Mobile Phase A: Hex(0.5% 2M NH₃-MeOH)—HPLC, Mobile Phase B: EtOH—HPLC; Flow rate: 20 mL/min; Gradient: 50% B to 50% B in 13 min; Wave Length: 220/254 nm; RT1(min): 9.187; RT2(min): 10.914; Sample Solvent: EtOH—HPLC; Injection Volume: 1.2 mL; Number Of Runs: 4) to afford (S)-1-(tert-butyl)-3-(5-(1-(3-chlorophenyl)ethyl)-6-oxo-5,6,7,8-tetrahydro-1,5-naphthyridin-2-yl)urea (3.6 mg, 3.38%) as a white solid. MS (ESI): mass calcd. for C₂₁H₂₅ClN₄O₂, 400.17 m/z, found 435.10 [M+H]⁺. ¹H NMR (300 MHz, DMSO-d₆) δ 8.88 (s, 1H), 8.00 (s, 1H), 7.28-7.42 (m, 3H), 7.23 (d, J=7.5 Hz, 1H), 6.99 (q, J=9.0 Hz, 2H), 6.07 (q, J=7.1 Hz, 1H), 2.90-3.02 (m, 2H), 2.76-2.82 (m, 1H), 2.63-2.70 (m, 1H), 1.68 (d, J=7.2 Hz, 3H), 1.30 (s, 9H). And another isomer to afford (R)-1-(tert-butyl)-3-(5-(1-(3-chlorophenyl)ethyl)-6-oxo-5,6,7,8-tetrahydro-1,5-naphthyridin-2-yl)urea (5.2 mg, 4.89%) as a white solid. MS (ESI): mass calcd. for C₂₁H₂₅ClN₄O₂, 400.17 m/z, found 435.10 [M+H]⁺. ¹H NMR (300 MHz, DMSO-d₆) δ 8.88 (s, 1H), 8.00 (s, 1H), 7.28-7.42 (m, 3H), 7.23 (d, J=7.5 Hz, 1H), 6.99 (q, J=9.0 Hz, 2H), 6.07 (q, J=7.1 Hz, 1H), 2.90-3.02 (m, 2H), 2.76-2.82 (m, 1H), 2.63-2.70 (m, 1H), 1.68 (d, J=7.2 Hz, 3H), 1.30 (s, 9H).

### Example 291: (R)-1-(tert-butyl)-3-(5-(1-(3-chlorophenyl)ethyl)-6-oxo-5,6,7,8-tetrahydro-1,5-naphthyridin-2-yl)urea

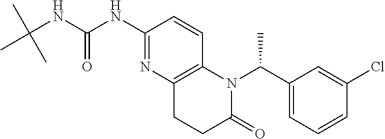 [SMILES: C[C@H](c1cccc(Cl)c1)N1C(=O)CCc2nc(NC(=O)NC(C)(C)C)ccc21]

### (R)-1-(tert-butyl)-3-(5-(1-(3-chlorophenyl)ethyl)-6-oxo-5,6,7,8-tetrahydro-1,5-naphthyridin-2-yl)urea

To a stirred solution of 6-amino-1-(1-(3-chlorophenyl)ethyl)-3,4-dihydro-1,5-naphthyridin-2(1H)-one (80 mg, 0.265 mmol, 1 equiv) in DCM (10 mL) was added 2-isocyanato-2-methylpropane (131.4 mg, 1.325 mmol, 5 equiv) and TEA (80.4 mg, 0.795 mmol, 3 equiv) stirred overnight at rt. The reaction progress was monitored by LCMS. The reaction mixture was quenched by water (10 mL) and extracted with DCM (3×10 mL). The organic layers were combined, dried over anhydrous sodium sulfate, filtered and concentrated under vacuum. The crude was purified by flash chromatography with PE/EA and Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 45% B to 73% B in 9 min, 73% B; Wave Length: 254/220 nm; RT1(min): 9.6; Number Of Runs: 0) and Chiral Prep-HPLC (Column: CHIRALPAK IF, 2*25 cm, 5 μm; Mobile Phase A: Hex(0.5% 2M NH₃-MeOH)—HPLC, Mobile Phase B: EtOH—HPLC; Flow rate: 20 mL/min; Gradient: 50% B to 50% B in 13 min; Wave Length: 220/254 nm; RT1(min): 9.187; RT2(min): 10.914; Sample Solvent: EtOH—HPLC; Injection Volume: 1.2 mL; Number Of Runs: 4) to afford (S)-1-(tert-butyl)-3-(5-(1-(3-chlorophenyl)ethyl)-6-oxo-5,6,7,8-tetrahydro-1,5-naphthyridin-2-yl)urea (3.6 mg, 3.38%) as a white solid. MS (ESI): mass calcd. for C₂₁H₂₅ClN₄O₂, 400.17 m/z, found 435.10 [M+H]⁺. ¹H NMR (300 MHz, DMSO-d₆) δ 8.88 (s, 1H), 8.00 (s, 1H), 7.28-7.42 (m, 3H), 7.23 (d, J=7.5 Hz, 1H), 6.99 (q, J=9.0 Hz, 2H), 6.07 (q, J=7.1 Hz, 1H), 2.90-3.02 (m, 2H), 2.76-2.82 (m, 1H), 2.63-2.70 (m, 1H), 1.68 (d, J=7.2 Hz, 3H), 1.30 (s, 9H). And another isomer to afford (R)-1-(tert-butyl)-3-(5-(1-(3-chlorophenyl)ethyl)-6-oxo-5,6,7,8-tetrahydro-1,5-naphthyridin-2-yl)urea (5.2 mg, 4.89%) as a white solid. MS (ESI): mass calcd. for C₂₁H₂₅ClN₄O₂, 400.17 m/z, found 435.10 [M+H]⁺. ¹H NMR (300 MHz, DMSO-d₆) δ 8.88 (s, 1H), 8.00 (s, 1H), 7.28-7.42 (m, 3H), 7.23 (d, J=7.5 Hz, 1H), 6.99 (q, J=9.0 Hz, 2H), 6.07 (q, J=7.1 Hz, 1H), 2.90-3.02 (m, 2H), 2.76-2.82 (m, 1H), 2.63-2.70 (m, 1H), 1.68 (d, J=7.2 Hz, 3H), 1.30 (s, 9H).

### Example 292: 1-(4-benzyl-2,2-dimethyl-3-oxo-3,4-dihydro-2H-benzo[b][1,4]oxazin-7-yl)-3-(tert-butyl)urea

### Synthetic Scheme

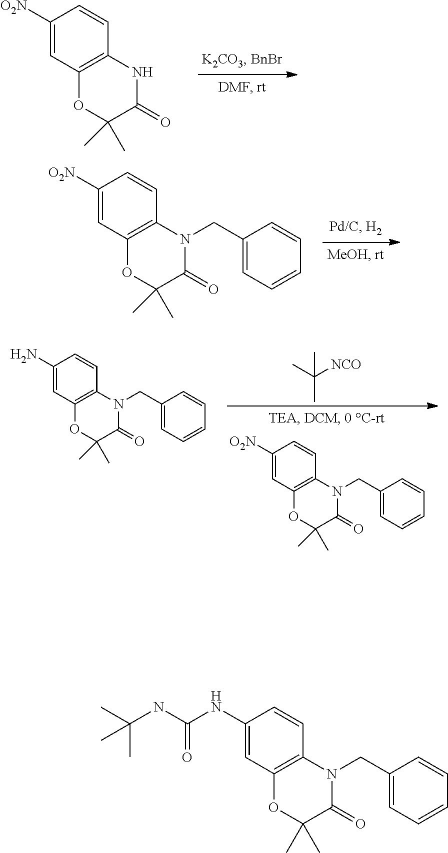

### 4-benzyl-2,2-dimethyl-7-nitro-2H-benzo[b][1,4]oxazin-3(4H)-one

To a stirred solution of 2,2-dimethyl-7-nitro-2H-benzo[b][1,4]oxazin-3(4H)-one (300 mg, 1.350 mmol, 1 equiv) in anhydrous DMF (10 mL) was added K₂CO (373 mg, 2.700 mmol, 2 equiv) and BnBr (346 mg, 2.025 mmol, 1.5 equiv) at rt. The reaction mixture was stirred for 1.5 h at rt. After completion of reaction, the reaction mixture was quenched by addition of water (15 mL). The aqueous layer was extracted with ethyl acetate (3×30 mL). The combined organic phase was washed with brine (20 mL), dried over anhydrous sodium sulfate filtered and concentrated under reduced pressure. The residue was purified by silica gel column, eluted with PE/EA (3:1) to give 4-benzyl-2,2-dimethyl-7-nitro-2H-benzo[b][1,4]oxazin-3(4H)-one (420 mg, 99.60%) as a yellow oil. MS (ESI): mass calcd. for C₁₇H₁₆N₂O₄: 312.11, found: 313.20 [M+H]⁺.

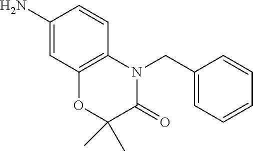 [SMILES: CC1(C)Oc2cc(N)ccc2N(Cc2ccccc2)C1=O]

### 7-amino-4-benzyl-2,2-dimethyl-2H-benzo[b][1,4]oxazin-3(4H)-one

To a solution of 4-benzyl-2,2-dimethyl-7-nitro-2H-benzo[b][1,4]oxazin-3(4H)-one (420 mg, 1.345 mmol, 1 equiv) in MeOH (15 mL) was added Pd/C under N₂ atmosphere. The reaction was stirred at room temperature for 2.5 h under H₂ atmosphere. The reaction was filtered. The filtrate was concentrated to give 7-amino-4-benzyl-2,2-dimethyl-2H-benzo[b][1,4]oxazin-3(4H)-one (340 mg, 87.11%) as a white solid. MS (ESI): mass calcd. for C₁₇H₁₈N₂O₂: 282.14, found: 283.10[M+H]⁺.

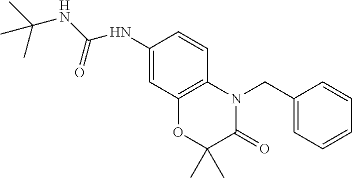 [SMILES: CC(C)(C)NC(=O)Nc1ccc2c(c1)OC(C)(C)C(=O)N2Cc1ccccc1]

### 1-(4-benzyl-2,2-dimethyl-3-oxo-3,4-dihydro-2H-benzo[b][1,4]oxazin-7-yl)-3-(tert-butyl)urea

To a mixture of 7-amino-4-benzyl-2,2-dimethyl-2H-benzo[b][1,4]oxazin-3(4H)-one (110 mg, 0.390 mmol, 1 equiv) and TEA (236 mg, 2.340 mmol, 6 equiv) in DCM (5 mL) was added 2-isocyanato-2-methylpropane (232 mg, 2.340 mmol, 6 equiv) at 0° C. The reaction was stirred for overnight at rt. The reaction mixture was quenched by water (10 mL) and extracted with DCM (3×10 mL). The organic layers were combined, dried over anhydrous sodium sulfate, filtered and concentrated under vacuum. The residue was purified by silica gel column (PE/EA 1:1) and Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 17% B to 42% B in 9 min, 42% B; Wave Length: 254/220 nm) to give 1-(4-benzyl-2,2-dimethyl-3-oxo-3,4-dihydro-2H-benzo[b][1,4]oxazin-7-yl)-3-(tert-butyl)urea (21.7 mg, 14.53%) as a white solid. MS (ESI): mass calcd. for C₂₂H₂₇N₃O₃, 381.21 m/z, found 382.10 [M+H]⁺. ¹H NMR (400 MHz, DMSO-d₆) δ 8.20 (s, 1H), 7.31-7.35 (m, 2H), 7.20-7.26 (m, 4H), 6.83 (d, J=8.7 Hz, 1H), 6.72 (dd, J=8.7, 2.3 Hz, 1H), 5.94 (s, 1H), 5.08 (s, 2H), 1.46 (s, 6H), 1.26 (s, 9H).

### Example 293: 1-(tert-butyl)-3-(4-(1-(5-chloro-2-cyanophenyl)ethyl)-3-oxo-3,4-dihydro-2H-benzo[b][1,4]oxazin-7-yl)urea

### Synthetic Scheme

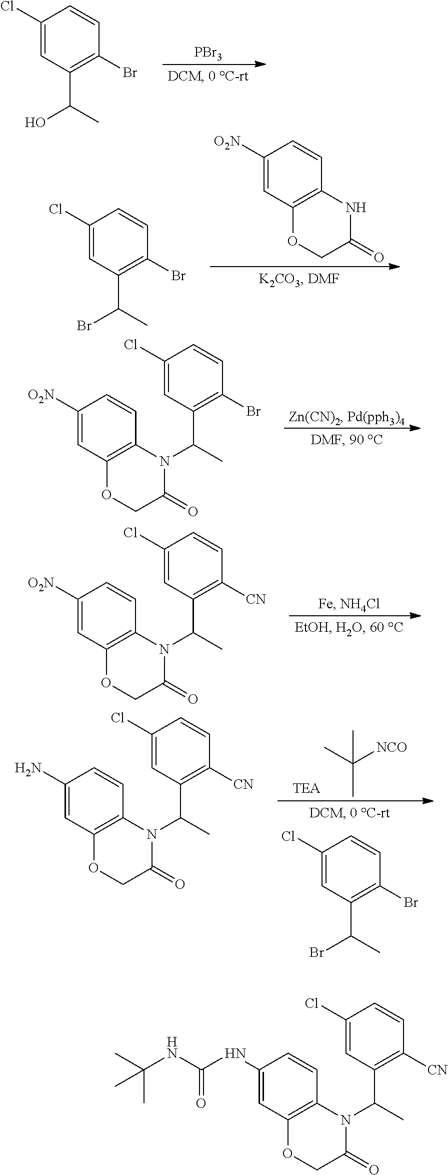

### 1-bromo-2-(1-bromoethyl)-4-chlorobenzene

To a mixture of 1-(2-bromo-5-chlorophenyl)ethan-1-ol (300 mg, 1.047 mmol, 1 equiv) in DCM (5 mL) was added PBr₃ (567 mg, 2.094 mmol, 2 equiv) at 0° C. The reaction was stirred for overnight at rt. The reaction mixture was quenched by ice water (10 mL) and the pH value of the aqueous phase was adjusted to 8 with NaHCO₃(aq.). The mixture was extracted with DCM (3×10 mL). The organic layers were combined, dried over anhydrous sodium sulfate, filtered and concentrated under vacuum. The residue was purified by silica gel column, eluted with PE/EA (20:1) to give 1-bromo-2-(1-bromoethyl)-4-chlorobenzene (218 mg, 69.76%) as yellow oil.

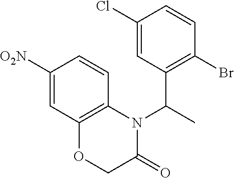 [SMILES: CC(c1cc(Cl)ccc1Br)N1C(=O)COc2cc([N+](=O)[O-])ccc21]

### 4-(1-(2-bromo-5-chlorophenyl)ethyl)-7-nitro-2H-benzo[b][1,4]oxazin-3(4H)-one

To a mixture of 7-nitro-2H-benzo[b][1,4]oxazin-3(4H)-one (87 mg, 0.447 mmol, 1 equiv) and K₂CO₃ (187 mg, 1.340 mmol, 3 equiv) in DMF (5 mL). 1-Bromo-2-(1-bromoethyl)-4-chlorobenzene (200 mg, 0.670 mmol, 1.5 equiv) was added into the solution. The resulting solution was stirred for overnight at rt. The reaction mixture was quenched by water (10 mL) and extracted with DCM (3×10 mL). The organic layers were combined, dried over anhydrous sodium sulfate, filtered and concentrated under vacuum The residue was purified by silica gel column, eluted with PE/EA (3:1) to afford 4-(1-(2-bromo-5-chlorophenyl)ethyl)-7-nitro-2H-benzo[b][1,4]oxazin-3(4H)-one (150 mg, 81.55%) as a yellow solid.

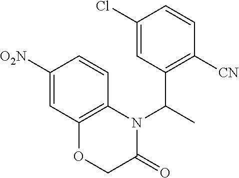 [SMILES: CC(c1cc(Cl)ccc1C#N)N1C(=O)COc2cc([N+](=O)[O-])ccc21]

### 4-chloro-2-(1-(7-nitro-3-oxo-2,3-dihydro-4H-benzo[b][1,4]oxazin-4-yl)ethyl)benzonitrile

A solution of 4-(1-(2-bromo-5-chlorophenyl)ethyl)-7-nitro-2H-benzo[b][1,4]oxazin-3(4H)-one (150 mg, 0.360 mmol, 1 equiv) in DMF (3 mL) was added Zn(CN)₂ (106 mg, 0.90 mmol, 2.5 equiv) and Pd(PPh₃)₄ (42 mg, 0.036 mmol, 0.1 equiv) at rt. The resulting mixture was maintained under nitrogen and stirred at 90° C. for overnight. After cooling down to rt, the reaction was quenched with water (10 mL). The resulting mixture was extracted with ethyl acetate (3×10 mL). The organic layers were combined, dried over anhydrous sodium sulfate, filtered and concentrated. The residue obtained was purified by silica gel chromatography eluted with PE/EA (2:1) to afford 4-chloro-2-(1-(7-nitro-3-oxo-2,3-dihydro-4H-benzo[b][1,4]oxazin-4-yl)ethyl)benzonitrile (100 mg, 77.52%). LC/MS: mass calcd. for C₁₇H₁₂ClN₃O₄: 357.05, found: 358.00 [M+H]⁺.

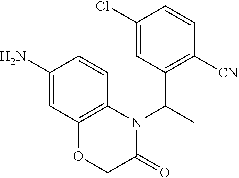 [SMILES: CC(c1cc(Cl)ccc1C#N)N1C(=O)COc2cc(N)ccc21]

### 2-(1-(7-amino-3-oxo-2,3-dihydro-4H-benzo[b][1,4]oxazin-4-yl)ethyl)-4-chlorobenzonitrile

To a mixture of 4-chloro-2-(1-(7-nitro-3-oxo-2,3-dihydro-4H-benzo[b][1,4]oxazin-4-yl)ethyl)benzonitrile (50 mg, 0.140 mmol, 1 equiv) in EtOH (10 mL) and water (2 mL). Fe (78 mg, 1.400 mmol, 10 equiv) and NH₄C₁ (74.76 mg, 1.400 mmol, 10 equiv) were added into the mixture. The resulting solution was stirred at 60° C. for 1 h. The resulting mixture was concentrated under vacuum. The residue was purified by flash chromatography with PE/EA (1:1) to afford 2-(1-(7-amino-3-oxo-2,3-dihydro-4H-benzo[b][1,4]oxazin-4-yl)ethyl)-4-chlorobenzonitrile (33 mg, 72.04%) as a yellow solid. LC/MS: mass calcd. for C₁₇H₁₄ClN₃O₂, 327.08 m/z, found 328.15 [M+H]⁺.

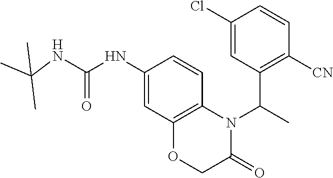 [SMILES: CC(c1cc(Cl)ccc1C#N)N1C(=O)COc2cc(NC(=O)NC(C)(C)C)ccc21]

### 1-(tert-butyl)-3-(4-(1-(5-chloro-2-cyanophenyl)ethyl)-3-oxo-3,4-dihydro-2H-benzo[b][1,4]oxazin-7-yl)urea

To a mixture of 2-(1-(7-amino-3-oxo-2,3-dihydro-4H-benzo[b][1,4]oxazin-4-yl)ethyl)-4-chlorobenzonitrile (33 mg, 0.101 mmol, 1 equiv) and TEA (21 mg, 0.202 mmol, 2 equiv) in DCM (4 mL) was added 2-isocyanato-2-methylpropane (30 mg, 0.303 mmol, 3 equiv) at 0° C. The reaction was stirred for overnight at rt. The reaction mixture was quenched by water (10 mL) and extracted with DCM (3×10 mL). The organic layers were combined, dried over anhydrous sodium sulfate, filtered and concentrated. The crude product was purified by silica gel column with PE/EA (1:1) and Prep-HPLC (Column: XBridge Prep Phenyl OBD Column, 19*150 mm, 5 μm; Mobile Phase A: Water (50 mmol/L NH₄HCO₃), Mobile Phase B: ACN; Flow rate: 60 mL/min; Gradient: 35% B to 57% B in 10 min, 57% B; Wave Length: 254/220 nm) to give 1-(tert-butyl)-3-(4-(1-(5-chloro-2-cyanophenyl)ethyl)-3-oxo-3,4-dihydro-2H-benzo[b][1,4]oxazin-7-yl)urea (4.7 mg, 10.92%) as a white solid. LC/MS: mass calcd. for C₂₂H₂₃ClN₄O₃, 426.15 m/z, found 427.05 [M+H]⁺. ¹H NMR (300 MHz, DMSO-d₆) δ 8.21 (s, 1H), 7.79-7.84 (m, 2H), 7.58 (dd, J=8.3, 2.0 Hz, 1H), 7.13 (d, J=2.3 Hz, 1H), 6.88 (d, J=8.9 Hz, 1H), 6.77 (dd, J=8.8, 2.4 Hz, 1H), 5.91-5.93 (m, 2H), 4.54 (s, 2H), 1.82 (d, J=7.0 Hz, 3H), 1.26 (s, 9H).

### Example 294: 1-(3-amino-1-benzyl-2-oxo-1,2-dihydroquinoxalin-6-yl)-3-(tert-butyl)urea

### Synthetic Scheme

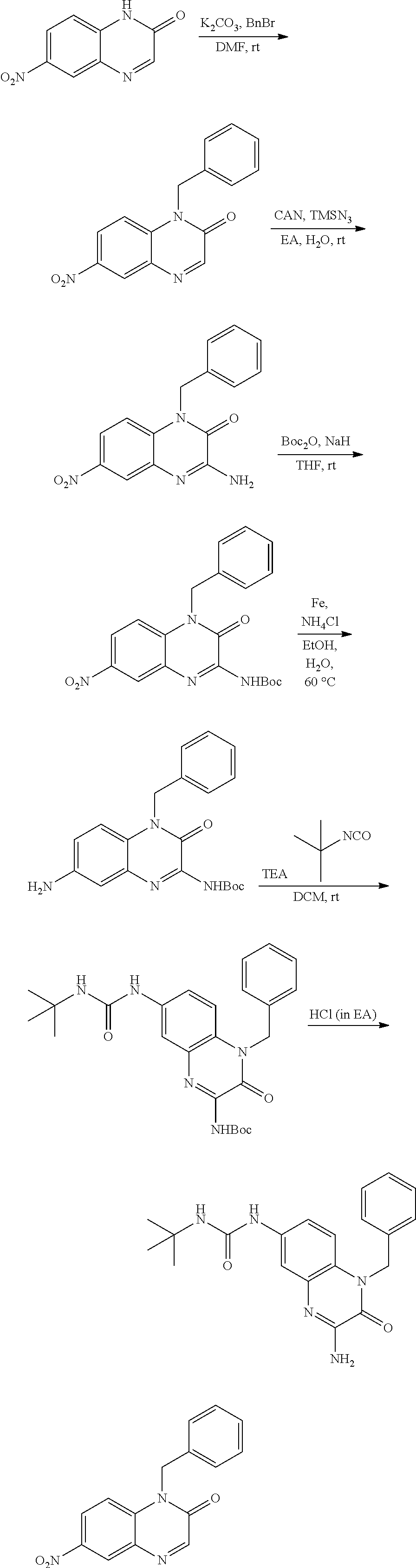

### 1-benzyl-6-nitroquinoxalin-2(1H)-one

To a solution of 6-nitro-1H-quinoxalin-2-one (3 g, 15.695 mmol, 1 equiv) in DMF (50 mL) was added K₂CO₃ (6.51 g, 47.104 mmol, 3 equiv) and BnBr (4.03 g, 23.562 mmol, 1.5 equiv). The resulting mixture was placed at rt and stirred overnight until the starting material was totally consumed by LCMS, The reaction mixture was quenched by water (10 mL) and extracted with EA (3×50 mL). The combined organic layers were washed with NaCl (30 mL), dried over anhydrous Na₂SO₄. After filtration, the filtrate was concentrated under reduced pressure. The residue was purified by silica gel column chromatography, eluted with PE/EA (3/1) to afford 1-benzyl-6-nitroquinoxalin-2(1H)-one (3 g, 65.14%) as a yellow solid. MS (ESI): mass calcd. for C₁₅H₁₁N₃O₃ 281.08 m/z, found 282.05 [M+H]⁺.

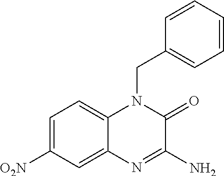 [SMILES: Nc1nc2cc([N+](=O)[O-])ccc2n(Cc2ccccc2)c1=O]

### 3-amino-1-benzyl-6-nitroquinoxalin-2(1H)-one

An oven-dried Schlenk tube was charged with 1-benzyl-6-nitroquinoxalin-2(1H)-one (1 g, 3.555 mmol, 1.0 equiv), CAN (2.93 g, 5.325 mmol, 1.5 equiv) and a magnetic stirring bar, and then was purged with nitrogen for three times. EtOAc (36 mL) and H₂O (128.1 mg, 7.111 mmol, 2.0 equiv) were added in turn via respective syringes, and the mixture was stirred at 20° C. in water bath. Then, TMSN₃ (1.23 g, 10.676 mmol, 3.0 equiv) was added in turn via a syringe, and the mixture was stirred under the previous conditions until the substrate was consumed (monitored by TLC, about 10 hour). The mixture was added with CH₂Cl₂ (200 mL) and saturated aqueous Na₂CO₃ (200 mL). The organic layer was isolated and the remaining aqueous phase was further extracted with CH₂Cl₂ (150 mL×2). The combined organic phase was washed with 0.3 M HCl (100 mL×2), and adjusted pH to 8 by the addition of saturated aqueous Na₂CO₃, followed by extraction with CH₂Cl₂ (150 mL×3). The received organic phase was washed with saturated brine (200 mL). The organic layer was dried over Na₂SO₄, and then filtered. The filtrate was evaporated under reduced pressure, and the resulting residue was purified by column chromatography on neutral aluminum oxide (petroleum ether/ethyl acetate, in some cases, 5-10 vol % triethylamine is helpful.) to afford 3-amino-1-benzyl-6-nitroquinoxalin-2(1H)-one (300 mg, 24.48%) as a yellow solid. MS (ESI): mass calcd. for C₁₅H₁₂N₄O₃, 296.09 m/z, found 297.10 [M+H]⁺.

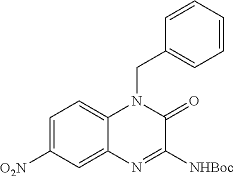 [SMILES: CC(C)(C)OC(=O)Nc1nc2cc([N+](=O)[O-])ccc2n(Cc2ccccc2)c1=O]

### tert-butyl (4-benzyl-7-nitro-3-oxo-3,4-dihydroquinoxalin-2-yl)carbamate

In a 100 mL round bottom flask, to a solution of 3-amino-1-benzyl-6-nitroquinoxalin-2(1H)-one (300 mg, 1.013 mmol, 1 equiv) in THF (10 mL) was added 60% NaH (60.7 mg, 1.519 mmol, 1.5 equiv) at 0° C. under a nitrogen atmosphere. The reaction mixture was stirred at 0° C. for 0.5 h. Then a solution of Boc₂O (331.4 mg, 1.519 mmol, 1.5 equiv) in 5 mL THF was added dropwise and the mixture was stirred for another 120 mins. The reaction was quenched with water/sat. NH₄Cl (40 mL), and then the mixture was extracted with ether/EtOAc (2×30 mL). The combined organic extracts were washed with brine (20 mL), dried over anhydrous Na₂SO₄, and concentrated under vacuum to yield a crude product which was directly purified by flash chromatography (0-100% ethyl acetate/petroleum ether) to afford tert-butyl (4-benzyl-7-nitro-3-oxo-3,4-dihydroquinoxalin-2-yl)carbamate (200 mg, 49.45%) as a yellow solid. MS (ESI): mass calcd. for C₂₀H₂₀N₄O₅, 396.14 m/z, found 397.10 [M+H]⁺.

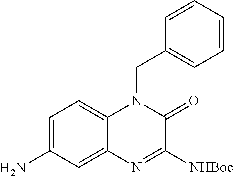 [SMILES: CC(C)(C)OC(=O)Nc1nc2cc(N)ccc2n(Cc2ccccc2)c1=O]

### tert-butyl (7-amino-4-benzyl-3-oxo-3,4-dihydroquinoxalin-2-yl)carbamate

To a solution of tert-butyl (4-benzyl-7-nitro-3-oxo-3,4-dihydroquinoxalin-2-yl)carbamate (200 mg, 0.505 mmol, 1 equiv) in EtOH (10 mL) and H₂O (2.5 mL) was added Fe (281.7 mg, 5.045 mmol, 10 equiv) and NH₄C₁ (269.8 mg, 5.045 mmol, 10 equiv). The resulting mixture was stirred for overnight at 60° C. The reaction progress was monitored by LCMS, the reaction mixture was quenched by water and extracted with EA (3×20 mL). The combined organic extracts were washed with brine (20 mL), dried over anhydrous Na₂SO₄, and concentrated under vacuum to yield a crude product which was directly purified by flash chromatography (0-100% ethyl acetate/petroleum ether) to afford tert-butyl (7-amino-4-benzyl-3-oxo-3,4-dihydroquinoxalin-2-yl)carbamate (100 mg, 46.83%) as a yellow solid. MS (ESI): mass calcd. for C₂₀H₂₂N₄O₃, 366.17 m/z, found 367.10 [M+H]⁺.

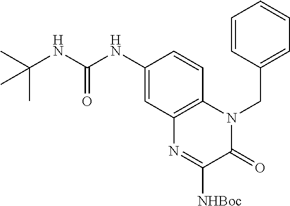 [SMILES: CC(C)(C)NC(=O)Nc1ccc2c(c1)nc(NC(=O)OC(C)(C)C)c(=O)n2Cc1ccccc1]

### tert-butyl (4-benzyl-7-(3-(tert-butyl)ureido)-3-oxo-3,4-dihydroquinoxalin-2-yl)carbamate

To a stirred solution of tert-butyl (7-amino-4-benzyl-3-oxo-3,4-dihydroquinoxalin-2-yl)carbamate (90 mg, 0.246 mmol, 1 equiv) in DCM (5 mL) was added 2-isocyanato-2-methylpropane (121.7 mg, 1.228 mmol, 5 equiv) and TEA (74.5 mg, 0.737 mmol, 3 equiv) stirred overnight at rt. The reaction progress was monitored by LCMS. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by flash chromatography (0-100% ethyl acetate/petroleum ether) to afford tert-butyl (4-benzyl-7-(3-(tert-butyl)ureido)-3-oxo-3,4-dihydroquinoxalin-2-yl)carbamate (30 mg, 25.68%) as a yellow solid. MS (ESI): mass calcd. for C₂₅H₃₁N₅O₄, 465.24 m/z, found 466.15 [M+H]⁺.

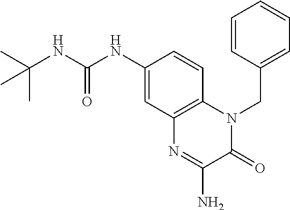 [SMILES: CC(C)(C)NC(=O)Nc1ccc2c(c1)nc(N)c(=O)n2Cc1ccccc1]

### 1-(3-amino-1-benzyl-2-oxo-1,2-dihydroquinoxalin-6-yl)-3-(tert-butyl)urea

To a stirred solution of tert-butyl (4-benzyl-7-(3-(tert-butyl)ureido)-3-oxo-3,4-dihydroquinoxalin-2-yl)carbamate (30 mg, 0.064 mmol, 1 equiv) in HCl (in EA) (10 mL) stirred 2 h at rt. The reaction mixture was concentrated under vacuum to yield a crude product which was directly purified by flash chromatography (0-100% ethyl acetate/petroleum ether) to afford 30 mg crude product as a white solid. The compound was separated by preparative HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 45% B to 73% B in 9 min, 73% B; Wave Length: 254/220 nm; RT1(min): 9.6; Number Of Runs: 0) to afford 1-(3-amino-1-benzyl-2-oxo-1,2-dihydroquinoxalin-6-yl)-3-(tert-butyl)urea (9.3 mg, 29.89%) as a yellow solid. MS (ESI): mass calcd. for C₂₀H₂₃N₅O2, 365.19 m/z, found 366.10 [M+H]⁺. ¹H NMR (300 MHz, DMSO-d₆) δ 8.55 (br, 2H), 8.43 (s, 1H), 7.69 (d, J=2.3 Hz, 1H), 7.21-7.37 (m, 5H), 7.15 (d, J=9.1 Hz, 1H), 7.04 (dd, J=9.0, 2.4 Hz, 1H), 6.03 (s, 1H), 5.44 (s, 2H), 1.28 (s, 9H).

### Example 295: 1-(tert-butyl)-3-(3-ethyl-2-oxo-1-(3-(trifluoromethoxy)benzyl)-1,2-dihydroquinoxalin-6-yl)urea

### Synthetic Scheme

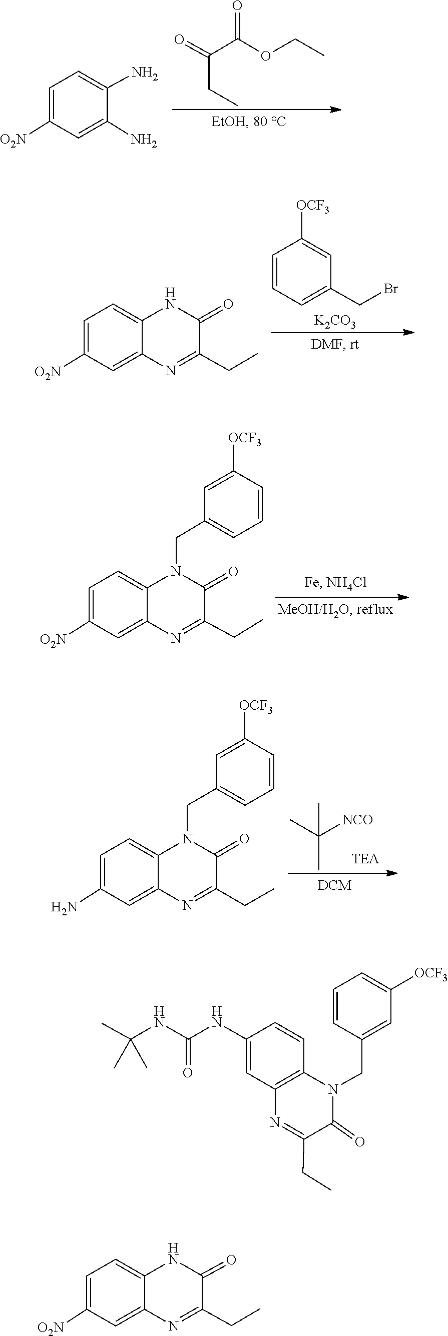

### 3-ethyl-6-nitroquinoxalin-2(1H)-one

To a solution of 2-amino-4-nitroaniline (2 g, 13.060 mmol, 1 equiv) in ethyl alcohol (30 mL) was added ethyl 2-oxobutanoate (2.039 g, 15.667 mmol, 1.20 equiv). The reaction was stirred at 80° C. for 3 h. The mixture was allowed to cool down to rt. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give tert-butyl 3-ethyl-6-nitro-1H-quinoxalin-2-one (2.5 g, 87.33%) as a red solid. MS (ESI): mass calcd. for C₁₀H₉N₃O₃: 219.06 m/z, found 220.10 [M+H]⁺

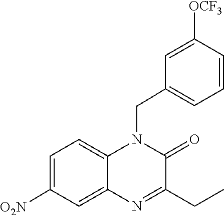 [SMILES: CCc1nc2cc([N+](=O)[O-])ccc2n(Cc2cccc(OC(F)(F)F)c2)c1=O]

### 3-ethyl-6-nitro-1-(3-(trifluoromethoxy)benzyl)quinoxalin-2(1H)-one

To a solution of 3-ethyl-6-nitro-1H-quinoxalin-2-one (700 mg, 3.193 mmol, 1 equiv) and 1-(bromomethyl)-3-(trifluoromethoxy)benzene (2443.30 mg, 9.579 mmol, 3 equiv) in DMF (10 mL) was added K₂CO₃ (1333.70 mg, 9.579 mmol, 3 equiv). The reaction was stirred at rt for 3 h. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 3-ethyl-6-nitro-1-{[3-(trifluoromethoxy)phenyl]methyl}quinoxalin-2-one (819 mg, 65.20%) as a red solid. LC/MS: mass calcd. for C₁₈H₁₄F₃N₃O₄: 393.09, found: 394.05 [M+H]⁺.

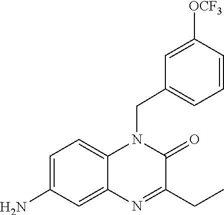 [SMILES: CCc1nc2cc(N)ccc2n(Cc2cccc(OC(F)(F)F)c2)c1=O]

### 6-amino-3-ethyl-1-(3-(trifluoromethoxy)benzyl)quinoxalin-2(1H)-one

To a solution of 3-ethyl-6-nitro-1-{[3-(trifluoromethoxy)phenyl]methyl}quinoxalin-2-one (400 mg, 1.017 mmol, 1 equiv) in methanol (15 mL) and water (1.5 mL) was added iron (567.93 mg, 10.170 mmol, 10 equiv) and NH₄C₁ (543.98 mg, 10.170 mmol, 10 equiv). The reaction was stirred at 60° C. for 3 h. The mixture was allowed to cool down to rt. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 6-amino-3-ethyl-1-{[3-(trifluoromethoxy)phenyl]methyl}quinoxalin-2-one (300 mg, 81.19%) as a white solid. LC/MS (ESI): mass calcd. for C₁₈H₁₆F₃N₃O₂: 363.12 m/z, found: 364.05 [M+H]⁺.

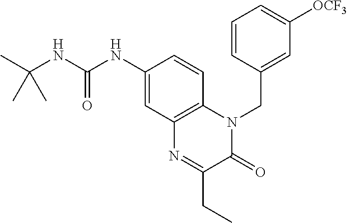 [SMILES: CCc1nc2cc(NC(=O)NC(C)(C)C)ccc2n(Cc2cccc(OC(F)(F)F)c2)c1=O]

### 1-(tert-butyl)-3-(3-ethyl-2-oxo-1-(3-(trifluoromethoxy)benzyl)-1,2-dihydroquinoxalin-6-yl)urea

To a solution of 6-amino-3-ethyl-1-{[3-(trifluoromethoxy)phenyl]methyl}quinoxalin-2-one (100 mg, 0.275 mmol, 1 equiv) and 2-isocyanato-2-methylpropane (81.85 mg, 0.825 mmol, 3 equiv) in DCM (3 mL) was added TEA (83.55 mg, 0.825 mmol, 3 equiv). The reaction was stirred at rt for 3 h. Quenched with water (10 mL) and extracted with DCM (3×10 mL). The combined extracts were washed with water, brine, dried over Na₂SO₄, filtered and concentrated. The residue was purified by preparative HPLC using a XBridge Prep OBD C18 Column, 30*150 mm, 5 μm (eluent: 17% to 42% (v/v) CH₃CN and H₂O with 10 mmol/L NH₄HCO₃) to afford the title compound 3-tert-butyl-1-(3-ethyl-2-oxo-1-{[3-(trifluoromethoxy)phenyl]methyl}quinoxalin-6-yl)urea (25.1 mg, 19.69%) as a white solid. LC/MS (ESI): mass calcd. for C₂₃H₂₅F₃N₄O₃:462.19 m/z, found: 461.00 [M−H]⁻. ¹⁹F NMR (282 MHz, DMSO) δ −56.69. ¹H NMR (300 MHz, DMSO-d₆) δ 8.40 (s, 1H), 7.96 (m, 1H), 7.45 (m, 1H), 7.18-7.38 (m, 5H), 6.03 (s, 1H), 5.50 (s, 2H), 2.89 (m, 2H), 1.26-1.30 (m, 12H).

### Example 296: 1-(tert-butyl)-3-(3-oxo-4-(2,2,2-trifluoro-1-phenylethyl)-3,4-dihydro-2H-benzo[b][1,4]oxazin-7-yl)urea

### Synthetic Scheme

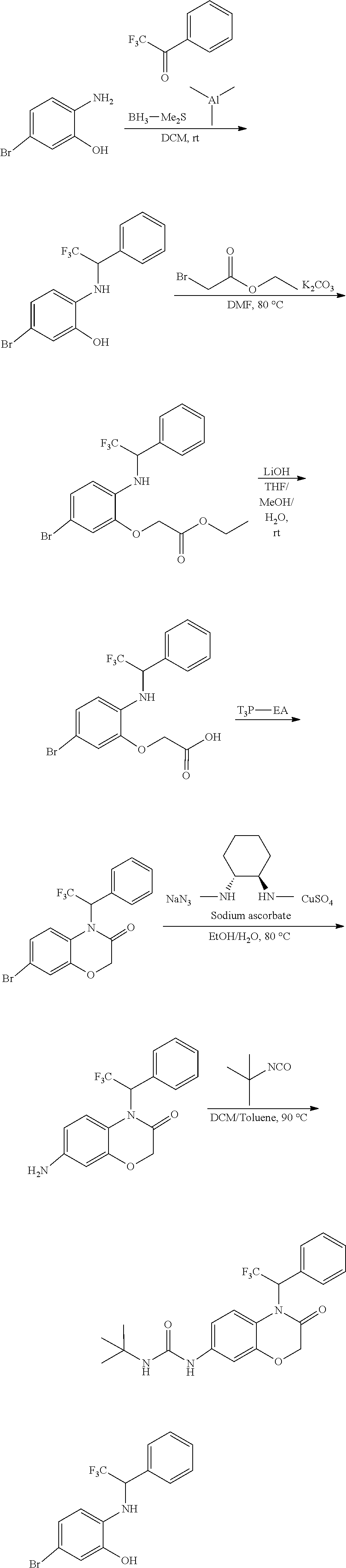

### 5-bromo-2-((2,2,2-trifluoro-1-phenylethyl)amino)phenol

To a solution of 2-amino-5-bromophenol (1 g, 5.318 mmol, 1 equiv) and trifluoroacetophenone (0.93 g, 5.318 mmol, 1 equiv) in DCM (20 mL) was added trimethylaluminium (1.15 g, 15.954 mmol, 3 equiv) under N₂. The reaction was stirred at rt under N₂ for 15 h. To the above mixture was added BH₃-Me₂S (10.64 mL, 10.636 mmol, 2 equiv, 1.0 M in THF). The resulting mixture was stirred for additional 2 h at rt. The reaction was quenched by dropwise addition of 20% aqueous NaOH and the aqueous layer was extracted with DCM (3×100 mL). The combined extracts were washed with water, brine, dried over Na₂SO₄, filtered and concentrated. The residue was purified by column chromatography on silica gel with EA/PE (0-100%) to give 5-bromo-2-[(2,2,2-trifluoro-1-phenylethyl)amino]phenol (1.5 g, 81.48%) as an orange oil. MS (ESI): mass calcd. for C₁₄H₁₁BrF₃NO: 345.00 m/z, found 346.05, 348.05 [M+H, M+H+2]*.

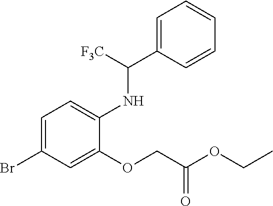 [SMILES: CCOC(=O)COc1cc(Br)ccc1NC(c1ccccc1)C(F)(F)F]

### ethyl 2-(5-bromo-2-((2,2,2-trifluoro-1-phenylethyl)amino)phenoxy)acetate

To a solution of 5-bromo-2-[(2,2,2-trifluoro-1-phenylethyl)amino]phenol (400 mg, 1.156 mmol, 1 equiv) in DMF/H₂O (10:1, 11 mL) was added ethyl bromoacetate (231.58 mg, 1.387 mmol, 1.2 equiv) and K₂CO₃ (321.74 mg, 2.312 mmol, 2 equiv). The reaction was stirred at 80° C. for 1 h. After cooling down to rt, the reaction was quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give ethyl 2-{5-bromo-2-[(2,2,2-trifluoro-1-phenylethyl)amino]phenoxy}acetate (400 mg, 80.08%) as an orange solid. MS (ESI): mass calcd. for C₁₈H₁₇BrF₃NO₃: 431.03 m/z, found 432.05, 434.05 [M+H, M+H+2]⁺.

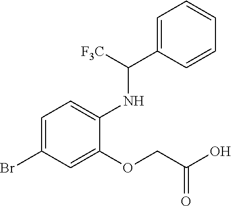 [SMILES: O=C(O)COc1cc(Br)ccc1NC(c1ccccc1)C(F)(F)F]

### 2-(5-bromo-2-((2,2,2-trifluoro-1-phenylethyl)amino)phenoxy)acetic acid

To a solution of ethyl 2-{5-bromo-2-[(2,2,2-trifluoro-1-phenylethyl)amino]phenoxy}acetate (240 mg, 0.555 mmol, 1 equiv) in THF/EtOH/H₂O (1:1:1, 15 mL) was added LiOH (66.49 mg, 2.775 mmol, 5 equiv). The reaction was stirred at rt for 1 h. The mixture was acidified to pH=5 with 1N HCl. Diluted with water (20 mL) and extracted with EA (3×20 mL). The combined extracts were washed with water, brine, dried over Na₂SO₄, filtered and concentrated. This resulted in 5-bromo-2-[(2,2,2-trifluoro-1-phenylethyl)amino]phenoxyacetic acid (200 mg, 89.12%) as a white oil. MS (ESI): mass calcd. for C₁₆H₁₃BrF₃NO₃: 403.00 m/z, found: 404.00, 406.00 [M+H, M+H+2]⁺.

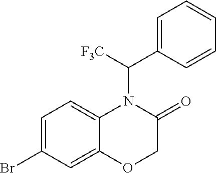 [SMILES: O=C1COc2cc(Br)ccc2N1C(c1ccccc1)C(F)(F)F]

### 7-bromo-4-(2,2,2-trifluoro-1-phenylethyl)-2H-benzo[b][1,4]oxazin-3(4H)-one

To a solution of 5-bromo-2-[(2,2,2-trifluoro-1-phenylethyl)amino]phenoxyacetic acid (200 mg, 0.495 mmol, 1 equiv) in EA (10 mL) was added T₃P (3 mL, 50% in EA). The reaction was stirred at rt for 1 h. Quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give 7-bromo-4-(2,2,2-trifluoro-1-phenylethyl)-2H-1,4-benzoxazin-3-one (140 mg, 73.27%) as a white solid. MS (ESI): mass calcd. for C₁₆H₁₁BrF₃NO₂: 384.99 m/z, found 385.90, 387.90 [M+H, M+H+2]⁺.

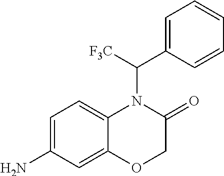 [SMILES: Nc1ccc2c(c1)OCC(=O)N2C(c1ccccc1)C(F)(F)F]

### 7-amino-4-(2,2,2-trifluoro-1-phenylethyl)-2H-benzo[b][1,4]oxazin-3(4H)-one

To a solution of 7-bromo-4-(2,2,2-trifluoro-1-phenylethyl)-2H-1,4-benzoxazin-3-one (140 mg, 0.363 mmol, 1 equiv) in EtOH/H₂O (4:1, 10 mL) was added (1R,2R)—N1,N2-dimethylcyclohexane-1,2-diamine (30.94 mg, 0.218 mmol, 0.6 equiv), CuSO₄ (57.86 mg, 0.363 mmol, 1 equiv), azidosodium (70.71 mg, 1.089 mmol, 3 equiv) and sodium ascorbate (144.37 mg, 0.726 mmol, 2 equiv). The reaction was stirred at 80° C. under N₂ for 3 h. After cooling down to rt, the reaction was quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 7-amino-4-(2,2,2-trifluoro-1-phenylethyl)-2H-1,4-benzoxazin-3-one (70 mg, 59.91%) as a yellow solid. MS (ESI): mass calcd. for C₁₆H₁₃F₃N₂O₂: 322.09 m/z, found 323.05 [M+H]⁺.

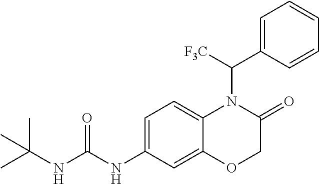 [SMILES: CC(C)(C)NC(=O)Nc1ccc2c(c1)OCC(=O)N2C(c1ccccc1)C(F)(F)F]

### 1-(tert-butyl)-3-(3-oxo-4-(2,2,2-trifluoro-1-phenylethyl)-3,4-dihydro-2H-benzo[b][1,4]oxazin-7-yl)urea

To a solution of 7-amino-4-(2,2,2-trifluoro-1-phenylethyl)-2H-1,4-benzoxazin-3-one (90 mg, 0.279 mmol, 1 equiv) in DCM/Toluene (2:1, 7.5 mL) was added 2-isocyanato-2-methylpropane (166.10 mg, 1.674 mmol, 6 equiv). The reaction was stirred at 90° C. under N₂ for 3 h. After cooling down to rt, the reaction was quenched with water (10 mL) and extracted with DCM (3×10 mL). The combined extracts were washed with water, brine, dried over Na₂SO₄, filtered and concentrated. The residue was purified by preparative HPLC using a XBridge Shield RP18 OBD Column, 30*150 mm, 5 m column (eluent: 45% to 67% (v/v) CH₃CN and Water (10 mmol/L NH₄HCO₃)) to afford the title compound 3-tert-butyl-1-[3-oxo-4-(2,2,2-trifluoro-1-phenylethyl)-2H-1,4-benzoxazin-7-yl]urea (22.3 mg, 18.95%) as a white solid. MS (ESI): mass calcd. for C₂₁H₂₂F₃N₃O₃: 421.16 m/z, found 422.15 [M+H]⁺. ¹H NMR (400 MHz, DMSO-d₆) δ 8.03-8.30 (m, 1H), 7.37-7.60 (m, 5H), 7.15-7.30 (m, 1H), 6.78-6.96 (m, 1H), 6.56-6.78 (m, 2H), 5.80-6.06 (m, 1H), 4.68-4.88 (m, 2H), 1.20-1.39 (m, 9H). ¹⁹F NMR (376 MHz, DMSO) δ(ppm): −63.7904.

### Example 297: (S)-1-(tert-butyl)-3-(1-(1-(3-methoxyphenyl)ethyl)-2-oxo-1,2,3,4-tetrahydroquinolin-6-yl)urea

### Synthetic Scheme

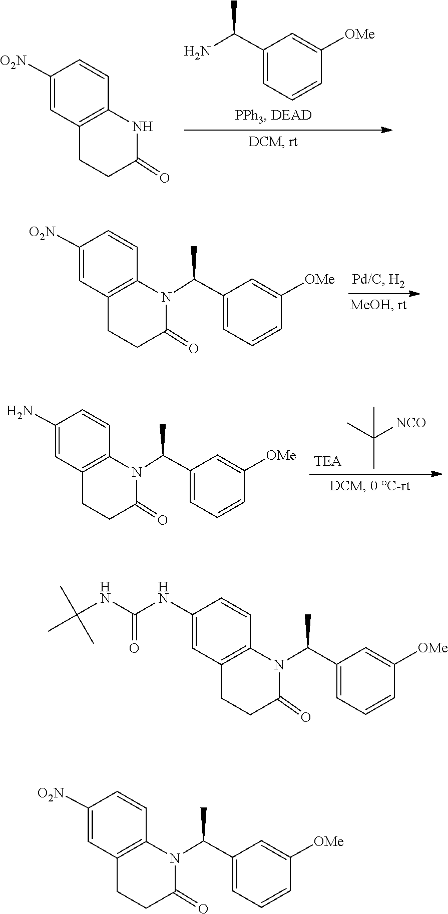

### (S)-1-(1-(3-methoxyphenyl)ethyl)-6-nitro-3,4-dihydroquinolin-2(1H)-one

To a solution of (1R)-1-(3-methoxyphenyl)ethanol (238 mg, 1.561 mmol, 1 equiv) in DCM (6 mL) was added 6-nitro-3,4-dihydro-1H-quinolin-2-one (300 mg, 1.561 mmol, 1 equiv), triphenylphosphine (409 mg, 1.561 mmol, 1 equiv) and DEAD (272 mg, 1.561 mmol, 1 equiv) at 0° C. under a nitrogen atmosphere. The resulting mixture was stirred overnight at rt. The reaction mixture was quenched by water (10 mL) and extracted with EA (3×10 mL). The combined organic extracts were washed with brine (10 mL), dried over anhydrous Na₂SO₄ and concentrated under vacuum. The crude product was purified by flash chromatography with PE/EA (3:1) to afford (S)-1-(1-(3-methoxyphenyl)ethyl)-6-nitro-3,4-dihydroquinolin-2(1H)-one (160 mg, 21.86%) as a yellow solid. MS (ESI): mass calcd. for C₁₈H₁₈N₂O₄, 326.13 m/z, found 327.10 [M+H]⁺.

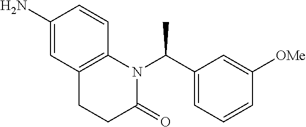 [SMILES: COc1cccc([C@H](C)N2C(=O)CCc3cc(N)ccc32)c1]

### (S)-6-amino-1-(1-(3-methoxyphenyl)ethyl)-3,4-dihydroquinolin-2(1H)-one

To a mixture of (S)-1-(1-(3-methoxyphenyl)ethyl)-6-nitro-3,4-dihydroquinolin-2(1H)-one (160 mg, 0.490 mmol, 1 equiv) in methanol (20 mL) was added Pd/C (10%) under nitrogen atmosphere. The mixture was hydrogenated at room temperature for 1 h under hydrogen atmosphere using a hydrogen balloon, filtered through a Celite pad and concentrated under reduced pressure. This resulted in (S)-6-amino-1-(1-(3-methoxyphenyl)ethyl)-3,4-dihydroquinolin-2(1H)-one (100 mg, 68.82%) as a white solid. MS (ESI): mass calcd. for C₁₈H₂₀N₂O₂, 296.15 m/z, found 297.15 [M+H]⁺.

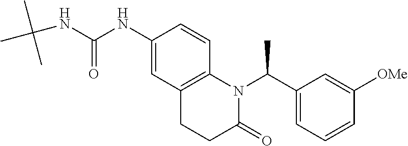 [SMILES: COc1cccc([C@H](C)N2C(=O)CCc3cc(NC(=O)NC(C)(C)C)ccc32)c1]

### (S)-1-(tert-butyl)-3-(1-(1-(3-methoxyphenyl)ethyl)-2-oxo-1,2,3,4-tetrahydroquinolin-6-yl)urea

To a mixture of (S)-6-amino-1-(1-(3-methoxyphenyl)ethyl)-3,4-dihydroquinolin-2(1H)-one (100 mg, 0.337 mmol, 1 equiv) and TEA (68 mg, 0.674 mmol, 2 equiv) in DCM (5 mL) was added 2-isocyanato-2-methylpropane (100 mg, 1.011 mmol, 3 equiv) at 0° C. The reaction was stirred for overnight at rt. The reaction mixture was quenched by water (10 mL) and extracted with DCM (3×10 mL). The organic layers were combined, dried over anhydrous sodium sulfate, filtered and concentrated under vacuum. The crude was purified by flash chromatography with PE/EA (1:1) and Prep-HPLC (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 37% B to 59% B in 10 min, 59% B; Wave Length: 254/220 nm) to give (S)-1-(tert-butyl)-3-(1-(1-(3-methoxyphenyl)ethyl)-2-oxo-1,2,3,4-tetrahydroquinolin-6-yl)urea (31.1 mg, 23.24%) as a white solid. MS (ESI): mass calcd. for C₂₃H₂₉N₃O₃, 395.22 m/z, found 396.15 [M+H]⁺. ¹H NMR (300 MHz, DMSO-d₆) δ 8.06 (s, 1H), 7.32 (d, J=2.4 Hz, 1H), 7.25 (t, J=7.9 Hz, 1H), 6.88-6.74 (m, 4H), 6.49 (d, J=8.8 Hz, 1H), 6.12 (q, J=7.1 Hz, 1H), 5.91 (s, 1H), 3.72 (s, 3H), 2.76-2.84 (m, 2H), 2.56-2.63 (m, 2H), 1.66 (d, J=7.1 Hz, 3H), 1.25 (s, 9H).

### Example 298: 1-(tert-butyl)-3-(1-(chroman-4-yl)-2-oxo-1,2,3,4-tetrahydroquinolin-6-yl)urea

### Synthetic Scheme

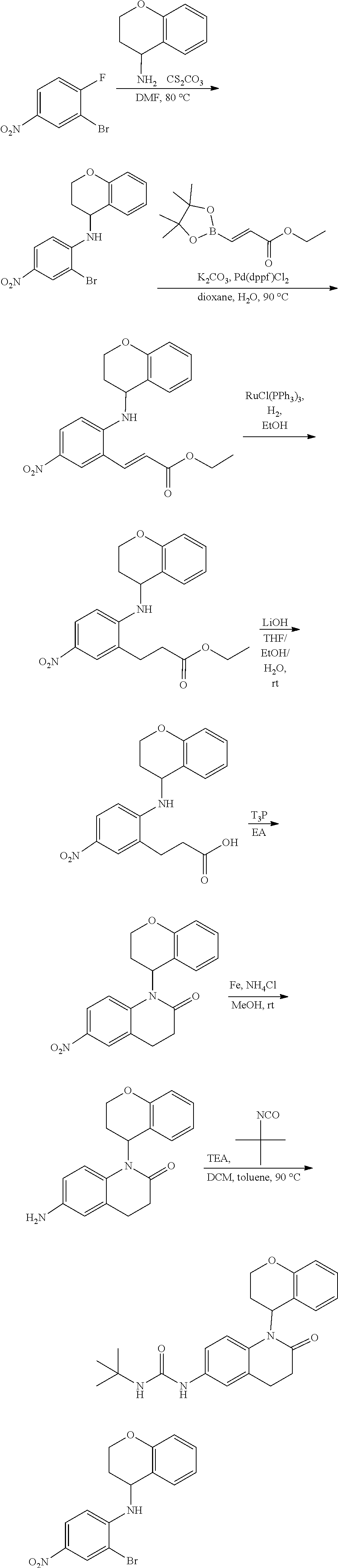

### N-(2-bromo-4-nitrophenyl)chroman-4-amine

To a stirred mixture of 2-bromo-1-fluoro-4-nitrobenzene (1.5 g, 6.818 mmol, 1 equiv) and 3,4-dihydro-2H-1-benzopyran-4-amine (1.02 g, 6.818 mmol, 1 equiv) in DMF (20 mL) was added Cs₂CO₃ (4.44 g, 13.636 mmol, 2 equiv) at 25° C. under air atmosphere. The resulting mixture was stirred for 2 h at room temperature. Desired product could be detected by LCMS. The mixture was diluted with water (50 mL). The resulting mixture was extracted with EA (3×50 mL). The combined organic layers were dried over anhydrous Na₂SO₄. After filtration, the filtrate was concentrated under reduced pressure. The residue obtained was purified by silica gel chromatography (0-100% ethyl acetate/petroleum ether) to afford N-(2-bromo-4-nitrophenyl)chroman-4-amine (900 mg, 37.8%) as a white solid. MS (ESI): mass calcd. for C₁₅H₁₃BrN₂O₃: 348.00 m/z, found 349.00 [M+H]⁺.

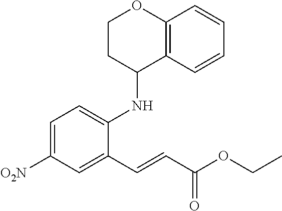 [SMILES: CCOC(=O)/C=C/c1cc([N+](=O)[O-])ccc1NC1CCOc2ccccc21]

### ethyl (E)-3-(2-(chroman-4-ylamino)-5-nitrophenyl)acrylate

To a stirred mixture of N-(2-bromo-4-nitrophenyl)chroman-4-amine (465 mg, 1.332 mmol, 1 equiv) and ethyl (2E)-3-(4,4,5,5-tetramethyl-1,3,2-dioxaborolan-2-yl)prop-2-enoate (451.60 mg, 1.998 mmol, 1.5 equiv) in dioxane (15 mL) and H₂O (1.5 mL) was added K₂CO₃ (552.13 mg, 3.996 mmol, 3 equiv) and Pd(dppf)Cl₂ (97.44 mg, 0.133 mmol, 0.1 equiv) at 25° C. under air atmosphere. The resulting mixture was stirred for 2 h at 90 degrees C. under nitrogen. Desired product could be detected by LCMS. The mixture was diluted with water (30 mL). The resulting mixture was extracted with EA (3×30 mL). The combined organic layers were dried over anhydrous Na₂SO₄. After filtration, the filtrate was concentrated under reduced pressure. The residue obtained was purified by silica gel chromatography (0-100% ethyl acetate/petroleum ether) to afford ethyl (E)-3-(2-(chroman-4-ylamino)-5-nitrophenyl)acrylate (480 mg, 97.84%) as a white solid. MS (ESI): mass calcd. for C₂₀H₂₀N₂O₅: 368.14 m/z, found 369.15 [M+H]⁺.

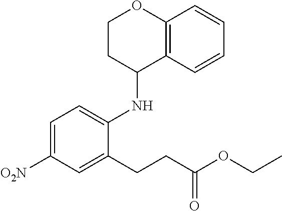 [SMILES: CCOC(=O)CCc1cc([N+](=O)[O-])ccc1NC1CCOc2ccccc21]

### ethyl 3-(2-(chroman-4-ylamino)-5-nitrophenyl)propanoate

To a stirred mixture of ethyl (E)-3-(2-(chroman-4-ylamino)-5-nitrophenyl)acrylate (400 mg, 1.086 mmol, 1 equiv) in EtOH (5 mL) was added RuCl(PPh₃)₂ (399.05 mg, 0.434 mmol, 0.40 equiv) at 25 degrees C. under air atmosphere. The resulting mixture was stirred for 2 h at room temperature under hydrogen. Desired product could be detected by LCMS. The resulting mixture was filtered, and the filter cake was washed with EtOH (4×10 mL). The filtrate was concentrated under reduced pressure. The residue obtained was purified by silica gel chromatography (0-100% ethyl acetate/petroleum ether) to afford ethyl 3-(2-(chroman-4-ylamino)-5-nitrophenyl)propanoate (190 mg, 47.24%) as a white solid. MS (ESI): mass calcd. for C₃₀H₄₂N₄O₄: 370.15 m/z, found 371.15 [M+H]⁺.

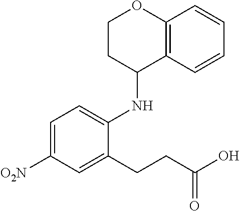 [SMILES: O=C(O)CCc1cc([N+](=O)[O-])ccc1NC1CCOc2ccccc21]

### 3-(2-(chroman-4-ylamino)-5-nitrophenyl)propanoic acid

To a stirred mixture of ethyl 3-(2-(chroman-4-ylamino)-5-nitrophenyl)propanoate (160 mg, 0.432 mmol, 1 equiv) in H₂O (1 mL), EtOH (1 mL) and THF (1 mL) was added LiOH (31.04 mg, 1.296 mmol, 3 equiv) at 0 degrees C. under air atmosphere. The resulting mixture was stirred for 2 h at room temperature. Desired product could be detected by LCMS. The mixture was basified to PH=5 with 2N HCl and diluted with water (20 mL). The resulting mixture was extracted with EA (3×20 mL). The combined organic layers were dried over anhydrous Na₂SO₄. After filtration, the filtrate was concentrated under reduced pressure to afford 3-(2-(chroman-4-ylamino)-5-nitrophenyl)propanoic acid (120 mg, 81.15%) as a white solid. MS (ESI): mass calcd. for C₁₈H₁₈N₂O₅: 342.12 m/z, found 341.00 [M−H]⁻.

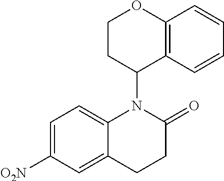 [SMILES: O=C1CCc2cc([N+](=O)[O-])ccc2N1C1CCOc2ccccc21]

### 1-(chroman-4-yl)-6-nitro-3,4-dihydroquinolin-2(1H)-one

The 3-(2-(chroman-4-ylamino)-5-nitrophenyl)propanoic acid (110 mg, 0.321 mmol, 1 equiv) was dissolved in T₃P (2 mL) and EA (2 mL) at 0 degrees C. under air atmosphere. The resulting mixture was stirred for 2 h at room temperature. Desired product could be detected by LCMS. The mixture was diluted with water (10 mL). The resulting mixture was extracted with EA (3×10 mL). The organic layers were combined, dried over anhydrous Na₂SO₄, filtered and concentrated. The residue obtained was purified by silica gel chromatography (0-100% ethyl acetate/petroleum ether) to afford 1-(chroman-4-yl)-6-nitro-3,4-dihydroquinolin-2(1H)-one (100 mg, 95.96%) as a white solid. MS (ESI): mass calcd. for C₁₈H₁₆N₂O₄: 324.11 m/z, found 325.05[M+H]⁺.

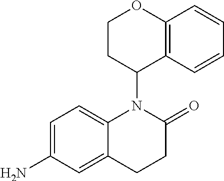 [SMILES: Nc1ccc2c(c1)CCC(=O)N2C1CCOc2ccccc21]

### 6-amino-1-(chroman-4-yl)-3,4-dihydroquinolin-2(1H)-one

To a stirred mixture of 1-(chroman-4-yl)-6-nitro-3,4-dihydroquinolin-2(1H)-one (100 mg, 0.308 mmol, 1 equiv) in MeOH (5 mL) was added Fe (172.18 mg, 3.080 mmol, 10 equiv) and NH₄C₁ (164.92 mg, 3.080 mmol, 10 equiv) at room temperature under air atmosphere. The resulting mixture was stirred for 2 h at 80 degrees C. Desired product could be detected by LCMS. After cooling down to room temperature, the mixture was diluted with water (10 mL). The resulting mixture was extracted with EA (3×10 mL). The organic layers were combined, dried over anhydrous Na₂SO₄, filtered and concentrated. The residue obtained was purified by silica gel chromatography (0-100% ethyl acetate/petroleum ether) to afford the 6-amino-1-(chroman-4-yl)-3,4-dihydroquinolin-2(1H)-one (50 mg, 55.09%) as a white solid. MS (ESI): mass calcd. for C₁₈H₁₈N₂O₂: 294.14 m/z, found 295.05[M+H]⁺.

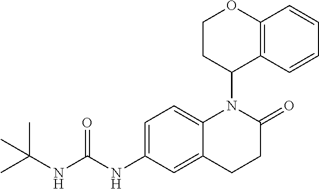 [SMILES: CC(C)(C)NC(=O)Nc1ccc2c(c1)CCC(=O)N2C1CCOc2ccccc21]

### 1-(tert-butyl)-3-(1-(chroman-4-yl)-2-oxo-1,2,3,4-tetrahydroquinolin-6-yl)urea

To a stirred mixture of 6-amino-1-(chroman-4-yl)-3,4-dihydroquinolin-2(1H)-one (50 mg, 0.170 mmol, 1 equiv) and 2-isocyanato-2-methylpropane (84.20 mg, 0.850 mmol, 5 equiv) in DCM (2 mL) and toluene (0.5 mL) was added TEA (85.94 mg, 0.850 mmol, 5 equiv) dropwise at room temperature under air atmosphere. The resulting mixture was stirred for 2 h at 90 degrees C. Desired product could be detected by LCMS. After cooling down to room temperature, the mixture was diluted with water (10 mL). The resulting mixture was extracted with EA (3×10 mL). The organic layers were combined, dried over anhydrous Na₂SO₄, filtered and concentrated. The residue was purified by preparative HPLC using a X Bridge Prep OBD C18 150 mm×30 mm×5 μm column (eluent: 26% to 42% (v/v) CH₃CN and H₂O with 10 mmol/L NH₄HCO₃) to afford the 1-(tert-butyl)-3-(1-(chroman-4-yl)-2-oxo-1,2,3,4-tetrahydroquinolin-6-yl)urea (7.2 mg, 10.72%) as a white solid. MS (ESI): mass calcd. for C₂₃H₂₇N₃O₃: 393.21 m/z, found 394.05[M+H]⁺. ¹H-NMR (300 MHz, DMSO-d₆) δ 8.07 (s, 1H), 7.34 (d, J=2.5 Hz, 1H), 7.04-7.15 (m, 1H), 6.71-6.90 (m, 4H), 6.08-6.68 (m, 2H), 5.92 (s, 1H), 4.39 (d, J=10.8 Hz, 1H), 4.19-4.33 (m, 1H), 2.78-2.89 (m, 2H), 2.58-2.69 (m, 3H), 1.92 (s, 1H), 1.24 (s, 9H).

### Example 299: (S)—N-(2-oxo-1-(1-(6-(trifluoromethyl)pyridin-2-yl)ethyl)-1,2-dihydroquinoxalin-6-yl)-3-(3-(trifluoromethyl)phenyl)propanamide

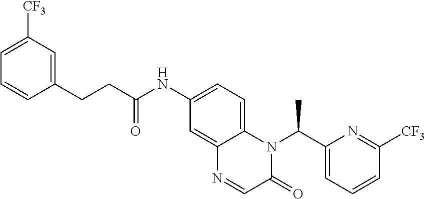 [SMILES: C[C@@H](c1cccc(C(F)(F)F)n1)n1c(=O)cnc2cc(NC(=O)CCc3cccc(C(F)(F)F)c3)ccc21]

### (S)—N-(2-oxo-1-(1-(6-(trifluoromethyl)pyridin-2-yl)ethyl)-1,2-dihydroquinoxalin-6-yl)-3-(3-(trifluoromethyl)phenyl)propanamide

To a solution of 3-[3-(trifluoromethyl)phenyl]propanoic acid (43.07 mg, 0.197 mmol, 1.1 equiv) in DMF (5 mL) was added 6-amino-1-[(1S)-1-[6-(trifluoromethyl)pyridin-2-yl]ethyl]quinoxalin-2-one (60 mg, 0.179 mmol, 1 equiv), HATU (88.72 mg, 0.233 mmol, 1.3 equiv) and DIEA (69.59 mg, 0.537 mmol, 3 equiv) at 0° C. The reaction was stirred at rt for 1 h. Quenched with water (20 mL) and extracted with EA (3×20 mL). The combined extracts were washed with water, brine, dried over Na₂SO₄, filtered and concentrated. The residue was purified by preparative HPLC using a XBridge Prep OBD C18 Column, 30*150 mm, 5 μm column (eluent: 17% to 42% (v/v) CH₃CN and H₂O with 10 mmol/L NH₄HCO₃) to afford the title compound N-{2-oxo-1-[(1S)-1-[6-(trifluoromethyl)pyridin-2-yl]ethyl]quinoxalin-6-yl}-3-[3-(trifluoromethyl)phenyl]propanamide (28.4 mg, 29.61%) as a yellow solid. MS (ESI): mass calcd. for C₂₆H₂₀F₆N₄O₂: 534.15 m/z, found: 535.15 [M+H]⁺. ¹H NMR (400 MHz, DMSO-d₆) δ 10.15 (s, 1H), 8.25 (s, 1H), 8.10-8.20 (m, 1H), 7.95-8.10 (m, 1H), 7.77-7.86 (m, 1H), 7.50-7.70 (m, 6H), 7.00-7.50 (m, 1H), 6.55 (s, 1H), 2.97-3.11 (m, 2H), 2.62-2.77 (m, 2H), 1.90-1.98 (m, 3H). ¹⁹F NMR (376 MHz, DMSO) δ(ppm): −60.9926, −66.5643.

### Example 300: (S)-4,4-dimethyl-N-(2-oxo-1-(1-(6-(trifluoromethyl)pyridin-2-yl)ethyl)-1,2-dihydroquinoxalin-6-yl)pentanamide

### Synthetic Scheme

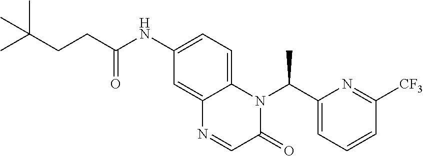 [SMILES: C[C@@H](c1cccc(C(F)(F)F)n1)n1c(=O)cnc2cc(NC(=O)CCC(C)(C)C)ccc21]

### (S)-4,4-dimethyl-N-(2-oxo-1-(1-(6-(trifluoromethyl)pyridin-2-yl)ethyl)-1,2-dihydroquinoxalin-6-yl)pentanamide

To a solution of 4,4-dimethyl valeric acid (25.70 mg, 0.197 mmol, 1.1 equiv) in DMF (5 mL) was added 6-amino-1-[(1S)-1-[6-(trifluoromethyl)pyridin-2-yl]ethyl]quinoxalin-2-one (60 mg, 0.179 mmol, 1 equiv), HATU (88.72 mg, 0.233 mmol, 1.3 equiv) and DIEA (69.59 mg, 0.537 mmol, 3 equiv) at 0° C. The reaction was stirred at rt for 1 h. Quenched with water (20 mL) and extracted with EA (3×20 mL). The combined extracts were washed with water, brine, dried over Na₂SO₄, filtered and concentrated. The residue was purified by preparative HPLC using a XSelect CSH Fluoro Phenyl, 30*150 mm, 5 m column (eluent: 33% to 56% (v/v) CH₃CN and H₂O with 10 mmol/L NH₄HCO₃) to afford the title compound 4,4-dimethyl-N-{2-oxo-1-[(1S)-1-[6-(trifluoromethyl)pyridin-2-yl]ethyl]quinoxalin-6-yl}pentanamide (31.9 mg, 39.81%) as a yellow solid. MS (ESI): mass calcd. for C₂₃H₂₅F₃N₄O₂: 446.19 m/z, found: 447.20 [M+H]⁺. ¹H NMR (400 MHz, DMSO-d₆) δ 10.12 (s, 1H), 8.12-8.30 (m, 2H), 7.93-8.10 (m, 1H), 7.70-7.85 (m, 1H), 7.50-7.70 (m, 2H), 6.98-7.45 (m, 1H), 6.30-6.64 (m, 1H), 2.15-2.37 (m, 2H), 1.81-2.00 (m, 3H), 1.40-1.60 (m, 2H), 0.90 (s, 9H). ¹⁹F NMR (376 MHz, DMSO) δ(ppm): −66.5505.

### Example 301: 1-(1-benzyl-2-oxo-3-(trifluoromethyl)-1,2,3,4-tetrahydroquinolin-6-yl)-3-(tert-butyl)urea

### Synthetic Scheme

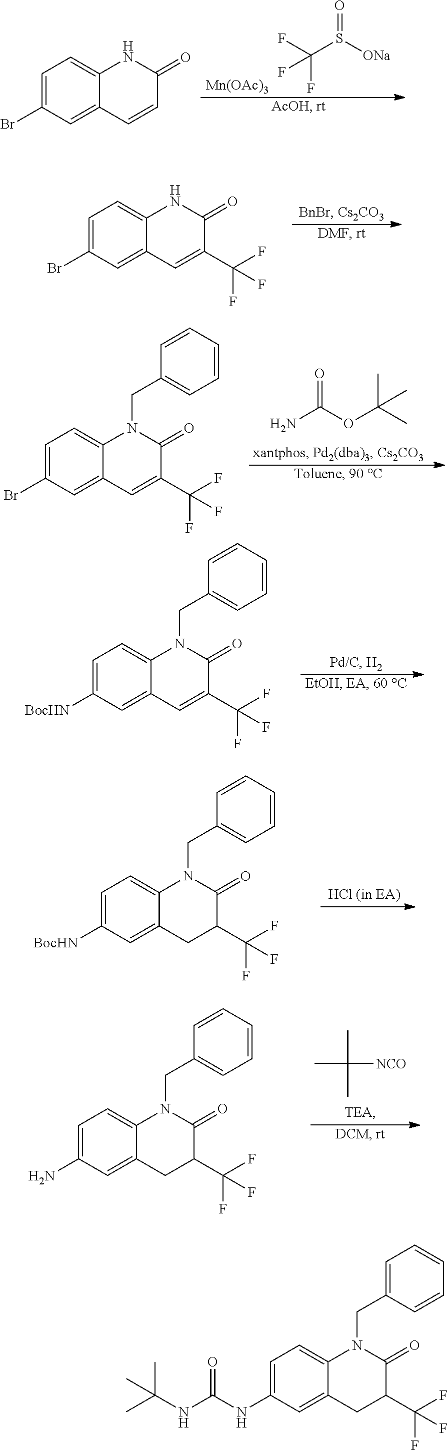

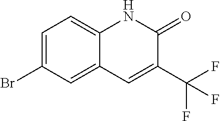 [SMILES: O=c1[nH]c2ccc(Br)cc2cc1C(F)(F)F]

### 6-bromo-3-(trifluoromethyl)quinolin-2(1H)-one

6-bromoquinolin-2(1H)-one (1.0 g, 4.46 mmol, 1.0 equiv), Mn(OAc)₃·2H₂O (3.59 g, 17.84 mmol, 4.0 equiv) and sodium trifluoromethanesulfinate (1.57 g, 13.87 mmol, 3.0 equiv) were dissolved in glacial acetic acid (25 mL), and the reaction was stirred at 25° C. for 24 hours. Water (100 mL) was added to the system, the mixture was extracted with ethyl acetate (100 mL×2), the organic phases were combined, washed with saturated saline (100 mL), dried over anhydrous sodium sulfate, filtered, the filtrate was concentrated under reduced pressure, and the crude product was purified by silica gel column chromatography (PE/EA=3:1) to obtain 6-bromo-3-(trifluoromethyl)quinolin-2(1H)-one (0.5 g, 38.46%) as a yellow solid. MS (ESI): mass calcd. for C₁₀H₅BrF₃NO, 290.95 m/z, found 292.15 [M+H]⁺.

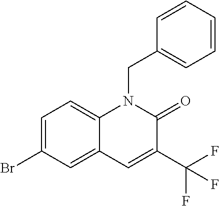 [SMILES: O=c1c(C(F)(F)F)cc2cc(Br)ccc2n1Cc1ccccc1]

### 1-benzyl-6-bromo-3-(trifluoromethyl)quinolin-2(1H)-one

A solution of 6-bromo-3-(trifluoromethyl)quinolin-2(1H)-one (480 mg, 1.64 mmol, 1 equiv) in DMF (5 mL) was added BnBr (0.8 g, 4.90 mmol, 3.0 equiv) and Cs₂CO₃ (2.1 g, 6.560 mmol, 4.0 equiv) at rt. The resulting mixture was stirred for 2 h at rt. The reaction was quenched with water (20 mL). The resulting mixture was extracted with EA (3×30 mL). The combined organic layers were washed with NaCl (aq., 3×10 mL), dried over anhydrous Na₂SO₄. After filtration, the filtrate was concentrated under reduced pressure. The residue was purified by silica gel column chromatography, eluted with PE/EA (3/1) to afford 1-benzyl-6-bromo-3-(trifluoromethyl)quinolin-2(1H)-one (400 mg, 63.89%%) as a white solid. MS (ESI): mass calcd. for C₁₇H₁₁BrF₃NO, 381.00 m/z, found 382.15 [M+H]⁺.

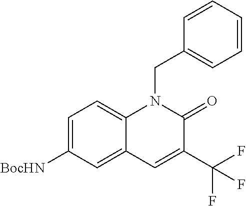 [SMILES: CC(C)(C)OC(=O)Nc1ccc2c(c1)cc(C(F)(F)F)c(=O)n2Cc1ccccc1]

### tert-butyl (1-benzyl-2-oxo-3-(trifluoromethyl)-1,2-dihydroquinolin-6-yl)carbamate

A solution of 1-benzyl-6-bromo-3-(trifluoromethyl)quinolin-2(1H)-one (0.2 g, 0.524 mmol, 1 equiv) in toluene (5 mL) was treated with tert-butyl carbamate (123 mg, 1.04 mmol, 2 equiv), Cs₂CO₃ (0.5 g, 1.57 mmol, 3 equiv), XantPhos (30 mg, 0.052 mmol, 0.1 equiv) and Pd₂(dba)₃ (48 mg, 0.052 mmol, 0.1 equiv) for overnight at 90° C. under nitrogen atmosphere. The reaction mixture was quenched by water (10 mL) and extracted with EA (3×10 mL). The combined organic extracts were washed with brine (10 mL), dried over anhydrous Na₂SO₄ and concentrated under vacuum. The crude product was purified by flash chromatography with PE/EA (3:1) to afford tert-butyl (1-benzyl-2-oxo-3-(trifluoromethyl)-1,2-dihydroquinolin-6-yl)carbamate (120 mg, 54.79%). MS (ESI): mass calcd. for C₂₂H₂₁F₃N₂O₃, 418.15.00 m/z, found 419.10 [M+H]⁺.

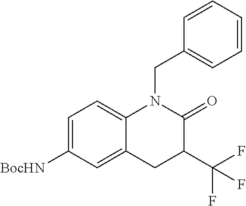 [SMILES: CC(C)(C)OC(=O)Nc1ccc2c(c1)CC(C(F)(F)F)C(=O)N2Cc1ccccc1]

### tert-butyl (1-benzyl-2-oxo-3-(trifluoromethyl)-1,2,3,4-tetrahydroquinolin-6-yl)carbamate

To a solution of tert-butyl (1-benzyl-2-oxo-3-(trifluoromethyl)-1,2-dihydroquinolin-6-yl)carbamate (90 mg, 0.215 mmol, 1 equiv) in EtOH (5 mL) and HOAc (1 mL) was added Pd/C (50 mg) under nitrogen atmosphere. The resulting mixture was placed at 60° C. under a hydrogen atmosphere (50 bar) until the starting material was totally consumed by LCMS. The mixture was filtered and concentrated under vacuum to afford tert-butyl (1-benzyl-2-oxo-3-(trifluoromethyl)-1,2,3,4-tetrahydroquinolin-6-yl)carbamate (90 mg crude) as a white solid.

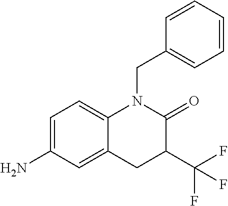 [SMILES: Nc1ccc2c(c1)CC(C(F)(F)F)C(=O)N2Cc1ccccc1]

### 6-amino-1-benzyl-3-(trifluoromethyl)-3,4-dihydroquinolin-2(1H)-one

To a stirred solution of tert-butyl (1-benzyl-2-oxo-3-(trifluoromethyl)-1,2,3,4-tetrahydroquinolin-6-yl)carbamate (90 mg crude) in HCl (4N in EA, 3 mL) at rt. The reaction was stirred for 2 h at rt. The resulting mixture was concentrated under reduced pressure to afford 6-amino-1-benzyl-3-(trifluoromethyl)-3,4-dihydroquinolin-2(1H)-one (80 mg crude) as a white solid.

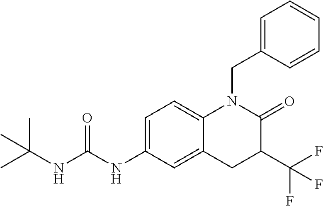 [SMILES: CC(C)(C)NC(=O)Nc1ccc2c(c1)CC(C(F)(F)F)C(=O)N2Cc1ccccc1]

### 1-(1-benzyl-2-oxo-3-(trifluoromethyl)-1,2,3,4-tetrahydroquinolin-6-yl)-3-(tert-butyl)urea

A solution of 6-amino-1-benzyl-3-(trifluoromethyl)-3,4-dihydroquinolin-2(1H)-one (80 mg crude), TEA (80 mg) and 2-isocyanato-2-methylpropane (80 mg) in DCM (5 mL) was stirred for overnight at rt. The mixture was concentrated under vacuum. The residue was purified by silica gel column chromatography, eluted with EA (46%-57%) and C18 column to afford 1-(1-benzyl-2-oxo-3-(trifluoromethyl)-1,2,3,4-tetrahydroquinolin-6-yl)-3-(tert-butyl)urea (2.7 mg) as a white solid. MS (ESI): mass calcd. for C₂₂H₂₄F₃N₃O₂: 419.18 m/z, found 420.10 [M+H]⁺. ¹H NMR (400 MHz, DMSO-d₆) δ 8.18 (s, 1H), 7.39 (d, J=2.5 Hz, 1H), 7.28-7.37 (m, 2H), 7.18-7.28 (m, 3H), 7.05 (dd, J=8.8, 2.5 Hz, 1H), 6.83 (d, J=8.8 Hz, 1H), 5.96 (s, 1H), 5.08-5.18 (m, 2H), 4.01-4.12 (m, 1H), 3.06-3.23 (m, 2H), 1.26 (s, 9H).

### Example 302: 1-(1-benzyl-2-oxo-1,2,3,4-tetrahydroquinazolin-6-yl)-3-(tert-butyl)urea

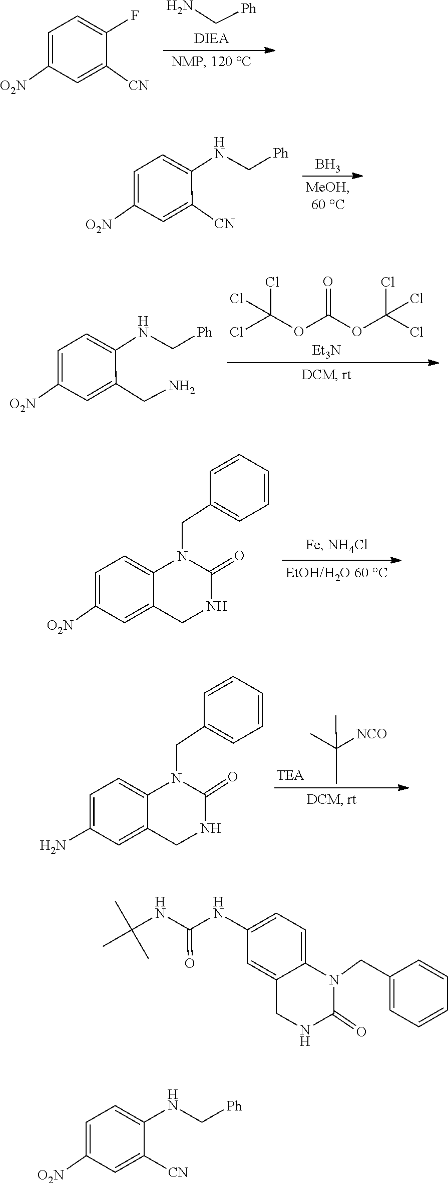

### 2-(benzylamino)-5-nitrobenzonitrile

A solution of 2-fluoro-5-nitrobenzonitrile (2.0 g, 12.040 mmol, 1 equiv) in NMP (20 mL) was added benzylamine (1.94 g, 18.060 mmol, 1.5 equiv) and DIEA (3.11 g, 24.080 mmol, 2.0 equiv). The resulting mixture was stirred for overnight at rt. The reaction mixture was quenched by water (10 mL) and extracted with EA (3×20 mL). The combined organic layers were washed with NaCl (3×20 mL), dried over anhydrous Na₂SO₄. After filtration, the filtrate was concentrated under reduced pressure. The residue was purified by silica gel column chromatography, eluted with PE/EA (3/1) to afford 2-(benzylamino)-5-nitrobenzonitrile (2.2 g, 72.15%) as an off-white solid. LC/MS: mass calcd. for C₁₄H₁₁N₃O₂: 253.09, found: 254.10 [M+H]⁺.

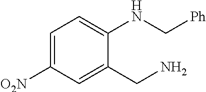 [SMILES: NCc1cc([N+](=O)[O-])ccc1NCc1ccccc1]

### 2-(aminomethyl)-N-benzyl-4-nitroaniline

A solution of 2-(benzylamino)-5-nitrobenzonitrile (500 mg, 1.974 mmol, 1 equiv) in methanol (5 mL) was added borane (54.61 mg, 3.948 mmol, 2.0 equiv) at 0° C. The resulting mixture was stirred for overnight at 60° C. The reaction mixture was quenched by water (5 mL) and extracted with EA (3×10 mL). The combined organic layers were washed with NaCl (3×10 mL), dried over anhydrous Na₂SO₄. After filtration, the filtrate was concentrated under reduced pressure. The residue was purified by silica gel column chromatography, eluted with PE/EA (3/1) to afford 2-(aminomethyl)-N-benzyl-4-nitroaniline (300 mg, 59.06%) as colorless oil. LC/MS: mass calcd. for C₁₄H₁₅N₃O₂: 257.12, found: 258.10[M+H]⁺.

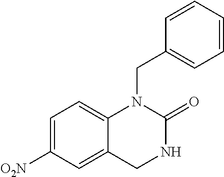 [SMILES: O=C1NCc2cc([N+](=O)[O-])ccc2N1Cc1ccccc1]

### 1-benzyl-6-nitro-3,4-dihydroquinazolin-2(1H)-one

A solution of 2-(aminomethyl)-N-benzyl-4-nitroaniline (130 mg, 0.505 mmol, 1 equiv) in DCM (3 mL) was added triphosgene (45.0 mg, 0.151 mmol, 0.3 equiv) and triethylamine (127.8 mg, 1.262 mmol, 2.5 equiv). The resulting mixture was stirred for overnight at rt. The reaction mixture was quenched by water (5 mL) and extracted with EA (3×10 mL). The combined organic layers were washed with NaCl (3×10 mL), dried over anhydrous Na₂SO₄. After filtration, the filtrate was concentrated under reduced pressure. The residue was purified by silica gel column chromatography, eluted with PE/EA (3/1) to afford 1-benzyl-6-nitro-3,4-dihydroquinazolin-2(1H)-one (37 mg, 25.85%) as a white solid. LC/MS: mass calcd. for C₁₅H₁₃N₃O₃: 283.10, found: 284.05 [M+H]⁺.

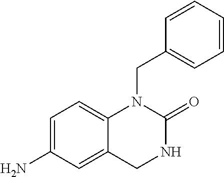 [SMILES: Nc1ccc2c(c1)CNC(=O)N2Cc1ccccc1]

### 6-amino-1-benzyl-3,4-dihydroquinazolin-2(1H)-one

A solution of 1-benzyl-6-nitro-3,4-dihydroquinazolin-2(1H)-one (32 mg, 0.113 mmol, 1 equiv) in EtOH (3 mL)/H₂O (0.3 mL) was added Fe (63.1 mg, 1.130 mmol, 10 equiv) and NH₄Cl (60.4 mg, 1.130 mmol, 10 equiv). The resulting mixture was stirred for 1 h at 60° C. The reaction mixture was quenched by water (5 mL) and extracted with EA (3×5 mL). The combined organic layers were washed with NaCl (3×5 mL), dried over anhydrous Na₂SO₄. After filtration, the filtrate was concentrated under reduced pressure. The residue was purified by silica gel column chromatography, eluted with PE/EA (1/1) to afford 6-amino-1-benzyl-3,4-dihydroquinazolin-2(1H)-one (20 mg, 69.90%) as an off-white solid. LC/MS: mass calcd. for C₁₅H₁₅N₃O: 253.12, found: 254.20 [M+H]⁺.

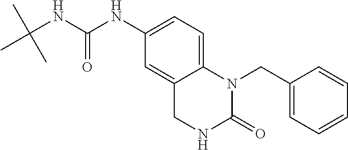 [SMILES: CC(C)(C)NC(=O)Nc1ccc2c(c1)CNC(=O)N2Cc1ccccc1]

### 1-(1-benzyl-2-oxo-1,2,3,4-tetrahydroquinazolin-6-yl)-3-(tert-butyl)urea

A solution of 6-amino-1-benzyl-3,4-dihydroquinazolin-2(1H)-one (20 mg, 0.079 mmol, 1 equiv) in DCM (26.8 mg, 0.316 mmol, 4.0 equiv) was added 2-isocyanato-2-methylpropane (23.5 mg, 0.237 mmol, 3.0 equiv) and TEA (3 mL) at rt. The resulting mixture was stirred for overnight at rt. The reaction mixture was quenched by water (5 mL) and extracted with EA (5 mL×3). The combined organic layers were washed with brine, dried over anhydrous Na₂SO₄. After filtration, the filtrate was concentrated under reduced pressure. The crude product 30 mg was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; Number Of Runs: 0) to afford 1-(1-benzyl-2-oxo-1,2,3,4-tetrahydroquinazolin-6-yl)-3-(tert-butyl)urea (3.0 mg, 10.77%) as a white solid. MS (ESI): mass calcd. C₂₀H₂₄N₄O₂ for: 352.19 m/z, found 353.10 [M+H]⁺. ¹H NMR (400 MHz, DMSO-d₆) δ 8.06 (s, 1H), 7.27-7.35 (m, 3H), 7.24-7.19 (m, 3H), 7.09 (s, 1H), 6.89 (dd, J=8.8, 2.5 Hz, 1H), 6.57 (d, J=8.8 Hz, 1H), 5.90 (s, 1H), 5.00 (s, 2H), 4.33 (s, 2H), 1.25 (s, 9H).

### Example 303: cyclopentyl ((R)-2-methyl-3-oxo-4-((S)-1-phenylethyl)-3,4-dihydro-2H-benzo[b][1,4]oxazin-7-yl)carbamate

### Synthetic Scheme

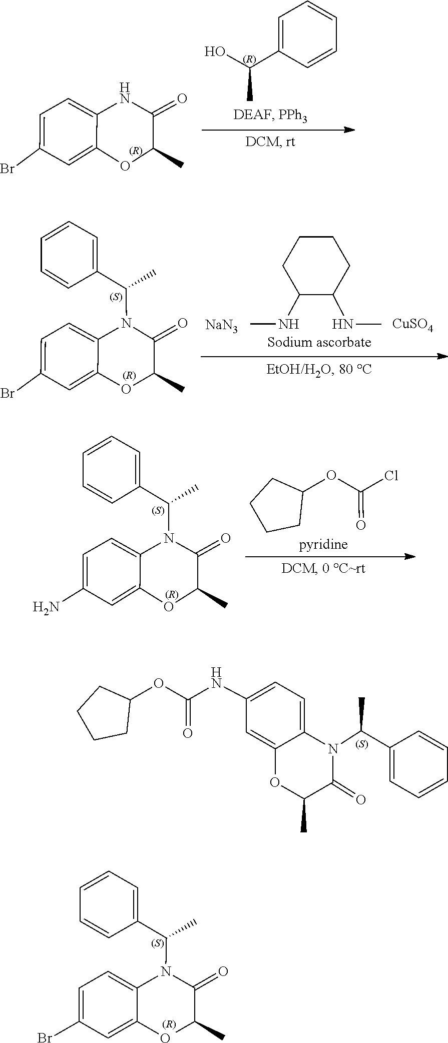

### (R)-7-bromo-2-methyl-4-((S)-1-phenylethyl)-2H-benzo[b][1,4]oxazin-3(4H)-one

To a stirred mixture of (R)-7-bromo-2-methyl-2H-benzo[b][1,4]oxazin-3(4H)-one (1 g, 4.131 mmol, 1 equiv) and (R)-1-phenylethan-1-ol (555.14 mg, 4.544 mmol, 1.1 equiv) in DCM (20 mL) was added PPh₃ (1.63 g, 6.197 mmol, 1.5 equiv) at 25° C. under air atmosphere. The resulting mixture was stirred at room temperature under nitrogen. After 30 min, the DEAD (719.44 mg, 4.131 mmol, 1 equiv) was added to the mixture. The resulting mixture was stirred for 2 h at room temperature under nitrogen. Desired product could be detected by LCMS. The mixture was diluted with water (50 mL). The resulting mixture was extracted with EA (3×50 mL). The combined organic layers were dried over anhydrous Na₂SO₄. After filtration, the filtrate was concentrated under reduced pressure. The residue obtained was purified by silica gel chromatography (0-50% ethyl acetate/petroleum ether) to afford the (R)-7-bromo-2-methyl-4-((S)-1-phenylethyl)-2H-benzo[b][1,4]oxazin-3(4H)-one (1 g, 69.92%) as a white solid. MS (ESI): mass calcd. for C₁₇H₁₆BrNO₂: 345.04 m/z, found 346.05 [M+H]⁺.

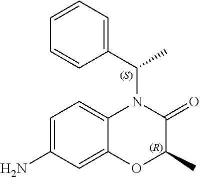

### (R)-7-amino-2-methyl-4-((S)-1-phenylethyl)-2H-benzo[b][1,4]oxazin-3(4H)-one

To a stirred mixture of (R)-7-bromo-2-methyl-4-((S)-1-phenylethyl)-2H-benzo[b][1,4]oxazin-3(4H)-one (1 g, 2.432 mmol, 1 equiv) in EtOH (8 mL) and H₂O (2 mL) was added NaN₃ (452.37 mg, 7.296 mmol, 3 equiv), N1,N2-dimethylcyclohexane-1,2-diamine (207.54 mg, 1.459 mmol, 0.6 equiv), CuSO₄ (388.10 mg, 2.432 mmol, 1 equiv) and sodium ascorbate (968.37 mg, 4.864 mmol, 2 equiv) at 25° C. under air atmosphere. The resulting mixture was stirred for 2 h at 80 degrees C. under nitrogen. Desired product could be detected by LCMS. After cooling down to room temperature, the mixture was diluted with water (30 mL). The resulting mixture was extracted with EA (3×30 mL). The combined organic layers were dried over anhydrous Na₂SO₄. After filtration, the filtrate was concentrated under reduced pressure. The residue obtained was purified by silica gel chromatography (0-100% ethyl acetate/petroleum ether) to afford the (R)-7-amino-2-methyl-4-((S)-1-phenylethyl)-2H-benzo[b][1,4]oxazin-3(4H)-one (300 mg, 43.70%) as a white solid. MS (ESI): mass calcd. for C₁₇H₁₈N₂O₂: 282.14 m/z, found 283.15 [M+H]⁺.

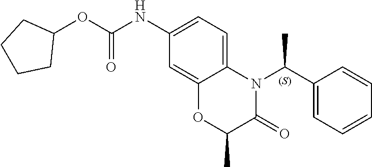

### cyclopentyl ((R)-2-methyl-3-oxo-4-((S)-1-phenylethyl)-3,4-dihydro-2H-benzo[b][1,4]oxazin-7-yl)carbamate

To a stirred mixture of (R)-7-amino-2-methyl-4-((S)-1-phenylethyl)-2H-benzo[b][1,4]oxazin-3(4H)-one (80 mg, 0.283 mmol, 1 equiv) and cyclopentyl carbonochloridate (126.31 mg, 0.849 mmol, 3 equiv) in DCM (3 mL) was added pyridine (67.24 mg, 0.849 mmol, 3 equiv) dropwise at 0 degrees C. under air atmosphere. The resulting mixture was stirred for 2 h at room temperature. Desired product could be detected by LCMS. The mixture was diluted with water (10 mL). The resulting mixture was extracted with EA (3×10 mL). The organic layers were combined, dried over anhydrous Na₂SO₄, filtered and concentrated. The residue was purified by preparative HPLC using a X Bridge Prep OBD C18 150 mm×30 mm×5 μm column (eluent: 51% to 73% (v/v) CH₃CN and H₂O with 10 mmol/L NH₄HCO₃) to afford the cyclopentyl ((R)-2-methyl-3-oxo-4-((S)-1-phenylethyl)-3,4-dihydro-2H-benzo[b][1,4]oxazin-7-yl)carbamate (14.3 mg, 12.73%) as a white solid. MS (ESI): mass calcd. for C₂₃H₂₆N₂O₄: 394.19 m/z, found 395.10 [M+H]⁺. ¹H-NMR (300 MHz, DMSO-d₆) δ 9.50 (s, 1H), 7.31-7.41 (m, 2H), 7.20-7.31 (m, 3H), 7.20 (d, J=2.4 Hz, 1H), 6.81-6.91 (m, 1H), 6.67 (d, J=8.9 Hz, 1H), 6.03-6.16 (m, 1H), 5.00-5.10 (m, 1H), 4.70-4.82 (m, 1H), 1.79-1.94 (m, 2H), 1.71-1.78 (m, 3H), 1.49-1.70 (m, 6H), 1.45 (d, J=6.8 Hz, 3H).

### Example 304: cyclopentyl N-[(2R)-2-methyl-3-oxo-4-{[6-(trifluoromethyl)pyridin-2-yl]methyl}-2H-1,4-benzoxazin-7-yl]carbamate

### Synthetic Scheme

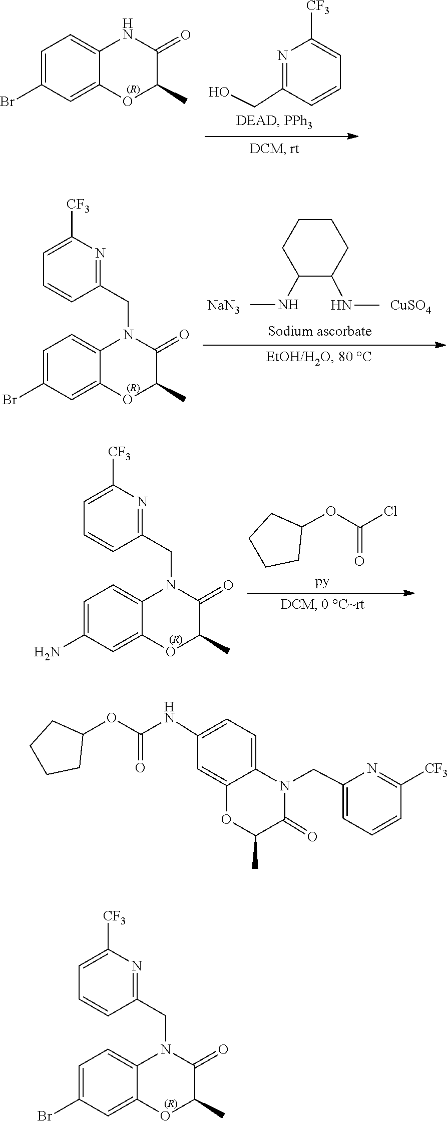

### (2R)-7-bromo-2-methyl-4-{[6-(trifluoromethyl)pyridin-2-yl]methyl}-2H-1,4-benzoxazin-3-one

To a solution of (2R)-7-bromo-2-methyl-2,4-dihydro-1,4-benzoxazin-3-one (500 mg, 2.066 mmol, 1 equiv) in DCM (10 mL) was added [6-(trifluoromethyl)pyridin-2-yl]methanol (439.02 mg, 2.479 mmol, 1.2 equiv) and PPh₃ (812.65 mg, 3.099 mmol, 1.5 equiv). After the mixture was stirred at rt for 1 h, then DEAD (539.58 mg, 3.099 mmol, 1.5 equiv) was added at 0° C. The reaction was stirred at 25° C. for 2 h. Quenched with water (50 mL) and extracted with DCM (3×50 mL). The combined extracts were washed with water, brine, dried over Na₂SO₄, filtered and concentrated. The residue was purified by column chromatography on silica gel with EA/PE (0-100%) to give (2R)-7-bromo-2-methyl-4-{[6-(trifluoromethyl)pyridin-2-yl]methyl}-2H-1,4-benzoxazin-3-one (410 mg, 49.48%) as a white solid. MS(ESI): mass calcd. for C₁₆H₁₂BrF₃N₂O₂:400.00 m/z, found: 401.00, 403.00 [M+H, M+H+2]⁺.

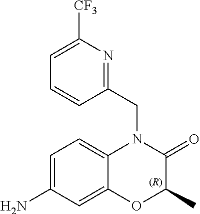

### (2R)-7-amino-2-methyl-4-{[6-(trifluoromethyl)pyridin-2-yl]methyl}-2H-1,4-benzoxazin-3-one

To a solution of (2R)-7-bromo-2-methyl-4-{[6-(trifluoromethyl)pyridin-2-yl]methyl}-2H-1,4-benzoxazin-3-one (600 mg, 1.287 mmol, 1 equiv) in EtOH (10 mL) and H₂O (1 mL) were added N1,N2-dimethylcyclohexane-1,2-diamine (109.84 mg, 0.772 mmol, 0.6 equiv) and CuSO₄ (205.41 mg, 1.287 mmol, 1 equiv), sodium ascorbate (512.53 mg, 2.574 mmol, 2 equiv). The reaction was stirred at 80° C. for 2 h. The mixture was allowed to cool down to room temperature. Quenched with water (50 mL) and extracted with DCM (3×50 mL). The combined extracts were washed with water, brine, dried over Na₂SO₄, filtered and concentrated. The residue was purified by column chromatography on silica gel with EA/PE (0-100%) to give (2R)-7-amino-2-methyl-4-{[6-(trifluoromethyl)pyridin-2-yl]methyl}-2H-1,4-benzoxazin-3-one (290 mg, 66.80%) as a white solid. MS (ESI): mass calcd. for C₁₆H₁₄F₂N₃O₂:337.10 m/z, found: 338.10 [M+H]⁺.

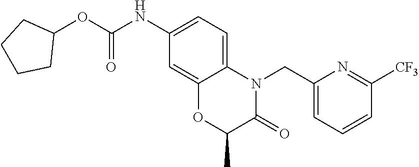 [SMILES: C[C@H]1Oc2cc(NC(=O)OC3CCCC3)ccc2N(Cc2cccc(C(F)(F)F)n2)C1=O]

### cyclopentyl N-[(2R)-2-methyl-3-oxo-4-{[6-(trifluoromethyl)pyridin-2-yl]methyl}-2H-1,4-benzoxazin-7-yl]carbamate

To a solution of (2R)-7-amino-2-methyl-4-{[6-(trifluoromethyl)pyridin-2-yl]methyl}-2H-1,4-benzoxazin-3-one (80 mg, 0.237 mmol, 1 equiv) in DCM (5 mL) was added cyclopentyl carbonochloridate (52.86 mg, 0.355 mmol, 1.5 equiv) and pyridine (56.28 mg, 0.711 mmol, 3 equiv) at 0° C. The reaction was stirred at 25° C. for 2 h. Quenched with water (30 mL) and extracted with DCM (3×20 mL). The combined extracts were washed with water, brine, dried over Na₂SO₄, filtered and concentrated. The residue was purified by preparative HPLC using a XBridge Prep OBD C18 30*150 mm, 5 μm column (eluent: 26% to 42% (v/v) CH₃CN and H₂O with 10 mmol/L NH₄HCO₃) to afford the title compound cyclopentyl N-[(2R)-2-methyl-3-oxo-4-{[6-(trifluoromethyl)pyridin-2-yl]methyl}-2H-1,4-benzoxazin-7-yl]carbamate (24.7 mg, 23.14%) as a white solid. MS (ESI): mass calcd. for C₂₂H₂₂F₃N₃O₄:449.16 m/z, found: 450.10 [M+H]⁺. ¹H NMR (400 MHz, DMSO-d₆) δ 9.54 (s, 1H), 8.03-8.11 (m, 1H), 7.81 (d, J=7.7 Hz, 1H), 7.54 (d, J=8.0 Hz, 1H), 7.22 (s, 1H), 7.01 (d, J=9.4 Hz, 1H), 6.89 (d, J=8.8 Hz, 1H), 5.25 (s, 2H), 5.04-5.09 (m, 1H), 4.80-4.90 (m, 1H), 1.80-1.88 (m, 3H), 1.61-1.70 (m, 5H), 1.47 (d, J=6.7 Hz, 3H). ¹⁹F NMR (376 MHz, DMSO) δ −66.56.

### Example 305: 1-[4-benzyl-2-(fluoromethyl)-3-oxo-2H-1,4-benzoxazin-7-yl]-3-tert-butylurea

### Synthetic Scheme

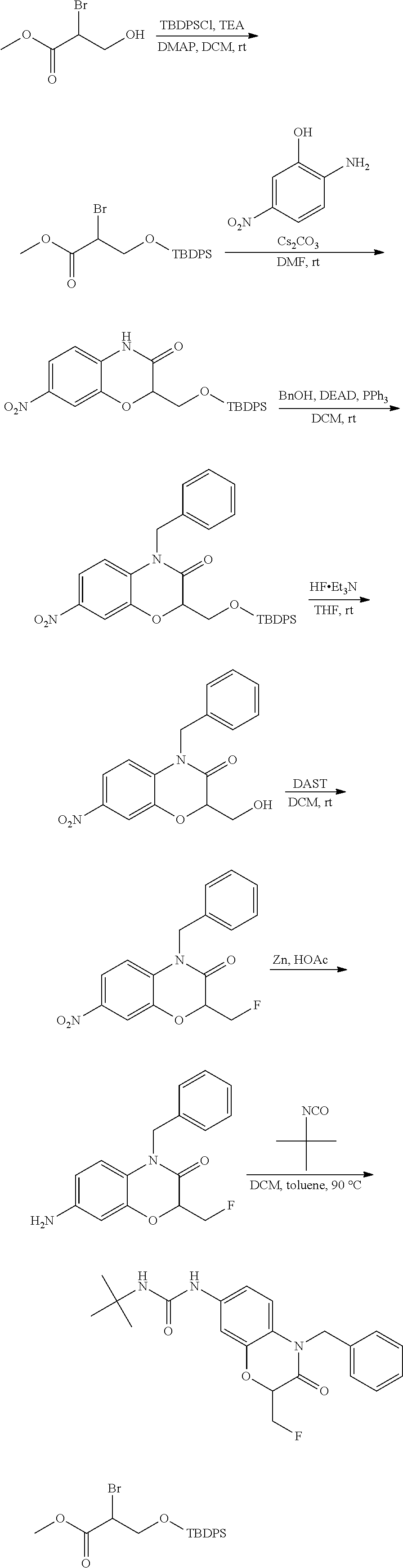

### methyl 2-bromo-3-[(tert-butyldiphenylsilyl)oxy]propanoate

To a stirred solution of methyl 2-bromo-3-hydroxypropanoate (3 g, 16.393 mmol, 1 equiv) in DCM (30 mL) was added TBDPSCl (6.76 g, 24.590 mmol, 1.5 equiv), TEA (4.98 g, 49.179 mmol, 3 equiv) and DMAP (0.20 g, 1.639 mmol, 0.1 equiv) in portions at room temperature under air atmosphere. The resulting mixture was stirred for 2 h at room temperature under air atmosphere. The reaction was monitored by LCMS. Quenched with water (150 mL) and extracted with DCM (3×150 mL). The combined extracts were washed with water, brine, dried over Na₂SO₄, filtered and concentrated. The residue was purified by silica gel column chromatography, eluted with PE/EA (0-100%) to afford methyl 2-bromo-3-[(tert-butyldiphenylsilyl)oxy]propanoate (6.5 g, 94.1%) as a yellow oil. LC/MS (ESI): mass calcd. for C₂₀H₂₅BrO₃Si: 420.1 m/z, found: 421.1, 423.1 [M+H, M+H+2]*.

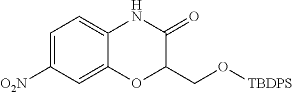

### 2-{[(tert-butyldiphenylsilyl)oxy]methyl}-7-nitro-2,4-dihydro-1,4-benzoxazin-3-one

To a stirred solution of methyl 2-bromo-3-[(tert-butyldiphenylsilyl)oxy]propanoate (8 g, 18.984 mmol, 1 equiv) in DMF (80 mL) was added 2-amino-5-nitrophenol (2.93 g, 18.984 mmol, 1 equiv) and K₂CO₃ (5.25 g, 37.968 mmol, 2 equiv) in portions at room temperature under air atmosphere. The resulting mixture was stirred for 2 h at 80° C. under air atmosphere. The reaction was monitored by LCMS. After cooling down to rt, the reaction was quenched with water (400 mL) and extracted with EA (3×300 mL). The combined extracts were washed with water, brine, dried over Na₂SO₄, filtered and concentrated. The residue was purified by silica gel column chromatography, eluted with PE/EA (0-100%) to afford 2-{[(tert-butyldiphenylsilyl)oxy]methyl}-7-nitro-2,4-dihydro-1,4-benzoxazin-3-one (2.8 g, 31.88%) as a yellow solid. LC/MS (ESI): mass calcd. for C₂₅H₂₆N₂O₅Si: 462.2 m/z, found: 461.2 [M+H]⁺.

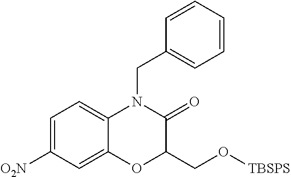

### 4-benzyl-2-{[(tert-butyldiphenylsilyl)oxy]methyl}-7-nitro-2H-1,4-benzoxazin-3-one

To a stirred solution of 2-{[(tert-butyldiphenylsilyl)oxy]methyl}-7-nitro-2,4-dihydro-1,4-benzoxazin-3-one (1.2 g, 2.594 mmol, 1 equiv) and benzyl alcohol (0.28 g, 2.594 mmol, 1 equiv) in DCM was added PPh₃ (0.68 g, 2.594 mmol, 1 equiv) in portions at room temperature under nitrogen atmosphere. The resulting mixture was stirred for 30 min at room temperature under nitrogen atmosphere. To the above mixture was added DEAD (0.68 g, 3.891 mmol, 1.5 equiv) in portions at 0° C. The resulting mixture was stirred for additional 2 h at room temperature. The reaction was monitored by LCMS. Quenched with water (50 mL) and extracted with DCM (3×50 mL). The combined extracts were washed with water, brine, dried over Na₂SO₄, filtered and concentrated. The residue was purified by silica gel column chromatography, eluted with PE/EA (0-100%) to afford 4-benzyl-2-{[(tert-butyldiphenylsilyl)oxy]methyl}-7-nitro-2H-1,4-benzoxazin-3-one (800 mg, 55.80%) as a yellow oil. LC/MS (ESI): mass calcd. for C₃₂H₃₂N₂O₅Si: 552.2 m/z, found: 551.3 [M+H]⁺.

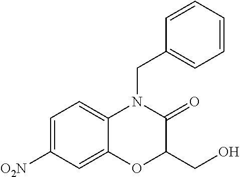 [SMILES: O=C1C(CO)Oc2cc([N+](=O)[O-])ccc2N1Cc1ccccc1]

### 4-benzyl-2-(hydroxymethyl)-7-nitro-2H-1,4-benzoxazin-3-one

To a stirred solution of 4-benzyl-2-{[(tert-butyldiphenylsilyl)oxy]methyl}-7-nitro-2H-1,4-benzoxazin-3-one (800 mg, 1.447 mmol, 1 equiv) in THF (8 mL) was added Et₃N·3HF (350.02 mg, 2.171 mmol, 1.5 equiv) in portions at room temperature under air atmosphere. The resulting mixture was stirred for 3 h at room temperature under air atmosphere. The reaction was monitored by LCMS. Quenched with water (40 mL) and extracted with EA (3×40 mL). The combined extracts were washed with water, brine, dried over Na₂SO₄, filtered and concentrated. The residue was purified by silica gel column chromatography, eluted with PE/EA (0-100%) to afford 4-benzyl-2-(hydroxymethyl)-7-nitro-2H-1,4-benzoxazin-3-one (320 mg, 70.34%) as a yellow oil. LC/MS (ESI): mass calcd. for C₁₆H₁₄N₂O₅:314.1 m/z, found: 315.2 [M+H]⁺.

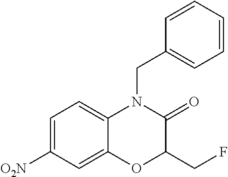 [SMILES: O=C1C(CF)Oc2cc([N+](=O)[O-])ccc2N1Cc1ccccc1]

### 4-benzyl-2-(fluoromethyl)-7-nitro-2H-1,4-benzoxazin-3-one

To a stirred solution of 4-benzyl-2-(hydroxymethyl)-7-nitro-2H-1,4-benzoxazin-3-one (320 mg, 1.018 mmol, 1 equiv) in DCM (3 mL) was added DAST (328.23 mg, 2.036 mmol, 2 equiv) dropwise at 0° C. under air atmosphere. The resulting mixture was stirred for 3 h at 0° C. under air atmosphere. The reaction was monitored by LCMS. Quenched with water (30 mL) and extracted with EA (3×30 mL). The combined extracts were washed with water, brine, dried over Na₂SO₄, filtered and concentrated. The residue was purified by silica gel column chromatography, eluted with PE/EA (0-100%) to afford 4-benzyl-2-(fluoromethyl)-7-nitro-2H-1,4-benzoxazin-3-one (130 mg, 40.37%) as a yellow oil. LC/MS (ESI): mass calcd. for C₁₆H₁₃FN₂O₄:316.1 m/z, found: 317.20 [M+H]⁺.

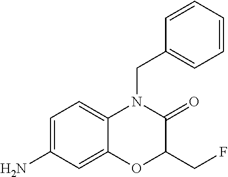 [SMILES: Nc1ccc2c(c1)OC(CF)C(=O)N2Cc1ccccc1]

### 7-amino-4-benzyl-2-(fluoromethyl)-2H-1,4-benzoxazin-3-one

To a stirred solution of 4-benzyl-2-(fluoromethyl)-7-nitro-2H-1,4-benzoxazin-3-one (130 mg, 0.411 mmol, 1 equiv) in AcOH (1.5 mL) was added Zn (268.72 mg, 4.110 mmol, 10 equiv) in portions at room temperature under air atmosphere. The resulting mixture was stirred for 3 h at room temperature under air atmosphere. The reaction was monitored by LCMS. Quenched with water (10 mL) and extracted with EA (3×10 mL). The combined extracts were washed with water, brine, dried over Na₂SO₄, filtered and concentrated. This resulted in 7-amino-4-benzyl-2-(fluoromethyl)-2H-1,4-benzoxazin-3-one (130 mg, 110.47%) as a yellow solid. LC/MS (ESI): mass calcd. for C₁₆H₁₅FN₂O₂:286.1 m/z, found: 287.20 [M+H]⁺.

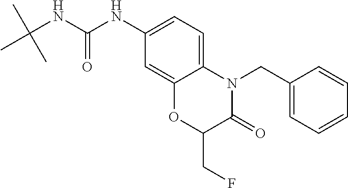 [SMILES: CC(C)(C)NC(=O)Nc1ccc2c(c1)OC(CF)C(=O)N2Cc1ccccc1]

### 1-[4-benzyl-2-(fluoromethyl)-3-oxo-2H-1,4-benzoxazin-7-yl]-3-tert-butylurea

To a stirred solution of 7-amino-4-benzyl-2-(fluoromethyl)-2H-1,4-benzoxazin-3-one (120 mg, 0.419 mmol, 1 equiv) in DCM/Toluene (2:1, 1.5 mL) was added 2-isocyanato-2-methylpropane (124.65 mg, 1.257 mmol, 3 equiv) dropwise at room temperature under air atmosphere. The resulting mixture was stirred overnight at 90° C. under air atmosphere. The reaction was monitored by LCMS. The mixture was allowed to cool down to room temperature. Quenched with water (10 mL) and extracted with DCM (3×10 mL). The combined extracts were washed with water, brine, dried over Na₂SO₄, filtered and concentrated. The crude product was purified by Prep-HPLC using YMC-Actus Tiart ExRS C18 150 mm×30 mm×5 μm column (eluent: 23% to 40% (v/v) CH₃CN and H₂O with 10 mmol/L NH₄HCO₃) to afford 1-[4-benzyl-2-(fluoromethyl)-3-oxo-2H-1,4-benzoxazin-7-yl]-3-tert-butylurea (13.7 mg, 8.25%) as a white solid. LC/MS (ESI): mass calcd. for C₂₁H₂₄FN₃O₃:385.2 m/z, found: 386.10 [M+H]⁺. ¹H NMR (400 MHz, DMSO-d₆) δ 8.24 (s, 1H), 7.35-7.21 (m, 6H), 6.83 (d, J=8.8 Hz, 1H), 6.74 (d, J=8.8 Hz, 1H), 5.97 (s, 1H), 5.21-4.81 (m, 5H), 1.26 (s, 9H). ¹⁹F NMR (376 MHz, DMSO-d₆) δ −231.52.

### Example 306 and Example 307: 1-(tert-butyl)-3-((R)-2-methyl-3-oxo-4-((S)-1-(6-(trifluoromethyl)pyrazin-2-yl)ethyl)-3,4-dihydro-2H-benzo[b][1,4]oxazin-7-yl)urea and 1-(tert-butyl)-3-((R)-2-methyl-3-oxo-4-((R)-1-(6-(trifluoromethyl)pyrazin-2-yl)ethyl)-3,4-dihydro-2H-benzo[b][1,4]oxazin-7-yl)urea

### Synthetic Scheme

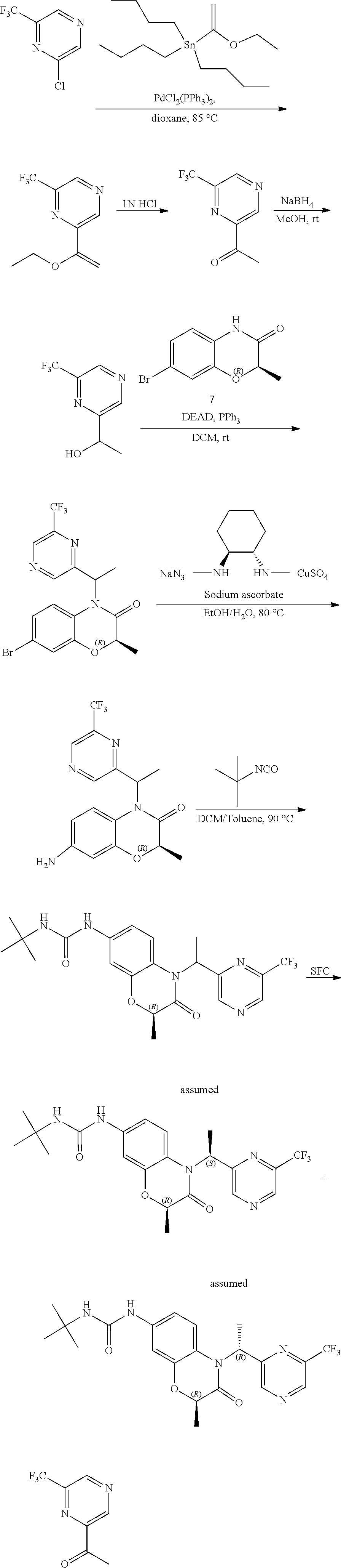

### 1-(6-(trifluoromethyl)pyrazin-2-yl)ethan-1-one

To a solution of 2-chloro-6-(trifluoromethyl)pyrazine (1 g, 5.479 mmol, 1 equiv) and bis(triphenylphosphine) palladium chloride (0.38 g, 0.548 mmol, 0.1 equiv) in dioxane (10 mL) was added tributyl(1-ethoxyethenyl)stannane (2.97 g, 8.219 mmol, 1.5 equiv) under N₂. The reaction was stirred at 85° C. under N₂ for 3 h. The mixture was allowed to cool down to rt, and to the above mixture was added 1 N HCl (5 mL, 16.456 mmol). The resulting mixture was stirred for additional 1 h at rt. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give 1-[6-(trifluoromethyl)pyrazin-2-yl]ethanone (640 mg, 60.21%) as a yellow oil. MS (ESI): mass calcd. for C₇H₅F₃N₂O: 190.04 m/z, found 191.00[M+H]⁺.

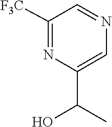

### 1-(6-(trifluoromethyl)pyrazin-2-yl)ethan-1-ol

To a solution of 1-[6-(trifluoromethyl)pyrazin-2-yl]ethanone (600 mg, 3.156 mmol, 1 equiv) in MeOH (10 mL) was added NaBH₄ (358.15 mg, 9.468 mmol, 3 equiv) at 0° C. The reaction was stirred at rt for 1 h. Quenched with water (50 mL) and extracted with DCM (3×50 mL). The combined extracts were washed with water, brine, dried over Na₂SO₄, filtered and concentrated. The residue was purified by column chromatography on silica gel with EA/PE (0-100%) to give 1-[6-(trifluoromethyl)pyrazin-2-yl]ethanol (210 mg, 34.63%) as a yellow oil. MS (ESI): mass calcd. for C₇H₇F₃N₂O: 192.05 m/z, found 193.15 [M+H]⁺.

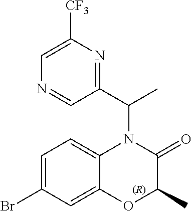

### (2R)-7-bromo-2-methyl-4-(1-(6-(trifluoromethyl)pyrazin-2-yl)ethyl)-2H-benzo[b][1,4]oxazin-3(4H)-one

To a solution of 1-[6-(trifluoromethyl)pyrazin-2-yl]ethanol (200 mg, 1.041 mmol, 1 equiv) in DCM (10 mL) was added (2R)-7-bromo-2-methyl-2,4-dihydro-1,4-benzoxazin-3-one (251.97 mg, 1.041 mmol, 1 equiv) and PPh₃ (409.53 mg, 1.561 mmol, 1.5 equiv). The reaction was stirred at rt under N₂ for 0.5 h. To the above mixture was added DEAD (271.92 mg, 1.561 mmol, 1.5 equiv) at 0° C. The resulting mixture was stirred for additional 1 h at rt. Quenched with water (30 mL) and extracted with DCM (3×30 mL). The combined extracts were washed with water, brine, dried over Na₂SO₄, filtered and concentrated. The residue was purified by column chromatography on silica gel with EA/PE (0-100%) to give (2R)-7-bromo-2-methyl-4-{1-[6-(trifluoromethyl)pyrazin-2-yl]ethyl}-2H-1,4-benzoxazin-3-one (162 mg, 37.39%) as a yellow oil. MS (ESI): mass calcd. for C₁₆H₁₃BrF₃N₃O₂: 415.01 m/z, found: 415.90, 417.90 [M+H, M+H+2]*.

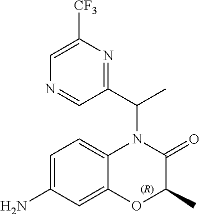

### (2R)-7-amino-2-methyl-4-(1-(6-(trifluoromethyl)pyrazin-2-yl)ethyl)-2H-benzo[b][1,4]oxazin-3(4H)-one

To a solution of (2R)-7-bromo-2-methyl-4-{1-[6-(trifluoromethyl)pyrazin-2-yl]ethyl}-2H-1,4-benzoxazin-3-one (150 mg, 0.360 mmol, 1 equiv) in EtOH/H₂O (4:1, 10 mL) was added (1R,2R)—N1,N2-dimethylcyclohexane-1,2-diamine (30.76 mg, 0.216 mmol, 0.6 equiv), azidosodium (70.29 mg, 1.080 mmol, 3 equiv), CuSO₄(57.52 mg, 0.360 mmol, 1 equiv) and sodium ascorbate (143.52 mg, 0.720 mmol, 2 equiv). The reaction was stirred at 80° C. under N₂ for 3 h. After cooling down to rt, the reaction was quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-7-amino-2-methyl-4-{1-[6-(trifluoromethyl)pyrazin-2-yl]ethyl}-2H-1,4-benzoxazin-3-one (100 mg, 78.75%) as a yellow oil. MS (ESI): mass calcd. for C₁₆H₁₅F₃N₄O₂: 352.11 m/z, found 353.10 [M+H]⁺.

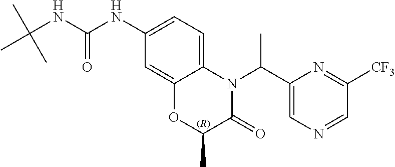

### 1-(tert-butyl)-3-((2R)-2-methyl-3-oxo-4-(1-(6-(trifluoromethyl)pyrazin-2-yl)ethyl)-3,4-dihydro-2H-benzo[b][1,4]oxazin-7-yl)urea

To a solution of (2R)-7-amino-2-methyl-4-{1-[6-(trifluoromethyl)pyrazin-2-yl]ethyl}-2H-1,4-benzoxazin-3-one (100 mg, 0.284 mmol, 1 equiv) in DCM/Toluene (2:1, 15 mL) was added 2-isocyanato-2-methylpropane (281.37 mg, 2.840 mmol, 10 equiv). The reaction was stirred at 90° C. under N₂ for 3 h. After cooling down to rt, the reaction was quenched with water (100 mL) and extracted with DCM (3×100 mL). The combined extracts were washed with water, brine, dried over Na₂SO₄, filtered and concentrated. The residue was purified by preparative HPLC using a XBridge Prep OBD C18 Column, 30*150 mm, 5 μm column (eluent: 41% to 63% (v/v) CH₃CN and H₂O with 10 mmol/L NH₄HCO₃) to afford the title compound 3-tert-butyl-1-[(2R)-2-methyl-3-oxo-4-{1-[6-(trifluoromethyl)pyrazin-2-yl]ethyl}-2H-1,4-benzoxazin-7-yl]urea (40 mg, 31.21%) as a white solid. MS (ESI): mass calcd. for C₂₁H₂₄F₃N₅O₃: 451.18 m/z, found 452.00 [M+H]⁺.

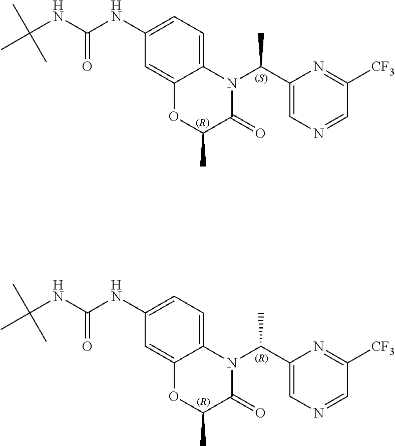

### 1-(tert-butyl)-3-((R)-2-methyl-3-oxo-4-((S)-1-(6-(trifluoromethyl)pyrazin-2-yl)ethyl)-3,4-dihydro-2H-benzo[b][1,4]oxazin-7-yl)urea and 1-(tert-butyl)-3-((R)-2-methyl-3-oxo-4-((R)-1-(6-(trifluoromethyl)pyrazin-2-yl)ethyl)-3,4-dihydro-2H-benzo[b][1,4]oxazin-7-yl)urea

A sample of 3-tert-butyl-1-[(2R)-2-methyl-3-oxo-4-{1-[6-(trifluoromethyl)pyrazin-2-yl]ethyl}-2H-1,4-benzoxazin-7-yl]urea (40 mg) was separated by chiral-HPLC using a CHIRALPAK IE, 2*25 cm, 5 μm column (eluent: 90% to 90% (v/v) HEX:DCM=3:1(0.3% IPAmine) and IPA) to yield first example 3-tert-butyl-1-[(2R)-2-methyl-3-oxo-4-[(1S)-1-[6-(trifluoromethyl)pyrazin-2-yl]ethyl]-2H-1,4-benzoxazin-7-yl]urea (16.3 mg, 40.75%) as a white solid. MS(ESI): mass calcd. for C₂₁H₂₄F₃N₅O₃: 451.18 m/z, found: 452.05 [M+H]⁺. ¹H NMR (400 MHz, DMSO-d₆) δ 9.10 (s, 1H), 9.00 (s, 1H), 8.25 (s, 1H), 7.25 (d, J=2.3 Hz, 1H), 6.77-6.90 (m, 2H), 6.00-6.05 (m, 1H), 5.93-5.99 (m, 1H), 4.67-4.70 (m, 1H), 1.75-1.85 (m, 3H), 1.36-1.41 (m, 3H), 1.28 (s, 9H). ¹⁹F NMR (376 MHz, DMSO) δ(ppm): −66.4059. And to yield second example 3-tert-butyl-1-[(2R)-2-methyl-3-oxo-4-[(1R)-1-[6-(trifluoromethyl)pyrazin-2-yl]ethyl]-2H-1,4-benzoxazin-7-yl]urea (15 mg, 37.50%) as a white solid. MS(ESI): mass calcd. for C₂₁H₂₄F₃N₅O₃: 451.18 m/z, found: 452.05 [M+H]⁺. ¹H NMR (400 MHz, DMSO-d₆) δ 9.08 (s, 1H), 8.98 (s, 1H), 8.25 (s, 1H), 7.24 (d, J=2.4 Hz, 1H), 6.89-7.00 (m, 1H), 6.74-6.89 (m, 1H), 5.87-6.10 (m, 2H), 4.60-4.75 (m, 1H), 1.80-1.87 (m, 3H), 1.36-1.44 (m, 3H), 1.28 (s, 9H). ¹⁹F NMR (376 MHz, DMSO) δ(ppm): −66.3907.

### Example 308: N-(1-benzyl-2-oxo-1,2-dihydroquinoxalin-6-yl)-2,2-dimethylcyclopentane-1-carboxamide

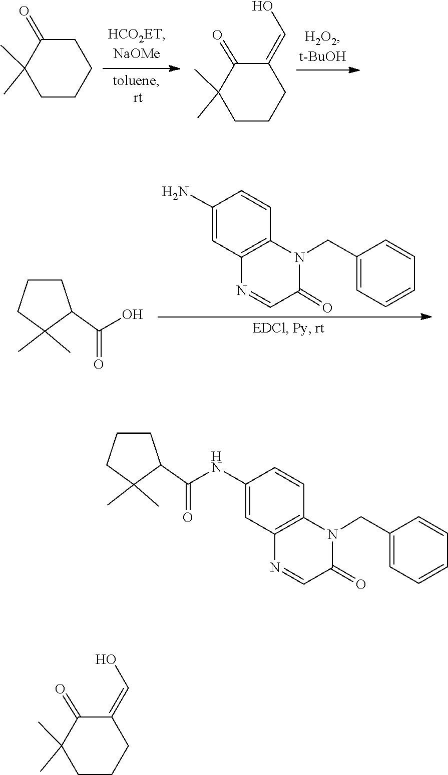

### (Z)-6-(hydroxymethylene)-2,2-dimethylcyclohexan-1-one

A solution of sodium methoxide (406 mg, 7.528 mmol, 1.9 equiv) in toluene (8 mL) was treated with 2,2-dimethylcyclohexan-1-one (500 mg, 3.962 mmol, 1 equiv). The solution was stirred at rt for 30 min. Ethyl formate (733 mg, 9.905 mmol, 2.5 equiv) was added into the reaction. The solution was stirred at rt for 12 h. The mixture was acidified to pH 7 with 2N HCl. The reaction mixture was quenched by ice water (20 mL) and extracted with DCM (3×20 mL). The organic layers were combined, dried over anhydrous sodium sulfate, filtered and concentrated under vacuum to yield a crude product which was directly purified by flash chromatography (0-60% EA/PE) to afford (Z)-6-(hydroxymethylene)-2,2-dimethylcyclohexan-1-one (300 mg, 49.10%) as white solid. MS (ESI): mass calcd. for C₉H₁₄O₂, 154.10 m/z, found 153.00 [M−H]⁻.

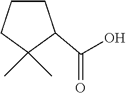

### 2,2-dimethylcyclopentane-1-carboxylic acid

A solution of (Z)-6-(hydroxymethylene)-2,2-dimethylcyclohexan-1-one (300 mg, 0.065 mmol, 1 equiv) in 2-methyl-2-propanol (6 mL) was treated with hydrogen peroxide (0.6 mL, 0.235 mmol, 3.63 equiv). The solution was stirred at rt for overnight. The mixture was acidified to pH 5 with NaHCO₃(aq.). The reaction mixture was quenched by ice water (20 mL) and extracted with Et₂₀ (3×20 mL). The organic layers were combined, dried over anhydrous sodium sulfate, filtered and concentrated under vacuum to yield a crude product 2,2-dimethylcyclopentane-1-carboxylic acid (70 mg, 25.36%) as white oil. MS (ESI): mass calcd. for C₈H₁₄O₂, 142.10 m/z, found 141.00 [M−H]⁻.

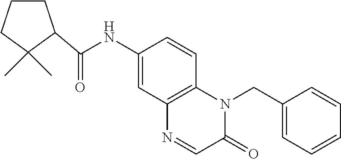 [SMILES: CC1(C)CCCC1C(=O)Nc1ccc2c(c1)ncc(=O)n2Cc1ccccc1]

### N-(1-benzyl-2-oxo-1,2-dihydroquinoxalin-6-yl)-2,2-dimethylcyclopentane-1-carboxamide

To a mixture of 2,2-dimethylcyclopentane-1-carboxylic acid (70 mg, 0.492 mmol, 1 equiv) and 6-amino-1-benzylquinoxalin-2-one (123 mg, 0.492 mmol, 1 equiv) in pyridine (5 mL) was stirred, and added EDCI (189 mg, 0.984 mmol, 2 equiv). The reaction was stirred at rt for 4 h. The resulting mixture was concentrated under vacuum. The crude product was purified by reverse phase flash with the following conditions to afford N-(1-benzyl-2-oxo-1,2-dihydroquinoxalin-6-yl)-2,2-dimethylcyclopentane-1-carboxamide (21.4 mg, 11.55%) as yellow solid. MS (ESI): mass calcd. for C₂₃H₂₅N₃O₂, 375.19 m/z, found 376.10 [M+H]⁺. ¹H NMR (400 MHz, DMSO-d₆) δ 9.93 (s, 1H), 8.35 (s, 1H), 8.22 (d, J=2.4 Hz, 1H), 7.71 (dd, J=9.1, 2.5 Hz, 1H), 7.43 (d, J=9.0 Hz, 1H), 7.29-7.37 (m, 2H), 7.25-7.27 (m, 3H), 5.47 (s, 2H), 2.46 (t, J=7.7 Hz, 1H), 1.96-2.03 (m, 1H), 1.80-1.89 (m, 1H), 1.70-1.75 (m, 1H), 1.60-1.64 (m, 1H), 1.51-1.56 (m, 1H), 1.36-1.45 (m, 1H), 1.10 (s, 3H), 0.91 (s, 3H).

### Example 309: 1-(tert-butyl)-3-(1-(2,3-dihydro-1H-inden-1-yl)-2-oxo-1,2,3,4-tetrahydroquinolin-6-yl)urea

### Synthetic Scheme

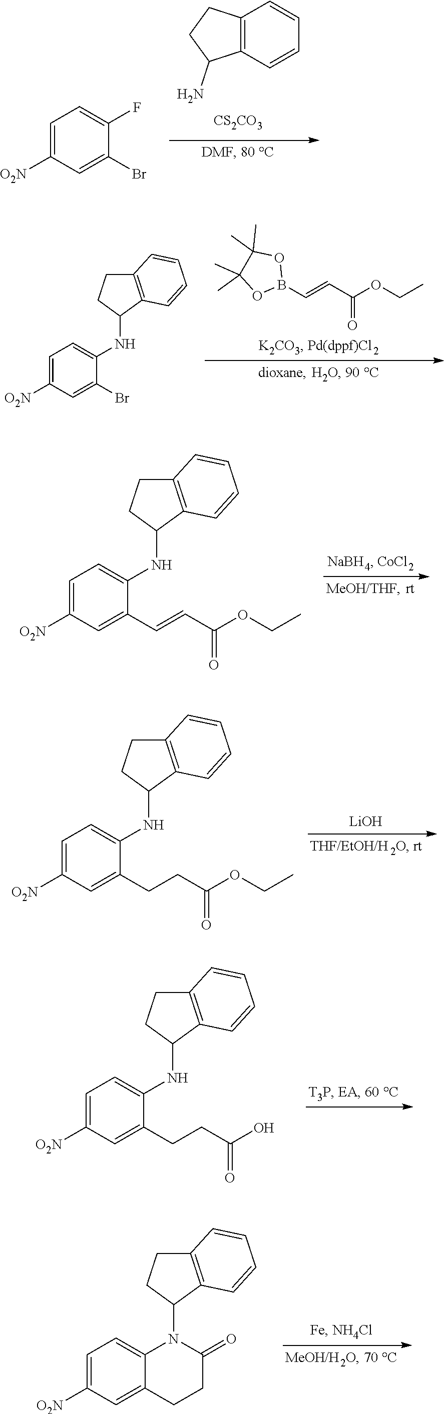

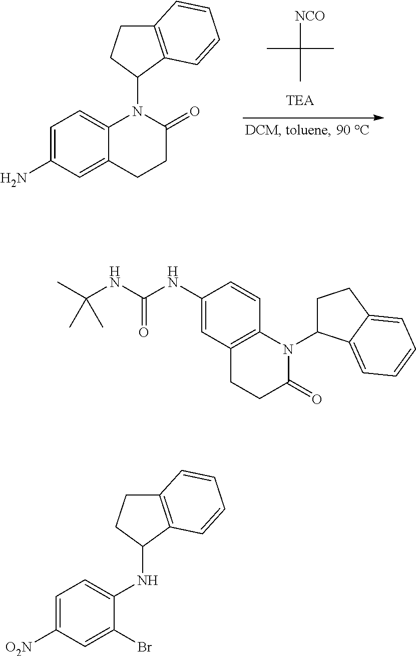

### N-(2-bromo-4-nitrophenyl)-2,3-dihydro-1H-inden-1-amine

To a stirred mixture of 2-bromo-1-fluoro-4-nitrobenzene (4.3 g, 19.546 mmol, 1 equiv) and 2,3-dihydro-1H-inden-1-amine (2.60 g, 19.546 mmol, 1 equiv) in DMF (50 mL) was added Cs₂CO₃ (15.92 g, 48.865 mmol, 2.5 equiv) in portions at 25° C. under air atmosphere. The resulting mixture was stirred for 2 h at 80° C. Desired product could be detected by LCMS. The mixture was diluted with water (200 mL). The resulting mixture was extracted with EA (3×200 mL). The combined organic layers were dried over anhydrous Na₂SO₄. After filtration, the filtrate was concentrated under reduced pressure. The residue was purified by silica gel column chromatography, eluted with PE/EA (0-100%) to afford N-(2-bromo-4-nitrophenyl)-2,3-dihydro-1H-inden-1-amine (3.6 g, 55.28%) as a white solid. LC/MS (ESI): mass calcd. for C₁₅H₁₃BrN₂O₂: 332.02 m/z, found 334.90 [M+H, M+H+2]⁺.

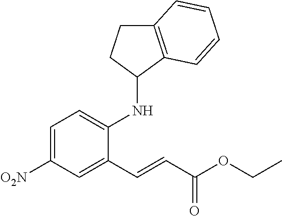 [SMILES: CCOC(=O)/C=C/c1cc([N+](=O)[O-])ccc1NC1CCc2ccccc21]

### ethyl (E)-3-(2-((2,3-dihydro-1H-inden-1-yl)amino)-5-nitrophenyl)acrylate

To a stirred mixture of N-(2-bromo-4-nitrophenyl)-2,3-dihydro-1H-inden-1-amine (1.5 g, 4.502 mmol, 1 equiv) and ethyl (2E)-3-(4,4,5,5-tetramethyl-1,3,2-dioxaborolan-2-yl)prop-2-enoate (1.53 g, 6.753 mmol, 1.5 equiv) in dioxane (15 mL) and H₂O (1.5 mL) was added K₂CO₃ (1.56 g, 11.255 mmol, 2.5 equiv) and Pd(dppf)Cl₂ (329.42 mg, 0.450 mmol, 0.1 equiv) in portions at 25° C. under nitrogen atmosphere. The resulting mixture was stirred for 2 h at 90° C. under N₂. Desired product could be detected by LCMS. After cooling down to rt, the mixture was diluted with water (100 mL). The resulting mixture was extracted with EA (3×100 mL). The combined organic layers were dried over anhydrous Na₂SO₄. After filtration, the filtrate was concentrated under reduced pressure. The residue was purified by silica gel column chromatography, eluted with PE/EA (0-100%) to afford ethyl (E)-3-(2-((2,3-dihydro-1H-inden-1-yl)amino)-5-nitrophenyl)acrylate (630 mg, 39.71%) as a white solid. LC/MS (ESI): mass calcd. for C₂₀H₂₀N₂O₄: 352.14 m/z, found 353.15 [M+H]⁺.

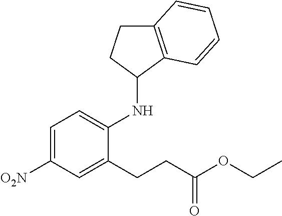 [SMILES: CCOC(=O)CCc1cc([N+](=O)[O-])ccc1NC1CCc2ccccc21]

### ethyl 3-(2-((2,3-dihydro-1H-inden-1-yl)amino)-5-nitrophenyl)propanoate

To a stirred mixture of ethyl (E)-3-(2-((2,3-dihydro-1H-inden-1-yl)amino)-5-nitrophenyl)acrylate (610 mg, 1.731 mmol, 1 equiv) and CoCl₂ (112.37 mg, 0.866 mmol, 0.5 equiv) in MeOH (0.5 mL) and THF (10 mL) was added NaBH₄ (130.97 mg, 3.462 mmol, 2 equiv) in portions at 0° C. under N₂ atmosphere. The resulting mixture was stirred for 2 h at room temperature. Desired product could be detected by LCMS. The mixture was diluted with water (50 mL). The resulting mixture was extracted with EA (3×50 mL). The combined organic layers were dried over anhydrous Na₂SO₄. After filtration, the filtrate was concentrated under reduced pressure. The residue was purified by silica gel column chromatography, eluted with PE/EA (0-100%) to afford ethyl 3-(2-((2,3-dihydro-1H-inden-1-yl)amino)-5-nitrophenyl)propanoate (200 mg, 35.89%) as a white solid. LC/MS (ESI): mass calcd. for C₁₈H₁₈N₂O₄: 354.16 m/z, found 355.15 [M+H]⁺.

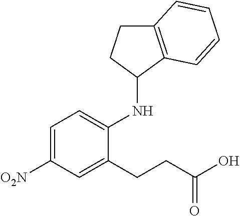 [SMILES: O=C(O)CCc1cc([N+](=O)[O-])ccc1NC1CCc2ccccc21]

### 3-(2-((2,3-dihydro-1H-inden-1-yl)amino)-5-nitrophenyl)propanoic acid

To a stirred mixture of ethyl 3-(2-((2,3-dihydro-1H-inden-1-yl)amino)-5-nitrophenyl)propanoate (210 mg, 0.593 mmol, 1 equiv) in THF (2 mL), EtOH (2 mL) and H₂O (2 mL) was added LiOH (42.57 mg, 1.779 mmol, 3 equiv) at 0° C. under air atmosphere. The resulting mixture was stirred for 2 h at room temperature. Desired product could be detected by LCMS. The mixture was basified to PH=5 with 2N HCl and diluted with water (20 mL). The resulting mixture was extracted with EA (3×20 mL). The combined organic layers were dried over anhydrous Na₂SO₄. After filtration, the filtrate was concentrated under reduced pressure to afford 3-(2-((2,3-dihydro-1H-inden-1-yl)amino)-5-nitrophenyl)propanoic acid (180 mg, 93.08%) as a white solid. LC/MS (ESI): mass calcd. for C₂₅H₃₄N₄O₂: 326.13 m/z, found 327.15 [M+H]+.

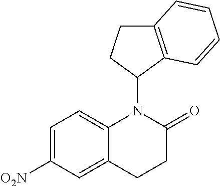 [SMILES: O=C1CCc2cc([N+](=O)[O-])ccc2N1C1CCc2ccccc21]

### 1-(2,3-dihydro-1H-inden-1-yl)-6-nitro-3,4-dihydroquinolin-2(1H)-one

To a stirred mixture of 3-(2-((2,3-dihydro-1H-inden-1-yl)amino)-5-nitrophenyl)propanoic acid (170 mg, 0.521 mmol, 1 equiv) in T₃P (1 mL) at 0° C. under air atmosphere. The resulting mixture was stirred for 2 h at room temperature. Desired product could be detected by LCMS. The mixture was diluted with water (10 mL). The resulting mixture was extracted with EA (3×10 mL). The organic layers were combined, dried over anhydrous sodium sulfate, filtered and concentrated. The residue obtained was purified by silica gel chromatography (0-100% ethyl acetate/petroleum ether) to afford the 1-(2,3-dihydro-1H-inden-1-yl)-6-nitro-3,4-dihydroquinolin-2(1H)-one (150 mg, 93.39%) as a white solid. LC/MS (ESI): mass calcd. for C₁₈H₁₆N₂O₃: 308.12 m/z, found 309.05[M+H]⁺.

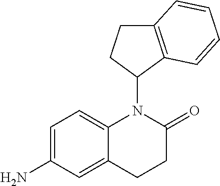 [SMILES: Nc1ccc2c(c1)CCC(=O)N2C1CCc2ccccc21]

### 6-amino-1-(2,3-dihydro-1H-inden-1-yl)-3,4-dihydroquinolin-2(1H)-one

To a stirred mixture of 1-(2,3-dihydro-1H-inden-1-yl)-6-nitro-3,4-dihydroquinolin-2(1H)-one (170 mg, 0.551 mmol, 1 equiv) in MeOH (2 mL) and H₂O (0.2 mL) was added Fe (307.90 mg, 5.510 mmol, 10 equiv) and NH₄C₁ (294.91 mg, 5.510 mmol, 10 equiv) in portions at room temperature under air atmosphere. The resulting mixture was stirred for 2 h at 70° C. Desired product could be detected by LCMS. After cooling down to rt, the mixture was diluted with water (10 mL). The resulting mixture was extracted with EA (3×10 mL). The organic layers were combined, dried over anhydrous sodium sulfate, filtered and concentrated. The residue obtained was purified by silica gel chromatography (0-20% ethyl acetate/petroleum ether) to afford the 6-amino-1-(2,3-dihydro-1H-inden-1-yl)-3,4-dihydroquinolin-2(1H)-one (120 mg, 78.19%) as a yellow solid. LC/MS (ESI): mass calcd. for C₁₈H₁₈N₂O: 278.14 m/z, found 279.15[M+H]⁺.

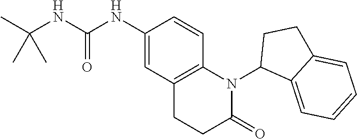 [SMILES: CC(C)(C)NC(=O)Nc1ccc2c(c1)CCC(=O)N2C1CCc2ccccc21]

### 1-(tert-butyl)-3-(1-(2,3-dihydro-1H-inden-1-yl)-2-oxo-1,2,3,4-tetrahydroquinolin-6-yl)urea

To a stirred mixture of 6-amino-1-(2,3-dihydro-1H-inden-1-yl)-3,4-dihydroquinolin-2(1H)-one (110 mg, 0.395 mmol, 1 equiv) in DCM/Toluene (2:1, 2 mL) was added 2-isocyanato-2-methylpropane (117.53 mg, 1.185 mmol, 3 equiv) dropwise at room temperature under nitrogen atmosphere. The resulting mixture was stirred for 2 h at 90° C. Desired product could be detected by LCMS. After cooling down to rt, the mixture was diluted with water (10 mL). The resulting mixture was extracted with DCM (3×10 mL). The organic layers were combined, dried over anhydrous sodium sulfate, filtered and concentrated. The residue was purified by preparative HPLC using a X Bridge Prep OBD C18 150 mm×30 mm×5 μm column (eluent: 35% to 65% (v/v) CH₃CN and H₂O with 10 mmol/L NH₄HCO₃) to afford the 1-(tert-butyl)-3-(1-(2,3-dihydro-1H-inden-1-yl)-2-oxo-1,2,3,4-tetrahydroquinolin-6-yl)urea (31.6 mg, 21.16%) as a white solid. LC/MS (ESI): mass calcd. for C₂₃H₂₇N₃O₂: 377.21 m/z, found 378.15[M+H]⁺. ¹H NMR (300 MHz, DMSO-d₆) δ 8.06 (s, 1H), 7.32-7.29 (m, 2H), 7.20 (t, J=7.3 Hz, 1H), 7.10 (t, J=7.3 Hz, 1H), 6.94 (d, J=7.5 Hz, 1H), 6.82 (d, J=7.5 Hz, 1H), 6.36 (s, 2H), 5.92 (m, 1H), 3.15-3.11 (m, 1H), 2.98-2.94 (m, 1H), 2.85-2.80 (m, 2H), 2.57-2.55 (m, 2H), 2.43-2.27 (m, 2H), 1.25 (s, 9H).

### Example 310 and Example 311: 1-(tert-butyl)-3-((R)-2-methyl-3-oxo-4-((S)-1-(2-(trifluoromethyl)pyrimidin-4-yl)ethyl)-3,4-dihydro-2H-benzo[b][1,4]oxazin-7-yl)urea and 1-(tert-butyl)-3-((R)-2-methyl-3-oxo-4-((R)-1-(2-(trifluoromethyl)pyrimidin-4-yl)ethyl)-3,4-dihydro-2H-benzo[b][1,4]oxazin-7-yl)urea

### Synthetic Scheme

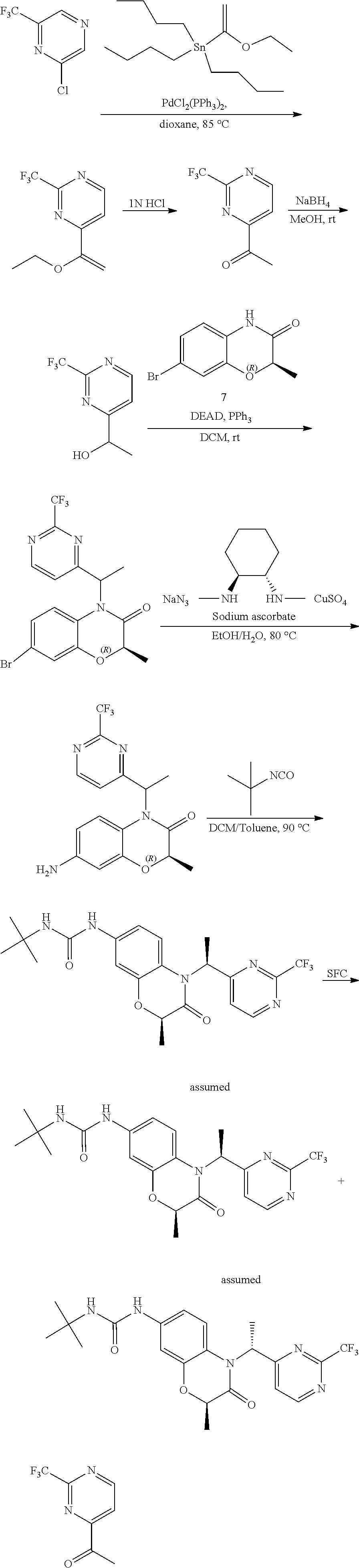

### 1-(2-(trifluoromethyl)pyrimidin-4-yl)ethan-1-one

To a solution of 4-chloro-2-(trifluoromethyl)pyrimidine (1 g, 5.479 mmol, 1 equiv) and bis(triphenylphosphine) palladium chloride (0.38 g, 0.548 mmol, 0.1 equiv) in dioxane (10 mL) was added tributyl(1-ethoxyethenyl)stannane (2.97 g, 8.219 mmol, 1.5 equiv) under N₂. The reaction was stirred at 85° C. under N₂ for 3 h. The mixture was allowed to cool down to rt., and to the above mixture was added 1 N HCl (5 mL). The resulting mixture was stirred for additional 1 h at rt. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give 1-[2-(trifluoromethyl)pyrimidin-4-yl]ethanone (500 mg, 47.04%) as a yellow oil. MS (ESI): mass calcd. for C₇H₅F₃N₂O: 190.04 m/z, found 191.45 [M+H]⁺

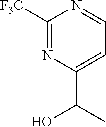

### 1-(2-(trifluoromethyl)pyrimidin-4-yl)ethan-1-ol

To a solution of 1-[2-(trifluoromethyl)pyrimidin-4-yl]ethanone (640 mg, 3.366 mmol, 1 equiv) in MeOH (10 mL) was added NaBH₄ (382.03 mg, 10.098 mmol, 3 equiv) at 0° C. The reaction was stirred at rt for 1 h. Quenched with water (50 mL) and extracted with DCM (3×50 mL). The combined extracts were washed with water, brine, dried over Na₂SO₄, filtered and concentrated. This resulted in 1-[2-(trifluoromethyl)pyrimidin-4-yl]ethanol (400 mg, 58.75%) as a yellow oil. LCMS showed the desired product was generated and the reaction was not worked up. MS (ESI): mass calcd. for C₇H₇F₃N₂O: 192.05 m/z, found 193.00 [M+H]⁺.

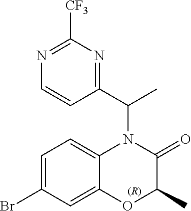

### (2R)-7-bromo-2-methyl-4-(1-(2-(trifluoromethyl)pyrimidin-4-yl)ethyl)-2H-benzo[b][1,4]oxazin-3(4H)-one

To a solution of 1-[2-(trifluoromethyl)pyrimidin-4-yl]ethanol (270 mg, 1.405 mmol, 1 equiv) in DCM (10 mL) was added (2R)-7-bromo-2-methyl-2,4-dihydro-1,4-benzoxazin-3-one (340.16 mg, 1.405 mmol, 1 equiv) and PPh₃ (552.87 mg, 2.107 mmol, 1.5 equiv). The reaction was stirred at rt under N₂ for 0.5 h. To the above mixture was added DEAD (367.09 mg, 2.107 mmol, 1.5 equiv) at 0° C. The resulting mixture was stirred for additional 1 h at rt. Quenched with water (20 mL) and extracted with DCM (3×20 mL). The combined extracts were washed with water, brine, dried over Na₂SO₄, filtered and concentrated. The residue was purified by reversed-phase flash chromatography with the following conditions: column, C18 silica gel; mobile phase, water in ACN, 65% to 75% gradient in 10 min; detector, UV 254 nm. This resulted in (2R)-7-bromo-2-methyl-4-{1-[2-(trifluoromethyl)pyrimidin-4-yl]ethyl}-2H-1,4-benzoxazin-3-one (110 mg, 18.81%) as a yellow oil. MS (ESI): mass calcd. for C₁₆H₁₃BrF₃N₃O₂: 415.01 m/z, found: 415.90, 417.90 [M+H, M+H+2]⁺.

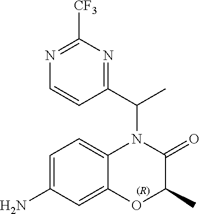

### (2R)-7-amino-2-methyl-4-(1-(2-(trifluoromethyl)pyrimidin-4-yl)ethyl)-2H-benzo[b][1,4]oxazin-3(4H)-one

To a solution of (2R)-7-bromo-2-methyl-4-{1-[2-(trifluoromethyl)pyrimidin-4-yl]ethyl}-2H-1,4-benzoxazin-3-one (180 mg, 0.432 mmol, 1 equiv) in EtOH/H₂O (4:1, 10 mL) was added (1R,2R)—N1,N2-dimethylcyclohexane-1,2-diamine (36.91 mg, 0.259 mmol, 0.6 equiv), CuSO₄ (69.02 mg, 0.432 mmol, 1 equiv), sodium ascorbate (172.23 mg, 0.864 mmol, 2 equiv) and NaN₃ (84.35 mg, 1.296 mmol, 3 equiv). The reaction was stirred at 80° C. under N₂ for 3 h. After cooling down to rt, the reaction was quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-7-amino-2-methyl-4-{1-[2-(trifluoromethyl)pyrimidin-4-yl]ethyl}-2H-1,4-benzoxazin-3-one (70 mg, 45.94%) as a yellow oil. MS (ESI): mass calcd. for C₁₆H₁₅F₃N₄O₂: 352.12 m/z, found 353.00 [M+H]⁺.

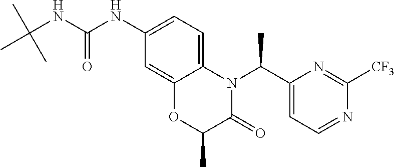 [SMILES: C[C@H]1Oc2cc(NC(=O)NC(C)(C)C)ccc2N([C@@H](C)c2ccnc(C(F)(F)F)n2)C1=O]

### 1-(tert-butyl)-3-((R)-2-methyl-3-oxo-4-((S)-1-(2-(trifluoromethyl)pyrimidin-4-yl)ethyl)-3,4-dihydro-2H-benzo[b][1,4]oxazin-7-yl)urea

To a solution of (2R)-7-amino-2-methyl-4-{1-[2-(trifluoromethyl)pyrimidin-4-yl]ethyl}-2H-1,4-benzoxazin-3-one (70 mg, 0.199 mmol, 1 equiv) in DCM/Toluene (2:1, 7.5 mL) was added 2-isocyanato-2-methylpropane (196.96 mg, 1.990 mmol, 10 equiv). The reaction was stirred at 90° C. under N₂ for 3 h. After cooling down to rt, the reaction was quenched with water (10 mL) and extracted with DCM (3×10 mL). The combined extracts were washed with water, brine, dried over Na₂SO₄, filtered and concentrated. The residue was purified by preparative HPLC using a YMC-Actus Tiart C18 ExRS Column, 30*150 mm, 5 μm column (eluent: 49% to 73% (v/v) CH₃CN and H₂O with 10 mmol/L NH₄HCO₃) to afford the title compound 3-tert-butyl-1-[(2R)-2-methyl-3-oxo-4-{1-[2-(trifluoromethyl)pyrimidin-4-yl]ethyl}-2H-1,4-benzoxazin-7-yl]urea (40 mg, 44.59%) as a white solid. MS (ESI): mass calcd. for C₂₁H₂₄F₃N₅O₃: 451.18 m/z, found 452.15 [M+H]⁺.

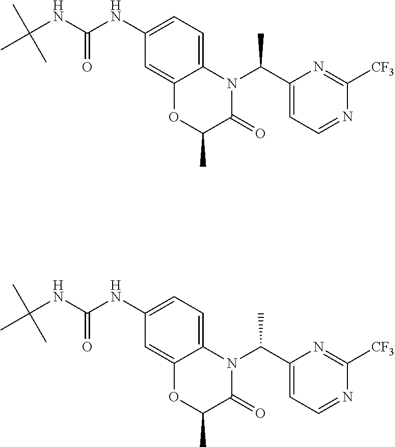 [SMILES: C[C@H]1Oc2cc(NC(=O)NC(C)(C)C)ccc2N([C@@H](C)c2ccnc(C(F)(F)F)n2)C1=O ; C[C@H](c1ccnc(C(F)(F)F)n1)N1C(=O)[C@@H](C)Oc2cc(NC(=O)NC(C)(C)C)ccc21]

### 1-(tert-butyl)-3-((R)-2-methyl-3-oxo-4-((S)-1-(2-(trifluoromethyl)pyrimidin-4-yl)ethyl)-3,4-dihydro-2H-benzo[b][1,4]oxazin-7-yl)urea and 1-(tert-butyl)-3-((R)-2-methyl-3-oxo-4-((R)-1-(2-(trifluoromethyl)pyrimidin-4-yl)ethyl)-3,4-dihydro-2H-benzo[b][1,4]oxazin-7-yl)urea

A sample of 3-tert-butyl-1-[(2R)-2-methyl-3-oxo-4-{1-[2-(trifluoromethyl)pyrimidin-4-yl]ethyl}-2H-1,4-benzoxazin-7-yl]urea (40 mg) was separated by chiral-HPLC using a CHIRALPAK IE, 2*25 cm, 5 μm column (eluent: 10% to 10% (v/v) HEX:DCM=3:1(0.5% 2M NH₃-MeOH) to yield first example 3-tert-butyl-1-[(2R)-2-methyl-3-oxo-4-[(1S)-1-[2-(trifluoromethyl)pyrimidin-4-yl]ethyl]-2H-1,4-benzoxazin-7-yl]urea (12.8 mg, 32.00%) as a white solid. MS(ESI): mass calcd. for C₂₁H₂₄F₃N₅O₃: 451.18 m/z, found: 474.15 [M+Na]⁺. ¹H NMR (400 MHz, DMSO-d₆) δ 8.81-9.10 (m, 1H), 8.26 (s, 1H), 7.59-7.90 (m, 1H), 7.16-7.34 (m, 1H), 6.58-7.03 (m, 2H), 5.78-6.01 (m, 2H), 4.65-4.80 (m, 1H), 1.78 (d, J=6.9 Hz, 3H), 1.39 (d, J=6.7 Hz, 3H), 1.27 (s, 9H). ¹⁹F NMR (376 MHz, DMSO) δ(ppm): −69.1379. And to yield second example 3-tert-butyl-1-[(2R)-2-methyl-3-oxo-4-[(1R)-1-[2-(trifluoromethyl)pyrimidin-4-yl]ethyl]-2H-1,4-benzoxazin-7-yl]urea (13.7 mg, 34.25%) as a white solid. MS(ESI): mass calcd. for C₂₁H₂₄F₃N₅O₃: 451.18 m/z, found: 452.05 [M+H]⁺. ¹H NMR (400 MHz, DMSO-d₆) δ 8.90-9.10 (m, 1H), 8.26 (s, 1H), 7.63-7.94 (m, 1H), 7.15-7.36 (m, 1H), 6.65-7.00 (m, 2H), 5.98 (m, 1H), 5.80-5.95 (m, 1H), 4.68-4.72 (m, 1H), 1.70-1.86 (m, 3H), 1.33-1.45 (m, 3H), 1.27 (s, 9H). ¹⁹F NMR (376 MHz, DMSO) δ(ppm): −69.1481.

### Example 312: 1-(tert-butyl)-3-(1-(1-(2-fluoro-3-(trifluoromethoxy)phenyl)ethyl)-2-oxo-1,2-dihydroquinoxalin-6-yl)urea

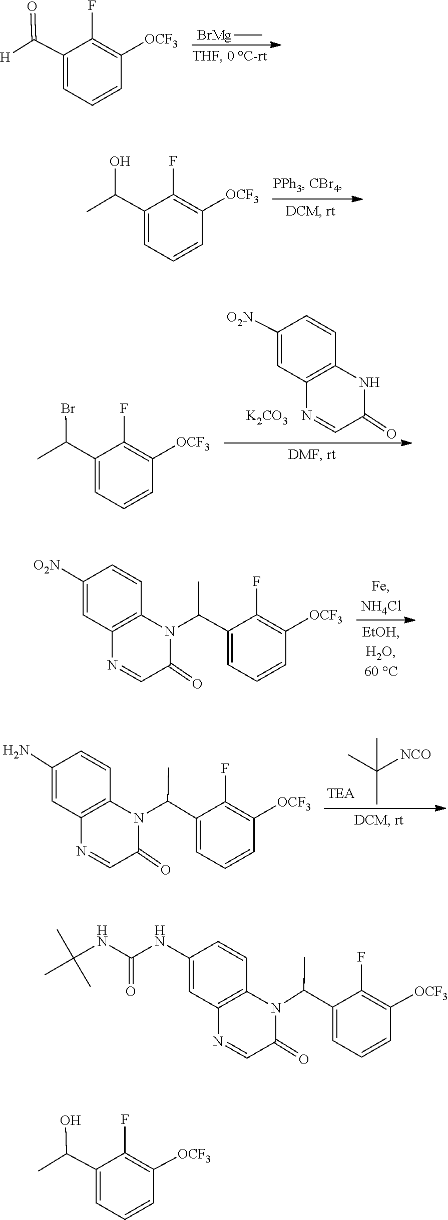

### 1-(2-fluoro-3-(trifluoromethoxy)phenyl)ethan-1-ol

To a stirring solution 2-fluoro-3-(trifluoromethoxy)benzaldehyde (500 mg, 2.403 mmol, 1 equiv) in tetrahydrofuran (10 mL) was added a solution of methylmagnesium bromide (2 M in THF, 1.8 mL, 3.604 mmol, 1.5 equiv) at 0° C. The mixture was stirred at room temperature for 5 h. The reaction mixture was quenched by water (10 mL) and extracted with EA (3×30 mL). The combined organic extracts were washed with brine (20 mL), dried over anhydrous Na₂SO₄ and concentrated under vacuum. The crude was purified by silica gel column with PE/EA (4:1) to afford 1-(2-fluoro-3-(trifluoromethoxy)phenyl)ethan-1-ol (500 mg, 92.84%) as a yellow solid.

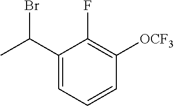

### 1-(1-bromoethyl)-2-fluoro-3-(trifluoromethoxy)benzene

To a stirring solution 1-(2-fluoro-3-(trifluoromethoxy)phenyl)ethan-1-ol (100 mg, 0.446 mmol, 1 equiv) in DCM (2 mL) was added a solution of CBr₄ (163 mg, 0.491 mmol, 1.1 equiv) in 0° C. and PPh₃ (129 mg, 0.491 mmol, 1.1 equiv) at 0° C. The mixture was stirred at room temperature for 5 h. The reaction mixture was quenched by water (10 mL) and extracted with EA (3×30 mL). The combined organic extracts were washed with brine (20 mL), dried over anhydrous Na₂SO₄, and concentrated under vacuum. The crude was purified by silica gel column with PE/EA (5:1) to afford 1-(1-bromoethyl)-2-fluoro-3-(trifluoromethoxy)benzene (100 mg, 78.09%) as a yellow solid.

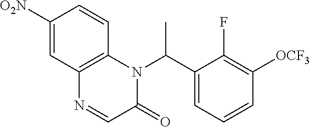 [SMILES: CC(c1cccc(OC(F)(F)F)c1F)n1c(=O)cnc2cc([N+](=O)[O-])ccc21]

### 1-(1-(2-fluoro-3-(trifluoromethoxy)phenyl)ethyl)-6-nitroquinoxalin-2(1H)-one

To a stirring solution 6-nitroquinoxalin-2(1H)-one (67 mg, 0.348 mmol, 1 equiv) in DMF (5 mL) was added K₂CO₃ (146 mg, 1.044 mmol, 3 equiv) and 1-(1-bromoethyl)-2-fluoro-3-(trifluoromethoxy)benzene (100 mg, 0.348 mmol, 1 equiv). The mixture was stirred at room temperature for 5 h. The reaction mixture was quenched by water (10 mL) and extracted with EA (3×20 mL). The combined organic extracts were washed with brine (10 mL), dried over anhydrous Na₂SO₄ and concentrated under vacuum. The crude was purified by silica gel column with PE/EA (3:1) to afford 1-(1-(2-fluoro-3-(trifluoromethoxy)phenyl)ethyl)-6-nitroquinoxalin-2(1H)-one (40 mg, 28.90%) as a yellow solid. MS (ESI): mass calcd. for C₁₇H₁₁F₄N₃O₄: 397.07 found: 398.10 [M+H]⁺.

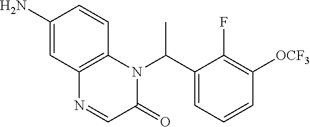 [SMILES: CC(c1cccc(OC(F)(F)F)c1F)n1c(=O)cnc2cc(N)ccc21]

### 6-amino-1-(1-(2-fluoro-3-(trifluoromethoxy)phenyl)ethyl)quinoxalin-2(1H)-one

To a stirring solution 1-(1-(2-fluoro-3-(trifluoromethoxy)phenyl)ethyl)-6-nitroquinoxalin-2(1H)-one (40 mg, 0.101 mmol, 1 equiv) in EtOH (2 mL) and H₂O (0.5 mL) was added Fe (27 mg, 0.479 mmol, 4.74 equiv) and NH₄C₁ (27 mg, 0.500 mmol, 4.95 equiv). The mixture was stirred for 2 h at 60° C. The reaction mixture was quenched by water (10 mL) and extracted with EA (3×30 mL). The combined organic extracts were washed with brine (10 mL), dried over anhydrous Na₂SO₄ and concentrated under vacuum. The crude was purified by silica gel column with PE/EA (1:1) to afford 6-amino-1-(1-(2-fluoro-3-(trifluoromethoxy)phenyl)ethyl)quinoxalin-2(1H)-one (30 mg, 81.12%) as a yellow solid. MS (ESI): mass calcd. for C₁₇H₁₃F₄N₃O₂: 367.07 found: 368.10 [M+H]⁺.

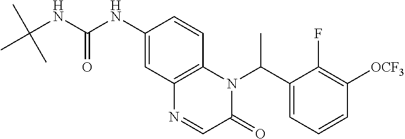 [SMILES: CC(c1cccc(OC(F)(F)F)c1F)n1c(=O)cnc2cc(NC(=O)NC(C)(C)C)ccc21]

### 1-(tert-butyl)-3-(1-(1-(2-fluoro-3-(trifluoromethoxy)phenyl)ethyl)-2-oxo-1,2-dihydroquinoxalin-6-yl)urea

To a stirring solution 6-amino-1-(1-(2-fluoro-3-(trifluoromethoxy)phenyl)ethyl)quinoxalin-2(1H)-one (30 mg, 0.082 mmol, 1 equiv) in DCM (2 mL) was added 2-isocyanato-2-methylpropane (30 mg, 0.303 mmol, 3.71 equiv) and TEA (40 mg, 0.395 mmol, 4.84 equiv). The mixture was stirred at room temperature for 16 h. The reaction mixture was quenched by water (10 mL) and extracted with DCM (3×10 mL). The combined organic extracts were washed with brine (10 mL), dried over anhydrous Na₂SO₄ and concentrated under vacuum. The crude was purified by silica gel column with PE/EA (1:1) and Prep-HPLC (Column: XBridge Prep OBD C18, 30*150 mm, 5.0 m; Mobile Phase A: Water (10 mmol/L NH₄HCO₃), Mobile Phase B:ACN; Flow rate: 60 mL/min; Gradient: 30% B to 60% B in 7 min; Wave Length: 254/220 nm; RT1(min): 10.7; Number Of Runs: 3) to afford 1-(tert-butyl)-3-(1-(1-(2-fluoro-3-(trifluoromethoxy)phenyl)ethyl)-2-oxo-1,2-dihydroquinoxalin-6-yl)urea (9.5 mg, 24.79%) as a yellow solid. MS (ESI): mass calcd. for C₂₂H₂₂F₄N₄O₃: 466.15 found: 467.00[M+H]⁺. ¹H NMR (400 MHz, DMSO-d₆) δ 8.45 (s, 1H), 8.14 (s, 1H), 7.91 (d, J=2.4 Hz, 1H), 7.73 (t, J=7.4 Hz, 1H), 7.44-7.54 (m, 3H), 7.37 (t, J=8.2 Hz, 1H), 6.54 (s, 1H), 6.04 (s, 1H), 1.89 (d, J=7.0 Hz, 3H), 1.29 (s, 9H).

### Example 313: (S)-1-(tert-butyl)-3-(2-oxo-1-(1-(3-(2,2,2-trifluoroethoxy)phenyl)ethyl)-1,2-dihydroquinoxalin-6-yl)urea

### Synthetic Scheme

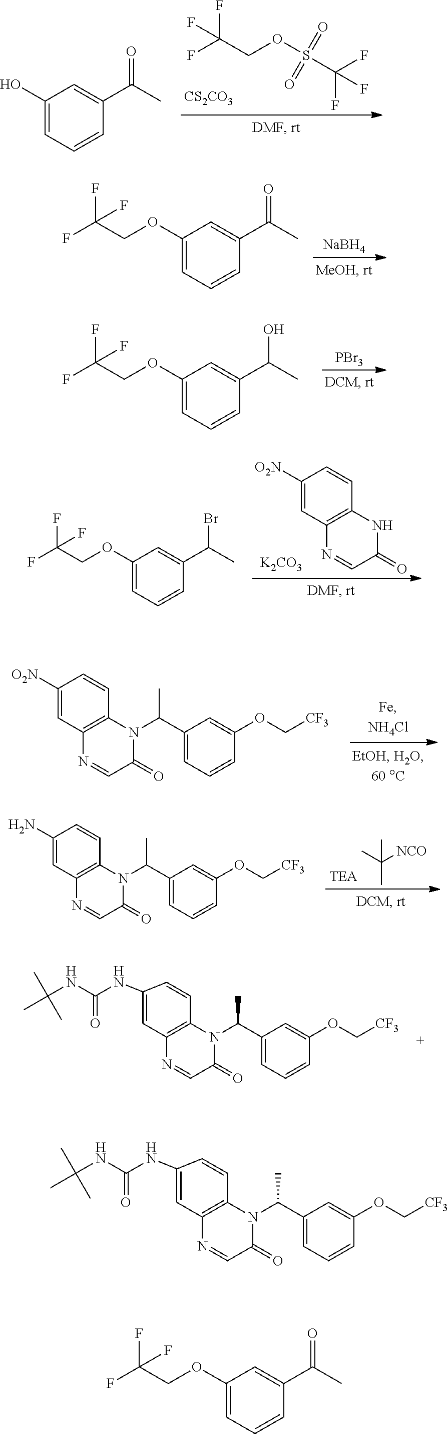

### 1-(3-(2,2,2-trifluoroethoxy)phenyl)ethan-1-one

To a stirred solution of 3-hydroxyacetophenone (1.5 g, 11.017 mmol, 1 equiv) was added dimethylformamide (15 mL) followed by catalytic amount of 2,2,2-trifluoroethyl trifluoromethanesulfonate (3.84 g, 16.526 mmol, 1.5 equiv) and at room temperature. The reaction mixture was stirred at room temperature for a period of 3 h. After completion of reaction, the reaction mixture was quenched by water (30 mL) and extracted with EA (3×30 mL). The combined organic extracts were washed with brine (30 mL), dried over anhydrous Na₂SO₄, and concentrated under vacuum to yield a crude product which was directly purified by silica gel column, eluted with PE/EA (2:1) to afford desired 1-(3-(2,2,2-trifluoroethoxy)phenyl)ethan-1-one (1.5 g, 62.40%). MS (ESI): mass calcd. for C₁₀H₉F₃O₂: 218.175 m/z, found: 219.15 [M+H]⁺.

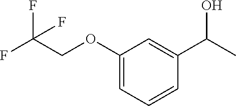

### 1-(3-(2,2,2-trifluoroethoxy)phenyl)ethan-1-ol

To a solution of 1-(3-(2,2,2-trifluoroethoxy)phenyl)ethan-1-one (2.2 g, 10.249 mmol, 1 equiv) in methanol (30 mL) was added NaBH₄ (0.77 g, 20.498 mmol, 2 equiv)) at 0° C. The mixture was stirred for 30 min. The reaction mixture was quenched by water (20 mL) and extracted with EA (3×100 mL). Then, the combined organic phase was washed with brine (20 mL), dried over anhydrous Na₂SO₄, and concentrated under vacuum to yield a crude product which was directly purified by silica gel column, eluted with PE/EA (1:1) to afford 1-(3-(2,2,2-trifluoroethoxy)phenyl)ethan-1-ol (1.23 g, 54.68%) as a yellow oil.

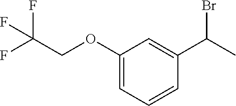

### 1-(1-bromoethyl)-3-(2,2,2-trifluoroethoxy)benzene

To a solution of 1-(3-(2,2,2-trifluoroethoxy)phenyl)ethan-1-ol (1.0 g, 4.542 mmol, 1 equiv) in methylene chloride (20 mL) the mixture was 0° C. then added phosphorus tribromide (6.15 g, 22.710 mmol, 5.0 equiv) slowly, the mixture was warmed to room temperature stirred at room temperature for 3 h. The reaction mixture was quenched by ice-water and extracted with EA (3×50 mL). The combined organic extracts were washed with brine (20 mL), dried over anhydrous Na₂SO₄, and concentrated under vacuum to yield a crude product which was directly purified by silica gel column, eluted with PE/EA (2:1) to afford 1-(1-bromoethyl)-3-(2,2,2-trifluoroethoxy)benzene (725 mg, 56.39%) as a yellow oil.

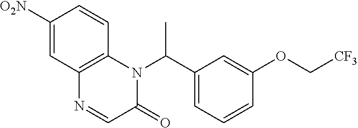 [SMILES: CC(c1cccc(OCC(F)(F)F)c1)n1c(=O)cnc2cc([N+](=O)[O-])ccc21]

### 6-nitro-1-(1-(3-(2,2,2-trifluoroethoxy)phenyl)ethyl)quinoxalin-2(1H)-one

To a solution of 1-(1-bromoethyl)-3-(2,2,2-trifluoroethoxy)benzene (430 mg, 1.519 mmol, 1 equiv) in dimethylformamide (10 mL) was added potassium methaneperoxoate potassium (634 mg, 4.557 mmol, 3 equiv) at room temperature. Then 6-nitro-1H-quinoxalin-2-one (290 mg, 1.519 mmol, 1 equiv) was added at room temperature. The reaction was stirred at room temperature for 6 h. The reaction mixture was quenched by water and extracted with EA (3×100 mL). The combined organic extracts were washed with brine (30 mL), dried over anhydrous Na₂SO₄, and concentrated under vacuum to yield a crude product which was directly purified by silica gel column, eluted with PE/EA (1:1) to afford 6-nitro-1-(1-(3-(2,2,2-trifluoroethoxy)phenyl)ethyl)quinoxalin-2(1H)-one (380 mg, 63.60%). MS (ESI): mass calcd. for C18H14F3N3O4: 393.322 m/z, found: 394.20 [M+H]⁺.

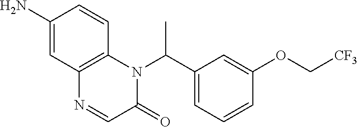 [SMILES: CC(c1cccc(OCC(F)(F)F)c1)n1c(=O)cnc2cc(N)ccc21]

### 6-amino-1-(1-(3-(2,2,2-trifluoroethoxy)phenyl)ethyl)quinoxalin-2(1H)-one

To a stirred solution of 6-nitro-1-(1-(3-(2,2,2-trifluoroethoxy)phenyl)ethyl)quinoxalin-2(1H)-one (50 mg, 0.127 mmol, 1 equiv) in methanol (10 mL) and water (2 mL) was added iron (71 mg, 1.27 mmol, 10 equiv) followed by catalytic amount of NH₄C₁ (67 mg, 1.27 mmol, 10 equiv) at room temperature. The reaction mixture was stirred at 60° C. for a period of 0.5 h. The reaction mixture was quenched by water (20 mL) and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (1:1) to afford desired 6-amino-1-(1-(3-(2,2,2-trifluoroethoxy)phenyl)ethyl)quinoxalin-2(1H)-one (30 mg, 64.93%). MS (ESI): mass calcd. for C₁₈H₁₆F₃N₃O₂: 363.34 m/z, found: 364.20 [M+H]⁺.

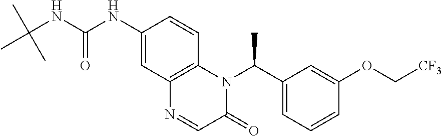 [SMILES: C[C@@H](c1cccc(OCC(F)(F)F)c1)n1c(=O)cnc2cc(NC(=O)NC(C)(C)C)ccc21]

### (S)-1-(tert-butyl)-3-(2-oxo-1-(1-(3-(2,2,2-trifluoroethoxy)phenyl)ethyl)-1,2-dihydroquinoxalin-6-yl)urea

To a stirring solution 6-amino-1-(1-(3-(2,2,2-trifluoroethoxy)phenyl)ethyl)quinoxalin-2(1H)-one (200 mg, 0.550 mmol, 1 equiv) in DCM (5 mL) was added a solution of 2-isocyanato-2-methylpropane (272 mg, 2.750 mmol, 5 equiv) and TEA (557 mg, 5.500 mmol, 10 equiv). The mixture was stirred at room temperature for 16 h. The mixture was concentrated to dryness under reduced pressure to provide the crude product, which was purified by C18 column (eluent: CH₃CN and H₂O), Prep-HPLC (Column: XBridge Prep OBD C18, 30*150 mm, 5.0 μm; Mobile Phase A: Water (10 mmol/L NH₄HCO₃), Mobile Phase B:ACN; Flow rate: 60 mL/min; Gradient: 30% B to 60% B in 7 min) and Chiral-HPLC (Column: (R, R)-WHELK-01-Kromasil 5*25 cm, 5 μm; Mobile Phase A: Hex(0.5% 2M NH₃-MeOH)—HPLC, Mobile Phase B: EtOH—HPLC; Flow rate: 20 mL/min; Gradient: isocratic 30; Wave Length: 220/254 nm; RT1(min): 21.321; RT2(min): 28.591; Injection Volume: 0.9 mL; Number Of Runs: 5) to afford (S)-1-(tert-butyl)-3-(2-oxo-1-(1-(3-(2,2,2-trifluoroethoxy)phenyl)ethyl)-1,2-dihydroquinoxalin-6-yl)urea (8.2 mg, 3.21%) as a yellow solid. MS (ESI): mass calcd. for C₂₃H₂₅F₃N₄O₃: 462.18 m/z, found: 463.15 [M+H]⁺. ¹H NMR (300 MHz, DMSO-d₆) δ 8.41 (s, 1H), 8.26 (s, 1H), 7.93 (d, J=2.5 Hz, 1H), 7.23-7.36 (m, 2H), 7.05 (br, 1H), 6.94-7.01 (m, 2H), 6.90 (d, J=7.8 Hz, 1H), 6.57 (br, 1H), 6.03 (s, 1H), 4.76 (q, J=8.9 Hz, 2H), 1.87 (d, J=7.1 Hz, 3H), 1.28 (s, 9H). And another isomer (R)-1-(tert-butyl)-3-(2-oxo-1-(1-(3-(2,2,2-trifluoroethoxy)phenyl)ethyl)-1,2-dihydroquinoxalin-6-yl)urea as a yellow solid (6 mg, 2.35%) as a yellow solid. MS (ESI): mass calcd. for C₂₃H₂₅F₃N₄O₃: 462.18 m/z, found: 463.15 [M+H]⁺. ¹H NMR (300 MHz, DMSO-d₆) δ 8.41 (s, 1H), 8.26 (s, 1H), 7.93 (d, J=2.5 Hz, 1H), 7.23-7.36 (m, 2H), 7.05 (br, 1H), 6.94-7.01 (m, 2H), 6.90 (d, J=7.8 Hz, 1H), 6.57 (br, 1H), 6.03 (s, 1H), 4.76 (q, J=8.9 Hz, 2H), 1.87 (d, J=7.1 Hz, 3H), 1.28 (s, 9H).

### Example 314: (R)-1-(tert-butyl)-3-(2-oxo-1-(1-(3-(2,2,2-trifluoroethoxy)phenyl)ethyl)-1,2-dihydroquinoxalin-6-yl)urea

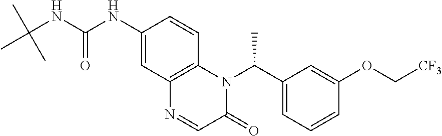 [SMILES: C[C@H](c1cccc(OCC(F)(F)F)c1)n1c(=O)cnc2cc(NC(=O)NC(C)(C)C)ccc21]

### (R)-1-(tert-butyl)-3-(2-oxo-1-(1-(3-(2,2,2-trifluoroethoxy)phenyl)ethyl)-1,2-dihydroquinoxalin-6-yl)urea

To a stirring solution 6-amino-1-(1-(3-(2,2,2-trifluoroethoxy)phenyl)ethyl)quinoxalin-2(1H)-one (200 mg, 0.550 mmol, 1 equiv) in DCM (5 mL) was added a solution of 2-isocyanato-2-methylpropane (272 mg, 2.750 mmol, 5 equiv) and TEA (557 mg, 5.500 mmol, 10 equiv). The mixture was stirred at room temperature for 16 h. The mixture was concentrated to dryness under reduced pressure to provide the crude product, which was purified by C18 column (eluent: CH₃CN and H₂O), Prep-HPLC (Column: XBridge Prep OBD C18, 30*150 mm, 5.0 μm; Mobile Phase A: Water (10 mmol/L NH₄HCO₃), Mobile Phase B:ACN; Flow rate: 60 mL/min; Gradient: 30% B to 60% B in 7 min) and Chiral-HPLC (Column: (R, R)-WHELK-01-Kromasil 5*25 cm, 5 μm; Mobile Phase A: Hex(0.5% 2M NH₃-MeOH)—HPLC, Mobile Phase B: EtOH—HPLC; Flow rate: 20 mL/min; Gradient: isocratic 30; Wave Length: 220/254 nm; RT1(min): 21.321; RT2(min): 28.591; Injection Volume: 0.9 mL; Number Of Runs: 5) to afford (S)-1-(tert-butyl)-3-(2-oxo-1-(1-(3-(2,2,2-trifluoroethoxy)phenyl)ethyl)-1,2-dihydroquinoxalin-6-yl)urea (8.2 mg, 3.21%) as a yellow solid. MS (ESI): mass calcd. for C₂₃H₂₅F₃N₄O₃: 462.18 m/z, found: 463.15 [M+H]⁺. ¹H NMR (300 MHz, DMSO-d₆) δ 8.41 (s, 1H), 8.26 (s, 1H), 7.93 (d, J=2.5 Hz, 1H), 7.23-7.36 (m, 2H), 7.05 (br, 1H), 6.94-7.01 (m, 2H), 6.90 (d, J=7.8 Hz, 1H), 6.57 (br, 1H), 6.03 (s, 1H), 4.76 (q, J=8.9 Hz, 2H), 1.87 (d, J=7.1 Hz, 3H), 1.28 (s, 9H). And another isomer (R)-1-(tert-butyl)-3-(2-oxo-1-(1-(3-(2,2,2-trifluoroethoxy)phenyl)ethyl)-1,2-dihydroquinoxalin-6-yl)urea as a yellow solid (6 mg, 2.35%) as a yellow solid. MS (ESI): mass calcd. for C₂₃H₂₅F₃N₄O₃: 462.18 m/z, found: 463.15 [M+H]+. ¹H NMR (300 MHz, DMSO-d₆) δ 8.41 (s, 1H), 8.26 (s, 1H), 7.93 (d, J=2.5 Hz, 1H), 7.23-7.36 (m, 2H), 7.05 (br, 1H), 6.94-7.01 (m, 2H), 6.90 (d, J=7.8 Hz, 1H), 6.57 (br, 1H), 6.03 (s, 1H), 4.76 (q, J=8.9 Hz, 2H), 1.87 (d, J=7.1 Hz, 3H), 1.28 (s, 9H).

### Example 315: (S)—N-(2-oxo-1-(1-(3-(trifluoromethoxy)phenyl)ethyl)-1,2-dihydroquinoxalin-6-yl)cyclopentanecarboxamide

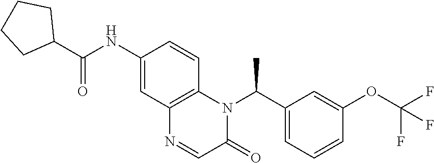 [SMILES: C[C@@H](c1cccc(OC(F)(F)F)c1)n1c(=O)cnc2cc(NC(=O)C3CCCC3)ccc21]

### (S)—N-(2-oxo-1-(1-(3-(trifluoromethoxy)phenyl)ethyl)-1,2-dihydroquinoxalin-6-yl)cyclopentanecarboxamide

To a stirring solution 6-amino-1-[(1S)-1-[3-(trifluoromethoxy)phenyl]ethyl]quinoxalin-2-one (40 mg, 0.115 mmol, 1 equiv) in pyridine (3 mL) was added a solution of cyclopentanecarboxylic acid (16.99 mg, 0.150 mmol, 1.3 equiv) and EDCI (33 mg, 0.173 mmol, 1.5 equiv). The mixture was stirred at room temperature for 16 h. The mixture was concentrated and purified by C18 column (eluent: CH₃CN and H₂O) to afford (S)—N-(2-oxo-1-(1-(3-(trifluoromethoxy)phenyl)ethyl)-1,2-dihydroquinoxalin-6-yl)cyclopentanecarboxamide (32.3 mg, 19.32%) as a yellow solid. MS (ESI): mass calcd. for: C₂₃H₂₂F₃N₃O₃: 445.44 m/z, found: 446.10 [M+H]⁺. ¹H NMR (300 MHz, DMSO-d₆) δ 10.07 (s, 1H), 8.27 (s, 1H), 8.19 (d, J=2.5 Hz, 1H), 7.59 (d, J=9.1 Hz, 1H), 7.47 (t, J=8.1 Hz, 1H), 7.30 (d, J=4.9 Hz, 4H), 7.17 (s, 1H), 6.58 (s, 1H), 2.74-2.79 (m, 1H), 1.83-1.88 (m, 5H), 1.56-1.68 (m, 4H), 1.45-1.53 (m, 2H).

### Example 316 and Example 317: 1-(tert-butyl)-3-((R)-2-methyl-3-oxo-4-((S)-1-(4-(trifluoromethyl)pyrimidin-2-yl)ethyl)-3,4-dihydro-2H-benzo[b][1,4]oxazin-7-yl)urea and 1-(tert-butyl)-3-((R)-2-methyl-3-oxo-4-((R)-1-(4-(trifluoromethyl)pyrimidin-2-yl)ethyl)-3,4-dihydro-2H-benzo[b][1,4]oxazin-7-yl)urea

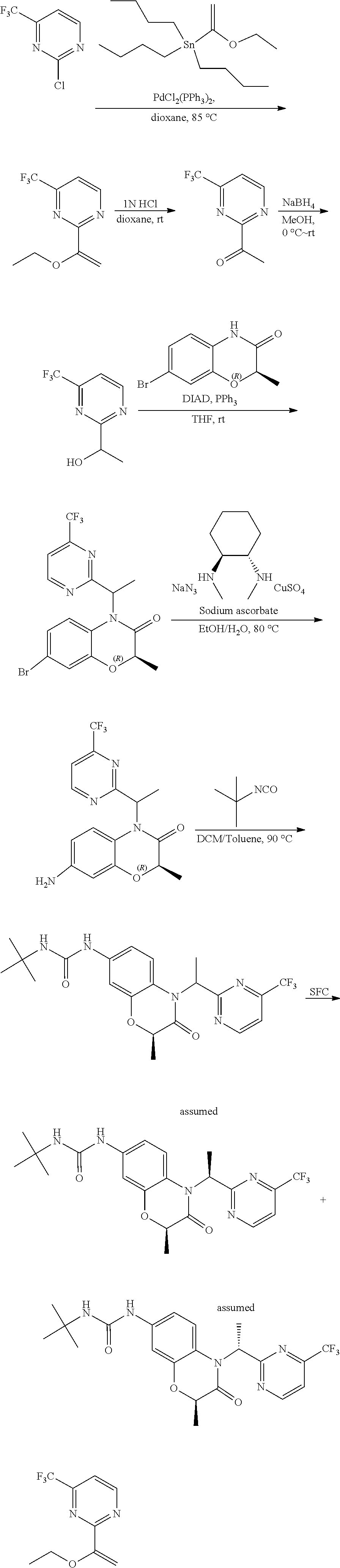

### 2-(1-ethoxyvinyl)-4-(trifluoromethyl)pyrimidine

To a solution of 2-chloro-4-(trifluoromethyl)pyrimidine (5 g, 27.393 mmol, 1 equiv) in dioxane (70 mL) was added tributyl(1-ethoxyethenyl)stannane (14.84 g, 41.090 mmol, 1.5 equiv) and bis(triphenylphosphane) dichloropalladium (1.92 g, 2.739 mmol, 0.1 equiv). The resulting mixture was maintained under nitrogen and stirred at 90° C. for 3 h. After cooling down to rt, the reaction was quenched with water (150 mL). The resulting mixture was extracted with ethyl acetate (3×150 mL). The organic layers were combined, dried over anhydrous sodium sulfate, filtered and concentrated. The residue obtained was purified by silica gel chromatography (050 ethyl acetate/petroleum ether) to afford the 2-(1-ethoxyvinyl)-4-(trifluoromethyl)pyrimidine (5 g, 83.66%) as a yellow solid. LC/MS: mass calcd. for C₈H₈F₃N₂O: 218.07 m/z, found: 219.00 [M+H]⁺.

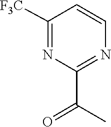

### 1-(4-(trifluoromethyl)pyrimidin-2-yl)ethan-1-one

To a solution of 2-(1-ethoxyvinyl)-4-(trifluoromethyl)pyrimidine (1.5 g, 6.875 mmol, 1 equiv) in dioxane (70 mL) was added 1N HCl (15 mL). The resulting mixture was maintained under air and stirred at rt for 3 h. The reaction was quenched with water (100 mL). The resulting mixture was extracted with ethyl acetate (3×100 mL). The organic layers were combined, dried over anhydrous sodium sulfate, filtered and concentrated. The residue obtained was purified by silica gel chromatography (0-50% ethyl acetate/petroleum ether) to afford the 1-(4-(trifluoromethyl)pyrimidin-2-yl)ethan-1-one (800 mg, 61.20%) as a yellow oil. LC/MS: mass calcd. for C₇H₅F₃N₂O: 190.04 m/z, found: 191.10 [M+H]⁺.

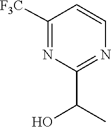

### 1-(4-(trifluoromethyl)pyrimidin-2-yl)ethan-1-ol

To a solution of 1-(4-(trifluoromethyl)pyrimidin-2-yl)ethan-1-one (2.2 g, 11.571 mmol, 1 equiv) in MeOH (24 mL) was added NaBH₄ (656.62 mg, 17.357 mmol, 1.5 equiv) at 0° C. The resulting mixture was maintained under air and stirred at rt for 3 h. The reaction was quenched with water (100 mL). The resulting mixture was extracted with ethyl acetate (3×100 mL). The organic layers were combined, dried over anhydrous sodium sulfate, filtered and concentrated. The residue obtained was purified by silica gel chromatography (0-50% ethyl acetate/petroleum ether) to afford the 1-(4-(trifluoromethyl)pyrimidin-2-yl)ethan-1-ol (1 g, 44.98%) as a yellow oil. LC/MS: mass calcd. for C₇H₇F₃N₂O: 192.05 m/z, found: 193.00 [M+H]⁺.

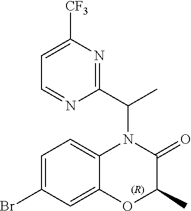

### (2R)-7-bromo-2-methyl-4-(1-(4-(trifluoromethyl)pyrimidin-2-yl)ethyl)-2H-benzo[b][1,4]oxazin-3(4H)-one

To a solution of 1-(4-(trifluoromethyl)pyrimidin-2-yl)ethan-1-ol (300 mg, 1.561 mmol, 1 equiv) in THF (5 mL) was added (R)-7-bromo-2-methyl-2H-benzo[b][1,4]oxazin-3(4H)-one (377.96 mg, 1.561 mmol, 1 equiv) and PPh₃ (614.30 mg, 2.341 mmol, 1.5 equiv). The reaction was stirred at rt under N₂ for 0.5 h. To the above mixture was added DIAD (473.58 mg, 2.341 mmol, 1.5 equiv) at 0° C. The resulting mixture was stirred for additional 1 h at rt. The reaction was quenched with water (30 mL). The resulting mixture was extracted with ethyl acetate (3×30 mL). The organic layers were combined, dried over anhydrous sodium sulfate, filtered and concentrated. The residue obtained was purified by silica gel chromatography (0-50% ethyl acetate/petroleum ether) to afford the (2R)-7-bromo-2-methyl-4-(1-(4-(trifluoromethyl)pyrimidin-2-yl)ethyl)-2H-benzo[b][1,4]oxazin-3(4H)-one (550 mg, 84.64%) as a yellow oil. LC/MS: mass calcd. for C₁₆H₁₃BrF₃N₃O₂: 415.01 m/z, found: 416.00, 418.00 [M+H, M+H+2]⁺.

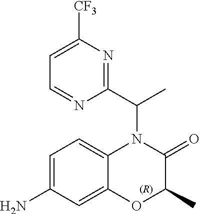

### (2R)-7-amino-2-methyl-4-(1-(4-(trifluoromethyl)pyrimidin-2-yl)ethyl)-2H-benzo[b][1,4]oxazin-3(4H)-one

To a solution of 6-nitro-1-[(1S)-1-[6-(trifluoromethoxy)pyridin-2-yl]ethyl]quinoxalin-2-one (540 mg, 1.122 mmol, 1 equiv) in EtOH (4 mL) and H₂O (1 mL) was added (1R,2R)—N1,N2-dimethylcyclohexane-1,2-diamine (95.77 mg, 0.673 mmol, 0.6 equiv), CuSO₄ (179.10 mg, 1.122 mmol, 1 equiv), sodium ascorbate (446.88 mg, 2.244 mmol, 2 equiv) and sodium azide (218.86 mg, 3.366 mmol, 3 equiv). The reaction was stirred at 80° C. for 1 h. After cooling down to rt, the filtrate was quenched with water (30 mL) and extracted with EA (3×30 mL). The combined extracts were washed with water, brine, dried over Na₂SO₄, filtered and concentrated. The residue was purified by column chromatography on silica gel with EA/PE (0-100%) to give (2R)-7-amino-2-methyl-4-(1-(4-(trifluoromethyl)pyrimidin-2-yl)ethyl)-2H-benzo[b][1,4]oxazin-3(4H)-one (360 mg, 91.06%) as a yellow solid. MS (ESI): mass calcd. for C₁₆H₁₅F₃N₄O₂: 352.11 m/z, found 352.95 [M+H]⁺.

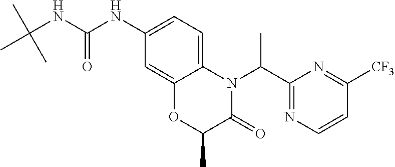 [SMILES: CC(c1nccc(C(F)(F)F)n1)N1C(=O)[C@@H](C)Oc2cc(NC(=O)NC(C)(C)C)ccc21]

### 1-(tert-butyl)-3-((2R)-2-methyl-3-oxo-4-(1-(4-(trifluoromethyl)pyrimidin-2-yl)ethyl)-3,4-dihydro-2H-benzo[b][1,4]oxazin-7-yl)urea

To a solution of (2R)-7-amino-2-methyl-4-{1-[4-(trifluoromethyl)pyrimidin-2-yl]ethyl}-2H-1,4-benzoxazin-3-one (280 mg, 0.795 mmol, 1 equiv) in DCM (10 mL) was added TEA (482.53 mg, 4.770 mmol, 6 equiv) and 2-isocyanato-2-methylpropane (787.85 mg, 7.950 mmol, 10 equiv). The reaction was stirred at rt under N₂ for 3 h. Quenched with water (50 mL) and extracted with DCM (3×50 mL). The combined extracts were washed with water, brine, dried over Na₂SO₄, filtered and concentrated. The residue was purified by preparative HPLC using a XBridge Shield RP 18 OBD Column, 30*150 mm, 5 μm column (eluent: 39% to 69% (v/v) CH₃CN and Water (10 mmol/L NH₄HCO_{3+0.1}% NH₃·H₂O)) to afford the title compound 3-tert-butyl-1-[(2R)-2-methyl-3-oxo-4-{1-[4-(trifluoromethyl)pyrimidin-2-yl]ethyl}-2H-1,4-benzoxazin-7-yl]urea (80 mg, 22.30%) as a white solid. LC/MS (ESI): mass calcd. for C₂₁H₂₄F₃N₅O₃:451.18 m/z, found: 452.15 [M+H]⁺.

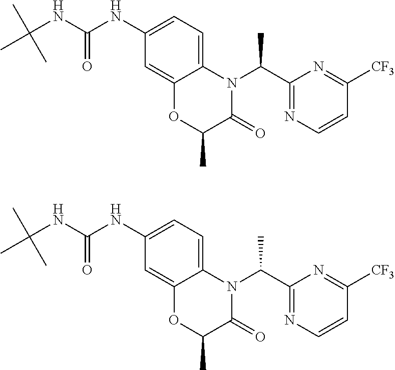 [SMILES: C[C@H]1Oc2cc(NC(=O)NC(C)(C)C)ccc2N([C@@H](C)c2nccc(C(F)(F)F)n2)C1=O ; C[C@H](c1nccc(C(F)(F)F)n1)N1C(=O)[C@@H](C)Oc2cc(NC(=O)NC(C)(C)C)ccc21]

### 1-(tert-butyl)-3-((R)-2-methyl-3-oxo-4-((S)-1-(4-(trifluoromethyl)pyrimidin-2-yl)ethyl)-3,4-dihydro-2H-benzo[b][1,4]oxazin-7-yl)urea and 1-(tert-butyl)-3-((R)-2-methyl-3-oxo-4-((R)-1-(4-(trifluoromethyl)pyrimidin-2-yl)ethyl)-3,4-dihydro-2H-benzo[b][1,4]oxazin-7-yl)urea

A sample of 3-tert-butyl-1-[(2R)-2-methyl-3-oxo-4-{1-[4-(trifluoromethyl)pyrimidin-2-yl]ethyl}-2H-1,4-benzoxazin-7-yl]urea (80 mg) was separated by chiral-HPLC using a CHIRALPAK IF 2*25 cm, 5 μm column (eluent: 25% to 25% (v/v) EtOH and Hex(0.5% 2M NH₃-MeOH) to yield first enantiomer example 3-tert-butyl-1-[(2R)-2-methyl-3-oxo-4-[(1S)-1-[4-(trifluoromethyl)pyrimidin-2-yl]ethyl]-2H-1,4-benzoxazin-7-yl]urea (29.9 mg, 37.38%) as a white solid. MS(ESI): mass calcd. for C₂₁H₂₄F₃N₅O₃: 451.18 m/z, found: 452.05 [M+H]⁺. ¹H NMR (400 MHz, DMSO-d₆) δ 9.16 (d, J=5.0 Hz, 1H), 8.25 (s, 1H), 7.91 (d, J=5.1 Hz, 1H), 7.23 (d, J=2.3 Hz, 1H), 6.88-7.00 (m, 1H), 6.70-6.88 (m, 1H), 5.96 (s, 1H), 5.81-5.13 (m, 1H), 4.61 (q, J=6.7 Hz, 1H), 1.82 (d, J=6.9 Hz, 3H), 1.34 (d, J=6.7 Hz, 3H), 1.28 (s, 9H). ¹⁹F NMR (376 MHz, DMSO) δ(ppm): −68.7349, and to yield second enantiomer example 3-tert-butyl-1-[(2R)-2-methyl-3-oxo-4-[(1R)-1-[4-(trifluoromethyl)pyrimidin-2-yl]ethyl]-2H-1,4-benzoxazin-7-yl]urea (25.9 mg, 32.38%) as a white solid. MS(ESI): mass calcd. for C₂₁H₂₄F₃N₅O₃: 451.18 m/z, found: 452.05 [M+H]⁺. ¹H NMR (400 MHz, DMSO-d₆) δ 9.16 (d, J=5.0 Hz, 1H), 8.22 (s, 1H), 7.92 (d, J=5.1 Hz, 1H), 7.23 (d, J=2.2 Hz, 1H), 6.45-7.00 (m, 2H), 5.98-6.06 (m, 1H), 5.95 (s, 1H), 4.33-4.72 (m, 1H), 1.81 (d, J=7.0 Hz, 3H), 1.36 (d, J=6.8 Hz, 3H), 1.27 (s, 9H). ¹⁹F NMR (376 MHz, DMSO) δ(ppm): −68.7105.

### Example 318: (S)—N-(2-oxo-1-(1-(6-(trifluoromethoxy)pyridin-2-yl)ethyl)-1,2-dihydroquinoxalin-6-yl)-3-(3-(trifluoromethyl)phenyl)propanamide

### Synthetic Scheme

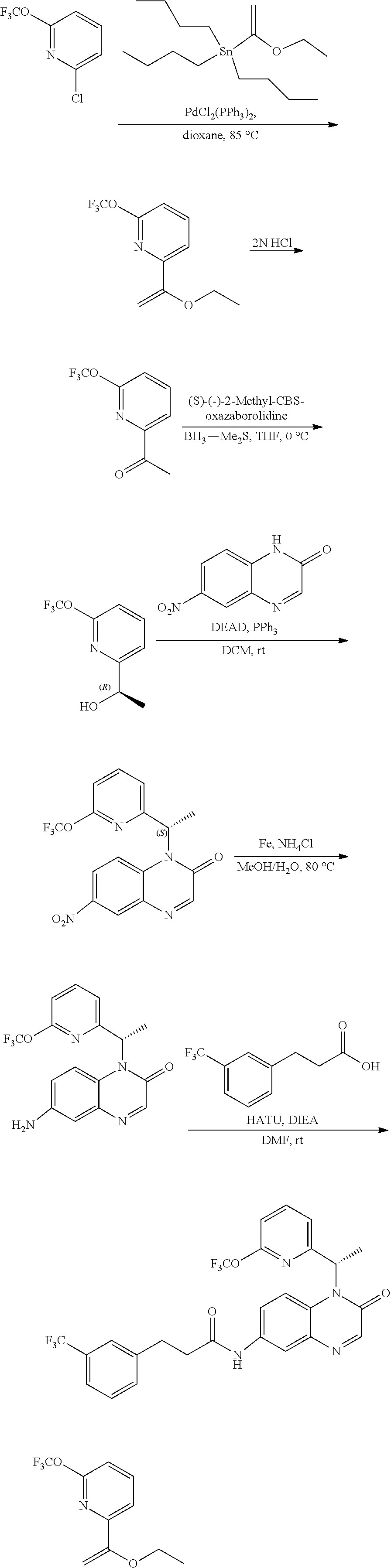

### 2-(1-ethoxyvinyl)-6-(trifluoromethoxy)pyridine

To a solution of 2-chloro-6-(trifluoromethoxy)pyridine (1 g, 5.062 mmol, 1 equiv) and Pd(PPh₃)₂C₁₂ (0.36 g, 0.506 mmol, 0.1 equiv) in dioxane (10 mL) was added tributyl(1-ethoxyethenyl)stannane (2.74 g, 7.593 mmol, 1.5 equiv) under N₂. The reaction was stirred at 85° C. under N₂ for 1 h. After cooling down to rt, the reaction was quenched with water (50 mL) and extracted with EA (3×50 mL). The combined extracts were washed with water, brine, dried over Na₂SO₄, filtered and concentrated. This resulted in 2-(1-ethoxyethenyl)-6-(trifluoromethoxy)pyridine (1 g, 84.71%) as a white oil. MS (ESI): mass calcd. for C₁₀H₁₀F₃NO₂: 233.07 m/z, found 233.95 [M+H]⁺.

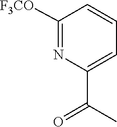

### 1-(6-(trifluoromethoxy)pyridin-2-yl)ethan-1-one

To a solution of 2-(1-ethoxyethenyl)-6-(trifluoromethoxy)pyridine (1 g, 4.288 mmol, 1 equiv) in dioxane (10 mL) was added 2N HCl (5 mL). The reaction was stirred at rt for 1 day. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 1-[6-(trifluoromethoxy)pyridin-2-yl]ethanone (0.75 g, 75.26%) as a white oil. MS (ESI): mass calcd. for C₈H₆F₃NO₂: 205.04 m/z, found 205.95 [M+H]⁺.

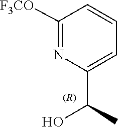

### (R)-1-(6-(trifluoromethoxy)pyridin-2-yl)ethan-1-ol

To a solution of (3aS)-1-methyl-3,3-diphenyl-hexahydropyrrolo[1,2-c][1,3,2]oxazaborole (162.14 mg, 0.585 mmol, 0.2 equiv) in THF (10 mL) was added BH₃-Me₂S (333.26 mg, 4.387 mmol, 1.5 equiv) and 1-[6-(trifluoromethoxy)pyridin-2-yl]ethanone (600 mg, 2.925 mmol, 1 equiv) at 0° C.

The reaction was stirred at 0° C. for 1 h. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (1R)-1-[6-(trifluoromethoxy)pyridin-2-yl]ethanol (260 mg, 42.91%) as a white oil. MS (ESI): mass calcd. for C₈H₈F₃NO₂: 207.05 m/z, found: 208.10 [M+H]⁺.

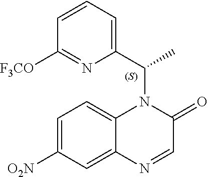

### (S)-6-nitro-1-(1-(6-(trifluoromethoxy)pyridin-2-yl)ethyl)quinoxalin-2(1H)-one

To a solution of (1R)-1-[6-(trifluoromethoxy)pyridin-2-yl]ethanol (240 mg, 1.159 mmol, 1 equiv) in DCM (10 mL) was added 6-nitro-1H-quinoxalin-2-one (221.46 mg, 1.159 mmol, 1 equiv) and PPh₃ (455.83 mg, 1.739 mmol, 1.5 equiv). The reaction was stirred at rt under N₂ for 0.5 h. To the above mixture was added DEAD (302.66 mg, 1.739 mmol, 1.5 equiv) at 0° C. The resulting mixture was stirred for additional 1 h at rt. Quenched with water (20 mL) and extracted with DCM (3×20 mL). The combined extracts were washed with water, brine, dried over Na₂SO₄, filtered and concentrated. The residue was purified by column chromatography on silica gel with EA/PE (0-100%) to give 6-nitro-1-[(1S)-1-[6-(trifluoromethoxy)pyridin-2-yl]ethyl]quinoxalin-2-one (100 mg, 22.70%) as a yellow oil. MS (ESI): mass calcd. for C₁₆H₁₁F₃N₄O₄: 380.07 m/z, found 380.95 [M+H]⁺.

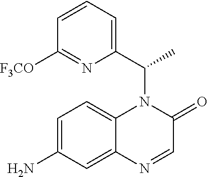 [SMILES: C[C@@H](c1cccc(OC(F)(F)F)n1)n1c(=O)cnc2cc(N)ccc21]

### (S)-6-amino-1-(1-(6-(trifluoromethoxy)pyridin-2-yl)ethyl)quinoxalin-2(1H)-one

To a solution of 6-nitro-1-[(1S)-1-[6-(trifluoromethoxy)pyridin-2-yl]ethyl]quinoxalin-2-one (1 g, 2.630 mmol, 1 equiv) in MeOH/H₂O (10:1, 11 mL) was added Fe (1.47 g, 26.300 mmol, 10 equiv) and NH₄C₁ (1.41 g, 26.300 mmol, 10 equiv). The reaction was stirred at 80° C. for 1 h. The mixture was allowed to cool down to rt. the resulting mixture was filtered, the filter cake was washed with MeOH (3×20 mL). The filtrate was concentrated under reduced pressure. Diluted with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 6-amino-1-[(1S)-1-[6-(trifluoromethoxy)pyridin-2-yl]ethyl]quinoxalin-2-one (460 mg, 49.94%) as a yellow solid. MS (ESI): mass calcd. for C₁₆H₁₃F₃N₄O₂: 350.10 m/z, found 351.00 [M+H]⁺.

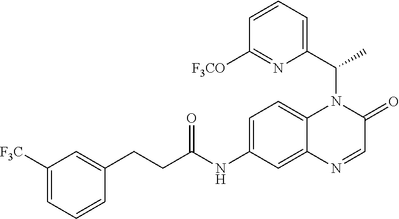 [SMILES: C[C@@H](c1cccc(OC(F)(F)F)n1)n1c(=O)cnc2cc(NC(=O)CCc3cccc(C(F)(F)F)c3)ccc21]

### (S)—N-(2-oxo-1-(1-(6-(trifluoromethoxy)pyridin-2-yl)ethyl)-1,2-dihydroquinoxalin-6-yl)-3-(3-(trifluoromethyl)phenyl)propanamide

To a solution of 6-amino-1-[(1S)-1-[6-(trifluoromethoxy)pyridin-2-yl]ethyl]quinoxalin-2-one (90 mg, 0.257 mmol, 1 equiv) in DMF (10 mL) was added 3-[3-(trifluoromethyl)phenyl]propanoic acid (61.66 mg, 0.283 mmol, 1.1 equiv), HATU (127.00 mg, 0.334 mmol, 1.3 equiv) and DIEA (99.62 mg, 0.771 mmol, 3 equiv) at 0° C. Quenched with water (30 mL) and extracted with EA (3×30 mL). The combined extracts were washed with water, brine, dried over Na₂SO₄, filtered and concentrated. The residue was purified by preparative HPLC using a XBridge Prep OBD C18 Column, 30*150 mm, 5 m column (eluent: 43% to 73% (v/v) CH₃CN and H₂O with 10 mmol/L NH₄HCO₃) to afford the title compound N-{2-oxo-1-[(1S)-1-[6-(trifluoromethoxy)pyridin-2-yl]ethyl]quinoxalin-6-yl}-3-[3-(trifluoromethyl)phenyl]propanamide (62.6 mg, 44.26%) as a yellow solid. LC/MS (ESI): mass calcd. for C₂₆H₂₀F₆N₄O₃:550.14 m/z, found: 551.15 [M+H]⁺. ¹H NMR (400 MHz, DMSO-d₆) δ 10.14 (s, 1H), 8.24 (s, 1H), 8.14 (d, J=2.5 Hz, 1H), 7.85-8.09 (m, 1H), 7.46-7.80 (m, 6H), 7.39-7.46 (m, 1H), 7.09-7.20 (m, 1H), 6.22-6.74 (m, 1H), 2.94-3.11 (m, 2H), 2.60-2.78 (m, 2H), 1.89 (d, J=7.0 Hz, 3H). ¹⁹F NMR (376 MHz, DMSO) δ(ppm): −55.3657, −60.9601, −61.0139.

### Example 319: (S)-1-(1-(1-(3-chlorophenyl)ethyl)-2-oxo-1,2-dihydroquinoxalin-6-yl)-3-(1-methylcyclobutyl)urea

### Synthetic Scheme

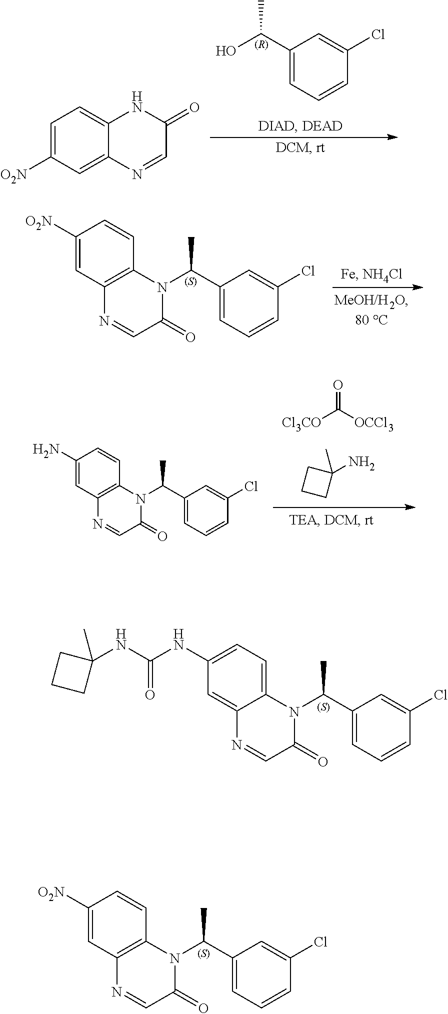

### (S)-1-(1-(3-chlorophenyl)ethyl)-6-nitroquinoxalin-2(1H)-one

To a solution of 6-nitro-1H-quinoxalin-2-one (852 mg, 4.457 mmol, 1 equiv) in DCM (10 mL) was added (1R)-1-(3-chlorophenyl)ethanol (698.06 mg, 4.457 mmol, 1 equiv) and PPh₃ (1753.68 mg, 6.685 mmol, 1.5 equiv). The reaction was stirred at rt under N₂ for 0.5 h. To the above mixture was added DEAD (1164.41 mg, 6.685 mmol, 1.5 equiv) at 0° C. The resulting mixture was stirred for additional 1 h at rt. Quenched with water (50 mL) and extracted with DCM (3×50 mL). The combined extracts were washed with water, brine, dried over Na₂SO₄, filtered and concentrated. The residue was purified by column chromatography on silica gel with EA/PE (0-100%) to give 1-[(1S)-1-(3-chlorophenyl)ethyl]-6-nitroquinoxalin-2-one (200 mg, 13.47%) as a yellow oil. MS (ESI): mass calcd. for C₁₆H₁₂ClN₃O₃: 329.06 m/z, found 329.95 [M+H]⁺.

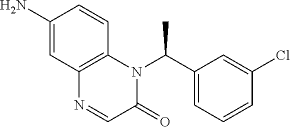 [SMILES: C[C@@H](c1cccc(Cl)c1)n1c(=O)cnc2cc(N)ccc21]

### (S)-6-amino-1-(1-(3-chlorophenyl)ethyl)quinoxalin-2(1H)-one

To a solution of 1-[(1S)-1-(3-chlorophenyl)ethyl]-6-nitroquinoxalin-2-one (190 mg, 0.576 mmol, 1 equiv) in MeOH/H₂O (10:1, 11 mL) was added Fe (321.79 mg, 5.760 mmol, 10 equiv) and NH₄C₁ (308.22 mg, 5.760 mmol, 10 equiv). The reaction was stirred at 80° C. for 1 h. After cooling down to rt, the resulting mixture was filtered, the filter cake was washed with MeOH (3×20 mL). The filtrate was concentrated under reduced pressure. Diluted with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 6-amino-1-[(1S)-1-(3-chlorophenyl)ethyl]quinoxalin-2-one (100 mg, 57.90%) as a yellow solid. MS (ESI): mass calcd. for C₁₆H₁₄ClN₃O: 299.08 m/z, found 300.00 [M+H]⁺.

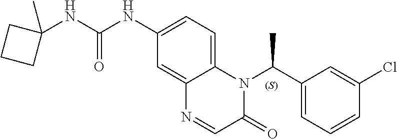

### (S)-1-(1-(1-(3-chlorophenyl)ethyl)-2-oxo-1,2-dihydroquinoxalin-6-yl)-3-(1-methylcyclobutyl)urea

To a solution of 6-amino-1-[(1S)-1-(3-chlorophenyl)ethyl]quinoxalin-2-one (90 mg, 0.300 mmol, 1 equiv) in DCM (10 mL) was added triphosgene (35.64 mg, 0.120 mmol, 0.4 equiv) under N₂ at 0° C. The reaction was stirred at 0° C. under N₂ for 1 h. Then, to the above mixture was added TEA (91.15 mg, 0.900 mmol, 3 equiv) under N₂ at 0° C. The resulting mixture was stirred for additional 1 h at rt. Then, 1-methylcyclobutan-1-amine (51.13 mg, 0.600 mmol, 2 equiv) was added. The reaction was stirred at rt for 1 h. Quenched with water (20 mL) and extracted with DCM (3×20 mL). The combined extracts were washed with water, brine, dried over Na₂SO₄, filtered and concentrated. The residue was purified by preparative HPLC using a XBridge Shield RP 18 OBD Column, 30*150 mm, 5 m column (eluent: 36% to 66% (v/v) CH₃CN and Water (10 mmol/L NH₄HCO₃+0.1% NH₃·H₂O)) to afford the title compound 1-{1-[(1S)-1-(3-chlorophenyl)ethyl]-2-oxoquinoxalin-6-yl}-3-(1-methylcyclobutyl)urea (27.7 mg, 22.45%) as a yellow solid. LC/MS (ESI): mass calcd. for C₂₂H₂₃ClN₄O₂:410.15 m/z, found: 411.15 [M+H]⁺. ¹H NMR (400 MHz, DMSO-d₆) δ 8.44 (s, 1H), 8.25 (s, 1H), 7.96 (d, J=2.5 Hz, 1H), 7.27-7.41 (m, 4H), 7.21 (d, J=6.9 Hz, 1H), 6.93-7.16 (m, 1H), 6.55 (s, 1H), 6.33 (s, 1H), 2.18-2.33 (m, 2H), 1.86-1.94 (m, 5H), 1.70-1.82 (m, 2H), 1.39 (s, 3H).

### Example 320: (S)-1-(1-methylcyclobutyl)-3-(2-oxo-1-(1-(3-(trifluoromethyl)phenyl)ethyl)-1,2-dihydroquinoxalin-6-yl)urea

### Synthetic Scheme

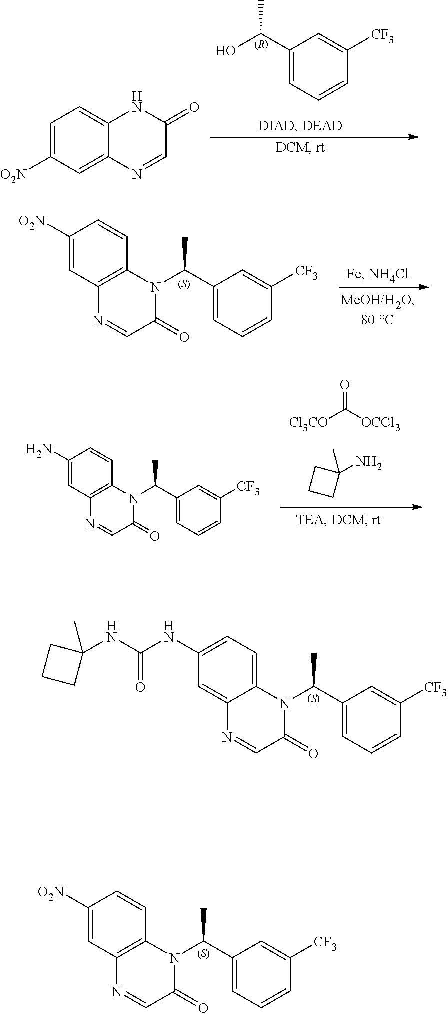

### (S)-6-nitro-1-(1-(3-(trifluoromethyl)phenyl)ethyl)quinoxalin-2(1H)-one

To a solution of 6-nitro-1H-quinoxalin-2-one (704 mg, 3.683 mmol, 1 equiv) in DCM (10 mL) was added (1R)-1-[3-(trifluoromethyl)phenyl]ethanol (700.39 mg, 3.683 mmol, 1 equiv) and PPh₃ (1449.05 mg, 5.524 mmol, 1.5 equiv). The reaction was stirred at rt under N₂ for 0.5 h. To the above mixture was added DEAD (962.14 mg, 5.524 mmol, 1.5 equiv) at 0° C. The resulting mixture was stirred for additional 1 h at rt. Quenched with water (50 mL) and extracted with DCM (3×50 mL). The combined extracts were washed with water, brine, dried over Na₂SO₄, filtered and concentrated. The residue was purified by column chromatography on silica gel with EA/PE (0-100%) to give 6-nitro-1-[(1S)-1-[3-(trifluoromethyl)phenyl]ethyl]quinoxalin-2-one (200 mg, 14.35%) as a yellow oil. MS (ESI): mass calcd. for C₁₇H₁₂F₃N₃O₃: 363.08 m/z, found 364.00 [M+H]⁺.

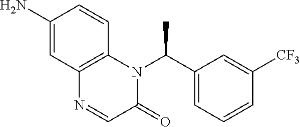 [SMILES: C[C@@H](c1cccc(C(F)(F)F)c1)n1c(=O)cnc2cc(N)ccc21]

### (S)-6-amino-1-(1-(3-(trifluoromethyl)phenyl)ethyl)quinoxalin-2(1H)-one

To a solution of 6-nitro-1-[(1S)-1-[3-(trifluoromethyl)phenyl]ethyl]quinoxalin-2-one (190 mg, 0.523 mmol, 1 equiv) in MeOH/H₂O (10:1, 11 mL) was added Fe (292.06 mg, 5.230 mmol, 10 equiv) and NH₄C₁ (279.75 mg, 5.230 mmol, 10 equiv). The reaction was stirred at 80° C. for 1 h. After cooling down to rt, the resulting mixture was filtered, the filter cake was washed with MeOH (3×20 mL). The filtrate was concentrated under reduced pressure. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 6-amino-1-[(1S)-1-[3-(trifluoromethyl)phenyl]ethyl]quinoxalin-2-one (96 mg, 55.07%) as a yellow solid. MS (ESI): mass calcd. for C₁₇H₁₄F₃N₃O: 333.11 m/z, found 334.00 [M+H]⁺.

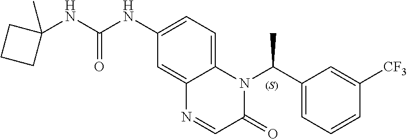

### (S)-1-(1-methylcyclobutyl)-3-(2-oxo-1-(1-(3-(trifluoromethyl)phenyl)ethyl)-1,2-dihydroquinoxalin-6-yl)urea

To a solution of 6-amino-1-[(1S)-1-[3-(trifluoromethyl)phenyl]ethyl]quinoxalin-2-one (80 mg, 0.240 mmol, 1 equiv) in DCM (10 mL) was added triphosgene (28.49 mg, 0.096 mmol, 0.4 equiv) under N₂ at 0° C. The reaction was stirred at 0° C. under N₂ for 1 h. Then, to the above mixture was added TEA (72.86 mg, 0.720 mmol, 3 equiv) under N₂ at 0° C. The resulting mixture was stirred for additional 1 h at rt. Then, to the above mixture was added 1-methylcyclobutan-1-amine (40.87 mg, 0.480 mmol, 2 equiv). The reaction was stirred at rt for 1 h. Quenched with water (20 mL) and extracted with DCM (3×20 mL). The combined extracts were washed with water, brine, dried over Na₂SO₄, filtered and concentrated. The residue was purified by preparative HPLC using a XBridge Shield RP 18 OBD Column, 30*150 mm, 5 μm column (eluent: 36% to 66% (v/v) CH₃CN and Water (10 mmol/L NH₄HCO₃+0.1% NH₃·H₂O) to afford the title compound 3-(1-methylcyclobutyl)-1-{2-oxo-1-[(1S)-1-[3-(trifluoromethyl)phenyl]ethyl]quinoxalin-6-yl}urea (22.2 mg, 20.81%) as a yellow solid. MS (ESI): mass calcd. for C₂₃H₂₃F₃N₄O₂: 444.18 m/z, found: 445.20 [M+H]⁺. ¹H NMR (400 MHz, DMSO-d₆) δ 8.45 (s, 1H), 8.24 (s, 1H), 7.96 (d, J=2.5 Hz, 1H), 7.50-7.70 (m, 4H), 7.35 (s, 1H), 6.90-7.27 (m, 1H), 6.40-6.78 (m, 1H), 6.33 (s, 1H), 2.21-2.33 (m, 2H), 1.85-1.97 (m, 5H), 1.71-1.80 (m, 2H), 1.88-1.72 (m, 2H), 1.39 (s, 3H). ¹⁹F NMR (376 MHz, DMSO) δ(ppm): −60.8769.

### Example 321: (R)-1-(2-methyl-3-oxo-4-((6-(trifluoromethoxy)pyridin-2-yl)methyl)-3,4-dihydro-2H-benzo[b][1,4]oxazin-7-yl)-3-(1-methylcyclopentyl)urea

### Synthetic Scheme

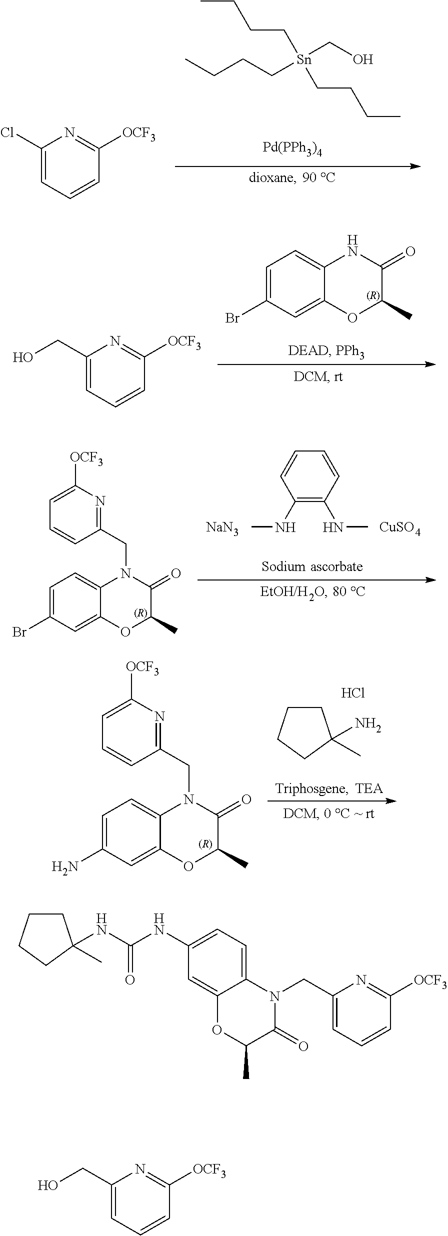

### (6-(trifluoromethoxy)pyridin-2-yl)methanol

To a solution of 2-chloro-6-(trifluoromethoxy)pyridine (800 mg, 4.050 mmol, 1 equiv) in dioxane (8.0 mL) was added (tripropylstannyl)methanol (1694.91 mg, 6.075 mmol, 1.5 equiv) and Pd(PPh₃)₄ (467.99 mg, 0.405 mmol, 0.1 equiv). The reaction was stirred at 90° C. under N₂ for 12 h. The mixture was allowed to cool down to rt. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give [6-(trifluoromethoxy)pyridin-2-yl]methanol (500 mg, 63.93%) as a white solid. MS (ESI): mass calcd. for C₇H₆F₃NO₂: 193.04 m/z, found 194.05 [M+H]⁺.

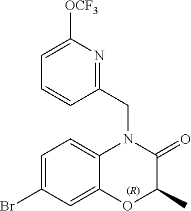

### (R)-7-bromo-2-methyl-4-((6-(trifluoromethoxy)pyridin-2-yl)methyl)-2H-benzo[b][1,4]oxazin-3(4H)-one

A solution of (2R)-7-bromo-2-methyl-2,4-dihydro-1,4-benzoxazin-3-one (451.24 mg, 1.864 mmol, 1.2 equiv) in DCM (5.0 mL) was treated with [6-(trifluoromethoxy)pyridin-2-yl]methanol (300 mg, 1.553 mmol, 1 equiv) and PPh₃ (611.17 mg, 2.329 mmol, 1.5 equiv) for 1 h at rt under nitrogen atmosphere followed by the addition of DEAD (270.53 mg, 1.553 mmol, 1 equiv) dropwise at 0° C. The resulting mixture was stirred for 2 h at rt under N₂ atmosphere. Quenched with water (10 mL) and extracted with DCM (3×10 mL). The combined extracts were washed with water, brine, dried over Na₂SO₄, filtered and concentrated. The residue was purified by column chromatography on silica gel with EA/PE (0-100%) to give (2R)-7-bromo-6-fluoro-2-methyl-4-[(1S)-1-phenylethyl]-2H-1,4-benzoxazin-3-one (195 mg, 46.41%) as a white solid. MS (ESI): mass calcd. for C₁₆H₁₂BrF₃N₂O₃: 416.00 m/z, found 416.95, 418.95 [M+H, M+H+2]⁺.

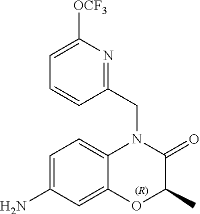

### (R)-7-amino-2-methyl-4-((6-(trifluoromethoxy)pyridin-2-yl)methyl)-2H-benzo[b][1,4]oxazin-3(4H)-one

To a solution of (2R)-7-bromo-2-methyl-4-{[6-(trifluoromethoxy)pyridin-2-yl]methyl}-2H-1,4-benzoxazin-3-one (180 mg, 0.373 mmol, 1 equiv) in EtOH (3 mL)/H₂O (0.3 mL) was added N1,N2-dimethylcyclohexane-1,2-diamine (31.86 mg, 0.224 mmol, 0.6 equiv), CuSO₄(59.58 mg, 0.373 mmol, 1 equiv), sodium ascorbate (148.66 mg, 0.746 mmol, 2 equiv) and NaN₃ (72.80 mg, 1.119 mmol, 3 equiv). The reaction was stirred at 90° C. under N₂ for 2 h. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give (2R)-7-amino-2-methyl-4-{[6-(trifluoromethoxy)pyridin-2-yl]methyl}-2H-1,4-benzoxazin-3-one (91 mg, 69.00%) as a white solid. LC/MS (ESI): mass calcd. for C₁₆H₁₄F₃N₃O₃: 353.10 m/z, found: 354.00 [M+H]⁺.

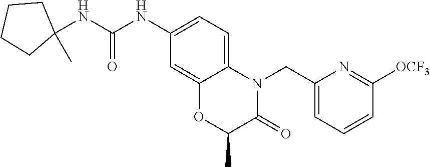 [SMILES: C[C@H]1Oc2cc(NC(=O)NC3(C)CCCC3)ccc2N(Cc2cccc(OC(F)(F)F)n2)C1=O]

### (R)-1-(2-methyl-3-oxo-4-((6-(trifluoromethoxy)pyridin-2-yl)methyl)-3,4-dihydro-2H-benzo[b][1,4]oxazin-7-yl)-3-(1-methylcyclopentyl)urea

To a solution of (2R)-7-amino-2-methyl-4-{[6-(trifluoromethoxy)pyridin-2-yl]methyl}-2H-1,4-benzoxazin-3-one (85 mg, 0.241 mmol, 1 equiv) in DCM (3 mL) was added Triphosgene (71.39 mg, 0.241 mmol, 1 equiv). The reaction was stirred at 0° C. under N₂ for 1 h. The reaction was added TEA (194.77 mg, 1.928 mmol, 8 equiv) and stirred at 0° C. under N₂ for 1 h. The reaction was added 1-methyl-cyclopentanamine (95.44 mg, 0.964 mmol, 4 equiv) and stirred at rt under N₂ for 1 h. Quenched with water (10 mL) and extracted with DCM (3×10 mL). The combined extracts were washed with water, brine, dried over Na₂SO₄, filtered and concentrated. The residue was purified by preparative HPLC using a YMC-Actus Triart C18 ExRS 150 mm×30 mm×5 μm column (eluent: 49% to 73% (v/v) CH₃CN and H₂O with 10 mmol/L NH₄HCO₃) to afford the title compound 1-[(2R)-2-methyl-3-oxo-4-{[6-(trifluoromethoxy)pyridin-2-yl]methyl}-2H-1,4-benzoxazin-7-yl]-3-(1-methylcyclopentyl)urea (35.8 mg, 31.05%) as a white solid. LC/MS (ESI): mass calcd. for C₂₃H₂₅F₃N₄O₄:478.18 m/z, found: 479.05 [M+H]⁺. ¹H NMR (300 MHz, DMSO-d₆) δ 8.21 (s, 1H), 7.96-7.99 (m, 1H), 7.29-7.32 (m, 1H), 7.22-7.23 (m, 1H), 7.15-7.17 (m, 1H), 6.82-6.85 (m, 1H), 6.74-7.78 (m, 1H), 6.02 (s, 1H), 5.14 (s, 2H), 4.73-7.79 (m, 1H), 1.87-1.89 (m, 2H), 1.56-1.62 (m, 4H), 1.44-1.54 (m, 5H), 1.34 (s, 3H). ¹⁹F NMR (282 MHz, DMSO) δ−55.06.

### Example 322: 1-((R)-2-methyl-3-oxo-4-((S)-1-(6-(trifluoromethyl)pyridin-2-yl)ethyl)-3,4-dihydro-2H-benzo[b][1,4]oxazin-7-yl)-3-(1-methylcyclopentyl)urea

### Synthetic Scheme

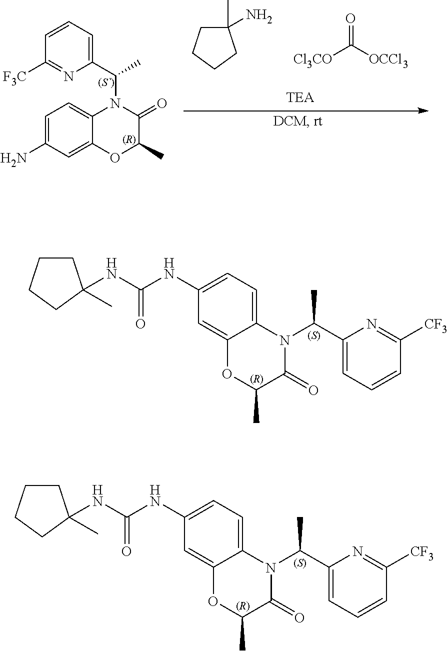

### 1-((R)-2-methyl-3-oxo-4-((S)-1-(6-(trifluoromethyl)pyridin-2-yl)ethyl)-3,4-dihydro-2H-benzo[b][1,4]oxazin-7-yl)-3-(1-methylcyclopentyl)urea

To a solution of (2R)-7-amino-2-methyl-4-[(1S)-1-[6-(trifluoromethyl)pyridin-2-yl]ethyl]-2H-1,4-benzoxazin-3-one (70 mg, 0.199 mmol, 1 equiv) in DCM (5 mL) was added triphosgene (23.65 mg, 0.080 mmol, 0.4 equiv) under N₂ at 0° C. The reaction was stirred at 0° C. under N₂ for 1 h. Then, to the above mixture was added TEA (60.49 mg, 0.597 mmol, 3 equiv) under N₂ at 0° C. The resulting mixture was stirred for additional 1 h at rt. Then, 1-methyl-cyclopentanamine (39.52 mg, 0.398 mmol, 2 equiv) was added. The reaction was stirred at rt for 1 h. Quenched with water (10 mL) and extracted with DCM (3×10 mL). The combined extracts were washed with water, brine, dried over Na₂SO₄, filtered and concentrated. The residue was purified by preparative HPLC using a XBridge Shield RP 18 OBD Column, 30*150 mm, 5 μm column (eluent: 48% to 78% (v/v) CH₃CN and H₂O (10 mmol/L NH₄HCO₃+0.1% NH₃·H₂O)) to afford the title compound 1-[(2R)-2-methyl-3-oxo-4-[(1S)-1-[6-(trifluoromethyl)pyridin-2-yl]ethyl]-2H-1,4-benzoxazin-7-yl]-3-(1-methylcyclopentyl)urea (16.7 mg, 17.59%) as a yellow solid. LC/MS (ESI): mass calcd. for C₂₄H₂₇F₃N₄O₃:476.20 m/z, found: 477.05 [M+H]⁺. ¹H NMR (300 MHz, DMSO-d₆) δ 8.19 (s, 1H), 7.97-8.11 (m, 1H), 7.70-7.90 (m, 1H), 7.52-7.67 (m, 1H), 7.23 (d, J=1.9 Hz, 1H), 6.63-6.80 (m, 2H), 5.95-6.15 (m, 2H), 4.63-4.80 (m, 1H), 1.84-1.99 (m, 2H), 1.74-1.84 (m, 3H), 1.57-1.69 (m, 4H), 1.47-1.54 (m, 2H), 1.37-1.42 (m, 3H), 1.33 (s, 3H). ¹⁹F NMR (282 MHz, DMSO) δ(ppm): −66.560.

### Example 323: 1-((R)-2-methyl-3-oxo-4-((S)-1-(6-(trifluoromethoxy)pyridin-2-yl)ethyl)-3,4-dihydro-2H-benzo[b][1,4]oxazin-7-yl)-3-(1-methylcyclopentyl)urea

### Synthetic Scheme

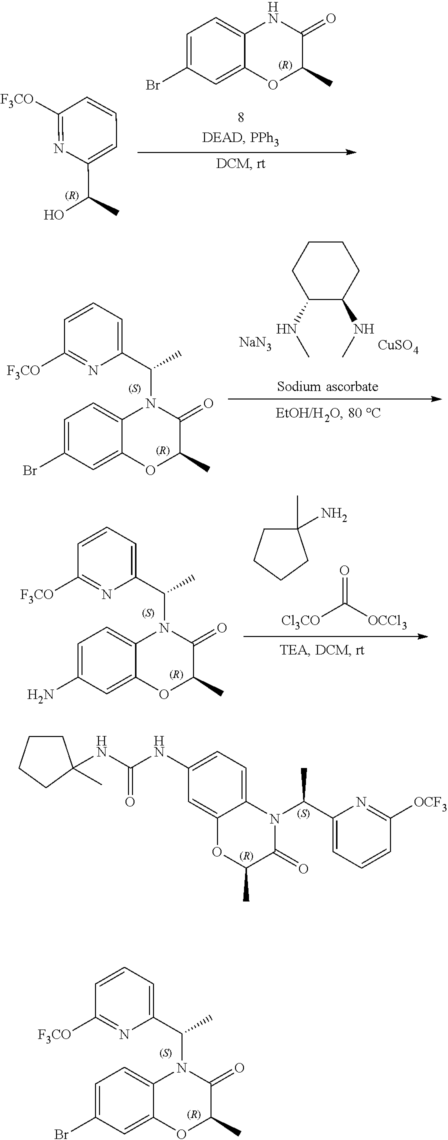

### (R)-7-bromo-2-methyl-4-((S)-1-(6-(trifluoromethoxy)pyridin-2-yl)ethyl)-2H-benzo[b][1,4]oxazin-3 (4H)-one

To a solution of (1R)-1-[6-(trifluoromethoxy)pyridin-2-yl]ethanol (300 mg, 1.448 mmol, 1 equiv) in DCM (10 mL) was added (2R)-7-bromo-2-methyl-2,4-dihydro-1,4-benzoxazin-3-one (350.57 mg, 1.448 mmol, 1 equiv) and PPh₃ (569.78 mg, 2.172 mmol, 1.5 equiv). The reaction was stirred at rt under N₂ for 0.5 h. To the above mixture was added DEAD (378.32 mg, 2.172 mmol, 1.5 equiv) at 0° C. The resulting mixture was stirred for additional 1 h at rt. Quenched with water (20 mL) and extracted with DCM (3×20 mL). The combined extracts were washed with water, brine, dried over Na₂SO₄, filtered and concentrated. The residue was purified by column chromatography on silica gel with EA/PE (0-100%) to give (2R)-7-bromo-2-methyl-4-[(1S)-1-[6-(trifluoromethoxy)pyridin-2-yl]ethyl]-2H-1,4-benzoxazin-3-one (220 mg, 35.23%) as a yellow solid. LC/MS: mass calcd, for C₁₇H₁₄BrF₃N₂O₃:430.01, found: 430.95, 432.95 [M+H, M+H+2]⁺.

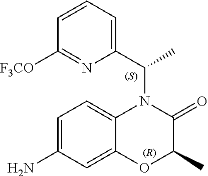

### (R)-7-amino-2-methyl-4-((S)-1-(6-(trifluoromethoxy)pyridin-2-yl)ethyl)-2H-benzo[b][1,4]oxazin-3 (4H)-one

To a solution of (2R)-7-bromo-2-methyl-4-[(1S)-1-[6-(trifluoromethoxy)pyridin-2-yl]ethyl]-2H-1,4-benzoxazin-3-one (210 mg, 0.487 mmol, 1 equiv) in EtOH/H₂O (4:1, 10 mL) was added (1R,2R)—N1,N2-dimethylcyclohexane-1,2-diamine (41.56 mg, 0.292 mmol, 0.6 equiv), CuSO₄ (77.73 mg, 0.487 mmol, 1 equiv), azidosodium (94.98 mg, 1.461 mmol, 3 equiv) and sodium ascorbate (193.94 mg, 0.974 mmol, 2 equiv). The reaction was stirred at 80° C. under N₂ for 3 h. After cooling down to rt, the reaction was quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-7-amino-2-methyl-4-[(1S)-1-[6-(trifluoromethoxy)pyridin-2-yl]ethyl]-2H-1,4-benzoxazin-3-one (130 mg, 72.67%) as a yellow solid. MS (ESI): mass calcd. for C₁₇H₁₆F₃N₃O₃: 367.11 m/z, found 368.05 [M+H]⁺.

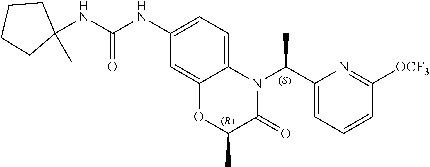

### 1-((R)-2-methyl-3-oxo-4-((S)-1-(6-(trifluoromethoxy)pyridin-2-yl)ethyl)-3,4-dihydro-2H-benzo[b][1,4]oxazin-7-yl)-3-(1-methylcyclopentyl)urea

To a solution of (2R)-7-amino-2-methyl-4-[(1S)-1-[6-(trifluoromethoxy)pyridin-2-yl]ethyl]-2H-1,4-benzoxazin-3-one (60 mg, 0.163 mmol, 1 equiv) in DCM (5 mL) was added triphosgene (19.39 mg, 0.065 mmol, 0.4 equiv) under N₂ at 0° C. The reaction was stirred at 0° C. under N₂ for 1 h. To the above mixture was added TEA (49.59 mg, 0.489 mmol, 3 equiv) under N₂ at 0° C. The resulting mixture was stirred for additional 1 h at rt. Then, 1-methyl-cyclopentanamine (32.40 mg, 0.326 mmol, 2 equiv) was added. The reaction was stirred at rt for 1 h. Quenched with water (10 mL) and extracted with DCM (3×10 mL). The combined extracts were washed with water, brine, dried over Na₂SO₄, filtered and concentrated. The residue was purified by preparative HPLC using a XBridge Prep OBD C18 Column30*150 mmm column (eluent: 30% to 55% (v/v) CH₃CN and Water (10 mmol/L NH₄HCO₃)) to afford the title compound 1-[(2R)-2-methyl-3-oxo-4-[(1S)-1-[6-(trifluoromethoxy)pyridin-2-yl]ethyl]-2H-1,4-benzoxazin-7-yl]-3-(1-methylcyclopentyl)urea (23.1 mg, 28.72%) as a white solid. LC/MS (ESI): mass calcd. for C₂₄H₂₇F₃N₄O₄: 492.20 m/z, found: 493.10 [M+H]⁺. ¹H NMR (300 MHz, DMSO-d₆) δ 8.19 (s, 1H), 8.00 (t, J=7.9 Hz, 1H), 7.35 (d, J=7.7 Hz, 1H), 7.10-7.20 (m, 2H), 6.60-6.78 (m, 2H), 5.90-6.11 (m, 2H), 4.55-4.80 (m, 1H), 1.81-2.00 (m, 2H), 1.75 (d, J=7.1 Hz, 3H), 1.45-1.70 (m, 6H), 1.38-1.45 (s, 3H), 1.25-1.38 (m, 3H). ¹⁹F NMR (282 MHz, DMSO) δ (ppm): −55.093

### Example 324: 1-((R)-2-methyl-3-oxo-4-((S)-1-(3-(trifluoromethoxy)phenyl)ethyl)-3,4-dihydro-2H-benzo[b][1,4]oxazin-7-yl)-3-((1r,3R)-3-(trifluoromethyl)cyclobutyl)urea

### Synthetic Scheme

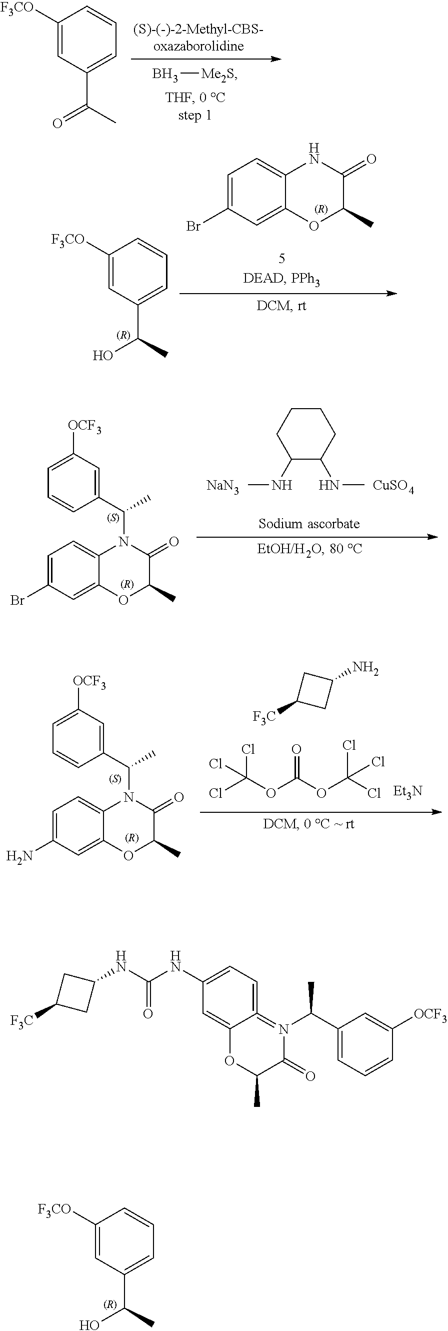

### (R)-1-(3-(trifluoromethoxy)phenyl)ethan-1-ol

To a solution of BH₃-Me₂S (279 mg, 3.676 mmol, 1.5 equiv) and (S)-(−)-2-Methyl-CBS-oxazaborolidine (135 mg, 0.490 mmol, 0.2 equiv) in THF (6 mL) was added 1-(3-(trifluoromethoxy)phenyl)ethan-1-one (500 mg, 2.450 mmol, 1 equiv) at 0° C. The resulting mixture was stirred for 2 h at 0° C. The reaction mixture was quenched by water (10 mL) and extracted with EA (3×30 mL). The combined organic extracts were washed with brine (30 mL), dried over anhydrous Na₂SO₄ and concentrated under vacuum. The crude product was purified by flash chromatography with PE/EA (3:1) to afford (R)-1-(3-(trifluoromethoxy)phenyl)ethan-1-ol (0.4 g, 80.00%) as a yellow oil.

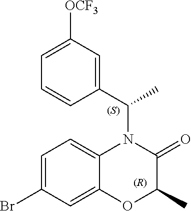

### (R)-7-bromo-2-methyl-4-((S)-1-(3-(trifluoromethoxy)phenyl)ethyl)-2H-benzo[b][1,4]oxazin-3(4H)-one

To a solution of (R)-1-(3-(trifluoromethoxy)phenyl)ethan-1-ol (0.4 g, 1.942 mmol, 1 equiv) in DCM (6 mL) was added (R)-7-bromo-2-methyl-2H-benzo[b][1,4]oxazin-3(4H)-one (469 mg, 1.942 mmol, 1 equiv), triphenylphosphine (510 mg, 1.942 mmol, 1 equiv) and DEAD (338 mg, 1.942 mmol, 1 equiv) at 0° C. under a nitrogen atmosphere. The resulting mixture was stirred overnight at rt. The reaction mixture was quenched by water (10 mL) and extracted with EA (3×30 mL). The combined organic extracts were washed with brine (30 mL), dried over anhydrous Na₂SO₄ and concentrated under vacuum. The crude product was purified by flash chromatography with PE/EA (3:1) to afford (R)-7-bromo-2-methyl-4-((S)-1-(3-(trifluoromethoxy)phenyl)ethyl)-2H-benzo[b][1,4]oxazin-3(4H)-one (0.3 g, 36.06%) as a yellow solid.

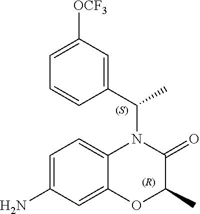

### (R)-7-amino-2-methyl-4-((S)-1-(3-(trifluoromethoxy)phenyl)ethyl)-2H-benzo[b][1,4]oxazin-3(4H)-one

To a solution of (R)-7-bromo-2-methyl-4-((S)-1-(3-(trifluoromethoxy)phenyl)ethyl)-2H-benzo[b][1,4]oxazin-3(4H)-one (0.3 g, 0.699 mmol, 1 equiv) in EtOH (6 mL) & H₂O (2 mL) was added sodium ascorbate (28 mg, 0.140 mmol, 0.2 equiv), N¹,N²-dimethylcyclohexane-1,2-diamine (20 mg, 0.140 mmol, 0.2 equiv), CuSO₄ (23 mg, 0.14 mmol, 0.2 equiv) and NaN₃ (136 mg, 2.097 mmol, 3 equiv) at rt. The resulting mixture was stirred 4 h at 80° C. The reaction mixture was quenched by water (10 mL) and extracted with EA (3×30 mL). The combined organic extracts were washed with brine (30 mL), dried over anhydrous Na₂SO₄ and concentrated under vacuum. The crude product was purified by flash chromatography with PE/EA (1:1) to afford (R)-7-amino-2-methyl-4-((S)-1-(3-(trifluoromethoxy)phenyl)ethyl)-2H-benzo[b][1,4]oxazin-3(4H)-one (166 mg, 65.02%) as a yellow solid.

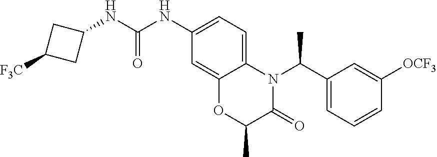 [SMILES: C[C@H]1Oc2cc(NC(=O)N[C@H]3C[C@H](C(F)(F)F)C3)ccc2N([C@@H](C)c2cccc(OC(F)(F)F)c2)C1=O]

### 1-((R)-2-methyl-3-oxo-4-((S)-1-(3-(trifluoromethoxy)phenyl)ethyl)-3,4-dihydro-2H-benzo[b][1,4]oxazin-7-yl)-3-((1r,3R)-3-(trifluoromethyl)cyclobutyl)urea

To a mixture of (R)-7-amino-2-methyl-4-((S)-1-(3-(trifluoromethoxy)phenyl)ethyl)-2H-benzo[b][1,4]oxazin-3(4H)-one (60 mg, 0.163 mmol, 1 equiv) in DCM (4 mL) was stirred under nitrogen atmosphere and added triphosgene (15 mg, 0.149 mmol, 0.3 equiv) at 0° C. The reaction was stirred at rt for 1 h. TEA (41 mg, 0.407 mmol, 2.5 equiv) was added into the mixture at 0° C. The reaction was stirred at rt for 1 h. (1r,3r)-3-(trifluoromethyl)cyclobutan-1-amine (23 mg, 0.163 mmol, 1.0 equiv) was added into the mixture at 0° C. The reaction was stirred at rt for 1 h. The reaction mixture was quenched by 1N hydrochloric acid (5 mL) and extracted with DCM (3×10 mL). The organic layers were combined, dried over anhydrous sodium sulfate, filtered and concentrated under vacuum. The crude product was purified by silica gel column chromatography, eluted with PE/EA (1:1) to afford 1-((R)-2-methyl-3-oxo-4-((S)-1-(3-(trifluoromethoxy)phenyl)ethyl)-3,4-dihydro-2H-benzo[b][1,4]oxazin-7-yl)-3-((1r,3R)-3-(trifluoromethyl)cyclobutyl)urea (25.5 mg, 29.44%) as a white solid. MS (ESI): mass calcd. for C₂₄H₂₃F₆N₃O₄: 531.15 m/z, found: 532.15 [M+H]⁺. ¹H NMR (300 MHz, DMSO-d₆) δ 8.45 (s, 1H), 7.40-7.60 (m, 1H), 7.19-7.40 (m, 4H), 6.72-6.85 (m, 1H), 6.62-6.72 (m, 1H), 6.52-6.62 (m, 1H), 5.96-6.19 (m, 1H), 4.66-4.85 (m, 1H), 4.13-4.31 (m, 1H), 2.91-3.18 (m, 1H), 2.31-2.43 (m, 2H), 2.15-2.31 (m, 2H), 1.68-1.87 (m, 3H), 1.38-1.48 (m, 3H).

### Example 325: 1-((R)-2-methyl-3-oxo-4-((S)-1-(3-(trifluoromethoxy)phenyl)ethyl)-3,4-dihydro-2H-benzo[b][1,4]oxazin-7-yl)-3-((1s,3S)-3-(trifluoromethyl)cyclobutyl)urea

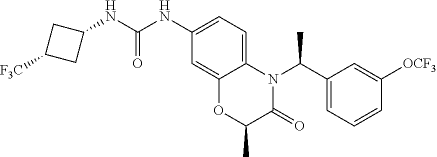 [SMILES: C[C@H]1Oc2cc(NC(=O)N[C@H]3C[C@@H](C(F)(F)F)C3)ccc2N([C@@H](C)c2cccc(OC(F)(F)F)c2)C1=O]

### 1-((R)-2-methyl-3-oxo-4-((S)-1-(3-(trifluoromethoxy)phenyl)ethyl)-3,4-dihydro-2H-benzo[b][1,4]oxazin-7-yl)-3-((1s,3S)-3-(trifluoromethyl)cyclobutyl)urea

To a mixture of (R)-7-amino-2-methyl-4-((S)-1-(3-(trifluoromethoxy)phenyl)ethyl)-2H-benzo[b][1,4]oxazin-3(4H)-one (60 mg, 0.163 mmol, 1 equiv) in DCM (4 mL) was stirred under nitrogen atmosphere and added triphosgene (15 mg, 0.149 mmol, 0.3 equiv) at 0° C. The reaction was stirred at rt for 1 h. TEA (41 mg, 0.407 mmol, 2.5 equiv) was added into the mixture at 0° C. The reaction was stirred at rt for 1 h. (1s,3s)-3-(trifluoromethyl)cyclobutan-1-amine (23 mg, 0.163 mmol, 1.0 equiv) was added into the mixture at 0° C. The reaction was stirred at rt for 1 h. The reaction mixture was quenched by 1N hydrochloric acid (5 mL) and extracted with DCM (3×10 mL). The organic layers were combined, dried over anhydrous sodium sulfate, filtered and concentrated under vacuum. The crude product was purified by silica gel column chromatography, eluted with PE/EA (1:1) to afford 1-((R)-2-methyl-3-oxo-4-((S)-1-(3-(trifluoromethoxy)phenyl)ethyl)-3,4-dihydro-2H-benzo[b][1,4]oxazin-7-yl)-3-((1s,3S)-3-(trifluoromethyl)cyclobutyl)urea (30.5 mg, 35.20%) as a white solid. MS (ESI): mass calcd. for C₂₄H₂₃F₆N₃O4: 531.15 m/z, found: 532.15 [M+H]⁺. ¹H NMR (300 MHz, DMSO-d₆) δ 8.32 (s, 1H), 7.40-7.60 (m, 1H), 7.19-7.22 (m, 4H), 6.72-6.85 (m, 1H), 6.62-6.72 (m, 1H), 6.52-6.62 (m, 1H), 5.96-6.19 (m, 1H), 4.66-4.85 (m, 1H), 4.02-4.22 (m, 1H), 2.79-2.98 (m, 1H), 2.35-2.49 (m, 2H), 1.85-2.05 (m, 2H), 1.72-1.85 (m, 3H), 1.38-1.46 (m, 3H).

### Example 326: (R)-1-(4-benzyl-6-fluoro-2-methyl-3-oxo-3,4-dihydro-2H-benzo[b][1,4]oxazin-7-yl)-3-(tert-butyl)urea

### Synthetic Scheme

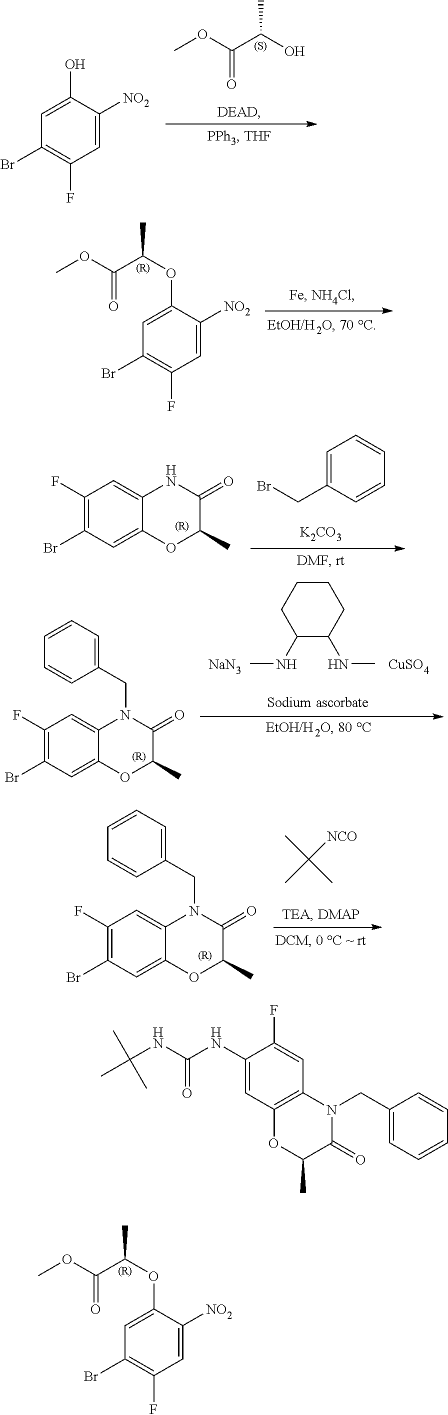

### methyl (R)-2-(5-bromo-4-fluoro-2-nitrophenoxy)propanoate

A solution of 5-bromo-4-fluoro-2-nitrophenol (1 g, 4.237 mmol, 1 equiv), methyl (2S)-2-hydroxypropanoate (0.66 g, 6.356 mmol, 1.5 equiv) and PPh₃ (1.67 g, 6.356 mmol, 1.5 equiv) in THF (10 mL) was stirred for 1 h at rt under N₂ atmosphere. To the above mixture was added DEAD (1.11 g, 6.356 mmol, 1.5 equiv) dropwise over 10 min at 0° C. The resulting mixture was stirred for additional 2 h at rt. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give methyl (2R)-2-(5-bromo-4-fluoro-2-nitrophenoxy)propanoate (1.21 g, 88.66%) as a white solid. MS (ESI): mass calcd. for C₁₀H₉BrFNO₅: 320.96 m/z, found 322.05, 324.05[M+H, M+H+2]⁺.

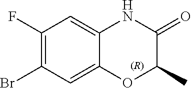

### (R)-7-bromo-6-fluoro-2-methyl-2H-benzo[b][1,4]oxazin-3(4H)-one

To a solution of methyl (2R)-2-(5-bromo-4-fluoro-2-nitrophenoxy)propanoate (1.2 g, 3.726 mmol, 1 equiv) in EtOH (12.0 mL)/H₂O (1.2 mL) was added Fe (2.1 g, 37.260 mmol, 10 equiv) and NH₄C₁ (2.0 g, 37.260 mmol, 10 equiv). The reaction was stirred at 70° C. for 3 h. The mixture was allowed to cool down to rt. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-7-bromo-6-fluoro-2-methyl-2,4-dihydro-1,4-benzoxazin-3-one (900 mg, 92.89%) as a white solid. MS (ESI): mass calcd. for C₉H₇BrFNO₂: 258.96 m/z, found 260.06, 262.06 [M+H, M+H+2]⁺.

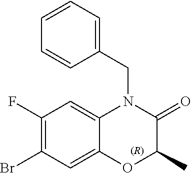

### (R)-4-benzyl-7-bromo-6-fluoro-2-methyl-2H-benzo[b][1,4]oxazin-3(4H)-one

To a solution of (2R)-7-bromo-6-fluoro-2-methyl-2,4-dihydro-1,4-benzoxazin-3-one (250 mg, 0.961 mmol, 1 equiv) in DMF (3 mL) was added benzyl bromide (493.26 mg, 2.883 mmol, 3 equiv) and K₂CO₃ (401.48 mg, 2.883 mmol, 3 equiv). The reaction was stirred at rt for 1 h. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give (2R)-4-benzyl-7-bromo-6-fluoro-2-methyl-2H-1,4-benzoxazin-3-one (250 mg, 74.26%) as a white solid. MS (ESI): mass calcd. for C₁₆H₁₃BrFNO₂: 349.01 m/z, found 349.95, 351.95 [M+H, M+H+2]⁺.

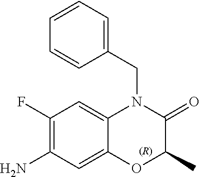

### (R)-7-amino-4-benzyl-6-fluoro-2-methyl-2H-benzo[b][1,4]oxazin-3(4H)-one

To a solution of (2R)-4-benzyl-7-bromo-6-fluoro-2-methyl-2H-1,4-benzoxazin-3-one (240 mg, 0.578 mmol, 1 equiv) in EtOH (5 mL)/H₂O (1 mL) was added N₁,N₂-dimethylcyclohexane-1,2-diamine (49.33 mg, 0.347 mmol, 0.6 equiv), CuSO₄(92.25 mg, 0.578 mmol, 1 equiv), sodium ascorbate (230.19 mg, 1.156 mmol, 2 equiv), and NaN₃ (112.74 mg, 1.734 mmol, 3 equiv). The reaction was stirred at 80° C. under N₂ for 2 h. The mixture was allowed to cool down to rt. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give (2R)-7-amino-4-benzyl-6-fluoro-2-methyl-2H-1,4-benzoxazin-3-one (150 mg, 90.64%) as a white solid. LC/MS (ESI): mass calcd. for C₁₆H₁₅FN₂O₂: 286.11 m/z, found: 287.20 [M+H]⁺.

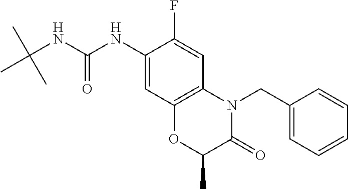 [SMILES: C[C@H]1Oc2cc(NC(=O)NC(C)(C)C)c(F)cc2N(Cc2ccccc2)C1=O]

### (R)-1-(4-benzyl-6-fluoro-2-methyl-3-oxo-3,4-dihydro-2H-benzo[b][1,4]oxazin-7-yl)-3-(tert-butyl)urea

To a solution of (2R)-7-amino-4-benzyl-6-fluoro-2-methyl-2H-1,4-benzoxazin-3-one (130 mg, 0.454 mmol, 1 equiv) and DMAP (5.55 mg, 0.045 mmol, 0.1 equiv) in DCM (3 mL) was added 2-isocyanato-2-methylpropane (135.04 mg, 1.362 mmol, 3 equiv) and TEA (137.84 mg, 1.362 mmol, 3 equiv). The reaction was stirred at rt for 24 h. Quenched with water (10 mL) and extracted with DCM (3×10 mL). The combined extracts were washed with water, brine, dried over Na₂SO₄, filtered and concentrated. The residue was purified by preparative HPLC using a YMC-Actus Triart C18 ExRS 150 mm×30 mm×5 μm column (eluent: 49% to 73% (v/v) CH₃CN and H₂O with 10 mmol/L NH₄HCO₃) to afford the title compound 1-[(2R)-4-benzyl-6-fluoro-2-methyl-3-oxo-2H-1,4-benzoxazin-7-yl]-3-tert-butylurea (34.9 mg, 19.74%) as a white solid. LC/MS (ESI): mass calcd. for C₂₁H₂₄FN₃O₃: 385.18 m/z, found: 408.10 [M+Na]⁺. ¹⁹F NMR (282 MHz, DMSO) δ −137.66. ¹H NMR (300 MHz, DMSO-d₆) δ 8.06 (s, 1H), 7.80-7.83 (d, J=7.7 Hz, 1H), 7.30-7.40 (m, 2H), 7.22-7.26 (m, 3H), 6.94-6.98 (d, J=12 Hz, 1H), 6.51 (s, 1H), 5.11 (s, 2H), 4.77-4.84 (m, 1H), 1.46-1.48 (d, J=6 Hz, 3H), 1.27 (s, 9H).

### Example 327: (R)-1-(tert-butyl)-3-(4-(3-(difluoromethyl)benzyl)-6-fluoro-2-methyl-3-oxo-3,4-dihydro-2H-benzo[b][1,4]oxazin-7-yl)urea

### Synthetic Scheme

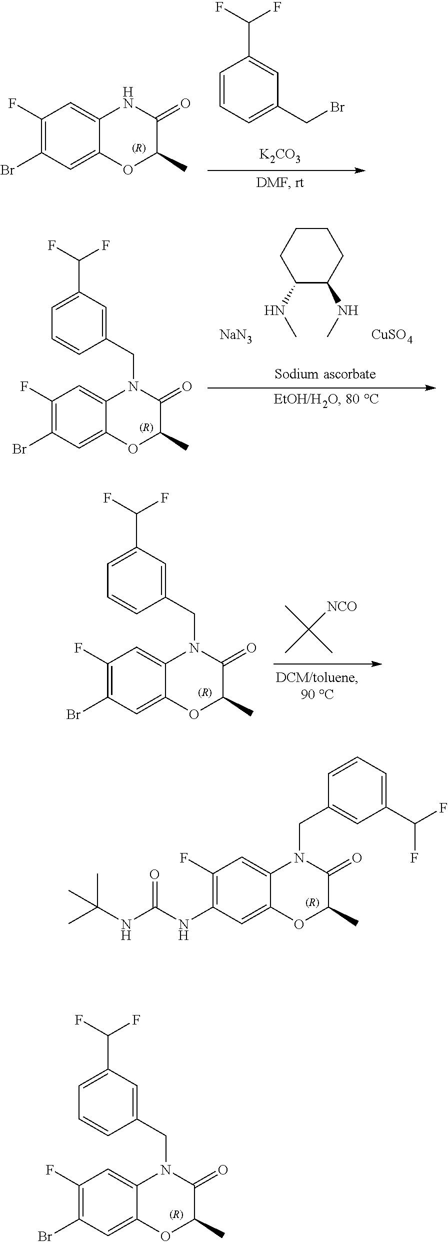

### (R)-7-bromo-4-(3-(difluoromethyl)benzyl)-6-fluoro-2-methyl-2H-benzo[b][1,4]oxazin-3(4H)-one

To a solution of (2R)-7-bromo-6-fluoro-2-methyl-2,4-dihydro-1,4-benzoxazin-3-one (250 mg, 0.961 mmol, 1 equiv) in DMF (10 mL) was added 1-(bromomethyl)-3-(difluoromethyl)benzene (637.48 mg, 2.883 mmol, 3 equiv) and K₂CO₃ (401.48 mg, 2.883 mmol, 3 equiv). The reaction was stirred at rt for 1 h. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-7-bromo-4-{[3-(difluoromethyl)phenyl]methyl}-6-fluoro-2-methyl-2H-1,4-benzoxazin-3-one (350 mg, 90.98%) as a yellow solid. LC/MS: mass calcd. for C₁₇H₁₃BrF₃NO₂:399.01 m/z, found: 399.90, 401.90 [M+H, M+H+2]⁺.

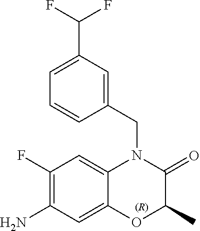

### (R)-7-amino-4-(3-(difluoromethyl)benzyl)-6-fluoro-2-methyl-2H-benzo[b][1,4]oxazin-3(4H)-one

To a solution of (2R)-7-bromo-4-{[3-(difluoromethyl)phenyl]methyl}-6-fluoro-2-methyl-2H-1,4-benzoxazin-3-one (340 mg, 0.850 mmol, 1 equiv) in EtOH (8 mL) and H₂O (2 mL) was added (1R,2R)—N₁,N₂-dimethylcyclohexane-1,2-diamine (72.51 mg, 0.510 mmol, 0.6 equiv), CuSO₄ (135.59 mg, 0.850 mmol, 1 equiv), NaN₃ (165.70 mg, 2.550 mmol, 3 equiv) and sodium ascorbate (338.33 mg, 1.700 mmol, 2 equiv). The reaction was stirred at 80° C. under N₂ for 3 h. After cooling down to rt, the reaction was quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give (2R)-7-amino-4-{[3-(difluoromethyl)phenyl]methyl}-6-fluoro-2-methyl-2H-1,4-benzoxazin-3-one (100 mg, 35.00%) as a yellow solid. LC/MS: mass calcd. for C₁₇H₁₅F₃N₂O₂:336.11 m/z, found: 337.10 [M+H]⁺.

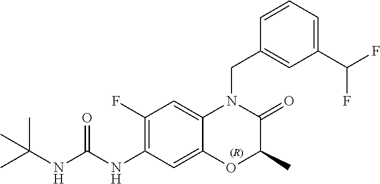

### (R)-1-(tert-butyl)-3-(4-(3-(difluoromethyl)benzyl)-6-fluoro-2-methyl-3-oxo-3,4-dihydro-2H-benzo[b][1,4]oxazin-7-yl)urea

To a solution of (2R)-7-amino-4-{[3-(difluoromethyl)phenyl]methyl}-6-fluoro-2-methyl-2H-1,4-benzoxazin-3-one (100 mg, 0.297 mmol, 1 equiv) in DCM (5 mL) and toluene (2.5 mL) was added 2-isocyanato-2-methylpropane (88.43 mg, 0.891 mmol, 3 equiv). The reaction was stirred at 90° C. under N₂ for 3 h. After cooling down to rt, the reaction was quenched with water (50 mL) and extracted with DCM (3×50 mL). The combined extracts were washed with water, brine, dried over Na₂SO₄, filtered and concentrated. The residue was purified by preparative HPLC using a XBridge Prep OBD C18 Column, 30*150 mm, 5 μm column (eluent: 42% to 64% (v/v) CH₃CN and Water (10 mmol/L NH₄HCO₃)) to afford the title compound 3-tert-butyl-1-[(2R)-4-{[3-(difluoromethyl)phenyl]methyl}-6-fluoro-2-methyl-3-oxo-2H-1,4-benzoxazin-7-yl]urea (22.9 mg, 17.69%) as a white solid. LC/MS: mass calcd. for C₂₂H₂₄F₃N₃O₃:435.18 m/z, found: 458.10 [M+Na]⁺. ¹H NMR (400 MHz, DMSO-d₆) δ 8.08 (d, J=2.6 Hz, 1H), 7.84 (d, J=7.7 Hz, 1H), 7.37-7.52 (m, 4H), 6.84-7.21 (m, 2H), 6.51 (s, 1H), 5.10-5.25 (m, 2H), 4.83 (q, J=6.7 Hz, 1H), 1.48 (d, J=6.8 Hz, 3H), 1.27 (s, 9H). ¹⁹F NMR (376 MHz, DMSO) δ(ppm): −109.7463, −137.0823.

### Example 328: 1-(tert-butyl)-3-((R)-6-fluoro-2-methyl-3-oxo-4-((S)-1-phenylethyl)-3,4-dihydro-2H-benzo[b][1,4]oxazin-7-yl)urea

### Synthetic Scheme

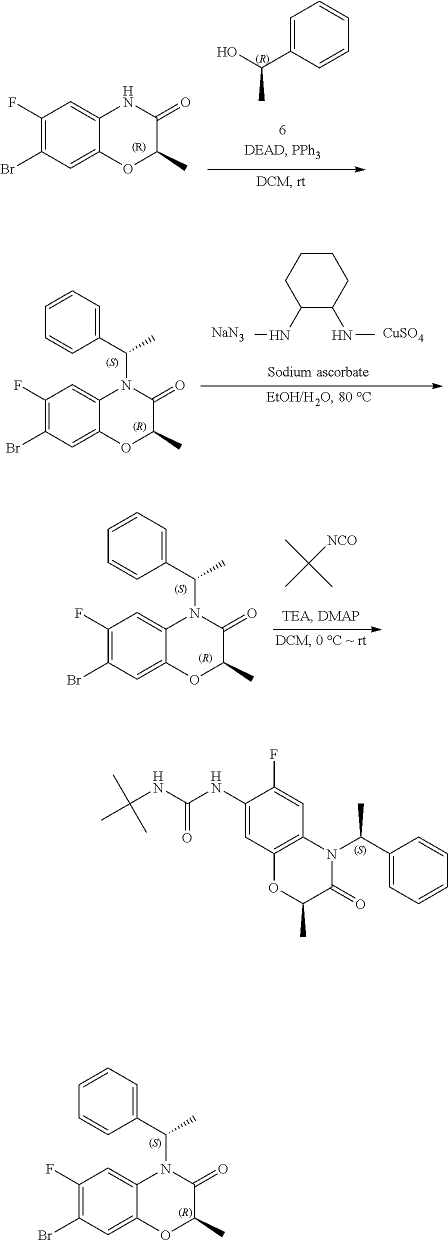

### (R)-7-bromo-6-fluoro-2-methyl-4-((S)-1-phenylethyl)-2H-benzo[b][1,4]oxazin-3(4H)-one

A solution of (2R)-7-bromo-6-fluoro-2-methyl-2,4-dihydro-1,4-benzoxazin-3-one (300 mg, 1.154 mmol, 1 equiv) in DCM (5 mL) was treated with 1-phenyl-ethanol (140.93 mg, 1.154 mmol, 1 equiv) and PPh₃ (453.86 mg, 1.731 mmol, 1.5 equiv) for 1 h at rt under nitrogen atmosphere followed by the addition of DEAD (200.90 mg, 1.154 mmol, 1 equiv) dropwise at 0° C. The resulting mixture was stirred for 2 h at rt under N₂ atmosphere. Quenched with water (10 mL) and extracted with DCM (3×10 mL). The combined extracts were washed with water, brine, dried over Na₂SO₄, filtered and concentrated. The residue was purified by column chromatography on silica gel with EA/PE (0-100%) to give (2R)-7-bromo-6-fluoro-2-methyl-4-[(1S)-1-phenylethyl]-2H-1,4-benzoxazin-3-one (195 mg, 46.41%) as a white solid. MS (ESI): mass calcd. for C₁₇H₁₅BrFNO₂: 363.03 m/z, found 364.10, 366.10 [M+H, M+H+2]⁺.

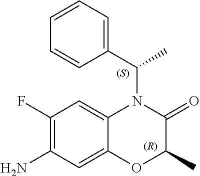

### (R)-7-amino-6-fluoro-2-methyl-4-((S)-1-phenylethyl)-2H-benzo[b][1,4]oxazin-3(4H)-one

To a solution of (2R)-7-bromo-6-fluoro-2-methyl-4-[(1S)-1-phenylethyl]-2H-1,4-benzoxazin-3-one (175 mg, 0.408 mmol, 1 equiv) in EtOH (4 mL)/H₂O (1 mL) was added N₁,N₂-dimethylcyclohexane-1,2-diamine (34.80 mg, 0.245 mmol, 0.6 equiv), CuSO₄ (65.07 mg, 0.408 mmol, 1 equiv), sodium ascorbate (162.36 mg, 0.816 mmol, 2 equiv) and NaN₃ (79.52 mg, 1.224 mmol, 3 equiv). The reaction was stirred at 80° C. for 3 h. The mixture was allowed to cool down to rt. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give (2R)-7-amino-6-fluoro-2-methyl-4-[(1S)-1-phenylethyl]-2H-1,4-benzoxazin-3-one (60 mg, 49.00%) as a white solid. MS (ESI): mass calcd. for C₁₇H₁₇FN₂O₂: 300.13 m/z, found 301.05 [M+H]⁺.

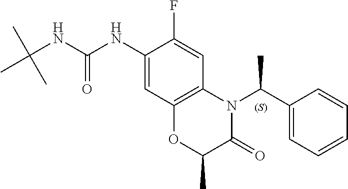

### 1-(tert-butyl)-3-((R)-6-fluoro-2-methyl-3-oxo-4-((S)-1-phenylethyl)-3,4-dihydro-2H-benzo[b][1,4]oxazin-7-yl)urea

To a solution of (2R)-7-amino-6-fluoro-2-methyl-4-[(1S)-1-phenylethyl]-2H-1,4-benzoxazin-3-one (60 mg, 0.200 mmol, 1 equiv) and 2-isocyanato-2-methylpropane (59.41 mg, 0.600 mmol, 3 equiv) in DCM (3 mL) was added TEA (60.65 mg, 0.600 mmol, 3 equiv) and DMAP (2.44 mg, 0.020 mmol, 0.1 equiv). The reaction was stirred at rt for 24 h. Quenched with water (10 mL) and extracted with DCM (3×10 mL). The combined extracts were washed with water, brine, dried over Na₂SO₄, filtered and concentrated. The residue was purified by preparative HPLC using a YMC-Actus Triart C18 ExRS 150 mm×30 mm×5 μm column (eluent: 49% to 73% (v/v) CH₃CN and H₂O with 10 mmol/L NH₄HCO₃) to afford the title compound 3-tert-butyl-1-[(2R)-6-fluoro-2-methyl-3-oxo-4-[(1S)-1-phenylethyl]-2H-1,4-benzoxazin-7-yl]urea (14.5 mg, 18.06%) as a white solid. LC/MS (ESI): mass calcd. for C₂₂H₂₆FN₃O₃:399.20 m/z, found: 400.10 [M+H]⁺. ¹H NMR (300 MHz, DMSO-d₆) δ 8.04 (s, 1H), 7.80-7.82 (d, J=6 Hz, 1H), 7.33-7.44 (m, 2H), 7.23-7.33 (m, 3H), 6.48-6.59 (m, 2H), 6.04-6.06 (m, 1H), 4.72-4.75 (m, 1H), 1.74-1.76 (d, J=6 Hz, 3H), 1.41-1.44 (d, J=9 Hz, 3H), 1.26 (s, 9H). ¹⁹F NMR (282 MHz, DMSO) δ−137.16.

### Example 329: (R)-1-(4-benzyl-6,8-difluoro-2-methyl-3-oxo-3,4-dihydro-2H-benzo[b][1,4]oxazin-7-yl)-3-(tert-butyl)urea

### Synthetic Scheme

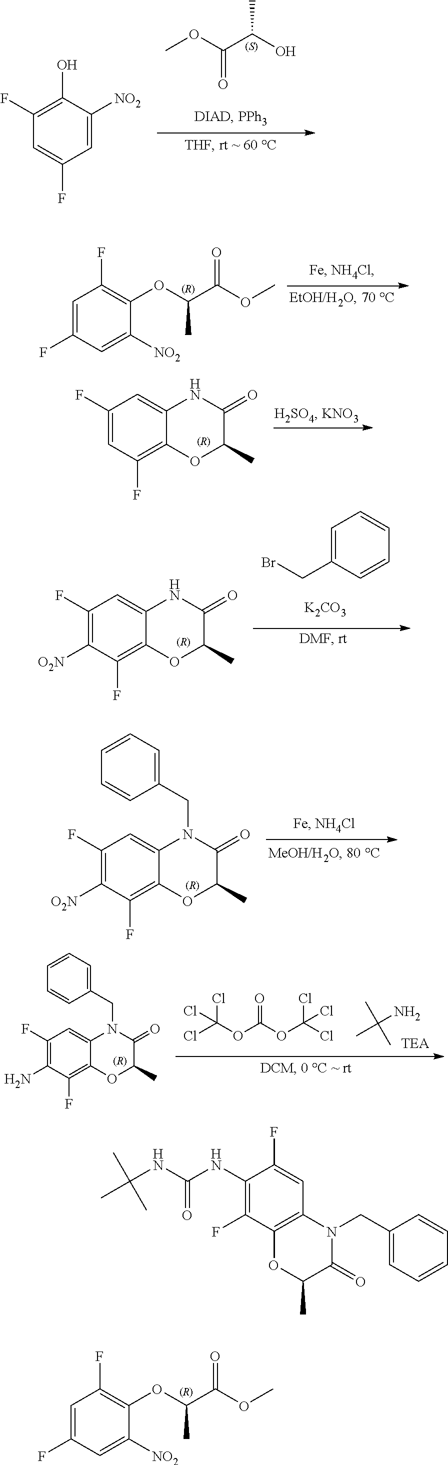

### methyl (R)-2-(2,4-difluoro-6-nitrophenoxy)propanoate

To a solution of 2,4-difluoro-6-nitrophenol (2 g, 11.423 mmol, 1 equiv) in THF (20 mL) was added methyl (2S)-2-hydroxypropanoate (1.19 g, 11.423 mmol, 1 equiv) and PPh₃ (4.49 g, 17.134 mmol, 1.5 equiv). The reaction was stirred at rt under N₂ for 0.5 h. To the above mixture was added DIAD (2.21 g, 17.134 mmol, 1.5 equiv) at 0° C. The resulting mixture was stirred for additional 1 h at 60° C. The reaction was monitored by TLC. After cooling down to rt, the reaction was quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give methyl (2R)-2-(2,4-difluoro-6-nitrophenoxy)propanoate (2.5 g, 83.80%) as a white oil. LC/MS: mass calcd. for C₁₀H₉F₂NO₅:261.04 m/z.

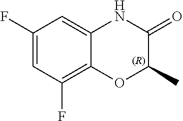

### (R)-6,8-difluoro-2-methyl-2H-benzo[b][1,4]oxazin-3(4H)-one

To a solution of methyl (2R)-2-(2,4-difluoro-6-nitrophenoxy)propanoate (2.5 g, 9.572 mmol, 1 equiv) in EtOH (30 mL) and H₂O (3 mL) was added Fe (5.35 g, 95.720 mmol, 10 equiv) and NH₄C₁ (5.12 g, 95.720 mmol, 10 equiv). The reaction was stirred at 80° C. for 1 h. The mixture was allowed to cool down to rt. The resulting mixture was filtered, the filter cake was washed with EtOH (3×100 mL). The filtrate was concentrated under reduced pressure. Diluted with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give (2R)-6,8-difluoro-2-methyl-2,4-dihydro-1,4-benzoxazin-3-one (1.74 g, 91.28%) as white oil. LC/MS: mass calcd. for C₉H₇F₂NO₂:199.04 m/z, found: 197.90 [M−H]⁻.

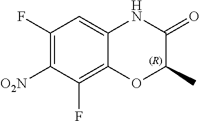

### (R)-6,8-difluoro-2-methyl-7-nitro-2H-benzo[b][1,4]oxazin-3(4H)-one

To a solution of (2R)-6,8-difluoro-2-methyl-2,4-dihydro-1,4-benzoxazin-3-one (800 mg, 4.017 mmol, 1 equiv) in H₂SO₄ (10 mL) was added KNO₃ (406.12 mg, 4.017 mmol, 1 equiv) at 0° C. The reaction was stirred at rt for 1 h. The reaction was quenched with water/Ice at 0° C. Diluted with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give (2R)-6,8-difluoro-2-methyl-7-nitro-2,4-dihydro-1,4-benzoxazin-3-one (900 mg, 91.77%) as a yellow solid. LC/MS: mass calcd. for C₉H₆F₂N₂O₄:244.03 m/z, found: 242.95 [M−H]⁻.

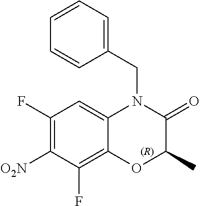

### (R)-4-benzyl-6,8-difluoro-2-methyl-7-nitro-2H-benzo[b][1,4]oxazin-3(4H)-one

To a solution of (2R)-6,8-difluoro-2-methyl-7-nitro-2,4-dihydro-1,4-benzoxazin-3-one (300 mg, 1.229 mmol, 1 equiv) in DMF (10 mL) was added K₂CO₃ (513.17 mg, 3.687 mmol, 3 equiv) and benzyl bromide (315.24 mg, 1.844 mmol, 1.5 equiv) at 0° C. The reaction was stirred at rt for 1 h. The reaction was monitored by TLC. Quenched with water (30 mL) and extracted with EA (3×30 mL). The combined extracts were washed with water, brine, dried over Na₂SO₄, filtered and concentrated. The residue was purified by column chromatography on silica gel with EA/PE (0-100%) to give (2R)-4-benzyl-6,8-difluoro-2-methyl-7-nitro-2H-1,4-benzoxazin-3-one (390 mg, 94.95%) as a yellow solid.

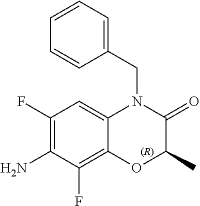

### (R)-7-amino-4-benzyl-6,8-difluoro-2-methyl-2H-benzo[b][1,4]oxazin-3(4H)-one

To a solution of (2R)-4-benzyl-6,8-difluoro-2-methyl-7-nitro-2H-1,4-benzoxazin-3-one (380 mg, 1.137 mmol, 1 equiv) in MeOH (10 mL) and H₂O (1 mL) was added Fe (634.83 mg, 11.370 mmol, 10 equiv) and NH₄C₁ (608.06 mg, 11.370 mmol, 10 equiv). The reaction was stirred at 80° C. for 1 h. The mixture was allowed to cool down to rt. The resulting mixture was filtered, the filter cake was washed with MeOH (3×50 mL). The filtrate was concentrated under reduced pressure. Quenched with water (30 mL) and extracted with EA (3×30 mL). The combined extracts were washed with water, brine, dried over Na₂SO₄, filtered and concentrated. The residue was purified by column chromatography on silica gel with EA/PE (0-100%) to give (2R)-7-amino-4-benzyl-6,8-difluoro-2-methyl-2H-1,4-benzoxazin-3-one (320 mg, 92.51%) as a yellow solid. LC/MS: mass calcd. for C₁₆H₁₄F₂N₂O₂: 304.10 m/z, found: 305.15 [M+H]⁺.

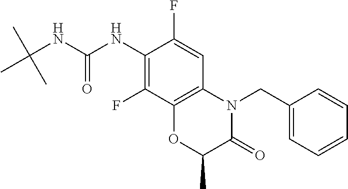 [SMILES: C[C@H]1Oc2c(cc(F)c(NC(=O)NC(C)(C)C)c2F)N(Cc2ccccc2)C1=O]

### (R)-1-(4-benzyl-6,8-difluoro-2-methyl-3-oxo-3,4-dihydro-2H-benzo[b][1,4]oxazin-7-yl)-3-(tert-butyl)urea

To a solution of (2R)-7-amino-4-benzyl-6,8-difluoro-2-methyl-2H-1,4-benzoxazin-3-one (280 mg, 0.920 mmol, 1 equiv) in DCM (5 mL) was added triphosgene (109.21 mg, 0.368 mmol, 0.4 equiv) under N₂ at 0° C. The reaction was stirred at 0° C. under N₂ for 1 h. To the above mixture was added TEA (279.34 mg, 2.760 mmol, 3 equiv) at 0° C. The resulting mixture was stirred for additional 1 h at rt. Then, to the above mixture was added erbumine (201.90 mg, 2.760 mmol, 3 equiv). The resulting mixture was stirred for additional 1 h at rt. Quenched with water (10 mL) and extracted with DCM (3×10 mL). The combined extracts were washed with water, brine, dried over Na₂SO₄, filtered and concentrated. The residue was purified by preparative HPLC using a XBridge Prep OBD C18 Column, 30*150 mm, 5 μm column (eluent: 36% to 66% (v/v) CH₃CN and Water (10 mmol/L NH₄HCO₃)) to afford the title compound 1-[(2R)-4-benzyl-6,8-difluoro-2-methyl-3-oxo-2H-1,4-benzoxazin-7-yl]-3-tert-butylurea (65.3 mg, 17.26%) as a white solid. LC/MS: mass calcd. for C₂₁H₂₃F₂N₃O₃:403.17 m/z, found: 404.05 [M+H]⁺. ¹H NMR (400 MHz, DMSO-d₆) δ 7.50 (s, 1H), 7.31-7.39 (m, 2H), 7.26 (td, J=5.2, 3.0 Hz, 3H), 6.90 (dd, J=11.4, 1.9 Hz, 1H), 6.15 (s, 1H), 5.09-5.23 (m, 2H), 4.98 (q, J=6.7 Hz, 1H), 1.54 (d, J=6.7 Hz, 3H), 1.25 (s, 9H). ¹⁹F NMR (376 MHz, DMSO) δ(ppm): −126.0506, −139.2961.

### Example 330: (R)-1-(4-benzyl-2-methyl-3-oxo-6-(trifluoromethyl)-3,4-dihydro-2H-benzo[b][1,4]oxazin-7-yl)-3-(tert-butyl)urea

### Synthetic Scheme

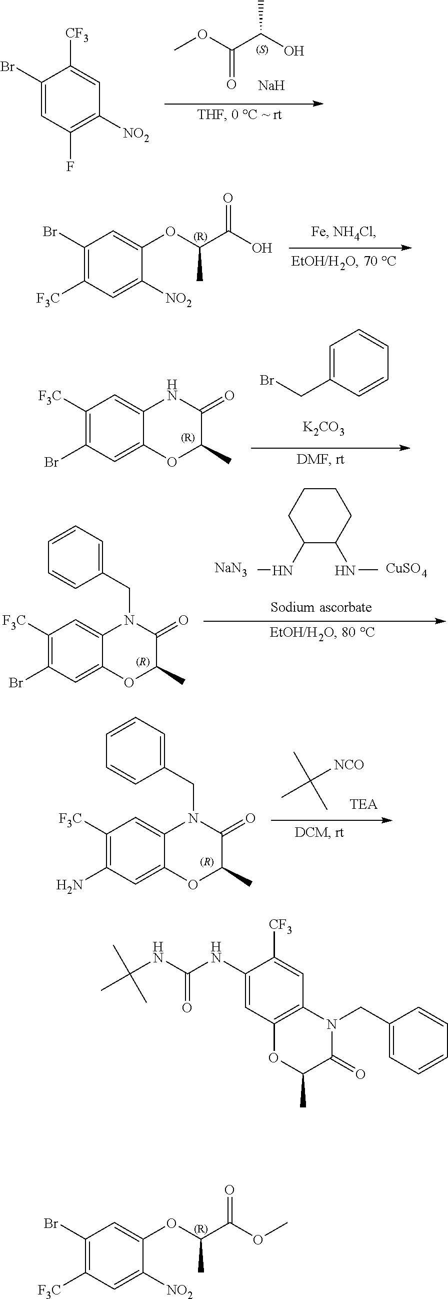

### methyl (R)-2-(5-bromo-2-nitro-4-(trifluoromethyl)phenoxy)propanoate

To a stirred solution of methyl (2S)-2-hydroxypropanoate (181 mg, 1.736 mmol, 1 equiv) in THF was added 60% NaH (83.3 mg, 2.083 mmol, 1.2 equiv) at 0° C. The final reaction mixture was stirred for 30 min at 0° C. To the above mixture was added 1-bromo-5-fluoro-4-nitro-2-(trifluoromethyl)benzene (500 mg, 1.736 mmol, 1 equiv) at room temperature. The reaction was stirred for 2 h at rt. The reaction was quenched with water (10 mL). The aqueous layer was extracted with EtOAc (3×10 mL). The residue was purified by silica gel column chromatography, eluted with PE/EA (1:1) to afford methyl (R)-2-(5-bromo-2-nitro-4-(trifluoromethyl)phenoxy)propanoate (410 mg, 63.47%) as a white solid. (ESI): mass calcd. for C₁₁H₉BrF₃NO₅: 370.96 m/z, found: 372.09 [M+H]⁺.

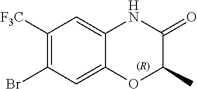

### (R)-7-bromo-2-methyl-6-(trifluoromethyl)-2H-benzo[b][1,4]oxazin-3(4H)-one

To a mixture of methyl (R)-2-(5-bromo-2-nitro-4-(trifluoromethyl)phenoxy)propanoate (0.4 g, 1.075 mmol, 1 equiv) in EtOH (25 mL) and water (5 mL). Fe (0.6 g, 10.75 mmol, 10 equiv) and NH₄Cl (569 mg, 10.75 mmol, 10 equiv) was added into the mixture. The resulting solution was stirred at 70° C. for 1 h. The reaction was quenched with water (30 mL) at rt. After filtration, the filter cake was washed with EA (3×30 mL). The filtrate was added water (30 mL) and extracted with EA (3×50 mL). The combined organic layers were washed with brine (30 mL), dried over anhydrous Na₂SO₄. After filtration, the filtrate was concentrated under reduced pressure. The crude product was purified by silica gel column chromatography, eluted with PE/EA (1:1) to afford (R)-7-bromo-2-methyl-6-(trifluoromethyl)-2H-benzo[b][1,4]oxazin-3(4H)-one as a light yellow oil. (ESI): mass calcd. for C₁₀H₇BrF₃NO₂: 308.96 m/z, found: 310.07 [M+H]⁺.

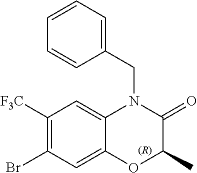

### (R)-4-benzyl-7-bromo-2-methyl-6-(trifluoromethyl)-2H-benzo[b][1,4]oxazin-3(4H)-one

A solution of (R)-7-bromo-2-methyl-6-(trifluoromethyl)-2H-benzo[b][1,4]oxazin-3(4H)-one (300 mg, 0.968 mmol, 1 equiv) and benzyl bromide (248 mg, 1.452 mmol, 1.5 equiv) and K₂CO₃ (404 mg, 2.904 mmol, 3 equiv) in DMF was stirred for 3 h at room temperature. The reaction was quenched with water (10 mL) at room temperature. The aqueous layer was extracted with EtOAc (3×10 mL). The residue was purified by silica gel column chromatography, eluted with PE/EA (1:1) to afford (R)-4-benzyl-7-bromo-2-methyl-6-(trifluoromethyl)-2H-benzo[b][1,4]oxazin-3(4H)-one (310 mg, 80.06%) as a white solid. (ESI): mass calcd. for C₁₇H₁₃BrF₃NO₂: 399.01 m/z, found: 400.19 [M+H]⁺.

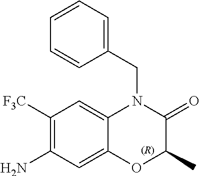

### (R)-7-amino-4-benzyl-2-methyl-6-(trifluoromethyl)-2H-benzo[b][1,4]oxazin-3(4H)-one

To a solution of (R)-4-benzyl-7-bromo-2-methyl-6-(trifluoromethyl)-2H-benzo[b][1,4]oxazin-3(4H)-one (279 mg, 0.699 mmol, 1 equiv) in EtOH (6 mL) & H₂O (2 mL) was added sodium ascorbate (28 mg, 0.140 mmol, 0.2 equiv), N¹,N²-dimethylcyclohexane-1,2-diamine (20 mg, 0.140 mmol, 0.2 equiv), CuSO₄ (23 mg, 0.14 mmol, 0.2 equiv) and NaN₃ (136 mg, 2.097 mmol, 3 equiv) at rt. The resulting mixture was stirred 4 h at 80° C. The reaction mixture was quenched by water (10 mL) and extracted with EA (3×30 mL). The combined organic extracts were washed with brine (30 mL), dried over anhydrous Na₂SO₄ and concentrated under vacuum. The crude product was purified by flash chromatography with PE/EA (1:1) to afford (R)-7-amino-4-benzyl-2-methyl-6-(trifluoromethyl)-2H-benzo[b][1,4]oxazin-3(4H)-one (177 mg, 75.51%) as a yellow oil. (ESI): mass calcd. for C₁₇H₁₅F₃N₂O₂: 336.10 m/z, found: 337.11 [M+H]⁺.

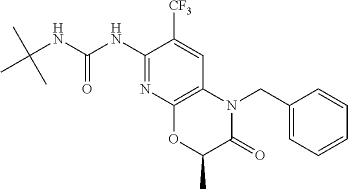 [SMILES: C[C@H]1Oc2nc(NC(=O)NC(C)(C)C)c(C(F)(F)F)cc2N(Cc2ccccc2)C1=O]

### (R)-1-(4-benzyl-2-methyl-3-oxo-6-(trifluoromethyl)-3,4-dihydro-2H-benzo[b][1,4]oxazin-7-yl)-3-(tert-butyl)urea

To a mixture of 2-(1-(7-amino-3-oxo-2,3-dihydro-4H-benzo[b][1,4]oxazin-4-yl)ethyl)-4-chlorobenzonitrile (34 mg, 0.101 mmol, 1 equiv) and TEA (21 mg, 0.202 mmol, 2 equiv) in DCM (4 mL) was added 2-isocyanato-2-methylpropane (30 mg, 0.303 mmol, 3 equiv) at 0° C. The reaction was stirred for overnight at rt. The reaction mixture was quenched by water (10 mL) and extracted with DCM (3×10 mL). The organic layers were combined, dried over anhydrous sodium sulfate, filtered and concentrated. The crude product was purified by silica gel column with PE/EA (1:1) and Prep-HPLC (Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 50% B to 72% B in 7 min; Wave Length: 254 nm/220 nm nm; RT1(min): 6.07) to afford (R)-1-(4-benzyl-2-methyl-3-oxo-6-(trifluoromethyl)-3,4-dihydro-2H-benzo[b][1,4]oxazin-7-yl)-3-(tert-butyl)urea (15 mg, 34.09%) as a white solid. (ESI): mass calcd. for C₂₂H₂₄F₃N₃O₃: 435.18 m/z, found: 436.18 [M+H]⁺. ¹H NMR (400 MHz, DMSO-d₆) δ 7.69 (s, 1H), 7.57 (s, 1H), 7.33-7.41 (m, 2H), 7.21-7.30 (m, 3H), 7.12 (s, 1H), 6.94 (s, 1H), 5.13-5.28 (m, 2H), 4.92-5.03 (m, 1H), 1.51-1.59 (m, 3H), 1.27 (s, 9H).

### Example 331: 1-(tert-butyl)-3-((R)-2-methyl-3-oxo-4-((R)-1-phenylethyl)-3,4-dihydro-2H-benzo[b][1,4]oxazin-7-yl)urea

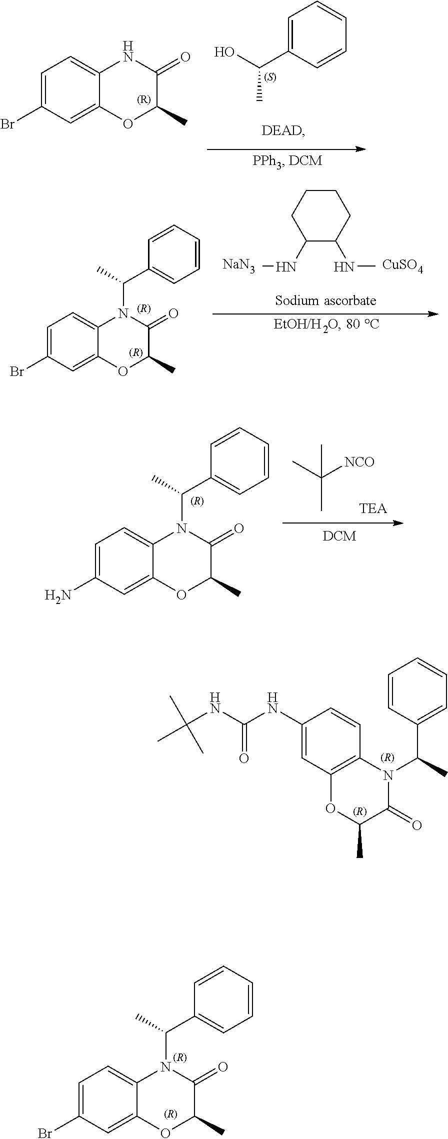

### (R)-7-bromo-2-methyl-4-((R)-1-phenylethyl)-2H-benzo[b][1,4]oxazin-3(4H)-one

A solution of (2R)-7-bromo-2-methyl-2,4-dihydro-1,4-benzoxazin-3-one (600 mg, 2.479 mmol, 1 equiv) in DCM (10 mL) was treated with (S)-1-phenylethanol (454.20 mg, 3.719 mmol, 1.5 equiv) and PPh₃ (975.18 mg, 3.719 mmol, 1.5 equiv) for 1 h at rt under nitrogen atmosphere followed by the addition of DEAD (647.49 mg, 3.719 mmol, 1.5 equiv) dropwise at 0° C. The resulting mixture was stirred for 2 h at rt under N₂ atmosphere. Quenched with water (10 mL) and extracted with DCM (3×10 mL). The combined extracts were washed with water, brine, dried over Na₂SO₄, filtered and concentrated. The residue was purified by column chromatography on silica gel with EA/PE (0-100%) to give (2R)-7-bromo-2-methyl-4-[(1R)-1-phenylethyl]-2H-1,4-benzoxazin-3-one (400 mg, 46.61%) as a white solid. MS (ESI): mass calcd. for C₁₇H₁₆BrNO₂: 345.04 m/z, found 346.00, 348.00 [M+H, M+H+2]⁺.

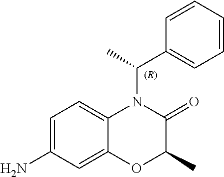

### (R)-7-amino-2-methyl-4-((R)-1-phenylethyl)-2H-benzo[b][1,4]oxazin-3(4H)-one

To a solution of (2R)-7-bromo-2-methyl-4-[(1R)-1-phenylethyl]-2H-1,4-benzoxazin-3-one (390 mg, 0.948 mmol, 1 equiv) in EtOH (4 mL) was added N₁,N₂-dimethylcyclohexane-1,2-diamine (80.94 mg, 0.569 mmol, 0.6 equiv), CuSO₄ (151.36 mg, 0.948 mmol, 1 equiv), sodium ascorbate (377.66 mg, 1.896 mmol, 2 equiv) and NaN₃ (184.96 mg, 2.844 mmol, 3 equiv). The reaction was stirred at 80° C. under N₂ for 1 h. The mixture was allowed to cool down to rt. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give (2R)-7-amino-2-methyl-4-[(1R)-1-phenylethyl]-2H-1,4-benzoxazin-3-one (250 mg, 93.37%) as a white solid. MS (ESI): mass calcd. for C₁₇H₁₈N₂O₂: 282.14 m/z, found 283.10 [M+H]⁺.

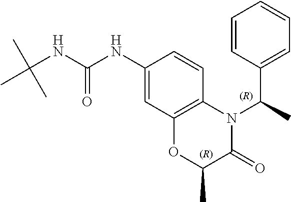

### 1-(tert-butyl)-3-((R)-2-methyl-3-oxo-4-((R)-1-phenylethyl)-3,4-dihydro-2H-benzo[b][1,4]oxazin-7-yl)urea

To a solution of (2R)-7-amino-2-methyl-4-[(1R)-1-phenylethyl]-2H-1,4-benzoxazin-3-one (294 mg, 1.041 mmol, 1 equiv) and 2-isocyanato-2-methylpropane (309.68 mg, 3.123 mmol, 3 equiv) in DCM (10 mL) was added TEA (316.11 mg, 3.123 mmol, 3 equiv) and DMAP (12.72 mg, 0.104 mmol, 0.1 equiv). The reaction was stirred at rt for 24 h. Quenched with water (20 mL) and extracted with DCM (3×20 mL). The combined extracts were washed with water, brine, dried over Na₂SO₄, filtered and concentrated. The residue was purified by preparative HPLC using a YMC-Actus Triart C18 ExRS 150 mm×30 mm×5 μm column (eluent: 49% to 73% (v/v) CH₃CN and H₂O with 10 mmol/L NH₄HCO₃) to afford the title compound 3-tert-butyl-1-[(2R)-2-methyl-3-oxo-4-[(1R)-1-phenylethyl]-2H-1,4-benzoxazin-7-yl]urea (58.6 mg, 14.53%) as a white solid. LC/MS (ESI): mass calcd. for C₂₂H₂₇N₃O₃:381.21 m/z, found: 382.10 [M+H]⁺. ¹H NMR (300 MHz, DMSO-d₆) δ 8.18 (s, 1H), 7.25-7.39 (m, 6H), 6.62-6.63 (m, 1H), 6.60-6.61 (m, 1H), 6.15-6.17 (m, 1H), 5.94 (s, 1H), 4.69-4.75 (m, 1H), 1.66-1.69 (d, J=9.0 Hz, 3H), 1.46-1.48 (d, J=6.0 Hz, 3H), 1.26 (s, 9H).

### Example 332: (R)-1-(4-benzyl-2,6-dimethyl-3-oxo-3,4-dihydro-2H-benzo[b][1,4]oxazin-7-yl)-3-(tert-butyl)urea

### Synthetic Scheme

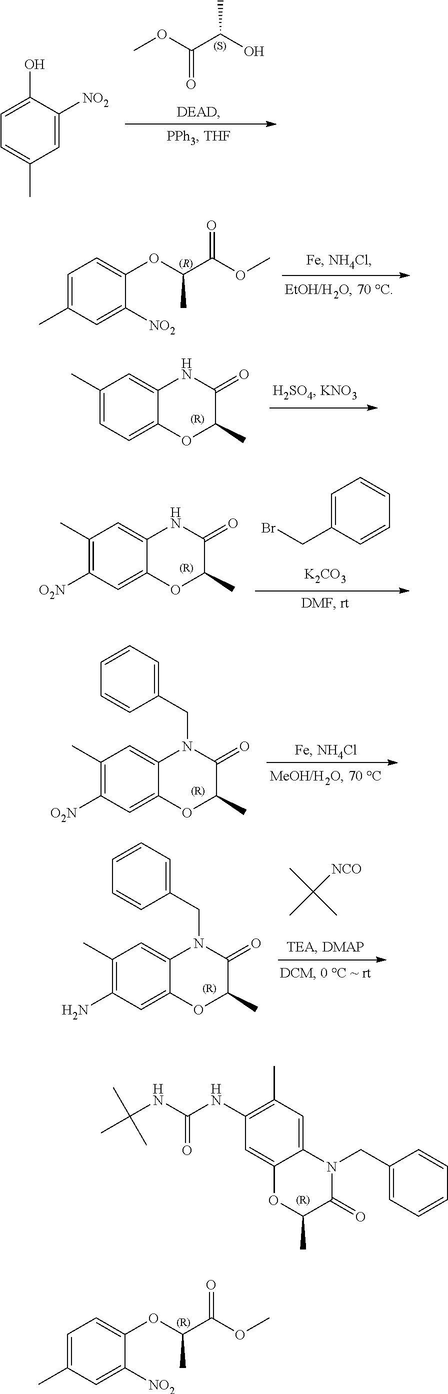

### methyl (2R)-2-(4-methyl-2-nitrophenoxy)propanoate

To a solution of 2-nitro-P-cresol (1 g, 6.530 mmol, 1 equiv) in THF (15 mL) was added methyl (2S)-2-hydroxypropanoate (0.82 g, 7.836 mmol, 1.2 equiv) and PPh₃ (2.57 g, 9.795 mmol, 1.5 equiv) under N₂ at 0° C. The reaction was stirred at 0 degrees C. for 0.5 h. Then DEAD (1.71 g, 9.795 mmol, 1.5 equiv) was added at 0° C. The reaction was stirred at 60 degrees C. for 2 h. The mixture was allowed to cool down to room temperature. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give methyl (2R)-2-(4-methyl-2-nitrophenoxy)propanoate (1.3 g, 83.22%) as a yellow solid. LC/MS (ESI): mass calcd. for C₁₁H₁₃NO₅:239.08 m/z, found: 240.05 [M+H]⁺.

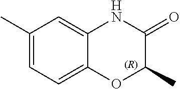

### (2R)-2,6-dimethyl-2,4-dihydro-1,4-benzoxazin-3-one

To a solution of methyl (2R)-2-(4-methyl-2-nitrophenoxy)propanoate (700 mg, 2.926 mmol, 1 equiv) in EtOH (10 mL) and H₂O (2 mL) was added Fe (1.63 g, 29.260 mmol, 10 equiv) and NH₄Cl (1.57 g, 29.260 mmol, 10 equiv). The reaction was stirred at 70 degrees C. for 2 h. The mixture was allowed to cool down to room temperature. The resulting mixture was filtered, the filter cake was washed with DCM (3×50 mL). The filtrate was concentrated under reduced pressure. Quenched with water (100 mL) and extracted with DCM (3×100 mL). The combined extracts were washed with water, brine, dried over Na₂SO₄, filtered and concentrated. The residue was purified by column chromatography on silica gel with EA/PE (0-100%) to give (2R)-2,6-dimethyl-2,4-dihydro-1,4-benzoxazin-3-one (450 mg, 86.79%) as a yellow solid. LC/MS (ESI): mass calcd. for C₁₀H₁₁NO₂:177.08 m/z, found: 178.15 [M+H]⁺.

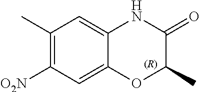

### (2R)-2,6-dimethyl-7-nitro-2,4-dihydro-1,4-benzoxazin-3-one

To a solution of (2R)-2,6-dimethyl-2,4-dihydro-1,4-benzoxazin-3-one (420 mg, 2.370 mmol, 1 equiv) in H₂SO₄ (5 mL) was added KNO₃ (311.52 mg, 3.081 mmol, 1.3 equiv) at 0° C. The reaction was stirred at 0 degrees C. for 1 h. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give (2R)-2,6-dimethyl-7-nitro-2,4-dihydro-1,4-benzoxazin-3-one (160 mg, 30.38%) as a yellow solid. TLC showed the desired product was generated.

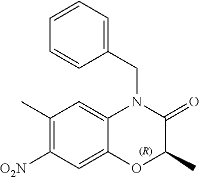

### (2R)-4-benzyl-2,6-dimethyl-7-nitro-2H-1,4-benzoxazin-3-one

To a solution of (2R)-2,6-dimethyl-7-nitro-2,4-dihydro-1,4-benzoxazin-3-one (160 mg, 0.720 mmol, 1 equiv) in DMF (5 mL) was added benzyl bromide (160.11 mg, 0.936 mmol, 1.3 equiv) and K₂CO₃ (300.73 mg, 2.160 mmol, 3 equiv). The reaction was stirred at 25 degrees C. for 16 h. Quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give (2R)-4-benzyl-2,6-dimethyl-7-nitro-2H-1,4-benzoxazin-3-one (200 mg, 88.93%) as a yellow solid. LC/MS (ESI): mass calcd. for C₁₇H₁₆N₂O₄:312.11 m/z, found: 310.95 [M−H]⁻.

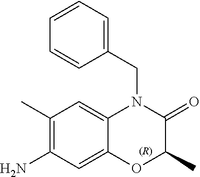

### (2R)-7-amino-4-benzyl-2,6-dimethyl-2H-1,4-benzoxazin-3-one

To a solution of (2R)-4-benzyl-2,6-dimethyl-7-nitro-2H-1,4-benzoxazin-3-one (190 mg, 0.608 mmol, 1 equiv) in MeOH (4 mL) and H₂O (1 mL) was added Fe (339.73 mg, 6.080 mmol, 10 equiv) and NH₄Cl (325.40 mg, 6.080 mmol, 10 equiv). The reaction was stirred at 70 degrees C. for 2 h. The mixture was allowed to cool down to room temperature. The resulting mixture was filtered, the filter cake was washed with DCM (3×50 mL). The filtrate was concentrated under reduced pressure. Diluted with water (40 mL) and extracted with DCM (3×40 mL). The combined extracts were washed with water, brine, dried over Na₂SO₄, filtered and concentrated. The residue was purified by column chromatography on silica gel with EA/PE (0-100%) to give (2R)-7-amino-4-benzyl-2,6-dimethyl-2H-1,4-benzoxazin-3-one (130 mg, 75.69%) as a yellow solid. LC/MS (ESI): mass calcd. for C₁₇H₁₈N₂O₂:282.14 m/z, found: 283.10 [M+H]⁺.

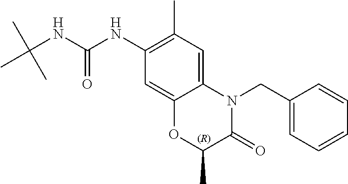

### (R)-1-(4-benzyl-2,6-dimethyl-3-oxo-3,4-dihydro-2H-benzo[b][1,4]oxazin-7-yl)-3-(tert-butyl)urea

To a solution of (R)-7-amino-4-benzyl-2,6-dimethyl-2H-benzo[b][1,4]oxazin-3(4H)-one (150 mg, 0.531 mmol, 1 equiv) in DCM (5 mL) was added NEt₃ (322.61 mg, 3.186 mmol, 6 equiv), 2-isocyanato-2-methylpropane (210.69 mg, 2.124 mmol, 4 equiv) and DMAP (6.49 mg, 0.053 mmol, 0.1 equiv) at 0° C. The reaction was stirred at 25° C. for 48 hours. Quenched with water (20 mL) and extracted with DCM (3×20 mL). The combined extracts were washed with water, brine, dried over Na₂SO₄, filtered and concentrated. The residue was purified by preparative HPLC using a XBridge C18 Column 250 mm×19 mm×5 μm column (eluent: 53% to 80% (v/v) CH₃CN and H₂O with 10 mmol/L NH₄HCO₃) to give the (R)-1-(4-benzyl-2,6-dimethyl-3-oxo-3,4-dihydro-2H-benzo[b][1,4]oxazin-7-yl)-3-(tert-butyl)urea (63.1 mg, 30.91%) as a white solid. MS (ESI): mass calcd. for C₂₂H₂₇N₃O₃: 381.2 m/z, found 382.20 [M+H]⁺. ¹H NMR (400 MHz, DMSO-d₆) δ 7.65 (s, 1H), 7.40 (s, 1H), 7.30-7.36 (m, 2H), 7.21-7.27 (m, 3H), 6.82 (s, 1H), 6.48 (s, 1H), 5.10 (s, 2H), 4.76 (q, J=6.7 Hz, 1H), 2.03 (s, 3H), 1.46 (d, J=6.7 Hz, 3H), 1.28 (s, 9H).

### Example 333 and Example 334: (S)-1-(1-benzyl-7-fluoro-3-methyl-2-oxo-1,2,3,4-tetrahydroquinolin-6-yl)-3-(tert-butyl)urea and (R)-1-(1-benzyl-7-fluoro-3-methyl-2-oxo-1,2,3,4-tetrahydroquinolin-6-yl)-3-(tert-butyl)urea

### Synthetic Scheme

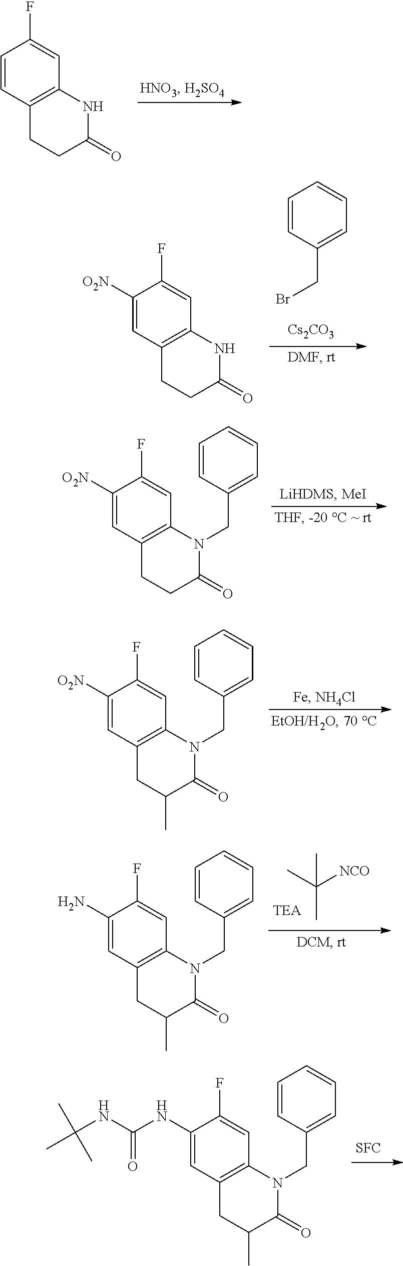

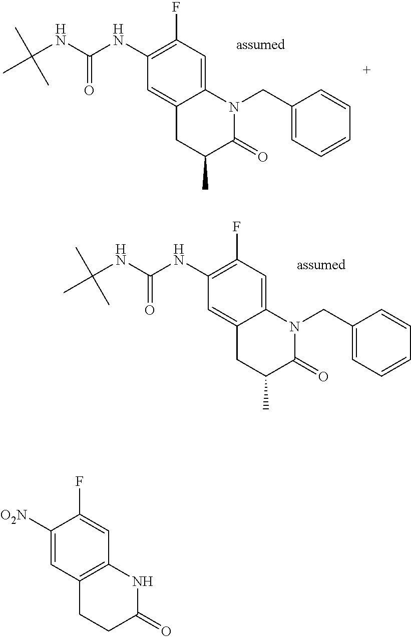

### 7-fluoro-6-nitro-3,4-dihydroquinolin-2(1H)-one

To a solution of 7-fluoro-3,4-dihydro-1H-quinolin-2-one (1 g, 6.054 mmol, 1 equiv) in H₂SO₄ (10 mL) was added HNO₃ (0.38 g, 6.054 mmol, 1 equiv) at 0° C. The reaction was stirred at 0° C. for 1 h. Quenched with ice water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 7-fluoro-6-nitro-3,4-dihydro-1H-quinolin-2-one (1.1 g, 86.45%) as a white solid. MS (ESI): mass calcd. for C₉H₇FN₂O₃: 210.04 m/z, found 209.05 [M−H]⁻.

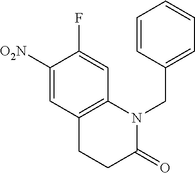 [SMILES: O=C1CCc2cc([N+](=O)[O-])c(F)cc2N1Cc1ccccc1]

### 1-benzyl-7-fluoro-6-nitro-3,4-dihydroquinolin-2(1H)-one

To a solution of 7-fluoro-6-nitro-3,4-dihydro-1H-quinolin-2-one (1.1 g, 5.234 mmol, 1 equiv) in DMF (10 mL) was added benzyl bromide (2.68 g, 15.702 mmol, 3 equiv) and Cs₂CO₃(5.13 g, 15.702 mmol, 3 equiv). The reaction was stirred at rt for 3 h. Quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give 1-benzyl-7-fluoro-6-nitro-3,4-dihydroquinolin-2-one (1.4 g, 89.07%) as a white solid. MS (ESI): mass calcd. for C₁₆H₁₃FN₂O₃: 300.09 m/z, found 300.95 [M+H]⁺.

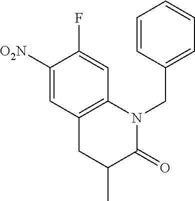 [SMILES: CC1Cc2cc([N+](=O)[O-])c(F)cc2N(Cc2ccccc2)C1=O]

### 1-benzyl-7-fluoro-3-methyl-6-nitro-3,4-dihydroquinolin-2(1H)-one

A solution of 1-benzyl-7-fluoro-6-nitro-3,4-dihydroquinolin-2-one (600 mg, 1.998 mmol, 1 equiv) in tetrahydrofuran (6 mL) was treated with LiHDMS (401.20 mg, 2.398 mmol, 1.2 equiv) for 1 h at −78° C. under nitrogen atmosphere followed by the addition of MeI (283.60 mg, 1.998 mmol, 1 equiv) dropwise at −78° C. The resulting mixture was stirred for 1 h at rt under N₂ atmosphere. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give 1-benzyl-7-fluoro-3-methyl-6-nitro-3,4-dihydroquinolin-2-one (275 mg, 43.79%) as a white solid. MS (ESI): mass calcd. for C₁₇H₁₅FN₂O₃: 314.11 m/z, found 315.10 [M+H]⁺.

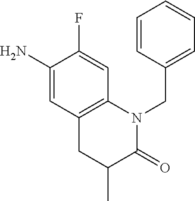 [SMILES: CC1Cc2cc(N)c(F)cc2N(Cc2ccccc2)C1=O]

### 6-amino-1-benzyl-7-fluoro-3-methyl-3,4-dihydroquinolin-2(1H)-one

To a solution of 1-benzyl-7-fluoro-3-methyl-6-nitro-3,4-dihydroquinolin-2-one (270 mg, 0.859 mmol, 1 equiv) in EtOH (5 mL)/H₂O (0.5 mL) was added Fe (479.71 mg, 8.590 mmol, 10 equiv) and NH₄C₁ (459.48 mg, 8.590 mmol, 10 equiv). The reaction was stirred at 70° C. for 3 h. The mixture was allowed to cool down to rt. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give 6-amino-1-benzyl-7-fluoro-3-methyl-3,4-dihydroquinolin-2-one (220 mg, 90.07%) as a white solid. MS (ESI): mass calcd. for C₁₇H₁₇FN₂O: 284.13 m/z, found 285.10 [M+H]⁺.

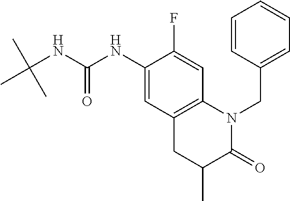 [SMILES: CC1Cc2cc(NC(=O)NC(C)(C)C)c(F)cc2N(Cc2ccccc2)C1=O]

### 1-(1-benzyl-7-fluoro-3-methyl-2-oxo-1,2,3,4-tetrahydroquinolin-6-yl)-3-(tert-butyl)urea

To a solution of 6-amino-1-benzyl-7-fluoro-3-methyl-3,4-dihydroquinolin-2-one (210 mg, 0.739 mmol, 1 equiv) in DCM (5 mL) was added 2-isocyanato-2-methylpropane (219.65 mg, 2.217 mmol, 3 equiv) and TEA (224.21 mg, 2.217 mmol, 3 equiv). The reaction was stirred at rt for 24 h. Quenched with water (10 mL) and extracted with DCM (3×10 mL). The combined extracts were washed with water, brine, dried over Na₂SO₄, filtered and concentrated. The residue was purified by column chromatography on silica gel with EA/PE (0-100%) to give 1-(1-benzyl-7-fluoro-3-methyl-2-oxo-3,4-dihydroquinolin-6-yl)-3-tert-butylurea (70 mg, 24.72%) as a white solid. MS (ESI): mass calcd. for C₂₂H₂₆FN₃O₂: 383.20 m/z, found 382.00 [M−H]⁻.

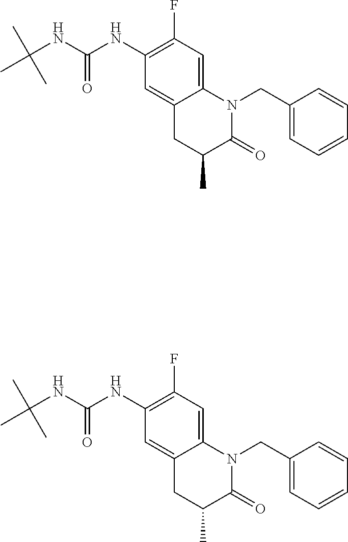 [SMILES: C[C@H]1Cc2cc(NC(=O)NC(C)(C)C)c(F)cc2N(Cc2ccccc2)C1=O ; C[C@@H]1Cc2cc(NC(=O)NC(C)(C)C)c(F)cc2N(Cc2ccccc2)C1=O]

### (S)-1-(1-benzyl-7-fluoro-3-methyl-2-oxo-1,2,3,4-tetrahydroquinolin-6-yl)-3-(tert-butyl)urea and (R)-1-(1-benzyl-7-fluoro-3-methyl-2-oxo-1,2,3,4-tetrahydroquinolin-6-yl)-3-(tert-butyl)urea

A sample of 1-(1-benzyl-7-fluoro-3-methyl-2-oxo-3,4-dihydroquinolin-6-yl)-3-tert-butylurea (70 mg, 0.183 mmol, 1 equiv) was separated by CHIRALPAK IG, 5*25 cm, 5 μm (eluent: 30% to 30% (v/v) Hex(0.1% 2M NH₃-MeOH)-HPLC and EtOH to yield 1-[(3S)-1-benzyl-7-fluoro-3-methyl-2-oxo-3,4-dihydroquinolin-6-yl]-3-tert-butylurea (29.3 mg, 41.21%) (first enantiomer to elute, labelled *S) MS (ESI): mass calcd. for C₂₂H₂₆FN₃O₂: 383.20 m/z, found 382.00 [M−H]⁻. ¹H NMR (300 MHz, DMSO-d₆) δ 7.92-7.95 (m, 2H), 7.27-7.38 (m, 2H), 7.15-7.27 (m, 3H), 6.75-6.79 (d, J=12 Hz, 1H), 6.43 (s, 1H), 5.10 (s, 2H), 2.91-2.95 (d, J=12 Hz, 1H), 2.59-2.74 (m, 2H), 1.27 (s, 9H), 1.15-1.19 (m, 3H). ¹⁹F NMR (282 MHz, DMSO) δ−131.68 and 1-[(3R)-1-benzyl-7-fluoro-3-methyl-2-oxo-3,4-dihydroquinolin-6-yl]-3-tert-butylurea (18.6 mg, 26.42%) (second enantiomer to elute, labelled *R) MS (ESI): mass calcd. for C₂₂H₂₆FN₃O₂: 383.20 m/z, found 382.00 [M−H]⁻. ¹H NMR (300 MHz, DMSO-d₆) δ 7.92-7.95 (m, 2H), 7.27-7.38 (m, 2H), 7.15-7.27 (m, 3H), 6.75-6.79 (d, J=12 Hz, 1H), 6.43 (s, 1H), 5.10 (s, 2H), 2.91-2.95 (m, 1H), 2.59-2.79 (m, 2H), 1.27 (s, 9H), 1.17-1.19 (d, J=6 Hz, 3H). ¹⁹F NMR (282 MHz, DMSO) δ −132.02.

### Example 335: 3-tert-butyl-1-[(2R)-4-{[3-(difluoromethoxy)phenyl]methyl}-6,8-difluoro-2-methyl-3-oxo-2H-1,4-benzoxazin-7-yl]urea

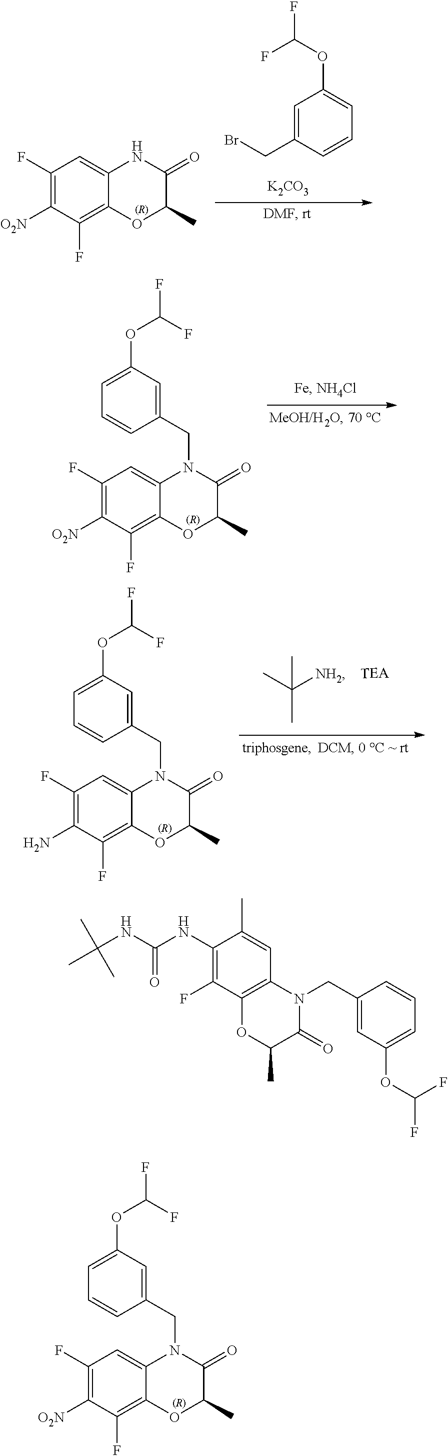

### (2R)-4-{[3-(difluoromethoxy)phenyl]methyl}-6,8-difluoro-2-methyl-7-nitro-2H-1,4-benzoxazin-3-one

To a stirred mixture of (2R)-6,8-difluoro-2-methyl-7-nitro-2,4-dihydro-1,4-benzoxazin-3-one (200 mg, 0.819 mmol, 1 equiv) and 1-(bromomethyl)-3-(difluoromethoxy)benzene (388.35 mg, 1.638 mmol, 2 equiv) in DMF (10 mL) was added K₂CO₃ (342.11 mg, 2.457 mmol, 3 equiv) in portions at room temperature under air atmosphere. The resulting mixture was stirred for 4 h at 80° C. under air atmosphere. After cooling down to room temperature, the reaction was quenched with water (30 mL) and extracted with EA (3×30 mL). The combined extracts were washed with water, brine, dried over Na₂SO₄, filtered and concentrated. TLC showed the desired product was generated. The residue was purified by column chromatography on silica gel with EA/PE (0-100%) to afford (2R)-4-{[3-(difluoromethoxy)phenyl]methyl}-6,8-difluoro-2-methyl-7-nitro-2H-1,4-benzoxazin-3-one (314 mg, 95.76%) as a white solid.

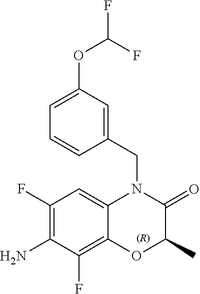

### (2R)-7-amino-4-{[3-(difluoromethoxy)phenyl]methyl}-6,8-difluoro-2-methyl-2H-1,4-benzoxazin-3-one

To a stirred mixture of (2R)-4-{[3-(difluoromethoxy)phenyl]methyl}-6,8-difluoro-2-methyl-7-nitro-2H-1,4-benzoxazin-3-one (300 mg, 0.749 mmol, 1 equiv) and NH₄C₁ (400.89 mg, 7.490 mmol, 10 equiv) in MeOH:H₂O=10:1 was added Fe (418.54 mg, 7.490 mmol, 10 equiv) in portions at room temperature under air atmosphere. The resulting mixture was stirred for 1 h at 80° C. under air atmosphere. After cooling down to room temperature, the reaction was quenched with water (20 mL) and extracted with EA (3×20 mL). The combined extracts were washed with water, brine, dried over Na₂SO₄, filtered and concentrated. The crude product was used in the next step directly without further purification. The residue afforded (2R)-7-amino-4-{[3-(difluoromethoxy)phenyl]methyl}-6,8-difluoro-2-methyl-2H-1,4-benzoxazin-3-one (207 mg, 74.59%) as a yellow oil.

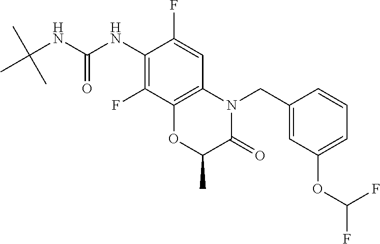 [SMILES: C[C@H]1Oc2c(cc(F)c(NC(=O)NC(C)(C)C)c2F)N(Cc2cccc(OC(F)F)c2)C1=O]

### 3-tert-butyl-1-[(2R)-4-{[3-(difluoromethoxy)phenyl]methyl}-6,8-difluoro-2-methyl-3-oxo-2H-1,4-benzoxazin-7-yl]urea

To a stirred solution of (2R)-7-amino-4-{[3-(difluoromethoxy)phenyl]methyl}-6,8-difluoro-2-methyl-2H-1,4-benzoxazin-3-one (120 mg, 0.324 mmol, 1 equiv) and triphosgene (57.69 mg, 0.194 mmol, 0.6 equiv) in DCM (5 mL) was added TEA (98.38 mg, 0.972 mmol, 3 equiv) dropwise at 0° C. under nitrogen atmosphere. The resulting mixture was stirred for 30 min at room temperature under nitrogen atmosphere. To the above mixture was added 2-methylpropan-2-amine (23.70 mg, 0.324 mmol, 1 equiv) dropwise at room temperature. The resulting mixture was stirred for additional 30 min at room temperature. The reaction was quenched with water (20 mL) and extracted with DCM (3×30 mL). The combined extracts were washed with water, brine, dried over Na₂SO₄, filtered and concentrated. The residue was purified by preparative HPLC using a YMC-Actus Triart Cis ExRS 150 mm×30 mm×5 μm column (eluent: 17% to 42% (v/v) CH₃CN and H₂O with 10 mmol/L NH₄HCO₃) to afford 3-tert-butyl-1-[(2R)-4-{[3-(difluoromethoxy)phenyl]methyl}-6,8-difluoro-2-methyl-3-oxo-2H-1,4-benzoxazin-7-yl]urea (46.4 mg, 30.46%) as a white solid. LC/MS (ESI): mass calcd. for C₂₂H₂₃F₄N₃O₄:469.1 m/z, found: 470.10 [M+H]⁺. ¹H NMR (400 MHz, DMSO-d₆) δ 7.51 (s, 1H), 7.01-7.47 (m, 5H), 6.80-7.00 (m, 1H), 6.15 (s, 1H), 5.06-5.27 (m, 2H), 4.90-5.06 (m, 1H), 1.45-1.61 (m, 3H), 1.09-1.35 (m, 9H). ¹⁹F NMR (376 MHz, DMSO) δ −81.89, −126.18 −139.24.

### Example 336: (R)-1-(tert-butyl)-3-(4-(3-(difluoromethyl)benzyl)-6,8-difluoro-2-methyl-3-oxo-3,4-dihydro-2H-benzo[b][1,4]oxazin-7-yl)urea

### Synthetic Scheme

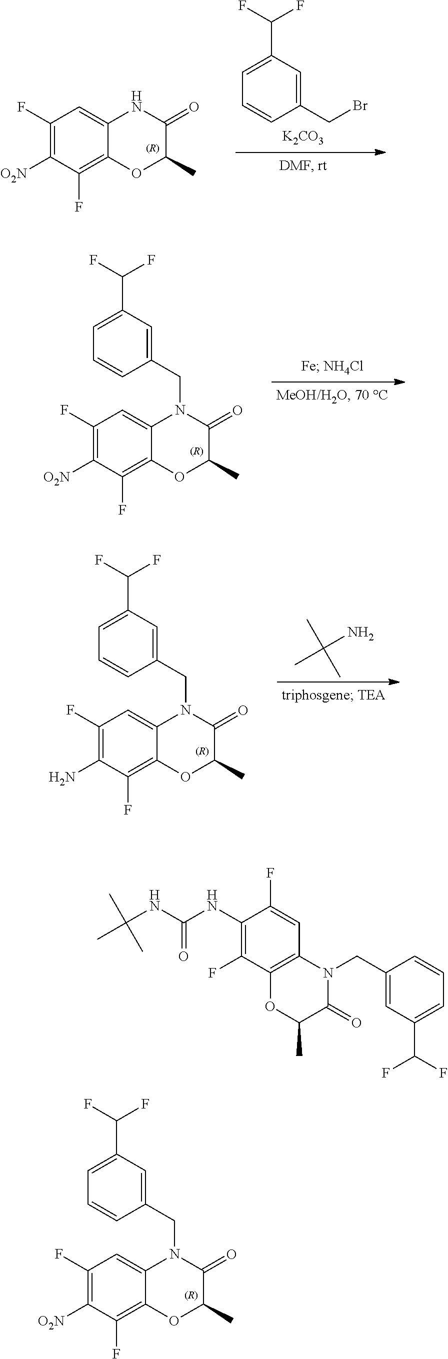

### (2R)-4-{[3-(difluoromethyl)phenyl]methyl}-6,8-difluoro-2-methyl-7-nitro-2H-1,4-benzoxazin-3-one

To a stirred solution of (2R)-6,8-difluoro-2-methyl-7-nitro-2,4-dihydro-1,4-benzoxazin-3-one (340 mg, 1.393 mmol, 1 equiv) and 1-(bromomethyl)-3-(difluoromethyl)benzene (338.60 mg, 1.532 mmol, 1.1 equiv) in DMF (10 mL) was added K₂CO₃ (581.59 mg, 4.179 mmol, 3 equiv) in portions at room temperature under air atmosphere. The resulting mixture was stirred for 1 h at room temperature under air atmosphere. The resulting mixture was extracted with EtOAc (3×20 mL). The combined organic layers were washed with brine (3×20 mL), dried over anhydrous MgSO₄. After filtration, the filtrate was concentrated under reduced pressure. The residue was purified by silica gel column chromatography, eluted with PE/EA (1:3) to afford (2R)-4-{[3-(difluoromethyl)phenyl]methyl}-6,8-difluoro-2-methyl-7-nitro-2H-1,4-benzoxazin-3-one (350 mg, 65.40%) as a yellow solid. TLC showed the desired product was generated.

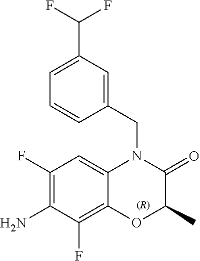

### (2R)-7-amino-4-{[3-(difluoromethyl)phenyl]methyl}-6,8-difluoro-2-methyl-2H-1,4-benzoxazin-3-one

To a stirred mixture of (2R)-4-{[3-(difluoromethyl)phenyl]methyl}-6,8-difluoro-2-methyl-7-nitro-2H-1,4-benzoxazin-3-one (350 mg, 0.911 mmol, 1 equiv) and Fe (508.62 mg, 9.110 mmol, 10 equiv) in MeOH/water (10:1, 5 mL) was added NH₄C₁ (487.17 mg, 9.110 mmol, 10 equiv) in portions at room temperature under air atmosphere. The resulting mixture was stirred for 1 h at 70° C. under air atmosphere. Desired product could be detected by LCMS. The resulting mixture was extracted with EtOAc (3×10 mL). The combined organic layers were washed with water (3×10 mL), dried over anhydrous MgSO₄. After filtration, the filtrate was concentrated under reduced pressure. The residue was purified by silica gel column chromatography, eluted with PE/EA (1:1) to afford (2R)-7-amino-4-{[3-(difluoromethyl)phenyl]methyl}-6,8-difluoro-2-methyl-2H-1,4-benzoxazin-3-one (280 mg, 86.77%) as a yellow solid. LC/MS (ESI): mass calcd. for C₁₇H₁₄F₄N₂O₂:354.10 m/z, found: 354.95 [M+H]⁺.

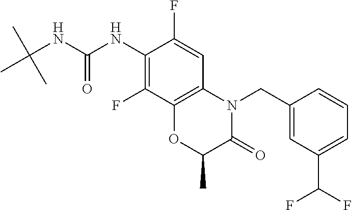 [SMILES: C[C@H]1Oc2c(cc(F)c(NC(=O)NC(C)(C)C)c2F)N(Cc2cccc(C(F)F)c2)C1=O]

### 3-tert-butyl-1-[(2R)-4-{[3-(difluoromethyl)phenyl]methyl}-6,8-difluoro-2-methyl-3-oxo-2H-1,4-benzoxazin-7-yl]urea

A solution of (2R)-7-amino-4-{[3-(difluoromethyl)phenyl]methyl}-6,8-difluoro-2-methyl-2H-1,4-benzoxazin-3-one (60 mg, 0.169 mmol, 1 equiv) in DCM (5 mL) was treated with triphosgene (100.50 mg, 0.338 mmol, 2 equiv) for 1 h at 0 C under nitrogen atmosphere followed by the addition of triethylamine (34.27 mg, 0.338 mmol, 2 equiv) in portions at 0° C. To the above mixture was added erbumine (24.77 mg, 0.338 mmol, 2 equiv) and triethylamine (34.27 mg, 0.338 mmol, 2 equiv) in portions at 0° C. The resulting mixture was stirred for additional 1 h at room temperature. Desired product could be detected by LCMS. The resulting mixture was extracted with EtOAc (3×10 mL). The combined organic layers were washed with water (3×10 mL), dried over anhydrous MgSO₄. After filtration, the filtrate was concentrated under reduced pressure. The residue was purified by preparative HPLC using a XBridge Prep OBD 250 mm×19 mm×5 μm column (eluent: 42% to 72% (v/v) ACN and Water with 10 mmol/L NH₄HCO₃) to afford 3-tert-butyl-1-[(2R)-4-{[3-(difluoromethyl)phenyl]methyl}-6,8-difluoro-2-methyl-3-oxo-2H-1,4-benzoxazin-7-yl]urea (3.6 mg, 4.55%) as a white solid. LC/MS (ESI): mass calcd. for C₂₂H₂₃F₄N₃O₃:453.17 m/z, found: 452.00 [M−H]⁻. ¹H NMR (400 MHz, Acetonitrile-d₃) δ 7.45-7.60 (m, 4H), 6.60-7.00 (m, 2H), 6.10-6.30 (s, 1H), 5.20-5.40 (s, 1H), 5.10-5.20 (m, 2H), 4.80-4.95 (m, 1H), 1.55-1.65 (m, 3H), 1.28-1.38 (s, 9H). ¹⁹F NMR (376 MHz, Acetonitrile-d3) δ −111.38, −128.07, −141.04.

### Example 337: 1-(1-methylcyclobutyl)-3-(2-oxo-1-(1-(4-(trifluoromethyl)pyridin-2-yl)ethyl)-1,2-dihydroquinoxalin-6-yl)urea

### Synthetic Scheme

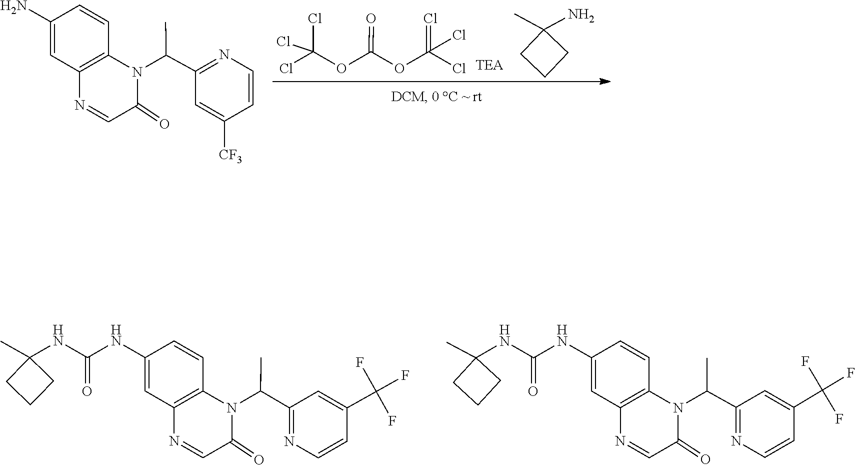

### 1-(1-methylcyclobutyl)-3-(2-oxo-1-(1-(4-(trifluoromethyl)pyridin-2-yl)ethyl)-1,2-dihydroquinoxalin-6-yl)urea

To a solution of 6-amino-1-{1-[4-(trifluoromethyl)pyridin-2-yl]ethyl}quinoxalin-2-one (70 mg, 0.209 mmol, 1 equiv) in DCM (5 mL) was added triphosgene (62.13 mg, 0.209 mmol, 1 equiv) under N₂ at 0° C. The reaction was stirred at 0° C. under N₂ for 1 h. To the above mixture was added TEA (63.57 mg, 0.627 mmol, 3 equiv) at 0° C. The resulting mixture was stirred for additional 1 h at rt. Then, to the above mixture was added 1-methylcyclobutan-1-amine (53.49 mg, 0.627 mmol, 3 equiv). The resulting mixture was stirred for additional 1 h at rt. Quenched with water (10 mL) and extracted with DCM (3×10 mL). The combined extracts were washed with water, brine, dried over Na₂SO₄, filtered and concentrated. The residue was purified by preparative HPLC using a XBridge Prep OBD C18 Column30*150 mm 5 μm column (eluent: 33% to 47% (v/v) CH₃CN and Water (10 mmol/L NH₄HCO₃+0.1% NH₃·H₂O)) to afford the title compound 3-(1-methylcyclobutyl)-1-(2-oxo-1-{1-[4-(trifluoromethyl)pyridin-2-yl]ethyl}quinoxalin-6-yl)urea (43 mg, 46.05%) as a yellow solid. LC/MS: mass calcd. for C₂₁H₂₂F₃N₅o₂:445.17 m/z, found: 446.05 [M+H]⁺. ¹H NMR (400 MHz, DMSO-d₆) δ 8.78 (d, J=5.1 Hz, 1H), 8.46 (s, 1H), 8.20 (s, 1H), 7.96 (d, J=2.5 Hz, 1H), 7.75 (s, 1H), 7.69 (d, J=5.2 Hz, 1H), 7.10-7.56 (m, 2H), 6.54 (s, 1H), 6.33 (s, 1H), 2.29 (dt, J=11.4, 9.1 Hz, 2H), 1.84-1.97 (m, 5H), 1.70-1.83 (m, 2H), 1.40 (s, 3H). ¹⁹F NMR (376 MHz, DMSO) δ(ppm): −663.0913.

### Example 338: (R)-1-(tert-butyl)-3-(4-(3-(difluoromethoxy)benzyl)-6-fluoro-2-methyl-3-oxo-3,4-dihydro-2H-benzo[b][1,4]oxazin-7-yl)urea

### Synthetic Scheme

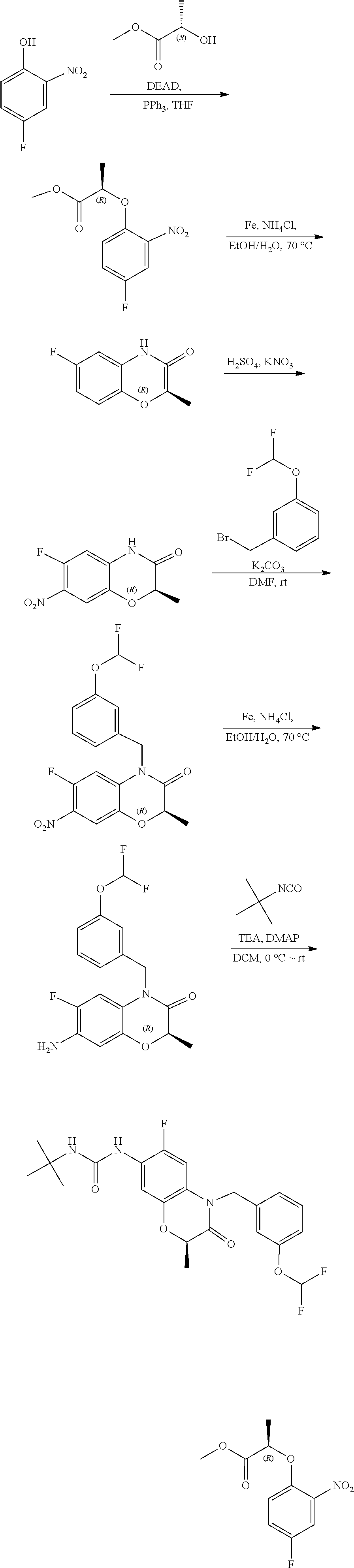

### methyl (R)-2-(4-fluoro-2-nitrophenoxy)propanoate

A solution of 4-fluoro-2-nitrophenol (2 g, 12.731 mmol, 1 equiv) in THF (20 mL) was treated with methyl (2S)-2-hydroxypropanoate (1.99 g, 19.096 mmol, 1.5 equiv) and PPh₃ (5.01 g, 19.096 mmol, 1.5 equiv) for 1 h at rt under nitrogen atmosphere followed by the addition of DEAD (3.33 g, 19.096 mmol, 1.5 equiv) dropwise at 0° C. The resulting mixture was stirred for 2 h at 60° C. under N₂ atmosphere. The mixture was allowed to cool down to rt. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give methyl (2R)-2-(4-fluoro-2-nitrophenoxy)propanoate (1 g, 32.30%) as a white solid. MS (ESI): mass calcd. for C₁₀H₁₀FNO₅: 243.05 m/z, found 244.15 [M+H]⁺.

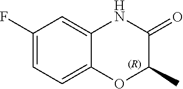

### (R)-6-fluoro-2-methyl-2H-benzo[b][1,4]oxazin-3(4H)-one

To a solution of methyl (2R)-2-(4-fluoro-2-nitrophenoxy)propanoate (1 g, 4.112 mmol, 1 equiv) in EtOH (10 mL)/H₂O (1 mL) was added Fe (2.30 g, 41.120 mmol, 10 equiv) and NH₄C₁ (2.20 g, 41.120 mmol, 10 equiv). The reaction was stirred at 70° C. for 3 h. The mixture was allowed to cool down to rt. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-6-fluoro-2-methyl-2,4-dihydro-1,4-benzoxazin-3-one (650 mg, 87.25%) as a white solid. MS (ESI): mass calcd. for C₉H₈FNO₂: 181.05 m/z, found 182.05 [M+H]⁺.

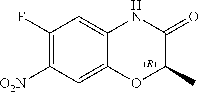

### (R)-6-fluoro-2-methyl-7-nitro-2H-benzo[b][1,4]oxazin-3(4H)-one

A solution of (2R)-6-fluoro-2-methyl-2,4-dihydro-1,4-benzoxazin-3-one (1 g, 5.520 mmol, 1 equiv) and KNO₃ (558.06 mg, 5.520 mmol, 1 equiv) in H₂SO₄ (10 mL) was stirred for 1 h at 0° C. The reaction was stirred at rt for 1 h. Quenched with ice water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-6-fluoro-2-methyl-7-nitro-2,4-dihydro-1,4-benzoxazin-3-one (660 mg, 52.87%) as a white solid. MS (ESI): mass calcd. for C₉H₇FN₂O₄: 226.04 m/z, found 224.95 [M−H]⁻.

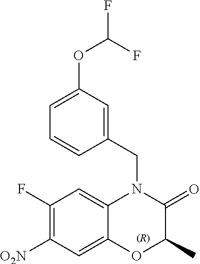

### (R)-4-(3-(difluoromethoxy)benzyl)-6-fluoro-2-methyl-7-nitro-2H-benzo[b][1,4]oxazin-3(4H)-one

To a solution of (2R)-6-fluoro-2-methyl-7-nitro-2,4-dihydro-1,4-benzoxazin-3-one (300 mg, 1.326 mmol, 1 equiv) in DMF (5 mL) was added 1-(bromomethyl)-3-(difluoromethoxy)benzene (377.32 mg, 1.591 mmol, 1.2 equiv) and K₂CO₃ (553.99 mg, 3.978 mmol, 3 equiv). The reaction was stirred at rt for 3 h. Quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give (2R)-4-{[3-(difluoromethoxy)phenyl]methyl}-6-fluoro-2-methyl-7-nitro-2H-1,4-benzoxazin-3-one (450 mg, 88.74%) as a white solid. MS (ESI): mass calcd. for C₁₇H₁₃F₃N₂O₅: 382.08 m/z, found 383.10 [M+H]⁺.

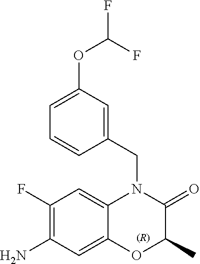

### (R)-7-amino-4-(3-(difluoromethoxy)benzyl)-6-fluoro-2-methyl-2H-benzo[b][1,4]oxazin-3(4H)-one

To a solution of (2R)-4-{[3-(difluoromethoxy)phenyl]methyl}-6-fluoro-2-methyl-7-nitro-2H-1,4-benzoxazin-3-one (514 mg, 1.345 mmol, 1 equiv) in EtOH (10 mL) was added Fe (750.84 mg, 13.450 mmol, 10 equiv) and NH₄C₁ (719.18 mg, 13.450 mmol, 10 equiv). The reaction was stirred at 70° C. for 3 h. The mixture was allowed to cool down to rt. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-7-amino-4-{[3-(difluoromethoxy)phenyl]methyl}-6-fluoro-2-methyl-2H-1,4-benzoxazin-3-one (450 mg, 95.00%) as a white solid. MS (ESI): mass calcd. for C₁₇H₁₅F₃N₂O₃: 352.10 m/z, found 353.05 [M+H]⁺.

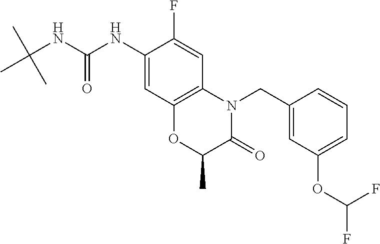 [SMILES: C[C@H]1Oc2cc(NC(=O)NC(C)(C)C)c(F)cc2N(Cc2cccc(OC(F)F)c2)C1=O]

### (R)-1-(tert-butyl)-3-(4-(3-(difluoromethoxy)benzyl)-6-fluoro-2-methyl-3-oxo-3,4-dihydro-2H-benzo[b][1,4]oxazin-7-yl)urea

To a solution of (2R)-7-amino-4-{[3-(difluoromethoxy)phenyl]methyl}-6-fluoro-2-methyl-2H-1,4-benzoxazin-3-one (200 mg, 0.568 mmol, 1 equiv) in DCM (5 mL) was added 2-isocyanato-2-methylpropane (168.74 mg, 2.840 mmol, 5 equiv) and TEA (287.22 mg, 2.840 mmol, 5 equiv). The reaction was stirred at rt for 24 h. Quenched with water (10 mL) and extracted with DCM (3×10 mL). The combined extracts were washed with water, brine, dried over Na₂SO₄, filtered and concentrated. The residue was purified by preparative HPLC using a YMC-Actus Triart C18 ExRS 150 mm×30 mm×5 μm column (eluent: 49% to 73% (v/v) CH₃CN and H₂O with 10 mmol/L NH₄HCO₃) to afford the title compound 3-tert-butyl-1-[(2R)-4-{[3-(difluoromethoxy)phenyl]methyl}-6-fluoro-2-methyl-3-oxo-2H-1,4-benzoxazin-7-yl]urea (55.4 mg, 21.60%) as a off-white solid. LC/MS (ESI): mass calcd. for C₂₂H₂₄F₃N₃O₄: 451.17 m/z, found: 452.05 [M+H]⁺. ¹H NMR (300 MHz, DMSO-d₆) δ 8.07 (s, 1H), 7.82-7.84 (d, J=6 Hz, 1H), 6.95-7.46 (m, 6H), 6.51 (s, 1H), 5.12 (s, 2H), 4.78-4.85 (m, 1H), 1.46-1.48 (d, J=6.0 Hz, 3H), 1.27 (s, 9H). ¹⁹F NMR (282 MHz, DMSO) δ−81.91−137.08.

### Example 339: (S)-1-(tert-butyl)-3-(7-fluoro-2-oxo-1-(1-(3-(trifluoromethoxy)phenyl)ethyl)-1,2-dihydroquinoxalin-6-yl)urea

### Synthetic Scheme

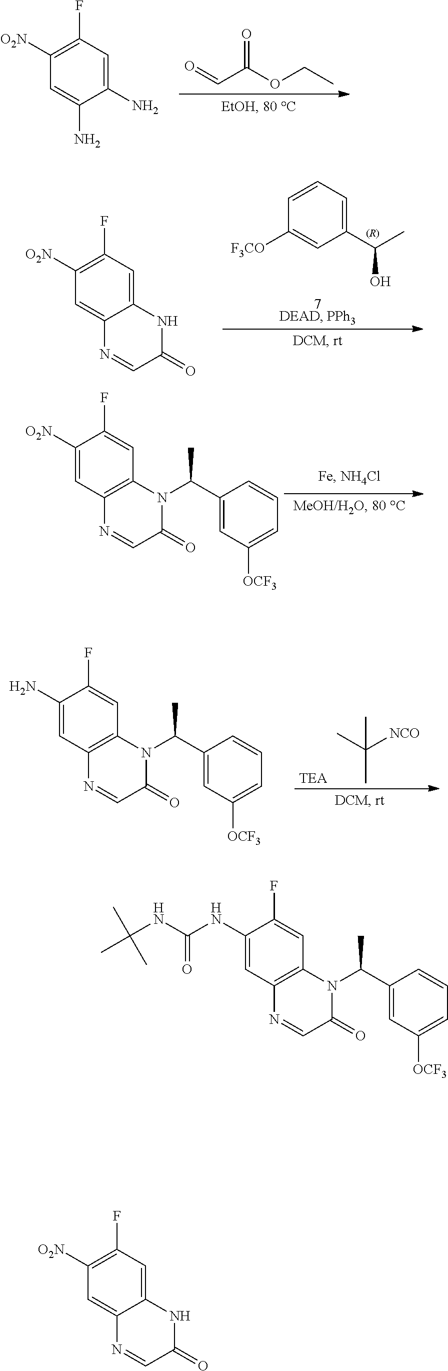

### 7-fluoro-6-nitro-1H-quinoxalin-2-one

To a solution of 4-fluoro-5-nitrobenzene-1,2-diamine (2 g, 11.687 mmol, 1 equiv) in ethyl alcohol (10 mL) was added ethyl glyoxylate (1.19 g, 11.687 mmol, 1 equiv). The resulting mixture was stirred for 2 h at 80° C. The reaction mixture was quenched by water (10 mL) and extracted with DCM (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 7-fluoro-6-nitroquinoxalin-2(1H)-one (1.7 g, 69.55%) as a white solid. MS (ESI): mass calcd. for C₈H₄FN₃O₃: 209.17 m/z, found: 208 [M−H]⁻.

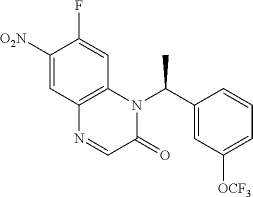 [SMILES: C[C@@H](c1cccc(OC(F)(F)F)c1)n1c(=O)cnc2cc([N+](=O)[O-])c(F)cc21]

### (S)-7-fluoro-6-nitro-1-(1-(3-(trifluoromethoxy)phenyl)ethyl)quinoxalin-2(1H)-one

To a solution of 7-fluoro-6-nitroquinoxalin-2(1H)-one (1.6 g, 7.651 mmol, 1.2 equiv) in DCM (40 mL) was added (1R)-1-[3-(trifluoromethoxy)phenyl]ethanol (1.31 g, 6.376 mmol, 1 equiv), PPh₃ (2.51 g, 9.564 mmol, 1.5 equiv) and DEAD (1.39 g, 7.970 mmol, 1.25 equiv). The resulting mixture was stirred for 2 h at 25° C. under N₂ and stirred until the starting material was totally consumed by TLC. The reaction mixture was quenched by water (10 mL) and extracted with DCM (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford (S)-7-fluoro-6-nitro-1-(1-(3-(trifluoromethoxy)phenyl)ethyl)quinoxalin-2(1H)-one (2 g, 78.96%) as a white solid. MS (ESI): mass calcd. for C₁₇H₁₁F₄N₃O₄: 397.17 m/z, found: 398.05 [M+H]⁺.

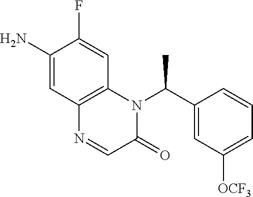 [SMILES: C[C@@H](c1cccc(OC(F)(F)F)c1)n1c(=O)cnc2cc(N)c(F)cc21]

### (S)-6-amino-7-fluoro-1-(1-(3-(trifluoromethoxy)phenyl)ethyl)quinoxalin-2(1H)-one

To a solution of (S)-7-fluoro-6-nitro-1-(1-(3-(trifluoromethoxy)phenyl)ethyl)quinoxalin-2(1H)-one (2 g, 5.034 mmol, 1 equiv) in EtOH (20 mL) and H₂O (2 mL) was added Fe (2.81 g, 50.340 mmol, 10 equiv) and NH₄C₁ (2.69 g, 50.340 mmol, 10 equiv). The resulting mixture was stirred for 2 h at 80° C. and stirred until the starting material was totally consumed by TLC. The reaction mixture was quenched by water and extracted with DCM (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford (S)-6-amino-7-fluoro-1-(1-(3-(trifluoromethoxy)phenyl)ethyl)quinoxalin-2(1H)-one (350 mg, 87.28%) as a white solid. MS (ESI): mass calcd. for C₁₇H₁₃F₄N₃O₂: 367.17 m/z, found: 368.15 [M+H]⁺.

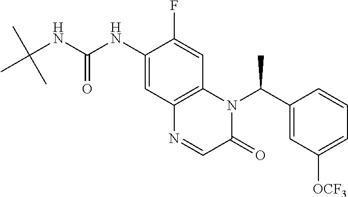 [SMILES: C[C@@H](c1cccc(OC(F)(F)F)c1)n1c(=O)cnc2cc(NC(=O)NC(C)(C)C)c(F)cc21]

### (S)-1-(tert-butyl)-3-(7-fluoro-2-oxo-1-(1-(3-(trifluoromethoxy)phenyl)ethyl)-1,2-dihydroquinoxalin-6-yl)urea

To a solution of (S)-6-amino-7-fluoro-1-(1-(3-(trifluoromethoxy)phenyl)ethyl)quinoxalin-2(1H)-one (80 mg, 0.218 mmol, 1 equiv) in DCM (20 mL) was added 2-isocyanato-2-methylpropane (64.77 mg, 0.654 mmol, 3 equiv), DMAP (2.66 mg, 0.022 mmol, 0.1 equiv) and TEA (66.12 mg, 0.654 mmol, 3 equiv). The resulting mixture was stirred for 24 h at rt. The reaction mixture was quenched by water and extracted with DCM (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 53% B in 9 min, 53% B; Wave Length: 254/220 nm; RT1(min): 8.52; Number Of Runs: 0) to afford (S)-1-(tert-butyl)-3-(7-fluoro-2-oxo-1-(1-(3-(trifluoromethoxy)phenyl)ethyl)-1,2-dihydroquinoxalin-6-yl)urea (26 mg, 25.52%) as an white solid. MS (ESI): mass calcd. For C₂₂H₂₂F₄N₄O₃: 466.17 m/z, found: 467.20 [M+H]⁺. ¹H NMR (400 MHz, DMSO-d₆) δ 8.58 (d, J=8.7 Hz, 1H), 8.23-7.27 (m, 2H), 7.44-7.50 (m, 1H), 7.30-7.35 (m, 3H), 7.13-7.17 (m, 1H), 6.58 (s, 1H), 6.46 (br, 1H), 1.88 (d, J=7.0 Hz, 3H), 1.30 (s, 9H).

### Example 340: (R)-1-(tert-butyl)-3-(4-(3-(difluoromethyl)benzyl)-5,6-difluoro-2-methyl-3-oxo-3,4-dihydro-2H-benzo[b][1,4]oxazin-7-yl)urea

### Synthetic Scheme

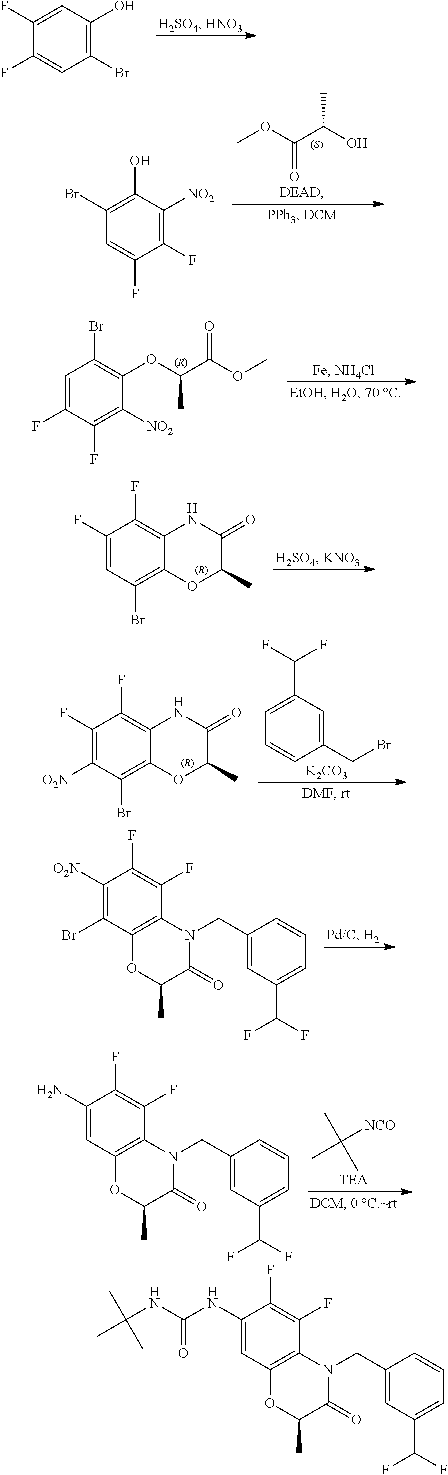

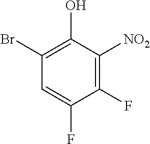 [SMILES: O=[N+]([O-])c1c(O)c(Br)cc(F)c1F]

### 6-bromo-3,4-difluoro-2-nitrophenol

To a solution of 2-bromo-4,5-difluorophenol (1.5 g, 7.177 mmol, 1 equiv) and HNO₃ (0.45 g, 7.177 mmol, 1 equiv) in H₂SO₄ (15 mL) was stirred for 1 h at 0° C. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (3:1) to afford 6-bromo-3,4-difluoro-2-nitrophenol (1.78 g, 83.00%) as a brown solid. LC/MS: mass calcd. for C₆H₂BrF₂NO₃: 252.92 m/z found: 253.92, 255.92 [M+H, M+H+2]⁺.

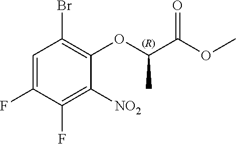

### methyl (R)-2-(6-bromo-3,4-difluoro-2-nitrophenoxy)propanoate

To a solution of methyl (2S)-2-hydroxypropanoate (0.73 g, 7.008 mmol, 1 equiv) in DCM (20 mL) was added 6-bromo-3,4-difluoro-2-nitrophenol (1.78 g, 7.008 mmol, 1 equiv) and PPh₃ (2.76 g, 10.512 mmol, 1.5 equiv). The resulting mixture was stirred for 1 h at rt under N₂. DEAD (1.43 g, 8.211 mmol, 1 equiv) was added the mixture at 0° C. under nitrogen atmosphere. The resulting mixture was stirred for 16 h at rt. The reaction mixture was quenched by water and extracted with EA (3×50 mL). The combined organic extracts were washed with brine (50 mL), dried over anhydrous Na₂SO₄, and concentrated under vacuum to yield a crude product which was directly purified by silica gel column, eluted with PE/EA (1:1) to afford methyl (R)-2-(6-bromo-3,4-difluoro-2-nitrophenoxy) propanoate (2 g, 71.33%) as yellow oil. LC/MS: mass calcd. for C₁₀H₈BrF₂NO₅: 338.96 m/z found: 339.96, 341.96 [M+H, M+H+2]⁺

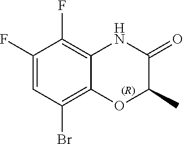

### (R)-8-bromo-5,6-difluoro-2-methyl-2H-benzo[b][1,4]oxazin-3(4H)-one

To a mixture of methyl (2R)-2-(6-bromo-3,4-difluoro-2-nitrophenoxy) propanoate (2 g, 5.881 mmol, 1 equiv) in EtOH (10 mL) and H₂O (2.5 mL) was added Fe (3.28 g, 58.810 mmol, 10 equiv) and NH₄C₁ (3.15 g, 58.810 mmol, 10 equiv). The resulting mixture was stirred for 2 h at 70° C. The resulting mixture was filtered, the filter cake was washed with EtOH. The filtrate was concentrated under vacuum to yield a crude product which was directly purified by silica gel column, eluted with PE/EA (2:1) to afford (2R)-8-bromo-5,6-difluoro-2-methyl-2,4-dihydro-1,4-benzoxazin-3-one (1.35 g, 82.56%) as a yellow semi-solid. LC/MS: mass calcd. for C₉H₆BrF₂NO₂: 276.95 m/z found: 277.95, 279.95 [M+H, M+H+2]⁺

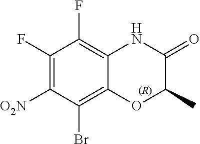

### (R)-8-bromo-5,6-difluoro-2-methyl-7-nitro-2H-benzo[b][1,4]oxazin-3(4H)-one

To a solution of (2R)-8-bromo-5,6-difluoro-2-methyl-2,4-dihydro-1,4-benzoxazin-3-one (1000 mg, 3.60 mmol, 1 equiv) and KNO₃ (727.23 mg, 7.20 mmol, 2 equiv) in H₂SO₄ (20 mL) was stirred for 12 h at rt. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (3:1) to afford (2R)-8-bromo-5,6-difluoro-2-methyl-2,4-dihydro-1,4-benzoxazin-3-one (1.35 g, 82.56%) as a yellow semi-solid. LC/MS: mass calcd. for C₉H₅BrF₂N₂O₄: 321.94 m/z found: 322.94, 324.94 [M+H, M+H+2]⁺

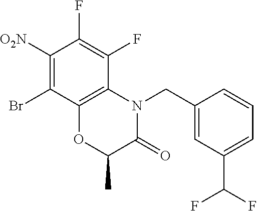 [SMILES: C[C@H]1Oc2c(Br)c([N+](=O)[O-])c(F)c(F)c2N(Cc2cccc(C(F)F)c2)C1=O]

### (R)-8-bromo-4-(3-(difluoromethyl)benzyl)-5,6-difluoro-2-methyl-7-nitro-2H-benzo[b][1,4]oxazin-3(4H)-one

To a solution of (2R)-8-bromo-5,6-difluoro-2-methyl-7-nitro-2,4-dihydro-1,4-benzoxazin-3-one (390 mg, 1.207 mmol, 1 equiv) in DMF (5 mL) was added 1-(bromomethyl)-3-(difluoromethyl) benzene (266.86 mg, 1.207 mmol, 1 equiv) and K₂CO₃ (333.69 mg, 2.414 mmol, 2 equiv). The resulting mixture was stirred for 16 h at rt. The reaction mixture was quenched by water and extracted with EA (3×50 mL). The combined organic extracts were washed with brine (20 mL), dried over anhydrous Na₂SO₄, and concentrated under vacuum to yield a crude product which was directly purified by silica gel column, eluted with PE/EA (2:1) to afford (2R)-8-bromo-4-{[3-(difluoromethyl)phenyl]methyl}-5,6-difluoro-2-methyl-7-nitro-2H-1,4-benzoxazin-3-one (260 mg, 39.52%) as yellow oil. LC/MS: mass calcd. for C₁₇H₁₁BrF₄N₂O₄: 461.98 m/z found: 462.98, 464.98 [M+H, M+H+2]⁺.

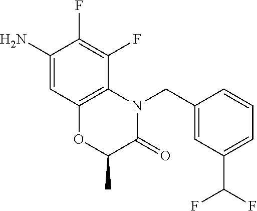 [SMILES: C[C@H]1Oc2cc(N)c(F)c(F)c2N(Cc2cccc(C(F)F)c2)C1=O]

### (R)-7-amino-4-(3-(difluoromethyl)benzyl)-5,6-difluoro-2-methyl-2H-benzo[b][1,4]oxazin-3(4H)-one

A mixture of (2R)-8-bromo-4-{[3-(difluoromethyl)phenyl]methyl}-5,6-difluoro-2-methyl-7-nitro-2H-1,4-benzoxazin-3-one (250 mg, 0.540 mmol, 1 equiv) and Pd/C (574.40 mg, 5.400 mmol, 10 equiv) in MeOH (5 mL) was stirred for 1 h at rt under H₂ atmosphere. The resulting mixture was filtered, the filter cake was washed with MeOH (20 mL). The filtrate was concentrated under vacuum to yield a crude product which was directly purified by silica gel column, eluted with PE/EA (2:1) to afford (2R)-7-amino-4-{[3-(difluoromethyl) phenyl]methyl}-5,6-difluoro-2-methyl-2H-1,4-benzoxazin-3-one (100 mg, 44.45%) as a yellow solid. LC/MS: mass calcd. for C₁₇H₁₄F₄N₂O₂: 354.10 m/z found: 355.10 [M+H]⁺.

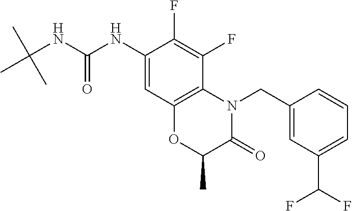 [SMILES: C[C@H]1Oc2cc(NC(=O)NC(C)(C)C)c(F)c(F)c2N(Cc2cccc(C(F)F)c2)C1=O]

### (R)-1-(tert-butyl)-3-(4-(3-(difluoromethyl) benzyl)-5,6-difluoro-2-methyl-3-oxo-3,4-dihydro-2H-benzo[b][1,4]oxazin-7-yl)urea

To a solution of (2R)-7-amino-4-{[3-(difluoromethyl)phenyl]methyl}-5,6-difluoro-2-methyl-2H-1,4-benzoxazin-3-one (90 mg, 0.254 mmol, 1 equiv) in DCM (5 mL) was added TEA (77.11 mg, 0.762 mmol, 3 equiv) and 2-isocyanato-2-methylpropane (76.2 mg, 0.762 mmol, 3 equiv). The resulting mixture was stirred for 16 h at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 53% B in 9 min, 53% B; Wave Length: 254/220 nm; RT1(min): 8.52; Number Of Runs: 0) to afford 3-tert-butyl-1-[(2R)-4-{[3-(difluoromethyl)phenyl]methyl}-5,6-difluoro-2-methyl-3-oxo-2H-1,4-benzoxazin-7-yl]urea (4.7 mg, 4.02%) as a white solid. LC/MS: mass calcd. for C₂₂H₂₃F₄N₃O₃: 453.17 m/z found: 452.10 [M−H]⁻. ¹H NMR (300 MHz, Chloroform-d) δ 7.68 (dd, J=7.0, 2.3 Hz, 1H), 7.34-7.44 (m, 2H), 7.31 (s, 1H), 6.62 (s, 1H), 6.26 (s, 1H), 5.35 (d, J=15.6 Hz, 1H), 5.18 (d, J=15.5 Hz, 1H), 4.65 (s, 1H), 4.60 (q, J=6.7 Hz, 2H), 1.59 (d, J=6.8 Hz, 3H), 1.40 (s, 9H). ¹⁹F-NMR (282 MHz, CDCl₃) δ −110.63, −147.41, −161.57.

### Example 341: 1-(4-benzyl-6-cyano-3-oxo-3,4-dihydro-2H-benzo[b][1,4]oxazin-7-yl)-3-(tert-butyl)urea

### Synthetic Scheme

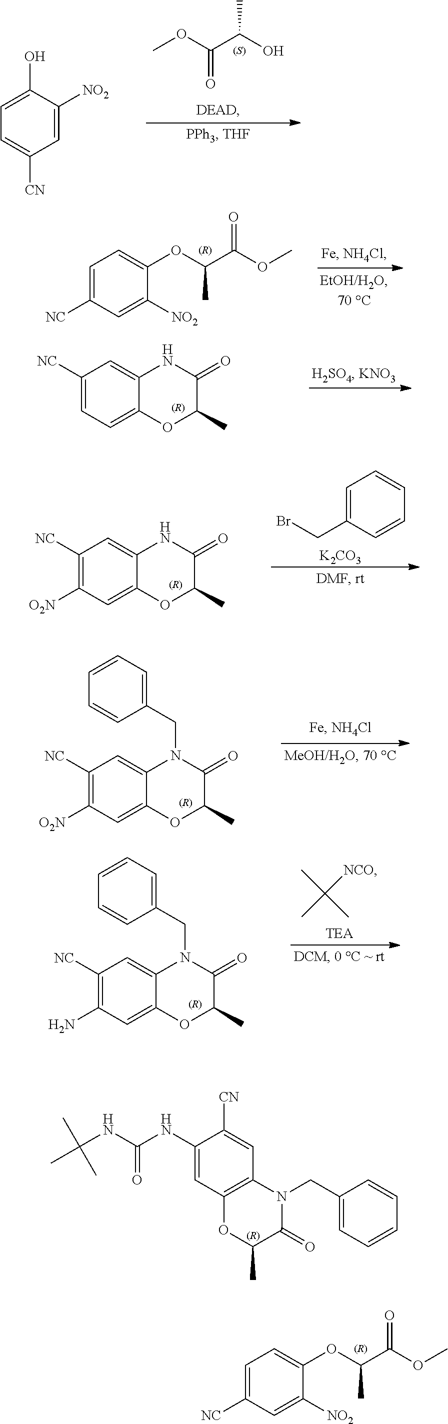

### methyl (R)-2-(4-cyano-2-nitrophenoxy) propanoate

To a stirred solution of 4-hydroxy-3-nitrobenzonitrile (2.5 g, 15.233 mmol, 1 equiv) and methyl (2S)-2-hydroxypropanoate (1.59 g, 15.233 mmol, 1 equiv) in THF (20 mL) was added PPh₃ (5.99 g, 22.849 mmol, 1.5 equiv) in portions at 0° C. under nitrogen atmosphere. The resulting mixture was stirred for 0.5 h at room temperature under nitrogen atmosphere. Then, to the above solution was added DEAD (3.98 g, 22.849 mmol, 1.5 equiv) in portions at 0° C. under nitrogen atmosphere. The resulting mixture was stirred for 2 h at room temperature under nitrogen atmosphere. Desired product could be detected by LCMS. The reaction was quenched with water (100 mL). The resulting mixture was extracted with ethyl acetate (3×100 mL). The organic layers were combined, dried over anhydrous sodium sulfate, filtered and concentrated. The residue was purified by silica gel column chromatography, eluted with PE/EA (1:1) to afford methyl (R)-2-(4-cyano-2-nitrophenoxy) propanoate (3 g, 74.78%) as a yellow green oil. LC/MS: mass calcd for C₁₁H₁₀N₂O₅:250.06, found: 251.05 [M+H]⁺.

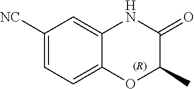

### (R)-2-methyl-3-oxo-3,4-dihydro-2H-benzo[b][1,4]oxazine-6-carbonitrile

To a stirred solution of methyl (R)-2-(4-cyano-2-nitrophenoxy) propanoate (3 g, 11.990 mmol, 1 equiv) and NH₄C₁ (6413.41 mg, 119.900 mmol, 10 equiv) in EtOH/H₂O (4:1, 40 mL) was added Fe (6695.78 mg, 119.900 mmol, 10 equiv) in portions at room temperature under air atmosphere. The resulting mixture was stirred for 2 h at 70° C. under air atmosphere. Desired product could be detected by LCMS. After cooling down to rt, the reaction was quenched with water (100 mL). The resulting mixture was extracted with ethyl acetate (3×100 mL). The organic layers were combined, dried over anhydrous sodium sulfate, filtered and concentrated. The residue was purified by silica gel column chromatography, eluted with PE/EA (1:1) to afford (R)-2-methyl-3-oxo-3,4-dihydro-2H-benzo[b][1,4]oxazine-6-carbonitrile (1.6 g, 49.64%) as a yellow oil. LC/MS: mass calcd for C₁₀H₈N₂O₂; 188.06, found: 189.05 [M+H]⁺.

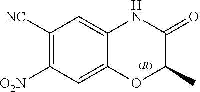

### (R)-2-methyl-7-nitro-3-oxo-3,4-dihydro-2H-benzo[b][1,4]oxazine-6-carbonitrile

To a stirred solution of (R)-2-methyl-3-oxo-3,4-dihydro-2H-benzo[b][1,4]oxazine-6-carbonitrile (1.6 g, 8.502 mmol, 1 equiv) in H₂SO₄ (20 mL, 375.242 mmol, 44.13 equiv) was added KNO₃ (859.59 mg, 8.502 mmol, 1 equiv) in portions at 0° C. under air atmosphere. The resulting mixture was stirred for 2 h at room temperature under air atmosphere. Desired product could be detected by LCMS. Pour the reaction into ice/water. The resulting mixture was extracted with ethyl acetate (3×30 mL). The organic layers were combined, dried over anhydrous sodium sulfate, filtered and concentrated. The residue was purified by silica gel column chromatography, eluted with PE/EA (1:1) to afford (R)-2-methyl-7-nitro-3-oxo-3,4-dihydro-2H-benzo[b][1,4]oxazine-6-carbonitrile (313 mg, 15.00%) as a yellow oil. LC/MS: mass calcd for C₁₀H₇N₃O₄: 233.04, found: 234.05 [M+H]⁺.

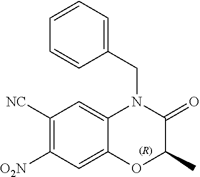

### (R)-4-benzyl-2-methyl-7-nitro-3-oxo-3,4-dihydro-2H-benzo[b][1,4]oxazine-6-carbonitrile

To a stirred solution of (R)-2-methyl-7-nitro-3-oxo-3,4-dihydro-2H-benzo[b][1,4]oxazine-6-carbonitrile (150 mg, 0.643 mmol, 1 equiv) and benzyl bromide (165.03 mg, 0.965 mmol, 1.5 equiv) in DMF (5 mL) was added K₂CO₃ (268.66 mg, 1.929 mmol, 3 equiv) in portions at room temperature under air atmosphere. The resulting mixture was stirred for 1 h at room temperature under air atmosphere. Desired product could be detected by LCMS. The reaction was quenched with water (10 mL). The resulting mixture was extracted with ethyl acetate (3×10 mL). The organic layers were combined, dried over anhydrous sodium sulfate, filtered and concentrated. The residue was purified by silica gel column chromatography, eluted with PE/EA (1:1) to afford (R)-4-benzyl-2-methyl-7-nitro-3-oxo-3,4-dihydro-2H-benzo[b][1,4]oxazine-6-carbonitrile (124 mg, 56.64%) as a yellow solid. LC/MS: mass calcd for C₁₇H₁₃N₃O₄: 323.09, found: 324.01 [M+H]⁺.

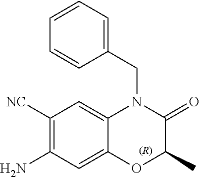

### (R)-7-amino-4-benzyl-2-methyl-3-oxo-3,4-dihydro-2H-benzo[b][1,4]oxazine-6-carbonitrile

To a stirred solution of (R)-4-benzyl-2-methyl-7-nitro-3-oxo-3,4-dihydro-2H-benzo[b][1,4]oxazine-6-carbonitrile (60 mg, 0.186 mmol, 1 equiv) and NH₄C₁ (99.27 mg, 1.860 mmol, 10 equiv) in MeOH/H₂O (10:1, 5 mL) was added Fe (103.64 mg, 1.860 mmol, 10 equiv) in portions at room temperature under air atmosphere. The resulting mixture was stirred for 2 h at 70° C. under air atmosphere. Desired product could be detected by LCMS. After cooling down to rt, the reaction was quenched with water (10 mL). The resulting mixture was extracted with ethyl acetate (3×10 mL). The organic layers were combined, dried over anhydrous sodium sulfate, filtered and concentrated. The residue was purified by silica gel column chromatography, eluted with PE/EA (1:1) to afford (R)-7-amino-4-benzyl-2-methyl-3-oxo-3,4-dihydro-2H-benzo[b][1,4]oxazine-6-carbonitrile (40 mg, 58.78%) as a yellow oil. LC/MS: mass calcd for C₁₇H₁₅N₃O₂: 293.12, found: 294.05 [M+H]⁺.

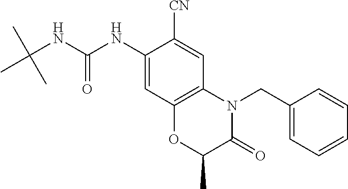 [SMILES: C[C@H]1Oc2cc(NC(=O)NC(C)(C)C)c(C#N)cc2N(Cc2ccccc2)C1=O]

### (R)-1-(4-benzyl-6-cyano-2-methyl-3-oxo-3,4-dihydro-2H-benzo[b][1,4]oxazin-7-yl)-3-(tert-butyl)urea

To a stirred solution of (R)-7-amino-4-benzyl-2-methyl-3-oxo-3,4-dihydro-2H-benzo[b][1,4]oxazine-6-carbonitrile (35 mg, 0.119 mmol, 1 equiv) and 2-isocyanato-2-methylpropane (35.49 mg, 0.357 mmol, 3 equiv) in DCM (3 mL) was added TEA (36.22 mg, 0.357 mmol, 3 equiv) in portions at 0° C. under air atmosphere. The resulting mixture was stirred for 4 h at room temperature under air atmosphere. Desired product could be detected by LCMS. The resulting mixture was extracted with CH₂Cl₂ (3×10 mL). The combined organic layers were dried over anhydrous Na₂SO₄. After filtration, the filtrate was concentrated under reduced pressure. The crude product (30 mg) was purified by Prep-HPLC with the following conditions (Column: XBridge Prep Cis OBD Column, 19*250 mm, 5 μm; Mobile Phase A: Water (10 mmol/L NH₄HCO₃), Mobile Phase B: ACN; Flow rate: 25 mL/min mL/min; Gradient: 50% B to 80% B in7 min; Wave Length: 254 nm nm; RT1(min): 5.9; Number Of Runs: 2) to afford (R)-1-(4-benzyl-6-cyano-2-methyl-3-oxo-3,4-dihydro-2H-benzo[b][1,4]oxazin-7-yl)-3-(tert-butyl)urea (23.2 mg, 49.12%) as a white solid.

LC/MS: mass calcd for C₂₂H₂₄N₄O₃: 392.18, found: 391.00 [M−H]⁻. ¹H NMR (400 MHz, DMSO-d₆) δ 8.28 (s, 1H), 7.83 (s, 1H), 7.20-7.49 (m, 6H), 6.97 (s, 1H), 5.20 (s, 2H), 4.10-5.08 (m, 1H), 1.45-1.61 (m, 3H), 1.28-1.38 (m, 9H).

### Example 342: (R)-1-(tert-butyl)-3-(6-cyano-4-(3-(difluoromethyl)benzyl)-2-methyl-3-oxo-3,4-dihydro-2H-benzo[b][1,4]oxazin-7-yl)urea

### Synthetic Scheme

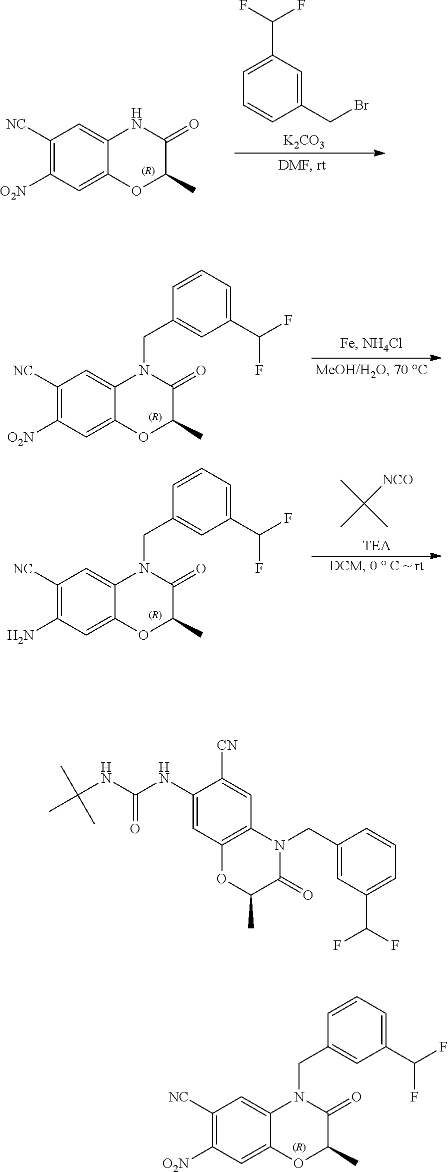

### (R)-4-(3-(difluoromethyl) benzyl)-2-methyl-7-nitro-3-oxo-3,4-dihydro-2H-benzo[b][1,4]oxazine-6-carbonitrile

To a stirred solution of (2R)-2-methyl-7-nitro-3-oxo-2,4-dihydro-1,4-benzoxazine-6-carbonitrile (91.35 mg, 0.392 mmol, 1 equiv) and 1-(bromomethyl)-3-(difluoromethyl) benzene (129.89 mg, 0.588 mmol, 1.5 equiv) in DMF (4 mL) was added K₂C₀₃ (163.61 mg, 1.176 mmol, 3 equiv) in portions at room temperature under air atmosphere. The resulting mixture was stirred for 1 h at room temperature under air atmosphere. Desired product could be detected by LCMS. The reaction was quenched with water (10 mL). The resulting mixture was extracted with ethyl acetate (3×10 mL). The organic layers were combined, dried over anhydrous sodium sulfate, filtered and concentrated. The residue was purified by silica gel column chromatography, eluted with PE/EA (1:1) to afford (R)-4-(3-(difluoromethyl) benzyl)-2-methyl-7-nitro-3-oxo-3,4-dihydro-2H-benzo[b][1,4]oxazine-6-carbonitrile (51 mg, 34.87%) as a yellow oil. LC/MS: mass calcd for C₁₈H₁₃F₂N₃O₄: 373.09, found 374.05 [M+H]⁺.

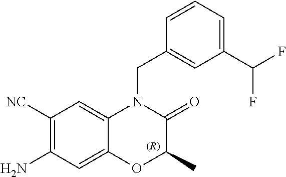

### (R)-7-amino-4-(3-(difluoromethyl) benzyl)-2-methyl-3-oxo-3,4-dihydro-2H-benzo[b][1,4]oxazine-6-carbonitrile

To a stirred mixture of (R)-4-(3-(difluoromethyl) benzyl)-2-methyl-7-nitro-3-oxo-3,4-dihydro-2H-benzo[b][1,4]oxazine-6-carbonitrile (50 mg, 0.134 mmol, 1 equiv) and Fe (74.80 mg, 1.340 mmol, 10 equiv) in MeOH/H₂O (10:1, 3 mL) was added NH₄C₁ (71.64 mg, 1.340 mmol, 10 equiv) in portions at room temperature under air atmosphere. The resulting mixture was stirred for 2 h at 70° C. under air atmosphere. Desired product could be detected by LCMS. After cooling down to rt, the reaction was quenched with water (10 mL). The resulting mixture was extracted with ethyl acetate (3×10 mL). The organic layers were combined, dried over anhydrous sodium sulfate, filtered and concentrated. The residue was purified by silica gel column chromatography, eluted with PE/EA (1:1) to afford (R)-7-amino-4-(3-(difluoromethyl) benzyl)-2-methyl-3-oxo-3,4-dihydro-2H-benzo[b][1,4]oxazine-6-carbonitrile (32 mg, 69.59%) as a yellow oil. LC/MS: mass calcd. for C₁₈H₁₅F₂N₃O₂: 343.11, found: 344.15 [M+H]⁺.

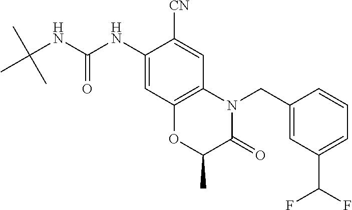 [SMILES: C[C@H]1Oc2cc(NC(=O)NC(C)(C)C)c(C#N)cc2N(Cc2cccc(C(F)F)c2)C1=O]

### (R)-1-(tert-butyl)-3-(6-cyano-4-(3-(difluoromethyl)benzyl)-2-methyl-3-oxo-3,4-dihydro-2H-benzo[b][1,4]oxazin-7-yl)urea

To a stirred solution of (R)-7-amino-4-(3-(difluoromethyl) benzyl)-2-methyl-3-oxo-3,4-dihydro-2H-benzo[b][1,4]oxazine-6-carbonitrile (25 mg, 0.081 mmol, 1 equiv) and 2-isocyanato-2-methylpropane (24.19 mg, 0.243 mmol, 3 equiv) in DCM (2 mL) was added TEA (24.69 mg, 0.243 mmol, 3 equiv) and in portions at 0° C. under air atmosphere. The resulting mixture was stirred for 4 h at room temperature under air atmosphere. Desired product could be detected by LCMS. The resulting mixture was extracted with CH₂Cl₂ (3×10 mL). The combined organic layers were dried over anhydrous Na₂SO₄. After filtration, the filtrate was concentrated under reduced pressure. The crude product (20 mg) was purified by Prep-HPLC with the following conditions (Column: XBridge Prep Cis OBD Column, 19*250 mm, 5 μm; Mobile Phase A: Water (10 mmol/L NH₄HCO₃), Mobile Phase B: ACN; Flow rate: 25 mL/min mL/min; Gradient: 50% B to 80% B in 7 min; Wave Length: 254 nm; RT1(min): 5.9; Number of Runs: 2) to afford (R)-1-(tert-butyl)-3-(6-cyano-4-(3-(difluoromethyl) benzyl)-2-methyl-3-oxo-3,4-dihydro-2H-benzo[b][1,4]oxazin-7-yl) urea (10 mg, 30.04%) as a white solid. LC/MS: mass calcd for C₂₃H₂₄F₂N₄O₃: 442.18, found: 441.05 [M−H]⁻. ¹H NMR (400 MHz, DMSO-d₆) δ 8.29 (s, 1H), 7.84 (s, 1H), 7.45-7.61 (m, 3H), 7.38-7.46 (m, 2H), 6.82-7.20 (d, J=10.7 Hz, 2H), 5.21 (s, 2H), 4.93-5.08 (q, J=6.7 Hz, 1H), 1.48-1.60 (d, J=6.8 Hz, 3H), 1.49 (s, 9H). ¹⁹F NMR (376 MHz, DMSO) δ −109.72.

### Example 343: (S)-1-(tert-butyl)-3-(2-oxo-1-(1-phenylethyl)-7-(trifluoromethyl)-1,2,3,4-tetrahydroquinolin-6-yl) urea

### Synthetic Scheme

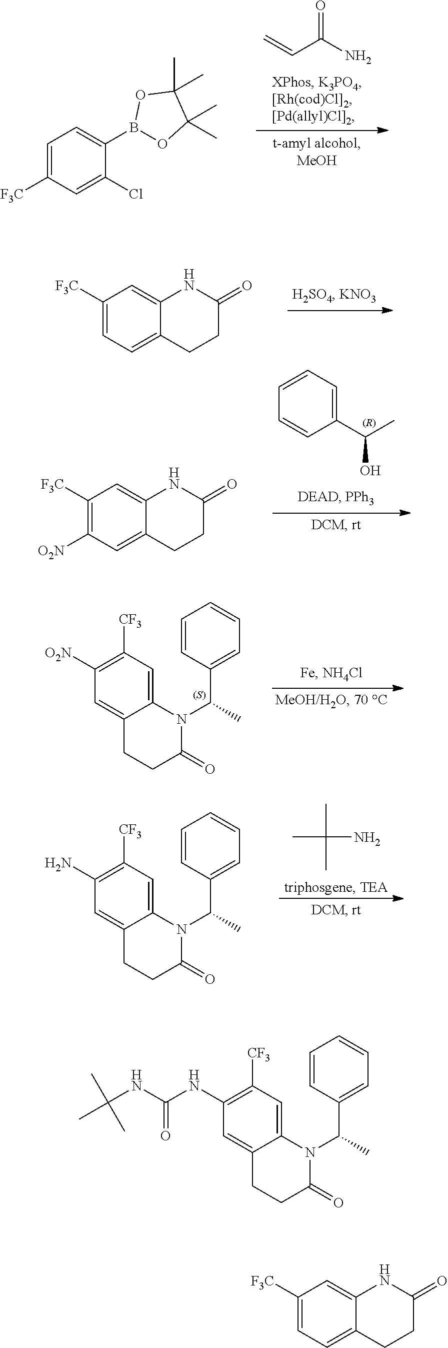

### 7-(trifluoromethyl)-3,4-dihydro-1H-quinolin-2-one

To a stirred solution of 2-[2-chloro-4-(trifluoromethyl) phenyl]-4,4,5,5-tetramethyl-1,3,2-dioxaborolane (1 g, 3.262 mmol, 1 equiv) and polyacrylamide (0.23 g, 3.262 mmol, 1 equiv), XPhos (0.62 g, 1.305 mmol, 0.4 equiv) in t-AmOH (10 mL)/MeOH (1 mL) were added K₃PO₄ (1.52 g, 7.176 mmol, 2.2 equiv), [Rh(COD)Cl]₂ (0.16 g, 0.326 mmol, 0.1 equiv) and [PdCl(allyl)]₂ (0.12 g, 0.326 mmol, 0.1 equiv) in portions at room temperature under air atmosphere. The resulting mixture was stirred for 2 h at 110° C. under nitrogen atmosphere. The mixture was allowed to cool down to room temperature. The reaction was quenched with water at room temperature. The resulting mixture was extracted with CH₂Cl₂ (3×30 mL). The combined organic layers were dried over anhydrous Na₂SO₄. After filtration, the filtrate was concentrated under reduced pressure. The residue was purified by silica gel column chromatography, eluted with PE/EA (1:1) to afford 7-(trifluoromethyl)-3,4-dihydro-1H-quinolin-2-one (610 mg, 86.90%) as a yellow oil. MS (ESI): mass calcd. For C₁₀H₈F₃NO: 215.06 m/z, found 216.20 [M+H]⁺.

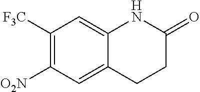 [SMILES: O=C1CCc2cc([N+](=O)[O-])c(C(F)(F)F)cc2N1]

### 6-nitro-7-(trifluoromethyl)-3,4-dihydro-1H-quinolin-2-one

A solution of 7-(trifluoromethyl)-3,4-dihydro-1H-quinolin-2-one (580 mg, 2.695 mmol, 1 equiv) and KNO₃ (272.52 mg, 2.695 mmol, 1 equiv) in H₂SO₄ (5 mL) was stirred for 2 h at room temperature under air atmosphere. The reaction was quenched with water/ice at 0° C. The precipitated solids were collected by filtration and washed with H₂O (3×20 mL). The crude product was used in the next step directly without further purification. This resulted in 6-nitro-7-(trifluoromethyl)-3,4-dihydro-1H-quinolin-2-one (570 mg, 81.28%) as a yellow solid.

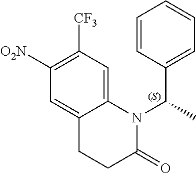

### 6-nitro-1-[(1S)-1-phenylethyl]-7-(trifluoromethyl)-3,4-dihydroquinolin-2-one

To a stirred solution of 6-nitro-7-(trifluoromethyl)-3,4-dihydro-1H-quinolin-2-one (200 mg, 0.769 mmol, 1 equiv) and 1-phenyl-ethanol (93.91 mg, 0.769 mmol, 1 equiv) in DCM (5 mL) was added PPh₃ (302.44 mg, 1.153 mmol, 1.5 equiv) in portions at room temperature under nitrogen atmosphere. The resulting mixture was stirred for 30 min at room temperature under nitrogen atmosphere. To the above mixture was added DEAD (200.82 mg, 1.153 mmol, 1.5 equiv) dropwise over 1 min at 0° C. The resulting mixture was stirred for additional 1 h at room temperature. The reaction was quenched with water at room temperature. The resulting mixture was extracted with CH₂Cl₂ (3×40 mL). The combined organic layers were dried over anhydrous Na₂SO₄. After filtration, the filtrate was concentrated under reduced pressure. The residue was purified by silica gel column chromatography, eluted with PE/EA (1:1) to afford 6-nitro-1-[(1S)-1-phenylethyl]-7-(trifluoromethyl)-3,4-dihydroquinolin-2-one (190 mg, 67.84%) as a yellow oil. MS (ESI): mass calcd. for C₁₈H₁₅F₃N₂O₃: 364.10 m/z, found 365.05 [M+H]⁺.

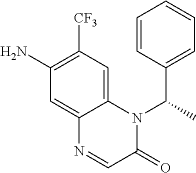 [SMILES: C[C@@H](c1ccccc1)n1c(=O)cnc2cc(N)c(C(F)(F)F)cc21]

### 6-amino-1-[(1S)-1-phenylethyl]-7-(trifluoromethyl)-3,4-dihydroquinolin-2-one

To a stirred solution of 6-nitro-1-[(1S)-1-phenylethyl]-7-(trifluoromethyl)-3,4-dihydroquinolin-2-one (190 mg, 0.522 mmol, 1 equiv) and Fe (291.24 mg, 5.220 mmol, 10 equiv) in MeOH (5 mL)/H₂O (0.5 mL) was added NH₄C₁ (278.96 mg, 5.220 mmol, 10 equiv) in portions at room temperature under air atmosphere. The resulting mixture was stirred for 1 h at 80° C. under air atmosphere. The mixture was allowed to cool down to room temperature. The reaction was quenched with water at room temperature. The resulting mixture was extracted with CH₂Cl₂ (3×30 mL). The combined organic layers were dried over anhydrous Na₂SO₄. After filtration, the filtrate was concentrated under reduced pressure. The residue was purified by silica gel column chromatography, eluted with PE/EA (1:1) to afford 6-amino-1-[(1S)-1-phenylethyl]-7-(trifluoromethyl)-3,4-dihydroquinolin-2-one (50 mg, 28.68%) as a yellow oil. MS (ESI): mass calcd. for C₁₈H₁₇F₃N₂O: 334.13 m/z, found 335.25 [M+H]⁺.

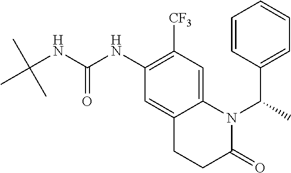 [SMILES: C[C@@H](c1ccccc1)N1C(=O)CCc2cc(NC(=O)NC(C)(C)C)c(C(F)(F)F)cc21]

### 3-tert-butyl-1-{2-oxo-1-[(1S)-1-phenylethyl]-7-(trifluoromethyl)-3,4-dihydroquinolin-6-yl}urea

A solution of 6-amino-1-[(1S)-1-phenylethyl]-7-(trifluoromethyl)-3,4-dihydroquinolin-2-one (30 mg, 0.090 mmol, 1 equiv) in DCM (5 mL) was treated with triphosgene (26.89 mg, 0.090 mmol, 1 equiv) for 1 h at 0° C. under nitrogen atmosphere followed by the addition of TEA (45.40 mg, 0.450 mmol, 5 equiv) dropwise at 0° C. The resulting mixture was stirred for 1 h at room temperature under nitrogen atmosphere. To the above mixture was added erbumine (19.69 mg, 0.270 mmol, 3 equiv) dropwise over 1 min at room temperature. The resulting mixture was stirred for additional 1 h at room temperature. The reaction was quenched with water at room temperature. The resulting mixture was extracted with CH₂Cl₂ (3×10 mL). The combined organic layers were dried over anhydrous Na₂SO₄. After filtration, the filtrate was concentrated under reduced pressure. The residue was purified by preparative HPLC using a XBridge Prep OBD C18 Column 150 mm×30 mm×5 μm column (eluent: 42% to 54% (v/v) CH₃CN and H₂O with 10 mmol/L NH₄HCO₃+0.1% NH₃·H₂O) to afford 3-tert-butyl-1-{2-oxo-1-[(1S)-1-phenylethyl]-7-(trifluoromethyl)-3,4-dihydroquinolin-6-yl}urea (18.8 mg, 48.14%) as a white solid. MS (ESI): mass calcd. for C₂₃H₂₆F₃N₃O₂: 433.20 m/z, found 434.20 [M+H]⁺. ¹H NMR (300 MHz, DMSO-d₆) δ 7.76 (s, 1H), 7.49 (s, 1H), 7.21-7.45 (m, 5H), 7.75-7.89 (m, 1H), 6.62-7.75 (m, 1H), 6.28 (q, J=7.0 Hz, 1H), 2.93 (d, J=6.7 Hz, 2H), 2.62-2.75 (m, 2H), 1.62-1.85 (m, 3H), 1.15-1.35 (m, 9H). ¹⁹F NMR (282 MHz, DMSO) δ −59.825.

### Example 344: (S)-1-(tert-butyl)-3-(7-chloro-2-oxo-1-(1-phenylethyl)-1,2-dihydroquinoxalin-6-yl)urea

### Synthetic Scheme

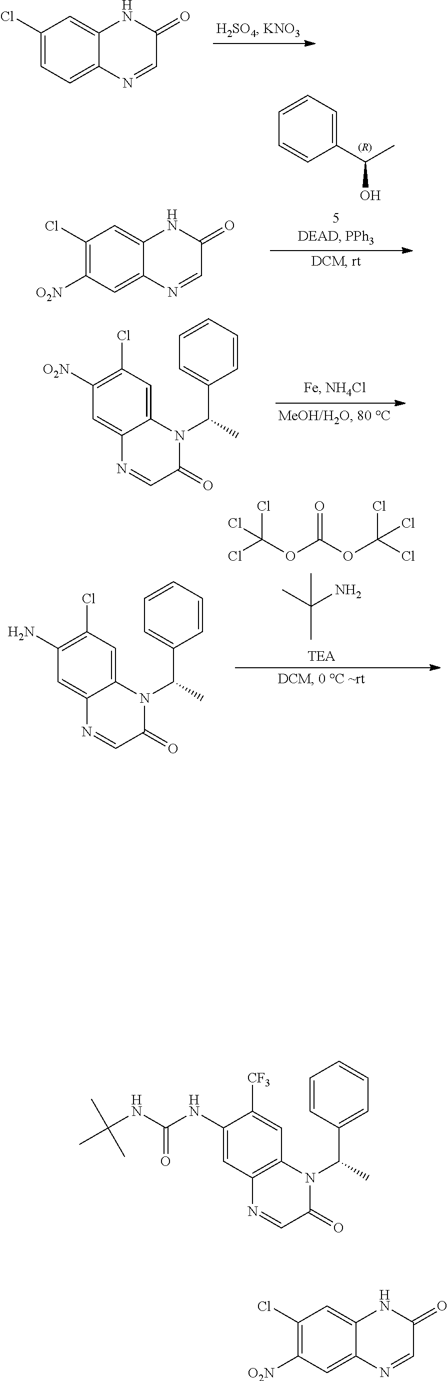

### 7-chloro-6-nitroquinoxalin-2(1H)-one

To a solution of 7-chloro-1H-quinoxalin-2-one (1 g, 5.537 mmol, 1 equiv) in H₂SO₄ (10 mL) was added KNO₃ (0.56 g, 5.537 mmol, 1 equiv) at 0° C. The reaction was stirred at 0° C. for 0.5 h. Then, the resulting mixture was stirred for 1 h at 0° C. The reaction was quenched with water/Ice at 0° C. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give 7-chloro-6-nitro-1H-quinoxalin-2-one (300 mg, 24.02%) as a yellow solid. LC/MS: mass calcd. for C₈H₄ClN₃O₃:224.99 m/z, found: 226.00 [M+H]⁺.

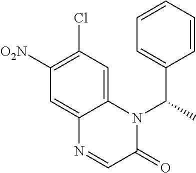 [SMILES: C[C@@H](c1ccccc1)n1c(=O)cnc2cc([N+](=O)[O-])c(Cl)cc21]

### (S)-7-chloro-6-nitro-1-(1-phenylethyl)quinoxalin-2(1H)-one

To a solution of 7-chloro-6-nitro-1H-quinoxalin-2-one (300 mg, 1.330 mmol, 1 equiv) in DCM (10 mL) was added (R)-1-phenylethan-1-ol (162.46 mg, 1.330 mmol, 1 equiv) and PPh₃ (523.21 mg, 1.995 mmol, 1.5 equiv). The reaction was stirred at rt under N₂ for 0.5 h. To the above mixture was added DEAD (347.40 mg, 1.995 mmol, 1.5 equiv) at 0° C. under N₂. The resulting mixture was stirred for additional 1 h at rt. Quenched with water (20 mL) and extracted with DCM (3×20 mL). The combined extracts were washed with water, brine, dried over Na₂SO₄, filtered and concentrated. The residue was purified by column chromatography on silica gel with EA/PE (0-100%) to give 7-chloro-6-nitro-1-[(1S)-1-phenylethyl]quinoxalin-2-one (70 mg, 15.96%) as a yellow solid. LC/MS: mass calcd. for C₁₆H₁₂ClN₃O₃:329.06 m/z, found: 330.10 [M+H]⁺.

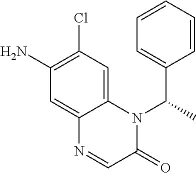 [SMILES: C[C@@H](c1ccccc1)n1c(=O)cnc2cc(N)c(Cl)cc21]

### (S)-6-amino-7-chloro-1-(1-phenylethyl)quinoxalin-2(1H)-one

To a solution of 7-chloro-6-nitro-1-[(1S)-1-phenylethyl]quinoxalin-2-one (60 mg, 0.182 mmol, 1 equiv) in MeOH (5 mL) and H₂O (0.5 mL) was added Fe (101.62 mg, 1.820 mmol, 10 equiv) and NH₄C₁ (97.33 mg, 1.820 mmol, 10 equiv). The reaction was stirred at 80° C. for 1 h. The mixture was allowed to cool down to rt. The resulting mixture was filtered, the filter cake was washed with MeOH (3×30 mL). The filtrate was concentrated under reduced pressure. Diluted with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give 6-amino-7-chloro-1-[(1S)-1-phenylethyl]quinoxalin-2-one (40 mg, 73.33%) as a yellow solid. LC/MS: mass calcd. for C₁₆H₁₄ClN₃O:299.08 m/z, found: 300.00 [M+H]⁺.

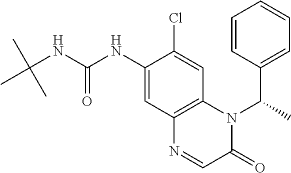 [SMILES: C[C@@H](c1ccccc1)n1c(=O)cnc2cc(NC(=O)NC(C)(C)C)c(Cl)cc21]

### (S)-1-(tert-butyl)-3-(7-chloro-2-oxo-1-(1-phenylethyl)-1,2-dihydroquinoxalin-6-yl)urea

To a solution of 6-amino-7-chloro-1-[(1S)-1-phenylethyl]quinoxalin-2-one (30 mg, 0.100 mmol, 1 equiv) in DCM (5 mL) was added triphosgene (29.70 mg, 0.100 mmol, 1 equiv) under N₂ at 0° C. The reaction was stirred at 0° C. under N₂ for 1 h. To the above mixture was added TEA (30.38 mg, 0.300 mmol, 3 equiv) at 0° C. The resulting mixture was stirred for additional 1 h at rt. Then, to the above mixture was added erbumine (31.32 mg, 0.429 mmol, 3 equiv). The resulting mixture was stirred for additional 1 h at rt. Quenched with water (10 mL) and extracted with DCM (3×10 mL). The combined extracts were washed with water, brine, dried over Na₂SO₄, filtered and concentrated. The residue was purified by preparative HPLC using a XBridge Prep OBD C18 Column, 30*150 mm, 5 m column (eluent: 38% to 68% (v/v) CH₃CN and Water (10 mmol/L NH₄HCO₃+0.05% NH₃·H₂O)) to afford the title compound 3-tert-butyl-1-{7-chloro-2-oxo-1-[(1S)-1-phenylethyl]quinoxalin-6-yl}urea (15.7 mg, 38.63%) as a yellow solid. LC/MS: mass calcd. for C₂₁H₂₃ClN₄O₂:398.15 m/z, found: 399.15 [M+H]⁺. ¹H NMR (400 MHz, DMSO-d₆) δ 8.57 (s, 1H), 8.27 (s, 1H), 7.97 (s, 1H), 7.33-7.41 (m, 2H), 7.25-7.33 (m, 3H), 7.23 (s, 1H), 6.94 (s, 1H), 6.53 (d, J=7.5 Hz, 1H), 1.87 (d, J=7.1 Hz, 3H), 1.30 (s, 9H).

### Example 345: 1-(1-benzyl-5,7-difluoro-2-oxo-1,2,3,4-tetrahydroquinolin-6-yl)-3-(tert-butyl) urea

### Synthetic Scheme

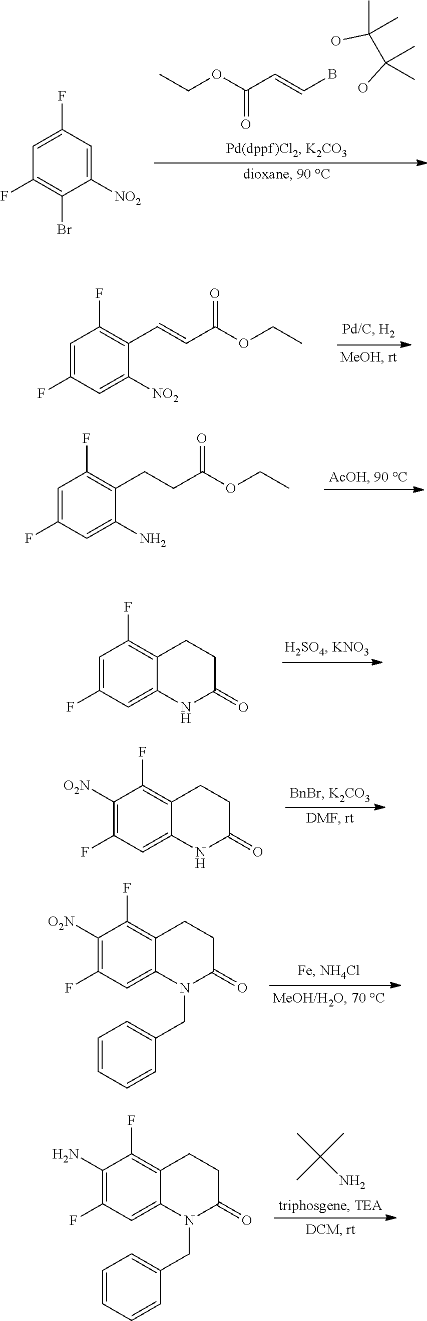

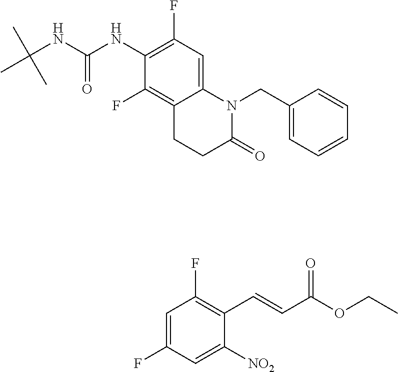 [SMILES: CC(C)(C)NC(=O)Nc1c(F)cc2c(c1F)CCC(=O)N2Cc1ccccc1 ; CCOC(=O)/C=C/c1c(F)cc(F)cc1[N+](=O)[O-]]

### ethyl (2E)-3-(2,4-difluoro-6-nitrophenyl) prop-2-enoate

To a stirred solution of 2-bromo-1,5-difluoro-3-nitrobenzene (2 g, 8.404 mmol, 1 equiv) and ethyl (2E)-3-(4,4,5,5-tetramethyl-1,3,2-dioxaborolan-2-yl) prop-2-enoate (2.28 g, 10.085 mmol, 1.2 equiv) in 1,4-dioxane (50 mL)/H₂O (5 mL) was added Pd(dppf)Cl₂ (0.68 g, 0.840 mmol, 0.1 equiv) and K₂CO₃ (2.32 g, 16.808 mmol, 2 equiv) in portions at room temperature under air atmosphere. The resulting mixture was stirred for 2 h at 90° C. under nitrogen atmosphere. The mixture was allowed to cool down to room temperature. The reaction was quenched with water at room temperature. The resulting mixture was extracted with EtOAc (3×100 mL). The combined organic layers were dried over anhydrous Na₂SO₄. After filtration, the filtrate was concentrated under reduced pressure. The residue was purified by silica gel column chromatography, eluted with PE/EA (1:1) to afford ethyl (2E)-3-(2,4-difluoro-6-nitrophenyl) prop-2-enoate (1.81 g, 83.74%) as a white oil. MS (ESI): mass calcd. For C₁₁H₉F₂NO₄: 257.05 m/z, found 258.15[M+H]⁺.

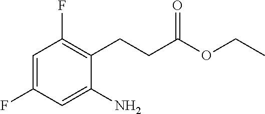 [SMILES: CCOC(=O)CCc1c(N)cc(F)cc1F]

### ethyl 3-(2-amino-4,6-difluorophenyl) propanoate

A mixture of ethyl (2E)-3-(2,4-difluoro-6-nitrophenyl) prop-2-enoate (1.81 g, 7.038 mmol, 1 equiv) and Pd/C (600 mg, 5.638 mmol, 0.80 equiv) in MeOH (10 mL) was stirred for 1 h at room temperature under hydrogen atmosphere. The resulting mixture was filtered, and the filter cake was washed with MeOH (5×20 mL). The filtrate was concentrated under reduced pressure. The crude product was used in the next step directly without further purification. This resulted in ethyl 3-(2-amino-4,6-difluorophenyl) propanoate (1.5 g, 92.98%) as a black oil. MS (ESI): mass calcd. For C₁₁H₁₃F₂NO₂: 229.09 m/z, found 230.20[M+H]⁺.

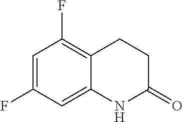 [SMILES: O=C1CCc2c(F)cc(F)cc2N1]

### 5,7-difluoro-3,4-dihydro-1H-quinolin-2-one

A solution of ethyl 3-(2-amino-4,6-difluorophenyl) propanoate (1.57 g, 6.849 mmol, 1 equiv) in AcOH (20 mL) was stirred for 1 h at 90° C. under air atmosphere. The resulting mixture was concentrated under reduced pressure. The mixture was neutralized to pH=7 with saturated NaHCO₃(aq.). The residue was purified by silica gel column chromatography, eluted with PE/EA (1:1) to afford 5,7-difluoro-3,4-dihydro-1H-quinolin-2-one (1.1 g, 87.69%) as a yellow oil. MS (ESI): mass calcd. for C₉H₇F₂NO: 183.05 m/z, found 184.25 [M+H]⁺.

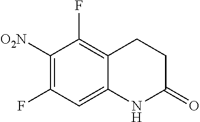 [SMILES: O=C1CCc2c(cc(F)c([N+](=O)[O-])c2F)N1]

### 5,7-difluoro-6-nitro-3,4-dihydro-1H-quinolin-2-one

A solution of 5,7-difluoro-3,4-dihydro-1H-quinolin-2-one (800 mg, 4.368 mmol, 1 equiv) and KNO₃ (441.59 mg, 4.368 mmol, 1 equiv) in H₂SO₄ (10 mL) was stirred for 1 h at room temperature under air atmosphere. The reaction was quenched with water/ice at 0° C. The precipitated solids were collected by filtration and washed with H₂O (3×20 mL). The crude product was used in the next step directly without further purification. This resulted in 5,7-difluoro-6-nitro-3,4-dihydro-1H-quinolin-2-one (790 mg, 79.27%) as a yellow oil. MS (ESI): mass calcd. for C₉H₆F₂N₂O₃: 228.03 m/z, found 229.15 [M+H]⁺.

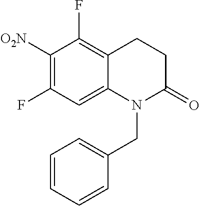 [SMILES: O=C1CCc2c(cc(F)c([N+](=O)[O-])c2F)N1Cc1ccccc1]

### 1-benzyl-5,7-difluoro-6-nitro-3,4-dihydroquinolin-2-one

To a stirred solution of 5,7-difluoro-6-nitro-3,4-dihydro-1H-quinolin-2-one (500 mg, 2.191 mmol, 1 equiv) and BnBr (374.83 mg, 2.191 mmol, 1 equiv) in DMF (5 mL) was added K₂CO₃ (605.75 mg, 4.382 mmol, 2 equiv) in portions at room temperature under air atmosphere. The resulting mixture was stirred for 1 h at room temperature under air atmosphere. The reaction was quenched with water at room temperature. The resulting mixture was extracted with EtOAc (3×30 mL). The combined organic layers were dried over anhydrous Na₂SO₄. After filtration, the filtrate was concentrated under reduced pressure. The residue was purified by silica gel column chromatography, eluted with PE/EA (1:1) to afford 1-benzyl-5,7-difluoro-6-nitro-3,4-dihydroquinolin-2-one (200 mg, 28.67%) as a yellow oil. MS (ESI): mass calcd. for C₁₆H₁₂F₂N₂O₃: 318.08 m/z, found 319.20 [M+H]⁺.

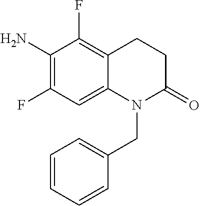 [SMILES: Nc1c(F)cc2c(c1F)CCC(=O)N2Cc1ccccc1]

### 6-amino-1-benzyl-5,7-difluoro-3,4-dihydroquinolin-2-one

To a stirred solution of 1-benzyl-5,7-difluoro-6-nitro-3,4-dihydroquinolin-2-one (150 mg, 0.471 mmol, 1 equiv) and Fe (263.19 mg, 4.710 mmol, 10 equiv) in MeOH (5 mL)/H₂O (0.5 mL) was added NH₄C₁ (252.09 mg, 4.710 mmol, 10 equiv) in portions at room temperature under air atmosphere. The resulting mixture was stirred for 1 h at 80° C. under air atmosphere. The mixture was allowed to cool down to room temperature. The reaction was quenched with water at room temperature. The resulting mixture was extracted with EtOAc (3×30 mL). The combined organic layers were dried over anhydrous Na₂SO₄. After filtration, the filtrate was concentrated under reduced pressure. The residue was purified by silica gel column chromatography, eluted with PE/EA (1:1) to afford 6-amino-1-benzyl-5,7-difluoro-3,4-dihydroquinolin-2-one (120 mg, 88.32%) as a yellow solid. MS (ESI): mass calcd. for C₁₆H₁₄F₂N₂O: 288.11 m/z, found 289.25 [M+H]⁺.

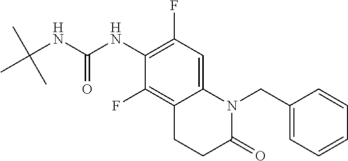 [SMILES: CC(C)(C)NC(=O)Nc1c(F)cc2c(c1F)CCC(=O)N2Cc1ccccc1]

### 1-(1-benzyl-5,7-difluoro-2-oxo-3,4-dihydroquinolin-6-yl)-3-tert-butylurea

A solution of 6-amino-1-benzyl-5,7-difluoro-3,4-dihydroquinolin-2-one (110 mg, 0.382 mmol, 1 equiv) in DCM (5 mL) was treated with triphosgene (113.22 mg, 0.382 mmol, 1 equiv) for 1 h at 0° C. under nitrogen atmosphere followed by the addition of TEA (193.05 mg, 1.910 mmol, 5 equiv) dropwise at 0° C. The resulting mixture was stirred for 1 h at room temperature under nitrogen atmosphere. To the above mixture was added erbumine (67.66 mg, 1.146 mmol, 3 equiv) dropwise over 1 min at room temperature. The resulting mixture was stirred for additional 1 h at room temperature. The reaction was quenched with water at room temperature. The resulting mixture was extracted with CH₂Cl₂ (3×30 mL). The combined organic layers were dried over anhydrous Na₂SO₄. After filtration, the filtrate was concentrated under reduced pressure. The residue was purified by preparative HPLC using a XBridge Prep OBD C18 Column 150 mm×30 mm×5 m column (eluent: 40% to 50% (v/v) CH₃CN and H₂O with 10 mmol/L NH₄HCO₃+0.1% NH₃·H₂O) to afford 1-(1-benzyl-5,7-difluoro-2-oxo-3,4-dihydroquinolin-6-yl)-3-tert-butylurea (52.7 mg, 35.36%) as a white solid. MS (ESI): mass calcd. for C₂₁H₂₃F₂N₃O₂: 387.18 m/z, found 388.20 [M+H]⁺. ¹H NMR (300 MHz, DMSO-d₆) δ 7.40 (s, 1H), 7.28-7.38 (m, 2H), 7.24 (td, J=4.9, 2.8 Hz, 3H), 6.72 (dd, J=12.0, 1.8 Hz, 1H), 6.11 (s, 1H), 5.16 (s, 2H), 2.91 (t, J=7.4 Hz, 2H), 2.73 (dd, J=8.7, 6.1 Hz, 2H), 1.20-1.30 (m, 9H). ¹⁹F NMR (282 MHz, DMSO) δ −120.687, −123.203.

### Example 346: 3-tert-butyl-1-{2-oxo-1-[(1S)-1-phenylethyl]-7-(trifluoromethyl)quinoxalin-6-yl}urea

### Synthetic Scheme

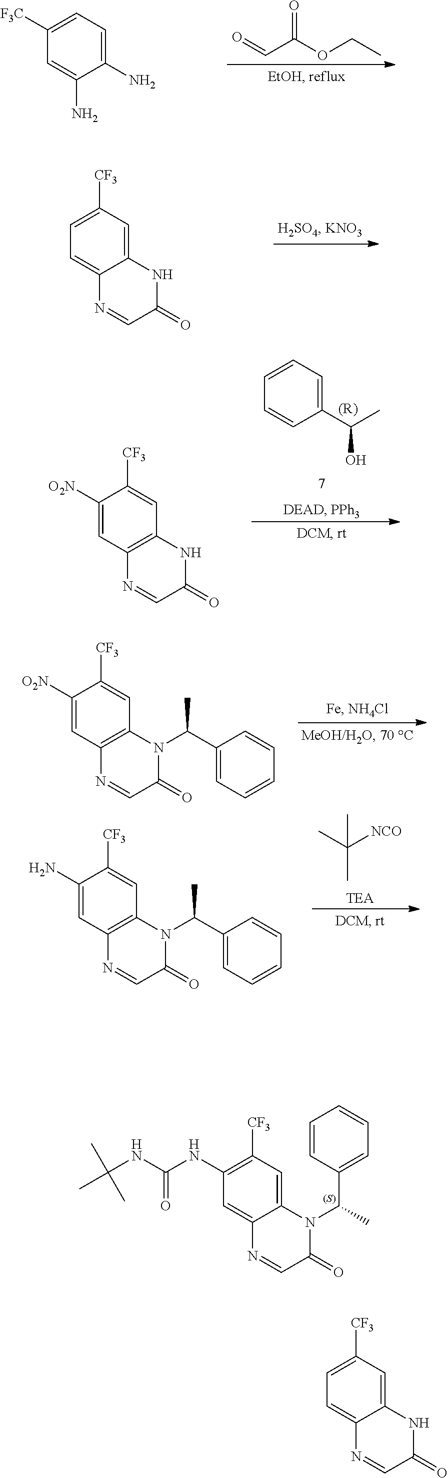

### Synthesis of 7-(trifluoromethyl)-1H-quinoxalin-2-one

To a stirred solution of 4-(trifluoromethyl)benzene-1,2-diamine (2 g, 11.354 mmol, 1 equiv) in EtOH (20 mL) was added ethyl glyoxylate (1.16 g, 11.354 mmol, 1 equiv) in portions at room temperature under air atmosphere. The resulting mixture was stirred for 3 h at 60° C. under air atmosphere. The reaction was monitored by LCMS. Quenched with water (100 mL) and extracted with EA (3×100 mL). The combined extracts were washed with water, brine, dried over Na₂SO₄, filtered and concentrated. The residue was purified by silica gel column chromatography, eluted with PE/EA (0-100%) to afford 7-(trifluoromethyl)-1H-quinoxalin-2-one (1 g, 41.13%) as a yellow solid. LC/MS (ESI): mass calcd. for C₉H₅F₃N₂O: 214.04 m/z, found: 215.0 [M+H]⁺.

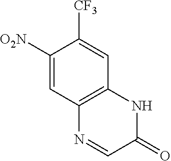 [SMILES: O=c1cnc2cc([N+](=O)[O-])c(C(F)(F)F)cc2[nH]1]

### Synthesis of 6-nitro-7-(trifluoromethyl)-1H-quinoxalin-2-one

To a stirred solution of 7-(trifluoromethyl)-1H-quinoxalin-2-one (1 g, 4.670 mmol, 1 equiv) in H₂SO₄ (10 mL) was added KNO₃ (1.42 g, 14.010 mmol, 3 equiv) in portions at 0° C. under air atmosphere. The resulting mixture was stirred for 2 h at room temperature under air atmosphere. The reaction was monitored by LCMS. The reaction was quenched with water/Ice at 0° C. The precipitated solids were collected by filtration and washed with H₂O (3×20 mL). The crude product was used in the next step directly without further purification. This resulted in 6-nitro-7-(trifluoromethyl)-1H-quinoxalin-2-one (600 mg, 49.58%) as a brown solid. LC/MS (ESI): mass calcd. for C₉H₄F₃N₃O₃:259.02 m/z, found: 260.0 [M+H]⁺.

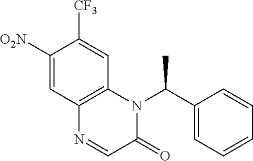 [SMILES: C[C@@H](c1ccccc1)n1c(=O)cnc2cc([N+](=O)[O-])c(C(F)(F)F)cc21]

### Synthesis of 6-nitro-1-[(1S)-1-phenylethyl]-7-(trifluoromethyl)quinoxalin-2-one

To a stirred solution of 6-nitro-7-(trifluoromethyl)-1H-quinoxalin-2-one (590 mg, 2.277 mmol, 1 equiv) and 1-phenyl-ethanol (278.14 mg, 2.277 mmol, 1 equiv) in DCM (6 mL) was added PPh₃ (895.75 mg, 3.416 mmol, 1.5 equiv) in portions at 0° C. under nitrogen atmosphere. The resulting mixture was stirred for 15 min at room temperature under nitrogen atmosphere. To the above mixture was added DEAD (594.76 mg, 3.416 mmol, 1.5 equiv) in portions at 0° C. The resulting mixture was stirred for additional 2 h at room temperature. The reaction was monitored by LCMS. Quenched with water (50 mL) and extracted with DCM (3×50 mL). The combined extracts were washed with water, brine, dried over Na₂SO₄, filtered and concentrated. The residue was purified by silica gel column chromatography, eluted with PE/EA (0-100%) to afford 6-nitro-1-[(1S)-1-phenylethyl]-7-(trifluoromethyl)quinoxalin-2-one (100 mg, 12.09%) as a yellow solid. LC/MS (ESI): mass calcd. for C₁₇H₁₂F₃N₃O₃:363.0 m/z, found: 364.0 [M+H]⁺.

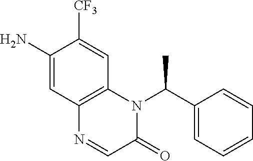 [SMILES: C[C@@H](c1ccccc1)n1c(=O)cnc2cc(N)c(C(F)(F)F)cc21]

### Synthesis of 6-amino-1-[(1S)-1-phenylethyl]-7-(trifluoromethyl)quinoxalin-2-one

To a stirred solution of 6-nitro-1-[(1S)-1-phenylethyl]-7-(trifluoromethyl)quinoxalin-2-one (90 mg, 0.248 mmol, 1 equiv) in MeOH/H₂O (10:1, 1.1 mL) was added Fe (15.37 mg, 0.280 mmol, 10 equiv) and NH₄C₁ (132.51 mg, 2.480 mmol, 10 equiv) in portions at room temperature under air atmosphere. The resulting mixture was stirred for 2 h at 70° C. under air atmosphere. The reaction was monitored by LCMS. After cooling down to rt, the reaction was quenched with water (10 mL) and extracted with EA (3×10 mL). The combined extracts were washed with water, brine, dried over Na₂SO₄, filtered and concentrated. The residue was purified by silica gel column chromatography, eluted with PE/EA (0-100%) to afford 6-amino-1-[(1S)-1-phenylethyl]-7-(trifluoromethyl)quinoxalin-2-one (30 mg, 36.33%) as a yellow solid. LC/MS (ESI): mass calcd. for C₁₇H₁₄F₃N₃O:333.1 m/z, found: 334.1 [M+H]⁺.

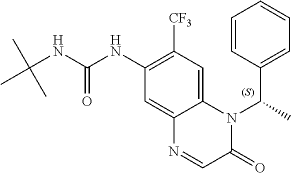

### Synthesis of 3-tert-butyl-1-{2-oxo-1-[(1S)-1-phenylethyl]-7-(trifluoromethyl)quinoxalin-6-yl}urea

To a stirred solution of 6-amino-7-methyl-1-[(1S)-1-phenylethyl]quinoxalin-2-one (40 mg, 0.143 mmol, 1 equiv) in DCM (0.5 mL) was added TEA (22.77 mg, 0.225 mmol, 3 equiv) and 2-isocyanato-2-methylpropane (22.31 mg, 0.225 mmol, 3 equiv) at room temperature under air atmosphere. The resulting mixture was stirred for 1 days at room temperature under air atmosphere. The reaction was monitored by LCMS. Quenched with water (5 mL) and extracted with DCM (3×5 mL). The combined extracts were washed with water, brine, dried over Na₂SO₄, filtered and concentrated. The crude product was purified by Prep-HPLC using a X Bridge Prep OBD C18 150 mm×30 mm×5 μm column (eluent: 42% to 72% (v/v) CH₃CN and H₂O with 10 mmol/L NH₄HCO₃+0.05% NH₃H₂O) to afford 3-tert-butyl-1-{7-methyl-2-oxo-1-[(1S)-1-phenylethyl]quinoxalin-6-yl}urea (2.7 mg, 4.96%) as a yellow solid. LC/MS (ESI): mass calcd. for C₂₂H₂₃F₃N₄O₂:432.2 m/z, found: 433.15 [M+H]⁺. ¹H NMR (300 MHz, DMSO-d₆) δ 8.41 (s, 1H), 8.34 (s, 1H), 7.65 (s, 1H), 7.37-7.31 (m, 6H), 6.90 (s, 1H), 6.62 (q, J=7.2 Hz, 1H), 1.86 (d, J=7.2 Hz, 3H), 1.28 (s, 9H). ¹⁹F NMR (282 MHz, DMSO-d₆) δ −60.29.

### Example 347: 3-tert-butyl-1-{7-methyl-2-oxo-1-[(1S)-1-phenylethyl]quinoxalin-6-yl}urea

### Synthetic Scheme

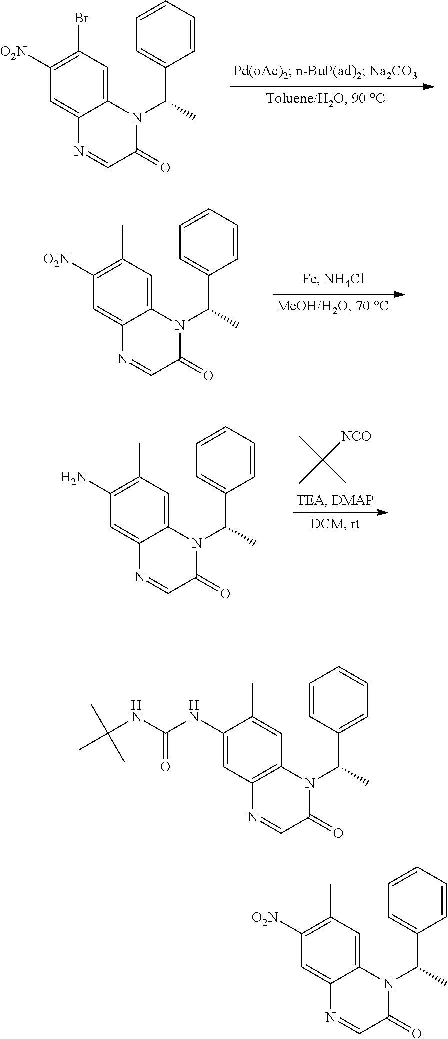

### 7-methyl-6-nitro-1-[(1S)-1-phenylethyl]quinoxalin-2-one

To a stirred solution of 7-bromo-6-nitro-1-[(1S)-1-phenylethyl]quinoxalin-2-one (200 mg, 0.534 mmol, 1 equiv) and methylboronic acid (63.99 mg, 1.068 mmol, 2 equiv) in toluene/H₂O (7:1, 2.4 mL) was added Pd(OAc)₂ (24.00 mg, 0.107 mmol, 0.2 equiv), Butyldi-1-adamantylphosphine (1.92 mg, 0.005 mmol, 0.2 equiv) and Na₂CO₃ (169.95 mg, 1.602 mmol, 3 equiv) in portions at room temperature under nitrogen atmosphere. The resulting mixture was stirred for 3 h at 90° C. under nitrogen atmosphere. The reaction was monitored by LCMS. The mixture was allowed to cool down to room temperature. Quenched with water (10 mL) and extracted with EA (3×10 mL). The combined extracts were washed with water, brine, dried over Na₂SO₄, filtered and concentrated. The residue was purified by silica gel column chromatography, eluted with PE/EA (0-100%) to afford 7-methyl-6-nitro-1-[(1S)-1-phenylethyl]quinoxalin-2-one (100 mg, 60.49%) as a yellow solid. LC/MS (ESI): mass calcd. for C₁₇H₁₅N₃O₃:309.1 m/z, found: 310.0 [M+H]⁺.

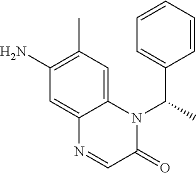 [SMILES: Cc1cc2c(cc1N)ncc(=O)n2[C@@H](C)c1ccccc1]

### 6-amino-7-methyl-1-[(1S)-1-phenylethyl]quinoxalin-2-one

To a stirred solution of 7-methyl-6-nitro-1-[(1S)-1-phenylethyl]quinoxalin-2-one (90 mg, 0.291 mmol, 1 equiv) in MeOH/H₂O (10:1, 1.1 mL) was added Fe (18.05 mg, 0.320 mmol, 10 equiv) and NH₄C₁ (155.63 mg, 2.910 mmol, 10 equiv) in portions at room temperature under air atmosphere. The resulting mixture was stirred for 2 h at 70° C. under air atmosphere. The reaction was monitored by LCMS. After cooling down to rt, quenched with water (10 mL) and extracted with EA (3×10 mL). The combined extracts were washed with water, brine, dried over Na₂SO₄, filtered and concentrated. The residue was purified by silica gel column chromatography, eluted with PE/EA (0-100%) to afford 6-amino-7-methyl-1-[(1S)-1-phenylethyl]quinoxalin-2-one (70 mg, 86.13%) as a yellow solid. LC/MS (ESI): mass calcd. for C₁₇H₁₇N₃O: 279.14 m/z, found: 280.1 [M+H]⁺.

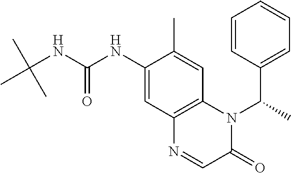 [SMILES: Cc1cc2c(cc1NC(=O)NC(C)(C)C)ncc(=O)n2[C@@H](C)c1ccccc1]

### 3-tert-butyl-1-{7-methyl-2-oxo-1-[(1S)-1-phenylethyl]quinoxalin-6-yl}urea

To a stirred solution of 6-amino-7-methyl-1-[(1S)-1-phenylethyl]quinoxalin-2-one (80 mg, 0.286 mmol, 1 equiv) in DCM (1 mL) was added 2-isocyanato-2-methylpropane (85.17 mg, 0.858 mmol, 3 equiv) and TEA (86.94 mg, 0.858 mmol, 3 equiv) in portions at room temperature under air atmosphere. The resulting mixture was stirred for 1 days at room temperature under air atmosphere. The reaction was monitored by LCMS. Quenched with water (10 mL) and extracted with EA (3×10 mL). The combined extracts were washed with water, brine, dried over Na₂SO₄, filtered and concentrated. The crude product was purified by Prep-HPLC using XSelect CSH Fluoro Pheny 250 mm×19 mm×5 m column (eluent: 38% to 68% (v/v) CH₃CN and H₂O with 0.1% FA) to afford 3-tert-butyl-1-{7-methyl-2-oxo-1-[(1S)-1-phenylethyl]quinoxalin-6-yl}urea (4.1 mg, 3.76%) as a yellow solid. LC/MS (ESI): mass calcd. for C₂₂H₂₆N₄O₂:378.2 m/z, found: 379.15 [M+H]⁺. ¹H NMR (300 MHz, DMSO-d₆) δ 8.34 (s, 1H), 8.18 (s, 1H), 7.56 (s, 1H), 7.37-7.24 (m, 5H), 7.07 (s, 1H), 6.55 (d, J=8.4 Hz, 2H), 2.12 (s, 3H), 1.89 (d, J=6.9 Hz, 3H), 1.29 (s, 9H).

### Example 348: 3-tert-butyl-1-{7-cyclopropyl-2-oxo-1-[(1S)-1-phenylethyl]quinoxalin-6-yl}urea

### Synthetic Scheme

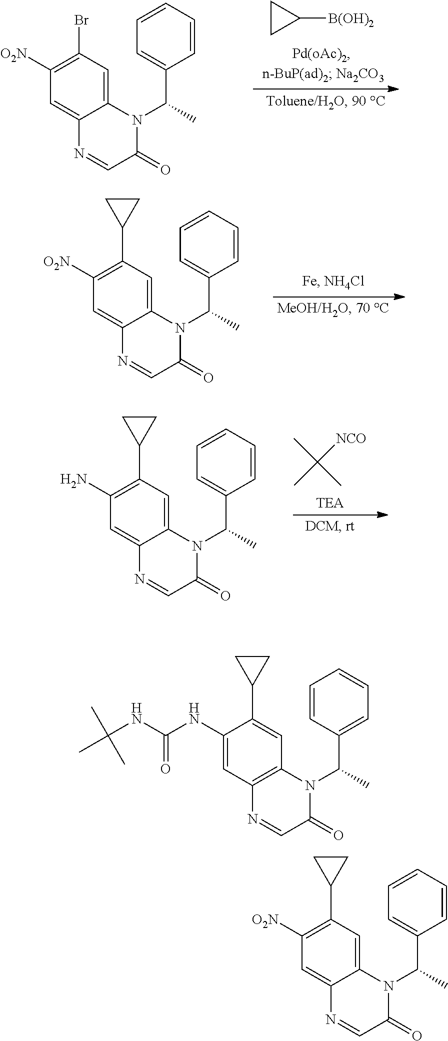

### 7-cyclopropyl-6-nitro-1-[(1S)-1-phenylethyl]quinoxalin-2-one

To a stirred solution of 7-bromo-6-nitro-1-[(1S)-1-phenylethyl]quinoxalin-2-one (200 mg, 0.534 mmol, 1 equiv) and cyclopropylboronic acid (229.56 mg, 2.670 mmol, 5 equiv) in toluene/H₂O (7:1, 2.4 mL) were added Na₂CO₃ (169.95 mg, 1.602 mmol, 3 equiv), Pd(OAc)₂ (24.00 mg, 0.107 mmol, 0.2 equiv) and Butyldi-1-adamantylphosphine (38.33 mg, 0.107 mmol, 0.2 equiv) in portions at room temperature under nitrogen atmosphere. The resulting mixture was stirred for 3 h at 90° C. under nitrogen atmosphere. The reaction was monitored by LCMS. The mixture was allowed to cool down to room temperature. Quenched with water (10 mL) and extracted with EA (3×10 mL). The combined extracts were washed with water, brine, dried over Na₂SO₄, filtered and concentrated. The residue was purified by silica gel column chromatography, eluted with PE/EA (0-100%) to afford 7-cyclopropyl-6-nitro-1-[(1S)-1-phenylethyl]quinoxalin-2-one as a yellow solid. LC/MS (ESI): mass calcd. for C₁₉H₁₇N₃O₃:335.13 m/z, found: 336.0 [M+H]⁺.

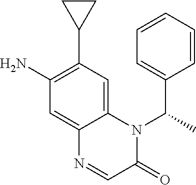 [SMILES: C[C@@H](c1ccccc1)n1c(=O)cnc2cc(N)c(C3CC3)cc21]

### 6-amino-7-cyclopropyl-1-[(1S)-1-phenylethyl]quinoxalin-2-one

To a stirred solution of 7-cyclopropyl-6-nitro-1-[(1S)-1-phenylethyl]quinoxalin-2-one (90 mg, 0.268 mmol, 1 equiv) in MeOH/H₂O (10:1, 1.1 mL) was added Fe (16.65 mg, 0.300 mmol, 10 equiv) and NH₄C₁ (143.55 mg, 2.680 mmol, 10 equiv) in portions at room temperature under air atmosphere. The resulting mixture was stirred for 2 h at 70° C. under air atmosphere. The reaction was monitored by LCMS. The mixture was allowed to cool down to room temperature. Quenched with water (8 mL) and extracted with EA (3×8 mL). The combined extracts were washed with water, brine, dried over Na₂SO₄, filtered and concentrated. The residue was purified by silica gel column chromatography, eluted with PE/EA (0-100%) to afford 6-amino-7-cyclopropyl-1-[(1S)-1-phenylethyl]quinoxalin-2-one (80 mg, 97.62%) as a yellow solid. LC/MS (ESI): mass calcd. for C₁₉H₁₉N₃O: 305.15 m/z, found: 306.05 [M+H]⁺.

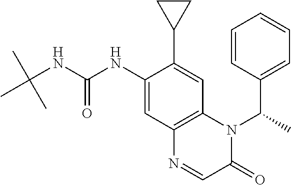 [SMILES: C[C@@H](c1ccccc1)n1c(=O)cnc2cc(NC(=O)NC(C)(C)C)c(C3CC3)cc21]

### Synthesis of 3-tert-butyl-1-{7-cyclopropyl-2-oxo-1-[(1S)-1-phenylethyl]quinoxalin-6-yl}urea

To a stirred solution of 6-amino-7-cyclopropyl-1-[(1S)-1-phenylethyl]quinoxalin-2-one (80 mg, 0.262 mmol, 1 equiv) and TEA (79.53 mg, 0.786 mmol, 3 equiv) in DCM (1 mL) was added 2-isocyanato-2-methylpropane (77.91 mg, 0.786 mmol, 3 equiv) in portions at room temperature under air atmosphere. The resulting mixture was stirred for 1 days at room temperature under air atmosphere. The reaction was monitored by LCMS. Quenched with water (8 mL) and extracted with DCM (3×8 mL). The combined extracts were washed with water, brine, dried over Na₂SO₄, filtered and concentrated. The crude product was purified by Prep-HPLC using a X Bridge Prep OBD C18 150 mm×30 mm×5 μm column (eluent: 36% to 46% (v/v) CH₃CN and H₂O with 10 mmol/L NH₄HCO_{3+0.05}% NH₃H₂O) to afford 3-tert-butyl-1-{7-cyclopropyl-2-oxo-1-[(1S)-1-phenylethyl]quinoxalin-6-yl}urea (16.7 mg, 15.65%) as a yellow solid. LC/MS (ESI): mass calcd. for C₂₄H₂₈N₄O₂:404.2 m/z, found: 405.2 [M+H]⁺. ¹H NMR (300 MHz, DMSO-d₆) δ 8.32 (s, 1H), 8.21 (s, 1H), 7.71 (s, 1H), 7.40-7.36 (m, 2H), 7.35-7.26 (m, 3H), 6.64 (s, 2H), 6.52 (s, 1H), 1.84 (d, J=7.2 Hz, 3H), 1.80-1.75 (m, 1H), 1.30 (s, 9H), 0.90-0.87 (m, 2H), 0.28-0.01 (m, 2H).

### Example 349: 1-(tert-butyl)-3-((R)-2-methyl-3-oxo-4-((S)-1-phenylethyl)-6-(trifluoromethyl)-3,4-dihydro-2H-benzo[b][1,4]oxazin-7-yl)urea

### Synthetic Scheme

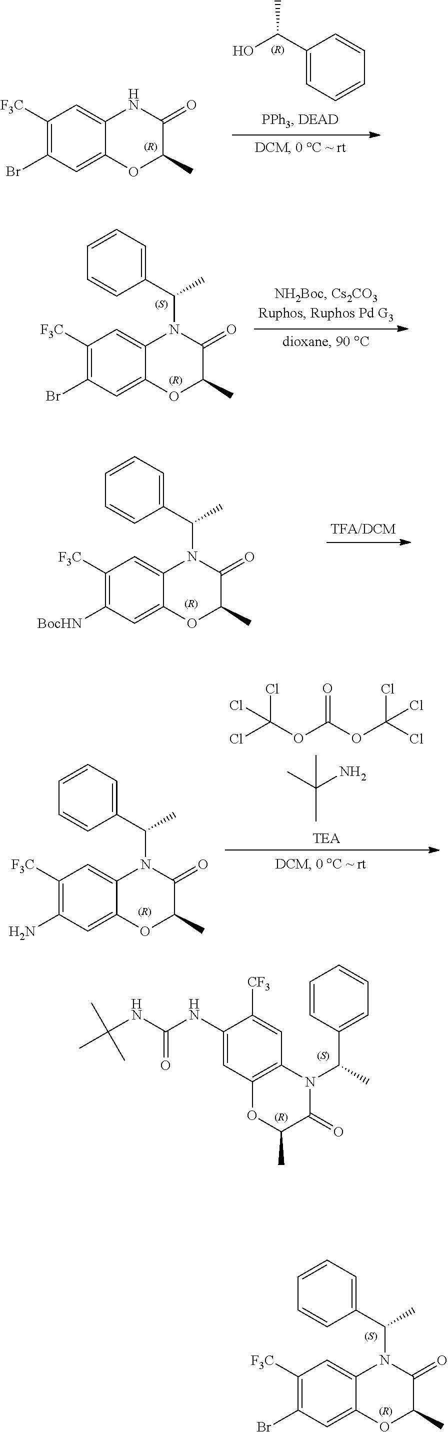

### (R)-7-bromo-2-methyl-4-((S)-1-phenylethyl)-6-(trifluoromethyl)-2H-benzo[b][1,4]oxazin-3(4H)-one

To a solution of (2R)-7-bromo-2-methyl-6-(trifluoromethyl)-2,4-dihydro-1,4-benzoxazin-3-one (350 mg, 1.129 mmol, 1 equiv) in DCM (10 mL) was added (R)-1-phenylethan-1-ol (137.90 mg, 1.129 mmol, 1 equiv) and PPh₃ (444.10 mg, 1.694 mmol, 1.5 equiv). The reaction was stirred at rt under N₂ for 0.5 h. To the above mixture was added DEAD (294.88 mg, 1.694 mmol, 1.5 equiv) at 0° C. under N₂. The resulting mixture was stirred for additional 1 h at rt. Quenched with water (30 mL) and extracted with DCM (3×30 mL). The combined extracts were washed with water, brine, dried over Na₂SO₄, filtered and concentrated. The residue was purified by column chromatography on silica gel with EA/PE (0-100%) to give (2R)-7-bromo-2-methyl-4-[(1S)-1-phenylethyl]-6-(trifluoromethyl)-2H-1,4-benzoxazin-3-one (240 mg, 51.33%) as a yellow solid. LC/MS: mass calcd. for C₁₈H₁₅BrF₃NO₂:413.02 m/z, found: 413.95, 415.95 [M+H, M+H+2]⁺.

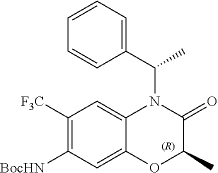

### tert-butyl ((R)-2-methyl-3-oxo-4-((S)-1-phenylethyl)-6-(trifluoromethyl)-3,4-dihydro-2H-benzo[b][1,4]oxazin-7-yl)carbamate

To a solution of (2R)-7-bromo-2-methyl-4-[(1S)-1-phenylethyl]-6-(trifluoromethyl)-2H-1,4-benzoxazin-3-one (260 mg, 0.628 mmol, 1 equiv) in dioxane (10 mL) was added tert-butyl carbamate (110.30 mg, 0.942 mmol, 1.5 equiv), Ruphos (29.29 mg, 0.063 mmol, 0.1 equiv), RuPhos Palladacycle Gen.3 (52.50 mg, 0.063 mmol, 0.1 equiv) and Cs₂CO₃ (613.53 mg, 1.884 mmol, 3 equiv). The reaction was stirred at 90° C. under N₂ for 1 h. The mixture was allowed to cool down to rt. Quenched with water (30 mL) and extracted with EA (3×30 mL). The combined extracts were washed with water, brine, dried over Na₂SO₄, filtered and concentrated. The residue was purified by column chromatography on silica gel with EA/PE (0-100%) to give tert-butyl N-[(2R)-2-methyl-3-oxo-4-[(1S)-1-phenylethyl]-6-(trifluoromethyl)-2H-1,4-benzoxazin-7-yl]carbamate (250 mg, 88.42%) as a yellow solid. LC/MS: mass calcd. for C₂₃H₂₅F₃N₂O₄:450.18 m/z, found: 451.15 [M+H]⁺.

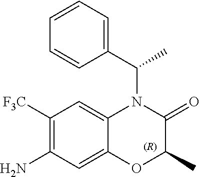

### (R)-7-amino-2-methyl-4-((S)-1-phenylethyl)-6-(trifluoromethyl)-2H-benzo[b][1,4]oxazin-3(4H)-one

To a solution of tert-butyl N-[(2R)-2-methyl-3-oxo-4-[(1S)-1-phenylethyl]-6-(trifluoromethyl)-2H-1,4-benzoxazin-7-yl]carbamate (270 mg, 0.599 mmol, 1 equiv) in DCM (4 mL) was added TFA (1 mL) at 0° C. The reaction was stirred at rt for 1 h. Quenched with NaHCO₃ solution (20 mL) and extracted with DCM (3×20 mL). The combined extracts were washed with water, brine, dried over Na₂SO₄, filtered and concentrated. The residue was purified by column chromatography on silica gel with EA/PE (0-100%) to give (2R)-7-amino-2-methyl-4-[(1S)-1-phenylethyl]-6-(trifluoromethyl)-2H-1,4-benzoxazin-3-one (100 mg, 47.62%) as a yellow solid. LC/MS: mass calcd. for C₁₈H₁₇F₃N₂O₂:350.12 m/z, found: 351.05 [M+H]f.

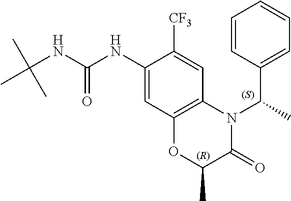

### 1-(tert-butyl)-3-((R)-2-methyl-3-oxo-4-((S)-1-phenylethyl)-6-(trifluoromethyl)-3,4-dihydro-2H-benzo[b][1,4]oxazin-7-yl)urea

To a solution of (2R)-7-amino-2-methyl-4-[(1S)-1-phenylethyl]-6-(trifluoromethyl)-2H-1,4-benzoxazin-3-one (50 mg, 0.143 mmol, 1 equiv) in DCM (5 mL) was added triphosgene (42.35 mg, 0.143 mmol, 1 equiv) under N₂ at 0° C. The reaction was stirred at 0° C. under N₂ for 1 h. To the above mixture was added TEA (43.33 mg, 0.429 mmol, 3 equiv) at 0° C. The resulting mixture was stirred for additional 1 h at rt. Then, to the above mixture was added erbumine (31.31 mg, 0.429 mmol, 3 equiv). The resulting mixture was stirred for additional 1 h at rt. Quenched with water (10 mL) and extracted with DCM (3×10 mL). The combined extracts were washed with water, brine, dried over Na₂SO₄, filtered and concentrated. The residue was purified by preparative HPLC using a XSelect CSH C18 Column, 19*250 mm, 5 μm column (eluent: 58% to 88% (v/v) CH₃CN and Water (0.1% FA)) to afford the title compound 3-tert-butyl-1-[(2R)-2-methyl-3-oxo-4-[(1S)-1-phenylethyl]-6-(trifluoromethyl)-2H-1,4-benzoxazin-7-yl]urea (26.1 mg, 40.55%) as a white solid. LC/MS: mass calcd. for C₂₃H₂₆F₃N₃O₃:449.19 m/z, found: 450.15 [M+H]⁺. ¹H NMR (400 MHz, DMSO-d₆) δ 7.69 (s, 1H), 7.54 (s, 1H), 7.35-7.43 (m, 2H), 7.29 (d, J=7.3 Hz, 3H), 6.93 (s, 1H), 6.82 (s, 1H), 6.19 (q, J=7.1 Hz, 1H), 4.92 (q, J=6.8 Hz, 1H), 1.75 (d, J=7.2 Hz, 3H), 1.50 (d, J=6.8 Hz, 3H), 1.27 (s, 9H). ¹⁹F NMR (376 MHz, DMSO) δ(ppm): −58.9959.

### Example 350: (R)-1-(tert-butyl)-3-(4-(3-(difluoromethyl)benzyl)-8-fluoro-2-methyl-3-oxo-6-(trifluoromethyl)-3,4-dihydro-2H-benzo[b][1,4]oxazin-7-yl)urea

### Synthetic Scheme

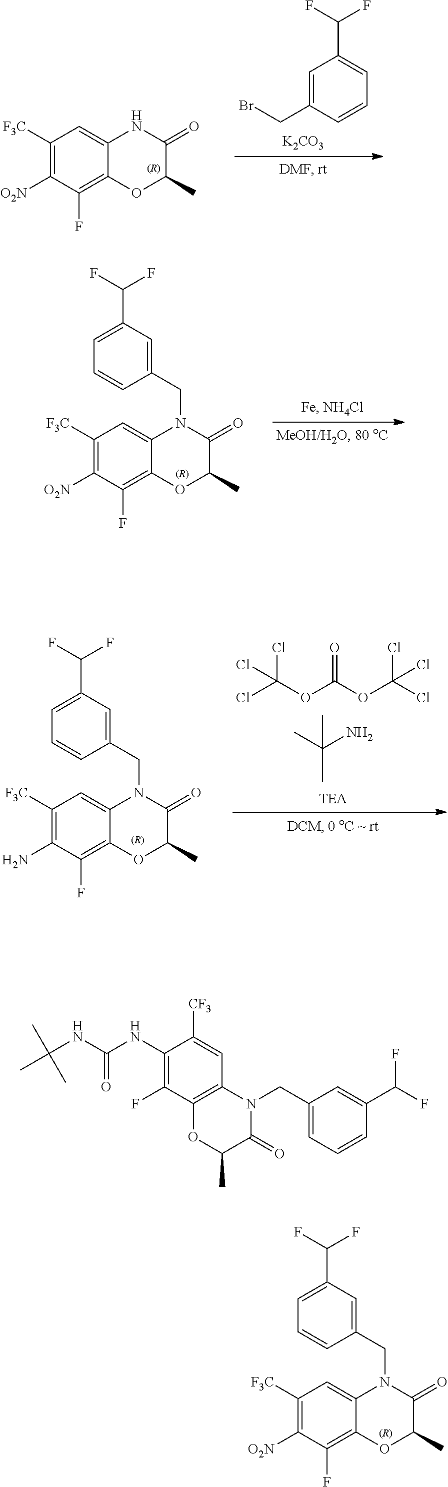

### (R)-4-(3-(difluoromethyl)benzyl)-8-fluoro-2-methyl-7-nitro-6-(trifluoromethyl)-2H-benzo[b][1,4]oxazin-3(4H)-one

To a solution of (2R)-8-fluoro-2-methyl-7-nitro-6-(trifluoromethyl)-2,4-dihydro-1,4-benzoxazin-3-one (100 mg, 0.340 mmol, 1 equiv) in DMF (10 mL) was added 1-(bromomethyl)-3-(difluoromethyl)benzene (112.72 mg, 0.510 mmol, 1.5 equiv) and K₂CO₃ (141.98 mg, 1.020 mmol, 3 equiv). The reaction was stirred at rt for 1 h. The reaction was monitored by TLC. Quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give (2R)-4-{[3-(difluoromethyl)phenyl]methyl}-8-fluoro-2-methyl-7-nitro-6-(trifluoromethyl)-2H-1,4-benzoxazin-3-one (140 mg, 94.83%) as a yellow solid.

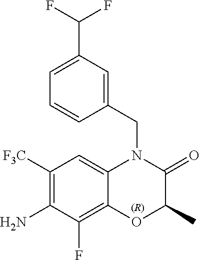

### (R)-7-amino-4-(3-(difluoromethyl)benzyl)-8-fluoro-2-methyl-6-(trifluoromethyl)-2H-benzo[b][1,4]oxazin-3(4H)-one

To a solution of (2R)-4-{[3-(difluoromethyl)phenyl]methyl}-8-fluoro-2-methyl-7-nitro-6-(trifluoromethyl)-2H-1,4-benzoxazin-3-one (130 mg, 0.299 mmol, 1 equiv) in MeOH (5 mL) and H₂O (0.5 mL) was added Fe (167.16 mg, 2.990 mmol, 10 equiv) and NH₄C₁ (160.12 mg, 2.990 mmol, 10 equiv). The reaction was stirred at 80° C. for 1 h. The mixture was allowed to cool down to rt. The resulting mixture was filtered, the filter cake was washed with MeOH (3×30 mL). The filtrate was concentrated under reduced pressure. Quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give (2R)-7-amino-4-{[3-(difluoromethyl)phenyl]methyl}-8-fluoro-2-methyl-6-(trifluoromethyl)-2H-1,4-benzoxazin-3-one (100 mg, 82.63%) as a yellow solid. LC/MS: mass calcd. for C₁₈H₁₄F₆N₂O₂:404.10 m/z, found: 405.05 [M+H]⁺.

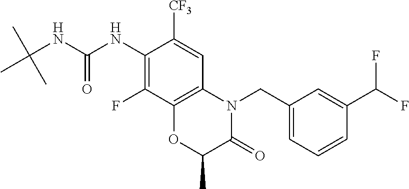 [SMILES: C[C@H]1Oc2c(cc(C(F)(F)F)c(NC(=O)NC(C)(C)C)c2F)N(Cc2cccc(C(F)F)c2)C1=O]

### (R)-1-(tert-butyl)-3-(4-(3-(difluoromethyl)benzyl)-8-fluoro-2-methyl-3-oxo-6-(trifluoromethyl)-3,4-dihydro-2H-benzo[b][1,4]oxazin-7-yl)urea

To a solution of (2R)-7-amino-4-{[3-(difluoromethyl)phenyl]methyl}-8-fluoro-2-methyl-6-(trifluoromethyl)-2H-1,4-benzoxazin-3-one (150 mg, 0.371 mmol, 1 equiv) in DCM (5 mL) was added triphosgene (110.09 mg, 0.371 mmol, 1 equiv) under N₂ at 0° C. The reaction was stirred at 0° C. under N₂ for 1 h. To the above mixture was added TEA (112.63 mg, 1.113 mmol, 3 equiv) at 0° C. The resulting mixture was stirred for additional 1 h at rt. Then, to the above mixture was added erbumine (81.40 mg, 1.113 mmol, 3 equiv). The resulting mixture was stirred for additional 1 h at rt. Quenched with water (10 mL) and extracted with DCM (3×10 mL). The combined extracts were washed with water, brine, dried over Na₂SO₄, filtered and concentrated. The residue was purified by preparative HPLC using a XBridge Prep C18 OBD Column, 19*250 mm, 5 μm column (eluent: 49% to 79% (v/v) CH₃CN and Water (10 mmol/L NH₄HCO₃+0.1% NH₃·H₂O)) to afford the title compound 3-tert-butyl-1-[(2R)-4-{[3-(difluoromethyl)phenyl]methyl}-8-fluoro-2-methyl-3-oxo-6-(trifluoromethyl)-2H-1,4-benzoxazin-7-yl]urea (20.3 mg, 10.79%) as a white solid. LC/MS: mass calcd. for C₂₃H₂₃F₆N₃O₃:503.16 m/z, found: 504.20 [M+H]⁺. ¹H NMR (400 MHz, DMSO-d₆) δ 7.46 (s, 1H), 7.31-7.40 (m, 2H), 7.23-7.31 (m, 3H), 7.06 (d, J=1.6 Hz, 1H), 6.27 (s, 1H), 5.28 (d, J=16.4 Hz, 1H), 5.21 (d, J=16.4 Hz, 1H), 5.14 (q, J=6.7 Hz, 1H), 1.59 (d, J=6.7 Hz, 3H), 1.25 (s, 9H). ¹⁹F NMR (376 MHz, DMSO) δ(ppm): −59.0147, −136.7360.

### Example 351: (S)-1-(tert-butyl)-3-(5-(hydroxymethyl)-2-oxo-1-(1-(3-(trifluoromethoxy)phenyl)ethyl)-1,2-dihydroquinoxalin-6-yl)urea

### Synthetic Scheme

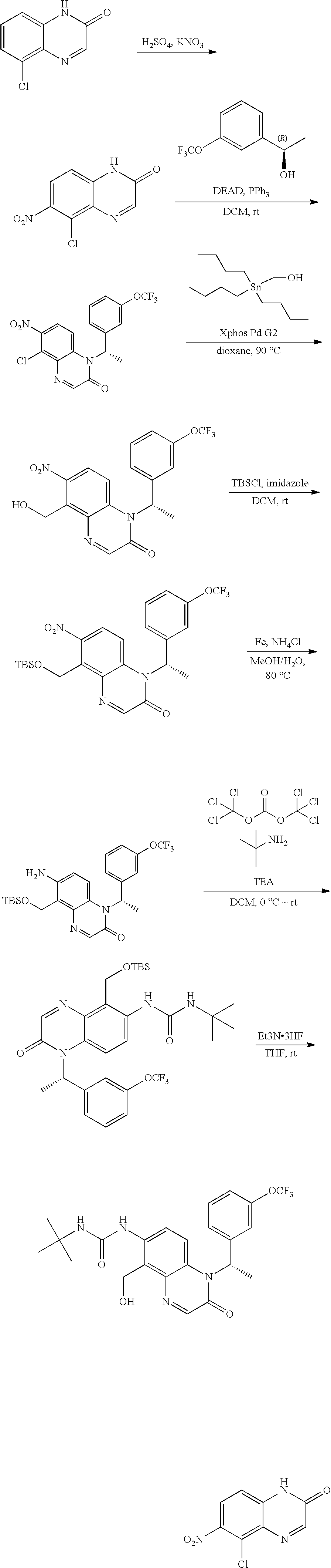

### 5-chloro-6-nitroquinoxalin-2(1H)-one

To a solution of 5-chloro-1H-quinoxalin-2-one (1 g, 5.537 mmol, 1 equiv) in H₂SO₄ (20 mL) was added KNO₃ (0.56 g, 5.537 mmol, 1 equiv) at 0° C. The reaction was stirred at rt for 3 h. The reaction was quenched by the addition of water/Ice at 0° C. Diluted with water (200 mL) and extracted with EA (3×200 mL). The combined extracts were washed with water, brine, dried over Na₂SO₄, filtered and concentrated. The residue was purified by column chromatography on silica gel with EA/PE (0-100%) to give 5-chloro-6-nitro-1H-quinoxalin-2-one (610 mg, 48.83%) as a yellow solid. LC/MS: mass calcd. for C₈H₄ClN₃O₃:224.99 m/z, found: 226.15 [M+H]⁺.

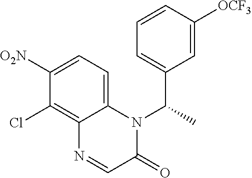 [SMILES: C[C@@H](c1cccc(OC(F)(F)F)c1)n1c(=O)cnc2c(Cl)c([N+](=O)[O-])ccc21]

### (S)-5-chloro-6-nitro-1-(1-(3-(trifluoromethoxy)phenyl)ethyl)quinoxalin-2(1H)-one

To a solution of 5-chloro-6-nitro-1H-quinoxalin-2-one (700 mg, 3.103 mmol, 1 equiv) in DCM (10 mL) was added (1R)-1-[3-(trifluoromethoxy)phenyl]ethanol (639.72 mg, 3.103 mmol, 1 equiv) and PPh₃ (1220.83 mg, 4.655 mmol, 1.5 equiv). The reaction was stirred at rt under N₂ for 0.5 h. To the above mixture was added DEAD (810.60 mg, 4.655 mmol, 1.5 equiv) at 0° C. The resulting mixture was stirred for additional 1 h at rt. Quenched with water (80 mL) and extracted with DCM (3×80 mL). The combined extracts were washed with water, brine, dried over Na₂SO₄, filtered and concentrated. The residue was purified by column chromatography on silica gel with EA/PE (0-100%) to give 5-chloro-6-nitro-1-[(1S)-1-[3-(trifluoromethoxy)phenyl]ethyl]quinoxalin-2-one (240 mg, 18.69%) as a yellow solid. LC/MS: mass calcd. for C₁₇H₁₁ClF₃N₃O₄:413.04 m/z, found: 414.05 [M+H]⁺.

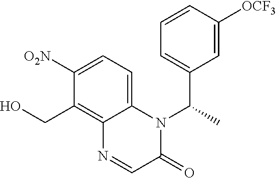 [SMILES: C[C@@H](c1cccc(OC(F)(F)F)c1)n1c(=O)cnc2c(CO)c([N+](=O)[O-])ccc21]

### (S)-5-(hydroxymethyl)-6-nitro-1-(1-(3-(trifluoromethoxy)phenyl)ethyl)quinoxalin-2(1H)-one

To a solution of 5-chloro-6-nitro-1-[(1S)-1-[3-(trifluoromethoxy)phenyl]ethyl]quinoxalin-2-one (160 mg, 0.387 mmol, 1 equiv) in dioxane (5 mL) was added X-phos Pd G2 (30.43 mg, 0.039 mmol, 0.1 equiv) and (tributylstannyl)methanol (248.34 mg, 0.774 mmol, 2 equiv). The reaction was stirred at 90° C. under N₂ for 1 h. After cooling down to rt, the reaction was quenched with water (30 mL) and extracted with EA (3×30 mL). The combined extracts were washed with water, brine, dried over Na₂SO₄, filtered and concentrated. The residue was purified by column chromatography on silica gel with EA/PE (0-100%) to give 5-(hydroxymethyl)-6-nitro-1-[(1S)-1-[3-(trifluoromethoxy)phenyl]ethyl]quinoxalin-2-one (70 mg, 44.22%) as a yellow solid. LC/MS: mass calcd. for C₁₈H₁₄F₃N₃O₅:409.09 m/z, found: 410.00 [M+H]⁺.

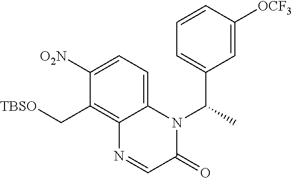 [SMILES: C[C@@H](c1cccc(OC(F)(F)F)c1)n1c(=O)cnc2c(CO[Si](C)(C)C(C)(C)C)c([N+](=O)[O-])ccc21]

### (S)-5-(((tert-butyldimethylsilyl)oxy)methyl)-6-nitro-1-(1-(3-(trifluoromethoxy)phenyl)ethyl)quinoxalin-2(1H)-one

To a solution of 5-(hydroxymethyl)-6-nitro-1-[(1S)-1-[3-(trifluoromethoxy)phenyl]ethyl]quinoxalin-2-one (45 mg, 0.110 mmol, 1 equiv) in DCM (2 mL) was added Imidazole (22.45 mg, 0.330 mmol, 3 equiv) and TBSCl (24.85 mg, 0.165 mmol, 1.5 equiv) at 0° C. The reaction was stirred at rt for 3 h. Quenched with water (20 mL) and extracted with DCM (3×20 mL). The combined extracts were washed with water, brine, dried over Na₂SO₄, filtered and concentrated. The residue was purified by column chromatography on silica gel with EA/PE (0-100%) to give 5-{[(tert-butyldimethylsilyl)oxy]methyl}-6-nitro-1-[(1S)-1-[3-(trifluoromethoxy)phenyl]ethyl]quinoxalin-2-one (45 mg, 78.18%) as a yellow solid. LC/MS: mass calcd. for C₂₄H₂₈F₃N₃O₅Si: 523.18 m/z, found: 524.15 [M+H]⁺.

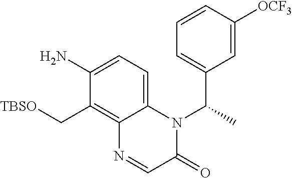 [SMILES: C[C@@H](c1cccc(OC(F)(F)F)c1)n1c(=O)cnc2c(CO[Si](C)(C)C(C)(C)C)c(N)ccc21]

### (S)-6-amino-5-(((tert-butyldimethylsilyl)oxy)methyl)-1-(1-(3-(trifluoromethoxy)phenyl)ethyl)quinoxalin-2(1H)-one

To a solution of 5-{[(tert-butyldimethylsilyl)oxy]methyl}-6-nitro-1-[(1S)-1-[3-(trifluoromethoxy)phenyl]ethyl]quinoxalin-2-one (30 mg, 0.057 mmol, 1 equiv) in MeOH (4 mL) and H₂O (0.4 mL) was added Fe (32.00 mg, 0.570 mmol, 10 equiv) and NH₄C₁ (30.65 mg, 0.570 mmol, 10 equiv). The reaction was stirred at 80° C. for 1 h. The mixture was allowed to cool down to rt. The resulting mixture was filtered, the filter cake was washed with MeOH (3×30 mL). The filtrate was concentrated under reduced pressure. Diluted with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give 6-amino-5-{[(tert-butyldimethylsilyl)oxy]methyl}-1-[(1S)-1-[3-(trifluoromethoxy)phenyl]ethyl]quinoxalin-2-one (25 mg, 88.40%) as a yellow solid. LC/MS: mass calcd. for C₂₄H₃₀F₃N₃O₃Si: 493.20 m/z, found: 494.15 [M+H]⁺.

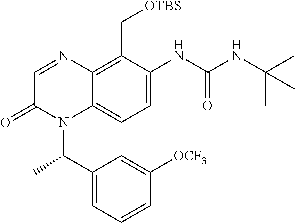 [SMILES: C[C@@H](c1cccc(OC(F)(F)F)c1)n1c(=O)cnc2c(CO[Si](C)(C)C(C)(C)C)c(NC(=O)NC(C)(C)C)ccc21]

### (S)-1-(tert-butyl)-3-(5-(((tert-butyldimethylsilyl)oxy)methyl)-2-oxo-1-(1-(3-(trifluoromethoxy)phenyl)ethyl)-1,2-dihydroquinoxalin-6-yl)urea

To a solution of 6-amino-5-{[(tert-butyldimethylsilyl)oxy]methyl}-1-[(1S)-1-[3-(trifluoromethoxy)phenyl]ethyl]quinoxalin-2-one (20 mg, 0.041 mmol, 1 equiv) in DCM (5 mL) was added triphosgene (12.02 mg, 0.041 mmol, 1 equiv) under N₂ at 0° C. The reaction was stirred at 0° C. under N₂ for 1 h. To the above mixture was added TEA (12.30 mg, 0.123 mmol, 3 equiv) at 0° C. The resulting mixture was stirred for additional 1 h at rt. Then, to the above mixture was added erbumine (8.89 mg, 0.123 mmol, 3 equiv). The resulting mixture was stirred for additional 1 h at rt. Quenched with water (20 mL) and extracted with DCM (3×20 mL). The combined extracts were washed with water, brine, dried over Na₂SO₄, filtered and concentrated. The residue was purified by column chromatography on silica gel with EA/PE (0-100%) to give 3-tert-butyl-1-(5-{[(tert-butyldimethylsilyl)oxy]methyl}-2-oxo-1-[(1S)-1-[3-(trifluoromethoxy)phenyl]ethyl]quinoxalin-6-yl)urea (20 mg, 83.28%) as a yellow solid. LC/MS: mass calcd. for C₂₉H₃₉F₃N₄O₄Si: 592.27 m/z, found: 615.10 [M+Na]⁺.

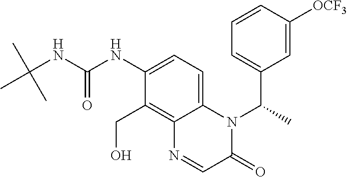 [SMILES: C[C@@H](c1cccc(OC(F)(F)F)c1)n1c(=O)cnc2c(CO)c(NC(=O)NC(C)(C)C)ccc21]

### (S)-1-(tert-butyl)-3-(5-(hydroxymethyl)-2-oxo-1-(1-(3-(trifluoromethoxy)phenyl)ethyl)-1,2-dihydroquinoxalin-6-yl)urea

To a solution of 3-tert-butyl-1-(5-{[(tert-butyldimethylsilyl)oxy]methyl}-2-oxo-1-[(1S)-1-[3-(trifluoromethoxy)phenyl]ethyl]quinoxalin-6-yl)urea (20 mg, 0.034 mmol, 1 equiv) in THF (1 mL) was added Et₃N·3HF (8.16 mg, 0.051 mmol, 1.5 equiv) at 0° C. The reaction was stirred at rt for 1 h. Quenched with water (20 mL) and extracted with EA (3×20 mL). The combined extracts were washed with water, brine, dried over Na₂SO₄, filtered and concentrated. The residue was purified by preparative HPLC using a YMC Triart C18 ExRs Column, 20*250 mm, 5 μm column (eluent: 45% to 75% (v/v) CH₃CN and Water (10 mmol/L NH₄HCO₃+0.1% NH₃·H₂O)) to afford the title compound 3-tert-butyl-1-[5-(hydroxymethyl)-2-oxo-1-[(1S)-1-[3-(trifluoromethoxy)phenyl]ethyl]quinoxalin-6-yl]urea (9.3 mg, 57.33%) as a white solid. LC/MS: mass calcd. for C₂₃H₂₅F₃N₄O₄:478.18 m/z, found: 477.15 [M−H]⁻. ¹H NMR (400 MHz, DMSO-d₆) δ 8.30 (s, 1H), 7.90-8.10 (m, 2H), 7.47 (dd, J=9.2, 7.2 Hz, 1H), 7.24-7.32 (m, 3H), 6.90 (s, 1H), 6.62 (s, 1H), 5.17 (s, 1H), 5.04 (dd, J=11.7, 4.9 Hz, 1H), 4.96 (dd, J=11.8, 5.0 Hz, 1H), 1.90 (d, J=7.1 Hz, 3H), 1.28 (s, 9H). ¹⁹F NMR (376 MHz, DMSO) δ(ppm): −56.6537.

### Example 352: 3-tert-butyl-1-{7-cyano-2-oxo-1-[(1S)-1-[3-(trifluoromethoxy)phenyl]ethyl]quinoxalin-6-yl}urea

### Synthetic Scheme

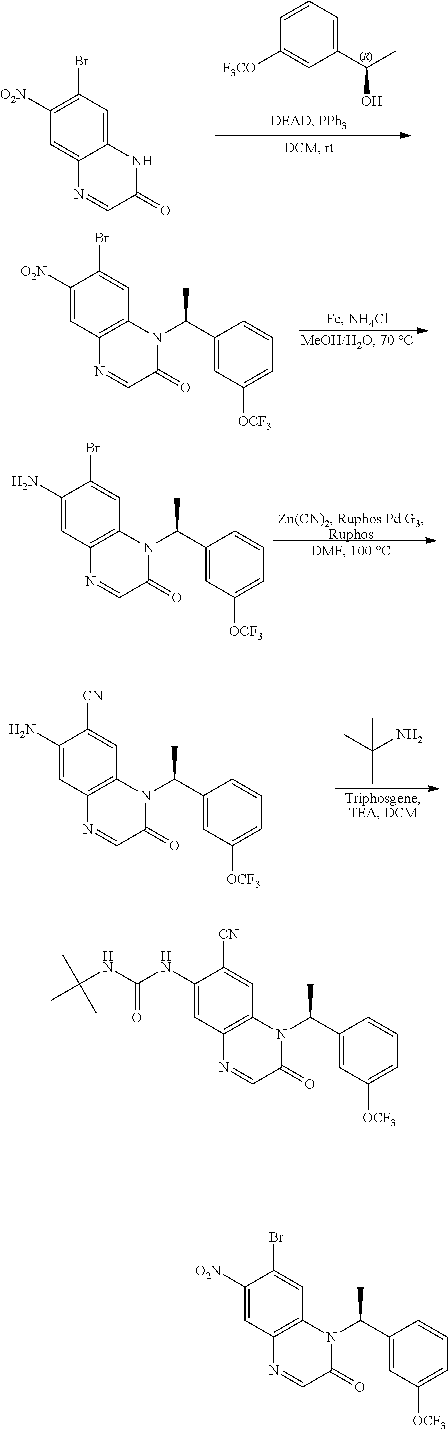

### 7-bromo-6-nitro-1-[(1S)-1-[3-(trifluoromethoxy)phenyl]ethyl]quinoxalin-2-one

To a solution of 7-bromo-6-nitro-1H-quinoxalin-2-one (1 g, 3.703 mmol, 1 equiv) in DCM (15 mL) was added (2R,4E)-3-methylidene-5-(trifluoromethoxy)hex-4-en-2-ol (0.73 g, 3.703 mmol, 1 equiv) and PPh₃ (1.46 g, 5.554 mmol, 1.5 equiv). After the mixture was stirred at rt for 1 h, to the above mixture was added DEAD (0.64 g, 3.703 mmol, 1 equiv) dropwise at 0° C. The resulting mixture was stirred for additional for 2 h at 25° C. Quenched with water (100 mL) and extracted with DCM (3×100 mL). The combined extracts were washed with water, brine, dried over Na₂SO₄, filtered and concentrated. The residue was purified by column chromatography on silica gel with EA/PE (0-100%) to give 7-bromo-6-nitro-1-[(1S)-1-[3-(trifluoromethoxy)phenyl]ethyl]quinoxalin-2-one (130 mg, 7.66%) as a yellow oil. MS (ESI): mass calcd. for C₁₇H₁₁BrF₃N₃O₄:456.99 m/z, found: 457.99, 459.99 [M+H, M+H+2]⁺.

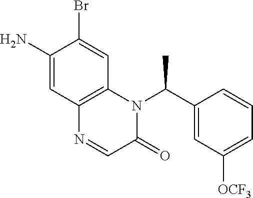 [SMILES: C[C@@H](c1cccc(OC(F)(F)F)c1)n1c(=O)cnc2cc(N)c(Br)cc21]

### 6-amino-7-bromo-1-[(1S)-1-[3-(trifluoromethoxy)phenyl]ethyl]quinoxalin-2-one

To a solution of 7-bromo-6-nitro-1-[(1S)-1-[3-(trifluoromethoxy)phenyl]ethyl]quinoxalin-2-one (110 mg, 0.240 mmol, 1 equiv) in MeOH (1 mL) and H₂O (10 mL) was added Fe (134.07 mg, 2.400 mmol, 10 equiv) and NH₄C₁ (128.42 mg, 2.400 mmol, 10 equiv). The reaction was stirred at 70° C. for 2 h. The mixture was allowed to cool down to room temperature and quenched with water (50 mL). The resulting mixture was filtered; the filter cake was washed with EA (50 mL). The filtrate was extracted with EA (3×50 mL). The combined extracts were washed with water, brine, dried over Na₂SO₄, filtered and concentrated. The residue was purified by column chromatography on silica gel with EA/PE (0-100%) to give 6-amino-7-bromo-1-[(1S)-1-[3-(trifluoromethoxy)phenyl]ethyl]quinoxalin-2-one (50 mg, 48.64%) as a yellow solid. MS (ESI): mass calcd. for C₁₇H₁₃BrF₃N₃O₂:427.01 m/z, found: 428.01, 430.01 [M+H, M+H+2]⁺.

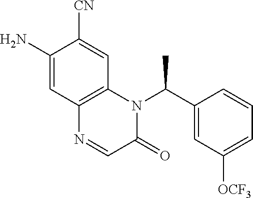 [SMILES: C[C@@H](c1cccc(OC(F)(F)F)c1)n1c(=O)cnc2cc(N)c(C#N)cc21]

### 7-amino-3-oxo-4-[(1S)-1-[3-(trifluoromethoxy)phenyl]ethyl]quinoxaline-6-carbonitrile

To a solution of 6-amino-7-bromo-1-[(1S)-1-[3-(trifluoromethoxy)phenyl]ethyl]quinoxalin-2-one (60 mg, 0.140 mmol, 1 equiv) in dimethylformamide (4 mL) was added Zn(CN)₂ (19.74 mg, 0.168 mmol, 1.2 equiv) and Ruphos (6.54 mg, 0.014 mmol, 0.1 equiv) RuPhos Pd G3 (11.72 mg, 0.014 mmol, 0.1 equiv). The reaction was stirred at 100° C. for 2 h under N₂ atmosphere. The mixture was allowed to cool down to room temperature. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give 7-amino-3-oxo-4-[(1S)-1-[3-(trifluoromethoxy)phenyl]ethyl]quinoxaline-6-carbonitrile (15 mg, 28.60%) as a yellow oil. MS (ESI): mass calcd. for C₁₈H₁₃F₃N₄O₂:374.10 m/z, found: 375.10 [M+H]⁺.

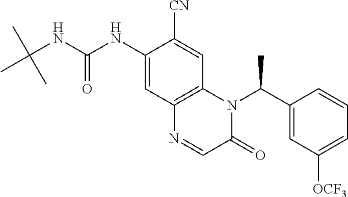 [SMILES: C[C@@H](c1cccc(OC(F)(F)F)c1)n1c(=O)cnc2cc(NC(=O)NC(C)(C)C)c(C#N)cc21]

### 3-tert-butyl-1-{7-cyano-2-oxo-1-[(1S)-1-[3-(trifluoromethoxy)phenyl]ethyl]quinoxalin-6-yl}urea

To a solution of 7-amino-3-oxo-4-[(1S)-1-[3-(trifluoromethoxy)phenyl]ethyl]quinoxaline-6-carbonitrile (15 mg, 0.040 mmol, 1 equiv) in DCM (2 mL) was added triphosgene (5.95 mg, 0.020 mmol, 0.5 equiv) at 0° C. under N₂ atmosphere. The resulting mixture was stirred 2 h at 25° C. Then TEA (16.22 mg, 0.159 mmol, 3 equiv) was added into above system at 0° C. The resulting mixture was stirred 1 h at 25° C. To the above mixture were added erbumine (4.40 mg, 0.060 mmol, 1.5 equiv) dropwise at 0° C. The reaction was stirred at 25° C. for 2 h. Quenched with water (30 mL) and extracted with DCM (3×20 mL). The combined extracts were washed with water, brine, dried over Na₂SO₄, filtered and concentrated. The residue was purified by preparative HPLC using a XBridge Prep OBD 19*250 mm, 5 μm column (eluent: 45% to 75% (v/v) CH₃CN and H₂O with 10 mmol/L NH₄HCO₃) to afford the title compound 3-tert-butyl-1-{7-cyano-2-oxo-1-[(1S)-1-[3-(trifluoromethoxy)phenyl]ethyl]quinoxalin-6-yl}urea (4.1 mg, 21.08%) as a yellow solid. LC/MS (ESI): mass calcd. for C₂₃H₂₂F₃N₅O₃:473.17 m/z, found: 474.15 [M+H]⁺. ¹H NMR (400 MHz, DMSO-d₆) δ 8.50 (s, 1H), 8.39 (s, 1H), 8.33 (s, 1H), 7.44-7.72 (m, 2H), 7.27-7.38 (m, 3H), 6.95 (s, 1H), 6.43 (m, 1H), 1.88 (d, J=7.0 Hz, 3H), 1.32 (s, 9H). ¹⁹F NMR (376 MHz, DMSO) δ −56.71.

### Example 353: 3-tert-butyl-1-{5-fluoro-2-oxo-1-[(1S)-1-[3-(trifluoromethoxy)phenyl]ethyl]quinoxalin-6-yl}urea

### Synthetic Scheme

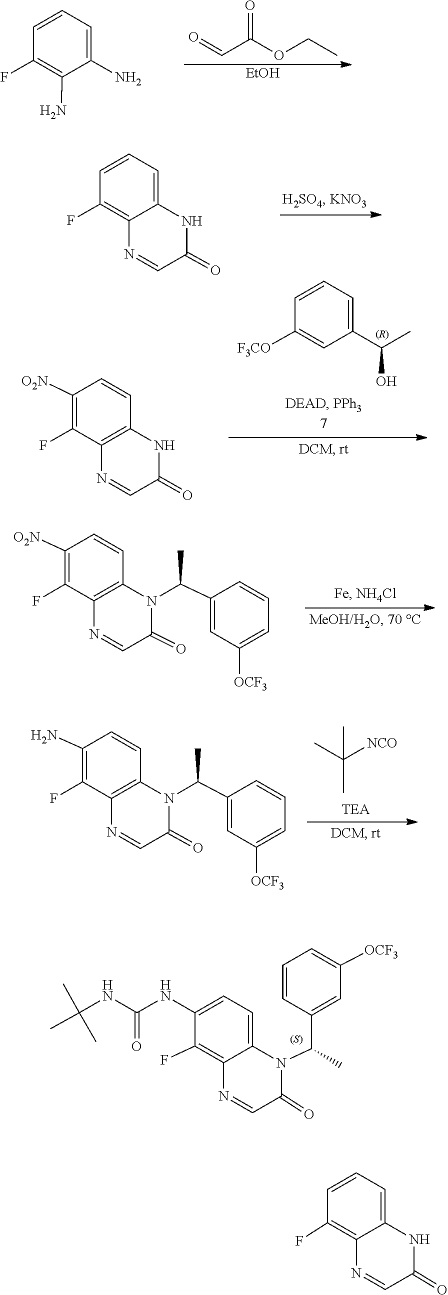

### 5-fluoro-1H-quinoxalin-2-one

To a stirred solution of 3-fluorobenzene-1,2-diamine (2 g, 15.856 mmol, 1 equiv) in EtOH (20 mL) was added ethyl glyoxylate (1.62 g, 15.856 mmol, 1 equiv) in portions at room temperature under air atmosphere. The resulting mixture was stirred for 3 h at room temperature under air atmosphere. The reaction was monitored by LCMS. Quenched with water (100 mL) and extracted with EA (3×100 mL). The combined extracts were washed with water, brine, dried over Na₂SO₄, filtered and concentrated. The residue was purified by silica gel column chromatography, eluted with PE/EA (0-100%) to afford 5-fluoro-1H-quinoxalin-2-one (1 g, 38.4%) as a brown solid. LC/MS (ESI): mass calcd. for C₈H₅FN₂O: 164.04 m/z, found: 165.0 [M+H]⁺.

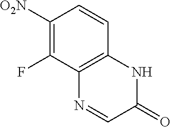 [SMILES: O=c1cnc2c(F)c([N+](=O)[O-])ccc2[nH]1]

### 5-fluoro-6-nitro-1H-quinoxalin-2-one

To a stirred solution of 5-fluoro-1H-quinoxalin-2-one (1 g, 6.092 mmol, 1 equiv) in H₂SO₄ (10 mL) was added KNO₃ (615.95 mg, 6.092 mmol, 1 equiv) in portions at 0° C. under air atmosphere. The resulting mixture was stirred for 2 h at room temperature under air atmosphere. The reaction was monitored by LCMS. The reaction was quenched with water/Ice (50 mL) at 0° C., And extracted with EA (3×50 mL). The combined extracts were washed with water, brine, dried over Na₂SO₄, filtered and concentrated. The residue was purified by silica gel column chromatography, eluted with PE/EA (0-100%) to afford 5-fluoro-6-nitro-1H-quinoxalin-2-one (400 mg, 31.39%) as a brown solid. LC/MS (ESI): mass calcd. for C₈H₄FN₃O₃:209.0 m/z, found: 210.0 [M+H]⁺.

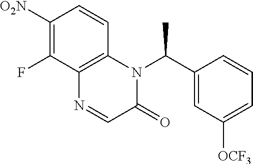 [SMILES: C[C@@H](c1cccc(OC(F)(F)F)c1)n1c(=O)cnc2c(F)c([N+](=O)[O-])ccc21]

### 5-fluoro-6-nitro-1-[(1S)-1-[3-(trifluoromethoxy)phenyl]ethyl]quinoxalin-2-one

To a stirred solution of 5-fluoro-6-nitro-1H-quinoxalin-2-one (400 mg, 1.913 mmol, 1 equiv) and (1R)-1-[3-(trifluoromethoxy)phenyl]ethanol (394.32 mg, 1.913 mmol, 1 equiv) in DCM (4 mL) was added PPh₃ (752.50 mg, 2.869 mmol, 1.5 equiv) in portions at rt under nitrogen atmosphere. The resulting mixture was stirred for 30 min at room temperature under nitrogen atmosphere. To the above mixture was added DEAD (333.10 mg, 1.913 mmol, 1 equiv) in portions at 0° C. The resulting mixture was stirred for 2 h at room temperature under nitrogen atmosphere. The reaction was monitored by LCMS. Quenched with water (30 mL) and extracted with EA (3×30 mL). The combined extracts were washed with water, brine, dried over Na₂SO₄, filtered and concentrated. The residue was purified by silica gel column chromatography, eluted with PE/EA (0-100%) to afford 5-fluoro-6-nitro-1-[(1S)-1-[3-(trifluoromethoxy)phenyl]ethyl]quinoxalin-2-one (80 mg, 10.53%) as a yellow solid. LC/MS (ESI): mass calcd. for C₁₇H₁₁F₄N₃O₄:397.1 m/z, found: 398.0 [M+H]⁺.

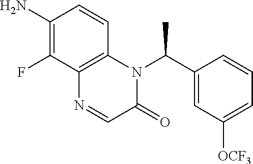 [SMILES: C[C@@H](c1cccc(OC(F)(F)F)c1)n1c(=O)cnc2c(F)c(N)ccc21]

### 6-amino-5-fluoro-1-[(1S)-1-[3-(trifluoromethoxy)phenyl]ethyl]quinoxalin-2-one

To a stirred solution of 5-fluoro-6-nitro-1-[(1S)-1-[3-(trifluoromethoxy)phenyl]ethyl]quinoxalin-2-one (80 mg, 0.201 mmol, 1 equiv) in MeOH/H₂O (10:1, 1.1 mL) were added NH₄C₁ (107.71 mg, 2.010 mmol, 10 equiv) and Fe (14.06 mg, 0.250 mmol, 10 equiv) in portions at room temperature under air atmosphere. The resulting mixture was stirred for 2 h at 70° C. under air atmosphere. The reaction was monitored by LCMS. The mixture was allowed to cool down to room temperature. Quenched with water (10 mL) and extracted with EA (3×10 mL). The combined extracts were washed with water, brine, dried over Na₂SO₄, filtered and concentrated. The residue was purified by silica gel column chromatography, eluted with PE/EA (0-100%) to afford 6-amino-5-fluoro-1-[(1S)-1-[3-(trifluoromethoxy)phenyl]ethyl]quinoxalin-2-one (30 mg, 40.56%) as a yellow solid. LC/MS (ESI): mass calcd. for C₁₇H₁₃F₄N₃O₂:367.1 m/z, found: 367.95 [M+H]⁺.

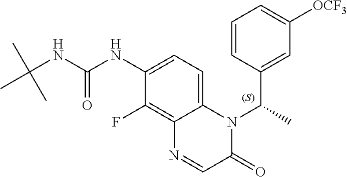

### 3-tert-butyl-1-{5-fluoro-2-oxo-1-[(1S)-1-[3-(trifluoromethoxy)phenyl]ethyl]quinoxalin-6-yl}urea

To a stirred solution of 6-amino-5-fluoro-1-[(1S)-1-[3-(trifluoromethoxy)phenyl]ethyl]quinoxalin-2-one (20 mg, 0.054 mmol, 1 equiv) in DCM (1 mL) were added 2-isocyanato-2-methylpropane (16.19 mg, 0.162 mmol, 3 equiv) and TEA (16.53 mg, 0.162 mmol, 3 equiv) in portions at room temperature under air atmosphere. The resulting mixture was stirred for 1 days at room temperature under air atmosphere. The reaction was monitored by LCMS. Quenched with water (5 mL) and extracted with DCM (3×5 mL). The combined extracts were washed with water, brine, dried over Na₂SO₄, filtered and concentrated. The residue was purified by preparative HPLC using a X Bridge Prep OBD C18 250 mm×19 mm×5 μm column (eluent: 45% to 75% (v/v) CH₃CN and H₂O with 10 mmol/L NH₄HCO₃0.1% NH₃·H₂O) to afford 3-tert-butyl-1-{5-fluoro-2-oxo-1-[(1S)-1-[3-(trifluoromethoxy)phenyl]ethyl]quinoxalin-6-yl}urea (2.3 mg, 9.03%) as a yellow solid. LC/MS (ESI): mass calcd. for C₂₂H₂₂F₄N₄O₃:466.2 m/z, found: 467.2 [M+H]⁺. ¹H NMR (300 MHz, Chloroform-d) δ 8.17 (s, 1H), 7.65 (d, J=15.3 Hz, 1H), 7.26-7.23 (m, 2H), 7.03-6.98 (m, 3H), 6.50 (s, 1H), 6.19 (s, 1H), 1.88-1.85 (m, 3H), 1.32 (s, 9H). ¹⁹F NMR (282 MHz, Chloroform-d) δ −57.75.

### Example 354: (R)-1-(4-benzyl-8-fluoro-2-methyl-3-oxo-6-(trifluoromethyl)-3,4-dihydro-2H-benzo[b][1,4]oxazin-7-yl)-3-(tert-butyl)urea

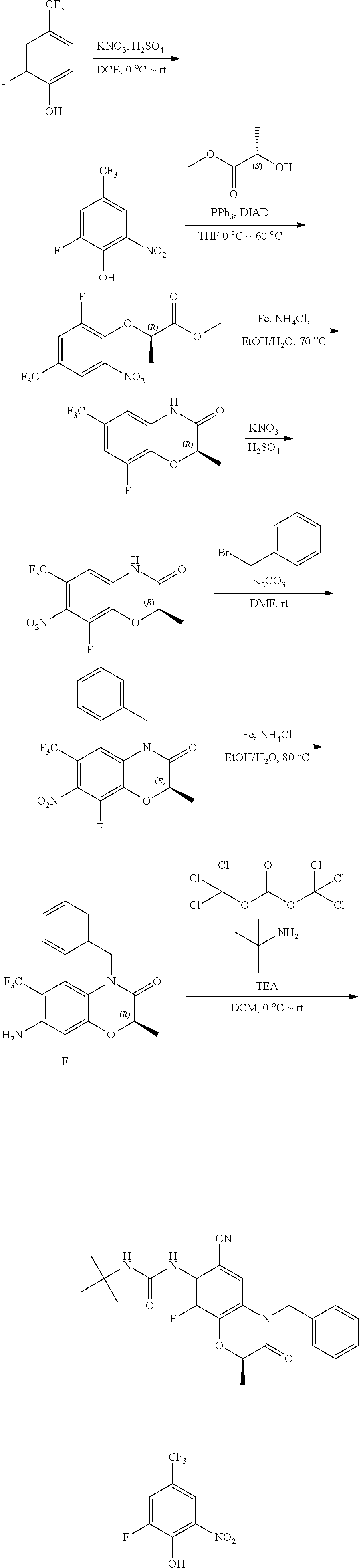

### 2-fluoro-6-nitro-4-(trifluoromethyl)phenol

To a solution of 2-fluoro-4-(trifluoromethyl)phenol (2 g, 11.105 mmol, 1 equiv) in DCE (20 mL) was added H₂SO₄ (20 mL) at −10° C. And to the above mixture was added KNO₃ (1.12 g, 11.105 mmol, 1 equiv) at −10° C. The resulting mixture was stirred for additional 30 min at −10° C. The reaction was quenched by the addition of water/Ice at 0° C. Quenched with water (400 mL) and extracted with EA (3×400 mL). The combined extracts were washed with water, brine, dried over Na₂SO₄, filtered and concentrated. This resulted in 2-fluoro-6-nitro-4-(trifluoromethyl)phenol (2.14 g, 85.61%) as a yellow oil. LC/MS: mass calcd. for C₇H₃F₄NO₃:225.00 m/z, found: 223.95 [M−H]⁻.

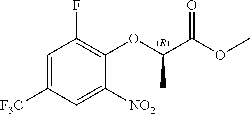

### methyl (R)-2-(2-fluoro-6-nitro-4-(trifluoromethyl)phenoxy)propanoate

To a solution of 2-fluoro-6-nitro-4-(trifluoromethyl)phenol (1.4 g, 6.219 mmol, 1 equiv) in THF (10 mL) was added methyl (2S)-2-hydroxypropanoate (647.48 mg, 6.219 mmol, 1 equiv) and PPh₃ (2446.98 mg, 9.329 mmol, 1.5 equiv). The reaction was stirred at rt under N₂ for 0.5 h. To the above mixture was added DIAD (1886.46 mg, 9.329 mmol, 1.5 equiv) at 0° C. The resulting mixture was stirred for additional 1 h at 60° C. After cooling down to rt, the reaction was quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give methyl (2R)-2-[2-fluoro-6-nitro-4-(trifluoromethyl)phenoxy]propanoate (1.14 g, 58.90%) as a yellow oil. LC/MS: mass calcd. for C₁₁H₉F₄NO₅:311.04 m/z, found: 311.90 [M+H]⁺.

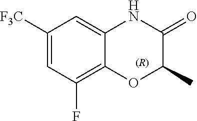

### (R)-8-fluoro-2-methyl-6-(trifluoromethyl)-2H-benzo[b][1,4]oxazin-3(4H)-one

To a solution of methyl (2R)-2-[2-fluoro-6-nitro-4-(trifluoromethyl)phenoxy]propanoate (1.14 g, 3.663 mmol, 1 equiv) in EtOH (8 mL) and H₂O (2 mL) was added Fe (2.05 g, 36.630 mmol, 10 equiv) and NH₄C₁ (1.96 g, 36.630 mmol, 10 equiv). The reaction was stirred at 70° C. for 1 h. The mixture was allowed to cool down to rt. The filtrate was concentrated under reduced pressure. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-8-fluoro-2-methyl-6-(trifluoromethyl)-2,4-dihydro-1,4-benzoxazin-3-one (720 mg, 78.88%) as a yellow solid. LC/MS: mass calcd. for C₁₀H₇F₄NO₂:249.04 m/z, found: 249.95 [M+H]⁺.

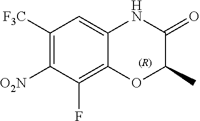

### (R)-8-fluoro-2-methyl-7-nitro-6-(trifluoromethyl)-2H-benzo[b][1,4]oxazin-3(4H)-one

To a solution of (2R)-8-fluoro-2-methyl-6-(trifluoromethyl)-2,4-dihydro-1,4-benzoxazin-3-one (700 mg, 2.809 mmol, 1 equiv) in H₂SO₄ (10 mL) was added KNO₃ (284.03 mg, 2.809 mmol, 1 equiv) at 0° C. The reaction was stirred at rt for 2 h. The reaction was quenched with water/Ice at 0° C. Diluted with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give (2R)-8-fluoro-2-methyl-7-nitro-6-(trifluoromethyl)-2,4-dihydro-1,4-benzoxazin-3-one (400 mg, 48.40%) as a yellow solid. LC/MS: mass calcd. for C₁₀H₈F₄N₂O₄:294.03 m/z, found: 292.95 [M−H]⁻.

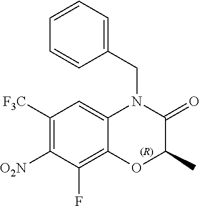

### (R)-4-benzyl-8-fluoro-2-methyl-7-nitro-6-(trifluoromethyl)-2H-benzo[b][1,4]oxazin-3(4H)-one

To a solution of (2R)-8-fluoro-2-methyl-7-nitro-6-(trifluoromethyl)-2,4-dihydro-1,4-benzoxazin-3-one (420 mg, 1.428 mmol, 1 equiv) in DMF (10 mL) was added benzyl bromide (366.31 mg, 2.142 mmol, 1.5 equiv) and K₂CO₃ (596.30 mg, 4.284 mmol, 3 equiv) at 0° C. The reaction was stirred at rt for 1 h. The reaction was monitored by TLC. Quenched with water (30 mL) and extracted with EA (3×30 mL). The combined extracts were washed with water, brine, dried over Na₂SO₄, filtered and concentrated. The residue was purified by column chromatography on silica gel with EA/PE (0-100%) to give (2R)-4-benzyl-8-fluoro-2-methyl-7-nitro-6-(trifluoromethyl)-2H-1,4-benzoxazin-3-one (400 mg, 72.90%) as a yellow solid.

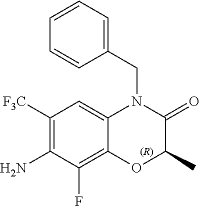

### (R)-7-amino-4-benzyl-8-fluoro-2-methyl-6-(trifluoromethyl)-2H-benzo[b][1,4]oxazin-3(4H)-one

To a solution of (2R)-4-benzyl-8-fluoro-2-methyl-7-nitro-6-(trifluoromethyl)-2H-1,4-benzoxazin-3-one (400 mg, 1.041 mmol, 1 equiv) in MeOH (10 mL) and H₂O (1 mL) was added Fe (581.28 mg, 10.410 mmol, 10 equiv) and NH₄C₁ (556.77 mg, 10.410 mmol, 10 equiv). The reaction was stirred at 80° C. for 1 h. The mixture was allowed to cool down to rt. The resulting mixture was filtered, the filter cake was washed with MeOH (3×50 mL). The filtrate was concentrated under reduced pressure. Diluted with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-7-amino-4-benzyl-8-fluoro-2-methyl-6-(trifluoromethyl)-2H-1,4-benzoxazin-3-one (300 mg, 81.35%) as a yellow solid. LC/MS: mass calcd. for C₁₇H₁₄F₄N₂O₂:354.10 m/z, found: 355.10 [M+H]⁺.

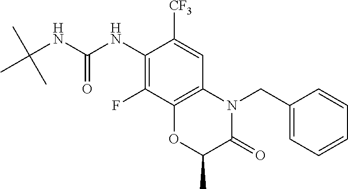 [SMILES: C[C@H]1Oc2c(cc(C(F)(F)F)c(NC(=O)NC(C)(C)C)c2F)N(Cc2ccccc2)C1=O]

### (R)-1-(4-benzyl-8-fluoro-2-methyl-3-oxo-6-(trifluoromethyl)-3,4-dihydro-2H-benzo[b][1,4]oxazin-7-yl)-3-(tert-butyl)urea

To a solution of (2R)-7-amino-4-benzyl-8-fluoro-2-methyl-6-(trifluoromethyl)-2H-1,4-benzoxazin-3-one (70 mg, 0.198 mmol, 1 equiv) in DCM (5 mL) was added triphosgene (58.62 mg, 0.198 mmol, 1 equiv) under N₂ at 0° C. The reaction was stirred at 0° C. under N₂ for 1 h. To the above mixture was added TEA (19.99 mg, 0.198 mmol, 1 equiv) at 0° C. The resulting mixture was stirred for additional 1 h at rt. Then, to the above mixture was added erbumine (14.45 mg, 0.198 mmol, 1 equiv). The resulting mixture was stirred for additional 1 h at rt. Quenched with water (20 mL) and extracted with DCM (3×20 mL). The combined extracts were washed with water, brine, dried over Na₂SO₄, filtered and concentrated. The residue was purified by preparative HPLC using a XBridge Shield RP 18 OBD Column, 19*250 mm, 5 μm column (eluent: 52% to 82% (v/v) CH₃CN and Water (10 mmol/L NH₄HCO₃+0.1% NH₃·H₂O)) to afford the title compound 1-[(2R)-4-benzyl-8-fluoro-2-methyl-3-oxo-6-(trifluoromethyl)-2H-1,4-benzoxazin-7-yl]-3-tert-butylurea (27.2 mg, 30.36%) as a white solid. LC/MS: mass calcd. for C₂₂H₂₃F₄N₃O₃:453.17 m/z, found: 454.10 [M+H]⁺. ¹H NMR (400 MHz, DMSO-d₆) δ 7.46 (s, 1H), 7.31-7.40 (m, 2H), 7.23-7.31 (m, 3H), 7.06 (d, J=1.6 Hz, 1H), 6.27 (s, 1H), 5.28 (d, J=16.4 Hz, 1H), 5.21 (d, J=16.4 Hz, 1H), 5.14 (q, J=6.7 Hz, 1H), 1.59 (d, J=6.7 Hz, 3H), 1.25 (s, 9H). ¹⁹F NMR (376 MHz, DMSO) δ(ppm): −59.0147, −136.7360.

### Example 355: 1-[(3R)-1-benzyl-7-chloro-3-methyl-2-oxo-3H-pyrido[2,3-b][1,4]oxazin-6-yl]-3-tert-butylurea

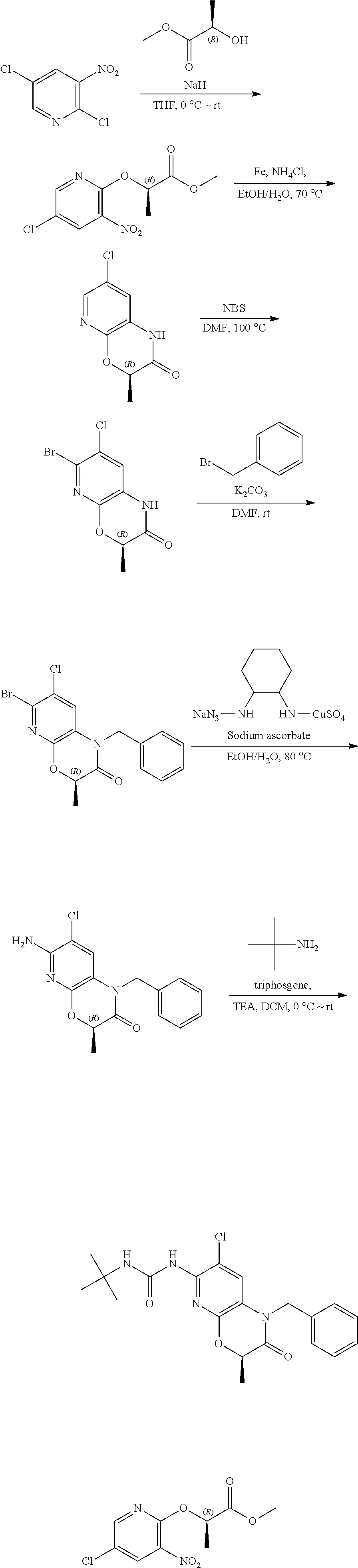

### methyl (2R)-2-[(5-chloro-3-nitropyridin-2-yl)oxy]propanoate

To a stirred solution of methyl (2R)-2-hydroxypropanoate (1.08 g, 10.364 mmol, 1 equiv) in THF (30 mL) was added NaH (0.75 g, 31.092 mmol, 3 equiv) in portions at 0° C. under air atmosphere. The resulting mixture was stirred for 30 min at room temperature under air atmosphere. To the above mixture was added 2,5-dichloro-3-nitropyridine (2 g, 10.364 mmol, 1 equiv) in portions at 0° C. The resulting mixture was stirred for additional 1 h at room temperature. The reaction was quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to afford methyl (2R)-2-[(5-chloro-3-nitropyridin-2-yl)oxy]propanoate (1 g, 37.02%) as a white solid. LC/MS (ESI): mass calcd. for C₉H₉ClN₂O₅:260.0 m/z, found: 261.10 [M+H]⁺.

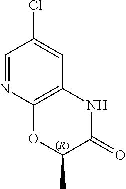

### (3R)-7-chloro-3-methyl-1H,3H-pyrido[2,3-b][1,4]oxazin-2-one

To a stirred mixture of methyl (2R)-2-[(5-chloro-3-nitropyridin-2-yl)oxy]propanoate (800 mg, 3.069 mmol, 1 equiv) and NH₄C₁ (1641.87 mg, 30.690 mmol, 10 equiv) in MeOH:H₂O=10:1 (15 mL) was added Fe (1714.15 mg, 30.690 mmol, 10 equiv) in portions at room temperature under air atmosphere. The resulting mixture was stirred for 1 h at 80° C. under air atmosphere. After cooling down to room temperature, the reaction was quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to afford (3R)-7-chloro-3-methyl-1H,3H-pyrido[2,3-b][1,4]oxazin-2-one (300 mg, 49.21%) as a yellow oil. LC/MS (ESI): mass calcd. for C₈H₇ClN₂O₂:198.0 m/z, found: 199.15 [M+H]⁺.

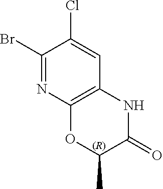

### (3R)-6-bromo-7-chloro-3-methyl-1H,3H-pyrido[2,3-b][1,4]oxazin-2-one

To a stirred solution of (3R)-7-chloro-3-methyl-1H,3H-pyrido[2,3-b][1,4]oxazin-2-one (210 mg, 1.057 mmol, 1 equiv) in DMF (5 mL) were added NBS (1129.15 mg, 6.342 mmol, 6 equiv) in portions at room temperature under air atmosphere. The resulting mixture was stirred for 4 h at 100° C. under air atmosphere. After cooling down to room temperature, the reaction was quenched with water (20 mL) and extracted with EA (3×30 mL). The combined extracts were washed with water, brine, dried over Na₂SO₄, filtered and concentrated. The residue was purified by column chromatography on silica gel with EA/PE (0-100%) to afford (3R)-6-bromo-7-chloro-3-methyl-1H,3H-pyrido[2,3-b][1,4]oxazin-2-one (170 mg, 57.94%) as a yellow solid. LC/MS (ESI): mass calcd. for C₈H₆BrClN₂O₂: 275.9 m/z, found: 274.80, 272.80 [M−H, M−H+2]⁻.

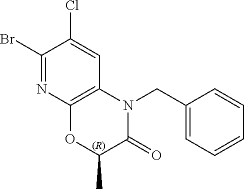

### (3R)-1-benzyl-6-bromo-7-chloro-3-methyl-3H-pyrido[2,3-b][1,4]oxazin-2-one

To a stirred mixture of (3R)-6-bromo-7-chloro-3-methyl-1H,3H-pyrido[2,3-b][1,4]oxazin-2-one (160 mg, 0.577 mmol, 1 equiv) and benzyl bromide (197.23 mg, 1.154 mmol, 2 equiv) in DMF (5 mL) was added K₂CO₃ (240.80 mg, 1.731 mmol, 3 equiv) in portions at room temperature under air atmosphere. The resulting mixture was stirred for 4 h at rt under air atmosphere. After cooling down to room temperature, the reaction was quenched with water (20 mL) and extracted with EA (3×20 mL). The combined extracts were washed with water, brine, dried over Na₂SO₄, filtered and concentrated. Desired product could be detected by LCMS. The residue was purified by column chromatography on silica gel with EA/PE (0-100%) to afford (3R)-1-benzyl-6-bromo-7-chloro-3-methyl-3H-pyrido[2,3-b][1,4]oxazin-2-one (127 mg, 59.92%) as a yellow solid. LC/MS (ESI): mass calcd. for C₁₅H₁₂BrClN₂O₂:366.0 m/z, found: 367.0, 369.0 [M+H, M+H+2]⁺.

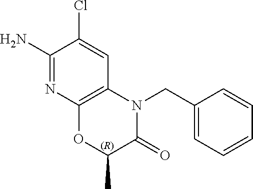

### (3R)-6-amino-1-benzyl-7-chloro-3-methyl-3H-pyrido[2,3-b][1,4]oxazin-2-one

To a stirred mixture of (3R)-1-benzyl-6-bromo-7-chloro-3-methyl-3H-pyrido[2,3-b][1,4]oxazin-2-one (85 mg, 0.231 mmol, 1 equiv) and NaN₃ (45.08 mg, 0.693 mmol, 3 equiv) in EtOH:H₂O=10:1 (5 mL) were added N₁,N₂-dimethylcyclohexane-1,2-diamine (19.73 mg, 0.139 mmol, 0.6 equiv) and CuSO₄ (36.89 mg, 0.231 mmol, 1 equiv) and sodium ascorbate (92.05 mg, 0.462 mmol, 2 equiv) in portions at room temperature under air atmosphere. The resulting mixture was stirred for 1 h at 80° C. under nitrogen atmosphere. After cooling down to room temperature, the reaction quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to afford (3R)-6-amino-1-benzyl-7-chloro-3-methyl-3H-pyrido[2,3-b][1,4]oxazin-2-one (56 mg, 80.0%) as a yellow solid. LC/MS (ESI): mass calcd. for C₁₅H₁₄ClN₃O₂:303.1 m/z, found: 304.25 [M+H]⁺.

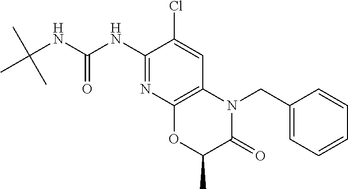 [SMILES: C[C@H]1Oc2nc(NC(=O)NC(C)(C)C)c(Cl)cc2N(Cc2ccccc2)C1=O]

### 1-[(3R)-1-benzyl-7-chloro-3-methyl-2-oxo-3H-pyrido[2,3-b][1,4]oxazin-6-yl]-3-tert-butylurea

To a stirred solution of (3R)-6-amino-1-benzyl-7-chloro-3-methyl-3H-pyrido[2,3-b][1,4]oxazin-2-one (50 mg, 0.165 mmol, 1 equiv) and triphosgene (61.79 mg, 0.209 mmol, 1.2 equiv) in DCM (5 mL) was added TEA (49.97 mg, 0.495 mmol, 3 equiv) dropwise at 0° C. under nitrogen atmosphere. The resulting mixture was stirred for 30 min at room temperature under nitrogen atmosphere. To the above mixture was added 2-methylpropan-2-amine (12.04 mg, 0.165 mmol, 1 equiv) dropwise at room temperature. The resulting mixture was stirred for additional 30 min at room temperature. The reaction was quenched with water (20 mL) and extracted with DCM (3×20 mL). The combined extracts were washed with water, brine, dried over Na₂SO₄, filtered and concentrated. The residue was purified by preparative HPLC using a YMC-Actus Triart Cis ExRS 150 mm×30 mm×5 μm column (eluent: 17% to 42% (v/v) CH₃CN and H₂O with 10 mmol/L NH₄HCO₃) to afford 1-[(3R)-1-benzyl-7-chloro-3-methyl-2-oxo-3H-pyrido[2,3-b][1,4]oxazin-6-yl]-3-tert-butylurea (8.3 mg, 12.37%) as a white solid. LC/MS (ESI): mass calcd. for C₂₀H₂₃ClN₄O₃:402.1 m/z, found: 403.15 [M+H]⁺. ¹H NMR (400 MHz, DMSO-d₆) δ 7.89 (d, J=13.4 Hz, 2H), 7.62 (s, 1H), 7.32-7.40 (m, 2H), 7.19-7.32 (m, 3H), 5.16-5.23 (m, 3H), 1.56 (d, J=6.8 Hz, 3H), 1.32 (s, 9H).

### Example 356: (R)-1-(tert-butyl)-3-(4-(3-(difluoromethyl)benzyl)-8-fluoro-2-methyl-3-oxo-6-(trifluoromethyl)-3,4-dihydro-2H-benzo[b][1,4]oxazin-7-yl)urea

### Synthetic Scheme

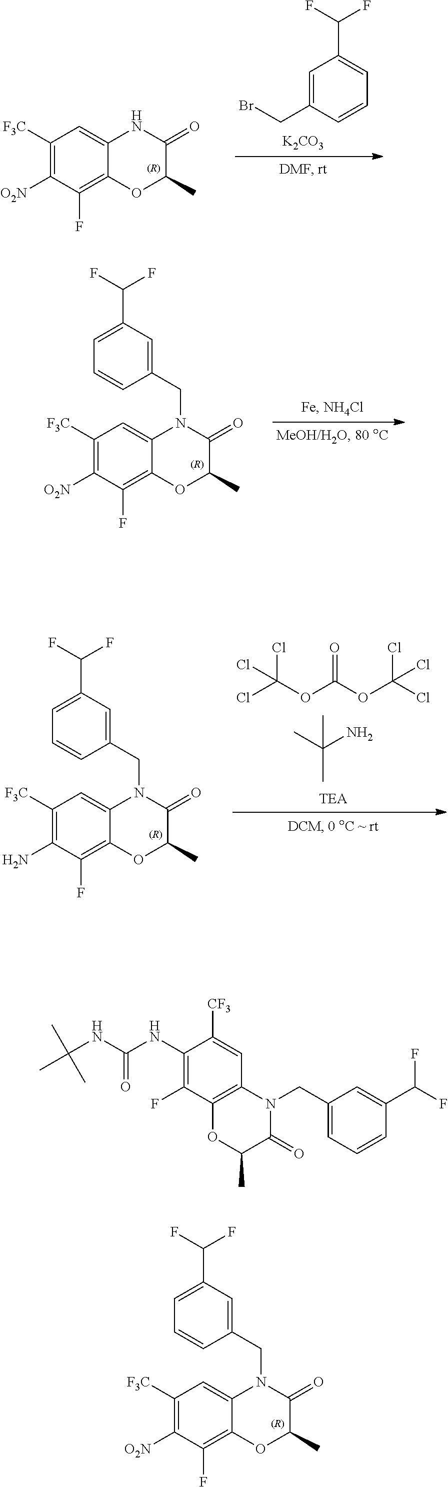

### (R)-4-(3-(difluoromethyl)benzyl)-8-fluoro-2-methyl-7-nitro-6-(trifluoromethyl)-2H-benzo[b][1,4]oxazin-3(4H)-one

To a solution of (2R)-8-fluoro-2-methyl-7-nitro-6-(trifluoromethyl)-2,4-dihydro-1,4-benzoxazin-3-one (100 mg, 0.340 mmol, 1 equiv) in DMF (10 mL) was added 1-(bromomethyl)-3-(difluoromethyl)benzene (112.72 mg, 0.510 mmol, 1.5 equiv) and K₂CO₃ (141.98 mg, 1.020 mmol, 3 equiv). The reaction was stirred at rt for 1 h. The reaction was monitored by TLC. Quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give (2R)-4-{[3-(difluoromethyl)phenyl]methyl}-8-fluoro-2-methyl-7-nitro-6-(trifluoromethyl)-2H-1,4-benzoxazin-3-one (140 mg, 94.83%) as a yellow solid.

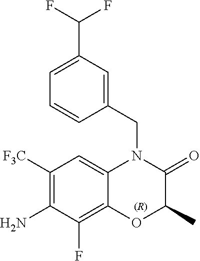

### (R)-7-amino-4-(3-(difluoromethyl)benzyl)-8-fluoro-2-methyl-6-(trifluoromethyl)-2H-benzo[b][1,4]oxazin-3(4H)-one

To a solution of (2R)-4-{[3-(difluoromethyl)phenyl]methyl}-8-fluoro-2-methyl-7-nitro-6-(trifluoromethyl)-2H-1,4-benzoxazin-3-one (130 mg, 0.299 mmol, 1 equiv) in MeOH (5 mL) and H₂O (0.5 mL) was added Fe (167.16 mg, 2.990 mmol, 10 equiv) and NH₄C₁ (160.12 mg, 2.990 mmol, 10 equiv). The reaction was stirred at 80° C. for 1 h. The mixture was allowed to cool down to rt. The resulting mixture was filtered, the filter cake was washed with MeOH (3×30 mL). The filtrate was concentrated under reduced pressure. Quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give (2R)-7-amino-4-{[3-(difluoromethyl)phenyl]methyl}-8-fluoro-2-methyl-6-(trifluoromethyl)-2H-1,4-benzoxazin-3-one (100 mg, 82.63%) as a yellow solid. LC/MS: mass calcd. for C₁₈H₁₄F₆N₂O₂:404.10 m/z, found: 405.05 [M+H]⁺.

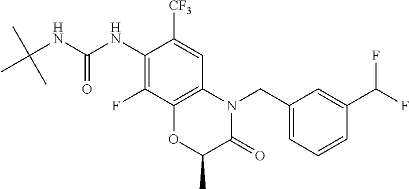 [SMILES: C[C@H]1Oc2c(cc(C(F)(F)F)c(NC(=O)NC(C)(C)C)c2F)N(Cc2cccc(C(F)F)c2)C1=O]

### (R)-1-(tert-butyl)-3-(4-(3-(difluoromethyl)benzyl)-8-fluoro-2-methyl-3-oxo-6-(trifluoromethyl)-3,4-dihydro-2H-benzo[b][1,4]oxazin-7-yl)urea

To a solution of (2R)-7-amino-4-{[3-(difluoromethyl)phenyl]methyl}-8-fluoro-2-methyl-6-(trifluoromethyl)-2H-1,4-benzoxazin-3-one (150 mg, 0.371 mmol, 1 equiv) in DCM (5 mL) was added triphosgene (110.09 mg, 0.371 mmol, 1 equiv) under N₂ at 0° C. The reaction was stirred at 0° C. under N₂ for 1 h. To the above mixture was added TEA (112.63 mg, 1.113 mmol, 3 equiv) at 0° C. The resulting mixture was stirred for additional 1 h at rt. Then, to the above mixture was added erbumine (81.40 mg, 1.113 mmol, 3 equiv). The resulting mixture was stirred for additional 1 h at rt. Quenched with water (10 mL) and extracted with DCM (3×10 mL). The combined extracts were washed with water, brine, dried over Na₂SO₄, filtered and concentrated. The residue was purified by preparative HPLC using a XBridge Prep C18 OBD Column, 19*250 mm, 5 μm column (eluent: 49% to 79% (v/v) CH₃CN and Water (10 mmol/L NH₄HCO_{3+0.1}% NH₃·H₂O)) to afford the title compound 3-tert-butyl-1-[(2R)-4-{[3-(difluoromethyl)phenyl]methyl}-8-fluoro-2-methyl-3-oxo-6-(trifluoromethyl)-2H-1,4-benzoxazin-7-yl]urea (20.3 mg, 10.79%) as a white solid. LC/MS: mass calcd. for C₂₃H₂₃F₆N₃O₃:503.16 m/z, found: 504.20 [M+H]⁺. ¹H NMR (400 MHz, DMSO-d₆) δ 7.46 (s, 1H), 7.31-7.40 (m, 2H), 7.23-7.31 (m, 3H), 7.06 (d, J=1.6 Hz, 1H), 6.27 (s, 1H), 5.28 (d, J=16.4 Hz, 1H), 5.21 (d, J=16.4 Hz, 1H), 5.14 (q, J=6.7 Hz, 1H), 1.59 (d, J=6.7 Hz, 3H), 1.25 (s, 9H). ¹⁹F NMR (376 MHz, DMSO) δ(ppm): −59.0147, −136.7360.

### Example 357: (S)-1-(tert-butyl)-3-(1-(1-(3-chlorophenyl)ethyl)-7-fluoro-2-oxo-1,2-dihydroquinoxalin-6-yl) Urea

### Synthetic Scheme

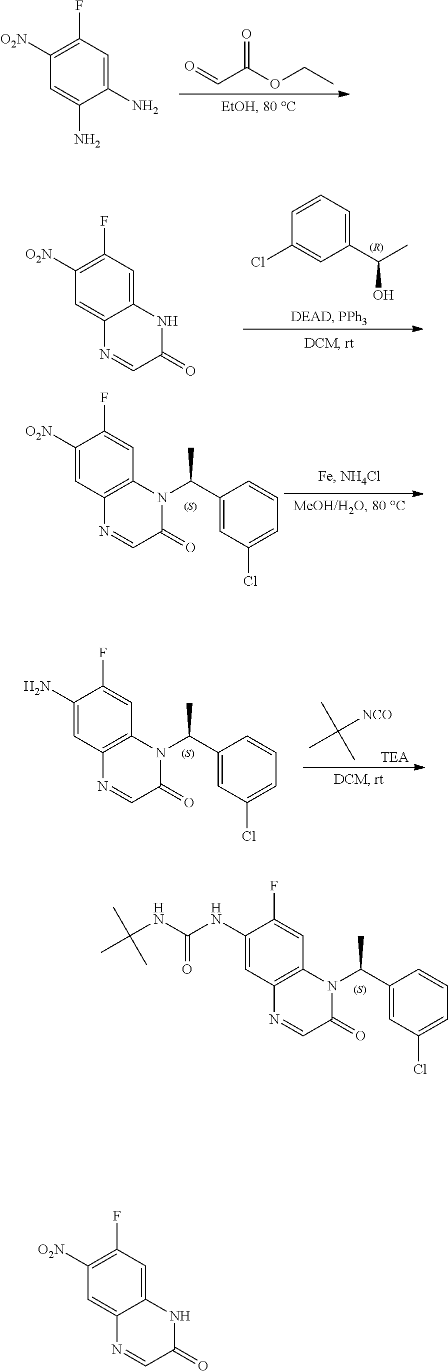

### 7-fluoro-6-nitroquinoxalin-2(1H)-one

To a solution of 4-fluoro-5-nitrobenzene-1,2-diamine (2 g, 11.687 mmol, 1 equiv) in ethyl alcohol (10 mL) was added ethyl glyoxylate (1.19 g, 11.687 mmol, 1 equiv). The resulting mixture was stirred for 2 h at 80° C. The reaction mixture was quenched by water and extracted with DCM (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 7-fluoro-6-nitroquinoxalin-2(1H)-one (1.7 g, 69.55%) as a white solid. MS (ESI): mass calcd. for C₈H₄FN₃O₃:209.17 m/z, found: 208 [M−H]⁻.

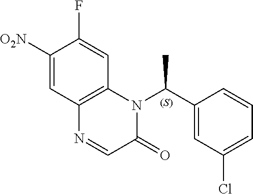

### (S)-1-(1-(3-chlorophenyl)ethyl)-7-fluoro-6-nitroquinoxalin-2(1H)-one

To a solution of 7-fluoro-6-nitroquinoxalin-2(1H)-one (1 g, 4.782 mmol, 1 equiv) in DCM (40 mL) was added (1R)-1-(3-chlorophenyl)ethanol (0.75 g, 4.782 mmol, 1 equiv) PPh₃ (1.88 g, 7.173 mmol, 1.5 equiv) and DEAD (1.25 g, 7.173 mmol, 1.5 equiv). The resulting mixture was stirred for 2 h at 25° C. under N₂ and stirred until the starting material was totally consumed by TLC. The reaction mixture was quenched by water and extracted with DCM (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford (S)-1-(1-(3-chlorophenyl)ethyl)-7-fluoro-6-nitroquinoxalin-2(1H)-one (400 mg, 24.06%) as a white solid. MS (ESI): mass calcd. for C₁₆H₁₁ClFN₃O₃: 347.17 m/z, found: 348.20 [M+H]⁺.

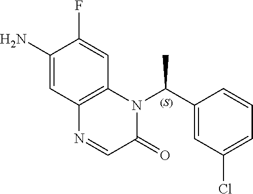

### (S)-6-amino-1-(1-(3-chlorophenyl)ethyl)-7-fluoroquinoxalin-2(1H)-one

To a solution of (S)-1-(1-(3-chlorophenyl)ethyl)-7-fluoro-6-nitroquinoxalin-2(1H)-one (300 mg, 0.863 mmol, 1 equiv) in EtOH (1 mL) and H₂O (0.1 mL) was added Fe (481 mg, 8.630 mmol, 10 equiv) and NH₄Cl (461 mg, 8.630 mmol, 10 equiv). The resulting mixture was stirred for 2 h at 80° C. and stirred until the starting material was totally consumed by TLC. The reaction mixture was quenched by water and extracted with DCM (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford (S)-6-amino-1-(1-(3-chlorophenyl)ethyl)-7-fluoroquinoxalin-2(1H)-one (120 mg, 43.77%) as a white solid. MS (ESI): mass calcd. for C₁₆H₁₃ClFN₃O: 317.17 m/z, found: 318.20 [M+H]⁺.

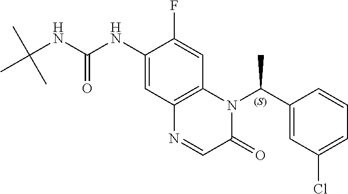

### (S)-1-(tert-butyl)-3-(1-(1-(3-chlorophenyl)ethyl)-7-fluoro-2-oxo-1,2-dihydroquinoxalin-6-yl)urea

To a mixture of (S)-6-amino-1-(1-(3-chlorophenyl)ethyl)-7-fluoroquinoxalin-2(1H)-one (60 mg, 0.189 mmol, 1 equiv) and TEA (57 mg, 0.567 mmol, 3 equiv) in DCM (4 mL) was added 2-isocyanato-2-methylpropane (56 mg, 0.567 mmol, 3 equiv) at 0° C. The reaction was stirred for overnight at rt. The reaction mixture was quenched by water (10 mL) and extracted with DCM (3×10 mL). The organic layers were combined, dried over anhydrous sodium sulfate, filtered and concentrated. The crude product was purified by silica gel column with PE/EA (1:1) & Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 53% B in 9 min, 53% B; Wave Length: 254/220 nm; RT1(min): 8.52; Number Of Runs: 0) to afford (S)-1-(tert-butyl)-3-(1-(1-(3-chlorophenyl)ethyl)-7-fluoro-2-oxo-1,2-dihydroquinoxalin-6-yl)urea (12.3 mg, 15.09%) as a white solid. MS (ESI): mass calcd. For C₂₁H₂₂ClFN₄O₂: 416.17 m/z, found: 417.20 [M+H]⁺. ¹H NMR (400 MHz, DMSO-d₆) δ 8.57 (d, J=8.7 Hz, 1H), 8.19 (s, 1H), 8.25 (s, 1H), 7.32-7.42 (m, 4H), 7.20-7.29 (m, 1H), 6.58 (s, 1H), 6.44 (s, 1H), 1.87 (d, J=7.0 Hz, 3H), 1.30 (s, 9H).

### Example 358: 3-tert-butyl-1-{1-[(1S)-1-(3-chlorophenyl)ethyl]-2-oxo-7-(trifluoromethyl)quinoxalin-6-yl}urea

### Synthetic Scheme

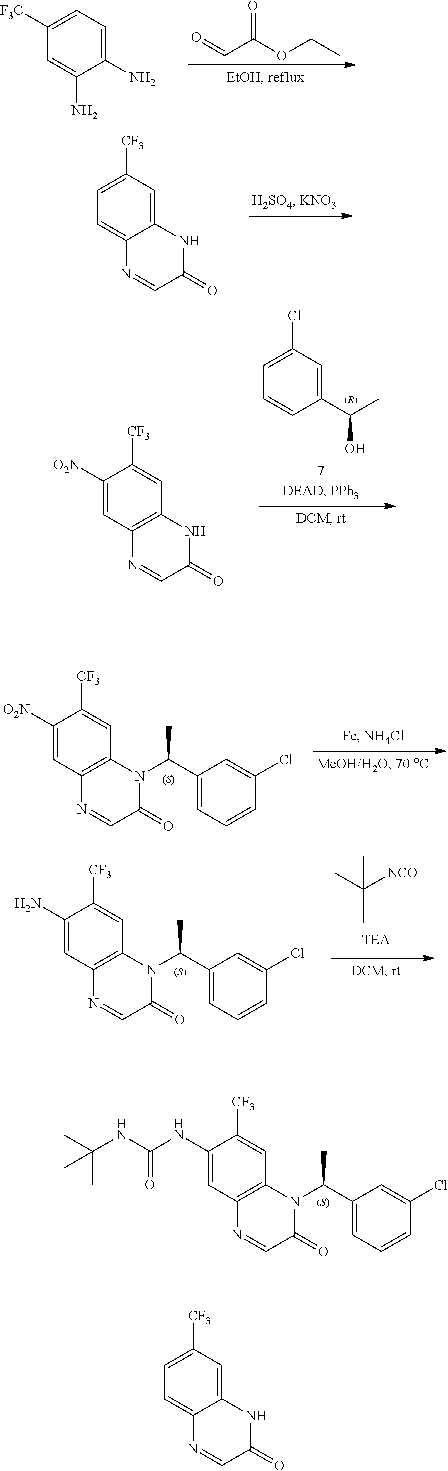

### 7-(trifluoromethyl)-1H-quinoxalin-2-one

To a stirred solution of 4-(trifluoromethyl)benzene-1,2-diamine (2 g, 11.354 mmol, 1 equiv) in EtOH (20 mL) was added ethyl glyoxylate (1.16 g, 11.354 mmol, 1 equiv) in portions at room temperature under air atmosphere. The resulting mixture was stirred for 3 h at 60° C. under air atmosphere. The reaction was monitored by LCMS. The mixture was allowed to cool down to room temperature. Quenched with water (100 mL) and extracted with EA (3×100 mL). The combined extracts were washed with water, brine, dried over Na₂SO₄, filtered and concentrated. The residue was purified by silica gel column chromatography, eluted with PE/EA (0-100%) to afford 7-(trifluoromethyl)-1H-quinoxalin-2-one (1.2 g, 49.35%) as a yellow solid. LC/MS (ESI): mass calcd. for C₉H₅F₃N₂O:214.04 m/z, found: 215.0 [M+H]⁺.

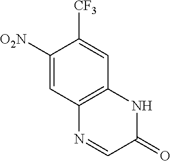 [SMILES: O=c1cnc2cc([N+](=O)[O-])c(C(F)(F)F)cc2[nH]1]

### 6-nitro-7-(trifluoromethyl)-1H-quinoxalin-2-one

To a stirred solution of 7-(trifluoromethyl)-1H-quinoxalin-2-one (1.1 g, 5.137 mmol, 1 equiv) in H₂SO₄ (11 mL) was added KNO₃ (1.56 g, 15.411 mmol, 3 equiv) in portions at 0° C. under air atmosphere. The resulting mixture was stirred for 2 h at room temperature under air atmosphere. The reaction was monitored by LCMS. The reaction was quenched with water/Ice (100 mL) at 0° C., and extracted with EA (3×100 mL). The combined extracts were washed with water, brine, dried over Na₂SO₄, filtered and concentrated. The residue was purified by silica gel column chromatography, eluted with PE/EA (0-100%) to afford 6-nitro-7-(trifluoromethyl)-1H-quinoxalin-2-one (600 mg, 45.07%) as a brown solid. LC/MS (ESI): mass calcd. for C₉H₄F₃N₃O₃:259.02 m/z, found: 260.0 [M+H]⁺.

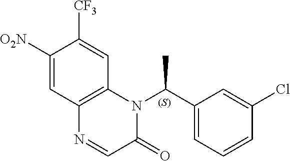

### 1-[(1S)-1-(3-chlorophenyl)ethyl]-6-nitro-7-(trifluoromethyl)quinoxalin-2-one

To a stirred solution of 6-nitro-7-(trifluoromethyl)-1H-quinoxalin-2-one (600 mg, 2.315 mmol, 1 equiv) and (1R)-1-(3-chlorophenyl)ethanol (362.60 mg, 2.315 mmol, 1 equiv) in DCM (10 mL) was added PPh₃ (910.93 mg, 3.473 mmol, 1.5 equiv) in portions at 0° C. under nitrogen atmosphere. The resulting mixture was stirred for 15 min at room temperature under nitrogen atmosphere. To the above mixture was added DEAD (403.23 mg, 2.315 mmol, 1 equiv) in portions at 0° C. The resulting mixture was stirred for additional 2 h at room temperature. The reaction was monitored by LCMS. Quenched with water (50 mL) and extracted with DCM (3×50 mL). The combined extracts were washed with water, brine, dried over Na₂SO₄, filtered and concentrated. The residue was purified by silica gel column chromatography, eluted with PE/EA (0-100%) to afford 1-[(1S)-1-(3-chlorophenyl)ethyl]-6-nitro-7-(trifluoromethyl)quinoxalin-2-one (110 mg, 11.94%) as a yellow solid. LC/MS (ESI): mass calcd. for C₁₇H₁₁ClF₃N₃O₃:397.04 m/z, found: 398.0 [M+H]⁺.

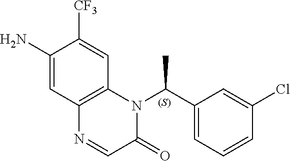

### 6-amino-1-[(1S)-1-(3-chlorophenyl)ethyl]-7-(trifluoromethyl)quinoxalin-2-one

To a stirred solution of 1-[(1S)-1-(3-chlorophenyl)ethyl]-6-nitro-7-(trifluoromethyl)quinoxalin-2-one (100 mg, 0.251 mmol, 1 equiv) in MeOH/H₂O (10:1, 5 mL) was added Fe (14.04 mg, 0.250 mmol, 10 equiv) and NH₄C₁ (134.48 mg, 2.510 mmol, 10 equiv) in portions at room temperature under air atmosphere. The resulting mixture was stirred for 2 h at 70° C. under air atmosphere. The reaction was monitored by LCMS. The mixture was allowed to cool down to room temperature. Quenched with water (10 mL) and extracted with EA (3×10 mL). The combined extracts were washed with water, brine, dried over Na₂SO₄, filtered and concentrated. The residue was purified by silica gel column chromatography, eluted with PE/EA (0-100%) to afford 6-amino-1-[(1S)-1-(3-chlorophenyl)ethyl]-7-(trifluoromethyl)quinoxalin-2-one (55 mg, 59.48%) as a yellow solid. LC/MS (ESI): mass calcd. for C₁₇H₁₄F₃N₃O:367.1 m/z, found: 368.05 [M+H]⁺.

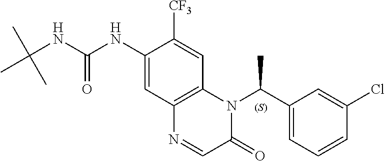

### 3-tert-butyl-1-{1-[(1S)-1-(3-chlorophenyl)ethyl]-2-oxo-7-(trifluoromethyl)quinoxalin-6-yl}urea

To a stirred solution of 6-amino-1-[(1S)-1-(3-chlorophenyl)ethyl]-7-(trifluoromethyl)quinoxalin-2-one (50 mg, 0.136 mmol, 1 equiv) in DCM (4 mL) was added TEA (41.27 mg, 0.408 mmol, 3 equiv) and 2-isocyanato-2-methylpropane (40.43 mg, 0.408 mmol, 3 equiv) in portions at room temperature under air atmosphere. The resulting mixture was stirred for 1 days at room temperature under air atmosphere. The reaction was monitored by LCMS. Quenched with water (5 mL) and extracted with DCM (3×5 mL). The combined extracts were washed with water, brine, dried over Na₂SO₄, filtered and concentrated. The crude product was purified by Prep-HPLC using a X Bridge Prep OBD C18 150 mm×30 mm×5 μm column (eluent: 43% to 59% (v/v) CH₃CN and H₂O with 10 mmol/L NH₄HCO_{3+0.05}% NH₃H₂O) to afford 3-tert-butyl-1-{1-[(1S)-1-(3-chlorophenyl)ethyl]-2-oxo-7-(trifluoromethyl)quinoxalin-6-yl}urea (2.7 mg, 4.23%) as a yellow solid. LC/MS (ESI): mass calcd. for C₂₂H₂₂C₁F₃N₄O₂:466.1 m/z, found: 467.15 [M+H]⁺. ¹H NMR (300 MHz, DMSO-d₆) δ 8.37 (d, J=3.0 Hz, 2H), 7.68 (s, 1H), 7.44-7.37 (m, 3H), 7.29 (s, 2H), 6.92 (s, 1H), 6.55 (d, J=7.2 Hz, 1H), 1.85 (d, J=7.2 Hz, 3H), 1.29 (s, 9H). ¹⁹F NMR (282 MHz, DMSO-d₆) δ −60.17.

### Example 359: cyclopentyl (S)-(2-oxo-1-(1-(3-(trifluoromethoxy)phenyl)ethyl)-1,2-dihydroquinoxalin-6-yl)carbamate

### Synthetic Scheme

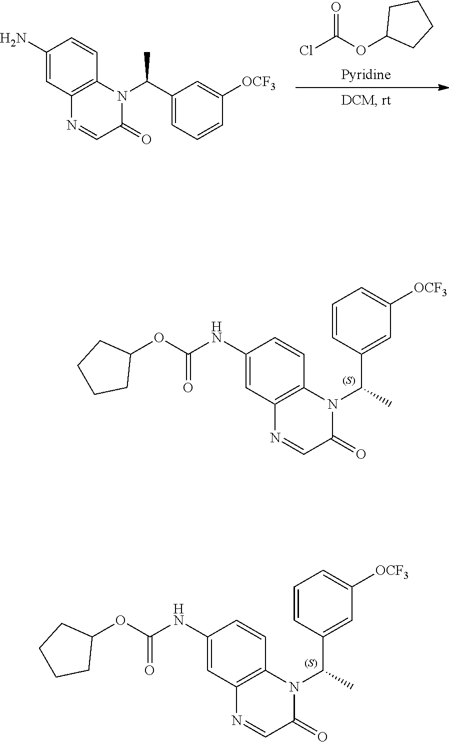

### cyclopentyl (S)-(2-oxo-1-(1-(3-(trifluoromethoxy)phenyl)ethyl)-1,2-dihydroquinoxalin-6-yl)carbamate

To a solution of 6-amino-1-[(1S)-1-[3-(trifluoromethoxy)phenyl]ethyl]quinoxalin-2-one (80 mg, 0.229 mmol, 1 equiv) in DCM (5 mL) was added cyclopentyl carbonochloridate (51.05 mg, 0.344 mmol, 1.5 equiv) and pyridine (54.35 mg, 0.687 mmol, 3 equiv) at 0° C. The reaction was stirred at rt for 1 h. Quenched with water (10 mL) and extracted with DCM (3×10 mL). The combined extracts were washed with water, brine, dried over Na₂SO₄, filtered and concentrated. The residue was purified by preparative HPLC using a XBridge Prep OBD C18 Column, 30*150 mm, 5 μm column (eluent: 49% to 63% (v/v) CH₃CN and Water (10 mmol/L NH₄HCO₃+0.05% NH₃·H₂O)) to afford the title compound cyclopentyl N-{2-oxo-1-[(1S)-1-[3-(trifluoromethoxy)phenyl]ethyl]quinoxalin-6-yl}carbamate (32.2 mg, 30.22%) as a yellow solid. LC/MS: mass calcd. for C₂₃H₂₂F₃N₃O₄:461.16 m/z, found: 462.10 [M+H]⁺. ¹H NMR (400 MHz, DMSO-d₆) δ 9.75 (s, 1H), 8.26 (s, 1H), 8.00 (s, 1H), 7.43-7.51 (m, 1H), 7.46 (s, 1H), 7.29 (q, J=5.7 Hz, 3H), 7.17 (s, 1H), 6.57 (s, 1H), 5.00-5.15 (m, 1H), 1.85-1.91 (m, 5H), 1.65-1.72 (m, 4H), 1.60 (d, J=9.4 Hz, 2H). ¹⁹F NMR (376 MHz, DMSO) δ(ppm): −56.6795.

### Example 360: (S)-1-(tert-butyl)-3-(7-(hydroxymethyl)-2-oxo-1-(1-phenylethyl)-1,2-dihydroquinoxalin-6-yl)urea

### Synthetic Scheme

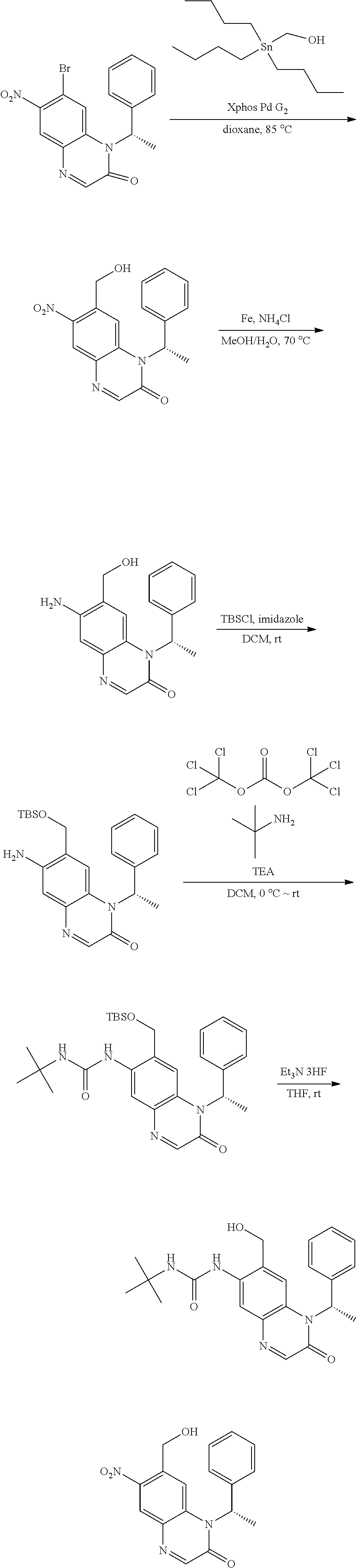

### (S)-7-(hydroxymethyl)-6-nitro-1-(1-phenylethyl)quinoxalin-2(1H)-one

To a solution of 7-bromo-6-nitro-1-[(1S)-1-phenylethyl]quinoxalin-2-one (300 mg, 0.802 mmol, 1 equiv) in dioxane (3 mL) was added (tributylstannyl)methanol (386.14 mg, 1.203 mmol, 1.5 equiv) and XPhos Pd G2 (63.08 mg, 0.080 mmol, 0.1 equiv). The reaction was stirred at 90° C. for 12 h under N₂. The mixture was allowed to cool down to rt. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give 7-(hydroxymethyl)-6-nitro-1-[(1S)-1-phenylethyl]quinoxalin-2-one (40 mg, 15.34%) as a white solid. MS (ESI): mass calcd. for C₁₇H₁₅N₃O₄: 325.11 m/z, found 323.95 [M−H]⁻.

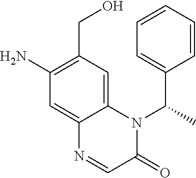 [SMILES: C[C@@H](c1ccccc1)n1c(=O)cnc2cc(N)c(CO)cc21]

### (S)-6-amino-7-(hydroxymethyl)-1-(1-phenylethyl)quinoxalin-2(1H)-one

To a solution of 7-(hydroxymethyl)-6-nitro-1-[(1S)-1-phenylethyl]quinoxalin-2-one (40 mg, 0.123 mmol, 1 equiv) in MeOH (2 mL)/H₂O (0.2 mL) was added Fe (68.66 mg, 1.230 mmol, 10 equiv) and NH₄C₁ (65.77 mg, 1.230 mmol, 10 equiv). The reaction was stirred at 70° C. for 3 h. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to 6-amino-7-(hydroxymethyl)-1-[(1S)-1-phenylethyl]quinoxalin-2-one (25 mg, 68.84%) as a white solid. MS (ESI): mass calcd. for C₁₇H₁₇N₃O₂: 295.13 m/z, found 296.10 [M+H]⁺.

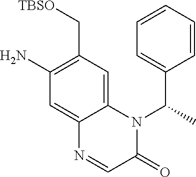 [SMILES: C[C@@H](c1ccccc1)n1c(=O)cnc2cc(N)c(CO[Si](C)(C)C(C)(C)C)cc21]

### (S)-6-amino-7-(((tert-butyldimethylsilyl)oxy)methyl)-1-(1-phenylethyl)quinoxalin-2(1H)-one

To a solution of 6-amino-7-(hydroxymethyl)-1-[(1S)-1-phenylethyl]quinoxalin-2-one (70 mg, 0.237 mmol, 1 equiv) in DCM (3 mL) was added TBSCl (53.58 mg, 0.355 mmol, 1.5 equiv) and imidazole (48.41 mg, 0.711 mmol, 3 equiv). The reaction was stirred at rt for 3 h. Quenched with water (10 mL) and extracted with DCM (3×10 mL). The combined extracts were washed with water, brine, dried over Na₂SO₄, filtered and concentrated. The residue was purified by column chromatography on silica gel with EA/PE (0-100%) to give 6-amino-7-{[(tert-butyldimethylsilyl)oxy]methyl}-1-[(1S)-1-phenylethyl]quinoxalin-2-one (75 mg, 77.25%) as a white solid. MS (ESI): mass calcd. for C₂₃H₃₁N₃O₂Si: 409.22 m/z, found 410.25 [M+H]⁺.

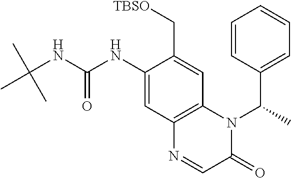 [SMILES: C[C@@H](c1ccccc1)n1c(=O)cnc2cc(NC(=O)NC(C)(C)C)c(CO[Si](C)(C)C(C)(C)C)cc21]

### (S)-1-(tert-butyl)-3-(7-(((tert-butyldimethylsilyl)oxy)methyl)-2-oxo-1-(1-phenylethyl)-1,2-dihydroquinoxalin-6-yl)urea

To a solution of 6-amino-7-{[(tert-butyldimethylsilyl)oxy]methyl}-1-[(1S)-1-phenylethyl]quinoxalin-2-one (50 mg, 0.122 mmol, 1 equiv) in DCM (1 mL) was added triphosgene (36.22 mg, 0.122 mmol, 1 equiv). The reaction was stirred at 0° C. under N₂ for 1 h. The reaction was added TEA (74.12 mg, 0.732 mmol, 6 equiv) and stirred at rt under N₂ for 1 h. The reaction was added 2-methylpropan-2-amine (26.78 mg, 0.366 mmol, 3 equiv) and stirred at rt under N₂ for 1 h. Quenched with water (5 mL) and extracted with DCM (3×5 mL). The combined extracts were washed with water, brine, dried over Na₂SO₄, filtered and concentrated. The residue was purified by column chromatography on silica gel with EA/PE (0-100%) to give 3-tert-butyl-1-(7-{[(tert-butyldimethylsilyl)oxy]methyl}-2-oxo-1-[(1S)-1-phenylethyl]quinoxalin-6-yl)urea (20 mg, 32.21%) as a white solid. LC/MS (ESI): mass calcd. for C₂₈H₄₀N₄O₃Si: 508.29 m/z, found: 509.35 [M+H]⁺.

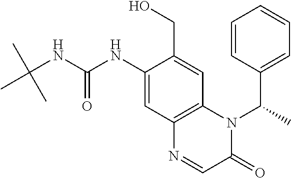 [SMILES: C[C@@H](c1ccccc1)n1c(=O)cnc2cc(NC(=O)NC(C)(C)C)c(CO)cc21]

### 1-(1-benzyl-5-chloro-2-oxo-1,2,3,4-tetrahydroquinolin-6-yl)-3-(tert-butyl)urea

To a solution of 3-tert-butyl-1-(7-{[(tert-butyldimethylsilyl)oxy]methyl}-2-oxo-1-[(1S)-1-phenylethyl]quinoxalin-6-yl)urea (20 mg, 0.039 mmol, 1 equiv) in THF (1 mL) was added Triethylamine trihydrofluoride (9.51 mg, 0.058 mmol, 1.5 equiv). The reaction was stirred at rt for 1 h. Quenched with water (5 mL) and extracted with EA (3×5 mL). The combined extracts were washed with water, brine, dried over Na₂SO₄, filtered and concentrated. The residue was purified by preparative HPLC using a XBridge Prep OBD C18 Column, 30*150 mm, 5 μm (eluent: 22% to 38% (v/v) CH₃CN and H₂O with 10 mmol/L NH₄HCO₃) to afford the title compound 3-tert-butyl-1-[7-(hydroxymethyl)-2-oxo-1-[(1S)-1-phenylethyl]quinoxalin-6-yl]urea (6.2 mg, 39.70%) as a white solid. LC/MS (ESI): mass calcd. for C₂₂H₂₆N₄O₃: 394.20 m/z, found: 395.15 [M+H]⁺. ¹H NMR (300 MHz, DMSO-d₆) δ 8.21-8.27 (d, J=18 Hz, 2H), 7.58 (s, 1H), 7.21-7.38 (m, 6H), 6.60 (s, 2H), 5.29-5.33 (t, J=12 Hz, 1H), 4.26-4.42 (m, 2H), 1.87-1.89 (d, J=6 Hz, 3H), 1.27 (s, 9H).

### Example 361: 3-tert-butyl-1-{1-[(1S)-1-(3-chlorophenyl)ethyl]-7-cyano-2-oxoquinoxalin-6-yl}urea

### Synthetic Scheme

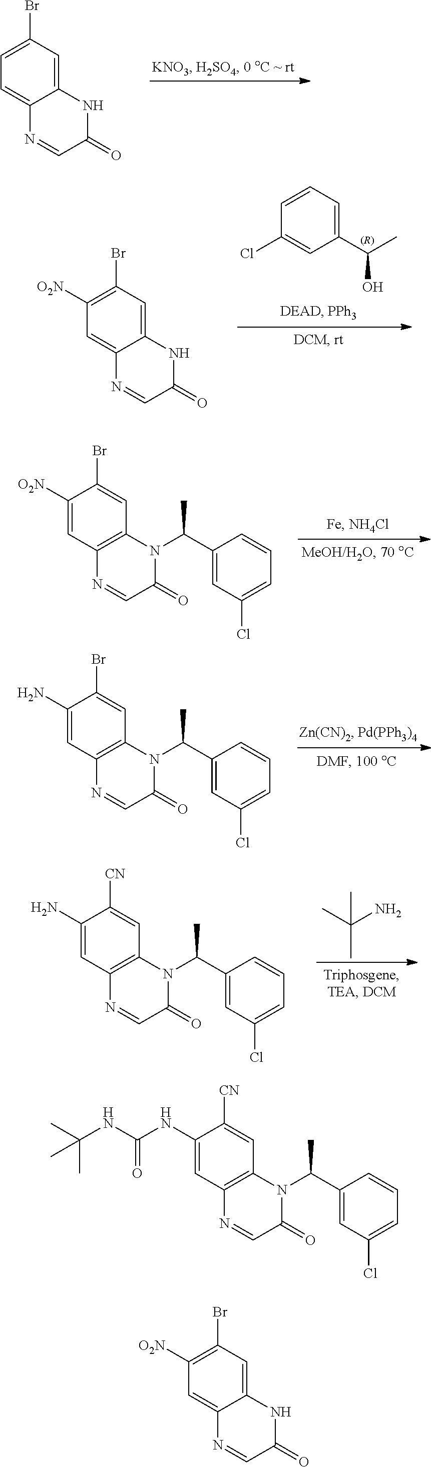

### 7-bromo-6-nitro-1H-quinoxalin-2-one

To a solution of 7-bromo-1H-quinoxalin-2-one (5 g, 22.218 mmol, 1 equiv) in H₂SO₄ (50 mL) was added KNO₃ (2.92 g, 28.883 mmol, 1.3 equiv) at 0° C. The reaction was stirred at 0° C. for 1 h.

Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give 7-bromo-6-nitro-1H-quinoxalin-2-one (4.5 g, 75.00%) as a yellow solid. MS (ESI): mass calcd. for C₈H₄BrN₃O₃:268.94 m/z, found: 269.94, 271.96 [M+H, M+H+2]⁺.

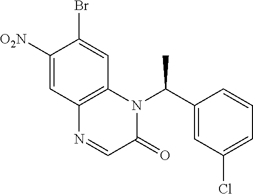 [SMILES: C[C@@H](c1cccc(Cl)c1)n1c(=O)cnc2cc([N+](=O)[O-])c(Br)cc21]

### 7-bromo-1-[(1S)-1-(3-chlorophenyl)ethyl]-6-nitroquinoxalin-2-one

To a solution of 7-bromo-6-nitro-1H-quinoxalin-2-one (1.5 g, 5.555 mmol, 1 equiv) in DCM (20 mL) was added (1R)-1-(3-chlorophenyl)ethanol (0.87 g, 5.555 mmol, 1 equiv) and PPh₃ (2.19 g, 8.332 mmol, 1.5 equiv). After the mixture was stirred at rt for 1 h, to the above mixture was added DEAD (0.97 g, 5.555 mmol, 1 equiv) dropwise at 0° C. The resulting mixture was stirred for 2 h at 25° C. Quenched with water (100 mL) and extracted with DCM (3×100 mL). The combined extracts were washed with water, brine, dried over Na₂SO₄, filtered and concentrated. The residue was purified by column chromatography on silica gel with EA/PE (0-100%) to give 7-bromo-1-[(1S)-1-(3-chlorophenyl)ethyl]-6-nitroquinoxalin-2-one (210 mg, 9.25%) as a yellow oil. MS (ESI): mass calcd. for C₁₆H₁₁BrClN₃O₃:406.97 m/z, found: 407.97, 409.97 [M+H, M+H+2]⁺.

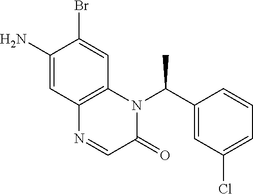 [SMILES: C[C@@H](c1cccc(Cl)c1)n1c(=O)cnc2cc(N)c(Br)cc21]

### 6-amino-7-bromo-1-[(1S)-1-(3-chlorophenyl)ethyl]quinoxalin-2-one

To a solution of 7-bromo-1-[(1S)-1-(3-chlorophenyl)ethyl]-6-nitroquinoxalin-2-one (280 mg, 0.685 mmol, 1 equiv) in MeOH (10 mL) and H₂O (1 mL) was added Fe (382.65 mg, 6.850 mmol, 10 equiv) and NH₄C₁ (366.51 mg, 6.850 mmol, 10 equiv). The reaction was stirred at 70° C. for 2 h. The mixture was allowed to cool down to room temperature and quenched with water (50 mL). The resulting mixture was filtered; the filter cake was washed with EA (50 mL). The filtrate was extracted with EA (3×50 mL). The combined extracts were washed with water, brine, dried over Na₂SO₄, filtered and concentrated. The residue was purified by column chromatography on silica gel with EA/PE (0-100%) to give 6-amino-7-bromo-1-[(1S)-1-(3-chlorophenyl)ethyl]quinoxalin-2-one) as a yellow oil. MS (ESI): mass calcd. for C₁₆H₁₃BrClN₃O:376.99 m/z, found: 377.99, 379.99 [M+H, M+H+2]⁺.

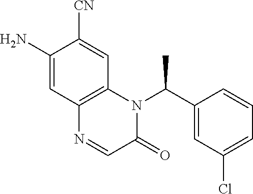 [SMILES: C[C@@H](c1cccc(Cl)c1)n1c(=O)cnc2cc(N)c(C#N)cc21]

### 7-amino-4-[(1S)-1-(3-chlorophenyl)ethyl]-3-oxoquinoxaline-6-carbonitrile

To a solution of 6-amino-7-bromo-1-[(1S)-1-(3-chlorophenyl)ethyl]quinoxalin-2-one (140 mg, 0.370 mmol, 1 equiv) in dimethylformamide (8 mL) was added Zn(CN)₂ (52.10 mg, 0.444 mmol, 1.2 equiv) and Pd(PPh₃)₄ (42.73 mg, 0.037 mmol, 0.1 equiv). The reaction was stirred at 100° C. for 2 h under N₂ atmosphere. The mixture was allowed to cool down to room temperature. Quenched with water (30 mL) and extracted with EA (3×30 mL). The combined extracts were washed with water, brine, dried over Na₂SO₄, filtered and concentrated. The residue was purified by column chromatography on silica gel with EA/PE (0-100%) to give 7-amino-4-[(1S)-1-(3-chlorophenyl)ethyl]-3-oxoquinoxaline-6-carbonitrile (60 mg, 49.97%) as a red solid. MS (ESI): mass calcd. for C₁₇H₁₃ClN₄O:324.08 m/z, found: 325.08 [M+H]⁺.

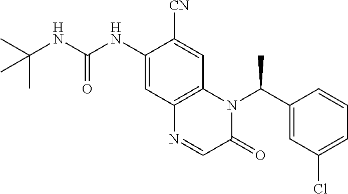 [SMILES: C[C@@H](c1cccc(Cl)c1)n1c(=O)cnc2cc(NC(=O)NC(C)(C)C)c(C#N)cc21]

### 3-tert-butyl-1-{1-[(1S)-1-(3-chlorophenyl)ethyl]-7-cyano-2-oxoquinoxalin-6-yl}urea

To a solution of 7-amino-4-[(1S)-1-(3-chlorophenyl)ethyl]-3-oxoquinoxaline-6-carbonitrile (50 mg, 0.154 mmol, 1 equiv) in DCM (8 mL) was added triphosgene (22.84 mg, 0.077 mmol, 0.5 equiv) at 0° C. under N₂ atmosphere. The resulting mixture was stirred for 2 h at 25° C. Then TEA (46.74 mg, 0.462 mmol, 3 equiv) was added into above system at 0° C. The resulting mixture was stirred for 1 h at 25° C. To the above mixture was added erbumine (16.89 mg, 0.231 mmol, 1.5 equiv) dropwise at 0° C. The reaction was stirred at 25° C. for 2 h. Quenched with water (30 mL) and extracted with DCM (3×20 mL). The combined extracts were washed with water, brine, dried over Na₂SO₄, filtered and concentrated. The residue was purified by preparative HPLC using aXselect CSH C18 OBD 30*150 mm, 5 μm column (eluent: 42% to 82% (v/v) CH₃CN and H₂O with 10 mmol/L NH₄HCO₃) to afford the title compound 3-tert-butyl-1-{1-[(1S)-1-(3-chlorophenyl)ethyl]-7-cyano-2-oxoquinoxalin-6-yl}urea (14.7 mg, 21.84%) as a yellow solid. LC/MS (ESI): mass calcd. for C₂₂H₂₂ClN₅O₂:423.15 m/z, found: 424.15 [M+H]⁺. ¹H NMR (400 MHz, DMSO-d₆) δ 8.49 (s, 1H), 8.40 (s, 1H), 8.32 (s, 1H), 7.53-7.90 (m, 1H), 7.40-7.45 (m, 1H), 7.32-7.40 (m, 2H), 7.23-7.40 (m, 1H), 6.96 (s, 1H), 6.38 (s, 1H), 1.87 (d, J=7.0 Hz, 3H), 1.31 (s, 9H).

### Example 362: 1-[(3R)-1-benzyl-7-cyano-3-methyl-2-oxo-3H-pyrido[2,3-b][1,4]oxazin-6-yl]-3-tert-butylurea

### Synthetic Scheme

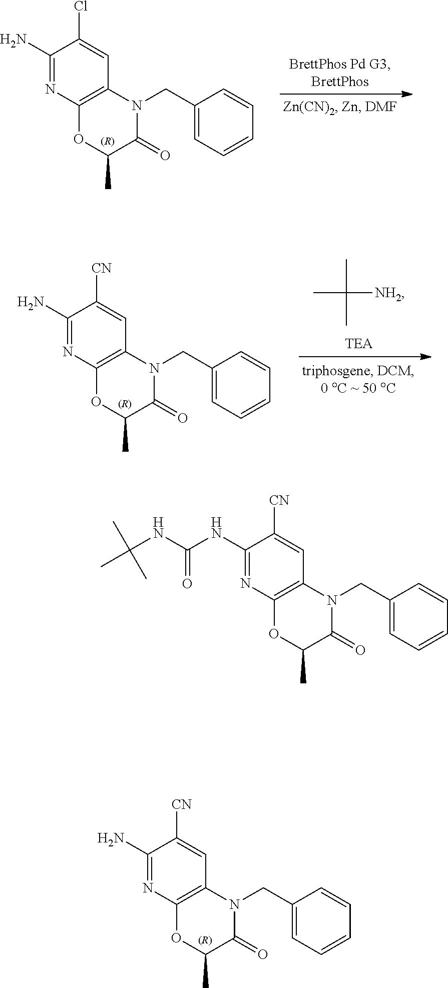

### (3R)-6-amino-1-benzyl-3-methyl-2-oxo-3H-pyrido[2,3-b][1,4]oxazine-7-carbonitrile

To a stirred mixture of (3R)-6-amino-1-benzyl-7-chloro-3-methyl-3H-pyrido[2,3-b][1,4]oxazin-2-one (50 mg, 0.165 mmol, 1 equiv) and Zn(CN)₂ (38.66 mg, 0.330 mmol, 2 equiv) in DMF (5 mL) was added BrettPhos Pd G3 (298.44 mg, 0.330 mmol, 02 equiv) and BrettPhos (17.67 mg, 0.033 mmol, 0.2 equiv) and Zn (3.23 mg, 0.050 mmol, 0.3 equiv) in portions at room temperature under air atmosphere. The resulting mixture was stirred for 1 h at 120° C. under nitrogen atmosphere. After cooling down to room temperature, the reaction was quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to afford (3R)-6-amino-1-benzyl-3-methyl-2-oxo-3H-pyrido[2,3-b][1,4]oxazine-7-carbonitrile (44 mg, 90.7%) as a white solid. LC/MS (ESI): mass calcd. for C₁₆H₁₄N₄O₂:294.1 m/z, found: 295.2 [M+H]⁺.

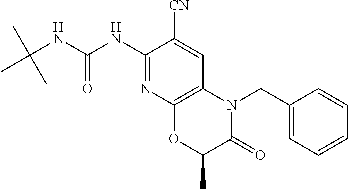 [SMILES: C[C@H]1Oc2nc(NC(=O)NC(C)(C)C)c(C#N)cc2N(Cc2ccccc2)C1=O]

### 1-[(3R)-1-benzyl-7-cyano-3-methyl-2-oxo-3H-pyrido[2,3-b][1,4]oxazin-6-yl]-3-tert-butylurea

To a stirred solution of (3R)-6-amino-1-benzyl-3-methyl-2-oxo-3H-pyrido[2,3-b][1,4]oxazine-7-carbonitrile (50 mg, 0.170 mmol, 1 equiv) and triphosgene (60.49 mg, 0.204 mmol, 1.2 equiv) in DCM (5 mL) was added TEA (51.57 mg, 0.510 mmol, 3 equiv) dropwise at 0° C. under nitrogen atmosphere. The resulting mixture was stirred for 30 min at 50° C. under nitrogen atmosphere. To the above mixture was added 2-methylpropan-2-amine (24.85 mg, 0.340 mmol, 2 equiv) dropwise at room temperature. The resulting mixture was stirred for additional 30 min at room temperature. The reaction was quenched with water (20 mL) and extracted with DCM (3×20 mL). The combined extracts were washed with water, brine, dried over Na₂SO₄, filtered and concentrated. The residue was purified by preparative HPLC using a YMC-Actus Triart Cis ExRS 150 mm×30 mm×5 μm column (eluent: 17% to 42% (v/v) CH₃CN and H₂O with 10 mmol/L NH₄HCO₃) to afford 1-[(3R)-1-benzyl-7-cyano-3-methyl-2-oxo-3H-pyrido[2,3-b][1,4]oxazin-6-yl]-3-tert-butylurea (2.4 mg, 3.55%) as a white solid. LC/MS (ESI): mass calcd. for C₂₁H₂₃N₅O₃:393.2 m/z, found: 394.20 [M+H]⁺. ¹H NMR (400 MHz, DMSO-d₆) δ 8.66 (s, 1H), 7.82 (s, 1H), 7.32-7.46 (m, 2H), 7.23-7.32 (m, 3H), 6.89 (s, 1H), 5.22-5.35 (m, 1H), 5.15 (s, 2H), 1.58 (d, J=6.8 Hz, 3H), 1.29 (s, 9H).

### Example 363: (S)-1-(tert-butyl)-3-(7-cyano-1-(1-(2,5-dichlorophenyl) ethyl)-2-oxo-1,2-dihydroquinoxalin-6-yl) urea

### Synthetic Scheme

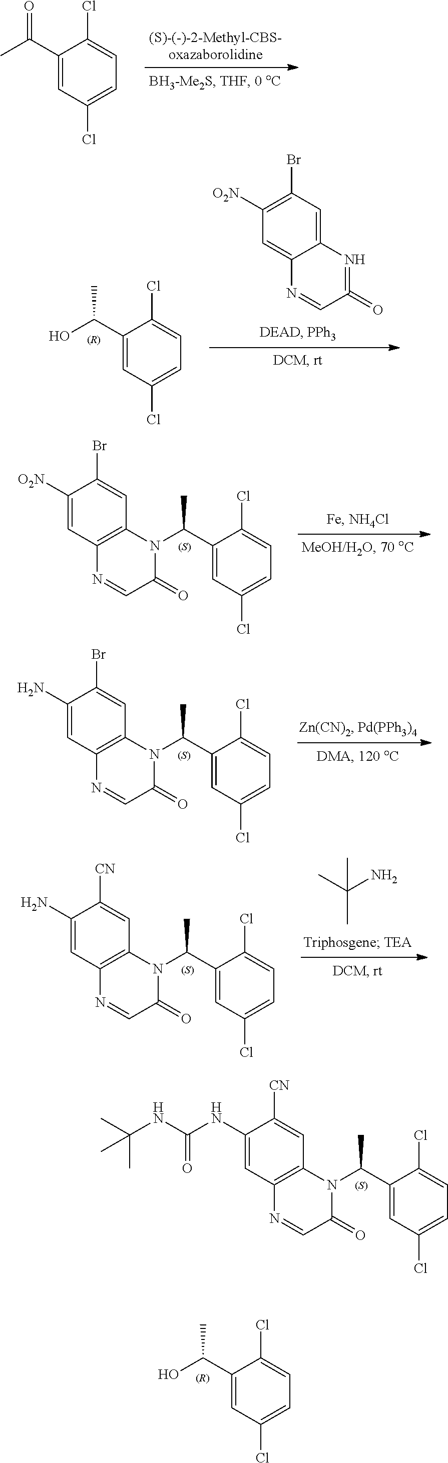

### (1R)-1-(2,5-dichlorophenyl) ethanol

To a stirred solution of CBS (0.56 g, 2.116 mmol, 0.2 equiv) in THF (10 mL) was added BH₃-Me₂S (0.53 g, 6.983 mmol, 0.66 equiv) and 1-(2,5-dichlorophenyl) ethanone (2 g, 10.580 mmol, 1 equiv) in portions at 0° C. under air atmosphere. The resulting mixture was stirred for 1 h at 0° C. under air atmosphere. The reaction was quenched with water at 0° C. The resulting mixture was extracted with EtOAc (3×30 mL). The combined organic layers were dried over anhydrous Na₂SO₄. After filtration, the filtrate was concentrated under reduced pressure. The residue was purified by silica gel column chromatography, eluted with PE/EA (1:1) to afford (1R)-1-(2,5-dichlorophenyl) ethanol (1.83 g, 90.54%) as a yellow oil. MS (ESI): mass calcd. For C₈H₈Cl₂O: 190.00 m/z, found 191.15[M+H]⁺.

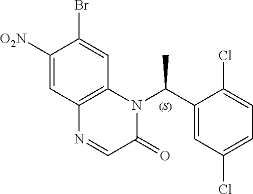

### 7-bromo-1-[(1S)-1-(2,5-dichlorophenyl) ethyl]-6-nitroquinoxalin-2-one

To a stirred solution of (1R)-1-(2,5-dichlorophenyl) ethanol (300 mg, 1.570 mmol, 1 equiv) and 7-bromo-6-nitro-1H-quinoxalin-2-one (508.85 mg, 1.884 mmol, 1.2 equiv) in DCM (5 mL) was added PPh₃ (617.80 mg, 2.355 mmol, 1.5 equiv) in portions at room temperature under nitrogen atmosphere. The resulting mixture was stirred for 30 min at room temperature under nitrogen atmosphere. To the above mixture was added DEAD (410.21 mg, 2.355 mmol, 1.5 equiv) dropwise over 1 min at 0° C. The resulting mixture was stirred for additional 1 h at room temperature. The resulting mixture was stirred for 1 h at room temperature under nitrogen atmosphere. The reaction was quenched with water at room temperature. The resulting mixture was extracted with CH₂Cl₂ (3×30 mL). The combined organic layers were dried over anhydrous Na₂SO₄. After filtration, the filtrate was concentrated under reduced pressure. The residue was purified by silica gel column chromatography, eluted with PE/EA (1:1) to afford 7-bromo-1-[(1S)-1-(2,5-dichlorophenyl) ethyl]-6-nitroquinoxalin-2-one (50 mg, 7.19%) as a yellow oil. MS (ESI): mass calcd. For C₁₆H₁₀BrCl₂N₃O₃: 440.93 m/z, found 443.85 [M+H, M+H+2]⁺.

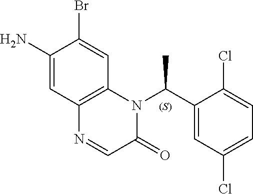

### 6-amino-7-bromo-1-[(1S)-1-(2,5-dichlorophenyl) ethyl]quinoxalin-2-one

A mixture of 7-bromo-1-[(1S)-1-(2,5-dichlorophenyl) ethyl]-6-nitroquinoxalin-2-one (220 mg, 0.497 mmol, 1 equiv), Fe (277.28 mg, 4.970 mmol, 10 equiv) and NH₄C₁ (265.59 mg, 4.970 mmol, 10 equiv) was added EtOH (10 mL) and H₂O (1 mL) and stirred at 70° C. for 1 h. The mixture was allowed to cool down to room temperature. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give 6-amino-7-bromo-1-[(1S)-1-(2,5-dichlorophenyl) ethyl]quinoxalin-2-one (200 mg, 97.51%) as a yellow solid. MS (ESI): mass calcd. for C₁₆H₁₂BrCl₂N₃O: 410.95 m/z, found 413.95 [M+H, M+H+2]⁺.

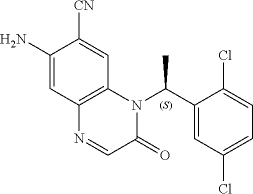

### 7-amino-4-[(1S)-1-(2,5-dichlorophenyl) ethyl]-3-oxoquinoxaline-6-carbonitrile

A mixture of 6-amino-7-bromo-1-[(1S)-1-(2,5-dichlorophenyl) ethyl]quinoxalin-2-one (200 mg, 0.484 mmol, 1 equiv), Zn(CN)₂ (68.22 mg, 0.581 mmol, 1.2 equiv) and Pd(PPh₃)₄ (55.95 mg, 0.048 mmol, 0.1 equiv) was treated with dimethylformamide (10 mL) and stirred at 110° C. under N₂ for 1 h. The mixture was allowed to cool down to room temperature. Quenched with water (50 mL) and extracted with DCM (3×50 mL). The combined extracts were washed with water, brine, dried over Na₂SO₄, filtered and concentrated. The residue was purified by column chromatography on silica gel with EA/PE (0-100%) to give 7-amino-4-[(1S)-1-(2,5-dichlorophenyl) ethyl]-3-oxoquinoxaline-6-carbonitrile (120 mg, 69.00%) as a white solid. MS (ESI): mass calcd. for C₁₇H₁₂C₁₂N₄O: 358.04 m/z, found 359.10 [M+H]⁺.

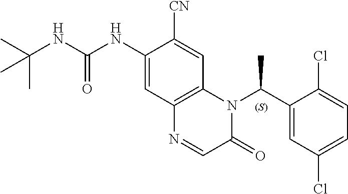

### 3-tert-butyl-1-{7-cyano-1-[(1S)-1-(2,5-dichlorophenyl) ethyl]-2-oxoquinoxalin-6-yl}urea

A solution of 7-amino-4-[(1S)-1-(2,5-dichlorophenyl) ethyl]-3-oxoquinoxaline-6-carbonitrile (20 mg, 0.056 mmol, 1 equiv) in DCM (5 mL) was treated with Triphosgene (16.52 mg, 0.056 mmol, 1 equiv) for 1 h at 0° C. under nitrogen atmosphere followed by the addition of TEA (28.17 mg, 0.280 mmol, 5 equiv) dropwise at 0° C. The resulting mixture was stirred for 1 h at room temperature under nitrogen atmosphere. To the above mixture was added erbumine (12.22 mg, 0.168 mmol, 3 equiv) dropwise over 1 min at room temperature. The resulting mixture was stirred for additional 1 h at room temperature. The reaction was quenched with water at room temperature. The resulting mixture was extracted with CH₂Cl₂ (3×10 mL). The combined organic layers were dried over anhydrous Na₂SO₄. After filtration, the filtrate was concentrated under reduced pressure. The residue was purified by preparative HPLC using a XBridge Prep OBD C18 Column 150 mm×30 mm×5 μm column (eluent: 44% to 74% (v/v) CH₃CN and H₂O with 10 mmol/L NH₄HCO_{3+0.05}% NH₃H₂O) to afford 3-tert-butyl-1-{7-cyano-1-[(1S)-1-(2,5-dichlorophenyl) ethyl]-2-oxoquinoxalin-6-yl}urea (1 mg, 3.89%) as a yellow solid. MS (ESI): mass calcd. for C₂₂H₂₁C₁₂N₅O₂: 457.11 m/z, found 458.15 [M+H]⁺. ¹H NMR (300 MHz, Acetonitrile-d₃) δ 8.45 (s, 1H), 8.12 (s, 1H), 7.83-7.98 (m, 2H), 7.28-7.45 (m, 2H), 7.15 (s, 1H), 5.92-6.20 (m, 1H), 5.75 (s, 1H), 1.90-1.95 (m, 3H), 1.38-1.41 (m, 9H).

### Example 364: 3-tert-butyl-1-{1-[(1S)-1-(5-chloro-2-fluorophenyl)ethyl]-7-cyano-2-oxoquinoxalin-6-yl}urea

### Synthetic Schemes

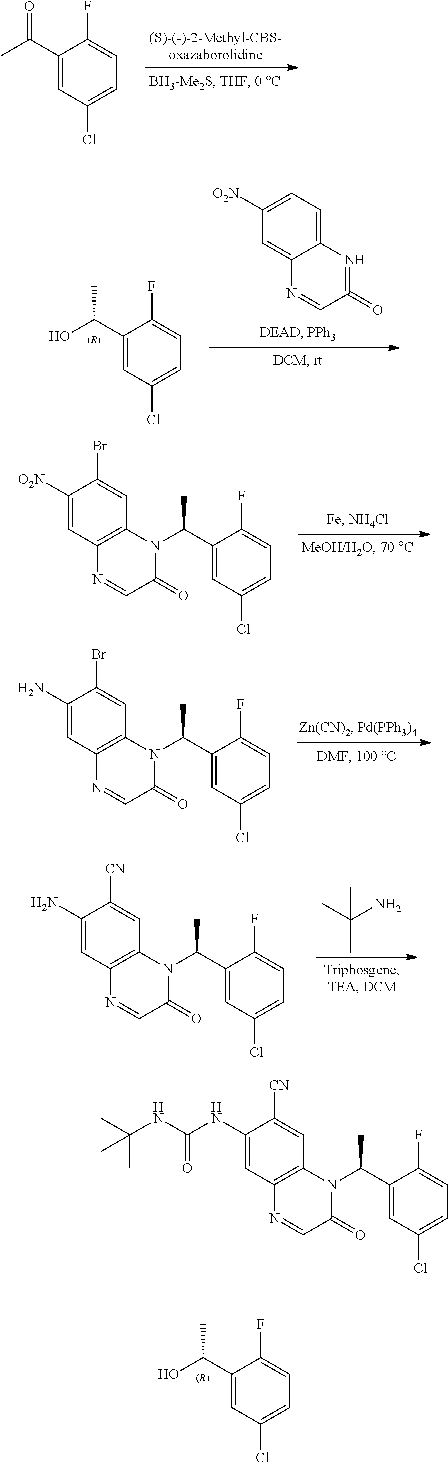

### (1R)-1-(5-chloro-2-fluorophenyl)ethanol

To a solution of (S)-(−)-2-Methyl-CBS-oxazaborolidine (1.22 g, 4.636 mmol, 0.4 equiv) in THF (24 mL) was added BH₃-Me₂S (1.16 g, 15.297 mmol, 1.32 equiv) and 1-(5-chloro-2-fluorophenyl)ethanone (2 g, 11.589 mmol, 1 equiv) at 0° C. The reaction was stirred at 0° C. for 2 h. TLC showed the desired product was generated. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give (1R)-1-(5-chloro-2-fluorophenyl)ethanol (1.4 g, 69.19%) as a white solid.

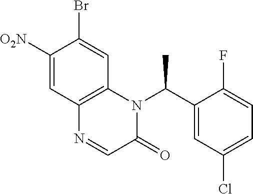 [SMILES: C[C@@H](c1cc(Cl)ccc1F)n1c(=O)cnc2cc([N+](=O)[O-])c(Br)cc21]

### 7-bromo-1-[(1S)-1-(5-chloro-2-fluorophenyl)ethyl]-6-nitroquinoxalin-2-one

To a solution of (1R)-1-(5-chloro-2-fluorophenyl)ethanol (970 mg, 5.556 mmol, 1 equiv) in DCM (12 mL) was added 7-bromo-6-nitro-1H-quinoxalin-2-one (1.50 g, 5.556 mmol, 1 equiv) and PPh₃ (2.19 g, 8.334 mmol, 1.5 equiv). After the mixture was stirred at rt for 1 h, to the above mixture was added DEAD (0.97 g, 5.556 mmol, 1 equiv) dropwise at 0° C. The resulting mixture was stirred for 2 h at 25° C. Quenched with water (50 mL) and extracted with DCM (3×50 mL). The combined extracts were washed with water, brine, dried over Na₂SO₄, filtered and concentrated. The residue was purified by column chromatography on silica gel with EA/PE (0-100%) to give 7-bromo-1-[(1S)-1-(5-chloro-2-fluorophenyl)ethyl]-6-nitroquinoxalin-2-one (140 mg, 5.91%) as a yellow oil. MS (ESI): mass calcd. for C₁₆H₁₀BrClFN₃O₃:424.96 m/z, found: 425.96, 427.96 [M+H, M+H+2]⁺.

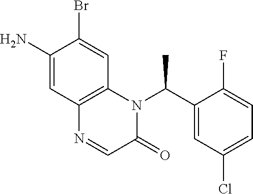 [SMILES: C[C@@H](c1cc(Cl)ccc1F)n1c(=O)cnc2cc(N)c(Br)cc21]

### 6-amino-7-bromo-1-[(1S)-1-(5-chloro-2-fluorophenyl)ethyl]quinoxalin-2-one

To a solution of 7-bromo-1-[(1S)-1-(5-chloro-2-fluorophenyl)ethyl]-6-nitroquinoxalin-2-one (190 mg, 0.445 mmol, 1 equiv) in MeOH (4 mL) and H₂O (0.4 mL) was added Fe (248.71 mg, 4.450 mmol, 10 equiv) and NH₄C₁ (238.22 mg, 4.450 mmol, 10 equiv). The reaction was stirred at 70° C. for 2 h. The mixture was allowed to cool down to room temperature and quenched with water (50 mL). The resulting mixture was filtered, the filter cake was washed with EA (50 mL). The filtrate was extracted with EA (3×50 mL). The combined extracts were washed with water, brine, dried over Na₂SO₄, filtered and concentrated. The residue was purified by column chromatography on silica gel with EA/PE (0-100%) to give 6-amino-7-bromo-1-[(1S)-1-(5-chloro-2-fluorophenyl)ethyl]quinoxalin-2-one (50 mg, 28.31%) as a yellow oil. MS (ESI): mass calcd. for C₁₆H₁₂BrClFN₃O:394.98 m/z, found: 395.98, 397.98 [M+H, M+H+2]*.

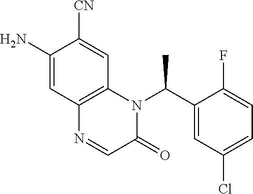 [SMILES: C[C@@H](c1cc(Cl)ccc1F)n1c(=O)cnc2cc(N)c(C#N)cc21]

### 7-amino-4-[(1S)-1-(5-chloro-2-fluorophenyl)ethyl]-3-oxoquinoxaline-6-carbonitrile

To a solution of 6-amino-7-bromo-1-[(1S)-1-(5-chloro-2-fluorophenyl)ethyl]quinoxalin-2-one (60 mg, 0.151 mmol, 1 equiv) in dimethylformamide (3 mL) was added Zn(CN)₂ (21.31 mg, 0.181 mmol, 1.2 equiv) and Pd(PPh₃)₄ (17.48 mg, 0.015 mmol, 0.1 equiv). The reaction was stirred at 100° C. for 2 h under N₂ atmosphere. The mixture was allowed to cool down to room temperature. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give 7-amino-4-[(1S)-1-(5-chloro-2-fluorophenyl)ethyl]-3-oxoquinoxaline-6-carbonitrile (30 mg, 57.86%) as a red solid. MS (ESI): mass calcd. for C₁₇H₁₂ClFN₄O:342.07 m/z, found: 343.07 [M+H]⁺.

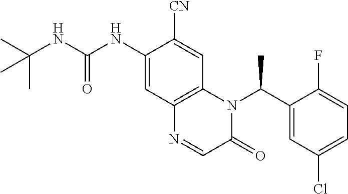 [SMILES: C[C@@H](c1cc(Cl)ccc1F)n1c(=O)cnc2cc(NC(=O)NC(C)(C)C)c(C#N)cc21]

### 3-tert-butyl-1-{1-[(1S)-1-(5-chloro-2-fluorophenyl)ethyl]-7-cyano-2-oxoquinoxalin-6-yl}urea

To a solution of 7-amino-4-[(1S)-1-(5-chloro-2-fluorophenyl)ethyl]-3-oxoquinoxaline-6-carbonitrile (30 mg, 0.088 mmol, 1 equiv) in DCM (5 mL) was added triphosgene (12.99 mg, 0.044 mmol, 0.5 equiv) at 0° C. under N₂ atmosphere. The resulting mixture was stirred for 1 h at 25° C. Then TEA (26.57 mg, 0.264 mmol, 3 equiv) was added into above system at 0° C. The resulting mixture was stirred 1 h at 25° C. To the above mixture was added erbumine (9.60 mg, 0.132 mmol, 1.5 equiv) dropwise at 0° C. The reaction was stirred at 25° C. for 2 h. Quenched with water (10 mL) and extracted with DCM (3×10 mL). The combined extracts were washed with water, brine, dried over Na₂SO₄, filtered and concentrated. The residue was purified by preparative HPLC using XBridge Prep OBD C18 30*150 mm, 5 μm column (eluent: 40% to 62% (v/v) CH₃CN and H₂O with 10 mmol/L NH₄HCO₃) to afford the title compound 3-tert-butyl-1-{1-[(1S)-1-(5-chloro-2-fluorophenyl)ethyl]-7-cyano-2-oxoquinoxalin-6-yl}urea (12.6 mg, 30.13%) as a yellow solid. MS (ESI): mass calcd. for C₂₂H₂₁ClFN₅O₂:441.14 m/z, found: 440.10 [M−H]⁻. ¹H NMR (300 MHz, DMSO-d₆) δ 8.47-8.38 (m, 2H), 8.25-8.38 (s, 2H), 7.67-7.76 (m, 1H), 7.35-7.46 (m, 1H), 7.07-7.19 (m, 1H), 6.95 (s, 1H), 6.29-6.37 (m, 1H), 1.83 (d, J=6.9 Hz, 3H), 1.36 (s, 9H). ¹⁹F NMR (282 MHz, DMSO) δ −118.07.

### Example 365: (S)-1-(tert-butyl)-3-(7-chloro-1-(1-(2,5-dichlorophenyl)ethyl)-2-oxo-1,2-dihydroquinoxalin-6-yl)urea

### Synthetic Scheme

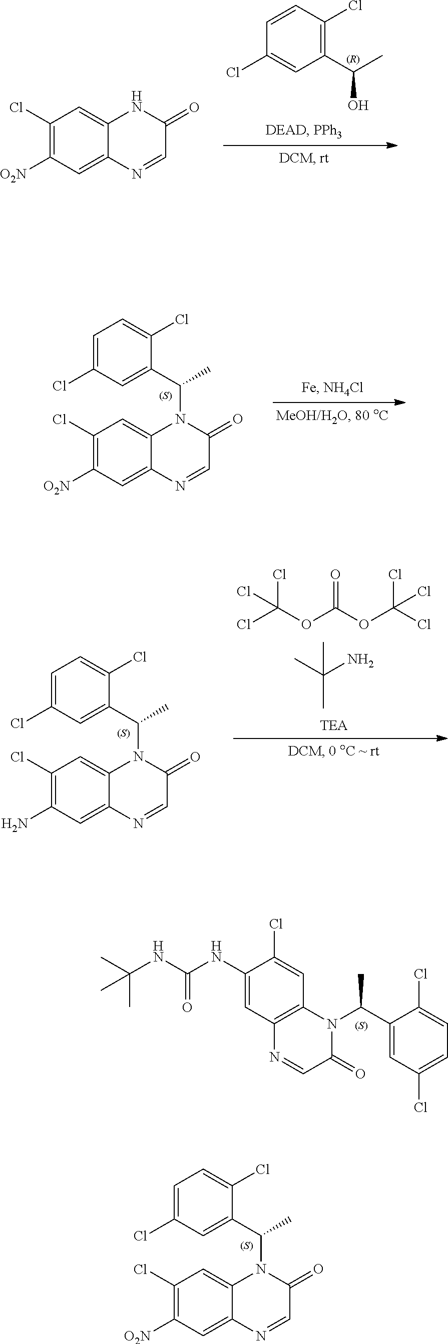

### (S)-7-chloro-1-(1-(2,5-dichlorophenyl)ethyl)-6-nitroquinoxalin-2(1H)-one

To a solution of 7-chloro-6-nitro-1H-quinoxalin-2-one (800 mg, 3.546 mmol, 1 equiv) in DCM (10 mL) was added (1R)-1-(2,5-dichlorophenyl)ethanol (1016.27 mg, 5.319 mmol, 1.5 equiv) and PPh₃ (1395.23 mg, 5.319 mmol, 1.5 equiv). The reaction was stirred at rt under N₂ for 0.5 h. To the above mixture was added DEAD (926.40 mg, 5.319 mmol, 1.5 equiv) under N₂ at 0° C. The resulting mixture was stirred for additional 1 h at 0° C. The reaction was monitored by TLC. Quenched with water (50 mL) and extracted with DCM (3×50 mL). The combined extracts were washed with water, brine, dried over Na₂SO₄, filtered and concentrated. The residue was purified by column chromatography on silica gel with EA/PE (0-100%) to give 7-chloro-1-[(1S)-1-(2,5-dichlorophenyl)ethyl]-6-nitroquinoxalin-2-one) as a yellow solid.

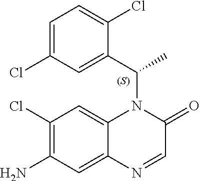

### (S)-6-amino-7-chloro-1-(1-(2,5-dichlorophenyl)ethyl)quinoxalin-2(1H)-one

To a solution of 7-chloro-1-[(1S)-1-(2,5-dichlorophenyl)ethyl]-6-nitroquinoxalin-2-one (60 mg, 0.151 mmol, 1 equiv) in MeOH (5 mL) and H₂O (0.5 mL) was added Fe (84.06 mg, 1.510 mmol, 10 equiv) and NH₄C₁ (80.51 mg, 1.510 mmol, 10 equiv). The reaction was stirred at 80° C. for 1 h. The mixture was allowed to cool down to rt. The resulting mixture was filtered, the filter cake was washed with MeOH (3×50 mL). The filtrate was concentrated under reduced pressure. Quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give 6-amino-7-chloro-1-[(1S)-1-(2,5-dichlorophenyl)ethyl]quinoxalin-2-one (30 mg, 54.07%) as a yellow solid. LC/MS: mass calcd. for C₁₆H₁₂Cl₃N₃O:367.00 m/z, found: 367.95 [M+H]⁺.

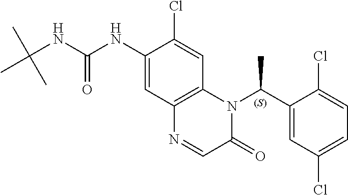

### (S)-1-(tert-butyl)-3-(7-chloro-1-(1-(2,5-dichlorophenyl)ethyl)-2-oxo-1,2-dihydroquinoxalin-6-yl)urea

To a solution of 6-amino-7-chloro-1-[(1S)-1-(2,5-dichlorophenyl)ethyl]quinoxalin-2-one (30 mg, 0.081 mmol, 1 equiv) in DCM (3 mL) was added triphosgene (24.15 mg, 0.081 mmol, 1 equiv) under N₂ at 0° C. The reaction was stirred at 0° C. under N₂ for 1 h. To the above mixture was added TEA (24.71 mg, 0.243 mmol, 3 equiv) at 0° C. The resulting mixture was stirred for additional 1 h at rt. Then, to the above mixture was added erbumine (17.86 mg, 0.243 mmol, 3 equiv). The resulting mixture was stirred for additional 1 h at rt. Quenched with water (10 mL) and extracted with DCM (3×10 mL). The combined extracts were washed with water, brine, dried over Na₂SO₄, filtered and concentrated. The residue was purified by preparative HPLC using a XBridge Shield RP18 OBD Column30*150 mm column (eluent: 51% to 67% (v/v) CH₃CN and Water (10 mmol/L NH₄HCO₃+0.1% NH₃·H₂O)) to afford the title compound 3-tert-butyl-1-{7-chloro-1-[(1S)-1-(2,5-dichlorophenyl)ethyl]-2-oxoquinoxalin-6-yl}urea (22 mg, 57.79%) as a yellow solid. LC/MS: mass calcd. for C₂₁H₂₁Cl₃N₄O₂:466.07 m/z, found: 467.10 [M+H]⁺. ¹H NMR (300 MHz, DMSO-d₆) δ 8.58 (s, 1H), 8.06 (d, J=9.7 Hz, 2H), 7.92 (s, 1H), 7.84 (d, J=2.2 Hz, 1H), 7.35-7.48 (m, 2H), 6.98 (s, 1H), 6.09 (d, J=7.0 Hz, 1H), 1.84 (d, J=6.8 Hz, 3H), 1.32 (s, 9H).

### Example 366: 1-(tert-butyl)-3-((R)-6-cyano-2-methyl-3-oxo-4-((S)-1-phenylethyl)-3,4-dihydro-2H-benzo[b][1,4]oxazin-7-yl)urea

### Synthetic Scheme

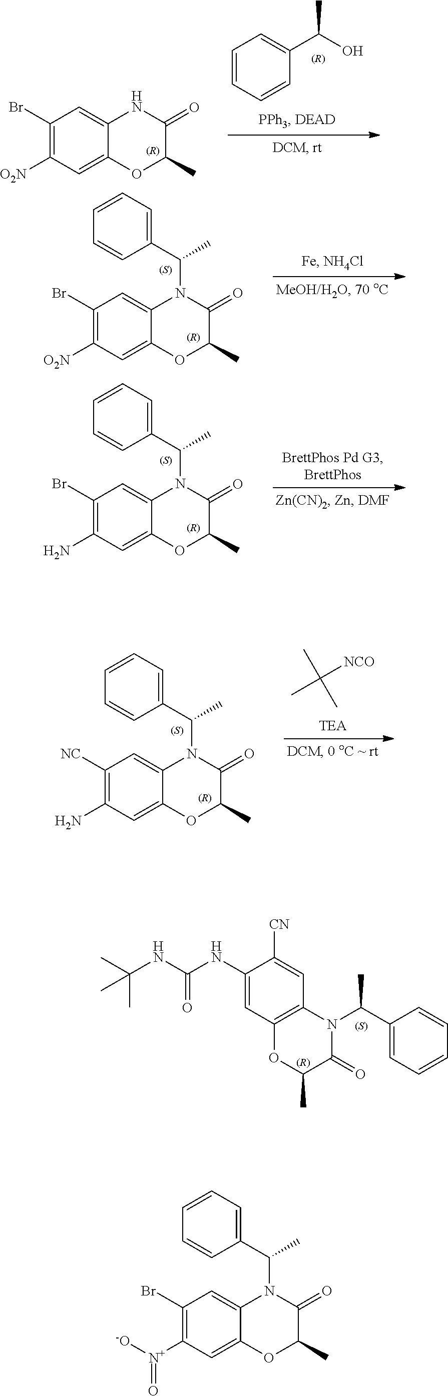

### (R)-6-bromo-2-methyl-7-nitro-4-((S)-1-phenylethyl)-2H-benzo[b][1,4]oxazin-3(4H)-one

To a stirred solution of (2R)-6-bromo-2-methyl-7-nitro-2,4-dihydro-1,4-benzoxazin-3-one (300 mg, 1.045 mmol, 1 equiv) and (R)-1-phenylethan-1-ol (127.67 mg, 1.045 mmol, 1 equiv) in DCM (4 mL) were added PPh₃ (411.16 mg, 1.567 mmol, 1.5 equiv) in portions at 0° C. under nitrogen atmosphere. The resulting mixture was stirred for 0.5 h at room temperature under nitrogen atmosphere. Then, to the above solution was added DEAD (546.00 mg, 3.135 mmol, 3 equiv) in portions at 0° C. under nitrogen atmosphere. The resulting mixture was stirred for 2 h at room temperature under nitrogen atmosphere. Desired product could be detected by LCMS. The reaction was quenched with water (15 mL). The resulting mixture was extracted with ethyl acetate (3×15 mL). The organic layers were combined, dried over anhydrous sodium sulfate, filtered and concentrated. The residue was purified by silica gel column chromatography, eluted with PE/EA (1:1) to afford (R)-6-bromo-2-methyl-7-nitro-4-((S)-1-phenylethyl)-2H-benzo[b][1,4]oxazin-3(4H)-one (225 mg, 55%) as a yellow oil. LC/MS: mass calcd for C₁₇H₁₅BrN₂O₄: 390.02, found: 391.05, 393.05 [M+H, M+H+2]⁺.

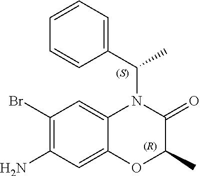

### (R)-7-amino-6-bromo-2-methyl-4-((S)-1-phenylethyl)-2H-benzo[b][1,4]oxazin-3(4H)-one

To a stirred solution of (R)-6-bromo-2-methyl-7-nitro-4-((S)-1-phenylethyl)-2H-benzo[b][1,4]oxazin-3(4H)-one (220 mg, 0.562 mmol, 1 equiv) and NH₄C₁ (300.80 mg, 5.620 mmol, 10 equiv) in MeOH/H₂O (10:1, 4 mL) was added Fe (314.04 mg, 5.620 mmol, 10 equiv) in portions at room temperature under air atmosphere. The resulting mixture was stirred for 2 h at 70° C. under air atmosphere. Desired product could be detected by LCMS. After cooling down to rt, the reaction was quenched with water (15 mL). The resulting mixture was extracted with ethyl acetate (3×15 mL). The organic layers were combined, dried over anhydrous sodium sulfate, filtered and concentrated. The residue was purified by silica gel column chromatography, eluted with PE/EA (1:1) to afford (R)-7-amino-6-bromo-2-methyl-4-((S)-1-phenylethyl)-2H-benzo[b][1,4]oxazin-3(4H)-one (190 mg, 70.15%) as a yellow solid. LC/MS: mass calcd for C₁₇H₁₇BrN₂O₂: 360.05, found: 361.02, 363.02 [M+H, M+H+2]*.

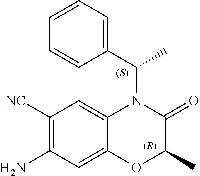

### (R)-7-amino-2-methyl-3-oxo-4-((S)-1-phenylethyl)-3,4-dihydro-2H-benzo[b][1,4]oxazine-6-carbonitrile

To a stirred solution of (R)-7-amino-6-bromo-2-methyl-4-((S)-1-phenylethyl)-2H-benzo[b][1,4]oxazin-3(4H)-one (190 mg, 0.526 mmol, 1 equiv) and BrettPhos Pd G3 (95.36 mg, 0.105 mmol, 0.2 equiv) in DMF (5 mL) were added BrettPhos (56.47 mg, 0.105 mmol, 0.2 equiv), Zn(CN)₂ (12.35 mg, 0.105 mmol, 0.2 equiv), and Zn (68.78 mg, 1.052 mmol, 2 equiv) in portions at room temperature under air atmosphere. The resulting mixture was stirred for 2 h at 120° C. under nitrogen atmosphere. Desired product could be detected by LCMS. After cooling down to rt, the reaction was quenched with water (15 mL). The resulting mixture was extracted with ethyl acetate (3×15 mL). The organic layers were combined, dried over anhydrous sodium sulfate, filtered and concentrated. The residue was purified by silica gel column chromatography, eluted with PE/EA (1:1) to afford (R)-7-amino-2-methyl-3-oxo-4-((S)-1-phenylethyl)-3,4-dihydro-2H-benzo[b][1,4]oxazine-6-carbonitrile (118 mg, 51.10%) as a yellow oil. LC/MS: mass for C₁₈H₁₇N₃O₂:307.13, found: 308.02 [M+H]⁺.

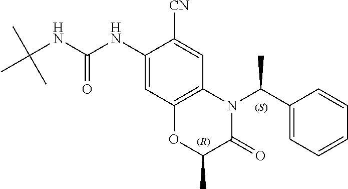

### 1-(tert-butyl)-3-((R)-6-cyano-2-methyl-3-oxo-4-((S)-1-phenylethyl)-3,4-dihydro-2H-benzo[b][1,4]oxazin-7-yl)urea

To a stirred solution of (R)-7-amino-2-methyl-3-oxo-4-((S)-1-phenylethyl)-3,4-dihydro-2H-benzo[b][1,4]oxazine-6-carbonitrile (108 mg, 0.351 mmol, 1 equiv) and 2-isocyanato-2-methylpropane (34.83 mg, 0.351 mmol, 1 equiv) in DCM (4 mL) was added TEA (106.67 mg, 1.053 mmol, 3 equiv) in portions at 0° C. under air atmosphere. The resulting mixture was stirred for 2 h at room temperature under air atmosphere. Desired product could be detected by LCMS. The reaction was quenched with water (15 mL). The resulting mixture was extracted with ethyl acetate (3×15 mL). The crude product (60 mg) was purified by Prep-HPLC with the following conditions (Column: XBridge Prep Cis OBD Column, 19*250 mm, 5 μm; Mobile Phase A: Water (10 mmol/L NH₄HCO₃), Mobile Phase B: ACN; Flow rate: 25 mL/min mL/min; Gradient: 50% B to 80% B in 7 min; Wave Length: 254 nm; RT1(min): 5.9; Number Of Runs: 2) to afford 1-(tert-butyl)-3-((R)-6-cyano-2-methyl-3-oxo-4-((S)-1-phenylethyl)-3,4-dihydro-2H-benzo[b][1,4]oxazin-7-yl)urea (37.7 mg, 26.39%) as a white solid. LC/MS: mass calcd for C₂₃H₂₆N₄O₃:406.20, found: 407.25 [M+H]⁺. ¹H NMR (400 MHz, DMSO-d₆) δ 8.25 (s, 1H), 7.84 (s, 1H), 7.43-7.33 (m, 3H), 7.20-7.33 (m, 3H), 6.97 (d, J=9.2 Hz, 2H), 5.95-6.14 (m, 1H), 1.78 (d, J=7.1 Hz, 3H), 1.48 (d, J=6.8 Hz, 3H), 1.28 (s, 9H).

### Example 367: 3-tert-butyl-1-[(2R)-6-cyano-4-[(2,5-dichlorophenyl)methyl]-2-methyl-3-oxo-2H-1,4-benzoxazin-7-yl]urea

### Synthetic Scheme

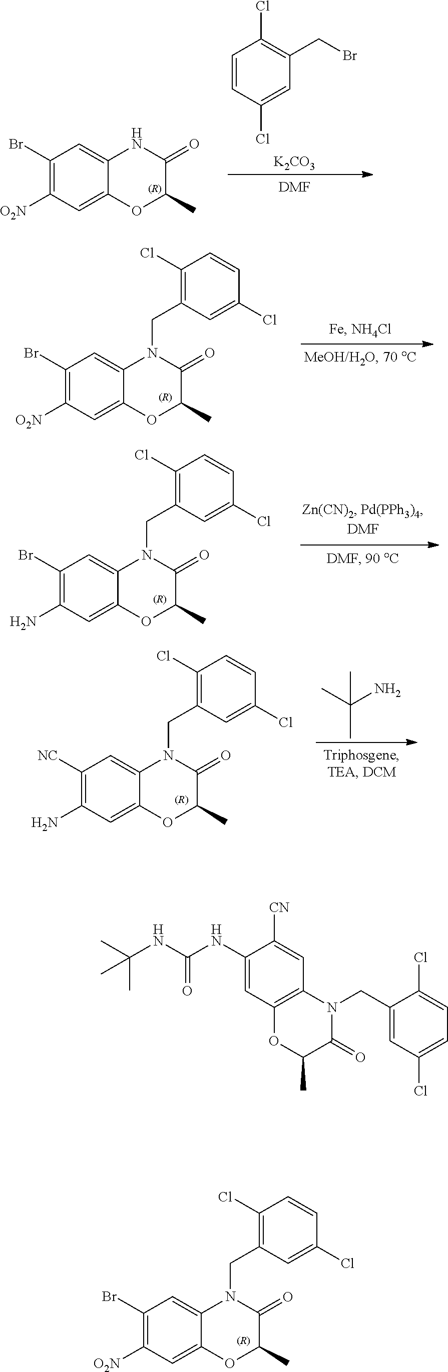

### (2R)-6-bromo-4-[(2,5-dichlorophenyl)methyl]-2-methyl-7-nitro-2H-1,4-benzoxazin-3-one

To a solution of (2R)-6-bromo-2-methyl-7-nitro-2,4-dihydro-1,4-benzoxazin-3-one (300 mg, 1.045 mmol, 1 equiv) in DMF (5 mL) was added 2-(bromomethyl)-1,4-dichlorobenzene (300.87 mg, 1.254 mmol, 1.2 equiv) and K₂CO₃ (436.45 mg, 3.135 mmol, 3 equiv). The reaction was stirred at 80° C. for 1 h. The mixture was allowed to cool down to room temperature. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-6-bromo-4-[(2,5-dichlorophenyl)methyl]-2-methyl-7-nitro-2H-1,4-benzoxazin-3-one (370 mg, 79.37%) as a white solid. MS (ESI): mass calcd. for C₁₆H₁₁BrCl₂N₂O₄:443.93 m/z, found: 444.92, 446.92 [M+H, M+H+2]⁺.

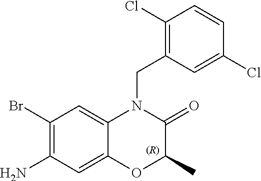

### (2R)-7-amino-6-bromo-4-[(2,5-dichlorophenyl)methyl]-2-methyl-2H-1,4-benzoxazin-3-one

To a solution of (2R)-6-bromo-4-[(2,5-dichlorophenyl)methyl]-2-methyl-7-nitro-2H-1,4-benzoxazin-3-one (370 mg, 0.829 mmol, 1 equiv) in MeOH (0.5 mL) and H₂O (5 mL) was added Fe (463.21 mg, 8.290 mmol, 10 equiv) and NH₄C₁ (443.67 mg, 8.290 mmol, 10 equiv). The reaction was stirred at 70° C. for 2 h. The mixture was allowed to cool down to room temperature and quenched with water (50 mL). The resulting mixture was filtered, the filter cake was washed with EA (50 mL). The filtrate was extracted with EA (3×50 mL). The combined extracts were washed with water, brine, dried over Na₂SO₄, filtered and concentrated. The residue was purified by column chromatography on silica gel with EA/PE (0-100%) to give (2R)-7-amino-6-bromo-4-[(2,5-dichlorophenyl)methyl]-2-methyl-2H-1,4-benzoxazin-3-one (270 mg, 78.23%) as a yellow oil. MS (ESI): mass calcd. for C₁₆H₁₃BrCl₂N₂O₂:413.95 m/z, found: 414.95, 416.95 [M+H, M+H+2]⁺.

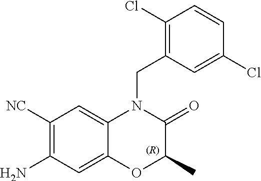

### (2R)-7-amino-4-[(2,5-dichlorophenyl)methyl]-2-methyl-3-oxo-2H-1,4-benzoxazine-6-carbonitrile

To a solution of (2R)-7-amino-6-bromo-4-[(2,5-dichlorophenyl)methyl]-2-methyl-2H-1,4-benzoxazin-3-one (260 mg, 0.625 mmol, 1 equiv) in DMF (5 mL) was added Zn(CN)₂ (88.04 mg, 0.750 mmol, 1.2 equiv) and Pd(PPh₃)₄ (72.21 mg, 0.063 mmol, 0.1 equiv). The reaction was stirred at 90° C. for 2 h under N₂ atmosphere. The mixture was allowed to cool down to room temperature. Quenched with water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-7-amino-4-[(2,5-dichlorophenyl)methyl]-2-methyl-3-oxo-2H-1,4-benzoxazine-6-carbonitrile (120 mg, 53.02%) as a white solid. MS (ESI): mass calcd. for C₁₇H₁₃Cl₂N₃O₂:361.04 m/z, found: 362.04 [M+H]⁺.

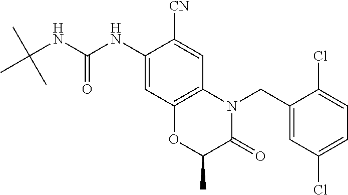 [SMILES: C[C@H]1Oc2cc(NC(=O)NC(C)(C)C)c(C#N)cc2N(Cc2cc(Cl)ccc2Cl)C1=O]

### 3-tert-butyl-1-[(2R)-6-cyano-4-[(2,5-dichlorophenyl)methyl]-2-methyl-3-oxo-2H-1,4-benzoxazin-7-yl]urea

To a solution of (2R)-7-amino-4-[(2,5-dichlorophenyl)methyl]-2-methyl-3-oxo-2H-1,4-benzoxazine-6-carbonitrile (110 mg, 0.304 mmol, 1 equiv) in DCM (10 mL) was added triphosgene (45.06 mg, 0.152 mmol, 0.5 equiv) at 0° C. under N₂ atmosphere. The resulting mixture was stirred for 1 h at 25° C. Then, TEA (92.19 mg, 0.912 mmol, 3 equiv) was added into above system at 0° C. The resulting mixture was stirred for 1 h at 25° C. To the above mixture was added erbumine (33.32 mg, 0.456 mmol, 1.5 equiv) dropwise at 0° C. The reaction was stirred at 25° C. for 2 h. Quenched with water (50 mL) and extracted with DCM (3×50 mL). The combined extracts were washed with water, brine, dried over Na₂SO₄, filtered and concentrated. The residue was purified by preparative HPLC using XBridge Prep OBD C18 30*150 mm, 5 μm column (eluent: 53% to 67% (v/v) CH₃CN and H₂O with 10 mmol/L NH₄HCO₃) to afford the title compound 3-tert-butyl-1-[(2R)-6-cyano-4-[(2,5-dichlorophenyl)methyl]-2-methyl-3-oxo-2H-1,4-benzoxazin-7-yl]urea (68.4 mg, 48.33%) as a white solid. MS (ESI): mass calcd. for C₂₂H₂₂C₁₂N₄O₃:460.11 m/z, found: 461.05 [M+H]⁺. ¹H NMR (300 MHz, DMSO-d₆) δ 8.31 (s, 1H), 7.88 (s, 1H), 7.53-7.62 (m, 1H), 7.37-7.47 (m, 1H), 7.32 (s, 1H), 7.03-7.11 (m, 1H), 6.98 (s, 1H), 4.96-5.25 (m, 3H), 1.52 (d, J=6.8 Hz, 3H), 1.30 (s, 9H).

### Example 368: (R)-1-(1-benzyl-3-methyl-2-oxo-7-(trifluoromethyl)-2,3-dihydro-1H-pyrido[2,3-b][1,4]oxazin-6-yl)-3-(tert-butyl)urea

### Synthetic Scheme

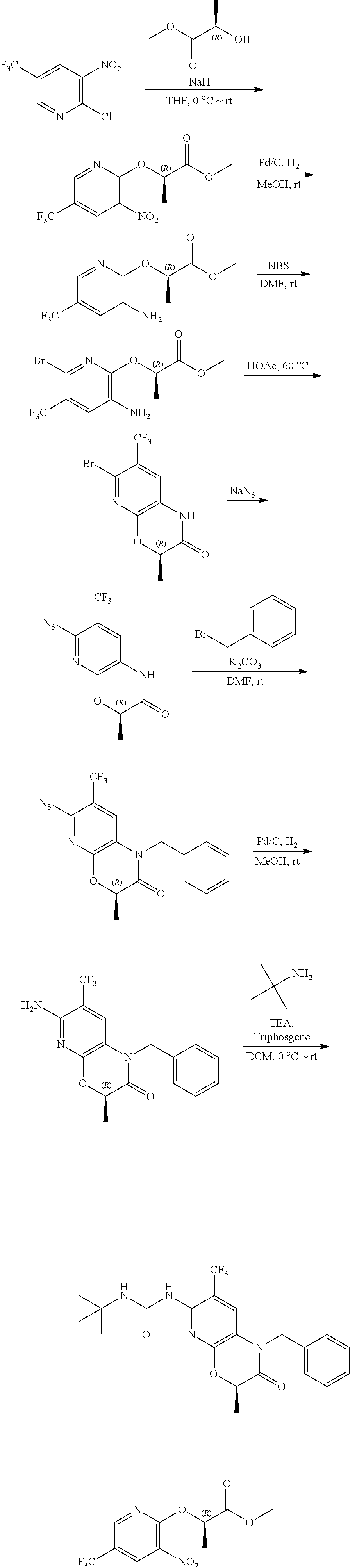

### methyl (R)-2-((3-nitro-5-(trifluoromethyl)pyridin-2-yl)oxy)propanoate

To a solution of methyl (R)-2-hydroxypropanoate (2.30 g, 22.071 mmol, 1 equiv) in THF (60 mL) was added NaH (794.50 mg, 33.107 mmol, 1.5 equiv) at 0° C. under air atmosphere. After 30 min, the 2-chloro-3-nitro-5-(trifluoromethyl)pyridine (5 g, 22.071 mmol, 1 equiv) was added to the mixture at 0° C. The resulting mixture was stirred for 2 h at room temperature. Desired product could be detected by LCMS. The mixture was diluted with water (150 mL). The resulting mixture was extracted with EA (3×150 mL). The combined organic layers were dried over anhydrous Na₂SO₄. After filtration, the filtrate was concentrated under reduced pressure. The residue obtained was purified by silica gel chromatography (0-20% ethyl acetate/petroleum ether) to afford the methyl (R)-2-((3-nitro-5-(trifluoromethyl)pyridin-2-yl)oxy)propanoate (3.5 g, 53.90%) as a white solid. MS (ESI): mass calcd. for C₁₀H₉F₃N₂O₅: 294.05 m/z, found 295.05 [M+H]⁺.

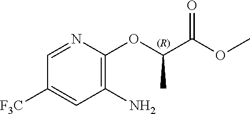

### (R)-2-((3-amino-5-(trifluoromethyl)pyridin-2-yl)oxy)propanoate

To a solution of methyl (R)-2-((3-nitro-5-(trifluoromethyl)pyridin-2-yl)oxy)propanoate (3.5 g, 11.897 mmol, 1 equiv) in MeOH (40 mL, 24.699 mmol) was added Pd/C (3.49 g) at 0° C. under N₂ atmosphere. The resulting mixture was maintained under hydrogen and stirred at 25° C. for 3 h. The resulting mixture was filtered, and the filter cake was washed with MeOH (4×40 mL). The filtrate was concentrated under reduced pressure. The residue obtained was purified by silica gel chromatography (0-50% ethyl acetate/petroleum ether) to afford the methyl (R)-2-((3-amino-5-(trifluoromethyl)pyridin-2-yl)oxy)propanoate (2.8 g, 89.08%) as a yellow oil. LC/MS: mass calcd. for C₁₀H₁₁F₃N₂O₃: 264.07, found: 265.05, 267.05 [M+H, M+H+2]⁺.

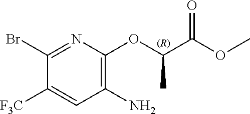

### methyl (R)-2-((3-amino-6-bromo-5-(trifluoromethyl)pyridin-2-yl)oxy)propanoate

To a stirred mixture of methyl (R)-2-((3-amino-5-(trifluoromethyl)pyridin-2-yl)oxy)propanoate (1.5 g, 5.677 mmol, 1 equiv) in DMF (15 mL) was added NBS (1.5 g, 8.515 mmol, 1.5 equiv) at −40° C. under air atmosphere. The resulting mixture was maintained under air and stirred at −40° C. for 3 h. The reaction was quenched with water (50 mL). The resulting mixture was extracted with ethyl acetate (3×50 mL). The organic layers were combined, dried over anhydrous Na₂SO₄, filtered and concentrated. The residue obtained was purified by silica gel chromatography (0-30% ethyl acetate/petroleum ether) to afford the methyl (R)-2-((3-amino-6-bromo-5-(trifluoromethyl)pyridin-2-yl)oxy)propanoate (530 mg, 27.21%) as a yellow oil. LC/MS: mass calcd. for C₁₀H₁₀BrF₃N₂O₃: 341.98, found: 342.90, 344.90 [M+H, M+H+2]⁺.

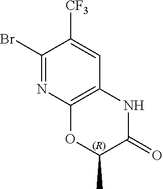

### (R)-6-bromo-3-methyl-7-(trifluoromethyl)-1H-pyrido[2,3-b][1,4]oxazin-2(3H)-one

A stirred mixture of methyl (R)-2-((3-amino-6-bromo-5-(trifluoromethyl)pyridin-2-yl)oxy)propanoate (700 mg, 2.040 mmol, 1 equiv) in HOAc (10 mL) was stirred at 80° C. for 3 h. After cooling down to room temperature, the reaction was quenched with NaHCO₃ solution (30 mL). The resulting mixture was extracted with ethyl acetate (3×30 mL). The organic layers were combined, dried over anhydrous Na₂SO₄, filtered and concentrated. The residue obtained was purified by silica gel chromatography (0-50% ethyl acetate/petroleum ether) to afford the (R)-6-bromo-3-methyl-7-(trifluoromethyl)-1H-pyrido[2,3-b][1,4]oxazin-2(3H)-one (370 mg, 58.30%) as a yellow oil. LC/MS: mass calcd. for C₉H₆BrF₃N₂O₂: 309.96, found: 310.85, 312.85 [M+H, M+H+2]⁺.

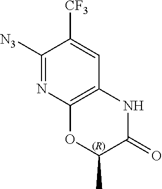

### (R)-6-azido-3-methyl-7-(trifluoromethyl)-1H-pyrido[2,3-b][1,4]oxazin-2(3H)-one

To a stirred mixture of (R)-6-bromo-3-methyl-7-(trifluoromethyl)-1H-pyrido[2,3-b][1,4]oxazin-2(3H)-one (300 mg, 0.964 mmol, 1 equiv) in DMF (5 mL) was added NaN₃ (94.05 mg, 1.446 mmol, 1.5 equiv) at 25° C. under air atmosphere. The resulting mixture was maintained under nitrogen and stirred at 110° C. for 3 h. After cooling down to room temperature, the reaction was quenched with water (20 mL). The resulting mixture was extracted with ethyl acetate (3×20 mL). The organic layers were combined, dried over anhydrous Na₂SO₄, filtered and concentrated. The residue obtained was purified by silica gel chromatography (0-50% ethyl acetate/petroleum ether) to afford the (R)-6-azido-3-methyl-7-(trifluoromethyl)-1H-pyrido[2,3-b][1,4]oxazin-2(3H)-one as a yellow oil. LC/MS: mass calcd. for C₉H₆F₃N₅O₂: 273.05, found: 274.10 [M+H]⁺.

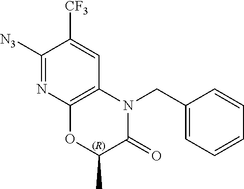

### (R)-6-azido-1-benzyl-3-methyl-7-(trifluoromethyl)-1H-pyrido[2,3-b][1,4]oxazin-2(3H)-one

To a stirred mixture of (R)-6-azido-3-methyl-7-(trifluoromethyl)-1H-pyrido[2,3-b][1,4]oxazin-2(3H)-one (147 mg, 0.538 mmol, 1 equiv) in DMF (5 mL) was added (bromomethyl)benzene (138.06 mg, 0.807 mmol, 1.5 equiv), K₂CO₃ (224.74 mg, 1.614 mmol, 3 equiv) at 25° C. under air atmosphere. The resulting mixture was maintained under air and stirred at 25° C. for 3 h. The reaction was quenched with water (10 mL). The resulting mixture was extracted with ethyl acetate (3×10 mL). The organic layers were combined, dried over anhydrous Na₂SO₄, filtered and concentrated. The residue obtained was purified by silica gel chromatography (0-50% ethyl acetate/petroleum ether) to afford the (R)-6-azido-1-benzyl-3-methyl-7-(trifluoromethyl)-1H-pyrido[2,3-b][1,4]oxazin-2(3H)-one (140 mg, 71.61%) as a yellow oil. LC/MS: mass calcd. for C₁₆H₁₂F₃N₅O₂: 363.09, found: 364.20 [M+H]⁺.

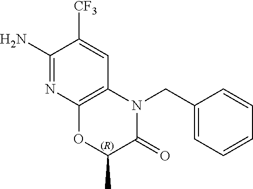

### (R)-6-amino-1-benzyl-3-methyl-7-(trifluoromethyl)-1H-pyrido[2,3-b][1,4]oxazin-2(3H)-one

To a stirred mixture of (R)-6-azido-1-benzyl-3-methyl-7-(trifluoromethyl)-1H-pyrido[2,3-b][1,4]oxazin-2(3H)-one (130 mg, 0.358 mmol, 1 equiv) in MeOH (3 mL) was added Pd/C (129.85 mg) at 0° C. under air atmosphere. The resulting mixture was maintained under hydrogen and stirred at 25° C. for 3 h. The resulting mixture was filtered, and the filter cake was washed with MeOH (4×40 mL). The filtrate was concentrated under reduced pressure. This afforded the (R)-6-amino-1-benzyl-3-methyl-7-(trifluoromethyl)-1H-pyrido[2,3-b][1,4]oxazin-2(3H)-one (110 mg, 91.14%) as a yellow oil. LC/MS: mass calcd. for C₁₆H₁₄F₃N₃O₂: 337.10, found: 338.15 [M+H]⁺.

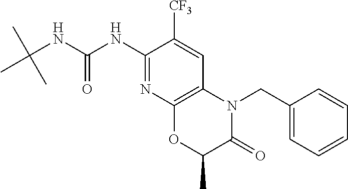 [SMILES: C[C@H]1Oc2nc(NC(=O)NC(C)(C)C)c(C(F)(F)F)cc2N(Cc2ccccc2)C1=O]

### (R)-1-(1-benzyl-3-methyl-2-oxo-7-(trifluoromethyl)-2,3-dihydro-1H-pyrido[2,3-b][1,4]oxazin-6-yl)-3-(tert-butyl)urea

To a stirred mixture of (R)-6-amino-1-benzyl-3-methyl-7-(trifluoromethyl)-1H-pyrido[2,3-b][1,4]oxazin-2(3H)-one (100 mg, 0.296 mmol, 1 equiv) in DCM (3 mL) was added triphosgene (87.97 mg, 0.296 mmol, 1 equiv) at 0 degrees C. under nitrogen atmosphere. After stirring at 0 degrees C. for 1 h, the TEA (150.00 mg, 1.480 mmol, 5 equiv) was added to the mixture at room temperature. After 30 min, erbumine (65.05 mg, 0.888 mmol, 3 equiv) and TEA (150.00 mg, 1.480 mmol, 5 equiv) was added to the reaction system. The resulting mixture was stirred for 1 h at 25 degrees C. Desired product could be detected by LCMS. The mixture was diluted with water (10 mL). The resulting mixture was extracted with EA (3×10 mL). The organic layers were combined, dried over anhydrous Na₂SO₄, filtered and concentrated. The residue was purified by preparative HPLC using a X Bridge Prep OBD C18 150 mm×30 mm×5 μm column (eluent: 49% to 71% (v/v) CH₃CN and H₂O with 10 mmol/L NH₄HCO₃) to afford the (R)-1-(1-benzyl-3-methyl-2-oxo-7-(trifluoromethyl)-2,3-dihydro-1H-pyrido[2,3-b][1,4]oxazin-6-yl)-3-(tert-butyl)urea (12.8 mg, 9.52%) as a white solid. MS (ESI): mass calcd. for C₂₁H₂₃F₃N₄O₃: 436.17 m/z, found 437.15 [M+H]⁺. ¹H-NMR (400 MHz, DMSO-d₆) δ 7.72 (s, 1H), 7.57 (s, 1H), 7.31-7.39 (m, 2H), 7.23-7.31 (m, 4H), 5.24-5.33 (m, 1H), 5.21 (d, J=7.5 Hz, 2H), 1.60 (d, J=6.8 Hz, 3H), 1.29 (s, 9H). ¹⁹F-NMR (376 MHz, DMSO) δ −58.98.

### Example 369: (R)-1-(4-benzyl-6-cyano-8-fluoro-2-methyl-3-oxo-3,4-dihydro-2H-benzo[b][1,4]oxazin-7-yl)-3-(tert-butyl)urea

### Synthetic Scheme

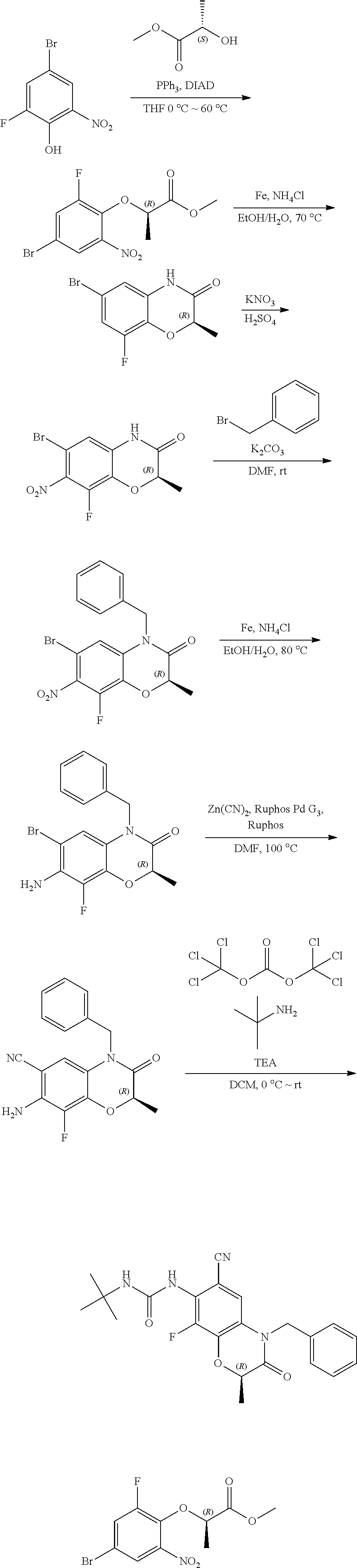

### methyl (R)-2-(4-bromo-2-fluoro-6-nitrophenoxy)propanoate

A solution of 4-bromo-2-fluoro-6-nitrophenol (5 g, 21.187 mmol, 1 equiv) in tetrahydrofuran (50 mL) was treated with methyl (2S)-2-hydroxypropanoate (2.21 g, 21.187 mmol, 1 equiv) and PPh₃ (8.34 g, 31.781 mmol, 1.5 equiv) for 1 h at rt under nitrogen atmosphere followed by the addition of DIAD (6.43 g, 31.781 mmol, 1.5 equiv) dropwise at 0° C. The resulting mixture was stirred for 2 h at rt under N₂ atmosphere. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give methyl (2R)-2-(4-bromo-2-fluoro-6-nitrophenoxy)propanoate (5.8 g, 84.99%) as a white solid. Desired product could be detected by TLC.

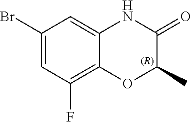

### (R)-6-bromo-8-fluoro-2-methyl-2H-benzo[b][1,4]oxazin-3(4H)-one

To a solution of methyl (2R)-2-(4-bromo-2-fluoro-6-nitrophenoxy)propanoate (5.8 g, 18.008 mmol, 1 equiv) in EtOH (60 mL)/H₂O (6 mL) was added Fe (10.06 g, 180.080 mmol, 10 equiv) and NH₄C₁ (9.63 g, 180.080 mmol, 10 equiv). The reaction was stirred at 70° C. for 3 h. The mixture was allowed to cool down to rt. Quenched with water (100 mL) and extracted with EA (3×100 mL). The combined extracts were washed with water, brine, dried over Na₂SO₄, filtered and concentrated. The residue was purified by column chromatography on silica gel with EA/PE (0-100%) to give (2R)-6-bromo-8-fluoro-2-methyl-2,4-dihydro-1,4-benzoxazin-3-one (4.5 g, 96.09%) as a white solid. MS (ESI): mass calcd. for C₉H₇BrFNO₂: 258.96 m/z, found 259.95, 261.95 [M+H, M+H+2]⁺.

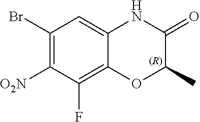

### (R)-6-bromo-8-fluoro-2-methyl-7-nitro-2H-benzo[b][1,4]oxazin-3(4H)-one

To a solution of (2R)-6-bromo-8-fluoro-2-methyl-2,4-dihydro-1,4-benzoxazin-3-one (1.5 g, 5.768 mmol, 1 equiv) in sulfuric acid (10 mL) was added KNO₃ (0.58 g, 5.768 mmol, 1 equiv) at 0° C. The reaction was stirred at 0° C. for 1 h. Quenched with ice water (50 mL) and extracted with EA (3×50 mL). The combined extracts were washed with water, brine, dried over Na₂SO₄, filtered and concentrated. The residue was purified by column chromatography on silica gel with EA/PE (0-100%) to give (2R)-6-bromo-8-fluoro-2-methyl-7-nitro-2,4-dihydro-1,4-benzoxazin-3-one (1.46 g, 82.98%) as a white solid. MS (ESI): mass calcd. for C₉H₆BrFN₂O₄: 303.95 m/z, found: 302.95, 304.95 [M−H, M−H+2]⁻.

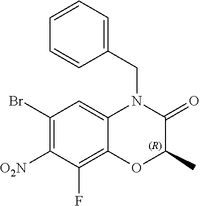

### (R)-4-benzyl-6-bromo-8-fluoro-2-methyl-7-nitro-2H-benzo[b][1,4]oxazin-3(4H)-one

To a solution of (2R)-6-bromo-8-fluoro-2-methyl-7-nitro-2,4-dihydro-1,4-benzoxazin-3-one (350 mg, 1.147 mmol, 1 equiv) in DMF (3 mL) was added benzyl bromide (294.35 mg, 1.720 mmol, 1.5 equiv) and K₂CO₃ (479.17 mg, 3.441 mmol, 3 equiv). The reaction was stirred at rt for 1 h. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give (2R)-4-benzyl-6-bromo-8-fluoro-2-methyl-7-nitro-2H-1,4-benzoxazin-3-one (416 mg, 91.75%) as a white solid. MS (ESI): mass calcd. for C₁₆H₁₂BrFN₂O₄: 394.00 m/z, found: 395.18, 397.18 [M+H, M+H+2]⁺.

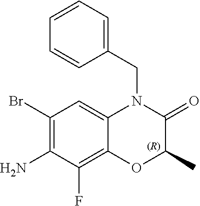

### (R)-7-amino-4-benzyl-6-bromo-8-fluoro-2-methyl-2H-benzo[b][1,4]oxazin-3(4H)-one

To a solution of (2R)-4-benzyl-6-bromo-8-fluoro-2-methyl-7-nitro-2H-1,4-benzoxazin-3-one (400 mg, 1.012 mmol, 1 equiv) in EtOH (8 mL)/H₂O (1 mL) was added Fe (565.26 mg, 10.120 mmol, 10 equiv) and NH₄C₁ (541.42 mg, 10.120 mmol, 10 equiv). The reaction was stirred at 70° C. for 3 h. The mixture was allowed to cool down to rt. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give (2R)-7-amino-4-benzyl-6-bromo-8-fluoro-2-methyl-2H-1,4-benzoxazin-3-one (250 mg, 67.63%) as a white solid. MS (ESI): mass calcd. for C₁₆H₁₄BrFN₂O₂: 364.02 m/z, found 365.05, 367.05 [M+H, M+H+2]⁺.

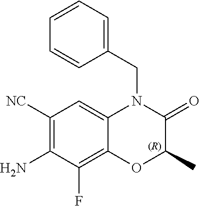

### (R)-7-amino-4-benzyl-8-fluoro-2-methyl-3-oxo-3,4-dihydro-2H-benzo[b][1,4]oxazine-6-carbonitrile

To a solution of (2R)-7-amino-4-benzyl-6-bromo-8-fluoro-2-methyl-2H-1,4-benzoxazin-3-one (240 mg, 0.657 mmol, 1 equiv) in DMF (5 mL) was added Zn(CN)₂ (154.33 mg, 1.314 mmol, 2 equiv), Zn (12.89 mg, 0.197 mmol, 0.3 equiv), BrettPhos (70.55 mg, 0.131 mmol, 0.2 equiv) and BrettPhos Pd G3 (119.15 mg, 0.131 mmol, 0.2 equiv). The reaction was stirred at 120° C. for 3 h under N₂ atmosphere. The mixture was allowed to cool down to rt. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give (2R)-7-amino-4-benzyl-8-fluoro-2-methyl-3-oxo-2H-1,4-benzoxazine-6-carbonitrile (160 mg, 78.21%) as a white solid. MS (ESI): mass calcd. for C₁₇H₁₄FN₃O₂: 311.11 m/z, found: 312.20 [M+H]⁺.

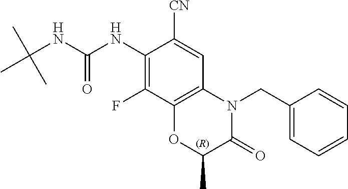

### (R)-1-(4-benzyl-6-cyano-8-fluoro-2-methyl-3-oxo-3,4-dihydro-2H-benzo[b][1,4]oxazin-7-yl)-3-(tert-butyl)urea

To a solution of (2R)-7-amino-4-benzyl-8-fluoro-2-methyl-3-oxo-2H-1,4-benzoxazine-6-carbonitrile (100 mg, 0.321 mmol, 1 equiv) in DCM (5 mL) was added triphosgene (95.31 mg, 0.321 mmol, 1 equiv). The reaction was stirred at 0° C. under N₂ for 1 h. The reaction was added TEA (162.52 mg, 1.605 mmol, 5 equiv) and stirred at 0° C. under N₂ for 1 h. The reaction was added erbumine (70.48 mg, 0.963 mmol, 3 equiv) and stirred at rt under N₂ for 1 h. Quenched with water (10 mL) and extracted with DCM (3×10 mL). The combined extracts were washed with water, brine, dried over Na₂SO₄, filtered and concentrated. The residue was purified by preparative HPLC using a Aeris PEPTIDE 5 um XB-C18 Axia 21.2 mm×250 mm, 5 μm column (eluent: 43% to 57% (v/v) CH₃CN and H₂O with 10 mmol/L NH₄HCO₃) to afford the title compound 1-[(2R)-4-benzyl-6-cyano-8-fluoro-2-methyl-3-oxo-2H-1,4-benzoxazin-7-yl]-3-tert-butylurea (42.1 mg, 31.66%) as a white solid. LC/MS (ESI): mass calcd. for C₂₂H₂₃FN₄O₃: 410.18 m/z, found: 411.15 [M+H]⁺. ¹H NMR (300 MHz, DMSO-d₆) δ 8.09 (s, 1H), 7.31-7.42 (m, 3H), 7.21-7.31 (m, 3H), 6.32 (s, 1H), 5.21 (s, 2H), 5.10-5.17 (m, 1H), 1.55-1.58 (d, J=9 Hz, 3H), 1.27 (s, 9H). ¹⁹F NMR (282 MHz, DMSO) δ −140.79.

### Example 370: (R)-1-(tert-butyl)-3-(6-cyano-4-(3-(difluoromethyl)benzyl)-8-fluoro-2-methyl-3-oxo-3,4-dihydro-2H-benzo[b][1,4]oxazin-7-yl)urea

### Synthetic Scheme

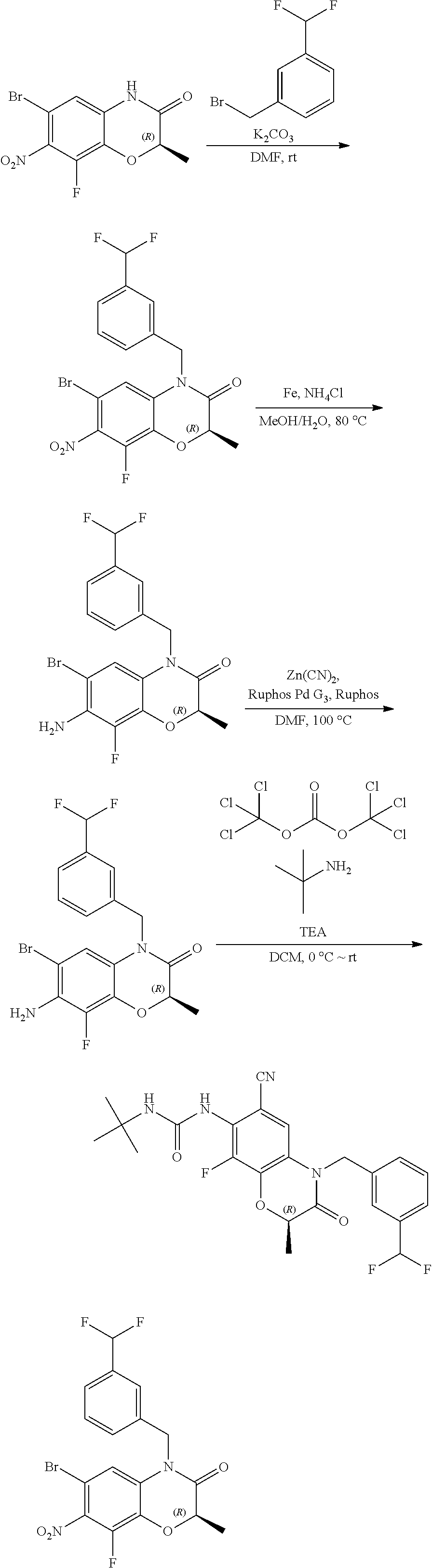

### (R)-6-bromo-4-(3-(difluoromethyl)benzyl)-8-fluoro-2-methyl-7-nitro-2H-benzo[b][1,4]oxazin-3(4H)-one

To a solution of (2R)-6-bromo-8-fluoro-2-methyl-7-nitro-2,4-dihydro-1,4-benzoxazin-3-one (300 mg, 0.983 mmol, 1 equiv) in DMF (5 mL) was added 1-(bromomethyl)-3-(difluoromethyl)benzene (326.07 mg, 1.474 mmol, 1.5 equiv) and K₂CO₃ (410.71 mg, 2.949 mmol, 3 equiv). The reaction was stirred at rt for 3 h. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give (2R)-6-bromo-4-{[3-(difluoromethyl)phenyl]methyl}-8-fluoro-2-methyl-7-nitro-2H-1,4-benzoxazin-3-one (400 mg, 91.36%) as a white solid. MS (ESI): mass calcd. for C₁₇H₁₂BrF₃N₂O₄: 443.99 m/z, found: 443.00, 445.00 [M−H, M−H+2]⁻.

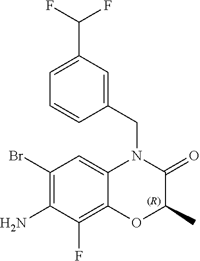

### (R)-7-amino-6-bromo-4-(3-(difluoromethyl)benzyl)-8-fluoro-2-methyl-2H-benzo[b][1,4]oxazin-3(4H)-one

To a solution of (2R)-6-bromo-4-{[3-(difluoromethyl)phenyl]methyl}-8-fluoro-2-methyl-7-nitro-2H-1,4-benzoxazin-3-one (600 mg, 1.348 mmol, 1 equiv) in EtOH (6 mL)/H₂O (0.6 mL) was added Fe (752.64 mg, 13.480 mmol, 10 equiv) and NH₄C₁ (720.90 mg, 13.480 mmol, 10 equiv). The reaction was stirred at 70° C. for 3 h. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give (2R)-7-amino-6-bromo-4-{[3-(difluoromethyl)phenyl]methyl}-8-fluoro-2-methyl-2H-1,4-benzoxazin-3-one (376 mg, 67.19%) as a white solid. MS (ESI): mass calcd. for C₁₇H₁₄BrF₃N₂O₂: 414.02 m/z, found 415.05, 417.05 [M+H, M+H+2]⁺.

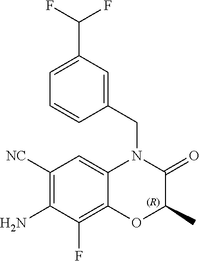

### (R)-7-amino-4-(3-(difluoromethyl)benzyl)-8-fluoro-2-methyl-3-oxo-3,4-dihydro-2H-benzo[b][1,4]oxazine-6-carbonitrile

To a solution of (2R)-7-amino-6-bromo-4-{[3-(difluoromethyl)phenyl]methyl}-8-fluoro-2-methyl-2H-1,4-benzoxazin-3-one (370 mg, 0.891 mmol, 1 equiv) in DMF (5 mL) was added Zn(CN)₂ (313.90 mg, 2.673 mmol, 3 equiv), Brettphos (95.67 mg, 0.178 mmol, 0.2 equiv), BrettPhos Pd G3 (140.23 mg, 0.178 mmol, 0.2 equiv), Zn (17.48 mg, 0.267 mmol, 0.3 equiv). The reaction was stirred at 120° C. for 3 h under N₂. The mixture was allowed to cool down to rt. Quenched with water (10 mL) and extracted with EA (3×10 mL). The combined extracts were washed with water, brine, dried over Na₂SO₄, filtered and concentrated. The residue was purified by column chromatography on silica gel with EA/PE (0-100%) to give (2R)-7-amino-4-{[3-(difluoromethyl)phenyl]methyl}-8-fluoro-2-methyl-3-oxo-2H-1,4-benzoxazine-6-carbonitrile (190 mg, 59.01%) as a white solid. MS (ESI): mass calcd. for C₁₈H₁₄F₃N₃O₂: 361.10 m/z, found: 360.00 [M−H]⁻.

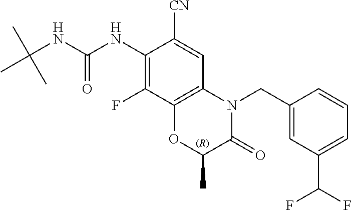

### (R)-1-(tert-butyl)-3-(6-cyano-4-(3-(difluoromethyl)benzyl)-8-fluoro-2-methyl-3-oxo-3,4-dihydro-2H-benzo[b][1,4]oxazin-7-yl)urea

To a solution of (2R)-7-amino-4-{[3-(difluoromethyl)phenyl]methyl}-8-fluoro-2-methyl-3-oxo-2H-1,4-benzoxazine-6-carbonitrile (100 mg, 0.277 mmol, 1 equiv) in DCM (5 mL) was added triphosgene (82.12 mg, 0.277 mmol, 1 equiv). The reaction was stirred at 0° C. under N₂ for 1 h. The reaction was added TEA (140.03 mg, 1.385 mmol, 5 equiv) and stirred at 0° C. under N₂ for 1 h. The reaction was added erbumine (60.73 mg, 0.831 mmol, 3 equiv) and stirred at rt under N₂ for 1 h. Quenched with water (10 mL) and extracted with DCM (3×10 mL). The combined extracts were washed with water, brine, dried over Na₂SO₄, filtered and concentrated. The residue was purified by preparative HPLC using a Aeris PEPTIDE 5 um XB-C18 Axia 21.2 mm×250 mm, 5 m column (eluent: 43% to 57% (v/v) CH₃CN and H₂O with 10 mmol/L NH₄HCO₃) to afford the title compound 3-tert-butyl-1-[(2R)-6-cyano-4-{[3-(difluoromethyl)phenyl]methyl}-8-fluoro-2-methyl-3-oxo-2H-1,4-benzoxazin-7-yl]urea (32.8 mg, 25.64%) as a white solid. LC/MS (ESI): mass calcd. for C₂₃H₂₃F₃N₄O₃: 460.17 m/z, found: 461.10 [M+H]⁺. ¹H NMR (300 MHz, DMSO-d₆) δ 8.10 (s, 1H), 7.35-7.57 (m, 5H), 7.03 (s, 1H), 6.32 (s, 1H), 5.27 (s, 2H), 5.12-5.18 (m, 1H), 1.56-1.58 (d, J=6 Hz, 3H), 1.27 (s, 9H). ¹⁹F NMR (282 MHz, DMSO) δ −109.47, −109.77, −140.68.

### Example 371: 3-tert-butyl-1-[(3R)-7-cyano-1-{[3-(difluoromethyl)phenyl]methyl}-3-methyl-2-oxo-3H-pyrido[2,3-b][1,4]oxazin-6-yl]urea

### Synthetic Scheme

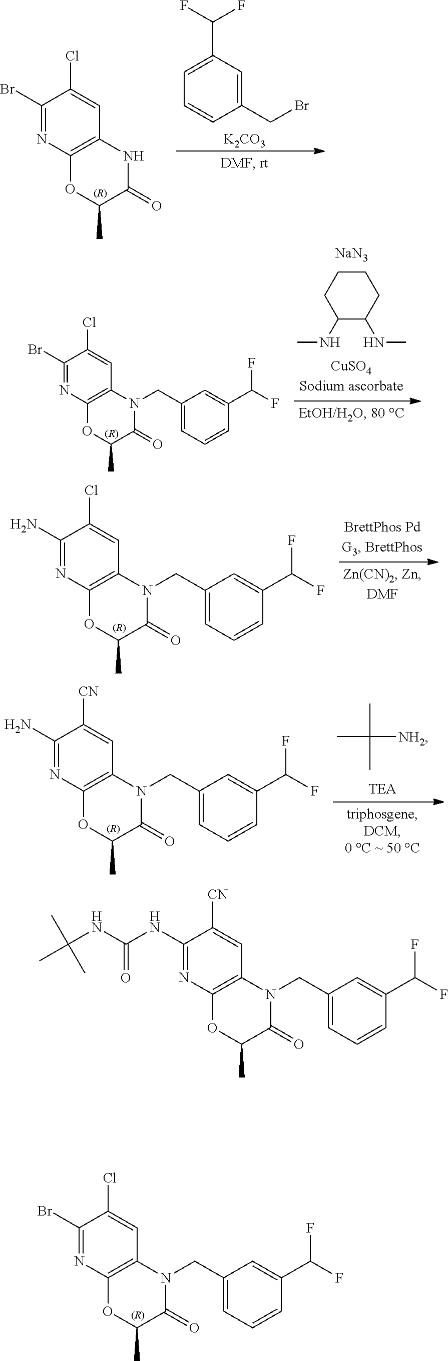

### (3R)-6-bromo-7-chloro-1-{[3-(difluoromethyl)phenyl]methyl}-3-methyl-3H-pyrido[2,3-b][1,4]oxazin-2-one

To a stirred mixture of (3R)-6-bromo-7-chloro-3-methyl-1H,3H-pyrido[2,3-b][1,4]oxazin-2-one (150 mg, 0.541 mmol, 1 equiv) and 1-(bromomethyl)-3-(difluoromethyl)benzene (238.97 mg, 1.082 mmol, 2 equiv) in DMF (10 mL) was added K₂CO₃ (225.75 mg, 1.623 mmol, 3 equiv) in portions at room temperature under air atmosphere. The resulting mixture was stirred for 4 h at 80° C. under air atmosphere. After cooling down to room temperature, the reaction was quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to afford (3R)-6-bromo-7-chloro-1-{[3-(difluoromethyl)phenyl]methyl}-3-methyl-3H-pyrido[2,3-b][1,4]oxazin-2-one (150 mg, 66.45%) as a white solid. LC/MS (ESI): mass calcd. for C₁₆H₁₂BrClF₂N₂O₂:416.0 m/z., found: 417.0, 419.0 [M+H, M+H+2]⁺.

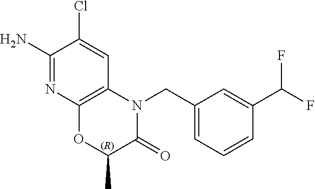

### (3R)-6-amino-7-chloro-1-{[3-(difluoromethyl)phenyl]methyl}-3-methyl-3H-pyrido[2,3-b][1,4]oxazin-2-one

To a stirred mixture of (3R)-6-bromo-7-chloro-1-{[3-(difluoromethyl)phenyl]methyl}-3-methyl-3H-pyrido[2,3-b][1,4]oxazin-2-one (150 mg, 0.359 mmol, 1 equiv) and NaN₃ (30.65 mg, 0.471 mmol, 3 equiv) in EtOH:H₂O=10:1 (5 mL) was added N1,N2-dimethylcyclohexane-1,2-diamine (30.65 mg, 0.215 mmol, 0.6 equiv) and CuSO₄ (25.09 mg, 0.157 mmol, 1 equiv) and sodium ascorbate (143.03 mg, 0.718 mmol, 2 equiv) in portions at room temperature under air atmosphere. The resulting mixture was stirred for 1 h at 80° C. under nitrogen atmosphere. After cooling down to room temperature, the reaction was quenched with water (20 mL) and extracted with EA (3×20 mL). The combined extracts were washed with water, brine, dried over Na₂SO₄, filtered and concentrated. The residue was purified by preparative HPLC using a YMC-Actus Triart Cis ExRS 150 mm×30 mm×5 μm column (eluent: 17% to 42% (v/v) CH₃CN and H₂O with 10 mmol/L NH₄HCO₃) to afford (3R)-6-amino-7-chloro-1-{[3-(difluoromethyl)phenyl]methyl}-3-methyl-3H-pyrido[2,3-b][1,4]oxazin-2-one (100 mg, 78.71%) as a white solid. LC/MS (ESI): mass calcd. for C₁₆H₁₄ClF₂N₃O₂:353.1 m/z, found: 354.1 [M+H]⁺.

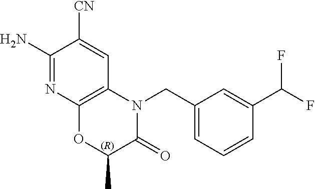

### (3R)-6-amino-1-{[3-(difluoromethyl)phenyl]methyl}-3-methyl-2-oxo-3H-pyrido[2,3-b][1,4]oxazine-7-carbonitrile

To a stirred mixture of (3R)-6-amino-7-chloro-1-{[3-(difluoromethyl)phenyl]methyl}-3-methyl-3H-pyrido[2,3-b][1,4]oxazin-2-one (100 mg, 0.283 mmol, 1 equiv) and Zn(CN)₂ (66.39 mg, 0.566 mmol, 2 equiv) in DMF (5 mL) was added BrettPhos Pd G3 (51.25 mg, 0.057 mmol, 0.2 equiv) and BrettPhos (17.67 mg, 0.033 mmol, 0.2 equiv) and Zn (5.54 mg, 0.085 mmol, 0.3 equiv) in portions at room temperature under air atmosphere. The resulting mixture was stirred for 1 h at 120° C. under nitrogen atmosphere. After cooling down to room temperature, the reaction was quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to afford (3R)-6-amino-1-{[3-(difluoromethyl)phenyl]methyl}-3-methyl-2-oxo-3H-pyrido[2,3-b][1,4]oxazine-7-carbonitrile (60 mg, 61.5%) as a white solid. LC/MS (ESI): mass calcd. for C₁₇H₁₄F₂N₄O₂:344.1 m/z, found: 345.1 [M+H]⁺.

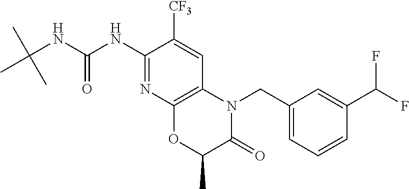 [SMILES: C[C@H]1Oc2nc(NC(=O)NC(C)(C)C)c(C(F)(F)F)cc2N(Cc2cccc(C(F)F)c2)C1=O]

### 3-tert-butyl-1-[(3R)-7-cyano-1-{[3-(difluoromethyl)phenyl]methyl}-3-methyl-2-oxo-3H-pyrido[2,3-b][1,4]oxazin-6-yl]urea

To a stirred solution of (3R)-6-amino-1-{[3-(difluoromethyl)phenyl]methyl}-3-methyl-2-oxo-3H-pyrido[2,3-b][1,4]oxazine-7-carbonitrile (60 mg, 0.174 mmol, 1 equiv) and triphosgene (62.05 mg, 0.209 mmol, 1.2 equiv) in DCM (5 mL) was added TEA (52.90 mg, 0.522 mmol, 3 equiv) dropwise at 0° C. under nitrogen atmosphere. The resulting mixture was stirred for 30 min at room temperature under nitrogen atmosphere. To the above mixture was added 2-methylpropan-2-amine (25.49 mg, 0.348 mmol, 2 equiv) dropwise at room temperature. The resulting mixture was stirred for additional 30 min at room temperature. The reaction was quenched with water (20 mL) and extracted with DCM (3×20 mL). The combined extracts were washed with water, brine, dried over Na₂SO₄, filtered and concentrated. The residue was purified by column chromatography on silica gel with EA/PE (0-100%) to afford 3-tert-butyl-1-[(3R)-7-cyano-1-{[3-(difluoromethyl)phenyl]methyl}-3-methyl-2-oxo-3H-pyrido[2,3-b][1,4]oxazin-6-yl]urea (2.8 mg, 3.51%) as a white solid. LC/MS (ESI): mass calcd. for C₂₂H₂₃F₂N₅O₃:443.2 m/z, found: 444.2 [M+H]⁺. ¹H NMR (400 MHz, DMSO-d₆) δ 8.64 (s, 1H), 7.85 (s, 1H), 7.32-7.45 (m, 4H), 6.85-7.20 (m, 2H), 5.25-5.34 (m, 1H), 5.20 (s, 2H), 1.58 (d, J=6.9 Hz, 3H), 1.29 (s, 9H). ¹⁹F NMR (376 MHz, DMSO) δ −109.69.

### Example 372: (S)-1-(tert-butyl)-3-(7-chloro-1-(1-(5-chloro-2-fluorophenyl)ethyl)-2-oxo-1,2-dihydroquinoxalin-6-yl)urea

### Synthetic Scheme

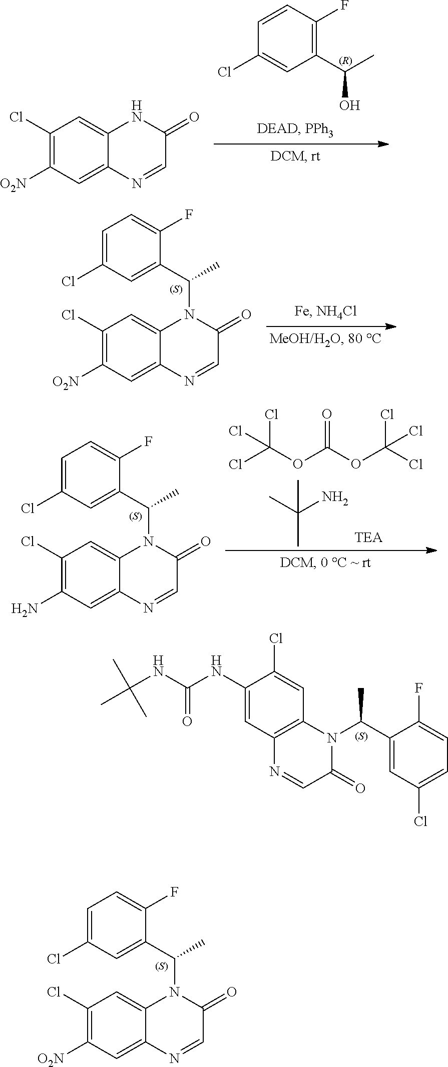

### (S)-7-chloro-1-(1-(5-chloro-2-fluorophenyl)ethyl)-6-nitroquinoxalin-2(1H)-one

To a solution of 7-chloro-6-nitro-1H-quinoxalin-2-one (500 mg, 2.216 mmol, 1 equiv) in DCM (10 mL) was added (1R)-1-(5-chloro-2-fluorophenyl)ethanol (386.99 mg, 2.216 mmol, 1 equiv) and PPh₃ (872.02 mg, 3.324 mmol, 1.5 equiv). The reaction was stirred at rt under N₂ for 0.5 h. To the above mixture was added DEAD (579.00 mg, 3.324 mmol, 1.5 equiv) at 0° C. The resulting mixture was stirred for additional 1 h at 0° C. Quenched with water (30 mL) and extracted with DCM (3×30 mL). The combined extracts were washed with water, brine, dried over Na₂SO₄, filtered and concentrated. The residue was purified by column chromatography on silica gel with EA/PE (0-100%) to give 7-chloro-1-[(1S)-1-(5-chloro-2-fluorophenyl)ethyl]-6-nitroquinoxalin-2-one (120 mg, 14.17%) as a yellow solid. LC/MS: mass calcd. for C₁₆H₁₀Cl₂FN₃O₃:381.01 m/z, found: 382.10 [M+H]⁺.

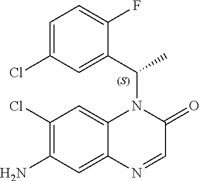

### (S)-6-amino-7-chloro-1-(1-(5-chloro-2-fluorophenyl)ethyl)quinoxalin-2(1H)-one

To a solution of 7-chloro-1-[(1S)-1-(5-chloro-2-fluorophenyl)ethyl]-6-nitroquinoxalin-2-one (140 mg, 0.366 mmol, 1 equiv) in MeOH (10 mL) and H₂O (1 mL) was added Fe (204.58 mg, 3.660 mmol, 10 equiv) and NH₄C₁ (195.95 mg, 3.660 mmol, 10 equiv). The reaction was stirred at 80° C. for 1 h. The mixture was allowed to cool down to rt. The resulting mixture was filtered, the filter cake was washed with MeOH (3×50 mL). The filtrate was concentrated under reduced pressure. Quenched with water (20 mL) and extracted with EA (3×20 mL). The combined extracts were washed with water, brine, dried over Na₂SO₄, filtered and concentrated. The residue was purified by column chromatography on silica gel with EA/PE (0-100%) to give 6-amino-7-chloro-1-[(1S)-1-(5-chloro-2-fluorophenyl)ethyl]quinoxalin-2-one (120 mg, 93.01%) as a yellow solid. LC/MS: mass calcd. for C₁₆H₁₂Cl₂FN₃O: 351.03 m/z, found: 352.00 [M+H]⁺.

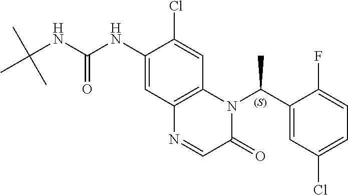

### (S)-1-(tert-butyl)-3-(7-chloro-1-(1-(5-chloro-2-fluorophenyl)ethyl)-2-oxo-1,2-dihydroquinoxalin-6-yl)urea

To a solution of 6-amino-7-chloro-1-[(1S)-1-(5-chloro-2-fluorophenyl)ethyl]quinoxalin-2-one (100 mg, 0.284 mmol, 1 equiv) in DCM (5 mL) was added triphosgene (84.25 mg, 0.284 mmol, 1 equiv) under N₂ at 0° C. The reaction was stirred at 0° C. under N₂ for 1 h. To the above mixture was added TEA (86.20 mg, 0.852 mmol, 3 equiv) at 0° C. The resulting mixture was stirred for additional 1 h at rt. Then, to the above mixture was added erbumine (62.30 mg, 0.852 mmol, 3 equiv). The resulting mixture was stirred for additional 1 h at rt. Quenched with water (20 mL) and extracted with DCM (3×20 mL). The combined extracts were washed with water, brine, dried over Na₂SO₄, filtered and concentrated. The residue was purified by preparative HPLC using a XBridge Prep OBD C18 Column, 30*150 mm, 5 μm column (eluent: 43% to 73% (v/v) CH₃CN and Water (10 mmol/L NH₄HCO_{3+0.05}% NH₃H₂O)) to afford the title compound 3-tert-butyl-1-{7-chloro-1-[(1S)-1-(5-chloro-2-fluorophenyl)ethyl]-2-oxoquinoxalin-6-yl}urea (48.8 mg, 37.94%) as a yellow solid. LC/MS: mass calcd. for C₂₁H₂₁Cl₂FN₄O₂:450.10 m/z, found: 451.15 [M+H]⁺. ¹H NMR (400 MHz, DMSO-d₆) δ 8.59 (s, 1H), 8.11 (s, 1H), 8.05 (s, 1H), 7.90 (s, 1H), 7.73 (dd, J=6.8, 2.7 Hz, 1H), 7.43 (ddd, J=8.7, 4.2, 2.6 Hz, 1H), 7.15 (dd, J=10.5, 8.8 Hz, 1H), 6.99 (s, 1H), 6.36 (s, 1H), 1.84 (d, J=6.8 Hz, 3H), 1.32 (s, 9H). ¹⁹F NMR (376 MHz, DMSO) δ(ppm): −118.1424.

### Example 373: 3-tert-butyl-1-[(2R)-4-[(5-chloro-2-fluorophenyl)methyl]-6-cyano-2-methyl-3-oxo-2H-1,4-benzoxazin-7-yl]urea

### Synthetic Scheme

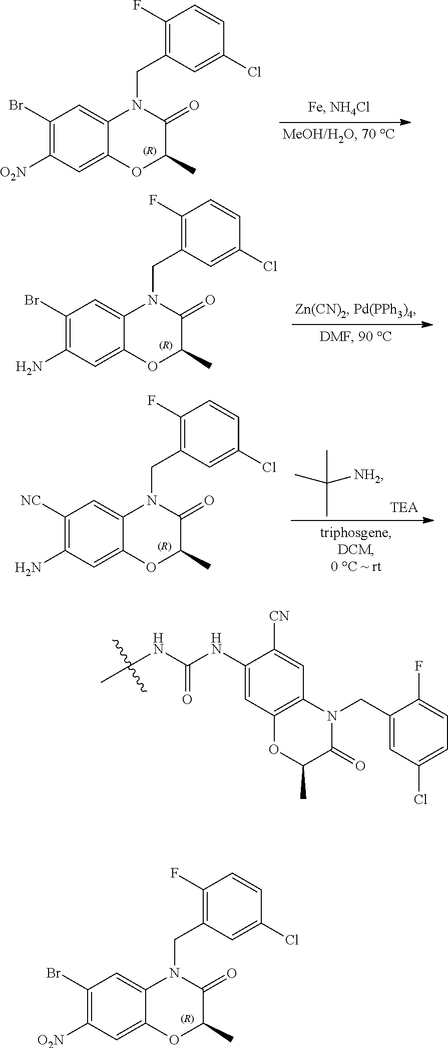

### (2R)-7-amino-6-bromo-4-[(5-chloro-2-fluorophenyl)methyl]-2-methyl-2H-1,4-benzoxazin-3-one

To a stirred mixture of (2R)-6-bromo-4-[(5-chloro-2-fluorophenyl)methyl]-2-methyl-7-nitro-2H-1,4-benzoxazin-3-one (300 mg, 0.698 mmol, 1 equiv) and NH₄C₁ (373.51 mg, 6.980 mmol, 10 equiv) in MeOH:H₂O=10:1 (10 mL) was added Fe (389.95 mg, 6.980 mmol, 10 equiv) in portions at room temperature under air atmosphere. The resulting mixture was stirred for 2 h at 70° C. under air atmosphere. After cooling down to room temperature, the reaction was quenched with water (30 mL) and extracted with EA (3×30 mL). The combined extracts were washed with water, brine, dried over Na₂SO₄, filtered and concentrated. The residue was purified by column chromatography on silica gel with EA/PE (0-100%) to afford (2R)-7-amino-6-bromo-4-[(5-chloro-2-fluorophenyl)methyl]-2-methyl-2H-1,4-benzoxazin-3-one (236 mg, 84.57%) as a white solid. LC/MS (ESI): mass calcd. for C₁₆H₁₃BrClFN₂O₂:398.0 m/z, found: 399.0, 401.0 [M+H, M+H+2]⁺.

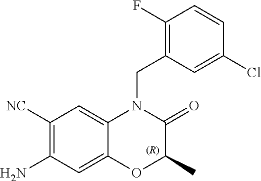

### (2R)-7-amino-4-[(5-chloro-2-fluorophenyl)methyl]-2-methyl-3-oxo-2H-1,4-benzoxazine-6-carbonitrile

To a stirred mixture of (2R)-7-amino-6-bromo-4-[(5-chloro-2-fluorophenyl)methyl]-2-methyl-2H-1,4-benzoxazin-3-one (230 mg, 0.576 mmol, 1 equiv) and Zn(CN)₂ (81.09 mg, 0.691 mmol, 1.2 equiv) in DMF (10 mL) was added Pd(PPh₃)₄ (66.51 mg, 0.058 mmol, 0.1 equiv) in portions at room temperature under air atmosphere. The resulting mixture was stirred for 2 h at 90° C. under nitrogen atmosphere. After cooling down to room temperature, the reaction was quenched with water (30 mL) and extracted with EA (3×30 mL). The combined extracts were washed with water, brine, dried over Na₂SO₄, filtered and concentrated. TLC showed the desired product was generated. The residue was purified by column chromatography on silica gel with EA/PE (0-100%) to afford (2R)-7-amino-4-[(5-chloro-2-fluorophenyl)methyl]-2-methyl-3-oxo-2H-1,4-benzoxazine-6-carbonitrile (111 mg, 55.78%) as a white solid.

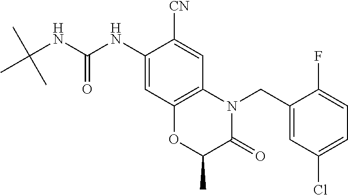 [SMILES: C[C@H]1Oc2cc(NC(=O)NC(C)(C)C)c(C#N)cc2N(Cc2cc(Cl)ccc2F)C1=O]

### 3-tert-butyl-1-[(2R)-4-[(5-chloro-2-fluorophenyl)methyl]-6-cyano-2-methyl-3-oxo-2H-1,4-benzoxazin-7-yl]urea

To a stirred solution of (2R)-7-amino-4-[(5-chloro-2-fluorophenyl)methyl]-2-methyl-3-oxo-2H-1,4-benzoxazine-6-carbonitrile (60 mg, 0.174 mmol, 1 equiv) and triphosgene (61.79 mg, 0.209 mmol, 1.2 equiv) in DCM (5 mL) was added TEA (52.68 mg, 0.522 mmol, 3 equiv) dropwise at 0° C. under nitrogen atmosphere. The resulting mixture was stirred for 30 min at room temperature under nitrogen atmosphere. To the above mixture was added 2-methylpropan-2-amine (25.38 mg, 0.348 mmol, 2 equiv) dropwise at room temperature. The resulting mixture was stirred for additional 30 min at room temperature. The reaction was quenched with water (30 mL) and extracted with DCM (3×30 mL). The combined extracts were washed with water, brine, dried over Na₂SO₄, filtered and concentrated. The residue was purified by preparative HPLC using a YMC-Actus Triart Cis ExRS 150 mm×30 mm×5 μm column (eluent: 17% to 42% (v/v) CH₃CN and H₂O with 10 mmol/L NH₄HCO₃) to afford 3-tert-butyl-1-[(2R)-4-[(5-chloro-2-fluorophenyl)methyl]-6-cyano-2-methyl-3-oxo-2H-1,4-benzoxazin-7-yl]urea (26.1 mg, 33.81%) as a white solid. LC/MS (ESI): mass calcd. for C₂₂H₂₂C₁FN₄O₃:444.1 m/z, found: 445.15 [M+H]⁺. ¹H NMR (400 MHz, DMSO-d₆) δ 8.31 (s, 1H), 7.86 (s, 1H), 7.38-7.50 (m, 2H), 7.26-7.38 (m, 1H), 7.10-7.26 (m, 1H), 6.99 (s, 1H), 5.09-5.41 (m, 2H), 4.89-5.08 (m, 1H), 1.49 (d, J=6.8 Hz, 3H), 1.29 (s, 9H). ¹⁹F NMR (376 MHz, DMSO) δ −119.29.

### Example 374: (R)-1-(tert-butyl)-3-(1-(3-(difluoromethyl) benzyl)-3-methyl-2-oxo-7-(trifluoromethyl)-2,3-dihydro-1H-pyrido[2,3-b][1,4]oxazin-6-yl) urea

### Synthetic Scheme

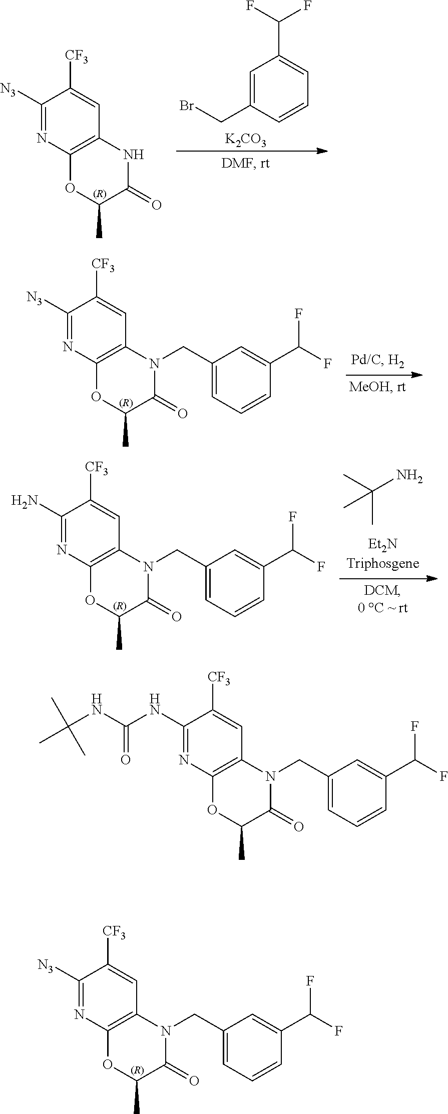

### (3R)-6-azido-1-{[3-(difluoromethyl) phenyl]methyl}-3-methyl-7-(trifluoromethyl)-3H-pyrido[2,3-b][1,4]oxazin-2-one

To a stirred solution of (3R)-6-azido-3-methyl-7-(trifluoromethyl)-1H,3H-pyrido[2,3-b][1,4]oxazin-2-one (80 mg, 0.293 mmol, 1 equiv) and 1-(bromomethyl)-3-(difluoromethyl) benzene (77.68 mg, 0.352 mmol, 1.2 equiv) in DMF (5 mL) was added K₂CO₃ (122.31 mg, 0.879 mmol, 3 equiv) in portions at room temperature under air atmosphere. The resulting mixture was stirred for 2 h at room temperature under air atmosphere. The reaction was quenched with water at room temperature. The resulting mixture was extracted with EtOAc (3×30 mL). The combined organic layers were dried over anhydrous Na₂SO₄. After filtration, the filtrate was concentrated under reduced pressure. The residue was purified by silica gel column chromatography, eluted with PE/EA (1:1) to afford (3R)-6-azido-1-{[3-(difluoromethyl) phenyl]methyl}-3-methyl-7-(trifluoromethyl)-3H-pyrido[2,3-b][1,4]oxazin-2-one (100 mg, 82.62%) as a yellow oil. MS (ESI): mass calcd. for C₁₇H₁₂F₅N₅O₂: 413.09 m/z, found 414.05 [M+H]⁺.

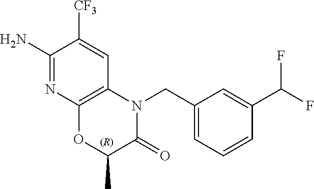

### (3R)-6-amino-1-{[3-(difluoromethyl) phenyl]methyl}-3-methyl-7-(trifluoromethyl)-3H-pyrido[2,3-b][1,4]oxazin-2-one

A mixture of (3R)-6-azido-1-{[3-(difluoromethyl) phenyl]methyl}-3-methyl-7-(trifluoromethyl)-3H-pyrido[2,3-b][1,4]oxazin-2-one (100 mg, 0.242 mmol, 1 equiv) and Pd/C (30 mg, 0.282 mmol, 1.17 equiv) in MeOH (5 mL) was stirred for 1 h at room temperature under hydrogen atmosphere. The resulting mixture was filtered, and the filter cake was washed with MeOH (4×10 mL). The filtrate was concentrated under reduced pressure. The residue was purified by silica gel column chromatography, eluted with PE/EA (1:1) to afford (3R)-6-amino-1-{[3-(difluoromethyl) phenyl]methyl}-3-methyl-7-(trifluoromethyl)-3H-pyrido[2,3-b][1,4]oxazin-2-one (80 mg, 85.37%) as a yellow oil. MS (ESI): mass calcd. for C₁₇H₁₄F₅N₃O₂: 387.10 m/z, found 388.05 [M+H]⁺.

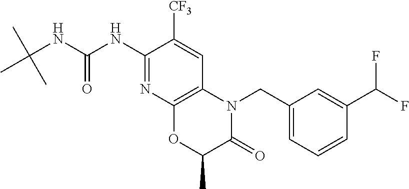 [SMILES: C[C@H]1Oc2nc(NC(=O)NC(C)(C)C)c(C(F)(F)F)cc2N(Cc2cccc(C(F)F)c2)C1=O]

### (R)-1-(tert-butyl)-3-(1-(3-(difluoromethyl) benzyl)-3-methyl-2-oxo-7-(trifluoromethyl)-2,3-dihydro-1H-pyrido[2,3-b][1,4]oxazin-6-yl) urea

A solution of (3R)-6-amino-1-{[3-(difluoromethyl) phenyl]methyl}-3-methyl-7-(trifluoromethyl)-3H-pyrido[2,3-b][1,4]oxazin-2-one (80 mg, 0.207 mmol, 1 equiv) in toluene (5 mL) was treated with Triphosgene (122.58 mg, 0.414 mmol, 2 equiv) for 30 min at room temperature under nitrogen atmosphere followed by the addition of triethylamine (104.51 mg, 1.035 mmol, 5 equiv) in portions at 0° C. The resulting mixture was stirred for 2 h at 90° C. under nitrogen atmosphere. To the above mixture was added erbumine (45.32 mg, 0.621 mmol, 3 equiv) dropwise over 1 min at room temperature. The resulting mixture was stirred for additional 1 h at room temperature. The reaction was quenched with water at room temperature. The resulting mixture was extracted with EtOAc (3×30 mL). The combined organic layers were dried over anhydrous Na₂SO₄. After filtration, the filtrate was concentrated under reduced pressure. The residue was purified by preparative HPLC using a XSelect CSH Fluoro Pheny 250 mm×19 mm×5 m column (eluent: 45% to 75% (v/v) CH₃CN and H₂O with 10 mmol/L 0.05% TFA) to afford 3-tert-butyl-1-[(3R)-1-{[3-(difluoromethyl) phenyl]methyl}-3-methyl-2-oxo-7-(trifluoromethyl)-3H-pyrido[2,3-b][1,4]oxazin-6-yl]urea (30 mg, 29.38%) as a yellow solid. MS (ESI): mass calcd. for C₂₂H₂₃F₅N₄O₃: 486.17 m/z, found 487.20 [M+H]⁺. ¹H NMR (300 MHz, DMSO-d₆) δ 7.72 (s, 1H), 7.61 (s, 1H), 7.38-7.58 (m, 4H), 7.30 (s, 1H), 7.02 (s, 1H), 5.18-5.40 (m, 3H), 1.60 (d, J=6.8 Hz, 3H), 1.15-1.42 (m, 9H). ¹⁹F NMR (282 MHz, DMSO) δ −59.022, −109.803

### Example 375: 3-tert-butyl-1-{1-[(1S)-1-[2-fluoro-5-(2,2,2-trifluoro-1-hydroxyethyl)phenyl]ethyl]-2-oxoquinoxalin-6-yl}urea

### Synthetic Scheme

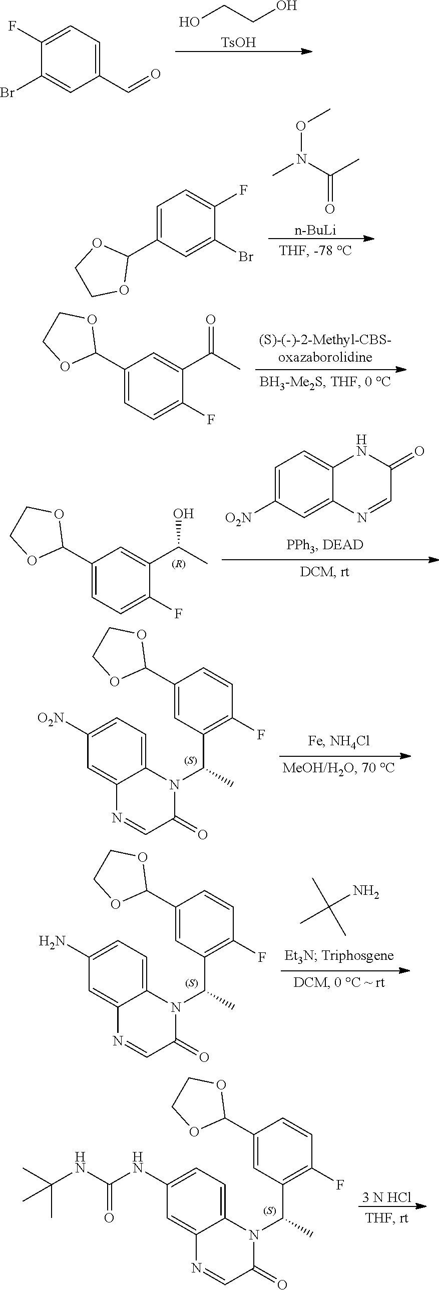

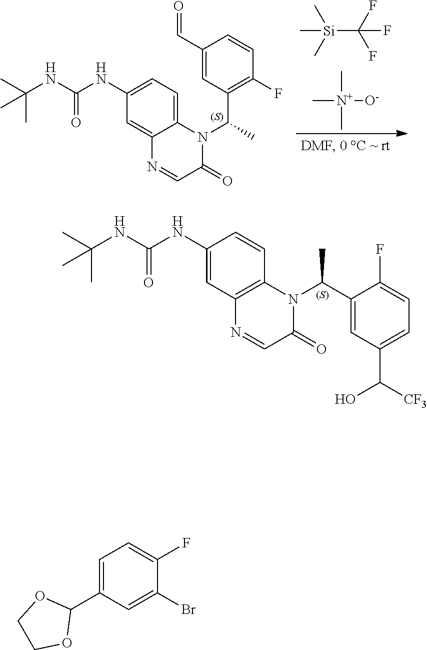

### 2-(3-bromo-4-fluorophenyl)-1,3-dioxolane

To a solution of 3-bromo-4-fluorobenzaldehyde (10 g, 49.259 mmol, 1 equiv) in Toluene (50 mL) was added ethylene glycol (9477.90 mg, 152.703 mmol, 3.1 equiv) and para-toluene sulfonate (1696.47 mg, 9.852 mmol, 0.2 equiv). The resulting mixture was stirred for 3 h at 120° C. The mixture was diluted with EtOAc (100 mL), washed with water (3×50 mL), brine (2×50 mL) and concentrated under vacuum to yield a crude product which was directly purified by silica gel column, eluted with PE/EA (3:1) to afford 2-(3-bromo-4-fluorophenyl)-1,3-dioxolane (10 g, 82.17%) as a white solid. MS (ESI): mass calcd. for C₉H₈BrFO₂: 245.97 m/z, found 247.00 [M+H, M+H+2]⁺.

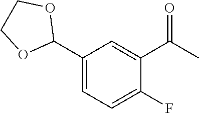

### 1-[5-(1,3-dioxolan-2-yl)-2-fluorophenyl]ethanone

To a solution of 2-(3-bromo-4-fluorophenyl)-1,3-dioxolane (2 g, 8.095 mmol, 1 equiv) in THF (20 mL) was added butyllithium (601.54 mg, 9.390 mmol, 1.16 equiv) dropwise at −78° C. under nitrogen atmosphere. The resulting mixture was stirred for 1 h at −78° C. N-methoxy-N-methylacetamide (868.17 mg, 8.419 mmol, 1.04 equiv) was added to the reaction at −78° C. under nitrogen atmosphere. The resulting mixture was stirred for 1 h at rt. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 1-[5-(1,3-dioxolan-2-yl)-2-fluorophenyl]ethanone (1.2 g, 70.52%) as a yellow solid. MS (ESI): mass calcd. for C₁₁H₁₁FO₃: 210.07 m/z, found 211.00 [M+H]⁺.

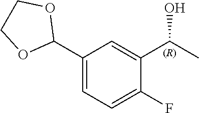

### (1R)-1-[5-(1,3-dioxolan-2-yl)-2-fluorophenyl]ethanol

To a solution of CBS (1001.50 mg, 3.806 mmol, 0.4 equiv) in THF (20 mL) was added BH₃-Me₂S (1084.09 mg, 14.273 mmol, 1.5 equiv) and 1-[5-(1,3-dioxolan-2-yl)-2-fluorophenyl]ethanone (2 g, 9.515 mmol, 1 equiv) dropwise at 0° C. The resulting mixture was stirred for 1 h at 0° C. The mixture was diluted with EtOAc (100 mL), washed with water (3×50 mL), brine (2×50 mL) and concentrated under vacuum to yield a crude product which was directly purified by silica gel column, eluted with PE/EA (3:1) to afford (1R)-1-[5-(1,3-dioxolan-2-yl)-2-fluorophenyl]ethanol (1.5 g, 74.29%) as a white solid. MS (ESI): mass calcd. for C₁₁H₁₃FO₃: 212.08 m/z, found 213.30 [M+H]⁺.

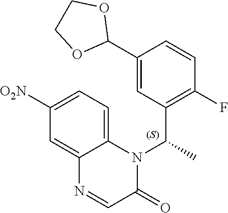

### 1-[(1S)-1-[5-(1,3-dioxolan-2-yl)-2-fluorophenyl]ethyl]-6-nitroquinoxalin-2-one

To a solution of (1R)-1-[5-(1,3-dioxolan-2-yl)-2-fluorophenyl]ethanol (1.6 g, 7.539 mmol, 1 equiv) in DCM (20 mL) was added 6-nitro-1H-quinoxalin-2-one (1.44 g, 7.539 mmol, 1 equiv) and PPh₃ (2.97 g, 11.308 mmol, 1.5 equiv) dropwise under nitrogen atmosphere. The resulting mixture was stirred for 0.5 h at rt. DEAD (1.97 g, 11.308 mmol, 1.5 equiv) was added to the reaction under nitrogen atmosphere. The resulting mixture was stirred for 1 h at rt. The reaction mixture was quenched by water and extracted with EA (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 1-[(1S)-1-[5-(1,3-dioxolan-2-yl)-2-fluorophenyl]ethyl]-6-nitroquinoxalin-2-one (1.2 g, 41.30%) as a yellow solid. MS (ESI): mass calcd. for C₁₉H₁₆FN₃O₅: 385.10 m/z, found 386.00 [M+H]⁺.

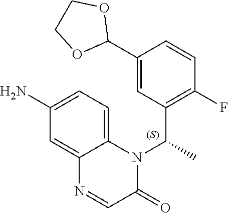

### 6-amino-1-[(1S)-1-[5-(1,3-dioxolan-2-yl)-2-fluorophenyl]ethyl]quinoxalin-2-one

To a solution of 1-[(1S)-1-[5-(1,3-dioxolan-2-yl)-2-fluorophenyl]ethyl]-6-nitroquinoxalin-2-one (1.2 g, 3.114 mmol, 1 equiv) in MeOH (15 mL) and H₂O (1.5 mL) was added Fe (1.74 g, 31.140 mmol, 10 equiv) and NH₄C₁ (1.67 g, 31.140 mmol, 10 equiv). The resulting mixture was stirred for 3 h at 70° C. After cooling down to rt, the mixture was diluted with EtOAc (100 mL), washed with water (3×50 mL), brine (2×50 mL) and concentrated under vacuum to yield a crude product which was directly purified by silica gel column, eluted with PE/EA (3:1) to afford 6-amino-1-[(1S)-1-[5-(1,3-dioxolan-2-yl)-2-fluorophenyl]ethyl]quinoxalin-2-one (200 mg, 18.07%) as a white solid. MS (ESI): mass calcd. For C₁₉H₁₈FN₃O₃: 355.13 m/z, found 356.20 [M+H]⁺.

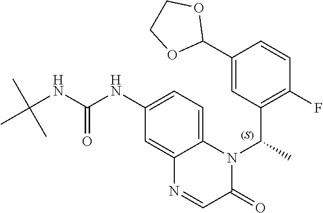

### 3-tert-butyl-1-{1-[(1S)-1-[5-(1,3-dioxolan-2-yl)-2-fluorophenyl]ethyl]-2-oxoquinoxalin-6-yl}urea

To a solution of 6-amino-1-[(1S)-1-[5-(1,3-dioxolan-2-yl)-2-fluorophenyl]ethyl]quinoxalin-2-one (200 mg, 0.563 mmol, 1 equiv) in DCM (10 mL) was added triphosgene (167.01 mg, 0.563 mmol, 1 equiv) dropwise at 0° C. under nitrogen atmosphere. The resulting mixture was stirred for 10 min at rt. TEA (113.90 mg, 1.126 mmol, 2 equiv) was added to the reaction under nitrogen atmosphere. The resulting mixture was stirred for 15 min at rt. Then erbumine (82.32 mg, 1.126 mmol, 2 equiv) and TEA (113.90 mg, 1.126 mmol, 2 equiv) was added to the reaction under nitrogen atmosphere. The resulting mixture was stirred for 1 h at rt. The reaction mixture was quenched by water and extracted with DCM (3×30 mL). The combined organic extracts were washed with brine (20 mL), dried over anhydrous Na₂SO₄, and concentrated under vacuum to yield a crude product which was directly purified by silica gel column, eluted with PE/EA (2:1) to afford 3-tert-butyl-1-{1-[(1S)-1-[5-(1,3-dioxolan-2-yl)-2-fluorophenyl]ethyl]-2-oxoquinoxalin-6-yl}urea (150 mg, 58.64%) as a yellow solid. MS (ESI): mass calcd. for C₂₄H₂₇FN₄O₄: 454.20 m/z, found 455.30 [M+H]⁺.

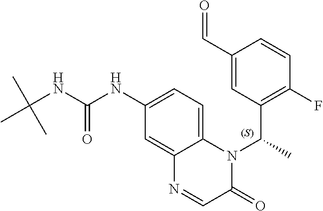

### 3-tert-butyl-1-{1-[(1S)-1-(2-fluoro-5-formylphenyl)ethyl]-2-oxoquinoxalin-6-yl}urea

To a solution of 3-tert-butyl-1-{1-[(1S)-1-[5-(1,3-dioxolan-2-yl)-2-fluorophenyl]ethyl]-2-oxoquinoxalin-6-yl}urea (130 mg, 0.286 mmol, 1 equiv) in THF (7 mL) was added 3N HCl (3.5 mL) dropwise at 0° C. The resulting mixture was stirred for 1 h at rt. The mixture was diluted with EtOAc (20 mL), washed with water (3×10 mL), brine (2×10 mL) and concentrated under vacuum to afford 3-tert-butyl-1-{1-[(1S)-1-(2-fluoro-5-formylphenyl)ethyl]-2-oxoquinoxalin-6-yl}urea (85 mg, 72.4%) as a white solid. MS (ESI): mass calcd. for C₂₂H₂₃FN₄O₃: 410.17 m/z, found 411.30 [M+H]⁺.

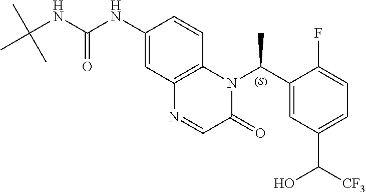

### 3-tert-butyl-1-{1-[(1S)-1-[2-fluoro-5-(2,2,2-trifluoro-1-hydroxyethyl)phenyl]ethyl]-2-oxoquinoxalin-6-yl}urea

To a solution of 3-tert-butyl-1-{1-[(1S)-1-(2-fluoro-5-formylphenyl)ethyl]-2-oxoquinoxalin-6-yl}urea (150 mg, 0.365 mmol, 1 equiv) in dimethylformamide (10 mL) was added trimethyl(trifluoromethyl)silane (207.86 mg, 1.460 mmol, 4 equiv) and N,N-dimethylmethanamine oxide (109.80 mg, 1.460 mmol, 4 equiv) dropwise at 0° C. The resulting mixture was stirred for 8 h at rt. The reaction mixture was quenched by water and extracted with EA (3×20 mL). The combined organic extracts were washed with brine (20 mL), dried over anhydrous Na₂SO₄, and concentrated under vacuum to yield a crude product which was directly purified by silica gel column eluted with PE/EA (2:1) & Prep-HPLC (Column: YMC Triart C18 ExRs Column, 20*250 mm, 5 μm; Mobile Phase A: Water (10 mmol/L NH₄HCO₃), Mobile Phase B: ACN; Flow rate: 25 mL/min mL/min; Gradient: 41% B to 71% B in 7 min; Wave Length: 254 nm/220 nm nm; RT1(min): 6.42 Column: YMC Triart C18 ExRs Column, 20*250 mm, 5 μm; Mobile Phase A: Water (10 mmol/L NH₄HCO₃), Mobile Phase B: ACN; Flow rate: 25 mL/min mL/min; Gradient: 41% B to 71% B in 7 min; Wave Length: 254 nm/220 nm nm; RT1(min): 6.42) to afford 3-tert-butyl-1-{1-[(1S)-1-[2-fluoro-5-(2,2,2-trifluoro-1-hydroxyethyl)phenyl]ethyl]-2-oxoquinoxalin-6-yl}urea (2.8 mg, 1.57%) as an light yellow. MS (ESI): mass calcd. For C₂₃H₂₄F₄N₄O₃, 480.18 m/z, found 481.25 [M+H]⁺. ¹H NMR (300 MHz, DMSO-d₆) δ 8.45 (s, 1H), 8.17 (s, 1H), 7.79-8.00 (m, 2H), 7.29-7.55 (m, 3H), 7.13 (dd, J=10.8, 8.5 Hz, 1H), 6.97 (s, 1H), 6.57 (s, 1H), 6.01 (s, 1H), 5.28 (d, J=7.3 Hz, 1H), 1.88 (d, J=7.0 Hz, 3H), 1.29 (s, 9H). ¹⁹F NMR (282 MHz, DMSO) δ −76.88, −116.10.

### Example 376: 1-(tert-butyl)-3-((R)-3-methyl-2-oxo-1-((S)-1-phenylethyl)-7-(trifluoromethyl)-2,3-dihydro-1H-pyrido[2,3-b][1,4]oxazin-6-yl)urea

### Synthetic Scheme

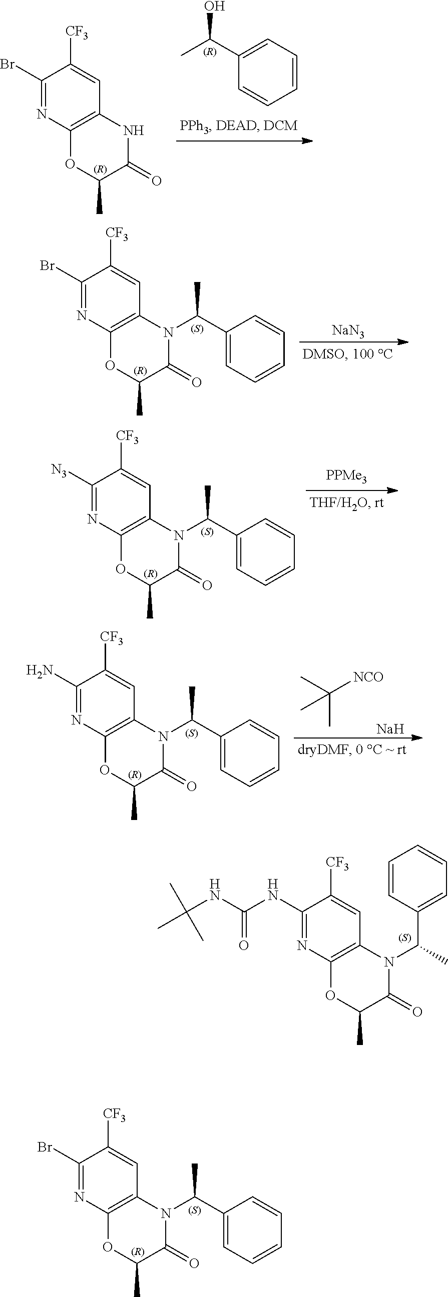

### (R)-6-bromo-3-methyl-1-((S)-1-phenylethyl)-7-(trifluoromethyl)-1H-pyrido[2,3-b][1,4]oxazin-2(3H)-one

A solution of (3R)-6-bromo-3-methyl-7-(trifluoromethyl)-1H,3H-pyrido[2,3-b][1,4]oxazin-2-one (50 mg, 0.161 mmol, 1 equiv) in DCM (5 mL) was treated with (R)-1-phenylethan-1-ol (29.46 mg, 0.241 mmol, 1.5 equiv) and PPh₃ (63.24 mg, 0.241 mmol, 1.5 equiv) for 1 h at rt under nitrogen atmosphere followed by the addition of DEAD (41.99 mg, 0.241 mmol, 1.5 equiv) dropwise at 0° C. The resulting mixture was stirred for 2 h at rt under N₂ atmosphere. Quenched with water (10 mL) and extracted with DCM (3×10 mL). The combined extracts were washed with water, brine, dried over Na₂SO₄, filtered and concentrated. The residue was purified by column chromatography on silica gel with EA/PE (0-100%) to give (3R)-6-bromo-3-methyl-1-[(1S)-1-phenylethyl]-7-(trifluoromethyl)-3H-pyrido[2,3-b][1,4]oxazin-2-one (40 mg, 59.93%) as a yellow solid. LC/MS: mass calcd. for C₁₇H₁₄BrF₃N₂O₂: 414.02 m/z, found: 415.10, 417.10 [M+H, M+H+2]⁺.

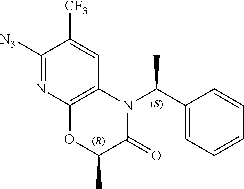

### (R)-6-azido-3-methyl-1-((S)-1-phenylethyl)-7-(trifluoromethyl)-1H-pyrido[2,3-b][1,4]oxazin-2(3H)-one

To a solution of (3R)-6-bromo-3-methyl-1-[(1S)-1-phenylethyl]-7-(trifluoromethyl)-3H-pyrido[2,3-b][1,4]oxazin-2-one (100 mg, 0.241 mmol, 1 equiv) in DMSO (3 mL) was added NaN₃ (31.31 mg, 0.482 mmol, 2 equiv). The reaction was stirred at 100° C. under N₂ for 0.5 h. After cooling down to rt, the reaction was quenched with water (10 mL) and extracted with EA (3×10 mL). The mixture was allowed to cool down to rt. The combined extracts were washed with water, brine, dried over Na₂SO₄, filtered and concentrated. The residue was purified by column chromatography on silica gel with EA/PE (0-100%) to give (3R)-6-azido-3-methyl-1-[(1S)-1-phenylethyl]-7-(trifluoromethyl)-3H-pyrido[2,3-b][1,4]oxazin-2-one (31 mg, 30.70%) as a white solid. MS (ESI): mass calcd. for C₁₇H₁₄F₃N₅O₂: 377.11 m/z, found: 378.05 [M+H]⁺.

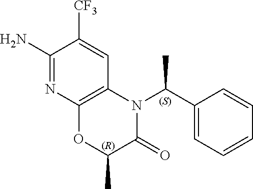

### (R)-6-amino-3-methyl-1-((S)-1-phenylethyl)-7-(trifluoromethyl)-1H-pyrido[2,3-b][1,4]oxazin-2(3H)-one

To a solution of 6-azido-1-[(1S)-1-phenylethyl]-7-(trifluoromethyl)-3H-pyrido[2,3-b][1,4]oxazin-2-one (60 mg, 0.165 mmol, 1 equiv) in THF (2.5 mL)/H₂O (0.5 mL) was added trimethylphosphine (125.65 mg, 1.650 mmol, 10 equiv). The reaction was stirred at rt for 1 h. Quenched with water (5 mL) and extracted with EA (3×5 mL). The combined extracts were washed with water, brine, dried over Na₂SO₄, filtered and concentrated. The residue was purified by column chromatography on silica gel with EA/PE (0-100%) to give 6-amino-1-[(1S)-1-phenylethyl]-7-(trifluoromethyl)-3H-pyrido[2,3-b][1,4]oxazin-2-one (50 mg, 80.78%) as a white solid. MS (ESI): mass calcd. for C₁₇H₁₆F₃N₃O₂: 351.12 m/z, found: 352.05 [M+H]⁺.

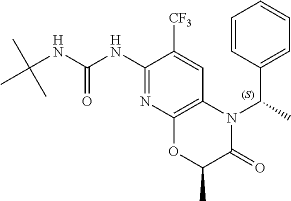

### 1-(tert-butyl)-3-((R)-3-methyl-2-oxo-1-((S)-1-phenylethyl)-7-(trifluoromethyl)-2,3-dihydro-1H-pyrido[2,3-b][1,4]oxazin-6-yl)urea

To a solution of (3R)-6-amino-3-methyl-1-[(1S)-1-phenylethyl]-7-(trifluoromethyl)-3H-pyrido[2,3-b][1,4]oxazin-2-one (100 mg, 0.285 mmol, 1 equiv) in dry-DMF (3 mL) was added NaH (6.83 mg, 0.285 mmol, 1 equiv). The reaction was stirred at rt for 0.5 h. The reaction was added 2-isocyanato-2-methylpropane (84.65 mg, 0.855 mmol, 3 equiv) and stirred at rt for 1 h. Quenched with water (10 mL) and extracted with EA (3×10 mL). The combined extracts were washed with water, brine, dried over Na₂SO₄, filtered and concentrated. The residue was purified by preparative HPLC using a YMC-Actus Triart C18 ExRS 150 mm×30 mm×5 μm column (eluent: 49% to 73% (v/v) CH₃CN and H₂O with 10 mmol/L NH₄HCO₃) to afford the title compound 3-tert-butyl-1-[(3R)-3-methyl-2-oxo-1-[(1S)-1-phenylethyl]-7-(trifluoromethyl)-3H-pyrido[2,3-b][1,4]oxazin-6-yl]urea (38.2 mg, 29.42%) as a white solid. LC/MS (ESI): mass calcd. for C₂₂H₂₅F₃N₄O₃: 450.19 m/z, found: 451.20 [M+H]⁺. ¹H NMR (300 MHz, DMSO-d₆) δ 7.69 (s, 1H), 7.19-7.47 (m, 7H), 6.07-6.26 (m, 1H), 5.13-5.24 (m, 1H), 1.74-1.76 (m, 3H), 1.55-1.57 (m, 3H), 1.28 (s, 9H). ¹⁹F NMR (282 MHz, DMSO) δ −59.29, −59.33, −59.41.

### Example 377: (S)-1-(tert-butyl)-3-(6-oxo-5-(1-phenylethyl)-3-(trifluoromethyl)-5,6,7,8-tetrahydro-1,5-naphthyridin-2-yl)urea

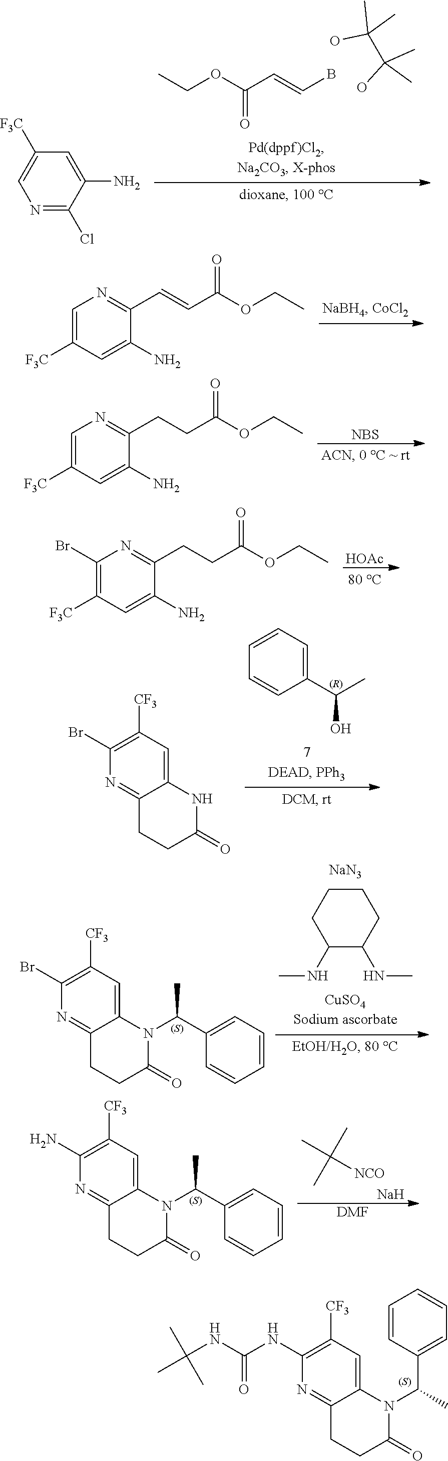

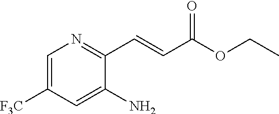 [SMILES: CCOC(=O)/C=C/c1ncc(C(F)(F)F)cc1N]

### ethyl (E)-3-(3-amino-5-(trifluoromethyl)pyridin-2-yl)acrylate

To a stirred mixture of 2-chloro-5-(trifluoromethyl) pyridin-3-amine (3 g, 15.263 mmol, 1 equiv) in dioxane (40 mL) and H₂O (10 mL) was added ethyl (E)-3-(4,4,5,5-tetramethyl-1,3,2-dioxaborolan-2-yl)acrylate (5.18 g, 22.895 mmol, 1.5 equiv), Pd(dppf)Cl₂ (1.12 g, 1.526 mmol, 0.1 equiv), Na₂CO₃ (4.85 g, 45.789 mmol, 3 equiv) and X-phos (727.61 mg, 1.526 mmol, 0.1 equiv) at room temperature under air atmosphere. The resulting mixture was maintained under nitrogen and stirred at 90° C. for 3 h. After cooling down to room temperature, the reaction was quenched with water (60 mL). The resulting mixture was extracted with ethyl acetate (3×60 mL). The organic layers were combined, dried over anhydrous Na₂SO₄, filtered and concentrated. The residue obtained was purified by silica gel chromatography (0-50% ethyl acetate/petroleum ether) to afford the ethyl (E)-3-(3-amino-5-(trifluoromethyl) pyridin-2-yl)acrylate (2.5 g, 62.95%) as a yellow oil. LC/MS: mass calcd. for C₁₁H₁₁F₃N₂O₂: 260.08, found: 261.15 [M+H]⁺.

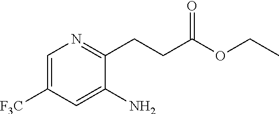 [SMILES: CCOC(=O)CCc1ncc(C(F)(F)F)cc1N]

### ethyl 3-(3-amino-5-(trifluoromethyl)pyridin-2-yl)propanoate

To a stirred mixture of ethyl (E)-3-(3-amino-5-(trifluoromethyl) pyridin-2-yl)acrylate (1 g, 3.843 mmol, 1 equiv) in THF (15 mL) was added NaBH₄ (726.90 mg, 19.215 mmol, 5 equiv) and CoCl₂ (249.47 mg, 1.921 mmol, 0.5 equiv) at 0 degrees C. under air atmosphere. The resulting mixture was maintained under air and stirred at 25° C. for 3 h. The reaction was quenched with water (30 mL). The resulting mixture was extracted with ethyl acetate (3×30 mL). The organic layers were combined, dried over anhydrous Na₂SO₄, filtered and concentrated. The residue obtained was purified by silica gel chromatography (0-50% ethyl acetate/petroleum ether) to afford the ethyl 3-(3-amino-5-(trifluoromethyl) pyridin-2-yl)propanoate (750 mg, 74.42%) as a yellow oil. LC/MS: mass calcd. for C₁₁H₁₃F₃N₂O₂: 262.09, found: 263.15 [M+H]⁺.

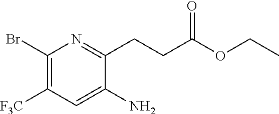 [SMILES: CCOC(=O)CCc1nc(Br)c(C(F)(F)F)cc1N]

### ethyl 3-(3-amino-6-bromo-5-(trifluoromethyl)pyridin-2-yl)propanoate

To a stirred mixture of ethyl 3-(3-amino-5-(trifluoromethyl)pyridin-2-yl)propanoate (700 mg, 2.669 mmol, 1 equiv) in ACN (5 mL) was added NBS (570.13 mg, 3.203 mmol, 1.2 equiv) at 0 degrees C. under air atmosphere. The resulting mixture was maintained under air and stirred at 0° C. for 3 h. The reaction was quenched with water (20 mL). The resulting mixture was extracted with ethyl acetate (3×20 mL). The organic layers were combined, dried over anhydrous Na₂SO₄, filtered and concentrated. The residue obtained was purified by silica gel chromatography (0-50% ethyl acetate/petroleum ether) to afford the ethyl 3-(3-amino-6-bromo-5-(trifluoromethyl)pyridin-2-yl)propanoate (350 mg, 38.44%) as a yellow oil. LC/MS: mass calcd. for C₁₁H₁₂BrF₃N₂O₂: 340.00, found: 341.05, 343.05 [M+H, M+H+2]⁺.

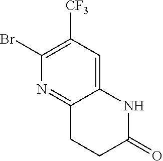 [SMILES: O=C1CCc2nc(Br)c(C(F)(F)F)cc2N1]

### 6-bromo-7-(trifluoromethyl)-3,4-dihydro-1,5-naphthyridin-2(1H)-one

The ethyl 3-(3-amino-6-bromo-5-(trifluoromethyl)pyridin-2-yl)propanoate (300 mg, 0.879 mmol, 1 equiv) was dissolved in HOAc (3 mL) at 0 degrees C. under air atmosphere. The resulting mixture was maintained under air and stirred at 80° C. for 3 h. After cooling down to room temperature, the reaction was quenched with NaHCO₃ solution (20 mL). The resulting mixture was extracted with ethyl acetate (3×10 mL). The organic layers were combined, dried over anhydrous Na₂SO₄, filtered and concentrated. The residue obtained was purified by silica gel chromatography (0-60% ethyl acetate/petroleum ether) to afford the 6-bromo-7-(trifluoromethyl)-3,4-dihydro-1,5-naphthyridin-2(1H)-one (250 mg, 96.34%) as a yellow oil. LC/MS: mass calcd. for C₉H₆BrF₃N₂O: 293.96, found: 295.05, 297.05 [M+H, M+H+2]⁺.

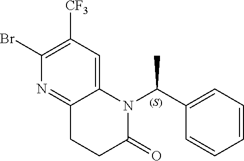

### (S)-6-bromo-1-(1-phenylethyl)-7-(trifluoromethyl)-3,4-dihydro-1,5-naphthyridin-2(1H)-one

To a stirred mixture of 6-bromo-7-(trifluoromethyl)-3,4-dihydro-1,5-naphthyridin-2(1H)-one (240 mg, 0.813 mmol, 1 equiv) in DCM (5 mL) was added (R)-1-phenylethan-1-ol (149.06 mg, 1.220 mmol, 1.5 equiv) and PPh₃ (320.02 mg, 1.220 mmol, 1.5 equiv) at 0 degrees C. under air atmosphere. After stirring at 25° C. for 30 min under nitrogen, DEAD (212.49 mg, 1.220 mmol, 1.5 equiv) was added to the reaction system at 0 degrees C. The resulting mixture was maintained under nitrogen and stirred at 25° C. for 3 h. The reaction was quenched with water (10 mL). The resulting mixture was extracted with ethyl acetate (3×10 mL). The organic layers were combined, dried over anhydrous Na₂SO₄, filtered and concentrated. The residue obtained was purified by silica gel chromatography (0-100% ethyl acetate/petroleum ether) to afford the (S)-6-bromo-1-(1-phenylethyl)-7-(trifluoromethyl)-3,4-dihydro-1,5-naphthyridin-2(1H)-one (140 mg, 43.11%) as a yellow oil. LC/MS: mass calcd. for C₁₇H₁₄BrF₃N₂O: 398.02, found: 399.10, 401.10 [M+H, M+H+2]⁺.

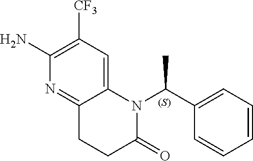

### (S)-6-amino-1-(1-phenylethyl)-7-(trifluoromethyl)-3,4-dihydro-1,5-naphthyridin-2(1H)-one

To a stirred mixture of (S)-6-bromo-1-(1-phenylethyl)-7-(trifluoromethyl)-3,4-dihydro-1,5-naphthyridin-2(1H)-one (130 mg, 0.326 mmol, 1 equiv) in EtOH (2 mL) and H₂O (1 mL) was added NaN₃ (63.51 mg, 0.978 mmol, 3 equiv), N₁,N₂-dimethylcyclohexane-1,2-diamine (27.79 mg, 0.196 mmol, 0.6 equiv), CuSO₄ (51.97 mg, 0.326 mmol, 1 equiv) and sodium ascorbate (129.68 mg, 0.652 mmol, 2 equiv) at room temperature under air atmosphere. The resulting mixture was maintained under nitrogen and stirred at 80° C. for 3 h. After cooling down to room temperature, the reaction was quenched with water (10 mL). The resulting mixture was extracted with ethyl acetate (3×10 mL). The organic layers were combined, dried over anhydrous Na₂SO₄, filtered and concentrated. The residue obtained was purified by silica gel chromatography (0-100% ethyl acetate/petroleum ether) to afford the (S)-6-amino-1-(1-phenylethyl)-7-(trifluoromethyl)-3,4-dihydro-1,5-naphthyridin-2(1H)-one (50 mg, 45.79%) as a yellow oil. LC/MS: mass calcd. for C₁₇H₁₆F₃N₃O: 335.12, found: 336.00 [M+H]⁺.

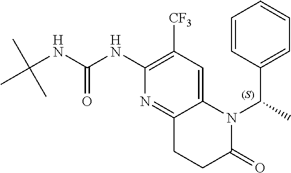

### (R)-6-amino-1-benzyl-3-methyl-7-(trifluoromethyl)-1H-pyrido[2,3-b][1,4]oxazin-2(3H)-one

To a stirred mixture of (S)-6-amino-1-(1-phenylethyl)-7-(trifluoromethyl)-3,4-dihydro-1,5-naphthyridin-2(1H)-one (45 mg, 0.134 mmol, 1 equiv) in DMF (2 mL) was added NaH (4.83 mg, 0.201 mmol, 1.5 equiv) at 0 degrees C. under air atmosphere. After stirring at 0 degrees C. for 30 min, 2-isocyanato-2-methylpropane (39.91 mg, 0.402 mmol, 3 equiv) was added to the reaction system. The resulting mixture was stirred at 25° C. for 3 h. Quenched with water (5 mL) and extracted with EA (3×5 mL). The combined extracts were washed with water, brine, dried over Na₂SO₄, filtered and concentrated. The residue was purified by preparative HPLC using a XBridge Shield RP18 OBD Column 150 mm×30 mm×5 μm column (eluent: 70% to 85% (v/v) Me and Water with 0.1% FA) to afford the title compound (S)-1-(tert-butyl)-3-(6-oxo-5-(1-phenylethyl)-3-(trifluoromethyl)-5,6,7,8-tetrahydro-1,5-naphthyridin-2-yl)urea (12.9 mg, 21.43%) as a white solid. LC/MS (ESI): mass calcd. for C₂₂H₂₅F₃N₄O₂:434.19 m/z, found: 435.25 [M+H]⁺. ¹H-NMR (300 MHz, DMSO-d₆) δ 8.00 (s, 1H), 7.51 (s, 1H), 7.23-7.44 (m, 5H), 7.06 (s, 1H), 6.16-6.32 (m, 1H), 2.95-3.15 (m, 2H), 2.87-2.96 (m, 1H), 2.76-2.82 (m, 1H), 1.68 (d, J=7.2 Hz, 3H), 1.31 (s, 9H). ¹⁹F-NMR (282 MHz, DMSO) δ −60.51.

### Example 378: 1-[(3S)-1-benzyl-3-methyl-2-oxo-7-(trifluoromethyl)-3H-pyrido[2,3-b][1,4]oxazin-6-yl]-3-tert-butylurea

### Synthetic Scheme

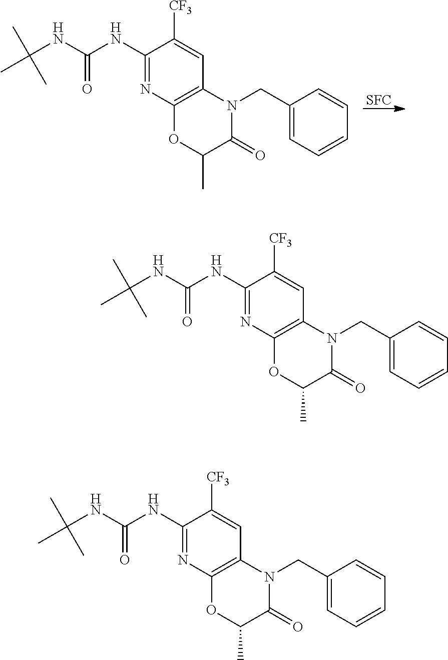

### 1-[(3S)-1-benzyl-3-methyl-2-oxo-7-(trifluoromethyl)-3H-pyrido[2,3-b][1,4]oxazin-6-yl]-3-tert-butylurea

The crude product (130 mg) was separated by chiral-HPLC using a CHIRALPAK IA, 5*25 cm, 5 μm (eluent: 35% to 35% (v/v): EtOH—HPLC and Hex(0.5% 2M NH₃-MeOH) to yield second enantiomer example 1-[(3S)-1-benzyl-3-methyl-2-oxo-7-(trifluoromethyl)-3H-pyrido[2,3-b][1,4]oxazin-6-yl]-3-tert-butylurea (20 mg, 15.09%) as a white solid. LC/MS: mass calcd. for C₂₁H₂₃F₃N₄O₃: 436.2, found: 437.20 [M+H]⁺. ¹H NMR (400 MHz, DMSO-d₆) δ 7.72 (s, 1H), 7.58 (s, 1H), 7.30-7.40 (m, 2H), 7.21-7.30 (m, 4H), 5.13-5.34 (m, 3H), 1.60 (d, J=6.8 Hz, 3H), 1.29 (s, 6H). ¹⁹F NMR (376 MHz, DMSO) δ −58.74.

hTSHR TR-FRET cAMP Assay

Assays were performed using Expi293F Inducible cells (Invitrogen) stably expressing hTSHR via a pcZeo TetO DNA plasmid. Cell lines were maintained in suspension in Expi293 Expression Medium (ThermoFisher Scientific) supplemented with 10 g/mL Blasticidin and 10 μg/mL Zeocin and incubated at 37° C., 8% CO₂, with shaking. hTSHR cells were incubated overnight at 32° C., 5% C₀₂, with shaking, but without Doxycycline. Assay-ready aliquots were prepared by harvesting each cell type separately 24-hours post-induction. Cells were pelleted at 4° C., resuspended in Expi293 Expression media+10% DMSO, aliquoted, and kept frozen at −80° C. until ready for use.

For the assay, concentration-response curves of test and reference compounds were added to 384-well plates using an Echo 650 liquid handler (Beckman Coulter) and backfilled with DMSO to a final concentration of 0.5%. cAMP was measured using the cisbio cAMP Gs dynamic HTRF kit (PerkinElmer) according to manufacturer instructions. Aliquots of frozen hTSHR cells were quickly thawed and washed with phosphate-buffered saline (Sigma-Aldrich) to remove media and DMSO. The cells were resuspended in kit-supplied Stimulation Buffer at 0.8×10⁶ cells/mL. 7.5 μL of the hTSHR cell dilution were added to each well of the assay plate and incubated with the compounds for 15 minutes in a 37 C, 0% CO₂ incubator. 2.5 μL of Vehicle control or an EC₈₀ concentration of recombinant human anti-TSHR antibody (M22, Creative Biolabs) was added to stimulate cAMP production. Cells were incubated with for 30 minutes at 37° C., 0% CO₂. Following this incubation, the cells were lysed and accumulated cAMP was detected through the addition of kit-supplied lysis buffer containing d2-reagent and Eu-cryptate antibody. The HTRF signal was quantified using a BMG Clariostar plate reader optimized for HTRF assays. The HTRF ratio was determined by dividing the signal output at 655 nm by that at 620 nm. Data were normalized to the signal produced by the EC₈₀ concentration of either M22 or FSH (0% inhibition) and vehicle (no agonist, 100% inhibition). Results of the assay are provided in Table 17.

TABLE 17

| Example | IUPAC Name | Activity* |
|---|---|---|
| 1 | 1-(1-benzyl-2-oxo-1,2,3,4-tetrahydroquinolin-6-yl)-3-(tert- | +++ |
| | butyl)urea | |
| 2 | 1-(tert-butyl)-3-(2-oxo-1-(pyridin-2-ylmethyl)-1,2,3,4- | + |
| | tetrahydroquinolin-6-yl)urea | |
| 3 | 1-(tert-butyl)-3-(2-oxo-1-(1-phenylethyl)-1,2,3,4- | +++ |
| | tetrahydroquinolin-6-yl)urea | |
| 4 | 1-(tert-butyl)-3-(1-((1-methyl-1H-pyrazol-3-yl)methyl)-2-oxo- | + |
| | 1,2,3,4-tetrahydroquinolin-6-yl)urea | |
| 5 | 1-(1-benzyl-3-methyl-2-oxo-1,2,3,4-tetrahydroquinolin-6-yl)-3- | +++ |
| | (tert-butyl)urea | |
| 6 | 1-(tert-butyl)-3-(1-(3-chlorobenzyl)-2-oxo-1,2,3,4- | +++ |
| | tetrahydroquinolin-6-yl)urea | |
| 7 | 1-(4-benzyl-3-oxo-3,4-dihydro-2H-benzo[b][1,4]oxazin-7-yl)-3- | +++ |
| | (tert-butyl)urea | |
| 8 | 1-(tert-butyl)-3-(1-(2-chlorobenzyl)-2-oxo-1,2,3,4- | ++ |
| | tetrahydroquinolin-6-yl)urea | |
| 9 | tert-butyl (1-benzyl-2-oxo-1,2,3,4-tetrahydroquinolin-6- | +++ |
| | yl)carbamate | |
| 10 | 1-(1-benzyl-4,4-dimethyl-2-oxo-1,2,3,4-tetrahydroquinolin-6-yl)-3- | + |
| | (tert-butyl)urea | |
| 11 | 1-(1-benzyl-3-(methoxymethyl)-2-oxo-1,2,3,4-tetrahydroquinolin- | ++ |
| | 6-yl)-3-(tert-butyl)urea | |
| 12 | 1-(1-benzyl-3-methyl-2-oxo-1,2,3,4-tetrahydroquinazolin-6-yl)-3- | ++ |
| | (tert-butyl)urea | |
| 13 | (S)-1-(tert-butyl)-3-(2-oxo-1-(1-phenylethyl)-1,2,3,4- | +++ |
| | tetrahydroquinolin-6-yl)urea | |
| 14 | (R)-1-(tert-butyl)-3-(2-oxo-1-(1-phenylethyl)-1,2,3,4- | + |
| | tetrahydroquinolin-6-yl)urea | |
| 15 | 1-(1-benzyl-4-methyl-2-oxo-1,2,3,4-tetrahydroquinolin-6-yl)-3- | + |
| | (tert-butyl)urea | |
| 16 | 1-(1-benzyl-2-oxo-1,2,3,4-tetrahydroquinolin-6-yl)-3-(4-hydroxy-2- | ++ |
| | methylbutan-2-yl)urea | |
| 17 | 1-(tert-butyl)-3-(1-(4-cyanobenzyl)-2-oxo-1,2,3,4- | + |
| | tetrahydroquinolin-6-yl)urea | |
| 18 | 1-(tert-butyl)-3-(1-(3-cyanobenzyl)-2-oxo-1,2,3,4- | + |
| | tetrahydroquinolin-6-yl)urea | |
| 19 | 1-(tert-butyl)-3-(1-(2-fluorobenzyl)-2-oxo-1,2,3,4- | +++ |
| | tetrahydroquinolin-6-yl)urea | |
| 20 | 1-(tert-butyl)-3-(1-(2-cyanobenzyl)-2-oxo-1,2,3,4- | +++ |
| | tetrahydroquinolin-6-yl)urea | |
| 21 | 1-(tert-butyl)-3-(1-(3-methylbenzyl)-2-oxo-1,2,3,4- | +++ |
| | tetrahydroquinolin-6-yl)urea | |
| 22 | 1-(tert-butyl)-3-(1-(3-methoxybenzyl)-2-oxo-1,2,3,4- | +++ |
| | tetrahydroquinolin-6-yl)urea | |
| 23 | 1-(tert-butyl)-3-(2-oxo-1-(3-(trifluoromethyl)benzyl)-1,2,3,4- | +++ |
| | tetrahydroquinolin-6-yl)urea | |
| 24 | 1-(tert-butyl)-3-(1-(2,5-dichlorobenzyl)-2-oxo-1,2,3,4- | +++ |
| | tetrahydroquinolin-6-yl)urea | |
| 25 | 1-(tert-butyl)-3-(1-(3,5-dichlorobenzyl)-2-oxo-1,2,3,4- | +++ |
| | tetrahydroquinolin-6-yl)urea | |
| 26 | 1-(tert-butyl)-3-(1-(3-fluorobenzyl)-2-oxo-1,2,3,4- | +++ |
| | tetrahydroquinolin-6-yl)urea | |
| 27 | 1-(tert-butyl)-3-(1-(4-fluorobenzyl)-2-oxo-1,2,3,4- | ++ |
| | tetrahydroquinolin-6-yl)urea | |
| 28 | 1-(tert-butyl)-3-(2-oxo-1-(1-phenylpropyl)-1,2,3,4- | + |
| | tetrahydroquinolin-6-yl)urea | |
| 29 | 1-(1-benzyl-3,3-dimethyl-2-oxo-1,2,3,4-tetrahydroquinolin-6-yl)-3- | + |
| | (tert-butyl)urea | |
| 30 | 1-(tert-butyl)-3-(1-(2-hydroxy-1-phenylethyl)-2-oxo-1,2,3,4- | + |
| | tetrahydroquinolin-6-yl)urea | |
| 31 | 1-(1-benzyl-2-oxoindolin-5-yl)-3-(tert-butyl)urea | + |
| 32 | 1-(1-benzyl-3-isopropyl-2-oxo-1,2,3,4-tetrahydroquinolin-6-yl)-3- | + |
| | (tert-butyl)urea | |
| 33 | 1-(tert-butyl)-3-(4-(3-chlorobenzyl)-3-oxo-3,4-dihydro-2H- | +++ |
| | benzo[b][1,4]oxazin-7-yl)urea | |
| 34 | (R)-1-(tert-butyl)-3-(3-oxo-4-(1-phenylethyl)-3,4-dihydro-2H- | ++ |
| | benzo[b][1,4]oxazin-7-yl)urea | |
| 35 | (S)-1-(tert-butyl)-3-(3-oxo-4-(1-phenylethyl)-3,4-dihydro-2H- | +++ |
| | benzo[b][1,4]oxazin-7-yl)urea | |
| 36 | (R)-1-(tert-butyl)-3-(4-(1-(3-chlorophenyl)ethyl)-3-oxo-3,4- | + |
| | dihydro-2H-benzo[b][1,4]oxazin-7-yl)urea | |
| 37 | (S)-1-(tert-butyl)-3-(4-(1-(3-chlorophenyl)ethyl)-3-oxo-3,4-dihydro- | +++ |
| | 2H-benzo[b][1,4]oxazin-7-yl)urea | |
| 38 | 1-(3-benzyl-2-oxo-2,3-dihydrobenzo[d]oxazol-6-yl)-3-(tert- | + |
| | butyl)urea | |
| 39 | 1-(tert-butyl)-3-(1-(2-fluoro-5-(trifluoromethyl)benzyl)-2-oxo- | +++ |
| | 1,2,3,4-tetrahydroquinolin-6-yl)urea | |
| 40 | 1-(tert-butyl)-3-(4-(3-methylbenzyl)-3-oxo-3,4-dihydro-2H- | +++ |
| | benzo[b][1,4]oxazin-7-yl)urea | |
| 41 | (R)-1-(4-benzyl-2-methyl-3-oxo-3,4-dihydro-2H- | +++ |
| | benzo[b][1,4]oxazin-7-yl)-3-(tert-butyl)urea | |
| 42 | (S)-1-(4-benzyl-2-methyl-3-oxo-3,4-dihydro-2H- | +++ |
| | benzo[b][1,4]oxazin-7-yl)-3-(tert-butyl)urea | |
| 43 | (R)-1-(tert-butyl)-3-(4-(3-chlorobenzyl)-2-methyl-3-oxo-3,4- | +++ |
| | dihydro-2H-benzo[b][1,4]oxazin-7-yl)urea | |
| 44 | (S)-1-(tert-butyl)-3-(4-(3-chlorobenzyl)-2-methyl-3-oxo-3,4- | +++ |
| | dihydro-2H-benzo[b][1,4]oxazin-7-yl)urea | |
| 45 | (S)-1-(tert-butyl)-3-(3-methyl-2-oxo-1-(1-phenylethyl)-1,2,3,4- | +++ |
| | tetrahydroquinazolin-6-yl)urea | |
| 45A | (R)-1-(tert-butyl)-3-(3-methyl-2-oxo-1-(1-phenylethyl)-1,2,3,4- | + |
| | tetrahydroquinazolin-6-yl)urea | |
| 46 | (S)-1-(tert-butyl)-3-(1-(1-(3-chlorophenyl)ethyl)-3-methyl-2-oxo- | +++ |
| | 1,2,3,4-tetrahydroquinazolin-6-yl)urea | |
| 47 | 1-(tert-butyl)-3-(1-(3-chlorobenzyl)-3-methyl-2-oxo-1,2,3,4- | +++ |
| | tetrahydroquinazolin-6-yl)urea | |
| 48 | 1-(tert-butyl)-3-(1-(2-fluoro-3-methylbenzyl)-2-oxo-1,2,3,4- | +++ |
| | tetrahydroquinolin-6-yl)urea | |
| 49 | 1-(tert-butyl)-3-(1-(2-fluoro-5-methylbenzyl)-2-oxo-1,2,3,4- | +++ |
| | tetrahydroquinolin-6-yl)urea | |
| 50 | 1-(tert-butyl)-3-(3-oxo-4-(3-(trifluoromethyl)benzyl)-3,4-dihydro- | +++ |
| | 2H-benzo[b][1,4]oxazin-7-yl)urea | |
| 51 | 1-(1-benzyl-3-(hydroxymethyl)-2-oxo-1,2,3,4-tetrahydroquinolin-6- | + |
| | yl)-3-(tert-butyl)urea | |
| 52 | (R)-1-(tert-butyl)-3-(1-(1-(3-chlorophenyl)ethyl)-3-methyl-2-oxo- | + |
| | 1,2,3,4-tetrahydroquinazolin-6-yl)urea | |
| 53 | 1-(tert-butyl)-3-(1-(2-cyano-3-methylbenzyl)-2-oxo-1,2,3,4- | ++ |
| | tetrahydroquinolin-6-yl)urea | |
| 54 | 1-(tert-butyl)-3-(1-(2-fluoro-3-(trifluoromethyl)benzyl)-2-oxo- | +++ |
| | 1,2,3,4-tetrahydroquinolin-6-yl)urea | |
| 55 | 1-(tert-butyl)-3-(1-(2-cyano-5-(trifluoromethyl)benzyl)-2-oxo- | +++ |
| | 1,2,3,4-tetrahydroquinolin-6-yl)urea | |
| 56 | (S)-1-(tert-butyl)-3-(2-oxo-1-(1-(m-tolyl)ethyl)-1,2,3,4- | +++ |
| | tetrahydroquinolin-6-yl)urea | |
| 57 | (S)-1-(tert-butyl)-3-(3-oxo-4-(1-(m-tolyl)ethyl)-3,4-dihydro-2H- | +++ |
| | benzo[b][1,4]oxazin-7-yl)urea | |
| 58 | (S)-1-(tert-butyl)-3-(1-(1-(2-fluorophenyl)ethyl)-2-oxo-1,2,3,4- | +++ |
| | tetrahydroquinolin-6-yl)urea | |
| 59 | 1-(tert-butyl)-3-(1-(2-cyano-3-(trifluoromethyl)benzyl)-2-oxo- | ++ |
| | 1,2,3,4-tetrahydroquinolin-6-yl)urea | |
| 60 | (S)-1-(tert-butyl)-3-(2-oxo-1-(1-(3-(trifluoromethyl)phenyl)ethyl)- | +++ |
| | 1,2,3,4-tetrahydroquinolin-6-yl)urea | |
| 61 | (S)-1-(tert-butyl)-3-(1-(1-(2-cyanophenyl)ethyl)-2-oxo-1,2,3,4- | ++ |
| | tetrahydroquinolin-6-yl)urea | |
| 62 | (S)-1-(tert-butyl)-3-(3-oxo-4-(1-(3-(trifluoromethyl)phenyl)ethyl)- | +++ |
| | 3,4-dihydro-2H-benzo[b][1,4]oxazin-7-yl)urea | |
| 63 | 1-benzyl-2-oxo-1,2,3,4-tetrahydroquinolin-6-yl tert-butylcarbamate | + |
| 64 | 1-(tert-butyl)-3-(1-(cyclohexylmethyl)-2-oxo-1,2,3,4- | + |
| | tetrahydroquinolin-6-yl)urea | |
| 65 | 1-(1-benzyl-7-fluoro-2-oxo-1,2,3,4-tetrahydroquinolin-6-yl)-3-(tert- | ++ |
| | butyl)urea | |
| 66 | 1-(1-benzyl-7-chloro-2-oxo-1,2,3,4-tetrahydroquinolin-6-yl)-3-(tert- | ++ |
| | butyl)urea | |
| 67 | 1-(5-benzyl-6-oxo-5,6,7,8-tetrahydro-1,5-naphthyridin-2-yl)-3-(tert- | ++ |
| | butyl)urea | |
| 68 | 1-(1-benzyl-2-oxo-1,2-dihydroquinoxalin-6-yl)-3-(tert-butyl)urea | +++ |
| 69 | 1-(1-benzyl-8-fluoro-2-oxo-1,2,3,4-tetrahydroquinolin-6-yl)-3-(tert- | ++ |
| | butyl)urea | |
| 70 | 1-(tert-butyl)-3-(3-methyl-2-oxo-1-(3-(trifluoromethyl)benzyl)- | +++ |
| | 1,2,3,4-tetrahydroquinazolin-6-yl)urea | |
| 71 | 1-(tert-butyl)-3-(4-(3-(difluoromethyl)benzyl)-3-oxo-3,4-dihydro- | ++ |
| | 2H-benzo[b][1,4]oxazin-7-yl)urea | |
| 72 | 1-(tert-butyl)-3-(4-(3-(hydroxymethyl)benzyl)-3-oxo-3,4-dihydro- | + |
| | 2H-benzo[b][1,4]oxazin-7-yl)urea | |
| 73 | 1-(tert-butyl)-3-(4-(3-cyclopropylbenzyl)-3-oxo-3,4-dihydro-2H- | ++ |
| | benzo[b][1,4]oxazin-7-yl)urea | |
| 74 | 1-(tert-butyl)-3-(3-oxo-4-(3-(trifluoromethoxy)benzyl)-3,4-dihydro- | ++ |
| | 2H-benzo[b][1,4]oxazin-7-yl)urea | |
| 75 | 1-(tert-butyl)-3-(4-(3-isopropoxybenzyl)-3-oxo-3,4-dihydro-2H- | ++ |
| | benzo[b][1,4]oxazin-7-yl)urea | |
| 76 | 1-(4-((1H-indazol-7-yl)methyl)-3-oxo-3,4-dihydro-2H- | + |
| | benzo[b][1,4]oxazin-7-yl)-3-(tert-butyl)urea | |
| 77 | 1-(tert-butyl)-3-(3-oxo-4-(3-(2,2,2-trifluoro-1- | ++ |
| | hydroxyethyl)benzyl)-3,4-dihydro-2H-benzo[b][1,4]oxazin-7- | |
| | yl)urea | |
| 78 | (R)-1-(tert-butyl)-3-(3-oxo-4-(3-(2,2,2-trifluoro-1- | ++ |
| | hydroxyethyl)benzyl)-3,4-dihydro-2H-benzo[b][1,4]oxazin-7- | |
| | yl)urea | |
| 79 | (S)-1-(tert-butyl)-3-(3-oxo-4-(3-(2,2,2-trifluoro-1- | ++ |
| | hydroxyethyl)benzyl)-3,4-dihydro-2H-benzo[b][1,4]oxazin-7- | |
| | yl)urea | |
| 80 | 1-(tert-butyl)-3-(3-oxo-4-(3-(3,3,3-trifluoro-1- | ++ |
| | hydroxypropyl)benzyl)-3,4-dihydro-2H-benzo[b][1,4]oxazin-7- | |
| | yl)urea | |
| 81 | (R)-1-(tert-butyl)-3-(2-methyl-3-oxo-4-(3-(trifluoromethyl)benzyl)- | +++ |
| | 3,4-dihydro-2H-benzo[b][1,4]oxazin-7-yl)urea | |
| 82 | (S)-1-(tert-butyl)-3-(2-methyl-3-oxo-4-(3-(trifluoromethyl)benzyl)- | ++ |
| | 3,4-dihydro-2H-benzo[b][1,4]oxazin-7-yl)urea | |
| 83 | (R)-1-(tert-butyl)-3-(4-(2-cyano-5-(trifluoromethyl)benzyl)-2- | + |
| | methyl-3-oxo-3,4-dihydro-2H-benzo[b][1,4]oxazin-7-yl)urea | |
| 84 | (S)-1-(tert-butyl)-3-(4-(2-cyano-5-(trifluoromethyl)benzyl)-2- | ++ |
| | methyl-3-oxo-3,4-dihydro-2H-benzo[b][1,4]oxazin-7-yl)urea | |
| 85 | (R)-1-(tert-butyl)-3-(4-(5-chloro-2-cyanobenzyl)-2-methyl-3-oxo- | ++ |
| | 3,4-dihydro-2H-benzo[b][1,4]oxazin-7-yl)urea | |
| 86 | (S)-1-(tert-butyl)-3-(4-(5-chloro-2-cyanobenzyl)-2-methyl-3-oxo- | + |
| | 3,4-dihydro-2H-benzo[b][1,4]oxazin-7-yl)urea | |
| 87 | (R)-1-(tert-butyl)-3-(4-(2-cyano-5-methylbenzyl)-2-methyl-3-oxo- | + |
| | 3,4-dihydro-2H-benzo[b][1,4]oxazin-7-yl)urea | |
| 88 | (S)-1-(tert-butyl)-3-(4-(2-cyano-5-methylbenzyl)-2-methyl-3-oxo- | + |
| | 3,4-dihydro-2H-benzo[b][1,4]oxazin-7-yl)urea | |
| 89 | 1-(tert-butyl)-3-(1-(3-chlorobenzyl)-2-oxo-1,2-dihydroquinoxalin-6- | ++ |
| | yl)urea | |
| 90 | 1-(tert-butyl)-3-(2-oxo-1-(3-(trifluoromethyl)benzyl)-1,2- | ++ |
| | dihydroquinoxalin-6-yl)urea | |
| 91 | 1-(tert-butyl)-3-(2-oxo-1-(3-(trifluoromethoxy)benzyl)-1,2- | ++ |
| | dihydroquinoxalin-6-yl)urea | |
| 92 | 1-(tert-butyl)-3-(2-oxo-1-(3-(3,3,3-trifluoropropyl)benzyl)-1,2- | ++ |
| | dihydroquinoxalin-6-yl)urea | |
| 93 | 1-(tert-butyl)-3-(2-oxo-1-(3-(4,4,4-trifluorobutyl)benzyl)-1,2- | +++ |
| | dihydroquinoxalin-6-yl)urea | |
| 94 | 1-(tert-butyl)-3-(2-oxo-1-(3-(2,2,2-trifluoroethyl)benzyl)-1,2- | ++ |
| | dihydroquinoxalin-6-yl)urea | |
| 95 | 1-(tert-butyl)-3-(2-oxo-1-((6-(4,4,4-trifluorobutyl)pyridin-2- | ++ |
| | yl)methyl)-1,2-dihydroquinoxalin-6-yl)urea | |
| 96 | 1-(tert-butyl)-3-(1-(chroman-8-ylmethyl)-2-oxo-1,2- | ++ |
| | dihydroquinoxalin-6-yl)urea | |
| 97 | 1-(tert-butyl)-3-(1-(chroman-5-ylmethyl)-2-oxo-1,2- | ++ |
| | dihydroquinoxalin-6-yl)urea | |
| 98 | (S)-1-(tert-butyl)-3-(2-oxo-1-(1-phenylethyl)-1,2- | ++ |
| | dihydroquinoxalin-6-yl)urea | |
| 99 | (S)-1-(tert-butyl)-3-(2-oxo-1-(1-(3-(trifluoromethyl)phenyl)ethyl)- | ++ |
| | 1,2-dihydroquinoxalin-6-yl)urea | |
| 100 | (S)-1-(tert-butyl)-3-(2-oxo-1-(1-(3-(trifluoromethoxy)phenyl)ethyl)- | +++ |
| | 1,2-dihydroquinoxalin-6-yl)urea | |
| 101 | (S)-1-(tert-butyl)-3-(1-(1-(3,5-dichlorophenyl)ethyl)-2-oxo-1,2- | ++ |
| | dihydroquinoxalin-6-yl)urea | |
| 102 | (S)-1-(tert-butyl)-3-(2-oxo-1-(1-(3-(2,2,2- | ++ |
| | trifluoroethyl)phenyl)ethyl)-1,2-dihydroquinoxalin-6-yl)urea | |
| 103 | (S)-1-(tert-butyl)-3-(2-oxo-1-(1-(6-(trifluoromethyl)pyridin-2- | ++ |
| | yl)ethyl)-1,2-dihydroquinoxalin-6-yl)urea | |
| 104 | (S)-1-(tert-butyl)-3-(1-(1-(2-fluoro-5- | ++ |
| | (trifluoromethoxy)phenyl)ethyl)-2-oxo-1,2-dihydroquinoxalin-6- | |
| | yl)urea | |
| 105 | (S)-1-(tert-butyl)-3-(1-(1-(4-fluoro-3- | +++ |
| | (trifluoromethoxy)phenyl)ethyl)-2-oxo-1,2-dihydroquinoxalin-6- | |
| | yl)urea | |
| 106 | (S)-1-(1-methylcyclobutyl)-3-(2-oxo-1-(1-(6- | ++ |
| | (trifluoromethoxy)pyridin-2-yl)ethyl)-1,2-dihydroquinoxalin-6- | |
| | yl)urea | |
| 107 | (S)-1-(tert-butyl)-3-(1-(1-(3-isopentylphenyl)ethyl)-2-oxo-1,2- | ++ |
| | dihydroquinoxalin-6-yl)urea | |
| 108 | (S)-1-(tert-butyl)-3-(2-oxo-1-(1-(3-(4,4,4- | ++ |
| | trifluorobutyl)phenyl)ethyl)-1,2-dihydroquinoxalin-6-yl)urea | |
| 109 | (S)-1-(tert-butyl)-3-(2-oxo-1-(1-(3-(3,3,3- | ++ |
| | trifluoropropoxy)phenyl)ethyl)-1,2-dihydroquinoxalin-6-yl)urea | |
| 110 | (S)-1-(tert-butyl)-3-(2-oxo-1-(1-(3-(2- | ++ |
| | (trifluoromethoxy)ethyl)phenyl)ethyl)-1,2-dihydroquinoxalin-6- | |
| | yl)urea | |
| 111 | (S)-1-(tert-butyl)-3-(1-(1-(3-(cyclopentyloxy)phenyl)ethyl)-2-oxo- | + |
| | 1,2-dihydroquinoxalin-6-yl)urea | |
| 112 | (S)-1-(1-(1-([1,1′-biphenyl]-3-yl)ethyl)-2-oxo-1,2- | ++ |
| | dihydroquinoxalin-6-yl)-3-(tert-butyl)urea | |
| 113 | (S)-1-(tert-butyl)-3-(1-(1-(3-(3-hydroxy-3-methylbut-1-yn-1- | + |
| | yl)phenyl)ethyl)-2-oxo-1,2-dihydroquinoxalin-6-yl)urea | |
| 114 | (S)-1-(tert-butyl)-3-(2-oxo-1-(1-(3-phenoxyphenyl)ethyl)-1,2- | ++ |
| | dihydroquinoxalin-6-yl)urea | |
| 115 | (S)-1-(tert-butyl)-3-(1-(1-(3-(cyclohexyloxy)phenyl)ethyl)-2-oxo- | ++ |
| | 1,2-dihydroquinoxalin-6-yl)urea | |
| 116 | (S)-1-(tert-butyl)-3-(1-(1-(3-(3-hydroxy-3- | + |
| | methylbutyl)phenyl)ethyl)-2-oxo-1,2-dihydroquinoxalin-6-yl)urea | |
| 117 | (S)-1-(tert-butyl)-3-(2-oxo-1-(1-(3-((tetrahydro-2H-pyran-4- | + |
| | yl)methoxy)phenyl)ethyl)-1,2-dihydroquinoxalin-6-yl)urea | |
| 118 | (S)-1-(tert-butyl)-3-(1-(1-(3-(4-hydroxy-4- | + |
| | methylpentyl)phenyl)ethyl)-2-oxo-1,2-dihydroquinoxalin-6-yl)urea | |
| 119 | (S)-1-(1-(1-(3-(2-((1H-pyrazol-4-yl)oxy)ethyl)phenyl)ethyl)-2-oxo- | + |
| | 1,2-dihydroquinoxalin-6-yl)-3-(tert-butyl)urea | |
| 120 | (S)-N-(3-(1-(6-(3-(tert-butyl)ureido)-2-oxoquinoxalin-1(2H)- | + |
| | yl)ethyl)phenethyl)-2-hydroxy-N,2-dimethylpropanamide | |
| 121 | (S)-1-(tert-butyl)-3-(1-(1-(3-(2-(2-hydroxy-2- | + |
| | methylpropoxy)ethyl)phenyl)ethyl)-2-oxo-1,2-dihydroquinoxalin-6- | |
| | yl)urea | |
| 122 | (S)-1-(tert-butyl)-3-(1-(1-(3-(difluoromethyl)phenyl)ethyl)-2-oxo- | ++ |
| | 1,2-dihydroquinoxalin-6-yl)urea | |
| 123 | (S)-1-(tert-butyl)-3-(1-(1-(isochroman-8-yl)ethyl)-2-oxo-1,2- | ++ |
| | dihydroquinoxalin-6-yl)urea | |
| 124 | (S)-1-(tert-butyl)-3-(1-(1-(isochroman-5-yl)ethyl)-2-oxo-1,2- | + |
| | dihydroquinoxalin-6-yl)urea | |
| 125 | (S)-1-(tert-butyl)-3-(1-(1-(2,3-dihydro-1H-inden-4-yl)ethyl)-2-oxo- | ++ |
| | 1,2-dihydroquinoxalin-6-yl)urea | |
| 126 | (S)-1-(tert-butyl)-3-(1-(1-(isoquinolin-8-yl)ethyl)-2-oxo-1,2- | + |
| | dihydroquinoxalin-6-yl)urea | |
| 127 | (S)-1-(tert-butyl)-3-(2-oxo-1-(1-(3-(piperidin-1-yl)phenyl)ethyl)- | ++ |
| | 1,2-dihydroquinoxalin-6-yl)urea | |
| 128 | (S)-1-(tert-butyl)-3-(1-(1-(3-(difluoromethoxy)phenyl)ethyl)-2-oxo- | ++ |
| | 1,2-dihydroquinoxalin-6-yl)urea | |
| 129 | (S)-1-(tert-butyl)-3-(2-oxo-1-(1-(4-(trifluoromethoxy)pyridin-2- | + |
| | yl)ethyl)-1,2-dihydroquinoxalin-6-yl)urea | |
| 130 | (S)-1-(tert-butyl)-3-(1-(1-(2,5-dichlorophenyl)ethyl)-2-oxo-1,2- | ++ |
| | dihydroquinoxalin-6-yl)urea | |
| 131 | (S)-1-(tert-butyl)-3-(1-(1-(3-chlorophenyl)ethyl)-2-oxo-1,2- | +++ |
| | dihydroquinoxalin-6-yl)urea | |
| 132 | (S)-1-(tert-butyl)-3-(2-oxo-1-(1-(m-tolyl)ethyl)-1,2- | ++ |
| | dihydroquinoxalin-6-yl)urea | |
| 133 | (S)-1-(tert-butyl)-3-(1-(1-(3,4-dichlorophenyl)ethyl)-2-oxo-1,2- | ++ |
| | dihydroquinoxalin-6-yl)urea | |
| 134 | (S)-1-(tert-butyl)-3-(1-(1-(3-cyclopropylphenyl)ethyl)-2-oxo-1,2- | ++ |
| | dihydroquinoxalin-6-yl)urea | |
| 135 | (S)-1-(tert-butyl)-3-(1-(1-(3-cyclobutylphenyl)ethyl)-2-oxo-1,2- | ++ |
| | dihydroquinoxalin-6-yl)urea | |
| 136 | (S)-1-(tert-butyl)-3-(1-(1-(3-isopropylphenyl)ethyl)-2-oxo-1,2- | ++ |
| | dihydroquinoxalin-6-yl)urea | |
| 137 | (S)-1-(tert-butyl)-3-(1-(1-(3-isopropoxyphenyl)ethyl)-2-oxo-1,2- | ++ |
| | dihydroquinoxalin-6-yl)urea | |
| 138 | (S)-1-(tert-butyl)-3-(1-(1-(3-(tert-butyl)phenyl)ethyl)-2-oxo-1,2- | ++ |
| | dihydroquinoxalin-6-yl)urea | |
| 139 | (S)-1-(tert-butyl)-3-(1-(1-(5-chloro-2-fluorophenyl)ethyl)-2-oxo- | ++ |
| | 1,2-dihydroquinoxalin-6-yl)urea | |
| 140 | (S)-1-(tert-butyl)-3-(1-(1-(2,6-difluorophenyl)ethyl)-2-oxo-1,2- | ++ |
| | dihydroquinoxalin-6-yl)urea | |
| 141 | (S)-1-(tert-butyl)-3-(1-(1-(5-chloro-2-methylphenyl)ethyl)-2-oxo- | ++ |
| | 1,2-dihydroquinoxalin-6-yl)urea | |
| 142 | (S)-1-(tert-butyl)-3-(1-(1-(2,3-dichlorophenyl)ethyl)-2-oxo-1,2- | ++ |
| | dihydroquinoxalin-6-yl)urea | |
| 143 | (S)-1-(tert-butyl)-3-(1-(1-(6-cyclopropylpyridin-2-yl)ethyl)-2-oxo- | ++ |
| | 1,2-dihydroquinoxalin-6-yl)urea | |
| 144 | (S)-1-(tert-butyl)-3-(1-(1-(6-methoxypyridin-2-yl)ethyl)-2-oxo-1,2- | ++ |
| | dihydroquinoxalin-6-yl)urea | |
| 145 | (S)-1-(tert-butyl)-3-(1-(1-(6-(dimethylamino)pyridin-2-yl)ethyl)-2- | ++ |
| | oxo-1,2-dihydroquinoxalin-6-yl)urea | |
| 146 | 1-(tert-butyl)-3-(1-((1S)-1-(3-(1-hydroxyethyl)phenyl)ethyl)-2-oxo- | + |
| | 1,2-dihydroquinoxalin-6-yl)urea | |
| 147 | (S)-1-(tert-butyl)-3-(1-(1-(2-chloro-5-fluorophenyl)ethyl)-2-oxo- | ++ |
| | 1,2-dihydroquinoxalin-6-yl)urea | |
| 148 | (S)-1-(tert-butyl)-3-(1-(1-(2,5-difluorophenyl)ethyl)-2-oxo-1,2- | ++ |
| | dihydroquinoxalin-6-yl)urea | |
| 149 | (S)-1-(tert-butyl)-3-(1-(1-(5-chloro-2,4-difluorophenyl)ethyl)-2- | ++ |
| | oxo-1,2-dihydroquinoxalin-6-yl)urea | |
| 150 | (S)-1-(tert-butyl)-3-(2-oxo-1-(1-(6-(2,2,2-trifluoroethyl)pyridin-2- | ++ |
| | yl)ethyl)-1,2-dihydroquinoxalin-6-yl)urea | |
| 151 | (S)-1-(tert-butyl)-3-(1-(1-(2,4-difluoro-5- | ++ |
| | (trifluoromethoxy)phenyl)ethyl)-2-oxo-1,2-dihydroquinoxalin-6- | |
| | yl)urea | |
| 152 | (S)-1-(tert-butyl)-3-(1-(1-(chroman-7-yl)ethyl)-2-oxo-1,2- | + |
| | dihydroquinoxalin-6-yl)urea | |
| 153 | 1-(tert-butyl)-3-(2-oxo-1-((S)-1-(6-((R)-2,2,2-trifluoro-1- | ++ |
| | hydroxyethyl)pyridin-2-yl)ethyl)-1,2-dihydroquinoxalin-6-yl)urea | |
| 154 | 1-(tert-butyl)-3-(2-oxo-1-((S)-1-(6-((S)-2,2,2-trifluoro-1- | ++ |
| | hydroxyethyl)pyridin-2-yl)ethyl)-1,2-dihydroquinoxalin-6-yl)urea | |
| 155 | 1-(tert-butyl)-3-(2-oxo-1-((S)-1-(3-((R)-2,2,2-trifluoro-1- | ++ |
| | hydroxyethyl)phenyl)ethyl)-1,2-dihydroquinoxalin-6-yl)urea | |
| 156 | 1-(tert-butyl)-3-(2-oxo-1-((S)-1-(3-((S)-2,2,2-trifluoro-1- | ++ |
| | hydroxyethyl)phenyl)ethyl)-1,2-dihydroquinoxalin-6-yl)urea | |
| 157 | (R)-1-(tert-butyl)-3-(2-methyl-4-(3-methylbenzyl)-3-oxo-3,4- | +++ |
| | dihydro-2H-benzo[b][1,4]oxazin-7-yl)urea | |
| 158 | (R)-1-(tert-butyl)-3-(4-(3-(difluoromethyl)benzyl)-2-methyl-3-oxo- | +++ |
| | 3,4-dihydro-2H-benzo[b][1,4]oxazin-7-yl)urea | |
| 159 | (R)-1-(tert-butyl)-3-(2-methyl-3-oxo-4-(3- | +++ |
| | (trifluoromethoxy)benzyl)-3,4-dihydro-2H-benzo[b][1,4]oxazin-7- | |
| | yl)urea | |
| 160 | (R)-1-(tert-butyl)-3-(4-(3-(difluoromethoxy)benzyl)-2-methyl-3- | +++ |
| | oxo-3,4-dihydro-2H-benzo[b][1,4]oxazin-7-yl)urea | |
| 161 | (R)-1-(tert-butyl)-3-(2-methyl-3-oxo-4-((6-(trifluoromethyl)pyridin- | +++ |
| | 2-yl)methyl)-3,4-dihydro-2H-benzo[b][1,4]oxazin-7-yl)urea | |
| 162 | (R)-1-(tert-butyl)-3-(2-methyl-3-oxo-4-((4-(trifluoromethyl)pyridin- | ++ |
| | 2-yl)methyl)-3,4-dihydro-2H-benzo[b][1,4]oxazin-7-yl)urea | |
| 163 | (R)-1-(tert-butyl)-3-(2-methyl-3-oxo-4-((2-(trifluoromethyl)pyridin- | ++ |
| | 4-yl)methyl)-3,4-dihydro-2H-benzo[b][1,4]oxazin-7-yl)urea | |
| 164 | (R)-1-(tert-butyl)-3-(4-(3-methoxybenzyl)-2-methyl-3-oxo-3,4- | ++ |
| | dihydro-2H-benzo[b][1,4]oxazin-7-yl)urea | |
| 165 | (R)-1-(tert-butyl)-3-(2-methyl-3-oxo-4-((2-(trifluoromethyl)thiazol- | ++ |
| | 4-yl)methyl)-3,4-dihydro-2H-benzo[b][1,4]oxazin-7-yl)urea | |
| 166 | (R)-1-(tert-butyl)-3-(2-methyl-3-oxo-4-((6- | +++ |
| | (trifluoromethoxy)pyridin-2-yl)methyl)-3,4-dihydro-2H- | |
| | benzo[b][1,4]oxazin-7-yl)urea | |
| 167 | (R)-1-(tert-butyl)-3-(2-methyl-3-oxo-4-((2- | ++ |
| | (trifluoromethyl)pyrimidin-4-yl)methyl)-3,4-dihydro-2H- | |
| | benzo[b][1,4]oxazin-7-yl)urea | |
| 168 | (R)-1-(tert-butyl)-3-(2-methyl-3-oxo-4-((4-(trifluoromethyl)thiazol- | ++ |
| | 2-yl)methyl)-3,4-dihydro-2H-benzo[b][1,4]oxazin-7-yl)urea | |
| 169 | (R)-1-(tert-butyl)-3-(2-methyl-3-oxo-4-((6-(trifluoromethyl)pyrazin- | ++ |
| | 2-yl)methyl)-3,4-dihydro-2H-benzo[b][1,4]oxazin-7-yl)urea | |
| 170 | (R)-1-(tert-butyl)-3-(2-methyl-3-oxo-4-((4- | ++ |
| | (trifluoromethyl)pyrimidin-2-yl)methyl)-3,4-dihydro-2H- | |
| | benzo[b][1,4]oxazin-7-yl)urea | |
| 171 | (R)-1-(tert-butyl)-3-(2-methyl-3-oxo-4-((5-(trifluoromethyl)pyridin- | ++ |
| | 3-yl)methyl)-3,4-dihydro-2H-benzo[b][1,4]oxazin-7-yl)urea | |
| 172 | 1-(tert-butyl)-3-((2R)-2-methyl-3-oxo-4-(3-(2,2,2-trifluoro-1- | +++ |
| | hydroxyethyl)benzyl)-3,4-dihydro-2H-benzo[b][1,4]oxazin-7- | |
| | yl)urea | |
| 173 | 1-(tert-butyl)-3-((2R)-2-methyl-3-oxo-4-((6-(2,2,2-trifluoro-1- | ++ |
| | hydroxyethyl)pyridin-2-yl)methyl)-3,4-dihydro-2H- | |
| | benzo[b][1,4]oxazin-7-yl)urea | |
| 174 | 1-(tert-butyl)-3-((R)-2-methyl-3-oxo-4-(3-((R)-2,2,2-trifluoro-1- | ++ |
| | hydroxyethyl)benzyl)-3,4-dihydro-2H-benzo[b][1,4]oxazin-7- | |
| | yl)urea | |
| 175 | 1-(tert-butyl)-3-((R)-2-methyl-3-oxo-4-(3-((S)-2,2,2-trifluoro-1- | ++ |
| | hydroxyethyl)benzyl)-3,4-dihydro-2H-benzo[b][1,4]oxazin-7- | |
| | yl)urea | |
| 176 | (R)-1-(tert-butyl)-3-(4-(2,5-dichlorobenzyl)-2-methyl-3-oxo-3,4- | +++ |
| | dihydro-2H-benzo[b][1,4]oxazin-7-yl)urea | |
| 177 | (R)-1-(tert-butyl)-3-(4-(2,5-difluorobenzyl)-2-methyl-3-oxo-3,4- | +++ |
| | dihydro-2H-benzo[b][1,4]oxazin-7-yl)urea | |
| 178 | (R)-1-(tert-butyl)-3-(4-(2-chlorobenzyl)-2-methyl-3-oxo-3,4- | ++ |
| | dihydro-2H-benzo[b][1,4]oxazin-7-yl)urea | |
| 179 | (S)-1-(tert-butyl)-3-(3-methyl-2-oxo-1-(1-phenylethyl)-1,2- | ++ |
| | dihydroquinoxalin-6-yl)urea | |
| 180 | (S)-1-(tert-butyl)-3-(1-(1-(3-chlorophenyl)ethyl)-3-methyl-2-oxo- | ++ |
| | 1,2-dihydroquinoxalin-6-yl)urea | |
| 181 | (S)-1-(tert-butyl)-3-(3-methyl-2-oxo-1-(1-(3- | ++ |
| | (trifluoromethyl)phenyl)ethyl)-1,2-dihydroquinoxalin-6-yl)urea | |
| 182 | (S)-1-(tert-butyl)-3-(3-methyl-2-oxo-1-(1-(3- | ++ |
| | (trifluoromethoxy)phenyl)ethyl)-1,2-dihydroquinoxalin-6-yl)urea | |
| 183 | 1-(tert-butyl)-3-((R)-4-((S)-1-(3-chlorophenyl)ethyl)-2-methyl-3- | +++ |
| | oxo-3,4-dihydro-2H-benzo[b][1,4]oxazin-7-yl)urea | |
| 184 | 1-(tert-butyl)-3-((R)-2-methyl-3-oxo-4-((S)-1-(3- | ++ |
| | (trifluoromethoxy)phenyl)ethyl)-3,4-dihydro-2H- | |
| | benzo[b][1,4]oxazin-7-yl)urea | |
| 185 | 1-(tert-butyl)-3-((R)-4-((S)-1-(3-(difluoromethyl)phenyl)ethyl)-2- | +++ |
| | methyl-3-oxo-3,4-dihydro-2H-benzo[b][1,4]oxazin-7-yl)urea | |
| 186 | 1-(tert-butyl)-3-((R)-2-methyl-3-oxo-4-((S)-1-(6- | +++ |
| | (trifluoromethyl)pyridin-2-yl)ethyl)-3,4-dihydro-2H- | |
| | benzo[b][1,4]oxazin-7-yl)urea | |
| 187 | 1-(tert-butyl)-3-((R)-4-((S)-1-(3-methoxyphenyl)ethyl)-2-methyl-3- | ++ |
| | oxo-3,4-dihydro-2H-benzo[b][1,4]oxazin-7-yl)urea | |
| 188 | 1-(tert-butyl)-3-((R)-4-((S)-1-(2,5-difluorophenyl)ethyl)-2-methyl- | +++ |
| | 3-oxo-3,4-dihydro-2H-benzo[b][1,4]oxazin-7-yl)urea | |
| 189 | 1-(tert-butyl)-3-((R)-4-((S)-1-(2-chlorophenyl)ethyl)-2-methyl-3- | +++ |
| | oxo-3,4-dihydro-2H-benzo[b][1,4]oxazin-7-yl)urea | |
| 190 | 1-(tert-butyl)-3-((R)-4-((S)-1-(2,5-dichlorophenyl)ethyl)-2-methyl- | ++ |
| | 3-oxo-3,4-dihydro-2H-benzo[b][1,4]oxazin-7-yl)urea | |
| 191 | 1-(1-benzyl-2-oxo-1,2-dihydroquinoxalin-6-yl)-3-(1- | ++ |
| | methylcyclobutyl)urea | |
| 192 | N-(1-benzyl-2-oxo-1,2-dihydroquinoxalin-6-yl)-3,3- | + |
| | dimethylazetidine-1-carboxamide | |
| 193 | 1-(1-benzyl-2-oxo-1,2-dihydroquinoxalin-6-yl)-3-(1- | ++ |
| | methylcyclopentyl)urea | |
| 194 | N-(1-benzyl-2-oxo-1,2-dihydroquinoxalin-6-yl)-3,3- | + |
| | dimethylpyrrolidine-1-carboxamide | |
| 195 | 1-(1-benzyl-2-oxo-1,2-dihydroquinoxalin-6-yl)-3-(1-fluoro-2- | + |
| | methylpropan-2-yl)urea | |
| 196 | 1-(1-benzyl-2-oxo-1,2-dihydroquinoxalin-6-yl)-3-cyclopentylurea | + |
| 197 | N-(1-benzyl-2-oxo-1,2-dihydroquinoxalin-6- | + |
| | yl)cyclopentanecarboxamide | |
| 198 | 1-(1-benzyl-2-oxo-1,2-dihydroquinoxalin-6-yl)-3-(1,1,1-trifluoro-2- | ++ |
| | methylpropan-2-yl)urea | |
| 199 | 1-(1-benzyl-2-oxo-1,2-dihydroquinoxalin-6-yl)-3-(1,1-difluoro-2- | ++ |
| | methylpropan-2-yl)urea | |
| 200 | (S)-1-(1-methylcyclobutyl)-3-(2-oxo-1-(1-(3- | +++ |
| | (trifluoromethoxy)phenyl)ethyl)-1,2-dihydroquinoxalin-6-yl)urea | |
| 201 | (S)-1-neopentyl-3-(2-oxo-1-(1-(3-(trifluoromethoxy)phenyl)ethyl)- | ++ |
| | 1,2-dihydroquinoxalin-6-yl)urea | |
| 202 | (S)-N-(2-oxo-1-(1-(3-(trifluoromethoxy)phenyl)ethyl)-1,2- | +++ |
| | dihydroquinoxalin-6-yl)-3-(3-(trifluoromethyl)phenyl)propanamide | |
| 203 | (S)-1-(1-methylcyclopentyl)-3-(2-oxo-1-(1-(3- | ++ |
| | (trifluoromethoxy)phenyl)ethyl)-1,2-dihydroquinoxalin-6-yl)urea | |
| 204 | (S)-1-(3,3-difluorocyclobutyl)-3-(2-oxo-1-(1-(3- | ++ |
| | (trifluoromethoxy)phenyl)ethyl)-1,2-dihydroquinoxalin-6-yl)urea | |
| 205 | (S)-1-cyclobutyl-3-(2-oxo-1-(1-(3-(trifluoromethoxy)phenyl)ethyl)- | ++ |
| | 1,2-dihydroquinoxalin-6-yl)urea | |
| 206 | (S)-1-(bicyclo[1.1.1]pentan-1-yl)-3-(2-oxo-1-(1-(3- | ++ |
| | (trifluoromethoxy)phenyl)ethyl)-1,2-dihydroquinoxalin-6-yl)urea | |
| 207 | (S)-1-isopentyl-3-(2-oxo-1-(1-(3-(trifluoromethoxy)phenyl)ethyl)- | ++ |
| | 1,2-dihydroquinoxalin-6-yl)urea | |
| 208 | (S)-1-(2-oxo-1-(1-(3-(trifluoromethoxy)phenyl)ethyl)-1,2- | ++ |
| | dihydroquinoxalin-6-yl)-3-(1,1,1-trifluoro-2-methylpropan-2- | |
| | yl)urea | |
| 209 | (S)-1-(tert-butyl)-3-(5-methyl-2-oxo-1-(1-(3- | ++ |
| | (trifluoromethoxy)phenyl)ethyl)-1,2-dihydroquinoxalin-6-yl)urea | |
| 210 | (S)-1-(1-methylcyclohexyl)-3-(2-oxo-1-(1-(3- | +++ |
| | (trifluoromethoxy)phenyl)ethyl)-1,2-dihydroquinoxalin-6-yl)urea | |
| 211 | (S)-1-(bicyclo[2.2.1]heptan-1-yl)-3-(2-oxo-1-(1-(3- | +++ |
| | (trifluoromethoxy)phenyl)ethyl)-1,2-dihydroquinoxalin-6-yl)urea | |
| 212 | (S)-1-(3-methyloxetan-3-yl)-3-(2-oxo-1-(1-(3- | + |
| | (trifluoromethoxy)phenyl)ethyl)-1,2-dihydroquinoxalin-6-yl)urea | |
| 213 | (S)-1-(bicyclo[2.1.1]hexan-1-yl)-3-(2-oxo-1-(1-(3- | +++ |
| | (trifluoromethoxy)phenyl)ethyl)-1,2-dihydroquinoxalin-6-yl)urea | |
| 214 | (S)-1-(2-oxo-1-(1-(3-(trifluoromethoxy)phenyl)ethyl)-1,2- | ++ |
| | dihydroquinoxalin-6-yl)-3-(1-(trifluoromethyl)cyclobutyl)urea | |
| 215 | (S)-1-(1-(methoxymethyl)cyclobutyl)-3-(2-oxo-1-(1-(3- | ++ |
| | (trifluoromethoxy)phenyl)ethyl)-1,2-dihydroquinoxalin-6-yl)urea | |
| 216 | (S)-1-(4,4-difluorocyclohexyl)-3-(2-oxo-1-(1-(3- | ++ |
| | (trifluoromethoxy)phenyl)ethyl)-1,2-dihydroquinoxalin-6-yl)urea | |
| 217 | (S)-1-(2-oxo-1-(1-(3-(trifluoromethoxy)phenyl)ethyl)-1,2- | ++ |
| | dihydroquinoxalin-6-yl)-3-(1-(trifluoromethyl)cyclopentyl)urea | |
| 218 | (S)-1-(tert-butyl)-3-(5-chloro-2-oxo-1-(1-(3- | ++ |
| | (trifluoromethoxy)phenyl)ethyl)-1,2-dihydroquinoxalin-6-yl)urea | |
| 219 | (R)-2,2-dimethyl-N-(2-oxo-1-((S)-1-(3- | +++ |
| | (trifluoromethoxy)phenyl)ethyl)-1,2-dihydroquinoxalin-6- | |
| | yl)cyclobutane-1-carboxamide | |
| 220 | (S)-2,2-dimethyl-N-(2-oxo-1-((S)-1-(3- | ++ |
| | (trifluoromethoxy)phenyl)ethyl)-1,2-dihydroquinoxalin-6- | |
| | yl)cyclobutane-1-carboxamide | |
| 221 | (S)-1-(3,3-difluoro-1-methylcyclobutyl)-3-(2-oxo-1-(1-(3- | ++ |
| | (trifluoromethoxy)phenyl)ethyl)-1,2-dihydroquinoxalin-6-yl)urea | |
| 222 | (S)-1-(2-oxo-1-(1-(3-(trifluoromethoxy)phenyl)ethyl)-1,2- | ++ |
| | dihydroquinoxalin-6-yl)-3-(3-(trifluoromethyl)oxetan-3-yl)urea | |
| 223 | 1-(2-oxo-1-((S)-1-(3-(trifluoromethoxy)phenyl)ethyl)-1,2- | ++ |
| | dihydroquinoxalin-6-yl)-3-((1r,3S)-3- | |
| | (trifluoromethyl)cyclobutyl)urea | |
| 224 | 1-(2-oxo-1-((S)-1-(3-(trifluoromethoxy)phenyl)ethyl)-1,2- | ++ |
| | dihydroquinoxalin-6-yl)-3-((1s,3R)-3- | |
| | (trifluoromethyl)cyclobutyl)urea | |
| 225 | (S)-1-(3,3-difluoro-1-(trifluoromethyl)cyclobutyl)-3-(2-oxo-1-(1-(3- | ++ |
| | (trifluoromethoxy)phenyl)ethyl)-1,2-dihydroquinoxalin-6-yl)urea | |
| 226 | (R)-1-(2-methyl-3-oxo-4-((6-(trifluoromethyl)pyridin-2-yl)methyl)- | +++ |
| | 3,4-dihydro-2H-benzo[b][1,4]oxazin-7-yl)-3-(1- | |
| | methylcyclobutyl)urea | |
| 227 | (R)-1-(2-methyl-3-oxo-4-((6-(trifluoromethyl)pyridin-2-yl)methyl)- | +++ |
| | 3,4-dihydro-2H-benzo[b][1,4]oxazin-7-yl)-3-(1- | |
| | methylcyclopentyl)urea | |
| 228 | (R)-1-cyclohexyl-3-(2-methyl-3-oxo-4-((6-(trifluoromethyl)pyridin- | +++ |
| | 2-yl)methyl)-3,4-dihydro-2H-benzo[b][1,4]oxazin-7-yl)urea | |
| 229 | (R)-1-(2-methyl-3-oxo-4-((6-(trifluoromethyl)pyridin-2-yl)methyl)- | +++ |
| | 3,4-dihydro-2H-benzo[b][1,4]oxazin-7-yl)-3-(1- | |
| | methylcyclohexyl)urea | |
| 230 | 1-(bicyclo[3.1.0]hexan-3-yl)-3-((R)-2-methyl-3-oxo-4-((6- | ++ |
| | (trifluoromethyl)pyridin-2-yl)methyl)-3,4-dihydro-2H- | |
| | benzo[b][1,4]oxazin-7-yl)urea | |
| 231 | (R)-1-(3,3-difluoro-1-methylcyclobutyl)-3-(2-methyl-3-oxo-4-((6- | ++ |
| | (trifluoromethyl)pyridin-2-yl)methyl)-3,4-dihydro-2H- | |
| | benzo[b][1,4]oxazin-7-yl)urea | |
| 232 | (R)-1-(2-methyl-3-oxo-4-((6-(trifluoromethyl)pyridin-2-yl)methyl)- | ++ |
| | 3,4-dihydro-2H-benzo[b][1,4]oxazin-7-yl)-3-(1- | |
| | (trifluoromethyl)cyclobutyl)urea | |
| 233 | (R)-1-(1-(methoxymethyl)cyclobutyl)-3-(2-methyl-3-oxo-4-((6- | ++ |
| | (trifluoromethyl)pyridin-2-yl)methyl)-3,4-dihydro-2H- | |
| | benzo[b][1,4]oxazin-7-yl)urea | |
| 234 | (R)-1-(2-methyl-3-oxo-4-((6-(trifluoromethyl)pyridin-2-yl)methyl)- | +++ |
| | 3,4-dihydro-2H-benzo[b][1,4]oxazin-7-yl)-3-(1- | |
| | (trifluoromethyl)cyclopentyl)urea | |
| 235 | (R)-1-(4,4-difluorocyclohexyl)-3-(2-methyl-3-oxo-4-((6- | ++ |
| | (trifluoromethyl)pyridin-2-yl)methyl)-3,4-dihydro-2H- | |
| | benzo[b][1,4]oxazin-7-yl)urea | |
| 236 | (R)-1-(4-benzyl-2-methyl-3-oxo-3,4-dihydro-2H- | ++ |
| | benzo[b][1,4]oxazin-7-yl)-3-(4-hydroxy-2-methylbutan-2-yl)urea | |
| 237 | (R)-1-(4-benzyl-2-methyl-3-oxo-3,4-dihydro-2H- | +++ |
| | benzo[b][1,4]oxazin-7-yl)-3-cyclopentylurea | |
| 238 | 1-((R)-4-benzyl-2-methyl-3-oxo-3,4-dihydro-2H- | + |
| | benzo[b][1,4]oxazin-7-yl)-3-(2-((1s,3S)-3- | |
| | hydroxycyclobutyl)propan-2-yl)urea | |
| 239 | 1-(2-hydroxy-2-methylpropyl)-3-(2-oxo-1-(3- | + |
| | (trifluoromethoxy)benzyl)-1,2-dihydroquinoxalin-6-yl)urea | |
| 240 | 1-neopentyl-3-(2-oxo-1-(3-(trifluoromethoxy)benzyl)-1,2- | ++ |
| | dihydroquinoxalin-6-yl)urea | |
| 241 | 1-(2-cyano-2-methylpropyl)-3-(2-oxo-1-(3- | + |
| | (trifluoromethoxy)benzyl)-1,2-dihydroquinoxalin-6-yl)urea | |
| 242 | 1-(3-hydroxy-2,2-dimethylpropyl)-3-(2-oxo-1-(3- | + |
| | (trifluoromethoxy)benzyl)-1,2-dihydroquinoxalin-6-yl)urea | |
| 243 | 1-(2-oxo-1-(3-(trifluoromethoxy)benzyl)-1,2-dihydroquinoxalin-6- | ++ |
| | yl)-3-(4,4,4-trifluoro-2-methylbutan-2-yl)urea | |
| 244 | 1-(2-oxo-1-(3-(trifluoromethoxy)benzyl)-1,2-dihydroquinoxalin-6- | ++ |
| | yl)-3-(3,3,3-trifluoro-2,2-dimethylpropyl)urea | |
| 245 | 1-(1-cyano-2-methylpropan-2-yl)-3-(2-oxo-1-(3- | + |
| | (trifluoromethoxy)benzyl)-1,2-dihydroquinoxalin-6-yl)urea | |
| 246 | 1-(tert-butyl)-3-(1-(1-(3-cyclobutylphenyl)ethyl)-2-oxo-1,2- | ++ |
| | dihydroquinoxalin-6-yl)urea | |
| 247 | 1-(tert-butyl)-3-(1-(1-(3-fluoro-5-(trifluoromethoxy)phenyl)ethyl)- | ++ |
| | 2-oxo-1,2-dihydroquinoxalin-6-yl)urea | |
| 248 | 1-(tert-butyl)-3-(1-(1-(3-(2-(oxetan-3-yl)ethyl)phenyl)ethyl)-2-oxo- | + |
| | 1,2-dihydroquinoxalin-6-yl)urea | |
| 249 | 1-(tert-butyl)-3-(2-oxo-1-(1-(1-(3,3,3-trifluoropropyl)-1H-indazol- | + |
| | 6-yl)ethyl)-1,2-dihydroquinoxalin-6-yl)urea | |
| 250 | 1-(tert-butyl)-3-(2-oxo-1-(1-(3-(2-(pyridin-4- | + |
| | ylmethoxy)ethyl)phenyl)ethyl)-1,2-dihydroquinoxalin-6-yl)urea | |
| 251 | 1-(tert-butyl)-3-(2-oxo-1-(1-(1-(3,3,3-trifluoropropyl)-1H-indazol- | + |
| | 4-yl)ethyl)-1,2-dihydroquinoxalin-6-yl)urea | |
| 252 | 1-(tert-butyl)-3-(2-oxo-1-(1-(quinolin-5-yl)ethyl)-1,2- | + |
| | dihydroquinoxalin-6-yl)urea | |
| 253 | 1-(tert-butyl)-3-(2-oxo-1-(1-(4-(trifluoromethyl)pyridin-2-yl)ethyl)- | + |
| | 1,2-dihydroquinoxalin-6-yl)urea | |
| 254 | 1-(tert-butyl)-3-(1-(1-(3,6-dichloropyridin-2-yl)ethyl)-2-oxo-1,2- | ++ |
| | dihydroquinoxalin-6-yl)urea | |
| 255 | 1-((R)-2-methyl-3-oxo-4-((S)-1-phenylethyl)-3,4-dihydro-2H- | +++ |
| | benzo[b][1,4]oxazin-7-yl)-3-(1-methylcyclobutyl)urea | |
| 256 | 1-((R)-2-methyl-3-oxo-4-((S)-1-phenylethyl)-3,4-dihydro-2H- | +++ |
| | benzo[b][1,4]oxazin-7-yl)-3-(1-methylcyclopentyl)urea | |
| 257 | 1-((R)-2-methyl-3-oxo-4-((S)-1-phenylethyl)-3,4-dihydro-2H- | +++ |
| | benzo[b][1,4]oxazin-7-yl)-3-(1-(trifluoromethyl)cyclopentyl)urea | |
| 258 | 1-(4,4-difluorocyclohexyl)-3-((R)-2-methyl-3-oxo-4-((S)-1- | ++ |
| | phenylethyl)-3,4-dihydro-2H-benzo[b][1,4]oxazin-7-yl)urea | |
| 259 | 1-((R)-2-methyl-3-oxo-4-((S)-1-phenylethyl)-3,4-dihydro-2H- | +++ |
| | benzo[b][1,4]oxazin-7-yl)-3-(1-(trifluoromethyl)cyclobutyl)urea | |
| 260 | 1-(3,3-difluoro-1-methylcyclobutyl)-3-((R)-2-methyl-3-oxo-4-((S)- | ++ |
| | 1-phenylethyl)-3,4-dihydro-2H-benzo[b][1,4]oxazin-7-yl)urea | |
| 261 | 1-(1-(methoxymethyl)cyclobutyl)-3-((R)-2-methyl-3-oxo-4-((S)-1- | ++ |
| | phenylethyl)-3,4-dihydro-2H-benzo[b][1,4]oxazin-7-yl)urea | |
| 262 | 1-((R)-2-methyl-3-oxo-4-((S)-1-phenylethyl)-3,4-dihydro-2H- | + |
| | benzo[b][1,4]oxazin-7-yl)-3-(3-methyloxetan-3-yl)urea | |
| 263 | 1-((R)-2-methyl-3-oxo-4-((S)-1-phenylethyl)-3,4-dihydro-2H- | ++ |
| | benzo[b][1,4]oxazin-7-yl)-3-(3-(trifluoromethyl)oxetan-3-yl)urea | |
| 264 | 1-(3,3-difluoro-1-(trifluoromethyl)cyclobutyl)-3-((R)-2-methyl-3- | ++ |
| | oxo-4-((S)-1-phenylethyl)-3,4-dihydro-2H-benzo[b][1,4]oxazin-7- | |
| | yl)urea | |
| 265 | 1-((R)-2-methyl-3-oxo-4-((S)-1-phenylethyl)-3,4-dihydro-2H- | ++ |
| | benzo[b][1,4]oxazin-7-yl)-3-(4-methyltetrahydro-2H-pyran-4- | |
| | yl)urea | |
| 266 | 1-(tert-butyl)-3-(1-(2-cyano-5-methylbenzyl)-2-oxo-1,2,3,4- | + |
| | tetrahydroquinolin-6-yl)urea | |
| 267 | 1-(1-benzyl-5-chloro-2-oxo-1,2,3,4-tetrahydroquinolin-6-yl)-3-(tert- | ++ |
| | butyl)urea | |
| 268 | 1-(tert-butyl)-3-((S)-2-methyl-3-oxo-4-((S)-1-phenylethyl)-3,4- | ++ |
| | dihydro-2H-benzo[b][1,4]oxazin-7-yl)urea | |
| 269 | 1-(tert-butyl)-3-((R)-2-methyl-3-oxo-4-((S)-1-phenylethyl)-3,4- | +++ |
| | dihydro-2H-benzo[b][1,4]oxazin-7-yl)urea | |
| 270 | 1-(1-benzyl-7-cyano-2-oxo-1,2,3,4-tetrahydroquinolin-6-yl)-3-(tert- | + |
| | butyl)urea | |
| 271 | 1-(tert-butyl)-3-(3-methyl-2-oxo-1-(1-(3- | + |
| | (trifluoromethyl)phenyl)ethyl)-1,2,3,4-tetrahydroquinazolin-6- | |
| | yl)urea | |
| 272 | 1-(1-benzyl-3-methyl-2-oxo-1,2-dihydroquinoxalin-6-yl)-3-(tert- | + |
| | butyl)urea | |
| 273 | 1-(tert-butyl)-3-(1-(3-chlorobenzyl)-3-methyl-2-oxo-1,2- | ++ |
| | dihydroquinoxalin-6-yl)urea | |
| 274 | 1-(tert-butyl)-3-(6-oxo-5-(3-(trifluoromethyl)benzyl)-5,6,7,8- | ++ |
| | tetrahydro-1,5-naphthyridin-2-yl)urea | |
| 275 | (R)-1-(1-benzyl-3-(cyanomethyl)-2-oxo-1,2,3,4-tetrahydroquinolin- | + |
| | 6-yl)-3-(tert-butyl)urea | |
| 276 | (S)-1-(1-benzyl-3-(cyanomethyl)-2-oxo-1,2,3,4-tetrahydroquinolin- | + |
| | 6-yl)-3-(tert-butyl)urea | |
| 277 | (R)-1-(4-benzyl-2-ethyl-3-oxo-3,4-dihydro-2H- | + |
| | benzo[b][1,4]oxazin-7-yl)-3-(tert-butyl)urea | |
| 278 | (S)-1-(4-benzyl-2-ethyl-3-oxo-3,4-dihydro-2H-benzo[b][1,4]oxazin- | + |
| | 7-yl)-3-(tert-butyl)urea | |
| 279 | 1-(1-benzyl-5-cyano-2-oxo-1,2,3,4-tetrahydroquinolin-6-yl)-3-(tert- | + |
| | butyl)urea | |
| 280 | (R)-1-(1-benzyl-3-methyl-2-oxo-2,3-dihydro-1H-pyrido[2,3- | ++ |
| | b][1,4]oxazin-6-yl)-3-(tert-butyl)urea | |
| 281 | 1-(tert-butyl)-3-(5-(3-chlorobenzyl)-6-oxo-5,6,7,8-tetrahydro-1,5- | ++ |
| | naphthyridin-2-yl)urea | |
| 282 | (S)-1-(tert-butyl)-3-(6-oxo-5-(1-(3-(trifluoromethyl)phenyl)ethyl)- | ++ |
| | 5,6,7,8-tetrahydro-1,5-naphthyridin-2-yl)urea | |
| 283 | (R)-1-(tert-butyl)-3-(6-oxo-5-(1-(3-(trifluoromethyl)phenyl)ethyl)- | + |
| | 5,6,7,8-tetrahydro-1,5-naphthyridin-2-yl)urea | |
| 284 | 1-(1-benzyl-2-oxo-1,2,3,4-tetrahydroquinolin-6-yl)-3-phenylurea | + |
| 285 | N-(1-benzyl-2-oxo-1,2,3,4-tetrahydroquinolin-6-yl)-3,3- | + |
| | dimethylbutanamide | |
| 286 | (S)-1-(tert-butyl)-3-(1-(3-chlorobenzyl)-3-methyl-2-oxo-2,3- | + |
| | dihydro-1H-pyrido[2,3-b][1,4]oxazin-6-yl)urea | |
| 287 | (R)-1-(tert-butyl)-3-(1-(3-chlorobenzyl)-3-methyl-2-oxo-2,3- | ++ |
| | dihydro-1H-pyrido[2,3-b][1,4]oxazin-6-yl)urea | |
| 288 | (S)-1-(4-benzyl-2-(methoxymethyl)-3-oxo-3,4-dihydro-2H- | + |
| | benzo[b][1,4]oxazin-7-yl)-3-(tert-butyl)urea | |
| 289 | (R)-1-(4-benzyl-2-(methoxymethyl)-3-oxo-3,4-dihydro-2H- | + |
| | benzo[b][1,4]oxazin-7-yl)-3-(tert-butyl)urea | |
| 290 | (S)-1-(tert-butyl)-3-(5-(1-(3-chlorophenyl)ethyl)-6-oxo-5,6,7,8- | + |
| | tetrahydro-1,5-naphthyridin-2-yl)urea | |
| 291 | (R)-1-(tert-butyl)-3-(5-(1-(3-chlorophenyl)ethyl)-6-oxo-5,6,7,8- | ++ |
| | tetrahydro-1,5-naphthyridin-2-yl)urea | |
| 292 | 1-(4-benzyl-2,2-dimethyl-3-oxo-3,4-dihydro-2H- | + |
| | benzo[b][1,4]oxazin-7-yl)-3-(tert-butyl)urea | |
| 293 | 1-(tert-butyl)-3-(4-(1-(5-chloro-2-cyanophenyl)ethyl)-3-oxo-3,4- | + |
| | dihydro-2H-benzo[b][1,4]oxazin-7-yl)urea | |
| 294 | 1-(3-amino-1-benzyl-2-oxo-1,2-dihydroquinoxalin-6-yl)-3-(tert- | + |
| | butyl)urea | |
| 295 | 1-(tert-butyl)-3-(3-ethyl-2-oxo-1-(3-(trifluoromethoxy)benzyl)-1,2- | + |
| | dihydroquinoxalin-6-yl)urea | |
| 296 | 1-(tert-butyl)-3-(3-oxo-4-(2,2,2-trifluoro-1-phenylethyl)-3,4- | ++ |
| | dihydro-2H-benzo[b][1,4]oxazin-7-yl)urea | |
| 297 | (S)-1-(tert-butyl)-3-(1-(1-(3-methoxyphenyl)ethyl)-2-oxo-1,2,3,4- | ++ |
| | tetrahydroquinolin-6-yl)urea | |
| 298 | 1-(tert-butyl)-3-(1-(chroman-4-yl)-2-oxo-1,2,3,4- | + |
| | tetrahydroquinolin-6-yl)urea | |
| 299 | (S)-N-(2-oxo-1-(1-(6-(trifluoromethyl)pyridin-2-yl)ethyl)-1,2- | ++ |
| | dihydroquinoxalin-6-yl)-3-(3-(trifluoromethyl)phenyl)propanamide | |
| 300 | (S)-4,4-dimethyl-N-(2-oxo-1-(1-(6-(trifluoromethyl)pyridin-2- | ++ |
| | yl)ethyl)-1,2-dihydroquinoxalin-6-yl)pentanamide | |
| 301 | 1-(1-benzyl-2-oxo-3-(trifluoromethyl)-1,2,3,4-tetrahydroquinolin-6- | + |
| | yl)-3-(tert-butyl)urea | |
| 302 | 1-(1-benzyl-2-oxo-1,2,3,4-tetrahydroquinazolin-6-yl)-3-(tert- | + |
| | butyl)urea | |
| 303 | cyclopentyl ((R)-2-methyl-3-oxo-4-((S)-1-phenylethyl)-3,4- | +++ |
| | dihydro-2H-benzo[b][1,4]oxazin-7-yl)carbamate | |
| 304 | cyclopentyl (R)-(2-methyl-3-oxo-4-((6-(trifluoromethyl)pyridin-2- | +++ |
| | yl)methyl)-3,4-dihydro-2H-benzo[b][1,4]oxazin-7-yl)carbamate | |
| 305 | 1-(4-benzyl-2-(fluoromethyl)-3-oxo-3,4-dihydro-2H- | ++ |
| | benzo[b][1,4]oxazin-7-yl)-3-(tert-butyl)urea | |
| 306 | 1-(tert-butyl)-3-((R)-2-methyl-3-oxo-4-((S)-1-(6- | + |
| | (trifluoromethyl)pyrazin-2-yl)ethyl)-3,4-dihydro-2H- | |
| | benzo[b][1,4]oxazin-7-yl)urea | |
| 307 | 1-(tert-butyl)-3-((R)-2-methyl-3-oxo-4-((R)-1-(6- | ++ |
| | (trifluoromethyl)pyrazin-2-yl)ethyl)-3,4-dihydro-2H- | |
| | benzo[b][1,4]oxazin-7-yl)urea | |
| 308 | N-(1-benzyl-2-oxo-1,2-dihydroquinoxalin-6-yl)-2,2- | ++ |
| | dimethylcyclopentane-1-carboxamide | |
| 309 | 1-(tert-butyl)-3-(1-(2,3-dihydro-1H-inden-1-yl)-2-oxo-1,2,3,4- | ++ |
| | tetrahydroquinolin-6-yl)urea | |
| 310 | 1-(tert-butyl)-3-((R)-2-methyl-3-oxo-4-((S)-1-(2- | ++ |
| | (trifluoromethyl)pyrimidin-4-yl)ethyl)-3,4-dihydro-2H- | |
| | benzo[b][1,4]oxazin-7-yl)urea | |
| 311 | 1-(tert-butyl)-3-((R)-2-methyl-3-oxo-4-((R)-1-(2- | + |
| | (trifluoromethyl)pyrimidin-4-yl)ethyl)-3,4-dihydro-2H- | |
| | benzo[b][1,4]oxazin-7-yl)urea | |
| 312 | 1-(tert-butyl)-3-(1-(1-(2-fluoro-3-(trifluoromethoxy)phenyl)ethyl)- | ++ |
| | 2-oxo-1,2-dihydroquinoxalin-6-yl)urea | |
| 313 | (S)-1-(tert-butyl)-3-(2-oxo-1-(1-(3-(2,2,2- | + |
| | trifluoroethoxy)phenyl)ethyl)-1,2-dihydroquinoxalin-6-yl)urea | |
| 314 | (R)-1-(tert-butyl)-3-(2-oxo-1-(1-(3-(2,2,2- | ++ |
| | trifluoroethoxy)phenyl)ethyl)-1,2-dihydroquinoxalin-6-yl)urea | |
| 315 | (S)-N-(2-oxo-1-(1-(3-(trifluoromethoxy)phenyl)ethyl)-1,2- | ++ |
| | dihydroquinoxalin-6-yl)cyclopentanecarboxamide | |
| 316 | 1-(tert-butyl)-3-((R)-2-methyl-3-oxo-4-((S)-1-(4- | + |
| | (trifluoromethyl)pyrimidin-2-yl)ethyl)-3,4-dihydro-2H- | |
| | benzo[b][1,4]oxazin-7-yl)urea | |
| 317 | 1-(tert-butyl)-3-((R)-2-methyl-3-oxo-4-((R)-1-(4- | ++ |
| | (trifluoromethyl)pyrimidin-2-yl)ethyl)-3,4-dihydro-2H- | |
| | benzo[b][1,4]oxazin-7-yl)urea | |
| 318 | (S)-N-(2-oxo-1-(1-(6-(trifluoromethoxy)pyridin-2-yl)ethyl)-1,2- | ++ |
| | dihydroquinoxalin-6-yl)-3-(3-(trifluoromethyl)phenyl)propanamide | |
| 319 | (S)-1-(1-(1-(3-chlorophenyl)ethyl)-2-oxo-1,2-dihydroquinoxalin-6- | ++ |
| | yl)-3-(1-methylcyclobutyl)urea | |
| 320 | (S)-1-(1-methylcyclobutyl)-3-(2-oxo-1-(1-(3- | ++ |
| | (trifluoromethyl)phenyl)ethyl)-1,2-dihydroquinoxalin-6-yl)urea | |
| 321 | (R)-1-(2-methyl-3-oxo-4-((6-(trifluoromethoxy)pyridin-2- | +++ |
| | yl)methyl)-3,4-dihydro-2H-benzo[b][1,4]oxazin-7-yl)-3-(1- | |
| | methylcyclopentyl)urea | |
| 322 | 1-((R)-2-methyl-3-oxo-4-((S)-1-(6-(trifluoromethyl)pyridin-2- | +++ |
| | yl)ethyl)-3,4-dihydro-2H-benzo[b][1,4]oxazin-7-yl)-3-(1- | |
| | methylcyclopentyl)urea | |
| 323 | 1-((R)-2-methyl-3-oxo-4-((S)-1-(6-(trifluoromethoxy)pyridin-2- | +++ |
| | yl)ethyl)-3,4-dihydro-2H-benzo[b][1,4]oxazin-7-yl)-3-(1- | |
| | methylcyclopentyl)urea | |
| 324 | 1-((R)-2-methyl-3-oxo-4-((S)-1-(3-(trifluoromethoxy)phenyl)ethyl)- | ++ |
| | 3,4-dihydro-2H-benzo[b][1,4]oxazin-7-yl)-3-((1r,3R)-3- | |
| | (trifluoromethyl)cyclobutyl)urea | |
| 325 | 1-((R)-2-methyl-3-oxo-4-((S)-1-(3-(trifluoromethoxy)phenyl)ethyl)- | ++ |
| | 3,4-dihydro-2H-benzo[b][1,4]oxazin-7-yl)-3-((1s,3S)-3- | |
| | (trifluoromethyl)cyclobutyl)urea | |
| 326 | (R)-1-(4-benzyl-6-fluoro-2-methyl-3-oxo-3,4-dihydro-2H- | ++ |
| | benzo[b][1,4]oxazin-7-yl)-3-(tert-butyl)urea | |
| 327 | (R)-1-(tert-butyl)-3-(4-(3-(difluoromethyl)benzyl)-6-fluoro-2- | +++ |
| | methyl-3-oxo-3,4-dihydro-2H-benzo[b][1,4]oxazin-7-yl)urea | |
| 328 | 1-(tert-butyl)-3-((R)-6-fluoro-2-methyl-3-oxo-4-((S)-1- | ++ |
| | phenylethyl)-3,4-dihydro-2H-benzo[b][1,4]oxazin-7-yl)urea | |
| 329 | (R)-1-(4-benzyl-6,8-difluoro-2-methyl-3-oxo-3,4-dihydro-2H- | ++ |
| | benzo[b][1,4]oxazin-7-yl)-3-(tert-butyl)urea | |
| 330 | (R)-1-(4-benzyl-2-methyl-3-oxo-6-(trifluoromethyl)-3,4-dihydro- | +++ |
| | 2H-benzo[b][1,4]oxazin-7-yl)-3-(tert-butyl)urea | |
| 331 | 1-(tert-butyl)-3-((R)-2-methyl-3-oxo-4-((R)-1-phenylethyl)-3,4- | + |
| | dihydro-2H-benzo[b][1,4]oxazin-7-yl)urea | |
| 332 | (R)-1-(4-benzyl-2,6-dimethyl-3-oxo-3,4-dihydro-2H- | ++ |
| | benzo[b][1,4]oxazin-7-yl)-3-(tert-butyl)urea | |
| 333 | (S)-1-(1-benzyl-7-fluoro-3-methyl-2-oxo-1,2,3,4- | + |
| | tetrahydroquinolin-6-yl)-3-(tert-butyl)urea | |
| 334 | (R)-1-(1-benzyl-7-fluoro-3-methyl-2-oxo-1,2,3,4- | + |
| | tetrahydroquinolin-6-yl)-3-(tert-butyl)urea | |
| 335 | (R)-1-(tert-butyl)-3-(4-(3-(difluoromethoxy)benzyl)-6,8-difluoro-2- | +++ |
| | methyl-3-oxo-3,4-dihydro-2H-benzo[b][1,4]oxazin-7-yl)urea | |
| 336 | (R)-1-(tert-butyl)-3-(4-(3-(difluoromethyl)benzyl)-6,8-difluoro-2- | +++ |
| | methyl-3-oxo-3,4-dihydro-2H-benzo[b][1,4]oxazin-7-yl)urea | |
| 337 | 1-(1-methylcyclobutyl)-3-(2-oxo-1-(1-(4-(trifluoromethyl)pyridin- | ++ |
| | 2-yl)ethyl)-1,2-dihydroquinoxalin-6-yl)urea | |
| 338 | (R)-1-(tert-butyl)-3-(4-(3-(difluoromethoxy)benzyl)-6-fluoro-2- | ++ |
| | methyl-3-oxo-3,4-dihydro-2H-benzo[b][1,4]oxazin-7-yl)urea | |
| 339 | (S)-1-(tert-butyl)-3-(7-fluoro-2-oxo-1-(1-(3- | ++ |
| | (trifluoromethoxy)phenyl)ethyl)-1,2-dihydroquinoxalin-6-yl)urea | |
| 340 | (R)-1-(tert-butyl)-3-(4-(3-(difluoromethyl)benzyl)-5,6-difluoro-2- | ++ |
| | methyl-3-oxo-3,4-dihydro-2H-benzo[b][1,4]oxazin-7-yl)urea | |
| 341 | (R)-1-(4-benzyl-6-cyano-2-methyl-3-oxo-3,4-dihydro-2H- | ++ |
| | benzo[b][1,4]oxazin-7-yl)-3-(tert-butyl)urea | |
| 342 | (R)-1-(tert-butyl)-3-(6-cyano-4-(3-(difluoromethyl)benzyl)-2- | +++ |
| | methyl-3-oxo-3,4-dihydro-2H-benzo[b][1,4]oxazin-7-yl)urea | |
| 343 | (S)-1-(tert-butyl)-3-(2-oxo-1-(1-phenylethyl)-7-(trifluoromethyl)- | +++ |
| | 1,2,3,4-tetrahydroquinolin-6-yl)urea | |
| 344 | (S)-1-(tert-butyl)-3-(7-chloro-2-oxo-1-(1-phenylethyl)-1,2- | ++ |
| | dihydroquinoxalin-6-yl)urea | |
| 345 | 1-(1-benzyl-5,7-difluoro-2-oxo-1,2,3,4-tetrahydroquinolin-6-yl)-3- | ++ |
| | (tert-butyl)urea | |
| 346 | (S)-1-(tert-butyl)-3-(2-oxo-1-(1-phenylethyl)-7-(trifluoromethyl)- | ++ |
| | 1,2-dihydroquinoxalin-6-yl)urea | |
| 347 | (S)-1-(tert-butyl)-3-(7-methyl-2-oxo-1-(1-phenylethyl)-1,2- | ++ |
| | dihydroquinoxalin-6-yl)urea | |
| 348 | (S)-1-(tert-butyl)-3-(7-cyclopropyl-2-oxo-1-(1-phenylethyl)-1,2- | ++ |
| | dihydroquinoxalin-6-yl)urea | |
| 349 | 1-(tert-butyl)-3-((R)-2-methyl-3-oxo-4-((S)-1-phenylethyl)-6- | +++ |
| | (trifluoromethyl)-3,4-dihydro-2H-benzo[b][1,4]oxazin-7-yl)urea | |
| 350 | 1-(1-benzyl-2-oxo-7-(trifluoromethyl)-1,2,3,4-tetrahydroquinolin-6- | ++ |
| | yl)-3-(tert-butyl)urea | |
| 351 | (S)-1-(tert-butyl)-3-(5-(hydroxymethyl)-2-oxo-1-(1-(3- | + |
| | (trifluoromethoxy)phenyl)ethyl)-1,2-dihydroquinoxalin-6-yl)urea | |
| 352 | (S)-1-(tert-butyl)-3-(7-cyano-2-oxo-1-(1-(3- | ++ |
| | (trifluoromethoxy)phenyl)ethyl)-1,2-dihydroquinoxalin-6-yl)urea | |
| 353 | (S)-1-(tert-butyl)-3-(5-fluoro-2-oxo-1-(1-(3- | ++ |
| | (trifluoromethoxy)phenyl)ethyl)-1,2-dihydroquinoxalin-6-yl)urea | |
| 354 | (R)-1-(4-benzyl-8-fluoro-2-methyl-3-oxo-6-(trifluoromethyl)-3,4- | ++ |
| | dihydro-2H-benzo[b][1,4]oxazin-7-yl)-3-(tert-butyl)urea | |
| 355 | (R)-1-(1-benzyl-7-chloro-3-methyl-2-oxo-2,3-dihydro-1H- | ++ |
| | pyrido[2,3-b][1,4]oxazin-6-yl)-3-(tert-butyl)urea | |
| 356 | (R)-1-(tert-butyl)-3-(4-(3-(difluoromethyl)benzyl)-8-fluoro-2- | ++ |
| | methyl-3-oxo-6-(trifluoromethyl)-3,4-dihydro-2H- | |
| | benzo[b][1,4]oxazin-7-yl)urea | |
| 357 | (S)-1-(tert-butyl)-3-(1-(1-(3-chlorophenyl)ethyl)-7-fluoro-2-oxo- | ++ |
| | 1,2-dihydroquinoxalin-6-yl)urea | |
| 358 | (S)-1-(tert-butyl)-3-(1-(1-(3-chlorophenyl)ethyl)-2-oxo-7- | ++ |
| | (trifluoromethyl)-1,2-dihydroquinoxalin-6-yl)urea | |
| 359 | cyclopentyl (S)-(2-oxo-1-(1-(3-(trifluoromethoxy)phenyl)ethyl)-1,2- | +++ |
| | dihydroquinoxalin-6-yl)carbamate | |
| 360 | (S)-1-(tert-butyl)-3-(7-(hydroxymethyl)-2-oxo-1-(1-phenylethyl)- | + |
| | 1,2-dihydroquinoxalin-6-yl)urea | |
| 361 | (S)-1-(tert-butyl)-3-(1-(1-(3-chlorophenyl)ethyl)-7-cyano-2-oxo- | ++ |
| | 1,2-dihydroquinoxalin-6-yl)urea | |
| 362 | (R)-1-(1-benzyl-7-cyano-3-methyl-2-oxo-2,3-dihydro-1H- | ++ |
| | pyrido[2,3-b][1,4]oxazin-6-yl)-3-(tert-butyl)urea | |
| 363 | (S)-1-(tert-butyl)-3-(7-cyano-1-(1-(2,5-dichlorophenyl)ethyl)-2-oxo- | ++ |
| | 1,2-dihydroquinoxalin-6-yl)urea | |
| 364 | (S)-1-(tert-butyl)-3-(1-(1-(5-chloro-2-fluorophenyl)ethyl)-7-cyano- | ++ |
| | 2-oxo-1,2-dihydroquinoxalin-6-yl)urea | |
| 365 | (S)-1-(tert-butyl)-3-(7-chloro-1-(1-(2,5-dichlorophenyl)ethyl)-2- | +++ |
| | oxo-1,2-dihydroquinoxalin-6-yl)urea | |
| 366 | 1-(tert-butyl)-3-((R)-6-cyano-2-methyl-3-oxo-4-((S)-1- | +++ |
| | phenylethyl)-3,4-dihydro-2H-benzo[b][1,4]oxazin-7-yl)urea | |
| 367 | (R)-1-(tert-butyl)-3-(6-cyano-4-(2,5-dichlorobenzyl)-2-methyl-3- | ++ |
| | oxo-3,4-dihydro-2H-benzo[b][1,4]oxazin-7-yl)urea | |
| 368 | (R)-1-(1-benzyl-3-methyl-2-oxo-7-(trifluoromethyl)-2,3-dihydro- | +++ |
| | 1H-pyrido[2,3-b][1,4]oxazin-6-yl)-3-(tert-butyl)urea | |
| 369 | (R)-1-(4-benzyl-6-cyano-8-fluoro-2-methyl-3-oxo-3,4-dihydro-2H- | +++ |
| | benzo[b][1,4]oxazin-7-yl)-3-(tert-butyl)urea | |
| 370 | (R)-1-(tert-butyl)-3-(6-cyano-4-(3-(difluoromethyl)benzyl)-8- | +++ |
| | fluoro-2-methyl-3-oxo-3,4-dihydro-2H-benzo[b][1,4]oxazin-7- | |
| | yl)urea | |
| 371 | (R)-1-(tert-butyl)-3-(7-cyano-1-(3-(difluoromethyl)benzyl)-3- | +++ |
| | methyl-2-oxo-2,3-dihydro-1H-pyrido[2,3-b][1,4]oxazin-6-yl)urea | |
| 372 | (S)-1-(tert-butyl)-3-(7-chloro-1-(1-(5-chloro-2-fluorophenyl)ethyl)- | +++ |
| | 2-oxo-1,2-dihydroquinoxalin-6-yl)urea | |
| 373 | (R)-1-(tert-butyl)-3-(4-(5-chloro-2-fluorobenzyl)-6-cyano-2-methyl- | ++ |
| | 3-oxo-3,4-dihydro-2H-benzo[b][1,4]oxazin-7-yl)urea | |
| 374 | (R)-1-(tert-butyl)-3-(1-(3-(difluoromethyl)benzyl)-3-methyl-2-oxo- | +++ |
| | 7-(trifluoromethyl)-2,3-dihydro-1H-pyrido[2,3-b][1,4]oxazin-6- | |
| | yl)urea | |
| 375 | 1-(tert-butyl)-3-(1-(1-(2-fluoro-5-(2,2,2-trifluoro-1- | + |
| | hydroxyethyl)phenyl)ethyl)-2-oxo-1,2-dihydroquinoxalin-6-yl)urea | |
| 376 | 1-(tert-butyl)-3-((R)-3-methyl-2-oxo-1-((S)-1-phenylethyl)-7- | +++ |
| | (trifluoromethyl)-2,3-dihydro-1H-pyrido[2,3-b][1,4]oxazin-6- | |
| | yl)urea | |
| 377 | (S)-1-(tert-butyl)-3-(6-oxo-5-(1-phenylethyl)-3-(trifluoromethyl)- | +++ |
| | 5,6,7,8-tetrahydro-1,5-naphthyridin-2-yl)urea | |
| 378 | (R)-1-(1-benzyl-3-methyl-2-oxo-7-(trifluoromethyl)-2,3-dihydro- | ++ |
| | 1H-pyrido[2,3-b][1,4]oxazin-6-yl)-3-(tert-butyl)urea | |

*IC₅₀ bins: + >1 μM; ++ = 1 μM-100 nM; and +++ <100 nM

### INCORPORATION BY REFERENCE

All of the U.S. patents and U.S. and PCT patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A compound of Formula (I): 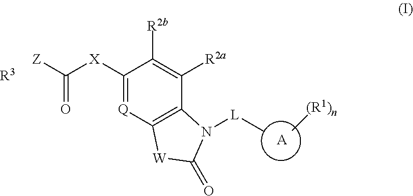
or a pharmaceutically acceptable salt thereof,
wherein:
A is (C₆-C₁₀)aryl, 5- to 10-membered heteroaryl, (C₃-C₈)cycloalkyl, or 4- to 7-membered heterocycloalkyl;
n is 0, 1, 2, 3, 4, or 5;
R¹ is independently for each occurrence (C₁-C₆)alkyl, (C₂-C₆)alkynyl, (C₂-C₆)alkenyl, (C₃-C₈)cycloalkyl, (C₃-C₈)cycloalkoxy, (C₆-C₁₀)aryl, (C₆-C₁₀)aryloxy, (C₆-C₁₀)aryl(C₁-C₆)alkyl, 4- to 7-membered heterocycloalkyl, (C₁-C₆)alkoxy, (C₁-C₆)alkoxy(C₁-C₆)alkyl, halo, cyano, amino, formyl, carboxy, or alkoxycarbonyl,
wherein (C₁-C₆)alkyl, (C₂-C₆)alkynyl, (C₂-C₆)alkenyl, (C₃-C₈)cycloalkyl, C₃-C₈)cycloalkoxy, (C₆-C₁₀)aryl, (C₆-C₁₀)aryloxy, (C₆-C₁₀)aryl(C₁-C₆)alkyl, (C₁-C₆)alkoxy, (C₁-C₆)alkoxy(C₁-C₆)alkyl, and 4- to 7-membered heterocycloalkyl are each optionally substituted with one or more substituents independently selected from halo, hydroxy, cyano, amido, sulfonamido, (C₁-C₆)alkyl, (C₃-C₈)cycloalkyl, 4- to 7-membered heterocycloalkyl, 5- to 6-membered heteroaryl, (C₁-C₆)alkoxy, (C₁-C₆)haloalkoxy, (C₁-C₆)fluoroalkoxy, 5- to 6-membered heteroaryloxy, or
two vicinal occurrences of R¹ taken together with the atoms to which they are attached form a fused 5- to 7-membered cycloalkyl, 5- to 7-membered heterocycloalkyl ring, 5- to 6-membered heteroaryl ring or phenyl ring, any of which is optionally substituted with one or more substituents independently selected from halo, hydroxy, cyano, amido, sulfonamido, (C₁-C₆)alkyl, (C₁-C₆)haloalkyl (C₃-C₈)cycloalkyl, and (C₁-C₆)alkoxy;
Q is N or CR^{2c};
R^{2a}, R^{2b}, and R^{2c}, are each independently hydrogen, halo, cyano, (C₁-C₆)alkyl, or (C₃-C₈)cycloalkyl, wherein (C₁-C₆)alkyl is optionally substituted with one or more substituents independently selected from halo, hydroxy, and (C₁-C₆)alkoxy;
R³ is (C₁-C₆)alkyl, (C₃-C₈)cycloalkyl, (C₃-C₈)cycloalkyl(C₁-C₆)alkyl), (C₆-C₁₀)aryl, (C₆-C₁₀)aryl(C₁-C₆)alkyl), or 4- to 7-membered heterocycloalkyl; each of which is optionally substituted with one or more substituents independently selected from halo, cyano, (C₁-C₆)haloalkyl, hydroxy, (C₁-C₆)alkoxy(C₁-C₆)alkyl, and (C₁-C₆)alkoxy;
X and Z are independently NR⁴, C(R⁵)₂, or 0;
R⁴ and R⁵ independently for each occurrence hydrogen or (C₁-C₆)alkyl, wherein (C₁-C₆)alkyl is optionally substituted with one or more substituents independently selected from halo, hydroxy, and (C₁-C₆)alkoxy, or when Z is NR⁴, R³ and R⁴ taken together with the nitrogen to which they are attached form a 4- to 7-member heterocycloalkyl optionally substituted with one or more (C₁-C₆)alkyl or halo;
L is (C₁-C₆)alkylene optionally substituted with one or more substituents independently selected from halo, hydroxy, C₁-C₆)alkoxy, and cyano;
W represents:
—C(R⁶)₂—C(R⁷)₂—,
—C(R⁷)₂—,
—O—C(R⁸)₂—,
—C(R⁸)₂—O—,
—C(R⁸)₂—NR⁹—,
—N═C(R¹⁰)—, or
—O—; and
R⁶, R⁷, R⁸, R⁹ and R¹⁰ are independently for each occurrence hydrogen, (C₁-C₆)alkyl, or NH₂, wherein (C₁-C₆)alkyl is optionally substituted with one or more substituents independently selected from halo, cyano, hydroxy, (C₁-C₆)alkoxy(C₁-C₆)alkyl, and (C₁-C₆)alkoxy.

2. A compound of Formula (I): 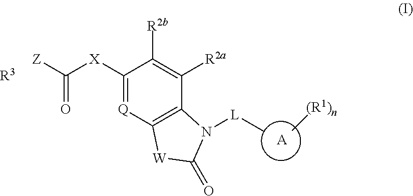
or a pharmaceutically acceptable salt thereof,
wherein:
A is (C₆-C₁₀)aryl or 5- to 10-membered heteroaryl;
n is 0, 1, 2, 3, 4, or 5;
R¹ is independently for each occurrence (C₁-C₆)alkyl, (C₃-C₈)cycloalkyl, 4- to 7-membered heterocycloalkyl, (C₁-C₆)alkoxy, halo, cyano, formyl, carboxy, or alkoxycarbonyl, wherein (C₁-C₆)alkyl, (C₃-C₈)cycloalkyl, (C₁-C₆)alkoxy, and 4- to 7-membered heterocycloalkyl are each optionally substituted with one or more substituents independently selected from halo, hydroxy, cyano, amido, sulfonamido, (C₁-C₆)alkyl, (C₃-C₈)cycloalkyl, and (C₁-C₆)alkoxy, or
two vicinal occurrences of R¹ taken together with the atoms to which they are attached form a fused 5- to 7-membered cycloalkyl or 5- to 7-membered heterocycloalkyl ring, either of which is optionally substituted with one or more substituents independently selected from halo, hydroxy, cyano, amido, sulfonamido, (C₁-C₆)alkyl, (C₃-C₈)cycloalkyl, and (C₁-C₆)alkoxy;
Q is N or CR^{2c};
R^{2a}, R^{2b}, and R^{2c}, are each independently hydrogen, halo, cyano, or (C₁-C₆)alkyl, wherein (C₁-C₆)alkyl is optionally substituted with one or more substituents independently selected from halo, hydroxy, and (C₁-C₆)alkoxy;
R³ is (C₁-C₆)alkyl optionally substituted with one or more substituents independently selected from halo, hydroxy, and (C₁-C₆)alkoxy;
X and Z are independently NR⁴, C(R⁵)₂, or 0;
R⁴ and R⁵ independently for each occurrence hydrogen or (C₁-C₆)alkyl, wherein (C₁-C₆)alkyl is optionally substituted with one or more substituents independently selected from halo, hydroxy, and (C₁-C₆)alkoxy;
L is (C₁-C₆)alkylene optionally substituted with one or more substituents independently selected from halo, hydroxy, C₁-C₆)alkoxy, and cyano;
W represents:
—C(R⁶)₂—C(R⁷)₂—,
—C(R⁷)₂—,
—O—C(R⁸)₂—,
—C(R⁸)₂—O—,
—C(R⁸)₂—NR⁹—,
—N═C(R¹⁰)—, or
—O—; and
R⁶, R⁷, R⁸, R⁹ and R¹⁰ are independently for each occurrence hydrogen or (C₁-C₆)alkyl, wherein (C₁-C₆)alkyl is optionally substituted with one or more substituents independently selected from halo, hydroxy, and (C₁-C₆)alkoxy.

3. A compound of Formula (II): 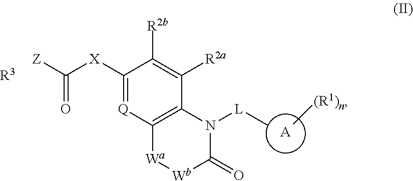
or a pharmaceutically acceptable salt thereof,
wherein
A is (C₆-C₁₀)aryl, 5- to 10-membered heteroaryl, (C₃-C₈)cycloalkyl, or 4- to 7-membered heterocycloalkyl;
n is 0, 1, 2, 3, 4, or 5;
R¹ is independently for each occurrence (C₁-C₆)alkyl, (C₂-C₆)alkynyl, (C₂-C₆)alkenyl, (C₃-C₈)cycloalkyl, (C₃-C₈)cycloalkoxy, (C₆-C₁₀)aryl, (C₆-C₁₀)aryloxy, (C₆-C₁₀)aryl(C₁-C₆)alkyl, 4- to 7-membered heterocycloalkyl, (C₁-C₆)alkoxy, (C₁-C₆)alkoxy(C₁-C₆)alkyl, halo, cyano, amino, formyl, carboxy, or alkoxycarbonyl,
wherein (C₁-C₆)alkyl, (C₂-C₆)alkynyl, (C₂-C₆)alkenyl, (C₃-C₈)cycloalkyl, C₃-C₈)cycloalkoxy, (C₆-C₁₀)aryl, (C₆-C₁₀)aryloxy, (C₆-C₁₀)aryl(C₁-C₆)alkyl, (C₁-C₆)alkoxy, (C₁-C₆)alkoxy(C₁-C₆)alkyl, and 4- to 7-membered heterocycloalkyl are each optionally substituted with one or more substituents independently selected from halo, hydroxy, cyano, amido, sulfonamido, (C₁-C₆)alkyl, (C₃-C₈)cycloalkyl, 4- to 7-membered heterocycloalkyl, 5- to 6-membered heteroaryl, (C₁-C₆)alkoxy, (C₁-C₆)haloalkoxy, (C₁-C₆)fluoroalkoxy, 5- to 6-membered heteroaryloxy, or
two vicinal occurrences of R¹ taken together with the atoms to which they are attached form a fused 5- to 7-membered cycloalkyl, 5- to 7-membered heterocycloalkyl ring, 5- to 6-membered heteroaryl ring or phenyl ring, any of which is optionally substituted with one or more substituents independently selected from halo, hydroxy, cyano, amido, sulfonamido, (C₁-C₆)alkyl, (C₁-C₆)haloalkyl (C₃-C₈)cycloalkyl, and (C₁-C₆)alkoxy;
Q is N or CR^{2c};
R^{2a}, R^{2b}, and R^{2c}, are each independently hydrogen, halo, cyano, (C₁-C₆)alkyl, or (C₃-C₈)cycloalkyl, wherein (C₁-C₆)alkyl is optionally substituted with one or more substituents independently selected from halo, hydroxy, and (C₁-C₆)alkoxy;
R³ is (C₁-C₆)alkyl, (C₃-C₈)cycloalkyl, (C₃-C₈)cycloalkyl(C₁-C₆)alkyl), (C₆-C₁₀)aryl, (C₆-C₁₀)aryl(C₁-C₆)alkyl), or 4- to 7-membered heterocycloalkyl; each of which is optionally substituted with one or more substituents independently selected from halo, cyano, (C₁-C₆)haloalkyl, hydroxy, (C₁-C₆)alkoxy(C₁-C₆)alkyl, and (C₁-C₆)alkoxy;
X and Z are independently NR⁴, C(R⁵)₂, or 0;
R⁴ and R⁵ independently for each occurrence hydrogen or (C₁-C₆)alkyl, wherein (C₁-C₆)alkyl is optionally substituted with one or more substituents independently selected from halo, hydroxy, and (C₁-C₆)alkoxy, or when Z is NR⁴, R³ and R⁴ taken together with the nitrogen to which they are attached form a 4- to 7-member heterocycloalkyl optionally substituted with one or more (C₁-C₆)alkyl or halo;
L is (C₁-C₆)alkylene optionally substituted with one or more substituents independently selected from halo, hydroxy, C₁-C₆)alkoxy, and cyano;
W represents:
C(R⁶)₂—C(R⁷)₂—,
—C(R⁷)₂—,
—O—C(R⁸)₂—,
—C(R⁸)₂—O—,
—C(R⁸)₂—NR⁹—,
—N═C(R¹⁰)—, or
—O—; and
R⁶, R⁷, R⁸, R⁹ and R¹⁰ are independently for each occurrence hydrogen, (C₁-C₆)alkyl, or NH₂, wherein (C₁-C₆)alkyl is optionally substituted with one or more substituents independently selected from halo, cyano, hydroxy, (C₁-C₆)alkoxy(C₁-C₆)alkyl, and (C₁-C₆)alkoxy.

4. A compound of Formula (II): 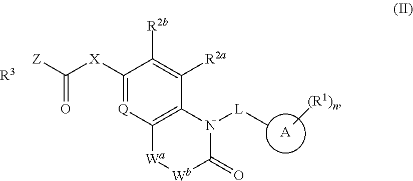
or a pharmaceutically acceptable salt thereof,
wherein
A is (C₆-C₁₀)aryl or 5- to 10-membered heteroaryl;
n is 0, 1, 2, 3, 4 or 5;
R¹ is independently for each occurrence (C₁-C₆)alkyl, (C₃-C₈)cycloalkyl, 4- to 7-membered heterocycloalkyl, (C₁-C₆)alkoxy, halo, cyano, formyl, carboxy, or alkoxycarbonyl, wherein (C₁-C₆)alkyl, (C₃-C₈)cycloalkyl, 4- to 7-membered heterocycloalkyl, and (C₁-C₆)alkoxy are each optionally substituted with one or more substituents independently selected from halo, hydroxy, cyano, amido, sulfonamido, (C₁-C₆)alkyl, (C₃-C₈)cycloalkyl, and (C₁-C₆)alkoxy; or
two vicinal occurrences of R¹ taken together with the atoms to which they are attached form a fused 5- to 7-membered cycloalkyl or 5- to 7-membered heterocycloalkyl ring, either of which is optionally substituted with one or more substituents independently selected from halo, hydroxy, cyano, amido, sulfonamido, (C₁-C₆)alkyl, (C₃-C₈)cycloalkyl, and (C₁-C₆)alkoxy;
Q is N or CR^{2c};
R^{2a}, R^{2b}, and R^{2c}, are each independently hydrogen, halo, cyano, or (C₁-C₆)alkyl, wherein (C₁-C₆)alkyl is optionally substituted with one or more substituents independently selected from halo, hydroxy, and (C₁-C₆)alkoxy;
R³ is (C₁-C₆)alkyl optionally substituted with one or more substituents independently selected from halo, hydroxy, and (C₁-C₆)alkoxy;
X and Z are independently NR⁴, C(R⁵)₂, or 0;
R⁴ and R⁵ are independently for each occurrence hydrogen or (C₁-C₆)alkyl, wherein (C₁-C₆)alkyl is optionally substituted with one or more substituents independently selected from halo, hydroxy, and (C₁-C₆)alkoxy;
L is (C₁-C₆)alkylene optionally substituted with one or more substituents independently selected from halo, hydroxy, C₁-C₆)alkoxy, and cyano;
W^{a}-W^{b} represents:
—C(R⁶)₂—C(R⁷)₂—,
—O—C(R⁸)₂—,
—C(R⁸)₂—O—,
—C(R⁸)₂—N⁹—, or
—N═C(R¹⁰)—; and
R⁶, R⁷, R⁸, R⁹ and R¹⁰ are independently for each occurrence hydrogen or (C₁-C₆)alkyl, wherein (C₁-C₆)alkyl is optionally substituted with one or more substituents independently selected from halo, hydroxy, and (C₁-C₆)alkoxy.

5. A compound of Formula (III): 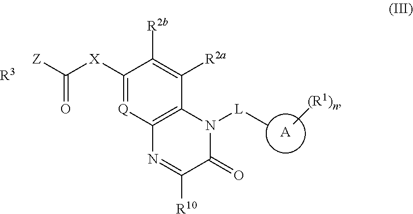
or a pharmaceutically acceptable salt thereof,
wherein:
A is (C₆-C₁₀)aryl, 5- to 10-membered heteroaryl, (C₃-C₈)cycloalkyl, or 4- to 7-membered heterocycloalkyl;
n is 0, 1, 2, 3, 4, or 5;
R¹ is independently for each occurrence (C₁-C₆)alkyl, (C₂-C₆)alkynyl, (C₂-C₆)alkenyl, (C₃-C₈)cycloalkyl, (C₃-C₈)cycloalkoxy, (C₆-C₁₀)aryl, (C₆-C₁₀)aryloxy, (C₆-C₁₀)aryl(C₁-C₆)alkyl, 4- to 7-membered heterocycloalkyl, (C₁-C₆)alkoxy, (C₁-C₆)alkoxy(C₁-C₆)alkyl, halo, cyano, amino, formyl, carboxy, or alkoxycarbonyl,
wherein (C₁-C₆)alkyl, (C₂-C₆)alkynyl, (C₂-C₆)alkenyl, (C₃-C₈)cycloalkyl, C₃-C₈)cycloalkoxy, (C₆-C₁₀)aryl, (C₆-C₁₀)aryloxy, (C₆-C₁₀)aryl(C₁-C₆)alkyl, (C₁-C₆)alkoxy, (C₁-C₆)alkoxy(C₁-C₆)alkyl, and 4- to 7-membered heterocycloalkyl are each optionally substituted with one or more substituents independently selected from halo, hydroxy, cyano, amido, sulfonamido, (C₁-C₆)alkyl, (C₃-C₈)cycloalkyl, 4- to 7-membered heterocycloalkyl, 5- to 6-membered heteroaryl, (C₁-C₆)alkoxy, (C₁-C₆)haloalkoxy, (C₁-C₆)fluoroalkoxy, 5- to 6-membered heteroaryloxy, or
two vicinal occurrences of R¹ taken together with the atoms to which they are attached form a fused 5- to 7-membered cycloalkyl, 5- to 7-membered heterocycloalkyl ring, 5- to 6-membered heteroaryl ring or phenyl ring, any of which is optionally substituted with one or more substituents independently selected from halo, hydroxy, cyano, amido, sulfonamido, (C₁-C₆)alkyl, (C₁-C₆)haloalkyl (C₃-C₈)cycloalkyl, and (C₁-C₆)alkoxy;
Q is N or CR^{2c};
R^{2a}, R^{2b}, and R^{2c}, are each independently hydrogen, halo, cyano, (C₁-C₆)alkyl, or (C₃-C₈)cycloalkyl, wherein (C₁-C₆)alkyl is optionally substituted with one or more substituents independently selected from halo, hydroxy, and (C₁-C₆)alkoxy;
R³ is (C₁-C₆)alkyl, (C₃-C₈)cycloalkyl, (C₃-C₈)cycloalkyl(C₁-C₆)alkyl), (C₆-C₁₀)aryl, (C₆-C₁₀)aryl(C₁-C₆)alkyl), or 4- to 7-membered heterocycloalkyl; each of which is optionally substituted with one or more substituents independently selected from halo, cyano, (C₁-C₆)alkyl, (C₁-C₆)haloalkyl, hydroxy, (C₁-C₆)alkoxy(C₁-C₆)alkyl, and (C₁-C₆)alkoxy;
X and Z are independently NR⁴, C(R⁵)₂, or 0;
R⁴ and R⁵ independently for each occurrence hydrogen or (C₁-C₆)alkyl, wherein (C₁-C₆)alkyl is optionally substituted with one or more substituents independently selected from halo, hydroxy, and (C₁-C₆)alkoxy, or when Z is NR⁴, R³ and R⁴ taken together with the nitrogen to which they are attached form a 4- to 7-member heterocycloalkyl optionally substituted with one or more (C₁-C₆)alkyl or halo, or when Z is C(R⁵)₂, R³ and one R⁵ taken together with the carbon to which they are attached form a 4, (C₃-C₈)cycloalkyl optionally substituted with one or more (C₁-C₆)alkyl or halo;
L is (C₁-C₆)alkylene optionally substituted with one or more substituents independently selected from halo, hydroxy, C₁-C₆)alkoxy, and cyano;
R¹⁰ is hydrogen, (C₁-C₆)alkyl, or NH₂, wherein (C₁-C₆)alkyl is optionally substituted with one or more substituents independently selected from halo, cyano, hydroxy, (C₁-C₆)alkoxy(C₁-C₆)alkyl, and (C₁-C₆)alkoxy.

6. A compound of Formula (III): 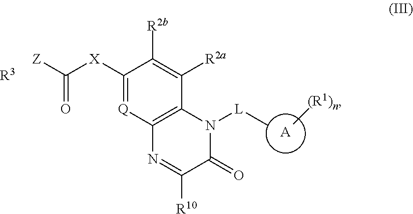
or a pharmaceutically acceptable salt thereof,
wherein
A is (C₆-C₁₀)aryl or 5- to 10-membered heteroaryl;
n is 0, 1, 2, 3, 4 or 5;
R¹ is independently for each occurrence (C₁-C₆)alkyl, (C₃-C₈)cycloalkyl, 4- to 7-membered heterocycloalkyl, (C₁-C₆)alkoxy, halo, cyano, formyl, carboxy, or alkoxycarbonyl, wherein (C₁-C₆)alkyl, (C₃-C₈)cycloalkyl, 4- to 7-membered heterocycloalkyl, and (C₁-C₆)alkoxy are each optionally substituted with one or more substituents independently selected from halo, hydroxy, cyano, amido, sulfonamido, (C₁-C₆)alkyl, (C₃-C₈)cycloalkyl, and (C₁-C₆)alkoxy; or
two vicinal occurrences of R¹ taken together with the atoms to which they are attached form a fused 5- to 7-membered cycloalkyl or 5- to 7-membered heterocycloalkyl ring, either of which is optionally substituted with one or more substituents independently selected from halo, hydroxy, cyano, amido, sulfonamido, (C₁-C₆)alkyl, (C₃-C₈)cycloalkyl, and (C₁-C₆)alkoxy;
Q is N or CR^{2c};
R^{2a}, R^{2b}, and R^{2c}, are each independently hydrogen, halo, cyano, (C₁-C₆)alkyl, wherein (C₁-C₆)alkyl is optionally substituted with one or more substituents independently selected from halo, hydroxy, and (C₁-C₆)alkoxy;
R³ is (C₁-C₆)alkyl optionally substituted with one or more substituents independently selected from halo, hydroxy, and (C₁-C₆)alkoxy;
X and Z are independently NR⁴, C(R⁵)₂, or 0;
R⁴ and R⁵ are independently for each occurrence hydrogen or (C₁-C₆)alkyl, wherein (C₁-C₆)alkyl is optionally substituted with one or more substituents independently selected from halo, hydroxy, and (C₁-C₆)alkoxy;
L is (C₁-C₆)alkylene optionally substituted with one or more substituents independently selected from halo, hydroxy, C₁-C₆)alkoxy, and cyano; and
R¹⁰ is hydrogen or (C₁-C₆)alkyl, wherein (C₁-C₆)alkyl is optionally substituted with one or more substituents independently selected from halo, hydroxy, and (C₁-C₆)alkoxy.

7. The compound of any one of claims 1 to 6, wherein A is phenyl.

8. The compound of any one of claims 1 to 6, wherein A is a 5- to 7-membered heteroaryl comprising 1 or 2 nitrogen atoms.

9. The compound of claim 8, wherein A is pyridyl or diazolyl, thiazolyl, pyrazinyl, pyrimidinyl,

10. The compound of claim 9, wherein A is pyridyl or diazolyl.

11. The compound of any one of claims 1 to 6, wherein A is isoquinolinyl or indazolyl.

12. The compound of any one of claims 1 to 6, wherein A is cyclopentyl, cyclohexyl, or tetrahydropyranyl.

13. The compound of any one of claims 1 to 12, wherein R¹ is independently for each occurrence (C₁-C₆)alkyl, (C₁-C₆)alkoxy, fluoro, chloro, or cyano, wherein (C₁-C₆)alkyl and (C₁-C₆)alkoxy are optionally substituted with one or more substituents independently selected from fluoro and hydroxyl.

14. The compound of any one of claims 1 to 12, wherein at least one R¹ is fluoro, chloro, or cyano.

15. The compound of any one of claims 1 to 14, wherein at least one R¹ is (C₁-C₆)alkyl optionally substituted with one or more substituents independently selected from hydroxyl and fluoro,

16. The compound claim 15, wherein at least one R¹ is 2,2,2-trifluoroethyl-1-hydroxyethyl.

17. The compound claim 15, wherein at least one R¹ wherein at least one R¹ is difluoromethyl, trifluoromethyl, 2,2,2-trifluoroethyl, 3,3,3-trifluropropyl, or 4,4,4-trifluorobutyl.

18. The compound of any one of claims 1 to 14, wherein at least one R¹ is (C₁-C₆)alkoxy optionally substituted with at least one fluoro.

19. The compound of claim 18, wherein at least one R¹ is difluoromethoxy or trifluoromethoxy.

20. The compound of claim 18, wherein at least one R¹ is methoxy or isopropoxy.

21. The compound of any one of claims 1 to 14, wherein at least one R¹ is methyl optionally substituted with hydroxy or one or more fluoro.

22. The compound of any one of claims 1 to 14, wherein at least R¹ is (C₁-C₆)alkyl substituted with an amido having the structure:

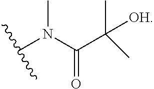 [SMILES: *N(C)C(=O)C(C)(C)O]

23. The compound of any one of claims 1 to 14, wherein at least one R¹ is (C₃-C₈)cycloalkyl.

24. The compound of claim 23, wherein the (C₃-C₈)cycloalkyl is cyclopropyl or cyclobutyl.

25. The compound of any one of claims 1 to 14, wherein at least R¹ is (C₃-C₈)cycloalkoxy.

26. The compound of claim 25, wherein the (C₃-C₈)cycloalkoxy is cyclopentyloxy or cyclohexyloxy.

27. The compound of any one of claims 1 to 14, wherein R¹ is (C₂-C₆)alkynyl optionally substituted with hydroxy.

28. The compound of any one of claims 1 to 12, wherein two vicinal occurrences of R¹ taken together with the atoms to which they are attached form a fused 5- to 7-membered cycloalkyl or 5- to 7-membered heterocycloalkyl ring, either of which is optionally substituted with one or more substituents independently selected from halo, hydroxy, cyano, amido, sulfonamido, (C₁-C₆)alkyl, (C₃-C₈)cycloalkyl, and (C₁-C₆)alkoxy.

29. The compound of claim 28, wherein A is phenyl comprising two vicinal occurrences of R¹, wherein the two vicinal occurrences of R¹, taken together with the atoms to which they are attached, form a fused 5- to 7-membered cycloalkyl or 5- to 7-membered heterocycloalkyl ring.

30. The compound of any one of claims 1 to 27, wherein n is 2.

31. The compound of any one of claims 1 to 27, wherein n is 1.

32. The compound of any one of claims 1 to 12, wherein n is 0.

33. The compound of any one of claims 1 to 32, wherein Q is CR^{2c}.

34. The compound of any one of claims 1 to 32, wherein Q is N.

35. The compound of any one of claims 1 to 33, wherein R^{2a}, R^{2b}, and R^{2C} are each hydrogen.

36. The compound of any one of claims 1 to 33, wherein at least one of R^{2a}, R^{2b}, and R^{2c} is fluoro, chloro, or cyano.

37. The compound of any one of claims 1 to 36, wherein R³ is (C₁-C₆)alkyl optionally substituted with hydroxy.

38. The compound of claim 37, wherein R³ is t-butyl.

39. The compound of any one of claims 1 to 36, wherein R³ is (C₃-C₈)cycloalkyl optionally substituted with one or more (C₁-C₆)alkyl, (C₁-C₆)fluoroalkyl, fluoro, or (C₁-C₆)alkoxy(C₁-C₆)alkyl.

40. The compound of claim 39, wherein the (C₃-C₈)cycloalkyl is cyclobutyl, cyclopentyl, cyclohexyl, bicyclo[1.1.1]pentanyl, bicyclo[2.2.1]heptanyl, bicycle[2.1.1]hexanyl, or bicyclo[3.1.0]hexanyl.

41. The compound of claim 40 or 41, wherein the (C₃-C₈)cycloalkyl is optionally substituted with one or more substituents selected from fluoro, trifluoromethyl, methoxymethyl, methyl, and hydroxyl.

42. The compound of any one of claims 1 to 36, wherein R³ is 4- to 7-membered heterocycloalkyl optionally substituted with one or more substituents selected from (C₁-C₆)alkyl, (C₁-C₆)fluoroalkyl, or fluoro.

43. The compound of claim 42, wherein the 4- to 7-membered heterocycloalkyl is tetrahydropyranyl or oxetanyl.

44. The compound of any one of claims 1 to 36, wherein Z is NR⁴, R³ and R⁴ taken together with the nitrogen to which they are attached form a 4- to 7-member heterocycloalkyl optionally substituted with one or more (C₁-C₆)alkyl or halo.

45. The compound of claim 44, wherein the 4- to 7 membered heterocycloalkyl is azetidine or pyrrolidine,

46. The compound of any one of claims 1 to 45, wherein X is NR⁴ or O.

47. The compound of any one of claims 1 to 45 wherein X is NH.

48. The compound of any one of claims 1 to 43 and 46 to 47, wherein Z is NR⁴, or O.

49. The compound of any one of claims 1 to 43 and 46 to 47, wherein Z is NH.

50. The compound of any one of claims 1 to 49, wherein one and only one of X and Z is CH₂.

51. The compound of any one of claims 1 to 43 and 46 to 47, wherein X and Z are each NH.

52. The compound of any one of claims 1 to 51, wherein L is unsubstituted (C₁-C₆)alkylene.

53. The compound of any one of claims 1 to 51, wherein L is (C₁-C₆)alkylene substituted with hydroxy.

54. The compound of any one of claims 1 to 51, wherein L is (C₁-C₆)alkylene substituted with 1 to 3 halo atoms.

55. The compound of any one of claims 1 to 51, wherein L is —CH₂—, —CH(CH₃)—, —CH(CH₂CH₃)—, or —CH(CH₂OH)—.

56. The compound of any one of claims 2 to 55, wherein
W^{a}-W^{b} represents:
—C(R⁶)₂—C(R⁷)₂—.

57. The compound of claim 56, wherein at least one R⁶ is (C₁-C₆)alkyl, wherein (C₁-C₆)alkyl is optionally substituted with one or more substituents independently selected from halo, hydroxy, and (C₁-C₆)alkoxy.

58. The compound of claim 56 or 57, wherein at least one R⁷ is (C₁-C₆)alkyl, wherein (C₁-C₆)alkyl is optionally substituted with one or more substituents independently selected from halo, hydroxy, (C₁-C₆)alkoxy and cyano.

59. The compound of claim 56, wherein each R⁶ and each R⁷ is hydrogen.

60. The compound of any one of claims 2 to 55, wherein:
W^{a}-W^{b} represents:
—O—C(R′)₂—.

61. The compound of claim 60, wherein each R⁸ is hydrogen.

62. The compound of claim 60, wherein one R⁸ is (C₁-C₆)alkyl; and one R⁸ is hydrogen.

63. The compound of claim 62, wherein one R⁸ is methyl.

64. The compound of any one of claims 2 to 55, wherein:
W^{a}-W^{b} represents:
—C(R′)₂—O—.

65. The compound of claim 64, wherein each R⁸ is hydrogen.

66. The compound of any one of claims 2 to 30, wherein:
W^{a}-W^{b} represents:
—C(R⁸)₂—NR⁹—.

67. The compound of claim 66, wherein each R⁸ is hydrogen.

68. The compound of claim 66 or 67, wherein R⁹ is (C₁-C₆)alkyl.

69. The compound of claim 68, wherein R⁹ is methyl.

70. The compound of any one of claims 2 to 55, wherein:
W^{a}-W^{b} represents:
—N═C(R¹⁰)—.

71. The compound of claim 70, wherein R¹⁰ is hydrogen.

72. The compound of claim 70, wherein R¹⁰ is (C₁-C₆)alkyl.

73. The compound of claim 70, wherein R¹⁰ is methyl.

74. A compound selected from the following table:

| | | |
|---|---|---|
| 1 | 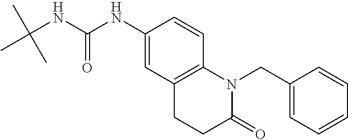 | 1-(1-benzyl-2-oxo-1,2,3,4-tetrahydroquinolin-6- yl)-3-(tert-butyl)urea |
| 2 | 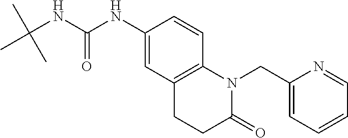 [SMILES: CC(C)(C)NC(=O)Nc1ccc2c(c1)CCC(=O)N2Cc1ccccn1] | 1-(tert-butyl)-3-(2-oxo-1-(pyridin-2-ylmethyl)- 1,2,3,4-tetrahydroquinolin-6-yl)urea |
| 3 | 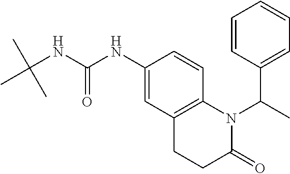 | 1-(tert-butyl)-3-(2-oxo-1-(1-phenylethyl)-1,2,3,4- tetrahydroquinolin-6-yl)urea |
| 4 | 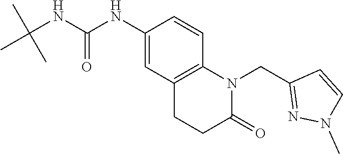 [SMILES: Cn1ccc(CN2C(=O)CCc3cc(NC(=O)NC(C)(C)C)ccc32)n1] | 1-(tert-butyl)-3-(1-((1-methyl-1H-pyrazol-3- yl)methyl)-2-oxo-1,2,3,4-tetrahydroquinolin-6- yl)urea |
| 5 | 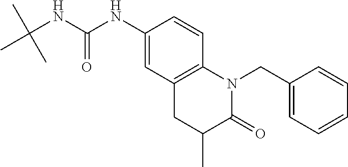 [SMILES: CC1Cc2cc(NC(=O)NC(C)(C)C)ccc2N(Cc2ccccc2)C1=O] | 1-(1-benzyl-3-methyl-2-oxo-1,2,3,4- tetrahydroquinolin-6-yl)-3-(tert-butyl)urea |
| 6 | 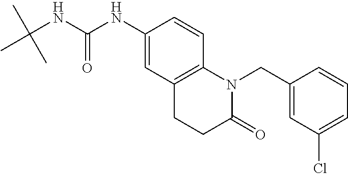 | 1-(tert-butyl)-3-(1-(3-chlorobenzyl)-2-oxo- 1,2,3,4-tetrahydroquinolin-6-yl)urea |
| 7 | 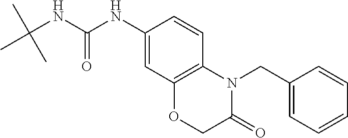 [SMILES: CC(C)(C)NC(=O)Nc1ccc2c(c1)OCC(=O)N2Cc1ccccc1] | 1-(4-benzyl-3-oxo-3,4-dihydro-2H- benzo[b][1,4]oxazin-7-yl)-3-(tert-butyl)urea |
| 8 | 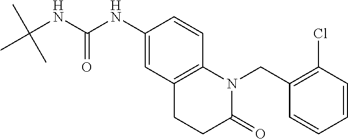 [SMILES: CC(C)(C)NC(=O)Nc1ccc2c(c1)CCC(=O)N2Cc1ccccc1Cl] | 1-(tert-butyl)-3-(1-(2-chlorobenzyl)-2-oxo- 1,2,3,4-tetrahydroquinolin-6-yl)urea |
| 9 | 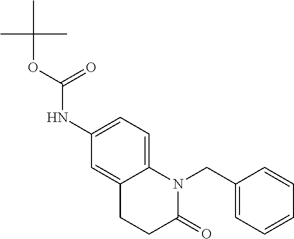 [SMILES: CC(C)(C)OC(=O)Nc1ccc2c(c1)CCC(=O)N2Cc1ccccc1] | tert-butyl (1-benzyl-2-oxo-1,2,3,4- tetrahydroquinolin-6-yl)carbamate |
| 10 | 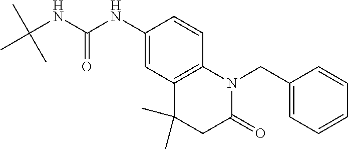 [SMILES: CC(C)(C)NC(=O)Nc1ccc2c(c1)C(C)(C)CC(=O)N2Cc1ccccc1] | 1-(1-benzyl-4,4-dimethyl-2-oxo-1,2,3,4- tetrahydroquinolin-6-yl)-3-(tert-butyl)urea |
| 11 | 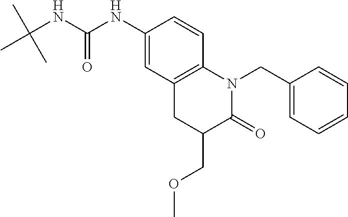 [SMILES: COCC1Cc2cc(NC(=O)NC(C)(C)C)ccc2N(Cc2ccccc2)C1=O] | 1-(1-benzyl-3-(methoxymethyl)-2-oxo-1,2,3,4- tetrahydroquinolin-6-yl)-3-(tert-butyl)urea |
| 12 | 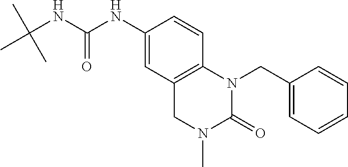 [SMILES: CN1Cc2cc(NC(=O)NC(C)(C)C)ccc2N(Cc2ccccc2)C1=O] | 1-(1-benzyl-3-methyl-2-oxo-1,2,3,4- tetrahydroquinazolin-6-yl)-3-(tert-butyl)urea |
| 13 | 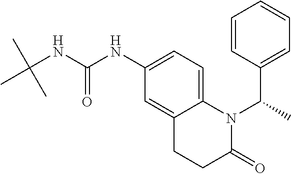 | (S)-1-(tert-butyl)-3-(2-oxo-1-(1-phenylethyl)- 1,2,3,4-tetrahydroquinolin-6-yl)urea |
| 14 | 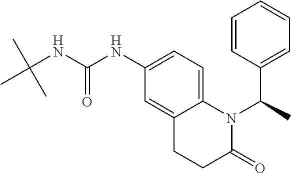 | (R)-1-(tert-butyl)-3-(2-oxo-1-(1-phenylethyl)- 1,2,3,4-tetrahydroquinolin-6-yl)urea |
| 15 | 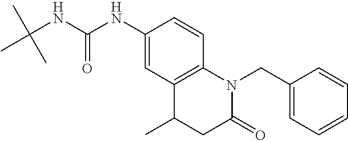 | 1-(1-benzyl-4-methyl-2-oxo-1,2,3,4- tetrahydroquinolin-6-yl)-3-(tert-butyl)urea |
| 16 | 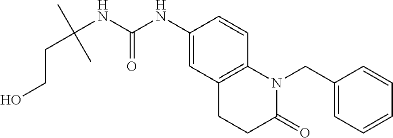 [SMILES: CC(C)(CCO)NC(=O)Nc1ccc2c(c1)CCC(=O)N2Cc1ccccc1] | 1-(1-benzyl-2-oxo-1,2,3,4-tetrahydroquinolin-6- yl)-3-(4-hydroxy-2-methylbutan-2-yl)urea |
| 17 | 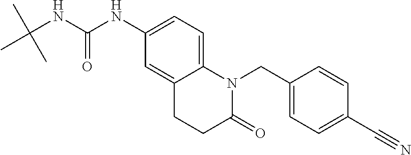 [SMILES: CC(C)(C)NC(=O)Nc1ccc2c(c1)CCC(=O)N2Cc1ccc(C#N)cc1] | 1-(tert-butyl)-3-(1-(4-cyanobenzyl)-2-oxo-1,2,3,4- tetrahydroquinolin-6-yl)urea |
| 18 | 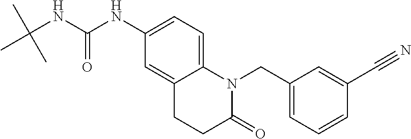 [SMILES: CC(C)(C)NC(=O)Nc1ccc2c(c1)CCC(=O)N2Cc1cccc(C#N)c1] | 1-(tert-butyl)-3-(1-(3-cyanobenzyl)-2-oxo-1,2,3,4- tetrahydroquinolin-6-yl)urea |
| 19 | 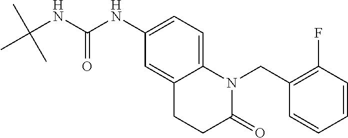 [SMILES: CC(C)(C)NC(=O)Nc1ccc2c(c1)CCC(=O)N2Cc1ccccc1F] | 1-(tert-butyl)-3-(1-(2-fluorobenzyl)-2-oxo-1,2,3,4- tetrahydroquinolin-6-yl)urea |
| 20 | 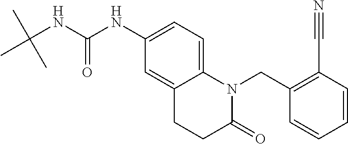 [SMILES: CC(C)(C)NC(=O)Nc1ccc2c(c1)CCC(=O)N2Cc1ccccc1C#N] | 1-(tert-butyl)-3-(1-(2-cyanobenzyl)-2-oxo-1,2,3,4- tetrahydroquinolin-6-yl)urea |
| 21 | 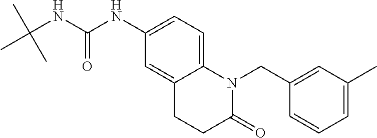 [SMILES: Cc1cccc(CN2C(=O)CCc3cc(NC(=O)NC(C)(C)C)ccc32)c1] | 1-(tert-butyl)-3-(1-(3-methylbenzyl)-2-oxo- 1,2,3,4-tetrahydroquinolin-6-yl)urea |
| 22 | 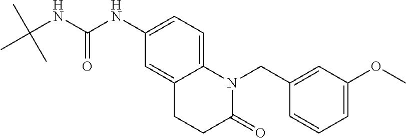 [SMILES: COc1cccc(CN2C(=O)CCc3cc(NC(=O)NC(C)(C)C)ccc32)c1] | 1-(tert-butyl)-3-(1-(3-methoxybenzyl)-2-oxo- 1,2,3,4-tetrahydroquinolin-6-yl)urea |
| 23 | 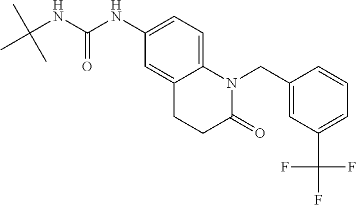 [SMILES: CC(C)(C)NC(=O)Nc1ccc2c(c1)CCC(=O)N2Cc1cccc(C(F)(F)F)c1] | 1-(tert-butyl)-3-(2-oxo-1-(3- (trifluoromethyl)benzyl)-1,2,3,4- tetrahydroquinolin-6-yl)urea |
| 24 | 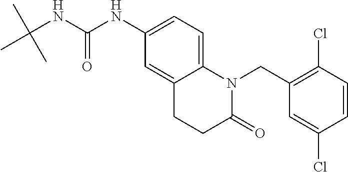 [SMILES: CC(C)(C)NC(=O)Nc1ccc2c(c1)CCC(=O)N2Cc1cc(Cl)ccc1Cl] | 1-(tert-butyl)-3-(1-(2,5-dichlorobenzyl)-2-oxo- 1,2,3,4-tetrahydroquinolin-6-yl)urea |
| 25 | 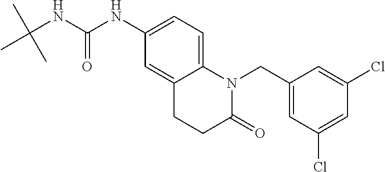 [SMILES: CC(C)(C)NC(=O)Nc1ccc2c(c1)CCC(=O)N2Cc1cc(Cl)cc(Cl)c1] | 1-(tert-butyl)-3-(1-(3,5-dichlorobenzyl)-2-oxo- 1,2,3,4-tetrahydroquinolin-6-yl)urea |
| 26 | 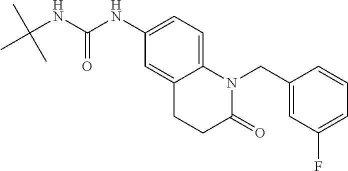 [SMILES: CC(C)(C)NC(=O)Nc1ccc2c(c1)CCC(=O)N2Cc1cccc(F)c1] | 1-(tert-butyl)-3-(1-(3-fluorobenzyl)-2-oxo-1,2,3,4- tetrahydroquinolin-6-yl)urea |
| 27 | 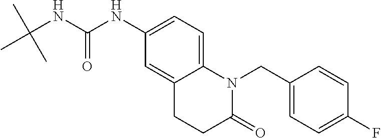 [SMILES: CC(C)(C)NC(=O)Nc1ccc2c(c1)CCC(=O)N2Cc1ccc(F)cc1] | 1-(tert-butyl)-3-(1-(4-fluorobenzyl)-2-oxo-1,2,3,4- tetrahydroquinolin-6-yl)urea |
| 28 | 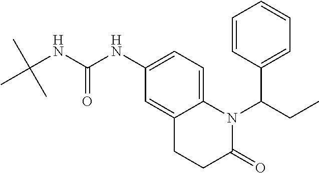 [SMILES: CCC(c1ccccc1)N1C(=O)CCc2cc(NC(=O)NC(C)(C)C)ccc21] | 1-(tert-butyl)-3-(2-oxo-1-(1-phenylpropyl)- 1,2,3,4-tetrahydroquinolin-6-yl)urea |
| 29 | 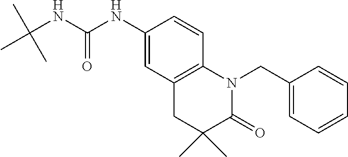 [SMILES: CC(C)(C)NC(=O)Nc1ccc2c(c1)CC(C)(C)C(=O)N2Cc1ccccc1] | 1-(1-benzyl-3,3-dimethyl-2-oxo-1,2,3,4- tetrahydroquinolin-6-yl)-3-(tert-butyl)urea |
| 30 | 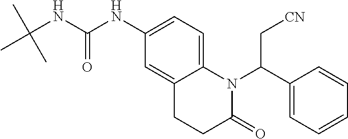 [SMILES: CC(C)(C)NC(=O)Nc1ccc2c(c1)CCC(=O)N2C(CC#N)c1ccccc1] | 1-(tert-butyl)-3-(1-(2-hydroxy-1-phenylethyl)-2- oxo-1,2,3,4-tetrahydroquinolin-6-yl)urea |
| 31 | 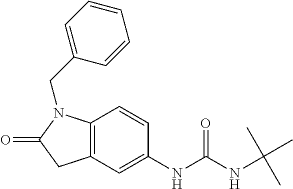 [SMILES: CC(C)(C)NC(=O)Nc1ccc2c(c1)CC(=O)N2Cc1ccccc1] | 1-(1-benzyl-2-oxoindolin-5-yl)-3-(tert-butyl)urea |
| 32 | 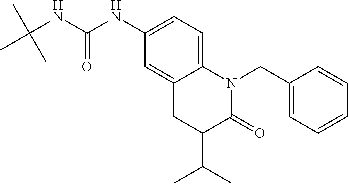 [SMILES: CC(C)C1Cc2cc(NC(=O)NC(C)(C)C)ccc2N(Cc2ccccc2)C1=O] | 1-(1-benzyl-3-isopropyl-2-oxo-1,2,3,4- tetrahydroquinolin-6-yl)-3-(tert-butyl)urea |
| 33 | 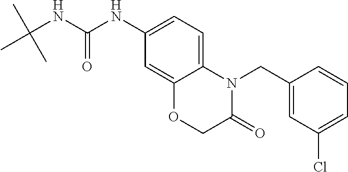 [SMILES: CC(C)(C)NC(=O)Nc1ccc2c(c1)OCC(=O)N2Cc1cccc(Cl)c1] | 1-(tert-butyl)-3-(4-(3-chlorobenzyl)-3-oxo-3,4- dihydro-2H-benzo[b][1,4]oxazin-7-yl)urea |
| 34 | 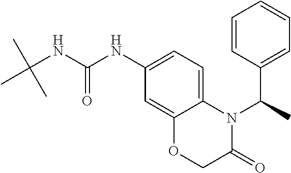 [SMILES: C[C@H](c1ccccc1)N1C(=O)COc2cc(NC(=O)NC(C)(C)C)ccc21] | (R)-1-(tert-butyl)-3-(3-oxo-4-(1-phenylethyl)-3,4- dihydro-2H-benzo[b][1,4]oxazin-7-yl)urea |
| 35 | 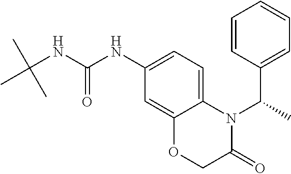 C₂₁H₂₅N₃O₃ | (S)-1-(tert-butyl)-3-(3-oxo-4-(1-phenylethyl)-3,4- dihydro-2H-benzo[b][1,4]oxazin-7-yl)urea |
| 36 | 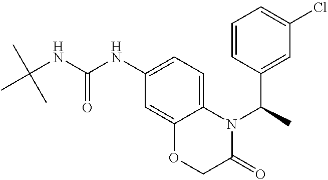 | (R)-1-(tert-butyl)-3-(4-(1-(3-chlorophenyl)ethyl)- 3-oxo-3,4-dihydro-2H-benzo[b][1,4]oxazin-7- yl)urea |
| 37 | 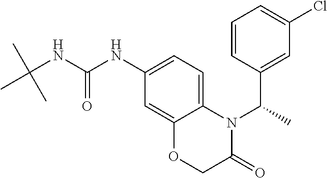 [SMILES: C[C@@H](c1cccc(Cl)c1)N1C(=O)COc2cc(NC(=O)NC(C)(C)C)ccc21] | (S)-1-(tert-butyl)-3-(4-(1-(3-chlorophenyl)ethyl)- 3-oxo-3,4-dihydro-2H-benzo[b][1,4]oxazin-7- yl)urea |
| 38 | 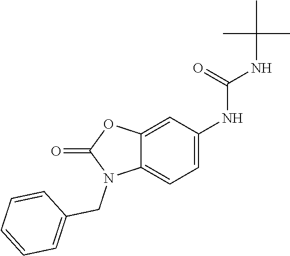 [SMILES: CC(C)(C)NC(=O)Nc1ccc2c(c1)oc(=O)n2Cc1ccccc1] | 1-(3-benzyl-2-oxo-2,3-dihydrobenzo[d]oxazol-6- yl)-3-(tert-butyl)urea |
| 39 | 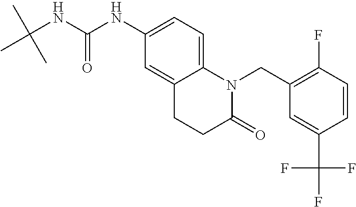 | 1-(tert-butyl)-3-(1-(2-fluoro-5- (trifluoromethyl)benzyl)-2-oxo-1,2,3,4- tetrahydroquinolin-6-yl)urea |
| 40 | 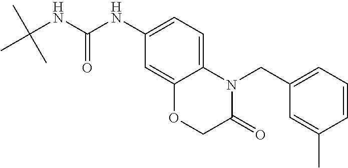 | 1-(tert-butyl)-3-(4-(3-methylbenzyl)-3-oxo-3,4- dihydro-2H-benzo[b][1,4]oxazin-7-yl)urea |
| 41 | 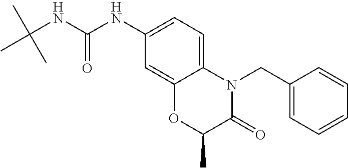 | (R)-1-(4-benzyl-2-methyl-3-oxo-3,4-dihydro-2H- benzo[b][1,4]oxazin-7-yl)-3-(tert-butyl)urea |
| 42 | 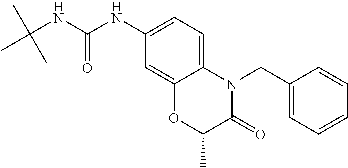 | (S)-1-(4-benzyl-2-methyl-3-oxo-3,4-dihydro-2H- benzo[b][1,4]oxazin-7-yl)-3-(tert-butyl)urea |
| 43 | 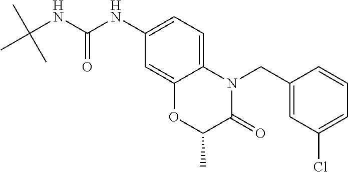 [SMILES: C[C@@H]1Oc2cc(NC(=O)NC(C)(C)C)ccc2N(Cc2cccc(Cl)c2)C1=O] | (S)-1-(tert-butyl)-3-(4-(3-chlorobenzyl)-2-methyl- 3-oxo-3,4-dihydro-2H-benzo[b][1,4]oxazin-7- yl)urea |
| 44 | 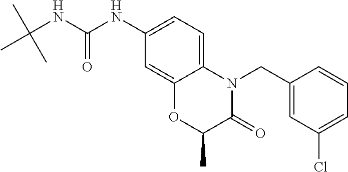 [SMILES: C[C@H]1Oc2cc(NC(=O)NC(C)(C)C)ccc2N(Cc2cccc(Cl)c2)C1=O] | (R)-1-(tert-butyl)-3-(4-(3-chlorobenzyl)-2-methyl- 3-oxo-3,4-dihydro-2H-benzo[b][1,4]oxazin-7- yl)urea |
| 45 | 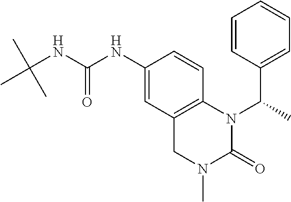 | (S)-1-(tert-butyl)-3-(3-methyl-2-oxo-1-(1- phenylethyl)-1,2,3,4-tetrahydroquinazolin-6- yl)urea |
| 46 | 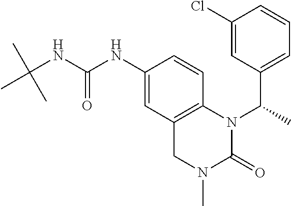 [SMILES: C[C@@H](c1cccc(Cl)c1)N1C(=O)N(C)Cc2cc(NC(=O)NC(C)(C)C)ccc21] | (S)-1-(tert-butyl)-3-(1-(1-(3-chlorophenyl)ethyl)- 3-methyl-2-oxo-1,2,3,4-tetrahydroquinazolin-6- yl)urea |
| 47 | 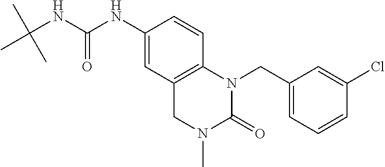 [SMILES: CN1Cc2cc(NC(=O)NC(C)(C)C)ccc2N(Cc2cccc(Cl)c2)C1=O] | 1-(tert-butyl)-3-(1-(3-chlorobenzyl)-3-methyl-2- oxo-1,2,3,4-tetrahydroquinazolin-6-yl)urea |
| 48 | 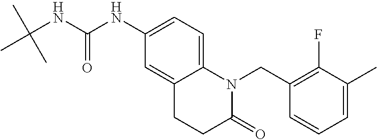 | 1-(tert-butyl)-3-(1-(2-fluoro-3-methylbenzyl)-2- oxo-1,2,3,4-tetrahydroquinolin-6-yl)urea |
| 49 | 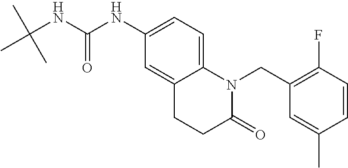 | 1-(tert-butyl)-3-(1-(2-fluoro-5-methylbenzyl)-2- oxo-1,2,3,4-tetrahydroquinolin-6-yl)urea |
| 50 | 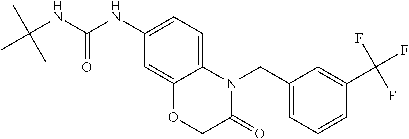 [SMILES: CC(C)(C)NC(=O)Nc1ccc2c(c1)OCC(=O)N2Cc1cccc(C(F)(F)F)c1] | 1-(tert-butyl)-3-(3-oxo-4-(3- (trifluoromethyl)benzyl)-3,4-dihydro-2H- benzo[b][1,4]oxazin-7-yl)urea |
| 51 | 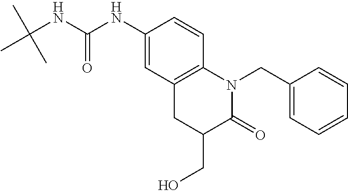 | 1-(1-benzyl-3-(hydroxymethyl)-2-oxo-1,2,3,4- tetrahydroquinolin-6-yl)-3-(tert-butyl)urea |
| 52 | 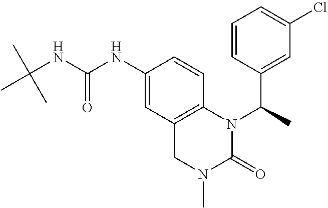 | (R)-1-(tert-butyl)-3-(1-(1-(3-chlorophenyl)ethyl)- 3-methyl-2-oxo-1,2,3,4-tetrahydroquinazolin-6- yl)urea |
| 53 | 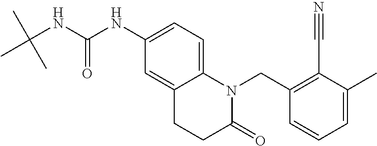 | 1-(tert-butyl)-3-(1-(2-cyano-3-methylbenzyl)-2- oxo-1,2,3,4-tetrahydroquinolin-6-yl)urea |
| 54 | 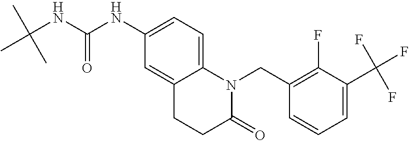 | 1-(tert-butyl)-3-(1-(2-fluoro-3- (trifluoromethyl)benzyl)-2-oxo-1,2,3,4- tetrahydroquinolin-6-yl)urea |
| 55 | 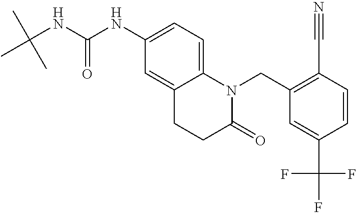 | 1-(tert-butyl)-3-(1-(2-cyano-5- (trifluoromethyl)benzyl)-2-oxo-1,2,3,4- tetrahydroquinolin-6-yl)urea |
| 56 | 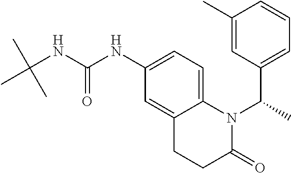 [SMILES: Cc1cccc([C@H](C)N2C(=O)CCc3cc(NC(=O)NC(C)(C)C)ccc32)c1] | (S)-1-(tert-butyl)-3-(2-oxo-1-(1-(m-tolyl)ethyl)- 1,2,3,4-tetrahydroquinolin-6-yl)urea |
| 57 | 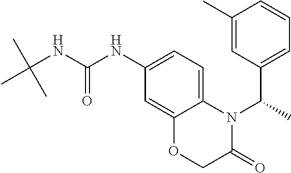 [SMILES: Cc1cccc([C@H](C)N2C(=O)COc3cc(NC(=O)NC(C)(C)C)ccc32)c1] | (S)-1-(tert-butyl)-3-(3-oxo-4-(1-(m-tolyl)ethyl)- 3,4-dihydro-2H-benzo[b][1,4]oxazin-7-yl)urea |
| 58 | 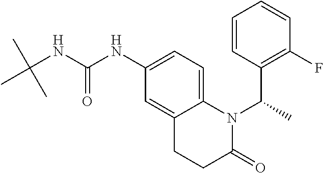 [SMILES: C[C@@H](c1ccccc1F)N1C(=O)CCc2cc(NC(=O)NC(C)(C)C)ccc21] | (S)-1-(tert-butyl)-3-(1-(1-(2-fluorophenyl)ethyl)- 2-oxo-1,2,3,4-tetrahydroquinolin-6-yl)urea |
| 59 | 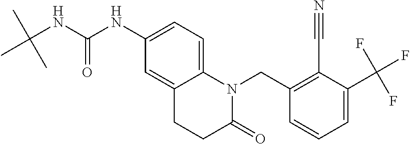 [SMILES: CC(C)(C)NC(=O)Nc1ccc2c(c1)CCC(=O)N2Cc1cccc(C(F)(F)F)c1C#N] | 1-(tert-butyl)-3-(1-(2-cyano-3- (trifluoromethyl)benzyl)-2-oxo-1,2,3,4- tetrahydroquinolin-6-yl)urea |
| 60 | 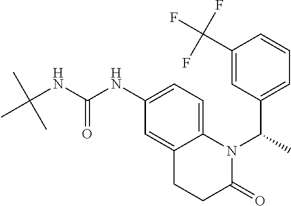 [SMILES: C[C@@H](c1cccc(C(F)(F)F)c1)N1C(=O)CCc2cc(NC(=O)NC(C)(C)C)ccc21] | (S)-1-(tert-butyl)-3-(2-oxo-1-(1-(3- (trifluoromethyl)phenyl)ethyl)-1,2,3,4- tetrahydroquinolin-6-yl)urea |
| 61 | 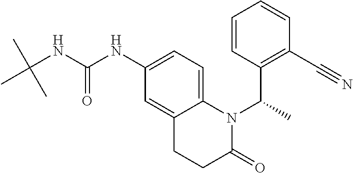 [SMILES: C[C@@H](c1ccccc1C#N)N1C(=O)CCc2cc(NC(=O)NC(C)(C)C)ccc21] | (S)-1-(tert-butyl)-3-(1-(1-(2-cyanophenyl)ethyl)- 2-oxo-1,2,3,4-tetrahydroquinolin-6-yl)urea |
| 62 | 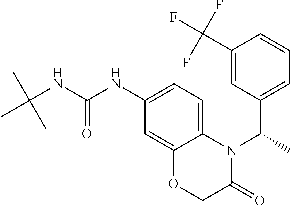 [SMILES: C[C@@H](c1cccc(C(F)(F)F)c1)N1C(=O)COc2cc(NC(=O)NC(C)(C)C)ccc21] | (S)-1-(tert-butyl)-3-(3-oxo-4-(1-(3- (trifluoromethyl)phenyl)ethyl)-3,4-dihydro-2H- benzo[b][1,4]oxazin-7-yl)urea |
| 63 | 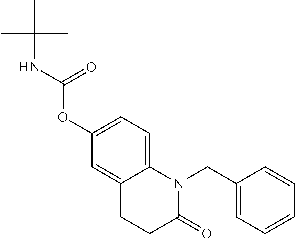 [SMILES: CC(C)(C)NC(=O)Oc1ccc2c(c1)CCC(=O)N2Cc1ccccc1] | 1-benzyl-2-oxo-1,2,3,4-tetrahydroquinolin-6-yl tert-butylcarbamate |
| 64 | 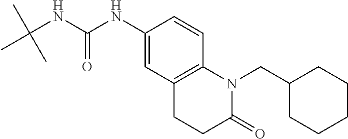 [SMILES: CC(C)(C)NC(=O)Nc1ccc2c(c1)CCC(=O)N2CC1CCCCC1] | 1-(tert-butyl)-3-(1-(cyclohexylmethyl)-2-oxo- 1,2,3,4-tetrahydroquinolin-6-yl)urea |
| 65 | 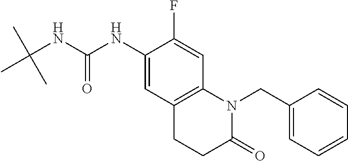 [SMILES: CC(C)(C)NC(=O)Nc1cc2c(cc1F)N(Cc1ccccc1)C(=O)CC2] | 1-(1-benzyl-7-fluoro-2-oxo-1,2,3,4- tetrahydroquinolin-6-yl)-3-(tert-butyl)urea |
| 66 | 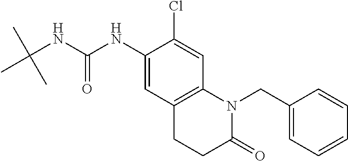 [SMILES: CC(C)(C)NC(=O)Nc1cc2c(cc1Cl)N(Cc1ccccc1)C(=O)CC2] | 1-(1-benzyl-7-chloro-2-oxo-1,2,3,4- tetrahydroquinolin-6-yl)-3-(tert-butyl)urea |
| 67 | 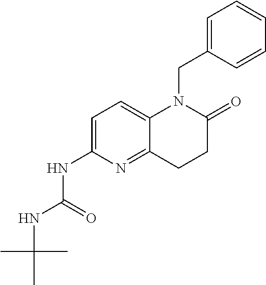 [SMILES: CC(C)(C)NC(=O)Nc1ccc2c(n1)CCC(=O)N2Cc1ccccc1] | 1-(5-benzyl-6-oxo-5,6,7,8-tetrahydro-1,5- naphthyridin-2-yl)-3-(tert-butyl)urea |
| 68 | 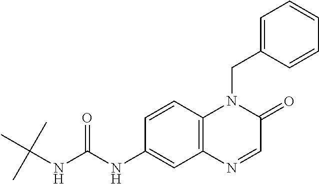 [SMILES: CC(C)(C)NC(=O)Nc1ccc2c(c1)ncc(=O)n2Cc1ccccc1] | 1-(1-benzyl-2-oxo-1,2-dihydroquinoxalin-6-yl)-3- (tert-butyl)urea |
| 69 | 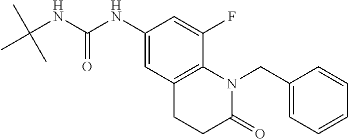 [SMILES: CC(C)(C)NC(=O)Nc1cc(F)c2c(c1)CCC(=O)N2Cc1ccccc1] | 1-(1-benzyl-8-fluoro-2-oxo-1,2,3,4- tetrahydroquinolin-6-yl)-3-(tert-butyl)urea; and |
| 70 | 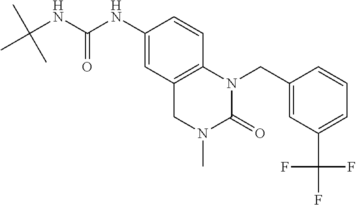 [SMILES: CN1Cc2cc(NC(=O)NC(C)(C)C)ccc2N(Cc2cccc(C(F)(F)F)c2)C1=O] | 1-(tert-butyl)-3-(3-methyl-2-oxo-1-(3- (trifluoromethyl)benzyl)-1,2,3,4- tetrahydroquinazolin-6-yl)urea |
| 71 | 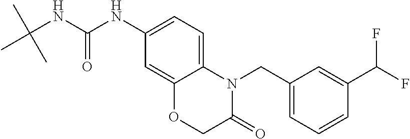 [SMILES: CC(C)(C)NC(=O)Nc1ccc2c(c1)OCC(=O)N2Cc1cccc(C(F)F)c1] C₂₁H₂₃F₂N₃O₃ | 1-(tert-butyl)-3-(4-(3-(difluoromethyl)benzyl)-3- oxo-3,4-dihydro-2H-benzo[b][1,4]oxazin-7- yl)urea |
| 72 | 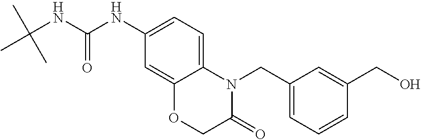 C₂₁H₂₅N₃O₄ | 1-(tert-butyl)-3-(4-(3-(hydroxymethyl)benzyl)-3- oxo-3,4-dihydro-2H-benzo[b][1,4]oxazin-7- yl)urea |
| 73 | 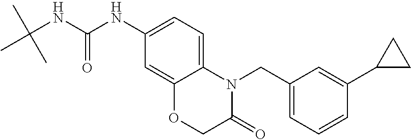 C₂₃H₂₇N₃O₃ | 1-(tert-butyl)-3-(4-(3-cyclopropylbenzyl)-3-oxo- 3,4-dihydro-2H-benzo[b][1,4]oxazin-7-yl)urea |
| 74 | 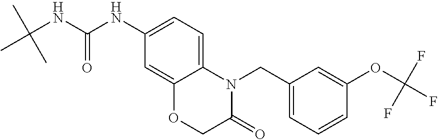 C₂₁H₂₂F₃N₃O₄ | 1-(tert-butyl)-3-(3-oxo-4-(3- (trifluoromethoxy)benzyl)-3,4-dihydro-2H- benzo[b][1,4]oxazin-7-yl)urea |
| 75 | 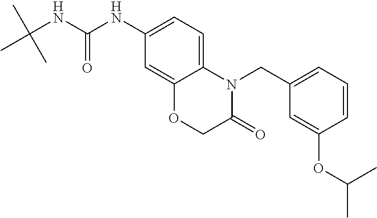 C₂₃H₂₉N₃O₄ | 1-(tert-butyl)-3-(4-(3-isopropoxybenzyl)-3-oxo- 3,4-dihydro-2H-benzo[b][1,4]oxazin-7-yl)urea |
| 76 | 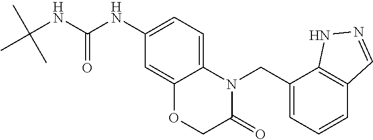 C₂₁H₂₃N₅O₃ | 1-(4-((1H-indazol-7-yl)methyl)-3-oxo-3,4- dihydro-2H-benzo[b][1,4]oxazin-7-yl)-3-(tert- butyl)urea |
| 77 | 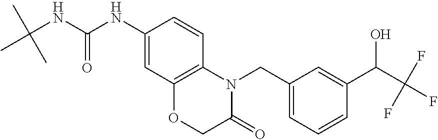 C₂₂H₂₄F₃N₃O₄ | 1-(tert-butyl)-3-(3-oxo-4-(3-(2,2,2-trifluoro-1- hydroxyethyl)benzyl)-3,4-dihydro-2H- benzo[b][1,4]oxazin-7-yl)urea |
| 78 | 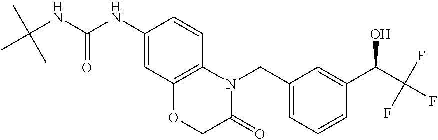 C₂₂H₂₄F₃N₃O₄ | (R)-1-(tert-butyl)-3-(3-oxo-4-(3-(2,2,2-trifluoro-1- hydroxyethyl)benzyl)-3,4-dihydro-2H- benzo[b][1,4]oxazin-7-yl)urea |
| 79 | 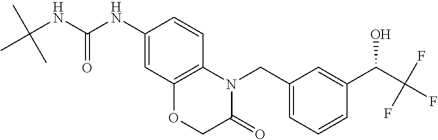 C₂₂H₂₄F₃N₃O₄ | (S)-1-(tert-butyl)-3-(3-oxo-4-(3-(2,2,2-trifluoro-1- hydroxyethyl)benzyl)-3,4-dihydro-2H- benzo[b][1,4]oxazin-7-yl)urea |
| 80 | 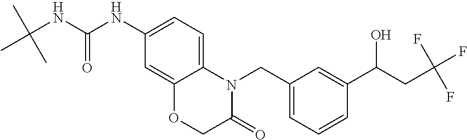 C₂₃H₂₆F₃N₃O₄ | 1-(tert-butyl)-3-(3-oxo-4-(3-(3,3,3-trifluoro-1- hydroxypropyl)benzyl)-3,4-dihydro-2H- benzo[b][1,4]oxazin-7-yl)urea |
| 81 | 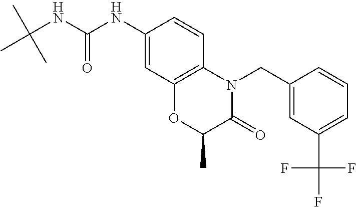 C₂₂H₂₄F₃N₃O₃ | (R)-1-(tert-butyl)-3-(2-methyl-3-oxo-4-(3- (trifluoromethyl)benzyl)-3,4-dihydro-2H- benzo[b][1,4]oxazin-7-yl)urea |
| 82 | 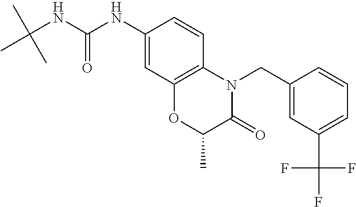 C₂₂H₂₄F₃N₃O₃ | (S)-1-(tert-butyl)-3-(2-methyl-3-oxo-4-(3- (trifluoromethyl)benzyl)-3,4-dihydro-2H- benzo[b][1,4]oxazin-7-yl)urea |
| 83 | 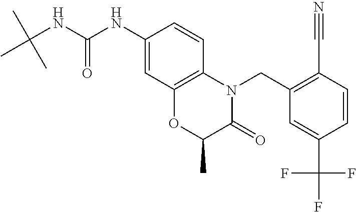 C₂₃H₂₃F₃N₄O₃ | (R)-1-(tert-butyl)-3-(4-(2-cyano-5- (trifluoromethyl)benzyl)-2-methyl-3-oxo-3,4- dihydro-2H-benzo[b][1,4]oxazin-7-yl)urea |
| 84 | 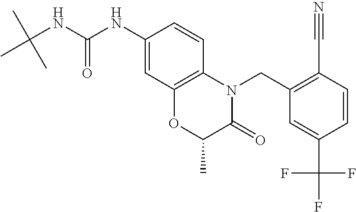 C₂₃H₂₃F₃N₄O₃ | (S)-1-(tert-butyl)-3-(4-(2-cyano-5- (trifluoromethyl)benzyl)-2-methyl-3-oxo-3,4- dihydro-2H-benzo[b][1,4]oxazin-7-yl)urea |
| 85 | 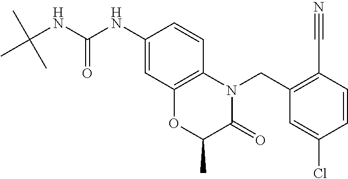 C₂₂H₂₃ClN₄O₃ | (R)-1-(tert-butyl)-3-(4-(5-chloro-2-cyanobenzyl)- 2-methyl-3-oxo-3,4-dihydro-2H- benzo[b][1,4]oxazin-7-yl)urea |
| 86 | 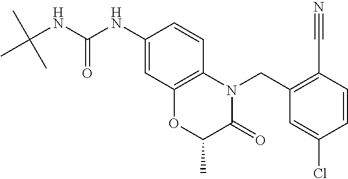 C₂₂H₂₃ClN₄O₃ | (S)-1-(tert-butyl)-3-(4-(5-chloro-2-cyanobenzyl)- 2-methyl-3-oxo-3,4-dihydro-2H- benzo[b][1,4]oxazin-7-yl)urea |
| 87 | 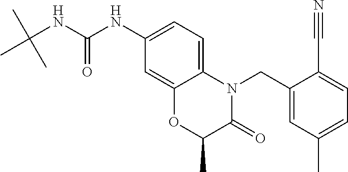 [SMILES: Cc1ccc(C#N)c(CN2C(=O)[C@@H](C)Oc3cc(NC(=O)NC(C)(C)C)ccc32)c1] C₂₃H₂₆N₄O₃ | (R)-1-(tert-butyl)-3-(4-(2-cyano-5-methylbenzyl)- 2-methyl-3-oxo-3,4-dihydro-2H- benzo[b][1,4]oxazin-7-yl)urea |
| 88 | 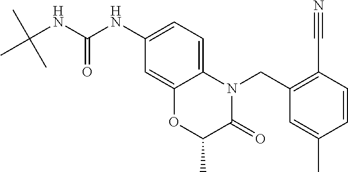 [SMILES: Cc1ccc(C#N)c(CN2C(=O)[C@H](C)Oc3cc(NC(=O)NC(C)(C)C)ccc32)c1] C₂₃H₂₆N₄O₃ | (S)-1-(tert-butyl)-3-(4-(2-cyano-5-methylbenzyl)- 2-methyl-3-oxo-3,4-dihydro-2H- benzo[b][1,4]oxazin-7-yl)urea |
| 89 | 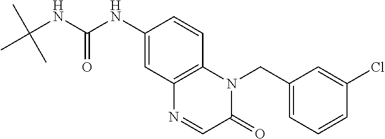 [SMILES: CC(C)(C)NC(=O)Nc1ccc2c(c1)ncc(=O)n2Cc1cccc(Cl)c1] C₂₀H₂₁ClN₄O₂ | 1-(tert-butyl)-3-(1-(3-chlorobenzyl)-2-oxo-1,2- dihydroquinoxalin-6-yl)urea |
| 90 | 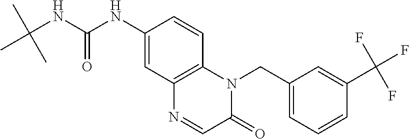 [SMILES: CC(C)(C)NC(=O)Nc1ccc2c(c1)ncc(=O)n2Cc1cccc(C(F)(F)F)c1] C₂₁H₂₁F₃N₄O₂ | 1-(tert-butyl)-3-(2-oxo-1-(3- (trifluoromethyl)benzyl)-1,2-dihydroquinoxalin-6- yl)urea |
| 91 | 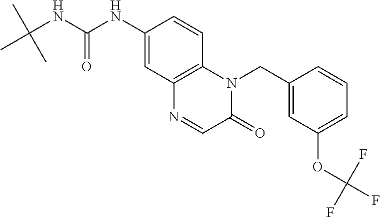 [SMILES: CC(C)(C)NC(=O)Nc1ccc2c(c1)ncc(=O)n2Cc1cccc(OC(F)(F)F)c1] C₂₁H₂₁F₃N₄O₃ | 1-(tert-butyl)-3-(2-oxo-1-(3- (trifluoromethoxy)benzyl)-1,2-dihydroquinoxalin- 6-yl)urea |
| 92 | 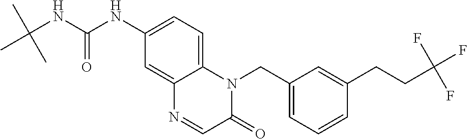 [SMILES: CC(C)(C)NC(=O)Nc1ccc2c(c1)ncc(=O)n2Cc1cccc(CCC(F)(F)F)c1] C₂₃H₂₅F₃N₄O₂ | 1-(tert-butyl)-3-(2-oxo-1-(3-(3,3,3- trifluoropropyl)benzyl)-1,2-dihydroquinoxalin-6- yl)urea |
| 93 | 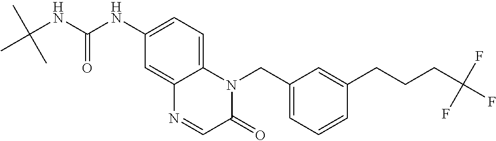 C₂₄H₂₇F₃N₄O₂ | 1-(tert-butyl)-3-(2-oxo-1-(3-(4,4,4- trifluorobutyl)benzyl)-1,2-dihydroquinoxalin-6- yl)urea |
| 94 | 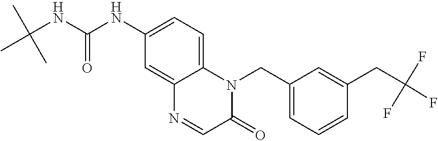 C₂₂H₂₃F₃N₄O₂ | 1-(tert-butyl)-3-(2-oxo-1-(3-(2,2,2- trifluoroethyl)benzyl)-1,2-dihydroquinoxalin-6- yl)urea |
| 95 | 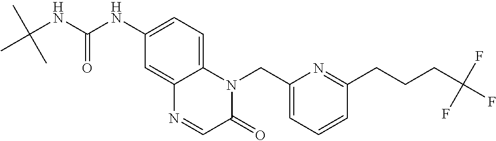 C₂₃H₂₆F₃N₅O₂ | 1-(tert-butyl)-3-(2-oxo-1-((6-(4,4,4- trifluorobutyl)pyridin-2-yl)methyl)-1,2- dihydroquinoxalin-6-yl)urea |
| 96 | 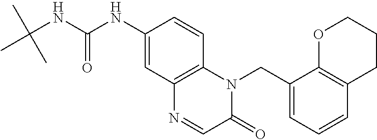 C₂₃H₂₆N₄O₃ | 1-(tert-butyl)-3-(1-(chroman-8-ylmethyl)-2-oxo- 1,2-dihydroquinoxalin-6-yl)urea |
| 97 | 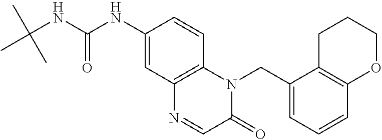 C₂₃H₂₆N₄O₃ | 1-(tert-butyl)-3-(1-(chroman-5-ylmethyl)-2-oxo- 1,2-dihydroquinoxalin-6-yl)urea |
| 98 | 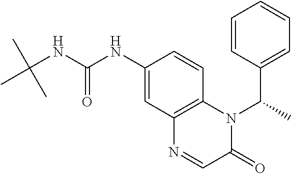 C₂₁H₂₄N₄O₂ | (S)-1-(tert-butyl)-3-(2-oxo-1-(1-phenylethyl)-1,2- dihydroquinoxalin-6-yl)urea |
| 99 | 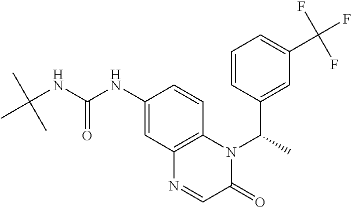 C₂₂H₂₃F₃N₄O₂ | (S)-1-(tert-butyl)-3-(2-oxo-1-(1-(3- (trifluoromethyl)phenyl)ethyl)-1,2- dihydroquinoxalin-6-yl)urea |
| 100 | 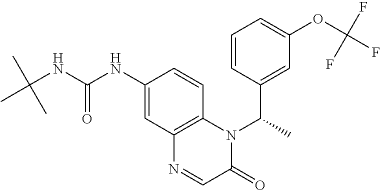 C₂₂H₂₃F₃N₄O₃ | (S)-1-(tert-butyl)-3-(2-oxo-1-(1-(3- (trifluoromethoxy)phenyl)ethyl)-1,2- dihydroquinoxalin-6-yl)urea |
| 101 | 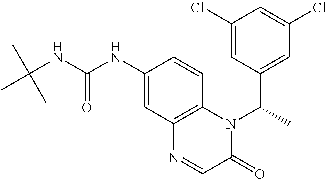 C₂₁H₂₂Cl₂N₄O₂ | (S)-1-(tert-butyl)-3-(1-(1-(3,5- dichlorophenyl)ethyl)-2-oxo-1,2- dihydroquinoxalin-6-yl)urea |
| 102 | 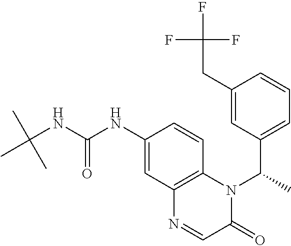 C₂₃H₂₅F₃N₄O₂ | (S)-1-(tert-butyl)-3-(2-oxo-1-(1-(3-(2,2,2- trifluoroethyl)phenyl)ethyl)-1,2- dihydroquinoxalin-6-yl)urea |
| 103 | 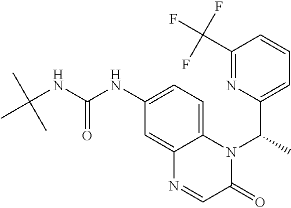 C₂₁H₂₂F₃N₅O₂ | (S)-1-(tert-butyl)-3-(2-oxo-1-(1-(6- (trifluoromethyl)pyridin-2-yl)ethyl)-1,2- dihydroquinoxalin-6-yl)urea |
| 104 | 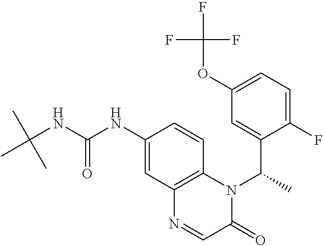 C₂₂H₂₂F₄N₄O₃ | (S)-1-(tert-butyl)-3-(1-(1-(2-fluoro-5- (trifluoromethoxy)phenyl)ethyl)-2-oxo-1,2- dihydroquinoxalin-6-yl)urea |
| 105 | 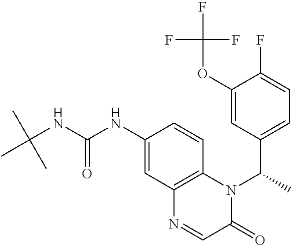 C₂₂H₂₂F₄N₄O₃ | (S)-1-(tert-butyl)-3-(1-(1-(4-fluoro-3- (trifluoromethoxy)phenyl)ethyl)-2-oxo-1,2- dihydroquinoxalin-6-yl)urea |
| 106 | 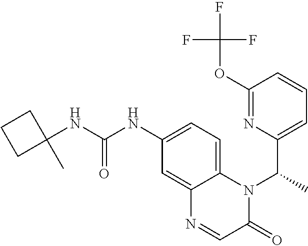 C₂₂H₂₂F₃N₅O₃ | (S)-1-(1-methylcyclobutyl)-3-(2-oxo-1-(1-(6- (trifluoromethoxy)pyridin-2-yl)ethyl)-1,2- dihydroquinoxalin-6-yl)urea |
| 107 | 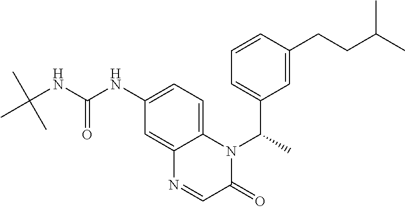 C₂₆H₃₄N₄O₂ | (S)-1-(tert-butyl)-3-(1-(1-(3- isopentylphenyl)ethyl)-2-oxo-1,2- dihydroquinoxalin-6-yl)urea |
| 108 | 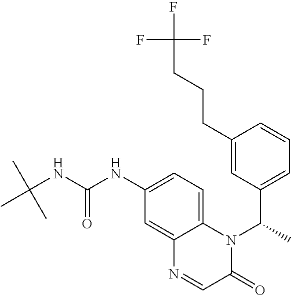 C₂₅H₂₉F₃N₄O₂ | (S)-1-(tert-butyl)-3-(2-oxo-1-(1-(3-(4,4,4- trifluorobutyl)phenyl)ethyl)-1,2- dihydroquinoxalin-6-yl)urea |
| 109 | 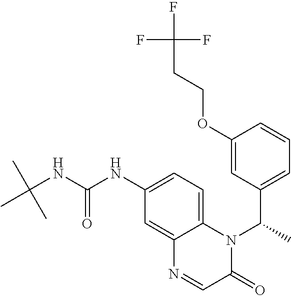 C₂₄H₂₇F₃N₄O₃ | (S)-1-(tert-butyl)-3-(2-oxo-1-(1-(3-(3,3,3- trifluoropropoxy)phenyl)ethyl)-1,2- dihydroquinoxalin-6-yl)urea |
| 110 | 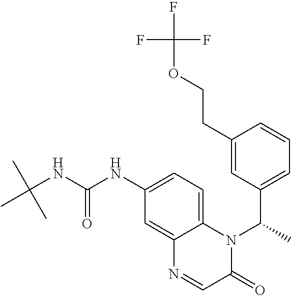 C₂₄H₂₇F₃N₄O₃ | (S)-1-(tert-butyl)-3-(2-oxo-1-(1-(3-(2- (trifluoromethoxy)ethyl)phenyl)ethyl)-1,2- dihydroquinoxalin-6-yl)urea |
| 111 | 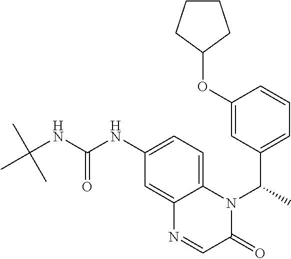 C₂₆H₃₂N₄O₃ | (S)-1-(tert-butyl)-3-(1-(1-(3- (cyclopentyloxy)phenyl)ethyl)-2-oxo-1,2- dihydroquinoxalin-6-yl)urea |
| 112 | 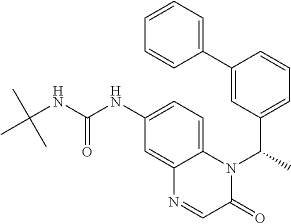 C₂₇H₂₈N₄O₂ | (S)-1-(1-(1-([1,1′-biphenyl]-3-yl)ethyl)-2-oxo-1,2- dihydroquinoxalin-6-yl)-3-(tert-butyl)urea |
| 113 | 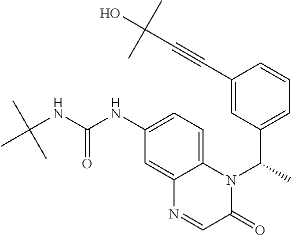 C₂₆H₃₀N₄O₃ | (S)-1-(tert-butyl)-3-(1-(1-(3-(3-hydroxy-3- methylbut-1-yn-1-yl)phenyl)ethyl)-2-oxo-1,2- dihydroquinoxalin-6-yl)urea |
| 114 | 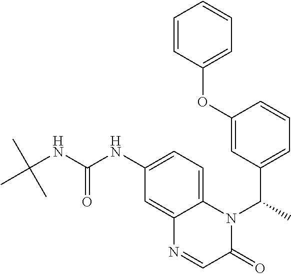 C₂₇H₂₈N₄O₃ | (S)-1-(tert-butyl)-3-(2-oxo-1-(1-(3- phenoxyphenyl)ethyl)-1,2-dihydroquinoxalin-6- yl)urea |
| 115 | 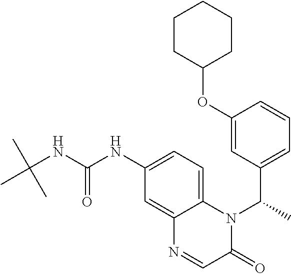 C₂₇H₃₄N₄O₃ | (S)-1-(tert-butyl)-3-(1-(1-(3- (cyclohexyloxy)phenyl)ethyl)-2-oxo-1,2- dihydroquinoxalin-6-yl)urea |
| 116 | 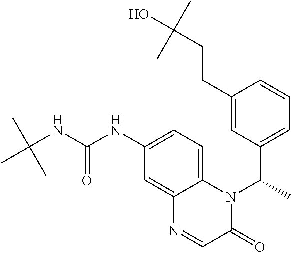 C₂₆H₃₄N₄O₃ | (S)-1-(tert-butyl)-3-(1-(1-(3-(3-hydroxy-3- methylbutyl)phenyl)ethyl)-2-oxo-1,2- dihydroquinoxalin-6-yl)urea |
| 117 | 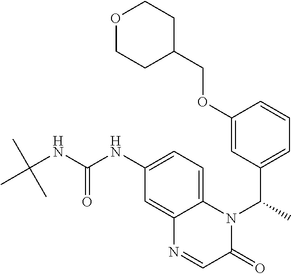 C₂₇H₃₄N₄O₄ | (S)-1-(tert-butyl)-3-(2-oxo-1-(1-(3-((tetrahydro- 2H-pyran-4-yl)methoxy)phenyl)ethyl)-1,2- dihydroquinoxalin-6-yl)urea |
| 118 | 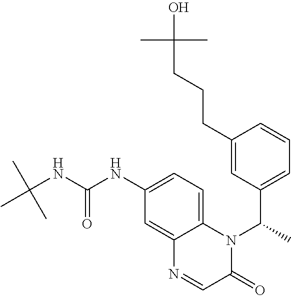 C₂₇H₃₆N₄O₃ | (S)-1-(tert-butyl)-3-(1-(1-(3-(4-hydroxy-4- methylpentyl)phenyl)ethyl)-2-oxo-1,2- dihydroquinoxalin-6-yl)urea |
| 119 | 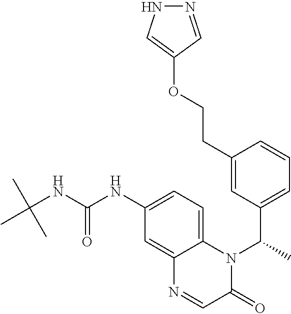 C₂₆H₃₀N₆O₃ | (S)-1-(1-(1-(3-(2-((1H-pyrazol-4- yl)oxy)ethyl)phenyl)ethyl)-2-oxo-1,2- dihydroquinoxalin-6-yl)-3-(tert-butyl)urea |
| 120 | 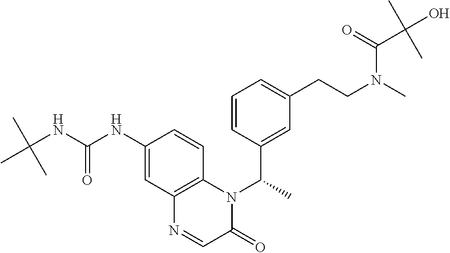 C₂₈H₃₇N₅O₄ | (S)-N-(3-(1-(6-(3-(tert-butyl)ureido)-2- oxoquinoxalin-1(2H)-yl)ethyl)phenethyl)-2- hydroxy-N,2-dimethylpropanamide |
| 121 | 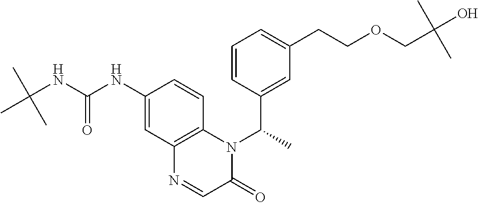 C₂₇H₃₆N₄O₄ | (S)-1-(tert-butyl)-3-(1-(1-(3-(2-(2-hydroxy-2- methylpropoxy)ethyl)phenyl)ethyl)-2-oxo-1,2- dihydroquinoxalin-6-yl)urea |
| 122 | 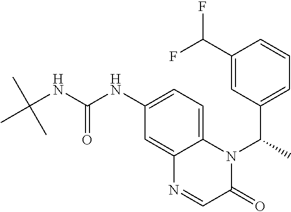 C₂₂H₂₄F₂N₄O₂ | (S)-1-(tert-butyl)-3-(1-(1-(3- (difluoromethyl)phenyl)ethyl)-2-oxo-1,2- dihydroquinoxalin-6-yl)urea |
| 123 | 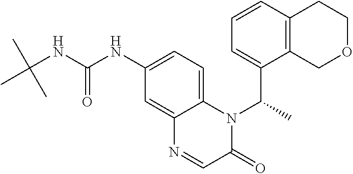 C₂₄H₂₈N₄O₃ | (S)-1-(tert-butyl)-3-(1-(1-(isochroman-8-yl)ethyl)- 2-oxo-1,2-dihydroquinoxalin-6-yl)urea |
| 124 | 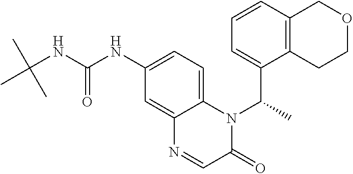 C₂₄H₂₈N₄O₃ | (S)-1-(tert-butyl)-3-(1-(1-(isochroman-5-yl)ethyl)- 2-oxo-1,2-dihydroquinoxalin-6-yl)urea |
| 125 | 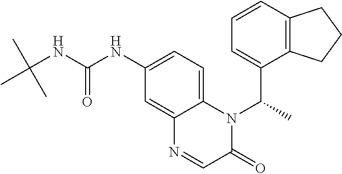 C₂₄H₂₈N₄O₂ | (S)-1-(tert-butyl)-3-(1-(1-(2,3-dihydro-1H-inden- 4-yl)ethyl)-2-oxo-1,2-dihydroquinoxalin-6-yl)urea |
| 126 | 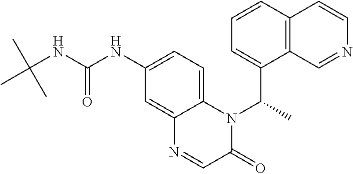 C₂₄H₂₅N₅O₂ | (S)-1-(tert-butyl)-3-(1-(1-(isoquinolin-8-yl)ethyl)- 2-oxo-1,2-dihydroquinoxalin-6-yl)urea |
| 127 | 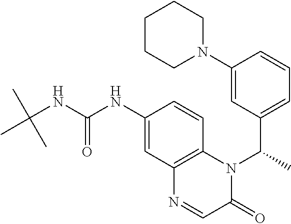 C₂₆H₃₃N₅O₂ | (S)-1-(tert-butyl)-3-(2-oxo-1-(1-(3-(piperidin-1- yl)phenyl)ethyl)-1,2-dihydroquinoxalin-6-yl)urea |
| 128 | 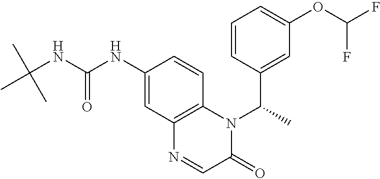 C₂₂H₂₄F₂N₄O₃ | (S)-1-(tert-butyl)-3-(1-(1-(3- (difluoromethoxy)phenyl)ethyl)-2-oxo-1,2- dihydroquinoxalin-6-yl)urea |
| 129 | 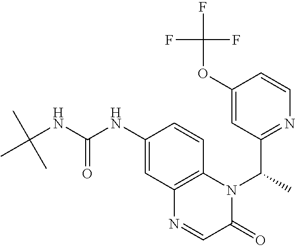 C₂₁H₂₂F₃N₅O₃ | (S)-1-(tert-butyl)-3-(2-oxo-1-(1-(4- (trifluoromethoxy)pyridin-2-yl)ethyl)-1,2- dihydroquinoxalin-6-yl)urea |
| 130 | 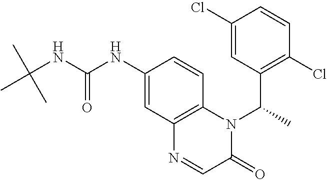 C₂₁H₂₂Cl₂N₄O₂ | (S)-1-(tert-butyl)-3-(1-(1-(2,5- dichlorophenyl)ethyl)-2-oxo-1,2- dihydroquinoxalin-6-yl)urea |
| 131 | 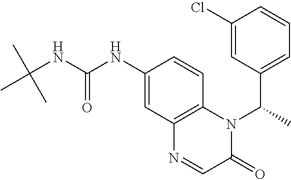 C₂₁H₂₃ClN₄O₂ | (S)-1-(tert-butyl)-3-(1-(1-(3-chlorophenyl)ethyl)- 2-oxo-1,2-dihydroquinoxalin-6-yl)urea |
| 132 | 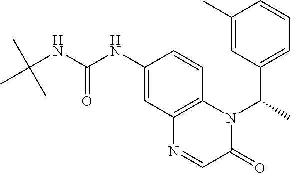 C₂₂H₂₆N₄O₂ | (S)-1-(tert-butyl)-3-(2-oxo-1-(1-(m-tolyl)ethyl)- 1,2-dihydroquinoxalin-6-yl)urea |
| 133 | 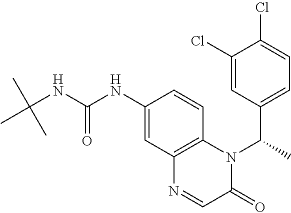 C₂₁H₂₂Cl₂N₄O₂ | (S)-1-(tert-butyl)-3-(1-(1-(3,4- dichlorophenyl)ethyl)-2-oxo-1,2- dihydroquinoxalin-6-yl)urea |
| 134 | 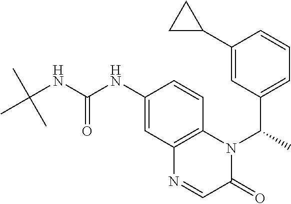 C₂₄H₂₈N₄O₂ | (S)-1-(tert-butyl)-3-(1-(1-(3- cyclopropylphenyl)ethyl)-2-oxo-1,2- dihydroquinoxalin-6-yl)urea |
| 135 | 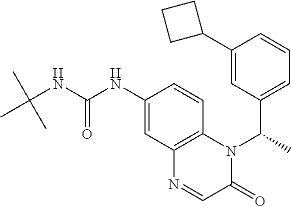 C₂₅H₃₀N₄O₂ | (S)-1-(tert-butyl)-3-(1-(1-(3- cyclobutylphenyl)ethyl)-2-oxo-1,2- dihydroquinoxalin-6-yl)urea |
| 136 | 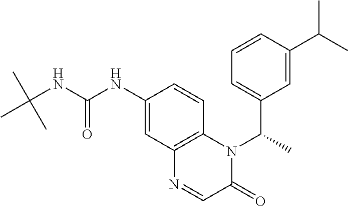 C₂₄H₃₀N₄O₂ | (S)-1-(tert-butyl)-3-(1-(1-(3- isopropylphenyl)ethyl)-2-oxo-1,2- dihydroquinoxalin-6-yl)urea |
| 137 | 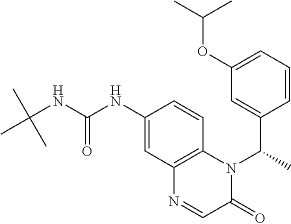 C₂₄H₃₀N₄O₃ | (S)-1-(tert-butyl)-3-(1-(1-(3- isopropoxyphenyl)ethyl)-2-oxo-1,2- dihydroquinoxalin-6-yl)urea |
| 138 | 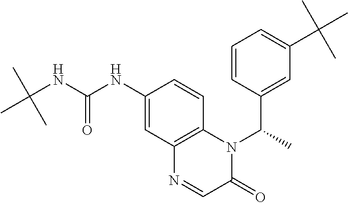 C₂₅H₃₂N₄O₂ | (S)-1-(tert-butyl)-3-(1-(1-(3-(tert- butyl)phenyl)ethyl)-2-oxo-1,2-dihydroquinoxalin- 6-yl)urea |
| 139 | 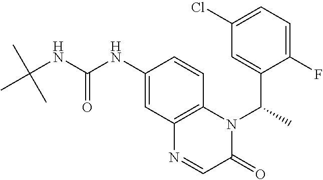 C₂₁H₂₂ClFN₄O₂ | (S)-1-(tert-butyl)-3-(1-(1-(5-chloro-2- fluorophenyl)ethyl)-2-oxo-1,2-dihydroquinoxalin- 6-yl)urea |
| 140 | 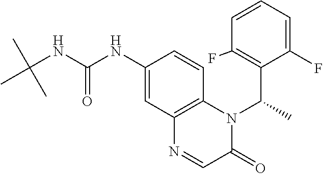 C₂₁H₂₂F₂N₄O₂ | (S)-1-(tert-butyl)-3-(1-(1-(2,6- difluorophenyl)ethyl)-2-oxo-1,2- dihydroquinoxalin-6-yl)urea |
| 141 | 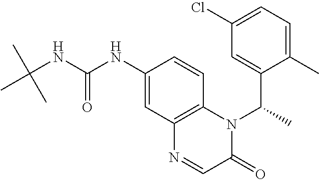 C₂₂H₂₅ClN₄O₂ | (S)-1-(tert-butyl)-3-(1-(1-(5-chloro-2- methylphenyl)ethyl)-2-oxo-1,2- dihydroquinoxalin-6-yl)urea |
| 142 | 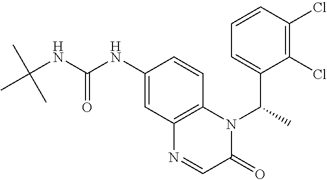 C₂₁H₂₂Cl₂N₄O₂ | (S)-1-(tert-butyl)-3-(1-(1-(2,3- dichlorophenyl)ethyl)-2-oxo-1,2- dihydroquinoxalin-6-yl)urea |
| 143 | 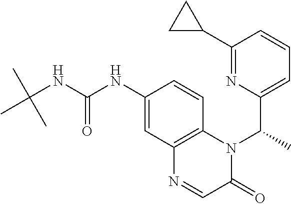 C₂₃H₂₇N₅O₂ | (S)-1-(tert-butyl)-3-(1-(1-(6-cyclopropylpyridin-2- yl)ethyl)-2-oxo-1,2-dihydroquinoxalin-6-yl)urea |
| 144 | 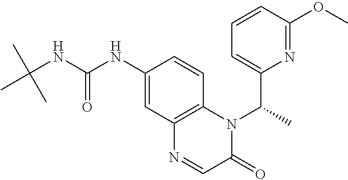 C₂₁H₂₅N₅O₃ | (S)-1-(tert-butyl)-3-(1-(1-(6-methoxypyridin-2- yl)ethyl)-2-oxo-1,2-dihydroquinoxalin-6-yl)urea |
| 145 | 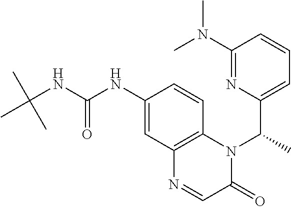 C₂₂H₂₈N₆O₂ | (S)-1-(tert-butyl)-3-(1-(1-(6- (dimethylamino)pyridin-2-yl)ethyl)-2-oxo-1,2- dihydroquinoxalin-6-yl)urea |
| 146 | 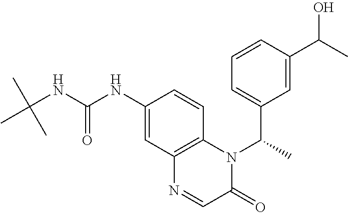 C₂₃H₂₈N₄O₃ | 1-(tert-butyl)-3-(1-((1S)-1-(3-(1- hydroxyethyl)phenyl)ethyl)-2-oxo-1,2- dihydroquinoxalin-6-yl)urea |
| 147 | 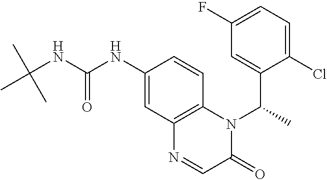 C₂₁H₂₂ClFN₄O₂ | (S)-1-(tert-butyl)-3-(1-(1-(2-chloro-5- fluorophenyl)ethyl)-2-oxo-1,2-dihydroquinoxalin- 6-yl)urea |
| 148 | 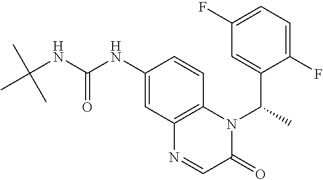 C₂₁H₂₂F₂N₄O₂ | (S)-1-(tert-butyl)-3-(1-(1-(2,5- difluorophenyl)ethyl)-2-oxo-1,2- dihydroquinoxalin-6-yl)urea |
| 149 | 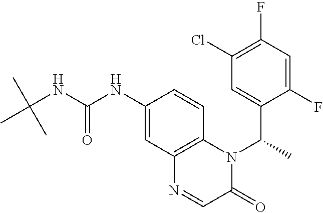 C₂₁H₂₁ClF₂N₄O₂ | (S)-1-(tert-butyl)-3-(1-(1-(5-chloro-2,4- difluorophenyl)ethyl)-2-oxo-1,2- dihydroquinoxalin-6-yl)urea |
| 150 | 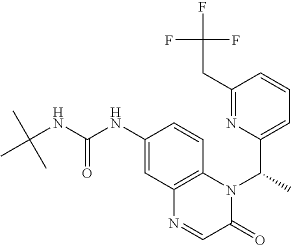 C₂₂H₂₄F₃N₅O₂ | (S)-1-(tert-butyl)-3-(2-oxo-1-(1-(6-(2,2,2- trifluoroethyl)pyridin-2-yl)ethyl)-1,2- dihydroquinoxalin-6-yl)urea |
| 151 | 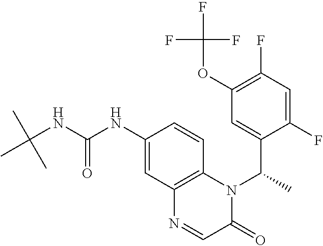 C₂₂H₂₁F₅N₄O₃ | (S)-1-(tert-butyl)-3-(1-(1-(2,4-difluoro-5- (trifluoromethoxy)phenyl)ethyl)-2-oxo-1,2- dihydroquinoxalin-6-yl)urea |
| 152 | 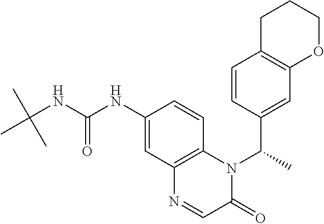 C₂₄H₂₈N₄O₃ | (S)-1-(tert-butyl)-3-(1-(1-(chroman-7-yl)ethyl)-2- oxo-1,2-dihydroquinoxalin-6-yl)urea |
| 153 | 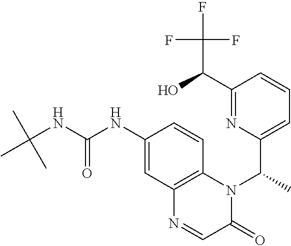 C₂₂H₂₄F₃N₅O₃ | 1-(tert-butyl)-3-(2-oxo-1-((S)-1-(6-((R)-2,2,2- trifluoro-1-hydroxyethyl)pyridin-2-yl)ethyl)-1,2- dihydroquinoxalin-6-yl)urea |
| 154 | 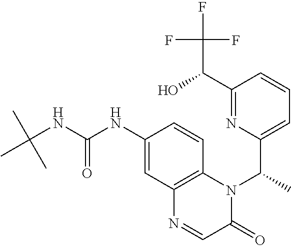 C₂₂H₂₄F₃N₅O₃ | 1-(tert-butyl)-3-(2-oxo-1-((S)-1-(6-((S)-2,2,2- trifluoro-1-hydroxyethyl)pyridin-2-yl)ethyl)-1,2- dihydroquinoxalin-6-yl)urea |
| 155 | 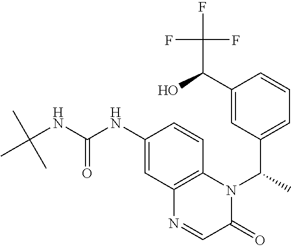 C₂₃H₂₅F₃N₄O₃ | 1-(tert-butyl)-3-(2-oxo-1-((S)-1-(3-((R)-2,2,2- trifluoro-1-hydroxyethyl)phenyl)ethyl)-1,2- dihydroquinoxalin-6-yl)urea |
| 156 | 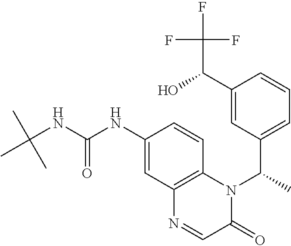 C₂₃H₂₅F₃N₄O₃ | 1-(tert-butyl)-3-(2-oxo-1-((S)-1-(3-((S)-2,2,2- trifluoro-1-hydroxyethyl)phenyl)ethyl)-1,2- dihydroquinoxalin-6-yl)urea |
| 157 | 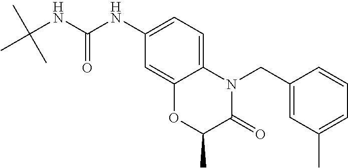 C₂₂H₂₇N₃O₃ | (R)-1-(tert-butyl)-3-(2-methyl-4-(3- methylbenzyl)-3-oxo-3,4-dihydro-2H- benzo[b][1,4]oxazin-7-yl)urea |
| 158 | 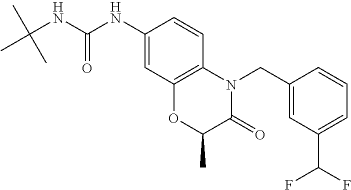 C₂₂H₂₅F₂N₃O₃ | (R)-1-(tert-butyl)-3-(4-(3- (difluoromethyl)benzyl)-2-methyl-3-oxo-3,4- dihydro-2H-benzo[b][1,4]oxazin-7-yl)urea |
| 159 | 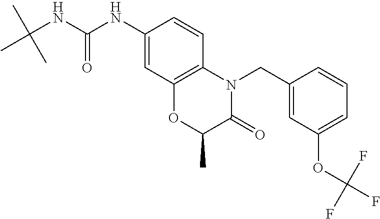 C₂₂H₂₄F₃N₃O₄ | (R)-1-(tert-butyl)-3-(2-methyl-3-oxo-4-(3- (trifluoromethoxy)benzyl)-3,4-dihydro-2H- benzo[b][1,4]oxazin-7-yl)urea |
| 160 | 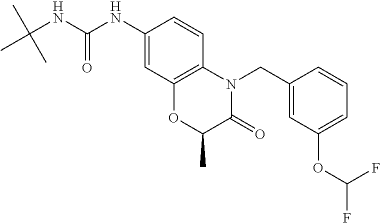 C₂₂H₂₅F₂N₃O₄ | (R)-1-(tert-butyl)-3-(4-(3- (difluoromethoxy)benzyl)-2-methyl-3-oxo-3,4- dihydro-2H-benzo[b][1,4]oxazin-7-yl)urea |
| 161 | 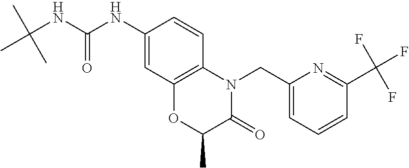 C₂₁H₂₃F₃N₄O₃ | (R)-1-(tert-butyl)-3-(2-methyl-3-oxo-4-((6- (trifluoromethyl)pyridin-2-yl)methyl)-3,4- dihydro-2H-benzo[b][1,4]oxazin-7-yl)urea |
| 162 | 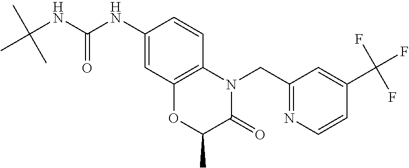 C₂₁H₂₃F₃N₄O₃ | (R)-1-(tert-butyl)-3-(2-methyl-3-oxo-4-((4- (trifluoromethyl)pyridin-2-yl)methyl)-3,4- dihydro-2H-benzo[b][1,4]oxazin-7-yl)urea |
| 163 | 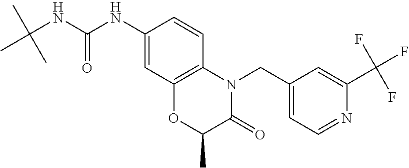 C₂₁H₂₃F₃N₄O₃ | (R)-1-(tert-butyl)-3-(2-methyl-3-oxo-4-((2- (trifluoromethyl)pyridin-4-yl)methyl)-3,4- dihydro-2H-benzo[b][1,4]oxazin-7-yl)urea |
| 164 | 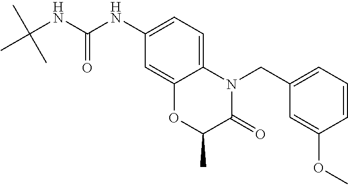 C₂₂H₂₇N₃O₄ | (R)-1-(tert-butyl)-3-(4-(3-methoxybenzyl)-2- methyl-3-oxo-3,4-dihydro-2H- benzo[b][1,4]oxazin-7-yl)urea |
| 165 | 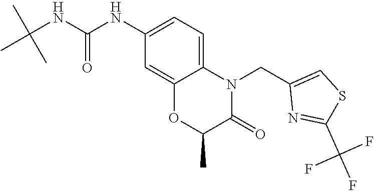 C₁₉H₂₁F₃N₄O₃S | (R)-1-(tert-butyl)-3-(2-methyl-3-oxo-4-((2- (trifluoromethyl)thiazol-4-yl)methyl)-3,4-dihydro- 2H-benzo[b][1,4]oxazin-7-yl)urea |
| 166 | 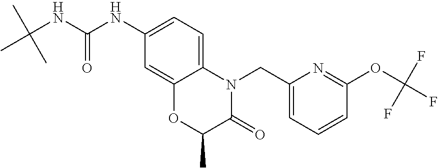 C₂₁H₂₃F₃N₄O₄ | (R)-1-(tert-butyl)-3-(2-methyl-3-oxo-4-((6- (trifluoromethoxy)pyridin-2-yl)methyl)-3,4- dihydro-2H-benzo[b][1,4]oxazin-7-yl)urea |
| 167 | 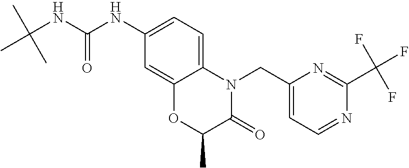 C₂₀H₂₂F₃N₅O₃ | (R)-1-(tert-butyl)-3-(2-methyl-3-oxo-4-((2- (trifluoromethyl)pyrimidin-4-yl)methyl)-3,4- dihydro-2H-benzo[b][1,4]oxazin-7-yl)urea |
| 168 | 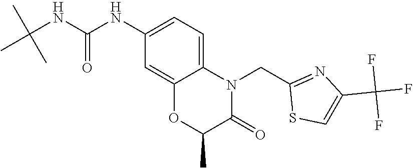 C₁₉H₂₁F₃N₄O₃S | (R)-1-(tert-butyl)-3-(2-methyl-3-oxo-4-((4- (trifluoromethyl)thiazol-2-yl)methyl)-3,4-dihydro- 2H-benzo[b][1,4]oxazin-7-yl)urea |
| 169 | 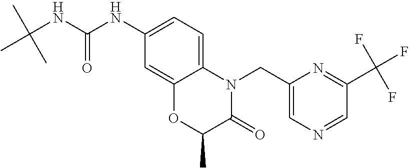 C₂₀H₂₂F₃N₅O₃ | (R)-1-(tert-butyl)-3-(2-methyl-3-oxo-4-((6- (trifluoromethyl)pyrazin-2-yl)methyl)-3,4- dihydro-2H-benzo[b][1,4]oxazin-7-yl)urea |
| 170 | 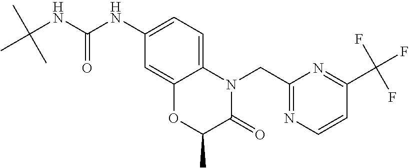 C₂₀H₂₂F₃N₅O₃ | (R)-1-(tert-butyl)-3-(2-methyl-3-oxo-4-((4- (trifluoromethyl)pyrimidin-2-yl)methyl)-3,4- dihydro-2H-benzo[b][1,4]oxazin-7-yl)urea |
| 171 | 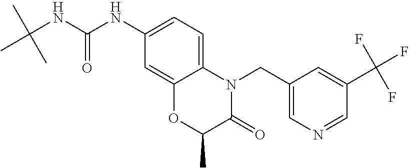 C₂₁H₂₃F₃N₄O₃ | (R)-1-(tert-butyl)-3-(2-methyl-3-oxo-4-((5- (trifluoromethyl)pyridin-3-yl)methyl)-3,4- dihydro-2H-benzo[b][1,4]oxazin-7-yl)urea |
| 172 | 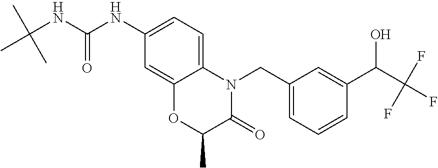 C₂₃H₂₆F₃N₃O₄ | 1-(tert-butyl)-3-((2R)-2-methyl-3-oxo-4-(3-(2,2,2- trifluoro-1-hydroxyethyl)benzyl)-3,4-dihydro-2H- benzo[b][1,4]oxazin-7-yl)urea |
| 173 | 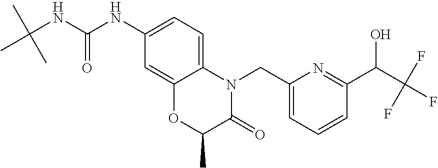 C₂₂H₂₅F₃N₄O₄ | 1-(tert-butyl)-3-((2R)-2-methyl-3-oxo-4-((6- (2,2,2-trifluoro-1-hydroxyethyl)pyridin-2- yl)methyl)-3,4-dihydro-2H-benzo[b][1,4]oxazin- 7-yl)urea |
| 174 | 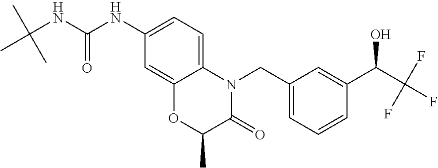 C₂₃H₂₆F₃N₃O₄ | 1-(tert-butyl)-3-((R)-2-methyl-3-oxo-4-(3-((R)- 2,2,2-trifluoro-1-hydroxyethyl)benzyl)-3,4- dihydro-2H-benzo[b][1,4]oxazin-7-yl)urea |
| 175 | 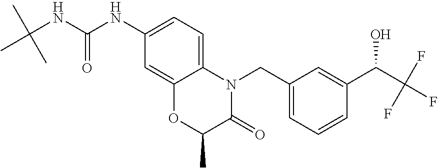 C₂₃H₂₆F₃N₃O₄ | 1-(tert-butyl)-3-((R)-2-methyl-3-oxo-4-(3-((S)- 2,2,2-trifluoro-1-hydroxyethyl)benzyl)-3,4- dihydro-2H-benzo[b][1,4]oxazin-7-yl)urea |
| 176 | 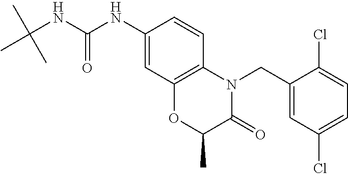 C₂₁H₂₃Cl₂N₃O₃ | (R)-1-(tert-butyl)-3-(4-(2,5-dichlorobenzyl)-2- methyl-3-oxo-3,4-dihydro-2H- benzo[b]\|1,4]oxazin-7-yl)urea |
| 177 | 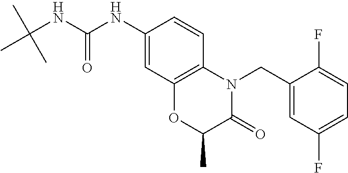 C₂₁H₂₃F₂N₃O₃ | (R)-1-(tert-butyl)-3-(4-(2,5-difluorobenzyl)-2- methyl-3-oxo-3,4-dihydro-2H- benzo[b][1,4]oxazin-7-yl)urea |
| 178 | 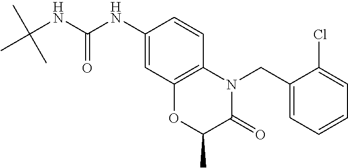 C₂₁H₂₄ClN₃O₃ | (R)-1-(tert-butyl)-3-(4-(2-chlorobenzyl)-2-methyl- 3-oxo-3,4-dihydro-2H-benzo[b][1,4]oxazin-7- yl)urea |
| 179 | 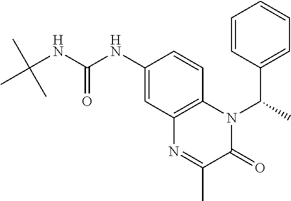 [SMILES: Cc1nc2cc(NC(=O)NC(C)(C)C)ccc2n([C@@H](C)c2ccccc2)c1=O] C₂₂H₂₆N₄O₂ | (S)-1-(tert-butyl)-3-(3-methyl-2-oxo-1-(1- phenylethyl)-1,2-dihydroquinoxalin-6-yl)urea |
| 180 | 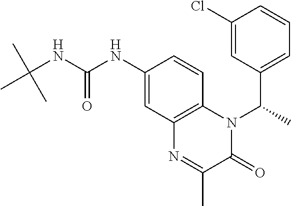 [SMILES: Cc1nc2cc(NC(=O)NC(C)(C)C)ccc2n([C@@H](C)c2cccc(Cl)c2)c1=O] C₂₂H₂₅ClN₄O₂ | (S)-1-(tert-butyl)-3-(1-(1-(3-chlorophenyl)ethyl)- 3-methyl-2-oxo-1,2-dihydroquinoxalin-6-yl)urea |
| 181 | 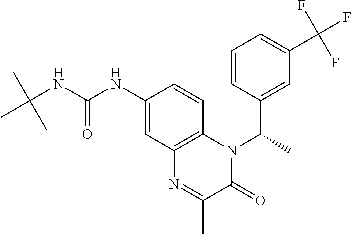 [SMILES: Cc1nc2cc(NC(=O)NC(C)(C)C)ccc2n([C@@H](C)c2cccc(C(F)(F)F)c2)c1=O] C₂₃H₂₅F₃N₄O₂ | (S)-1-(tert-butyl)-3-(3-methyl-2-oxo-1-(1-(3- (trifluoromethyl)phenyl)ethyl)-1,2- dihydroquinoxalin-6-yl)urea |
| 182 | 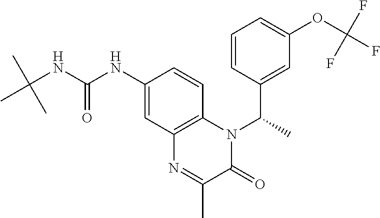 [SMILES: Cc1nc2cc(NC(=O)NC(C)(C)C)ccc2n([C@@H](C)c2cccc(OC(F)(F)F)c2)c1=O] C₂₃H₂₅F₃N₄O₃ | (S)-1-(tert-butyl)-3-(3-methyl-2-oxo-1-(1-(3- (trifluoromethoxy)phenyl)ethyl)-1,2- dihydroquinoxalin-6-yl)urea |
| 183 | 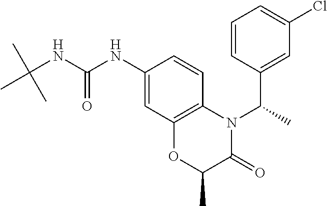 [SMILES: C[C@H]1Oc2cc(NC(=O)NC(C)(C)C)ccc2N([C@@H](C)c2cccc(Cl)c2)C1=O] C₂₂H₂₆ClN₃O₃ | 1-(tert-butyl)-3-((R)-4-((S)-1-(3- chlorophenyl)ethyl)-2-methyl-3-oxo-3,4-dihydro- 2H-benzo[b]\|1,4]oxazin-7-yl)urea |
| 184 | 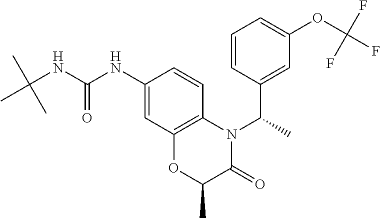 [SMILES: C[C@H]1Oc2cc(NC(=O)NC(C)(C)C)ccc2N([C@@H](C)c2cccc(OC(F)(F)F)c2)C1=O] C₂₃H₂₆F₃N₃O₄ | 1-(tert-butyl)-3-((R)-2-methyl-3-oxo-4-((S)-1-(3- (trifluoromethoxy)phenyl)ethyl)-3,4-dihydro-2H- benzo[b][1,4]oxazin-7-yl)urea |
| 185 | 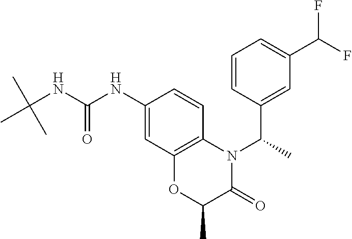 [SMILES: C[C@H]1Oc2cc(NC(=O)NC(C)(C)C)ccc2N([C@@H](C)c2cccc(C(F)F)c2)C1=O] C₂₃H₂₇F₂N₃O₃ | 1-(tert-butyl)-3-((R)-4-((S)-1-(3- (difluoromethyl)phenyl)ethyl)-2-methyl-3-oxo- 3,4-dihydro-2H-benzo[b][1,4]oxazin-7-yl)urea |
| 186 | 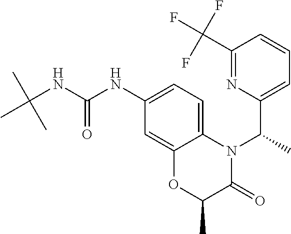 [SMILES: C[C@H]1Oc2cc(NC(=O)NC(C)(C)C)ccc2N([C@@H](C)c2cccc(C(F)(F)F)n2)C1=O] C₂₂H₂₅F₃N₄O₃ | 1-(tert-butyl)-3-((R)-2-methyl-3-oxo-4-((S)-1-(6- (trifluoromethyl)pyridin-2-yl)ethyl)-3,4-dihydro- 2H-benzo[b][1,4]oxazin-7-yl)urea |
| 187 | 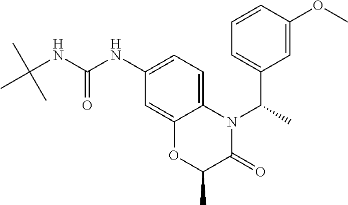 [SMILES: COc1cccc([C@H](C)N2C(=O)[C@@H](C)Oc3cc(NC(=O)NC(C)(C)C)ccc32)c1] C₂₃H₂₉N₃O₄ | 1-(tert-butyl)-3-((R)-4-((S)-1-(3- methoxyphenyl)ethyl)-2-methyl-3-oxo-3,4- dihydro-2H-benzo[b][1,4]oxazin-7-yl)urea |
| 188 | 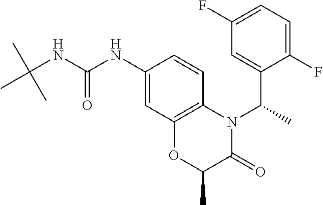 [SMILES: C[C@H]1Oc2cc(NC(=O)NC(C)(C)C)ccc2N([C@@H](C)c2cc(F)ccc2F)C1=O] C₂₂H₂₅F₂N₃O₃ | 1-(tert-butyl)-3-((R)-4-((S)-1-(2,5- difluorophenyl)ethyl)-2-methyl-3-oxo-3,4- dihydro-2H-benzo[b][1,4]oxazin-7-yl)urea |
| 189 | 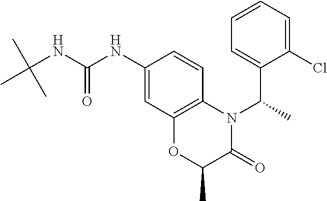 [SMILES: C[C@H]1Oc2cc(NC(=O)NC(C)(C)C)ccc2N([C@@H](C)c2ccccc2Cl)C1=O] C₂₂H₂₆ClN₃O₃ | 1-(tert-butyl)-3-((R)-4-((S)-1-(2- chlorophenyl)ethyl)-2-methyl-3-oxo-3,4-dihydro- 2H-benzo[b][1,4]oxazin-7-yl)urea |
| 190 | 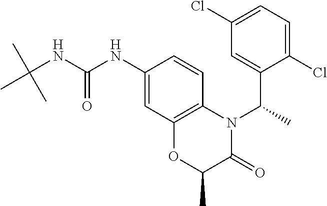 [SMILES: C[C@H]1Oc2cc(NC(=O)NC(C)(C)C)ccc2N([C@@H](C)c2cc(Cl)ccc2Cl)C1=O] C₂₂H₂₅Cl₂N₃O₃ | 1-(tert-butyl)-3-((R)-4-((S)-1-(2,5- dichlorophenyl)ethyl)-2-methyl-3-oxo-3,4- dihydro-2H-benzo[b][1,4]oxazin-7-yl)urea |
| 191 | 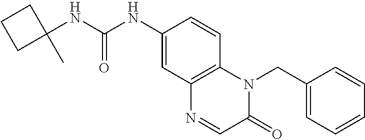 C₂₁H₂₂N₄O₂ | 1-(1-benzyl-2-oxo-1,2-dihydroquinoxalin-6-yl)-3- (1-methylcyclobutyl)urea |
| 192 | 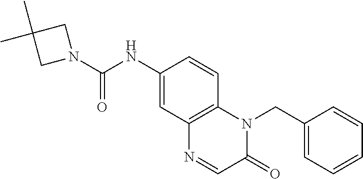 [SMILES: CC1(C)CN(C(=O)Nc2ccc3c(c2)ncc(=O)n3Cc2ccccc2)C1] C₂₁H₂₂N₄O₂ | N-(1-benzyl-2-oxo-1,2-dihydroquinoxalin-6-yl)- 3,3-dimethylazetidine-1-carboxamide |
| 193 | 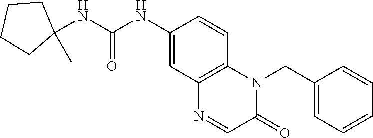 [SMILES: CC1(NC(=O)Nc2ccc3c(c2)ncc(=O)n3Cc2ccccc2)CCCC1] C₂₂H₂₄N₄O₂ | 1-(1-benzyl-2-oxo-1,2-dihydroquinoxalin-6-yl)-3- (1-methylcyclopentyl)urea |
| 194 | 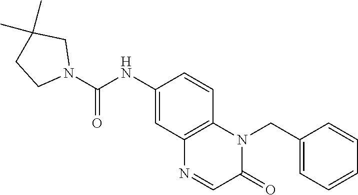 [SMILES: CC1(C)CCN(C(=O)Nc2ccc3c(c2)ncc(=O)n3Cc2ccccc2)C1] C₂₂H₂₄N₄O₂ | N-(1-benzyl-2-oxo-1,2-dihydroquinoxalin-6-yl)- 3,3-dimethylpyrrolidine-1-carboxamide |
| 195 | 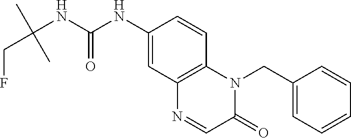 [SMILES: CC(C)(CF)NC(=O)Nc1ccc2c(c1)ncc(=O)n2Cc1ccccc1] C₂₀H₂₁FN₄O₂ | 1-(1-benzyl-2-oxo-1,2-dihydroquinoxalin-6-yl)-3- (1-fluoro-2-methylpropan-2-yl)urea |
| 196 | 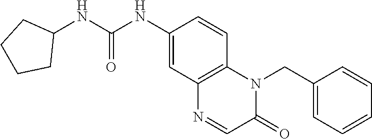 [SMILES: O=C(Nc1ccc2c(c1)ncc(=O)n2Cc1ccccc1)NC1CCCC1] C₂₁H₂₂N₄O₂ | 1-(1-benzyl-2-oxo-1,2-dihydroquinoxalin-6-yl)-3- cyclopentylurea |
| 197 | 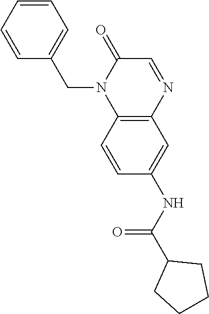 [SMILES: O=C(Nc1ccc2c(c1)ncc(=O)n2Cc1ccccc1)C1CCCC1] C₂₁H₂₁N₃O₂ | N-(1-benzyl-2-oxo-1,2-dihydroquinoxalin-6- yl)cyclopentanecarboxamide |
| 198 | 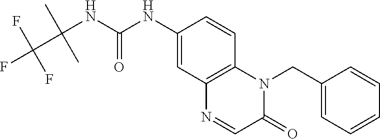 [SMILES: CC(C)(NC(=O)Nc1ccc2c(c1)ncc(=O)n2Cc1ccccc1)C(F)(F)F] C₂₀H₁₉F₃N₄O₂ | 1-(1-benzyl-2-oxo-1,2-dihydroquinoxalin-6-yl)-3- (1,1,1-trifluoro-2-methylpropan-2-yl)urea |
| 199 | 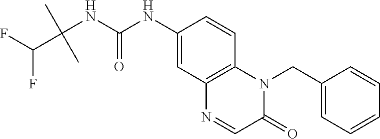 [SMILES: CC(C)(NC(=O)Nc1ccc2c(c1)ncc(=O)n2Cc1ccccc1)C(F)F] C₂₀H₂₀F₂N₄O₂ | 1-(1-benzyl-2-oxo-1,2-dihydroquinoxalin-6-yl)-3- (1,1-difluoro-2-methylpropan-2-yl)urea |
| 200 | 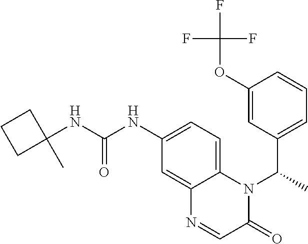 [SMILES: C[C@@H](c1cccc(OC(F)(F)F)c1)n1c(=O)cnc2cc(NC(=O)NC3(C)CCC3)ccc21] C₂₃H₂₃F₃N₄O₃ | (S)-1-(1-methylcyclobutyl)-3-(2-oxo-1-(1-(3- (trifluoromethoxy)phenyl)ethyl)-1,2- dihydroquinoxalin-6-yl)urea |
| 201 | 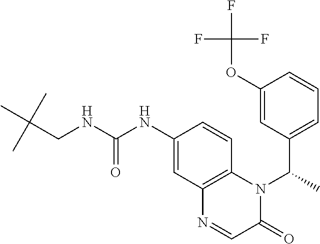 [SMILES: C[C@@H](c1cccc(OC(F)(F)F)c1)n1c(=O)cnc2cc(NC(=O)NCC(C)(C)C)ccc21] C₂₃H₂₅F₃N₄O₃ | (S)-1-neopentyl-3-(2-oxo-1-(1-(3- (trifluoromethoxy)phenyl)ethyl)-1,2- dihydroquinoxalin-6-yl)urea |
| 202 | 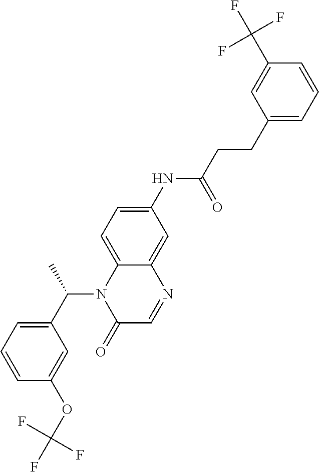 [SMILES: C[C@@H](c1cccc(OC(F)(F)F)c1)n1c(=O)cnc2cc(NC(=O)CCc3cccc(C(F)(F)F)c3)ccc21] C₂₇H₂₁F₆N₃O₃ | (S)-N-(2-oxo-1-(1-(3- (trifluoromethoxy)phenyl)ethyl)-1,2- dihydroquinoxalin-6-yl)-3-(3- (trifluoromethyl)phenyl)propanamide |
| 203 | 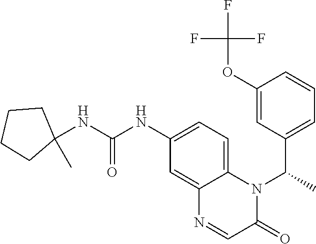 [SMILES: C[C@@H](c1cccc(OC(F)(F)F)c1)n1c(=O)cnc2cc(NC(=O)NC3(C)CCCC3)ccc21] C₂₄H₂₅F₃N₄O₃ | (S)-1-(1-methylcyclopentyl)-3-(2-oxo-1-(1-(3- (trifluoromethoxy)phenyl)ethyl)-1,2- dihydroquinoxalin-6-yl)urea |
| 204 | 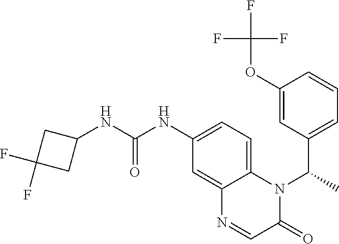 [SMILES: C[C@@H](c1cccc(OC(F)(F)F)c1)n1c(=O)cnc2cc(NC(=O)NC3CC(F)(F)C3)ccc21] C₂₂H₁₉F₅N₄O₃ | (S)-1-(3,3-difluorocyclobutyl)-3-(2-oxo-1-(1-(3- (trifluoromethoxy)phenyl)ethyl)-1,2- dihydroquinoxalin-6-yl)urea |
| 205 | 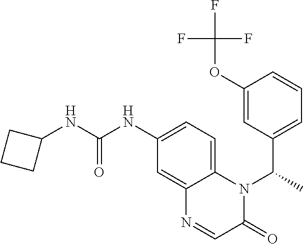 [SMILES: C[C@@H](c1cccc(OC(F)(F)F)c1)n1c(=O)cnc2cc(NC(=O)NC3CCC3)ccc21] C₂₂H₂₁F₃N₄O₃ | (S)-1-cyclobutyl-3-(2-oxo-1-(1-(3- (trifluoromethoxy)phenyl)ethyl)-1,2- dihydroquinoxalin-6-yl)urea |
| 206 | 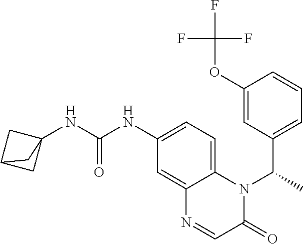 [SMILES: C[C@@H](c1cccc(OC(F)(F)F)c1)n1c(=O)cnc2cc(NC(=O)NC34CC(C3)C4)ccc21] C₂₃H₂₁F₃N₄O₃ | (S)-1-(bicyclo[1.1.1]pentan-1-yl)-3-(2-oxo-1-(1- (3-(trifluoromethoxy)phenyl)ethyl)-1,2- dihydroquinoxalin-6-yl)urea |
| 207 | 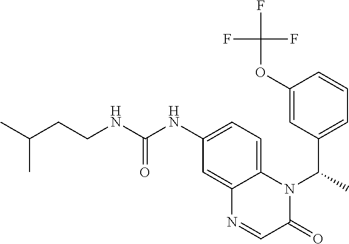 [SMILES: CC(C)CCNC(=O)Nc1ccc2c(c1)ncc(=O)n2[C@@H](C)c1cccc(OC(F)(F)F)c1] C₂₃H₂₅F₃N₄O₃ | (S)-1-isopentyl-3-(2-oxo-1-(1-(3- (trifluoromethoxy)phenyl)ethyl)-1,2- dihydroquinoxalin-6-yl)urea |
| 208 | 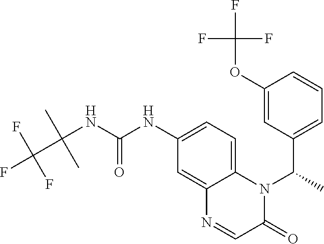 [SMILES: C[C@@H](c1cccc(OC(F)(F)F)c1)n1c(=O)cnc2cc(NC(=O)NC(C)(C)C(F)(F)F)ccc21] C₂₂H₂₀F₆N₄O₃ | (S)-1-(2-oxo-1-(1-(3- (trifluoromethoxy)phenyl)ethyl)-1,2- dihydroquinoxalin-6-yl)-3-(1,1,1-trifluoro-2- methylpropan-2-yl)urea |
| 209 | 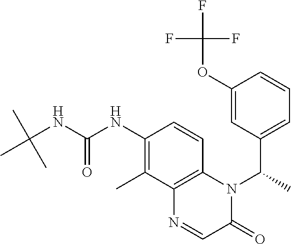 [SMILES: Cc1c(NC(=O)NC(C)(C)C)ccc2c1ncc(=O)n2[C@@H](C)c1cccc(OC(F)(F)F)c1] C₂₃H₂₅F₃N₄O₃ | (S)-1-(tert-butyl)-3-(5-methyl-2-oxo-1-(1-(3- (trifluoromethoxy)phenyl)ethyl)-1,2- dihydroquinoxalin-6-yl)urea |
| 210 | 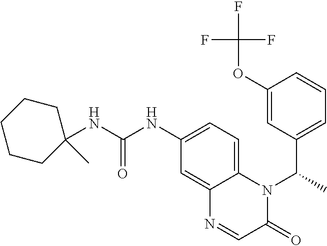 C₂₅H₂₇F₃N₄O₃ | (S)-1-(1-methylcyclohexyl)-3-(2-oxo-1-(1-(3- (trifluoromethoxy)phenyl)ethyl)-1,2- dihydroquinoxalin-6-yl)urea |
| 211 | 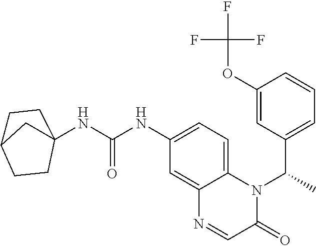 C₂₅H₂₅F₃N₄O₃ | (S)-1-(bicyclo[2.2.1]heptan-1-yl)-3-(2-oxo-1-(1- (3-(trifluoromethoxy)phenyl)ethyl)-1,2- dihydroquinoxalin-6-yl)urea |
| 212 | 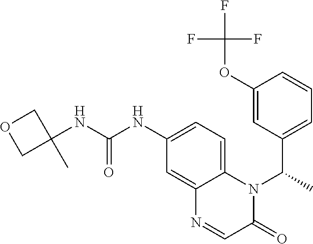 [SMILES: C[C@@H](c1cccc(OC(F)(F)F)c1)n1c(=O)cnc2cc(NC(=O)NC3(C)COC3)ccc21] C₂₂H₂₁F₃N₄O₄ | (S)-1-(3-methyloxetan-3-yl)-3-(2-oxo-1-(1-(3- (trifluoromethoxy)phenyl)ethyl)-1,2- dihydroquinoxalin-6-yl)urea |
| 213 | 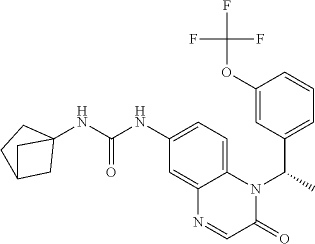 [SMILES: C[C@@H](c1cccc(OC(F)(F)F)c1)n1c(=O)cnc2cc(NC(=O)NC34CCC(C3)C4)ccc21] C₂₄H₂₃F₃N₄O₃ | (S)-1-(bicyclo[2.1.1]hexan-1-yl)-3-(2-oxo-1-(1-(3- (trifluoromethoxy)phenyl)ethyl)-1,2- dihydroquinoxalin-6-yl)urea |
| 214 | 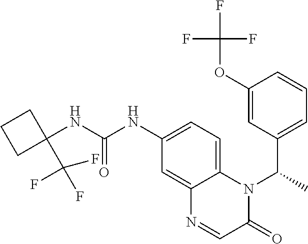 [SMILES: C[C@@H](c1cccc(OC(F)(F)F)c1)n1c(=O)cnc2cc(NC(=O)NC3(C(F)(F)F)CCC3)ccc21] C₂₃H₂₀F₆N₄O₃ | (S)-1-(2-oxo-1-(1-(3- (trifluoromethoxy)phenyl)ethyl)-1,2- dihydroquinoxalin-6-yl)-3-(1- (trifluoromethyl)cyclobutyl)urea |
| 215 | 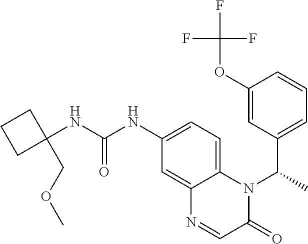 [SMILES: COCC1(NC(=O)Nc2ccc3c(c2)ncc(=O)n3[C@@H](C)c2cccc(OC(F)(F)F)c2)CCC1] C₂₄H₂₅F₃N₄O₄ | (S)-1-(1-(methoxymethyl)cyclobutyl)-3-(2-oxo-1- (1-(3-(trifluoromethoxy)phenyl)ethyl)-1,2- dihydroquinoxalin-6-yl)urea |
| 216 | 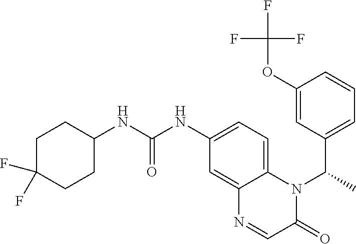 [SMILES: C[C@@H](c1cccc(OC(F)(F)F)c1)n1c(=O)cnc2cc(NC(=O)NC3CCC(F)(F)CC3)ccc21] C₂₄H₂₃F₅N₄O₃ | (S)-1-(4,4-difluorocyclohexyl)-3-(2-oxo-1-(1-(3- (trifluoromethoxy)phenyl)ethyl)-1,2- dihydroquinoxalin-6-yl)urea |
| 217 | 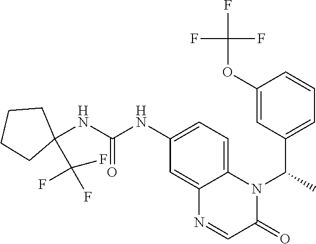 [SMILES: C[C@@H](c1cccc(OC(F)(F)F)c1)n1c(=O)cnc2cc(NC(=O)NC3(C(F)(F)F)CCCC3)ccc21] C₂₄H₂₂F₆N₄O₃ | (S)-1-(2-oxo-1-(1-(3- (trifluoromethoxy)phenyl)ethyl)-1,2- dihydroquinoxalin-6-yl)-3-(1- (trifluoromethyl)cyclopentyl)urea |
| 218 | 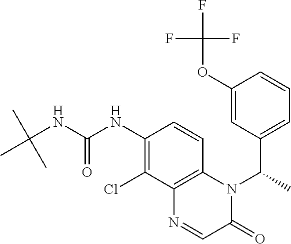 [SMILES: C[C@@H](c1cccc(OC(F)(F)F)c1)n1c(=O)cnc2c(Cl)c(NC(=O)NC(C)(C)C)ccc21] C₂₂H₂₂ClF₃N₄O₃ | (S)-1-(tert-butyl)-3-(5-chloro-2-oxo-1-(1-(3- (trifluoromethoxy)phenyl)ethyl)-1,2- dihydroquinoxalin-6-yl)urea |
| 219 | 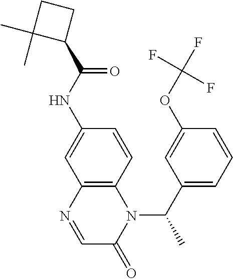 [SMILES: C[C@@H](c1cccc(OC(F)(F)F)c1)n1c(=O)cnc2cc(NC(=O)[C@@H]3CCC3(C)C)ccc21] C₂₄H₂₄F₃N₃O₃ | (R)-2,2-dimethyl-N-(2-oxo-1-((S)-1-(3- (trifluoromethoxy)phenyl)ethyl)-1,2- dihydroquinoxalin-6-yl)cyclobutane-1- carboxamide |
| 220 | 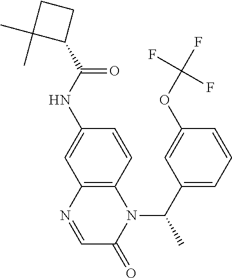 [SMILES: C[C@@H](c1cccc(OC(F)(F)F)c1)n1c(=O)cnc2cc(NC(=O)[C@H]3CCC3(C)C)ccc21] C₂₄H₂₄F₃N₃O₃ | (S)-2,2-dimethyl-N-(2-oxo-1-((S)-1-(3- (trifluoromethoxy)phenyl)ethyl)-1,2- dihydroquinoxalin-6-yl)cyclobutane-1- carboxamide |
| 221 | 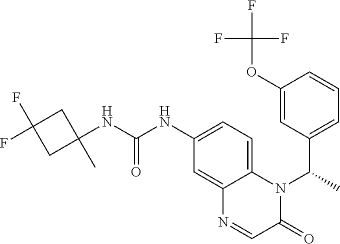 [SMILES: C[C@@H](c1cccc(OC(F)(F)F)c1)n1c(=O)cnc2cc(NC(=O)NC3(C)CC(F)(F)C3)ccc21] C₂₃H₂₁F₅N₄O₃ | (S)-1-(3,3-difluoro-1-methylcyclobutyl)-3-(2-oxo- 1-(1-(3-(trifluoromethoxy)phenyl)ethyl)-1,2- dihydroquinoxalin-6-yl)urea |
| 222 | 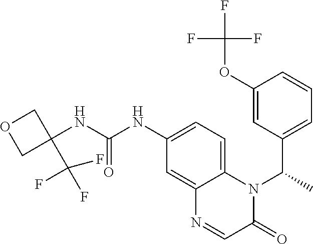 [SMILES: C[C@@H](c1cccc(OC(F)(F)F)c1)n1c(=O)cnc2cc(NC(=O)NC3(C(F)(F)F)COC3)ccc21] C₂₂H₁₈F₆N₄O₄ | (S)-1-(2-oxo-1-(1-(3- (trifluoromethoxy)phenyl)ethyl)-1,2- dihydroquinoxalin-6-yl)-3-(3- (trifluoromethyl)oxetan-3-yl)urea |
| 223 | 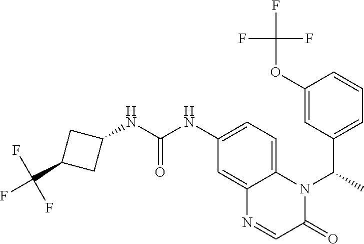 [SMILES: C[C@@H](c1cccc(OC(F)(F)F)c1)n1c(=O)cnc2cc(NC(=O)N[C@H]3C[C@H](C(F)(F)F)C3)ccc21] C₂₃H₂₀F₆N₄O₃ | 1-(2-oxo-1-((S)-1-(3- (trifluoromethoxy)phenyl)ethyl)-1,2- dihydroquinoxalin-6-yl)-3-((1r,3S)-3- (trifluoromethyl)cyclobutyl)urea |
| 224 | 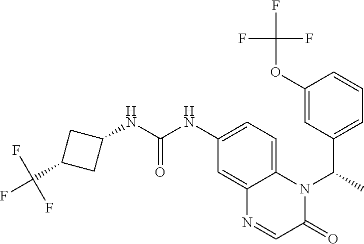 [SMILES: C[C@@H](c1cccc(OC(F)(F)F)c1)n1c(=O)cnc2cc(NC(=O)N[C@H]3C[C@@H](C(F)(F)F)C3)ccc21] C₂₃H₂₀F₆N₄O₃ | 1-(2-oxo-1-((S)-1-(3- (trifluoromethoxy)phenyl)ethyl)-1,2- dihydroquinoxalin-6-yl)-3-((1s,3R)-3- (trifluoromethyl)cyclobutyl)urea |
| 225 | 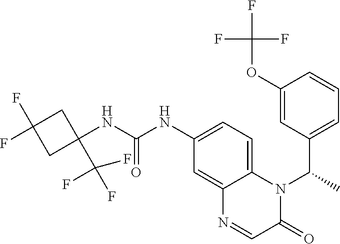 [SMILES: C[C@@H](c1cccc(OC(F)(F)F)c1)n1c(=O)cnc2cc(NC(=O)NC3(C(F)(F)F)CC(F)(F)C3)ccc21] C₂₃H₁₈F₈N₄O₃ | (S)-1-(3,3-difluoro-1- (trifluoromethyl)cyclobutyl)-3-(2-oxo-1-(1-(3- (trifluoromethoxy)phenyl)ethyl)-1,2- dihydroquinoxalin-6-yl)urea |
| 226 | 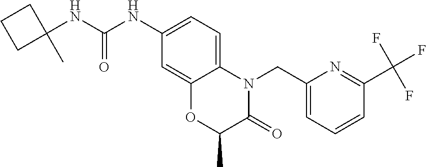 [SMILES: C[C@H]1Oc2cc(NC(=O)NC3(C)CCC3)ccc2N(Cc2cccc(C(F)(F)F)n2)C1=O] C₂₂H₂₃F₃N₄O₃ | (R)-1-(2-methyl-3-oxo-4-((6- (trifluoromethyl)pyridin-2-yl)methyl)-3,4- dihydro-2H-benzo[b][1,4]oxazin-7-yl)-3-(1- methylcyclobutyl)urea |
| 227 | 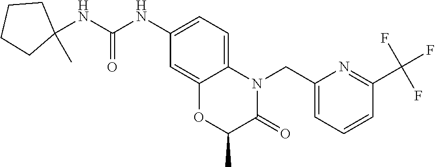 [SMILES: C[C@H]1Oc2cc(NC(=O)NC3(C)CCCC3)ccc2N(Cc2cccc(C(F)(F)F)n2)C1=O] C₂₃H₂₅F₃N₄O₃ | (R)-1-(2-methyl-3-oxo-4-((6- (trifluoromethyl)pyridin-2-yl)methyl)-3,4- dihydro-2H-benzo[b][1,4]oxazin-7-yl)-3-(1- methylcyclopentyl)urea |
| 228 | 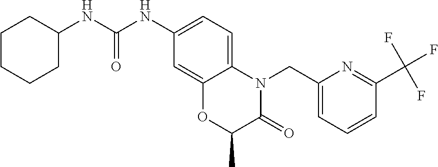 [SMILES: C[C@H]1Oc2cc(NC(=O)NC3CCCCC3)ccc2N(Cc2cccc(C(F)(F)F)n2)C1=O] C₂₃H₂₅F₃N₄O₃ | (R)-1-cyclohexyl-3-(2-methyl-3-oxo-4-((6- (trifluoromethyl)pyridin-2-yl)methyl)-3,4- dihydro-2H-benzo[b][1,4]oxazin-7-yl)urea |
| 229 | 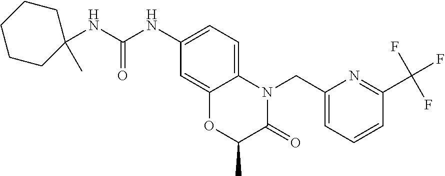 [SMILES: C[C@H]1Oc2cc(NC(=O)NC3(C)CCCCC3)ccc2N(Cc2cccc(C(F)(F)F)n2)C1=O] C₂₄H₂₇F₃N₄O₃ | (R)-1-(2-methyl-3-oxo-4-((6- (trifluoromethyl)pyridin-2-yl)methyl)-3,4- dihydro-2H-benzo[b][1,4]oxazin-7-yl)-3-(1- methylcyclohexyl)urea |
| 230 | 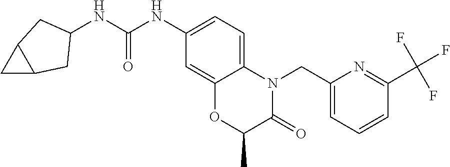 [SMILES: C[C@H]1Oc2cc(NC(=O)NC3CC4CC4C3)ccc2N(Cc2cccc(C(F)(F)F)n2)C1=O] C₂₃H₂₃F₃N₄O₃ | 1-(bicyclo[3.1.0]hexan-3-yl)-3-((R)-2-methyl-3- oxo-4-((6-(trifluoromethyl)pyridin-2-yl)methyl)- 3,4-dihydro-2H-benzo[b][1,4]oxazin-7-yl)urea |
| 231 | 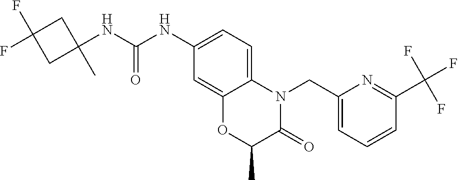 [SMILES: C[C@H]1Oc2cc(NC(=O)NC3(C)CC(F)(F)C3)ccc2N(Cc2cccc(C(F)(F)F)n2)C1=O] C₂₂H₂₁F₅N₄O₃ | (R)-1-(3,3-difluoro-1-methylcyclobutyl)-3-(2- methyl-3-oxo-4-((6-(trifluoromethyl)pyridin-2- yl)methyl)-3,4-dihydro-2H-benzo[b][1,4]oxazin- 7-yl)urea |
| 232 | 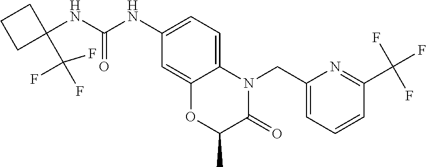 [SMILES: C[C@H]1Oc2cc(NC(=O)NC3(C(F)(F)F)CCC3)ccc2N(Cc2cccc(C(F)(F)F)n2)C1=O] C₂₂H₂₀F₆N₄O₃ | (R)-1-(2-methyl-3-oxo-4-((6- (trifluoromethyl)pyridin-2-yl)methyl)-3,4- dihydro-2H-benzo[b][1,4]oxazin-7-yl)-3-(1- (trifluoromethyl)cyclobutyl)urea |
| 233 | 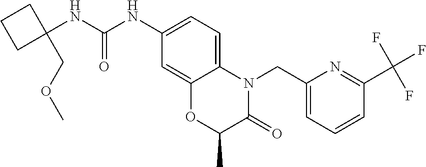 [SMILES: COCC1(NC(=O)Nc2ccc3c(c2)O[C@H](C)C(=O)N3Cc2cccc(C(F)(F)F)n2)CCC1] C₂₃H₂₅F₃N₄O₄ | (R)-1-(1-(methoxymethyl)cyclobutyl)-3-(2- methyl-3-oxo-4-((6-(trifluoromethyl)pyridin-2- yl)methyl)-3,4-dihydro-2H-benzo[b][1,4]oxazin- 7-yl)urea |
| 234 | 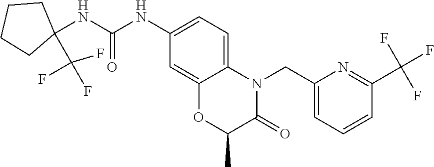 [SMILES: C[C@H]1Oc2cc(NC(=O)NC3(C(F)(F)F)CCCC3)ccc2N(Cc2cccc(C(F)(F)F)n2)C1=O] C₂₃H₂₂F₆N₄O₃ | (R)-1-(2-methyl-3-oxo-4-((6- (trifluoromethyl)pyridin-2-yl)methyl)-3,4- dihydro-2H-benzo[b][1,4]oxazin-7-yl)-3-(1- (trifluoromethyl)cyclopentyl)urea |
| 235 | 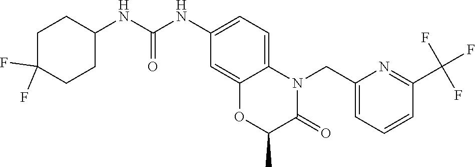 [SMILES: C[C@H]1Oc2cc(NC(=O)NC3CCC(F)(F)CC3)ccc2N(Cc2cccc(C(F)(F)F)n2)C1=O] C₂₃H₂₃F₅N₄O₃ | (R)-1-(4,4-difluorocyclohexyl)-3-(2-methyl-3- oxo-4-((6-(trifluoromethyl)pyridin-2-yl)methyl)- 3,4-dihydro-2H-benzo[b][1,4]oxazin-7-yl)urea |
| 236 | 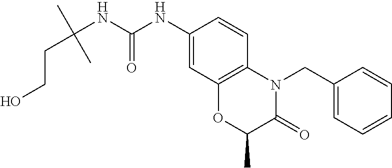 C₂₂H₂₇N₃O₄ | (R)-1-(4-benzyl-2-methyl-3-oxo-3,4-dihydro-2H- benzo[b][1,4]oxazin-7-yl)-3-(4-hydroxy-2- methylbutan-2-yl)urea |
| 237 | 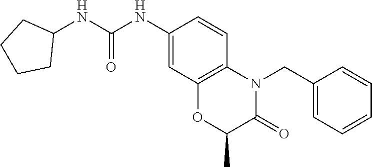 [SMILES: C[C@H]1Oc2cc(NC(=O)NC3CCCC3)ccc2N(Cc2ccccc2)C1=O] C₂₂H₂₅N₃O₃ | (R)-1-(4-benzyl-2-methyl-3-oxo-3,4-dihydro-2H- benzo[b][1,4]oxazin-7-yl)-3-cyclopentylurea |
| 238 | 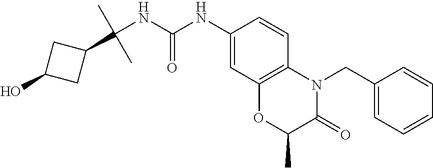 C₂₄H₂₉N₃O₄ | 1-((R)-4-benzyl-2-methyl-3-oxo-3,4-dihydro-2H- benzo[b][1,4]oxazin-7-yl)-3-(2-((1s,3S)-3- hydroxycyclobutyl)propan-2-yl)urea |
| 239 | 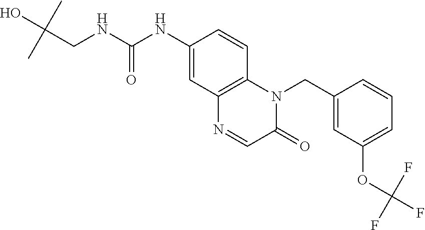 [SMILES: CC(C)(O)CNC(=O)Nc1ccc2c(c1)ncc(=O)n2Cc1cccc(OC(F)(F)F)c1] C₂₁H₂₁F₃N₄O₄ | 1-(2-hydroxy-2-methylpropyl)-3-(2-oxo-1-(3- (trifluoromethoxy)benzyl)-1,2-dihydroquinoxalin- 6-yl)urea |
| 240 | 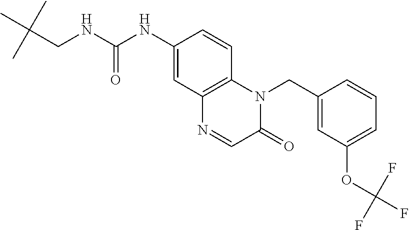 [SMILES: CC(C)(C)CNC(=O)Nc1ccc2c(c1)ncc(=O)n2Cc1cccc(OC(F)(F)F)c1] C₂₂H₂₃F₃N₄O₃ | 1-neopentyl-3-(2-oxo-1-(3- (trifluoromethoxy)benzyl)-1,2-dihydroquinoxalin- 6-yl)urea |
| 241 | 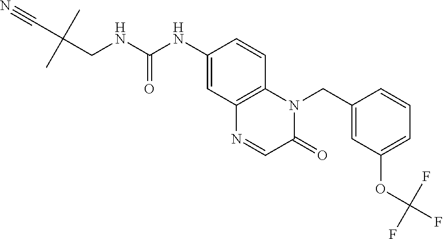 [SMILES: CC(C)(C#N)CNC(=O)Nc1ccc2c(c1)ncc(=O)n2Cc1cccc(OC(F)(F)F)c1] C₂₂H₂₀F₃N₅O₃ | 1-(2-cyano-2-methylpropyl)-3-(2-oxo-1-(3- (trifluoromethoxy)benzyl)-1,2-dihydroquinoxalin- 6-yl)urea |
| 242 | 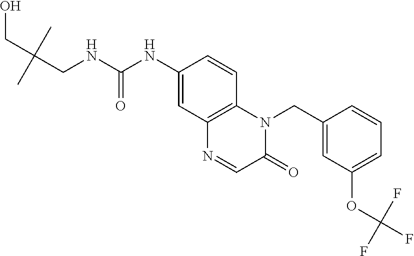 [SMILES: CC(C)(CO)CNC(=O)Nc1ccc2c(c1)ncc(=O)n2Cc1cccc(OC(F)(F)F)c1] C₂₂H₂₃F₃N₄O₄ | 1-(3-hydroxy-2,2-dimethylpropyl)-3-(2-oxo-1-(3- (trifluoromethoxy)benzyl)-1,2-dihydroquinoxalin- 6-yl)urea |
| 243 | 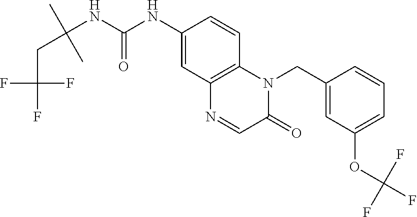 [SMILES: CC(C)(CC(F)(F)F)NC(=O)Nc1ccc2c(c1)ncc(=O)n2Cc1cccc(OC(F)(F)F)c1] C₂₂H₂₀F₆N₄O₃ | 1-(2-oxo-1-(3-(trifluoromethoxy)benzyl)-1,2- dihydroquinoxalin-6-yl)-3-(4,4,4-trifluoro-2- methylbutan-2-yl)urea |
| 244 | 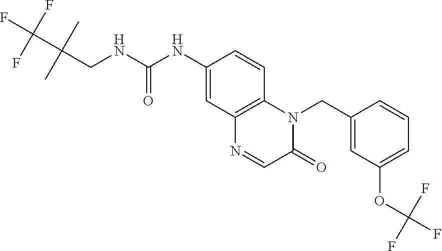 [SMILES: CC(C)(CNC(=O)Nc1ccc2c(c1)ncc(=O)n2Cc1cccc(OC(F)(F)F)c1)C(F)(F)F] C₂₂H₂₀F₆N₄O₃ | 1-(2-oxo-1-(3-(trifluoromethoxy)benzyl)-1,2- dihydroquinoxalin-6-yl)-3-(3,3,3-trifluoro-2,2- dimethylpropyl)urea |
| 245 | 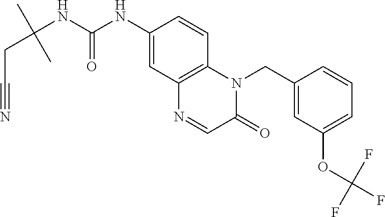 [SMILES: CC(C)(CC#N)NC(=O)Nc1ccc2c(c1)ncc(=O)n2Cc1cccc(OC(F)(F)F)c1] C₂₂H₂₀F₃N₅O₃ | 1-(1-cyano-2-methylpropan-2-yl)-3-(2-oxo-1-(3- (trifluoromethoxy)benzyl)-1,2-dihydroquinoxalin- 6-yl)urea |
| 246 | 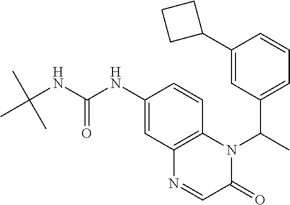 [SMILES: CC(c1cccc(C2CCC2)c1)n1c(=O)cnc2cc(NC(=O)NC(C)(C)C)ccc21] C₂₅H₃₀N₄O₂ | 1-(tert-butyl)-3-(1-(1-(3-cyclobutylphenyl)ethyl)- 2-oxo-1,2-dihydroquinoxalin-6-yl)urea |
| 247 | 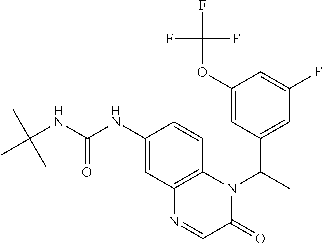 [SMILES: CC(c1cc(F)cc(OC(F)(F)F)c1)n1c(=O)cnc2cc(NC(=O)NC(C)(C)C)ccc21] C₂₂H₂₂F₄N₄O₃ | 1-(tert-butyl)-3-(1-(1-(3-fluoro-5- (trifluoromethoxy)phenyl)ethyl)-2-oxo-1,2- dihydroquinoxalin-6-yl)urea |
| 248 | 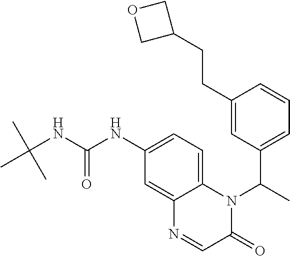 [SMILES: CC(c1cccc(CCC2COC2)c1)n1c(=O)cnc2cc(NC(=O)NC(C)(C)C)ccc21] C₂₆H₃₂N₄O₃ | 1-(tert-butyl)-3-(1-(1-(3-(2-(oxetan-3- yl)ethyl)phenyl)ethyl)-2-oxo-1,2- dihydroquinoxalin-6-yl)urea |
| 249 | 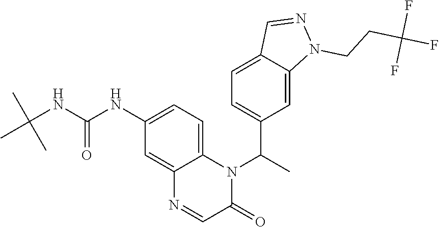 [SMILES: CC(c1ccc2cnn(CCC(F)(F)F)c2c1)n1c(=O)cnc2cc(NC(=O)NC(C)(C)C)ccc21] C₂₅H₂₇F₃N₆O₂ | 1-(tert-butyl)-3-(2-oxo-1-(1-(1-(3,3,3- trifluoropropyl)-1H-indazol-6-yl)ethyl)-1,2- dihydroquinoxalin-6-yl)urea |
| 250 | 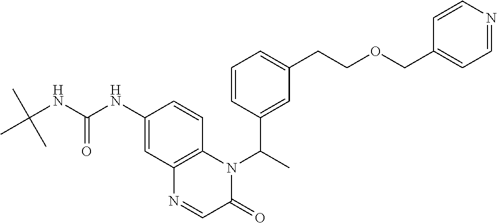 [SMILES: CC(c1cccc(CCOCc2ccncc2)c1)n1c(=O)cnc2cc(NC(=O)NC(C)(C)C)ccc21] C₂₉H₃₃N₅O₃ | 1-(tert-butyl)-3-(2-oxo-1-(1-(3-(2-(pyridin-4- ylmethoxy)ethyl)phenyl)ethyl)-1,2- dihydroquinoxalin-6-yl)urea |
| 251 | 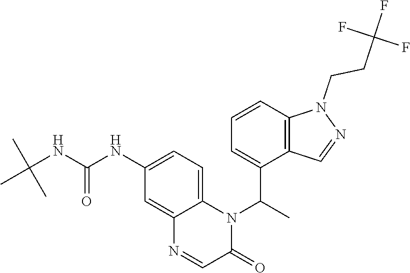 [SMILES: CC(c1cccc2c1cnn2CCC(F)(F)F)n1c(=O)cnc2cc(NC(=O)NC(C)(C)C)ccc21] C₂₅H₂₇F₃N₆O₂ | 1-(tert-butyl)-3-(2-oxo-1-(1-(1-(3,3,3- trifluoropropyl)-1H-indazol-4-yl)ethyl)-1,2- dihydroquinoxalin-6-yl)urea |
| 252 | 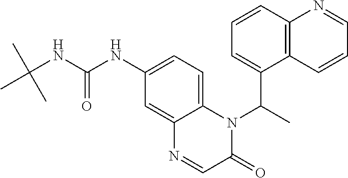 [SMILES: CC(c1cccc2ncccc12)n1c(=O)cnc2cc(NC(=O)NC(C)(C)C)ccc21] C₂₄H₂₅N₅O₂ | 1-(tert-butyl)-3-(2-oxo-1-(1-(quinolin-5-yl)ethyl)- 1,2-dihydroquinoxalin-6-yl)urea |
| 253 | 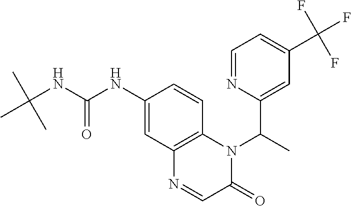 [SMILES: CC(c1cc(C(F)(F)F)ccn1)n1c(=O)cnc2cc(NC(=O)NC(C)(C)C)ccc21] C₂₁H₂₂F₃N₅O₂ | 1-(tert-butyl)-3-(2-oxo-1-(1-(4- (trifluoromethyl)pyridin-2-yl)ethyl)-1,2- dihydroquinoxalin-6-yl)urea |
| 254 | 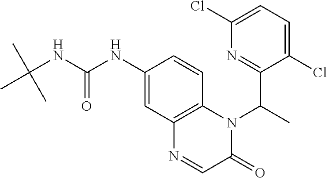 [SMILES: CC(c1nc(Cl)ccc1Cl)n1c(=O)cnc2cc(NC(=O)NC(C)(C)C)ccc21] C₂₀H₂₁Cl₂N₅O₂ | 1-(tert-butyl)-3-(1-(1-(3,6-dichloropyridin-2- yl)ethyl)-2-oxo-1,2-dihydroquinoxalin-6-yl)urea |
| 255 | 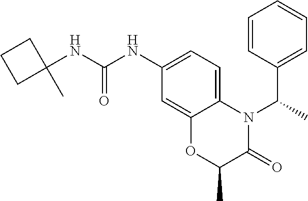 [SMILES: C[C@H]1Oc2cc(NC(=O)NC3(C)CCC3)ccc2N([C@@H](C)c2ccccc2)C1=O] C₂₃H₂₇N₃O₃ | 1-((R)-2-methyl-3-oxo-4-((S)-1-phenylethyl)-3,4- dihydro-2H-benzo[b][1,4]oxazin-7-yl)-3-(1- methylcyclobutyl)urea |
| 256 | 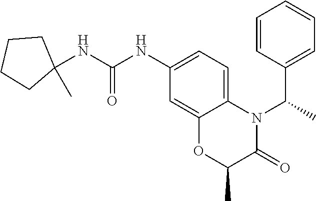 [SMILES: C[C@H]1Oc2cc(NC(=O)NC3(C)CCCC3)ccc2N([C@@H](C)c2ccccc2)C1=O] C₂₄H₂₉N₃O₃ | 1-((R)-2-methyl-3-oxo-4-((S)-1-phenylethyl)-3,4- dihydro-2H-benzo[b][1,4]oxazin-7-yl)-3-(1- methylcyclopentyl)urea |
| 257 | 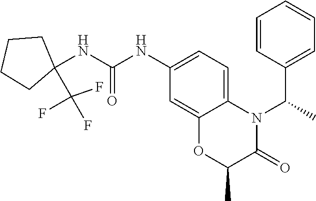 [SMILES: C[C@H]1Oc2cc(NC(=O)NC3(C(F)(F)F)CCCC3)ccc2N([C@@H](C)c2ccccc2)C1=O] C₂₄H₂₆F₃N₃O₃ | 1-((R)-2-methyl-3-oxo-4-((S)-1-phenylethyl)-3,4- dihydro-2H-benzo[b][1,4]oxazin-7-yl)-3-(1- (trifluoromethyl)cyclopentyl)urea |
| 258 | 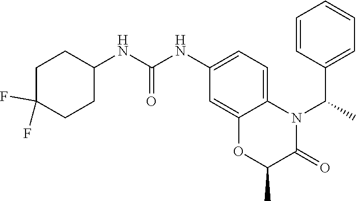 [SMILES: C[C@H]1Oc2cc(NC(=O)NC3CCC(F)(F)CC3)ccc2N([C@@H](C)c2ccccc2)C1=O] C₂₄H₂₇F₂N₃O₃ | 1-(4,4-difluorocyclohexyl)-3-((R)-2-methyl-3- oxo-4-((S)-1-phenylethyl)-3,4-dihydro-2H- benzo[b][1,4]oxazin-7-yl)urea |
| 259 | 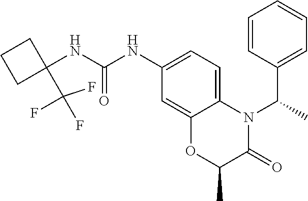 [SMILES: C[C@H]1Oc2cc(NC(=O)NC3(C(F)(F)F)CCC3)ccc2N([C@@H](C)c2ccccc2)C1=O] C₂₃H₂₄F₃N₃O₃ | 1-((R)-2-methyl-3-oxo-4-((S)-1-phenylethyl)-3,4- dihydro-2H-benzo[b][1,4]oxazin-7-yl)-3-(1- (trifluoromethyl)cyclobutyl)urea |
| 260 | 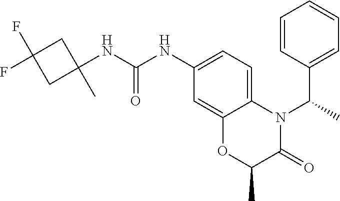 [SMILES: C[C@H]1Oc2cc(NC(=O)NC3(C)CC(F)(F)C3)ccc2N([C@@H](C)c2ccccc2)C1=O] C₂₃H₂₅F₂N₃O₃ | 1-(3,3-difluoro-1-methylcyclobutyl)-3-((R)-2- methyl-3-oxo-4-((S)-1-phenylethyl)-3,4-dihydro- 2H-benzo[b][1,4]oxazin-7-yl)urea |
| 261 | 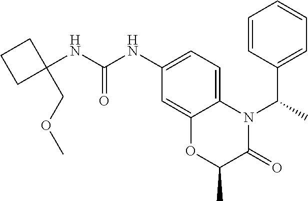 [SMILES: COCC1(NC(=O)Nc2ccc3c(c2)O[C@H](C)C(=O)N3[C@@H](C)c2ccccc2)CCC1] C₂₄H₂₉N₃O₄ | 1-(1-(methoxymethyl)cyclobutyl)-3-((R)-2- methyl-3-oxo-4-((S)-1-phenylethyl)-3,4-dihydro- 2H-benzo[b][1,4]oxazin-7-yl)urea |
| 262 | 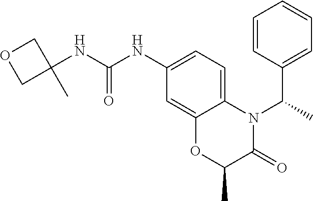 [SMILES: C[C@H]1Oc2cc(NC(=O)NC3(C)COC3)ccc2N([C@@H](C)c2ccccc2)C1=O] C₂₂H₂₅N₃O₄ | 1-((R)-2-methyl-3-oxo-4-((S)-1-phenylethyl)-3,4- dihydro-2H-benzo[b][1,4]oxazin-7-yl)-3-(3- methyloxetan-3-yl)urea |
| 263 | 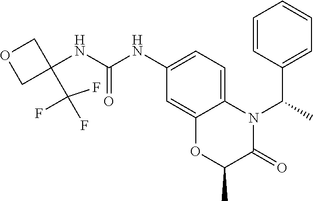 [SMILES: C[C@H]1Oc2cc(NC(=O)NC3(C(F)(F)F)COC3)ccc2N([C@@H](C)c2ccccc2)C1=O] C₂₂H₂₂F₃N₃O₄ | 1-((R)-2-methyl-3-oxo-4-((S)-1-phenylethyl)-3,4- dihydro-2H-benzo[b][1,4]oxazin-7-yl)-3-(3- (trifluoromethyl)oxetan-3-yl)urea |
| 264 | 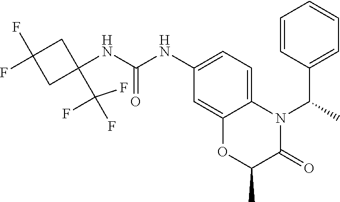 [SMILES: C[C@H]1Oc2cc(NC(=O)NC3(C(F)(F)F)CC(F)(F)C3)ccc2N([C@@H](C)c2ccccc2)C1=O] C₂₃H₂₂F₅N₃O₃ | 1-(3,3-difluoro-1-(trifluoromethyl)cyclobutyl)-3- ((R)-2-methyl-3-oxo-4-((S)-1-phenylethyl)-3,4- dihydro-2H-benzo[b][1,4]oxazin-7-yl)urea |
| 265 | 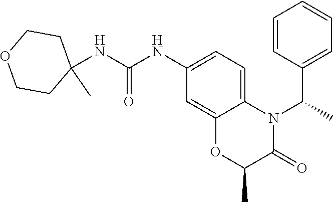 [SMILES: C[C@H]1Oc2cc(NC(=O)NC3(C)CCOCC3)ccc2N([C@@H](C)c2ccccc2)C1=O] C₂₄H₂₉N₃O₄ | 1-((R)-2-methyl-3-oxo-4-((S)-1-phenylethyl)-3,4- dihydro-2H-benzo[b][1,4]oxazin-7-yl)-3-(4- methyltetrahydro-2H-pyran-4-yl)urea |
| 266 | 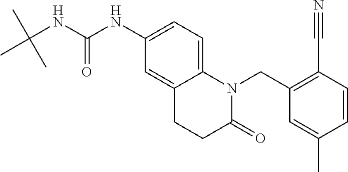 [SMILES: Cc1ccc(C#N)c(CN2C(=O)CCc3cc(NC(=O)NC(C)(C)C)ccc32)c1] C₂₃H₂₆N₄O₂ | 1-(tert-butyl)-3-(1-(2-cyano-5-methylbenzyl)-2- oxo-1,2,3,4-tetrahydroquinolin-6-yl)urea |
| 267 | 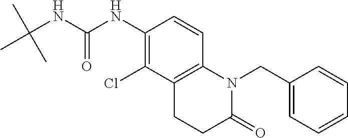 [SMILES: CC(C)(C)NC(=O)Nc1ccc2c(c1Cl)CCC(=O)N2Cc1ccccc1] C₂₁H₂₄ClN₃O₂ | 1-(1-benzyl-5-chloro-2-oxo-1,2,3,4- tetrahydroquinolin-6-yl)-3-(tert-butyl)urea |
| 268 | 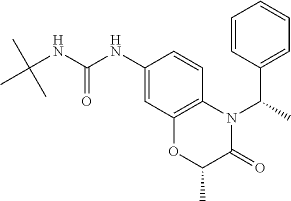 [SMILES: C[C@@H]1Oc2cc(NC(=O)NC(C)(C)C)ccc2N([C@@H](C)c2ccccc2)C1=O] C₂₂H₂₇N₃O₃ | 1-(tert-butyl)-3-((S)-2-methyl-3-oxo-4-((S)-1- phenylethyl)-3,4-dihydro-2H- benzo[b][1,4]oxazin-7-yl)urea |
| 269 | 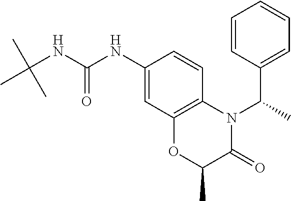 [SMILES: C[C@H]1Oc2cc(NC(=O)NC(C)(C)C)ccc2N([C@@H](C)c2ccccc2)C1=O] C₂₂H₂₇N₃O₃ | 1-(tert-butyl)-3-((R)-2-methyl-3-oxo-4-((S)-1- phenylethyl)-3,4-dihydro-2H- benzo[b][1,4]oxazin-7-yl)urea |
| 270 | 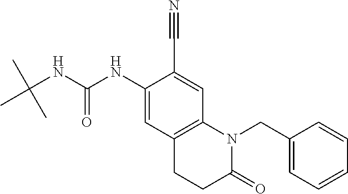 [SMILES: CC(C)(C)NC(=O)Nc1cc2c(cc1C#N)N(Cc1ccccc1)C(=O)CC2] C₂₂H₂₄N₄O₂ | 1-(1-benzyl-7-cyano-2-oxo-1,2,3,4- tetrahydroquinolin-6-yl)-3-(tert-butyl)urea |
| 271 | 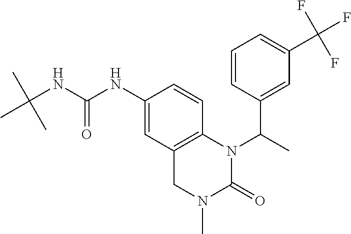 [SMILES: CC(c1cccc(C(F)(F)F)c1)N1C(=O)N(C)Cc2cc(NC(=O)NC(C)(C)C)ccc21] C₂₃H₂₇F₃N₄O₂ | 1-(tert-butyl)-3-(3-methyl-2-oxo-1-(1-(3- (trifluoromethyl)phenyl)ethyl)-1,2,3,4- tetrahydroquinazolin-6-yl)urea |
| 272 | 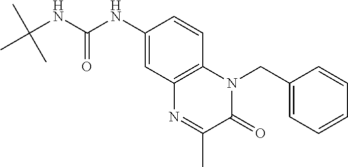 [SMILES: Cc1nc2cc(NC(=O)NC(C)(C)C)ccc2n(Cc2ccccc2)c1=O] C₂₁H₂₄N₄O₂ | 1-(1-benzyl-3-methyl-2-oxo-1,2- dihydroquinoxalin-6-yl)-3-(tert-butyl)urea |
| 273 | 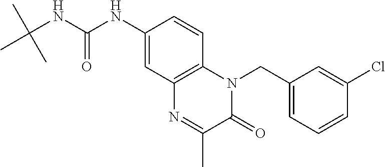 [SMILES: Cc1nc2cc(NC(=O)NC(C)(C)C)ccc2n(Cc2cccc(Cl)c2)c1=O] C₂₁H₂₃ClN₄O₂ | 1-(tert-butyl)-3-(1-(3-chlorobenzyl)-3-methyl-2- oxo-1,2-dihydroquinoxalin-6-yl)urea |
| 274 | 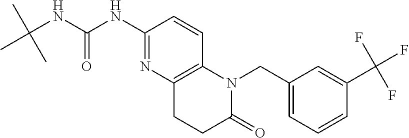 [SMILES: CC(C)(C)NC(=O)Nc1ccc2c(n1)CCC(=O)N2Cc1cccc(C(F)(F)F)c1] C₂₁H₂₃F₃N₄O₂ | 1-(tert-butyl)-3-(6-oxo-5-(3- (trifluoromethyl)benzyl)-5,6,7,8-tetrahydro-1,5- naphthyridin-2-yl)urea |
| 275 | 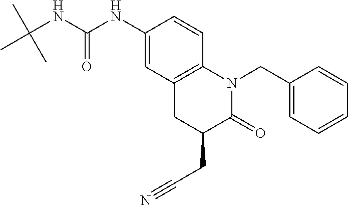 [SMILES: CC(C)(C)NC(=O)Nc1ccc2c(c1)C[C@H](CC#N)C(=O)N2Cc1ccccc1] C₂₃H₂₆N₄O₂ | (R)-1-(1-benzyl-3-(cyanomethyl)-2-oxo-1,2,3,4- tetrahydroquinolin-6-yl)-3-(tert-butyl)urea |
| 276 | 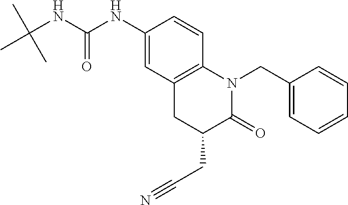 [SMILES: CC(C)(C)NC(=O)Nc1ccc2c(c1)C[C@@H](CC#N)C(=O)N2Cc1ccccc1] C₂₃H₂₆N₄O₂ | (S)-1-(1-benzyl-3-(cyanomethyl)-2-oxo-1,2,3,4- tetrahydroquinolin-6-yl)-3-(tert-butyl)urea |
| 277 | 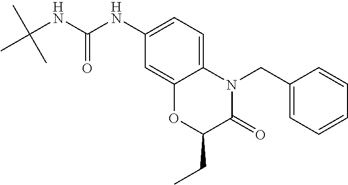 C₂₂H₂₇N₃O₃ | (R)-1-(4-benzyl-2-ethyl-3-oxo-3,4-dihydro-2H- benzo[b][1,4]oxazin-7-yl)-3-(tert-butyl)urea |
| 278 | 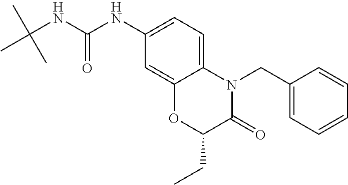 C₂₂H₂₇N₃O₃ | (S)-1-(4-benzyl-2-ethyl-3-oxo-3,4-dihydro-2H- benzo[b][1,4]oxazin-7-yl)-3-(tert-butyl)urea |
| 279 | 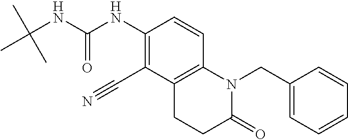 C₂₂H₂₄N₄O₂ | 1-(1-benzyl-5-cyano-2-oxo-1,2,3,4- tetrahydroquinolin-6-yl)-3-(tert-butyl)urea |
| 280 | 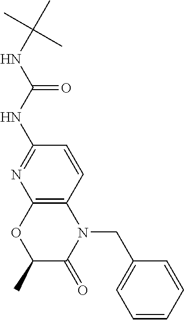 [SMILES: C[C@H]1Oc2nc(NC(=O)NC(C)(C)C)ccc2N(Cc2ccccc2)C1=O] C₂₀H₂₄N₄O₃ | (R)-1-(1-benzyl-3-methyl-2-oxo-2,3-dihydro-1H- pyrido[2,3-b][1,4]oxazin-6-yl)-3-(tert-butyl)urea |
| 281 | 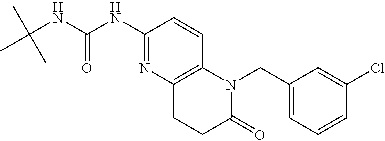 C₂₀H₂₃ClN₄O₂ | 1-(tert-butyl)-3-(5-(3-chlorobenzyl)-6-oxo- 5,6,7,8-tetrahydro-1,5-naphthyridin-2-yl)urea |
| 282 | 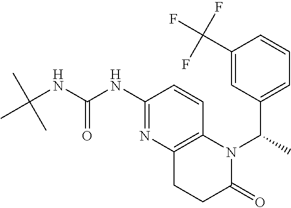 C₂₂H₂₅F₃N₄O₂ | (S)-1-(tert-butyl)-3-(6-oxo-5-(1-(3- (trifluoromethyl)phenyl)ethyl)-5,6,7,8-tetrahydro- 1,5-naphthyridin-2-yl)urea |
| 283 | 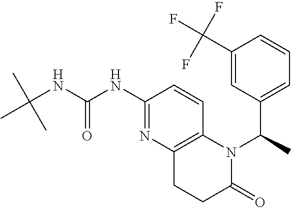 C₂₂H₂₅F₃N₄O₂ | (R)-1-(tert-butyl)-3-(6-oxo-5-(1-(3- (trifluoromethyl)phenyl)ethyl)-5,6,7,8-tetrahydro- 1,5-naphthyridin-2-yl)urea |
| 284 | 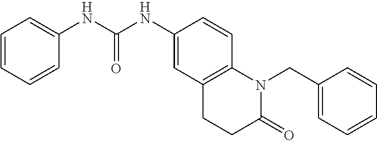 C₂₃H₂₁N₃O₂ | 1-(1-benzyl-2-oxo-1,2,3,4-tetrahydroquinolin-6- yl)-3-phenylurea |
| 285 | 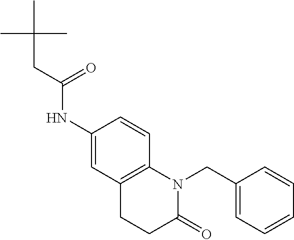 [SMILES: CC(C)(C)CC(=O)Nc1ccc2c(c1)CCC(=O)N2Cc1ccccc1] C₂₂H₂₆N₂O₂ | N-(1-benzyl-2-oxo-1,2,3,4-tetrahydroquinolin-6- yl)-3,3-dimethylbutanamide |
| 286 | 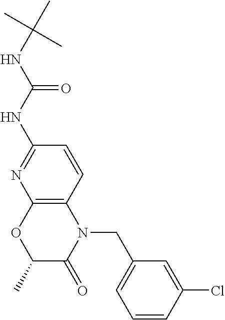 [SMILES: C[C@@H]1Oc2nc(NC(=O)NC(C)(C)C)ccc2N(Cc2cccc(Cl)c2)C1=O] C₂₀H₂₃ClN₄O₃ | (S)-1-(tert-butyl)-3-(1-(3-chlorobenzyl)-3-methyl- 2-oxo-2,3-dihydro-1H-pyrido[2,3-b][1,4]oxazin- 6-yl)urea |
| 287 | 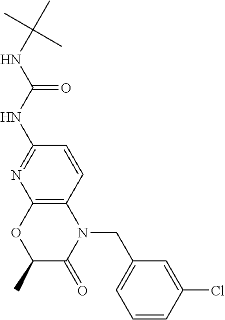 [SMILES: C[C@H]1Oc2nc(NC(=O)NC(C)(C)C)ccc2N(Cc2cccc(Cl)c2)C1=O] C₂₀H₂₃ClN₄O₃ | (R)-1-(tert-butyl)-3-(1-(3-chlorobenzyl)-3-methyl- 2-oxo-2,3-dihydro-1H-pyrido[2,3-b][1,4]oxazin- 6-yl)urea |
| 288 | 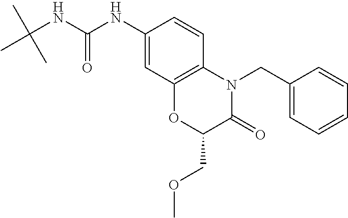 C₂₂H₂₇N₃O₄ | (S)-1-(4-benzyl-2-(methoxymethyl)-3-oxo-3,4- dihydro-2H-benzo[b][1,4]oxazin-7-yl)-3-(tert- butyl)urea |
| 289 | 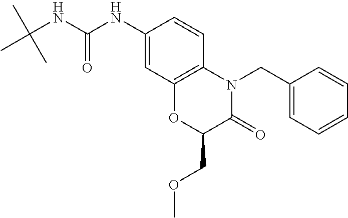 C₂₂H₂₇N₃O₄ | (R)-1-(4-benzyl-2-(methoxymethyl)-3-oxo-3,4- dihydro-2H-benzo[b][1,4]oxazin-7-yl)-3-(tert- butyl)urea |
| 290 | 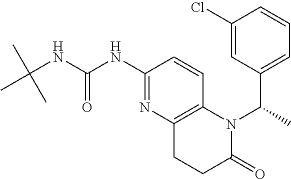 C₂₁H₂₅ClN₄O₂ | (S)-1-(tert-butyl)-3-(5-(1-(3-chlorophenyl)ethyl)- 6-oxo-5,6,7,8-tetrahydro-1,5-naphthyridin-2- yl)urea |
| 291 | 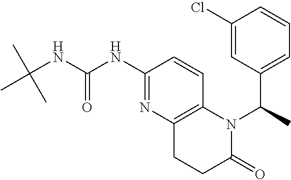 C₂₁H₂₅ClN₄O₂ | (R)-1-(tert-butyl)-3-(5-(1-(3-chlorophenyl)ethyl)- 6-oxo-5,6,7,8-tetrahydro-1,5-naphthyridin-2- yl)urea |
| 292 | 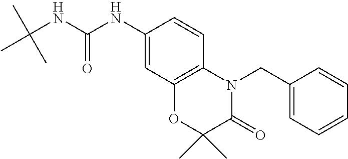 C₂₂H₂₇N₃O₃ | 1-(4-benzyl-2,2-dimethyl-3-oxo-3,4-dihydro-2H- benzo[b]\|1,4]oxazin-7-yl)-3-(tert-butyl)urea |
| 293 | 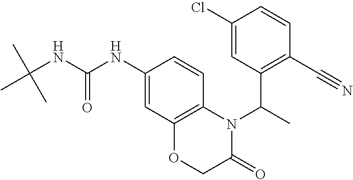 C₂₂H₂₃ClN₄O₃ | 1-(tert-butyl)-3-(4-(1-(5-chloro-2- cyanophenyl)ethyl)-3-oxo-3,4-dihydro-2H- benzo[b][1,4]oxazin-7-yl)urea |
| 294 | 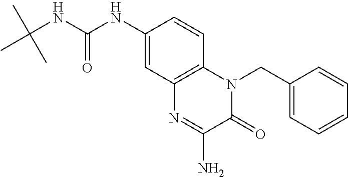 C₂₀H₂₃N₅O₂ | 1-(3-amino-1-benzyl-2-oxo-1,2- dihydroquinoxalin-6-yl)-3-(tert-butyl)urea |
| 295 | 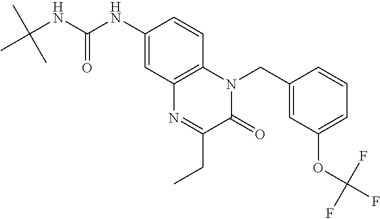 C₂₃H₂₅F₃N₄O₃ | 1-(tert-butyl)-3-(3-ethyl-2-oxo-1-(3- (trifluoromethoxy)benzyl)-1,2-dihydroquinoxalin- 6-yl)urea |
| 296 | 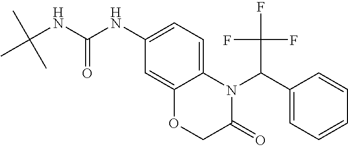 C₂₁H₂₂F₃N₃O₃ | 1-(tert-butyl)-3-(3-oxo-4-(2,2,2-trifluoro-1- phenylethyl)-3,4-dihydro-2H- benzo[b][1,4]oxazin-7-yl)urea |
| 297 | 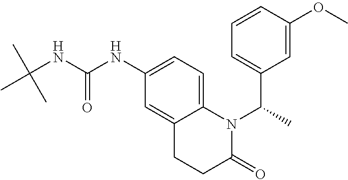 C₂₃H₂₉N₃O₃ | (S)-1-(tert-butyl)-3-(1-(1-(3- methoxyphenyl)ethyl)-2-oxo-1,2,3,4- tetrahydroquinolin-6-yl)urea |
| 298 | 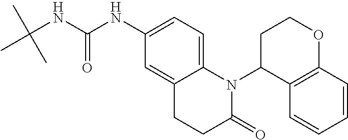 C₂₃H₂₇N₃O₃ | 1-(tert-butyl)-3-(1-(chroman-4-yl)-2-oxo-1,2,3,4- tetrahydroquinolin-6-yl)urea |
| 299 | 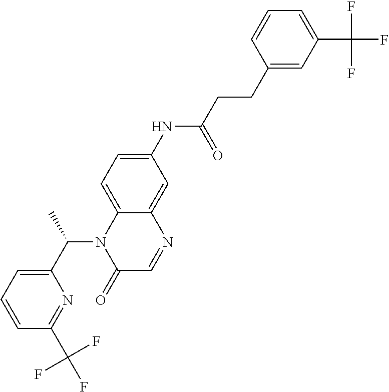 [SMILES: C[C@@H](c1cccc(C(F)(F)F)n1)n1c(=O)cnc2cc(NC(=O)CCc3cccc(C(F)(F)F)c3)ccc21] C₂₆H₂₀F₆N₄O₂ | (S)-N-(2-oxo-1-(1-(6-(trifluoromethyl)pyridin-2- yl)ethyl)-1,2-dihydroquinoxalin-6-yl)-3-(3- (trifluoromethyl)phenyl)propanamide |
| 300 | 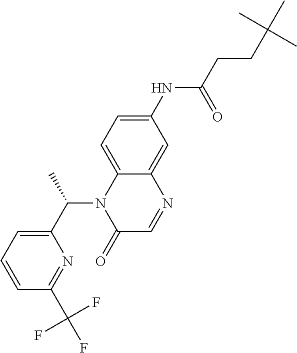 [SMILES: C[C@@H](c1cccc(C(F)(F)F)n1)n1c(=O)cnc2cc(NC(=O)CCC(C)(C)C)ccc21] C₂₃H₂₅F₃N₄O₂ | (S)-4,4-dimethyl-N-(2-oxo-1-(1-(6- (trifluoromethyl)pyridin-2-yl)ethyl)-1,2- dihydroquinoxalin-6-yl)pentanamide |
| 301 | 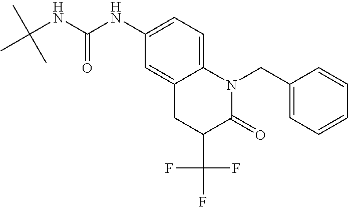 C₂₂H₂₄F₃N₃O₂ | 1-(1-benzyl-2-oxo-3-(trifluoromethyl)-1,2,3,4- tetrahydroquinolin-6-yl)-3-(tert-butyl)urea |
| 302 | 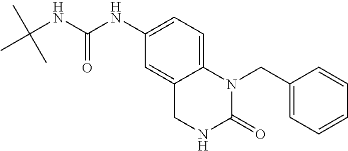 C₂₀H₂₄N₄O₂ | 1-(1-benzyl-2-oxo-1,2,3,4-tetrahydroquinazolin-6- yl)-3-(tert-butyl)urea |
| 303 | 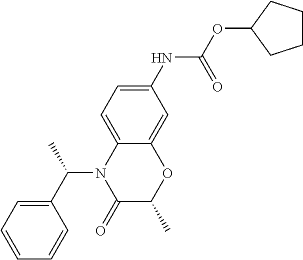 [SMILES: C[C@H]1Oc2cc(NC(=O)OC3CCCC3)ccc2N([C@@H](C)c2ccccc2)C1=O] C₂₃H₂₆N₂O₄ | cyclopentyl ((R)-2-methyl-3-oxo-4-((S)-1- phenylethyl)-3,4-dihydro-2H- benzo[b][1,4]oxazin-7-yl)carbamate |
| 304 | 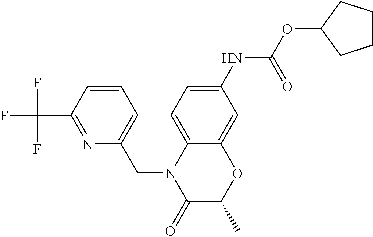 [SMILES: C[C@H]1Oc2cc(NC(=O)OC3CCCC3)ccc2N(Cc2cccc(C(F)(F)F)n2)C1=O] C₂₂H₂₂F₃N₃O₄ | cyclopentyl (R)-(2-methyl-3-oxo-4-((6- (trifluoromethyl)pyridin-2-yl)methyl)-3,4- dihydro-2H-benzo[b][1,4]oxazin-7-yl)carbamate |
| 305 | 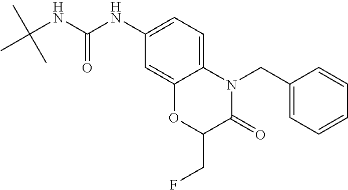 C₂₁H₂₄FN₃O₃ | 1-(4-benzyl-2-(fluoromethyl)-3-oxo-3,4-dihydro- 2H-benzo[b][1,4]oxazin-7-yl)-3-(tert-butyl)urea |
| 306 | 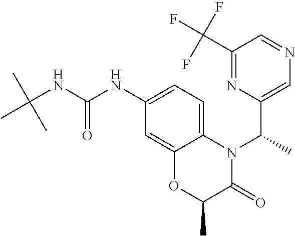 C₂₁H₂₄F₃N₅O₃ | 1-(tert-butyl)-3-((R)-2-methyl-3-oxo-4-((S)-1-(6- (trifluoromethyl)pyrazin-2-yl)ethyl)-3,4-dihydro- 2H-benzo[b][1,4]oxazin-7-yl)urea |
| 307 | 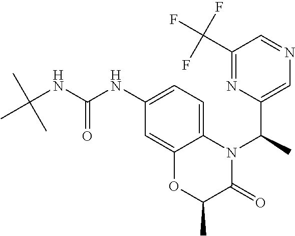 C₂₁H₂₄F₃N₅O₃ | 1-(tert-butyl)-3-((R)-2-methyl-3-oxo-4-((R)-1-(6- (trifluoromethyl)pyrazin-2-yl)ethyl)-3,4-dihydro- 2H-benzo[b][1,4]oxazin-7-yl)urea |
| 308 | 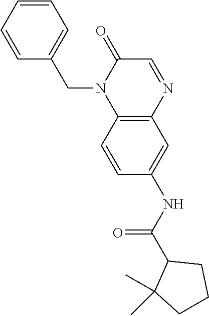 [SMILES: CC1(C)CCCC1C(=O)Nc1ccc2c(c1)ncc(=O)n2Cc1ccccc1] C₂₃H₂₅N₃O₂ | N-(1-benzyl-2-oxo-1,2-dihydroquinoxalin-6-yl)- 2,2-dimethylcyclopentane-1-carboxamide |
| 309 | 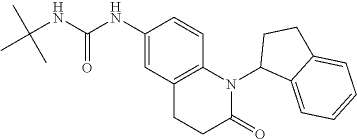 C₂₃H₂₇N₃O₂ | 1-(tert-butyl)-3-(1-(2,3-dihydro-1H-inden-1-yl)-2- oxo-1,2,3,4-tetrahydroquinolin-6-yl)urea |
| 310 | 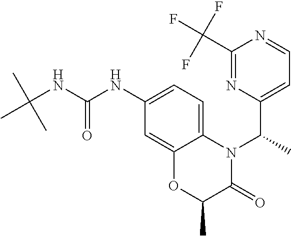 C₂₁H₂₄F₃N₅O₃ | 1-(tert-butyl)-3-((R)-2-methyl-3-oxo-4-((S)-1-(2- (trifluoromethyl)pyrimidin-4-yl)ethyl)-3,4- dihydro-2H-benzo[b][1,4]oxazin-7-yl)urea |
| 311 | 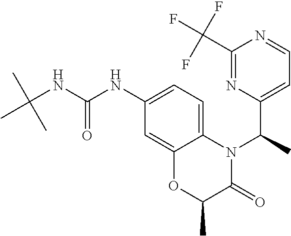 C₂₁H₂₄F₃N₅O₃ | 1-(tert-butyl)-3-((R)-2-methyl-3-oxo-4-((R)-1-(2- (trifluoromethyl)pyrimidin-4-yl)ethyl)-3,4- dihydro-2H-benzo[b][1,4]oxazin-7-yl)urea |
| 312 | 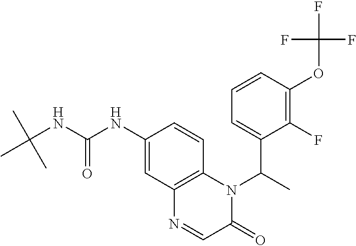 C₂₂H₂₂F₄N₄O₃ | 1-(tert-butyl)-3-(1-(1-(2-fluoro-3- (trifluoromethoxy)phenyl)ethyl)-2-oxo-1,2- dihydroquinoxalin-6-yl)urea |
| 313 | 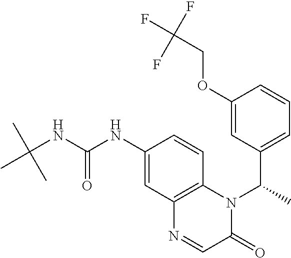 C₂₃H₂₅F₃N₄O₃ | (S)-1-(tert-butyl)-3-(2-oxo-1-(1-(3-(2,2,2- trifluoroethoxy)phenyl)ethyl)-1,2- dihydroquinoxalin-6-yl)urea |
| 314 | 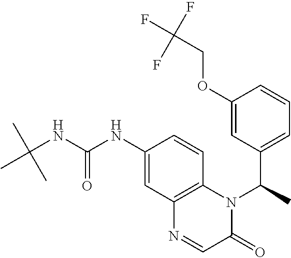 C₂₃H₂₅F₃N₄O₃ | (R)-1-(tert-butyl)-3-(2-oxo-1-(1-(3-(2,2,2- trifluoroethoxy)phenyl)ethyl)-1,2- dihydroquinoxalin-6-yl)urea |
| 315 | 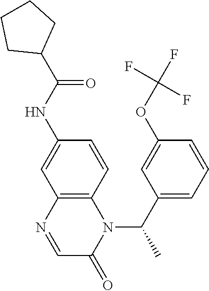 [SMILES: C[C@@H](c1cccc(OC(F)(F)F)c1)n1c(=O)cnc2cc(NC(=O)C3CCCC3)ccc21] C₂₃H₂₂F₃N₃O₃ | (S)-N-(2-oxo-1-(1-(3- (trifluoromethoxy)phenyl)ethyl)-1,2- dihydroquinoxalin-6-yl)cyclopentanecarboxamide |
| 316 | 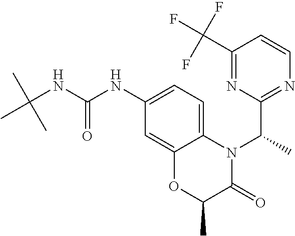 C₂₁H₂₄F₃N₅O₃ | 1-(tert-butyl)-3-((R)-2-methyl-3-oxo-4-((S)-1-(4- (trifluoromethyl)pyrimidin-2-yl)ethyl)-3,4- dihydro-2H-benzo[b][1,4]oxazin-7-yl)urea |
| 317 | 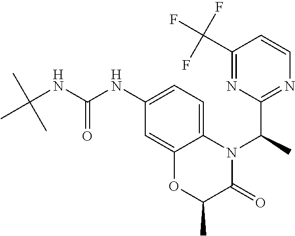 C₂₁H₂₄F₃N₅O₃ | 1-(tert-butyl)-3-((R)-2-methyl-3-oxo-4-((R)-1-(4- (trifluoromethyl)pyrimidin-2-yl)ethyl)-3,4- dihydro-2H-benzo[b][1,4]oxazin-7-yl)urea |
| 318 | 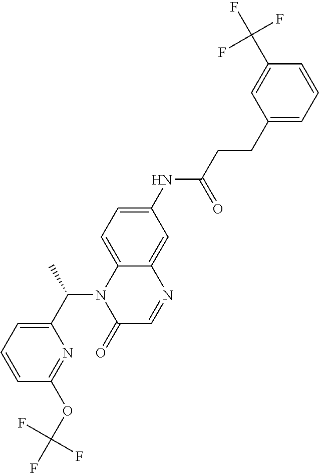 [SMILES: C[C@@H](c1cccc(OC(F)(F)F)n1)n1c(=O)cnc2cc(NC(=O)CCc3cccc(C(F)(F)F)c3)ccc21] C₂₆H₂₀F₆N₄O₃ | (S)-N-(2-oxo-1-(1-(6-(trifluoromethoxy)pyridin-2- yl)ethyl)-1,2-dihydroquinoxalin-6-yl)-3-(3- (trifluoromethyl)phenyl)propanamide |
| 319 | 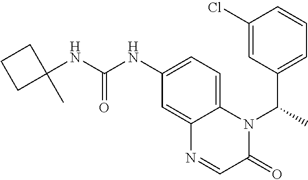 C₂₂H₂₃ClN₄O₂ | (S)-1-(1-(1-(3-chlorophenyl)ethyl)-2-oxo-1,2- dihydroquinoxalin-6-yl)-3-(1- methylcyclobutyl)urea |
| 320 | 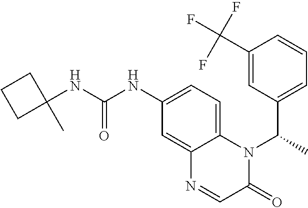 C₂₃H₂₃F₃N₄O₂ | (S)-1-(1-methylcyclobutyl)-3-(2-oxo-1-(1-(3- (trifluoromethyl)phenyl)ethyl)-1,2- dihydroquinoxalin-6-yl)urea |
| 321 | 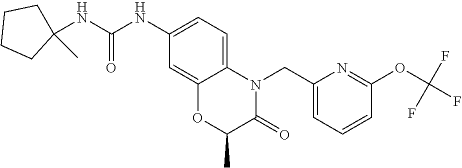 C₂₃H₂₅F₃N₄O₄ | (R)-1-(2-methyl-3-oxo-4-((6- (trifluoromethoxy)pyridin-2-yl)methyl)-3,4- dihydro-2H-benzo[b][1,4]oxazin-7-yl)-3-(1- methylcyclopentyl)urea |
| 322 | 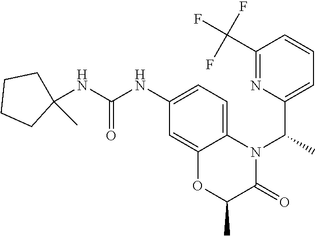 C₂₄H₂₇F₃N₄O₃ | 1-((R)-2-methyl-3-oxo-4-((S)-1-(6- (trifluoromethyl)pyridin-2-yl)ethyl)-3,4-dihydro- 2H-benzo[b][1,4]oxazin-7-yl)-3-(1- methylcyclopentyl)urea |
| 323 | 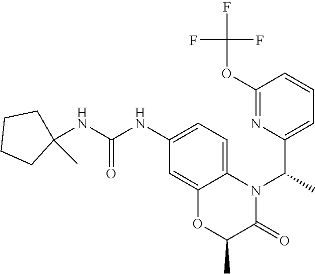 C₂₄H₂₇F₃N₄O₄ | 1-((R)-2-methyl-3-oxo-4-((S)-1-(6- (trifluoromethoxy)pyridin-2-yl)ethyl)-3,4- dihydro-2H-benzo[b][1,4]oxazin-7-yl)-3-(1- methylcyclopentyl)urea |
| 324 | 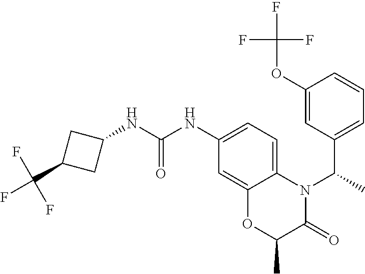 C₂₄H₂₃F₆N₃O₄ | 1-((R)-2-methyl-3-oxo-4-((S)-1-(3- (trifluoromethoxy)phenyl)ethyl)-3,4-dihydro-2H- benzo[b][1,4]oxazin-7-yl)-3-((1r,3R)-3- (trifluoromethyl)cyclobutyl)urea |
| 325 | 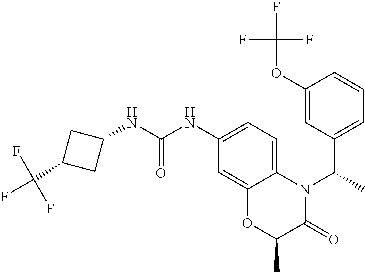 C₂₄H₂₃F₆N₃O₄ | 1-((R)-2-methyl-3-oxo-4-((S)-1-(3- (trifluoromethoxy)phenyl)ethyl)-3,4-dihydro-2H- benzo[b][1,4]oxazin-7-yl)-3-((1s,3S)-3- (trifluoromethyl)cyclobutyl)urea |
| 326 | 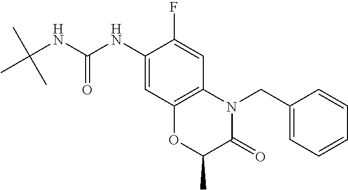 C₂₁H₂₄FN₃O₃ | (R)-1-(4-benzyl-6-fluoro-2-methyl-3-oxo-3,4- dihydro-2H-benzo[b][1,4]oxazin-7-yl)-3-(tert- butyl)urea |
| 327 | 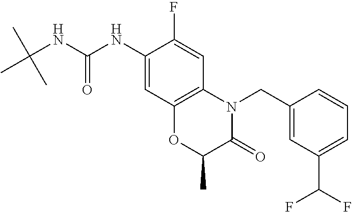 C₂₂H₂₄F₃N₃O₃ | (R)-1-(tert-butyl)-3-(4-(3- (difluoromethyl)benzyl)-6-fluoro-2-methyl-3-oxo- 3,4-dihydro-2H-benzo[b][1,4]oxazin-7-yl)urea |
| 328 | 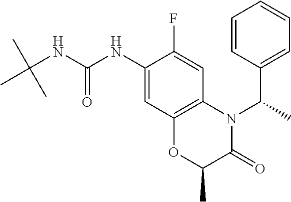 C₂₂H₂₆FN₃O₃ | 1-(tert-butyl)-3-((R)-6-fluoro-2-methyl-3-oxo-4- ((S)-1-phenylethyl)-3,4-dihydro-2H- benzo[b][1,4]oxazin-7-yl)urea |
| 329 | 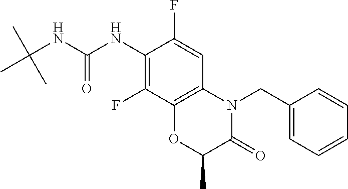 [SMILES: C[C@H]1Oc2c(cc(F)c(NC(=O)NC(C)(C)C)c2F)N(Cc2ccccc2)C1=O] C₂₁H₂₃F₂N₃O₃ | (R)-1-(4-benzyl-6,8-difluoro-2-methyl-3-oxo-3,4- dihydro-2H-benzo[b][1,4]oxazin-7-yl)-3-(tert- butyl)urea |
| 330 | 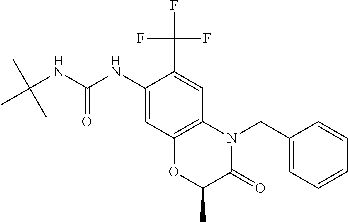 [SMILES: C[C@H]1Oc2cc(NC(=O)NC(C)(C)C)c(C(F)(F)F)cc2N(Cc2ccccc2)C1=O] C₂₂H₂₄F₃N₃O₃ | (R)-1-(4-benzyl-2-methyl-3-oxo-6- (trifluoromethyl)-3,4-dihydro-2H- benzo[b][1,4]oxazin-7-yl)-3-(tert-butyl)urea |
| 331 | 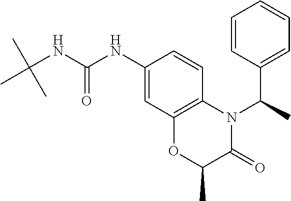 [SMILES: C[C@H](c1ccccc1)N1C(=O)[C@@H](C)Oc2cc(NC(=O)NC(C)(C)C)ccc21] C₂₂H₂₇N₃O₃ | 1-(tert-butyl)-3-((R)-2-methyl-3-oxo-4-((R)-1- phenylethyl)-3,4-dihydro-2H- benzo[b][1,4]oxazin-7-yl)urea |
| 332 | 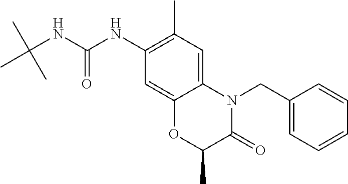 [SMILES: Cc1cc2c(cc1NC(=O)NC(C)(C)C)O[C@H](C)C(=O)N2Cc1ccccc1] C₂₂H₂₇N₃O₃ | (R)-1-(4-benzyl-2,6-dimethyl-3-oxo-3,4-dihydro- 2H-benzo[b][1,4]oxazin-7-yl)-3-(tert-butyl)urea |
| 333 | 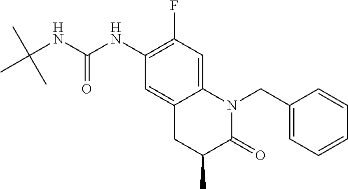 [SMILES: C[C@H]1Cc2cc(NC(=O)NC(C)(C)C)c(F)cc2N(Cc2ccccc2)C1=O] C₂₂H₂₆FN₃O₂ | (S)-1-(1-benzyl-7-fluoro-3-methyl-2-oxo-1,2,3,4- tetrahydroquinolin-6-yl)-3-(tert-butyl)urea |
| 334 | 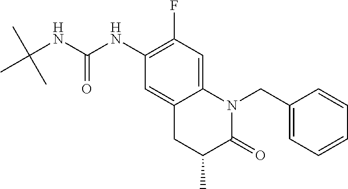 [SMILES: C[C@@H]1Cc2cc(NC(=O)NC(C)(C)C)c(F)cc2N(Cc2ccccc2)C1=O] C₂₂H₂₆FN₃O₂ | (R)-1-(1-benzyl-7-fluoro-3-methyl-2-oxo-1,2,3,4- tetrahydroquinolin-6-yl)-3-(tert-butyl)urea |
| 335 | 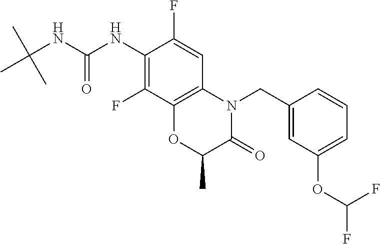 [SMILES: C[C@H]1Oc2c(cc(F)c(NC(=O)NC(C)(C)C)c2F)N(Cc2cccc(OC(F)F)c2)C1=O] C₂₂H₂₃F₄N₃O₄ | (R)-1-(tert-butyl)-3-(4-(3- (difluoromethoxy)benzyl)-6,8-difluoro-2-methyl- 3-oxo-3,4-dihydro-2H-benzo[b][1,4]oxazin-7- yl)urea |
| 336 | 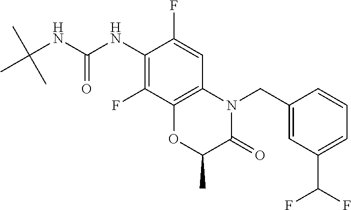 [SMILES: C[C@H]1Oc2c(cc(F)c(NC(=O)NC(C)(C)C)c2F)N(Cc2cccc(C(F)F)c2)C1=O] C₂₂H₂₃F₄N₃O₃ | (R)-1-(tert-butyl)-3-(4-(3- (difluoromethyl)benzyl)-6,8-difluoro-2-methyl-3- oxo-3,4-dihydro-2H-benzo[b][1,4]oxazin-7- yl)urea |
| 337 | 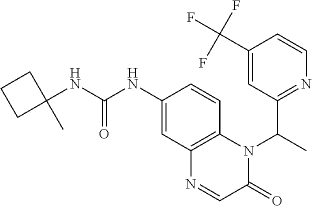 [SMILES: CC(c1cc(C(F)(F)F)ccn1)n1c(=O)cnc2cc(NC(=O)NC3(C)CCC3)ccc21] C₂₂H₂₂F₃N₅O₂ | 1-(1-methylcyclobutyl)-3-(2-oxo-1-(1-(4- (trifluoromethyl)pyridin-2-yl)ethyl)-1,2- dihydroquinoxalin-6-yl)urea |
| 338 | 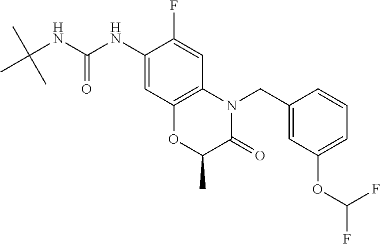 [SMILES: C[C@H]1Oc2cc(NC(=O)NC(C)(C)C)c(F)cc2N(Cc2cccc(OC(F)F)c2)C1=O] C₂₂H₂₄F₃N₃O₄ | (R)-1-(tert-butyl)-3-(4-(3- (difluoromethoxy)benzyl)-6-fluoro-2-methyl-3- oxo-3,4-dihydro-2H-benzo[b][1,4]oxazin-7- yl)urea |
| 339 | 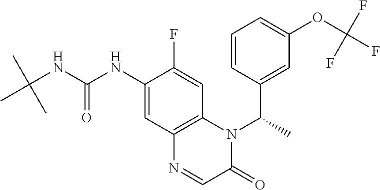 [SMILES: C[C@@H](c1cccc(OC(F)(F)F)c1)n1c(=O)cnc2cc(NC(=O)NC(C)(C)C)c(F)cc21] C₂₂H₂₂F₄N₄O₃ | (S)-1-(tert-butyl)-3-(7-fluoro-2-oxo-1-(1-(3- (trifluoromethoxy)phenyl)ethyl)-1,2- dihydroquinoxalin-6-yl)urea |
| 340 | 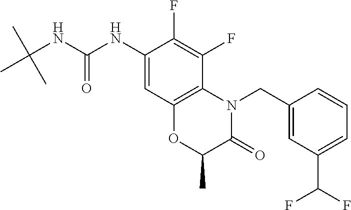 [SMILES: C[C@H]1Oc2cc(NC(=O)NC(C)(C)C)c(F)c(F)c2N(Cc2cccc(C(F)F)c2)C1=O] C₂₂H₂₃F₄N₃O₃ | (R)-1-(tert-butyl)-3-(4-(3- (difluoromethyl)benzyl)-5,6-difluoro-2-methyl-3- oxo-3,4-dihydro-2H-benzo[b][1,4]oxazin-7- yl)urea |
| 341 | 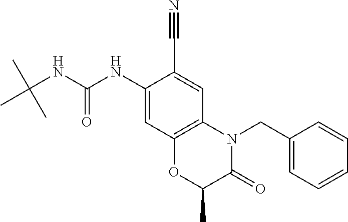 [SMILES: C[C@H]1Oc2cc(NC(=O)NC(C)(C)C)c(C#N)cc2N(Cc2ccccc2)C1=O] C₂₂H₂₄N₄O₃ | (R)-1-(4-benzyl-6-cyano-2-methyl-3-oxo-3,4- dihydro-2H-benzo[b][1,4]oxazin-7-yl)-3-(tert- butyl)urea |
| 342 | 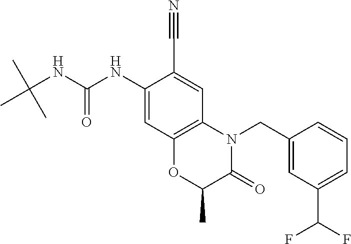 [SMILES: C[C@H]1Oc2cc(NC(=O)NC(C)(C)C)c(C#N)cc2N(Cc2cccc(C(F)F)c2)C1=O] C₂₃H₂₄F₂N₄O₃ | (R)-1-(tert-butyl)-3-(6-cyano-4-(3- (difluoromethyl)benzyl)-2-methyl-3-oxo-3,4- dihydro-2H-benzo[b][1,4]oxazin-7-yl)urea |
| 343 | 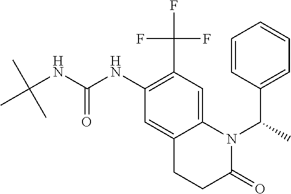 [SMILES: C[C@@H](c1ccccc1)N1C(=O)CCc2cc(NC(=O)NC(C)(C)C)c(C(F)(F)F)cc21] C₂₃H₂₆F₃N₃O₂ | (S)-1-(tert-butyl)-3-(2-oxo-1-(1-phenylethyl)-7- (trifluoromethyl)-1,2,3,4-tetrahydroquinolin-6- yl)urea |
| 344 | 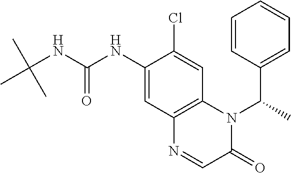 [SMILES: C[C@@H](c1ccccc1)n1c(=O)cnc2cc(NC(=O)NC(C)(C)C)c(Cl)cc21] C₂₁H₂₃ClN₄O₂ | (S)-1-(tert-butyl)-3-(7-chloro-2-oxo-1-(1- phenylethyl)-1,2-dihydroquinoxalin-6-yl)urea |
| 345 | 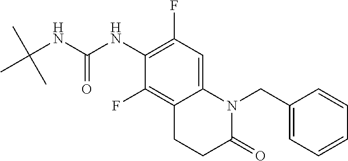 [SMILES: CC(C)(C)NC(=O)Nc1c(F)cc2c(c1F)CCC(=O)N2Cc1ccccc1] C₂₁H₂₃F₂N₃O₂ | 1-(1-benzyl-5,7-difluoro-2-oxo-1,2,3,4- tetrahydroquinolin-6-yl)-3-(tert-butyl)urea |
| 346 | 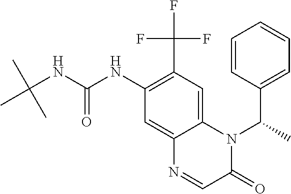 [SMILES: C[C@@H](c1ccccc1)n1c(=O)cnc2cc(NC(=O)NC(C)(C)C)c(C(F)(F)F)cc21] C₂₂H₂₃F₃N₄O₂ | (S)-1-(tert-butyl)-3-(2-oxo-1-(1-phenylethyl)-7- (trifluoromethyl)-1,2-dihydroquinoxalin-6-yl)urea |
| 347 | 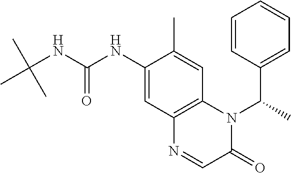 [SMILES: Cc1cc2c(cc1NC(=O)NC(C)(C)C)ncc(=O)n2[C@@H](C)c1ccccc1] C₂₂H₂₆N₄O₂ | (S)-1-(tert-butyl)-3-(7-methyl-2-oxo-1-(1- phenylethyl)-1,2-dihydroquinoxalin-6-yl)urea |
| 348 | 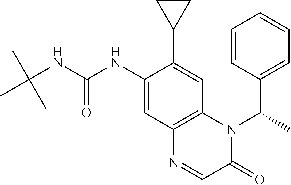 [SMILES: C[C@@H](c1ccccc1)n1c(=O)cnc2cc(NC(=O)NC(C)(C)C)c(C3CC3)cc21] C₂₄H₂₈N₄O₂ | (S)-1-(tert-butyl)-3-(7-cyclopropyl-2-oxo-1-(1- phenylethyl)-1,2-dihydroquinoxalin-6-yl)urea |
| 349 | 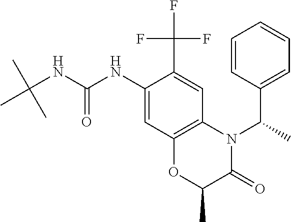 [SMILES: C[C@H]1Oc2cc(NC(=O)NC(C)(C)C)c(C(F)(F)F)cc2N([C@@H](C)c2ccccc2)C1=O] C₂₃H₂₆F₃N₃O₃ | 1-(tert-butyl)-3-((R)-2-methyl-3-oxo-4-((S)-1- phenylethyl)-6-(trifluoromethyl)-3,4-dihydro-2H- benzo[b][1,4]oxazin-7-yl)urea |
| 350 | 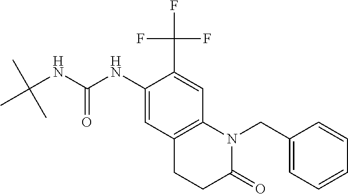 [SMILES: CC(C)(C)NC(=O)Nc1cc2c(cc1C(F)(F)F)N(Cc1ccccc1)C(=O)CC2] C₂₂H₂₄F₃N₃O₂ | 1-(1-benzyl-2-oxo-7-(trifluoromethyl)-1,2,3,4- tetrahydroquinolin-6-yl)-3-(tert-butyl)urea |
| 351 | 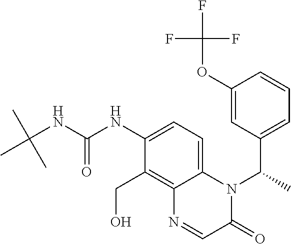 [SMILES: C[C@@H](c1cccc(OC(F)(F)F)c1)n1c(=O)cnc2c(CO)c(NC(=O)NC(C)(C)C)ccc21] C₂₃H₂₅F₃N₄O₄ | (S)-1-(tert-butyl)-3-(5-(hydroxymethyl)-2-oxo-1- (1-(3-(trifluoromethoxy)phenyl)ethyl)-1,2- dihydroquinoxalin-6-yl)urea |
| 352 | 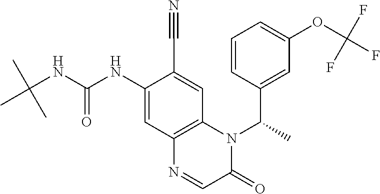 [SMILES: C[C@@H](c1cccc(OC(F)(F)F)c1)n1c(=O)cnc2cc(NC(=O)NC(C)(C)C)c(C#N)cc21] C₂₃H₂₂F₃N₅O₃ | (S)-1-(tert-butyl)-3-(7-cyano-2-oxo-1-(1-(3- (trifluoromethoxy)phenyl)ethyl)-1,2- dihydroquinoxalin-6-yl)urea |
| 353 | 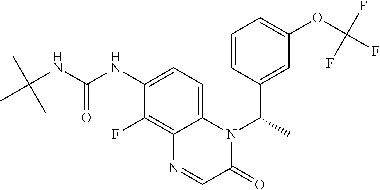 [SMILES: C[C@@H](c1cccc(OC(F)(F)F)c1)n1c(=O)cnc2c(F)c(NC(=O)NC(C)(C)C)ccc21] C₂₂H₂₂F₄N₄O₃ | (S)-1-(tert-butyl)-3-(5-fluoro-2-oxo-1-(1-(3- (trifluoromethoxy)phenyl)ethyl)-1,2- dihydroquinoxalin-6-yl)urea |
| 354 | 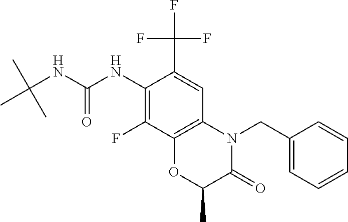 [SMILES: C[C@H]1Oc2c(cc(C(F)(F)F)c(NC(=O)NC(C)(C)C)c2F)N(Cc2ccccc2)C1=O] C₂₂H₂₃F₄N₃O₃ | (R)-1-(4-benzyl-8-fluoro-2-methyl-3-oxo-6- (trifluoromethyl)-3,4-dihydro-2H- benzo[b][1,4]oxazin-7-yl)-3-(tert-butyl)urea |
| 355 | 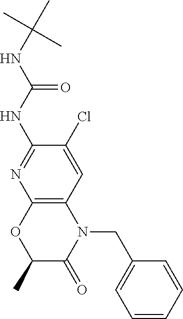 [SMILES: C[C@H]1Oc2nc(NC(=O)NC(C)(C)C)c(Cl)cc2N(Cc2ccccc2)C1=O] C₂₀H₂₃ClN₄O₃ | (R)-1-(1-benzyl-7-chloro-3-methyl-2-oxo-2,3- dihydro-1H-pyrido[2,3-b][1,4]oxazin-6-yl)-3- (tert-butyl)urea |
| 356 | 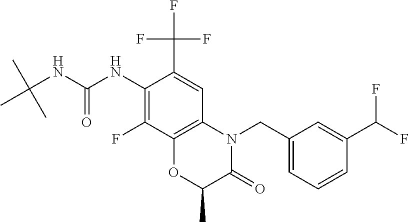 [SMILES: C[C@H]1Oc2c(cc(C(F)(F)F)c(NC(=O)NC(C)(C)C)c2F)N(Cc2cccc(C(F)F)c2)C1=O] C₂₃H₂₃F₆N₃O₃ | (R)-1-(tert-butyl)-3-(4-(3- (difluoromethyl)benzyl)-8-fluoro-2-methyl-3-oxo- 6-(trifluoromethyl)-3,4-dihydro-2H- benzo[b][1,4]oxazin-7-yl)urea |
| 357 | 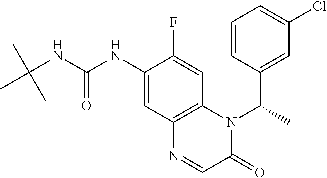 [SMILES: C[C@@H](c1cccc(Cl)c1)n1c(=O)cnc2cc(NC(=O)NC(C)(C)C)c(F)cc21] C₂₁H₂₂ClFN₄O₂ | (S)-1-(tert-butyl)-3-(1-(1-(3-chlorophenyl)ethyl)- 7-fluoro-2-oxo-1,2-dihydroquinoxalin-6-yl)urea |
| 358 | 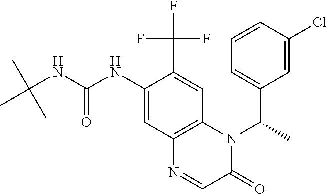 [SMILES: C[C@@H](c1cccc(Cl)c1)n1c(=O)cnc2cc(NC(=O)NC(C)(C)C)c(C(F)(F)F)cc21] C₂₂H₂₂ClF₃N₄O₂ | (S)-1-(tert-butyl)-3-(1-(1-(3-chlorophenyl)ethyl)- 2-oxo-7-(trifluoromethyl)-1,2-dihydroquinoxalin- 6-yl)urea |
| 359 | 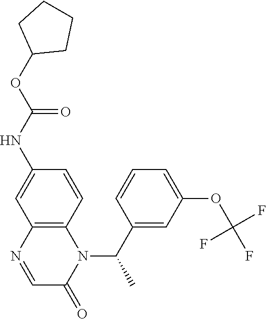 [SMILES: C[C@@H](c1cccc(OC(F)(F)F)c1)n1c(=O)cnc2cc(NC(=O)OC3CCCC3)ccc21] C₂₃H₂₂F₃N₃O₄ | cyclopentyl (S)-(2-oxo-1-(1-(3- (trifluoromethoxy)phenyl)ethyl)-1,2- dihydroquinoxalin-6-yl)carbamate |
| 360 | 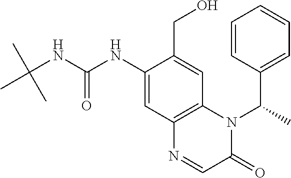 [SMILES: C[C@@H](c1ccccc1)n1c(=O)cnc2cc(NC(=O)NC(C)(C)C)c(CO)cc21] C₂₂H₂₆N₄O₃ | (S)-1-(tert-butyl)-3-(7-(hydroxymethyl)-2-oxo-1- (1-phenylethyl)-1,2-dihydroquinoxalin-6-yl)urea |
| 361 | 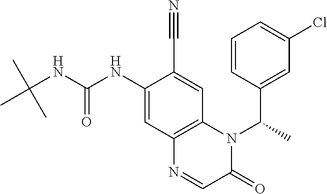 [SMILES: C[C@@H](c1cccc(Cl)c1)n1c(=O)cnc2cc(NC(=O)NC(C)(C)C)c(C#N)cc21] C₂₂H₂₂ClN₅O₂ | (S)-1-(tert-butyl)-3-(1-(1-(3-chlorophenyl)ethyl)- 7-cyano-2-oxo-1,2-dihydroquinoxalin-6-yl)urea |
| 362 | 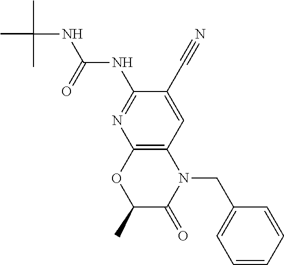 [SMILES: C[C@H]1Oc2nc(NC(=O)NC(C)(C)C)c(C#N)cc2N(Cc2ccccc2)C1=O] C₂₁H₂₃N₅O₃ | (R)-1-(1-benzyl-7-cyano-3-methyl-2-oxo-2,3- dihydro-1H-pyrido[2,3-b][1,4]oxazin-6-yl)-3- (tert-butyl)urea |
| 363 | 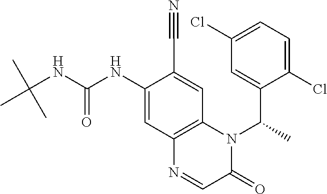 [SMILES: C[C@@H](c1cc(Cl)ccc1Cl)n1c(=O)cnc2cc(NC(=O)NC(C)(C)C)c(C#N)cc21] C₂₂H₂₁Cl₂N₅O₂ | (S)-1-(tert-butyl)-3-(7-cyano-1-(1-(2,5- dichlorophenyl)ethyl)-2-oxo-1,2- dihydroquinoxalin-6-yl)urea |
| 364 | 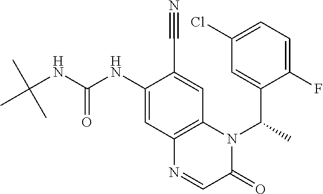 [SMILES: C[C@@H](c1cc(Cl)ccc1F)n1c(=O)cnc2cc(NC(=O)NC(C)(C)C)c(C#N)cc21] C₂₂H₂₁ClFN₅O₂ | (S)-1-(tert-butyl)-3-(1-(1-(5-chloro-2- fluorophenyl)ethyl)-7-cyano-2-oxo-1,2- dihydroquinoxalin-6-yl)urea |
| 365 | 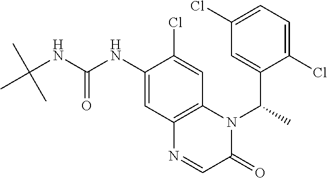 [SMILES: C[C@@H](c1cc(Cl)ccc1Cl)n1c(=O)cnc2cc(NC(=O)NC(C)(C)C)c(Cl)cc21] C₂₁H₂₁Cl₃N₄O₂ | (S)-1-(tert-butyl)-3-(7-chloro-1-(1-(2,5- dichlorophenyl)ethyl)-2-oxo-1,2- dihydroquinoxalin-6-yl)urea |
| 366 | 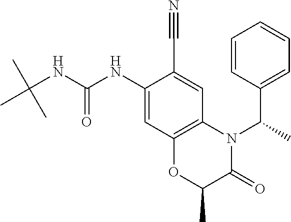 [SMILES: C[C@H]1Oc2cc(NC(=O)NC(C)(C)C)c(C#N)cc2N([C@@H](C)c2ccccc2)C1=O] C₂₃H₂₆N₄O₃ | 1-(tert-butyl)-3-((R)-6-cyano-2-methyl-3-oxo-4- ((S)-1-phenylethyl)-3,4-dihydro-2H- benzo[b][1,4]oxazin-7-yl)urea |
| 367 | 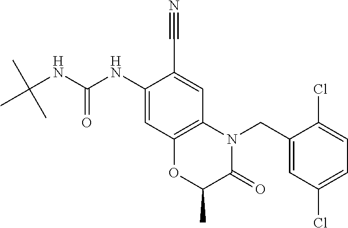 [SMILES: C[C@H]1Oc2cc(NC(=O)NC(C)(C)C)c(C#N)cc2N(Cc2cc(Cl)ccc2Cl)C1=O] C₂₂H₂₂Cl₂N₄O₃ | (R)-1-(tert-butyl)-3-(6-cyano-4-(2,5- dichlorobenzyl)-2-methyl-3-oxo-3,4-dihydro-2H- benzo[b][1,4]oxazin-7-yl)urea |
| 368 | 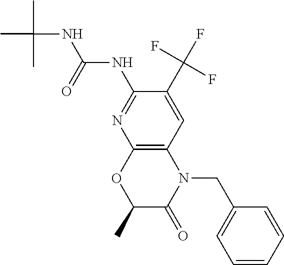 [SMILES: C[C@H]1Oc2nc(NC(=O)NC(C)(C)C)c(C(F)(F)F)cc2N(Cc2ccccc2)C1=O] C₂₁H₂₃F₃N₄O₃ | (R)-1-(1-benzyl-3-methyl-2-oxo-7- (trifluoromethyl)-2,3-dihydro-1H-pyrido[2,3- b][1,4]oxazin-6-yl)-3-(tert-butyl)urea |
| 369 | 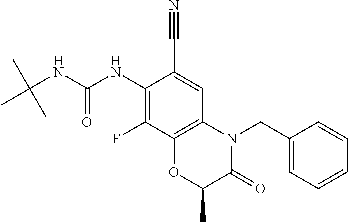 [SMILES: C[C@H]1Oc2c(cc(C#N)c(NC(=O)NC(C)(C)C)c2F)N(Cc2ccccc2)C1=O] C₂₂H₂₃FN₄O₃ | (R)-1-(4-benzyl-6-cyano-8-fluoro-2-methyl-3- oxo-3,4-dihydro-2H-benzo[b][1,4]oxazin-7-yl)-3- (tert-butyl)urea |
| 370 | 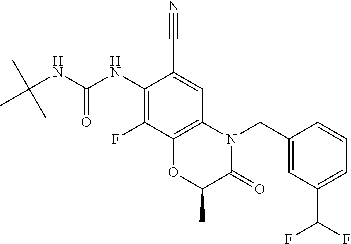 [SMILES: C[C@H]1Oc2c(cc(C#N)c(NC(=O)NC(C)(C)C)c2F)N(Cc2cccc(C(F)F)c2)C1=O] C₂₃H₂₃F₃N₄O₃ | (R)-1-(tert-butyl)-3-(6-cyano-4-(3- (difluoromethyl)benzyl)-8-fluoro-2-methyl-3-oxo- 3,4-dihydro-2H-benzo[b][1,4]oxazin-7-yl)urea |
| 371 | 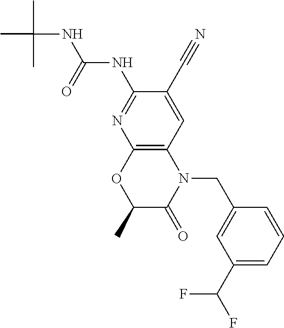 [SMILES: C[C@H]1Oc2nc(NC(=O)NC(C)(C)C)c(C#N)cc2N(Cc2cccc(C(F)F)c2)C1=O] C₂₂H₂₃F₂N₅O₃ | (R)-1-(tert-butyl)-3-(7-cyano-1-(3- (difluoromethyl)benzyl)-3-methyl-2-oxo-2,3- dihydro-1H-pyrido[2,3-b][1,4]oxazin-6-yl)urea |
| 372 | 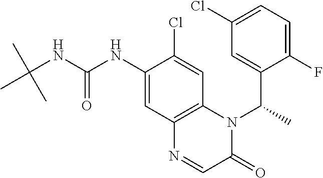 [SMILES: C[C@@H](c1cc(Cl)ccc1F)n1c(=O)cnc2cc(NC(=O)NC(C)(C)C)c(Cl)cc21] C₂₁H₂₁Cl₂FN₄O₂ | (S)-1-(tert-butyl)-3-(7-chloro-1-(1-(5-chloro-2- fluorophenyl)ethyl)-2-oxo-1,2-dihydroquinoxalin- 6-yl)urea |
| 373 | 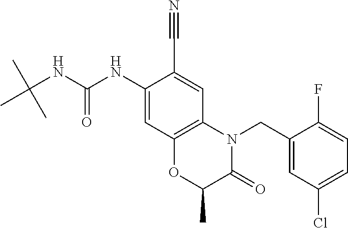 [SMILES: C[C@H]1Oc2cc(NC(=O)NC(C)(C)C)c(C#N)cc2N(Cc2cc(Cl)ccc2F)C1=O] C₂₂H₂₂ClFN₄O₃ | (R)-1-(tert-butyl)-3-(4-(5-chloro-2-fluorobenzyl)- 6-cyano-2-methyl-3-oxo-3,4-dihydro-2H- benzo[b][1,4]oxazin-7-yl)urea |
| 374 | 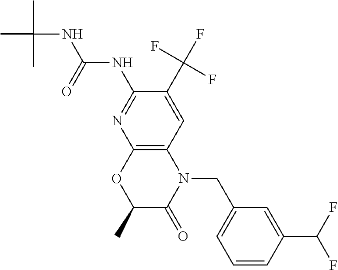 [SMILES: C[C@H]1Oc2nc(NC(=O)NC(C)(C)C)c(C(F)(F)F)cc2N(Cc2cccc(C(F)F)c2)C1=O] C₂₂H₂₃F₅N₄O₃ | (R)-1-(tert-butyl)-3-(1-(3- (difluoromethyl)benzyl)-3-methyl-2-oxo-7- (trifluoromethyl)-2,3-dihydro-1H-pyrido[2,3- b][1,4]oxazin-6-yl)urea |
| 375 | 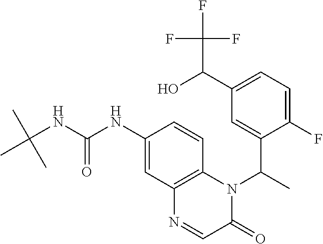 [SMILES: CC(c1cc(C(O)C(F)(F)F)ccc1F)n1c(=O)cnc2cc(NC(=O)NC(C)(C)C)ccc21] C₂₃H₂₄F₄N₄O₃ | 1-(tert-butyl)-3-(1-(1-(2-fluoro-5-(2,2,2-trifluoro- 1-hydroxyethyl)phenyl)ethyl)-2-oxo-1,2- dihydroquinoxalin-6-yl)urea |
| 376 | 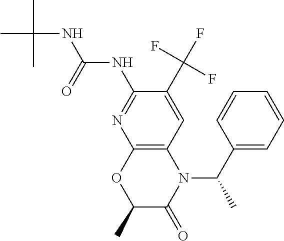 [SMILES: C[C@H]1Oc2nc(NC(=O)NC(C)(C)C)c(C(F)(F)F)cc2N([C@@H](C)c2ccccc2)C1=O] C₂₂H₂₅F₃N₄O₃ | 1-(tert-butyl)-3-((R)-3-methyl-2-oxo-1-((S)-1- phenylethyl)-7-(trifluoromethyl)-2,3-dihydro-1H- pyrido[2,3-b][1,4]oxazin-6-yl)urea |
| 377 | 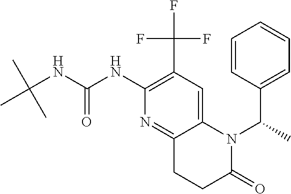 [SMILES: C[C@@H](c1ccccc1)N1C(=O)CCc2nc(NC(=O)NC(C)(C)C)c(C(F)(F)F)cc21] C₂₂H₂₅F₃N₄O₂ | (S)-1-(tert-butyl)-3-(6-oxo-5-(1-phenylethyl)-3- (trifluoromethyl)-5,6,7,8-tetrahydro-1,5- naphthyridin-2-yl)urea |
| 378 | 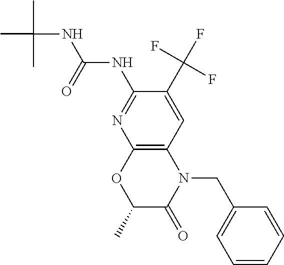 [SMILES: C[C@@H]1Oc2nc(NC(=O)NC(C)(C)C)c(C(F)(F)F)cc2N(Cc2ccccc2)C1=O] C₂₁H₂₃F₃N₄O₃ | (R)-1-(1-benzyl-3-methyl-2-oxo-7- (trifluoromethyl)-2,3-dihydro-1H-pyrido[2,3- b][1,4]oxazin-6-yl)-3-(tert-butyl)urea |

or a pharmaceutically acceptable salt thereof.

75. A compound selected from the following table:

| | | |
|---|---|---|
| 1 | 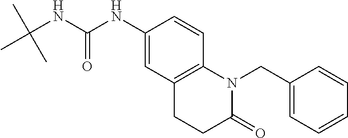 [SMILES: CC(C)(C)NC(=O)Nc1ccc2c(c1)CCC(=O)N2Cc1ccccc1] | 1-(1-benzyl-2-oxo-1,2,3,4-tetrahydroquinolin-6-yl)-3-(tert- butyl)urea |
| 2 | 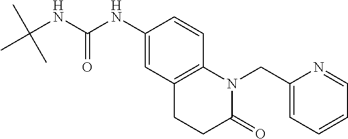 [SMILES: CC(C)(C)NC(=O)Nc1ccc2c(c1)CCC(=O)N2Cc1ccccn1] | 1-(tert-buty1)-3-(2-oxo-1-(pyridin-2-ylmethyl)-1,2,3,4- tetrahydroquinolin-6-yl)urea |
| 3 | 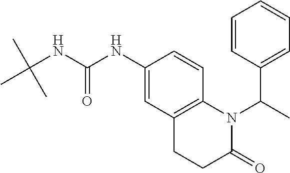 [SMILES: CC(c1ccccc1)N1C(=O)CCc2cc(NC(=O)NC(C)(C)C)ccc21] | 1-(tert-butyl)-3-(2-oxo-1-(1-phenylethyl)-1,2,3,4- tetrahydroquinolin-6-yl)urea |
| 4 | 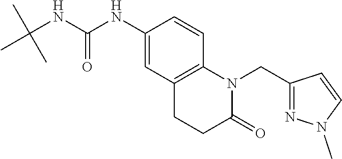 [SMILES: Cn1ccc(CN2C(=O)CCc3cc(NC(=O)NC(C)(C)C)ccc32)n1] | 1-(tert-butyl)-3-(1-((1-methyl-1H-pyrazol-3-yl)methyl)-2-oxo- 1,2,3,4-tetrahydroquinolin-6-yl)urea |
| 5 | 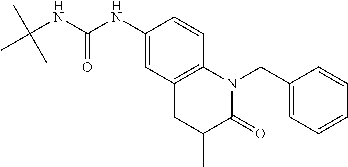 [SMILES: CC1Cc2cc(NC(=O)NC(C)(C)C)ccc2N(Cc2ccccc2)C1=O] | 1-(1-benzyl-3-methyl-2-oxo-1,2,3,4-tetrahydroquinolin-6-yl)-3- (tert-butyl)urea |
| 6 | 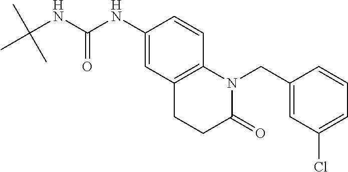 [SMILES: CC(C)(C)NC(=O)Nc1ccc2c(c1)CCC(=O)N2Cc1cccc(Cl)c1] | 1-(tert-butyl)-3-(1-(3-chlorobenzyl)-2-oxo-1,2,3,4- tetrahydroquinolin-6-yl)urea |
| 7 | 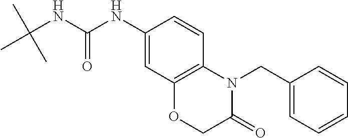 [SMILES: CC(C)(C)NC(=O)Nc1ccc2c(c1)OCC(=O)N2Cc1ccccc1] | 1-(4-benzyl-3-oxo-3,4-dihydro-2H-benzo[b][1,4]oxazin-7-yl)-3- (tert-butyl)urea |
| 8 | 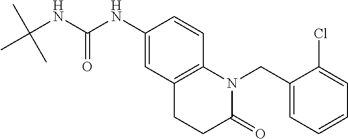 [SMILES: CC(C)(C)NC(=O)Nc1ccc2c(c1)CCC(=O)N2Cc1ccccc1Cl] | 1-(tert-butyl)-3-(1-(2-chlorobenzyl)-2-oxo-1,2,3,4- tetrahydroquinolin-6-yl)urea |
| 9 | 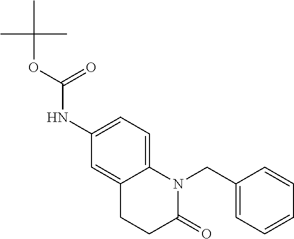 [SMILES: CC(C)(C)OC(=O)Nc1ccc2c(c1)CCC(=O)N2Cc1ccccc1] | tert-butyl (1-benzyl-2-oxo-1,2,3,4-tetrahydroquinolin-6- yl)carbamate |
| 10 | 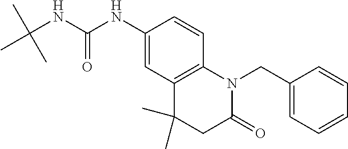 [SMILES: CC(C)(C)NC(=O)Nc1ccc2c(c1)C(C)(C)CC(=O)N2Cc1ccccc1] | 1-(1-benzyl-4,4-dimethyl-2-oxo-1,2,3,4-tetrahydroquinolin-6-yl)- 3-(tert-butyl)urea |
| 11 | 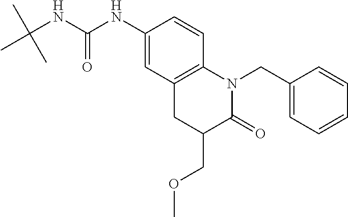 [SMILES: COCC1Cc2cc(NC(=O)NC(C)(C)C)ccc2N(Cc2ccccc2)C1=O] | 1-(1-benzyl-3-(methoxymethyl)-2-oxo-1,2,3,4- tetrahydroquinolin-6-y1)-3-(tert-butyl)urea |
| 12 | 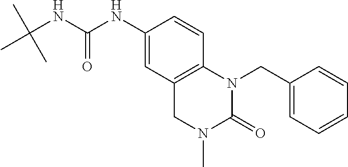 [SMILES: CN1Cc2cc(NC(=O)NC(C)(C)C)ccc2N(Cc2ccccc2)C1=O] | 1-(1-benzyl-3-methyl-2-oxo-1,2,3,4-tetrahydroquinazolin-6-yl)- 3-(tert-butyl)urea |
| 13 | 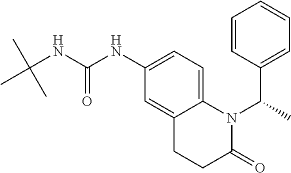 [SMILES: C[C@@H](c1ccccc1)N1C(=O)CCc2cc(NC(=O)NC(C)(C)C)ccc21] | (S)-1-(tert-butyl)-3-(2-oxo-1-(1-phenylethyl)-1,2,3,4- tetrahydroquinolin-6-yl)urea |
| 14 | 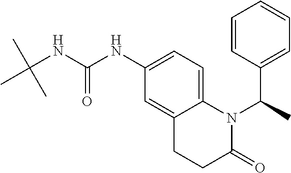 [SMILES: C[C@H](c1ccccc1)N1C(=O)CCc2cc(NC(=O)NC(C)(C)C)ccc21] | (R)-1-(tert-butyl)-3-(2-oxo-1-(1-phenylethyl)-1,2,3,4- tetrahydroquinolin-6-yl)urea |
| 15 | 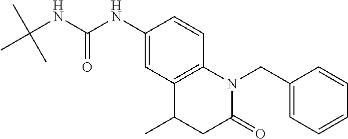 [SMILES: CC1CC(=O)N(Cc2ccccc2)c2ccc(NC(=O)NC(C)(C)C)cc21] | 1-(1-benzyl-4-methyl-2-oxo-1,2,3,4-tetrahydroquinolin-6-yl)-3- (tert-butyl)urea |
| 16 | 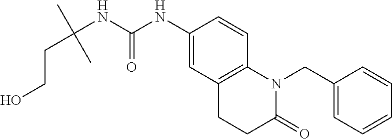 [SMILES: CC(C)(CCO)NC(=O)Nc1ccc2c(c1)CCC(=O)N2Cc1ccccc1] | 1-(1-benzyl-2-oxo-1,2,3,4-tetrahydroquinolin-6-yl)-3-(4- hydroxy-2-methylbutan-2-yl)urea |
| 17 | 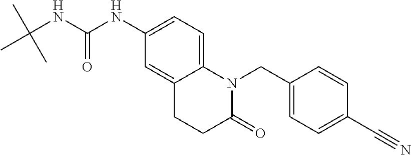 [SMILES: CC(C)(C)NC(=O)Nc1ccc2c(c1)CCC(=O)N2Cc1ccc(C#N)cc1] | 1-(tert-butyl)-3-(1-(4-cyanobenzyl)-2-oxo-1,2,3,4- tetrahydroquinolin-6-yl)urea |
| 18 | 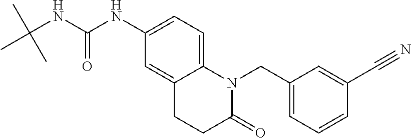 [SMILES: CC(C)(C)NC(=O)Nc1ccc2c(c1)CCC(=O)N2Cc1cccc(C#N)c1] | 1-(tert-butyl)-3-(1-(3-cyanobenzyl)-2-oxo-1,2,3,4- tetrahydroquinolin-6-yl)urea |
| 19 | 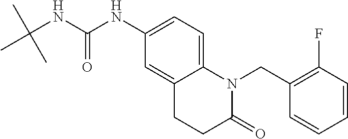 [SMILES: CC(C)(C)NC(=O)Nc1ccc2c(c1)CCC(=O)N2Cc1ccccc1F] | 1-(tert-butyl)-3-(1-(2-fluorobenzyl)-2-oxo-1,2,3,4- tetrahydroquinolin-6-yl)urea |
| 20 | 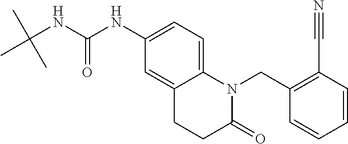 [SMILES: CC(C)(C)NC(=O)Nc1ccc2c(c1)CCC(=O)N2Cc1ccccc1C#N] | 1-(tert-butyl)-3-(1-(2-cyanobenzyl)-2-oxo-1,2,3,4- tetrahydroquinolin-6-yl)urea |
| 21 | 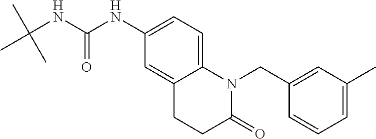 [SMILES: Cc1cccc(CN2C(=O)CCc3cc(NC(=O)NC(C)(C)C)ccc32)c1] | 1-(tert-butyl)-3-(1-(3-methylbenzyl)-2-oxo-1,2,3,4- tetrahydroquinolin-6-yl)urea |
| 22 | 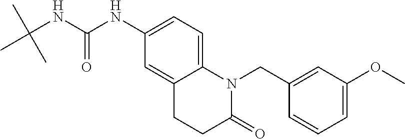 [SMILES: COc1cccc(CN2C(=O)CCc3cc(NC(=O)NC(C)(C)C)ccc32)c1] | 1 -(tert-butyl)-3-(1-(3-methoxybenzyl)-2-oxo-1,2,3,4- tetrahydroquinolin-6-yl)urea |
| 23 | 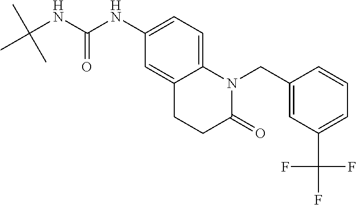 [SMILES: CC(C)(C)NC(=O)Nc1ccc2c(c1)CCC(=O)N2Cc1cccc(C(F)(F)F)c1] | 1-(tert-butyl)-3-(2-oxo-1-(3-(trifluoromethyl)benzyl)-1,2,3,4- tetrahydroquinolin-6-yl)urea |
| 24 | 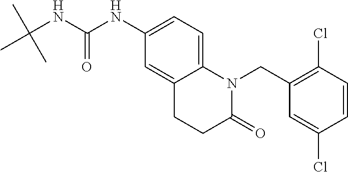 [SMILES: CC(C)(C)NC(=O)Nc1ccc2c(c1)CCC(=O)N2Cc1cc(Cl)ccc1Cl] | 1-(tert-butyl)-3-(1-(2,5-dichlorobenzyl)-2-oxo-1,2,3,4- tetrahydroquinolin-6-yl)urea |
| 25 | 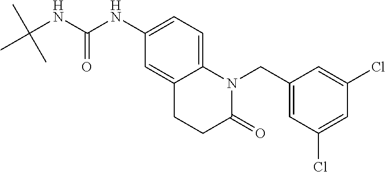 [SMILES: CC(C)(C)NC(=O)Nc1ccc2c(c1)CCC(=O)N2Cc1cc(Cl)cc(Cl)c1] | 1-(tert-butyl)-3-(1-(3,5-dichlorobenzyl)-2-oxo-1,2,3,4- tetrahydroquinolin-6-yl)urea |
| 26 | 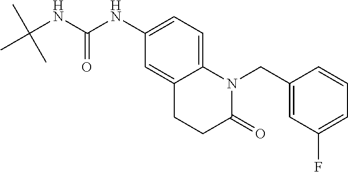 [SMILES: CC(C)(C)NC(=O)Nc1ccc2c(c1)CCC(=O)N2Cc1cccc(F)c1] | 1-(tert-butyl)-3-(1-(3-fluorobenzyl)-2-oxo-1,2,3,4- tetrahydroquinolin-6-yl)urea |
| 27 | 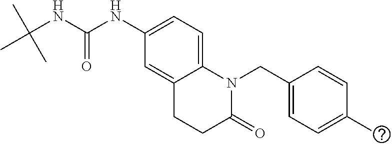 [SMILES: Cc1ccc(CN2C(=O)CCc3cc(NC(=O)NC(C)(C)C)ccc32)cc1] | 1-(tert-buty1)-3-(1-(4-fluorobenzyl)-2-oxo-1,2,3,4- tetrahydroquinolin-6-yl)urea |
| 28 | 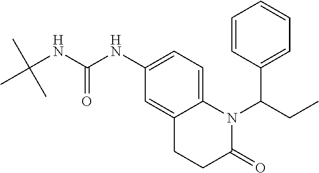 [SMILES: CCC(c1ccccc1)N1C(=O)CCc2cc(NC(=O)NC(C)(C)C)ccc21] | 1-(tert-butyl)-3-(2-oxo-1-(1-phenylpropyl)-1,2,3,4- tetrahydroquinolin-6-yl)urea |
| 29 | 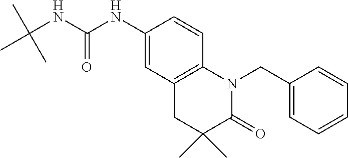 [SMILES: CC(C)(C)NC(=O)Nc1ccc2c(c1)CC(C)(C)C(=O)N2Cc1ccccc1] | 1-(1-benzyl-3,3-dimethyl-2-oxo-1,2,3,4-tetrahydroquinolin-6-yl)- 3-(tert-butyl)urea |
| 30 | 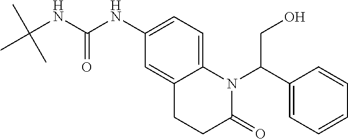 [SMILES: CC(C)(C)NC(=O)Nc1ccc2c(c1)CCC(=O)N2C(CO)c1ccccc1] | 1-(tert-butyl)-3-(1-(2-hydroxy-1-phenylethyl)-2-oxo-1,2,3,4- tetrahydroquinolin-6-yl)urea |
| 31 | 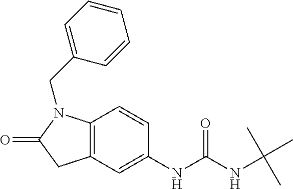 [SMILES: CC(C)(C)NC(=O)Nc1ccc2c(c1)CC(=O)N2Cc1ccccc1] | 1-(1-benzyl-2-oxoindolin-5-yl)-3-(tert-butyl)urea |
| 32 | 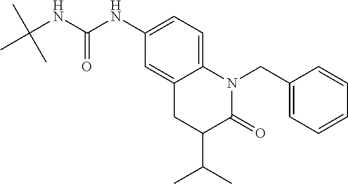 [SMILES: CC(C)C1Cc2cc(NC(=O)NC(C)(C)C)ccc2N(Cc2ccccc2)C1=O] | 1-(1-benzyl-3-isopropyl-2-oxo-1,2,3,4-tetrahydroquinolin-6-yl)- 3-(tert-butyl)urea |
| 33 | 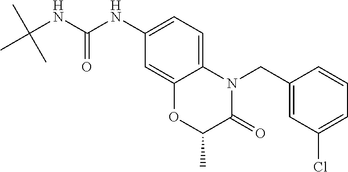 [SMILES: C[C@@H]1Oc2cc(NC(=O)NC(C)(C)C)ccc2N(Cc2cccc(Cl)c2)C1=O] | 1-(tert-butyl)-3-(4-(3-chlorobenzyl)-3-oxo-3,4-dihydro-2H- benzo[b][1,4]oxazin-7-yl)urea |
| 34 | 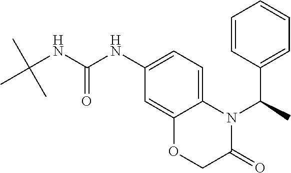 [SMILES: C[C@H](c1ccccc1)N1C(=O)COc2cc(NC(=O)NC(C)(C)C)ccc21] | (R)-1-(tert-butyl)-3-(3-oxo-4-(1-phenylethyl)-3,4-dihydro-2H- benzo[b][1,4]oxazin-7-yl)urea |
| 35 | 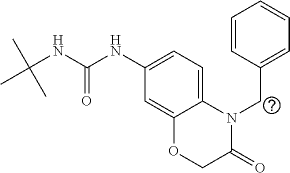 [SMILES: CC(C)(C)NC(=O)Nc1ccc2c(c1)OCC(=O)N2Cc1ccccc1] | (S)-1-(tert-butyl)-3-(3-oxo-4-(1-phenylethyl)-3,4-dihydro-2H- benzo[b][1,4]oxazin-7-yl)urea |
| | C₂₁H₂₅N₃O₃ | |
| 36 | 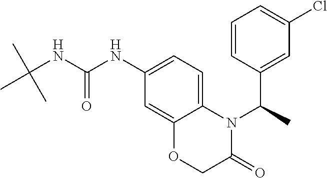 [SMILES: C[C@H](c1cccc(Cl)c1)N1C(=O)COc2cc(NC(=O)NC(C)(C)C)ccc21] | (R)-1-(tert-butyl)-3-(4-(1-(3-chlorophenyl)ethyl)-3-oxo-3,4- dihydro-2H-benzo[b][1,4]oxazin-7-yl)urea |
| 37 | 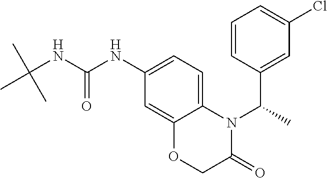 [SMILES: C[C@@H](c1cccc(Cl)c1)N1C(=O)COc2cc(NC(=O)NC(C)(C)C)ccc21] | (S)-1-(tert-butyl)-3-(4-(1-(3-chlorophenyl)ethyl)-3-oxo-3,4- dihydro-2H-benzo[b][1,4]oxazin-7-yl)urea |
| 38 | 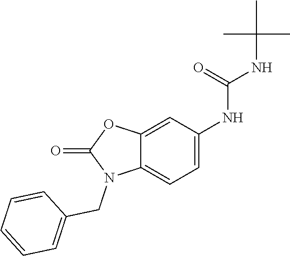 [SMILES: CC(C)(C)NC(=O)Nc1ccc2c(c1)oc(=O)n2Cc1ccccc1] | 1-(3-benzyl-2-oxo-2,3-dihydrobenzo[d]oxazol-6-yl)-3-(tert- butyl)urea |
| 39 | 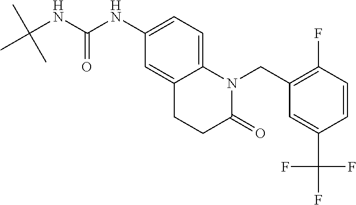 [SMILES: CC(C)(C)NC(=O)Nc1ccc2c(c1)CCC(=O)N2Cc1cc(C(F)(F)F)ccc1F] | 1-(tert-butyl)-3-(1-(2-fluoro-5-(trifluoromethyl)benzyl)-2-oxo- 1,2,3,4-tetrahydroquinolin-6-yl)urea |
| 40 | 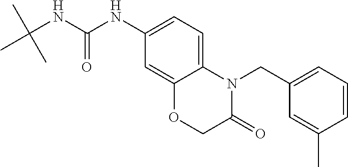 [SMILES: Cc1cccc(CN2C(=O)COc3cc(NC(=O)NC(C)(C)C)ccc32)c1] | 1-(tert-butyl)-3-(4-(3-methylbenzyl)-3-oxo-3,4-dihydro-2H- benzo[b][1,4]oxazin-7-yl)urea |
| 41 | 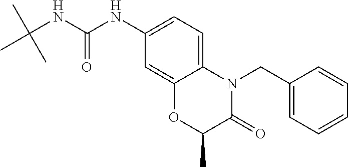 [SMILES: C[C@H]1Oc2cc(NC(=O)NC(C)(C)C)ccc2N(Cc2ccccc2)C1=O] | (R)-1-(4-benzyl-2-methyl-3-oxo-3,4-dihydro-2H- benzo[b][1,4]oxazin-7-yl)-3-(tert-butyl)urea |
| 42 | 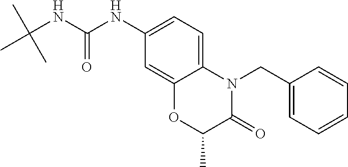 [SMILES: C[C@@H]1Oc2cc(NC(=O)NC(C)(C)C)ccc2N(Cc2ccccc2)C1=O] | (S)-1-(4-benzyl-2-methyl-3-oxo-3,4-dihydro-2H- benzo[b][1,4]oxazin-7-yl)-3-(tert-butyl)urea |
| 43 | 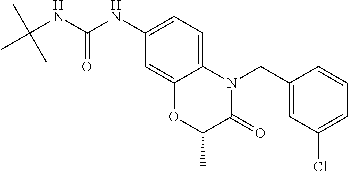 [SMILES: C[C@@H]1Oc2cc(NC(=O)NC(C)(C)C)ccc2N(Cc2cccc(Cl)c2)C1=O] | (S)-1-(tert-butyl)-3-(4-(3-chlorobenzyl)-2-methyl-3-oxo-3,4- dihydro-2H-benzo[b][1,4]oxazin-7-yl)urea |
| 44 | 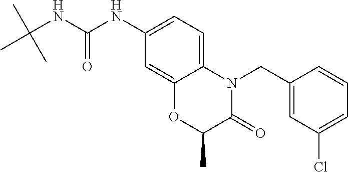 [SMILES: C[C@H]1Oc2cc(NC(=O)NC(C)(C)C)ccc2N(Cc2cccc(Cl)c2)C1=O] | (R)-1-(tert-butyl)-3-(4-(3-chlorobenzyl)-2-methyl-3-oxo-3,4- dihydro-2H-benzo[b][1,4]oxazin-7-yl)urea |
| 45 | 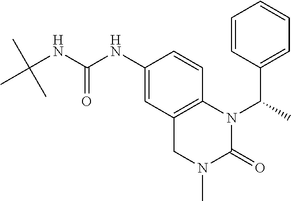 [SMILES: C[C@@H](c1ccccc1)N1C(=O)N(C)Cc2cc(NC(=O)NC(C)(C)C)ccc21] | (S)-1-(tert-butyl)-3-(3-methyl-2-oxo-1-(1-phenylethyl)-1,2,3,4- tetrahydroquinazolin-6-yl)urea |
| 46 | 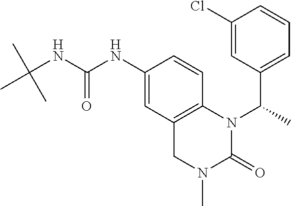 [SMILES: C[C@@H](c1cccc(Cl)c1)N1C(=O)N(C)Cc2cc(NC(=O)NC(C)(C)C)ccc21] | (S)-1-(tert-butyl)-3-(1-(1-(3-chlorophenyl)ethyl)-3-methyl-2-oxo- 1,2,3,4-tetrahydroquinazolin-6-yl)urea |
| 47 | 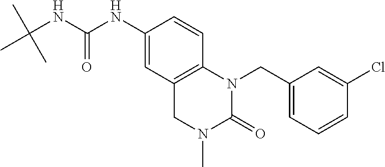 [SMILES: CN1Cc2cc(NC(=O)NC(C)(C)C)ccc2N(Cc2cccc(Cl)c2)C1=O] | 1-(tert-butyl)-3-(1-(3-chlorobenzyl)-3-methyl-2-oxo-1,2,3,4- tetrahydroquinazolin-6-yl)urea |
| 48 | 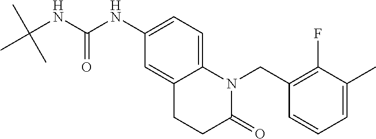 [SMILES: Cc1cccc(CN2C(=O)CCc3cc(NC(=O)NC(C)(C)C)ccc32)c1F] | 1-(tert-butyl)-3-(1-(2-fluoro-3-methylbenzyl)-2-oxo-1,2,3,4- tetrahydroquinolin-6-yl)urea |
| 49 | 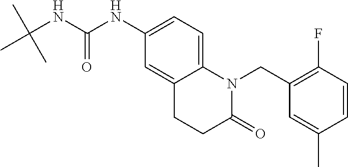 [SMILES: Cc1ccc(F)c(CN2C(=O)CCc3cc(NC(=O)NC(C)(C)C)ccc32)c1] | 1-(tert-butyl)-3-(1-(2-fluoro-5-methylbenzyl)-2-oxo-1,2,3,4- tetrahydroquinolin-6-yl)urea |
| 50 | 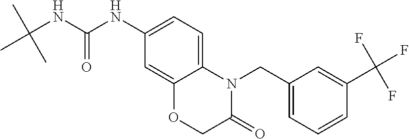 [SMILES: CC(C)(C)NC(=O)Nc1ccc2c(c1)OCC(=O)N2Cc1cccc(C(F)(F)F)c1] | 1 -(tert-butyl)-3-(3-oxo-4-(3-(trifluoromethyl)benzyl)-3,4- dihydro-2H-benzo[b][1,4]oxazin-7-yl)urea |
| 51 | 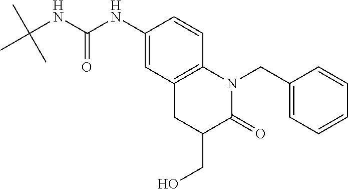 [SMILES: CC(C)(C)NC(=O)Nc1ccc2c(c1)CC(CO)C(=O)N2Cc1ccccc1] | 1-(1-benzyl-3-(hydroxymethyl)-2-oxo-1,2,3,4- tetrahydroquinolin-6-y1)-3-(tert-butyl)urea |
| 52 | 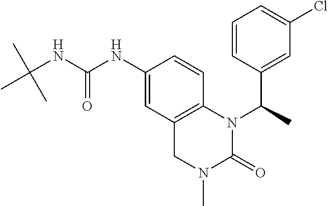 [SMILES: C[C@H](c1cccc(Cl)c1)N1C(=O)N(C)Cc2cc(NC(=O)NC(C)(C)C)ccc21] | (R)-1-(tert-butyl)-3-(1-(1-(3-chlorophenyl)ethyl)-3-methyl-2-oxo- 1,2,3,4-tetrahydroquinazolin-6-yl)urea |
| 53 | 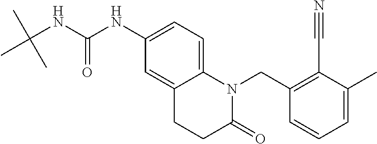 [SMILES: Cc1cccc(CN2C(=O)CCc3cc(NC(=O)NC(C)(C)C)ccc32)c1C#N] | 1 -(tert-butyl)-3-(1-(2-cyano-3-methylbenzyl)-2-oxo-1,2,3,4- tetrahydroquinolin-6-yl)urea |
| 54 | 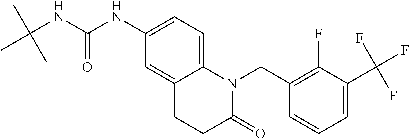 [SMILES: CC(C)(C)NC(=O)Nc1ccc2c(c1)CCC(=O)N2Cc1cccc(C(F)(F)F)c1F] | 1-(tert-butyl)-3-(1-(2-fluoro-3-(trifluoromethyl)benzyl)-2-oxo- 1,2,3,4-tetrahydroquinolin-6-yl)urea |
| 55 | 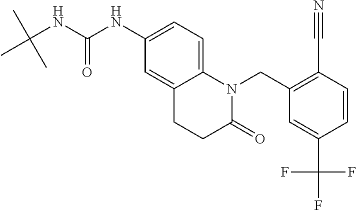 [SMILES: CC(C)(C)NC(=O)Nc1ccc2c(c1)CCC(=O)N2Cc1cc(C(F)(F)F)ccc1C#N] | 1-(tert-butyl)-3-(1-(2-cyano-5-(trifluoromethyl)benzyl)-2-oxo- 1,2,3,4-tetrahydroquinolin-6-yl)urea |
| 56 | 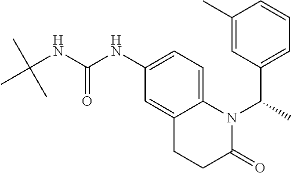 [SMILES: Cc1cccc([C@H](C)N2C(=O)CCc3cc(NC(=O)NC(C)(C)C)ccc32)c1] | (S)-1-(tert-butyl)-3-(2-oxo-1-(1-(m-tolyl)ethyl)-1,2,3,4- tetrahydroquinolin-6-yl)urea |
| 57 | 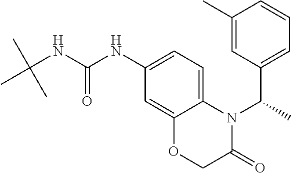 [SMILES: Cc1cccc([C@H](C)N2C(=O)COc3cc(NC(=O)NC(C)(C)C)ccc32)c1] | (S)-1-(tert-butyl)-3-(3-oxo-4-(1-(m-tolyl)ethyl)-3,4-dihydro-2H- benzo[b][1,4]oxazin-7-yl)urea |
| 58 | 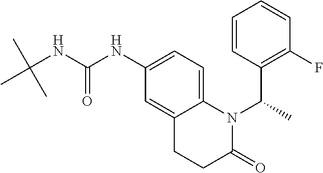 [SMILES: C[C@@H](c1ccccc1F)N1C(=O)CCc2cc(NC(=O)NC(C)(C)C)ccc21] | (S)-1-(tert-butyl)-3-(1-(1-(2-fluorophenyl)ethyl)-2-oxo-1,2,3,4- tetrahydroquinolin-6-yl)urea |
| 59 | 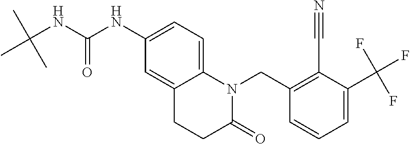 [SMILES: CC(C)(C)NC(=O)Nc1ccc2c(c1)CCC(=O)N2Cc1cccc(C(F)(F)F)c1C#N] | 1-(tert-butyl)-3-(1-(2-cyano-3-(trifluoromethyl)benzyl)-2-oxo- 1,2,3,4-tetrahydroquinolin-6-yl)urea |
| 60 | 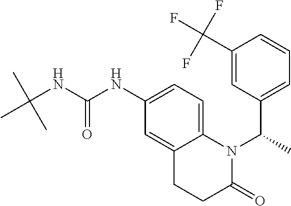 [SMILES: C[C@@H](c1cccc(C(F)(F)F)c1)N1C(=O)CCc2cc(NC(=O)NC(C)(C)C)ccc21] | (S)-1-(tert-butyl)-3-(2-oxo-1-(1-(3- (trifluoromethyl)phenyl)ethyl)-1,2,3,4-tetrahydroquinolin-6- yl)urea |
| 61 | 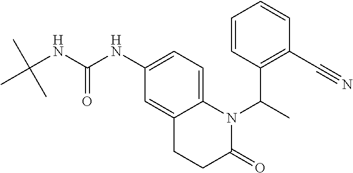 [SMILES: CC(c1ccccc1C#N)N1C(=O)CCc2cc(NC(=O)NC(C)(C)C)ccc21] | (S)-1-(tert-butyl)-3-(1-(1-(2-cyanophenyl)ethyl)-2-oxo-1,2,3,4- tetrahydroquinolin-6-yl)urea |
| 62 | 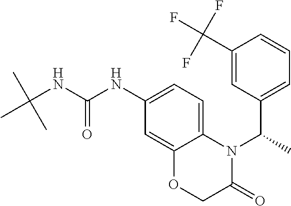 [SMILES: C[C@@H](c1cccc(C(F)(F)F)c1)N1C(=O)COc2cc(NC(=O)NC(C)(C)C)ccc21] | (S)-1-(tert-butyl)-3-(3-oxo-4-(1-(3- (trifluoromethyl)phenyl)ethyl)-3,4-dihydro-2H- benzo[b][1,4]oxazin-7-yl)urea |
| 63 | 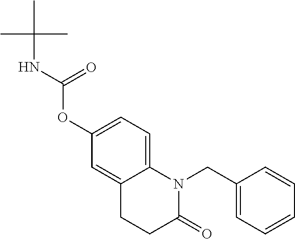 [SMILES: CC(C)(C)NC(=O)Oc1ccc2c(c1)CCC(=O)N2Cc1ccccc1] | 1-benzyl-2-oxo-1,2,3,4-tetrahydroquinolin-6-yl tert- butylcarbamate |
| 64 | 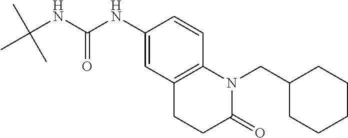 [SMILES: CC(C)(C)NC(=O)Nc1ccc2c(c1)CCC(=O)N2CC1CCCCC1] | 1-(tert-butyl)-3-(1-(cyclohexylmethyl)-2-oxo-1,2,3,4- tetrahydroquinolin-6-yl)urea |
| 65 | 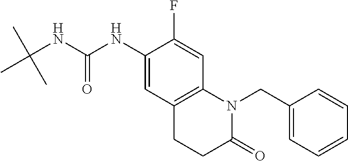 [SMILES: CC(C)(C)NC(=O)Nc1cc2c(cc1F)N(Cc1ccccc1)C(=O)CC2] | 1-(1-benzyl-7-fluoro-2-oxo-1,2,3,4-tetrahydroquinolin-6-yl)-3- (tert-butyl)urea |
| 66 | 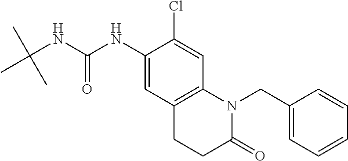 [SMILES: CC(C)(C)NC(=O)Nc1cc2c(cc1Cl)N(Cc1ccccc1)C(=O)CC2] | 1-(1-benzyl-7-chloro-2-oxo-1,2,3,4-tetrahydroquinolin-6-yl)-3- (tert-butyl)urea |
| 67 | 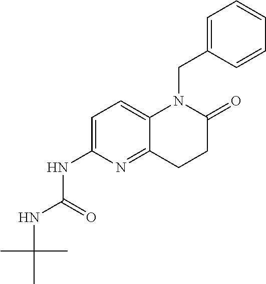 [SMILES: CC(C)(C)NC(=O)Nc1ccc2c(n1)CCC(=O)N2Cc1ccccc1] | 1-(5-benzyl-6-oxo-5,6,7,8-tetrahydro-1,5-naphthyridin-2-yl)-3- (tert-butyl)urea |
| 68 | 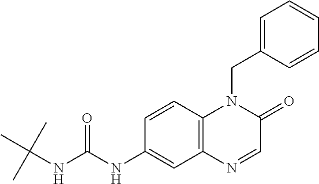 [SMILES: CC(C)(C)NC(=O)Nc1ccc2c(c1)ncc(=O)n2Cc1ccccc1] | 1-(1-benzyl-2-oxo-1,2-dihydroquinoxalin-6-yl)-3-(tert-butyl)urea |
| 69 | 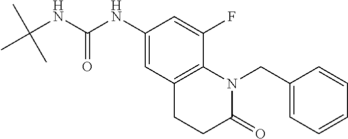 [SMILES: CC(C)(C)NC(=O)Nc1cc(F)c2c(c1)CCC(=O)N2Cc1ccccc1] | 1-(1-benzyl-8-fluoro-2-oxo-1,2,3,4-tetrahydroquinolin-6-yl)-3- (tert-butyl)urea; and |
| 70 | 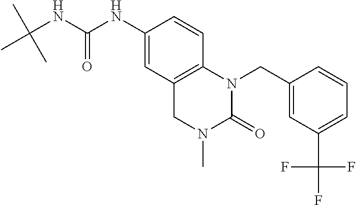 [SMILES: CN1Cc2cc(NC(=O)NC(C)(C)C)ccc2N(Cc2cccc(C(F)(F)F)c2)C1=O] | 1-(tert-butyl)-3-(3-methyl-2-oxo-1-(3-(trifluoromethyl)benzyl)- 1,2,3,4-tetrahydroquinazolin-6-yl)urea |

or a pharmaceutically acceptable salt thereof.

76. A pharmaceutical composition, comprising a compound of any one of claims 1 to 75; and at least one pharmaceutically acceptable excipient.

77. A method of inhibiting a thyroid stimulating hormone receptor, comprising administering to a subject in need thereof a therapeutically effective amount of a compound of any one of claims 1 to 75.

78. A method of treating hyperthyroidism, comprising administering to a subject in need thereof a therapeutically effective amount of a compound of any one of claims 1 to 75.

79. The method of claim 78, wherein the subject has Graves' disease.

80. The method of claim 79 wherein the subject has Graves' ophthalmopathy.

81. The method of claim 79 or 80, wherein the subject has Graves' dermopathy.

82. The method of claim 78, wherein the subject has thyroid cancer.
